(19) United States
(12) Patent Application Publication
(10) Pub. No.: US 2025/0197934 A1
(43) Pub. Date: Jun. 19, 2025

(54) RET GENE FUSIONS AND USES THEREOF

(71) Applicant: Foundation Medicine, Inc., Boston, MA (US)

(72) Inventors: Erica GORNSTEIN, Melrose, MA (US); Rachel ERBACH, Arlington, MA (US); Xin LIU, Boston, MA (US); Richard Sheng Poe HUANG, Cary, NC (US); Zheng KUANG, Boston, MA (US); Vamsi PARIMI, Cary, NC (US)

(73) Assignee: Foundation Medicine, Inc., Boston, MA (US)

(21) Appl. No.: 18/988,176

(22) Filed: Dec. 19, 2024

Related U.S. Application Data
(63) Continuation of application No. PCT/US2023/069544, filed on Jun. 30, 2023.
(60) Provisional application No. 63/415,931, filed on Oct. 13, 2022.
(60) Provisional application No. 63/358,042, filed on Jul. 1, 2022.

(51) Int. Cl. C12Q 1/6874; C12N 15/10; C12Q 1/686; C12Q 1/6886; G16H 15/00; G16H 20/10

(52) U.S. Cl. CPC C12Q 1/6874; C12N 15/1093; C12Q 1/686; C12Q 1/6886; G16H 15/00; G16H 20/10; C12Q 2600/106; C12Q 2600/156

(57) ABSTRACT

Provided herein are rearranged during transfection (RET) fusion nucleic acid molecules and RET fusion polypeptides, methods related to detecting RET fusion nucleic acid molecules and RET fusion polypeptides in cancer, as well as methods of treatment and uses related thereto. Detection of a RET fusion nucleic acid molecule or a RET fusion polypeptide can be used to identify individuals that may benefit from treatment with an anti-cancer therapy.

### CROSS REFERENCE TO RELATED APPLICATIONS

This patent application is a continuation application of International Application No. PCT/US2023/069544, filed internationally on Jun. 30, 2023, which claims the priority benefit of U.S. Provisional Application No. 63/358,042, filed on Jul. 1, 2022, and 63/415,931, filed on Oct. 13, 2022, the disclosures of which are herein incorporated by reference in their entirety.

### REFERENCE TO AN ELECTRONIC SEQUENCE LISTING

The content of the electronic sequence listing (197102008901seqlist.xml; Size: 434,603 bytes; and Date of Creation: Dec. 16, 2024) is herein incorporated by reference in its entirety.

### TECHNICAL FIELD

Provided herein are rearranged during transfection (RET) fusion nucleic acid molecules and polypeptides, methods related to detecting such RET fusion nucleic acid molecules and polypeptides, as well as methods of diagnosis/treatment and uses related thereto.

### BACKGROUND

Kinases activated by gene fusions are established oncogenic drivers and therapeutic targets, and have been associated with both hematopoietic malignancies and solid tumors. Kinase gene fusions have also been observed in patients following initial treatment with targeted therapies, suggesting that kinase fusions may be an acquired resistance mechanism, and that patients with such fusions could benefit from strategies that target the acquired kinase fusion. See, e.g. Xu et al., Cancer Manag Res (2019) 11:6343-51; Piotrowska et al., Cancer Discov (2018) 8 (12): 1529-39; Schrock et al., J Thorac Oncol (2018) 13 (9): 1312-23; and Schrock et al., J Thorac Oncol 2019; 14 (2): 255-64).

RET (Rearranged during transfection) encodes a receptor tyrosine kinase primarily expressed in cells of the nervous system. It has been identified as a proto-oncogene that results in transformation of cells upon recombination with a partner gene (Takahashi et al. (1985) Cell, 42 (2), 581-588). In addition, RET fusions involving an N-terminal partner gene that can promote dimerization, and the kinase domain of RET (exons 12-18; Shaw et al. (2013) Nature reviews. Cancer, 13 (11), 772-787) have been characterized as activating and oncogenic (Ju et al. (2012) Genome research, 22 (3), 436-445; Belli et al. (2020) Clinical cancer research, 26 (23), 6102-6111; Powell et al. (1998) Cancer research, 58 (23), 5523-5528; Jhiang et al. (2000) Oncogene, 19 (49), 5590-5597; Matsubara et al. (2012) Journal of thoracic oncology 7 (12), 1872-1876; Takeuchi et al. (2012) Nature medicine, 18 (3), 378-381; Fusco et al. (1987) Nature, 328 (6126), 170-172; Lipson et al. (2012) Nature medicine, 18 (3), 382-384; Kohno et al. (2012) Nature medicine, 18 (3), 375-377; Chang et al. (2017) Yonsei medical journal, 58 (1), 9-18; and Das et al. (2017) Cell reports, 20 (10), 2368-2383). Certain other RET rearrangements have also been reported to retain capacity to dimerize through self-association of the RET transmembrane domain and have been shown to be mildly transforming (Takahashi et al. (1988) Oncogene, 3 (5), 571-578; and Kjaer et al. (2006) Oncogene, 25 (53), 7086-7095).

RET fusions and rearrangements may predict responses to certain therapies. For example, certain RET fusions have been shown to be clinically sensitive to RET targeted therapies (see, e.g. Drilon et al., PL02.08 Registrational Results of LIBRETTO-001: A Phase 1/2 Trial of LOXO-292 in Patients with RET Fusion-Positive Lung Cancers, Journal of Thoracic Oncology, Volume 14, Issue 10, S6-S7; Gainor et al., Registrational dataset from the phase I/II ARROW trial of pralsetinib (BLU-667) in patients (pts) with advanced RET fusion+non-small cell lung cancer (NSCLC), Journal of Clinical Oncology 2020 38: 15_suppl, 9515-9515; Wirth et al. (2020) The New England journal of medicine, 383 (9), 825-835; Gautschi et al. (2017) Journal of clinical oncology 35 (13), 1403-1410; Belli et al. (2020) Clinical cancer research 26 (23), 6102-6111; Drilon et al. (2013) Cancer discovery, 3 (6), 630-635; Wang et al. (2016) Clinical cancer research 22 (24), 6061-6068; and Li et al. (2017) Clinical cancer research, 23 (12), 2981-2990).

Thus, there is a need in the art for characterizing the cancer landscape of RET fusions, and for developing methods, compositions, and assays for evaluating and treating patients with such fusions.

All references cited herein, including patents, patent applications and publications, are hereby incorporated by reference in their entirety. To the extent that any reference incorporated by reference conflicts with the instant disclosure, the instant disclosure shall control.

### SUMMARY OF THE INVENTION

In one aspect, provided herein is a method of identifying an individual having a cancer who may benefit from a treatment comprising a rearranged during transfection (RET)-targeted therapy, the method comprising detecting in a sample from the individual a RET fusion nucleic acid molecule, or a RET fusion polypeptide encoded by the RET fusion nucleic acid molecule, wherein: (a) the RET fusion nucleic acid molecule comprises a fusion between a RET gene, or a portion thereof, and a gene listed in Table 1, or a portion thereof, or (b) the RET fusion nucleic acid molecule comprises a fusion between a RET gene, or a portion thereof, and a gene listed in Table 2, or a portion thereof, and the cancer is the cancer corresponding to the gene listed in Table 2, and wherein detection of the RET fusion nucleic acid molecule, or the RET fusion polypeptide encoded by the RET fusion nucleic acid molecule, in the sample identifies the individual as one who may benefit from the treatment comprising the RET-targeted therapy.

In another aspect, provided herein is a method of selecting a therapy for an individual having a cancer, the method comprising detecting in a sample from the individual a RET fusion nucleic acid molecule, or a RET fusion polypeptide encoded by the RET fusion nucleic acid molecule, wherein: (a) the RET fusion nucleic acid molecule comprises a fusion between a RET gene, or a portion thereof, and a gene listed in Table 1, or a portion thereof, or (b) the RET fusion nucleic acid molecule comprises a fusion between a RET gene, or a portion thereof, and a gene listed in Table 2, or a portion thereof, and the cancer is the cancer corresponding to the gene listed in Table 2, and wherein detection of the RET fusion nucleic acid molecule, or the RET fusion polypeptide encoded by the RET fusion nucleic acid molecule, in the sample identifies the individual as one who may benefit from a treatment comprising a RET-targeted therapy.

In another aspect, provided herein is a method of identifying one or more treatment options for an individual having a cancer, the method comprising: (a) detecting in a sample from the individual a RET fusion nucleic acid molecule, or a RET fusion polypeptide encoded by the RET fusion nucleic acid molecule, wherein: (i) the RET fusion nucleic acid molecule comprises a fusion between a RET gene, or a portion thereof, and a gene listed in Table 1, or a portion thereof, or (ii) the RET fusion nucleic acid molecule comprises a fusion between a RET gene, or a portion thereof, and a gene listed in Table 2, or a portion thereof, and the cancer is the cancer corresponding to the gene listed in Table 2; and (b) generating a report comprising one or more treatment options identified for the individual based at least in part on detection of the RET fusion nucleic acid molecule, or the RET fusion polypeptide encoded by the RET fusion nucleic acid molecule, in the sample, wherein the one or more treatment options comprise a RET-targeted therapy.

In another aspect, provided herein is method of identifying one or more treatment options for an individual having a cancer, the method comprising: (a) acquiring knowledge of a RET fusion nucleic acid molecule, or a RET fusion polypeptide encoded by the RET fusion nucleic acid molecule, in a sample from the individual, wherein: (i) the RET fusion nucleic acid molecule comprises a fusion between a RET gene, or a portion thereof, and a gene listed in Table 1, or a portion thereof, or (ii) the RET fusion nucleic acid molecule comprises a fusion between a RET gene, or a portion thereof, and a gene listed in Table 2, or a portion thereof, and the cancer is the cancer corresponding to the gene listed in Table 2; and (b) generating a report comprising one or more treatment options identified for the individual based at least in part on said knowledge, wherein the one or more treatment options comprise a RET-targeted therapy.

In another aspect, provided herein is a method of selecting a treatment for an individual having cancer, comprising acquiring knowledge of a RET fusion nucleic acid molecule, or a RET fusion polypeptide encoded by the RET fusion nucleic acid molecule, in a sample from the individual, wherein: (a) the RET fusion nucleic acid molecule comprises a fusion between a RET gene, or a portion thereof, and a gene listed in Table 1, or a portion thereof, or (b) the RET fusion nucleic acid molecule comprises a fusion between a RET gene, or a portion thereof, and a gene listed in Table 2, or a portion thereof, and the cancer is the cancer corresponding to the gene listed in Table 2, and wherein responsive to the acquisition of said knowledge: (i) the individual is classified as a candidate to receive a treatment comprising a RET-targeted therapy; and/or (ii) the individual is identified as likely to respond to a treatment that comprises a RET-targeted therapy.

In another aspect, provided herein is a method of predicting survival of an individual having a cancer, comprising acquiring knowledge of a RET fusion nucleic acid molecule, or a RET fusion polypeptide encoded by the RET fusion nucleic acid molecule, in a sample from the individual, wherein: (a) the RET fusion nucleic acid molecule comprises a fusion between a RET gene, or a portion thereof, and a gene listed in Table 1, or a portion thereof, or (b) the RET fusion nucleic acid molecule comprises a fusion between a RET gene, or a portion thereof, and a gene listed in Table 2, or a portion thereof, and the cancer is the cancer corresponding to the gene listed in Table 2, and wherein responsive to the acquisition of said knowledge, the individual is predicted to have longer survival when treated with a treatment comprising a RET-targeted therapy, as compared to survival of an individual whose cancer does not comprise a RET fusion nucleic acid molecule or a RET fusion polypeptide.

In another aspect, provided herein is a method of predicting survival of an individual having a cancer treated with a treatment comprising a RET-targeted therapy, the method comprising acquiring knowledge of a RET fusion nucleic acid molecule, or a RET fusion polypeptide encoded by the RET fusion nucleic acid molecule, in a sample from the individual, wherein: (a) the RET fusion nucleic acid molecule comprises a fusion between a RET gene, or a portion thereof, and a gene listed in Table 1, or a portion thereof, or (b) the RET fusion nucleic acid molecule comprises a fusion between a RET gene, or a portion thereof, and a gene listed in Table 2, or a portion thereof, and the cancer is the cancer corresponding to the gene listed in Table 2, and wherein responsive to the acquisition of said knowledge, the individual is predicted to have longer survival when treated with a treatment comprising a RET-targeted therapy, as compared to an individual whose cancer does not exhibit a RET fusion nucleic acid molecule or a RET fusion polypeptide.

In another aspect, provided herein is a method of treating or delaying progression of cancer in an individual, comprising: (a) acquiring knowledge of a RET fusion nucleic acid molecule, or a RET fusion polypeptide encoded by the RET fusion nucleic acid molecule, in a sample from the individual, wherein: (i) the RET fusion nucleic acid molecule comprises a fusion between a RET gene, or a portion thereof, and a gene listed in Table 1, or a portion thereof, or (ii) the RET fusion nucleic acid molecule comprises a fusion between a RET gene, or a portion thereof, and a gene listed in Table 2, or a portion thereof, and the cancer is the cancer corresponding to the gene listed in Table 2; and (b) responsive to said knowledge, administering to the individual an effective amount of a treatment that comprises a RET-targeted therapy.

In another aspect, provided herein is a method of treating or delaying progression of cancer, comprising administering to an individual having cancer an effective amount of a treatment that comprises a RET-targeted therapy, wherein the RET-targeted therapy is administered responsive to acquiring knowledge of a RET fusion nucleic acid molecule, or a RET fusion polypeptide encoded by the RET fusion nucleic acid molecule, in a sample from the individual, wherein: (a) the RET fusion nucleic acid molecule comprises a fusion between a RET gene, or a portion thereof, and a gene listed in Table 1, or a portion thereof, or (b) the RET fusion nucleic acid molecule comprises a fusion between a RET gene, or a portion thereof, and a gene listed in Table 2, or a portion thereof, and the cancer is the cancer corresponding to the gene listed in Table 2.

In another aspect, provided herein is a method of monitoring, evaluating or screening an individual having a cancer, comprising acquiring knowledge of a RET fusion nucleic acid molecule, or a RET fusion polypeptide encoded by the RET fusion nucleic acid molecule, in a sample from the individual, wherein: (a) the RET fusion nucleic acid molecule comprises a fusion between a RET gene, or a portion thereof, and a gene listed in Table 1, or a portion thereof, or (b) the RET fusion nucleic acid molecule comprises a fusion between a RET gene, or a portion thereof, and a gene listed in Table 2, or a portion thereof, and the cancer is the cancer corresponding to the gene listed in Table 2, and wherein responsive to the acquisition of said knowledge, the individual is predicted to have increased risk of cancer recurrence, aggressive cancer, anti-cancer therapy resistance, increased RET expression, clinical benefit from a RET-targeted therapy, or poor prognosis, as compared to an individual whose cancer does not comprise a RET fusion nucleic acid molecule or a RET fusion polypeptide. In some embodiments, responsive to the acquisition of said knowledge, the individual is predicted to have resistance to a non-RET-targeted anti-cancer therapy.

In another aspect, provided herein is a method of assessing a RET fusion nucleic acid molecule or a RET fusion polypeptide in a cancer in an individual, the method comprising: (a) detecting a RET fusion nucleic acid molecule, or a RET fusion polypeptide encoded by the RET fusion nucleic acid molecule, in a sample from the individual, wherein: (i) the RET fusion nucleic acid molecule comprises a fusion between a RET gene, or a portion thereof, and a gene listed in Table 1, or a portion thereof, or (ii) the RET fusion nucleic acid molecule comprises a fusion between a RET gene, or a portion thereof, and a gene listed in Table 2, or a portion thereof, and the cancer is the cancer corresponding to the gene listed in Table 2; and (b) providing an assessment of the RET fusion nucleic acid molecule, or the RET fusion polypeptide encoded by the RET fusion nucleic acid molecule, in the sample.

In another aspect, provided herein is a method of detecting a RET fusion nucleic acid molecule or a RET fusion polypeptide, the method comprising detecting in a sample from an individual having a cancer a RET fusion nucleic acid molecule, or a RET fusion polypeptide encoded by the RET fusion nucleic acid molecule, wherein: (a) the RET fusion nucleic acid molecule comprises a fusion between a RET gene, or a portion thereof, and a gene listed in Table 1, or a portion thereof, or (b) the RET fusion nucleic acid molecule comprises a fusion between a RET gene, or a portion thereof, and a gene listed in Table 2, or a portion thereof, and the cancer is the cancer corresponding to the gene listed in Table 2.

In another aspect, provided herein is a method of detecting the presence or absence of a cancer in an individual, the method comprising: (a) detecting the presence or absence of a cancer in a sample from the individual; and (b) detecting the presence or absence of a RET fusion nucleic acid molecule, or a RET fusion polypeptide encoded by the RET fusion nucleic acid molecule, in a sample from the individual, wherein: (i) the RET fusion nucleic acid molecule comprises a fusion between a RET gene, or a portion thereof, and a gene listed in Table 1, or a portion thereof, or (ii) the RET fusion nucleic acid molecule comprises a fusion between a RET gene, or a portion thereof, and a gene listed in Table 2, or a portion thereof, and the cancer is the cancer corresponding to the gene listed in Table 2. In some embodiments, the method comprises detecting the presence of the cancer in a sample from the individual. In some embodiments, the method comprises detecting the presence of the RET fusion nucleic acid molecule, or the RET fusion polypeptide encoded by the RET fusion nucleic acid molecule, in a sample from the individual.

In another aspect, provided herein is a method for monitoring progression or recurrence of a cancer in an individual, the method comprising: (a) detecting, in a first sample obtained from the individual at a first time point, the presence or absence of a RET fusion nucleic acid molecule, or a RET fusion polypeptide encoded by the RET fusion nucleic acid molecule; (b) detecting, in a second sample obtained from the individual at a second time point after the first time point, the presence or absence of a RET fusion nucleic acid molecule, or a RET fusion polypeptide encoded by the RET fusion nucleic acid molecule; and (c) providing an assessment of cancer progression or cancer recurrence in the individual based, at least in part, on the presence or absence of the RET fusion nucleic acid molecule, or the RET fusion polypeptide encoded by the RET fusion nucleic acid, in the first sample and/or in the second sample, wherein: (i) the RET fusion nucleic acid molecule comprises a fusion between a RET gene, or a portion thereof, and a gene listed in Table 1, or a portion thereof, or (ii) the RET fusion nucleic acid molecule comprises a fusion between a RET gene, or a portion thereof, and a gene listed in Table 2, or a portion thereof, and the cancer is the cancer corresponding to the gene listed in Table 2. In some embodiments, the presence of the RET fusion nucleic acid molecule, or the RET fusion polypeptide encoded by the RET fusion nucleic acid molecule, in the first sample and/or in the second sample identifies the individual as having increased risk of cancer progression or cancer recurrence. In some embodiments, the method further comprises selecting a treatment, administering a treatment, adjusting a treatment, adjusting a dose of a treatment, or applying a treatment to the individual based, at least in part, on detecting the presence of the RET fusion nucleic acid molecule, or the RET fusion polypeptide encoded by the RET fusion nucleic acid molecule, in the first sample and/or in the second sample, wherein the treatment comprises a RET-targeted therapy.

In another aspect, provided herein is a method of detecting a RET fusion nucleic acid molecule, the method comprising: (a) providing a plurality of nucleic acid molecules obtained from a sample from an individual having a cancer, wherein the plurality of nucleic acid molecules comprises nucleic acid molecules corresponding to: (i) a RET fusion nucleic acid molecule comprising a fusion between a RET gene, or a portion thereof, and a gene listed in Table 1, or a portion thereof, or (ii) a RET fusion nucleic acid molecule comprising a fusion between a RET gene, or a portion thereof, and a gene listed in Table 2, or a portion thereof, and the cancer is the cancer corresponding to the gene listed in Table 2; (b) optionally, ligating one or more adapters onto one or more nucleic acid molecules from the plurality of nucleic acid molecules; (c) optionally, amplifying the one or more ligated nucleic acid molecules from the plurality of nucleic acid molecules; (d) optionally, capturing amplified nucleic acid molecules from the amplified nucleic acid molecules; (e) sequencing, by a sequencer, the captured nucleic acid molecules to obtain a plurality of sequence reads that represent the captured nucleic acid molecules, wherein one or more of the plurality of sequence reads correspond to the RET fusion nucleic acid molecule; (f) analyzing the plurality of sequence reads; and (g) based on the analysis, detecting the presence or absence of the RET fusion nucleic acid molecule in the sample. In some embodiments, the method further comprises receiving, at one or more processors, sequence read data for the plurality of sequence reads. In some embodiments, the analyzing the plurality of sequence reads comprises identifying, using the one or more processors, the presence or absence of sequence reads corresponding to the RET fusion nucleic acid molecule. In some embodiments, the amplified nucleic acid molecules are captured by hybridization with one or more bait molecules.

In another aspect, provided herein is a method of detecting a RET fusion nucleic acid molecule, the method comprising: (a) providing a sample from an individual having a cancer, wherein the sample comprises a plurality of nucleic acid molecules; (b) preparing a nucleic acid sequencing library from the plurality of nucleic acid molecules in the sample; (c) amplifying said library; (d) selectively enriching for one or more nucleic acid molecules comprising nucleotide sequences corresponding to a RET fusion nucleic acid molecule in said library to produce an enriched sample, wherein: (i) the RET fusion nucleic acid molecule comprises a fusion between a RET gene, or a portion thereof, and a gene listed in Table 1, or a portion thereof, or (ii) the RET fusion nucleic acid molecule comprises a fusion between a RET gene, or a portion thereof, and a gene listed in Table 2, or a portion thereof, and the cancer is the cancer corresponding to the gene listed in Table 2; (e) sequencing the enriched sample, thereby producing a plurality of sequence reads; (f) analyzing the plurality of sequence reads for the presence of the RET fusion nucleic acid molecule; and (g) detecting, based on the analyzing step, the presence or absence of the RET fusion nucleic acid molecule in the sample from the individual.

In some embodiments, the plurality of nucleic acid molecules comprises a mixture of cancer nucleic acid molecules and non-cancer nucleic acid molecules. In some embodiments, the cancer nucleic acid molecules are derived from a tumor portion of a heterogeneous tissue biopsy sample, and the non-cancer nucleic acid molecules are derived from a normal portion of the heterogeneous tissue biopsy sample. In some embodiments, the sample comprises a liquid biopsy sample, and wherein the cancer nucleic acid molecules are derived from a circulating tumor DNA (ctDNA) fraction of the liquid biopsy sample, and the non-cancer nucleic acid molecules are derived from a non-tumor fraction of the liquid biopsy sample. In some embodiments, the one or more adapters comprise amplification primers, flow cell adapter sequences, substrate adapter sequences, sample index sequences, or unique molecular identifier (UMI) sequences. In some embodiments, the selectively enriching comprises: (a) combining one or more bait molecules with the library, thereby hybridizing the one or more bait molecules to one or more nucleic acid molecules comprising nucleotide sequences corresponding to the RET fusion nucleic acid molecule and producing nucleic acid hybrids; and (b) isolating the nucleic acid hybrids to produce the enriched sample. In some embodiments, the captured nucleic acid molecules are captured from the amplified nucleic acid molecules by hybridization to one or more bait molecules. In some embodiments, the amplifying comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencer comprises a next generation sequencer. In some embodiments, the method further comprises generating a molecular profile for the individual, based, at least in part, on detecting the presence or absence of the RET fusion nucleic acid molecule. In some embodiments, the molecular profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the molecular profile for the individual further comprises results from a nucleic acid sequencing-based test. In some embodiments, the method further comprises selecting a treatment, administering a treatment, or applying a treatment to the individual based on the generated molecular profile, wherein the treatment comprises a RET-targeted therapy. In some embodiments, the method further comprises generating a report indicating the presence or absence of the RET fusion nucleic acid molecule in the sample. In some embodiments, the method further comprises generating, by the one or more processors, a report indicating the presence or absence of the RET fusion nucleic acid molecule in the sample. In some embodiments, the method further comprises transmitting the report to the individual, a caregiver, a healthcare provider, a physician, an oncologist, an electronic medical record system, a hospital, a clinic, a third-party payer, an insurance company, or a government office. In some embodiments, the report is transmitted via a computer network or a peer-to-peer connection.

In another aspect, provided herein is a method of identifying a candidate treatment for a cancer in an individual in need thereof, comprising performing DNA sequencing on a sample obtained from the individual to determine a sequencing mutation profile, wherein the sequencing mutation profile identifies the presence or absence of a RET fusion nucleic acid molecule, wherein: (i) the RET fusion nucleic acid molecule comprises a fusion between a RET gene, or a portion thereof, and a gene listed in Table 1, or a portion thereof, or (ii) the RET fusion nucleic acid molecule comprises a fusion between a RET gene, or a portion thereof, and a gene listed in Table 2, or a portion thereof, and the cancer is the cancer corresponding to the gene listed in Table 2. In some embodiments, the candidate treatment comprises a RET-targeted therapy. In some embodiments, the presence of the RET fusion nucleic acid molecule in the sample identifies the individual as one who may benefit from a treatment comprising a RET-targeted therapy. In some embodiments, the presence of the RET fusion nucleic acid molecule in the sample predicts the individual to have longer survival when treated with a treatment comprising a RET-targeted therapy, as compared to survival of an individual whose cancer does not comprise a RET fusion nucleic acid molecule. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencing mutation profile identifies the presence or absence of a fragment of the RET fusion nucleic acid molecule comprising a breakpoint or fusion junction. In some embodiments, the fragment comprises any of at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more, nucleotides in length. In some embodiments, the fragment comprises between about 5 and about 100 nucleotides, between about 10 and about 50 nucleotides, or between about 10 and about 20 nucleotides, including any specific value within each of the recited ranges. In some embodiments, the fragment comprises any of at least about 1, 2, 3, 4, 5, 6, 7, 8, 9, or 10 nucleotides on either side of the breakpoint or fusion junction, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more, nucleotides on either side of the breakpoint or fusion junction.

In another aspect, provided herein is a method of treating or delaying progression of cancer, comprising: (a) detecting a RET fusion nucleic acid molecule, or a RET fusion polypeptide encoded by the RET fusion nucleic acid molecule, in a sample from an individual having a cancer, wherein: (i) the RET fusion nucleic acid molecule comprises a fusion between a RET gene, or a portion thereof, and a gene listed in Table 1, or a portion thereof, or (ii) the RET fusion nucleic acid molecule comprises a fusion between a RET gene, or a portion thereof, and a gene listed in Table 2, or a portion thereof, and the cancer is the cancer corresponding to the gene listed in Table 2; and (b) administering to the individual an effective amount of a treatment that comprises a RET-targeted therapy.

In some embodiments, which may be combined with any of the preceding aspects or embodiments, the RET fusion nucleic acid molecule is a RET fusion nucleic acid molecule listed in Table 3, and wherein the order of the genes in the fusion, in 5′ to 3′ direction, is as listed in Table 3. In some embodiments, which may be combined with any of the preceding aspects or embodiments, the RET fusion nucleic acid molecule is a RET fusion nucleic acid molecule listed in Table 4, and wherein the RET fusion nucleic acid molecule comprises or results from a corresponding 5″ breakpoint and/or 3′ breakpoint within the exons or introns as listed in Table 4. In some embodiments, which may be combined with any of the preceding aspects or embodiments, the RET fusion nucleic acid molecule is a RET fusion nucleic acid molecule listed in Table 5, and wherein the RET fusion nucleic acid molecule comprises or results from a corresponding 5′ breakpoint within the chromosomal coordinates as listed in Table 5, and/or a corresponding 3′ breakpoint within the chromosomal coordinates as listed in Table 5. In some embodiments, which may be combined with any of the preceding aspects or embodiments, the RET fusion nucleic acid molecule is a RET fusion nucleic acid molecule listed in Table 6, and wherein the RET fusion nucleic acid molecule comprises or results from a fusion of a corresponding 5′ exon as listed in Table 6, or a portion thereof, fused to a corresponding 3′ exon as listed in Table 6, or a portion thereof. In some embodiments, which may be combined with any of the preceding aspects or embodiments, the RET fusion nucleic acid molecule is a RET fusion nucleic acid molecule listed in Table 7, and wherein the RET fusion nucleic acid molecule comprises, in 5′ to 3′ direction, the corresponding exons or portions thereof as listed in Table 7. In some embodiments, which may be combined with any of the preceding aspects or embodiments, the RET fusion nucleic acid molecule is a RET fusion nucleic acid molecule listed in Table 8, and wherein the RET fusion nucleic acid molecule comprises a corresponding nucleotide sequence as listed in Table 8, or a nucleotide sequence with at least about 70% homology thereto. In some embodiments, which may be combined with any of the preceding aspects or embodiments, the RET fusion nucleic acid molecule encodes a RET fusion polypeptide. In some embodiments, which may be combined with any of the preceding aspects or embodiments, the RET fusion nucleic acid molecule comprises a nucleotide sequence encoding a corresponding RET fusion polypeptide as listed in Table 9, wherein the RET fusion polypeptide comprises a corresponding amino acid sequence as listed in Table 9, or an amino acid sequence with at least about 70% homology thereto. In some embodiments, which may be combined with any of the preceding aspects or embodiments, the RET fusion polypeptide encoded by the RET fusion nucleic acid molecule comprises a RET kinase domain, or a fragment of a RET kinase domain having RET kinase activity. In some embodiments, which may be combined with any of the preceding aspects or embodiments, the RET fusion polypeptide encoded by the RET fusion nucleic acid molecule has RET kinase activity. In some embodiments, which may be combined with any of the preceding aspects or embodiments, the RET fusion polypeptide encoded by the RET fusion nucleic acid molecule has a constitutive RET kinase activity. In some embodiments, which may be combined with any of the preceding aspects or embodiments, the RET fusion polypeptide encoded by the RET fusion nucleic acid molecule is oncogenic. In some embodiments, which may be combined with any of the preceding aspects or embodiments, the RET fusion polypeptide encoded by the RET fusion nucleic acid molecule promotes cancer cell survival, angiogenesis, cancer cell proliferation, and any combination thereof. In some embodiments, which may be combined with any of the preceding aspects or embodiments, the RET fusion polypeptide encoded by the RET fusion nucleic acid molecule is capable of dimerizing with a RET polypeptide or with another RET fusion polypeptide. In some embodiments, which may be combined with any of the preceding aspects or embodiments, the RET fusion polypeptide encoded by the RET fusion nucleic acid molecule is a RET fusion polypeptide listed in Table 9, and wherein the RET fusion polypeptide comprises a corresponding amino acid sequence as listed in Table 9, or an amino acid sequence with at least about 70% homology thereto.

In some embodiments, which may be combined with any of the preceding aspects or embodiments, the RET fusion nucleic acid molecule is a RET fusion nucleic acid molecule comprising a fusion between a RET gene, or a portion thereof, and a gene listed in Table 1, or a portion thereof, and wherein the cancer is a carcinoma, a sarcoma, a lymphoma, a leukemia, a myeloma, a germ cell cancer, or a blastoma. In some embodiments, which may be combined with any of the preceding aspects or embodiments, the RET fusion nucleic acid molecule is a RET fusion nucleic acid molecule comprising a fusion between a RET gene, or a portion thereof, and a gene listed in Table 1, or a portion thereof, and wherein the cancer is a solid tumor. In some embodiments, which may be combined with any of the preceding aspects or embodiments, the RET fusion nucleic acid molecule is a RET fusion nucleic acid molecule comprising a fusion between a RET gene, or a portion thereof, and a gene listed in Table 1, or a portion thereof, and wherein the cancer is a hematologic malignancy. In some embodiments, which may be combined with any of the preceding aspects or embodiments, the RET fusion nucleic acid molecule is a RET fusion nucleic acid molecule comprising a fusion between a RET gene, or a portion thereof, and a gene listed in Table 1, or a portion thereof, and wherein the cancer is a lymphoma.

In some embodiments, which may be combined with any of the preceding aspects or embodiments, the RET fusion nucleic acid molecule is a RET fusion nucleic acid molecule comprising a fusion between a RET gene, or a portion thereof, and a gene listed in Table 1, or a portion thereof, and wherein the cancer is: an ovarian cancer, a thyroid cancer, an adenocarcinoma, a breast cancer, a lung cancer, a colon cancer, a carcinoma, a uterine cancer, a prostate cancer, a pancreatic cancer, a leiomyosarcoma, a sarcoma, an esophageal cancer, a brain cancer, a bladder cancer, a skin cancer, a cervical cancer, or a melanoma; or ovary epithelial carcinoma, thyroid papillary carcinoma, unknown primary adenocarcinoma, breast carcinoma, lung non-small cell lung carcinoma, colon adenocarcinoma, unknown primary carcinoma, breast invasive ductal carcinoma, uterus endometrial adenocarcinoma mixed histology, prostate acinar adenocarcinoma, lung squamous cell carcinoma, lung small cell undifferentiated carcinoma, pancreas ductal adenocarcinoma, bladder urothelial (transitional cell) carcinoma, soft tissue leiomyosarcoma, soft tissue sarcoma, esophagus adenocarcinoma, ovary serous carcinoma, colon neuroendocrine carcinoma, brain glioblastoma, breast carcinoma, unknown primary malignant neoplasm, lung adenocarcinoma, unknown primary cancer, unknown primary serous carcinoma, thyroid carcinoma, uterus carcinosarcoma, pancreatobiliary carcinoma, unknown primary urothelial carcinoma, neuroendocrine tumor, unknown primary neuroendocrine tumor, brain astrocytoma, cholangiocarcinoma, intra-hepatic cholangiocarcinoma, cervix squamous cell carcinoma, or unknown primary melanoma. In some embodiments, which may be combined with any of the preceding aspects or embodiments, the RET fusion nucleic acid molecule is a RET fusion nucleic acid molecule comprising a fusion between a RET gene, or a portion thereof, and a gene listed in Table 1, or a portion thereof, and wherein the cancer is: a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, cpendymoma, pincaloma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypercosinophilic syndrome, systemic mastocytosis, familiar hypercosinophilia, chronic cosinophilic leukemia, neuroendocrine cancers, or a carcinoid tumor; or acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma. Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation. Merkel cell carcinoma, multicentric Castleman's disease, multiple hematologic malignancies including Philadelphia chromosome-positive ALL and CML, multiple myeloma, myclofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some embodiments, which may be combined with any of the preceding aspects or embodiments, the RET fusion nucleic acid molecule is a RET fusion nucleic acid molecule comprising a fusion between a RET gene, or a portion thereof, and a gene listed in Table 1, or a portion thereof, and wherein: (a) the RET fusion nucleic acid molecule is a RET fusion nucleic acid molecule as listed in Table 3, and wherein the order of the genes in the fusion, in 5′ to 3′ direction, is as listed in Table 3; (b) the RET fusion nucleic acid molecule is a RET fusion nucleic acid molecule as listed in Table 4, and wherein the RET fusion nucleic acid molecule comprises or results from a corresponding 5′ breakpoint and/or 3′ breakpoint within the exons or introns as listed in Table 4; (c) the RET fusion nucleic acid molecule is a RET fusion nucleic acid molecule as listed in Table 5, and wherein the RET fusion nucleic acid molecule comprises or results from a corresponding 5′ breakpoint within the chromosomal coordinates as listed in Table 5, and/or a corresponding 3′ breakpoint within the chromosomal coordinates as listed in Table 5; (d) the RET fusion nucleic acid molecule is a RET fusion nucleic acid molecule as listed in Table 6, and wherein the RET fusion nucleic acid molecule comprises or results from a fusion of a corresponding 5′ exon as listed in Table 6, or a portion thereof, fused to a corresponding 3′ exon as listed in Table 6, or a portion thereof; (e) the RET fusion nucleic acid molecule is a RET fusion nucleic acid molecule as listed in Table 7, and wherein the RET fusion nucleic acid molecule comprises, in 5′ to 3′ direction, the corresponding exons or portions thereof as listed in Table 7; (f) the RET fusion nucleic acid molecule is a RET fusion nucleic acid molecule as listed in Table 8, and wherein the RET fusion nucleic acid molecule comprises a corresponding nucleotide sequence as listed in Table 8, or a nucleotide sequence with at least about 70% homology thereto; and/or (g) the RET fusion nucleic acid molecule comprises a nucleotide sequence encoding a corresponding RET fusion polypeptide as listed in Table 9, wherein the RET fusion polypeptide comprises a corresponding amino acid sequence as listed in Table 9, or an amino acid sequence with at least about 70% homology thereto; and wherein the cancer is the corresponding cancer as listed in Table 10.

In some embodiments, which may be combined with any of the preceding aspects or embodiments, the cancer is metastatic.

In some embodiments, which may be combined with any of the preceding aspects or embodiments, the RET-targeted therapy comprises one or more of a small molecule inhibitor, an antibody, a cellular therapy, a nucleic acid, a virus-based therapy, an antibody-drug conjugate, a recombinant protein, a fusion protein, a natural compound, a peptide, a PROtcolysis-TArgeting Chimera (PROTAC), a treatment for RET-positive or RET-rearranged cancer, a RET-targeted therapy being tested in a clinical trial, a treatment for RET-positive or RET-rearranged cancer being tested in a clinical trial, or any combination thereof. In some embodiments, which may be combined with any of the preceding aspects or embodiments, the RET-targeted therapy is a kinase inhibitor. In some embodiments, the RET-targeted therapy is a tyrosine kinase inhibitor. In some embodiments, the RET-targeted therapy is kinase inhibitor that inhibits the kinase activity of a RET polypeptide. In some embodiments, the RET-targeted therapy is a multi-kinase inhibitor or a RET-specific inhibitor. In some embodiments, the RET-targeted therapy comprises one or more of pralsctinib, selpercatinib, lenvatinib, sorafenib, sunitinib, vandetanib, NVP-AST487, regorafenib, dovitinib, motesanib, cabozantinib, lapatinib, lestaurtinib, linifanib, semaxinib, ponatinib, fostamatinib, quizartinib, imatinib, vatalanib, ENMD-2076, JNJ-26483327, DCC-2157, Zetelctinib, TPX0046, TAS0953, RXDX-105, LOXO-260, BOS172738, Alectinib, APS03118, LOX-18228, or SYHA1815. In some embodiments, the nucleic acid inhibits the expression of the RET fusion nucleic acid molecule or the RET fusion polypeptide encoded by the RET fusion nucleic acid molecule. In some embodiments, the nucleic acid is a double-stranded RNA (dsRNA), a small interfering RNA (siRNA), or a small hairpin RNA (shRNA). In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In some embodiments, which may be combined with any of the preceding aspects or embodiments, the individual has received a prior anti-cancer treatment, or is being treated with an anti-cancer treatment. In some embodiments, the RET fusion nucleic acid molecule, and/or the RET fusion polypeptide encoded by the RET fusion nucleic acid molecule, confers resistance of the cancer to the anti-cancer treatment. In some embodiments, the anti-cancer treatment is a small molecule inhibitor, an antibody, a cellular therapy, a nucleic acid, a virus-based therapy, an antibody-drug conjugate, a recombinant protein, a fusion protein, a natural compound, a peptide, a PROtcolysis-TArgeting Chimera (PROTAC), a treatment for cancer being tested in a clinical trial, an immunotherapy, a chemotherapy, a targeted therapy, a non-RET-targeted anti-cancer therapy, or any combination thereof. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the nucleic acid comprises a double-stranded RNA (dsRNA), a small interfering RNA (siRNA), or a small hairpin RNA (shRNA).

In some embodiments, which may be combined with any of the preceding aspects or embodiments, the cancer has not been previously treated. In some embodiments, which may be combined with any of the preceding aspects or embodiments, the RET-targeted therapy is a first-line or front-line treatment. In some embodiments, which may be combined with any of the preceding aspects or embodiments, the RET-targeted therapy comprises one or more of pralsctinib, selpercatinib, lenvatinib, sorafenib, sunitinib, vandetanib, NVP-AST487, regorafenib, dovitinib, motesanib, cabozantinib, lapatinib, lestaurtinib, linifanib, semaxinib, ponatinib, fostamatinib, quizartinib, imatinib, vatalanib, ENMD-2076, JNJ-26483327, DCC-2157, Zetelctinib, TPX0046, TAS0953, RXDX-105, LOXO-260, BOS172738, Alectinib, APS03118, LOX-18228, or SYHA1815.

In some embodiments, which may be combined with any of the preceding aspects or embodiments, the cancer is kinase inhibitor-naïve. In some embodiments, which may be combined with any of the preceding aspects or embodiments, the cancer has not been previously treated with a kinase inhibitor.

In some embodiments, which may be combined with any of the preceding aspects or embodiments, the cancer has been previously treated with a kinase inhibitor. In some embodiments, which may be combined with any of the preceding aspects or embodiments, the cancer progressed on a prior treatment with a kinase inhibitor. In some embodiments, which may be combined with any of the preceding aspects or embodiments, the cancer is refractory to a prior kinase inhibitor treatment. In some embodiments, which may be combined with any of the preceding aspects or embodiments, the cancer progressed on a prior treatment with a chemotherapy and a kinase inhibitor.

In some embodiments, which may be combined with any of the preceding aspects or embodiments, the kinase inhibitor is a tyrosine kinase inhibitor. In some embodiments, the kinase inhibitor inhibits the kinase activity of a RET polypeptide. In some embodiments, the kinase inhibitor is a multi-kinase inhibitor or a RET-specific inhibitor. In some embodiments, the RET-targeted therapy comprises one or more of pralsetinib, selpercatinib, lenvatinib, sorafenib, sunitinib, vandetanib, NVP-AST487, regorafenib, dovitinib, motesanib, cabozantinib, lapatinib, lestaurtinib, linifanib, semaxinib, ponatinib, fostamatinib, quizartinib, imatinib, vatalanib, ENMD-2076, JNJ-26483327, DCC-2157, Zetelctinib, TPX0046, TAS0953, RXDX-105, LOXO-260, BOS172738, Alcctinib, APS03118, LOX-18228, or SYHA1815.

In some embodiments, which may be combined with any of the preceding aspects or embodiments, the treatment or the one or more treatment options further comprise an additional anti-cancer therapy. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROtcolysis-TArgeting Chimera (PROTAC), or any combination thereof. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the nucleic acid comprises a double-stranded RNA (dsRNA), a small interfering RNA (siRNA), or a small hairpin RNA (shRNA).

In some embodiments, which may be combined with any of the preceding aspects or embodiments, the method further comprises obtaining the sample from the individual. In some embodiments, which may be combined with any of the preceding aspects or embodiments, the sample is obtained from the cancer. In some embodiments, which may be combined with any of the preceding aspects or embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, cerebrospinal fluid, sputum, stool, urine, or saliva. In some embodiments, which may be combined with any of the preceding aspects or embodiments, the sample comprises cells and/or nucleic acids from the cancer. In some embodiments, the sample comprises mRNA, DNA, circulating tumor DNA (ctDNA), cell-free DNA, or cell-free RNA from the cancer. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof.

In some embodiments, which may be combined with any of the preceding aspects or embodiments, the method comprises acquiring knowledge of or detecting the RET fusion nucleic acid molecule or the RET fusion polypeptide encoded by the RET fusion nucleic acid molecule in a tissue biopsy sample, in a liquid biopsy sample, or in both a tissue biopsy sample and a liquid biopsy sample, from the individual.

In some embodiments, which may be combined with any of the preceding aspects or embodiments, the acquiring knowledge of the RET fusion nucleic acid molecule or the RET fusion polypeptide encoded by the RET fusion nucleic acid molecule comprises detecting the RET fusion nucleic acid molecule, or the RET fusion polypeptide encoded by the RET fusion nucleic acid molecule, in the sample. In some embodiments, which may be combined with any of the preceding aspects or embodiments, detecting the RET fusion nucleic acid molecule in the sample comprises detecting: (i) a fragment of the RET fusion nucleic acid molecule comprising a breakpoint or fusion junction between the RET gene, or the portion thereof, and the gene listed in Table 1, or the portion thereof, or (ii) a fragment of the RET fusion nucleic acid molecule comprising a breakpoint or fusion junction between the RET gene, or the portion thereof, and the gene listed in Table 2, or the portion thereof. In some embodiments, the fragment comprises any of at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more, nucleotides in length. In some embodiments, the fragment comprises between about 5 and about 100 nucleotides, between about 10 and about 50 nucleotides, or between about 10 and about 20 nucleotides, including any specific value within each of the recited ranges. In some embodiments, the fragment comprises any of at least about 1, 2, 3, 4, 5, 6, 7, 8, 9, or 10 nucleotides on either side of the breakpoint or fusion junction, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more, nucleotides on either side of the breakpoint or fusion junction. In some embodiments, which may be combined with any of the preceding aspects or embodiments, the RET fusion nucleic acid molecule is detected in the sample by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing. In some embodiments, the sequencing comprises a massively parallel sequencing (MPS) technique, whole genome sequencing (WGS), whole exome sequencing, targeted sequencing, direct sequencing, or a Sanger sequencing technique; and optionally wherein the massively parallel sequencing (MPS) technique comprises next-generation sequencing (NGS). In some embodiments, which may be combined with any of the preceding aspects or embodiments, detecting the RET fusion polypeptide encoded by the RET fusion nucleic acid molecule comprises detecting a fragment of the RET fusion polypeptide that is encoded by: (i) a fragment of the RET fusion nucleic acid molecule that comprises a breakpoint or fusion junction between the RET gene, or the portion thereof, and the gene listed in Table 1, or the portion thereof, or (ii) a fragment of the RET fusion nucleic acid molecule that comprises a breakpoint or fusion junction between the RET gene, or the portion thereof, and the gene listed in Table 2, or the portion thereof. In some embodiments, the fragment comprises any of at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more, nucleotides in length. In some embodiments, the fragment comprises between about 5 and about 100 nucleotides, between about 10 and about 50 nucleotides, or between about 10 and about 20 nucleotides, including any specific value within each of the recited ranges. In some embodiments, the fragment comprises any of at least about 1, 2, 3, 4, 5, 6, 7, 8, 9, or 10 nucleotides on either side of the breakpoint or fusion junction, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more, nucleotides on either side of the breakpoint or fusion junction. In some embodiments, the RET fusion polypeptide encoded by the RET fusion nucleic acid molecule is detected in the sample by one or more of: immunoblotting, enzyme linked immunosorbent assay (ELISA), immunohistochemistry, or mass spectrometry.

In some embodiments, which may be combined with any of the preceding aspects or embodiments, the method further comprises selectively enriching for one or more nucleic acid molecules in the sample comprising nucleotide sequences corresponding to the RET fusion nucleic acid molecule; wherein the selectively enriching produces an enriched sample. In some embodiments, the selectively enriching comprises: (a) combining one or more bait molecules with the sample, thereby hybridizing the one or more bait molecules to one or more nucleic acids in the sample comprising nucleotide sequences corresponding to the RET fusion nucleic acid molecule and producing nucleic acid hybrids; and (b) isolating the nucleic acid hybrids to produce the enriched sample. In some embodiments, which may be combined with any of the preceding aspects or embodiments, the one or more bait molecules comprise a capture nucleic acid molecule configured to hybridize to a nucleotide sequence corresponding to the RET fusion nucleic acid molecule. In some embodiments, the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides. In some embodiments, which may be combined with any of the preceding aspects or embodiments, the one or more bait molecules are conjugated to an affinity reagent or to a detection reagent. In some embodiments, the affinity reagent is an antibody, an antibody fragment, or biotin, or wherein the detection reagent is a fluorescent marker. In some embodiments, which may be combined with any of the preceding aspects or embodiments, the capture nucleic acid molecule comprises a DNA, RNA, or mixed DNA/RNA molecule. In some embodiments, which may be combined with any of the preceding aspects or embodiments, the selectively enriching comprises amplifying the one or more nucleic acid molecules comprising nucleotide sequences corresponding to the RET fusion nucleic acid molecule using a polymerase chain reaction (PCR) to produce an enriched sample. In some embodiments, which may be combined with any of the preceding aspects or embodiments, the method further comprises sequencing the enriched sample. In some embodiments, which may be combined with any of the preceding aspects or embodiments, the method further comprises acquiring knowledge of or detecting in a sample from the individual a base substitution, a short insertion/deletion (indel), a copy number alteration, or a genomic rearrangement in one or more genes.

In some embodiments, which may be combined with any of the preceding aspects or embodiments, the individual is a human.

In another aspect, provided herein is a kit comprising one or more probes, baits, and/or oligonucleotides for detecting: (i) a RET fusion nucleic acid molecule comprising a fusion between a RET gene, or a portion thereof, and a gene listed in Table 1, or a portion thereof, or (ii) a RET fusion nucleic acid molecule comprising a fusion between a RET gene, or a portion thereof, and a gene listed in Table 2, or a portion thereof, in a corresponding cancer as listed in Table 2.

In another aspect, provided herein is a nucleic acid, such as an isolated nucleic acid, encoding a RET fusion nucleic acid molecule, or a fragment thereof, comprising a fusion between a RET gene, or a portion thereof, and a gene listed in Table 1, or a portion thereof. In another aspect, provided herein is a vector comprising a nucleic acid provided herein. In another aspect, provided herein is a host cell comprising a vector provided herein.

In another aspect, provided herein is an antibody or antibody fragment that specifically binds to a RET fusion polypeptide, or to a portion thereof, wherein the RET fusion polypeptide is encoded by a RET fusion nucleic acid molecule comprising a fusion between a RET gene, or a portion thereof, and a gene listed in Table 1, or a portion thereof.

In another aspect, provided herein is a kit comprising an antibody or antibody fragment provided herein.

In another aspect, provided herein is an in vitro use of one or more probes, baits, and/or oligonucleotides for detecting: (i) a RET fusion nucleic acid molecule comprising a fusion between a RET gene, or a portion thereof, and a gene listed in Table 1, or a portion thereof, or (ii) a RET fusion nucleic acid molecule comprising a fusion between a RET gene, or a portion thereof, and a gene listed in Table 2, or a portion thereof, in a corresponding cancer as listed in Table 2.

In another aspect, provided herein is a system, comprising: a memory configured to store one or more program instructions, and one or more processors configured to execute the one or more program instructions, the one or more program instructions when executed by the one or more processors are configured to: (a) obtain a plurality of sequence reads of one or more nucleic acid molecules, wherein the one or more nucleic acid molecules are derived from a sample obtained from an individual having a cancer; (b) analyze the plurality of sequence reads for the presence of a RET fusion nucleic acid molecule, wherein: (i) the RET fusion nucleic acid molecule comprises a fusion between a RET gene, or a portion thereof, and a gene listed in Table 1, or a portion thereof, or (ii) the RET fusion nucleic acid molecule comprises a fusion between a RET gene, or a portion thereof, and a gene listed in Table 2, or a portion thereof, and wherein the cancer is the corresponding cancer as listed in Table 2; and (c) detect, based on the analyzing, the RET fusion nucleic acid molecule in the sample.

In another aspect, provided herein is a non-transitory computer readable storage medium comprising one or more programs executable by one or more computer processors for performing a method, the method comprising: (a) obtaining, using the one or more processors, a plurality of sequence reads of one or more nucleic acid molecules, wherein the one or more nucleic acid molecules are derived from a sample obtained from an individual having a cancer; (b) analyzing, using the one or more processors, the plurality of sequence reads for the presence of a RET fusion nucleic acid molecule, wherein: (i) the RET fusion nucleic acid molecule comprises a fusion between a RET gene, or a portion thereof, and a gene listed in Table 1, or a portion thereof, or (ii) the RET fusion nucleic acid molecule comprises a fusion between a RET gene, or a portion thereof, and a gene listed in Table 2, or a portion thereof, and wherein the cancer is the corresponding cancer as listed in Table 2; and (c) detecting, using the one or more processors and based on the analyzing, the RET fusion nucleic acid molecule in the sample.

In some embodiments, which may be combined with any of the preceding aspects or embodiments, the plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next generation sequencing (NGS). In some embodiments, which may be combined with any of the preceding aspects or embodiments, the one or more program instructions when executed by the one or more processors are further configured to generate, based at least in part on the detecting, a molecular profile for the sample. In some embodiments, which may be combined with any of the preceding aspects or embodiments, the method further comprises generating, based at least in part on the detecting, a molecular profile for the sample. In some embodiments, which may be combined with any of the preceding aspects or embodiments, the individual is administered a treatment based at least in part on the molecular profile; optionally wherein the treatment comprises a RET-targeted therapy. In some embodiments, which may be combined with any of the preceding aspects or embodiments, the molecular profile further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, which may be combined with any of the preceding aspects or embodiments, the molecular profile further comprises results from a nucleic acid sequencing-based test.

In another aspect, provided herein is a RET-targeted therapy for use in a method of treating or delaying progression of cancer, wherein the method comprises administering the RET-targeted therapy to an individual having a cancer, wherein a RET fusion nucleic acid molecule, or a RET fusion polypeptide encoded by the RET fusion nucleic acid molecule, is detected in a sample from the individual, and wherein: (a) the RET fusion nucleic acid molecule comprises a fusion between a RET gene, or a portion thereof, and a gene listed in Table 1, or a portion thereof, or (b) the RET fusion nucleic acid molecule comprises a fusion between a RET gene, or a portion thereof, and a gene listed in Table 2, or a portion thereof, and wherein the cancer is the corresponding cancer as listed in Table 2.

In another aspect, provided herein is a RET-targeted therapy for use in the manufacture of a medicament for treating or delaying progression of cancer, wherein the medicament is to be administered to an individual having a cancer, wherein a RET fusion nucleic acid molecule, or a RET fusion polypeptide encoded by the RET fusion nucleic acid molecule, is detected in a sample from the individual, and wherein: (a) the RET fusion nucleic acid molecule comprises a fusion between a RET gene, or a portion thereof, and a gene listed in Table 1, or a portion thereof, or (b) the RET fusion nucleic acid molecule comprises a fusion between a RET gene, or a portion thereof, and a gene listed in Table 2, or a portion thereof, and wherein the cancer is the corresponding cancer as listed in Table 2.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 depicts an exemplary device, in accordance with some embodiments.

FIG. 2 depicts an exemplary system, in accordance with some embodiments.

FIG. 3 depicts a block diagram of an exemplary process for detecting a RET fusion nucleic acid molecule, in accordance with some embodiments.

FIGS. 4A-4C depict the prevalence of RET fusions in various tumor types in the study described in Example 2, herein. FIG. 4A is a bar graph showing the prevalence of RET fusions in eight tumor categories. Only tumor types in which there were more than 10 RET fusion instances are shown. FIG. 4B is a bar graph showing the prevalence of RET fusions in non-small cell lung cancer (NSCLC) subtypes. FIG. 4C is a bar graph showing the prevalence of RET fusions in thyroid carcinoma subtypes. In FIGS. 4A-4C, the number on top of each bar indicates the total number of RET fusion cases in the respective tumor type.

FIGS. 5A-5B depict the distribution of RET gene breakpoint regions in the NSCLC and non-NSCLC RET fusion-positive cohorts of the study described in Example 2, herein. FIG. 5A is a representative lollipop plot scheme of the RET gene [Chr10 (10q11.21)], showing the frequency of RET gene fusion breakpoints among advanced RET fusion-positive NSCLC and other solid tumors (non-NSCLC). FIG. 5B is a representative lollipop plot scheme of the RET gene [Chr10 (10q11.21)] showing the frequency of RET gene fusion breakpoints among advanced RET fusion-positive NSCLC and other solid tumors (non-NSCLC) in liquid biopsy samples. In FIGS. 5A-5B, the dark horizontal lines indicate RET gene introns. Vertical bars indicate RET gene exons. Coding region extending from 43,077,259 to 43,128,266. Lollipops indicate prevalence of RET breakpoints binned by 100 bases. The RET extracellular region is coded by exons 1-10 and part of exon 11 (amino acids 29 to 635), responsible for the cadherin-like 1 (CLD 1), CLD 2, CLD 3, CLD 4, and cysteine-rich (CRD) domains (responsible for physiological receptor dimerization). A transmembrane region (TM) is coded by part of exon 11 (amino acids 636-657). Bipartite protein tyrosine kinase domains are coded by part of exon 12, exons 13-18 and part of exon 19 (amino acids 658 to 1114). Tyr K, cytoplasmic intrinsic tyrosine kinase domain.

FIGS. 6A-6D depict the prevalence of genes with genomic alterations among RET fusion-defined cohorts in the study described in Example 2, herein. FIG. 6A is a plot indicating the prevalence of concurrent genomic variants among advanced RET fusion-positive NSCLC and RET fusion-negative NSCLC cases. Significant differences in the prevalence of genes with genomic alterations between RET fusion-positive NSCLC and RET fusion-negative NSCLC are indicated by patterned (p≤0.05) or solid dots (p≤0.0001). Off scale arrows indicate that while the actual prevalence of a gene is not shown in the figure, its prevalence is in the direction of the arrow. FIG. 6B depicts the prevalence of the five most common genes with genomic alterations in advanced RET fusion-positive thyroid cancers. FIG. 6C depicts the prevalence of the five most common genes with genomic alterations among advanced RET fusion-positive colon carcinomas, pancreatic carcinomas, breast carcinomas, and unknown primary carcinomas. FIG. 6D depicts the prevalence of the five most common genes with genomic alterations among advanced RET fusion-positive brain tumors, salivary gland carcinomas, ovarian carcinomas, and cholangiocarcinomas.

### DETAILED DESCRIPTION

The present disclosure relates generally to detecting RET gene fusions in cancer, as well as methods of treatment, and uses related thereto.

Kinase fusions are an important class of targetable oncogenic driver variants. The present disclosure describes the results of comprehensive genomic profiling of the pan-cancer landscape of RET gene fusions. These analyses identified diverse rearrangements leading to fusion genes involving RET and numerous fusion partner genes (see, e.g., Examples 1-2). Without wishing to be bound by theory, it is thought that the presence of a RET fusion described herein in a sample from individuals having cancer may identify cancer patients who are likely to respond to treatment with an anti-cancer therapy such as a targeted anti-cancer therapy, e.g., a RET-targeted therapy as described herein.

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al, eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. De Vita et al., eds., J. B. Lippincott Company, 1993).

### II. Definitions

As used in this specification and the appended claims, the singular forms “a”, “an” and “the” include plural referents unless the context clearly dictates otherwise. Thus, for example, reference to “a molecule” optionally includes a combination of two or more such molecules, and the like.

The terms “about” and “approximately” as used herein refer to the usual error range for the respective value readily known to the skilled person in this technical field. Exemplary degrees of error are within 20 percent (%), typically, within 10%, and more typically, within 5% of a given value or range of values. Reference to “about” or “approximately” a value or parameter herein includes (and describes) embodiments that are directed to that value or parameter per se.

It is understood that aspects and embodiments of the invention described herein include “comprising.” “consisting.” and “consisting essentially of” aspects and embodiments.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

“Polynucleotide,” “nucleic acid,” or “nucleic acid molecule” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro-, or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(0)S (“thioate”), P(S)S (“dithioate”), “(0)NR₂ (“amidate”), P(0)R, P(0)OR′, CO or CH₂ (“formacetal”), in which each R or R′ is independently H or substituted or unsubstituted alkyl (1-20 C) optionally containing an ether (-0-) linkage, aryl, alkenyl, cycloalkyl, cycloalkenyl or araldyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “isolated” antibody is one which has been identified and separated and/or recovered from a component of its natural environment. Contaminant components of its natural environment are materials which would interfere with research, diagnostic, and/or therapeutic uses for the antibody, and may include enzymes, hormones, and other proteinaceous or nonproteinaccous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. An isolated antibody includes the antibody in situ within recombinant cells since at least one component of the antibody's natural environment will not be present. Ordinarily, however, an isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The “light chains” of antibodies (immunoglobulins) from any mammalian species can be assigned to one of two clearly distinct types, called kappa (“κ”) and lambda (“λ”), based on the amino acid sequences of their constant domains.

The term “constant domain” refers to the portion of an immunoglobulin molecule having a more conserved amino acid sequence relative to the other portion of the immunoglobulin, the variable domain, which contains the antigen binding site. The constant domain contains the CH1, CH2, and CH3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The term “hypervariable region,” “HVR,” or “HV,” as used herein, refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, for example, Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, for example, Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat |
| | | | | numbering) |
| H1 | H31-H35 | H26-H35 | H26-H32 | H30-H35 (Chothia |
| | | | | numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc, according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-1 13 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human lgG1 EU antibody.

The terms “full-length antibody,” “intact antibody.” and “whole antibody” are used herein interchangeably to refer to an antibody in its substantially intact form, not antibody fragments as defined below. The terms particularly refer to an antibody with heavy chains that contain an Fc region.

“Antibody fragments” comprise a portion of an intact antibody comprising the antigen-binding region thereof. In some embodiments, the antibody fragment described herein is an antigen-binding fragment. Examples of antibody fragments include Fab, Fab′, F(ab′)2, and Fv fragments; diabodies; linear antibodies; single-chain antibody molecules; and multispecific antibodies formed from antibody fragments.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target-binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target-binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target-binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature 256:495-97 (1975); Hongo et al., Hybridoma 14 (3): 253-260 (1995), Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352:624-628 (1991); Marks et al., J. Mol. Biol. 222:581-597 (1992); Sidhu et al., J. Mol. Biol. 338 (2): 299-310 (2004); Lee et al., J. Mol. Biol. 340 (5): 1073-1093 (2004); Fellouse, Proc. Natl. Acad. Sci. USA 101 (34): 12467-12472 (2004); and Lee et al., J. Immunol. Methods 284 (1-2): 119-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg et al., Intern. Rev. Immunol. 13:65-93 (1995)).

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human framework regions (FRs). In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody.

A “humanized form” of an antibody, e.g., a non-human antibody, refers to an antibody that has undergone humanization.

A “blocking” antibody or an “antagonist” antibody is one which inhibits or reduces biological activity of the antigen it binds. For example, blocking antibodies or antagonist antibodies substantially or completely inhibit the biological activity of the antigen.

As used herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of <1 μM, <100 nM, <10 nM, <1 nM, or <0.1 nM. In certain embodiments, an antibody specifically binds to an epitope on a protein that is conserved among the protein from different species. In another embodiment, specific binding can include, but does not require exclusive binding.

The terms “homology” or “identity.” as used herein, refer to sequence similarity between two polynucleotide sequences or between two polypeptide sequences. The phrases “percent identity or homology” and “% identity or homology” refer to the percentage of sequence similarity found in a comparison of two or more polynucleotide sequences or two or more polypeptide sequences. Identity or similarity can be determined by comparing a position in each sequence that can be aligned for purposes of comparison. When a position in the compared sequences is occupied by the same nucleotide base or amino acid, then the molecules are identical at that position.

The term “detection” includes any means of detecting, including direct and indirect detection. The term “biomarker” as used herein (e.g., a “biomarker” such as a RET fusion or a RET fusion nucleic acid molecule or polypeptide described herein) refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features (e.g., responsiveness to therapy). In some embodiments, a biomarker is a collection of genes or a collective number of mutations/alterations (e.g., somatic mutations) in a collection of genes. Biomarkers include, but are not limited to, polynucleotides (e.g., DNA and/or RNA), polynucleotide alterations (e.g., polynucleotide copy number alterations, e.g., DNA copy number alterations), polypeptides, polypeptide and polynucleotide modifications (e.g., post-translational modifications), carbohydrates, and/or glycolipid-based molecular markers.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

The term “diagnosis” is used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes)).

The term “aiding diagnosis” is used herein to refer to methods that assist in making a clinical determination regarding the presence, or nature, of a particular type of symptom or condition of a disease or disorder (e.g., cancer). For example, a method of aiding diagnosis of a disease or condition (e.g., cancer) can comprise measuring certain somatic mutations in a biological sample from an individual.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof. In some instances, the sample is a whole blood sample, a plasma sample, a serum sample, or a combination thereof. In some embodiments, the sample is from a tumor (e.g., a “tumor sample”), such as from a biopsy. In some embodiments, the sample is a formalin-fixed paraffin-embedded (FFPE) sample.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” or “control tissue,” as used herein, refer to a sample, cell, tissue, standard, or level that is used for comparison purposes.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

An “effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and in some embodiments stop) cancer cell infiltration into peripheral organs; inhibit (i.e., slow to some extent and in some embodiments stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), response rates (e.g., CR and PR), duration of response, and/or quality of life.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the patient herein is a human.

As used herein, “administering” is meant a method of giving a dosage of an agent or a pharmaceutical composition (e.g., a pharmaceutical composition including the agent) to a subject (e.g., a patient). Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The term “concurrently.” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

The term “package insert” is used to refer to instructions customarily included in commercial packages of therapeutic products, that contain information about the indications, usage, dosage, administration, combination therapy, contraindications, and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a reagent for specifically detecting a biomarker (e.g., a RET fusion nucleic acid molecule or polypeptide described herein) described herein. In certain embodiments, the manufacture or kit is promoted, distributed, or sold as a unit for performing the methods described herein.

The phrase “based on”, “responsive to”, and the like, when used herein mean that the information about one or more biomarkers (e.g., a RET fusion or a RET fusion nucleic acid molecule or polypeptide described herein) is used to inform a treatment decision, information provided on a package insert, or marketing/promotional guidance, etc.

The terms “allele frequency” and “allele fraction” are used interchangeably herein and refer to the fraction of sequence reads corresponding to a particular allele relative to the total number of sequence reads for a genomic locus. The terms “variant allele frequency” and “variant allele fraction” are used interchangeably herein and refer to the fraction of sequence reads corresponding to a particular variant allele relative to the total number of sequence reads for a genomic locus.

### III. Methods, Systems, and Devices

In some aspects, provided herein are methods for identifying an individual having a cancer who may benefit from a treatment comprising a rearranged during transfection (RET)-targeted therapy. In other aspects, provided herein are methods for selecting a therapy or treatment for an individual having a cancer. In other aspects, provided herein are methods for identifying one or more treatment options for an individual having a cancer. In other aspects, provided herein are methods for predicting survival of an individual having a cancer. In other aspects, provided herein are methods for predicting survival of an individual having a cancer treated with a treatment comprising a RET-targeted therapy. In other aspects, provided herein are methods for treating or delaying progression of cancer. In other aspects, provided herein are methods for monitoring, evaluating or screening an individual having a cancer. In other aspects, provided herein are methods for assessing a RET fusion nucleic acid molecule or polypeptide in a cancer in an individual. In other aspects, provided herein are methods for detecting a RET fusion nucleic acid molecule or polypeptide in a sample from an individual having a cancer. In other aspects, provided herein are methods for detecting the presence or absence of a cancer and/or a RET fusion nucleic acid molecule or polypeptide in an individual. In other aspects, provided herein are methods for monitoring progression or recurrence of a cancer in an individual.

In some embodiments of any of the methods provided herein, the methods comprise detecting the presence or absence of a RET fusion nucleic acid molecule provided herein, or a fragment thereof, in a sample from an individual. In other embodiments of any of the methods provided herein, the methods comprise detecting the presence or absence of a RET fusion polypeptide provided herein, or a fragment thereof, in a sample from an individual. In other embodiments of any of the methods provided herein, the methods comprise acquiring knowledge of the presence or absence of a RET fusion polypeptide provided herein, or a fragment thereof, in a sample from an individual. In other embodiments of any of the methods provided herein, the methods comprise acquiring knowledge of the presence or absence of a RET fusion nucleic acid molecule provided herein, or a fragment thereof, in a sample from an individual. In some embodiments, detection of a RET fusion nucleic acid molecule or polypeptide of the disclosure, or a fragment thereof, in the sample identifies the individual as one who may benefit from a treatment comprising a RET-targeted therapy. In some embodiments, the methods further comprise generating a report comprising one or more treatment options identified for the individual based at least in part on detection of the RET fusion nucleic acid molecule or polypeptide, or a fragment thereof, in the sample, wherein the one or more treatment options comprise a RET-targeted therapy. In some embodiments, the methods further comprise generating a report comprising one or more treatment options identified for the individual based at least in part on knowledge of the presence of the RET fusion nucleic acid molecule or polypeptide, or a fragment thereof, in a sample from an individual, wherein the one or more treatment options comprise a RET-targeted therapy. In some embodiments, responsive to the acquisition of knowledge of the presence of the RET fusion nucleic acid molecule or polypeptide, or a fragment thereof, in a sample from an individual: (i) the individual is classified as a candidate to receive a treatment comprising a RET-targeted therapy; and/or (ii) the individual is identified as likely to respond to a treatment that comprises a RET-targeted therapy. In some embodiments, responsive to the acquisition of knowledge of the presence of the RET fusion nucleic acid molecule or polypeptide, or a fragment thereof, in a sample from an individual, the individual is predicted to have longer survival when treated with a treatment comprising a RET-targeted therapy, as compared to survival of an individual whose cancer does not comprise a RET fusion nucleic acid molecule or polypeptide. In some embodiments, responsive to the acquisition of knowledge of the presence of the RET fusion nucleic acid molecule or polypeptide, or a fragment thereof, in a sample from an individual, the method comprises administering to the individual an effective amount of a treatment that comprises a RET-targeted therapy. In some embodiments, responsive to the acquisition of knowledge of the presence of the RET fusion nucleic acid molecule or polypeptide, or a fragment thereof, in a sample from an individual, the individual is predicted to have increased risk of cancer recurrence, aggressive cancer, resistance to an anti-cancer therapy, e.g., a non-RET-targeted therapy, poor prognosis, e.g., when treated with a non-RET-targeted therapy, increased expression of RET, or clinical benefit to RET-targeted therapies, as compared to an individual whose cancer does not comprise a RET fusion nucleic acid molecule or polypeptide. In some embodiments, the methods provided herein comprise providing an assessment of the RET fusion nucleic acid molecule or polypeptide, or fragment thereof, e.g., in an individual or in a sample from an individual. In some embodiments, the methods provided herein comprise detecting the RET fusion nucleic acid molecule or polypeptide, or a fragment thereof, in a sample from an individual, and administering to the individual an effective amount of a treatment that comprises a RET-targeted therapy. In some embodiments, the methods provided herein comprise acquiring knowledge of the presence of the RET fusion nucleic acid molecule or polypeptide, or a fragment thereof, in a sample from an individual, and administering to the individual an effective amount of a treatment that comprises a RET-targeted therapy.

In other aspects, provided herein are systems and non-transitory computer readable storage media. In some embodiments, a system of the disclosure comprises a memory configured to store one or more program instructions; and one or more processors configured to execute the one or more program instructions, the one or more program instructions when executed by the one or more processors are configured to: (a) obtain a plurality of sequence reads of one or more nucleic acids, wherein the one or more nucleic acids are derived from a sample obtained from an individual; (b) analyze the plurality of sequence reads for the presence of a RET fusion nucleic acid molecule provided herein; and (c) detect, based on the analyzing, the RET fusion nucleic acid molecule in the sample. In some embodiments, a non-transitory computer readable storage medium of the disclosure comprises one or more programs executable by one or more computer processors for performing a method, comprising: (a) obtaining, using the one or more processors, a plurality of sequence reads of one or more nucleic acids, wherein the one or more nucleic acids are derived from a sample obtained from an individual; (b) analyzing, using the one or more processors, the plurality of sequence reads for the presence of a RET fusion nucleic acid molecule provided herein; and (c) detecting, using the one or more processors and based on the analyzing, the RET fusion nucleic acid molecule in the sample.

### A. RET Fusions

Certain aspects of the present disclosure relate to genomic rearrangements involving a rearranged during transfection (RET) gene, or a portion thereof. A RET rearrangement of the present disclosure may relate to any chromosomal translocation, fusion, or rearrangement involving the locus of a RET gene. In some embodiments, the rearrangements of the disclosure result in a RET fusion nucleic acid molecule that comprises at least a portion of a RET gene fused to at least a portion of another gene, such as any of CPEB3, OPTN, GRM7, AGBL4, PPP2R5A, OXR1, LRMDA, MGEA5, ADCY1, SAMD4A, HSD17B7P2, ACPP, ZNF248, MKX, MYH14, BMS1, GOLGB1, NTRK2, ABI3BP, ARHGAP19, GP2, SH2D3A, VSTM4, RBMS3, LINC00379, ZNF721, ZSWIM6, SGIP1, DCC, CCNY, WDFY4, EEA1, ELMO1, RAD1, NAALADL2, CCBE1, CEP135, RAI14, ADAMTS14, SORBS1, CSGALNACT2, PCM1, KIAA1217, MPRIP, TFG, SPECC1L, REEP3, RRBP1, ETV6, TAF3, RASGEF1A, TNIP2, SATB1, ALOX5, ANKRD26, CLIP1, DLG5, ERC1, FRMD4A, KIAA1468, NCOA4, PARD3, PRKAR1A, PRKG1, RUFY2, SNRNP70, SQSTM1, TNIP1, TRIM27, TRIM33, CCDC6, TRIM24, FGFR1OP, GAS2, HOOK1, LMNA, MLPH, MYH9, PCDH15, PIBF1, SLC12A2, ZNF485, ETV6, or any gene listed in Tables 1-2, below. Accordingly, certain aspects of the present disclosure relate to RET fusion nucleic acid molecules, as well as to RET fusion polypeptides encoded by such RET fusion nucleic acid molecules.

As used herein “rearranged during transfection” or “RET” refer to a gene encoding a RET mRNA or polypeptide. The RET gene encodes the RET receptor tyrosine kinase protein. RET is also known as RET proto-oncogene, PTC, MTC1, HSCR1, MEN2A, MEN2B, CDHF12, CDHR16, and RET-ELE1. In some embodiments, a RET gene is a human RET gene. An exemplary RET gene is represented by NCBI Gene ID No. 5979. Exemplary RET nucleotide sequences are represented by NCBI Ref. Seq. NM_020630 and NM_020975, and SEQ ID NOs: 133 and 134.

(SEQ ID NO: 133)
ATGGCGAAGGCGACGTCCGGTGCCGCGGGGCTGCGTCTGCTGTTGCTGCTGCTGCTGCCGCTGCTAGG
CAAAGTGGCATTGGGCCTCTACTTCTCGAGGGATGCTTACTGGGAGAAGCTGTATGTGGACCAGGCAG
CCGGCACGCCCTTGCTGTACGTCCATGCCCTGCGGGACGCCCCTGAGGAGGTGCCCAGCTTCCGCCTG
GGCCAGCATCTCTACGGCACGTACCGCACACGGCTGCATGAGAACAACTGGATCTGCATCCAGGAGGA
CACCGGCCTCCTCTACCTTAACCGGAGCCTGGACCATAGCTCCTGGGAGAAGCTCAGTGTCCGCAACC
GCGGCTTTCCCCTGCTCACCGTCTACCTCAAGGTCTTCCTGTCACCCACATCCCTTCGTGAGGGCGAG
TGCCAGTGGCCAGGCTGTGCCCGCGTATACTTCTCCTTCTTCAACACCTCCTTTCCAGCCTGCAGCTC
CCTCAAGCCCCGGGAGCTCTGCTTCCCAGAGACAAGGCCCTCCTTCCGCATTCGGGAGAACCGACCCC
CAGGCACCTTCCACCAGTTCCGCCTGCTGCCTGTGCAGTTCTTGTGCCCCAACATCAGCGTGGCCTAC
AGGCTCCTGGAGGGTGAGGGTCTGCCCTTCCGCTGCGCCCCGGACAGCCTGGAGGTGAGCACGCGCTG
GGCCCTGGACCGCGAGCAGCGGGAGAAGTACGAGCTGGTGGCCGTGTGCACCGTGCACGCCGGCGCGC
GCGAGGAGGTGGTGATGGTGCCCTTCCCGGTGACCGTGTACGACGAGGACGACTCGGCGCCCACCTTC
CCCGCGGGCGTCGACACCGCCAGCGCCGTGGTGGAGTTCAAGCGGAAGGAGGACACCGTGGTGGCCAC
GCTGCGTGTCTTCGATGCAGACGTGGTACCTGCATCAGGGGAGCTGGTGAGGCGGTACACAAGCACGC
TGCTCCCCGGGGACACCTGGGCCCAGCAGACCTTCCGGGTGGAACACTGGCCCAACGAGACCTCGGTC
CAGGCCAACGGCAGCTTCGTGCGGGCGACCGTACATGACTATAGGCTGGTTCTCAACCGGAACCTCTC
CATCTCGGAGAACCGCACCATGCAGCTGGCGGTGCTGGTCAATGACTCAGACTTCCAGGGCCCAGGAG
CGGGCGTCCTCTTGCTCCACTTCAACGTGTCGGTGCTGCCGGTCAGCCTGCACCTGCCCAGTACCTAC
TCCCTCTCCGTGAGCAGGAGGGCTCGCCGATTTGCCCAGATCGGGAAAGTCTGTGTGGAAAACTGCCA
GGCATTCAGTGGCATCAACGTCCAGTACAAGCTGCATTCCTCTGGTGCCAACTGCAGCACGCTAGGGG
TGGTCACCTCAGCCGAGGACACCTCGGGGATCCTGTTTGTGAATGACACCAAGGCCCTGCGGCGGCCC
AAGTGTGCCGAACTTCACTACATGGTGGTGGCCACCGACCAGCAGACCTCTAGGCAGGCCCAGGCCCA
GCTGCTTGTAACAGTGGAGGGGTCATATGTGGCCGAGGAGGCGGGCTGCCCCCTGTCCTGTGCAGTCA
GCAAGAGACGGCTGGAGTGTGAGGAGTGTGGCGGCCTGGGCTCCCCAACAGGCAGGTGTGAGTGGAGG
CAAGGAGATGGCAAAGGGATCACCAGGAACTTCTCCACCTGCTCTCCCAGCACCAAGACCTGCCCCGA
CGGCCACTGCGATGTTGTGGAGACCCAAGACATCAACATTTGCCCTCAGGACTGCCTCCGGGGCAGCA
TTGTTGGGGGACACGAGCCTGGGGAGCCCCGGGGGATTAAAGCTGGCTATGGCACCTGCAACTGCTTC
CCTGAGGAGGAGAAGTGCTTCTGCGAGCCCGAAGACATCCAGGATCCACTGTGCGACGAGCTGTGCCG
CACGGTGATCGCAGCCGCTGTCCTCTTCTCCTTCATCGTCTCGGTGCTGCTGTCTGCCTTCTGCATCC
ACTGCTACCACAAGTTTGCCCACAAGCCACCCATCTCCTCAGCTGAGATGACCTTCCGGAGGCCCGCC
CAGGCCTTCCCGGTCAGCTACTCCTCTTCCGGTGCCCGCCGGCCCTCGCTGGACTCCATGGAGAACCA
GGTCTCCGTGGATGCCTTCAAGATCCTGGAGGATCCAAAGTGGGAATTCCCTCGGAAGAACTTGGTTC
TTGGAAAAACTCTAGGAGAAGGCGAATTTGGAAAAGTGGTCAAGGCAACGGCCTTCCATCTGAAAGGC
AGAGCAGGGTACACCACGGTGGCCGTGAAGATGCTGAAAGAGAACGCCTCCCCGAGTGAGCTGCGAGA
CCTGCTGTCAGAGTTCAACGTCCTGAAGCAGGTCAACCACCCACATGTCATCAAATTGTATGGGGCCT
GCAGCCAGGATGGCCCGCTCCTCCTCATCGTGGAGTACGCCAAATACGGCTCCCTGCGGGGCTTCCTC
CGCGAGAGCCGCAAAGTGGGGCCTGGCTACCTGGGCAGTGGAGGCAGCCGCAACTCCAGCTCCCTGGA
CCACCCGGATGAGCGGGCCCTCACCATGGGCGACCTCATCTCATTTGCCTGGCAGATCTCACAGGGGA
TGCAGTATCTGGCCGAGATGAAGCTCGTTCATCGGGACTTGGCAGCCAGAAACATCCTGGTAGCTGAG
GGGCGGAAGATGAAGATTTCGGATTTCGGCTTGTCCCGAGATGTTTATGAAGAGGATTCCTACGTGAA
GAGGAGCCAGGGTCGGATTCCAGTTAAATGGATGGCAATTGAATCCCTTTTTGATCATATCTACACCA
CGCAAAGTGATGTATGGTCTTTTGGTGTCCTGCTGTGGGAGATCGTGACCCTAGGGGGAAACCCCTAT
CCTGGGATTCCTCCTGAGCGGCTCTTCAACCTTCTGAAGACCGGCCACCGGATGGAGAGGCCAGACAA
CTGCAGCGAGGAGATGTACCGCCTGATGCTGCAATGCTGGAAGCAGGAGCCGGACAAAAGGCCGGTGT
TTGCGGACATCAGCAAAGACCTGGAGAAGATGATGGTTAAGAGGAGAGACTACTTGGACCTTGCGGCG
TCCACTCCATCTGACTCCCTGATTTATGACGACGGCCTCTCAGAGGAGGAGACACCGCTGGTGGACTG
TAATAATGCCCCCCTCCCTCGAGCCCTCCCTTCCACATGGATTGAAAACAAACTCTATGGTAGAATTT
CCCATGCATTTACTAGATTCTAG
(SEQ ID NO: 134)
ATGGCGAAGGCGACGTCCGGTGCCGCGGGGCTGCGTCTGCTGTTGCTGCTGCTGCTGCCGCTGCTAGG
CAAAGTGGCATTGGGCCTCTACTTCTCGAGGGATGCTTACTGGGAGAAGCTGTATGTGGACCAGGCAG
CCGGCACGCCCTTGCTGTACGTCCATGCCCTGCGGGACGCCCCTGAGGAGGTGCCCAGCTTCCGCCTG
GGCCAGCATCTCTACGGCACGTACCGCACACGGCTGCATGAGAACAACTGGATCTGCATCCAGGAGGA
CACCGGCCTCCTCTACCTTAACCGGAGCCTGGACCATAGCTCCTGGGAGAAGCTCAGTGTCCGCAACC
GCGGCTTTCCCCTGCTCACCGTCTACCTCAAGGTCTTCCTGTCACCCACATCCCTTCGTGAGGGCGAG
TGCCAGTGGCCAGGCTGTGCCCGCGTATACTTCTCCTTCTTCAACACCTCCTTTCCAGCCTGCAGCTC
CCTCAAGCCCCGGGAGCTCTGCTTCCCAGAGACAAGGCCCTCCTTCCGCATTCGGGAGAACCGACCCC
CAGGCACCTTCCACCAGTTCCGCCTGCTGCCTGTGCAGTTCTTGTGCCCCAACATCAGCGTGGCCTAC
AGGCTCCTGGAGGGTGAGGGTCTGCCCTTCCGCTGCGCCCCGGACAGCCTGGAGGTGAGCACGCGCTG
GGCCCTGGACCGCGAGCAGCGGGAGAAGTACGAGCTGGTGGCCGTGTGCACCGTGCACGCCGGCGCGC
GCGAGGAGGTGGTGATGGTGCCCTTCCCGGTGACCGTGTACGACGAGGACGACTCGGCGCCCACCTTC
CCCGCGGGCGTCGACACCGCCAGCGCCGTGGTGGAGTTCAAGCGGAAGGAGGACACCGTGGTGGCCAC
GCTGCGTGTCTTCGATGCAGACGTGGTACCTGCATCAGGGGAGCTGGTGAGGCGGTACACAAGCACGC
TGCTCCCCGGGGACACCTGGGCCCAGCAGACCTTCCGGGTGGAACACTGGCCCAACGAGACCTCGGTC
CAGGCCAACGGCAGCTTCGTGCGGGCGACCGTACATGACTATAGGCTGGTTCTCAACCGGAACCTCTC
CATCTCGGAGAACCGCACCATGCAGCTGGCGGTGCTGGTCAATGACTCAGACTTCCAGGGCCCAGGAG
CGGGCGTCCTCTTGCTCCACTTCAACGTGTCGGTGCTGCCGGTCAGCCTGCACCTGCCCAGTACCTAC
TCCCTCTCCGTGAGCAGGAGGGCTCGCCGATTTGCCCAGATCGGGAAAGTCTGTGTGGAAAACTGCCA
GGCATTCAGTGGCATCAACGTCCAGTACAAGCTGCATTCCTCTGGTGCCAACTGCAGCACGCTAGGGG
TGGTCACCTCAGCCGAGGACACCTCGGGGATCCTGTTTGTGAATGACACCAAGGCCCTGCGGCGGCCC
AAGTGTGCCGAACTTCACTACATGGTGGTGGCCACCGACCAGCAGACCTCTAGGCAGGCCCAGGCCCA
GCTGCTTGTAACAGTGGAGGGGTCATATGTGGCCGAGGAGGCGGGCTGCCCCCTGTCCTGTGCAGTCA
GCAAGAGACGGCTGGAGTGTGAGGAGTGTGGCGGCCTGGGCTCCCCAACAGGCAGGTGTGAGTGGAGG
CAAGGAGATGGCAAAGGGATCACCAGGAACTTCTCCACCTGCTCTCCCAGCACCAAGACCTGCCCCGA
CGGCCACTGCGATGTTGTGGAGACCCAAGACATCAACATTTGCCCTCAGGACTGCCTCCGGGGCAGCA
TTGTTGGGGGACACGAGCCTGGGGAGCCCCGGGGGATTAAAGCTGGCTATGGCACCTGCAACTGCTTC
CCTGAGGAGGAGAAGTGCTTCTGCGAGCCCGAAGACATCCAGGATCCACTGTGCGACGAGCTGTGCCG
CACGGTGATCGCAGCCGCTGTCCTCTTCTCCTTCATCGTCTCGGTGCTGCTGTCTGCCTTCTGCATCC
ACTGCTACCACAAGTTTGCCCACAAGCCACCCATCTCCTCAGCTGAGATGACCTTCCGGAGGCCCGCC
CAGGCCTTCCCGGTCAGCTACTCCTCTTCCGGTGCCCGCCGGCCCTCGCTGGACTCCATGGAGAACCA
GGTCTCCGTGGATGCCTTCAAGATCCTGGAGGATCCAAAGTGGGAATTCCCTCGGAAGAACTTGGTTC
TTGGAAAAACTCTAGGAGAAGGCGAATTTGGAAAAGTGGTCAAGGCAACGGCCTTCCATCTGAAAGGC
AGAGCAGGGTACACCACGGTGGCCGTGAAGATGCTGAAAGAGAACGCCTCCCCGAGTGAGCTGCGAGA
CCTGCTGTCAGAGTTCAACGTCCTGAAGCAGGTCAACCACCCACATGTCATCAAATTGTATGGGGCCT
GCAGCCAGGATGGCCCGCTCCTCCTCATCGTGGAGTACGCCAAATACGGCTCCCTGCGGGGCTTCCTC
CGCGAGAGCCGCAAAGTGGGGCCTGGCTACCTGGGCAGTGGAGGCAGCCGCAACTCCAGCTCCCTGGA
CCACCCGGATGAGCGGGCCCTCACCATGGGCGACCTCATCTCATTTGCCTGGCAGATCTCACAGGGGA
TGCAGTATCTGGCCGAGATGAAGCTCGTTCATCGGGACTTGGCAGCCAGAAACATCCTGGTAGCTGAG
GGGCGGAAGATGAAGATTTCGGATTTCGGCTTGTCCCGAGATGTTTATGAAGAGGATTCCTACGTGAA
GAGGAGCCAGGGTCGGATTCCAGTTAAATGGATGGCAATTGAATCCCTTTTTGATCATATCTACACCA
CGCAAAGTGATGTATGGTCTTTTGGTGTCCTGCTGTGGGAGATCGTGACCCTAGGGGGAAACCCCTAT
CCTGGGATTCCTCCTGAGCGGCTCTTCAACCTTCTGAAGACCGGCCACCGGATGGAGAGGCCAGACAA
CTGCAGCGAGGAGATGTACCGCCTGATGCTGCAATGCTGGAAGCAGGAGCCGGACAAAAGGCCGGTGT
TTGCGGACATCAGCAAAGACCTGGAGAAGATGATGGTTAAGAGGAGAGACTACTTGGACCTTGCGGCG
TCCACTCCATCTGACTCCCTGATTTATGACGACGGCCTCTCAGAGGAGGAGACACCGCTGGTGGACTG
TAATAATGCCCCCCTCCCTCGAGCCCTCCCTTCCACATGGATTGAAAACAAACTCTATGGCATGTCAG
ACCCGAACTGGCCTGGAGAGAGTCCTGTACCACTCACGAGAGCTGATGGCACTAACACTGGGTTTCCA
AGATATCCAAATGATAGTGTATATGCTAACTGGATGCTTTCACCCTCAGCGGCAAAATTAATGGACAC
GTTTGATAGTTAA

Exemplary amino acid sequences of a RET polypeptide are represented by NCBI Ref. Seq. NP_065681 and NP_066124, and SEQ ID NOs: 135 and 136.

(SEQ ID NO: 135)
MAKATSGAAGLRLLLLLLLPLLGKVALGLYFSRDAYWEKLYVDQAAGTPLLYVHALRDAPEEVPSFRL
GQHLYGTYRTRLHENNWICIQEDTGLLYLNRSLDHSSWEKLSVRNRGFPLLTVYLKVFLSPTSLREGE
CQWPGCARVYFSFFNTSFPACSSLKPRELCFPETRPSFRIRENRPPGTFHQFRLLPVQFLCPNISVAY
RLLEGEGLPFRCAPDSLEVSTRWALDREQREKYELVAVCTVHAGAREEVVMVPFPVTVYDEDDSAPTF
PAGVDTASAVVEFKRKEDTVVATLRVFDADVVPASGELVRRYTSTLLPGDTWAQQTFRVEHWPNETSV
QANGSFVRATVHDYRLVLNRNLSISENRTMQLAVLVNDSDFQGPGAGVLLLHFNVSVLPVSLHLPSTY
SLSVSRRARRFAQIGKVCVENCQAFSGINVQYKLHSSGANCSTLGVVTSAEDTSGILFVNDTKALRRP
KCAELHYMVVATDQQTSRQAQAQLLVTVEGSYVAEEAGCPLSCAVSKRRLECEECGGLGSPTGRCEWR
QGDGKGITRNFSTCSPSTKTCPDGHCDVVETQDINICPQDCLRGSIVGGHEPGEPRGIKAGYGTCNCF
PEEEKCFCEPEDIQDPLCDELCRTVIAAAVLFSFIVSVLLSAFCIHCYHKFAHKPPISSAEMTFRRPA
QAFPVSYSSSGARRPSLDSMENQVSVDAFKILEDPKWEFPRKNLVLGKTLGEGEFGKVVKATAFHLKG
RAGYTTVAVKMLKENASPSELRDLLSEFNVLKQVNHPHVIKLYGACSQDGPLLLIVEYAKYGSLRGFL
RESRKVGPGYLGSGGSRNSSSLDHPDERALTMGDLISFAWQISQGMQYLAEMKLVHRDLAARNILVAE
GRKMKISDFGLSRDVYEEDSYVKRSQGRIPVKWMAIESLFDHIYTTQSDVWSFGVLLWEIVTLGGNPY
PGIPPERLFNLLKTGHRMERPDNCSEEMYRLMLQCWKQEPDKRPVFADISKDLEKMMVKRRDYLDLAA
STPSDSLIYDDGLSEEETPLVDCNNAPLPRALPSTWIENKLYGRISHAFTRF
(SEQ ID NO: 136)
MAKATSGAAGLRLLLLLLLPLLGKVALGLYFSRDAYWEKLYVDQAAGTPLLYVHALRDAPEEVPSFRL
GQHLYGTYRTRLHENNWICIQEDTGLLYLNRSLDHSSWEKLSVRNRGFPLLTVYLKVFLSPTSLREGE
CQWPGCARVYFSFFNTSFPACSSLKPRELCFPETRPSFRIRENRPPGTFHQFRLLPVQFLCPNISVAY
RLLEGEGLPERCAPDSLEVSTRWALDREQREKYELVAVCTVHAGAREEVVMVPFPVTVYDEDDSAPTF
PAGVDTASAVVEFKRKEDTVVATLRVFDADVVPASGELVRRYTSTLLPGDTWAQQTERVEHWPNETSV
QANGSFVRATVHDYRLVLNRNLSISENRTMQLAVLVNDSDFQGPGAGVLLLHFNVSVLPVSLHLPSTY
SLSVSRRARRFAQIGKVCVENCQAFSGINVQYKLHSSGANCSTLGVVTSAEDTSGILFVNDTKALRRP
KCAELHYMVVATDQQTSRQAQAQLLVTVEGSYVAEEAGCPLSCAVSKRRLECEECGGLGSPTGRCEWR
QGDGKGITRNFSTCSPSTKTCPDGHCDVVETQDINICPQDCLRGSIVGGHEPGEPRGIKAGYGTCNCF
PEEEKCFCEPEDIQDPLCDELCRTVIAAAVLFSFIVSVLLSAFCIHCYHKFAHKPPISSAEMTFRRPA
QAFPVSYSSSGARRPSLDSMENQVSVDAFKILEDPKWEFPRKNLVLGKTLGEGEFGKVVKATAFHLKG
RAGYTTVAVKMLKENASPSELRDLLSEFNVLKQVNHPHVIKLYGACSQDGPLLLIVEYAKYGSLRGFL
RESRKVGPGYLGSGGSRNSSSLDHPDERALTMGDLISFAWQISQGMQYLAEMKLVHRDLAARNILVAE
GRKMKISDFGLSRDVYEEDSYVKRSQGRIPVKWMAIESLFDHIYTTQSDVWSFGVLLWEIVTLGGNPY
PGIPPERLFNLLKTGHRMERPDNCSEEMYRLMLQCWKQEPDKRPVFADISKDLEKMMVKRRDYLDLAA
STPSDSLIYDDGLSEEETPLVDCNNAPLPRALPSTWIENKLYGMSDPNWPGESPVPLTRADGTNTGFP
RYPNDSVYANWMLSPSAAKLMDTFDS

An exemplary transcript sequence of a GOLGB1 gene is represented by NCBI Ref. Seq. NM_004487. An exemplary transcript sequence of a GP2 gene is represented by NCBI Ref. Seq. NM_001502. An exemplary transcript sequence of a PPP2R5A gene is represented by NCBI Ref. Seq. NM_006243. An exemplary transcript sequence of a GRM7 gene is represented by NCBI Ref. Seq. NM_000844. An exemplary transcript sequence of a MYH14 gene is represented by NCBI Ref. Seq. NM_024729. An exemplary transcript sequence of an MGEA5 gene is represented by NCBI Ref. Seq. NM_012215. An exemplary transcript sequence of a SAMD4A gene is represented by NCBI Ref. Seq. NM_015589. An exemplary transcript sequence of an ACPP gene is represented by NCBI Ref. Seq. NM_001134194. An exemplary transcript sequence of a BMS1 gene is represented by NCBI Ref. Seq. NM_014753. An exemplary transcript sequence of a CEP135 gene is represented by NCBI Ref. Seq. NM_025009. An exemplary transcript sequence of an EEA1 gene is represented by NCBI Ref. Seq. NM_003566. An exemplary transcript sequence of a CSGALNACT2 gene is represented by NCBI Ref. Seq. NM_018590. An exemplary transcript sequence of a KIAA1217 gene is represented by NCBI Ref. Seq. NM_019590. An exemplary transcript sequence of a SORBS1 gene is represented by NCBI Ref. Seq. NM_006434. An exemplary transcript sequence of an MPRIP gene is represented by NCBI Ref. Seq. NM_015134. An exemplary transcript sequence of a TFG gene is represented by NCBI Ref. Seq. NM_006070. An exemplary transcript sequence of a SPECC1L gene is represented by NCBI Ref. Seq. NM_015330. An exemplary transcript sequence of a REEP3 gene is represented by NCBI Ref. Seq. NM_001001330. An exemplary transcript sequence of a RRBP1 gene is represented by NCBI Ref. Seq. NM_004587. An exemplary transcript sequence of an ETV6 gene is represented by NCBI Ref. Seq. NM_001987. An exemplary transcript sequence of a TAF3 gene is represented by NCBI Ref. Seq. NM_031923. An exemplary transcript sequence of a PCM1 gene is represented by NCBI Ref. Seq. NM_006197. An exemplary transcript sequence of a TNIP2 gene is represented by NCBI Ref. Seq. NM_024309. An exemplary transcript sequence of a SATB1 gene is represented by NCBI Ref. Seq. NM_002971. An exemplary transcript sequence of an ADCY1 gene is represented by NCBI Ref. Seq. NM_021116. An exemplary transcript sequence of a ZNF248 gene is represented by NCBI Ref. Seq. NM_021045. An exemplary transcript sequence of an AGBL4 gene is represented by NCBI Ref. Seq. NM_032785. An exemplary transcript sequence of an LRMDA gene is represented by NCBI Ref. Seq. NM_032024. An exemplary transcript sequence of an ARHGAP19 gene is represented by NCBI Ref. Seq. NM_032900. An exemplary transcript sequence of a CPEB3 gene is represented by NCBI Ref. Seq. NM_014912. An exemplary transcript sequence of a DCC gene is represented by NCBI Ref. Seq. NM_005215. An exemplary transcript sequence of an ELMO1 gene is represented by NCBI Ref. Seq. NM_014800. An exemplary transcript sequence of a WDFY4 gene is represented by NCBI Ref. Seq. NM_020945. An exemplary transcript sequence of a MKX gene is represented by NCBI Ref. Seq. NM_173576. An exemplary transcript sequence of a RBMS3 gene is represented by NCBI Ref. Seq. NM_014483. An exemplary transcript sequence of a SGIP1 gene is represented by NCBI Ref. Seq. NM_032291. An exemplary transcript sequence of a ZSWIM6 gene is represented by NCBI Ref. Seq. NM_020928. An exemplary transcript sequence of an ALOX5 gene is represented by NCBI Ref. Seq. NM_000698. An exemplary transcript sequence of a NTRK2 gene is represented by NCBI Ref. Seq. NM_006180. An exemplary transcript sequence of a OXR1 gene is represented by NCBI Ref. Seq. NM_018002. An exemplary transcript sequence of a CCBE1 gene is represented by NCBI Ref. Seq. NM_133459. An exemplary transcript sequence of a NAALADL2 gene is represented by NCBI Ref. Seq. NM_207015. An exemplary transcript sequence of a RAI14 gene is represented by NCBI Ref. Seq. NM_015577. An exemplary transcript sequence of a ABI3BP gene is represented by NCBI Ref. Seq. NM_015429. An exemplary transcript sequence of a LINC00379 gene is represented by NCBI Ref. Seq. NR_047004. An exemplary transcript sequence of a OPTN gene is represented by NCBI Ref. Seq. NM_021980. An exemplary transcript sequence of a SH2D3A gene is represented by NCBI Ref. Seq. NM_005490. An exemplary transcript sequence of a ZNF721 gene is represented by NCBI Ref. Seq. NM_133474. An exemplary transcript sequence of a ADAMTS14 gene is represented by NCBI Ref. Seq. NM_080722. An exemplary transcript sequence of a CCNY gene is represented by NCBI Ref. Seq. NM_181698. An exemplary transcript sequence of a RASGEF1A gene is represented by NCBI Ref. Seq. NM_145313. An exemplary transcript sequence of a HSD17B7P2 gene is represented by NCBI Ref. Seq. NR_003086. An exemplary transcript sequence of a RAD1 gene is represented by NCBI Ref. Seq. NM_002853. An exemplary transcript sequence of a VSTM4 gene is represented by NCBI Ref. Seq. NM_001031746. An exemplary transcript sequence of an ANKRD26 gene is represented by NCBI Ref. Seq. NM_014915. An exemplary transcript sequence of a CLIP1 gene is represented by NCBI Ref. Seq. NM_002956. An exemplary transcript sequence of a DLG5 gene is represented by NCBI Ref. Seq. NM_004747. An exemplary transcript sequence of an ERC1 gene is represented by NCBI Ref. Seq. NM_178039. An exemplary transcript sequence of a FRMD4A gene is represented by NCBI Ref. Seq. NM_018027. An exemplary transcript sequence of a KIAA1468 gene is represented by NCBI Ref. Seq. NM_020854. An exemplary transcript sequence of a NCOA4 gene is represented by NCBI Ref. Seq. NM_005437. An exemplary transcript sequence of a PARD3 gene is represented by NCBI Ref. Seq. NM_019619. An exemplary transcript sequence of a PRKAR1A gene is represented by NCBI Ref. Seq. NM_002734. An exemplary transcript sequence of a PRKG1 gene is represented by NCBI Ref. Seq. NM_006258. An exemplary transcript sequence of a RUFY2 gene is represented by NCBI Ref. Seq. NM_017987. An exemplary transcript sequence of a SNRNP70 gene is represented by NCBI Ref. Seq. NM_003089. An exemplary transcript sequence of a SQSTM1 gene is represented by NCBI Ref. Seq. NM_003900. An exemplary transcript sequence of a TNIP1 gene is represented by NCBI Ref. Seq. NM_006058. An exemplary transcript sequence of a TRIM27 gene is represented by NCBI Ref. Seq. NM_006510. An exemplary transcript sequence of a TRIM33 gene is represented by NCBI Ref. Seq. NM_015906. An exemplary transcript sequence of a CCDC6 gene is represented by NCBI Ref. Seq. NM_005436. An exemplary transcript sequence of a TRIM24 gene is represented by NCBI Ref. Seq. NM_003852. An exemplary transcript sequence of a FGFR1OP gene is represented by NCBI Ref. Seq. NM_007045. An exemplary transcript sequence of a GAS2 gene is represented by NCBI Ref. Seq. NM_005256. An exemplary transcript sequence of a HOOK1 gene is represented by NCBI Ref. Seq. NM_015888. An exemplary transcript sequence of a LMNA gene is represented by NCBI Ref. Seq. NM_005572. An exemplary transcript sequence of a MLPH gene is represented by NCBI Ref. Seq. NM_024101. An exemplary transcript sequence of a MYH9 gene is represented by NCBI Ref. Seq. NM_002473. An exemplary transcript sequence of a PCDH15 gene is represented by NCBI Ref. Seq. NM_033056. An exemplary transcript sequence of a PIBF1 gene is represented by NCBI Ref. Seq. NM_006346. An exemplary transcript sequence of a SLC12A2 gene is represented by NCBI Ref. Seq. NM_001046. An exemplary transcript sequence of a ZNF485 gene is represented by NCBI Ref. Seq. NM_145312.

### (i) Exemplary RET Fusion Nucleic Acid Molecules

In some aspects, provided herein are RET fusion nucleic acid molecules comprising at least a portion of a RET gene fused to at least a portion of another gene.

In some embodiments, a RET fusion nucleic acid molecule of the disclosure comprises at least a portion of a RET gene and at least a portion of a CPEB3, OPTN, GRM7, AGBL4, PPP2R5A, OXR1, LRMDA, MGEA5, ADCY1, SAMD4A, HSD17B7P2, ACPP, ZNF248, MKX, MYH14, BMS1, GOLGB1, NTRK2, ABI3BP, ARHGAP19, GP2, SH2D3A, VSTM4, RBMS3, LINC00379, ZNF721, ZSWIM6, SGIP1, DCC, CCNY, WDFY4, EEA1, ELMO1, RAD1, NAALADL2, CCBE1, CEP135, RAI14, ADAMTS14, SORBS1, CSGALNACT2, PCM1, KIAA1217, MPRIP, TFG, SPECC1L, REEP3, RRBP1, ETV6, TAF3, RASGEF1A, TNIP2, SATB1, ALOX5, ANKRD26, CLIP1, DLG5, ERC1, FRMD4A, KIAA1468, NCOA4, PARD3, PRKAR1A, PRKG1, RUFY2, SNRNP70, SQSTM1, TNIP1, TRIM27, TRIM33, CCDC6, TRIM24, FGFR1OP, GAS2, HOOK1, LMNA, MLPH, MYH9, PCDH15, PIBF1, SLC12A2, ZNF485, or ETV6 gene, or of any gene listed in Tables 1-2, below.

TABLE 1
RET gene fusion partners.

| | | | |
|---|---|---|---|
| CPEB3 | OPTN | GRM7 | AGBL4 |
| PPP2R5A | OXR1 | LRMDA | MGEA5 |
| ADCY1 | SAMD4A | HSD17B7P2 | ACPP |
| ZNF248 | MKX | MYH14 | BMS1 |
| GOLGB1 | NTRK2 | ABI3BP | ARHGAP19 |
| GP2 | SH2D3A | VSTM4 | RBMS3 |
| LINC00379 | ZNF721 | ZSWIM6 | SGIP1 |
| DCC | CCNY | WDFY4 | EEA1 |
| ELMO1 | RAD1 | NAALADL2 | CCBE1 |
| CEP135 | RAI14 | ADAMTS14 | FGFR1OP |
| GAS2 | HOOK1 | LMNA | MLPH |
| MYH9 | PCDH15 | PIBF1 | SLC12A2 |
| ZNF485 | ETV6 | | |

TABLE 2
RET gene fusion partners and corresponding cancer types.

| RET Fusion Partner | |
|---|---|
| Gene | Cancer Type |
| SORBS1 | Soft tissue leiomyosarcoma |
| CSGALNACT2 | Uterus carcinosarcoma |
| CSGALNACT2 | Bladder urothelial (transitional cell) |
| | carcinoma |
| PCM1 | Brain glioblastoma (GBM) |
| PCM1 | Pancreas ductal adenocarcinoma |
| KIAA1217 | Pancreas ductal adenocarcinoma |
| MPRIP | Soft tissue sarcoma (NOS) |
| TFG | Pancreas ductal adenocarcinoma |
| SPECC1L | Pancreas ductal adenocarcinoma |
| REEP3 | Esophagus adenocarcinoma |
| RRBP1 | Ovary serous carcinoma |
| ETV6 | Pancreas ductal adenocarcinoma |
| TAF3 | Colon neuroendocrine carcinoma |
| RASGEF1A | Unknown primary melanoma |
| TNIP2 | Colon adenocarcinoma (CRC) |
| SATB1 | Pancreas ductal adenocarcinoma |
| ALOX5 | Brain glioblastoma (GBM) |
| ANKRD26 | Lung adenocarcinoma |
| CLIP1 | Lung adenocarcinoma |
| DLG5 | Ovary serous carcinoma |
| DLG5 | Breast carcinoma |
| ERC1 | Soft tissue sarcoma |
| ERC1 | Unknown primary carcinoma |
| ERC1 | Pancreatobiliary carcinoma |
| FRMD4A | Unknown primary adenocarcinoma |
| KIAA1468 | Thyroid papillary carcinoma |
| NCOA4 | Colon adenocarcinoma (CRC) |
| PARD3 | Unknown primary urothelial carcinoma |
| PARD3 | Unknown primary neuroendocrine tumor |
| PRKARIA | Brain astrocytoma |
| PRKG1 | Prostate acinar adenocarcinoma |
| PRKG1 | Lung adenocarcinoma |
| RUFY2 | Lung adenocarcinoma |
| SNRNP70 | Lung adenocarcinoma |
| SQSTM1 | Lung adenocarcinoma |
| TNIP1 | Lung adenocarcinoma |
| TRIM27 | Colon adenocarcinoma (CRC) |
| TRIM33 | Pancreas ductal adenocarcinoma |
| CCDC6 | Ovary epithelial carcinoma |
| TRIM24 | Lung adenocarcinoma |
| ETV6 | Salivary gland mammary analogue |
| | secretory carcinoma |

For example, in some embodiments, the RET fusion nucleic acid molecule is selected from a GOLGB1-RET, GP2-RET, PPP2R5A-RET, GRM7-RET, MYH14-RET, MGEA5-RET, SAMD4A-RET, ACPP-RET, BMS1-RET, CEP135-RET, EEA1-RET, CSGALNACT2-RET, KIAA1217-RET, SORBS1-RET, MPRIP-RET, TFG-RET, SPECC1L-RET, REEP3-RET, RRBP1-RET, ETV6-RET, TAF3-RET, PCM1-RET, TNIP2-RET, SATB1-RET, RET-ADCY1, RET-ZNF248, RET-AGBL4, RET-LRMDA, RET-ARHGAP19, RET-CPEB3, RET-DCC, RET-ELMO1, RET-WDFY4, MKX-RET, RBMS3-RET, SGIP1-RET, ZSWIM6-RET, ALOX5-RET, RET-NTRK2, RET-OXR1, RET-CCBE1, RET-NAALADL2, RAI14-RET, ABI3BP-RET, LINC00379-RET, OPTN-RET, SH2D3A-RET, ZNF721-RET, ADAMTS14-RET, CSGALNACT2-RET, CPEB3-RET, RET-CCNY, RET-RASGEF1A, RET-HSD17B7P2, RET-RAD1, RET-VSTM4, ANKRD26-RET, CLIP1-RET, DLG5-RET, ERC1-RET, FRMD4A-RET, KIAA1468-RET, NCOA4-RET, PARD3-RET, PRKAR1A-RET, PRKG1-RET, RUFY2-RET, SNRNP70-RET, SQSTM1-RET, TNIP1-RET, TRIM27-RET, TRIM33-RET, CCDC6-RET, TRIM24-RET, FGFR1OP-RET, GAS2-RET, HOOK1-RET, LMNA-RET, MLPH-RET, MYH9-RET, PCDH15-RET, PIBF1-RET, SLC12A2-RET, or ZNF485-RET fusion nucleic acid molecule.

In some embodiments, the RET fusion nucleic acid molecule of the disclosure comprises at least a portion of a RET gene and at least a portion of a CPEB3, OPTN, GRM7, AGBL4, PPP2R5A, OXR1, LRMDA, MGEA5, ADCY1, SAMD4A, HSD17B7P2, ACPP, ZNF248, MKX, MYH14, BMS1, GOLGB1, NTRK2, ABI3BP, ARHGAP19, GP2, SH2D3A, VSTM4, RBMS3, LINC00379, ZNF721, ZSWIM6, SGIP1, DCC, CCNY, WDFY4, EEA1, ELMO1, RAD1, NAALADL2, CCBE1, CEP135, RAI14, ADAMTS14, SORBS1, CSGALNACT2, PCM1, KIAA1217, MPRIP, TFG, SPECC1L, REEP3, RRBP1, ETV6, TAF3, RASGEF1A, TNIP2, SATB1, ALOX5, ANKRD26, CLIP1, DLG5, ERC1, FRMD4A, KIAA1468, NCOA4, PARD3, PRKAR1A, PRKG1, RUFY2, SNRNP70, SQSTM1, TNIP1, TRIM27, TRIM33, CCDC6, TRIM24, FGFR1OP, GAS2, HOOK1, LMNA, MLPH, MYH9, PCDH15, PIBF1, SLC12A2, or ZNF485 gene, or of any gene listed in Tables 1-2, wherein the order of the genes in the fusion in 5′ to 3′ direction is as indicated in Table 3.

TABLE 3
Order of fused genes in exemplary
RET fusion nucleic acid molecules.

| RET Fusion Nucleic Acid Molecule | 5′ Gene | 3′ Gene |
|---|---|---|
| GOLGB1 - RET | GOLGB1 | RET |
| GP2 - RET | GP2 | RET |
| PPP2R5A - RET | PPP2R5A | RET |
| GRM7 - RET | GRM7 | RET |
| MYH14 - RET | MYH14 | RET |
| MGEA5 - RET | MGEA5 | RET |
| SAMD4A - RET | SAMD4A | RET |
| ACPP - RET | ACPP | RET |
| BMS1 - RET | BMS1 | RET |
| CEP135 - RET | CEP135 | RET |
| EEA1 - RET | EEA1 | RET |
| CSGALNACT2 - RET | CSGALNACT2 | RET |
| KIAA1217 - RET | KIAA1217 | RET |
| SORBS1 - RET | SORBS1 | RET |
| MPRIP - RET | MPRIP | RET |
| TFG - RET | TFG | RET |
| SPECC1L - RET | SPECC1L | RET |
| REEP3 - RET | REEP3 | RET |
| RRBP1 - RET | RRBP1 | RET |
| ETV6 - RET | ETV6 | RET |
| TAF3 - RET | TAF3 | RET |
| PCM1 - RET | PCM1 | RET |
| TNIP2 - RET | TNIP2 | RET |
| SATB1 - RET | SATB1 | RET |
| RET - ADCY1 | RET | ADCY1 |
| RET - ZNF248 | RET | ZNF248 |
| RET - AGBL4 | RET | AGBL4 |
| RET - LRMDA | RET | LRMDA |
| RET - ARHGAP19 | RET | ARHGAP19 |
| RET - CPEB3 | RET | CPEB3 |
| RET - DCC | RET | DCC |
| RET - ELMO1 | RET | ELMO1 |
| RET - WDFY4 | RET | WDFY4 |
| MKX - RET | MKX | RET |
| RBMS3 - RET | RBMS3 | RET |
| SGIP1 - RET | SGIP1 | RET |
| ZSWIM6 - RET | ZSWIM6 | RET |
| ALOX5 - RET | ALOX5 | RET |
| RET - NTRK2 | RET | NTRK2 |
| RET - OXR1 | RET | OXR1 |
| RET - CCBE1 | RET | CCBE1 |
| RET - NAALADL2 | RET | NAALADL2 |
| RAI14 - RET | RAI14 | RET |
| ABI3BP - RET | ABI3BP | RET |
| LINC00379 - RET | LINC00379 | RET |
| OPTN - RET | OPTN | RET |
| SH2D3A - RET | SH2D3A | RET |
| ZNF721 - RET | ZNF721 | RET |
| ADAMTS14 - RET | ADAMTS14 | RET |
| CSGALNACT2 - RET | CSGALNACT2 | RET |
| CPEB3 - RET | CPEB3 | RET |
| RET - CCNY | RET | CCNY |
| RET - RASGEF1A | RET | RASGEF1A |
| RET - HSD17B7P2 | RET | HSD17B7P2 |
| RET - RAD1 | RET | RAD1 |
| RET - VSTM4 | RET | VSTM4 |
| ANKRD26 - RET | ANKRD26 | RET |
| CLIP1 - RET | CLIP1 | RET |
| DLG5 - RET | DLG5 | RET |
| ERC1 - RET | ERC1 | RET |
| FRMD4A - RET | FRMD4A | RET |
| KIAA1468 - RET | KIAA1468 | RET |
| NCOA4 - RET | NCOA4 | RET |
| PARD3 - RET | PARD3 | RET |
| PRKAR1A - RET | PRKAR1A | RET |
| PRKG1 - RET | PRKG1 | RET |
| RUFY2 - RET | RUFY2 | RET |
| SNRNP70 - RET | SNRNP70 | RET |
| SQSTM1 - RET | SQSTM1 | RET |
| TNIP1 - RET | TNIP1 | RET |
| TRIM27 - RET | TRIM27 | RET |
| TRIM33 - RET | TRIM33 | RET |
| CCDC6 - RET | CCDC6 | RET |
| TRIM24 - RET | TRIM24 | RET |
| FGFR1OP-RET | FGFR1OP | RET |
| GAS2-RET | GAS2 | RET |
| HOOK1-RET | HOOK1 | RET |
| LMNA-RET | LMNA | RET |
| MLPH-RET | MLPH | RET |
| MYH9-RET | MYH9 | RET |
| PCDH15-RET | PCDH15 | RET |
| PIBF1-RET | PIBF1 | RET |
| SLC12A2-RET | SLC12A2 | RET |
| ZNF485-RET | ZNF485 | RET |

In some embodiments, the GOLGB1-RET fusion nucleic acid molecule of the disclosure comprises, in 5′ to 3′ direction, a GOLGB1 gene or a portion thereof fused to a RET gene or a portion thereof. In some embodiments, the GP2-RET fusion nucleic acid molecule of the disclosure comprises, in 5′ to 3′ direction, a GP2 gene or a portion thereof fused to a RET gene or a portion thereof. In some embodiments, the PPP2RSA-RET fusion nucleic acid molecule of the disclosure comprises, in 5′ to 3′ direction, a PPP2R5A gene or a portion thereof fused to a RET gene or a portion thereof. In some embodiments, the GRM7-RET fusion nucleic acid molecule of the disclosure comprises, in 5′ to 3′ direction, a GRM7 gene or a portion thereof fused to a RET gene or a portion thereof. In some embodiments, the MYH14-RET fusion nucleic acid molecule of the disclosure comprises, in 5′ to 3′ direction, a MYH14 gene or a portion thereof fused to a RET gene or a portion thereof. In some embodiments, the MGEA5-RET fusion nucleic acid molecule of the disclosure comprises, in 5′ to 3′ direction, a MGEA5 gene or a portion thereof fused to a RET gene or a portion thereof. In some embodiments, the SAMD4A-RET fusion nucleic acid molecule of the disclosure comprises, in 5′ to 3′ direction, a SAMD4A gene or a portion thereof fused to a RET gene or a portion thereof. In some embodiments, the ACPP-RET fusion nucleic acid molecule of the disclosure comprises, in 5′ to 3′ direction, a ACPP gene or a portion thereof fused to a RET gene or a portion thereof. In some embodiments, the BMS1-RET fusion nucleic acid molecule of the disclosure comprises, in 5′ to 3′ direction, a BMS1 gene or a portion thereof fused to a RET gene or a portion thereof. In some embodiments, the CEP135-RET fusion nucleic acid molecule of the disclosure comprises, in 5′ to 3′ direction, a CEP135 gene or a portion thereof fused to a RET gene or a portion thereof. In some embodiments, the EEA1-RET fusion nucleic acid molecule of the disclosure comprises, in 5′ to 3′ direction, a EEA1 gene or a portion thereof fused to a RET gene or a portion thereof. In some embodiments, the CSGALNACT2-RET fusion nucleic acid molecule of the disclosure comprises, in 5′ to 3′ direction, a CSGALNACT2 gene or a portion thereof fused to a RET gene or a portion thereof. In some embodiments, the KIAA1217-RET fusion nucleic acid molecule of the disclosure comprises, in 5′ to 3′ direction, a KIAA1217 gene or a portion thereof fused to a RET gene or a portion thereof. In some embodiments, the SORBS1-RET fusion nucleic acid molecule of the disclosure comprises, in 5′ to 3′ direction, a SORBS1 gene or a portion thereof fused to a RET gene or a portion thereof. In some embodiments, the MPRIP-RET fusion nucleic acid molecule of the disclosure comprises, in 5′ to 3′ direction, a MPRIP gene or a portion thereof fused to a RET gene or a portion thereof. In some embodiments, the TFG-RET fusion nucleic acid molecule of the disclosure comprises, in 5′ to 3′ direction, a TFG gene or a portion thereof fused to a RET gene or a portion thereof. In some embodiments, the SPECC1L-RET fusion nucleic acid molecule of the disclosure comprises, in 5′ to 3′ direction, a SPECC1L gene or a portion thereof fused to a RET gene or a portion thereof. In some embodiments, the REEP3-RET fusion nucleic acid molecule of the disclosure comprises, in 5′ to 3′ direction, a REEP3 gene or a portion thereof fused to a RET gene or a portion thereof. In some embodiments, the RRBP1-RET fusion nucleic acid molecule of the disclosure comprises, in 5′ to 3′ direction, a RRBP1 gene or a portion thereof fused to a RET gene or a portion thereof. In some embodiments, the ETV6-RET fusion nucleic acid molecule of the disclosure comprises, in 5′ to 3′ direction, a ETV6 gene or a portion thereof fused to a RET gene or a portion thereof. In some embodiments, the TAF3-RET fusion nucleic acid molecule of the disclosure comprises, in 5′ to 3′ direction, a TAF3 gene or a portion thereof fused to a RET gene or a portion thereof. In some embodiments, the PCM1-RET fusion nucleic acid molecule of the disclosure comprises, in 5′ to 3′ direction, a PCM1 gene or a portion thereof fused to a RET gene or a portion thereof. In some embodiments, the TNIP2-RET fusion nucleic acid molecule of the disclosure comprises, in 5′ to 3′ direction, a TNIP2 gene or a portion thereof fused to a RET gene or a portion thereof. In some embodiments, the SATB1-RET fusion nucleic acid molecule of the disclosure comprises, in 5′ to 3′ direction, a SATB1 gene or a portion thereof fused to a RET gene or a portion thereof. In some embodiments, the RET-ADCY1 fusion nucleic acid molecule of the disclosure comprises, in 5′ to 3′ direction, a RET gene or a portion thereof fused to a ADCY1 gene or a portion thereof. In some embodiments, the RET-ZNF248 fusion nucleic acid molecule of the disclosure comprises, in 5′ to 3′ direction, a RET gene or a portion thereof fused to a ZNF248 gene or a portion thereof. In some embodiments, the RET-AGBL4 fusion nucleic acid molecule of the disclosure comprises, in 5′ to 3′ direction, a RET gene or a portion thereof fused to a AGBL4 gene or a portion thereof. In some embodiments, the RET-LRMDA fusion nucleic acid molecule of the disclosure comprises, in 5′ to 3′ direction, a RET gene or a portion thereof fused to a LRMDA gene or a portion thereof. In some embodiments, the RET-ARHGAP19 fusion nucleic acid molecule of the disclosure comprises, in 5′ to 3′ direction, a RET gene or a portion thereof fused to a ARHGAP19 gene or a portion thereof. In some embodiments, the RET-CPEB3 fusion nucleic acid molecule of the disclosure comprises, in 5′ to 3′ direction, a RET gene or a portion thereof fused to a CPEB3 gene or a portion thereof. In some embodiments, the RET-DCC fusion nucleic acid molecule of the disclosure comprises, in 5′ to 3′ direction, a RET gene or a portion thereof fused to a DCC gene or a portion thereof. In some embodiments, the RET-ELMO1 fusion nucleic acid molecule of the disclosure comprises, in 5′ to 3′ direction, a RET gene or a portion thereof fused to a ELMO1 gene or a portion thereof. In some embodiments, the RET-WDFY4 fusion nucleic acid molecule of the disclosure comprises, in 5′ to 3′ direction, a RET gene or a portion thereof fused to a WDFY4 gene or a portion thereof. In some embodiments, the MKX-RET fusion nucleic acid molecule of the disclosure comprises, in 5′ to 3′ direction, a MKX gene or a portion thereof fused to a RET gene or a portion thereof. In some embodiments, the RBMS3-RET fusion nucleic acid molecule of the disclosure comprises, in 5′ to 3′ direction, a RBMS3 gene or a portion thereof fused to a RET gene or a portion thereof. In some embodiments, the SGIP1-RET fusion nucleic acid molecule of the disclosure comprises, in 5′ to 3′ direction, a SGIP1 gene or a portion thereof fused to a RET gene or a portion thereof. In some embodiments, the ZSWIM6-RET fusion nucleic acid molecule of the disclosure comprises, in 5′ to 3′ direction, a ZSWIM6 gene or a portion thereof fused to a RET gene or a portion thereof. In some embodiments, the ALOX5-RET fusion nucleic acid molecule of the disclosure comprises, in 5′ to 3′ direction, a ALOX5 gene or a portion thereof fused to a RET gene or a portion thereof. In some embodiments, the RET-NTRK2 fusion nucleic acid molecule of the disclosure comprises, in 5′ to 3′ direction, a RET gene or a portion thereof fused to a NTRK2 gene or a portion thereof. In some embodiments, the RET-OXR1 fusion nucleic acid molecule of the disclosure comprises, in 5′ to 3′ direction, a RET gene or a portion thereof fused to a OXR1 gene or a portion thereof. In some embodiments, the RET-CCBE1 fusion nucleic acid molecule of the disclosure comprises, in 5′ to 3′ direction, a RET gene or a portion thereof fused to a CCBE1 gene or a portion thereof. In some embodiments, the RET-NAALADL2 fusion nucleic acid molecule of the disclosure comprises, in 5′ to 3′ direction, a RET gene or a portion thereof fused to a NAALADL2 gene or a portion thereof. In some embodiments, the RAI14-RET fusion nucleic acid molecule of the disclosure comprises, in 5′ to 3′ direction, a RAI14 gene or a portion thereof fused to a RET gene or a portion thereof. In some embodiments, the ABI3BP-RET fusion nucleic acid molecule of the disclosure comprises, in 5′ to 3′ direction, a ABI3BP gene or a portion thereof fused to a RET gene or a portion thereof. In some embodiments, the LINC00379-RET fusion nucleic acid molecule of the disclosure comprises, in the 5′ to 3′ direction, a LINC00379 gene or a portion thereof fused to a RET gene or a portion thereof. In some embodiments, the OPTN-RET fusion nucleic acid molecule of the disclosure comprises, in the 5′ to 3′ direction, a OPTN gene or a portion thereof fused to a RET gene or a portion thereof. In some embodiments, the SH2D3A-RET fusion nucleic acid molecule of the disclosure comprises, in the 5′ to 3′ direction, a SH2D3A gene or a portion thereof fused to a RET gene or a portion thereof. In some embodiments, the ZNF721-RET fusion nucleic acid molecule of the disclosure comprises, in the 5′ to 3′ direction, a ZNF721 gene or a portion thereof fused to a RET gene or a portion thereof. In some embodiments, the ADAMTS14-RET fusion nucleic acid molecule of the disclosure comprises, in 5′ to 3′ direction, a ADAMTS14 gene or a portion thereof fused to a RET gene or a portion thereof. In some embodiments, the CSGALNACT2-RET fusion nucleic acid molecule of the disclosure comprises, in 5′ to 3′ direction, a CSGALNACT2 gene or a portion thereof fused to a RET gene or a portion thereof. In some embodiments, the CPEB3-RET fusion nucleic acid molecule of the disclosure comprises, in 5′ to 3′ direction, a CPEB3 gene or a portion thereof fused to a RET gene or a portion thereof. In some embodiments, the RET-CCNY fusion nucleic acid molecule of the disclosure comprises, in 5′ to 3′ direction, a RET gene or a portion thereof fused to a CCNY gene or a portion thereof. In some embodiments, the RET-RASGEF1A fusion nucleic acid molecule of the disclosure comprises, in 5′ to 3′ direction, a RET gene or a portion thereof fused to a RASGEF1A gene or a portion thereof. In some embodiments, the RET-HSD17B7P2 fusion nucleic acid molecule of the disclosure comprises, in 5′ to 3′ direction, a RET gene or a portion thereof fused to a HSD17B7P2 gene or a portion thereof. In some embodiments, the RET-RAD1 fusion nucleic acid molecule of the disclosure comprises, in 5′ to 3′ direction, a RET gene or a portion thereof fused to a RAD1 gene or a portion thereof. In some embodiments, the RET-VSTM4 fusion nucleic acid molecule of the disclosure comprises, in 5′ to 3′ direction, a RET gene or a portion thereof fused to a VSTM4 gene or a portion thereof. In some embodiments, the ANKRD26-RET fusion nucleic acid molecule of the disclosure comprises, in 5′ to 3′ direction, an ANKRD26 gene or a portion thereof fused to a RET gene or a portion thereof. In some embodiments, the CLIP1-RET fusion nucleic acid molecule of the disclosure comprises, in 5′ to 3′ direction, a CLIP1 gene or a portion thereof fused to a RET gene or a portion thereof. In some embodiments, the DLG5-RET fusion nucleic acid molecule of the disclosure comprises, in 5′ to 3′ direction, a DLG5 gene or a portion thereof fused to a RET gene or a portion thereof. In some embodiments, the ERC1-RET fusion nucleic acid molecule of the disclosure comprises, in 5′ to 3′ direction, an ERC1 gene or a portion thereof fused to a RET gene or a portion thereof. In some embodiments, the FRMD4A-RET fusion nucleic acid molecule of the disclosure comprises, in 5′ to 3′ direction, a FRMD4A gene or a portion thereof fused to a RET gene or a portion thereof. In some embodiments, the KIAA1468-RET fusion nucleic acid molecule of the disclosure comprises, in 5′ to 3′ direction, a KIAA1468 gene or a portion thereof fused to a RET gene or a portion thereof. In some embodiments, the NCOA4-RET fusion nucleic acid molecule of the disclosure comprises, in 5′ to 3′ direction, a NCOA4 gene or a portion thereof fused to a RET gene or a portion thereof. In some embodiments, the PARD3-RET fusion nucleic acid molecule of the disclosure comprises, in 5′ to 3′ direction, a PARD3 gene or a portion thereof fused to a RET gene or a portion thereof. In some embodiments, the PRKAR1A-RET fusion nucleic acid molecule of the disclosure comprises, in 5′ to 3′ direction, a PRKAR1A gene or a portion thereof fused to a RET gene or a portion thereof. In some embodiments, the PRKG1-RET fusion nucleic acid molecule of the disclosure comprises, in 5′ to 3′ direction, a PRKG1 gene or a portion thereof fused to a RET gene or a portion thereof. In some embodiments, the RUFY2-RET fusion nucleic acid molecule of the disclosure comprises, in 5′ to 3′ direction, a RUFY2 gene or a portion thereof fused to a RET gene or a portion thereof. In some embodiments, the SNRNP70-RET fusion nucleic acid molecule of the disclosure comprises, in 5′ to 3′ direction, a SNRNP70 gene or a portion thereof fused to a RET gene or a portion thereof. In some embodiments, the SQSTM1-RET fusion nucleic acid molecule of the disclosure comprises, in 5′ to 3′ direction, a SQSTM1 gene or a portion thereof fused to a RET gene or a portion thereof. In some embodiments, the TNIP1-RET fusion nucleic acid molecule of the disclosure comprises, in 5′ to 3′ direction, a TNIP1 gene or a portion thereof fused to a RET gene or a portion thereof. In some embodiments, the TRIM27-RET fusion nucleic acid molecule of the disclosure comprises, in 5′ to 3′ direction, a TRIM27 gene or a portion thereof fused to a RET gene or a portion thereof. In some embodiments, the TRIM33-RET fusion nucleic acid molecule of the disclosure comprises, in 5′ to 3′ direction, a TRIM33 gene or a portion thereof fused to a RET gene or a portion thereof. In some embodiments, the CCDC6-RET fusion nucleic acid molecule of the disclosure comprises, in 5′ to 3′ direction, a CCDC6 gene or a portion thereof fused to a RET gene or a portion thereof. In some embodiments, the TRIM24-RET fusion nucleic acid molecule of the disclosure comprises, in 5′ to 3′ direction, a TRIM24 gene or a portion thereof fused to a RET gene or a portion thereof. In some embodiments, the FGFR1OP-RET fusion nucleic acid molecule of the disclosure comprises, in 5′ to 3′ direction, a FGFR1OP gene or a portion thereof fused to a RET gene or a portion thereof. In some embodiments, the GAS2-RET fusion nucleic acid molecule of the disclosure comprises, in 5′ to 3′ direction, a GAS2 gene or a portion thereof fused to a RET gene or a portion thereof. In some embodiments, the HOOK1-RET fusion nucleic acid molecule of the disclosure comprises, in 5′ to 3′ direction, a HOOK1 gene or a portion thereof fused to a RET gene or a portion thereof. In some embodiments, the LMNA-RET fusion nucleic acid molecule of the disclosure comprises, in 5′ to 3′ direction, a LMNA gene or a portion thereof fused to a RET gene or a portion thereof. In some embodiments, the MLPH-RET fusion nucleic acid molecule of the disclosure comprises, in 5′ to 3′ direction, a MLPH gene or a portion thereof fused to a RET gene or a portion thereof. In some embodiments, the MYH9-RET fusion nucleic acid molecule of the disclosure comprises, in 5′ to 3′ direction, a MYH9 gene or a portion thereof fused to a RET gene or a portion thereof. In some embodiments, the PCDH15-RET fusion nucleic acid molecule of the disclosure comprises, in 5′ to 3′ direction, a PCDH15 gene or a portion thereof fused to a RET gene or a portion thereof. In some embodiments, the PIBF1-RET fusion nucleic acid molecule of the disclosure comprises, in 5′ to 3′ direction, a PIBF1 gene or a portion thereof fused to a RET gene or a portion thereof. In some embodiments, the SLC12A2-RET fusion nucleic acid molecule of the disclosure comprises, in 5′ to 3′ direction, a SLC12A2 gene or a portion thereof fused to a RET gene or a portion thereof. In some embodiments, the ZNF485-RET fusion nucleic acid molecule of the disclosure comprises, in the 5′ to 3′ direction, a ZNF485 gene or a portion thereof fused to a RET gene or a portion thereof.

In some embodiments, a RET fusion nucleic acid molecule of the disclosure comprises or results from a 5′ breakpoint and/or a 3′ breakpoint within the corresponding exons or introns as indicated in Table 4.

TABLE 4
Exonic/intronic breakpoints of RET fusion nucleic acid molecules.

| RET Fusion Nucleic | | |
|---|---|---|
| Acid Molecule | 5′ Breakpoint | 3′ Breakpoint |
| GOLGB1 - RET | GOLGB1 intron 10 | RET intron 11 |
| GP2 - RET | GP2 intron 4 | RET intron 10 |
| PPP2R5A - RET | PPP2R5A intron 1 | RET intron 19 |
| GRM7 - RET | GRM7 intron 9 | RET intron 2 |
| MYH14 - RET | MYH14 intron 37 | RET intron 11 |
| MGEA5 - RET | MGEA5 intron 12 | RET intron 11 |
| SAMD4A - RET | SAMD4A intron 2 | RET intron 10 |
| ACPP - RET | ACPP intron 10 | RET intron 10 |
| BMS1 - RET | BMS1 intron 13 | RET intron 9 |
| CEP135 - RET | CEP135 intron 11 | RET intron 11 |
| EEA1 - RET | EEA1 intron 13 | RET intron 11 |
| CSGALNACT2 - RET | CSGALNACT2 intron 6 | RET intron 11 |
| KIAA1217 - RET | KIAA1217 intron 11 | RET intron 7 |
| SORBS1 - RET | SORBS1 intron 20 | RET intron 6 |
| SORBS1 - RET | SORBS1 intron 22 | RET intron 10 |
| MPRIP - RET | MPRIP intron 22 | RET intron 11 |
| TFG - RET | TFG intron 5 | RET intron 10 |
| SPECC1L - RET | SPECC1L intron 10 | RET intron 11 |
| REEP3 - RET | REEP3 intron 5 | RET intron 11 |
| RRBP1 - RET | RRBP1 intron 22 | RET intron 11 |
| ETV6 - RET | ETV6 intron 6 | RET intron 11 |
| TAF3 - RET | TAF3 intron 3 | RET intron 11 |
| PCM1 - RET | PCM1 intron 29 | RET intron 11 |
| TNIP2 - RET | TNIP2 intron 5 | RET intron 11 |
| SATB1 - RET | SATB1 intron 7 | RET intron 11 |
| RET - ADCY1 | RET intron 11 | ADCY1 intron 11 |
| RET - ZNF248 | RET intron 11 | ZNF248 intron 3 |
| RET - AGBL4 | RET intron 12 | AGBL4 intron 2 |
| RET - LRMDA | RET intron 10 | LRMDA intron 5 |
| RET - ARHGAP19 | RET intron 11 | ARHGAP19 intron 6 |
| RET - CPEB3 | RET intron 11 | CPEB3 intron 7 |
| RET - DCC | RET intron 9 | DCC intron 7 |
| RET - ELMO1 | RET intron 11 | ELMO1 intron 13 |
| RET - WDFY4 | RET intron 15 | WDFY4 intron 39 |
| MKX - RET | MKX intron 5 | RET intron 11 |
| RBMS3 - RET | RBMS3 intron 6 | RET intron 11 |
| SGIP1 - RET | SGIP1 intron 1 | RET intron 11 |
| ZSWIM6 - RET | ZSWIM6 intron 1 | RET intron 11 |
| ALOX5 - RET | ALOX5 intron 2 | RET intron 11 |
| RET - NTRK2 | RET intron 10 | NTRK2 intron 19 |
| RET - OXR1 | RET intron 11 | OXR1 intron 3 |
| RET - CCBE1 | RET intron 8 | CCBE1 intron 2 |
| RET - NAALADL2 | RET intron 11 | NAALADL2 intron 12 |
| RAI14 - RET | RAI14 intron 15 | RET exon 11 |
| ABI3BP - RET | ABI3BP exon 35 | RET intron 11 |
| LINC00379 - RET | LINC00379 intron 2 | RET intron 11 |
| OPTN - RET | OPTN intron 1 | RET intron 10 |
| SH2D3A - RET | SH2D3A intron 1 | RET intron 11 |
| ZNF721 - RET | ZNF721 intron 1 | RET intron 11 |
| ADAMTS14 - RET | ADAMTS14 intron 4 | RET intron 11 |
| CSGALNACT2 - RET | CSGALNACT2 intron 1 | RET intron 11 |
| CPEB3 - RET | CPEB3 exon 10 | RET intron 11 |
| RET - CCNY | RET intron 11 | CCNY intron 3 |
| RET - RASGEF1A | RET intron 5 | RASGEF1A intron 1 |
| RET - HSD17B7P2 | RET exon 11 | HSD17B7P2 exon 8 |
| RET - RAD1 | RET exon 11 | RAD1 exon 6 |
| RET - VSTM4 | RET intron 10 | VSTM4 exon 8 |
| ANKRD26 - RET | ANKRD26 intron 29 | RET intron 11 |
| CLIP1 - RET | CLIP1 intron 16 | RET intron 11 |
| DLG5 - RET | DLG5 intron 14 | RET intron 11 |
| DLG5 - RET | DLG5 intron 27 | RET intron 7 |
| ERC1 - RET | ERC1 intron 12 | RET intron 11 |
| ERC1 - RET | ERC1 intron 8 | RET intron 11 |
| ERC1 - RET | ERC1 intron 12 | RET intron 10 |
| FRMD4A - RET | FRMD4A intron 12 | RET intron 11 |
| KIAA1468 - RET | KIAA1468 intron 10 | RET intron 11 |
| NCOA4 - RET | NCOA4 intron 8 | RET intron 11 |
| PARD3 - RET | PARD3 intron 2 | RET intron 7 |
| PARD3 - RET | PAR3 intron 4 | RET intron 11 |
| PRKAR1A - RET | PRKAR1A intron 7 | RET intron 11 |
| PRKG1 - RET | PRKG1 intron 7 | RET intron 3 |
| PRKG1 - RET | PRKG1 intron 5 | RET intron 17 |
| RUFY2 - RET | RUFY2 intron 9 | RET intron 11 |
| SNRNP70 - RET | SNRNP70 intron 6 | RET intron 11 |
| SQSTM1 - RET | SQSTM1 intron 5 | RET intron 10 |
| TNIP1 - RET | TNIP1 intron 13 | RET intron 11 |
| TNIP1 - RET | TNIP1 intron 15 | RET intron 11 |
| TNIP1 - RET | TNIP1 intron 12 | RET intron 11 |
| TRIM27 - RET | TRIM27 intron 3 | RET intron 11 |
| TRIM33 - RET | TRIM33 intron 10 | RET intron 11 |
| CCDC6 - RET | CCDC6 intron 3 | RET intron 10 |
| TRIM24 - RET | TRIM24 intron 17 | RET intron 11 |
| FGFR1OP-RET | FGFR1OP intron 6 | RET intron 11 |
| GAS2-RET | GAS2 intron 6 | RET intron 3 |
| HOOK1-RET | HOOK1 intron 20 | RET intron 11 |
| LMNA-RET | LMNA intron 2 | RET intron 11 |
| MLPH-RET | MLPH intron 9 | RET intron 11 |
| MYH9-RET | MYH9 intron 34 | RET intron 11 |
| PCDH15-RET | PCDH15 intron 1 | RET intron 4 |
| PIBF1-RET | PIBF1 intron 16 | RET intron 11 |
| SLC12A2-RET | SLC12A2 intron 16 | RET intron 11 |
| ZNF485-RET | ZNF485 intron 4 | RET intron 11 |
| ZNF485-RET | ZNF485 intron 4 | RET intron 9 |

In some embodiments, the GOLGB1-RET fusion nucleic acid molecule of the disclosure comprises or results from a 5′ breakpoint within GOLGB1 intron 10 and/or a 3′ breakpoint within RET intron 11. In some embodiments, the GP2-RET fusion nucleic acid molecule of the disclosure comprises or results from a 5′ breakpoint within GP2 intron 4 and/or a 3′ breakpoint within RET intron 10. In some embodiments, the PPP2R5A-RET fusion nucleic acid molecule of the disclosure comprises or results from a 5′ breakpoint within PPP2R5A intron 1 and/or a 3′ breakpoint within RET intron 19. In some embodiments, the GRM7-RET fusion nucleic acid molecule of the disclosure comprises or results from a 5′ breakpoint within GRM7 intron 9 and/or a 3′ breakpoint within RET intron 2. In some embodiments, the MYH14-RET fusion nucleic acid molecule of the disclosure comprises or results from a 5′ breakpoint within MYH14 intron 37 and/or a 3′ breakpoint within RET intron 11. In some embodiments, the MGEA5-RET fusion nucleic acid molecule of the disclosure comprises or results from a 5′ breakpoint within MGEA5 intron 12 and/or a 3′ breakpoint within RET intron 11. In some embodiments, the SAMD4A-RET fusion nucleic acid molecule of the disclosure comprises or results from a 5′ breakpoint within SAMD4A intron 2 and/or a 3′ breakpoint within RET intron 10. In some embodiments, the ACPP-RET fusion nucleic acid molecule of the disclosure comprises or results from a 5′ breakpoint within ACPP intron 10 and/or a 3′ breakpoint within RET intron 10. In some embodiments, the BMS1-RET fusion nucleic acid molecule of the disclosure comprises or results from a 5′ breakpoint within BMS1 intron 13 and/or a 3′ breakpoint within RET intron 9. In some embodiments, the CEP135-RET fusion nucleic acid molecule of the disclosure comprises or results from a 5′ breakpoint within CEP135 intron 11 and/or a 3′ breakpoint within RET intron 11. In some embodiments, the EEA1-RET fusion nucleic acid molecule of the disclosure comprises or results from a 5′ breakpoint within EEA1 intron 13 and/or a 3′ breakpoint within RET intron 11. In some embodiments, the CSGALNACT2-RET fusion nucleic acid molecule of the disclosure comprises or results from a 5′ breakpoint within CSGALNACT2 intron 6 and/or a 3′ breakpoint within RET intron 11. In some embodiments, the KIAA1217-RET fusion nucleic acid molecule of the disclosure comprises or results from a 5′ breakpoint within KIAA1217 intron 11 and/or a 3′ breakpoint within RET intron 7. In some embodiments, the SORBS1-RET fusion nucleic acid molecule of the disclosure comprises or results from a 5′ breakpoint within SORBS1 intron 20 and/or a 3′ breakpoint within RET intron 6. In some embodiments, the SORBS1-RET fusion nucleic acid molecule of the disclosure comprises or results from a 5′ breakpoint within SORBS1 intron 22 and/or a 3′ breakpoint within RET intron 10. In some embodiments, the MPRIP-RET fusion nucleic acid molecule of the disclosure comprises or results from a 5′ breakpoint within MPRIP intron 22 and/or a 3′ breakpoint within RET intron 11. In some embodiments, the TFG-RET fusion nucleic acid molecule of the disclosure comprises or results from a 5′ breakpoint within TFG intron 5 and/or a 3′ breakpoint within RET intron 10. In some embodiments, the SPECC1L-RET fusion nucleic acid molecule of the disclosure comprises or results from a 5′ breakpoint within SPECC1L intron 10 and/or a 3′ breakpoint within RET intron 11. In some embodiments, the REEP3-RET fusion nucleic acid molecule of the disclosure comprises or results from a 5′ breakpoint within REEP3 intron 5 and/or a 3′ breakpoint within RET intron 11. In some embodiments, the RRBP1-RET fusion nucleic acid molecule of the disclosure comprises or results from a 5′ breakpoint within RRBP1 intron 22 and/or a 3′ breakpoint within RET intron 11. In some embodiments, the ETV6-RET fusion nucleic acid molecule of the disclosure comprises or results from a 5′ breakpoint within ETV6 intron 6 and/or a 3′ breakpoint within RET intron 11. In some embodiments, the TAF3-RET fusion nucleic acid molecule of the disclosure comprises or results from a 5′ breakpoint within TAF3 intron 3 and/or a 3″ breakpoint within RET intron 11. In some embodiments, the PCM1-RET fusion nucleic acid molecule of the disclosure comprises or results from a 5′ breakpoint within PCM1 intron 29 and/or a 3′ breakpoint within RET intron 11. In some embodiments, the TNIP2-RET fusion nucleic acid molecule of the disclosure comprises or results from a 5′ breakpoint within TNIP2 intron 5 and/or a 3′ breakpoint within RET intron 11. In some embodiments, the SATB1-RET fusion nucleic acid molecule of the disclosure comprises or results from a 5′ breakpoint within SATB1 intron 7 and/or a 3′ breakpoint within RET intron 11. In some embodiments, the RET-ADCY1 fusion nucleic acid molecule of the disclosure comprises or results from a 5′ breakpoint within RET intron 11 and/or a 3″ breakpoint within ADCY1 intron 11. In some embodiments, the RET-ZNF248 fusion nucleic acid molecule of the disclosure comprises or results from a 5′ breakpoint within RET intron 11 and/or a 3″ breakpoint within ZNF248 intron 3. In some embodiments, the RET-AGBL4 fusion nucleic acid molecule of the disclosure comprises or results from a 5′ breakpoint within RET intron 12 and/or a 3′ breakpoint within AGBL4 intron 2. In some embodiments, the RET-LRMDA fusion nucleic acid molecule of the disclosure comprises or results from a 5′ breakpoint within RET intron 10 and/or a 3″ breakpoint within LRMDA intron 5. In some embodiments, the RET-ARHGAP19 fusion nucleic acid molecule of the disclosure comprises or results from a 5′ breakpoint within RET intron 11 and/or a 3′ breakpoint within ARHGAP19 intron 6. In some embodiments, the RET-CPEB3 fusion nucleic acid molecule of the disclosure comprises or results from a 5′ breakpoint within RET intron 11 and/or a 3″ breakpoint within CPEB3 intron 7. In some embodiments, the RET-DCC fusion nucleic acid molecule of the disclosure comprises or results from a 5′ breakpoint within RET intron 9 and/or a 3′ breakpoint within DCC intron 7. In some embodiments, the RET-ELMO1 fusion nucleic acid molecule of the disclosure comprises or results from a 5′ breakpoint within RET intron 11 and/or a 3′ breakpoint within ELMO1 intron 13. In some embodiments, the RET-WDFY4 fusion nucleic acid molecule of the disclosure comprises or results from a 5′ breakpoint within RET intron 15 and/or a 3′ breakpoint within WDFY4 intron 39. In some embodiments, the MKX-RET fusion nucleic acid molecule of the disclosure comprises or results from a 5′ breakpoint within MKX intron 5 and/or a 3″ breakpoint within RET intron 11. In some embodiments, the RBMS3-RET fusion nucleic acid molecule of the disclosure comprises or results from a 5′ breakpoint within RBMS3 intron 6 and/or a 3′ breakpoint within RET intron 11. In some embodiments, the SGIP1-RET fusion nucleic acid molecule of the disclosure comprises or results from a 5′ breakpoint within SGIP1 intron 1 and/or a 3′ breakpoint within RET intron 11. In some embodiments, the ZSWIM6-RET fusion nucleic acid molecule of the disclosure comprises or results from a 5′ breakpoint within ZSWIM6 intron 1 and/or a 3′ breakpoint within RET intron 11. In some embodiments, the ALOX5-RET fusion nucleic acid molecule of the disclosure comprises or results from a 5′ breakpoint within ALOX5 intron 2 and/or a 3′ breakpoint within RET intron 11. In some embodiments, the RET-NTRK2 fusion nucleic acid molecule of the disclosure comprises or results from a 5′ breakpoint within RET intron 10 and/or a 3′ breakpoint within NTRK2 intron 19. In some embodiments, the RET-OXR1 fusion nucleic acid molecule of the disclosure comprises or results from a 5′ breakpoint within RET intron 11 and/or a 3′ breakpoint within OXR1 intron 3. In some embodiments, the RET-CCBE1 fusion nucleic acid molecule of the disclosure comprises or results from a 5′ breakpoint within RET intron 8 and/or a 3′ breakpoint within CCBE1 intron 2. In some embodiments, the RET-NAALADL2 fusion nucleic acid molecule of the disclosure comprises or results from a 5′ breakpoint within RET intron 11 and/or a 3′ breakpoint within NAALADL2 intron 12. In some embodiments, the RAI14-RET fusion nucleic acid molecule of the disclosure comprises or results from a 5′ breakpoint within RAI14 intron 15 and/or a 3′ breakpoint within RET exon 11. In some embodiments, the ABI3BP-RET fusion nucleic acid molecule of the disclosure comprises or results from a 5′ breakpoint within ABI3BP exon 35 and/or a 3′ breakpoint within RET intron 11. In some embodiments, the LINC00379-RET fusion nucleic acid molecule of the disclosure comprises or results from a 5′ breakpoint within LINC00379 intron 2 and/or a 3′ breakpoint within RET intron 11. In some embodiments, the OPTN-RET fusion nucleic acid molecule of the disclosure comprises or results from a 5′ breakpoint within OPTN intron 1 and/or a 3′ breakpoint within RET intron 10. In some embodiments, the SH2D3A-RET fusion nucleic acid molecule of the disclosure comprises or results from a 5′ breakpoint within SH2D3A intron 1 and/or a 3′ breakpoint within RET intron 11. In some embodiments, the ZNF721-RET fusion nucleic acid molecule of the disclosure comprises or results from a 5′ breakpoint within ZNF721 intron 1 and/or a 3′ breakpoint within RET intron 11. In some embodiments, the ADAMTS14-RET fusion nucleic acid molecule of the disclosure comprises or results from a 5′ breakpoint within ADAMTS14 intron 4 and/or a 3′ breakpoint within RET intron 11. In some embodiments, the CSGALNACT2-RET fusion nucleic acid molecule of the disclosure comprises or results from a 5′ breakpoint within CSGALNACT2 intron 1 and/or a 3′ breakpoint within RET intron 11. In some embodiments, the CPEB3-RET fusion nucleic acid molecule of the disclosure comprises or results from a 5′ breakpoint within CPEB3 exon 10 and/or a 3′ breakpoint within RET intron 11. In some embodiments, the RET-CCNY fusion nucleic acid molecule of the disclosure comprises or results from a 5′ breakpoint within RET intron 11 and/or a 3′ breakpoint within CCNY intron 3. In some embodiments, the RET-RASGEF1A fusion nucleic acid molecule of the disclosure comprises or results from a 5′ breakpoint within RET intron 5 and/or a 3′ breakpoint within RASGEF1A intron 1. In some embodiments, the RET-HSD17B7P2 fusion nucleic acid molecule of the disclosure comprises or results from a 5′ breakpoint within RET exon 11 and/or a 3′ breakpoint within HSD17B7P2 exon 8. In some embodiments, the RET-RAD1 fusion nucleic acid molecule of the disclosure comprises or results from a 5′ breakpoint within RET exon 11 and/or a 3′ breakpoint within RAD1 exon 6. In some embodiments, the RET-VSTM4 fusion nucleic acid molecule of the disclosure comprises or results from a 5′ breakpoint within RET intron 10 and/or a 3′ breakpoint within VSTM4 exon 8. In some embodiments, the ANKRD26-RET fusion nucleic acid molecule of the disclosure comprises or results from a 5′ breakpoint within ANKRD26 intron 29 and/or a 3′ breakpoint within RET intron 11. In some embodiments, the CLIP1-RET fusion nucleic acid molecule of the disclosure comprises or results from a 5′ breakpoint within CLIP1 intron 16 and/or a 3′ breakpoint within RET intron 11. In some embodiments, the DLG5-RET fusion nucleic acid molecule of the disclosure comprises or results from a 5′ breakpoint within DLG5 intron 14 and/or a 3′ breakpoint within RET intron 11. In some embodiments, the DLG5-RET fusion nucleic acid molecule of the disclosure comprises or results from a 5′ breakpoint within DLG5 intron 27 and/or a 3′ breakpoint within RET intron 7. In some embodiments, the ERC1-RET fusion nucleic acid molecule of the disclosure comprises or results from a 5′ breakpoint within ERC1 intron 12 and/or a 3′ breakpoint within RET intron 11. In some embodiments, the ERC1-RET fusion nucleic acid molecule of the disclosure comprises or results from a 5′ breakpoint within ERC1 intron 8 and/or a 3′ breakpoint within RET intron 11. In some embodiments, the ERC1-RET fusion nucleic acid molecule of the disclosure comprises or results from a 5′ breakpoint within ERC1 intron 12 and/or a 3′ breakpoint within RET intron 10. In some embodiments, the FRMD4A-RET fusion nucleic acid molecule of the disclosure comprises or results from a 5′ breakpoint within FRMD4A intron 12 and/or a 3′ breakpoint within RET intron 11. In some embodiments, the KIAA1468-RET fusion nucleic acid molecule of the disclosure comprises or results from a 5′ breakpoint within KIAA1468 intron 10 and/or a 3′ breakpoint within RET intron 11. In some embodiments, the NCOA4-RET fusion nucleic acid molecule of the disclosure comprises or results from a 5′ breakpoint within NCOA4 intron 8 and/or a 3′ breakpoint within RET intron 11. In some embodiments, the PARD3-RET fusion nucleic acid molecule of the disclosure comprises or results from a 5′ breakpoint within PARD3 intron 2 and/or a 3′ breakpoint within RET intron 7. In some embodiments, the PARD3-RET fusion nucleic acid molecule of the disclosure comprises or results from a 5′ breakpoint within PARD3 intron 4 and/or a 3′ breakpoint within RET intron 11. In some embodiments, the PRKAR1A-RET fusion nucleic acid molecule of the disclosure comprises or results from a 5′ breakpoint within PRKAR1A intron 7 and/or a 3′ breakpoint within RET intron 11. In some embodiments, the PRKG1-RET fusion nucleic acid molecule of the disclosure comprises or results from a 5′ breakpoint within PRKG1 intron 7 and/or a 3′ breakpoint within RET intron 3. In some embodiments, the PRKG1-RET fusion nucleic acid molecule of the disclosure comprises or results from a 5′ breakpoint within PRKG1 intron 5 and/or a 3′ breakpoint within RET intron 17. In some embodiments, the RUFY2-RET fusion nucleic acid molecule of the disclosure comprises or results from a 5′ breakpoint within RUFY2 intron 9 and/or a 3′ breakpoint within RET intron 11. In some embodiments, the SNRNP70-RET fusion nucleic acid molecule of the disclosure comprises or results from a 5′ breakpoint within SNRNP70 intron 6 and/or a 3′ breakpoint within RET intron 11. In some embodiments, the SQSTM1-RET fusion nucleic acid molecule of the disclosure comprises or results from a 5′ breakpoint within SQSTM1 intron 5 and/or a 3′ breakpoint within RET intron 10. In some embodiments, the TNIP1-RET fusion nucleic acid molecule of the disclosure comprises or results from a 5′ breakpoint within TNIP1 intron 13 and/or a 3′ breakpoint within RET intron 11. In some embodiments, the TNIP1-RET fusion nucleic acid molecule of the disclosure comprises or results from a 5′ breakpoint within TNIP1 intron 15 and/or a 3′ breakpoint within RET intron 11. In some embodiments, the TNIP1-RET fusion nucleic acid molecule of the disclosure comprises or results from a 5′ breakpoint within TNIP1 intron 12 and/or a 3′ breakpoint within RET intron 11. In some embodiments, the TRIM27-RET fusion nucleic acid molecule of the disclosure comprises or results from a 5′ breakpoint within TRIM27 intron 3 and/or a 3′ breakpoint within RET intron 11. In some embodiments, the TRIM33-RET fusion nucleic acid molecule of the disclosure comprises or results from a 5′ breakpoint within TRIM33 intron 10 and/or a 3′ breakpoint within RET intron 11. In some embodiments, the CCDC6-RET fusion nucleic acid molecule of the disclosure comprises or results from a 5′ breakpoint within CCDC6 intron 3 and/or a 3′ breakpoint within RET intron 10. In some embodiments, the TRIM24-RET fusion nucleic acid molecule of the disclosure comprises or results from a 5′ breakpoint within TRIM24 intron 17 and/or a 3′ breakpoint within RET intron 11. In some embodiments, the FGFR1OP-RET fusion nucleic acid molecule of the disclosure comprises or results from a 5′ breakpoint within FGFR1OP intron 6 and/or a 3′ breakpoint within RET intron 11. In some embodiments, the GAS2-RET fusion nucleic acid molecule of the disclosure comprises or results from a 5′ breakpoint within GAS2 intron 6 and/or a 3′ breakpoint within RET intron 3. In some embodiments, the HOOK1-RET fusion nucleic acid molecule of the disclosure comprises or results from a 5′ breakpoint within HOOK1 intron 20 and/or a 3′ breakpoint within RET intron 11. In some embodiments, the LMNA-RET fusion nucleic acid molecule of the disclosure comprises or results from a 5′ breakpoint within LMNA intron 2 and/or a 3′ breakpoint within RET intron 11. In some embodiments, the MLPH-RET fusion nucleic acid molecule of the disclosure comprises or results from a 5′ breakpoint within MLPH intron 9 and/or a 3′ breakpoint within RET intron 11. In some embodiments, the MYH9-RET fusion nucleic acid molecule of the disclosure comprises or results from a 5′ breakpoint within MYH9 intron 34 and/or a 3′ breakpoint within RET intron 11. In some embodiments, the PCDH15-RET fusion nucleic acid molecule of the disclosure comprises or results from a 5′ breakpoint within PCDH15 intron 1 and/or a 3′ breakpoint within RET intron 4. In some embodiments, the PIBF1-RET fusion nucleic acid molecule of the disclosure comprises or results from a 5′ breakpoint within PIBF1 intron 16 and/or a 3′ breakpoint within RET intron 11. In some embodiments, the SLC12A2-RET fusion nucleic acid molecule of the disclosure comprises or results from a 5′ breakpoint within SLC12A2 intron 16 and/or a 3′ breakpoint within RET intron 11. In some embodiments, the ZNF485-RET fusion nucleic acid molecule of the disclosure comprises or results from a 5′ breakpoint within ZNF485 intron 4 and/or a 3′ breakpoint within RET intron 11. In some embodiments, the ZNF485-RET fusion nucleic acid molecule of the disclosure comprises or results from a 5′ breakpoint within ZNF485 intron 4 and/or a 3′ breakpoint within RET intron 9.

In some embodiments, a RET fusion nucleic acid molecule of the disclosure comprises or results from a 5′ breakpoint and/or a 3′ breakpoint within the corresponding chromosomal coordinates as indicated in Table 5.

TABLE 5
Chromosomal coordinates of breakpoints of exemplary
RET fusion nucleic acid molecules.

| RET Fusion Nucleic | | |
|---|---|---|
| Acid Molecule | 5′ breakpoint | 3′ breakpoint |
| GOLGB1 - RET | chr3: 121428577-121428818 | chr10: 43611900-43612184 |
| GP2 - RET | chr16: 20331978-20332315 | chr10: 43609279-43609727 |
| PPP2R5A - RET | chr1: 212474140-212474319 | chr10: 43623512-43623678 |
| GRM7 - RET | chr3: 7728019-7728192 | chr10: 43595834-43596313 |
| MYH14 - RET | chr19: 50801162-50801465 | chr10: 43610285-43610748 |
| MGEA5 - RET | chr10: 103551711-103551879 | chr10: 43610920-43611146 |
| SAMD4A - RET | chr14: 55123072-55123234 | chr10: 43609336-43609516 |
| ACPP - RET | chr3: 132078976-132079163 | chr10: 43609901-43610079 |
| BMS1 - RET | chr10: 43307628-43307807 | chr10: 43608772-43609039 |
| CEP135 - RET | chr4: 56844220-56844527 | chr10: 43611070-43611610 |
| EEA1 - RET | chr12: 93218451-93218763 | chr10: 43611264-43611592 |
| CSGALNACT2 - RET | chr10: 43671043-43671216 | chr10: 43611674-43611871 |
| KIAA1217 - RET | chr10: 24810035-24810374 | chr10: 43607337-43607677 |
| SORBS1 - RET | chr10: 97098893-97098933 | chr10: 43606666-43606706 |
| SORBS1 - RET | chr10: 97077294-97077537 | chr10: 43609629-43609972 |
| MPRIP - RET | chr17: 17083314-17083354 | chr10: 43612074-43612114 |
| TFG - RET | chr3: 100453395-100453864 | chr10: 43609629-43610065 |
| SPECC1L - RET | chr22: 24739286-24739616 | chr10: 43611732-43612114 |
| REEP3 - RET | chr10: 65367361-65368009 | chr10: 43611264-43611758 |
| RRBP1 - RET | chr20: 17596315-17596767 | chr10: 43610785-43611339 |
| ETV6 - RET | chr12: 12038351-12038584 | chr10: 43611377-43611586 |
| TAF3 - RET | chr10: 8010776-8011037 | chr10: 43610288-43610507 |
| PCM1 - RET | chr8: 17856613-17856789 | chr10: 43611737-43612079 |
| PCM1 - RET | chr8: 17858876-17859548 | chr10: 43610288-43611131 |
| TNIP2 - RET | chr4: 2745391-2745953 | chr10: 43611294-43612177 |
| SATB1 - RET | chr3: 18433247-18433798 | chr10: 43610364-43611028 |
| RET - ADCY1 | chr10: 43610900-43611038 | chr7: 45724501-45724758 |
| RET - ZNF248 | chr10: 43610329-43610698 | chr10: 38143593-38143957 |
| RET - AGBL4 | chr10: 43612164-43612322 | chr1: 50296066-50296164 |
| RET - LRMDA | chr10: 43609359-43609695 | chr10: 78117734-78117976 |
| RET - ARHGAP19 | chr10: 43610326-43610687 | chr10: 99014615-99014923 |
| RET - CPEB3 | chr10: 43610292-43610477 | chr10: 93857438-93857889 |
| RET - DCC | chr10: 43608372-43608512 | chr18: 50604698-50604780 |
| RET - ELMO1 | chr10: 43611573-43611801 | chr7: 37238470-37238795 |
| RET - WDFY4 | chr10: 43615421-43615713 | chr10: 50051602-50051938 |
| MKX - RET | chr10: 27975034-27975183 | chr10: 43610285-43610466 |
| RBMS3 - RET | chr3: 29823809-29823976 | chr10: 43612021-43612151 |
| SGIP1 - RET | chr1: 67073312-67073582 | chr10: 43611993-43612185 |
| ZSWIM6 - RET | chr5: 60644190-60644600 | chr10: 43611931-43612183 |
| ALOX5 - RET | chr10: 45880054-45880377 | chr10: 43611435-43611795 |
| RET - NTRK2 | chr10: 43609278-43609516 | chr9: 87615294-87615609 |
| RET - OXR1 | chr10: 43611090-43611446 | chr8: 107581829-107582082 |
| RET - CCBE1 | chr10: 43607774-43607874 | chr18: 57343203-57343676 |
| RET - NAALADL2 | chr10: 43611602-43611922 | chr3: 175458489-175458778 |
| RAI14 - RET | chr5: 34825746-34825976 | chr10: 43610042-43610268 |
| ABI3BP - RET | chr3: 100469166-100469527 | chr10: 43610324-43610920 |
| LINC00379 - RET | chr13: 91833445-91833706 | chr10: 43611105-43611505 |
| OPTN - RET | chr10: 13145247-13145480 | chr10: 43609677-43609931 |
| SH2D3A - RET | chr19: 6765967-6766439 | chr10: 43611764-43612170 |
| ZNF721 - RET | chr4: 472119-472287 | chr10: 43610406-43610589 |
| ADAMTS14 - RET | chr10: 72480354 | chr10: 43611720 |
| CSGALNACT2 - RET | chr10: 43648115-43648300 | chr10: 43610689-43610896 |
| CPEB3 - RET | chr10: 93808581-93808772 | chr10: 43611585-43611862 |
| RET - CCNY | chr10: 43611301-43611576 | chr10: 35549015-35549283 |
| RET - RASGEF1A | chr10: 43601756-43602031 | chr10: 43709383-43709498 |
| RET - HSD17B7P2 | chr10: 43609878-43609957 | chr10: 38667312-38667485 |
| RET - RAD1 | chr10: 43609716-43610036 | chr5: 34906366-34906711 |
| RET - VSTM4 | chr10: 43609237-43609437 | chr10: 50225719-50225999 |
| ANKRD26 - RET | chr10: 27310264-27310361 | chr10: 43610383-43610480 |
| CLIP1 - RET | chr12: 122805302-122805302 | chr10: 43611109-43611109 |
| DLG5 - RET | chr10: 79582000-79582000 | chr10: 43611570-43611570 |
| DLG5 - RET | chr10: 79559942-79559942 | chr10: 43607485-43607485 |
| ERC1 - RET | chr12: 1346557-1346557 | chr10: 43610879-43610879 |
| ERC1 - RET | chr12: 1290627-1290627 | chr10: 43611066-43611066 |
| ERC1 - RET | chr12: 1350447-1350447 | chr10: 43609823-43609823 |
| FRMD4A - RET | chr10: 13758406-13758406 | chr10: 43610354-43610354 |
| KIAA1468 - RET | chr18: 59908435-59908435 | chr10: 43611083-43611083 |
| NCOA4 - RET | chr10: 51585637-51585735 | chr10: 43610257-43610403 |
| PARD3 - RET | chr10: 34926445-34926445 | chr10: 43607138-43607138 |
| PARD3 - RET | chr10: 34755707-34755707 | chr10: 43612003-43612003 |
| PRKAR1A - RET | chr17: 66523509-66523607 | chr10: 43611401-43611502 |
| PRKG1 - RET | chr10: 53889856-53889954 | chr10: 43600288-43600402 |
| PRKG1 - RET | chr10: 53789188-53789284 | chr10: 43620162-43620329 |
| RUFY2 - RET | chr10: 70143736-70143833 | chr10: 43611405-43611519 |
| SNRNP70 - RET | chr19: 49602640-49602640 | chr10: 43611323-43611323 |
| SQSTM1 - RET | chr5: 179254189-179254286 | chr10: 43609433-43609563 |
| TNIP1 - RET | chr5: 150416127-150416127 | chr10: 43611669-43611669 |
| TNIP1 - RET | chr5: 150414253-150414351 | chr10: 43611007-43611109 |
| TNIP1 - RET | chr5: 150417592-150417592 | chr10: 43611428-43611428 |
| TRIM27 - RET | chr6: 28881702-28881890 | chr10: 43610709-43610878 |
| TRIM33 - RET | chr1: 114965871-114965970 | chr10: 43611514-43611626 |
| CCDC6 - RET | chr10: 61585545-61585642 | chr10: 43609686-43609783 |
| TRIM24 - RET | chr7: 138267911-138267911 | chr10: 43611642-43611642 |
| FGFR1OP-RET | chr6: 167424630-167424630 | chr10: 43611296-43611296 |
| GAS2-RET | chr11: 22812170-22812170 | chr10: 43600385-43600385 |
| HOOK1-RET | chr1: 60334563-60334563 | chr10: 43610286-43610286 |
| LMNA-RET | chr1: 156102332-156102332 | chr10: 43611407-43611407 |
| LMNA-RET | chr1: 156102293-156102386 | chr10: 43611365-43611462 |
| MLPH-RET | chr2: 238445254-238445254 | chr10: 43610453-43610453 |
| MYH9-RET | chr22: 36683797-36683797 | chr10: 43611848-43611848 |
| PCDH15-RET | chr10: 56509282-56509282 | chr10: 43601819-43601819 |
| PIBF1-RET | chr13: 73550847-73550847 | chr10: 43611532-43611532 |
| PIBF1-RET | chr13: 73548007-73548007 | chr10: 43611167-43611167 |
| SLC12A2-RET | chr5: 127496774-127496774 | chr10: 43611855-43611855 |
| ZNF485-RET | chr10: 44109385-44109456 | chr10: 43611964-43612093 |
| ZNF485-RET | chr10: 44109396-44109396 | chr10: 43608521-43608521 |

In some embodiments, the GOLGB1-RET fusion nucleic acid molecule of the disclosure comprises or results from a breakpoint within chromosomal coordinates chr3: 121428577-121428818 and/or chr10: 43611900-43612184. In some embodiments, the GP2-RET fusion nucleic acid molecule of the disclosure comprises or results from a breakpoint within chromosomal coordinates chr16: 20331978-20332315 and/or chr10: 43609279-43609727. In some embodiments, the PPP2R5A-RET fusion nucleic acid molecule of the disclosure comprises or results from a breakpoint within chromosomal coordinates chr1: 212474140-212474319 and/or chr10: 43623512-43623678. In some embodiments, the GRM7-RET fusion nucleic acid molecule of the disclosure comprises or results from a breakpoint within chromosomal coordinates chr3: 7728019-7728192 and/or chr10: 43595834-43596313. In some embodiments, the MYH14-RET fusion nucleic acid molecule of the disclosure comprises or results from a breakpoint within chromosomal coordinates chr19: 50801162-50801465 and/or chr10: 43610285-43610748. In some embodiments, the MGEA5-RET fusion nucleic acid molecule of the disclosure comprises or results from a breakpoint within chromosomal coordinates chr10: 103551711-103551879 and/or chr10: 43610920-43611146. In some embodiments, the SAMD4A-RET fusion nucleic acid molecule of the disclosure comprises or results from a breakpoint within chromosomal coordinates chr14: 55123072-55123234 and/or chr10: 43609336-43609516. In some embodiments, the ACPP-RET fusion nucleic acid molecule of the disclosure comprises or results from a breakpoint within chromosomal coordinates chr3: 132078976-132079163 and/or chr10: 43609901-43610079. In some embodiments, the BMS1-RET fusion nucleic acid molecule of the disclosure comprises or results from a breakpoint within chromosomal coordinates chr10: 43307628-43307807 and/or chr10: 43608772-43609039. In some embodiments, the CEP135-RET fusion nucleic acid molecule of the disclosure comprises or results from a breakpoint within chromosomal coordinates chr4: 56844220-56844527 and/or chr10: 43611070-43611610. In some embodiments, the EEA1-RET fusion nucleic acid molecule of the disclosure comprises or results from a breakpoint within chromosomal coordinates chr12: 93218451-93218763 and/or chr10: 43611264-43611592. In some embodiments, the CSGALNACT2-RET fusion nucleic acid molecule of the disclosure comprises or results from a breakpoint within chromosomal coordinates chr10: 43671043-43671216 and/or chr10: 43611674-43611871. In some embodiments, the KIAA1217-RET fusion nucleic acid molecule of the disclosure comprises or results from a breakpoint within chromosomal coordinates chr10: 24810035-24810374 and/or chr10: 43607337-43607677. In some embodiments, the SORBS1-RET fusion nucleic acid molecule of the disclosure comprises or results from a breakpoint within chromosomal coordinates chr10: 97098893-97098933 and/or chr10: 43606666-43606706. In some embodiments, the SORBS1-RET fusion nucleic acid molecule of the disclosure comprises or results from a breakpoint within chromosomal coordinates chr10: 97077294-97077537 and/or chr10: 43609629-43609972. In some embodiments, the MPRIP-RET fusion nucleic acid molecule of the disclosure comprises or results from a breakpoint within chromosomal coordinates chr17: 17083314-17083354 and/or chr10: 43612074-43612114. In some embodiments, the TFG-RET fusion nucleic acid molecule of the disclosure comprises or results from a breakpoint within chromosomal coordinates chr3: 100453395-100453864 and/or chr10: 43609629-43610065. In some embodiments, the SPECC1L-RET fusion nucleic acid molecule of the disclosure comprises or results from a breakpoint within chromosomal coordinates chr22: 24739286-24739616 and/or chr10: 43611732-43612114. In some embodiments, the REEP3-RET fusion nucleic acid molecule of the disclosure comprises or results from a breakpoint within chromosomal coordinates chr10: 65367361-65368009 and/or chr10: 43611264-43611758. In some embodiments, the RRBP1-RET fusion nucleic acid molecule of the disclosure comprises or results from a breakpoint within chromosomal coordinates chr20: 17596315-17596767 and/or chr10: 43610785-43611339. In some embodiments, the ETV6-RET fusion nucleic acid molecule of the disclosure comprises or results from a breakpoint within chromosomal coordinates chr12: 12038351-12038584 and/or chr10: 43611377-43611586. In some embodiments, the TAF3-RET fusion nucleic acid molecule of the disclosure comprises or results from a breakpoint within chromosomal coordinates chr10: 8010776-8011037 and/or chr10: 43610288-43610507. In some embodiments, the PCM1-RET fusion nucleic acid molecule of the disclosure comprises or results from a breakpoint within chromosomal coordinates chr8: 17856613-17856789 and/or chr10: 43611737-43612079. In some embodiments, the PCM1-RET fusion nucleic acid molecule of the disclosure comprises or results from a breakpoint within chromosomal coordinates chr8: 17858876-17859548 and/or chr10: 43610288-43611131. In some embodiments, the TNIP2-RET fusion nucleic acid molecule of the disclosure comprises or results from a breakpoint within chromosomal coordinates chr4: 2745391-2745953 and/or chr10: 43611294-43612177. In some embodiments, the SATB1-RET fusion nucleic acid molecule of the disclosure comprises or results from a breakpoint within chromosomal coordinates chr3: 18433247-18433798 and/or chr10: 43610364-43611028. In some embodiments, the RET-ADCY1 fusion nucleic acid molecule of the disclosure comprises or results from a breakpoint within chromosomal coordinates chr10: 43610900-43611038 and/or chr7: 45724501-45724758. In some embodiments, the RET-ZNF248 fusion nucleic acid molecule of the disclosure comprises or results from a breakpoint within chromosomal coordinates chr10: 43610329-43610698 and/or chr10: 38143593-38143957. In some embodiments, the RET-AGBL4 fusion nucleic acid molecule of the disclosure comprises or results from a breakpoint within chromosomal coordinates chr 10:43612164-43612322 and/or chr1: 50296066-50296164. In some embodiments, the RET-LRMDA fusion nucleic acid molecule of the disclosure comprises or results from a breakpoint within chromosomal coordinates chr10: 43609359-43609695 and/or chr10: 78117734-78117976. In some embodiments, the RET-ARHGAP19 fusion nucleic acid molecule of the disclosure comprises or results from a breakpoint within chromosomal coordinates chr10: 43610326-43610687 and/or chr10: 99014615-99014923. In some embodiments, the RET-CPEB3 fusion nucleic acid molecule of the disclosure comprises or results from a breakpoint within chromosomal coordinates chr10: 43610292-43610477 and/or chr10: 93857438-93857889. In some embodiments, the RET-DCC fusion nucleic acid molecule of the disclosure comprises or results from a breakpoint within chromosomal coordinates chr10: 43608372-43608512 and/or chr18: 50604698-50604780. In some embodiments, the RET-ELMO1 fusion nucleic acid molecule of the disclosure comprises or results from a breakpoint within chromosomal coordinates chr10: 43611573-43611801 and/or chr7: 37238470-37238795. In some embodiments, the RET-WDFY4 fusion nucleic acid molecule of the disclosure comprises or results from a breakpoint within chromosomal coordinates chr10: 43615421-43615713 and/or chr10: 50051602-50051938. In some embodiments, the MKX-RET fusion nucleic acid molecule of the disclosure comprises or results from a breakpoint within chromosomal coordinates chr10: 27975034-27975183 and/or chr10: 43610285-43610466. In some embodiments, the RBMS3-RET fusion nucleic acid molecule of the disclosure comprises or results from a breakpoint within chromosomal coordinates chr3: 29823809-29823976 and/or chr10: 43612021-43612151. In some embodiments, the SGIP1-RET fusion nucleic acid molecule of the disclosure comprises or results from a breakpoint within chromosomal coordinates chr1: 67073312-67073582 and/or chr10: 43611993-43612185. In some embodiments, the ZSWIM6-RET fusion nucleic acid molecule of the disclosure comprises or results from a breakpoint within chromosomal coordinates chr5: 60644190-60644600 and/or chr10: 43611931-43612183. In some embodiments, the ALOX5-RET fusion nucleic acid molecule of the disclosure comprises or results from a breakpoint within chromosomal coordinates chr10: 45880054-45880377 and/or chr10: 43611435-43611795. In some embodiments, the RET-NTRK2 fusion nucleic acid molecule of the disclosure comprises or results from a breakpoint within chromosomal coordinates chr10: 43609278-43609516 and/or chr9: 87615294-87615609. In some embodiments, the RET-OXR1 fusion nucleic acid molecule of the disclosure comprises or results from a breakpoint within chromosomal coordinates chr10: 43611090-43611446 and/or chr8: 107581829-107582082. In some embodiments, the RET-CCBE1 fusion nucleic acid molecule of the disclosure comprises or results from a breakpoint within chromosomal coordinates chr10: 43607774-43607874 and/or chr18: 57343203-57343676. In some embodiments, the RET-NAALADL2 fusion nucleic acid molecule of the disclosure comprises or results from a breakpoint within chromosomal coordinates chr10: 43611602-43611922 and/or chr3: 175458489-175458778. In some embodiments, the RAI14-RET fusion nucleic acid molecule of the disclosure comprises or results from a breakpoint within chromosomal coordinates chr5: 34825746-34825976 and/or chr10: 43610042-43610268. In some embodiments, the ABI3BP-RET fusion nucleic acid molecule of the disclosure comprises or results from a breakpoint within chromosomal coordinates chr3: 100469166-100469527 and/or chr10: 43610324-43610920. In some embodiments, the LINC00379-RET fusion nucleic acid molecule of the disclosure comprises or results from a breakpoint within chromosomal coordinates chr13: 91833445-91833706 and/or chr10: 43611105-43611505. In some embodiments, the OPTN-RET fusion nucleic acid molecule of the disclosure comprises or results from a breakpoint within chromosomal coordinates chr10: 13145247-13145480 and/or chr10: 43609677-43609931. In some embodiments, the SH2D3A-RET fusion nucleic acid molecule of the disclosure comprises or results from a breakpoint within chromosomal coordinates chr19: 6765967-6766439 and/or chr10: 43611764-43612170. In some embodiments, the ZNF721-RET fusion nucleic acid molecule of the disclosure comprises or results from a breakpoint within chromosomal coordinates chr4: 472119-472287 and/or chr10: 43610406-43610589. In some embodiments, the ADAMTS14-RET fusion nucleic acid molecule of the disclosure comprises or results from a breakpoint within chromosomal coordinates chr10: 72480354 and/or chr10: 43611720. In some embodiments, the CSGALNACT2-RET fusion nucleic acid molecule of the disclosure comprises or results from a breakpoint within chromosomal coordinates chr10: 43648115-43648300 and/or chr10: 43610689-43610896. In some embodiments, the CPEB3-RET fusion nucleic acid molecule of the disclosure comprises or results from a breakpoint within chromosomal coordinates chr10: 93808581-93808772 and/or chr10: 43611585-43611862. In some embodiments, the RET-CCNY fusion nucleic acid molecule of the disclosure comprises or results from a breakpoint within chromosomal coordinates chr10: 43611301-43611576 and/or chr10: 35549015-35549283. In some embodiments, the RET-RASGEF1A fusion nucleic acid molecule of the disclosure comprises or results from a breakpoint within chromosomal coordinates chr10: 43601756-43602031 and/or chr10: 43709383-43709498. In some embodiments, the RET-HSD17B7P2 fusion nucleic acid molecule of the disclosure comprises or results from a breakpoint within chromosomal coordinates chr10: 43609878-43609957 and/or chr10: 38667312-38667485. In some embodiments, the RET-RAD1 fusion nucleic acid molecule of the disclosure comprises or results from a breakpoint within chromosomal coordinates chr10: 43609716-43610036 and/or chr5: 34906366-34906711. In some embodiments, the RET-VSTM4 fusion nucleic acid molecule of the disclosure comprises or results from a breakpoint within chromosomal coordinates chr10: 43609237-43609437 and/or chr10: 50225719-50225999. In some embodiments, the ANKRD26-RET fusion nucleic acid molecule of the disclosure comprises or results from a breakpoint within chromosomal coordinates chr10: 27310264-27310361 and/or chr10: 43610383-43610480. In some embodiments, the CLIP1-RET fusion nucleic acid molecule of the disclosure comprises or results from a breakpoint within chromosomal coordinates chr12: 122805302-122805302 and/or chr10: 43611109-43611109. In some embodiments, the DLG5-RET fusion nucleic acid molecule of the disclosure comprises or results from a breakpoint within chromosomal coordinates chr10: 79582000-79582000 and/or chr10: 43611570-43611570. In some embodiments, the DLG5-RET fusion nucleic acid molecule of the disclosure comprises or results from a breakpoint within chromosomal coordinates chr10: 79559942-79559942 and/or chr10: 43607485-43607485. In some embodiments, the ERC1-RET fusion nucleic acid molecule of the disclosure comprises or results from a breakpoint within chromosomal coordinates chr12: 1346557-1346557 and/or chr10: 43610879-43610879. In some embodiments, the ERC1-RET fusion nucleic acid molecule of the disclosure comprises or results from a breakpoint within chromosomal coordinates chr12: 1290627-1290627 and/or chr10: 43611066-43611066. In some embodiments, the ERC1-RET fusion nucleic acid molecule of the disclosure comprises or results from a breakpoint within chromosomal coordinates chr12: 1350447-1350447 and/or chr10: 43609823-43609823. In some embodiments, the FRMD4A-RET fusion nucleic acid molecule of the disclosure comprises or results from a breakpoint within chromosomal coordinates chr10: 13758406-13758406 and/or chr10: 43610354-43610354. In some embodiments, the KIAA1468-RET fusion nucleic acid molecule of the disclosure comprises or results from a breakpoint within chromosomal coordinates chr18: 59908435-59908435 and/or chr10: 43611083-43611083. In some embodiments, the NCOA4-RET fusion nucleic acid molecule of the disclosure comprises or results from a breakpoint within chromosomal coordinates chr10: 51585637-51585735 and/or chr10: 43610257-43610403. In some embodiments, the PARD3-RET fusion nucleic acid molecule of the disclosure comprises or results from a breakpoint within chromosomal coordinates chr10: 34926445-34926445 and/or chr10: 43607138-43607138. In some embodiments, the PARD3-RET fusion nucleic acid molecule of the disclosure comprises or results from a breakpoint within chromosomal coordinates chr10: 34755707-34755707 and/or chr10: 43612003-43612003. In some embodiments, the PRKAR1A-RET fusion nucleic acid molecule of the disclosure comprises or results from a breakpoint within chromosomal coordinates chr17: 66523509-66523607 and/or chr10: 43611401-43611502. In some embodiments, the PRKG1-RET fusion nucleic acid molecule of the disclosure comprises or results from a breakpoint within chromosomal coordinates chr10: 53889856-53889954 and/or chr10: 43600288-43600402. In some embodiments, the PRKG1-RET fusion nucleic acid molecule of the disclosure comprises or results from a breakpoint within chromosomal coordinates chr 10:53789188-53789284 and/or chr10: 43620162-43620329. In some embodiments, the RUFY2-RET fusion nucleic acid molecule of the disclosure comprises or results from a breakpoint within chromosomal coordinates chr10: 70143736-70143833 and/or chr10: 43611405-43611519. In some embodiments, the SNRNP70-RET fusion nucleic acid molecule of the disclosure comprises or results from a breakpoint within chromosomal coordinates chr19: 49602640-49602640 and/or chr10: 43611323-43611323. In some embodiments, the SQSTM1-RET fusion nucleic acid molecule of the disclosure comprises or results from a breakpoint within chromosomal coordinates chr5: 179254189-179254286 and/or chr10: 43609433-43609563. In some embodiments, the TNIP1-RET fusion nucleic acid molecule of the disclosure comprises or results from a breakpoint within chromosomal coordinates chr5: 150416127-150416127 and/or chr10: 43611669-43611669. In some embodiments, the TNIP1-RET fusion nucleic acid molecule of the disclosure comprises or results from a breakpoint within chromosomal coordinates chr5: 150414253-150414351 and/or chr10: 43611007-43611109. In some embodiments, the TNIP1-RET fusion nucleic acid molecule of the disclosure comprises or results from a breakpoint within chromosomal coordinates chr5: 150417592-150417592 and/or chr10: 43611428-43611428. In some embodiments, the TRIM27-RET fusion nucleic acid molecule of the disclosure comprises or results from a breakpoint within chromosomal coordinates chr6: 28881702-28881890 and/or chr10: 43610709-43610878. In some embodiments, the TRIM33-RET fusion nucleic acid molecule of the disclosure comprises or results from a breakpoint within chromosomal coordinates chr1: 114965871-114965970 and/or chr10: 43611514-43611626. In some embodiments, the CCDC6-RET fusion nucleic acid molecule of the disclosure comprises or results from a breakpoint within chromosomal coordinates chr10: 61585545-61585642 and/or chr10: 43609686-43609783. In some embodiments, the TRIM24-RET fusion nucleic acid molecule of the disclosure comprises or results from a breakpoint within chromosomal coordinates chr7: 138267911-138267911 and/or chr10: 43611642-43611642. In some embodiments, the FGFR1OP-RET fusion nucleic acid molecule of the disclosure comprises or results from a breakpoint within chromosomal coordinates chr6: 167424630-167424630 and/or chr 10:43611296-43611296. In some embodiments, the GAS2-RET fusion nucleic acid molecule of the disclosure comprises or results from a breakpoint within chromosomal coordinates chr11: 22812170-22812170 and/or chr10: 43600385-43600385. In some embodiments, the HOOK1-RET fusion nucleic acid molecule of the disclosure comprises or results from a breakpoint within chromosomal coordinates chr1: 60334563-60334563 and/or chr10: 43610286-43610286. In some embodiments, the LMNA-RET fusion nucleic acid molecule of the disclosure comprises or results from a breakpoint within chromosomal coordinates chr1: 156102332-156102332 and/or chr10: 43611407-43611407. In some embodiments, the LMNA-RET fusion nucleic acid molecule of the disclosure comprises or results from a breakpoint within chromosomal coordinates chr1: 156102293-156102386 and/or chr10: 43611365-43611462. In some embodiments, the MLPH-RET fusion nucleic acid molecule of the disclosure comprises or results from a breakpoint within chromosomal coordinates chr2: 238445254-238445254 and/or chr10: 43610453-43610453. In some embodiments, the MYH9-RET fusion nucleic acid molecule of the disclosure comprises or results from a breakpoint within chromosomal coordinates chr22: 36683797-36683797 and/or chr10: 43611848-43611848. In some embodiments, the PCDH15-RET fusion nucleic acid molecule of the disclosure comprises or results from a breakpoint within chromosomal coordinates chr10: 56509282-56509282 and/or chr10: 43601819-43601819. In some embodiments, the PIBF1-RET fusion nucleic acid molecule of the disclosure comprises or results from a breakpoint within chromosomal coordinates chr13: 73550847-73550847 and/or chr10: 43611532-43611532. In some embodiments, the PIBF1-RET fusion nucleic acid molecule of the disclosure comprises or results from a breakpoint within chromosomal coordinates chr13: 73548007-73548007 and/or chr10: 43611167-43611167. In some embodiments, the SLC12A2-RET fusion nucleic acid molecule of the disclosure comprises or results from a breakpoint within chromosomal coordinates chr5: 127496774-127496774 and/or chr10: 43611855-43611855. In some embodiments, the ZNF485-RET fusion nucleic acid molecule of the disclosure comprises or results from a breakpoint within chromosomal coordinates chr10: 44109385-44109456 and/or chr10: 43611964-43612093. In some embodiments, the ZNF485-RET fusion nucleic acid molecule of the disclosure comprises or results from a breakpoint within chromosomal coordinates chr10: 44109396-44109396 and/or chr10: 43608521-43608521.

In some embodiments of any of the RET fusion nucleic acid molecules provided herein, the chromosomal coordinates corresponding to any of the breakpoints described herein correspond to Homo sapiens (human) genome assembly GRCh37 (hg19).

In some embodiments, a RET fusion nucleic acid molecule of the disclosure comprises or results from a fusion between a 5′ exon, or a portion thereof, and the corresponding 3′ exon, or a portion thereof, as indicated in Table 6.

TABLE 6
Exons fused in exemplary RET fusion nucleic acid molecules.

| Fusion Nucleic | | |
|---|---|---|
| Acid Molecule | 5′ Exon | 3′ Exon |
| GOLGB1 - RET | GOLGB1 exon 10 | RET exon 12 |
| GP2 - RET | GP2 exon 4 | RET exon11 |
| PPP2R5A - RET | PPP2R5A exon 1 | RET exon 20 |
| GRM7 - RET | GRM7 exon 9 | RET exon 3 |
| MYH14 - RET | MYH14 exon 37 | RET exon 12 |
| MGEA5 - RET | MGEA5 exon 12 | RET exon 12 |
| SAMD4A - RET | SAMD4A exon 2 | RET exon11 |
| ACPP - RET | ACPP exon 10 | RET exon11 |
| BMS1 - RET | BMS1 exon 13 | RET exon 10 |
| CEP135 - RET | CEP135 exon 11 | RET exon 12 |
| EEA1 - RET | EEA1 exon 13 | RET exon 12 |
| CSGALNACT2 - RET | CSGALNACT2 exon 6 | RET exon 12 |
| KIAA1217 - RET | KIAA1217 exon 11 | RET exon8 |
| SORBS1 - RET | SORBS1 exon 20 | RET exon7 |
| SORBS1 - RET | SORBS1 exon 22 | RET exon11 |
| MPRIP - RET | MPRIP exon 22 | RET exon 12 |
| TFG - RET | TFG exon 5 | RET exon 11 |
| SPECC1L - RET | SPECC1L exon 10 | RET exon 12 |
| REEP3 - RET | REEP3 exon 5 | RET exon 12 |
| RRBP1 - RET | RRBP1 exon 22 | RET exon 12 |
| ETV6 - RET | ETV6 exon 6 | RET exon 12 |
| TAF3 - RET | TAF3 exon 3 | RET exon 12 |
| PCM1 - RET | PCM1 exon 29 | RET exon 12 |
| PCM1 - RET | PCM1 exon 29 | RET exon 12 |
| TNIP2 - RET | TNIP2 exon 5 | RET exon 12 |
| SATB1 - RET | SATB1 exon 7 | RET exon 12 |
| RET - ADCY1 | RET exon 11 | ADCY1 exon 12 |
| RET - ZNF248 | RET exon 11 | ZNF248 exon 4 |
| RET - AGBL4 | RET exon 12 | AGBL4 exon 3 |
| RET - LRMDA | RET exon 10 | LRMDA exon 6 |
| RET - ARHGAP19 | RET exon 11 | ARHGAP19 exon 7 |
| RET - CPEB3 | RET exon 11 | CPEB3 exon 8 |
| RET - DCC | RET exon 9 | DCC exon 8 |
| RET - ELMO1 | RET exon 11 | ELMO1 exon 14 |
| RET - WDFY4 | RET exon 15 | WDFY4 exon 40 |
| MKX - RET | MKX exon 5 | RET exon 12 |
| RBMS3 - RET | RBMS3 exon 6 | RET exon 12 |
| SGIP1 - RET | SGIP1 exon 1 | RET exon 12 |
| ZSWIM6 - RET | ZSWIM6 exon 1 | RET exon 12 |
| ALOX5 - RET | ALOX5 exon 2 | RET exon 12 |
| RET - NTRK2 | RET exon 10 | NTRK2 exon 20 |
| RET - OXR1 | RET exon 11 | OXR1 exon 4 |
| RET - CCBE1 | RET exon 8 | CCBE1 exon 3 |
| RET - NAALADL2 | RET exon 11 | NAALADL2 exon 13 |
| RAI14 - RET | RAI14 exon 15 | RET exon11 |
| ABI3BP - RET | ABI3BP exon 35 | RET exon 12 |
| LINC00379 - RET | LINC00379 exon 2 | RET exon 12 |
| OPTN - RET | OPTN exon 1 | RET exon11 |
| SH2D3A - RET | SH2D3A exon 1 | RET exon 12 |
| ZNF721 - RET | ZNF721 exon 1 | RET exon 12 |
| ADAMTS14 - RET | ADAMTS14 exon 4 | RET exon 12 |
| CSGALNACT2 - RET | CSGALNACT2 exon 1 | RET exon 12 |
| CPEB3 - RET | CPEB3 exon 10 | RET exon 12 |
| RET - CCNY | RET exon 11 | CCNY exon 4 |
| RET - RASGEF1A | RET exon 5 | RASGEF1A exon 2 |
| RET - HSD17B7P2 | RET exon 11 | HSD17B7P2 exon 8 |
| RET - RAD1 | RET exon 11 | RAD1 exon 6 |
| RET - VSTM4 | RET exon 10 | VSTM4 exon 8 |
| ANKRD26 - RET | ANKRD26 exon 29 | RET exon 12 |
| CLIP1 - RET | CLIP1 exon 16 | RET exon 12 |
| DLG5 - RET | DLG5 exon 14 | RET exon 12 |
| DLG5 - RET | DLG5 exon 27 | RET exon 8 |
| ERC1 - RET | ERC1 exon 12 | RET exon 12 |
| ERC1 - RET | ERC1 exon 8 | RET exon 12 |
| ERC1 - RET | ERC1 exon 12 | RET exon 11 |
| FRMD4A - RET | FRMD4A exon 12 | RET exon 12 |
| KIAA1468 - RET | KIAA1468 exon 10 | RET exon 12 |
| NCOA4 - RET | NCOA4 exon 8 | RET exon 12 |
| PARD3 - RET | PARD3 exon 2 | RET exon 8 |
| PARD3 - RET | PARD3 exon 4 | RET exon 12 |
| PRKAR1A - RET | PRKAR1A exon 7 | RET exon 12 |
| PRKG1 - RET | PRKG1 exon 7 | RET exon 4 |
| PRKG1 - RET | PRKG1 exon 5 | RET exon 18 |
| RUFY2 - RET | RUFY2 exon 9 | RET exon 12 |
| SNRNP70 - RET | SNRNP70 exon 6 | RET exon 12 |
| SQSTM1 - RET | SQSTM1 exon 5 | RET exon 11 |
| TNIP1 - RET | TNIP1 exon 13 | RET exon 12 |
| TNIP1 - RET | TNIP1 exon 15 | RET exon 12 |
| TNIP1 - RET | TNIP1 exon 12 | RET exon 12 |
| TRIM27 - RET | TRIM27 exon 3 | RET exon 12 |
| TRIM33 - RET | TRIM33 exon 10 | RET exon 12 |
| CCDC6 - RET | CCDC6 exon 3 | RET exon 11 |
| TRIM24 - RET | TRIM24 exon 17 | RET exon 12 |
| FGFR1OP-RET | FGFR1OP exon 6 | RET exon 12 |
| GAS2-RET | GAS2 exon 6 | RET exon 4 |
| HOOK1-RET | HOOK1 exon 20 | RET exon 12 |
| LMNA-RET | LMNA exon 2 | RET exon 12 |
| MLPH-RET | MLPH exon 9 | RET exon 12 |
| MYH9-RET | MYH9 exon 34 | RET exon 12 |
| PCDH15-RET | PCDH15 exon 1 | RET exon 5 |
| PIBF1-RET | PIBF1 exon 16 | RET exon 12 |
| SLC12A2-RET | SLC12A2 exon 16 | RET exon 12 |
| ZNF485-RET | ZNF485 exon 4 | RET exon 12 |
| ZNF485-RET | ZNF485 exon 4 | RET exon 10 |

In some embodiments, the GOLGB1-RET fusion nucleic acid molecule of the disclosure comprises or results from a fusion, in 5′ to 3′ direction, of GOLGB1 exon 10, or a portion thereof, fused to RET exon 12, or a portion thereof. In some embodiments, the GP2-RET fusion nucleic acid molecule of the disclosure comprises or results from a fusion, in 5′ to 3′ direction, of GP2 exon 4, or a portion thereof, fused to RET exon 11, or a portion thereof. In some embodiments, the PPP2R5A-RET fusion nucleic acid molecule of the disclosure comprises or results from a fusion, in 5′ to 3′ direction, of PPP2R5A exon 1, or a portion thereof, fused to RET exon 20, or a portion thereof. In some embodiments, the GRM7-RET fusion nucleic acid molecule of the disclosure comprises or results from a fusion, in 5′ to 3′ direction, of GRM7 exon 9, or a portion thereof, fused to RET exon 3, or a portion thereof. In some embodiments, the MYH14-RET fusion nucleic acid molecule of the disclosure comprises or results from a fusion, in 5′ to 3′ direction, of MYH14 exon 37, or a portion thereof, fused to RET exon 12, or a portion thereof. In some embodiments, the MGEA5-RET fusion nucleic acid molecule of the disclosure comprises or results from a fusion, in 5′ to 3′ direction, of MGEA5 exon 12, or a portion thereof, fused to RET exon 12, or a portion thereof. In some embodiments, the SAMD4A-RET fusion nucleic acid molecule of the disclosure comprises or results from a fusion, in 5′ to 3′ direction, of SAMD4A exon 2, or a portion thereof, fused to RET exon11, or a portion thereof. In some embodiments, the ACPP-RET fusion nucleic acid molecule of the disclosure comprises or results from a fusion, in 5′ to 3′ direction, of ACPP exon 10, or a portion thereof, fused to RET exon11, or a portion thereof. In some embodiments, the BMS1-RET fusion nucleic acid molecule of the disclosure comprises or results from a fusion, in 5′ to 3′ direction, of BMS1 exon 13, or a portion thereof, fused to RET exon 10, or a portion thereof. In some embodiments, the CEP135-RET fusion nucleic acid molecule of the disclosure comprises or results from a fusion, in 5′ to 3′ direction, of CEP135 exon 11, or a portion thereof, fused to RET exon 12, or a portion thereof. In some embodiments, the EEA1-RET fusion nucleic acid molecule of the disclosure comprises or results from a fusion, in 5′ to 3′ direction, of EEA1 exon 13, or a portion thereof, fused to RET exon 12, or a portion thereof. In some embodiments, the CSGALNACT2-RET fusion nucleic acid molecule of the disclosure comprises or results from a fusion, in 5′ to 3′ direction, of CSGALNACT2 exon 6, or a portion thereof, fused to RET exon 12, or a portion thereof. In some embodiments, the KIAA1217-RET fusion nucleic acid molecule of the disclosure comprises or results from a fusion, in 5′ to 3′ direction, of KIAA1217 exon 11, or a portion thereof, fused to RET exon8, or a portion thereof. In some embodiments, the SORBS1-RET fusion nucleic acid molecule of the disclosure comprises or results from a fusion, in 5′ to 3′ direction, of SORBS1 exon 20, or a portion thereof, fused to RET exon7, or a portion thereof. In some embodiments, the SORBS1-RET fusion nucleic acid molecule of the disclosure comprises or results from a fusion, in the 5′ to 3′ direction, of SORBS1 exon 22, or a portion thereof, fused to RET exon11, or a portion thereof. In some embodiments, the MPRIP-RET fusion nucleic acid molecule of the disclosure comprises or results from a fusion, in 5′ to 3′ direction, of MPRIP exon 22, or a portion thereof, fused to RET exon 12, or a portion thereof. In some embodiments, the TFG-RET fusion nucleic acid molecule of the disclosure comprises or results from a fusion, in 5′ to 3′ direction, of TFG exon 5, or a portion thereof, fused to RET exon 11, or a portion thereof. In some embodiments, the SPECC1L-RET fusion nucleic acid molecule of the disclosure comprises or results from a fusion, in the 5′ to 3′ direction, of SPECC1L exon 10, or a portion thereof, fused to RET exon 12, or a portion thereof. In some embodiments, the REEP3-RET fusion nucleic acid molecule of the disclosure comprises or results from a fusion, in 5′ to 3′ direction, of REEP3 exon 5, or a portion thereof, fused to RET exon 12, or a portion thereof. In some embodiments, the RRBP1-RET fusion nucleic acid molecule of the disclosure comprises or results from a fusion, in 5′ to 3′ direction, of RRBP1 exon 22, or a portion thereof, fused to RET exon 12, or a portion thereof. In some embodiments, the ETV6-RET fusion nucleic acid molecule of the disclosure comprises or results from a fusion, in 5′ to 3′ direction, of ETV6 exon 6, or a portion thereof, fused to RET exon 12, or a portion thereof. In some embodiments, the TAF3-RET fusion nucleic acid molecule of the disclosure comprises or results from a fusion, in 5′ to 3′ direction, of TAF3 exon 3, or a portion thereof, fused to RET exon 12, or a portion thereof. In some embodiments, the PCM1-RET fusion nucleic acid molecule of the disclosure comprises or results from a fusion, in 5′ to 3′ direction, of PCM1 exon 29, or a portion thereof, fused to RET exon 12, or a portion thereof. In some embodiments, the PCM1-RET fusion nucleic acid molecule of the disclosure comprises or results from a fusion, in 5′ to 3′ direction, of PCM1 exon 29, or a portion thereof, fused to RET exon 12, or a portion thereof. In some embodiments, the TNIP2-RET fusion nucleic acid molecule of the disclosure comprises or results from a fusion, in 5′ to 3′ direction, of TNIP2 exon 5, or a portion thereof, fused to RET exon 12, or a portion thereof. In some embodiments, the SATB1-RET fusion nucleic acid molecule of the disclosure comprises or results from a fusion, in 5′ to 3′ direction, of SATB1 exon 7, or a portion thereof, fused to RET exon 12, or a portion thereof. In some embodiments, the RET-ADCY1 fusion nucleic acid molecule of the disclosure comprises or results from a fusion, in 5′ to 3′ direction, of RET exon 11, or a portion thereof, fused to ADCY1 exon 12, or a portion thereof. In some embodiments, the RET-ZNF248 fusion nucleic acid molecule of the disclosure comprises or results from a fusion, in 5′ to 3′ direction, of RET exon 11, or a portion thereof, fused to ZNF248 exon 4, or a portion thereof. In some embodiments, the RET-AGBL4 fusion nucleic acid molecule of the disclosure comprises or results from a fusion, in 5′ to 3′ direction, of RET exon 12, or a portion thereof, fused to AGBL4 exon 3, or a portion thereof. In some embodiments, the RET-LRMDA fusion nucleic acid molecule of the disclosure comprises or results from a fusion, in 5′ to 3′ direction, of RET exon 10, or a portion thereof, fused to LRMDA exon 6, or a portion thereof. In some embodiments, the RET-ARHGAP19 fusion nucleic acid molecule of the disclosure comprises or results from a fusion, in 5′ to 3′ direction, of RET exon 11, or a portion thereof, fused to ARHGAP19 exon 7, or a portion thereof. In some embodiments, the RET-CPEB3 fusion nucleic acid molecule of the disclosure comprises or results from a fusion, in 5′ to 3′ direction, of RET exon 11, or a portion thereof, fused to CPEB3 exon 8, or a portion thereof. In some embodiments, the RET-DCC fusion nucleic acid molecule of the disclosure comprises or results from a fusion, in 5′ to 3″ direction, of RET exon 9, or a portion thereof, fused to DCC exon 8, or a portion thereof. In some embodiments, the RET-ELMO1 fusion nucleic acid molecule of the disclosure comprises or results from a fusion, in 5′ to 3′ direction, of RET exon 11, or a portion thereof, fused to ELMO1 exon 14, or a portion thereof. In some embodiments, the RET-WDFY4 fusion nucleic acid molecule of the disclosure comprises or results from a fusion, in 5′ to 3′ direction, of RET exon 15, or a portion thereof, fused to WDFY4 exon 40, or a portion thereof. In some embodiments, the MKX-RET fusion nucleic acid molecule of the disclosure comprises or results from a fusion, in 5′ to 3′ direction, of MKX exon 5, or a portion thereof, fused to RET exon 12, or a portion thereof. In some embodiments, the RBMS3-RET fusion nucleic acid molecule of the disclosure comprises or results from a fusion, in the 5′ to 3′ direction, of RBMS3 exon 6, or a portion thereof, fused to RET exon 12, or a portion thereof. In some embodiments, the SGIP1-RET fusion nucleic acid molecule of the disclosure comprises or results from a fusion, in 5′ to 3′ direction, of SGIP1 exon 1, or a portion thereof, fused to RET exon 12, or a portion thereof. In some embodiments, the ZSWIM6-RET fusion nucleic acid molecule of the disclosure comprises or results from a fusion, in 5′ to 3′ direction, of ZSWIM6 exon 1, or a portion thereof, fused to RET exon 12, or a portion thereof. In some embodiments, the ALOX5-RET fusion nucleic acid molecule of the disclosure comprises or results from a fusion, in 5′ to 3′ direction, of ALOX5 exon 2, or a portion thereof, fused to RET exon 12, or a portion thereof. In some embodiments, the RET-NTRK2 fusion nucleic acid molecule of the disclosure comprises or results from a fusion, in 5′ to 3′ direction, of RET exon 10, or a portion thereof, fused to NTRK2 exon 20, or a portion thereof. In some embodiments, the RET-OXR1 fusion nucleic acid molecule of the disclosure comprises or results from a fusion, in 5′ to 3′ direction, of RET exon 11, or a portion thereof, fused to OXR1 exon 4, or a portion thereof. In some embodiments, the RET-CCBE1 fusion nucleic acid molecule of the disclosure comprises or results from a fusion, in the 5′ to 3′ direction, of RET exon 8, or a portion thereof, fused to CCBE1 exon 3, or a portion thereof. In some embodiments, the RET-NAALADL2 fusion nucleic acid molecule of the disclosure comprises or results from a fusion, in 5′ to 3′ direction, of RET exon 11, or a portion thereof, fused to NAALADL2 exon 13, or a portion thereof. In some embodiments, the RAI14-RET fusion nucleic acid molecule of the disclosure comprises or results from a fusion, in 5′ to 3′ direction, of RAI14 exon 15, or a portion thereof, fused to RET exon11, or a portion thereof. In some embodiments, the ABI3BP-RET fusion nucleic acid molecule of the disclosure comprises or results from a fusion, in 5′ to 3′ direction, of ABI3BP exon 35, or a portion thereof, fused to RET exon 12, or a portion thereof. In some embodiments, the LINC00379-RET fusion nucleic acid molecule of the disclosure comprises or results from a fusion, in 5′ to 3′ direction, of LINC00379 exon 2, or a portion thereof, fused to RET exon 12, or a portion thereof. In some embodiments, the OPTN-RET fusion nucleic acid molecule of the disclosure comprises or results from a fusion, in 5′ to 3′ direction, of OPTN exon 1, or a portion thereof, fused to RET exon11, or a portion thereof. In some embodiments, the SH2D3A-RET fusion nucleic acid molecule of the disclosure comprises or results from a fusion, in 5′ to 3′ direction, of SH2D3A exon 1, or a portion thereof, fused to RET exon 12, or a portion thereof. In some embodiments, the ZNF721-RET fusion nucleic acid molecule of the disclosure comprises or results from a fusion, in 5′ to 3′ direction, of ZNF721 exon 1, or a portion thereof, fused to RET exon 12, or a portion thereof. In some embodiments, the ADAMTS14-RET fusion nucleic acid molecule of the disclosure comprises or results from a fusion, in 5′ to 3″ direction, of ADAMTS14 exon 4, or a portion thereof, fused to RET exon 12, or a portion thereof. In some embodiments, the CSGALNACT2-RET fusion nucleic acid molecule of the disclosure comprises or results from a fusion, in 5′ to 3′ direction, of CSGALNACT2 exon 1, or a portion thereof, fused to RET exon 12, or a portion thereof. In some embodiments, the CPEB3-RET fusion nucleic acid molecule of the disclosure comprises or results from a fusion, in 5′ to 3′ direction, of CPEB3 exon 10, or a portion thereof, fused to RET exon 12, or a portion thereof. In some embodiments, the RET-CCNY fusion nucleic acid molecule of the disclosure comprises or results from a fusion, in 5′ to 3′ direction, of RET exon 11, or a portion thereof, fused to CCNY exon 4, or a portion thereof. In some embodiments, the RET-RASGEF1A fusion nucleic acid molecule of the disclosure comprises or results from a fusion, in 5′ to 3′ direction, of RET exon 5, or a portion thereof, fused to RASGEF1A exon 2, or a portion thereof. In some embodiments, the RET-HSD17B7P2 fusion nucleic acid molecule of the disclosure comprises or results from a fusion, in the 5′ to 3′ direction, of RET exon 11, or a portion thereof, fused to HSD17B7P2 exon 8, or a portion thereof. In some embodiments, the RET-RAD1 fusion nucleic acid molecule of the disclosure comprises or results from a fusion, in 5′ to 3′ direction, of RET exon 11, or a portion thereof, fused to RAD1 exon 6, or a portion thereof. In some embodiments, the RET-VSTM4 fusion nucleic acid molecule of the disclosure comprises or results from a fusion, in 5′ to 3′ direction, of RET exon 10, or a portion thereof, fused to VSTM4 exon 8, or a portion thereof. In some embodiments, the ANKRD26-RET fusion nucleic acid molecule of the disclosure comprises or results from a fusion, in the 5′ to 3′ direction, of ANKRD26 exon 29, or a portion thereof, fused to RET exon 12, or a portion thereof. In some embodiments, the CLIP1-RET fusion nucleic acid molecule of the disclosure comprises or results from a fusion, in 5′ to 3′ direction, of CLIP1 exon 16, or a portion thereof, fused to RET exon 12, or a portion thereof. In some embodiments, the DLG5-RET fusion nucleic acid molecule of the disclosure comprises or results from a fusion, in 5′ to 3′ direction, of DLG5 exon 14, or a portion thereof, fused to RET exon 12, or a portion thereof. In some embodiments, the DLG5-RET fusion nucleic acid molecule of the disclosure comprises or results from a fusion, in 5′ to 3′ direction, of DLG5 exon 27, or a portion thereof, fused to RET exon 8, or a portion thereof. In some embodiments, the ERC1-RET fusion nucleic acid molecule of the disclosure comprises or results from a fusion, in 5′ to 3′ direction, of ERC1 exon 12, or a portion thereof, fused to RET exon 12, or a portion thereof. In some embodiments, the ERC1-RET fusion nucleic acid molecule of the disclosure comprises or results from a fusion, in 5′ to 3′ direction, of ERC1 exon 8, or a portion thereof, fused to RET exon 12, or a portion thereof. In some embodiments, the ERC1-RET fusion nucleic acid molecule of the disclosure comprises or results from a fusion, in 5′ to 3′ direction, of ERC1 exon 12, or a portion thereof, fused to RET exon 11, or a portion thereof. In some embodiments, the FRMD4A-RET fusion nucleic acid molecule of the disclosure comprises or results from a fusion, in 5′ to 3′ direction, of FRMD4A exon 12, or a portion thereof, fused to RET exon 12, or a portion thereof. In some embodiments, the KIAA1468-RET fusion nucleic acid molecule of the disclosure comprises or results from a fusion, in 5′ to 3′ direction, of KIAA1468 exon 10, or a portion thereof, fused to RET exon 12, or a portion thereof. In some embodiments, the NCOA4-RET fusion nucleic acid molecule of the disclosure comprises or results from a fusion, in 5′ to 3′ direction, of NCOA4 exon 8, or a portion thereof, fused to RET exon 12, or a portion thereof. In some embodiments, the PARD3-RET fusion nucleic acid molecule of the disclosure comprises or results from a fusion, in 5′ to 3′ direction, of PARD3 exon 2, or a portion thereof, fused to RET exon 8, or a portion thereof. In some embodiments, the PARD3-RET fusion nucleic acid molecule of the disclosure comprises or results from a fusion, in 5′ to 3′ direction, of PARD3 exon 4, or a portion thereof, fused to RET exon 12, or a portion thereof. In some embodiments, the PRKAR1A-RET fusion nucleic acid molecule of the disclosure comprises or results from a fusion, in 5′ to 3′ direction, of PRKAR1A exon 7, or a portion thereof, fused to RET exon 12, or a portion thereof. In some embodiments, the PRKG1-RET fusion nucleic acid molecule of the disclosure comprises or results from a fusion, in 5′ to 3′ direction, of PRKG1 exon 7, or a portion thereof, fused to RET exon 4, or a portion thereof. In some embodiments, the PRKG1-RET fusion nucleic acid molecule of the disclosure comprises or results from a fusion, in 5′ to 3′ direction, of PRKG1 exon 5, or a portion thereof, fused to RET exon 18, or a portion thereof. In some embodiments, the RUFY2-RET fusion nucleic acid molecule of the disclosure comprises or results from a fusion, in 5′ to 3′ direction, of RUFY2 exon 9, or a portion thereof, fused to RET exon 12, or a portion thereof. In some embodiments, the SNRNP70-RET fusion nucleic acid molecule of the disclosure comprises or results from a fusion, in 5′ to 3′ direction, of SNRNP70 exon 6, or a portion thereof, fused to RET exon 12, or a portion thereof. In some embodiments, the SQSTM1-RET fusion nucleic acid molecule of the disclosure comprises or results from a fusion, in 5′ to 3′ direction, of SQSTM1 exon 5, or a portion thereof, fused to RET exon 11, or a portion thereof. In some embodiments, the TNIP1-RET fusion nucleic acid molecule of the disclosure comprises or results from a fusion, in 5′ to 3′ direction, of TNIP1 exon 13, or a portion thereof, fused to RET exon 12, or a portion thereof. In some embodiments, the TNIP1-RET fusion nucleic acid molecule of the disclosure comprises or results from a fusion, in 5′ to 3′ direction, of TNIP1 exon 15, or a portion thereof, fused to RET exon 12, or a portion thereof. In some embodiments, the TNIP1-RET fusion nucleic acid molecule of the disclosure comprises or results from a fusion, in 5′ to 3′ direction, of TNIP1 exon 12, or a portion thereof, fused to RET exon 12, or a portion thereof. In some embodiments, the TRIM27-RET fusion nucleic acid molecule of the disclosure comprises or results from a fusion, in 5′ to 3′ direction, of TRIM27 exon 3, or a portion thereof, fused to RET exon 12, or a portion thereof. In some embodiments, the TRIM33-RET fusion nucleic acid molecule of the disclosure comprises or results from a fusion, in 5′ to 3′ direction, of TRIM33 exon 10, or a portion thereof, fused to RET exon 12, or a portion thereof. In some embodiments, the CCDC6-RET fusion nucleic acid molecule of the disclosure comprises or results from a fusion, in 5′ to 3′ direction, of CCDC6 exon 3, or a portion thereof, fused to RET exon 11, or a portion thereof. In some embodiments, the TRIM24-RET fusion nucleic acid molecule of the disclosure comprises or results from a fusion, in 5′ to 3′ direction, of TRIM24 exon 17, or a portion thereof, fused to RET exon 12, or a portion thereof. In some embodiments, the FGFR1OP-RET fusion nucleic acid molecule of the disclosure comprises or results from a fusion, in 5′ to 3′ direction, of FGFR1OP exon 6, or a portion thereof, fused to RET exon 12, or a portion thereof. In some embodiments, the GAS2-RET fusion nucleic acid molecule of the disclosure comprises or results from a fusion, in 5′ to 3′ direction, of GAS2 exon 6, or a portion thereof, fused to RET exon 4, or a portion thereof. In some embodiments, the HOOK1-RET fusion nucleic acid molecule of the disclosure comprises or results from a fusion, in 5′ to 3′ direction, of HOOK1 exon 20, or a portion thereof, fused to RET exon 12, or a portion thereof. In some embodiments, the LMNA-RET fusion nucleic acid molecule of the disclosure comprises or results from a fusion, in 5′ to 3′ direction, of LMNA exon 2, or a portion thereof, fused to RET exon 12, or a portion thereof. In some embodiments, the MLPH-RET fusion nucleic acid molecule of the disclosure comprises or results from a fusion, in 5′ to 3′ direction, of MLPH exon 9, or a portion thereof, fused to RET exon 12, or a portion thereof. In some embodiments, the MYH9-RET fusion nucleic acid molecule of the disclosure comprises or results from a fusion, in 5′ to 3′ direction, of MYH9 exon 34, or a portion thereof, fused to RET exon 12, or a portion thereof. In some embodiments, the PCDH15-RET fusion nucleic acid molecule of the disclosure comprises or results from a fusion, in 5′ to 3′ direction, of PCDH15 exon 1, or a portion thereof, fused to RET exon 5, or a portion thereof. In some embodiments, the PIBF1-RET fusion nucleic acid molecule of the disclosure comprises or results from a fusion, in 5′ to 3′ direction, of PIBF1 exon 16, or a portion thereof, fused to RET exon 12, or a portion thereof. In some embodiments, the SLC12A2-RET fusion nucleic acid molecule of the disclosure comprises or results from a fusion, in 5′ to 3′ direction, of SLC12A2 exon 16, or a portion thereof, fused to RET exon 12, or a portion thereof. In some embodiments, the ZNF485-RET fusion nucleic acid molecule of the disclosure comprises or results from a fusion, in 5′ to 3′ direction, of ZNF485 exon 4, or a portion thereof, fused to RET exon 12, or a portion thereof. In some embodiments, the ZNF485-RET fusion nucleic acid molecule of the disclosure comprises or results from a fusion, in 5′ to 3′ direction, of ZNF485 exon 4, or a portion thereof, fused to RET exon 10, or a portion thereof.

In some embodiments, a RET fusion nucleic acid molecule of the disclosure comprises, in the 5′ to 3′ direction, the corresponding exons or portions thereof as listed in Table 7.

TABLE 7
Exons in exemplary RET fusion nucleic acid molecules.

| RET Fusion Nucleic Acid | |
|---|---|
| Molecule | Exons fused, in the 5′ to 3′ direction |
| GOLGB1 - RET | Exons 1-9, and exon 10 or a portion thereof, of GOLGB1 fused |
| | to exon 12 or a portion thereof, and exons 13-19, of RET |
| GP2 - RET | Exons 1-3, and exon 4 or a portion thereof, of GP2 fused to |
| | exon 11 or a portion thereof, and exons 12-19, of RET |
| PPP2R5A - RET | Exon 1 or a portion thereof, of PPP2R5A fused to exon 20 or a |
| | portion thereof, of RET |
| GRM7 - RET | Exons 1-8, and exon 9 or a portion thereof, of GRM7 fused to |
| | exon 3 or a portion thereof, and exons 4-19, of RET |
| MYH14 - RET | Exons 1-36, and exon 37 or a portion thereof, of MYH14 fused |
| | to exon 12 or a portion thereof, and exons 13-19, of RET |
| MGEA5 - RET | Exons 1-11, and exon 12 or a portion thereof, of MGEA5 fused |
| | to exon 12 or a portion thereof, and exons 13-19, of RET |
| SAMD4A - RET | Exon 1, and exon 2 or a portion thereof, of SAMD4A fused to |
| | exon 11 or a portion thereof, and exons 12-19, of RET |
| ACPP - RET | Exons 1-9, and exon 10 or a portion thereof, of ACPP fused to |
| | exon 11 or a portion thereof, and exons 12-19, of RET |
| BMS1 - RET | Exons 1-12, and exon 13 or a portion thereof, of BMS1 fused to |
| | exon 10 or a portion thereof, and exons 11-19, of RET |
| CEP135 - RET | Exons 1-10, and exon 11 or a portion thereof, of CEP135 fused |
| | to exon 12 or a portion thereof, and exons 13-19, of RET |
| EEA1 - RET | Exons 1-12, and exon 13 or a portion thereof, of EEA1 fused to |
| | exon 12 or a portion thereof, and exons 13-19, of RET |
| CSGALNACT2 - RET | Exons 1-5, and exon 6 or a portion thereof, of CSGALNACT2 |
| | fused to exon 12 or a portion thereof, and exons 13-19, of RET |
| KIAA1217 - RET | Exons 1-10, and exon 11 or a portion thereof, of KIAA1217 |
| | fused to exon 8 or a portion thereof, and exons 9-19, of RET |
| SORBS1 - RET | Exons 1-19, and exon 20 or a portion thereof, of SORBS1 fused |
| | to exon 7 or a portion thereof, and exons 8-19, of RET |
| SORBS1 - RET | Exons 1-21, and exon 22 or a portion thereof, of SORBS1 fused |
| | to exon 11 or a portion thereof, and exons 12-19, of RET |
| MPRIP - RET | Exons 1-21, and exon 22 or a portion thereof, of MPRIP fused |
| | to exon 12 or a portion thereof, and exons 13-19, of RET |
| TFG - RET | Exons 1-4, and exon 5 or a portion thereof, of TFG fused to |
| | exon 11 or a portion thereof, and exons 12-19, of RET |
| SPECC1L - RET | Exons 1-9, and exon 10 or a portion thereof, of SPECC1L fused |
| | to exon 12 or a portion thereof, and exons 13-19, of RET |
| REEP3 - RET | Exons 1-4, and exon 5 or a portion thereof, of REEP3 fused to |
| | exon 12 or a portion thereof, and exons 13-19, of RET |
| RRBP1 - RET | Exons 1-21, and exon 22 or a portion thereof, of RRBP1 fused |
| | to exon 12 or a portion thereof, and exons 13-19, of RET |
| ETV6 - RET | Exons 1-5, and exon 6 or a portion thereof, of ETV6 fused to |
| | exon 12 or a portion thereof, and exons 13-19, of RET |
| TAF3 - RET | Exons 1-2, and exon 3 or a portion thereof, of TAF3 fused to |
| | exon 12 or a portion thereof, and exons 13-19, of RET |
| PCM1 - RET | Exons 1-28, and exon 29 or a portion thereof, of PCM1 fused to |
| | exon 12 or a portion thereof, and exons 13-19, of RET |
| TNIP2 - RET | Exons 1-4, and exon 5 or a portion thereof, of TNIP2 fused to |
| | exon 12 or a portion thereof, and exons 13-19, of RET |
| SATB1 - RET | Exons 1-6, and exon 7 or a portion thereof, of SATB1 fused to |
| | exon 12 or a portion thereof, and exons 13-19, of RET |
| RET - ADCY1 | Exons 1-10, and exon 11 or a portion thereof, of RET fused to |
| | exon 12 or a portion thereof, and exons 13-20, of ADCY1 |
| RET - ZNF248 | Exons 1-10, and exon 11 or a portion thereof, of RET fused to |
| | exon 4 or a portion thereof, and exons 5-6, of ZNF248 |
| RET - AGBL4 | Exons 1-11, and exon 12 or a portion thereof, of RET fused to |
| | exon 3 or a portion thereof, and exons 4-14, of AGBL4 |
| RET - LRMDA | Exons 1-9, and exon 10 or a portion thereof, of RET fused to |
| | exon 6 or a portion thereof, of LRMDA |
| RET - ARHGAP19 | Exons 1-10, and exon 11 or a portion thereof, of RET fused to |
| | exon 7 or a portion thereof, and exons 8-12, of ARHGAP19 |
| RET - CPEB3 | Exons 1-10, and exon 11 or a portion thereof, of RET fused to |
| | exon 8 or a portion thereof, and exons 9-10, of CPEB3 |
| RET - DCC | Exons 1-8, and exon 9 or a portion thereof, of RET fused to |
| | exon 8 or a portion thereof, and exons 9-29, of DCC |
| RET - ELMO1 | Exons 1-10, and exon 11 or a portion thereof, of RET fused to |
| | exon 14 or a portion thereof, and exons 15-22, of ELMO1 |
| RET - WDFY4 | Exons 1-14, and exon 15 or a portion thereof, of RET fused to |
| | exon 40 or a portion thereof, and exons 41-62, of WDFY4 |
| MKX - RET | Exons 1-4, and exon 5 or a portion thereof, of MKX fused to |
| | exon 12 or a portion thereof, and exons 13-19, of RET |
| RBMS3 - RET | Exons 1-5, and exon 6 or a portion thereof, of RBMS3 fused to |
| | exon 12 or a portion thereof, and exons 13-19, of RET |
| SGIP1 - RET | Exon 1 or a portion thereof, of SGIP1 fused to exon 12 or a |
| | portion thereof, and exons 13-19, of RET |
| ZSWIM6 - RET | Exon 1 or a portion thereof, of ZSWIM6 fused to exon 12 or a |
| | portion thereof, and exons 13-19, of RET |
| ALOX5 - RET | Exon 1, and exon 2 or a portion thereof, of ALOX5 fused to |
| | exon 12 or a portion thereof, and exons 13-19, of RET |
| RET - NTRK2 | Exons 1-9, and exon 10 or a portion thereof, of RET fused to |
| | exon 20 or a portion thereof, and exon 21, of NTRK2 |
| RET - OXR1 | Exons 1-10, and exon 11 or a portion thereof, of RET fused to |
| | exon 4 or a portion thereof, and exons 5-16, of OXR1 |
| RET - CCBE1 | Exons 1-7, and exon 8 or a portion thereof, of RET fused to |
| | exon 3 or a portion thereof, and exons 4-11, of CCBE1 |
| RET - NAALADL2 | Exons 1-10, and exon 11 or a portion thereof, of RET fused to |
| | exon 13 or a portion thereof, and exon 14, of NAALADL2 |
| RAI14 - RET | Exons 1-14, and exon 15 or a portion thereof, of RAI14 fused to |
| | exon 11 or a portion thereof, and exons 12-19, of RET |
| ABI3BP - RET | Exons 1-34, and exon 35 or a portion thereof, of ABI3BP fused |
| | to exon 12 or a portion thereof, and exons 13-19, of RET |
| LINC00379 - RET | Exon 1, and exon 2 or a portion thereof, of LINC00379 fused to |
| | exon 12 or a portion thereof, and exons 13-19, of RET |
| OPTN - RET | Exon lor a portion thereof, of OPTN fused to exon 11 or a |
| | portion thereof, and exons 12-19, of RET |
| SH2D3A - RET | Exon 1 or a portion thereof, of SH2D3A fused to exon 12 or a |
| | portion thereof, and exons 13-19, of RET |
| ZNF721 - RET | Exon 1 or a portion thereof, of ZNF721 fused to exon 12 or a |
| | portion thereof, and exons 13-19, of RET |
| ADAMTS14 - RET | Exons 1-3, and exon 4 or a portion thereof, of ADAMTS14 |
| | fused to exon 12 or a portion thereof, and exons 13-19, of RET |
| CSGALNACT2 - RET | Exon 1 or a portion thereof, of CSGALNACT2 fused to exon |
| | 12 or a portion thereof, and exons 13-19, of RET |
| CPEB3 - RET | Exons 1-9, and exon 10 or a portion thereof, of CPEB3 fused to |
| | exon 12 or a portion thereof, and exons 13-19, of RET |
| RET - CCNY | Exons 1-10, and exon 11 or a portion thereof, of RET fused to |
| | exon 4 or a portion thereof, and exons 5-12, of CCNY |
| RET - RASGEF1A | Exons 1-4, and exon 5 or a portion thereof, of RET fused to |
| | exon 2 or a portion thereof, and exons 3-13, of RASGEF1A |
| RET - HSD17B7P2 | Exons 1-10, and exon 11 or a portion thereof, of RET fused to |
| | exon 8 or a portion thereof, of HSD17B7P2 |
| RET - RAD1 | Exons 1-10, and exon 11 or a portion thereof, of RET fused to |
| | exon 6 or a portion thereof, of RAD1 |
| RET - VSTM4 | Exons 1-9, and exon 10 or a portion thereof, of RET fused to |
| | exon 8 or a portion thereof, of VSTM4 |
| ANKRD26 - RET | Exons 1-28, and exon 29 or a portion thereof, of ANKRD26 |
| | fused to exon 12 or a portion thereof, and exons 13-19, of RET |
| CLIP1 - RET | Exons 1-15, and exon 16 or a portion thereof, of CLIP1 fused to |
| | exon 12 or a portion thereof, and exons 13-19, of RET |
| DLG5 - RET | Exons 1-13, and exon 14 or a portion thereof, of DLG5 fused to |
| | exon 12 or a portion thereof, and exons 13-19, of RET |
| DLG5 - RET | Exons 1-26, and exon 27 or a portion thereof, of DLG5 fused to |
| | exon 8 or a portion thereof, and exons 9-19, of RET |
| ERC1 - RET | Exons 1-11, and exon 12 or a portion thereof, of ERC1 fused to |
| | exon 12 or a portion thereof, and exons 13-19, of RET |
| ERC1 - RET | Exons 1-7, and exon 8 or a portion thereof, of ERC1 fused to |
| | exon 12 or a portion thereof, and exons 13-19, of RET |
| ERC1 - RET | Exons 1-11, and exon 12 or a portion thereof, of ERC1 fused to |
| | exon 11 or a portion thereof, and exons 12-19, of RET |
| FRMD4A - RET | Exons 1-11, and exon 12 or a portion thereof, of FRMD4A |
| | fused to exon 12 or a portion thereof, and exons 13-19, of RET |
| KIAA1468 - RET | Exons 1-9, and exon 10 or a portion thereof, of KIAA1468 |
| | fused to exon 12 or a portion thereof, and exons 13-19, of RET |
| NCOA4 - RET | Exons 1-7, and exon 8 or a portion thereof, of NCOA4 fused to |
| | exon 12 or a portion thereof, and exons 13-19, of RET |
| PARD3 - RET | Exon 1, and exon 2 or a portion thereof, of PARD3 fused to |
| | exon 8 or a portion thereof, and exons 9-19, of RET |
| PARD3 - RET | Exons 1-3, and exon 4 or a portion thereof, of PARD3 fused to |
| | exon 12 or a portion thereof, and exons 13-19, of RET |
| PRKAR1A - RET | Exons 1-6, and exon 7 or a portion thereof, of PRKAR1A fused |
| | to exon 12 or a portion thereof, and exons 13-19, of RET |
| PRKG1 - RET | Exons 1-6, and exon 7 or a portion thereof, of PRKG1 fused to |
| | exon 4 or a portion thereof, and exons 5-19, of RET |
| PRKG1 - RET | Exons 1-4, and exon 5 or a portion thereof, of PRKG1 fused to |
| | exon 18 or a portion thereof, and exon 19, of RET |
| RUFY2 - RET | Exons 1-8, and exon 9 or a portion thereof, of RUFY2 fused to |
| | exon 12 or a portion thereof, and exons 13-19, of RET |
| SNRNP70 - RET | Exons 1-5, and exon 6 or a portion thereof, of SNRNP70 fused |
| | to exon 12 or a portion thereof, and exons 13-19, of RET |
| SQSTM1 - RET | Exons 1-4, and exon 5 or a portion thereof, of SQSTM1 fused |
| | to exon 11 or a portion thereof, and exons 12-19, of RET |
| TNIP1 - RET | Exons 1-12, and exon 13 or a portion thereof, of TNIP1 fused to |
| | exon 12 or a portion thereof, and exons 13-19, of RET |
| TNIP1 - RET | Exons 1-14, and exon 15 or a portion thereof, of TNIP1 fused to |
| | exon 12 or a portion thereof, and exons 13-19, of RET |
| TNIP1 - RET | Exons 1-11, and exon 12 or a portion thereof, of TNIP1 fused to |
| | exon 12 or a portion thereof, and exons 13-19, of RET |
| TRIM27 - RET | Exons 1-2, and exon 3 or a portion thereof, of TRIM27 fused to |
| | exon 12 or a portion thereof, and exons 13-19, of RET |
| TRIM33 - RET | Exons 1-9, and exon 10 or a portion thereof, of TRIM33 fused |
| | to exon 12 or a portion thereof, and exons 13-19, of RET |
| CCDC6 - RET | Exons 1-2, and exon 3 or a portion thereof, of CCDC6 fused to |
| | exon 11 or a portion thereof, and exons 12-19, of RET |
| TRIM24 - RET | Exons 1-16, and exon 17 or a portion thereof, of TRIM24 fused |
| | to exon 12 or a portion thereof, and exons 13-19, of RET |
| FGFR1OP-RET | Exons 1-5 and exon 6 or a portion thereof, of FGFR1OP fused |
| | to exon 12 or a portion thereof, and exons 13-19, of RET |
| GAS2-RET | Exons 1-5, and exon 6 or a portion thereof, of GAS2 fused to |
| | exon 4 or a portion thereof, and exons 5-19, of RET |
| HOOK1-RET | Exons 1-19, and exon 20 or a portion thereof, of HOOK1 fused |
| | to exon 12 or portion thereof, and exons 13-19, of RET |
| LMNA-RET | Exon 1, and exon 2 or a portion thereof, of LMNA fused to |
| | exon 12 or a portion thereof, and exons 13-19, of RET |
| MLPH-RET | Exons 1-8, and exon 9 or a portion thereof, of MLPH fused to |
| | exon 12 or a portion thereof, and exons 13-19, of RET |
| MYH9-RET | Exons 1-33, and exon 34 or a portion thereof, of MYH9 fused |
| | to exon 12 or a portion thereof, and exons 13-19, of RET |
| PCDH15-RET | Exon 1 or a portion thereof, of PCDH15 fused to exon 5 or a |
| | portion thereof, and exons 6-19, of RET |
| PIBF1-RET | Exons 1-15, and exon 16 or a portion thereof, of PIBF1 fused to |
| | exon 12 or a portion thereof, and exons 13-19, of RET |
| SLC12A2-RET | Exons 1-15, and exon 16 or a portion thereof, of SLC12A2 |
| | fused to exon 12 or a portion thereof, and exons 13-19, of RET |
| ZNF485-RET | Exons 1-3, and exon 4 or a portion thereof, of ZNF485 fused to |
| | exon 12 or a portion thereof, and exons 13-19 of RET |
| ZNF485-RET | Exons 1-3, and exon 4 or a portion thereof, of ZNF485 fused to |
| | exon 10 or a portion thereof, and exons 11-19, of RET |

In some embodiments, the GOLGB1-RET fusion nucleic acid molecule of the disclosure comprises, in 5′ to 3′ direction, exons 1-9, and exon 10 or a portion thereof, of GOLGB1 fused to exon 12 or a portion thereof, and exons 13-19, of RET. In some embodiments, the GP2-RET fusion nucleic acid molecule of the disclosure comprises, in 5′ to 3′ direction, exons 1-3, and exon 4 or a portion thereof, of GP2 fused to exon 11 or a portion thereof, and exons 12-19, of RET. In some embodiments, the PPP2R5A-RET fusion nucleic acid molecule of the disclosure comprises, in the 5″ to 3′ direction, exon 1 or a portion thereof, of PPP2R5A fused to exon 20 or a portion thereof, of RET. In some embodiments, the GRM7-RET fusion nucleic acid molecule of the disclosure comprises, in 5′ to 3′ direction, exons 1-8, and exon 9 or a portion thereof, of GRM7 fused to exon 3 or a portion thereof, and exons 4-19, of RET. In some embodiments, the MYH14-RET fusion nucleic acid molecule of the disclosure comprises, in 5′ to 3′ direction, exons 1-36, and exon 37 or a portion thereof, of MYH14 fused to exon 12 or a portion thereof, and exons 13-19, of RET. In some embodiments, the MGEA5-RET fusion nucleic acid molecule of the disclosure comprises, in 5′ to 3′ direction, exons 1-11, and exon 12 or a portion thereof, of MGEA5 fused to exon 12 or a portion thereof, and exons 13-19, of RET. In some embodiments, the SAMD4A-RET fusion nucleic acid molecule of the disclosure comprises, in 5′ to 3′ direction, exon 1, and exon 2 or a portion thereof, of SAMD4A fused to exon 11 or a portion thereof, and exons 12-19, of RET. In some embodiments, the ACPP-RET fusion nucleic acid molecule of the disclosure comprises, in 5′ to 3′ direction, exons 1-9, and exon 10 or a portion thereof, of ACPP fused to exon 11 or a portion thereof, and exons 12-19, of RET. In some embodiments, the BMS1-RET fusion nucleic acid molecule of the disclosure comprises, in 5′ to 3′ direction, exons 1-12, and exon 13 or a portion thereof, of BMS1 fused to exon 10 or a portion thereof, and exons 11-19, of RET. In some embodiments, the CEP135-RET fusion nucleic acid molecule of the disclosure comprises, in 5′ to 3′ direction, exons 1-10, and exon 11 or a portion thereof, of CEP135 fused to exon 12 or a portion thereof, and exons 13-19, of RET. In some embodiments, the EEA1-RET fusion nucleic acid molecule of the disclosure comprises, in 5′ to 3′ direction, exons 1-12, and exon 13 or a portion thereof, of EEA1 fused to exon 12 or a portion thereof, and exons 13-19, of RET. In some embodiments, the CSGALNACT2-RET fusion nucleic acid molecule of the disclosure comprises, in 5′ to 3′ direction, exons 1-5, and exon 6 or a portion thereof, of CSGALNACT2 fused to exon 12 or a portion thereof, and exons 13-19, of RET. In some embodiments, the KIAA1217-RET fusion nucleic acid molecule of the disclosure comprises, in the 5′ to 3′ direction, exons 1-10, and exon 11 or a portion thereof, of KIAA1217 fused to exon 8 or a portion thereof, and exons 9-19, of RET. In some embodiments, the SORBS1-RET fusion nucleic acid molecule of the disclosure comprises, in 5′ to 3′ direction, exons 1-19, and exon 20 or a portion thereof, of SORBS1 fused to exon 7 or a portion thereof, and exons 8-19, of RET. In some embodiments, the SORBS1-RET fusion nucleic acid molecule of the disclosure comprises, in 5′ to 3′ direction, exons 1-21, and exon 22 or a portion thereof, of SORBS1 fused to exon 11 or a portion thereof, and exons 12-19, of RET. In some embodiments, the MPRIP-RET fusion nucleic acid molecule of the disclosure comprises, in 5′ to 3′ direction, exons 1-21, and exon 22 or a portion thereof, of MPRIP fused to exon 12 or a portion thereof, and exons 13-19, of RET. In some embodiments, the TFG-RET fusion nucleic acid molecule of the disclosure comprises, in 5′ to 3′ direction, exons 1-4, and exon 5 or a portion thereof, of TFG fused to exon 11 or a portion thereof, and exons 12-19, of RET. In some embodiments, the SPECC1L-RET fusion nucleic acid molecule of the disclosure comprises, in 5′ to 3′ direction, exons 1-9, and exon 10 or a portion thereof, of SPECC1L fused to exon 12 or a portion thereof, and exons 13-19, of RET. In some embodiments, the REEP3-RET fusion nucleic acid molecule of the disclosure comprises, in 5′ to 3′ direction, exons 1-4, and exon 5 or a portion thereof, of REEP3 fused to exon 12 or a portion thereof, and exons 13-19, of RET. In some embodiments, the RRBP1-RET fusion nucleic acid molecule of the disclosure comprises, in 5′ to 3′ direction, exons 1-21, and exon 22 or a portion thereof, of RRBP1 fused to exon 12 or a portion thereof, and exons 13-19, of RET. In some embodiments, the ETV6-RET fusion nucleic acid molecule of the disclosure comprises, in 5′ to 3′ direction, exons 1-5, and exon 6 or a portion thereof, of ETV6 fused to exon 12 or a portion thereof, and exons 13-19, of RET. In some embodiments, the TAF3-RET fusion nucleic acid molecule of the disclosure comprises, in 5′ to 3″ direction, exons 1-2, and exon 3 or a portion thereof, of TAF3 fused to exon 12 or a portion thereof, and exons 13-19, of RET. In some embodiments, the PCM1-RET fusion nucleic acid molecule of the disclosure comprises, in 5′ to 3′ direction, exons 1-28, and exon 29 or a portion thereof, of PCM1 fused to exon 12 or a portion thereof, and exons 13-19, of RET. In some embodiments, the TNIP2-RET fusion nucleic acid molecule of the disclosure comprises, in 5′ to 3′ direction, exons 1-4, and exon 5 or a portion thereof, of TNIP2 fused to exon 12 or a portion thereof, and exons 13-19, of RET. In some embodiments, the SATB1-RET fusion nucleic acid molecule of the disclosure comprises, in the 5′ to 3′ direction, exons 1-6, and exon 7 or a portion thereof, of SATB1 fused to exon 12 or a portion thereof, and exons 13-19, of RET. In some embodiments, the RET-ADCY1 fusion nucleic acid molecule of the disclosure comprises, in 5′ to 3′ direction, exons 1-10, and exon 11 or a portion thereof, of RET fused to exon 12 or a portion thereof, and exons 13-20, of ADCY1. In some embodiments, the RET-ZNF248 fusion nucleic acid molecule of the disclosure comprises, in 5′ to 3′ direction, exons 1-10, and exon 11 or a portion thereof, of RET fused to exon 4 or a portion thereof, and exons 5-6, of ZNF248. In some embodiments, the RET-AGBL4 fusion nucleic acid molecule of the disclosure comprises, in 5′ to 3′ direction, exons 1-11, and exon 12 or a portion thereof, of RET fused to exon 3 or a portion thereof, and exons 4-14, of AGBL4. In some embodiments, the RET-LRMDA fusion nucleic acid molecule of the disclosure comprises, in 5′ to 3′ direction, exons 1-9, and exon 10 or a portion thereof, of RET fused to exon 6 or a portion thereof, of LRMDA. In some embodiments, the RET-ARHGAP19 fusion nucleic acid molecule of the disclosure comprises, in 5′ to 3′ direction, exons 1-10, and exon 11 or a portion thereof, of RET fused to exon 7 or a portion thereof, and exons 8-12, of ARHGAP19. In some embodiments, the RET-CPEB3 fusion nucleic acid molecule of the disclosure comprises, in 5′ to 3′ direction, exons 1-10, and exon 11 or a portion thereof, of RET fused to exon 8 or a portion thereof, and exons 9-10, of CPEB3. In some embodiments, the RET-DCC fusion nucleic acid molecule of the disclosure comprises, in 5′ to 3′ direction, exons 1-8, and exon 9 or a portion thereof, of RET fused to exon 8 or a portion thereof, and exons 9-29, of DCC. In some embodiments, the RET-ELMO1 fusion nucleic acid molecule of the disclosure comprises, in 5′ to 3′ direction, exons 1-10, and exon 11 or a portion thereof, of RET fused to exon 14 or a portion thereof, and exons 15-22, of ELMO1. In some embodiments, the RET-WDFY4 fusion nucleic acid molecule of the disclosure comprises, in the 5° to 3′ direction, exons 1-14, and exon 15 or a portion thereof, of RET fused to exon 40 or a portion thereof, and exons 41-62, of WDFY4. In some embodiments, the MKX-RET fusion nucleic acid molecule of the disclosure comprises, in 5′ to 3′ direction, exons 1-4, and exon 5 or a portion thereof, of MKX fused to exon 12 or a portion thereof, and exons 13-19, of RET. In some embodiments, the RBMS3-RET fusion nucleic acid molecule of the disclosure comprises, in 5′ to 3′ direction, exons 1-5, and exon 6 or a portion thereof, of RBMS3 fused to exon 12 or a portion thereof, and exons 13-19, of RET. In some embodiments, the SGIP1-RET fusion nucleic acid molecule of the disclosure comprises, in 5′ to 3′ direction, exon 1 or a portion thereof, of SGIP1 fused to exon 12 or a portion thereof, and exons 13-19, of RET. In some embodiments, the ZSWIM6-RET fusion nucleic acid molecule of the disclosure comprises, in 5′ to 3′ direction, exon 1 or a portion thereof, of ZSWIM6 fused to exon 12 or a portion thereof, and exons 13-19, of RET. In some embodiments, the ALOX5-RET fusion nucleic acid molecule of the disclosure comprises, in the 5° to 3′ direction, exon 1, and exon 2 or a portion thereof, of ALOX5 fused to exon 12 or a portion thereof, and exons 13-19, of RET. In some embodiments, the RET-NTRK2 fusion nucleic acid molecule of the disclosure comprises, in 5′ to 3′ direction, exons 1-9, and exon 10 or a portion thereof, of RET fused to exon 20 or a portion thereof, and exon 21, of NTRK2. In some embodiments, the RET-OXR1 fusion nucleic acid molecule of the disclosure comprises, in 5′ to 3′ direction, exons 1-10, and exon 11 or a portion thereof, of RET fused to exon 4 or a portion thereof, and exons 5-16, of OXR1. In some embodiments, the RET-CCBE1 fusion nucleic acid molecule of the disclosure comprises, in the 5′ to 3′ direction, exons 1-7, and exon 8 or a portion thereof, of RET fused to exon 3 or a portion thereof, and exons 4-11, of CCBE1. In some embodiments, the RET-NAALADL2 fusion nucleic acid molecule of the disclosure comprises, in 5′ to 3′ direction, exons 1-10, and exon 11 or a portion thereof, of RET fused to exon 13 or a portion thereof, and exon 14, of NAALADL2. In some embodiments, the RAI14-RET fusion nucleic acid molecule of the disclosure comprises, in the 5° to 3′ direction, exons 1-14, and exon 15 or a portion thereof, of RAI14 fused to exon 11 or a portion thereof, and exons 12-19, of RET. In some embodiments, the ABI3BP-RET fusion nucleic acid molecule of the disclosure comprises, in 5′ to 3′ direction, exons 1-34, and exon 35 or a portion thereof, of ABI3BP fused to exon 12 or a portion thereof, and exons 13-19, of RET. In some embodiments, the LINC00379-RET fusion nucleic acid molecule of the disclosure comprises, in the 5′ to 3′ direction, exon 1, and exon 2 or a portion thereof, of LINC00379 fused to exon 12 or a portion thereof, and exons 13-19, of RET. In some embodiments, the OPTN-RET fusion nucleic acid molecule of the disclosure comprises, in 5′ to 3′ direction, exon 1 or a portion thereof, of OPTN fused to exon 11 or a portion thereof, and exons 12-19, of RET. In some embodiments, the SH2D3A-RET fusion nucleic acid molecule of the disclosure comprises, in 5′ to 3′ direction, exon 1 or a portion thereof, of SH2D3A fused to exon 12 or a portion thereof, and exons 13-19, of RET. In some embodiments, the ZNF721-RET fusion nucleic acid molecule of the disclosure comprises, in 5′ to 3′ direction, exon 1 or a portion thereof, of ZNF721 fused to exon 12 or a portion thereof, and exons 13-19, of RET. In some embodiments, the ADAMTS14-RET fusion nucleic acid molecule of the disclosure comprises, in 5′ to 3′ direction, exons 1-3, and exon 4 or a portion thereof, of ADAMTS14 fused to exon 12 or a portion thereof, and exons 13-19, of RET. In some embodiments, the CSGALNACT2-RET fusion nucleic acid molecule of the disclosure comprises, in 5′ to 3′ direction, exon 1 or a portion thereof, of CSGALNACT2 fused to exon 12 or a portion thereof, and exons 13-19, of RET. In some embodiments, the CPEB3-RET fusion nucleic acid molecule of the disclosure comprises, in 5′ to 3′ direction, exons 1-9, and exon 10 or a portion thereof, of CPEB3 fused to exon 12 or a portion thereof, and exons 13-19, of RET. In some embodiments, the RET-CCNY fusion nucleic acid molecule of the disclosure comprises, in 5′ to 3′ direction, exons 1-10, and exon 11 or a portion thereof, of RET fused to exon 4 or a portion thereof, and exons 5-12, of CCNY. In some embodiments, the RET-RASGEF1A fusion nucleic acid molecule of the disclosure comprises, in 5′ to 3′ direction, exons 1-4, and exon 5 or a portion thereof, of RET fused to exon 2 or a portion thereof, and exons 3-13, of RASGEF1A. In some embodiments, the RET-HSD17B7P2 fusion nucleic acid molecule of the disclosure comprises, in 5′ to 3′ direction, exons 1-10, and exon 11 or a portion thereof, of RET fused to exon 8 or a portion thereof, of HSD17B7P2. In some embodiments, the RET-RAD1 fusion nucleic acid molecule of the disclosure comprises, in 5′ to 3′ direction, exons 1-10, and exon 11 or a portion thereof, of RET fused to exon 6 or a portion thereof, of RAD1. In some embodiments, the RET-VSTM4 fusion nucleic acid molecule of the disclosure comprises, in 5′ to 3′ direction, exons 1-9, and exon 10 or a portion thereof, of RET fused to exon 8 or a portion thereof, of VSTM4. In some embodiments, the ANKRD26-RET fusion nucleic acid molecule of the disclosure comprises, in 5′ to 3′ direction, exons 1-28, and exon 29 or a portion thereof, of ANKRD26 fused to exon 12 or a portion thereof, and exons 13-19, of RET. In some embodiments, the CLIP1-RET fusion nucleic acid molecule of the disclosure comprises, in 5′ to 3′ direction, exons 1-15, and exon 16 or a portion thereof, of CLIP1 fused to exon 12 or a portion thereof, and exons 13-19, of RET. In some embodiments, the DLG5-RET fusion nucleic acid molecule of the disclosure comprises, in 5′ to 3′ direction, exons 1-13, and exon 14 or a portion thereof, of DLG5 fused to exon 12 or a portion thereof, and exons 13-19, of RET. In some embodiments, the DLG5-RET fusion nucleic acid molecule of the disclosure comprises, in 5′ to 3′ direction, exons 1-26, and exon 27 or a portion thereof, of DLG5 fused to exon 8 or a portion thereof, and exons 9-19, of RET. In some embodiments, the ERC1-RET fusion nucleic acid molecule of the disclosure comprises, in 5′ to 3′ direction, exons 1-11, and exon 12 or a portion thereof, of ERC1 fused to exon 12 or a portion thereof, and exons 13-19, of RET. In some embodiments, the ERC1-RET fusion nucleic acid molecule of the disclosure comprises, in 5′ to 3′ direction, exons 1-7, and exon 8 or a portion thereof, of ERC1 fused to exon 12 or a portion thereof, and exons 13-19, of RET. In some embodiments, the ERC1-RET fusion nucleic acid molecule of the disclosure comprises, in the 5′ to 3′ direction, exons 1-11, and exon 12 or a portion thereof, of ERC1 fused to exon 11 or a portion thereof, and exons 12-19, of RET. In some embodiments, the FRMD4A-RET fusion nucleic acid molecule of the disclosure comprises, in 5′ to 3′ direction, exons 1-11, and exon 12 or a portion thereof, of FRMD4A fused to exon 12 or a portion thereof, and exons 13-19, of RET. In some embodiments, the KIAA1468-RET fusion nucleic acid molecule of the disclosure comprises, in 5′ to 3′ direction, exons 1-9, and exon 10 or a portion thereof, of KIAA1468 fused to exon 12 or a portion thereof, and exons 13-19, of RET. In some embodiments, the NCOA4-RET fusion nucleic acid molecule of the disclosure comprises, in 5′ to 3′ direction, exons 1-7, and exon 8 or a portion thereof, of NCOA4 fused to exon 12 or a portion thereof, and exons 13-19, of RET. In some embodiments, the PARD3-RET fusion nucleic acid molecule of the disclosure comprises, in 5′ to 3′ direction, exon 1, and exon 2 or a portion thereof, of PARD3 fused to exon 8 or a portion thereof, and exons 9-19, of RET. In some embodiments, the PARD3-RET fusion nucleic acid molecule of the disclosure comprises, in 5′ to 3′ direction, exons 1-3, and exon 4 or a portion thereof, of PARD3 fused to exon 12 or a portion thereof, and exons 13-19, of RET. In some embodiments, the PRKAR1A-RET fusion nucleic acid molecule of the disclosure comprises, in 5′ to 3′ direction, exons 1-6, and exon 7 or a portion thereof, of PRKAR1A fused to exon 12 or a portion thereof, and exons 13-19, of RET. In some embodiments, the PRKG1-RET fusion nucleic acid molecule of the disclosure comprises, in 5′ to 3′ direction, exons 1-6, and exon 7 or a portion thereof, of PRKG1 fused to exon 4 or a portion thereof, and exons 5-19, of RET. In some embodiments, the PRKG1-RET fusion nucleic acid molecule of the disclosure comprises, in 5′ to 3′ direction, exons 1-4, and exon 5 or a portion thereof, of PRKG1 fused to exon 18 or a portion thereof, and exon 19, of RET. In some embodiments, the RUFY2-RET fusion nucleic acid molecule of the disclosure comprises, in 5′ to 3′ direction, exons 1-8, and exon 9 or a portion thereof, of RUFY2 fused to exon 12 or a portion thereof, and exons 13-19, of RET. In some embodiments, the SNRNP70-RET fusion nucleic acid molecule of the disclosure comprises, in 5′ to 3′ direction, exons 1-5, and exon 6 or a portion thereof, of SNRNP70 fused to exon 12 or a portion thereof, and exons 13-19, of RET. In some embodiments, the SQSTM1-RET fusion nucleic acid molecule of the disclosure comprises, in the 5″ to 3′ direction, exons 1-4, and exon 5 or a portion thereof, of SQSTM1 fused to exon 11 or a portion thereof, and exons 12-19, of RET. In some embodiments, the TNIP1-RET fusion nucleic acid molecule of the disclosure comprises, in 5′ to 3′ direction, exons 1-12, and exon 13 or a portion thereof, of TNIP1 fused to exon 12 or a portion thereof, and exons 13-19, of RET. In some embodiments, the TNIP1-RET fusion nucleic acid molecule of the disclosure comprises, in 5′ to 3′ direction, exons 1-14, and exon 15 or a portion thereof, of TNIP1 fused to exon 12 or a portion thereof, and exons 13-19, of RET. In some embodiments, the TNIP1-RET fusion nucleic acid molecule of the disclosure comprises, in 5′ to 3′ direction, exons 1-11, and exon 12 or a portion thereof, of TNIP1 fused to exon 12 or a portion thereof, and exons 13-19, of RET. In some embodiments, the TRIM27-RET fusion nucleic acid molecule of the disclosure comprises, in the 5° to 3′ direction, exons 1-2, and exon 3 or a portion thereof, of TRIM27 fused to exon 12 or a portion thereof, and exons 13-19, of RET. In some embodiments, the TRIM33-RET fusion nucleic acid molecule of the disclosure comprises, in 5′ to 3′ direction, exons 1-9, and exon 10 or a portion thereof, of TRIM33 fused to exon 12 or a portion thereof, and exons 13-19, of RET. In some embodiments, the CCDC6-RET fusion nucleic acid molecule of the disclosure comprises, in 5′ to 3′ direction, exons 1-2, and exon 3 or a portion thereof, of CCDC6 fused to exon 11 or a portion thereof, and exons 12-19, of RET. In some embodiments, the TRIM24-RET fusion nucleic acid molecule of the disclosure comprises, in 5′ to 3′ direction, exons 1-16, and exon 17 or a portion thereof, of TRIM24 fused to exon 12 or a portion thereof, and exons 13-19, of RET. In some embodiments, the FGFR1OP-RET fusion nucleic acid molecule of the disclosure comprises, in 5′ to 3′ direction, exons 1-5 and exon 6 or a portion thereof, of FGFR1OP fused to exon 12 or a portion thereof, and exons 13-19, of RET. In some embodiments, the GAS2-RET fusion nucleic acid molecule of the disclosure comprises, in 5′ to 3′ direction, exons 1-5, and exon 6 or a portion thereof, of GAS2 fused to exon 4 or a portion thereof, and exons 5-19, of RET. In some embodiments, the HOOK1-RET fusion nucleic acid molecule of the disclosure comprises, in 5′ to 3′ direction, exons 1-19, and exon 20 or a portion thereof, of HOOK1 fused to exon 12 or portion thereof, and exons 13-19, of RET. In some embodiments, the LMNA-RET fusion nucleic acid molecule of the disclosure comprises, in 5′ to 3′ direction, exon 1, and exon 2 or a portion thereof, of LMNA fused to exon 12 or a portion thereof, and exons 13-19, of RET. In some embodiments, the MLPH-RET fusion nucleic acid molecule of the disclosure comprises, in 5′ to 3′ direction, exons 1-8, and exon 9 or a portion thereof, of MLPH fused to exon 12 or a portion thereof, and exons 13-19, of RET. In some embodiments, the MYH9-RET fusion nucleic acid molecule of the disclosure comprises, in the 5′ to 3′ direction, exons 1-33, and exon 34 or a portion thereof, of MYH9 fused to exon 12 or a portion thereof, and exons 13-19, of RET. In some embodiments, the PCDH15-RET fusion nucleic acid molecule of the disclosure comprises, in 5′ to 3′ direction, exon 1 or a portion thereof, of PCDH15 fused to exon 5 or a portion thereof, and exons 6-19, of RET. In some embodiments, the PIBF1-RET fusion nucleic acid molecule of the disclosure comprises, in 5′ to 3′ direction, exons 1-15, and exon 16 or a portion thereof, of PIBF1 fused to exon 12 or a portion thereof, and exons 13-19, of RET. In some embodiments, the SLC12A2-RET fusion nucleic acid molecule of the disclosure comprises, in 5′ to 3′ direction, exons 1-15, and exon 16 or a portion thereof, of SLC12A2 fused to exon 12 or a portion thereof, and exons 13-19, of RET. In some embodiments, the ZNF485-RET fusion nucleic acid molecule of the disclosure comprises, in 5′ to 3′ direction, exons 1-3, and exon 4 or a portion thereof, of ZNF485 fused to exon 12 or a portion thereof, and exons 13-19 of RET. In some embodiments, the ZNF485-RET fusion nucleic acid molecule of the disclosure comprises, in the 5′ to 3′ direction, exons 1-3, and exon 4 or a portion thereof, of ZNF485 fused to exon 10 or a portion thereof, and exons 11-19, of RET.

In some embodiments, a RET fusion nucleic acid molecule of the disclosure comprises the corresponding nucleotide sequence as listed in Table 8, or a nucleotide sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto.

TABLE 8
Exemplary RET fusion nucleic acid molecule nucleotide sequences.

| RET | | |
|---|---|---|
| Fusion | | |
| Nucleic | | |
| Acid | | SEQ |
| Molecule | RET Fusion nucleic acid molecule sequence | ID NO |
| GOLGB1- | ATGCTGAGCCGATTATCAGGATTAGCAAATGTTGTTTTGCATGAATTATCAG | 2 |
| RET | GAGATGATGACACTGATCAGAATATGAGGGCTCCCCTAGACCCTGAATTACA | |
| | CCAAGAATCTGACATGGAATTTAATAATACTACACAAGAAGATGTTCAGGAG | |
| | CGCCTGGCTTATGCAGAGCAATTGGTGGTGGAGCTAAAAGATATTATTAGAC | |
| | AGAAGGATGTTCAACTGCAGCAGAAAGATGAAGCTCTACAGGAAGAGAGAAA | |
| | AGCTGCTGATAACAAAATTAAAAAACTAAAACTTCATGCGAAGGCCAAATTA | |
| | ACTTCTTTGAATAAATACATAGAAGAAATGAAAGCACAAGGAGGGACTGTTC | |
| | TGCCTACAGAACCTCAGTCAGAGGAGCAACTTTCCAAGCATGACAAGAGTTC | |
| | TACAGAGGAAGAGATGGAAATAGAAAAGATAAAACATAAGCTCCAGGAGAAG | |
| | GAGGAACTAATCAGCACTTTGCAAGCCCAGCTTACTCAGGCACAGGCAGAAC | |
| | AACCTGCACAGAGTTCTACAGAGATGGAAGAATTTGTAATGATGAAGCAACA | |
| | GCTCCAGGAGAAGGAAGAATTCATTAGCACTTTACAAGCCCAGCTCAGCCAG | |
| | ACACAGGCAGAGCAAGCTGCACAGCAGGTGGTCCGAGAGAAAGATGCCCGCT | |
| | TTGAAACACAAGTTCGTCTTCATGAAGATGAGCTTCTTCAGTTAGTAACCCA | |
| | GGCAGATGTGGAAACAGAGATGCAACAGAAATTGAGGGTGCTGCAAAGGAAG | |
| | CTTGAGGAACACGAAGAATCCTTGGTGGGCCGTGCTCAGGTCGTTGACTTGC | |
| | TGCAACAGGAGCTGACTGCTGCTGAGCAGAGAAACCAGATTCTCTCTCAGCA | |
| | GTTACAGCAGATGGAAGCTGAGCATAATACTTTGAGGAACACTGTGGAAACA | |
| | GAAAGAGAGGAGTCCAAGATTCTACTGGAAAAGATGGAACTTGAAGTGGCAG | |
| | AGAGAAAATTATCCTTCCATAATCTGCAGGAAGAAATGCATCATCTTTTAGA | |
| | ACAGTTTGAGCAAGCAGGCCAAGCCCAGGCTGAACTAGAGTCTCGGTATAGT | |
| | GCTTTGGAGCAGAAGCACAAAGCAGAAATGGAAGAGAAGACCTCTCATATTT | |
| | TGAGTCTTCAAAAGACTGGACAAGAGCTGCAGTCTGCCTGTGATGCTCTAAA | |
| | GGATCAAAATTCAAAGCTTCTCCAAGATAAGAATGAGCAAGCAGTTCAGTCA | |
| | GCCCAGACCATTCAGCAACTGGAAGATCAGCTCCAGCAAAAATCCAAAGAAA | |
| | TTAGCCAATTTCTAAATAGACTGCCCTTGCAACAACATGAAACAGCATCTCA | |
| | GACTTCTTTCCCAGATGTTTATAATGAGGGCACACAGGAGGATCCAAAGTGG | |
| | GAATTCCCTCGGAAGAACTTGGTTCTTGGAAAAACTCTAGGAGAAGGCGAAT | |
| | TTGGAAAAGTGGTCAAGGCAACGGCCTTCCATCTGAAAGGCAGAGCAGGGTA | |
| | CACCACGGTGGCCGTGAAGATGCTGAAAGAGAACGCCTCCCCGAGTGAGCTG | |
| | CGAGACCTGCTGTCAGAGTTCAACGTCCTGAAGCAGGTCAACCACCCACATG | |
| | TCATCAAATTGTATGGGGCCTGCAGCCAGGATGGCCCGCTCCTCCTCATCGT | |
| | GGAGTACGCCAAATACGGCTCCCTGCGGGGCTTCCTCCGCGAGAGCCGCAAA | |
| | GTGGGGCCTGGCTACCTGGGCAGTGGAGGCAGCCGCAACTCCAGCTCCCTGG | |
| | ACCACCCGGATGAGCGGGCCCTCACCATGGGCGACCTCATCTCATTTGCCTG | |
| | GCAGATCTCACAGGGGATGCAGTATCTGGCCGAGATGAAGCTCGTTCATCGG | |
| | GACTTGGCAGCCAGAAACATCCTGGTAGCTGAGGGGCGGAAGATGAAGATTT | |
| | CGGATTTCGGCTTGTCCCGAGATGTTTATGAAGAGGATTCCTACGTGAAGAG | |
| | GAGCCAGGGTCGGATTCCAGTTAAATGGATGGCAATTGAATCCCTTTTTGAT | |
| | CATATCTACACCACGCAAAGTGATGTATGGTCTTTTGGTGTCCTGCTGTGGG | |
| | AGATCGTGACCCTAGGGGGAAACCCCTATCCTGGGATTCCTCCTGAGCGGCT | |
| | CTTCAACCTTCTGAAGACCGGCCACCGGATGGAGAGGCCAGACAACTGCAGC | |
| | GAGGAGATGTACCGCCTGATGCTGCAATGCTGGAAGCAGGAGCCGGACAAAA | |
| | GGCCGGTGTTTGCGGACATCAGCAAAGACCTGGAGAAGATGATGGTTAAGAG | |
| | GAGAGACTACTTGGACCTTGCGGCGTCCACTCCATCTGACTCCCTGATTTAT | |
| | GACGACGGCCTCTCAGAGGAGGAGACACCGCTGGTGGACTGTAATAATGCCC | |
| | CCCTCCCTCGAGCCCTCCCTTCCACATGGATTGAAAACAAACTCTATGGTAG | |
| | AATTTCCCATGCATTTACTAGATTCTAG | |
| GP2-RET | ATGCCTCACCTTATGGAAAGGATGGTGGGCTCTGGCCTCCTGTGGCTGGCCT | 3 |
| | TGGTCTCCTGCATTCTGACCCAGGCATCTGCAGTGCAGCGAGGTTATGGAAA | |
| | CCCCATTGAAGCCAGTTCGTATGGGCTGGACCTGGACTGCGGAGCTCCTGGC | |
| | ACCCCAGAGGCTCATGTCTGTTTTGACCCCTGTCAGAATTACACCCTCCTGG | |
| | ATGAACCCTTCCGAAGCACAGAGAACTCAGCAGGGTCCCAGGGGTGCGATAA | |
| | AAACATGAGCGGCTGGTACCGCTTTGTAGGGGAAGGAGGAGTAAGGATGTCG | |
| | GAGACCTGTGTCCAGGTGCACCGATGCCAGACAGACGCTCCCATGTGGCTGA | |
| | ATGGGACCCACCCTGCCCTTGGGGATGGCATCACCAACCACACTGCCTGTGC | |
| | CCATTGGAGTGGCAACTGCTGTTTCTGGAAAACAGAGGTGCTGGTGAAGGCC | |
| | TGCCCAGGCGGGTACCATGTGTACCGGTTGGAAGGCACTCCCTGGTGTAATC | |
| | TGAGATACTGCACAGACCCATCCACTGTGGAGGACAAGTGTGAGAAGGCCTG | |
| | CCGCCCCGAGGAGGAGTGCCTTGCCCTCAACAGCACCTGGGGCTGTTTCTGC | |
| | AGACAGGACCTCAATAGTTCTGATCCACTGTGCGACGAGCTGTGCCGCACGG | |
| | TGATCGCAGCCGCTGTCCTCTTCTCCTTCATCGTCTCGGTGCTGCTGTCTGC | |
| | CTTCTGCATCCACTGCTACCACAAGTTTGCCCACAAGCCACCCATCTCCTCA | |
| | GCTGAGATGACCTTCCGGAGGCCCGCCCAGGCCTTCCCGGTCAGCTACTCCT | |
| | CTTCCGGTGCCCGCCGGCCCTCGCTGGACTCCATGGAGAACCAGGTCTCCGT | |
| | GGATGCCTTCAAGATCCTGGAGGATCCAAAGTGGGAATTCCCTCGGAAGAAC | |
| | TTGGTTCTTGGAAAAACTCTAGGAGAAGGCGAATTTGGAAAAGTGGTCAAGG | |
| | CAACGGCCTTCCATCTGAAAGGCAGAGCAGGGTACACCACGGTGGCCGTGAA | |
| | GATGCTGAAAGAGAACGCCTCCCCGAGTGAGCTGCGAGACCTGCTGTCAGAG | |
| | TTCAACGTCCTGAAGCAGGTCAACCACCCACATGTCATCAAATTGTATGGGG | |
| | CCTGCAGCCAGGATGGCCCGCTCCTCCTCATCGTGGAGTACGCCAAATACGG | |
| | CTCCCTGCGGGGCTTCCTCCGCGAGAGCCGCAAAGTGGGGCCTGGCTACCTG | |
| | GGCAGTGGAGGCAGCCGCAACTCCAGCTCCCTGGACCACCCGGATGAGCGGG | |
| | CCCTCACCATGGGCGACCTCATCTCATTTGCCTGGCAGATCTCACAGGGGAT | |
| | GCAGTATCTGGCCGAGATGAAGCTCGTTCATCGGGACTTGGCAGCCAGAAAC | |
| | ATCCTGGTAGCTGAGGGGCGGAAGATGAAGATTTCGGATTTCGGCTTGTCCC | |
| | GAGATGTTTATGAAGAGGATTCCTACGTGAAGAGGAGCCAGGGTCGGATTCC | |
| | AGTTAAATGGATGGCAATTGAATCCCTTTTTGATCATATCTACACCACGCAA | |
| | AGTGATGTATGGTCTTTTGGTGTCCTGCTGTGGGAGATCGTGACCCTAGGGG | |
| | GAAACCCCTATCCTGGGATTCCTCCTGAGCGGCTCTTCAACCTTCTGAAGAC | |
| | CGGCCACCGGATGGAGAGGCCAGACAACTGCAGCGAGGAGATGTACCGCCTG | |
| | ATGCTGCAATGCTGGAAGCAGGAGCCGGACAAAAGGCCGGTGTTTGCGGACA | |
| | TCAGCAAAGACCTGGAGAAGATGATGGTTAAGAGGAGAGACTACTTGGACCT | |
| | TGCGGCGTCCACTCCATCTGACTCCCTGATTTATGACGACGGCCTCTCAGAG | |
| | GAGGAGACACCGCTGGTGGACTGTAATAATGCCCCCCTCCCTCGAGCCCTCC | |
| | CTTCCACATGGATTGAAAACAAACTCTATGGTAGAATTTCCCATGCATTTAC | |
| | TAGATTCTAG | |
| PPP2R5A- | ATGTCGTCGTCGTCGCCGCCGGCGGGGGCTGCCAGCGCCGCCATCTCGGCCT | 4 |
| RET | CGGAGAAAGTGGACGGCTTCACCCGGAAATCGGTCCGCAAGGCGCAGAGGCA | |
| | GAAGCGCTCCCAGGGCTCGTCGCAGTTTCGCAGCCAGGGCAGCCAGGCAGAG | |
| | CTGCACCCGCTGCCCCAGCTCAAAGGCATGTCAGACCCGAACTGGCCTGGAG | |
| | AGAGTCCTGTACCACTCACGAGAGCTGATGGCACTAACACTGGGTTTCCAAG | |
| | ATATCCAAATGATAGTGTATATGCTAACTGGATGCTTTCACCCTCAGCGGCA | |
| | AAATTAATGGACACGTTTGATAGTTAA | |
| GRM7- | ATGGTCCAGCTGAGGAAGCTGCTCCGCGTCCTGACTTTGATGAAGTTCCCCT | 6 |
| RET | GCTGCGTGCTGGAGGTGCTCCTGTGCGCGCTGGCGGCGGCGGCGCGCGGCCA | |
| | GGAGATGTACGCCCCGCACTCAATCCGGATCGAGGGGGACGTCACCCTCGGG | |
| | GGGCTGTTCCCCGTGCACGCCAAGGGTCCCAGCGGAGTGCCCTGCGGCGACA | |
| | TCAAGAGGGAAAACGGGATCCACAGGCTGGAAGCGATGCTCTACGCCCTGGA | |
| | CCAGATCAACAGTGATCCCAACCTACTGCCCAACGTGACGCTGGGCGCGCGG | |
| | ATCCTGGACACTTGTTCCAGGGACACTTACGCGCTCGAACAGTCGCTTACTT | |
| | TCGTCCAGGCGCTCATCCAGAAGGACACCTCCGACGTGCGCTGCACCAACGG | |
| | CGAACCGCCGGTTTTCGTCAAGCCGGAGAAAGTAGTTGGAGTGATTGGGGCT | |
| | TCGGGGAGTTCGGTCTCCATCATGGTAGCCAACATCCTGAGGCTCTTCCAGA | |
| | TCCCCCAGATTAGTTATGCATCAACGGCACCCGAGCTAAGTGATGACCGGCG | |
| | CTATGACTTCTTCTCTCGCGTGGTGCCACCCGATTCCTTCCAAGCCCAGGCC | |
| | ATGGTAGACATTGTAAAGGCCCTAGGCTGGAATTATGTGTCTACCCTCGCAT | |
| | CGGAAGGAAGTTATGGAGAGAAAGGTGTGGAGTCCTTCACGCAGATTTCCAA | |
| | AGAGGCAGGTGGACTCTGCATTGCCCAGTCCGTGAGAATCCCCCAGGAACGC | |
| | AAAGACAGGACCATTGACTTTGATAGAATTATCAAACAGCTCCTGGACACCC | |
| | CCAACTCCAGGGCCGTCGTGATTTTTGCCAACGATGAGGATATAAAGCAGAT | |
| | CCTTGCAGCAGCCAAAAGAGCTGACCAAGTTGGCCATTTTCTTTGGGTGGGA | |
| | TCAGACAGCTGGGGATCCAAAATAAACCCACTGCACCAGCATGAAGATATCG | |
| | CAGAAGGGGCCATCACCATTCAGCCCAAGCGAGCCACGGTGGAAGGGTTTGA | |
| | TGCCTACTTTACGTCCCGTACACTTGAAAACAACAGAAGAAATGTATGGTTT | |
| | GCCGAATACTGGGAGGAAAACTTCAACTGCAAGTTGACGATTAGTGGGTCAA | |
| | AAAAAGAAGACACAGATCGCAAATGCACAGGACAGGAGAGAATTGGAAAAGA | |
| | TTCCAACTATGAGCAGGAGGGTAAAGTCCAGTTCGTGATTGACGCAGTCTAT | |
| | GCTATGGCTCACGCCCTTCACCACATGAACAAGGATCTCTGTGCTGACTACC | |
| | GGGGTGTCTGCCCAGAGATGGAGCAAGCTGGAGGCAAGAAGTTGCTGAAGTA | |
| | TATACGCAATGTTAATTTCAATGGTAGTGCTGGCACTCCAGTGATGTTTAAC | |
| | AAGAACGGGGATGCACCTGGGCGTTATGACATCTTTCAGTACCAGACCACAA | |
| | ACACCAGCAACCCGGGTTACCGTCTGATCGGGCAGTGGACAGACGAACTTCA | |
| | GCTCAATATAGAAGACATGCAGTGGGGTAAAGGAGTCCGAGAGATACCCGCC | |
| | TCAGTGTGCACACTACCATGTAAGCCAGGACAGAGAAAGAAGACACAGAAAG | |
| | GAACTCCTTGCTGTTGGACCTGTGAGCCTTGCGATGGTTACCAGTACCAGTT | |
| | TGATGAGATGACATGCCAGCATTGCCCCTATGACCAGAGGCCCAATGAAAAT | |
| | CGAACCGGATGCCAGGATATTCCCATCATCAAACTGGAGTGGCACTCCCCCT | |
| | GGGCTGTGATTCCTGTCTTCCTGGCAATGTTGGGGATCATTGCCACCATCTT | |
| | TGTCATGGCCACTTTCATCCGCTACAATGACACGCCCATTGTCCGGGCATCT | |
| | GGGCGGGAACTCAGCTATGTTCTTTTGACGGGCATCTTTCTTTGCTACATCA | |
| | TCACTTTCCTGATGATTGCCAAACCAGATGTGGCAGTGTGTTCTTTCCGGCG | |
| | AGTTTTCTTGGGCTTGGGTATGTGCATCAGTTATGCAGCCCTCTTGACGAAA | |
| | ACAAATCGGATTTATCGCATATTTGAGCAGGGCAAGAAATCAGTAACAGCTC | |
| | CCAGACTCATAAGCCCAACATCACAACTGGCAATCACTTCCAGTTTAATATC | |
| | AGTTCAGCTTCTAGGGGTGTTCATTTGGTTTGGTGTTGATCCACCCAACATC | |
| | ATCATAGACTATGATGAACACAAGACAATGAACCCTGAGCAAGCCAGAGGGG | |
| | TTCTCAAGTGTGACATTACAGATCTCCAAATCATTTGCTCCTTGGGATATAG | |
| | CATTCTTCTCATGGTCACATGTACTGTGTATGCCATCAAGACTCGGGGTGTA | |
| | CCCGAGAATTTTAACGAAGCCAAGCCCATTGGATTCACTATGTACACGACAT | |
| | GTATAGTATGGCTTGCCTTCATTCCAATTTTTTTTGGCACCGCTCAATCAGC | |
| | GGAAAAGCTCTACATACAAACTACCACGCTTACAATCTCCATGAACCTAAGT | |
| | GCATCAGTGGCGCTGGGGATGCTATACATGCCGAAAGTGTACATCATCATTT | |
| | TCCACCCTGAACTCAATGTCCAGAAACGGAAGCGAAGCTTCAAGGCGGTAGT | |
| | CACAGCAGCCACCATGTCATCGAGGCTGTCACACAAACCCAGTGACAGACCC | |
| | AACGGTGAGGCAAAGACCGAGCTCTGTGAAAACGTAGACCCAAACAACCGCG | |
| | GCTTTCCCCTGCTCACCGTCTACCTCAAGGTCTTCCTGTCACCCACATCCCT | |
| | TCGTGAGGGCGAGTGCCAGTGGCCAGGCTGTGCCCGCGTATACTTCTCCTTC | |
| | TTCAACACCTCCTTTCCAGCCTGCAGCTCCCTCAAGCCCCGGGAGCTCTGCT | |
| | TCCCAGAGACAAGGCCCTCCTTCCGCATTCGGGAGAACCGACCCCCAGGCAC | |
| | CTTCCACCAGTTCCGCCTGCTGCCTGTGCAGTTCTTGTGCCCCAACATCAGC | |
| | GTGGCCTACAGGCTCCTGGAGGGTGAGGGTCTGCCCTTCCGCTGCGCCCCGG | |
| | ACAGCCTGGAGGTGAGCACGCGCTGGGCCCTGGACCGCGAGCAGCGGGAGAA | |
| | GTACGAGCTGGTGGCCGTGTGCACCGTGCACGCCGGCGCGCGCGAGGAGGTG | |
| | GTGATGGTGCCCTTCCCGGTGACCGTGTACGACGAGGACGACTCGGCGCCCA | |
| | CCTTCCCCGCGGGCGTCGACACCGCCAGCGCCGTGGTGGAGTTCAAGCGGAA | |
| | GGAGGACACCGTGGTGGCCACGCTGCGTGTCTTCGATGCAGACGTGGTACCT | |
| | GCATCAGGGGAGCTGGTGAGGCGGTACACAAGCACGCTGCTCCCCGGGGACA | |
| | CCTGGGCCCAGCAGACCTTCCGGGTGGAACACTGGCCCAACGAGACCTCGGT | |
| | CCAGGCCAACGGCAGCTTCGTGCGGGCGACCGTACATGACTATAGGCTGGTT | |
| | CTCAACCGGAACCTCTCCATCTCGGAGAACCGCACCATGCAGCTGGCGGTGC | |
| | TGGTCAATGACTCAGACTTCCAGGGCCCAGGAGCGGGCGTCCTCTTGCTCCA | |
| | CTTCAACGTGTCGGTGCTGCCGGTCAGCCTGCACCTGCCCAGTACCTACTCC | |
| | CTCTCCGTGAGCAGGAGGGCTCGCCGATTTGCCCAGATCGGGAAAGTCTGTG | |
| | TGGAAAACTGCCAGGCATTCAGTGGCATCAACGTCCAGTACAAGCTGCATTC | |
| | CTCTGGTGCCAACTGCAGCACGCTAGGGGTGGTCACCTCAGCCGAGGACACC | |
| | TCGGGGATCCTGTTTGTGAATGACACCAAGGCCCTGCGGCGGCCCAAGTGTG | |
| | CCGAACTTCACTACATGGTGGTGGCCACCGACCAGCAGACCTCTAGGCAGGC | |
| | CCAGGCCCAGCTGCTTGTAACAGTGGAGGGGTCATATGTGGCCGAGGAGGCG | |
| | GGCTGCCCCCTGTCCTGTGCAGTCAGCAAGAGACGGCTGGAGTGTGAGGAGT | |
| | GTGGCGGCCTGGGCTCCCCAACAGGCAGGTGTGAGTGGAGGCAAGGAGATGG | |
| | CAAAGGGATCACCAGGAACTTCTCCACCTGCTCTCCCAGCACCAAGACCTGC | |
| | CCCGACGGCCACTGCGATGTTGTGGAGACCCAAGACATCAACATTTGCCCTC | |
| | AGGACTGCCTCCGGGGCAGCATTGTTGGGGGACACGAGCCTGGGGAGCCCCG | |
| | GGGGATTAAAGCTGGCTATGGCACCTGCAACTGCTTCCCTGAGGAGGAGAAG | |
| | TGCTTCTGCGAGCCCGAAGACATCCAGGATCCACTGTGCGACGAGCTGTGCC | |
| | GCACGGTGATCGCAGCCGCTGTCCTCTTCTCCTTCATCGTCTCGGTGCTGCT | |
| | GTCTGCCTTCTGCATCCACTGCTACCACAAGTTTGCCCACAAGCCACCCATC | |
| | TCCTCAGCTGAGATGACCTTCCGGAGGCCCGCCCAGGCCTTCCCGGTCAGCT | |
| | ACTCCTCTTCCGGTGCCCGCCGGCCCTCGCTGGACTCCATGGAGAACCAGGT | |
| | CTCCGTGGATGCCTTCAAGATCCTGGAGGATCCAAAGTGGGAATTCCCTCGG | |
| | AAGAACTTGGTTCTTGGAAAAACTCTAGGAGAAGGCGAATTTGGAAAAGTGG | |
| | TCAAGGCAACGGCCTTCCATCTGAAAGGCAGAGCAGGGTACACCACGGTGGC | |
| | CGTGAAGATGCTGAAAGAGAACGCCTCCCCGAGTGAGCTGCGAGACCTGCTG | |
| | TCAGAGTTCAACGTCCTGAAGCAGGTCAACCACCCACATGTCATCAAATTGT | |
| | ATGGGGCCTGCAGCCAGGATGGCCCGCTCCTCCTCATCGTGGAGTACGCCAA | |
| | ATACGGCTCCCTGCGGGGCTTCCTCCGCGAGAGCCGCAAAGTGGGGCCTGGC | |
| | TACCTGGGCAGTGGAGGCAGCCGCAACTCCAGCTCCCTGGACCACCCGGATG | |
| | AGCGGGCCCTCACCATGGGCGACCTCATCTCATTTGCCTGGCAGATCTCACA | |
| | GGGGATGCAGTATCTGGCCGAGATGAAGCTCGTTCATCGGGACTTGGCAGCC | |
| | AGAAACATCCTGGTAGCTGAGGGGCGGAAGATGAAGATTTCGGATTTCGGCT | |
| | TGTCCCGAGATGTTTATGAAGAGGATTCCTACGTGAAGAGGAGCCAGGGTCG | |
| | GATTCCAGTTAAATGGATGGCAATTGAATCCCTTTTTGATCATATCTACACC | |
| | ACGCAAAGTGATGTATGGTCTTTTGGTGTCCTGCTGTGGGAGATCGTGACCC | |
| | TAGGGGGAAACCCCTATCCTGGGATTCCTCCTGAGCGGCTCTTCAACCTTCT | |
| | GAAGACCGGCCACCGGATGGAGAGGCCAGACAACTGCAGCGAGGAGATGTAC | |
| | CGCCTGATGCTGCAATGCTGGAAGCAGGAGCCGGACAAAAGGCCGGTGTTTG | |
| | CGGACATCAGCAAAGACCTGGAGAAGATGATGGTTAAGAGGAGAGACTACTT | |
| | GGACCTTGCGGCGTCCACTCCATCTGACTCCCTGATTTATGACGACGGCCTC | |
| | TCAGAGGAGGAGACACCGCTGGTGGACTGTAATAATGCCCCCCTCCCTCGAG | |
| | CCCTCCCTTCCACATGGATTGAAAACAAACTCTATGGTAGAATTTCCCATGC | |
| | ATTTACTAGATTCTAG | |
| MYH14- | ATGGCAGCCGTGACCATGTCGGTGCCCGGGCGGAAGGCGCCCCCCAGGCCGG | 8 |
| RET | GCCCAGTGCCCGAGGCGGCCCAGCCGTTCCTGTTCACGCCCCGCGGGCCCAG | |
| | CGCGGGTGGCGGGCCTGGCTCGGGCACCTCCCCGCAGGTGGAGTGGACGGCC | |
| | CGGCGTCTCGTGTGGGTGCCTTCGGAGCTTCACGGGTTCGAGGCGGCGGCGC | |
| | TGCGGGACGAAGGCGAGGAGGAGGCGGAGGTGGAGCTGGCGGAGAGCGGGAG | |
| | GCGGCTGCGACTGCCGCGGGACCAGATCCAGCGCATGAACCCGCCCAAGTTC | |
| | AGCAAGGCCGAGGACATGGCCGAGCTGACCTGCCTCAACGAGGCCTCGGTCC | |
| | TGCACAACCTCCGGGAGCGGTACTACTCCGGCCTCATCTACACGTACTCCGG | |
| | CCTTTTCTGTGTGGTCATCAACCCGTACAAGCAGCTTCCCATCTACACAGAA | |
| | GCCATTGTGGAGATGTACCGGGGCAAGAAGCGCCACGAGGTGCCACCCCACG | |
| | TGTACGCAGTGACCGAGGGGGCCTATCGGAGCATGCTGCAGGATCGTGAGGA | |
| | CCAGTCCATTCTCTGCACTGGAGAGTCTGGAGCTGGGAAGACGGAAAACACC | |
| | AAGAAGGTCATCCAGTACCTCGCCCACGTGGCGTCGTCTCCAAAGGGCAGGA | |
| | AGGAGCCGGGTGTCCCCGGTGAGCTGGAGCGGCAGCTGCTTCAGGCCAACCC | |
| | CATCCTAGAGGCCTTTGGCAATGCCAAGACAGTGAAGAATGACAACTCCTCC | |
| | CGATTCGGCAAATTCATCCGCATCAACTTTGATGTTGCCGGGTACATCGTGG | |
| | GCGCCAACATTGAGACCTACCTGCTGGAGAAGTCGCGGGCCATCCGCCAGGC | |
| | CAAGGACGAGTGCAGCTTCCACATCTTCTACCAGCTGCTGGGGGGCGCTGGA | |
| | GAGCAGCTCAAAGCCGACCTCCTCCTCGAGCCCTGCTCCCACTACCGGTTCC | |
| | TGACCAACGGGCCGTCATCCTCTCCCGGCCAGGAGCGGGAACTCTTCCAGGA | |
| | GACGCTGGAGTCGCTGCGGGTCCTGGGATTCAGCCACGAGGAAATCATCTCC | |
| | ATGCTGCGGATGGTCTCAGCAGTTCTCCAGTTTGGCAACATTGCCTTGAAGA | |
| | GAGAACGGAACACCGATCAAGCCACCATGCCTGACAACACAGCTGCACAGAA | |
| | GCTCTGCCGCCTCTTGGGACTGGGGGTGACGGATTTCTCCCGAGCCTTGCTC | |
| | ACCCCTCGCATCAAAGTTGGCCGAGACTATGTGCAGAAAGCCCAGACTAAGG | |
| | AACAGGCTGACTTCGCGCTGGAGGCCCTGGCCAAGGCCACCTACGAGCGCCT | |
| | CTTCCGCTGGCTGGTTCTGCGCCTCAACCGGGCCTTGGACCGCAGCCCCCGC | |
| | CAAGGCGCCTCCTTCCTGGGCATCCTGGACATCGCGGGCTTTGAGATCTTCC | |
| | AGCTGAACTCCTTCGAGCAGCTCTGCATCAACTACACCAACGAGAAGCTGCA | |
| | GCAGCTCTTCAACCACACCATGTTCGTGCTGGAGCAGGAGGAGTACCAGCGT | |
| | GAGGGCATCCCCTGGACCTTCCTCGACTTTGGCCTCGACCTGCAGCCCTGCA | |
| | TCGACCTCATCGAGCGGCCGGCCAACCCCCCTGGACTCCTGGCCCTGCTGGA | |
| | TGAGGAGTGCTGGTTCCCGAAGGCCACAGACAAGTCGTTTGTGGAGAAGGTA | |
| | GCCCAGGAGCAGGGCGGCCACCCCAAGTTCCAGCGGCCGAGGCACCTGCGGG | |
| | ATCAGGCCGACTTCAGTGTTCTCCACTACGCGGGCAAGGTCGACTACAAGGC | |
| | CAACGAGTGGCTGATGAAAAACATGGACCCTCTGAATGACAACGTCGCAGCC | |
| | TTGCTCCACCAGAGCACAGACCGGCTGACGGCAGAGATCTGGAAAGACGTGG | |
| | AGGGCATCGTGGGGCTGGAACAGGTGAGCAGCCTGGGCGACGGCCCACCAGG | |
| | TGGCCGCCCCCGTCGGGGTATGTTCCGGACAGTGGGACAGCTCTACAAGGAG | |
| | TCCCTGAGCCGCCTCATGGCCACACTCAGCAACACCAACCCCAGTTTTGTCC | |
| | GCTGCATTGTCCCCAACCACGAGAAGAGGGCCGGGAAGCTGGAGCCACGGCT | |
| | GGTGCTGGACCAGCTTCGCTGCAACGGGGTCCTGGAGGGCATCCGCATCTGT | |
| | CGCCAGGGCTTCCCCAACCGCATCCTCTTCCAGGAGTTCCGGCAGCGATACG | |
| | AGATCCTGACACCCAATGCCATCCCCAAGGGCTTCATGGATGGGAAGCAGGC | |
| | CTGTGAAAAGATGATCCAGGCGCTGGAACTGGACCCCAACCTCTACCGCGTG | |
| | GGACAGAGCAAGATCTTCTTCCGGGCTGGGGTCCTGGCCCAGCTGGAAGAGG | |
| | AGCGAGACCTGAAGGTCACCGACATCATCGTCTCCTTCCAGGCAGCTGCCCG | |
| | GGGATACCTGGCTCGCAGGGCCTTCCAGAAGCGCCAGCAGCAGCAGAGCGCC | |
| | CTGAGGGTGATGCAGCGGAACTGCGCGGCCTACCTCAAGCTGAGACACTGGC | |
| | AGTGGTGGCGGCTGTTTACCAAGGTGAAGCCACTGCTGCAGGTGACGCGGCA | |
| | GGATGAGGTGCTGCAGGCACGGGCCCAGGAGCTGCAGAAAGTGCAGGAGCTA | |
| | CAGCAGCAGAGCGCCCGCGAAGTTGGGGAGCTCCAGGGCCGAGTGGCACAGC | |
| | TGGAAGAGGAGCGCGCCCGCCTGGCAGAGCAATTGCGAGCAGAGGCAGAACT | |
| | GTGTGCAGAGGCCGAGGAGACGCGGGGGAGGCTGGCAGCCCGCAAGCAGGAG | |
| | CTGGAGCTGGTGGTGTCAGAGCTGGAGGCTCGCGTGGGCGAGGAGGAGGAGT | |
| | GCAGCCGTCAAATGCAAACCGAGAAGAAGAGGCTGCAGCAGCACATACAGGA | |
| | GCTAGAGGCCCACCTTGAGGCTGAGGAGGGTGCGCGGCAGAAGCTGCAGCTG | |
| | GAGAAGGTGACGACAGAGGCAAAAATGAAGAAATTTGAAGAGGACCTGCTGC | |
| | TCCTGGAAGACCAGAATTCCAAGCTGAGCAAGGAGCGGAAGCTGCTGGAAGA | |
| | TCGTCTGGCCGAGTTCTCATCCCAGGCAGCTGAGGAGGAGGAGAAGGTCAAG | |
| | AGCCTCAATAAGCTACGGCTCAAATATGAGGCCACAATCGCAGACATGGAGG | |
| | ACCGCCTACGGAAGGAGGAGAAGGGTCGCCAGGAGCTGGAGAAGCTGAAGCG | |
| | GAGGCTGGATGGGGAGAGCTCAGAGCTGCAGGAGCAGATGGTGGAGCAGCAA | |
| | CAGCGGGCAGAGGAGCTGCGGGCCCAGCTGGGCCGGAAGGAGGAGGAGCTGC | |
| | AGGCTGCCCTGGCCAGGGCAGAAGACGAGGGTGGGGCCCGGGCCCAGCTGCT | |
| | GAAATCCCTGCGGGAGGCTCAAGCAGCCCTGGCCGAGGCCCAGGAGGACCTG | |
| | GAGTCTGAGCGTGTGGCCAGGACCAAGGCGGAGAAGCAGCGCCGGGACCTGG | |
| | GCGAGGAGCTGGAGGCGCTGCGGGGCGAGCTGGAGGACACGCTGGACTCCAC | |
| | CAACGCACAGCAGGAGCTCCGGTCCAAGAGGGAACAGGAGGTGACGGAGCTG | |
| | AAGAAGACTCTGGAGGAGGAGACTCGCATCCACGAGGCGGCAGTGCAGGAGC | |
| | TGAGGCAGCGCCACGGCCAGGCCCTGGGGGAGCTGGCGGAGCAGCTGGAGCA | |
| | GGCCCGGAGGGGCAAAGGTGCATGGGAGAAGACCCGGCTGGCCCTGGAGGCC | |
| | GAGGTGTCCGAGCTGCGGGCAGAACTGAGCAGCCTGCAGACTGCACGTCAGG | |
| | AGGGTGAGCAGCGGAGGCGCCGCCTGGAGTTACAGCTGCAGGAGGTGCAGGG | |
| | CCGGGCTGGTGATGGGGAGAGGGCACGAGCGGAGGCTGCTGAGAAGCTGCAG | |
| | CGAGCCCAGGCTGAACTGGAGAATGTGTCTGGGGCGCTGAACGAGGCTGAGT | |
| | CCAAAACCATCCGTCTTAGCAAGGAGCTGAGCAGCACAGAAGCCCAGCTGCA | |
| | CGATGCCCAGGAGCTGCTGCAGGAGGAGACCAGGGCGAAATTGGCCTTGGGG | |
| | TCCCGGGTGCGAGCCATGGAGGCTGAGGCAGCCGGGCTGCGTGAGCAGCTGG | |
| | AGGAGGAGGCAGCTGCCAGGGAACGGGCGGGCCGTGAACTGCAGACTGCCCA | |
| | GGCCCAGCTTTCCGAGTGGCGGCGGCGCCAGGAGGAGGAGGCAGGGGCACTG | |
| | GAGGCAGGGGAGGAGGCACGGCGCCGGGCAGCCCGGGAGGCCGAGGCCCTGA | |
| | CCCAGCGCCTGGCAGAAAAGACAGAGACCGTGGATCGGCTGGAGCGGGGCCG | |
| | CCGCCGGCTGCAGCAGGAGCTGGACGACGCCACCATGGACCTGGAGCAGCAG | |
| | CGGCAGCTTGTGAGCACCCTGGAGAAGAAGCAGCGCAAGTTTGACCAGCTTC | |
| | TGGCAGAGGAGAAGGCAGCTGTACTTCGGGCAGTGGAGGAACGTGAGCGGGC | |
| | CGAGGCAGAGGGCCGGGAGCGTGAGGCTCGGGCCCTGTCACTGACACGGGCA | |
| | CTGGAGGAGGAGCAGGAGGCACGTGAGGAGCTGGAGCGGCAGAACCGGGCCC | |
| | TGCGGGCTGAGCTGGAGGCACTGCTGAGCAGCAAGGATGACGTCGGCAAGAG | |
| | CGTGCATGAGCTGGAACGAGCCTGCCGGGTAGCAGAACAGGCAGCCAATGAT | |
| | CTGCGAGCACAGGTGACAGAACTGGAGGATGAGCTGACAGCGGCCGAGGATG | |
| | CCAAGCTGCGTCTGGAGGTGACTGTGCAGGCTCTCAAGACTCAGCATGAGCG | |
| | TGACCTGCAGGGCCGTGATGAGGCTGGTGAAGAGAGGCGGAGGCAGCTGGCC | |
| | AAGCAGCTGAGAGATGCAGAGGTGGAGCGGGATGAGGAGCGGAAGCAGCGCA | |
| | CTCTGGCCGTGGCTGCCCGCAAGAAGCTGGAGGGAGAGCTGGAGGAGCTGAA | |
| | GGCTCAGATGGCCTCTGCCGGCCAGGGCAAGGAGGAGGCGGTGAAGCAGCTT | |
| | CGCAAGATGCAGGCCCAGATGAAGGAGCTATGGCGGGAGGTGGAGGAGACAC | |
| | GCACCTCCCGGGAGGAGATCTTCTCCCAGAATCGGGAAAGTGAAAAGCGCCT | |
| | CAAGGGCCTGGAGGCTGAGGTGCTGCGGCTGCAGGAGGAACTGGCCGCCTCG | |
| | GACCGTGCTCGGCGGCAGGCCCAGCAGGACCGGGATGAGATGGCAGATGAGG | |
| | TGGCCAATGGTAACCTTAGCAAGGCAGCCATTCTGGAGGAGAAGCGTCAGCT | |
| | GGAGGGGCGCCTGGGGCAGTTGGAGGAAGAGCTGGAGGAGGAGCAGAGCAAC | |
| | TCGGAGCTGCTCAATGACCGCTACCGCAAGCTGCTCCTGCAGGAGGATCCAA | |
| | AGTGGGAATTCCCTCGGAAGAACTTGGTTCTTGGAAAAACTCTAGGAGAAGG | |
| | CGAATTTGGAAAAGTGGTCAAGGCAACGGCCTTCCATCTGAAAGGCAGAGCA | |
| | GGGTACACCACGGTGGCCGTGAAGATGCTGAAAGAGAACGCCTCCCCGAGTG | |
| | AGCTGCGAGACCTGCTGTCAGAGTTCAACGTCCTGAAGCAGGTCAACCACCC | |
| | ACATGTCATCAAATTGTATGGGGCCTGCAGCCAGGATGGCCCGCTCCTCCTC | |
| | ATCGTGGAGTACGCCAAATACGGCTCCCTGCGGGGCTTCCTCCGCGAGAGCC | |
| | GCAAAGTGGGGCCTGGCTACCTGGGCAGTGGAGGCAGCCGCAACTCCAGCTC | |
| | CCTGGACCACCCGGATGAGCGGGCCCTCACCATGGGCGACCTCATCTCATTT | |
| | GCCTGGCAGATCTCACAGGGGATGCAGTATCTGGCCGAGATGAAGCTCGTTC | |
| | ATCGGGACTTGGCAGCCAGAAACATCCTGGTAGCTGAGGGGCGGAAGATGAA | |
| | GATTTCGGATTTCGGCTTGTCCCGAGATGTTTATGAAGAGGATTCCTACGTG | |
| | AAGAGGAGCCAGGGTCGGATTCCAGTTAAATGGATGGCAATTGAATCCCTTT | |
| | TTGATCATATCTACACCACGCAAAGTGATGTATGGTCTTTTGGTGTCCTGCT | |
| | GTGGGAGATCGTGACCCTAGGGGGAAACCCCTATCCTGGGATTCCTCCTGAG | |
| | CGGCTCTTCAACCTTCTGAAGACCGGCCACCGGATGGAGAGGCCAGACAACT | |
| | GCAGCGAGGAGATGTACCGCCTGATGCTGCAATGCTGGAAGCAGGAGCCGGA | |
| | CAAAAGGCCGGTGTTTGCGGACATCAGCAAAGACCTGGAGAAGATGATGGTT | |
| | AAGAGGAGAGACTACTTGGACCTTGCGGCGTCCACTCCATCTGACTCCCTGA | |
| | TTTATGACGACGGCCTCTCAGAGGAGGAGACACCGCTGGTGGACTGTAATAA | |
| | TGCCCCCCTCCCTCGAGCCCTCCCTTCCACATGGATTGAAAACAAACTCTAT | |
| | GGTAGAATTTCCCATGCATTTACTAGATTCTAG | |
| MGEA5- | ATGGTGCAGAAGGAGAGTCAAGCGACGTTGGAGGAGCGGGAGAGCGAGCTCA | 9 |
| RET | GCTCCAACCCTGCCGCCTCTGCGGGGGCATCGCTGGAGCCGCCGGCAGCTCC | |
| | GGCACCCGGAGAAGACAACCCCGCCGGGGCTGGGGGAGCGGCGGTGGCCGGG | |
| | GCTGCAGGAGGGGCTCGGCGGTTCCTCTGCGGTGTGGTGGAAGGATTTTATG | |
| | GAAGACCTTGGGTTATGGAACAGAGAAAAGAACTCTTTAGAAGGCTCCAGAA | |
| | ATGGGAATTAAATACATACTTGTATGCCCCAAAAGATGACTACAAACATAGG | |
| | ATGTTTTGGCGAGAGATGTATTCAGTGGAGGAAGCTGAGCAACTTATGACTC | |
| | TCATCTCTGCTGCACGAGAATATGAGATAGAGTTCATCTATGCGATCTCACC | |
| | TGGATTGGATATCACTTTTTCTAACCCCAAGGAAGTATCCACATTGAAACGT | |
| | AAATTGGACCAGGTTTCTCAGTTTGGGTGCAGATCATTTGCTTTGCTTTTTG | |
| | ATGATATAGACCATAATATGTGTGCAGCAGACAAAGAGGTATTCAGTTCTTT | |
| | TGCTCATGCCCAAGTCTCCATCACAAATGAAATCTATCAGTACCTAGGAGAG | |
| | CCAGAAACTTTCCTCTTCTGTCCCACAGAATACTGTGGCACTTTCTGTTATC | |
| | CAAATGTGTCTCAGTCTCCATATTTAAGGACTGTGGGTGAAAAGCTTCTACC | |
| | TGGAATTGAAGTGCTTTGGACAGGTCCCAAAGTTGTTTCTAAAGAAATTCCA | |
| | GTAGAGTCCATCGAAGAGGTTTCTAAGATTATTAAGAGAGCTCCAGTAATCT | |
| | GGGATAACATTCATGCTAATGATTATGATCAGAAGAGACTGTTTCTGGGCCC | |
| | GTACAAAGGAAGATCCACAGAACTCATCCCACGGTTAAAAGGAGTCCTCACT | |
| | AATCCAAATTGTGAATTTGAAGCCAACTACGTTGCTATCCACACCCTTGCCA | |
| | CCTGGTACAAATCAAACATGAATGGAGTGAGAAAAGATGTAGTGATGACTGA | |
| | CAGTGAAGATAGTACTGTGTCCATCCAGATAAAATTAGAAAATGAAGGCAGT | |
| | GATGAAGATATTGAAACTGATGTACTCTATAGTCCACAGATGGCTCTAAAGC | |
| | TAGCATTAACAGAATGGTTGCAAGAGTTTGGTGTGCCTCATCAATACAGCAG | |
| | TAGGCAAGTTGCACACAGTGGAGCTAAAGCAAGTGTAGTTGATGGGACTCCT | |
| | TTAGTTGCAGCACCCTCTTTAAATGCCACAACCGTAGTAACAACAGTTTATC | |
| | AGGAGCCCATTATGAGCCAGGGAGCAGCCTTGAGTGGTGAGCCTACTACTCT | |
| | GACCAAGGAAGAAGAAAAGAAACAGCCTGATGAAGAACCCATGGACATGGTG | |
| | GTGGAAAAACAAGAAGAAACGGACCACAAGAATGACAATCAAATACTGAGTG | |
| | AAATTGTTGAAGCGAAAATGGCAGAGGAATTGAAACCAATGGACACTGATAA | |
| | AGAGAGCATAGCTGAATCAAAATCCCCAGAGATGTCCATGCAAGAAGATTGT | |
| | ATTAGTGACATTGCCCCCATGCAAACTGATGAACAGACAAACAAGGAGCAGT | |
| | TTGTGCCAGGTCCAAATGAAAAGCCTTTGTACACTGCGGAACCAGTGACCCT | |
| | GGAGGATTTGCAGTTACTTGCTGATCTATTCTACCTTCCTTACGAGCATGGA | |
| | CCCAAAGGAGCACAGATGTTACGGGAATTTCAATGGCTTCGAGCAAATAGTA | |
| | GTGTTGTCAGTGTCAATTGCAAAGGAAAAGACTCTGAAAAAATTGAAGAATG | |
| | GCGGTCACGAGCAGCCAAGTTTGAAGAGATGTGTGGACTAGTGATGGGAATG | |
| | TTCACTCGGCTCTCCAATTGTGCCAACAGGACAATTCTTTATGACATGTACT | |
| | CCTATGTTTGGGATATCAAGAGTATAATGTCTATGGTGAAGTCTTTTGTACA | |
| | GTGGTTAGGGTGTCGTAGTCATTCTTCAGCACAATTCTTAATTGGAGACCAA | |
| | GAACCCTGGGCCTTTAGAGGTGGTCTAGCAGGAGAGTTCCAGCGTTTGCTGC | |
| | CAATTGATGGGGCAAATGATCTCTTTTTTCAGCCACCTCCACTGACTCCTAC | |
| | CTCCAAAGTTTATACTATCAGACCTTATTTTCCTAAGGATGAGGAGGATCCA | |
| | AAGTGGGAATTCCCTCGGAAGAACTTGGTTCTTGGAAAAACTCTAGGAGAAG | |
| | GCGAATTTGGAAAAGTGGTCAAGGCAACGGCCTTCCATCTGAAAGGCAGAGC | |
| | AGGGTACACCACGGTGGCCGTGAAGATGCTGAAAGAGAACGCCTCCCCGAGT | |
| | GAGCTGCGAGACCTGCTGTCAGAGTTCAACGTCCTGAAGCAGGTCAACCACC | |
| | CACATGTCATCAAATTGTATGGGGCCTGCAGCCAGGATGGCCCGCTCCTCCT | |
| | CATCGTGGAGTACGCCAAATACGGCTCCCTGCGGGGCTTCCTCCGCGAGAGC | |
| | CGCAAAGTGGGGCCTGGCTACCTGGGCAGTGGAGGCAGCCGCAACTCCAGCT | |
| | CCCTGGACCACCCGGATGAGCGGGCCCTCACCATGGGCGACCTCATCTCATT | |
| | TGCCTGGCAGATCTCACAGGGGATGCAGTATCTGGCCGAGATGAAGCTCGTT | |
| | CATCGGGACTTGGCAGCCAGAAACATCCTGGTAGCTGAGGGGCGGAAGATGA | |
| | AGATTTCGGATTTCGGCTTGTCCCGAGATGTTTATGAAGAGGATTCCTACGT | |
| | GAAGAGGAGCCAGGGTCGGATTCCAGTTAAATGGATGGCAATTGAATCCCTT | |
| | TTTGATCATATCTACACCACGCAAAGTGATGTATGGTCTTTTGGTGTCCTGC | |
| | TGTGGGAGATCGTGACCCTAGGGGGAAACCCCTATCCTGGGATTCCTCCTGA | |
| | GCGGCTCTTCAACCTTCTGAAGACCGGCCACCGGATGGAGAGGCCAGACAAC | |
| | TGCAGCGAGGAGATGTACCGCCTGATGCTGCAATGCTGGAAGCAGGAGCCGG | |
| | ACAAAAGGCCGGTGTTTGCGGACATCAGCAAAGACCTGGAGAAGATGATGGT | |
| | TAAGAGGAGAGACTACTTGGACCTTGCGGCGTCCACTCCATCTGACTCCCTG | |
| | ATTTATGACGACGGCCTCTCAGAGGAGGAGACACCGCTGGTGGACTGTAATA | |
| | ATGCCCCCCTCCCTCGAGCCCTCCCTTCCACATGGATTGAAAACAAACTCTA | |
| | TGGTAGAATTTCCCATGCATTTACTAGATTCTAG | |
| SAMD4A- | ATGATGTTTCGCGACCAGGTCGGGGTGCTGGCGGGCTGGTTTAAGGGCTGGA | 10 |
| RET | ACGAGTGCGAGCAGACTGTTGCGCTGCTGTCGCTGCTCAAGCGCGTGAGCCA | |
| | GACCCAGGCCCGCTTCCTCCAGCTCTGCCTGGAGCACTCGCTGGCCGACTGC | |
| | GCCGAGCTGCACGTCCTCGAACGCGAGGCCAACAGCCCCGATCCACTGTGCG | |
| | ACGAGCTGTGCCGCACGGTGATCGCAGCCGCTGTCCTCTTCTCCTTCATCGT | |
| | CTCGGTGCTGCTGTCTGCCTTCTGCATCCACTGCTACCACAAGTTTGCCCAC | |
| | AAGCCACCCATCTCCTCAGCTGAGATGACCTTCCGGAGGCCCGCCCAGGCCT | |
| | TCCCGGTCAGCTACTCCTCTTCCGGTGCCCGCCGGCCCTCGCTGGACTCCAT | |
| | GGAGAACCAGGTCTCCGTGGATGCCTTCAAGATCCTGGAGGATCCAAAGTGG | |
| | GAATTCCCTCGGAAGAACTTGGTTCTTGGAAAAACTCTAGGAGAAGGCGAAT | |
| | TTGGAAAAGTGGTCAAGGCAACGGCCTTCCATCTGAAAGGCAGAGCAGGGTA | |
| | CACCACGGTGGCCGTGAAGATGCTGAAAGAGAACGCCTCCCCGAGTGAGCTG | |
| | CGAGACCTGCTGTCAGAGTTCAACGTCCTGAAGCAGGTCAACCACCCACATG | |
| | TCATCAAATTGTATGGGGCCTGCAGCCAGGATGGCCCGCTCCTCCTCATCGT | |
| | GGAGTACGCCAAATACGGCTCCCTGCGGGGCTTCCTCCGCGAGAGCCGCAAA | |
| | GTGGGGCCTGGCTACCTGGGCAGTGGAGGCAGCCGCAACTCCAGCTCCCTGG | |
| | ACCACCCGGATGAGCGGGCCCTCACCATGGGCGACCTCATCTCATTTGCCTG | |
| | GCAGATCTCACAGGGGATGCAGTATCTGGCCGAGATGAAGCTCGTTCATCGG | |
| | GACTTGGCAGCCAGAAACATCCTGGTAGCTGAGGGGCGGAAGATGAAGATTT | |
| | CGGATTTCGGCTTGTCCCGAGATGTTTATGAAGAGGATTCCTACGTGAAGAG | |
| | GAGCCAGGGTCGGATTCCAGTTAAATGGATGGCAATTGAATCCCTTTTTGAT | |
| | CATATCTACACCACGCAAAGTGATGTATGGTCTTTTGGTGTCCTGCTGTGGG | |
| | AGATCGTGACCCTAGGGGGAAACCCCTATCCTGGGATTCCTCCTGAGCGGCT | |
| | CTTCAACCTTCTGAAGACCGGCCACCGGATGGAGAGGCCAGACAACTGCAGC | |
| | GAGGAGATGTACCGCCTGATGCTGCAATGCTGGAAGCAGGAGCCGGACAAAA | |
| | GGCCGGTGTTTGCGGACATCAGCAAAGACCTGGAGAAGATGATGGTTAAGAG | |
| | GAGAGACTACTTGGACCTTGCGGCGTCCACTCCATCTGACTCCCTGATTTAT | |
| | GACGACGGCCTCTCAGAGGAGGAGACACCGCTGGTGGACTGTAATAATGCCC | |
| | CCCTCCCTCGAGCCCTCCCTTCCACATGGATTGAAAACAAACTCTATGGTAG | |
| | AATTTCCCATGCATTTACTAGATTCTAG | |
| ACPP- | ATGAGAGCTGCACCCCTCCTCCTGGCCAGGGCAGCAAGCCTTAGCCTTGGCT | 11 |
| RET | TCTTGTTTCTGCTTTTTTTCTGGCTAGACCGAAGTGTACTAGCCAAGGAGTT | |
| | GAAGTTTGTGACTTTGGTGTTTCGGCATGGAGACCGAAGTCCCATTGACACC | |
| | TTTCCCACTGACCCCATAAAGGAATCCTCATGGCCACAAGGATTTGGCCAAC | |
| | TCACCCAGCTGGGCATGGAGCAGCATTATGAACTTGGAGAGTATATAAGAAA | |
| | GAGATATAGAAAATTCTTGAATGAGTCCTATAAACATGAACAGGTTTATATT | |
| | CGAAGCACAGACGTTGACCGGACTTTGATGAGTGCTATGACAAACCTGGCAG | |
| | CCCTGTTTCCCCCAGAAGGTGTCAGCATCTGGAATCCTATCCTACTCTGGCA | |
| | GCCCATCCCGGTGCACACAGTTCCTCTTTCTGAAGATCAGTTGCTATACCTG | |
| | CCTTTCAGGAACTGCCCTCGTTTTCAAGAACTTGAGAGTGAGACTTTGAAAT | |
| | CAGAGGAATTCCAGAAGAGGCTGCACCCTTATAAGGATTTTATAGCTACCTT | |
| | GGGAAAACTTTCAGGATTACATGGCCAGGACCTTTTTGGAATTTGGAGTAAA | |
| | GTCTACGACCCTTTATATTGTGAGAGTGTTCACAATTTCACTTTACCCTCCT | |
| | GGGCCACTGAGGACACCATGACTAAGTTGAGAGAATTGTCAGAATTGTCCCT | |
| | CCTGTCCCTCTATGGAATTCACAAGCAGAAAGAGAAATCTAGGCTCCAAGGG | |
| | GGTGTCCTGGTCAATGAAATCCTCAATCACATGAAGAGAGCAACTCAGATAC | |
| | CAAGCTACAAAAAACTCATCATGTATTCTGCGCATGACACTACTGTGAGTGG | |
| | CCTACAGATGGCGCTAGATGTTTACAACGGACTCCTTCCTCCCTATGCTTCT | |
| | TGCCACTTGACGGAATTGTACTTTGAGAAGGGGGAGTACTTTGTGGAGATGT | |
| | ACTATCGGAATGAGACGCAGCACGAGCCGTATCCCCTCATGCTACCTGGCTG | |
| | CAGCCCCAGCTGTCCTCTGGAGAGGTTTGCTGAGCTGGTTGGCCCTGTGATC | |
| | CCTCAAGACTGGTCCACGGAGTGTATGACCACAAACAGCCATCAAGATCCAC | |
| | TGTGCGACGAGCTGTGCCGCACGGTGATCGCAGCCGCTGTCCTCTTCTCCTT | |
| | CATCGTCTCGGTGCTGCTGTCTGCCTTCTGCATCCACTGCTACCACAAGTTT | |
| | GCCCACAAGCCACCCATCTCCTCAGCTGAGATGACCTTCCGGAGGCCCGCCC | |
| | AGGCCTTCCCGGTCAGCTACTCCTCTTCCGGTGCCCGCCGGCCCTCGCTGGA | |
| | CTCCATGGAGAACCAGGTCTCCGTGGATGCCTTCAAGATCCTGGAGGATCCA | |
| | AAGTGGGAATTCCCTCGGAAGAACTTGGTTCTTGGAAAAACTCTAGGAGAAG | |
| | GCGAATTTGGAAAAGTGGTCAAGGCAACGGCCTTCCATCTGAAAGGCAGAGC | |
| | AGGGTACACCACGGTGGCCGTGAAGATGCTGAAAGAGAACGCCTCCCCGAGT | |
| | GAGCTGCGAGACCTGCTGTCAGAGTTCAACGTCCTGAAGCAGGTCAACCACC | |
| | CACATGTCATCAAATTGTATGGGGCCTGCAGCCAGGATGGCCCGCTCCTCCT | |
| | CATCGTGGAGTACGCCAAATACGGCTCCCTGCGGGGCTTCCTCCGCGAGAGC | |
| | CGCAAAGTGGGGCCTGGCTACCTGGGCAGTGGAGGCAGCCGCAACTCCAGCT | |
| | CCCTGGACCACCCGGATGAGCGGGCCCTCACCATGGGCGACCTCATCTCATT | |
| | TGCCTGGCAGATCTCACAGGGGATGCAGTATCTGGCCGAGATGAAGCTCGTT | |
| | CATCGGGACTTGGCAGCCAGAAACATCCTGGTAGCTGAGGGGCGGAAGATGA | |
| | AGATTTCGGATTTCGGCTTGTCCCGAGATGTTTATGAAGAGGATTCCTACGT | |
| | GAAGAGGAGCCAGGGTCGGATTCCAGTTAAATGGATGGCAATTGAATCCCTT | |
| | TTTGATCATATCTACACCACGCAAAGTGATGTATGGTCTTTTGGTGTCCTGC | |
| | TGTGGGAGATCGTGACCCTAGGGGGAAACCCCTATCCTGGGATTCCTCCTGA | |
| | GCGGCTCTTCAACCTTCTGAAGACCGGCCACCGGATGGAGAGGCCAGACAAC | |
| | TGCAGCGAGGAGATGTACCGCCTGATGCTGCAATGCTGGAAGCAGGAGCCGG | |
| | ACAAAAGGCCGGTGTTTGCGGACATCAGCAAAGACCTGGAGAAGATGATGGT | |
| | TAAGAGGAGAGACTACTTGGACCTTGCGGCGTCCACTCCATCTGACTCCCTG | |
| | ATTTATGACGACGGCCTCTCAGAGGAGGAGACACCGCTGGTGGACTGTAATA | |
| | ATGCCCCCCTCCCTCGAGCCCTCCCTTCCACATGGATTGAAAACAAACTCTA | |
| | TGGTAGAATTTCCCATGCATTTACTAGATTCTAG | |
| BMS1- | ATGGAGGCTAAGGACCAGAAGAAACACAGAAAGAAAAACAGTGGACCCAAAG | 12 |
| RET | CTGCAAAGAAAAAGAAGCGGCTTCTGCAGGATCTCCAGCTAGGAGACGAAGA | |
| | AGATGCCCGGAAGAGAAATCCCAAAGCTTTTGCAGTTCAGTCTGCTGTGCGG | |
| | ATGGCTCGATCCTTTCACAGGACTCAGGATTTGAAGACAAAAAAGCATCATA | |
| | TTCCAGTGGTTGATCGAACTCCACTAGAGCCCCCACCAATAGTGGTAGTGGT | |
| | GATGGGACCTCCAAAAGTTGGAAAGAGCACTTTGATACAATGCCTCATTCGG | |
| | AACTTTACCCGGCAGAAGTTGACTGAGATCAGAGGCCCTGTGACGATTGTGT | |
| | CAGGTAAAAAGCGCAGACTCACCATTATTGAATGTGGGTGTGACATTAACAT | |
| | GATGATTGATCTGGCTAAAGTAGCAGATCTGGTACTGATGCTTATAGATGCC | |
| | AGCTTTGGGTTTGAAATGGAAACGTTTGAGTTTCTAAACATCTGTCAAGTAC | |
| | ATGGCTTTCCTAAAATTATGGGAGTTCTCACCCACCTCGACTCCTTCAAGCA | |
| | TAATAAGCAACTGAAGAAGACAAAGAAGCGATTAAAACACAGGTTCTGGACG | |
| | GAAGTTTACCCGGGTGCCAAGCTGTTCTACCTTTCTGGAATGGTGCATGGAG | |
| | AATATCAAAACCAAGAAATCCACAATCTGGGCCGTTTTATTACAGTTATGAA | |
| | GTTTAGGCCTCTCACATGGCAAACTTCTCACCCTTATATCCTGGCAGACAGG | |
| | ATGGAAGATTTGACAAACCCAGAGGATATCCGAACAAACATCAAATGTGACC | |
| | GGAAGGTGTCACTTTATGGTTATTTAAGAGGAGCACACTTGAAAAATAAAAG | |
| | CCAAATTCACATGCCAGGGGTAGGAGATTTTGCCGTGAGTGACATCAGTTTC | |
| | CTCCCAGACCCTTGCGCTCTTCCTGAACAACAAAAGAAGCGCTGTTTAAATG | |
| | AGAAGGAGAAGCTGGTTTATGCGCCTCTTTCTGGAGTTGGGGGTGTGCTGTA | |
| | TGACAAAGACGCTGTCTATGTTGACCTTGGTGGCAGCCACGTTTTTCAGGAT | |
| | GAAGTGGGGCCCACCCATGAGCTGGTCCAGAGTCTCATCTCTACCCACTCCA | |
| | CCATTGATGCCAAGATGGCTTCAAGTCGAGTGACGCTGTTTTCTGATTCCAA | |
| | GCCACTTGGGTCAGAGGATATAGATAATCAAGGGCTAATGATGCCAAAGGAG | |
| | GAAAAACAAATGGACTTGAACACTGGTCGAATGCGTCGGAAAGCCATTTTCG | |
| | GAGATGAAGATGAATCTGGAGATAGTGATGATGAAGAAGATGATGAAATGTC | |
| | TGAAGATGACGGGTTGGAAAACGGCTCTAGTGATGAGGAAGCAGAAGAGGAG | |
| | GAAAATGCTGAGATGACTGATCAGTATATGGCTGTTAAGGGCATCAAACGAC | |
| | GGAAACTTGAGTTGGAAGAAGACAGTGAAATGGATTTGCCAGCATTTGCTGA | |
| | CAGTGACGATGACCTTGAGAGGAGCTCAGCGGAAGAAGGGGAAGCGGAGGAA | |
| | GCTGATGAAAGCAGTGAAGAAGAGGACTGCACTGCAGGAGAGAAGGGCATTT | |
| | CAGGATCAAAGGCTGCTGGAGAAGGTAGTAAAGCAGGGCTGTCACCAGCTAA | |
| | TTGCCAGAGTGACCGTGTGAATCTGGAGAAGTCTTTGCTGATGAAGAAAGCA | |
| | GCTCTCCCCACTTTCGATTCTGGGCATTGCACAGCTGAAGAGGTGTTTGCAT | |
| | CTGAAGATGAATCTGAAGAAAGCTCCTCACTCAGTGCAGAGGAAGAAGACTC | |
| | AGAAAATGAAGAGGCTATTAGAAAAAAGCTTTCAAAGCCTTCTCAAGTGAGC | |
| | AGTGGTCAGAAACTGGGGCCACAGAACTTCATTGATGAGACCAGTGATATAG | |
| | AAAATTTACTCAAAGAGGAAGAAGATTACAAGGAAGAAAATAATGATTCCAA | |
| | AGAAACGTCAGGTGCCCTCAAGTGGAAGGAAGACCTTTCCAGAAAGGCAGCT | |
| | GAGGCCTTTCTGAGGCAGCAGCAAGCAGCTCCAAACCTCCGAAAGCTTATTT | |
| | ATGGGACAGTGACAGAAGATAATGAAGAAGAAGATGATGATACTCTAGAAGA | |
| | GCTTGGAGGGTTGTTTCGTGTCAACCAGCCTGACAGAGAGTGTAAGCACAAG | |
| | GCTGACTCTTTGGACTGCTCCAGATTTCTTGTGGAGGCCCCCCATGACTGGG | |
| | ATTTAGAGGAGGTTATGAACAGTATCAGAGATTGCTTCGTGACTGGAAAGTG | |
| | GGAAGATGATAAAGATGCAGCCAAGGTCTTAGCAGAAGATGGGGGCAGCATT | |
| | GTTGGGGGACACGAGCCTGGGGAGCCCCGGGGGATTAAAGCTGGCTATGGCA | |
| | CCTGCAACTGCTTCCCTGAGGAGGAGAAGTGCTTCTGCGAGCCCGAAGACAT | |
| | CCAGGATCCACTGTGCGACGAGCTGTGCCGCACGGTGATCGCAGCCGCTGTC | |
| | CTCTTCTCCTTCATCGTCTCGGTGCTGCTGTCTGCCTTCTGCATCCACTGCT | |
| | ACCACAAGTTTGCCCACAAGCCACCCATCTCCTCAGCTGAGATGACCTTCCG | |
| | GAGGCCCGCCCAGGCCTTCCCGGTCAGCTACTCCTCTTCCGGTGCCCGCCGG | |
| | CCCTCGCTGGACTCCATGGAGAACCAGGTCTCCGTGGATGCCTTCAAGATCC | |
| | TGGAGGATCCAAAGTGGGAATTCCCTCGGAAGAACTTGGTTCTTGGAAAAAC | |
| | TCTAGGAGAAGGCGAATTTGGAAAAGTGGTCAAGGCAACGGCCTTCCATCTG | |
| | AAAGGCAGAGCAGGGTACACCACGGTGGCCGTGAAGATGCTGAAAGAGAACG | |
| | CCTCCCCGAGTGAGCTGCGAGACCTGCTGTCAGAGTTCAACGTCCTGAAGCA | |
| | GGTCAACCACCCACATGTCATCAAATTGTATGGGGCCTGCAGCCAGGATGGC | |
| | CCGCTCCTCCTCATCGTGGAGTACGCCAAATACGGCTCCCTGCGGGGCTTCC | |
| | TCCGCGAGAGCCGCAAAGTGGGGCCTGGCTACCTGGGCAGTGGAGGCAGCCG | |
| | CAACTCCAGCTCCCTGGACCACCCGGATGAGCGGGCCCTCACCATGGGCGAC | |
| | CTCATCTCATTTGCCTGGCAGATCTCACAGGGGATGCAGTATCTGGCCGAGA | |
| | TGAAGCTCGTTCATCGGGACTTGGCAGCCAGAAACATCCTGGTAGCTGAGGG | |
| | GCGGAAGATGAAGATTTCGGATTTCGGCTTGTCCCGAGATGTTTATGAAGAG | |
| | GATTCCTACGTGAAGAGGAGCCAGGGTCGGATTCCAGTTAAATGGATGGCAA | |
| | TTGAATCCCTTTTTGATCATATCTACACCACGCAAAGTGATGTATGGTCTTT | |
| | TGGTGTCCTGCTGTGGGAGATCGTGACCCTAGGGGGAAACCCCTATCCTGGG | |
| | ATTCCTCCTGAGCGGCTCTTCAACCTTCTGAAGACCGGCCACCGGATGGAGA | |
| | GGCCAGACAACTGCAGCGAGGAGATGTACCGCCTGATGCTGCAATGCTGGAA | |
| | GCAGGAGCCGGACAAAAGGCCGGTGTTTGCGGACATCAGCAAAGACCTGGAG | |
| | AAGATGATGGTTAAGAGGAGAGACTACTTGGACCTTGCGGCGTCCACTCCAT | |
| | CTGACTCCCTGATTTATGACGACGGCCTCTCAGAGGAGGAGACACCGCTGGT | |
| | GGACTGTAATAATGCCCCCCTCCCTCGAGCCCTCCCTTCCACATGGATTGAA | |
| | AACAAACTCTATGGTAGAATTTCCCATGCATTTACTAGATTCTAG | |
| CEP135- | ATGACTACAGCTGTAGAGAGAAAGTATATTAATATTAGGAAAAGGCTGGATC | 13 |
| RET | AGCTGGGATACCGCCAGACTCTGACAGTGGAGTGTTTACCTTTGGTAGAAAA | |
| | ACTTTTCAGCGACTTAGTTCATACAACTGAGAGCCTTCGGCAATCAAAATTA | |
| | TCTGCTGTGAAAGCTGAAAAAGAAAGTGCCAATTTTGATTTTGTTTTGGAAC | |
| | CCTATAAACTTGAAAATGCAAGATTGAGTAGAGAAAATAATGAATTATACCT | |
| | AGAGTTAATGAAACTGAGAGAACATTCAGACCAACACGTTAAAGAGTTGAAA | |
| | ACTTCATTGAAGAAATGTGCACGTGAAACAGCTGATCTGAAATTTCTGAATA | |
| | ACCAATATGCTCATAAACTCAAACTGTTGGAGAAAGAGAGCAAAGCTAAGAA | |
| | TGAAAGAATTCAACAACTTCAAGAAAAGAATTTGCATGCTGTAGTACAAACT | |
| | CCAGGTGGCAAGAAAAGAAGTATTGCTTTCAGGCGCCAGCGTATGCAAATTG | |
| | ATGAACCGGTTCCTCCCTCTGAAGTCAGTTCATATCCAGTTCCTCAACCAGA | |
| | TGACCCTTACATTGCAGACCTCCTTCAAGTGGCTGATAACAGGATTCAAGAA | |
| | CTTCAACAGGAAGTCCACCAGCTACAAGAAAAGTTAGCAATGATGGAAAGTG | |
| | GGGTGAGAGACTATAGCAAGCAGATTGAGCTAAGAGAACGAGAGATAGAACG | |
| | ACTGTCAGTTGCTTTGGATGGTGGTCGGTCCCCTGATGTCCTTTCTCTGGAG | |
| | TCTAGAAATAAAACCAATGAAAAGCTTATTGCTCATTTAAATATTCAGGTTG | |
| | ACTTTCTTCAGCAAGCTAATAAAGACCTGGAGAAGCGTATACGAGAGCTTAT | |
| | GGAAACCAAGGAAACAGTGACATCTGAAGTCGTTAATTTAAGTAACAAAAAT | |
| | GAAAAACTCTGCCAAGAATTAACTGAAATAGATCAGTTAGCACAGCAGTTGG | |
| | AAAGACATAAAGAAGAAGTGCTTGAGACTGCTGATAAAGAGCTTGGGGAAGC | |
| | AAAGAAAGAGATTAAAAGAAAGCTCTCTGAAATGCAGGATCTTGAAGAAACA | |
| | ATGGCAAAACTTCAGCTGGAATTGAACTTATGCCAGAAAGAAAAGGAGAGAC | |
| | TGAGTGATGAACTCCTTGTAAAATCAGACCTAGAAACTGTTGTTCATCAGCT | |
| | TGAACAAGAAAAGCAAAGACTTAGCAAAAAAGTTGAAAGTTTTGCAGTTACA | |
| | GAACGACAACTTACTCTGGAGGTTGAGAGGATGAGACTAGAACATGGAATAA | |
| | AACGTCGAGACAGGTCACCTTCTCGTTTAGATACATTTCTGAAAGGTATAGA | |
| | AGAAGAACGAGATTATTATAAGAAAGAGCTAGAGAGACTCCAACATATAATA | |
| | CAGCGAAGATCTTGCTCTACAAGTTATAGCGCACGTGAAAAAAGTTCAATAT | |
| | TTAGAACACCAGAAAAGGAGGATCCAAAGTGGGAATTCCCTCGGAAGAACTT | |
| | GGTTCTTGGAAAAACTCTAGGAGAAGGCGAATTTGGAAAAGTGGTCAAGGCA | |
| | ACGGCCTTCCATCTGAAAGGCAGAGCAGGGTACACCACGGTGGCCGTGAAGA | |
| | TGCTGAAAGAGAACGCCTCCCCGAGTGAGCTGCGAGACCTGCTGTCAGAGTT | |
| | CAACGTCCTGAAGCAGGTCAACCACCCACATGTCATCAAATTGTATGGGGCC | |
| | TGCAGCCAGGATGGCCCGCTCCTCCTCATCGTGGAGTACGCCAAATACGGCT | |
| | CCCTGCGGGGCTTCCTCCGCGAGAGCCGCAAAGTGGGGCCTGGCTACCTGGG | |
| | CAGTGGAGGCAGCCGCAACTCCAGCTCCCTGGACCACCCGGATGAGCGGGCC | |
| | CTCACCATGGGCGACCTCATCTCATTTGCCTGGCAGATCTCACAGGGGATGC | |
| | AGTATCTGGCCGAGATGAAGCTCGTTCATCGGGACTTGGCAGCCAGAAACAT | |
| | CCTGGTAGCTGAGGGGCGGAAGATGAAGATTTCGGATTTCGGCTTGTCCCGA | |
| | GATGTTTATGAAGAGGATTCCTACGTGAAGAGGAGCCAGGGTCGGATTCCAG | |
| | TTAAATGGATGGCAATTGAATCCCTTTTTGATCATATCTACACCACGCAAAG | |
| | TGATGTATGGTCTTTTGGTGTCCTGCTGTGGGAGATCGTGACCCTAGGGGGA | |
| | AACCCCTATCCTGGGATTCCTCCTGAGCGGCTCTTCAACCTTCTGAAGACCG | |
| | GCCACCGGATGGAGAGGCCAGACAACTGCAGCGAGGAGATGTACCGCCTGAT | |
| | GCTGCAATGCTGGAAGCAGGAGCCGGACAAAAGGCCGGTGTTTGCGGACATC | |
| | AGCAAAGACCTGGAGAAGATGATGGTTAAGAGGAGAGACTACTTGGACCTTG | |
| | CGGCGTCCACTCCATCTGACTCCCTGATTTATGACGACGGCCTCTCAGAGGA | |
| | GGAGACACCGCTGGTGGACTGTAATAATGCCCCCCTCCCTCGAGCCCTCCCT | |
| | TCCACATGGATTGAAAACAAACTCTATGGTAGAATTTCCCATGCATTTACTA | |
| | GATTCTAG | |
| EEA1- | ATGTTAAGGAGGATTTTACAGAGGACTCCTGGGAGAGTTGGCTCTCAAGGTT | 14 |
| RET | CTGATTTAGATTCATCAGCAACTCCTATAAACACAGTGGACGTCAATAATGA | |
| | AAGCTCTTCAGAGGGTTTCATATGTCCCCAGTGTATGAAATCTCTTGGATCT | |
| | GCTGATGAACTTTTCAAACATTATGAAGCTGTTCATGATGCTGGTAATGACT | |
| | CAGGTCATGGAGGAGAGTCTAATCTTGCTTTGAAGCGAGATGATGTAACACT | |
| | GCTCAGACAAGAGGTCCAAGACCTACAGGCTTCACTTAAGGAAGAAAAATGG | |
| | TACTCGGAAGAATTAAAGAAGGAATTAGAAAAATATCAAGGGCTGCAGCAGC | |
| | AAGAGGCCAAACCTGATGGGTTGGTGACTGATTCATCAGCAGAACTACAGTC | |
| | TTTGGAACAGCAATTAGAAGAAGCCCAAACAGAAAATTTTAATATTAAGCAA | |
| | ATGAAAGACTTATTTGAACAGAAAGCAGCCCAACTTGCTACTGAAATTGCAG | |
| | ATATAAAGTCAAAGTATGATGAAGAAAGGAGTCTTCGAGAAGCTGCTGAACA | |
| | AAAAGTGACACGTCTGACAGAAGAATTAAACAAAGAGGCAACTGTAATTCAA | |
| | GATCTGAAGACGGAACTGCTTCAGAGACCTGGTATAGAAGATGTTGCCGTGC | |
| | TAAAGAAAGAACTGGTCCAAGTTCAAACACTAATGGATAACATGACCTTGGA | |
| | ACGTGAGCGAGAATCTGAAAAACTCAAAGATGAATGCAAAAAATTGCAGTCA | |
| | CAATATGCTAGCTCAGAGGCCACAATAAGCCAGCTAAGGAGTGAACTTGCCA | |
| | AAGGCCCCCAGGAAGTTGCTGTATATGTACAGGAACTACAAAAACTGAAAAG | |
| | TTCAGTTAATGAATTAACACAAAAAAATCAGACCTTGACAGAAAACTTGCTG | |
| | AAAAAAGAACAAGACTATACTAAGTTAGAGGAGAAACATAATGAAGAATCTG | |
| | TGAGTAAAAAGAATATTCAGGCAACCCTTCATCAAAAAGACCTAGATTGTCA | |
| | ACAGCTTCAGTCAAGATTGTCTGCATCTGAAACCTCACTGCATAGAATACAT | |
| | GTAGAACTAAGTGAAAAAGGAGAAGCTACTCAAAAGCTCAAAGAAGAATTAT | |
| | CTGAGGTAGAGACCAAGTACCAGCATCTAAAGGCGGAGTTTAAGCAGCTACA | |
| | ACAACAGAGAGAAGAAAAGGAGCAGCATGGGTTACAACTCCAAAGTGAAATT | |
| | AATCAATTACATAGCAAACTTCTGGAGACAGAGCGCCAACTAGGGGAAGCTC | |
| | ATGGTAGGCTGAAGGAACAGAGACAGCTTTCAAGTGAAAAGTTGATGGATAA | |
| | AGAACAACAAGTGGCTGATTTACAACTCAAACTTTCTCGGTTAGAAGAGCAG | |
| | TTGAAGGAAAAAGTTACAAATTCTACAGAATTGCAGCATCAATTAGATAAAA | |
| | CAAAGCAACAGCATCAAGAACAACAGGCTCTTCAGCAAAGCACCACGGCAAA | |
| | ACTTCGAGAAGCTCAGGAGGATCCAAAGTGGGAATTCCCTCGGAAGAACTTG | |
| | GTTCTTGGAAAAACTCTAGGAGAAGGCGAATTTGGAAAAGTGGTCAAGGCAA | |
| | CGGCCTTCCATCTGAAAGGCAGAGCAGGGTACACCACGGTGGCCGTGAAGAT | |
| | GCTGAAAGAGAACGCCTCCCCGAGTGAGCTGCGAGACCTGCTGTCAGAGTTC | |
| | AACGTCCTGAAGCAGGTCAACCACCCACATGTCATCAAATTGTATGGGGCCT | |
| | GCAGCCAGGATGGCCCGCTCCTCCTCATCGTGGAGTACGCCAAATACGGCTC | |
| | CCTGCGGGGCTTCCTCCGCGAGAGCCGCAAAGTGGGGCCTGGCTACCTGGGC | |
| | AGTGGAGGCAGCCGCAACTCCAGCTCCCTGGACCACCCGGATGAGCGGGCCC | |
| | TCACCATGGGCGACCTCATCTCATTTGCCTGGCAGATCTCACAGGGGATGCA | |
| | GTATCTGGCCGAGATGAAGCTCGTTCATCGGGACTTGGCAGCCAGAAACATC | |
| | CTGGTAGCTGAGGGGCGGAAGATGAAGATTTCGGATTTCGGCTTGTCCCGAG | |
| | ATGTTTATGAAGAGGATTCCTACGTGAAGAGGAGCCAGGGTCGGATTCCAGT | |
| | TAAATGGATGGCAATTGAATCCCTTTTTGATCATATCTACACCACGCAAAGT | |
| | GATGTATGGTCTTTTGGTGTCCTGCTGTGGGAGATCGTGACCCTAGGGGGAA | |
| | ACCCCTATCCTGGGATTCCTCCTGAGCGGCTCTTCAACCTTCTGAAGACCGG | |
| | CCACCGGATGGAGAGGCCAGACAACTGCAGCGAGGAGATGTACCGCCTGATG | |
| | CTGCAATGCTGGAAGCAGGAGCCGGACAAAAGGCCGGTGTTTGCGGACATCA | |
| | GCAAAGACCTGGAGAAGATGATGGTTAAGAGGAGAGACTACTTGGACCTTGC | |
| | GGCGTCCACTCCATCTGACTCCCTGATTTATGACGACGGCCTCTCAGAGGAG | |
| | GAGACACCGCTGGTGGACTGTAATAATGCCCCCCTCCCTCGAGCCCTCCCTT | |
| | CCACATGGATTGAAAACAAACTCTATGGTAGAATTTCCCATGCATTTACTAG | |
| | ATTCTAG | |
| CSGALNA | ATGCCTAGAAGAGGACTGATTCTTCACACCCGGACCCACTGGTTGCTGTTGG | 15 |
| CT2-RET | GCCTTGCTTTGCTCTGCAGTTTGGTATTATTTATGTACCTCCTGGAATGTGC | |
| | CCCCCAGACTGATGGAAATGCATCTCTTCCTGGTGTTGTTGGGGAAAATTAT | |
| | GGTAAAGAGTATTATCAAGCCCTCCTACAGGAACAAGAAGAACATTATCAGA | |
| | CCAGGGCAACCAGTCTGAAACGCCAAATTGCCCAACTAAAACAAGAATTACA | |
| | AGAAATGAGTGAGAAGATGCGGTCACTGCAAGAAAGAAGGAATGTAGGGGCT | |
| | AATGGCATAGGCTATCAGAGCAACAAAGAGCAAGCACCTAGTGATCTTTTAG | |
| | AGTTTCTTCATTCCCAAATTGACAAAGCTGAAGTTAGCATAGGGGCCAAACT | |
| | ACCCAGTGAGTATGGGGTCATTCCCTTTGAAAGTTTTACCTTAATGAAAGTA | |
| | TTTCAATTGGAAATGGGTCTCACTCGCCATCCTGAAGAAAAGCCAGTTAGAA | |
| | AAGACAAACGAGATGAATTGGTGGAAGTTATTGAAGCGGGCTTGGAGGTCAT | |
| | TAATAATCCTGATGAAGATGATGAACAAGAAGATGAGGAGGGTCCCCTTGGA | |
| | GAGAAACTGATATTTAATGAAAATGACTTCGTAGAAGGTTATTATCGCACTG | |
| | AGAGAGATAAGGGCACACAGTATGAACTCTTTTTTAAGAAAGCAGACCTTAC | |
| | GGAATATAGACATGTGACCCTCTTCCGCCCTTTTGGACCTCTCATGAAAGTG | |
| | AAGAGTGAGATGATTGACATCACTAGATCAATTATTAATATCATTGTGCCAC | |
| | TTGCTGAAAGAACTGAAGCATTTGTACAATTTATGCAGAACTTCAGGGATGT | |
| | TTGTATTCATCAAGACAAGAAGATTCATCTCACAGTGGTGTATTTTGGTAAA | |
| | GAAGGACTGTCTAAAGTCAAGTCTATCCTAGAATCTGTCACCAGTGAGTCTA | |
| | ATTTTCACAATTACACCTTGGTCTCATTGAATGAAGAATTTAATCGTGGACG | |
| | AGGACTAAATGTGGGTGCCCGAGCTTGGGACAAGGGAGAGGTCTTGATGTTT | |
| | TTCTGTGATGTTGATATCTATTTCTCAGCCGAATTCCTTAACAGCTGCCGGT | |
| | TAAATGCTGAGCCAGGTAAGAAGGTGTTTTACCCTGTGGTGTTCAGTCTTTA | |
| | CAATCCTGCCATTGTTTATGCCAACCAGGAAGTGCCACCACCTGTGGAGCAG | |
| | CAGCTGGAGGATCCAAAGTGGGAATTCCCTCGGAAGAACTTGGTTCTTGGAA | |
| | AAACTCTAGGAGAAGGCGAATTTGGAAAAGTGGTCAAGGCAACGGCCTTCCA | |
| | TCTGAAAGGCAGAGCAGGGTACACCACGGTGGCCGTGAAGATGCTGAAAGAG | |
| | AACGCCTCCCCGAGTGAGCTGCGAGACCTGCTGTCAGAGTTCAACGTCCTGA | |
| | AGCAGGTCAACCACCCACATGTCATCAAATTGTATGGGGCCTGCAGCCAGGA | |
| | TGGCCCGCTCCTCCTCATCGTGGAGTACGCCAAATACGGCTCCCTGCGGGGC | |
| | TTCCTCCGCGAGAGCCGCAAAGTGGGGCCTGGCTACCTGGGCAGTGGAGGCA | |
| | GCCGCAACTCCAGCTCCCTGGACCACCCGGATGAGCGGGCCCTCACCATGGG | |
| | CGACCTCATCTCATTTGCCTGGCAGATCTCACAGGGGATGCAGTATCTGGCC | |
| | GAGATGAAGCTCGTTCATCGGGACTTGGCAGCCAGAAACATCCTGGTAGCTG | |
| | AGGGGCGGAAGATGAAGATTTCGGATTTCGGCTTGTCCCGAGATGTTTATGA | |
| | AGAGGATTCCTACGTGAAGAGGAGCCAGGGTCGGATTCCAGTTAAATGGATG | |
| | GCAATTGAATCCCTTTTTGATCATATCTACACCACGCAAAGTGATGTATGGT | |
| | CTTTTGGTGTCCTGCTGTGGGAGATCGTGACCCTAGGGGGAAACCCCTATCC | |
| | TGGGATTCCTCCTGAGCGGCTCTTCAACCTTCTGAAGACCGGCCACCGGATG | |
| | GAGAGGCCAGACAACTGCAGCGAGGAGATGTACCGCCTGATGCTGCAATGCT | |
| | GGAAGCAGGAGCCGGACAAAAGGCCGGTGTTTGCGGACATCAGCAAAGACCT | |
| | GGAGAAGATGATGGTTAAGAGGAGAGACTACTTGGACCTTGCGGCGTCCACT | |
| | CCATCTGACTCCCTGATTTATGACGACGGCCTCTCAGAGGAGGAGACACCGC | |
| | TGGTGGACTGTAATAATGCCCCCCTCCCTCGAGCCCTCCCTTCCACATGGAT | |
| | TGAAAACAAACTCTATGGTAGAATTTCCCATGCATTTACTAGATTCTAG | |
| KIAA1217- | ATGGAAGAAAATGAAAGCCAGAAATGTGAGCCGTGCCTTCCTTACTCAGCAG | 16 |
| RET | ACAGAAGACAGATGCAGGAACAAGGCAAAGGCAATCTGCATGTAACATCACC | |
| | AGAAGATGCAGAATGCCGCAGAACCAAGGAACGCCTTTCTAATGGAAACAGT | |
| | CGTGGTTCAGTTTCCAAGTCTTCCCGCAATATCCCAAGGAGACACACCCTAG | |
| | GGGGGCCCCGAAGTTCCAAGGAAATACTGGGAATGCAAACATCTGAGATGGA | |
| | TCGGAAGAGAGAAGCGTTCCTAGAACATCTGAAGCAGAAGTACCCCCACCAC | |
| | GCCTCTGCAATCATGGGTCACCAAGAGAGGCTGAGAGACCAGACAAGGAGCC | |
| | CCAAACTGTCTCACAGTCCTCAACCACCCAGTCTGGGTGACCCGGTCGAGCA | |
| | TTTATCAGAGACGTCCGCTGATTCTTTGGAAGCCATGTCTGAGGGGGATGCT | |
| | CCAACCCCTTTTTCCAGAGGCAGCCGGACTCGTGCGAGCCTTCCTGTGGTGA | |
| | GGTCAACCAACCAGACGAAAGAAAGATCTCTGGGGGTTCTCTATCTCCAGTA | |
| | TGGAGATGAAACCAAGCAGCTCAGGATGCCGAATGAAATCACAAGTGCAGAC | |
| | ACAATCCGTGCTCTCTTCGTAAGTGCCTTTCCACAGCAGCTCACCATGAAAA | |
| | TGCTGGAATCGCCCAGTGTCGCCATTTACATCAAAGATGAAAGCAGAAATGT | |
| | CTATTATGAATTAAATGATGTAAGGAACATTCAAGACAGATCACTCCTCAAA | |
| | GTGTACAACAAGGATCCTGCACATGCGTTTAATCACACACCAAAAACTATGA | |
| | ATGGAGACATGAGGATGCAGAGAGAACTTGTTTATGCAAGAGGAGATGGCCC | |
| | TGGGGCCCCTCGCCCCGGATCTACTGCTCATCCACCCCATGCGATTCCAAAT | |
| | TCCCCACCGTCTACTCCAGTGCCCCATTCCATGCCCCCCTCCCCGTCCAGAA | |
| | TTCCTTATGGGGGCACCCGCTCCATGGTTGTTCCTGGCAATGCCACCATCCC | |
| | CAGGGACAGAATCTCCAGCCTGCCAGTCTCCAGACCCATCTCTCCAAGCCCA | |
| | AGCGCCATTTTAGAAAGAAGAGATGTCAAGCCTGATGAAGACATGAGTGGCA | |
| | AAAACATTGCAATGTACAGAAATGAGGGTTTCTATGCTGATCCTTACCTTTA | |
| | TCACGAGGGACGGATGAGCATAGCCTCATCCCATGGTGGACACCCACTGGAT | |
| | GTCCCCGACCACATCATTGCATATCACCGCACCGCCATCCGGTCAGCGAGTG | |
| | CTTATTGTAACCCCTCAATGCAAGCGGAAATGCATATGGAACAATCACTGTA | |
| | CAGACAGAAATCAAGGAAATATCCGGATAGCCATTTGCCTACACTGGGCTCC | |
| | AAAACACCCCCTGCCTCTCCTCACAGAGTCAGTGACCTGAGGATGATAGACA | |
| | TGCACGCTCACTATAATGCCCACGGCCCCCCTCACACCATGCAGCCAGACCG | |
| | GGCCTCTCCGAGCCGCCAGGCCTTTAAAAAGGAGCCAGGCACCTTGGTGTAT | |
| | ATAGAAAAGCCACGGAGCGCTGCAGGATTATCCAGCCTTGTAGACCTCGGCC | |
| | CTCCTCTAATGGAGAAGCAAGTTTTTGCCTACAGCACGGCGACAATACCCAA | |
| | AGACAGAGAGACCAGAGAGAGGATGCAAGCCATGGAGAAACAGATTGCCAGT | |
| | TTAACTGGCCTTGTTCAGTCTGCGCTTTTTAAAGGGCCCATTACAAGTTATA | |
| | GCAAAGATGCGTCTAGCGAGAAAATGATGAAAACCACAGCCAACAGGAACCA | |
| | CACAGATAGTGCAGGAACGCCCCATGTGTCTGGTGGGAAGATGCTCAGTGCT | |
| | CTGGAGTCCACGGTGCCTCCCAGCCAGCCTCCACCTGTGGGCACCTCAGCCA | |
| | TCCACATGAGCCTGCTTGAGATGAGGCGGAGCGTGGCGGAACTCAGGCTCCA | |
| | GCTCCAGCAGATGCGGCAGCTCCAGCTGCAGAACCAGGAGTTGCTGAGGGCA | |
| | ATGATGAAGAAGGCCGAGCTGGAAATCAGTGGCAAAGTGATGGAAACAATGA | |
| | AGAGACTGGAGGATCCCGTGCAGCGACAGCGCGTCCTAGTGGAGCAAGAGAG | |
| | ACAAAAATATCTTCATGAGGAAGAGAAGATCGTCAAGAAGTTGTGCGAGTTG | |
| | GAAGACTTTGTTGAAGACTTGAAGAAGGACTCCACGGCAGCCAGCCGATTGG | |
| | TTACTCTGAAAGACGTGGAAGACGGGGCTTTCCTCCTGCGTCAAGTGGGAGA | |
| | GGCTGTAGCTACCCTGAAAGATGTGGCCGAGGAGGCGGGCTGCCCCCTGTCC | |
| | TGTGCAGTCAGCAAGAGACGGCTGGAGTGTGAGGAGTGTGGCGGCCTGGGCT | |
| | CCCCAACAGGCAGGTGTGAGTGGAGGCAAGGAGATGGCAAAGGGATCACCAG | |
| | GAACTTCTCCACCTGCTCTCCCAGCACCAAGACCTGCCCCGACGGCCACTGC | |
| | GATGTTGTGGAGACCCAAGACATCAACATTTGCCCTCAGGACTGCCTCCGGG | |
| | GCAGCATTGTTGGGGGACACGAGCCTGGGGAGCCCCGGGGGATTAAAGCTGG | |
| | CTATGGCACCTGCAACTGCTTCCCTGAGGAGGAGAAGTGCTTCTGCGAGCCC | |
| | GAAGACATCCAGGATCCACTGTGCGACGAGCTGTGCCGCACGGTGATCGCAG | |
| | CCGCTGTCCTCTTCTCCTTCATCGTCTCGGTGCTGCTGTCTGCCTTCTGCAT | |
| | CCACTGCTACCACAAGTTTGCCCACAAGCCACCCATCTCCTCAGCTGAGATG | |
| | ACCTTCCGGAGGCCCGCCCAGGCCTTCCCGGTCAGCTACTCCTCTTCCGGTG | |
| | CCCGCCGGCCCTCGCTGGACTCCATGGAGAACCAGGTCTCCGTGGATGCCTT | |
| | CAAGATCCTGGAGGATCCAAAGTGGGAATTCCCTCGGAAGAACTTGGTTCTT | |
| | GGAAAAACTCTAGGAGAAGGCGAATTTGGAAAAGTGGTCAAGGCAACGGCCT | |
| | TCCATCTGAAAGGCAGAGCAGGGTACACCACGGTGGCCGTGAAGATGCTGAA | |
| | AGAGAACGCCTCCCCGAGTGAGCTGCGAGACCTGCTGTCAGAGTTCAACGTC | |
| | CTGAAGCAGGTCAACCACCCACATGTCATCAAATTGTATGGGGCCTGCAGCC | |
| | AGGATGGCCCGCTCCTCCTCATCGTGGAGTACGCCAAATACGGCTCCCTGCG | |
| | GGGCTTCCTCCGCGAGAGCCGCAAAGTGGGGCCTGGCTACCTGGGCAGTGGA | |
| | GGCAGCCGCAACTCCAGCTCCCTGGACCACCCGGATGAGCGGGCCCTCACCA | |
| | TGGGCGACCTCATCTCATTTGCCTGGCAGATCTCACAGGGGATGCAGTATCT | |
| | GGCCGAGATGAAGCTCGTTCATCGGGACTTGGCAGCCAGAAACATCCTGGTA | |
| | GCTGAGGGGCGGAAGATGAAGATTTCGGATTTCGGCTTGTCCCGAGATGTTT | |
| | ATGAAGAGGATTCCTACGTGAAGAGGAGCCAGGGTCGGATTCCAGTTAAATG | |
| | GATGGCAATTGAATCCCTTTTTGATCATATCTACACCACGCAAAGTGATGTA | |
| | TGGTCTTTTGGTGTCCTGCTGTGGGAGATCGTGACCCTAGGGGGAAACCCCT | |
| | ATCCTGGGATTCCTCCTGAGCGGCTCTTCAACCTTCTGAAGACCGGCCACCG | |
| | GATGGAGAGGCCAGACAACTGCAGCGAGGAGATGTACCGCCTGATGCTGCAA | |
| | TGCTGGAAGCAGGAGCCGGACAAAAGGCCGGTGTTTGCGGACATCAGCAAAG | |
| | ACCTGGAGAAGATGATGGTTAAGAGGAGAGACTACTTGGACCTTGCGGCGTC | |
| | CACTCCATCTGACTCCCTGATTTATGACGACGGCCTCTCAGAGGAGGAGACA | |
| | CCGCTGGTGGACTGTAATAATGCCCCCCTCCCTCGAGCCCTCCCTTCCACAT | |
| | GGATTGAAAACAAACTCTATGGTAGAATTTCCCATGCATTTACTAGATTCTA | |
| | G | |
| SORBS1- | ATGAGTTCTGAATGTGATGGTGGTTCCAAAGCTGTGATGAATGGCTTGGCAC | 17 |
| RET | CTGGCAGCAATGGGCAAGACAAAGACATGGATCTTACAAAAATCTGCACTGG | |
| | GAAGGGAGCGGTGACTCTCCGGGCCTCGTCTTCCTACAGGGAAACCCCAAGC | |
| | AGTAGCCCTGCGAGCCCTCAGGAAACCCGGCAACACGAAAGCAAACCAGATG | |
| | AGTGGAGGCTTTCTTCCAGTGCTGATGCCAATGGAAATGCCCAGCCCTCTTC | |
| | ACTCGCTGCCAAGGGCTACAGAAGTGTGCATCCCAACCTTCCTTCTGACAAG | |
| | TCCCAGGATTCCAGTCCTCTACTAAATGAAGTTTCTTCTTCCCTTATTGGAA | |
| | CTGATTCCCAAGCCTTTCCATCAGTTAGCAAGCCTTCATCCGCCTATCCCTC | |
| | CACAACGATTGTCAATCCTACTATTGTGCTCTTGCAACACAATCGAGAACAG | |
| | CAAAAACGACTCAGTAGCCTTTCAGATCCTGTCTCAGAAAGAAGAGTGGGAG | |
| | AGCAGGACTCAGCACCAACCCAGGAAAAACCCACCTCACCTGGCAAGGCTAT | |
| | TGAAAAAAGAGCAAAGGATGACAGTAGGCGGGTGGTGAAGAGCACTCAGGAC | |
| | TTAAGCGATGTTTCCATGGATGAAGTGGGCATCCCACTCCGGAACACTGAGA | |
| | GATCAAAAGACTGGTACAAGACTATGTTTAAACAGATCCACAAACTGAACAG | |
| | AGATGATGATTCAGATCTGTACTCTCCCAGATACTCATTTTCTGAAGACACA | |
| | AAATCTCCCCTTTCTGTGCCTCGCTCAAAAAGTGAGATGAGCTACATTGATG | |
| | GTGAGAAGGTAGTCAAGAGGTCGGCCACACTACCCCTCCCAGCCCGCTCTTC | |
| | CTCACTGAAGTCAAGCTCAGAAAGAAATGACTGGGAACCCCCAGATAAGAAA | |
| | GTAGACACAAGAAAATATCGTGCAGAGCCCAAGAGCATTTACGAATATCAGC | |
| | CTGGCAAGTCTTCCGTTCTGACCAACGAAAAGATGAGCTCAGCCATCAGCCC | |
| | TACTCCGGAAATTTCTTCAGAGACTCCTGGATATATATATTCTTCCAACTTC | |
| | CATGCAGTGAAGAGGGAATCAGACGGGGCTCCTGGGGATCTCACTAGCTTGG | |
| | AGAATGAGAGACAAATTTATAAAAGTGTCTTGGAAGGTGGTGACATCCCTCT | |
| | TCAGGGCCTGAGTGGGCTCAAGCGACCATCCAGCTCTGCTTCCACTAAAGAT | |
| | TCAGAATCGCCAAGACATTTTATACCAGCTGATTACTTGGAATCCACGGAAG | |
| | AATTTATTCGAAGACGTCATGATGATAAAGAGATGAGACCTGCCAGAGCCAA | |
| | ATTTGACTTTAAAGCTCAGACACTAAAGGAGCTTCCTCTGCAGAAGGGAGAT | |
| | ATTGTTTACATTTATAAGCAAATTGATCAGAACTGGTATGAAGGAGAACACC | |
| | ACGGCCGGGTGGGAATCTTCCCACGCACCTACATCGAGCTTCTTCCTCCTGC | |
| | TGAGAAGGCACAGCCCAAAAAGTTGACACCAGTGCAGGTTTTGGAATATGGA | |
| | GAAGCTATTGCTAAGTTTAACTTTAATGGTGATACACAAGTAGAAATGTCCT | |
| | TCAGAAAGGGTGAGAGGATCACACTGCTCCGGCAGGTAGATGAGAACTGGTA | |
| | CGAAGGGAGGATCCCGGGGACATCCCGACAAGGCATCTTCCCCATCACCTAC | |
| | GTGGATGTGATCAAGCGACCACTGGTGAAAAACCCTGTGGATTACATGGACC | |
| | TGCCTTTCTCCTCCTCCCCAAGTCGCAGTGCCACTGCAAGCCCACAGATCGG | |
| | GAAAGTCTGTGTGGAAAACTGCCAGGCATTCAGTGGCATCAACGTCCAGTAC | |
| | AAGCTGCATTCCTCTGGTGCCAACTGCAGCACGCTAGGGGTGGTCACCTCAG | |
| | CCGAGGACACCTCGGGGATCCTGTTTGTGAATGACACCAAGGCCCTGCGGCG | |
| | GCCCAAGTGTGCCGAACTTCACTACATGGTGGTGGCCACCGACCAGCAGACC | |
| | TCTAGGCAGGCCCAGGCCCAGCTGCTTGTAACAGTGGAGGGGTCATATGTGG | |
| | CCGAGGAGGCGGGCTGCCCCCTGTCCTGTGCAGTCAGCAAGAGACGGCTGGA | |
| | GTGTGAGGAGTGTGGCGGCCTGGGCTCCCCAACAGGCAGGTGTGAGTGGAGG | |
| | CAAGGAGATGGCAAAGGGATCACCAGGAACTTCTCCACCTGCTCTCCCAGCA | |
| | CCAAGACCTGCCCCGACGGCCACTGCGATGTTGTGGAGACCCAAGACATCAA | |
| | CATTTGCCCTCAGGACTGCCTCCGGGGCAGCATTGTTGGGGGACACGAGCCT | |
| | GGGGAGCCCCGGGGGATTAAAGCTGGCTATGGCACCTGCAACTGCTTCCCTG | |
| | AGGAGGAGAAGTGCTTCTGCGAGCCCGAAGACATCCAGGATCCACTGTGCGA | |
| | CGAGCTGTGCCGCACGGTGATCGCAGCCGCTGTCCTCTTCTCCTTCATCGTC | |
| | TCGGTGCTGCTGTCTGCCTTCTGCATCCACTGCTACCACAAGTTTGCCCACA | |
| | AGCCACCCATCTCCTCAGCTGAGATGACCTTCCGGAGGCCCGCCCAGGCCTT | |
| | CCCGGTCAGCTACTCCTCTTCCGGTGCCCGCCGGCCCTCGCTGGACTCCATG | |
| | GAGAACCAGGTCTCCGTGGATGCCTTCAAGATCCTGGAGGATCCAAAGTGGG | |
| | AATTCCCTCGGAAGAACTTGGTTCTTGGAAAAACTCTAGGAGAAGGCGAATT | |
| | TGGAAAAGTGGTCAAGGCAACGGCCTTCCATCTGAAAGGCAGAGCAGGGTAC | |
| | ACCACGGTGGCCGTGAAGATGCTGAAAGAGAACGCCTCCCCGAGTGAGCTGC | |
| | GAGACCTGCTGTCAGAGTTCAACGTCCTGAAGCAGGTCAACCACCCACATGT | |
| | CATCAAATTGTATGGGGCCTGCAGCCAGGATGGCCCGCTCCTCCTCATCGTG | |
| | GAGTACGCCAAATACGGCTCCCTGCGGGGCTTCCTCCGCGAGAGCCGCAAAG | |
| | TGGGGCCTGGCTACCTGGGCAGTGGAGGCAGCCGCAACTCCAGCTCCCTGGA | |
| | CCACCCGGATGAGCGGGCCCTCACCATGGGCGACCTCATCTCATTTGCCTGG | |
| | CAGATCTCACAGGGGATGCAGTATCTGGCCGAGATGAAGCTCGTTCATCGGG | |
| | ACTTGGCAGCCAGAAACATCCTGGTAGCTGAGGGGCGGAAGATGAAGATTTC | |
| | GGATTTCGGCTTGTCCCGAGATGTTTATGAAGAGGATTCCTACGTGAAGAGG | |
| | AGCCAGGGTCGGATTCCAGTTAAATGGATGGCAATTGAATCCCTTTTTGATC | |
| | ATATCTACACCACGCAAAGTGATGTATGGTCTTTTGGTGTCCTGCTGTGGGA | |
| | GATCGTGACCCTAGGGGGAAACCCCTATCCTGGGATTCCTCCTGAGCGGCTC | |
| | TTCAACCTTCTGAAGACCGGCCACCGGATGGAGAGGCCAGACAACTGCAGCG | |
| | AGGAGATGTACCGCCTGATGCTGCAATGCTGGAAGCAGGAGCCGGACAAAAG | |
| | GCCGGTGTTTGCGGACATCAGCAAAGACCTGGAGAAGATGATGGTTAAGAGG | |
| | AGAGACTACTTGGACCTTGCGGCGTCCACTCCATCTGACTCCCTGATTTATG | |
| | ACGACGGCCTCTCAGAGGAGGAGACACCGCTGGTGGACTGTAATAATGCCCC | |
| | CCTCCCTCGAGCCCTCCCTTCCACATGGATTGAAAACAAACTCTATGGTAGA | |
| | ATTTCCCATGCATTTACTAGATTCTAG | |
| SORBS1- | ATGAGTTCTGAATGTGATGGTGGTTCCAAAGCTGTGATGAATGGCTTGGCAC | 18 |
| RET | CTGGCAGCAATGGGCAAGACAAAGACATGGATCTTACAAAAATCTGCACTGG | |
| | GAAGGGAGCGGTGACTCTCCGGGCCTCGTCTTCCTACAGGGAAACCCCAAGC | |
| | AGTAGCCCTGCGAGCCCTCAGGAAACCCGGCAACACGAAAGCAAACCAGATG | |
| | AGTGGAGGCTTTCTTCCAGTGCTGATGCCAATGGAAATGCCCAGCCCTCTTC | |
| | ACTCGCTGCCAAGGGCTACAGAAGTGTGCATCCCAACCTTCCTTCTGACAAG | |
| | TCCCAGGATTCCAGTCCTCTACTAAATGAAGTTTCTTCTTCCCTTATTGGAA | |
| | CTGATTCCCAAGCCTTTCCATCAGTTAGCAAGCCTTCATCCGCCTATCCCTC | |
| | CACAACGATTGTCAATCCTACTATTGTGCTCTTGCAACACAATCGAGAACAG | |
| | CAAAAACGACTCAGTAGCCTTTCAGATCCTGTCTCAGAAAGAAGAGTGGGAG | |
| | AGCAGGACTCAGCACCAACCCAGGAAAAACCCACCTCACCTGGCAAGGCTAT | |
| | TGAAAAAAGAGCAAAGGATGACAGTAGGCGGGTGGTGAAGAGCACTCAGGAC | |
| | TTAAGCGATGTTTCCATGGATGAAGTGGGCATCCCACTCCGGAACACTGAGA | |
| | GATCAAAAGACTGGTACAAGACTATGTTTAAACAGATCCACAAACTGAACAG | |
| | AGATGATGATTCAGATCTGTACTCTCCCAGATACTCATTTTCTGAAGACACA | |
| | AAATCTCCCCTTTCTGTGCCTCGCTCAAAAAGTGAGATGAGCTACATTGATG | |
| | GTGAGAAGGTAGTCAAGAGGTCGGCCACACTACCCCTCCCAGCCCGCTCTTC | |
| | CTCACTGAAGTCAAGCTCAGAAAGAAATGACTGGGAACCCCCAGATAAGAAA | |
| | GTAGACACAAGAAAATATCGTGCAGAGCCCAAGAGCATTTACGAATATCAGC | |
| | CTGGCAAGTCTTCCGTTCTGACCAACGAAAAGATGAGCTCAGCCATCAGCCC | |
| | TACTCCGGAAATTTCTTCAGAGACTCCTGGATATATATATTCTTCCAACTTC | |
| | CATGCAGTGAAGAGGGAATCAGACGGGGCTCCTGGGGATCTCACTAGCTTGG | |
| | AGAATGAGAGACAAATTTATAAAAGTGTCTTGGAAGGTGGTGACATCCCTCT | |
| | TCAGGGCCTGAGTGGGCTCAAGCGACCATCCAGCTCTGCTTCCACTAAAGAT | |
| | TCAGAATCGCCAAGACATTTTATACCAGCTGATTACTTGGAATCCACGGAAG | |
| | AATTTATTCGAAGACGTCATGATGATAAAGAGATGAGACCTGCCAGAGCCAA | |
| | ATTTGACTTTAAAGCTCAGACACTAAAGGAGCTTCCTCTGCAGAAGGGAGAT | |
| | ATTGTTTACATTTATAAGCAAATTGATCAGAACTGGTATGAAGGAGAACACC | |
| | ACGGCCGGGTGGGAATCTTCCCACGCACCTACATCGAGCTTCTTCCTCCTGC | |
| | TGAGAAGGCACAGCCCAAAAAGTTGACACCAGTGCAGGTTTTGGAATATGGA | |
| | GAAGCTATTGCTAAGTTTAACTTTAATGGTGATACACAAGTAGAAATGTCCT | |
| | TCAGAAAGGGTGAGAGGATCACACTGCTCCGGCAGGTAGATGAGAACTGGTA | |
| | CGAAGGGAGGATCCCGGGGACATCCCGACAAGGCATCTTCCCCATCACCTAC | |
| | GTGGATGTGATCAAGCGACCACTGGTGAAAAACCCTGTGGATTACATGGACC | |
| | TGCCTTTCTCCTCCTCCCCAAGTCGCAGTGCCACTGCAAGCCCACAGCAACC | |
| | TCAAGCCCAGCAGCGAAGAGTCACCCCCGACAGGAGTCAAACCTCACAAGAT | |
| | TTATTTAGCTATCAAGCATTATATAGCTATATACCACAGAATGATGATGAGT | |
| | TGGAACTCCGCGATGGAGATATCGTTGATGTCATGGAAAAATGTGACGATGG | |
| | ATGGTTTGTTGATCCACTGTGCGACGAGCTGTGCCGCACGGTGATCGCAGCC | |
| | GCTGTCCTCTTCTCCTTCATCGTCTCGGTGCTGCTGTCTGCCTTCTGCATCC | |
| | ACTGCTACCACAAGTTTGCCCACAAGCCACCCATCTCCTCAGCTGAGATGAC | |
| | CTTCCGGAGGCCCGCCCAGGCCTTCCCGGTCAGCTACTCCTCTTCCGGTGCC | |
| | CGCCGGCCCTCGCTGGACTCCATGGAGAACCAGGTCTCCGTGGATGCCTTCA | |
| | AGATCCTGGAGGATCCAAAGTGGGAATTCCCTCGGAAGAACTTGGTTCTTGG | |
| | AAAAACTCTAGGAGAAGGCGAATTTGGAAAAGTGGTCAAGGCAACGGCCTTC | |
| | CATCTGAAAGGCAGAGCAGGGTACACCACGGTGGCCGTGAAGATGCTGAAAG | |
| | AGAACGCCTCCCCGAGTGAGCTGCGAGACCTGCTGTCAGAGTTCAACGTCCT | |
| | GAAGCAGGTCAACCACCCACATGTCATCAAATTGTATGGGGCCTGCAGCCAG | |
| | GATGGCCCGCTCCTCCTCATCGTGGAGTACGCCAAATACGGCTCCCTGCGGG | |
| | GCTTCCTCCGCGAGAGCCGCAAAGTGGGGCCTGGCTACCTGGGCAGTGGAGG | |
| | CAGCCGCAACTCCAGCTCCCTGGACCACCCGGATGAGCGGGCCCTCACCATG | |
| | GGCGACCTCATCTCATTTGCCTGGCAGATCTCACAGGGGATGCAGTATCTGG | |
| | CCGAGATGAAGCTCGTTCATCGGGACTTGGCAGCCAGAAACATCCTGGTAGC | |
| | TGAGGGGCGGAAGATGAAGATTTCGGATTTCGGCTTGTCCCGAGATGTTTAT | |
| | GAAGAGGATTCCTACGTGAAGAGGAGCCAGGGTCGGATTCCAGTTAAATGGA | |
| | TGGCAATTGAATCCCTTTTTGATCATATCTACACCACGCAAAGTGATGTATG | |
| | GTCTTTTGGTGTCCTGCTGTGGGAGATCGTGACCCTAGGGGGAAACCCCTAT | |
| | CCTGGGATTCCTCCTGAGCGGCTCTTCAACCTTCTGAAGACCGGCCACCGGA | |
| | TGGAGAGGCCAGACAACTGCAGCGAGGAGATGTACCGCCTGATGCTGCAATG | |
| | CTGGAAGCAGGAGCCGGACAAAAGGCCGGTGTTTGCGGACATCAGCAAAGAC | |
| | CTGGAGAAGATGATGGTTAAGAGGAGAGACTACTTGGACCTTGCGGCGTCCA | |
| | CTCCATCTGACTCCCTGATTTATGACGACGGCCTCTCAGAGGAGGAGACACC | |
| | GCTGGTGGACTGTAATAATGCCCCCCTCCCTCGAGCCCTCCCTTCCACATGG | |
| | ATTGAAAACAAACTCTATGGTAGAATTTCCCATGCATTTACTAGATTCTAG | |
| MPRIP- | ATGTCGGCAGCCAAGGAGAACCCGTGCAGGAAATTCCAGGCCAACATCTTCA | 19 |
| RET | ACAAGAGCAAGTGTCAGAACTGCTTCAAGCCCCGCGAGTCGCATCTGCTCAA | |
| | CGACGAGGACCTGACGCAGGCAAAACCCATTTATGGCGGTTGGCTGCTCCTG | |
| | GCTCCAGATGGGACCGACTTTGACAACCCAGTGCACCGGTCTCGGAAATGGC | |
| | AGCGACGGTTCTTCATCCTTTACGAGCACGGCCTCTTGCGCTACGCCCTGGA | |
| | TGAGATGCCCACGACCCTTCCTCAGGGCACCATCAACATGAACCAGTGCACA | |
| | GATGTGGTGGATGGGGAGGGCCGCACGGGCCAGAAGTTCTCCCTGTGTATTC | |
| | TGACGCCTGAGAAGGAGCATTTCATCCGGGCGGAGACCAAGGAGATCGTCAG | |
| | TGGGTGGCTGGAGATGCTCATGGTCTATCCCCGGACCAACAAGCAGAATCAG | |
| | AAGAAGAAACGGAAAGTGGAGCCCCCCACACCACAGGAGCCTGGGCCTGCCA | |
| | AGGTGGCTGTTACCAGCAGCAGCAGCAGCAGCAGCAGCAGCAGCAGCATCCC | |
| | CAGTGCTGAGAAAGTCCCCACCACCAAGTCCACACTCTGGCAGGAAGAAATG | |
| | AGGACCAAGGACCAGCCAGATGGCAGCAGCCTGAGTCCAGCTCAGAGTCCCA | |
| | GCCAGAGCCAGCCTCCTGCTGCCAGCTCCCTGCGGGAACCTGGGCTAGAGAG | |
| | CAAAGAAGAGGAGAGCGCCATGAGTAGCGACCGCATGGACTGTGGCCGCAAA | |
| | GTCCGGGTGGAGAGCGGCTACTTCTCTCTGGAGAAGACCAAACAGGACTTGA | |
| | AGGCTGAAGAACAGCAGCTGCCCCCGCCGCTCTCCCCTCCCAGCCCCAGCAC | |
| | CCCCAACCACAGGAGGTCCCAGGTGATTGAAAAGTTTGAGGCCTTGGACATT | |
| | GAGAAGGCAGAGCACATGGAGACCAATGCAGTGGGGCCCTCACCATCCAGCG | |
| | ACACACGCCAGGGCCGCAGCGAGAAGAGGGCGTTCCCTAGGAAGCGGGACTT | |
| | CACCAATGAAGCCCCCCCAGCTCCTCTCCCAGACGCCTCGGCTTCCCCCCTG | |
| | TCTCCACACCGAAGAGCCAAGTCACTGGACAGGAGGTCCACGGAGCCCTCCG | |
| | TGACGCCCGACCTGCTGAATTTCAAGAAAGGCTGGCTGACTAAGCAGTATGA | |
| | GGACGGCCAGTGGAAGAAACACTGGTTTGTCCTCGCCGATCAAAGCCTGAGA | |
| | TACTACAGGGATTCAGTGGCTGAGGAGGCAGCCGACTTGGATGGAGAAATTG | |
| | ACTTGTCCGCATGTTACGATGTCACAGAGTATCCAGTTCAGAGAAACTATGG | |
| | CTTCCAGATACATACAAAGGAGGGCGAGTTTACCCTGTCGGCCATGACATCT | |
| | GGGATTCGGCGGAACTGGATCCAGACCATCATGAAGCACGTGCACCCGACCA | |
| | CTGCCCCGGATGTGACCAGCTCGTTGCCAGAGGAAAAAAACAAGAGCAGCTG | |
| | CTCTTTTGAGACCTGCCCGAGGCCTACTGAGAAGCAAGAGGCAGAGCTGGGG | |
| | GAGCCGGACCCTGAGCAGAAGAGGAGCCGCGCACGGGAGCGGAGGCGAGAGG | |
| | GCCGCTCCAAGACCTTTGACTGGGCTGAGTTCCGTCCCATCCAGCAGGCCCT | |
| | GGCTCAGGAGCGGGTGGGCGGCGTGGGGCCTGCTGACACCCACGAGCCCCTG | |
| | CGCCCTGAGGCGGAGCCTGGGGAGCTGGAGCGGGAGCGTGCACGGAGGCGGG | |
| | AGGAGCGCCGCAAGCGCTTCGGGATGCTCGACGCCACAGACGGGCCAGGCAC | |
| | TGAGGATGCAGCCCTGCGCATGGAGGTGGACCGGAGCCCAGGGCTGCCTATG | |
| | AGCGACCTCAAAACGCATAACGTCCACGTGGAGATTGAGCAGCGGTGGCATC | |
| | AGGTGGAGACCACACCTCTCCGGGAAGAGAAGCAGGTGCCCATCGCCCCCGT | |
| | CCACCTGTCTTCTGAAGATGGGGGTGACCGGCTCTCCACACACGAGCTGACC | |
| | TCTCTGCTCGAGAAGGAGCTGGAGCAGAGCCAGAAGGAGGCCTCAGACCTTC | |
| | TGGAGCAGAACCGGCTCCTGCAGGACCAGCTGAGGGTGGCCCTGGGCCGGGA | |
| | GCAGAGCGCCCGTGAGGGCTACGTGCTGCAGGCCACGTGCGAGCGAGGGTTT | |
| | GCAGCAATGGAAGAAACGCACCAGAAGAAGATTGAAGATCTCCAGAGGCAGC | |
| | ACCAGCGGGAGCTAGAGAAACTTCGAGAAGAGAAAGACCGCCTCCTAGCCGA | |
| | GGAGACAGCGGCCACCATCTCAGCCATCGAAGCCATGAAGAACGCCCACCGG | |
| | GAGGAAATGGAGCGGGAGCTGGAGAAGAGCCAGCGGTCCCAGATCAGCAGCG | |
| | TCAACTCGGATGTTGAGGCCCTGCGGCGCCAGTACCTGGAGGAGCTGCAGTC | |
| | GGTGCAGCGGGAACTGGAGGTCCTCTCGGAGCAGTACTCGCAGAAGTGCCTG | |
| | GAGAATGCCCATCTGGCCCAGGCGCTGGAGGCCGAGCGGCAGGCCCTGCGGC | |
| | AGTGCCAGCGTGAGAACCAGGAGCTCAATGCCCACAACCAGGAGCTGAACAA | |
| | CCGCCTGGCTGCAGAGATCACACGGTTGCGGACGCTGCTGACTGGGGACGGC | |
| | GGTGGGGAGGCCACTGGGTCACCCCTTGCACAGGGCAAGGATGCCTATGAAC | |
| | TAGAGGTCTTATTGCGGGTAAAGGAATCGGAAATACAGTACCTGAAACAGGA | |
| | GATTAGCTCCCTCAAGGATGAGCTGCAGACGGCACTGCGGGACAAGAAGTAC | |
| | GCAAGTGACAAGTACAAAGACATCTACACAGAGCTCAGCATCGCGAAGGCTA | |
| | AGGCTGACTGTGACATCAGCAGGTTGAAGGAGCAGCTCAAGGCTGCAACGGA | |
| | AGCACTGGGGGAGAAGTCCCCTGACAGTGCCACGGTGTCCGGATATGATATA | |
| | ATGAAATCTAAAAGCAACCCTGACTTCTTGAAGAAAGACAGATCCTGTGTCA | |
| | CCCGGCAACTCAGAAACATCAGGTCCAAGGAGGATCCAAAGTGGGAATTCCC | |
| | TCGGAAGAACTTGGTTCTTGGAAAAACTCTAGGAGAAGGCGAATTTGGAAAA | |
| | GTGGTCAAGGCAACGGCCTTCCATCTGAAAGGCAGAGCAGGGTACACCACGG | |
| | TGGCCGTGAAGATGCTGAAAGAGAACGCCTCCCCGAGTGAGCTGCGAGACCT | |
| | GCTGTCAGAGTTCAACGTCCTGAAGCAGGTCAACCACCCACATGTCATCAAA | |
| | TTGTATGGGGCCTGCAGCCAGGATGGCCCGCTCCTCCTCATCGTGGAGTACG | |
| | CCAAATACGGCTCCCTGCGGGGCTTCCTCCGCGAGAGCCGCAAAGTGGGGCC | |
| | TGGCTACCTGGGCAGTGGAGGCAGCCGCAACTCCAGCTCCCTGGACCACCCG | |
| | GATGAGCGGGCCCTCACCATGGGCGACCTCATCTCATTTGCCTGGCAGATCT | |
| | CACAGGGGATGCAGTATCTGGCCGAGATGAAGCTCGTTCATCGGGACTTGGC | |
| | AGCCAGAAACATCCTGGTAGCTGAGGGGCGGAAGATGAAGATTTCGGATTTC | |
| | GGCTTGTCCCGAGATGTTTATGAAGAGGATTCCTACGTGAAGAGGAGCCAGG | |
| | GTCGGATTCCAGTTAAATGGATGGCAATTGAATCCCTTTTTGATCATATCTA | |
| | CACCACGCAAAGTGATGTATGGTCTTTTGGTGTCCTGCTGTGGGAGATCGTG | |
| | ACCCTAGGGGGAAACCCCTATCCTGGGATTCCTCCTGAGCGGCTCTTCAACC | |
| | TTCTGAAGACCGGCCACCGGATGGAGAGGCCAGACAACTGCAGCGAGGAGAT | |
| | GTACCGCCTGATGCTGCAATGCTGGAAGCAGGAGCCGGACAAAAGGCCGGTG | |
| | TTTGCGGACATCAGCAAAGACCTGGAGAAGATGATGGTTAAGAGGAGAGACT | |
| | ACTTGGACCTTGCGGCGTCCACTCCATCTGACTCCCTGATTTATGACGACGG | |
| | CCTCTCAGAGGAGGAGACACCGCTGGTGGACTGTAATAATGCCCCCCTCCCT | |
| | CGAGCCCTCCCTTCCACATGGATTGAAAACAAACTCTATGGTAGAATTTCCC | |
| | ATGCATTTACTAGATTCTAG | |
| TFG-RET | ATGAACGGACAGTTGGATCTAAGTGGGAAGCTAATCATCAAAGCTCAACTTG | 20 |
| | GGGAGGATATTCGGCGAATTCCTATTCATAATGAAGATATTACTTATGATGA | |
| | ATTAGTGCTAATGATGCAACGAGTTTTCAGAGGAAAACTTCTGAGTAATGAT | |
| | GAAGTAACAATAAAGTATAAAGATGAAGATGGAGATCTTATAACAATTTTTG | |
| | ATAGTTCTGACCTTTCCTTTGCAATTCAGTGCAGTAGGATACTGAAACTGAC | |
| | ATTATTTGTTAATGGCCAGCCAAGACCCCTTGAATCAAGTCAGGTGAAATAT | |
| | CTCCGTCGAGAACTGATAGAACTTCGAAATAAAGTGAATCGTTTATTGGATA | |
| | GCTTGGAACCACCTGGAGAACCAGGACCTTCCACCAATATTCCTGAAAATGA | |
| | TACTGTGGATGGTAGGGAAGAAAAGTCTGCTTCTGATTCTTCTGGAAAACAG | |
| | TCTACTCAGGTTATGGCAGCAAGTATGTCTGCTTTTGATCCTTTAAAAAACC | |
| | AAGATGAAATCAATAAAAATGTTATGTCAGCGTTTGGCTTAACAGATGATCA | |
| | GGTTTCAGATCCACTGTGCGACGAGCTGTGCCGCACGGTGATCGCAGCCGCT | |
| | GTCCTCTTCTCCTTCATCGTCTCGGTGCTGCTGTCTGCCTTCTGCATCCACT | |
| | GCTACCACAAGTTTGCCCACAAGCCACCCATCTCCTCAGCTGAGATGACCTT | |
| | CCGGAGGCCCGCCCAGGCCTTCCCGGTCAGCTACTCCTCTTCCGGTGCCCGC | |
| | CGGCCCTCGCTGGACTCCATGGAGAACCAGGTCTCCGTGGATGCCTTCAAGA | |
| | TCCTGGAGGATCCAAAGTGGGAATTCCCTCGGAAGAACTTGGTTCTTGGAAA | |
| | AACTCTAGGAGAAGGCGAATTTGGAAAAGTGGTCAAGGCAACGGCCTTCCAT | |
| | CTGAAAGGCAGAGCAGGGTACACCACGGTGGCCGTGAAGATGCTGAAAGAGA | |
| | ACGCCTCCCCGAGTGAGCTGCGAGACCTGCTGTCAGAGTTCAACGTCCTGAA | |
| | GCAGGTCAACCACCCACATGTCATCAAATTGTATGGGGCCTGCAGCCAGGAT | |
| | GGCCCGCTCCTCCTCATCGTGGAGTACGCCAAATACGGCTCCCTGCGGGGCT | |
| | TCCTCCGCGAGAGCCGCAAAGTGGGGCCTGGCTACCTGGGCAGTGGAGGCAG | |
| | CCGCAACTCCAGCTCCCTGGACCACCCGGATGAGCGGGCCCTCACCATGGGC | |
| | GACCTCATCTCATTTGCCTGGCAGATCTCACAGGGGATGCAGTATCTGGCCG | |
| | AGATGAAGCTCGTTCATCGGGACTTGGCAGCCAGAAACATCCTGGTAGCTGA | |
| | GGGGCGGAAGATGAAGATTTCGGATTTCGGCTTGTCCCGAGATGTTTATGAA | |
| | GAGGATTCCTACGTGAAGAGGAGCCAGGGTCGGATTCCAGTTAAATGGATGG | |
| | CAATTGAATCCCTTTTTGATCATATCTACACCACGCAAAGTGATGTATGGTC | |
| | TTTTGGTGTCCTGCTGTGGGAGATCGTGACCCTAGGGGGAAACCCCTATCCT | |
| | GGGATTCCTCCTGAGCGGCTCTTCAACCTTCTGAAGACCGGCCACCGGATGG | |
| | AGAGGCCAGACAACTGCAGCGAGGAGATGTACCGCCTGATGCTGCAATGCTG | |
| | GAAGCAGGAGCCGGACAAAAGGCCGGTGTTTGCGGACATCAGCAAAGACCTG | |
| | GAGAAGATGATGGTTAAGAGGAGAGACTACTTGGACCTTGCGGCGTCCACTC | |
| | CATCTGACTCCCTGATTTATGACGACGGCCTCTCAGAGGAGGAGACACCGCT | |
| | GGTGGACTGTAATAATGCCCCCCTCCCTCGAGCCCTCCCTTCCACATGGATT | |
| | GAAAACAAACTCTATGGTAGAATTTCCCATGCATTTACTAGATTCTAG | |
| SPECC1L- | ATGAAGAAAGCAAGCAGGAGTGTTGGCTCAGTGCCTAAAGTGTCTGCAATAA | 21 |
| RET | GTAAAACGCAAACAGCAGAAAAAATTAAACCTGAAAACAGCTCTTCAGCATC | |
| | TACGGGAGGCAAACTTGTAAAACCTGGAACAGCAGCATCATTGTCAAAGACC | |
| | AAGAGCAGTGATGACCTTTTAGCTGGAATGGCCGGAGGGGTAACGGTGACTA | |
| | ATGGTGTTAAAGGAAAGAAAAGCACCTGCCCATCTGCAGCACCTTCAGCATC | |
| | TGCCCCTGCCATGACCACCGTGGAGAACAAATCCAAGATTAGCACAGGCACA | |
| | GCTTCTTCAACCAAGCGGAGCACTTCTACAGGTAATAAAGAATCCAGTTCTA | |
| | CTAGAGAAAGATTACGTGAACGTACCCGATTAAACCAGAGCAAAAAACTACC | |
| | TTCTGCAGGTCAGGGAGCTAATGACATGGCATTGGCCAAACGTTCCCGCAGT | |
| | CGAACTGCTACAGAATGTGACGTTCGTATGAGCAAGTCTAAGTCAGACAATC | |
| | AGATCAGTGACAGAGCTGCTTTGGAGGCCAAAGTGAAGGATCTTCTCACGCT | |
| | GGCAAAAACCAAAGACGTAGAAATTTTACATTTGAGAAATGAACTGCGAGAC | |
| | ATGCGTGCCCAGCTGGGCATTAATGAGGATCATTCTGAGGGTGATGAAAAAT | |
| | CTGAGAAGGAAACTATTATGGCTCACCAGCCGACTGATGTGGAGTCCACTTT | |
| | ATTGCAGTTGCAGGAACAGAATACTGCCATCCGTGAAGAACTCAACCAGCTG | |
| | AAAAATGAAAACAGAATGTTAAAGGACAGGTTGAATGCATTGGGCTTTTCCC | |
| | TAGAGCAGAGGTTAGACAATTCTGAAAAACTGTTTGGCTATCAGTCCCTGAG | |
| | CCCAGAAATCACCCCTGGTAACCAGAGCGATGGAGGAGGAACTCTGACTTCT | |
| | TCAGTGGAAGGCTCTGCCCCTGGCTCAGTGGAGGATCTCTTGAGTCAGGATG | |
| | AAAATACACTAATGGACCATCAGCACAGTAACTCCATGGACAATTTAGACAG | |
| | TGAGTGCAGTGAGGTCTACCAGCCCCTCACATCGAGCGATGATGCGCTGGAT | |
| | GCACCATCCTCCTCAGAGTCGGAAGGCATCCCCAGCATAGAGCGCTCCCGGA | |
| | AGGGGAGCAGCGGGAATGCCAGTGAAGTGTCCGTGGCTTGCCTGACTGAACG | |
| | GATACACCAGATGGAAGAGAACCAACACAGTACAAGTGAGGAACTCCAGGCA | |
| | ACCCTGCAAGAGCTAGCTGATTTACAGCAGATTACCCAGGAACTGAATAGTG | |
| | AAAACGAAAGGCTTGGAGAAGAGAAGGTTATTCTGATGGAGTCTTTATGTCA | |
| | GCAGAGCGATAAGTTGGAACACTTTAGTCGACAGATTGAATACTTCCGCTCT | |
| | CTTCTAGATGAGCATCACATTTCTTATGTCATAGATGAAGATGTAAAAAGTG | |
| | GGCGCTATATGGAATTAGAGCAACGTTACATGGACCTCGCTGAGAATGCCCG | |
| | TTTTGAACGGGAGCAGCTTCTTGGTGTCCAGCAGCATTTAAGCAATACTTTG | |
| | AAAATGGCAGAACAAGACAATAAGGAAGCTCAAGAAATGATAGGGGCACTCA | |
| | AAGAACGCAGTCACCATATGGAGCGAATTATTGAGTCTGAGCAGAAAGGAAA | |
| | AGCAGCCTTGGCAGCCACGTTAGAGGAATACAAAGCCACAGTGGCCAGTGAC | |
| | CAGATAGAGATGAATCGCCTGAAGGCTCAGCTGGAGAATGAAAAGCAGAAAG | |
| | TGGCAGAGCTGTATTCTATCCATAACTCTGGAGACAAATCTGATATTCAGGA | |
| | CCTCCTGGAGAGTGTCAGGCTGGACAAAGAAAAAGCAGAGACTTTGGCTAGT | |
| | AGCTTGCAGGAAGATCTGGCTCATACCCGAAATGATGCCAATCGATTACAGG | |
| | ATGCCATTGCTAAGGTAGAGGATGAATACCGAGCCTTCCAAGAAGAAGCTAA | |
| | GAAACAAATTGAAGATTTGAATATGACGTTAGAAAAATTAAGATCAGACCTG | |
| | GATGAAAAAGAAACAGAAAGGAGTGACATGAAAGAAACCATCTTTGAACTTG | |
| | AAGATGAAGTAGAACAACATCGTGCTGTGAAACTTCATGACAACCTCATTAT | |
| | TTCTGATCTAGAGAATACAGTTAAAAAACTCCAGGACCAAAAGCACGACATG | |
| | GAAAGAGAAATAAAGACACTCCACAGAAGACTTCGGGAAGAATCTGCGGAAT | |
| | GGCGGCAGTTTCAGGCTGATCTCCAGACTGCAGTAGTCATTGCAAATGACAT | |
| | TAAATCTGAAGCCCAAGAGGAGATTGGTGATCTAAAGCGCCGGTTACATGAG | |
| | GCTCAAGAAAAAAATGAGAAACTCACAAAAGAATTGGAGGAAATAAAGTCAC | |
| | GCAAGCAAGAGGAGGAGCGAGGCCGGGTATACAATTACATGAATGCCGTTGA | |
| | GAGAGATTTGGCAGCCTTAAGGCAGGGAATGGGACTGAGTAGAAGGTCCTCG | |
| | ACTTCCTCAGAGCCAACTCCTACAGTAAAAACCCTCATCAAGTCCTTTGACA | |
| | GTGCATCTCAAGTACCAAACCCTGCTGCAGCTGCAATTCCTCGAACGCCCCT | |
| | GAGCCCAAGTCCTATGAAAACCCCTCCTGCAGCAGCTGTGTCCCCTATGCAG | |
| | GAGGATCCAAAGTGGGAATTCCCTCGGAAGAACTTGGTTCTTGGAAAAACTC | |
| | TAGGAGAAGGCGAATTTGGAAAAGTGGTCAAGGCAACGGCCTTCCATCTGAA | |
| | AGGCAGAGCAGGGTACACCACGGTGGCCGTGAAGATGCTGAAAGAGAACGCC | |
| | TCCCCGAGTGAGCTGCGAGACCTGCTGTCAGAGTTCAACGTCCTGAAGCAGG | |
| | TCAACCACCCACATGTCATCAAATTGTATGGGGCCTGCAGCCAGGATGGCCC | |
| | GCTCCTCCTCATCGTGGAGTACGCCAAATACGGCTCCCTGCGGGGCTTCCTC | |
| | CGCGAGAGCCGCAAAGTGGGGCCTGGCTACCTGGGCAGTGGAGGCAGCCGCA | |
| | ACTCCAGCTCCCTGGACCACCCGGATGAGCGGGCCCTCACCATGGGCGACCT | |
| | CATCTCATTTGCCTGGCAGATCTCACAGGGGATGCAGTATCTGGCCGAGATG | |
| | AAGCTCGTTCATCGGGACTTGGCAGCCAGAAACATCCTGGTAGCTGAGGGGC | |
| | GGAAGATGAAGATTTCGGATTTCGGCTTGTCCCGAGATGTTTATGAAGAGGA | |
| | TTCCTACGTGAAGAGGAGCCAGGGTCGGATTCCAGTTAAATGGATGGCAATT | |
| | GAATCCCTTTTTGATCATATCTACACCACGCAAAGTGATGTATGGTCTTTTG | |
| | GTGTCCTGCTGTGGGAGATCGTGACCCTAGGGGGAAACCCCTATCCTGGGAT | |
| | TCCTCCTGAGCGGCTCTTCAACCTTCTGAAGACCGGCCACCGGATGGAGAGG | |
| | CCAGACAACTGCAGCGAGGAGATGTACCGCCTGATGCTGCAATGCTGGAAGC | |
| | AGGAGCCGGACAAAAGGCCGGTGTTTGCGGACATCAGCAAAGACCTGGAGAA | |
| | GATGATGGTTAAGAGGAGAGACTACTTGGACCTTGCGGCGTCCACTCCATCT | |
| | GACTCCCTGATTTATGACGACGGCCTCTCAGAGGAGGAGACACCGCTGGTGG | |
| | ACTGTAATAATGCCCCCCTCCCTCGAGCCCTCCCTTCCACATGGATTGAAAA | |
| | CAAACTCTATGGTAGAATTTCCCATGCATTTACTAGATTCTAG | |
| REEP3- | ATGGTGTCCTGGATGATCTCCAGAGCCGTGGTGCTGGTGTTTGGAATGCTTT | 22 |
| RET | ATCCTGCATATTATTCATACAAAGCTGTGAAAACAAAAAACGTGAAGGAATA | |
| | TGTTCGATGGATGATGTACTGGATTGTTTTTGCTCTCTATACTGTGATTGAA | |
| | ACAGTAGCCGATCAAACAGTTGCTTGGTTTCCCCTGTACTATGAGCTGAAGA | |
| | TTGCTTTTGTCATATGGCTGCTTTCTCCCTATACCAAAGGAGCAAGTTTAAT | |
| | ATATAGAAAATTCCTTCATCCACTTCTTTCTTCAAAGGAAAGGGAGATTGAT | |
| | GATTATATTGTACAAGCAAAGGAACGAGGCTATGAAACCATGGTAAACTTTG | |
| | GACGGCAAGGTTTAAACCTTGCAGCTACTGCTGCTGTTACTGCAGCAGTAAA | |
| | GGAGGATCCAAAGTGGGAATTCCCTCGGAAGAACTTGGTTCTTGGAAAAACT | |
| | CTAGGAGAAGGCGAATTTGGAAAAGTGGTCAAGGCAACGGCCTTCCATCTGA | |
| | AAGGCAGAGCAGGGTACACCACGGTGGCCGTGAAGATGCTGAAAGAGAACGC | |
| | CTCCCCGAGTGAGCTGCGAGACCTGCTGTCAGAGTTCAACGTCCTGAAGCAG | |
| | GTCAACCACCCACATGTCATCAAATTGTATGGGGCCTGCAGCCAGGATGGCC | |
| | CGCTCCTCCTCATCGTGGAGTACGCCAAATACGGCTCCCTGCGGGGCTTCCT | |
| | CCGCGAGAGCCGCAAAGTGGGGCCTGGCTACCTGGGCAGTGGAGGCAGCCGC | |
| | AACTCCAGCTCCCTGGACCACCCGGATGAGCGGGCCCTCACCATGGGCGACC | |
| | TCATCTCATTTGCCTGGCAGATCTCACAGGGGATGCAGTATCTGGCCGAGAT | |
| | GAAGCTCGTTCATCGGGACTTGGCAGCCAGAAACATCCTGGTAGCTGAGGGG | |
| | CGGAAGATGAAGATTTCGGATTTCGGCTTGTCCCGAGATGTTTATGAAGAGG | |
| | ATTCCTACGTGAAGAGGAGCCAGGGTCGGATTCCAGTTAAATGGATGGCAAT | |
| | TGAATCCCTTTTTGATCATATCTACACCACGCAAAGTGATGTATGGTCTTTT | |
| | GGTGTCCTGCTGTGGGAGATCGTGACCCTAGGGGGAAACCCCTATCCTGGGA | |
| | TTCCTCCTGAGCGGCTCTTCAACCTTCTGAAGACCGGCCACCGGATGGAGAG | |
| | GCCAGACAACTGCAGCGAGGAGATGTACCGCCTGATGCTGCAATGCTGGAAG | |
| | CAGGAGCCGGACAAAAGGCCGGTGTTTGCGGACATCAGCAAAGACCTGGAGA | |
| | AGATGATGGTTAAGAGGAGAGACTACTTGGACCTTGCGGCGTCCACTCCATC | |
| | TGACTCCCTGATTTATGACGACGGCCTCTCAGAGGAGGAGACACCGCTGGTG | |
| | GACTGTAATAATGCCCCCCTCCCTCGAGCCCTCCCTTCCACATGGATTGAAA | |
| | ACAAACTCTATGGTAGAATTTCCCATGCATTTACTAGATTCTAG | |
| RRBP1- | ATGGATATTTACGACACTCAAACCTTGGGGGTTGTGGTCTTTGGAGGATTCA | 23 |
| RET | TGGTTGTTTCTGCCATTGGCATCTTCCTGGTGTCGACTTTCTCCATGAAGGA | |
| | AACGTCATATGAAGAAGCCCTAGCCAACCAGCGCAAGGAGATGGCGAAAACT | |
| | CACCACCAGAAAGTCGAGAAGAAAAAGAAGGAGAAAACAGTGGAGAAGAAAG | |
| | GAAAGACCAAGAAAAAGGAAGAGAAACCTAATGGGAAGATACCTGATCATGA | |
| | TCCAGCCCCCAATGTGACTGTCCTCCTTCGAGAACCAGTGCGGGCTCCTGCT | |
| | GTGGCTGTGGCTCCAACCCCAGTGCAGCCCCCCATTATCGTTGCTCCTGTCG | |
| | CCACAGTTCCAGCCATGCCCCAGGAGAAGCTGGCCTCCTCCCCCAAGGACAA | |
| | AAAGAAGAAGGAGAAAAAAGTGGCAAAAGTGGAACCAGCTGTCAGCTCTGTA | |
| | GTGAATTCCATCCAGGTTCTCACTTCGAAGGCTGCCATCTTGGAAACTGCTC | |
| | CCAAGGAGGGCAGAAATACAGATGTGGCCCAGAGCCCAGAGGCACCAAAGCA | |
| | AGAGGCTCCTGCCAAGAAGAAGTCTGGTTCAAAGAAAAAAGGGCCCCCAGAT | |
| | GCCGACGGCCCTCTCTACCTCCCCTACAAGACGCTGGTCTCCACGGTTGGGA | |
| | GCATGGTGTTCAACGAGGGCGAGGCCCAGCGGCTCATCGAGATCCTGTCTGA | |
| | GAAGGCTGGCATCATTCAGGACACCTGGCACAAGGCCACTCAGAAGGGTGAC | |
| | CCTGTGGCGATTCTGAAACGCCAGCTGGAAGAGAAGGAAAAACTGCTGGCCA | |
| | CAGAACAGGAAGATGCGGCTGTCGCCAAGAGCAAACTGAGGGAGCTCAACAA | |
| | GGAGATGGCAGCAGAAAAGGCCAAAGCAGCAGCCGGGGAGGCCAAAGTGAAA | |
| | AAGCAGCTGGTGGCCCGGGAGCAGGAGATCACGGCTGTGCAGGCACGCATGC | |
| | AGGCCAGCTACCGGGAGCACGTGAAGGAGGTGCAGCAGCTGCAGGGCAAGAT | |
| | CCGGACTCTTCAGGAGCAGCTGGAGAATGGCCCCAACACGCAGCTGGCCCGC | |
| | CTGCAGCAGGAGAACTCCATCCTGCGGGATGCCTTGAACCAGGCCACGAGCC | |
| | AGGTGGAGAGCAAGCAGAACGCAGAGCTGGCCAAGCTTCGGCAGGAGCTCAG | |
| | CAAGGTCAGCAAAGAGCTGGTGGAGAAGTCAGAGGCTGTGCGGCAAGATGAG | |
| | CAGCAGCGGAAAGCTCTGGAAGCCAAGGCAGCTGCCTTCGAGAAGCAGGTCC | |
| | TGCAGCTGCAGGCGTCCCACAGGGAGAGTGAGGAGGCCCTGCAGAAGCGCCT | |
| | GGACGAGGTCAGCCGGGAGCTGTGCCACACGCAGAGCAGCCACGCCAGCCTC | |
| | CGGGCGGATGCCGAGAAGGCCCAGGAGCAACAGCAGCAGATGGCCGAGCTGC | |
| | ACAGCAAGTTACAGTCCTCCGAGGCGGAGGTGCGCAGCAAATGCGAGGAGCT | |
| | GAGTGGCCTCCACGGGCAGCTCCAGGAGGCCAGGGCGGAGAACTCCCAGCTC | |
| | ACAGAGAGAATCCGTTCCATTGAGGCCCTGCTGGAGGCGGGCCAGGCGCGGG | |
| | ATGCCCAGGACGTCCAGGCCAGCCAGGCGGAGGCTGACCAGCAGCAGACTCG | |
| | CCTCAAGGAGCTGGAGTCCCAGGTGTCGGGTCTGGAGAAGGAGGCCATCGAG | |
| | CTCAGGGAGGCCGTCGAGCAGCAGAAAGTGAAGAACAATGACCTCCGGGAGA | |
| | AGAACTGGAAGGCCATGGAGGCACTGGCCACGGCCGAGCAGGCCTGCAAGGA | |
| | GAAGCTGCTCTCCCTGACCCAGGCCAAGGAGGAATCGGAGAAGCAGCTCTGT | |
| | CTGATTGAGGCGCAGACCATGGAGGCCCTGCTGGCTCTGCTCCCAGAACTCT | |
| | CTGTCTTGGCACAACAGAATTACACCGAGTGGCTGCAGGATCTCAAAGAGAA | |
| | AGGCCCCACGCTGCTGAAGCACCCGCCAGCTCCCGCGGAGCCCTCCTCGGAC | |
| | CTGGCCTCCAAGTTGAGGGAGGCCGAGGAGACGCAGAGCACACTGCAGGCCG | |
| | AGTGTGACCAGTACCGCAGCATCCTGGCGGAGACGGAGGGCATGCTCAGAGA | |
| | CCTGCAGAAGAGCGTGGAGGAGGAGGAGCAGGTGTGGAGGGCCAAGGTGGGC | |
| | GCCGCAGAGGAGGAGCTCCAGAAGTCCCGGGTCACAGTGAAGCATCTCGAAG | |
| | AGATTGTAGAGAAGCTAAAAGGAGAACTTGAAAGTTCGGACCAGGTGAGGGA | |
| | GCACACGTCGCATTTGGAGGCAGAGCTGGAAAAGCACATGGCGGCCGCCAGC | |
| | GCCGAGTGCCAGAACTACGCCAAGGAGGTGGCAGGGCTGAGGCAACTTCTCC | |
| | TAGAATCTCAATCTCAGCTCGATGCCGCCAAGAGCGAAGCCCAGAAACAGAG | |
| | CGATGAGCTTGCCCTGGTCAGGCAGCAGTTGAGTGAAATGAAGAGCCACGTA | |
| | GAGGATGGTGACATAGCTGGGGCCCCAGCTTCCTCCCCAGAGGCGCCCCCAG | |
| | CCGAGCAGGACCCCGTTCAGCTGAAGACGCAGCTGGAGTGGACAGAAGCCAT | |
| | CCTGGAGGATGAGCAGACACAGCGGCAGAAGCTCACGGCCGAGTTTGAGGAG | |
| | GAGGATCCAAAGTGGGAATTCCCTCGGAAGAACTTGGTTCTTGGAAAAACTC | |
| | TAGGAGAAGGCGAATTTGGAAAAGTGGTCAAGGCAACGGCCTTCCATCTGAA | |
| | AGGCAGAGCAGGGTACACCACGGTGGCCGTGAAGATGCTGAAAGAGAACGCC | |
| | TCCCCGAGTGAGCTGCGAGACCTGCTGTCAGAGTTCAACGTCCTGAAGCAGG | |
| | TCAACCACCCACATGTCATCAAATTGTATGGGGCCTGCAGCCAGGATGGCCC | |
| | GCTCCTCCTCATCGTGGAGTACGCCAAATACGGCTCCCTGCGGGGCTTCCTC | |
| | CGCGAGAGCCGCAAAGTGGGGCCTGGCTACCTGGGCAGTGGAGGCAGCCGCA | |
| | ACTCCAGCTCCCTGGACCACCCGGATGAGCGGGCCCTCACCATGGGCGACCT | |
| | CATCTCATTTGCCTGGCAGATCTCACAGGGGATGCAGTATCTGGCCGAGATG | |
| | AAGCTCGTTCATCGGGACTTGGCAGCCAGAAACATCCTGGTAGCTGAGGGGC | |
| | GGAAGATGAAGATTTCGGATTTCGGCTTGTCCCGAGATGTTTATGAAGAGGA | |
| | TTCCTACGTGAAGAGGAGCCAGGGTCGGATTCCAGTTAAATGGATGGCAATT | |
| | GAATCCCTTTTTGATCATATCTACACCACGCAAAGTGATGTATGGTCTTTTG | |
| | GTGTCCTGCTGTGGGAGATCGTGACCCTAGGGGGAAACCCCTATCCTGGGAT | |
| | TCCTCCTGAGCGGCTCTTCAACCTTCTGAAGACCGGCCACCGGATGGAGAGG | |
| | CCAGACAACTGCAGCGAGGAGATGTACCGCCTGATGCTGCAATGCTGGAAGC | |
| | AGGAGCCGGACAAAAGGCCGGTGTTTGCGGACATCAGCAAAGACCTGGAGAA | |
| | GATGATGGTTAAGAGGAGAGACTACTTGGACCTTGCGGCGTCCACTCCATCT | |
| | GACTCCCTGATTTATGACGACGGCCTCTCAGAGGAGGAGACACCGCTGGTGG | |
| | ACTGTAATAATGCCCCCCTCCCTCGAGCCCTCCCTTCCACATGGATTGAAAA | |
| | CAAACTCTATGGTAGAATTTCCCATGCATTTACTAGATTCTAG | |
| ETV6- | ATGTCTGAGACTCCTGCTCAGTGTAGCATTAAGCAGGAACGAATTTCATATA | 24 |
| RET | CACCTCCAGAGAGCCCAGTGCCGAGTTACGCTTCCTCGACGCCACTTCATGT | |
| | TCCAGTGCCTCGAGCGCTCAGGATGGAGGAAGACTCGATCCGCCTGCCTGCG | |
| | CACCTGCGCTTGCAGCCAATTTACTGGAGCAGGGATGACGTAGCCCAGTGGC | |
| | TCAAGTGGGCTGAAAATGAGTTTTCTTTAAGGCCAATTGACAGCAACACGTT | |
| | TGAAATGAATGGCAAAGCTCTCCTGCTGCTGACCAAAGAGGACTTTCGCTAT | |
| | CGATCTCCTCATTCAGGTGATGTGCTCTATGAACTCCTTCAGCATATTCTGA | |
| | AGCAGAGGAAACCTCGGATTCTTTTTTCACCATTCTTCCACCCTGGAAACTC | |
| | TATACACACACAGCCGGAGGTCATACTGCATCAGAACCATGAAGAAGATAAC | |
| | TGTGTCCAGAGGACCCCCAGGCCATCCGTGGATAATGTGCACCATAACCCTC | |
| | CCACCATTGAACTGTTGCACCGCTCCAGGTCACCTATCACGACAAATCACCG | |
| | GCCTTCTCCTGACCCCGAGCAGCGGCCCCTCCGGTCCCCCCTGGACAACATG | |
| | ATCCGCCGCCTCTCCCCGGCTGAGAGAGCTCAGGGACCCAGGCCGCACCAGG | |
| | AGAACAACCACCAGGAGTCCTACCCTCTGTCAGTGTCTCCCATGGAGAATAA | |
| | TCACTGCCCAGCGTCCTCCGAGTCCCACCCGAAGCCATCCAGCCCCCGGCAG | |
| | GAGAGCACACGCGTGATCCAGCTGATGCCCAGCCCCATCATGCACCCTCTGA | |
| | TCCTGAACCCCCGGCACTCCGTGGATTTCAAACAGTCCAGGCTCTCCGAGGA | |
| | CGGGCTGCATAGGGAAGGGAAGCCCATCAACCTCTCTCATCGGGAAGACCTG | |
| | GCTTACATGAACCACATCATGGTCTCTGTCTCCCCGCCTGAAGAGCACGCCA | |
| | TGCCCATTGGGAGAATAGCAGACTGTAGACTGCTTTGGGATTACGTCTATCA | |
| | GTTGCTTTCTGACAGCCGGTACGAAAACTTCATCCGATGGGAGGACAAAGAA | |
| | TCCAAAATATTCCGGATAGTGGATCCCAACGGACTGGCTCGACTGTGGGGAA | |
| | ACCATAAGGAGGATCCAAAGTGGGAATTCCCTCGGAAGAACTTGGTTCTTGG | |
| | AAAAACTCTAGGAGAAGGCGAATTTGGAAAAGTGGTCAAGGCAACGGCCTTC | |
| | CATCTGAAAGGCAGAGCAGGGTACACCACGGTGGCCGTGAAGATGCTGAAAG | |
| | AGAACGCCTCCCCGAGTGAGCTGCGAGACCTGCTGTCAGAGTTCAACGTCCT | |
| | GAAGCAGGTCAACCACCCACATGTCATCAAATTGTATGGGGCCTGCAGCCAG | |
| | GATGGCCCGCTCCTCCTCATCGTGGAGTACGCCAAATACGGCTCCCTGCGGG | |
| | GCTTCCTCCGCGAGAGCCGCAAAGTGGGGCCTGGCTACCTGGGCAGTGGAGG | |
| | CAGCCGCAACTCCAGCTCCCTGGACCACCCGGATGAGCGGGCCCTCACCATG | |
| | GGCGACCTCATCTCATTTGCCTGGCAGATCTCACAGGGGATGCAGTATCTGG | |
| | CCGAGATGAAGCTCGTTCATCGGGACTTGGCAGCCAGAAACATCCTGGTAGC | |
| | TGAGGGGCGGAAGATGAAGATTTCGGATTTCGGCTTGTCCCGAGATGTTTAT | |
| | GAAGAGGATTCCTACGTGAAGAGGAGCCAGGGTCGGATTCCAGTTAAATGGA | |
| | TGGCAATTGAATCCCTTTTTGATCATATCTACACCACGCAAAGTGATGTATG | |
| | GTCTTTTGGTGTCCTGCTGTGGGAGATCGTGACCCTAGGGGGAAACCCCTAT | |
| | CCTGGGATTCCTCCTGAGCGGCTCTTCAACCTTCTGAAGACCGGCCACCGGA | |
| | TGGAGAGGCCAGACAACTGCAGCGAGGAGATGTACCGCCTGATGCTGCAATG | |
| | CTGGAAGCAGGAGCCGGACAAAAGGCCGGTGTTTGCGGACATCAGCAAAGAC | |
| | CTGGAGAAGATGATGGTTAAGAGGAGAGACTACTTGGACCTTGCGGCGTCCA | |
| | CTCCATCTGACTCCCTGATTTATGACGACGGCCTCTCAGAGGAGGAGACACC | |
| | GCTGGTGGACTGTAATAATGCCCCCCTCCCTCGAGCCCTCCCTTCCACATGG | |
| | ATTGAAAACAAACTCTATGGTAGAATTTCCCATGCATTTACTAGATTCTAG | |
| TAF3- | ATGTGCGAGAGTTACTCCAGGTCGTTGTTGAGGGTCTCGGTGGCGCAGATCT | 25 |
| RET | GCCAGGCGCTGGGCTGGGACTCGGTGCAGCTCAGCGCCTGCCACCTCCTCAC | |
| | GGACGTGCTGCAGCGCTATCTGCAGCAGCTGGGCCGGGGCTGCCATCGGTAC | |
| | TCTGAGCTCTATGGCCGAACAGACCCAATTTTGGATGATGTTGGTGAAGCTT | |
| | TCCAGCTGATGGGGGTTAGTCTACATGAACTAGAAGACTATATTCACAACAT | |
| | TGAGCCTGTCACCTTCCCACACCAAATTCCGTCATTTCCTGTTAGCAAGAAC | |
| | AATGTACTTCAGTTTCCTCAACCTGGAAGTAAAGATGCAGAGGAAAGAAAAG | |
| | AATACATTCCTGATTACCTGCCACCCATTGTGTCTTCTCAAGAAGAAGAAGA | |
| | AGAAGAGCAGGTGCCCACTGATGGAGGCACATCAGCAGAAGCCATGCAGGTT | |
| | CCCTTGGAAGAAGATGATGAATTGGAGGAGGAAGAAATTATTAATGATGAGA | |
| | ATTTCCTGGGCAAGAGACCACTGGATAGTCCTGAAGCTGAAGAACTGCCAGC | |
| | CATGAAGCGGCCTCGGCTATTAAGCACTAAAGGGGACACGCTAGATGTTGTG | |
| | TTATTGGAAGCTCGAGAGCCACTCAGCTCAATAAATACTCAAAAGATCCCAC | |
| | CAATGCTTTCTCCAGTCCATGTACAGGACAGTACAGACTTGGCACCTCCCTC | |
| | ACCCGAGCCGCCAATGTTGGCTCCAGTTGCAAAATCACAAATGCCAACTGCA | |
| | AAACCATTAGAAACAAAGTCATTTACACCTAAAACAAAGACTAAAACTAGCT | |
| | CTCCAGGACAGAAGACTAAATCACCTAAAACCGCCCAGTCACCAGCAATGGT | |
| | CGGAAGTCCTATTCGATCACCAAAAACTGTATCCAAAGAAAAGAAATCACCT | |
| | GGACGTTCCAAGAGCCCCAAGAGTCCCAAGAGCCCCAAGGTCACGACTCACA | |
| | TTCCCCAAACACCTGTGAGACCTGAAACGCCCAACAGGACTCCTTCAGCTAC | |
| | ACTCAGTGAAAAAATCAGTAAAGAGACTATCCAGGTAAAACAAATACAGACA | |
| | CCCCCTGATGCTGGGAAACTGAACAGTGAGAATCAGCCGAAAAAGGCTGTGG | |
| | TAGCAGATAAAACGATTGAGGCCTCTATCGATGCTGTGATTGCACGAGCCTG | |
| | TGCTGAGCGAGAGCCAGATCCTTTCGAATTTTCTTCTGGATCGGAATCTGAA | |
| | GGAGACATTTTTACTAGCCCTAAGAGAATTTCAGGCCCGGAGTGTACTACTC | |
| | CCAAAGCTTCCACTTCCGCGAACAATTTCACAAAGTCAGGATCCACTCCTCT | |
| | GCCTCTTTCCGGTGGAACCTCAAGTTCCGATAACTCATGGACAATGGATGCC | |
| | TCCATTGATGAGGTTGTACGTAAAGCAAAACTGGGAACACCTTCAAATATGC | |
| | CCCCCAACTTTCCTTATATCTCTTCTCCGTCAGTGTCTCCTCCCACTCCCGA | |
| | ACCTCTCCACAAGGTGTATGAGGAGAAAACCAAGCTGCCTTCCTCCGTGGAG | |
| | GTAAAGAAGAAGTTGAAAAAGGAACTAAAGACTAAAATGAAAAAGAAAGAAA | |
| | AGCAGAGAGATAGGGAGAGGGAAAAAGACAAGAACAAGGACAAAAGTAAGGA | |
| | GAAGGATAAAGTGAAAGAGAAAGAGAAAGACAAGGAAACTGGCAGGGAAACA | |
| | AAGTATCCCTGGAAGGAATTTCTTAAAGAGGAAGAGGCAGATCCCTACAAGT | |
| | TTAAAATCAAAGAATTTGAAGATGTTGATCCCAAAGTGAAATTGAAAGATGG | |
| | ACTTGTGAGGAAGGAGAAAGAGAAGCATAAAGATAAGAAGAAAGATAGAGAG | |
| | AAAGGCAAGAAAGATAAAGATAAGAGAGAGAAAGAAAAAGTGAAAGATAAAG | |
| | GCAGAGAAGATAAGATGAAAGCCCCAGCACCCCCACTGGTGTTGCCCCCAAA | |
| | AGAGTTGGCCCTGCCCTTGTTCAGCCCTGCCACAGCCTCCAGGGTCCCAGCC | |
| | ATGCTGCCATCTTTGTTGCCAGTGCTTCCGGAAAAACTGTTTGAGGAGAAAG | |
| | AGAAGGTGAAGGAGAAAGAAAAGAAAAAGGACAAAAAGGAGAAGAAGAAAAA | |
| | GAAGGAAAAAGAGAAGGAGAAGAAGGAGAAGGAAAGAGAGAAAGAGAAGAGA | |
| | GAGCGAGAGAAGAGAGAAAAAGAGAAGGAGAAACACAAGCATGAAAAAGAGG | |
| | ATCCAAAGTGGGAATTCCCTCGGAAGAACTTGGTTCTTGGAAAAACTCTAGG | |
| | AGAAGGCGAATTTGGAAAAGTGGTCAAGGCAACGGCCTTCCATCTGAAAGGC | |
| | AGAGCAGGGTACACCACGGTGGCCGTGAAGATGCTGAAAGAGAACGCCTCCC | |
| | CGAGTGAGCTGCGAGACCTGCTGTCAGAGTTCAACGTCCTGAAGCAGGTCAA | |
| | CCACCCACATGTCATCAAATTGTATGGGGCCTGCAGCCAGGATGGCCCGCTC | |
| | CTCCTCATCGTGGAGTACGCCAAATACGGCTCCCTGCGGGGCTTCCTCCGCG | |
| | AGAGCCGCAAAGTGGGGCCTGGCTACCTGGGCAGTGGAGGCAGCCGCAACTC | |
| | CAGCTCCCTGGACCACCCGGATGAGCGGGCCCTCACCATGGGCGACCTCATC | |
| | TCATTTGCCTGGCAGATCTCACAGGGGATGCAGTATCTGGCCGAGATGAAGC | |
| | TCGTTCATCGGGACTTGGCAGCCAGAAACATCCTGGTAGCTGAGGGGCGGAA | |
| | GATGAAGATTTCGGATTTCGGCTTGTCCCGAGATGTTTATGAAGAGGATTCC | |
| | TACGTGAAGAGGAGCCAGGGTCGGATTCCAGTTAAATGGATGGCAATTGAAT | |
| | CCCTTTTTGATCATATCTACACCACGCAAAGTGATGTATGGTCTTTTGGTGT | |
| | CCTGCTGTGGGAGATCGTGACCCTAGGGGGAAACCCCTATCCTGGGATTCCT | |
| | CCTGAGCGGCTCTTCAACCTTCTGAAGACCGGCCACCGGATGGAGAGGCCAG | |
| | ACAACTGCAGCGAGGAGATGTACCGCCTGATGCTGCAATGCTGGAAGCAGGA | |
| | GCCGGACAAAAGGCCGGTGTTTGCGGACATCAGCAAAGACCTGGAGAAGATG | |
| | ATGGTTAAGAGGAGAGACTACTTGGACCTTGCGGCGTCCACTCCATCTGACT | |
| | CCCTGATTTATGACGACGGCCTCTCAGAGGAGGAGACACCGCTGGTGGACTG | |
| | TAATAATGCCCCCCTCCCTCGAGCCCTCCCTTCCACATGGATTGAAAACAAA | |
| | CTCTATGGTAGAATTTCCCATGCATTTACTAGATTCTAG | |
| PCM1- | ATGGCCACAGGAGGAGGTCCCTTTGAAGATGGCATGAATGATCAGGATTTAC | 26 |
| RET | CAAACTGGAGTAATGAGAATGTTGATGACAGGCTCAACAATATGGATTGGGG | |
| | TGCCCAACAGAAGAAAGCAAATAGATCATCAGAAAAGAATAAGAAAAAGTTT | |
| | GGTGTAGAAAGTGATAAAAGAGTAACCAATGATATTTCTCCGGAGTCGTCAC | |
| | CAGGAGTTGGAAGGCGAAGAACAAAGACTCCACATACGTTCCCACACAGTAG | |
| | ATACATGAGTCAGATGTCTGTCCCAGAGCAGGCAGAATTAGAGAAACTGAAA | |
| | CAGCGGATAAACTTCAGTGATTTAGATCAGAGAAGCATTGGAAGTGATTCCC | |
| | AAGGTAGAGCAACAGCTGCTAACAACAAACGTCAGCTTAGTGAAAACCGAAA | |
| | GCCCTTCAACTTTTTGCCTATGCAGATTAATACTAACAAGAGCAAAGATGCA | |
| | TCTACAAACCCCCCAAACAGAGAAACGATTGGATCAGCACAGTGTAAAGAGT | |
| | TGTTTGCTTCTGCTTTAAGTAATGACCTCTTGCAAAACTGTCAGGTGTCTGA | |
| | AGAAGATGGGAGGGGAGAACCTGCAATGGAGAGCAGCCAGATTGTAAGCAGG | |
| | CTTGTTCAAATTCGCGATTATATTACTAAAGCTAGTTCCATGCGGGAAGATC | |
| | TTGTAGAGAAAAATGAGAGATCTGCTAATGTTGAGCGCCTTACTCATCTAAT | |
| | AGATCACCTTAAAGAACAAGAGAAGTCATATATGAAATTTCTTAAAAAAATC | |
| | CTTGCCAGAGATCCTCAGCAGGAGCCTATGGAAGAGATAGAAAATTTGAAGA | |
| | AACAACATGATTTATTAAAAAGAATGTTACAACAGCAGGAGCAACTAAGAGC | |
| | TCTACAGGGACGGCAGGCTGCACTTCTAGCTCTGCAACATAAAGCAGAGCAA | |
| | GCTATTGCAGTGATGGATGATTCTGTTGTTGCAGAAACTGCAGGTAGCTTAT | |
| | CTGGCGTCAGTATCACATCTGAACTAAATGAAGAATTGAATGACTTAATTCA | |
| | GCGTTTTCATAATCAGCTTCGTGATTCTCAGCCTCCAGCTGTTCCAGACAAT | |
| | AGAAGACAGGCAGAAAGTCTTTCATTAACTAGGGAGGTTTCCCAGAGCAGGA | |
| | AACCATCAGCTTCAGAACGTTTACCTGATGAGAAAGTCGAACTTTTTAGCAA | |
| | AATGAGAGTGCTACAGGAAAAGAAACAAAAAATGGACAAATTGCTTGGAGAA | |
| | CTTCATACACTTCGAGATCAGCATCTTAACAATTCATCATCCTCTCCACAAA | |
| | GGAGTGTCGATCAGAGAAGTACTTCAGCTCCCTCTGCTTCTGTAGGCTTGGC | |
| | ACCGGTTGTCAATGGAGAATCCAATAGCCTCACATCATCTGTTCCTTATCCT | |
| | ACTGCTTCTCTAGTATCTCAGAATGAGAGTGAAAACGAAGGCCACCTCAATC | |
| | CATCTGAAAAACTCCAGAAGTTAAATGAAGTTCGAAAGAGATTGAATGAGCT | |
| | AAGAGAATTAGTTCATTATTATGAACAAACGTCAGACATGATGACAGATGCT | |
| | GTGAATGAAAACAGGAAAGATGAAGAAACTGAAGAGTCAGAATATGATTCTG | |
| | AGCATGAAAATTCCGAGCCTGTTACTAACATTCGAAATCCACAAGTAGCTTC | |
| | CACTTGGAATGAAGTAAATAGTCATAGTAATGCACAGTGTGTTTCTAATAAT | |
| | AGAGATGGGCGAACAGTTAATTCTAATTGTGAAATTAACAACAGATCTGCTG | |
| | CCAACATAAGGGCTCTAAACATGCCTCCTTCTTTAGATTGTCGATATAATAG | |
| | AGAAGGGGAACAGGAGATTCATGTTGCACAAGGTGAAGATGATGAGGAGGAG | |
| | GAGGAAGAAGCAGAAGAGGAGGGAGTCAGTGGAGCTTCATTATCTAGTCACA | |
| | GGAGCAGTCTGGTTGATGAGCATCCAGAAGATGCTGAATTTGAACAGAAGAT | |
| | CAACCGACTTATGGCTGCAAAACAGAAACTTAGACAGTTACAAGATCTTGTT | |
| | GCTATGGTACAGGATGATGATGCAGCTCAAGGAGTTATCTCTGCCAGTGCAT | |
| | CAAATTTGGATGATTTCTACCCAGCAGAAGAAGACACCAAGCAAAATTCAAA | |
| | TAACACTAGAGGAAATGCCAATAAAACACAGAAAGATACTGGAGTAAATGAA | |
| | AAGGCAAGAGAGAAATTTTATGAGGCTAAACTACAGCAGCAACAGAGAGAGC | |
| | TAAAACAATTGCAGGAAGAAAGAAAGAAACTGATTGACATTCAGGAGAAAAT | |
| | TCAAGCATTGCAAACGGCATGCCCTGACTTACAGCTGTCAGCTGCTAGTGTG | |
| | GGTAACTGTCCCACCAAAAAATATATGCCAGCTGTTACTTCAACCCCAACTG | |
| | TTAATCAACACGAGACCAGTACAAGCAAATCTGTTTTTGAGCCTGAAGATTC | |
| | TTCAATAGTAGATAATGAGTTGTGGTCAGAAATGAGAAGACATGAAATGTTG | |
| | AGGGAGGAGCTGCGACAGAGAAGAAAGCAGCTTGAAGCTCTGATGGCTGAAC | |
| | ATCAGAGGAGGCAAGGTCTAGCTGAAACTGCATCTCCAGTGGCTGTGTCATT | |
| | GAGAAGTGATGGATCTGAGAACCTATGTACTCCTCAGCAAAGTAGAACAGAA | |
| | AAAACGATGGCAACTTGGGGAGGGTCTACCCAGTGTGCACTAGATGAAGAAG | |
| | GAGATGAAGACGGTTACCTTTCTGAAGGAATTGTTCGGACAGATGAAGAGGA | |
| | GGAAGAAGAGCAAGATGCCAGTTCCAATGATAACTTTTCTGTGTGTCCTTCT | |
| | AACAGTGTGAATCATAACTCCTACAATGGAAAGGAAACTAAAAATAGGTGGA | |
| | AGAACAATTGCCCTTTTTCGGCAGATGAAAATTATCGTCCTTTAGCCAAGAC | |
| | AAGGCAACAGAATATCAGCATGCAACGGCAAGAAAACCTTCGTTGGGTGTCA | |
| | GAGCTCTCTTACGTAGAAGAGAAAGAACAATGGCAAGAACAAATCAATCAGC | |
| | TAAAGAAACAGCTTGATTTTAGTGTCAGTATTTGTCAGACTTTGATGCAAGA | |
| | CCAGCAGACTCTATCTTGTCTGCTACAAACTCTTCTCACGGGTCCTTACAGT | |
| | GTTATGCCCAGCAATGTTGCATCTCCTCAAGTACACTTCATAATGCACCAGT | |
| | TGAACCAGTGCTATACTCAGCTAACATGGCAACAGAATAATGTTCAGAGGTT | |
| | GAAACAAATGCTAAATGAACTTATGCGCCAGCAAAATCAGCATCCAGAAAAA | |
| | CCTGGAGGCAAGGAAAGAGGCAGTAGTGCATCGCACCCTCCTTCTCCCAGTT | |
| | TATTTTGTCCTTTCAGCTTTCCAACACAGCCTGTAAATCTCTTCAATATACC | |
| | TGGATTTACTAACTTTTCATCATTTGCACCAGGTATGAATTTCAGCCCTTTA | |
| | TTTCCTTCTAATTTTGGAGATTTTTCTCAGAATATCTCTACACCCAGTGAAC | |
| | AGCAGCAACCCTTAGCCCAGAATTCTTCAGGAAAAACAGAATATATGGCTTT | |
| | TCCAAAACCTTTTGAAAGCAGTTCCTCTATTGGAGCAGAGAAACCAAGGAAT | |
| | AAAAAACTGCCTGAAGAGGAGGTGGAAAGCAGTAGGACACCATGGTTATATG | |
| | AACAAGAAGGTGAAGTAGAGAAACCATTTATCAAGACTGGATTTTCAGTGTC | |
| | TGTAGAAAAATCTACAAGTAGTAACCGCAAAAATCAATTAGATACAAACGGA | |
| | AGAAGACGCCAGTTTGATGAAGAATCACTGGAAAGCTTTAGCAGTATGCCTG | |
| | ATCCAGTAGATCCAACAACAGTGACTAAAACATTCAAGACAAGAAAAGCGTC | |
| | TGCACAGGCCAGCCTGGCATCTAAAGATAAAACTCCCAAGTCAAAAAGTAAG | |
| | AAGAGGAATTCTACTCAGCTGAAAAGCAGAGTTAAAAACATCAGGTATGAAA | |
| | GTGCCAGTATGTCTAGCACATGTGAACCTTGCAAAAGTAGGAACAGACATTC | |
| | AGCCCAGACTGAAGAGCCTGTTCAAGCAAAAGTATTCAGCAGAAAGAATCAT | |
| | GAGCAACTGGAAAAAATAATAAAATGTAATAGGTCTACAGAAATATCTTCAG | |
| | AAACTGGGAGTGATTTTTCCATGTTTGAAGCTTTGCGAGATACTATTTATTC | |
| | TGAAGTAGCTACATTAATTTCTCAAAATGAATCTCGTCCACATTTTCTTATT | |
| | GAACTCTTCCATGAGCTGCAGCTACTAAACACAGACTACTTGAGACAGAGGG | |
| | CTTTATATGCATTGCAGGACATAGTATCCAGACATATTTCTGAGAGCCATGA | |
| | AAAAGGAGAAAATGTAAAGTCAGTAAACTCTGGTACTTGGATAGCATCAAAC | |
| | TCAGAACTTACTCCTAGTGAGAGCCTTGCTACTACTGATGATGAAACTTTTG | |
| | AGAAGAACTTTGAAAGAGAAACCCATAAAATAAGTGAGCAAAATGATGCTGA | |
| | TAATGCTAGTGTCCTGTCTGTATCATCAAATTTTGAGCCTTTTGCAACAGAT | |
| | GATCTAGGTAACACCGTGATTCACTTAGATCAAGCATTAGCCAGAATGAGAG | |
| | AATATGAGCGTATGAAGACTGAGGCTGAAAGTAACTCAAATATGAGATGCAC | |
| | CTGCAGGATTATTGAGGATGGAGATGGTGCTGGTGCAGGTACTACAGTTAAT | |
| | AATTTAGAAGAAACTCCCGTTATTGAAAATCGTAGTTCACAACAACCTGTAA | |
| | GTGAAGTTTCTACCATCCCATGTCCTAGAATTGATACTCAGCAGCTGGACCG | |
| | GCAAATTAAAGCAATTATGAAAGAAGTCATTCCTTTTTTGAAGGAGGATCCA | |
| | AAGTGGGAATTCCCTCGGAAGAACTTGGTTCTTGGAAAAACTCTAGGAGAAG | |
| | GCGAATTTGGAAAAGTGGTCAAGGCAACGGCCTTCCATCTGAAAGGCAGAGC | |
| | AGGGTACACCACGGTGGCCGTGAAGATGCTGAAAGAGAACGCCTCCCCGAGT | |
| | GAGCTGCGAGACCTGCTGTCAGAGTTCAACGTCCTGAAGCAGGTCAACCACC | |
| | CACATGTCATCAAATTGTATGGGGCCTGCAGCCAGGATGGCCCGCTCCTCCT | |
| | CATCGTGGAGTACGCCAAATACGGCTCCCTGCGGGGCTTCCTCCGCGAGAGC | |
| | CGCAAAGTGGGGCCTGGCTACCTGGGCAGTGGAGGCAGCCGCAACTCCAGCT | |
| | CCCTGGACCACCCGGATGAGCGGGCCCTCACCATGGGCGACCTCATCTCATT | |
| | TGCCTGGCAGATCTCACAGGGGATGCAGTATCTGGCCGAGATGAAGCTCGTT | |
| | CATCGGGACTTGGCAGCCAGAAACATCCTGGTAGCTGAGGGGCGGAAGATGA | |
| | AGATTTCGGATTTCGGCTTGTCCCGAGATGTTTATGAAGAGGATTCCTACGT | |
| | GAAGAGGAGCCAGGGTCGGATTCCAGTTAAATGGATGGCAATTGAATCCCTT | |
| | TTTGATCATATCTACACCACGCAAAGTGATGTATGGTCTTTTGGTGTCCTGC | |
| | TGTGGGAGATCGTGACCCTAGGGGGAAACCCCTATCCTGGGATTCCTCCTGA | |
| | GCGGCTCTTCAACCTTCTGAAGACCGGCCACCGGATGGAGAGGCCAGACAAC | |
| | TGCAGCGAGGAGATGTACCGCCTGATGCTGCAATGCTGGAAGCAGGAGCCGG | |
| | ACAAAAGGCCGGTGTTTGCGGACATCAGCAAAGACCTGGAGAAGATGATGGT | |
| | TAAGAGGAGAGACTACTTGGACCTTGCGGCGTCCACTCCATCTGACTCCCTG | |
| | ATTTATGACGACGGCCTCTCAGAGGAGGAGACACCGCTGGTGGACTGTAATA | |
| | ATGCCCCCCTCCCTCGAGCCCTCCCTTCCACATGGATTGAAAACAAACTCTA | |
| | TGGTAGAATTTCCCATGCATTTACTAGATTCTAG | |
| PCM1- | ATGGCCACAGGAGGAGGTCCCTTTGAAGATGGCATGAATGATCAGGATTTAC | 27 |
| RET | CAAACTGGAGTAATGAGAATGTTGATGACAGGCTCAACAATATGGATTGGGG | |
| | TGCCCAACAGAAGAAAGCAAATAGATCATCAGAAAAGAATAAGAAAAAGTTT | |
| | GGTGTAGAAAGTGATAAAAGAGTAACCAATGATATTTCTCCGGAGTCGTCAC | |
| | CAGGAGTTGGAAGGCGAAGAACAAAGACTCCACATACGTTCCCACACAGTAG | |
| | ATACATGAGTCAGATGTCTGTCCCAGAGCAGGCAGAATTAGAGAAACTGAAA | |
| | CAGCGGATAAACTTCAGTGATTTAGATCAGAGAAGCATTGGAAGTGATTCCC | |
| | AAGGTAGAGCAACAGCTGCTAACAACAAACGTCAGCTTAGTGAAAACCGAAA | |
| | GCCCTTCAACTTTTTGCCTATGCAGATTAATACTAACAAGAGCAAAGATGCA | |
| | TCTACAAACCCCCCAAACAGAGAAACGATTGGATCAGCACAGTGTAAAGAGT | |
| | TGTTTGCTTCTGCTTTAAGTAATGACCTCTTGCAAAACTGTCAGGTGTCTGA | |
| | AGAAGATGGGAGGGGAGAACCTGCAATGGAGAGCAGCCAGATTGTAAGCAGG | |
| | CTTGTTCAAATTCGCGATTATATTACTAAAGCTAGTTCCATGCGGGAAGATC | |
| | TTGTAGAGAAAAATGAGAGATCTGCTAATGTTGAGCGCCTTACTCATCTAAT | |
| | AGATCACCTTAAAGAACAAGAGAAGTCATATATGAAATTTCTTAAAAAAATC | |
| | CTTGCCAGAGATCCTCAGCAGGAGCCTATGGAAGAGATAGAAAATTTGAAGA | |
| | AACAACATGATTTATTAAAAAGAATGTTACAACAGCAGGAGCAACTAAGAGC | |
| | TCTACAGGGACGGCAGGCTGCACTTCTAGCTCTGCAACATAAAGCAGAGCAA | |
| | GCTATTGCAGTGATGGATGATTCTGTTGTTGCAGAAACTGCAGGTAGCTTAT | |
| | CTGGCGTCAGTATCACATCTGAACTAAATGAAGAATTGAATGACTTAATTCA | |
| | GCGTTTTCATAATCAGCTTCGTGATTCTCAGCCTCCAGCTGTTCCAGACAAT | |
| | AGAAGACAGGCAGAAAGTCTTTCATTAACTAGGGAGGTTTCCCAGAGCAGGA | |
| | AACCATCAGCTTCAGAACGTTTACCTGATGAGAAAGTCGAACTTTTTAGCAA | |
| | AATGAGAGTGCTACAGGAAAAGAAACAAAAAATGGACAAATTGCTTGGAGAA | |
| | CTTCATACACTTCGAGATCAGCATCTTAACAATTCATCATCCTCTCCACAAA | |
| | GGAGTGTCGATCAGAGAAGTACTTCAGCTCCCTCTGCTTCTGTAGGCTTGGC | |
| | ACCGGTTGTCAATGGAGAATCCAATAGCCTCACATCATCTGTTCCTTATCCT | |
| | ACTGCTTCTCTAGTATCTCAGAATGAGAGTGAAAACGAAGGCCACCTCAATC | |
| | CATCTGAAAAACTCCAGAAGTTAAATGAAGTTCGAAAGAGATTGAATGAGCT | |
| | AAGAGAATTAGTTCATTATTATGAACAAACGTCAGACATGATGACAGATGCT | |
| | GTGAATGAAAACAGGAAAGATGAAGAAACTGAAGAGTCAGAATATGATTCTG | |
| | AGCATGAAAATTCCGAGCCTGTTACTAACATTCGAAATCCACAAGTAGCTTC | |
| | CACTTGGAATGAAGTAAATAGTCATAGTAATGCACAGTGTGTTTCTAATAAT | |
| | AGAGATGGGCGAACAGTTAATTCTAATTGTGAAATTAACAACAGATCTGCTG | |
| | CCAACATAAGGGCTCTAAACATGCCTCCTTCTTTAGATTGTCGATATAATAG | |
| | AGAAGGGGAACAGGAGATTCATGTTGCACAAGGTGAAGATGATGAGGAGGAG | |
| | GAGGAAGAAGCAGAAGAGGAGGGAGTCAGTGGAGCTTCATTATCTAGTCACA | |
| | GGAGCAGTCTGGTTGATGAGCATCCAGAAGATGCTGAATTTGAACAGAAGAT | |
| | CAACCGACTTATGGCTGCAAAACAGAAACTTAGACAGTTACAAGATCTTGTT | |
| | GCTATGGTACAGGATGATGATGCAGCTCAAGGAGTTATCTCTGCCAGTGCAT | |
| | CAAATTTGGATGATTTCTACCCAGCAGAAGAAGACACCAAGCAAAATTCAAA | |
| | TAACACTAGAGGAAATGCCAATAAAACACAGAAAGATACTGGAGTAAATGAA | |
| | AAGGCAAGAGAGAAATTTTATGAGGCTAAACTACAGCAGCAACAGAGAGAGC | |
| | TAAAACAATTGCAGGAAGAAAGAAAGAAACTGATTGACATTCAGGAGAAAAT | |
| | TCAAGCATTGCAAACGGCATGCCCTGACTTACAGCTGTCAGCTGCTAGTGTG | |
| | GGTAACTGTCCCACCAAAAAATATATGCCAGCTGTTACTTCAACCCCAACTG | |
| | TTAATCAACACGAGACCAGTACAAGCAAATCTGTTTTTGAGCCTGAAGATTC | |
| | TTCAATAGTAGATAATGAGTTGTGGTCAGAAATGAGAAGACATGAAATGTTG | |
| | AGGGAGGAGCTGCGACAGAGAAGAAAGCAGCTTGAAGCTCTGATGGCTGAAC | |
| | ATCAGAGGAGGCAAGGTCTAGCTGAAACTGCATCTCCAGTGGCTGTGTCATT | |
| | GAGAAGTGATGGATCTGAGAACCTATGTACTCCTCAGCAAAGTAGAACAGAA | |
| | AAAACGATGGCAACTTGGGGAGGGTCTACCCAGTGTGCACTAGATGAAGAAG | |
| | GAGATGAAGACGGTTACCTTTCTGAAGGAATTGTTCGGACAGATGAAGAGGA | |
| | GGAAGAAGAGCAAGATGCCAGTTCCAATGATAACTTTTCTGTGTGTCCTTCT | |
| | AACAGTGTGAATCATAACTCCTACAATGGAAAGGAAACTAAAAATAGGTGGA | |
| | AGAACAATTGCCCTTTTTCGGCAGATGAAAATTATCGTCCTTTAGCCAAGAC | |
| | AAGGCAACAGAATATCAGCATGCAACGGCAAGAAAACCTTCGTTGGGTGTCA | |
| | GAGCTCTCTTACGTAGAAGAGAAAGAACAATGGCAAGAACAAATCAATCAGC | |
| | TAAAGAAACAGCTTGATTTTAGTGTCAGTATTTGTCAGACTTTGATGCAAGA | |
| | CCAGCAGACTCTATCTTGTCTGCTACAAACTCTTCTCACGGGTCCTTACAGT | |
| | GTTATGCCCAGCAATGTTGCATCTCCTCAAGTACACTTCATAATGCACCAGT | |
| | TGAACCAGTGCTATACTCAGCTAACATGGCAACAGAATAATGTTCAGAGGTT | |
| | GAAACAAATGCTAAATGAACTTATGCGCCAGCAAAATCAGCATCCAGAAAAA | |
| | CCTGGAGGCAAGGAAAGAGGCAGTAGTGCATCGCACCCTCCTTCTCCCAGTT | |
| | TATTTTGTCCTTTCAGCTTTCCAACACAGCCTGTAAATCTCTTCAATATACC | |
| | TGGATTTACTAACTTTTCATCATTTGCACCAGGTATGAATTTCAGCCCTTTA | |
| | TTTCCTTCTAATTTTGGAGATTTTTCTCAGAATATCTCTACACCCAGTGAAC | |
| | AGCAGCAACCCTTAGCCCAGAATTCTTCAGGAAAAACAGAATATATGGCTTT | |
| | TCCAAAACCTTTTGAAAGCAGTTCCTCTATTGGAGCAGAGAAACCAAGGAAT | |
| | AAAAAACTGCCTGAAGAGGAGGTGGAAAGCAGTAGGACACCATGGTTATATG | |
| | AACAAGAAGGTGAAGTAGAGAAACCATTTATCAAGACTGGATTTTCAGTGTC | |
| | TGTAGAAAAATCTACAAGTAGTAACCGCAAAAATCAATTAGATACAAACGGA | |
| | AGAAGACGCCAGTTTGATGAAGAATCACTGGAAAGCTTTAGCAGTATGCCTG | |
| | ATCCAGTAGATCCAACAACAGTGACTAAAACATTCAAGACAAGAAAAGCGTC | |
| | TGCACAGGCCAGCCTGGCATCTAAAGATAAAACTCCCAAGTCAAAAAGTAAG | |
| | AAGAGGAATTCTACTCAGCTGAAAAGCAGAGTTAAAAACATCAGGTATGAAA | |
| | GTGCCAGTATGTCTAGCACATGTGAACCTTGCAAAAGTAGGAACAGACATTC | |
| | AGCCCAGACTGAAGAGCCTGTTCAAGCAAAAGTATTCAGCAGAAAGAATCAT | |
| | GAGCAACTGGAAAAAATAATAAAATGTAATAGGTCTACAGAAATATCTTCAG | |
| | AAACTGGGAGTGATTTTTCCATGTTTGAAGCTTTGCGAGATACTATTTATTC | |
| | TGAAGTAGCTACATTAATTTCTCAAAATGAATCTCGTCCACATTTTCTTATT | |
| | GAACTCTTCCATGAGCTGCAGCTACTAAACACAGACTACTTGAGACAGAGGG | |
| | CTTTATATGCATTGCAGGACATAGTATCCAGACATATTTCTGAGAGCCATGA | |
| | AAAAGGAGAAAATGTAAAGTCAGTAAACTCTGGTACTTGGATAGCATCAAAC | |
| | TCAGAACTTACTCCTAGTGAGAGCCTTGCTACTACTGATGATGAAACTTTTG | |
| | AGAAGAACTTTGAAAGAGAAACCCATAAAATAAGTGAGCAAAATGATGCTGA | |
| | TAATGCTAGTGTCCTGTCTGTATCATCAAATTTTGAGCCTTTTGCAACAGAT | |
| | GATCTAGGTAACACCGTGATTCACTTAGATCAAGCATTAGCCAGAATGAGAG | |
| | AATATGAGCGTATGAAGACTGAGGCTGAAAGTAACTCAAATATGAGATGCAC | |
| | CTGCAGGATTATTGAGGATGGAGATGGTGCTGGTGCAGGTACTACAGTTAAT | |
| | AATTTAGAAGAAACTCCCGTTATTGAAAATCGTAGTTCACAACAACCTGTAA | |
| | GTGAAGTTTCTACCATCCCATGTCCTAGAATTGATACTCAGCAGCTGGACCG | |
| | GCAAATTAAAGCAATTATGAAAGAAGTCATTCCTTTTTTGAAGGAGGATCCA | |
| | AAGTGGGAATTCCCTCGGAAGAACTTGGTTCTTGGAAAAACTCTAGGAGAAG | |
| | GCGAATTTGGAAAAGTGGTCAAGGCAACGGCCTTCCATCTGAAAGGCAGAGC | |
| | AGGGTACACCACGGTGGCCGTGAAGATGCTGAAAGAGAACGCCTCCCCGAGT | |
| | GAGCTGCGAGACCTGCTGTCAGAGTTCAACGTCCTGAAGCAGGTCAACCACC | |
| | CACATGTCATCAAATTGTATGGGGCCTGCAGCCAGGATGGCCCGCTCCTCCT | |
| | CATCGTGGAGTACGCCAAATACGGCTCCCTGCGGGGCTTCCTCCGCGAGAGC | |
| | CGCAAAGTGGGGCCTGGCTACCTGGGCAGTGGAGGCAGCCGCAACTCCAGCT | |
| | CCCTGGACCACCCGGATGAGCGGGCCCTCACCATGGGCGACCTCATCTCATT | |
| | TGCCTGGCAGATCTCACAGGGGATGCAGTATCTGGCCGAGATGAAGCTCGTT | |
| | CATCGGGACTTGGCAGCCAGAAACATCCTGGTAGCTGAGGGGCGGAAGATGA | |
| | AGATTTCGGATTTCGGCTTGTCCCGAGATGTTTATGAAGAGGATTCCTACGT | |
| | GAAGAGGAGCCAGGGTCGGATTCCAGTTAAATGGATGGCAATTGAATCCCTT | |
| | TTTGATCATATCTACACCACGCAAAGTGATGTATGGTCTTTTGGTGTCCTGC | |
| | TGTGGGAGATCGTGACCCTAGGGGGAAACCCCTATCCTGGGATTCCTCCTGA | |
| | GCGGCTCTTCAACCTTCTGAAGACCGGCCACCGGATGGAGAGGCCAGACAAC | |
| | TGCAGCGAGGAGATGTACCGCCTGATGCTGCAATGCTGGAAGCAGGAGCCGG | |
| | ACAAAAGGCCGGTGTTTGCGGACATCAGCAAAGACCTGGAGAAGATGATGGT | |
| | TAAGAGGAGAGACTACTTGGACCTTGCGGCGTCCACTCCATCTGACTCCCTG | |
| | ATTTATGACGACGGCCTCTCAGAGGAGGAGACACCGCTGGTGGACTGTAATA | |
| | ATGCCCCCCTCCCTCGAGCCCTCCCTTCCACATGGATTGAAAACAAACTCTA | |
| | TGGTAGAATTTCCCATGCATTTACTAGATTCTAG | |
| TNIP2- | ATGTCCCGGGACCCGGGGTCGGGCGGCTGGGAGGAGGCCCCGCGCGCAGCTG | 28 |
| RET | CCGCGCTCTGCACCCTGTACCACGAGGCCGGACAGCGGCTGCGCCGCCTGCA | |
| | GGACCAGCTCGCTGCCCGCGACGCCCTCATCGCTCGCCTCCGCGCCCGCCTG | |
| | GCCGCGCTGGAGGGGGACGCCGCGCCGTCCCTAGTGGACGCGCTGCTGGAGC | |
| | AGGTTGCGCGCTTCCGGGAGCAGCTGCGAAGGCAGGAGGGCGGCGCCGCCGA | |
| | GGCCCAGATGCGCCAGGAAATTGAGAGGCTGACTGAGCGACTAGAAGAAAAA | |
| | GAGAGGGAGATGCAGCAGCTGCTGAGCCAGCCCCAACACGAGCGAGAGAAGG | |
| | AAGTCGTCCTGCTACGGAGGAGCATGGCAGAAGGGGAGCGCGCCCGGGCCGC | |
| | CAGTGACGTCCTGTGCCGCTCCTTGGCCAACGAGACCCATCAGCTGCGGAGG | |
| | ACGCTGACCGCCACCGCCCACATGTGTCAGCATCTGGCCAAGTGTCTGGATG | |
| | AACGACAGCATGCACAAAGGAATGTGGGGGAGAGAAGTCCTGACCAGTCGGA | |
| | ACACACAGATGGGCACACCTCTGTCCAGAGTGTTATTGAGAAGTTGCAGGAA | |
| | GAAAATCGACTGTTAAAACAGAAGGTGACTCACGTTGAAGACCTCAATGCCA | |
| | AGTGGCAGCGCTACAACGCCAGCAGGGACGAATACGTGAGGGGGCTCCATGC | |
| | GCAGCTCAGGGGGCTGCAGATCCCCCACGAGCCCGAGCTGATGAGGAAGGAG | |
| | ATCTCCCGGCTCAACAGACAGTTGGAAGAGAAAATAAATGACTGTGCCGAAG | |
| | TGAAGCAGGAGCTGGCGGCCTCCAGGACGGCCCGGGATGCTGCGTTGGAGCG | |
| | GGTGCAGATGCTGGAACAGCAGATTCTCGCTTACAAGGATGACTTCATGTCA | |
| | GAAAGGGCCGATCGGGAACGGGCTCAAAGTAGGATTCAAGAACTGGAGGAAA | |
| | AGGTCGCCTCTTTGCTGCACCAGGTGTCCTGGAGACAGGAGGATCCAAAGTG | |
| | GGAATTCCCTCGGAAGAACTTGGTTCTTGGAAAAACTCTAGGAGAAGGCGAA | |
| | TTTGGAAAAGTGGTCAAGGCAACGGCCTTCCATCTGAAAGGCAGAGCAGGGT | |
| | ACACCACGGTGGCCGTGAAGATGCTGAAAGAGAACGCCTCCCCGAGTGAGCT | |
| | GCGAGACCTGCTGTCAGAGTTCAACGTCCTGAAGCAGGTCAACCACCCACAT | |
| | GTCATCAAATTGTATGGGGCCTGCAGCCAGGATGGCCCGCTCCTCCTCATCG | |
| | TGGAGTACGCCAAATACGGCTCCCTGCGGGGCTTCCTCCGCGAGAGCCGCAA | |
| | AGTGGGGCCTGGCTACCTGGGCAGTGGAGGCAGCCGCAACTCCAGCTCCCTG | |
| | GACCACCCGGATGAGCGGGCCCTCACCATGGGCGACCTCATCTCATTTGCCT | |
| | GGCAGATCTCACAGGGGATGCAGTATCTGGCCGAGATGAAGCTCGTTCATCG | |
| | GGACTTGGCAGCCAGAAACATCCTGGTAGCTGAGGGGCGGAAGATGAAGATT | |
| | TCGGATTTCGGCTTGTCCCGAGATGTTTATGAAGAGGATTCCTACGTGAAGA | |
| | GGAGCCAGGGTCGGATTCCAGTTAAATGGATGGCAATTGAATCCCTTTTTGA | |
| | TCATATCTACACCACGCAAAGTGATGTATGGTCTTTTGGTGTCCTGCTGTGG | |
| | GAGATCGTGACCCTAGGGGGAAACCCCTATCCTGGGATTCCTCCTGAGCGGC | |
| | TCTTCAACCTTCTGAAGACCGGCCACCGGATGGAGAGGCCAGACAACTGCAG | |
| | CGAGGAGATGTACCGCCTGATGCTGCAATGCTGGAAGCAGGAGCCGGACAAA | |
| | AGGCCGGTGTTTGCGGACATCAGCAAAGACCTGGAGAAGATGATGGTTAAGA | |
| | GGAGAGACTACTTGGACCTTGCGGCGTCCACTCCATCTGACTCCCTGATTTA | |
| | TGACGACGGCCTCTCAGAGGAGGAGACACCGCTGGTGGACTGTAATAATGCC | |
| | CCCCTCCCTCGAGCCCTCCCTTCCACATGGATTGAAAACAAACTCTATGGTA | |
| | GAATTTCCCATGCATTTACTAGATTCTAG | |
| SATB1- | ATGGATCATTTGAACGAGGCAACTCAGGGGAAAGAACATTCAGAAATGTCTA | 29 |
| RET | ACAATGTGAGTGATCCGAAGGGTCCACCAGCCAAGATTGCCCGCCTGGAGCA | |
| | GAACGGGAGCCCGCTAGGAAGAGGAAGGCTTGGGAGTACAGGTGCAAAAATG | |
| | CAGGGAGTGCCTTTAAAACACTCGGGCCATCTGATGAAAACCAACCTTAGGA | |
| | AAGGAACCATGCTGCCAGTTTTCTGTGTGGTGGAACATTATGAAAACGCCAT | |
| | TGAATATGATTGCAAGGAGGAGCATGCAGAATTTGTGCTGGTGAGAAAGGAT | |
| | ATGCTTTTCAACCAGCTGATCGAAATGGCATTGCTGTCTCTAGGTTATTCAC | |
| | ATAGCTCTGCTGCCCAGGCCAAAGGGCTAATCCAGGTTGGAAAGTGGAATCC | |
| | AGTTCCACTGTCTTACGTGACAGATGCCCCTGATGCTACAGTAGCAGATATG | |
| | CTTCAAGATGTGTATCATGTGGTCACATTGAAAATTCAGTTACACAGTTGCC | |
| | CCAAACTAGAAGACTTGCCTCCCGAACAATGGTCGCACACCACAGTGAGGAA | |
| | TGCTCTGAAGGACTTACTGAAAGATATGAATCAGAGTTCATTGGCCAAGGAG | |
| | TGCCCCCTTTCACAGAGTATGATTTCTTCCATTGTGAACAGTACTTACTATG | |
| | CAAATGTCTCAGCAGCAAAATGTCAAGAATTTGGAAGGTGGTACAAACATTT | |
| | CAAGAAGACAAAAGATATGATGGTTGAAATGGATAGTCTTTCTGAGCTATCC | |
| | CAGCAAGGCGCCAATCATGTCAATTTTGGCCAGCAACCAGTTCCAGGGAACA | |
| | CAGCCGAGCAGCCTCCATCCCCTGCGCAGCTCTCCCATGGCAGCCAGCCCTC | |
| | TGTCCGGACACCTCTTCCAAACCTGCACCCTGGGCTCGTATCAACACCTATC | |
| | AGTCCTCAATTGGTCAACCAGCAGCTGGTGATGGCTCAGCTGCTGAACCAGC | |
| | AGTATGCAGTGAATAGACTTTTAGCCCAGCAGTCCTTAAACCAACAATACTT | |
| | GAACCACCCTCCCCCTGTCAGTAGATCTATGAATAAGCCTTTGGAGCAACAG | |
| | GTTTCGACCAACACAGAGGTGTCTTCCGAAATCTACCAGTGGGTACGCGATG | |
| | AACTGAAACGAGCAGGAATCTCCCAGGCGGTATTTGCACGTGTGGCTTTTAA | |
| | CAGAACTCAGGAGGATCCAAAGTGGGAATTCCCTCGGAAGAACTTGGTTCTT | |
| | GGAAAAACTCTAGGAGAAGGCGAATTTGGAAAAGTGGTCAAGGCAACGGCCT | |
| | TCCATCTGAAAGGCAGAGCAGGGTACACCACGGTGGCCGTGAAGATGCTGAA | |
| | AGAGAACGCCTCCCCGAGTGAGCTGCGAGACCTGCTGTCAGAGTTCAACGTC | |
| | CTGAAGCAGGTCAACCACCCACATGTCATCAAATTGTATGGGGCCTGCAGCC | |
| | AGGATGGCCCGCTCCTCCTCATCGTGGAGTACGCCAAATACGGCTCCCTGCG | |
| | GGGCTTCCTCCGCGAGAGCCGCAAAGTGGGGCCTGGCTACCTGGGCAGTGGA | |
| | GGCAGCCGCAACTCCAGCTCCCTGGACCACCCGGATGAGCGGGCCCTCACCA | |
| | TGGGCGACCTCATCTCATTTGCCTGGCAGATCTCACAGGGGATGCAGTATCT | |
| | GGCCGAGATGAAGCTCGTTCATCGGGACTTGGCAGCCAGAAACATCCTGGTA | |
| | GCTGAGGGGCGGAAGATGAAGATTTCGGATTTCGGCTTGTCCCGAGATGTTT | |
| | ATGAAGAGGATTCCTACGTGAAGAGGAGCCAGGGTCGGATTCCAGTTAAATG | |
| | GATGGCAATTGAATCCCTTTTTGATCATATCTACACCACGCAAAGTGATGTA | |
| | TGGTCTTTTGGTGTCCTGCTGTGGGAGATCGTGACCCTAGGGGGAAACCCCT | |
| | ATCCTGGGATTCCTCCTGAGCGGCTCTTCAACCTTCTGAAGACCGGCCACCG | |
| | GATGGAGAGGCCAGACAACTGCAGCGAGGAGATGTACCGCCTGATGCTGCAA | |
| | TGCTGGAAGCAGGAGCCGGACAAAAGGCCGGTGTTTGCGGACATCAGCAAAG | |
| | ACCTGGAGAAGATGATGGTTAAGAGGAGAGACTACTTGGACCTTGCGGCGTC | |
| | CACTCCATCTGACTCCCTGATTTATGACGACGGCCTCTCAGAGGAGGAGACA | |
| | CCGCTGGTGGACTGTAATAATGCCCCCCTCCCTCGAGCCCTCCCTTCCACAT | |
| | GGATTGAAAACAAACTCTATGGTAGAATTTCCCATGCATTTACTAGATTCTA | |
| | G | |
| RET- | ATGGCGAAGGCGACGTCCGGTGCCGCGGGGCTGCGTCTGCTGTTGCTGCTGC | 30 |
| ADCY1 | TGCTGCCGCTGCTAGGCAAAGTGGCATTGGGCCTCTACTTCTCGAGGGATGC | |
| | TTACTGGGAGAAGCTGTATGTGGACCAGGCAGCCGGCACGCCCTTGCTGTAC | |
| | GTCCATGCCCTGCGGGACGCCCCTGAGGAGGTGCCCAGCTTCCGCCTGGGCC | |
| | AGCATCTCTACGGCACGTACCGCACACGGCTGCATGAGAACAACTGGATCTG | |
| | CATCCAGGAGGACACCGGCCTCCTCTACCTTAACCGGAGCCTGGACCATAGC | |
| | TCCTGGGAGAAGCTCAGTGTCCGCAACCGCGGCTTTCCCCTGCTCACCGTCT | |
| | ACCTCAAGGTCTTCCTGTCACCCACATCCCTTCGTGAGGGCGAGTGCCAGTG | |
| | GCCAGGCTGTGCCCGCGTATACTTCTCCTTCTTCAACACCTCCTTTCCAGCC | |
| | TGCAGCTCCCTCAAGCCCCGGGAGCTCTGCTTCCCAGAGACAAGGCCCTCCT | |
| | TCCGCATTCGGGAGAACCGACCCCCAGGCACCTTCCACCAGTTCCGCCTGCT | |
| | GCCTGTGCAGTTCTTGTGCCCCAACATCAGCGTGGCCTACAGGCTCCTGGAG | |
| | GGTGAGGGTCTGCCCTTCCGCTGCGCCCCGGACAGCCTGGAGGTGAGCACGC | |
| | GCTGGGCCCTGGACCGCGAGCAGCGGGAGAAGTACGAGCTGGTGGCCGTGTG | |
| | CACCGTGCACGCCGGCGCGCGCGAGGAGGTGGTGATGGTGCCCTTCCCGGTG | |
| | ACCGTGTACGACGAGGACGACTCGGCGCCCACCTTCCCCGCGGGCGTCGACA | |
| | CCGCCAGCGCCGTGGTGGAGTTCAAGCGGAAGGAGGACACCGTGGTGGCCAC | |
| | GCTGCGTGTCTTCGATGCAGACGTGGTACCTGCATCAGGGGAGCTGGTGAGG | |
| | CGGTACACAAGCACGCTGCTCCCCGGGGACACCTGGGCCCAGCAGACCTTCC | |
| | GGGTGGAACACTGGCCCAACGAGACCTCGGTCCAGGCCAACGGCAGCTTCGT | |
| | GCGGGCGACCGTACATGACTATAGGCTGGTTCTCAACCGGAACCTCTCCATC | |
| | TCGGAGAACCGCACCATGCAGCTGGCGGTGCTGGTCAATGACTCAGACTTCC | |
| | AGGGCCCAGGAGCGGGCGTCCTCTTGCTCCACTTCAACGTGTCGGTGCTGCC | |
| | GGTCAGCCTGCACCTGCCCAGTACCTACTCCCTCTCCGTGAGCAGGAGGGCT | |
| | CGCCGATTTGCCCAGATCGGGAAAGTCTGTGTGGAAAACTGCCAGGCATTCA | |
| | GTGGCATCAACGTCCAGTACAAGCTGCATTCCTCTGGTGCCAACTGCAGCAC | |
| | GCTAGGGGTGGTCACCTCAGCCGAGGACACCTCGGGGATCCTGTTTGTGAAT | |
| | GACACCAAGGCCCTGCGGCGGCCCAAGTGTGCCGAACTTCACTACATGGTGG | |
| | TGGCCACCGACCAGCAGACCTCTAGGCAGGCCCAGGCCCAGCTGCTTGTAAC | |
| | AGTGGAGGGGTCATATGTGGCCGAGGAGGCGGGCTGCCCCCTGTCCTGTGCA | |
| | GTCAGCAAGAGACGGCTGGAGTGTGAGGAGTGTGGCGGCCTGGGCTCCCCAA | |
| | CAGGCAGGTGTGAGTGGAGGCAAGGAGATGGCAAAGGGATCACCAGGAACTT | |
| | CTCCACCTGCTCTCCCAGCACCAAGACCTGCCCCGACGGCCACTGCGATGTT | |
| | GTGGAGACCCAAGACATCAACATTTGCCCTCAGGACTGCCTCCGGGGCAGCA | |
| | TTGTTGGGGGACACGAGCCTGGGGAGCCCCGGGGGATTAAAGCTGGCTATGG | |
| | CACCTGCAACTGCTTCCCTGAGGAGGAGAAGTGCTTCTGCGAGCCCGAAGAC | |
| | ATCCAGGATCCACTGTGCGACGAGCTGTGCCGCACGGTGATCGCAGCCGCTG | |
| | TCCTCTTCTCCTTCATCGTCTCGGTGCTGCTGTCTGCCTTCTGCATCCACTG | |
| | CTACCACAAGTTTGCCCACAAGCCACCCATCTCCTCAGCTGAGATGACCTTC | |
| | CGGAGGCCCGCCCAGGCCTTCCCGGTCAGCTACTCCTCTTCCGGTGCCCGCC | |
| | GGCCCTCGCTGGACTCCATGGAGAACCAGGTCTCCGTGGATGCCTTCAAGAT | |
| | CCTGTGTTTTCCAGGGTGCCTGACGATTCAGATTCGCACTGTCCTGTGTATT | |
| | TTCATAGTGGTCTTAATCTACTCAGTAGCCCAAGGTTGTGTGGTGGGCTGCC | |
| | TGCCTTGGGCCTGGAGCTCCAAGCCCAACAGTTCCCTGGTGGTCCTTTCGTC | |
| | TGGGGGCCAGCGCACAGCCCTGCCCACCCTGCCCTGCGAGTCTACACACCAT | |
| | GCCCTGCTCTGCTGCCTGGTGGGCACCCTCCCGCTAGCCATATTTTTCCGGG | |
| | TGTCCTCCTTGCCAAAAATGATCCTGCTCTCCGGGCTCACCACGTCCTACAT | |
| | CCTCGTTCTGGAGCTCAGCGGATACACCAGGACTGGGGGTGGTGCCGTCTCC | |
| | GGGCGCAGCTACGAGCCGATTGTGGCCATCCTGCTCTTCTCCTGTGCGCTGG | |
| | CCCTGCATGCCAGGCAGGTGGACATCAGGCTGAGGCTGGACTACCTCTGGGC | |
| | CGCACAGGCAGAGGAGGAGCGAGAGGACATGGAGAAGGTGAAGCTGGACAAC | |
| | AGGCGCATCCTCTTCAACCTCCTGCCGGCCCACGTCGCCCAGCACTTCCTCA | |
| | TGTCCAACCCTCGGAACATGGACCTCTACTACCAGTCCTACTCCCAGGTGGG | |
| | CGTCATGTTTGCCTCCATCCCCAACTTCAATGACTTCTACATCGAGCTGGAC | |
| | GGCAACAACATGGGGGTGGAGTGTCTGCGGCTTCTCAACGAGATCATCGCCG | |
| | ACTTTGACGAGCTCATGGAAAAAGACTTTTACAAGGACATAGAGAAGATCAA | |
| | GACCATCGGGAGCACCTACATGGCCGCTGTGGGGCTAGCGCCCACCTCGGGG | |
| | ACCAAGGCTAAGAAGTCCATCTCCTCCCACCTGAGCACGCTGGCGGACTTTG | |
| | CCATTGAGATGTTTGACGTTCTGGATGAAATCAACTACCAGTCTTACAACGA | |
| | CTTTGTCCTCCGAGTTGGCATCAATGTTGGCCCTGTGGTGGCTGGAGTGATT | |
| | GGCGCTCGCAGGCCCCAGTACGACATCTGGGGAAACACAGTCAACGTGGCCA | |
| | GTCGGATGGATAGCACAGGGGTCCAGGGCAGAATCCAGGTGACTGAGGAAGT | |
| | CCACCGGCTGCTGAGAAGGTGCCCCTACCACTTTGTGTGCCGAGGCAAAGTC | |
| | AGTGTCAAGGGCAAAGGCGAGATGTTGACATACTTTCTAGAAGGCAGGACTG | |
| | ATGGAAACGGCTCCCAAATCAGGTCCCTGGGCTTGGATCGGAAAATGTGTCC | |
| | ATTTGGGAGAGCTGGCCTTCAGGGCAGACGTCCCCCCGTGTGCCCCATGCCT | |
| | GGCGTCTCAGTCAGGGCTGGGCTCCCTCCACACTCCCCAGGCCAGTACCTGC | |
| | CCTCTGCAGCAGCTGGGAAGGAGGCTTAG | |
| RET- | ATGGCGAAGGCGACGTCCGGTGCCGCGGGGCTGCGTCTGCTGTTGCTGCTGC | 31 |
| ZNF248 | TGCTGCCGCTGCTAGGCAAAGTGGCATTGGGCCTCTACTTCTCGAGGGATGC | |
| | TTACTGGGAGAAGCTGTATGTGGACCAGGCAGCCGGCACGCCCTTGCTGTAC | |
| | GTCCATGCCCTGCGGGACGCCCCTGAGGAGGTGCCCAGCTTCCGCCTGGGCC | |
| | AGCATCTCTACGGCACGTACCGCACACGGCTGCATGAGAACAACTGGATCTG | |
| | CATCCAGGAGGACACCGGCCTCCTCTACCTTAACCGGAGCCTGGACCATAGC | |
| | TCCTGGGAGAAGCTCAGTGTCCGCAACCGCGGCTTTCCCCTGCTCACCGTCT | |
| | ACCTCAAGGTCTTCCTGTCACCCACATCCCTTCGTGAGGGCGAGTGCCAGTG | |
| | GCCAGGCTGTGCCCGCGTATACTTCTCCTTCTTCAACACCTCCTTTCCAGCC | |
| | TGCAGCTCCCTCAAGCCCCGGGAGCTCTGCTTCCCAGAGACAAGGCCCTCCT | |
| | TCCGCATTCGGGAGAACCGACCCCCAGGCACCTTCCACCAGTTCCGCCTGCT | |
| | GCCTGTGCAGTTCTTGTGCCCCAACATCAGCGTGGCCTACAGGCTCCTGGAG | |
| | GGTGAGGGTCTGCCCTTCCGCTGCGCCCCGGACAGCCTGGAGGTGAGCACGC | |
| | GCTGGGCCCTGGACCGCGAGCAGCGGGAGAAGTACGAGCTGGTGGCCGTGTG | |
| | CACCGTGCACGCCGGCGCGCGCGAGGAGGTGGTGATGGTGCCCTTCCCGGTG | |
| | ACCGTGTACGACGAGGACGACTCGGCGCCCACCTTCCCCGCGGGCGTCGACA | |
| | CCGCCAGCGCCGTGGTGGAGTTCAAGCGGAAGGAGGACACCGTGGTGGCCAC | |
| | GCTGCGTGTCTTCGATGCAGACGTGGTACCTGCATCAGGGGAGCTGGTGAGG | |
| | CGGTACACAAGCACGCTGCTCCCCGGGGACACCTGGGCCCAGCAGACCTTCC | |
| | GGGTGGAACACTGGCCCAACGAGACCTCGGTCCAGGCCAACGGCAGCTTCGT | |
| | GCGGGCGACCGTACATGACTATAGGCTGGTTCTCAACCGGAACCTCTCCATC | |
| | TCGGAGAACCGCACCATGCAGCTGGCGGTGCTGGTCAATGACTCAGACTTCC | |
| | AGGGCCCAGGAGCGGGCGTCCTCTTGCTCCACTTCAACGTGTCGGTGCTGCC | |
| | GGTCAGCCTGCACCTGCCCAGTACCTACTCCCTCTCCGTGAGCAGGAGGGCT | |
| | CGCCGATTTGCCCAGATCGGGAAAGTCTGTGTGGAAAACTGCCAGGCATTCA | |
| | GTGGCATCAACGTCCAGTACAAGCTGCATTCCTCTGGTGCCAACTGCAGCAC | |
| | GCTAGGGGTGGTCACCTCAGCCGAGGACACCTCGGGGATCCTGTTTGTGAAT | |
| | GACACCAAGGCCCTGCGGCGGCCCAAGTGTGCCGAACTTCACTACATGGTGG | |
| | TGGCCACCGACCAGCAGACCTCTAGGCAGGCCCAGGCCCAGCTGCTTGTAAC | |
| | AGTGGAGGGGTCATATGTGGCCGAGGAGGCGGGCTGCCCCCTGTCCTGTGCA | |
| | GTCAGCAAGAGACGGCTGGAGTGTGAGGAGTGTGGCGGCCTGGGCTCCCCAA | |
| | CAGGCAGGTGTGAGTGGAGGCAAGGAGATGGCAAAGGGATCACCAGGAACTT | |
| | CTCCACCTGCTCTCCCAGCACCAAGACCTGCCCCGACGGCCACTGCGATGTT | |
| | GTGGAGACCCAAGACATCAACATTTGCCCTCAGGACTGCCTCCGGGGCAGCA | |
| | TTGTTGGGGGACACGAGCCTGGGGAGCCCCGGGGGATTAAAGCTGGCTATGG | |
| | CACCTGCAACTGCTTCCCTGAGGAGGAGAAGTGCTTCTGCGAGCCCGAAGAC | |
| | ATCCAGGATCCACTGTGCGACGAGCTGTGCCGCACGGTGATCGCAGCCGCTG | |
| | TCCTCTTCTCCTTCATCGTCTCGGTGCTGCTGTCTGCCTTCTGCATCCACTG | |
| | CTACCACAAGTTTGCCCACAAGCCACCCATCTCCTCAGCTGAGATGACCTTC | |
| | CGGAGGCCCGCCCAGGCCTTCCCGGTCAGCTACTCCTCTTCCGGTGCCCGCC | |
| | GGCCCTCGCTGGACTCCATGGAGAACCAGGTCTCCGTGGATGCCTTCAAGAT | |
| | CCTGGAACAAGTGTCATTCAAGGATGTATGTGTGGACTTCACTCAGGAAGAG | |
| | TGGTATCTGCTGGACCCTGCTCAGAAGATTCTATACAGAGATGTGATCCTGG | |
| | AAAATTATAGCAATCTTGTCTCAGTAGGGTATTGCATTACTAAACCAGAAGT | |
| | GATCTTTAAGATCGAGCAAGGAGAAGAGCCCTGGATATTAGAAAAAGGATTC | |
| | CCAAGCCAGTGCCACCCAGAAAGGAAATGGAAAGTTGATGACGTGTTAGAGA | |
| | GCAGCCAGGAAAATGAAGATGACCATTTTTGGGAGCTTCTATTCCACAACAA | |
| | CAAAACAGTAAGTGTAGAAAATGGAGATAGAGGAAGCAAAACTTTCAATTTG | |
| | GGCACAGACCCTGTTTCTTTAAGAAATTATCCCTATAAAATATGTGACTCAT | |
| | GTGAAATGAATTTGAAAAATATTTCGGGCTTAATTATTAGTAAAAAGAACTG | |
| | TTCCAGAAAGAAGCCTGATGAGTTTAATGTATGTGAGAAATTGCTCCTTGAT | |
| | ATTAGGCATGAGAAAATCCCTATTGGAGAGAAGTCTTATAAATATGATCAAA | |
| | AAAGGAATGCCATTAATTATCACCAGGATCTCAGTCAGCCAAGTTTTGGCCA | |
| | ATCTTTTGAGTATAGTAAAAATGGACAAGGCTTCCATGATGAGGCAGCATTT | |
| | TTTACAAATAAGAGATCTCAGATAGGAGAGACAGTCTGTAAATATAACGAAT | |
| | GTGGAAGAACCTTCATTGAAAGTTTAAAGCTGAATATATCTCAAAGACCTCA | |
| | TTTGGAAATGGAGCCGTATGGATGCAGTATTTGCGGGAAGTCCTTCTGCATG | |
| | AATTTAAGGTTTGGACATCAGAGAGCTCTTACAAAGGACAATCCTTATGAAT | |
| | ATAATGAATATGGGGAAATCTTCTGTGACAATTCAGCTTTCATTATCCATCA | |
| | GGGAGCTTACACAAGAAAGATTCTCCGTGAATATAAAGTGAGTGACAAAACC | |
| | TGGGAAAAGTCAGCTCTCTTAAAACATCAAATAGTACACATGGGGGGAAAGT | |
| | CTTATGATTACAATGAAAATGGGAGTAATTTCAGCAAGAAGTCACATCTTAC | |
| | CCAGCTTCGGAGAGCTCACACAGGAGAAAAAACCTTTGAATGTGGTGAATGT | |
| | GGGAAAACCTTCTGGGAGAAGTCAAACCTCACTCAACATCAGAGAACACACA | |
| | CAGGAGAGAAGCCCTATGAATGTACTGAATGTGGGAAAGCCTTTTGCCAGAA | |
| | ACCACACCTGACCAACCATCAGCGAACACATACAGGAGAAAAACCCTATGAA | |
| | TGTAAGCAATGTGGAAAAACATTCTGTGTGAAGTCAAACCTCACTGAACATC | |
| | AGAGAACACACACAGGGGAGAAGCCCTATGAATGTAATGCATGTGGGAAATC | |
| | CTTCTGCCACAGATCAGCCCTCACTGTGCATCAGAGAACACACACAGGGGAG | |
| | AAACCGTTTATATGTAATGAATGTGGAAAATCCTTCTGTGTGAAGTCAAACC | |
| | TCATTGTACATCAAAGAACTCACACTGGGGAGAAACCATATAAGTGTAATGA | |
| | ATGTGGGAAAACCTTCTGTGAAAAATCAGCTCTCACTAAACATCAGAGGACT | |
| | CACACAGGGGAGAAGCCGTATGAGTGTAATGCATGTGGGAAGACCTTTAGTC | |
| | AGAGGTCAGTGCTCACCAAACATCAGAGAATTCACACAAGGGTGAAAGCTCT | |
| | TTCAACATCCTGA | |
| RET- | ATGGCGAAGGCGACGTCCGGTGCCGCGGGGCTGCGTCTGCTGTTGCTGCTGC | 32 |
| AGBL4 | TGCTGCCGCTGCTAGGCAAAGTGGCATTGGGCCTCTACTTCTCGAGGGATGC | |
| | TTACTGGGAGAAGCTGTATGTGGACCAGGCAGCCGGCACGCCCTTGCTGTAC | |
| | GTCCATGCCCTGCGGGACGCCCCTGAGGAGGTGCCCAGCTTCCGCCTGGGCC | |
| | AGCATCTCTACGGCACGTACCGCACACGGCTGCATGAGAACAACTGGATCTG | |
| | CATCCAGGAGGACACCGGCCTCCTCTACCTTAACCGGAGCCTGGACCATAGC | |
| | TCCTGGGAGAAGCTCAGTGTCCGCAACCGCGGCTTTCCCCTGCTCACCGTCT | |
| | ACCTCAAGGTCTTCCTGTCACCCACATCCCTTCGTGAGGGCGAGTGCCAGTG | |
| | GCCAGGCTGTGCCCGCGTATACTTCTCCTTCTTCAACACCTCCTTTCCAGCC | |
| | TGCAGCTCCCTCAAGCCCCGGGAGCTCTGCTTCCCAGAGACAAGGCCCTCCT | |
| | TCCGCATTCGGGAGAACCGACCCCCAGGCACCTTCCACCAGTTCCGCCTGCT | |
| | GCCTGTGCAGTTCTTGTGCCCCAACATCAGCGTGGCCTACAGGCTCCTGGAG | |
| | GGTGAGGGTCTGCCCTTCCGCTGCGCCCCGGACAGCCTGGAGGTGAGCACGC | |
| | GCTGGGCCCTGGACCGCGAGCAGCGGGAGAAGTACGAGCTGGTGGCCGTGTG | |
| | CACCGTGCACGCCGGCGCGCGCGAGGAGGTGGTGATGGTGCCCTTCCCGGTG | |
| | ACCGTGTACGACGAGGACGACTCGGCGCCCACCTTCCCCGCGGGCGTCGACA | |
| | CCGCCAGCGCCGTGGTGGAGTTCAAGCGGAAGGAGGACACCGTGGTGGCCAC | |
| | GCTGCGTGTCTTCGATGCAGACGTGGTACCTGCATCAGGGGAGCTGGTGAGG | |
| | CGGTACACAAGCACGCTGCTCCCCGGGGACACCTGGGCCCAGCAGACCTTCC | |
| | GGGTGGAACACTGGCCCAACGAGACCTCGGTCCAGGCCAACGGCAGCTTCGT | |
| | GCGGGCGACCGTACATGACTATAGGCTGGTTCTCAACCGGAACCTCTCCATC | |
| | TCGGAGAACCGCACCATGCAGCTGGCGGTGCTGGTCAATGACTCAGACTTCC | |
| | AGGGCCCAGGAGCGGGCGTCCTCTTGCTCCACTTCAACGTGTCGGTGCTGCC | |
| | GGTCAGCCTGCACCTGCCCAGTACCTACTCCCTCTCCGTGAGCAGGAGGGCT | |
| | CGCCGATTTGCCCAGATCGGGAAAGTCTGTGTGGAAAACTGCCAGGCATTCA | |
| | GTGGCATCAACGTCCAGTACAAGCTGCATTCCTCTGGTGCCAACTGCAGCAC | |
| | GCTAGGGGTGGTCACCTCAGCCGAGGACACCTCGGGGATCCTGTTTGTGAAT | |
| | GACACCAAGGCCCTGCGGCGGCCCAAGTGTGCCGAACTTCACTACATGGTGG | |
| | TGGCCACCGACCAGCAGACCTCTAGGCAGGCCCAGGCCCAGCTGCTTGTAAC | |
| | AGTGGAGGGGTCATATGTGGCCGAGGAGGCGGGCTGCCCCCTGTCCTGTGCA | |
| | GTCAGCAAGAGACGGCTGGAGTGTGAGGAGTGTGGCGGCCTGGGCTCCCCAA | |
| | CAGGCAGGTGTGAGTGGAGGCAAGGAGATGGCAAAGGGATCACCAGGAACTT | |
| | CTCCACCTGCTCTCCCAGCACCAAGACCTGCCCCGACGGCCACTGCGATGTT | |
| | GTGGAGACCCAAGACATCAACATTTGCCCTCAGGACTGCCTCCGGGGCAGCA | |
| | TTGTTGGGGGACACGAGCCTGGGGAGCCCCGGGGGATTAAAGCTGGCTATGG | |
| | CACCTGCAACTGCTTCCCTGAGGAGGAGAAGTGCTTCTGCGAGCCCGAAGAC | |
| | ATCCAGGATCCACTGTGCGACGAGCTGTGCCGCACGGTGATCGCAGCCGCTG | |
| | TCCTCTTCTCCTTCATCGTCTCGGTGCTGCTGTCTGCCTTCTGCATCCACTG | |
| | CTACCACAAGTTTGCCCACAAGCCACCCATCTCCTCAGCTGAGATGACCTTC | |
| | CGGAGGCCCGCCCAGGCCTTCCCGGTCAGCTACTCCTCTTCCGGTGCCCGCC | |
| | GGCCCTCGCTGGACTCCATGGAGAACCAGGTCTCCGTGGATGCCTTCAAGAT | |
| | CCTGGAGGATCCAAAGTGGGAATTCCCTCGGAAGAACTTGGTTCTTGGAAAA | |
| | ACTCTAGGAGAAGGCGAATTTGGAAAAGTGGTCAAGGCAACGGCCTTCCATC | |
| | TGAAAGGCAGAGCAGGGTACACCACGGTGGCCGTGAAGATGCTGAAAGGTAA | |
| | CCTGGGCCGGGTGGACCAGGTCTCTGAGTTTGAGTATGATCTGTTCATTAGG | |
| | CCGGACACCTGTAATCCACGCTTCCGAGTCTGGTTCAACTTTACTGTTGAAA | |
| | ATGTGAAAGAATCACAGAGGGTCATTTTCAACATTGTTAACTTCAGTAAAAC | |
| | CAAGAGTCTCTATAGAGATGGGATGGCCCCTATGGTGAAATCTACCAGCAGA | |
| | CCAAAATGGCAAAGGCTGCCACCCAAAAATGTTTACTACTACCGCTGCCCGG | |
| | ACCATAGGAAGAACTATGTGATGTCCTTTGCCTTTTGTTTTGACCGAGAAGA | |
| | AGATATTTACCAGTTTGCTTACTGCTACCCATATACATACACTCGCTTCCAA | |
| | CATTACCTTGACAGCCTGCAAAAGAGAAACATGGATTACTTCTTTCGGGAGC | |
| | AGCTGGGCCAGAGTGTGCAACAACGAAAGCTTGACCTCCTGACGATAACCAG | |
| | CCCTGACAATCTCCGGGAAGGGGCAGAGCAGAAGGTGGTATTCATCACAGGA | |
| | CGAGTCCACCCAGGGGAAACACCCTCATCATTTGTGTGCCAAGGGATCATTG | |
| | ACTTCCTTGTAAGCCAGCACCCTATTGCCTGTGTCCTCCGGGAATACCTGGT | |
| | CTTCAAGATCGCACCAATGCTCAATCCTGATGGAGTCTACCTGGGCAATTAC | |
| | AGGTGTTCTCTGATGGGATTTGATCTGAATCGTCACTGGCTGGATCCCTCTC | |
| | CATGGGTCCATCCTACCCTGCATGGAGTGAAACAACTCATCGTCCAGATGTA | |
| | CAACGACCCAAAAACAAGCCTGGAGTTTTATATTGACATCCATGCCCACTCC | |
| | ACCATGATGAATGGCTTCATGTATGGCAACATCTTTGAGGATGAGGAACGGT | |
| | TCCAGAGGCAGGCCATTTTTCCCAAGCTCCTCTGCCAGAATGCTGAGGACTT | |
| | CTCCTATTCCAGCACATCCTTTAACCGGGACGCTGTGAAAGCAGGAACTGGC | |
| | CGTCGCTTCCTCGGTGGACTCCTGGACCACACTTCCTATTGCTACACCCTAG | |
| | AGGTCTCCTTCTACAGCTACATCATCAGTGGCACCACGGCTGCTGTGCCCTA | |
| | CACTGAAGAAGCCTATATGAAGCTGGGGCGGAATGTGGCAAGAACCTTTTTG | |
| | GACTATTATCGGCTGAACCCCGTGGTTGAAAAGGTGGCAATTCCCATGCCGA | |
| | GACTGCGAAATAAAGAAATAGAAGTCCAGAGAAGGAAAGAAAAATCCCCTCC | |
| | TTACAAGCACCCACTCCTGCGGGGCCCAGCCAGCAACTACCCCAACAGCAAA | |
| | GGGGACAAGAAGAGCTCAGTGAACCACAAAGACCCTTCAACCCCTTTTTAA | |
| RET- | ATGGCGAAGGCGACGTCCGGTGCCGCGGGGCTGCGTCTGCTGTTGCTGCTGC | 33 |
| LRMDA | TGCTGCCGCTGCTAGGCAAAGTGGCATTGGGCCTCTACTTCTCGAGGGATGC | |
| | TTACTGGGAGAAGCTGTATGTGGACCAGGCAGCCGGCACGCCCTTGCTGTAC | |
| | GTCCATGCCCTGCGGGACGCCCCTGAGGAGGTGCCCAGCTTCCGCCTGGGCC | |
| | AGCATCTCTACGGCACGTACCGCACACGGCTGCATGAGAACAACTGGATCTG | |
| | CATCCAGGAGGACACCGGCCTCCTCTACCTTAACCGGAGCCTGGACCATAGC | |
| | TCCTGGGAGAAGCTCAGTGTCCGCAACCGCGGCTTTCCCCTGCTCACCGTCT | |
| | ACCTCAAGGTCTTCCTGTCACCCACATCCCTTCGTGAGGGCGAGTGCCAGTG | |
| | GCCAGGCTGTGCCCGCGTATACTTCTCCTTCTTCAACACCTCCTTTCCAGCC | |
| | TGCAGCTCCCTCAAGCCCCGGGAGCTCTGCTTCCCAGAGACAAGGCCCTCCT | |
| | TCCGCATTCGGGAGAACCGACCCCCAGGCACCTTCCACCAGTTCCGCCTGCT | |
| | GCCTGTGCAGTTCTTGTGCCCCAACATCAGCGTGGCCTACAGGCTCCTGGAG | |
| | GGTGAGGGTCTGCCCTTCCGCTGCGCCCCGGACAGCCTGGAGGTGAGCACGC | |
| | GCTGGGCCCTGGACCGCGAGCAGCGGGAGAAGTACGAGCTGGTGGCCGTGTG | |
| | CACCGTGCACGCCGGCGCGCGCGAGGAGGTGGTGATGGTGCCCTTCCCGGTG | |
| | ACCGTGTACGACGAGGACGACTCGGCGCCCACCTTCCCCGCGGGCGTCGACA | |
| | CCGCCAGCGCCGTGGTGGAGTTCAAGCGGAAGGAGGACACCGTGGTGGCCAC | |
| | GCTGCGTGTCTTCGATGCAGACGTGGTACCTGCATCAGGGGAGCTGGTGAGG | |
| | CGGTACACAAGCACGCTGCTCCCCGGGGACACCTGGGCCCAGCAGACCTTCC | |
| | GGGTGGAACACTGGCCCAACGAGACCTCGGTCCAGGCCAACGGCAGCTTCGT | |
| | GCGGGCGACCGTACATGACTATAGGCTGGTTCTCAACCGGAACCTCTCCATC | |
| | TCGGAGAACCGCACCATGCAGCTGGCGGTGCTGGTCAATGACTCAGACTTCC | |
| | AGGGCCCAGGAGCGGGCGTCCTCTTGCTCCACTTCAACGTGTCGGTGCTGCC | |
| | GGTCAGCCTGCACCTGCCCAGTACCTACTCCCTCTCCGTGAGCAGGAGGGCT | |
| | CGCCGATTTGCCCAGATCGGGAAAGTCTGTGTGGAAAACTGCCAGGCATTCA | |
| | GTGGCATCAACGTCCAGTACAAGCTGCATTCCTCTGGTGCCAACTGCAGCAC | |
| | GCTAGGGGTGGTCACCTCAGCCGAGGACACCTCGGGGATCCTGTTTGTGAAT | |
| | GACACCAAGGCCCTGCGGCGGCCCAAGTGTGCCGAACTTCACTACATGGTGG | |
| | TGGCCACCGACCAGCAGACCTCTAGGCAGGCCCAGGCCCAGCTGCTTGTAAC | |
| | AGTGGAGGGGTCATATGTGGCCGAGGAGGCGGGCTGCCCCCTGTCCTGTGCA | |
| | GTCAGCAAGAGACGGCTGGAGTGTGAGGAGTGTGGCGGCCTGGGCTCCCCAA | |
| | CAGGCAGGTGTGAGTGGAGGCAAGGAGATGGCAAAGGGATCACCAGGAACTT | |
| | CTCCACCTGCTCTCCCAGCACCAAGACCTGCCCCGACGGCCACTGCGATGTT | |
| | GTGGAGACCCAAGACATCAACATTTGCCCTCAGGACTGCCTCCGGGGCAGCA | |
| | TTGTTGGGGGACACGAGCCTGGGGAGCCCCGGGGGATTAAAGCTGGCTATGG | |
| | CACCTGCAACTGCTTCCCTGAGGAGGAGAAGTGCTTCTGCGAGCCCGAAGAC | |
| | ATCCAGGGTGTCCTGGGGAAGTGTCGCTACGTTTACTATGGGAAAAACTCAG | |
| | AGGGCAACAGGTTTATCCGAGATGACCAGCTCTGA | |
| RET- | ATGGCGAAGGCGACGTCCGGTGCCGCGGGGCTGCGTCTGCTGTTGCTGCTGC | 34 |
| ARHGAP19 | TGCTGCCGCTGCTAGGCAAAGTGGCATTGGGCCTCTACTTCTCGAGGGATGC | |
| | TTACTGGGAGAAGCTGTATGTGGACCAGGCAGCCGGCACGCCCTTGCTGTAC | |
| | GTCCATGCCCTGCGGGACGCCCCTGAGGAGGTGCCCAGCTTCCGCCTGGGCC | |
| | AGCATCTCTACGGCACGTACCGCACACGGCTGCATGAGAACAACTGGATCTG | |
| | CATCCAGGAGGACACCGGCCTCCTCTACCTTAACCGGAGCCTGGACCATAGC | |
| | TCCTGGGAGAAGCTCAGTGTCCGCAACCGCGGCTTTCCCCTGCTCACCGTCT | |
| | ACCTCAAGGTCTTCCTGTCACCCACATCCCTTCGTGAGGGCGAGTGCCAGTG | |
| | GCCAGGCTGTGCCCGCGTATACTTCTCCTTCTTCAACACCTCCTTTCCAGCC | |
| | TGCAGCTCCCTCAAGCCCCGGGAGCTCTGCTTCCCAGAGACAAGGCCCTCCT | |
| | TCCGCATTCGGGAGAACCGACCCCCAGGCACCTTCCACCAGTTCCGCCTGCT | |
| | GCCTGTGCAGTTCTTGTGCCCCAACATCAGCGTGGCCTACAGGCTCCTGGAG | |
| | GGTGAGGGTCTGCCCTTCCGCTGCGCCCCGGACAGCCTGGAGGTGAGCACGC | |
| | GCTGGGCCCTGGACCGCGAGCAGCGGGAGAAGTACGAGCTGGTGGCCGTGTG | |
| | CACCGTGCACGCCGGCGCGCGCGAGGAGGTGGTGATGGTGCCCTTCCCGGTG | |
| | ACCGTGTACGACGAGGACGACTCGGCGCCCACCTTCCCCGCGGGCGTCGACA | |
| | CCGCCAGCGCCGTGGTGGAGTTCAAGCGGAAGGAGGACACCGTGGTGGCCAC | |
| | GCTGCGTGTCTTCGATGCAGACGTGGTACCTGCATCAGGGGAGCTGGTGAGG | |
| | CGGTACACAAGCACGCTGCTCCCCGGGGACACCTGGGCCCAGCAGACCTTCC | |
| | GGGTGGAACACTGGCCCAACGAGACCTCGGTCCAGGCCAACGGCAGCTTCGT | |
| | GCGGGCGACCGTACATGACTATAGGCTGGTTCTCAACCGGAACCTCTCCATC | |
| | TCGGAGAACCGCACCATGCAGCTGGCGGTGCTGGTCAATGACTCAGACTTCC | |
| | AGGGCCCAGGAGCGGGCGTCCTCTTGCTCCACTTCAACGTGTCGGTGCTGCC | |
| | GGTCAGCCTGCACCTGCCCAGTACCTACTCCCTCTCCGTGAGCAGGAGGGCT | |
| | CGCCGATTTGCCCAGATCGGGAAAGTCTGTGTGGAAAACTGCCAGGCATTCA | |
| | GTGGCATCAACGTCCAGTACAAGCTGCATTCCTCTGGTGCCAACTGCAGCAC | |
| | GCTAGGGGTGGTCACCTCAGCCGAGGACACCTCGGGGATCCTGTTTGTGAAT | |
| | GACACCAAGGCCCTGCGGCGGCCCAAGTGTGCCGAACTTCACTACATGGTGG | |
| | TGGCCACCGACCAGCAGACCTCTAGGCAGGCCCAGGCCCAGCTGCTTGTAAC | |
| | AGTGGAGGGGTCATATGTGGCCGAGGAGGCGGGCTGCCCCCTGTCCTGTGCA | |
| | GTCAGCAAGAGACGGCTGGAGTGTGAGGAGTGTGGCGGCCTGGGCTCCCCAA | |
| | CAGGCAGGTGTGAGTGGAGGCAAGGAGATGGCAAAGGGATCACCAGGAACTT | |
| | CTCCACCTGCTCTCCCAGCACCAAGACCTGCCCCGACGGCCACTGCGATGTT | |
| | GTGGAGACCCAAGACATCAACATTTGCCCTCAGGACTGCCTCCGGGGCAGCA | |
| | TTGTTGGGGGACACGAGCCTGGGGAGCCCCGGGGGATTAAAGCTGGCTATGG | |
| | CACCTGCAACTGCTTCCCTGAGGAGGAGAAGTGCTTCTGCGAGCCCGAAGAC | |
| | ATCCAGGATCCACTGTGCGACGAGCTGTGCCGCACGGTGATCGCAGCCGCTG | |
| | TCCTCTTCTCCTTCATCGTCTCGGTGCTGCTGTCTGCCTTCTGCATCCACTG | |
| | CTACCACAAGTTTGCCCACAAGCCACCCATCTCCTCAGCTGAGATGACCTTC | |
| | CGGAGGCCCGCCCAGGCCTTCCCGGTCAGCTACTCCTCTTCCGGTGCCCGCC | |
| | GGCCCTCGCTGGACTCCATGGAGAACCAGGTCTCCGTGGATGCCTTCAAGAT | |
| | CCTGGCTCCTGCTTACATTCGGGAGTGTGCGAGATTGCACTATTTGGGATCC | |
| | AGAACTCAGGCATCAAAGGATGACCTTGACCTCATAGCTTCATGTCATACTA | |
| | AGTCCTTTCAGCTGGCAAAGTCTCAGAAACGGAACCGGGTAGATTCCTGCCC | |
| | TCACCAGGAGGAGACCCAGCACCATACGGAAGAGGCACTGAGAGAGCTGTTT | |
| | CAACACGTTCATGATATGCCAGAGTCAGCAAAGAAGAAACAACTTATTAGAC | |
| | AGTTTAATAAGCAATCATTGACCCAGACACCAGGGCGAGAACCTTCTACTTC | |
| | CCAGGTACAAAAGAGGGCTCGTTCGCGCTCCTTCAGTGGGCTTATTAAGCGG | |
| | AAGGTCCTGGGAAATCAGATGATGTCAGAAAAGAAAAAGAAGAACCCTACTC | |
| | CAGAATCTGTGGCCATTGGTGAATTGAAGGGAACCAGCAAAGAAAATAGGAA | |
| | CTTATTATTTTCTGGCTCTCCAGCTGTCACGATGACACCAACAAGATTGAAG | |
| | TGGTCTGAAGGGAAGAAAGAGGGGAAAAAAGGATTTCTCTGA | |
| RET- | ATGGCGAAGGCGACGTCCGGTGCCGCGGGGCTGCGTCTGCTGTTGCTGCTGC | 35 |
| CPEB3 | TGCTGCCGCTGCTAGGCAAAGTGGCATTGGGCCTCTACTTCTCGAGGGATGC | |
| | TTACTGGGAGAAGCTGTATGTGGACCAGGCAGCCGGCACGCCCTTGCTGTAC | |
| | GTCCATGCCCTGCGGGACGCCCCTGAGGAGGTGCCCAGCTTCCGCCTGGGCC | |
| | AGCATCTCTACGGCACGTACCGCACACGGCTGCATGAGAACAACTGGATCTG | |
| | CATCCAGGAGGACACCGGCCTCCTCTACCTTAACCGGAGCCTGGACCATAGC | |
| | TCCTGGGAGAAGCTCAGTGTCCGCAACCGCGGCTTTCCCCTGCTCACCGTCT | |
| | ACCTCAAGGTCTTCCTGTCACCCACATCCCTTCGTGAGGGCGAGTGCCAGTG | |
| | GCCAGGCTGTGCCCGCGTATACTTCTCCTTCTTCAACACCTCCTTTCCAGCC | |
| | TGCAGCTCCCTCAAGCCCCGGGAGCTCTGCTTCCCAGAGACAAGGCCCTCCT | |
| | TCCGCATTCGGGAGAACCGACCCCCAGGCACCTTCCACCAGTTCCGCCTGCT | |
| | GCCTGTGCAGTTCTTGTGCCCCAACATCAGCGTGGCCTACAGGCTCCTGGAG | |
| | GGTGAGGGTCTGCCCTTCCGCTGCGCCCCGGACAGCCTGGAGGTGAGCACGC | |
| | GCTGGGCCCTGGACCGCGAGCAGCGGGAGAAGTACGAGCTGGTGGCCGTGTG | |
| | CACCGTGCACGCCGGCGCGCGCGAGGAGGTGGTGATGGTGCCCTTCCCGGTG | |
| | ACCGTGTACGACGAGGACGACTCGGCGCCCACCTTCCCCGCGGGCGTCGACA | |
| | CCGCCAGCGCCGTGGTGGAGTTCAAGCGGAAGGAGGACACCGTGGTGGCCAC | |
| | GCTGCGTGTCTTCGATGCAGACGTGGTACCTGCATCAGGGGAGCTGGTGAGG | |
| | CGGTACACAAGCACGCTGCTCCCCGGGGACACCTGGGCCCAGCAGACCTTCC | |
| | GGGTGGAACACTGGCCCAACGAGACCTCGGTCCAGGCCAACGGCAGCTTCGT | |
| | GCGGGCGACCGTACATGACTATAGGCTGGTTCTCAACCGGAACCTCTCCATC | |
| | TCGGAGAACCGCACCATGCAGCTGGCGGTGCTGGTCAATGACTCAGACTTCC | |
| | AGGGCCCAGGAGCGGGCGTCCTCTTGCTCCACTTCAACGTGTCGGTGCTGCC | |
| | GGTCAGCCTGCACCTGCCCAGTACCTACTCCCTCTCCGTGAGCAGGAGGGCT | |
| | CGCCGATTTGCCCAGATCGGGAAAGTCTGTGTGGAAAACTGCCAGGCATTCA | |
| | GTGGCATCAACGTCCAGTACAAGCTGCATTCCTCTGGTGCCAACTGCAGCAC | |
| | GCTAGGGGTGGTCACCTCAGCCGAGGACACCTCGGGGATCCTGTTTGTGAAT | |
| | GACACCAAGGCCCTGCGGCGGCCCAAGTGTGCCGAACTTCACTACATGGTGG | |
| | TGGCCACCGACCAGCAGACCTCTAGGCAGGCCCAGGCCCAGCTGCTTGTAAC | |
| | AGTGGAGGGGTCATATGTGGCCGAGGAGGCGGGCTGCCCCCTGTCCTGTGCA | |
| | GTCAGCAAGAGACGGCTGGAGTGTGAGGAGTGTGGCGGCCTGGGCTCCCCAA | |
| | CAGGCAGGTGTGAGTGGAGGCAAGGAGATGGCAAAGGGATCACCAGGAACTT | |
| | CTCCACCTGCTCTCCCAGCACCAAGACCTGCCCCGACGGCCACTGCGATGTT | |
| | GTGGAGACCCAAGACATCAACATTTGCCCTCAGGACTGCCTCCGGGGCAGCA | |
| | TTGTTGGGGGACACGAGCCTGGGGAGCCCCGGGGGATTAAAGCTGGCTATGG | |
| | CACCTGCAACTGCTTCCCTGAGGAGGAGAAGTGCTTCTGCGAGCCCGAAGAC | |
| | ATCCAGGATCCACTGTGCGACGAGCTGTGCCGCACGGTGATCGCAGCCGCTG | |
| | TCCTCTTCTCCTTCATCGTCTCGGTGCTGCTGTCTGCCTTCTGCATCCACTG | |
| | CTACCACAAGTTTGCCCACAAGCCACCCATCTCCTCAGCTGAGATGACCTTC | |
| | CGGAGGCCCGCCCAGGCCTTCCCGGTCAGCTACTCCTCTTCCGGTGCCCGCC | |
| | GGCCCTCGCTGGACTCCATGGAGAACCAGGTCTCCGTGGATGCCTTCAAGAT | |
| | CCTGGTGCAAATTCGACCATGGAACCTAAGTGACAGTGACTTTGTAATGGAT | |
| | GGTTCTCAGCCTTTGGACCCCAGAAAAACTATCTTTGTTGGGGGAGTTCCAC | |
| | GACCCCTTCGAGCTGTTGAACTGGCAATGATCATGGACCGTTTGTACGGTGG | |
| | TGTCTGCTATGCTGGCATTGATACGGACCCAGAGCTGAAGTACCCCAAAGGT | |
| | GCTGGCCGCGTGGCATTCTCCAATCAGCAGAGTTACATTGCAGCCATCAGCG | |
| | CTCGTTTTGTGCAGCTTCAGCACAATGACATTGACAAACGGGTTGAAGTAAA | |
| | GCCATATGTGCTGGATGATCAGATGTGTGATGAGTGCCAGGGCACACGCTGT | |
| | GGTGGGAAGTTTGCCCCGTTCTTCTGTGCCAACGTCACCTGTCTGCAGTATT | |
| | ACTGTGAATACTGCTGGGCGAGCATACATTCCCGAGCCGGGCGGGAGTTCCA | |
| | CAAACCGCTGGTGAAGGAGGGAGGCGACCGCCCTCGTCACGTCCCGTTCCGC | |
| | TGGAGCTGA | |
| RET- | ATGGCGAAGGCGACGTCCGGTGCCGCGGGGCTGCGTCTGCTGTTGCTGCTGC | 36 |
| DCC | TGCTGCCGCTGCTAGGCAAAGTGGCATTGGGCCTCTACTTCTCGAGGGATGC | |
| | TTACTGGGAGAAGCTGTATGTGGACCAGGCAGCCGGCACGCCCTTGCTGTAC | |
| | GTCCATGCCCTGCGGGACGCCCCTGAGGAGGTGCCCAGCTTCCGCCTGGGCC | |
| | AGCATCTCTACGGCACGTACCGCACACGGCTGCATGAGAACAACTGGATCTG | |
| | CATCCAGGAGGACACCGGCCTCCTCTACCTTAACCGGAGCCTGGACCATAGC | |
| | TCCTGGGAGAAGCTCAGTGTCCGCAACCGCGGCTTTCCCCTGCTCACCGTCT | |
| | ACCTCAAGGTCTTCCTGTCACCCACATCCCTTCGTGAGGGCGAGTGCCAGTG | |
| | GCCAGGCTGTGCCCGCGTATACTTCTCCTTCTTCAACACCTCCTTTCCAGCC | |
| | TGCAGCTCCCTCAAGCCCCGGGAGCTCTGCTTCCCAGAGACAAGGCCCTCCT | |
| | TCCGCATTCGGGAGAACCGACCCCCAGGCACCTTCCACCAGTTCCGCCTGCT | |
| | GCCTGTGCAGTTCTTGTGCCCCAACATCAGCGTGGCCTACAGGCTCCTGGAG | |
| | GGTGAGGGTCTGCCCTTCCGCTGCGCCCCGGACAGCCTGGAGGTGAGCACGC | |
| | GCTGGGCCCTGGACCGCGAGCAGCGGGAGAAGTACGAGCTGGTGGCCGTGTG | |
| | CACCGTGCACGCCGGCGCGCGCGAGGAGGTGGTGATGGTGCCCTTCCCGGTG | |
| | ACCGTGTACGACGAGGACGACTCGGCGCCCACCTTCCCCGCGGGCGTCGACA | |
| | CCGCCAGCGCCGTGGTGGAGTTCAAGCGGAAGGAGGACACCGTGGTGGCCAC | |
| | GCTGCGTGTCTTCGATGCAGACGTGGTACCTGCATCAGGGGAGCTGGTGAGG | |
| | CGGTACACAAGCACGCTGCTCCCCGGGGACACCTGGGCCCAGCAGACCTTCC | |
| | GGGTGGAACACTGGCCCAACGAGACCTCGGTCCAGGCCAACGGCAGCTTCGT | |
| | GCGGGCGACCGTACATGACTATAGGCTGGTTCTCAACCGGAACCTCTCCATC | |
| | TCGGAGAACCGCACCATGCAGCTGGCGGTGCTGGTCAATGACTCAGACTTCC | |
| | AGGGCCCAGGAGCGGGCGTCCTCTTGCTCCACTTCAACGTGTCGGTGCTGCC | |
| | GGTCAGCCTGCACCTGCCCAGTACCTACTCCCTCTCCGTGAGCAGGAGGGCT | |
| | CGCCGATTTGCCCAGATCGGGAAAGTCTGTGTGGAAAACTGCCAGGCATTCA | |
| | GTGGCATCAACGTCCAGTACAAGCTGCATTCCTCTGGTGCCAACTGCAGCAC | |
| | GCTAGGGGTGGTCACCTCAGCCGAGGACACCTCGGGGATCCTGTTTGTGAAT | |
| | GACACCAAGGCCCTGCGGCGGCCCAAGTGTGCCGAACTTCACTACATGGTGG | |
| | TGGCCACCGACCAGCAGACCTCTAGGCAGGCCCAGGCCCAGCTGCTTGTAAC | |
| | AGTGGAGGGGTCATATGTGGCCGAGGAGGCGGGCTGCCCCCTGTCCTGTGCA | |
| | GTCAGCAAGAGACGGCTGGAGTGTGAGGAGTGTGGCGGCCTGGGCTCCCCAA | |
| | CAGGCAGGTGTGAGTGGAGGCAAGGAGATGGCAAAGGGATCACCAGGAACTT | |
| | CTCCACCTGCTCTCCCAGCACCAAGACCTGCCCCGACGGCCACTGCGATGTT | |
| | GTGGAGACCCAAGACATCAACATTTGCCCTCAGGACTGCCTCCCTATCCCAA | |
| | GCTCCAGTGTCCTCCCTTCGGCTCCCAGAGATGTGGTCCCTGTCTTGGTTTC | |
| | CAGCCGATTTGTCCGTCTCAGCTGGCGCCCACCTGCAGAAGCGAAAGGGAAC | |
| | ATTCAAACTTTCACGGTCTTTTTCTCCAGAGAAGGTGACAACAGGGAACGAG | |
| | CATTGAATACAACACAGCCTGGGTCCCTTCAGCTCACTGTGGGAAACCTGAA | |
| | GCCAGAAGCCATGTACACCTTTCGAGTTGTGGCTTACAATGAATGGGGACCG | |
| | GGAGAGAGTTCTCAACCCATCAAGGTGGCCACACAGCCTGAGTTGCAAGTTC | |
| | CAGGGCCAGTAGAAAACCTGCAAGCTGTATCTACCTCACCTACCTCAATTCT | |
| | TATTACCTGGGAACCCCCTGCCTATGCAAACGGTCCAGTCCAAGGTTACAGA | |
| | TTGTTCTGCACTGAGGTGTCCACAGGAAAAGAACAGAATATAGAGGTTGATG | |
| | GACTATCTTATAAACTGGAAGGCCTGAAAAAATTCACCGAATATAGTCTTCG | |
| | ATTCTTAGCTTATAATCGCTATGGTCCGGGCGTCTCTACTGATGATATAACA | |
| | GTGGTTACACTTTCTGACGTGCCAAGTGCCCCGCCTCAGAACGTCTCCCTGG | |
| | AAGTGGTCAATTCAAGAAGTATCAAAGTTAGCTGGCTGCCTCCTCCATCAGG | |
| | AACACAAAATGGATTTATTACCGGCTATAAAATTCGACACAGAAAGACGACC | |
| | CGCAGGGGTGAGATGGAAACACTGGAGCCAAACAACCTCTGGTACCTATTCA | |
| | CAGGACTGGAGAAAGGAAGTCAGTACAGTTTCCAGGTGTCAGCCATGACAGT | |
| | CAATGGTACTGGACCACCTTCCAACTGGTATACTGCAGAGACTCCAGAGAAT | |
| | GATCTAGATGAATCTCAAGTTCCTGATCAACCAAGCTCTCTTCATGTGAGGC | |
| | CCCAGACTAACTGCATCATCATGAGTTGGACTCCTCCCTTGAACCCAAACAT | |
| | CGTGGTGCGAGGTTATATTATCGGTTATGGCGTTGGGAGCCCTTACGCTGAG | |
| | ACAGTGCGTGTGGACAGCAAGCAGCGATATTATTCCATTGAGAGGTTAGAGT | |
| | CAAGTTCCCATTATGTAATCTCCCTAAAAGCTTTTAACAATGCCGGAGAAGG | |
| | AGTTCCTCTTTATGAAAGTGCCACCACCAGGTCTATAACCGATCCCACTGAC | |
| | CCAGTTGATTATTATCCTTTGCTTGATGATTTCCCCACCTCGGTCCCAGATC | |
| | TCTCCACCCCCATGCTCCCACCAGTAGGTGTACAGGCTGTGGCTCTTACCCA | |
| | TGATGCTGTGAGGGTCAGCTGGGCAGACAACTCTGTCCCTAAGAACCAAAAG | |
| | ACGTCTGAGGTGCGACTTTACACCGTCCGGTGGAGAACCAGCTTTTCTGCAA | |
| | GTGCAAAATACAAGTCAGAAGACACAACATCTCTAAGTTACACAGCAACAGG | |
| | CCTCAAACCAAACACAATGTATGAATTCTCGGTCATGGTAACAAAAAACAGA | |
| | AGGTCCAGTACTTGGAGCATGACTGCACATGCCACCACGTATGAAGCAGCCC | |
| | CCACCTCTGCTCCCAAGGACTTGACAGTCATTACTAGGGAAGGGAAGCCTCG | |
| | TGCCGTCATTGTGAGTTGGCAGCCTCCCTTGGAAGCCAATGGGAAAATTACT | |
| | GCTTACATCTTATTTTATACCTTGGACAAGAACATCCCAATTGATGACTGGA | |
| | TTATGGAAACAATCAGTGGTGATAGGCTTACTCATCAAATCATGGATCTCAA | |
| | CCTTGATACTATGTATTACTTTCGAATTCAAGCACGAAATTCAAAAGGAGTG | |
| | GGGCCACTCTCTGATCCTATCCTCTTCAGGACTCTGAAAGTGGAACACCCTG | |
| | ACAAAATGGCTAATGACCAAGGTCGTCATGGAGATGGAGGTTATTGGCCAGT | |
| | TGATACTAATTTGATTGATAGAAGCACCCTAAATGAGCCGCCAATTGGACAA | |
| | ATGCACCCCCCGCATGGCAGTGTCACTCCTCAGAAGAACAGCAACCTGCTTG | |
| | TGATCATTGTGGTCACCGTTGGTGTCATCACAGTGCTGGTAGTGGTCATCGT | |
| | GGCTGTGATTTGCACCCGACGCTCTTCAGCCCAGCAGAGAAAGAAACGGGCC | |
| | ACCCACAGTGCTGGCAAAAGGAAGGGCAGCCAGAAGGACCTCCGACCCCCTG | |
| | ATCTTTGGATCCATCATGAAGAAATGGAGATGAAAAATATTGAAAAGCCATC | |
| | TGGCACTGACCCTGCAGGAAGGGACTCTCCCATCCAAAGTTGCCAAGACCTC | |
| | ACACCAGTCAGCCACAGCCAGTCAGAAACCCAACTGGGAAGCAAAAGCACCT | |
| | CTCATTCAGGTCAAGACACTGAGGAAGCAGGGAGCTCTATGTCCACTCTGGA | |
| | GAGGTCGCTGGCTGCACGCCGAGCCCCCCGGGCCAAGCTCATGATTCCCATG | |
| | GATGCCCAGTCCAACAATCCTGCTGTCGTGAGCGCCATCCCGGTGCCAACGC | |
| | TAGAAAGTGCCCAGTACCCAGGAATCCTCCCGTCTCCCACCTGTGGATATCC | |
| | CCACCCGCAGTTCACTCTCCGGCCTGTGCCATTCCCAACACTCTCAGTGGAC | |
| | CGAGGTTTCGGAGCAGGAAGAAGTCAGTCAGTGAGTGAAGGACCAACTACCC | |
| | AACAACCACCTATGCTGCCCCCATCTCAGCCTGAGCATTCTAGCAGCGAGGA | |
| | GGCACCAAGCAGAACCATCCCCACAGCTTGTGTTCGACCAACTCACCCACTC | |
| | CGCAGCTTTGCTAATCCTTTGCTACCTCCACCAATGAGTGCAATAGAACCGA | |
| | AAGTCCCTTACACACCACTTTTGTCTCAGCCAGGGCCCACTCTTCCTAAGAC | |
| | CCATGTGAAAACAGCCTCCCTTGGGTTGGCTGGAAAAGCAAGATCCCCTTTG | |
| | CTTCCTGTGTCTGTGCCAACAGCCCCTGAAGTGTCTGAGGAGAGCCACAAAC | |
| | CAACAGAGGATTCAGCCAATGTGTATGAACAGGATGATCTGAGTGAACAAAT | |
| | GGCAAGTTTGGAAGGACTCATGAAGCAGCTTAATGCCATCACAGGCTCAGCC | |
| | TTTTAA | |
| RET- | ATGGCGAAGGCGACGTCCGGTGCCGCGGGGCTGCGTCTGCTGTTGCTGCTGC | 37 |
| ELMO1 | TGCTGCCGCTGCTAGGCAAAGTGGCATTGGGCCTCTACTTCTCGAGGGATGC | |
| | TTACTGGGAGAAGCTGTATGTGGACCAGGCAGCCGGCACGCCCTTGCTGTAC | |
| | GTCCATGCCCTGCGGGACGCCCCTGAGGAGGTGCCCAGCTTCCGCCTGGGCC | |
| | AGCATCTCTACGGCACGTACCGCACACGGCTGCATGAGAACAACTGGATCTG | |
| | CATCCAGGAGGACACCGGCCTCCTCTACCTTAACCGGAGCCTGGACCATAGC | |
| | TCCTGGGAGAAGCTCAGTGTCCGCAACCGCGGCTTTCCCCTGCTCACCGTCT | |
| | ACCTCAAGGTCTTCCTGTCACCCACATCCCTTCGTGAGGGCGAGTGCCAGTG | |
| | GCCAGGCTGTGCCCGCGTATACTTCTCCTTCTTCAACACCTCCTTTCCAGCC | |
| | TGCAGCTCCCTCAAGCCCCGGGAGCTCTGCTTCCCAGAGACAAGGCCCTCCT | |
| | TCCGCATTCGGGAGAACCGACCCCCAGGCACCTTCCACCAGTTCCGCCTGCT | |
| | GCCTGTGCAGTTCTTGTGCCCCAACATCAGCGTGGCCTACAGGCTCCTGGAG | |
| | GGTGAGGGTCTGCCCTTCCGCTGCGCCCCGGACAGCCTGGAGGTGAGCACGC | |
| | GCTGGGCCCTGGACCGCGAGCAGCGGGAGAAGTACGAGCTGGTGGCCGTGTG | |
| | CACCGTGCACGCCGGCGCGCGCGAGGAGGTGGTGATGGTGCCCTTCCCGGTG | |
| | ACCGTGTACGACGAGGACGACTCGGCGCCCACCTTCCCCGCGGGCGTCGACA | |
| | CCGCCAGCGCCGTGGTGGAGTTCAAGCGGAAGGAGGACACCGTGGTGGCCAC | |
| | GCTGCGTGTCTTCGATGCAGACGTGGTACCTGCATCAGGGGAGCTGGTGAGG | |
| | CGGTACACAAGCACGCTGCTCCCCGGGGACACCTGGGCCCAGCAGACCTTCC | |
| | GGGTGGAACACTGGCCCAACGAGACCTCGGTCCAGGCCAACGGCAGCTTCGT | |
| | GCGGGCGACCGTACATGACTATAGGCTGGTTCTCAACCGGAACCTCTCCATC | |
| | TCGGAGAACCGCACCATGCAGCTGGCGGTGCTGGTCAATGACTCAGACTTCC | |
| | AGGGCCCAGGAGCGGGCGTCCTCTTGCTCCACTTCAACGTGTCGGTGCTGCC | |
| | GGTCAGCCTGCACCTGCCCAGTACCTACTCCCTCTCCGTGAGCAGGAGGGCT | |
| | CGCCGATTTGCCCAGATCGGGAAAGTCTGTGTGGAAAACTGCCAGGCATTCA | |
| | GTGGCATCAACGTCCAGTACAAGCTGCATTCCTCTGGTGCCAACTGCAGCAC | |
| | GCTAGGGGTGGTCACCTCAGCCGAGGACACCTCGGGGATCCTGTTTGTGAAT | |
| | GACACCAAGGCCCTGCGGCGGCCCAAGTGTGCCGAACTTCACTACATGGTGG | |
| | TGGCCACCGACCAGCAGACCTCTAGGCAGGCCCAGGCCCAGCTGCTTGTAAC | |
| | AGTGGAGGGGTCATATGTGGCCGAGGAGGCGGGCTGCCCCCTGTCCTGTGCA | |
| | GTCAGCAAGAGACGGCTGGAGTGTGAGGAGTGTGGCGGCCTGGGCTCCCCAA | |
| | CAGGCAGGTGTGAGTGGAGGCAAGGAGATGGCAAAGGGATCACCAGGAACTT | |
| | CTCCACCTGCTCTCCCAGCACCAAGACCTGCCCCGACGGCCACTGCGATGTT | |
| | GTGGAGACCCAAGACATCAACATTTGCCCTCAGGACTGCCTCCGGGGCAGCA | |
| | TTGTTGGGGGACACGAGCCTGGGGAGCCCCGGGGGATTAAAGCTGGCTATGG | |
| | CACCTGCAACTGCTTCCCTGAGGAGGAGAAGTGCTTCTGCGAGCCCGAAGAC | |
| | ATCCAGGATCCACTGTGCGACGAGCTGTGCCGCACGGTGATCGCAGCCGCTG | |
| | TCCTCTTCTCCTTCATCGTCTCGGTGCTGCTGTCTGCCTTCTGCATCCACTG | |
| | CTACCACAAGTTTGCCCACAAGCCACCCATCTCCTCAGCTGAGATGACCTTC | |
| | CGGAGGCCCGCCCAGGCCTTCCCGGTCAGCTACTCCTCTTCCGGTGCCCGCC | |
| | GGCCCTCGCTGGACTCCATGGAGAACCAGGTCTCCGTGGATGCCTTCAAGAT | |
| | CCTGAATCATGTCAACCCTGCCATGGACTTCACGCAGACTCCACCTGGGATG | |
| | TTGGCTCTGGACAACATGCTGTACTTTGCCAAGCACCACCAAGATGCCTACA | |
| | TCCGGATTGTGCTTGAGAACAGTAGTCGAGAAGACAAGCATGAATGTCCCTT | |
| | TGGCCGCAGTAGTATAGAGCTGACCAAGATGCTATGTGAGATCTTGAAAGTG | |
| | GGCGAGTTGCCTAGTGAGACCTGCAACGACTTCCACCCGATGTTCTTCACCC | |
| | ACGACAGATCCTTTGAGGAGTTTTTCTGCATCTGTATCCAGCTCCTGAACAA | |
| | GACATGGAAGGAAATGAGGGCAACTTCTGAAGACTTCAACAAGGTAATGCAG | |
| | GTGGTGAAGGAGCAGGTTATGAGAGCACTTACAACCAAGCCTAGCTCCCTGG | |
| | ACCAGTTCAAGAGCAAACTGCAGAACCTGAGCTACACTGAGATCCTGAAAAT | |
| | CCGCCAGTCCGAGAGGATGAACCAGGAAGATTTCCAGTCCCGCCCGATTTTG | |
| | GAACTAAAGGAGAAGATTCAGCCAGAAATCTTAGAGCTGATCAAACAGCAAC | |
| | GCCTGAACCGCCTTGTGGAAGGGACCTGCTTTAGGAAACTCAATGCCCGGCG | |
| | GAGGCAAGACAAGTTTTGGTATTGTCGGCTTTCGCCAAATCACAAAGTCCTG | |
| | CATTACGGAGACTTAGAAGAGAGTCCTCAGGGAGAAGTGCCCCACGATTCCT | |
| | TGCAGGACAAACTGCCGGTGGCAGATATCAAAGCCGTGGTGACGGGAAAGGA | |
| | CTGCCCTCATATGAAAGAGAAAGGTGCCCTTAAACAAAACAAGGAGGTGCTT | |
| | GAACTCGCTTTCTCCATCTTGTATGACTCAAACTGCCAACTGAACTTCATCG | |
| | CTCCTGACAAGCATGAGTACTGTATCTGGACGGATGGACTGAATGCGCTACT | |
| | CGGGAAGGACATGATGAGCGACCTGACGCGGAATGACCTGGACACCCTGCTC | |
| | AGCATGGAAATCAAGCTCCGCCTCCTGGACCTGGAAAACATCCAGATCCCTG | |
| | ACGCACCTCCGCCGATTCCCAAGGAGCCCAGCAACTATGACTTCGTCTATGA | |
| | CTGTAACTGA | |
| RET- | ATGGCGAAGGCGACGTCCGGTGCCGCGGGGCTGCGTCTGCTGTTGCTGCTGC | 38 |
| WDFY4 | TGCTGCCGCTGCTAGGCAAAGTGGCATTGGGCCTCTACTTCTCGAGGGATGC | |
| | TTACTGGGAGAAGCTGTATGTGGACCAGGCAGCCGGCACGCCCTTGCTGTAC | |
| | GTCCATGCCCTGCGGGACGCCCCTGAGGAGGTGCCCAGCTTCCGCCTGGGCC | |
| | AGCATCTCTACGGCACGTACCGCACACGGCTGCATGAGAACAACTGGATCTG | |
| | CATCCAGGAGGACACCGGCCTCCTCTACCTTAACCGGAGCCTGGACCATAGC | |
| | TCCTGGGAGAAGCTCAGTGTCCGCAACCGCGGCTTTCCCCTGCTCACCGTCT | |
| | ACCTCAAGGTCTTCCTGTCACCCACATCCCTTCGTGAGGGCGAGTGCCAGTG | |
| | GCCAGGCTGTGCCCGCGTATACTTCTCCTTCTTCAACACCTCCTTTCCAGCC | |
| | TGCAGCTCCCTCAAGCCCCGGGAGCTCTGCTTCCCAGAGACAAGGCCCTCCT | |
| | TCCGCATTCGGGAGAACCGACCCCCAGGCACCTTCCACCAGTTCCGCCTGCT | |
| | GCCTGTGCAGTTCTTGTGCCCCAACATCAGCGTGGCCTACAGGCTCCTGGAG | |
| | GGTGAGGGTCTGCCCTTCCGCTGCGCCCCGGACAGCCTGGAGGTGAGCACGC | |
| | GCTGGGCCCTGGACCGCGAGCAGCGGGAGAAGTACGAGCTGGTGGCCGTGTG | |
| | CACCGTGCACGCCGGCGCGCGCGAGGAGGTGGTGATGGTGCCCTTCCCGGTG | |
| | ACCGTGTACGACGAGGACGACTCGGCGCCCACCTTCCCCGCGGGCGTCGACA | |
| | CCGCCAGCGCCGTGGTGGAGTTCAAGCGGAAGGAGGACACCGTGGTGGCCAC | |
| | GCTGCGTGTCTTCGATGCAGACGTGGTACCTGCATCAGGGGAGCTGGTGAGG | |
| | CGGTACACAAGCACGCTGCTCCCCGGGGACACCTGGGCCCAGCAGACCTTCC | |
| | GGGTGGAACACTGGCCCAACGAGACCTCGGTCCAGGCCAACGGCAGCTTCGT | |
| | GCGGGCGACCGTACATGACTATAGGCTGGTTCTCAACCGGAACCTCTCCATC | |
| | TCGGAGAACCGCACCATGCAGCTGGCGGTGCTGGTCAATGACTCAGACTTCC | |
| | AGGGCCCAGGAGCGGGCGTCCTCTTGCTCCACTTCAACGTGTCGGTGCTGCC | |
| | GGTCAGCCTGCACCTGCCCAGTACCTACTCCCTCTCCGTGAGCAGGAGGGCT | |
| | CGCCGATTTGCCCAGATCGGGAAAGTCTGTGTGGAAAACTGCCAGGCATTCA | |
| | GTGGCATCAACGTCCAGTACAAGCTGCATTCCTCTGGTGCCAACTGCAGCAC | |
| | GCTAGGGGTGGTCACCTCAGCCGAGGACACCTCGGGGATCCTGTTTGTGAAT | |
| | GACACCAAGGCCCTGCGGCGGCCCAAGTGTGCCGAACTTCACTACATGGTGG | |
| | TGGCCACCGACCAGCAGACCTCTAGGCAGGCCCAGGCCCAGCTGCTTGTAAC | |
| | AGTGGAGGGGTCATATGTGGCCGAGGAGGCGGGCTGCCCCCTGTCCTGTGCA | |
| | GTCAGCAAGAGACGGCTGGAGTGTGAGGAGTGTGGCGGCCTGGGCTCCCCAA | |
| | CAGGCAGGTGTGAGTGGAGGCAAGGAGATGGCAAAGGGATCACCAGGAACTT | |
| | CTCCACCTGCTCTCCCAGCACCAAGACCTGCCCCGACGGCCACTGCGATGTT | |
| | GTGGAGACCCAAGACATCAACATTTGCCCTCAGGACTGCCTCCGGGGCAGCA | |
| | TTGTTGGGGGACACGAGCCTGGGGAGCCCCGGGGGATTAAAGCTGGCTATGG | |
| | CACCTGCAACTGCTTCCCTGAGGAGGAGAAGTGCTTCTGCGAGCCCGAAGAC | |
| | ATCCAGGATCCACTGTGCGACGAGCTGTGCCGCACGGTGATCGCAGCCGCTG | |
| | TCCTCTTCTCCTTCATCGTCTCGGTGCTGCTGTCTGCCTTCTGCATCCACTG | |
| | CTACCACAAGTTTGCCCACAAGCCACCCATCTCCTCAGCTGAGATGACCTTC | |
| | CGGAGGCCCGCCCAGGCCTTCCCGGTCAGCTACTCCTCTTCCGGTGCCCGCC | |
| | GGCCCTCGCTGGACTCCATGGAGAACCAGGTCTCCGTGGATGCCTTCAAGAT | |
| | CCTGGAGGATCCAAAGTGGGAATTCCCTCGGAAGAACTTGGTTCTTGGAAAA | |
| | ACTCTAGGAGAAGGCGAATTTGGAAAAGTGGTCAAGGCAACGGCCTTCCATC | |
| | TGAAAGGCAGAGCAGGGTACACCACGGTGGCCGTGAAGATGCTGAAAGAGAA | |
| | CGCCTCCCCGAGTGAGCTGCGAGACCTGCTGTCAGAGTTCAACGTCCTGAAG | |
| | CAGGTCAACCACCCACATGTCATCAAATTGTATGGGGCCTGCAGCCAGGATG | |
| | GCCCGCTCCTCCTCATCGTGGAGTACGCCAAATACGGCTCCCTGCGGGGCTT | |
| | CCTCCGCGAGAGCCGCAAAGTGGGGCCTGGCTACCTGGGCAGTGGAGGCAGC | |
| | CGCAACTCCAGCTCCCTGGACCACCCGGATGAGCGGGCCCTCACCATGGGCG | |
| | ACCTCATCTCATTTGCCTGGCAGATCTCACAGGGGATGCAGTATCTGGCCGA | |
| | GATGAAGCTCGTTCATCGGGACTTGGCAGCCAGAAACATCCTGGTAGCTGAG | |
| | GGGCGGAAGATGAAGATTTCGGATTTCGGCTTGTCCCGAGATGTTTATGAAG | |
| | AGGATTCCTACGTGAAGAGGAGCCAGGATTTTGTGTCATGTATAGAGAACTA | |
| | CAGAAGAAGAGGACAAGAGCTATATGCATCTTTATACAAAGACCATGTGCAA | |
| | AGGCGAAAATGTGGCAACATCAAGGCAGCCAACGCCTGGGCCAGGATCCAGG | |
| | AGCAGCTTTTTGGGGAGCTGGGCTTGTGGAGCCAGGGGGAAGAAACCAAGCC | |
| | CTGTTCCCCATGGGAACTCGACTGGAGAGAAGGACCAGCTCGAATGAGGAAA | |
| | CGCATCAAACGCTTGTCTCCTTTGGAGGCCCTGAGCTCAGGAAGGCACAAGG | |
| | AAAGCCAAGACAAAAATGATCATATTTCTCAAACAAATGCTGAAAACCAAGA | |
| | TGAACTGACACTGAGGGAGGCTGAGGGCGAGCCGGACGAGGTGGGGGTGGAC | |
| | TGCACCCAGCTGACCTTCTTCCCAGCCTTACACGAAAGTCTGCACTCAGAAG | |
| | ACTTCTTGGAACTGTGTCGGGAAAGACAAGTTATTTTACAAGAGCTTCTTGA | |
| | TAAAGAAAAGGTGACGCAGAAGTTCTCCCTGGTGATTGTGCAGGGCCACCTG | |
| | GTGTCAGAAGGGGTCCTGCTTTTTGGCCACCAACACTTCTACATCTGCGAGA | |
| | ACTTCACACTGTCTCCCACGGGTGATGTCTACTGTACCCGTCACTGCTTATC | |
| | CAACATCAGCGATCCGTTCATTTTCAACCTGTGCAGCAAAGACAGGTCCACT | |
| | GACCATTACTCGTGCCAGTGCCACAGCTACGCTGACATGCGGGAGCTACGGC | |
| | AGGCTCGCTTCCTCCTGCAGGACATCGCCCTGGAGATCTTCTTCCACAATGG | |
| | ATATTCCAAGTTTCTTGTCTTCTACAACAATGATCGGAGTAAGGCCTTTAAA | |
| | AGCTTCTGCTCTTTCCAACCCAGCCTGAAGGGGAAAGCCACCTCGGAGGACA | |
| | CCCTCAGTCTAAGGAGATACCCCGGCTCTGACAGGATCATGCTGCAGAAGTG | |
| | GCAGAAAAGGGACATCAGCAATTTTGAGTATCTCATGTACCTCAACACCGCG | |
| | GCTGGGAGAACCTGCAATGACTACATGCAGTACCCAGTGTTCCCCTGGGTCC | |
| | TCGCAGACTACACCTCAGAGACATTGAACTTGGCAAATCCGAAGATTTTCCG | |
| | GGATCTTTCAAAGCCCATGGGGGCTCAGACCAAGGAAAGGAAGCTGAAATTT | |
| | ATCCAGAGGTTTAAAGAAGTTGAGAAAACTGAAGGAGACATGACTGTCCAGT | |
| | GCCACTACTACACCCACTACTCCTCGGCCATCATCGTGGCCTCCTACCTGGT | |
| | CCGGATGCCACCCTTCACCCAGGCCTTCTGCGCTCTGCAGGGCGGAAGCTTC | |
| | GACGTGGCAGACAGAATGTTCCACAGTGTGAAGAGCACGTGGGAGTCGGCCT | |
| | CCAGAGAGAACATGAGTGACGTCAGGGAGCTGACCCCAGAGTTCTTCTACCT | |
| | GCCTGAGTTCTTAACCAACTGCAACGGGGTAGAGTTCGGCTGCATGCAGGAC | |
| | GGGACTGTGCTAGGAGACGTGCAGCTCCCTCCCTGGGCTGATGGGGACCCTC | |
| | GGAAATTCATCAGCCTGCACAGAAAGGCCCTGGAAAGTGACTTTGTCAGTGC | |
| | CAACCTCCACCATTGGATAGACCTTATTTTTGGGTACAAGCAGCAGGGGCCA | |
| | GCCGCAGTGGATGCTGTTAATATCTTCCACCCCTACTTCTACGGTGACAGAA | |
| | TGGACCTCAGCAGCATCACTGACCCCCTCATCAAAAGCACCATCCTGGGGTT | |
| | TGTCAGCAACTTTGGACAGGTGCCCAAACAGCTCTTTACCAAACCTCACCCA | |
| | GCCAGGACTGCAGCAGGGAAGCCTCTGCCTGGAAAGGATGTCTCCACCCCCG | |
| | TGAGCCTGCCTGGCCACCCACAGCCCTTTTTCTACAGCCTGCAGTCGCTGAG | |
| | GCCCTCCCAGGTCACGGTCAAAGATATGTACCTCTTTTCTCTAGGCTCAGAG | |
| | TCCCCCAAAGGGGCCATTGGCCACATTGTCTCTACTGAGAAGACCATTCTGG | |
| | CTGTAGAGAGGAACAAAGTGCTGCTGCCTCCTCTCTGGAACAGGACCTTCAG | |
| | CTGGGGCTTTGATGACTTCAGCTGCTGCTTGGGGAGCTACGGCTCCGACAAG | |
| | GTCCTGATGACATTCGAGAACCTGGCTGCCTGGGGCCGCTGTCTGTGCGCCG | |
| | TGTGCCCATCCCCAACAACGATTGTCACCTCTGGGACCAGCACTGTGGTGTG | |
| | TGTGTGGGAGCTCAGCATGACCAAAGGCCGCCCGAGGGGCTTGCGCCTCCGG | |
| | CAGGCCTTGTATGGACACACACAGGCTGTCACGTGCCTGGCAGCGTCAGTCA | |
| | CCTTCAGCCTCCTGGTGAGCGGCTCCCAGGACTGCACCTGTATCCTGTGGGA | |
| | TCTGGACCACCTCACCCACGTGACCCGCCTGCCCGCCCATCGGGAAGGCATC | |
| | TCAGCCATCACCATCAGTGACGTCTCAGGCACCATTGTCTCCTGTGCGGGAG | |
| | CACACTTGTCCCTGTGGAATGTCAATGGACAGCCCCTGGCCAGCATCACCAC | |
| | AGCCTGGGGCCCAGAAGGAGCCATAACCTGCTGCTGCCTGATGGAGGGCCCA | |
| | GCATGGGACACAAGCCAGATCATCATCACCGGGAGTCAAGACGGCATGGTCC | |
| | GGGTTTGGAAGACTGAGGATGTGAAGATGTCTGTTCCTGGACGGCCAGCAGG | |
| | AGAGGAGCCCCCGGCTCAGCCTCCAAGCCCAAGAGGCCACAAGTGGGAGAAG | |
| | AACCTGGCCTTGAGTCGAGAGCTGGACGTTAGCATTGCTTTGACAGGGAAGC | |
| | CCAGCAAAACCAGCCCCGCAGTGACTGCTCTGGCCGTGTCCAGAAACCACAC | |
| | CAAACTCCTGGTTGGTGATGAGAGGGGGAGAATATTCTGCTGGTCTGCAGAT | |
| | GGGTAG | |
| ANKRD26- | ATGAAGAAGATTTTTAGTAAGAAGGGCGAGTCGCCCTTGGGCTCCTTCGCGC | 39 |
| RET | GGCGGCAGAGGAGCAGCGCGGGAGGCGGGGGCGAGCCGGGGGAGGGCGCCTA | |
| | CTCGCAGCCCGGCTACCACGTCCGAGACCGAGATCTCGGCAAGATCCACAAA | |
| | GCTGCCAGCGCGGGTAATGTGGCGAAAGTGCAGCAGATCCTTTTGCTCAGGA | |
| | AGAATGGCTTGAACGATAGAGACAAGATGAACAGGACGGCTCTACATTTGGC | |
| | CTGTGCCAATGGTCATCCAGAAGTAGTAACTCTCCTGGTGGACAGAAAATGC | |
| | CAGCTCAATGTCTGTGACAACGAAAACAGGACAGCTCTGATGAAGGCTGTAC | |
| | AATGCCAGGAAGAGAAATGTGCAACTATTCTGCTAGAACATGGTGCTGATCC | |
| | AAATCTTGCGGATGTCCATGGCAACACTGCTCTTCACTATGCTGTCTATAAT | |
| | GAGGACATATCAGTAGCAACAAAGCTGCTTTTGTATGATGCAAATATTGAAG | |
| | CAAAAAACAAGGATGACCTCACACCACTTTTACTTGCAGTAAGTGGAAAAAA | |
| | GCAGCAAATGGTGGAATTTTTAATAAAGAAAAAAGCAAATGTAAATGCAGTA | |
| | GATAAGTTGGAAAGCAGTCACCAACTAATTTCAGAATATAAAGAAGAAAGGA | |
| | TACCTAAACATTCTTCTCAAAATAGTAATTCAGTGGATGAAAGCTCTGAAGA | |
| | CTCCTTAAGCAGGCTTTCTGGCAAACCGGGTGTTGATGATTCATGGCCTACC | |
| | TCAGATGACGAAGACCTCAATTTTGATACTAAGAATGTCCCAAAACCAAGCT | |
| | TAGCAAAGCTAATGACTGCTTCTCAGCAATCCAGGAAAAATTTAGAAGCAAC | |
| | ATATGGCACTGTGAGAACAGGAAATAGAACTTTGTTTGAGGATAGAGATTCC | |
| | GATAGTCAAGATGAAGTTGTGGTTGAAAGCCTTCCTACAACATCAATCAAAG | |
| | TCCAGTGCTTTTCTCATCCTACCTATCAATCACCTGACCTTCTTCCAAAACC | |
| | TTCCCACAAGTCGTTAGCAAACCCTGGTCTTATGAAGGAAGAACCAACAAAG | |
| | CCAGGCATTGCAAAAAAAGAAAATGGTATTGATATTATTGAAAGTGCTCCAC | |
| | TAGAGCAAACAAATAATGACAATTTGACTTATGTTGATGAAGTGCACAAAAA | |
| | TAATAGAAGTGATATGATGTCCGCATTAGGATTAGGACAAGAGGAAGATATA | |
| | GAATCACCTTGGGATTCTGAGAGTATCTCTGAGAATTTTCCACAGAAGTATG | |
| | TTGATCCTTTAGCTGGGGCTGCAGACGGAAAAGAAAAAAATATAGGAAATGA | |
| | ACAAGCAGAAGATGTGTTTTATATACCTTCTTGCATGAGTGGATCAAGAAAC | |
| | TTTAAGATGGCTAAACTAGAGGATACAAGAAATGTAGGCATGCCAGTAGCCC | |
| | ACATGGAGTCTCCTGAGAGATATCTTCACTTGAAGCCTACCATTGAAATGAA | |
| | AGATTCTGTTCCAAATAAAGCAGGAGGAATGAAGGATGTACAAACATCCAAA | |
| | GCAGCTGAACATGACTTAGAAGTAGCATCAGAAGAAGAGCAAGAAAGGGAAG | |
| | GGAGTGAAAATAACCAGCCACAGGTTGAAGAAGAAAGGAAAAAACACAGAAA | |
| | TAATGAAATGGAAGTATCAGCAAACATACATGATGGTGCTACTGATGATGCT | |
| | GAAGATGATGATGATGATGATGGATTAATTCAAAAAAGAAAGAGTGGAGAAA | |
| | CTGATCATCAGCAATTTCCCAGGAAGGAAAATAAAGAGTATGCTAGTAGTGG | |
| | TCCTGCCTTGCAAATGAAGGAAGTAAAGAGCACTGAAAAAGAAAAACGGACC | |
| | TCGAAAGAATCTGTGAATTCACCAGTGTTTGGGAAGGCCAGTTTACTAACTG | |
| | GTGGCCTGCTACAAGTGGATGATGACAGCAGTTTAAGTGAAATAGATGAGGA | |
| | TGAAGGAAGGCCTACTAAGAAAACATCTAATGAAAAGAACAAGGTCAAAAAC | |
| | CAAATACAGTCTATGGATGATGTTGATGACTTAACTCAGTCATCTGAAACAG | |
| | CCTCAGAGGATTGTGAGCTACCCCACTCTAGTTACAAGAATTTTATGTTGCT | |
| | CATTGAACAACTTGGAATGGAGTGTAAAGATTCTGTTAGCCTATTGAAAATC | |
| | CAGGATGCAGCTCTTTCATGTGAAAGATTATTAGAACTTAAAAAAAATCACT | |
| | GTGAACTACTTACAGTAAAAATTAAAAAAATGGAAGACAAGGTTAATGTACT | |
| | ACAAAGGGAGCTATCTGAAACAAAAGAAATAAAATCACAGTTAGAGCATCAA | |
| | AAAGTTGAATGGGAACGAGAACTGTGCTCTTTGAGATTTAGCTTAAACCAAG | |
| | AAGAAGAGAAGAGAAGAAATGCTGATACGTTGTATGAAAAAATTAGGGAACA | |
| | GTTAAGAAGAAAAGAAGAGCAATATAGGAAAGAAGTTGAAGTGAAACAACAG | |
| | CTTGAACTGAGTCTCCAAACACTGGAGATGGAATTGAGGACTGTAAAAAGTA | |
| | ATTTGAATCAGGTCGTTCAAGAGCGAAATGACGCTCAGAGGCAACTTTCTCG | |
| | AGAACAGAATGCCAGAATGTTACAAGATGGAATTCTGACCAATCACCTTTCC | |
| | AAACAAAAGGAGATTGAAATGGCTCAAAAGAAAATGAATTCTGAGAATTCTC | |
| | ATAGTCATGAAGAAGAAAAAGACCTATCGCATAAAAATAGCATGTTGCAGGA | |
| | AGAAATTGCTATGCTAAGACTAGAAATAGACACAATAAAAAATCAAAACCAG | |
| | GAAAAAGAAAAGAAATGTTTTGAGGACCTTAAAATTGTAAAAGAAAAGAATG | |
| | AAGACCTTCAGAAGACTATAAAACAGAATGAGGAAACATTAACACAAACAAT | |
| | ATCCCAGTATAATGGACGGCTTAGTGTTCTGACAGCTGAGAATGCAATGCTA | |
| | AATTCTAAACTGGAGAATGAAAAGCAAAGCAAGGAAAGACTGGAAGCAGAAG | |
| | TTGAATCATACCATTCTAGATTGGCTGCTGCTATACATGATCGTGATCAAAG | |
| | TGAGACATCAAAAAGAGAACTAGAACTTGCTTTCCAGAGAGCAAGAGATGAA | |
| | TGTTCTCGTTTACAGGACAAAATGAATTTTGATGTGTCTAACCTAAAAGATA | |
| | ACAATGAGATTCTTTCTCAACAACTATTTAAAACTGAAAGTAAACTCAATAG | |
| | CCTAGAAATTGAGTTCCATCACACGAGAGATGCCCTCAGAGAAAAGACTTTG | |
| | GGTTTAGAACGGGTACAAAAGGACCTAAGCCAAACACAGTGTCAAATGAAGG | |
| | AAATGGAACAAAAGTATCAAAATGAACAAGTTAAAGTGAATAAATACATTGG | |
| | AAAGCAGGAGTCTGTAGAGGAGAGATTGTCTCAACTACAAAGTGAGAATATG | |
| | TTGCTTCGACAACAACTGGATGATGCCCACAACAAGGCTGACAATAAAGAGA | |
| | AGACAGTGATTAATATCCAAGACCAGTTTCATGCTATTGTGCAAAAACTTCA | |
| | AGCTGAGAGTGAAAAGCAAAGTCTTCTGCTAGAAGAAAGAAATAAGGAGTTA | |
| | ATCAGTGAATGTAATCACTTAAAAGAAAGACAGTATCAATATGAAAATGAAA | |
| | AGGCAGAAAGAGAAGTTGTTGTGAGACAACTTCAACAAGAACTAGCTGATAC | |
| | CCTAAAAAAACAATCTATGTCAGAGGCTTCACTGGAGGTTACGTCACGTTAT | |
| | CGTATTAATTTAGAAGATGAGACACAGGATTTAAAGAAGAAATTAGGTCAAA | |
| | TCAGAAATCAATTGCAAGAAGCACAGGATCGACATACAGAAGCTGTCAGATG | |
| | TGCTGAGAAGATGCAAGATCACAAGCAAAAGCTTGAAAAAGATAATGCCAAG | |
| | TTAAAAGTTACAGTCAAAAAGCAAATGGACAAAATTGAGGAGCTTCAGAAAA | |
| | ACCTGTTAAATGCAAATTTGTCTGAAGATGAAAAGGAACAATTAAAGAAACT | |
| | TATGGAATTAAAACAGTCACTGGAATGTAATTTGGATCAAGAAATGAAGAAA | |
| | AATGTTGAATTAGAAAGAGAGATAACTGGATTTAAGAACCTCTTAAAAATGA | |
| | CAAGAAAGAAGTTAAATGAATATGAAAATGGAGAATTTAGTTTCCATGGAGA | |
| | TTTAAAAACTAGTCAATTTGAAATGGATATTCAGATTAATAAGCTAAAACAT | |
| | AAGGAGGATCCAAAGTGGGAATTCCCTCGGAAGAACTTGGTTCTTGGAAAAA | |
| | CTCTAGGAGAAGGCGAATTTGGAAAAGTGGTCAAGGCAACGGCCTTCCATCT | |
| | GAAAGGCAGAGCAGGGTACACCACGGTGGCCGTGAAGATGCTGAAAGAGAAC | |
| | GCCTCCCCGAGTGAGCTGCGAGACCTGCTGTCAGAGTTCAACGTCCTGAAGC | |
| | AGGTCAACCACCCACATGTCATCAAATTGTATGGGGCCTGCAGCCAGGATGG | |
| | CCCGCTCCTCCTCATCGTGGAGTACGCCAAATACGGCTCCCTGCGGGGCTTC | |
| | CTCCGCGAGAGCCGCAAAGTGGGGCCTGGCTACCTGGGCAGTGGAGGCAGCC | |
| | GCAACTCCAGCTCCCTGGACCACCCGGATGAGCGGGCCCTCACCATGGGCGA | |
| | CCTCATCTCATTTGCCTGGCAGATCTCACAGGGGATGCAGTATCTGGCCGAG | |
| | ATGAAGCTCGTTCATCGGGACTTGGCAGCCAGAAACATCCTGGTAGCTGAGG | |
| | GGCGGAAGATGAAGATTTCGGATTTCGGCTTGTCCCGAGATGTTTATGAAGA | |
| | GGATTCCTACGTGAAGAGGAGCCAGGGTCGGATTCCAGTTAAATGGATGGCA | |
| | ATTGAATCCCTTTTTGATCATATCTACACCACGCAAAGTGATGTATGGTCTT | |
| | TTGGTGTCCTGCTGTGGGAGATCGTGACCCTAGGGGGAAACCCCTATCCTGG | |
| | GATTCCTCCTGAGCGGCTCTTCAACCTTCTGAAGACCGGCCACCGGATGGAG | |
| | AGGCCAGACAACTGCAGCGAGGAGATGTACCGCCTGATGCTGCAATGCTGGA | |
| | AGCAGGAGCCGGACAAAAGGCCGGTGTTTGCGGACATCAGCAAAGACCTGGA | |
| | GAAGATGATGGTTAAGAGGAGAGACTACTTGGACCTTGCGGCGTCCACTCCA | |
| | TCTGACTCCCTGATTTATGACGACGGCCTCTCAGAGGAGGAGACACCGCTGG | |
| | TGGACTGTAATAATGCCCCCCTCCCTCGAGCCCTCCCTTCCACATGGATTGA | |
| | AAACAAACTCTATGGTAGAATTTCCCATGCATTTACTAGATTCTAG | |
| CLIP1- | ATGAGTATGCTAAAGCCAAGTGGGCTTAAGGCCCCCACCAAGATCCTGAAGC | 40 |
| RET | CTGGAAGCACAGCTCTGAAGACACCTACGGCTGTTGTAGCTCCAGTAGAAAA | |
| | AACCATATCCAGTGAAAAAGCATCAAGCACTCCATCATCTGAGACTCAGGAG | |
| | GAATTTGTGGATGACTTTCGAGTTGGGGAGCGAGTTTGGGTGAATGGAAATA | |
| | AGCCTGGATTTATCCAGTTTCTTGGAGAAACCCAGTTTGCACCAGGCCAGTG | |
| | GGCTGGAATTGTTTTAGATGAACCCATAGGCAAGAACGATGGTTCGGTGGCA | |
| | GGAGTTCGGTATTTCCAGTGTGAACCTTTAAAGGGCATATTTACCCGACCTT | |
| | CAAAGTTAACAAGGAAGGTGCAAGCAGAAGATGAAGCTAATGGCCTGCAGAC | |
| | AACGCCCGCCTCCCGAGCTACTTCACCGCTGTGCACTTCTACGGCCAGCATG | |
| | GTGTCTTCCTCCCCCTCCACCCCTTCAAACATCCCTCAGAAACCATCACAGC | |
| | CAGCAGCAAAGGAACCTTCAGCTACGCCTCCGATCAGCAACCTTACAAAAAC | |
| | TGCCAGTGAATCTATCTCCAACCTTTCAGAGGCTGGCTCAATCAAGAAAGGA | |
| | GAAAGAGAGCTCAAAATCGGAGACAGAGTATTGGTTGGTGGCACTAAGGCTG | |
| | GTGTAGTCCGGTTTCTTGGGGAGACCGACTTTGCCAAGGGGGAGTGGTGTGG | |
| | CGTGGAGTTAGATGAGCCACTTGGGAAGAATGATGGCGCTGTTGCTGGAACA | |
| | AGGTATTTTCAGTGTCAACCCAAATATGGCTTGTTCGCTCCTGTCCACAAAG | |
| | TTACCAAGATTGGCTTCCCTTCCACTACACCAGCCAAAGCCAAGGCCAACGC | |
| | AGTGAGGCGAGTGATGGCGACCACGTCCGCCAGCCTGAAGCGCAGCCCTTCT | |
| | GCCTCTTCCCTCAGCTCCATGAGCTCAGTGGCCTCCTCTGTGAGCAGCAGGC | |
| | CCAGTCGGACAGGACTATTGACTGAAACCTCCTCCCGTTACGCCAGGAAGAT | |
| | CTCCGGTACCACTGCCCTCCAGGAGGCCCTGAAGGAGAAGCAGCAGCACATT | |
| | GAGCAGCTGCTGGCGGAACGGGATCTGGAGAGGGCGGAGGTGGCCAAGGCCA | |
| | CGAGCCACGTGGGGGAGATAGAGCAGGAGCTAGCTCTGGCCCGGGACGGACA | |
| | TGACCAGCATGTCCTGGAATTGGAAGCCAAAATGGACCAGCTGCGAACAATG | |
| | GTGGAAGCTGCTGACAGGGAGAAGGTGGAGCTTCTCAACCAGCTTGAAGAGG | |
| | AGAAAAGGAAGGTTGAGGACCTTCAGTTCCGGGTTGAAGAAGAATCAATTAC | |
| | CAAAGGTGATCTTGAGACGCAGACCAAACTGGAGCATGCCCGCATTAAGGAG | |
| | CTTGAACAGAGCCTGCTCTTTGAAAAGACCAAAGCTGACAAACTCCAGAGGG | |
| | AGTTAGAAGACACTAGGGTGGCTACAGTTTCAGAAAAGTCACGTATAATGGA | |
| | ACTGGAGAAAGACCTAGCATTGAGAGTACAGGAAGTAGCTGAGCTCCGAAGA | |
| | AGGCTAGAGTCCAATAAGCCTGCTGGGGATGTGGACATGTCACTTTCCCTTT | |
| | TGCAAGAGATAAGCTCTTTGCAAGAAAAGTTAGAAGTCACCCGTACTGACCA | |
| | CCAGAGAGAAATAACTTCTCTGAAGGAGCATTTTGGAGCCCGGGAAGAAACT | |
| | CATCAGAAGGAGATAAAGGCTCTGTATACCGCCACGGAAAAGCTTTCCAAAG | |
| | AGAACGAGTCATTGAAAAGCAAGCTGGAGCATGCCAACAAAGAGAACTCAGA | |
| | TGTGATAGCTCTATGGAAGTCCAAACTGGAGACTGCCATCGCATCCCACCAG | |
| | CAGGCGATGGAAGAACTGAAGGTATCTTTCAGCAAAGGGCTTGGAACAGAGA | |
| | CGGCAGAATTTGCTGAACTAAAAACACAAATAGAGAAAATGAGACTAGATTA | |
| | CCAACACGAAATAGAAAATTTGCAGAATCAACAAGACTCTGAACGGGCTGCC | |
| | CATGCTAAAGAGATGGAAGCCTTGAGGGCTAAACTGATGAAAGTTATTAAAG | |
| | AAAAGGAAAACAGTCTGGAAGCCATCAGGTCGAAACTGGACAAAGCAGAAGA | |
| | CCAGCATCTCGTAGAAATGGAAGACACGTTAAACAAATTACAGGAAGCTGAA | |
| | ATAAAGGTAAAGGAGCTAGAGGTACTGCAAGCCAAATGCAATGAACAAACCA | |
| | AGGTTATTGATAATTTTACATCACAGCTCAAGGCTACTGAAGAAAAGCTCTT | |
| | GGATCTTGATGCACTTCGGAAAGCCAGTTCCGAAGGTAAATCGGAAATGAAG | |
| | AAACTTAGACAGCAGCTTGAGGCAGCTGAGAAACAGATTAAACATTTAGAGA | |
| | TTGAAAAGAATGCTGAAAGTAGCAAGGCTAGTAGCATTACCAGAGAGCTCCA | |
| | GGGGAGAGAGCTAAAGCTTACTAACCTTCAGGAAAATTTGAGTGAAGTCAGT | |
| | CAAGTGAAAGAGACTTTGGAAAAAGAACTTCAGATTTTGAAAGAAAAGTTTG | |
| | CTGAAGCTTCAGAGGAGGCAGTCTCTGTTCAGAGAAGTATGCAAGAAACTGT | |
| | AAATAAGTTACACCAAAAGGAGGAACAGTTTAACATGCTGTCTTCTGACTTG | |
| | GAGAAGCTGAGAGAAAACTTAGCAGATATGGAGGCAAAATTTAGAGAGAAAG | |
| | ATGAGAGAGAAGAGCAGCTGATAAAGGCAAAGGAAAAACTGGAAAATGACAT | |
| | TGCAGAAATAATGAAGATGTCAGGAGATAACTCTTCTCAGCTGACAAAAATG | |
| | AACGATGAATTACGTCTGAAAGAAAGAGATGTAGAAGAATTACAGCTAAAAC | |
| | TTACAAAGGCTAATGAAAATGCAAGTTTTCTGCAAAAAAGTATTGAGGACAT | |
| | GACTGTCAAAGCTGAACAGAGCCAGCAAGAAGCAGCTAAAAAGCATGAGGAA | |
| | GAAAAGAAAGAATTGGAGAGGAAATTGTCGGACCTGGAAAAGAAAATGGAAA | |
| | CAAGCCACAACCAGTGTCAGGAGCTGAAAGCCAGGTATGAGAGAGCCACTTC | |
| | TGAGACAAAAACCAAGCATGAAGAAATCCTACAGAACCTCCAGAAGACGCTG | |
| | CTGGACACAGAGGACAAGCTGAAGGGCGCACGGGAGGAGAACAGTGGCTTGC | |
| | TGCAGGAGCTGGAGGAGCTGAGAAAGCAAGCCGACAAAGCCAAAGAGGATCC | |
| | AAAGTGGGAATTCCCTCGGAAGAACTTGGTTCTTGGAAAAACTCTAGGAGAA | |
| | GGCGAATTTGGAAAAGTGGTCAAGGCAACGGCCTTCCATCTGAAAGGCAGAG | |
| | CAGGGTACACCACGGTGGCCGTGAAGATGCTGAAAGAGAACGCCTCCCCGAG | |
| | TGAGCTGCGAGACCTGCTGTCAGAGTTCAACGTCCTGAAGCAGGTCAACCAC | |
| | CCACATGTCATCAAATTGTATGGGGCCTGCAGCCAGGATGGCCCGCTCCTCC | |
| | TCATCGTGGAGTACGCCAAATACGGCTCCCTGCGGGGCTTCCTCCGCGAGAG | |
| | CCGCAAAGTGGGGCCTGGCTACCTGGGCAGTGGAGGCAGCCGCAACTCCAGC | |
| | TCCCTGGACCACCCGGATGAGCGGGCCCTCACCATGGGCGACCTCATCTCAT | |
| | TTGCCTGGCAGATCTCACAGGGGATGCAGTATCTGGCCGAGATGAAGCTCGT | |
| | TCATCGGGACTTGGCAGCCAGAAACATCCTGGTAGCTGAGGGGCGGAAGATG | |
| | AAGATTTCGGATTTCGGCTTGTCCCGAGATGTTTATGAAGAGGATTCCTACG | |
| | TGAAGAGGAGCCAGGGTCGGATTCCAGTTAAATGGATGGCAATTGAATCCCT | |
| | TTTTGATCATATCTACACCACGCAAAGTGATGTATGGTCTTTTGGTGTCCTG | |
| | CTGTGGGAGATCGTGACCCTAGGGGGAAACCCCTATCCTGGGATTCCTCCTG | |
| | AGCGGCTCTTCAACCTTCTGAAGACCGGCCACCGGATGGAGAGGCCAGACAA | |
| | CTGCAGCGAGGAGATGTACCGCCTGATGCTGCAATGCTGGAAGCAGGAGCCG | |
| | GACAAAAGGCCGGTGTTTGCGGACATCAGCAAAGACCTGGAGAAGATGATGG | |
| | TTAAGAGGAGAGACTACTTGGACCTTGCGGCGTCCACTCCATCTGACTCCCT | |
| | GATTTATGACGACGGCCTCTCAGAGGAGGAGACACCGCTGGTGGACTGTAAT | |
| | AATGCCCCCCTCCCTCGAGCCCTCCCTTCCACATGGATTGAAAACAAACTCT | |
| | ATGGTAGAATTTCCCATGCATTTACTAGATTCTAG | |
| DLG5- | ATGGAGCCCCAGCGCCGGGAGCTGCTCGCCCAGTGTCAGCAGAGCCTGGCCC | 41 |
| RET | AGGCCATGACGGAGGTGGAAGCCGTGCTCGGGCTGCTCGAGGCCGCGGGAGC | |
| | GCTCAGTCCCGGCGAGCGGCGGCAGCTGGACGAGGAGGCGGGAGGCGCCAAG | |
| | GCGGAGCTGCTGCTCAAGCTGCTCTTGGCCAAGGAGCGGGACCACTTCCAGG | |
| | ACCTGCGGGCGGCGCTGGAGAAGACGCAGCCTCACCTGCTGCCCATTCTCTA | |
| | CCTGAACGGCGTCGTCGGGCCGCCGCAGCCCGCCGAAGGCGCGGGTTCTACC | |
| | TACAGCGTCCTGTCCACCATGCCCTCAGACTCAGAAAGCAGCAGCTCCCTCA | |
| | GCAGTGTGGGCACTACCGGGAAGGCGCCGTCCCCACCACCCCTCCTCACTGA | |
| | CCAGCAAGTGAATGAGAAGGTGGAGAACCTCTCCATTCAGCTGCGGCTGATG | |
| | ACCCGGGAGAGAAACGAGCTCCGCAAGCGCCTGGCCTTTGCTACGCATGGCA | |
| | CGGCCTTTGACAAGAGGCCCTACCACAGGCTGAATCCTGACTATGAGAGGCT | |
| | GAAGATCCAGTGCGTGCGAGCCATGTCGGACCTGCAGAGCCTGCAGAACCAG | |
| | CACACCAACGCCTTGAAGAGGTGTGAGGAGGTGGCCAAGGAGACTGACTTCT | |
| | ACCACACACTCCACAGCCGGCTCCTGAGTGACCAGACTCGGCTGAAGGATGA | |
| | CGTGGACATGCTGAGGCGGGAGAATGGGCAGCTGCTGCGGGAGCGAAACCTG | |
| | CTGCAGCAGTCATGGGAGGACATGAAGCGGCTCCACGAGGAGGACCAGAAGG | |
| | AGATCGGTGACCTCCGTGCCCAGCAGCAGCAGGTGTTGAAGCACAACGGGTC | |
| | ATCCGAGATTCTCAACAAACTGTATGACACGGCCATGGACAAGTTGGAGGTG | |
| | GTCAAGAAGGACTATGACGCCCTTCGGAAGAGGTACAGTGAGAAAGTCGCCA | |
| | TCCACAATGCAGACCTGAGCCGCCTGGAGCAGCTGGGGGAGGAGAACCAGCG | |
| | GTTGCTGAAGCAGACAGAGATGCTGACCCAGCAGAGGGACACGGCCATCCAG | |
| | CTGCAGCACCAGTGCGCCCTCTCCCTGAGGAGGTTTGAGGCGATCCACCATG | |
| | AGCTGAACAAGGCCACGGCGCAGAACAAGGACCTGCAGTGGGAGATGGAGCT | |
| | GCTGCAGTCAGAGCTGACCGAGCTGAGAACCACGCAGGTGAAGACAGCAAAG | |
| | GAGTCGGAGAAATACAGGGAGGAGCGGGACGCTGTGTACAGCGAGTACAAGC | |
| | TCATCATGAGTGAGCGTGACCAGGTCATCTCTGAGCTGGACAAGCTGCAGAC | |
| | CGAAGTGGAGCTGGCCGAGTCCAAGCTCAAGAGCAGCACATCTGAGAAGAAG | |
| | GCGGCCAATGAGGAGATGGAGGCGCTGCGGCAGATCAAAGACACGGTGACAA | |
| | TGGATGCTGGGAGAGCCAACAAGGAGGTTGAAATCCTTCGAAAGCAGTGCAA | |
| | GGCTCTGTGCCAGGAGCTGAAGGAAGCCCTCCAGGAGGCGGATGTGGCCAAG | |
| | TGCCGGCGGGACTGGGCCTTCCAGGAGCGAGACAAGATTGTAGCAGAGCGTG | |
| | ACAGCATCCGGACACTGTGTGACAACCTGAGGCGGGAGCGGGACCGTGCGGT | |
| | GAGCGAGCTGGCTGAGGCCCTGCGCAGCCTGGATGACACCCGCAAGCAGAAG | |
| | AATGATGTCAGCCGCGAGCTGAAGGAGCTCAAGGAACAGATGGAATCCCAGT | |
| | TGGAAAAGGAGGCCCGGTTCCGACAGCTGATGGCCCACAGCTCCCACGACTC | |
| | GGCCATTGACACGGATTCCATGGAGTGGGAAACGGAAGTTGTAGAGTTCGAG | |
| | AGGGAGACGGAGGATATTGACTTGAAGGCACTGGGGTTTGATATGGCAGAAG | |
| | GTGTGAATGAGCCTTGTTTCCCGGGGGACTGTGGCATATTTGTCACTAAAGT | |
| | GGACAAAGGAAGCATTGCTGATGGCCGCTTAAGGGTCAATGACTGGCTGCTG | |
| | AGAATCAACGATGTGGACCTCATCAACAAGGACAAGAAGCAGGCCATCAAGG | |
| | CGCTCCTCAATGGGGAGGGGGCCATCAACATGGTCGTGCGGCGGAGGAAGTC | |
| | CCTGGGTGGGAAGGTGGTCACGCCGCTGCACATCAACCTCAGTGGACAGAAA | |
| | GACAGTGGCATCAGTCTGGAGAATGGAGTGTATGCTGCCGCTGTGCTGCCTG | |
| | GAAGCCCTGCCGCTAAAGAAGGGTCCCTTGCTGTGGGAGACAGGATCGTTGC | |
| | GATCAATGGCATTGCACTGGACAACAAGTCTCTGAATGAATGTGAATCTCTG | |
| | CTGCGCAGCTGCCAGGACTCCCTGACCCTGTCCCTCCTGAAGGAGGATCCAA | |
| | AGTGGGAATTCCCTCGGAAGAACTTGGTTCTTGGAAAAACTCTAGGAGAAGG | |
| | CGAATTTGGAAAAGTGGTCAAGGCAACGGCCTTCCATCTGAAAGGCAGAGCA | |
| | GGGTACACCACGGTGGCCGTGAAGATGCTGAAAGAGAACGCCTCCCCGAGTG | |
| | AGCTGCGAGACCTGCTGTCAGAGTTCAACGTCCTGAAGCAGGTCAACCACCC | |
| | ACATGTCATCAAATTGTATGGGGCCTGCAGCCAGGATGGCCCGCTCCTCCTC | |
| | ATCGTGGAGTACGCCAAATACGGCTCCCTGCGGGGCTTCCTCCGCGAGAGCC | |
| | GCAAAGTGGGGCCTGGCTACCTGGGCAGTGGAGGCAGCCGCAACTCCAGCTC | |
| | CCTGGACCACCCGGATGAGCGGGCCCTCACCATGGGCGACCTCATCTCATTT | |
| | GCCTGGCAGATCTCACAGGGGATGCAGTATCTGGCCGAGATGAAGCTCGTTC | |
| | ATCGGGACTTGGCAGCCAGAAACATCCTGGTAGCTGAGGGGCGGAAGATGAA | |
| | GATTTCGGATTTCGGCTTGTCCCGAGATGTTTATGAAGAGGATTCCTACGTG | |
| | AAGAGGAGCCAGGGTCGGATTCCAGTTAAATGGATGGCAATTGAATCCCTTT | |
| | TTGATCATATCTACACCACGCAAAGTGATGTATGGTCTTTTGGTGTCCTGCT | |
| | GTGGGAGATCGTGACCCTAGGGGGAAACCCCTATCCTGGGATTCCTCCTGAG | |
| | CGGCTCTTCAACCTTCTGAAGACCGGCCACCGGATGGAGAGGCCAGACAACT | |
| | GCAGCGAGGAGATGTACCGCCTGATGCTGCAATGCTGGAAGCAGGAGCCGGA | |
| | CAAAAGGCCGGTGTTTGCGGACATCAGCAAAGACCTGGAGAAGATGATGGTT | |
| | AAGAGGAGAGACTACTTGGACCTTGCGGCGTCCACTCCATCTGACTCCCTGA | |
| | TTTATGACGACGGCCTCTCAGAGGAGGAGACACCGCTGGTGGACTGTAATAA | |
| | TGCCCCCCTCCCTCGAGCCCTCCCTTCCACATGGATTGAAAACAAACTCTAT | |
| | GGTAGAATTTCCCATGCATTTACTAGATTCTAG | |
| DLG5- | ATGGAGCCCCAGCGCCGGGAGCTGCTCGCCCAGTGTCAGCAGAGCCTGGCCC | 42 |
| RET | AGGCCATGACGGAGGTGGAAGCCGTGCTCGGGCTGCTCGAGGCCGCGGGAGC | |
| | GCTCAGTCCCGGCGAGCGGCGGCAGCTGGACGAGGAGGCGGGAGGCGCCAAG | |
| | GCGGAGCTGCTGCTCAAGCTGCTCTTGGCCAAGGAGCGGGACCACTTCCAGG | |
| | ACCTGCGGGCGGCGCTGGAGAAGACGCAGCCTCACCTGCTGCCCATTCTCTA | |
| | CCTGAACGGCGTCGTCGGGCCGCCGCAGCCCGCCGAAGGCGCGGGTTCTACC | |
| | TACAGCGTCCTGTCCACCATGCCCTCAGACTCAGAAAGCAGCAGCTCCCTCA | |
| | GCAGTGTGGGCACTACCGGGAAGGCGCCGTCCCCACCACCCCTCCTCACTGA | |
| | CCAGCAAGTGAATGAGAAGGTGGAGAACCTCTCCATTCAGCTGCGGCTGATG | |
| | ACCCGGGAGAGAAACGAGCTCCGCAAGCGCCTGGCCTTTGCTACGCATGGCA | |
| | CGGCCTTTGACAAGAGGCCCTACCACAGGCTGAATCCTGACTATGAGAGGCT | |
| | GAAGATCCAGTGCGTGCGAGCCATGTCGGACCTGCAGAGCCTGCAGAACCAG | |
| | CACACCAACGCCTTGAAGAGGTGTGAGGAGGTGGCCAAGGAGACTGACTTCT | |
| | ACCACACACTCCACAGCCGGCTCCTGAGTGACCAGACTCGGCTGAAGGATGA | |
| | CGTGGACATGCTGAGGCGGGAGAATGGGCAGCTGCTGCGGGAGCGAAACCTG | |
| | CTGCAGCAGTCATGGGAGGACATGAAGCGGCTCCACGAGGAGGACCAGAAGG | |
| | AGATCGGTGACCTCCGTGCCCAGCAGCAGCAGGTGTTGAAGCACAACGGGTC | |
| | ATCCGAGATTCTCAACAAACTGTATGACACGGCCATGGACAAGTTGGAGGTG | |
| | GTCAAGAAGGACTATGACGCCCTTCGGAAGAGGTACAGTGAGAAAGTCGCCA | |
| | TCCACAATGCAGACCTGAGCCGCCTGGAGCAGCTGGGGGAGGAGAACCAGCG | |
| | GTTGCTGAAGCAGACAGAGATGCTGACCCAGCAGAGGGACACGGCCATCCAG | |
| | CTGCAGCACCAGTGCGCCCTCTCCCTGAGGAGGTTTGAGGCGATCCACCATG | |
| | AGCTGAACAAGGCCACGGCGCAGAACAAGGACCTGCAGTGGGAGATGGAGCT | |
| | GCTGCAGTCAGAGCTGACCGAGCTGAGAACCACGCAGGTGAAGACAGCAAAG | |
| | GAGTCGGAGAAATACAGGGAGGAGCGGGACGCTGTGTACAGCGAGTACAAGC | |
| | TCATCATGAGTGAGCGTGACCAGGTCATCTCTGAGCTGGACAAGCTGCAGAC | |
| | CGAAGTGGAGCTGGCCGAGTCCAAGCTCAAGAGCAGCACATCTGAGAAGAAG | |
| | GCGGCCAATGAGGAGATGGAGGCGCTGCGGCAGATCAAAGACACGGTGACAA | |
| | TGGATGCTGGGAGAGCCAACAAGGAGGTTGAAATCCTTCGAAAGCAGTGCAA | |
| | GGCTCTGTGCCAGGAGCTGAAGGAAGCCCTCCAGGAGGCGGATGTGGCCAAG | |
| | TGCCGGCGGGACTGGGCCTTCCAGGAGCGAGACAAGATTGTAGCAGAGCGTG | |
| | ACAGCATCCGGACACTGTGTGACAACCTGAGGCGGGAGCGGGACCGTGCGGT | |
| | GAGCGAGCTGGCTGAGGCCCTGCGCAGCCTGGATGACACCCGCAAGCAGAAG | |
| | AATGATGTCAGCCGCGAGCTGAAGGAGCTCAAGGAACAGATGGAATCCCAGT | |
| | TGGAAAAGGAGGCCCGGTTCCGACAGCTGATGGCCCACAGCTCCCACGACTC | |
| | GGCCATTGACACGGATTCCATGGAGTGGGAAACGGAAGTTGTAGAGTTCGAG | |
| | AGGGAGACGGAGGATATTGACTTGAAGGCACTGGGGTTTGATATGGCAGAAG | |
| | GTGTGAATGAGCCTTGTTTCCCGGGGGACTGTGGCATATTTGTCACTAAAGT | |
| | GGACAAAGGAAGCATTGCTGATGGCCGCTTAAGGGTCAATGACTGGCTGCTG | |
| | AGAATCAACGATGTGGACCTCATCAACAAGGACAAGAAGCAGGCCATCAAGG | |
| | CGCTCCTCAATGGGGAGGGGGCCATCAACATGGTCGTGCGGCGGAGGAAGTC | |
| | CCTGGGTGGGAAGGTGGTCACGCCGCTGCACATCAACCTCAGTGGACAGAAA | |
| | GACAGTGGCATCAGTCTGGAGAATGGAGTGTATGCTGCCGCTGTGCTGCCTG | |
| | GAAGCCCTGCCGCTAAAGAAGGGTCCCTTGCTGTGGGAGACAGGATCGTTGC | |
| | GATCAATGGCATTGCACTGGACAACAAGTCTCTGAATGAATGTGAATCTCTG | |
| | CTGCGCAGCTGCCAGGACTCCCTGACCCTGTCCCTCCTGAAGGTATTCCCTC | |
| | AGAGCTCCTCGTGGAGTGGCCAGAACATTTTTGAAAATATCAAAGACTCTGA | |
| | TAAGATGCTGAGTTTTCGAGCCCATGGCCCGGAGGTCCAGGCTCATAACAAA | |
| | CGGAACTTGATACAGCACAATAACTCCACGCAGACAGACATCTTCTACACGG | |
| | ACAGGCTGGAAGACAGGAAGGAGCCAGGCCCCCCAGGAGGCAGCAGCTCCTT | |
| | TCTGCATAAGCCATTCCCTGGGGGACCCTTGCAGGTCTGCCCCCAGGCCTGT | |
| | CCCAGTGCCTCTGAGCGTAGCCTGAGCTCCTTCCGCTCAGATGCCTCTGGGG | |
| | ACCGTGGCTTTGGGCTGGTGGACGTGCGTGGCCGGCGGCCACTGCTGCCCTT | |
| | TGAGACCGAGGTGGGCCCCTGTGGGGTTGGGGAGGCCTCCCTGGACAAGGCA | |
| | GACTCTGAAGGCTCCAACAGCGGCGGGACCTGGCCCAAGGCCATGCTCAGCT | |
| | CCACGGCAGTGCCTGAGAAGCTCTCTGTTTATAAAAAGCCAAAGCAAAGAAA | |
| | GTCCATCTTTGACCCTAACACTTTCAAACGCCCCCAGACACCCCCCAAAATA | |
| | GACTACCTGCTTCCAGGTCCTGGGCCTGCTCACTCTCCCCAGCCCTCCAAGA | |
| | GGGCGGGGCCTCTGACACCCCCAAAACCTCCCAGAAGGAGCGACTCCATTAA | |
| | GTTCCAGCACAGGCTGGAGACTAGCTCCGAGTCAGAAGCCACTCTGGTGGGC | |
| | AGCTCCCCATCCACTAGTCCCCCGAGCGCCCTGCCCCCTGACGTGGACCCCG | |
| | GGGAGCCCATGCACGCATCACCCCCTCGCAAGGCCAGGGTCCGCATTGCTTC | |
| | CAGCTACTACCCTGAAGGAGATGGGGACTCCTCCCACCTGCCGGCCAAGAAA | |
| | TCCTGTGATGAGGACCTCACCTCCCAGAAGGTGGATGAGCTGGGGCAGAAGC | |
| | GTCGCCGGCCAAAATCTGCTCCCAGTTTTCGGCCGAAGCTTGCTCCAGTAGT | |
| | GATTCCTGCTCAGTTCCTGGAGGAACAGAAGTGTGTCCCGGCCAGTGGAGAA | |
| | CTCTCCCCGGAGCTCCAGGAGTGGGCACCTTACTCGCCTGGGCATTCCAGCC | |
| | GGCACAGCAACCCCCCGCTATACCCTAGCAGGCCGTCTGTGGGCACTGTTCC | |
| | CCGGAGTTTGACCCCCAGCACCACTGTGAGCTCCATCCTGCGGAACCCCATC | |
| | TACACTGTGCGCAGTCACAGGGTCGGCCCCTGCAGCTCTCCACCTGCGGCCC | |
| | GAGATGCTGGCCCCCAGGGTTTGCATCCCAGTGTCCAGCACCAGGGACGCCT | |
| | GAGCCTGGACCTGAGCCACAGGACCTGCAGCGACTACTCCGAGATGAGAGCC | |
| | ACCCATGGGTCCAACTCACTGCCCTCCAGCGCCCGCCTGGGTTCTTCGAGTA | |
| | ACTTGCAGTTCAAGGCGGAACGCATTAAAATCCCATCAACACCAAGATATCC | |
| | GCGGAGTGTCGTGGGCTCCGAGAGAGGTTCAGTGTCACATTCTGAATGCAGC | |
| | ACTCCTCCACAGTCACCCCTGAACATCGACACCCTGTCCTCTTGTAGCCAGT | |
| | CCCAGACCTCAGCCTCCACATTGCCCAGAATCGCTGTCAACCCCGCGTCCCT | |
| | CGGGGAGCGGAGAAAGGACAGGCCTTATGTGGAGGAGCCACGCCACGTGAAG | |
| | GTGCAGAAGGGCTCAGAGCCGCTGGGCATCTCCATCGTGAGTGGAGAGAAGG | |
| | GCGGCATCTACGTCTCCAAGGTGACCGTGGGGAGCATCGCTCACCAGGCTGG | |
| | CCTCGAGTATGGGGATCAGTTACTGGAGTTCAACGGCATAAACCTGCGGAGC | |
| | GCCACGGAGCAGCAGGCGCGGCTCATCATCGGGCAGCAGTGTGATACCATCA | |
| | CCATCCTGGCCCAGTACAACCCCCACGTGCACCAGCTCAGCAGCCACTCCCG | |
| | GTCCAGCTCACACCTGGACCCTGCCGGTACCCACTCCACTCTCCAGGGCAGT | |
| | GGCACCACCACCCCGGAGCATCCATCTGTCATCGACCCACTGATGGAGCAGG | |
| | ACGAGGGGCCTAGCACCCCCCCAGCCAAGCAGAGCAGCTCCAGGATTGCGGG | |
| | AGATGCCAACAAGAAGACCCTGGAGCCACGCGTTGTCTTCATCAAAAAGTCC | |
| | CAGCTGGAGCTTGGGGTGCACTTGTGTGGTGGGAACCTGCATGGGGTGTTTG | |
| | TGGCCGAGGTGGAGGATGACAGTCCTGCCAAGGGTCCTGACGGCCTCGTGCC | |
| | AGGGGACCTCATCCTGGAGTATGGCAGCCTGGACGTGCGGAACAAGACAGTG | |
| | GAGGAAGTCTATGTGGAGATGCTGAAGCCCAGGGATGGCGTCCGCCTGAAGG | |
| | TGCAGTACCGCCCTGAGGAGTTCACGAAGGCCAAGGGCCTGCCTGGTGACAG | |
| | CTTCTACATCAGGGCCCTGTACGACCGGCTGGCAGATGTGGAGCAAGAGTTG | |
| | AGCTTTAAGAAGGACGACATCCTCTACGTGGATGACACCTTACCCCAGGGCA | |
| | CGTTCGGGTCCTGGATGGCTTGGCAGCTGGACGAGAATGCCCAGAAGATCCA | |
| | GCGCGGGCAGATTCCCAGCAAATATGTGATGGACCAAGAATTCTCCAGGAGG | |
| | CTCAGCATGTCTGAAGTCAAAGATGACAATAGCGCCACAAAGACGCTGTCAG | |
| | CGGCTGCACGCCGGTCCTTTTTTCGGAGGAAACACAAGCACAAACGCAGCGG | |
| | GTCCAAGGACGGGAAAGACCTGCTCGCCTTGGATGCCTTTTCCAGTGACTCC | |
| | ATTCCACTCTTTGAAGATGTGGCCGAGGAGGCGGGCTGCCCCCTGTCCTGTG | |
| | CAGTCAGCAAGAGACGGCTGGAGTGTGAGGAGTGTGGCGGCCTGGGCTCCCC | |
| | AACAGGCAGGTGTGAGTGGAGGCAAGGAGATGGCAAAGGGATCACCAGGAAC | |
| | TTCTCCACCTGCTCTCCCAGCACCAAGACCTGCCCCGACGGCCACTGCGATG | |
| | TTGTGGAGACCCAAGACATCAACATTTGCCCTCAGGACTGCCTCCGGGGCAG | |
| | CATTGTTGGGGGACACGAGCCTGGGGAGCCCCGGGGGATTAAAGCTGGCTAT | |
| | GGCACCTGCAACTGCTTCCCTGAGGAGGAGAAGTGCTTCTGCGAGCCCGAAG | |
| | ACATCCAGGATCCACTGTGCGACGAGCTGTGCCGCACGGTGATCGCAGCCGC | |
| | TGTCCTCTTCTCCTTCATCGTCTCGGTGCTGCTGTCTGCCTTCTGCATCCAC | |
| | TGCTACCACAAGTTTGCCCACAAGCCACCCATCTCCTCAGCTGAGATGACCT | |
| | TCCGGAGGCCCGCCCAGGCCTTCCCGGTCAGCTACTCCTCTTCCGGTGCCCG | |
| | CCGGCCCTCGCTGGACTCCATGGAGAACCAGGTCTCCGTGGATGCCTTCAAG | |
| | ATCCTGGAGGATCCAAAGTGGGAATTCCCTCGGAAGAACTTGGTTCTTGGAA | |
| | AAACTCTAGGAGAAGGCGAATTTGGAAAAGTGGTCAAGGCAACGGCCTTCCA | |
| | TCTGAAAGGCAGAGCAGGGTACACCACGGTGGCCGTGAAGATGCTGAAAGAG | |
| | AACGCCTCCCCGAGTGAGCTGCGAGACCTGCTGTCAGAGTTCAACGTCCTGA | |
| | AGCAGGTCAACCACCCACATGTCATCAAATTGTATGGGGCCTGCAGCCAGGA | |
| | TGGCCCGCTCCTCCTCATCGTGGAGTACGCCAAATACGGCTCCCTGCGGGGC | |
| | TTCCTCCGCGAGAGCCGCAAAGTGGGGCCTGGCTACCTGGGCAGTGGAGGCA | |
| | GCCGCAACTCCAGCTCCCTGGACCACCCGGATGAGCGGGCCCTCACCATGGG | |
| | CGACCTCATCTCATTTGCCTGGCAGATCTCACAGGGGATGCAGTATCTGGCC | |
| | GAGATGAAGCTCGTTCATCGGGACTTGGCAGCCAGAAACATCCTGGTAGCTG | |
| | AGGGGCGGAAGATGAAGATTTCGGATTTCGGCTTGTCCCGAGATGTTTATGA | |
| | AGAGGATTCCTACGTGAAGAGGAGCCAGGGTCGGATTCCAGTTAAATGGATG | |
| | GCAATTGAATCCCTTTTTGATCATATCTACACCACGCAAAGTGATGTATGGT | |
| | CTTTTGGTGTCCTGCTGTGGGAGATCGTGACCCTAGGGGGAAACCCCTATCC | |
| | TGGGATTCCTCCTGAGCGGCTCTTCAACCTTCTGAAGACCGGCCACCGGATG | |
| | GAGAGGCCAGACAACTGCAGCGAGGAGATGTACCGCCTGATGCTGCAATGCT | |
| | GGAAGCAGGAGCCGGACAAAAGGCCGGTGTTTGCGGACATCAGCAAAGACCT | |
| | GGAGAAGATGATGGTTAAGAGGAGAGACTACTTGGACCTTGCGGCGTCCACT | |
| | CCATCTGACTCCCTGATTTATGACGACGGCCTCTCAGAGGAGGAGACACCGC | |
| | TGGTGGACTGTAATAATGCCCCCCTCCCTCGAGCCCTCCCTTCCACATGGAT | |
| | TGAAAACAAACTCTATGGTAGAATTTCCCATGCATTTACTAGATTCTAG | |
| ERC1- | ATGTATGGAAGTGCCCGCTCTGTTGGGAAGGTGGAGCCGAGCAGCCAGAGCC | 43 |
| RET | CTGGGCGTTCACCCAGGCTTCCACGTTCCCCTCGCTTGGGTCACCGTCGAAC | |
| | CAACAGTACGGGAGGGAGTTCGGGAAGCAGTGTTGGAGGTGGCAGTGGGAAA | |
| | ACCCTTTCAATGGAAAATATACAATCTTTAAATGCTGCCTATGCCACCTCTG | |
| | GCCCTATGTATCTAAGTGACCATGAAAATGTGGGTTCAGAAACACCTAAAAG | |
| | CACCATGACACTTGGCCGTTCTGGGGGACGTCTGCCTTACGGTGTTCGGATG | |
| | ACTGCTATGGGTAGTAGCCCCAATATAGCTAGCAGTGGGGTTGCTAGTGACA | |
| | CCATAGCATTTGGAGAGCATCACCTCCCTCCTGTGAGTATGGCATCCACTGT | |
| | ACCTCACTCCCTTCGTCAGGCGAGAGATAACACAATCATGGATCTGCAGACA | |
| | CAGCTGAAGGAAGTATTAAGAGAAAATGATCTCTTGCGGAAGGATGTGGAAG | |
| | TAAAGGAGAGCAAATTGAGTTCTTCAATGAATAGCATCAAGACCTTCTGGAG | |
| | CCCAGAGCTGAAGAAGGAACGAGCCCTGAGAAAAGATGAAGCTTCCAAAATC | |
| | ACCATTTGGAAGGAACAGTACAGAGTTGTACAGGAGGAAAACCAGCACATGC | |
| | AGATGACAATCCAGGCTCTCCAGGATGAATTGCGGATCCAGAGGGACCTGAA | |
| | TCAGCTGTTTCAGCAGGATAGTAGCAGCAGGACTGGCGAACCTTGTGTAGCA | |
| | GAGCTGACAGAGGAGAACTTTCAGAGGCTTCATGCTGAGCATGAGCGGCAGG | |
| | CCAAAGAGCTGTTTCTTCTTCGAAAGACATTGGAGGAAATGGAGCTGCGTAT | |
| | TGAGACTCAAAAGCAGACCCTAAATGCTCGGGATGAATCCATTAAGAAGCTT | |
| | CTGGAAATGTTGCAGAGCAAAGGACTTTCTGCCAAGGCTACCGAGGAAGACC | |
| | ATGAGAGAACAAGACGACTGGCAGAGGCAGAGATGCACGTTCATCACCTAGA | |
| | AAGCCTTTTGGAGCAGAAGGAAAAAGAGAACAGTATGTTGAGAGAGGAGATG | |
| | CATCGAAGGTTTGAGAATGCTCCTGATTCTGCCAAAACAAAAGCTCTGCAAA | |
| | CTGTTATTGAGATGAAGGATTCAAAAATTTCCTCTATGGAGCGTGGGCTTCG | |
| | AGACCTGGAAGAGGAAATTCAGATGCTGAAATCGAATGGTGCTTTGAGTACT | |
| | GAGGAAAGGGAAGAAGAAATGAAGCAAATGGAAGTGTATCGGAGCCATTCTA | |
| | AATTTATGAAAAATAAGATTGGCCAGGTGAAACAGGAGCTGTCCAGAAAGGA | |
| | CACAGAACTACTCGCCCTGCAGACAAAGCTAGAAACACTCACAAACCAGTTC | |
| | TCAGATAGTAAACAGCACATTGAAGTGTTGAAGGAGTCCTTGACTGCTAAGG | |
| | AGCAGAGGGCTGCCATCCTGCAGACTGAGGTGGATGCTCTCCGATTGCGTTT | |
| | GGAAGAGAAGGAAACCATGTTGAATAAAAAGACAAAACAAATTCAGGATATG | |
| | GCTGAAGAGAAGGGGACACAAGCTGGAGAGATACATGACCTCAAGGACATGT | |
| | TGGATGTGAAGGAGCGGAAGGTTAATGTTCTTCAGAAGAAGATTGAAAATCT | |
| | TCAAGAGCAGCTTAGAGACAAGGAAAAGCAGATGAGCAGCTTGAAAGAACGG | |
| | GTCAAATCCTTGCAGGCTGACACCACCAACACTGACACTGCCTTGACAACTT | |
| | TGGAGGAGGCCCTTGCAGAGAAAGAGCGGACAATTGAACGCTTAAAGGAGCA | |
| | GAGGGACAGAGATGAGCGAGAGAAGCAAGAGGAAATTGATAACTACAAAAAA | |
| | GATCTTAAAGACTTGAAGGAAAAAGTCAGCCTGTTGCAAGGCGACCTTTCAG | |
| | AGAAAGAGGCTTCACTTTTGGATCTGAAAGAGCATGCTTCTTCTCTGGCATC | |
| | CTCAGGACTGAAAAAGGACTCACGGCTTAAGACACTAGAGATTGCTTTGGAG | |
| | CAGAAGAAGGAGGAGTGTCTGAAAATGGAATCACAATTGAAAAAGGCACATG | |
| | AGGCAGCATTGGAAGCCAGAGCCAGTCCAGAGATGAGTGACCGAATACAGCA | |
| | CTTGGAGAGAGAGATCACCAGGTACAAAGATGAATCTAGCAAGGCCCAGGCA | |
| | GAAGTTGATCGACTCTTAGAAATCTTGAAGGAGGTGGAAAATGAGAAGAATG | |
| | ACAAAGATAAGAAGATAGCTGAGTTGGAAAGGCAAGTGAAAGACCAGAATAA | |
| | GAAGGTAGCAAATCTGAAGCACAAGGAACAGGTGGAAAAAAAGAAGAGTGCA | |
| | CAAATGTTAGAGGAGGCGCGACGACGGGAGGACAATCTCAACGACAGCTCTC | |
| | AGCAGCTACAGGAGGATCCAAAGTGGGAATTCCCTCGGAAGAACTTGGTTCT | |
| | TGGAAAAACTCTAGGAGAAGGCGAATTTGGAAAAGTGGTCAAGGCAACGGCC | |
| | TTCCATCTGAAAGGCAGAGCAGGGTACACCACGGTGGCCGTGAAGATGCTGA | |
| | AAGAGAACGCCTCCCCGAGTGAGCTGCGAGACCTGCTGTCAGAGTTCAACGT | |
| | CCTGAAGCAGGTCAACCACCCACATGTCATCAAATTGTATGGGGCCTGCAGC | |
| | CAGGATGGCCCGCTCCTCCTCATCGTGGAGTACGCCAAATACGGCTCCCTGC | |
| | GGGGCTTCCTCCGCGAGAGCCGCAAAGTGGGGCCTGGCTACCTGGGCAGTGG | |
| | AGGCAGCCGCAACTCCAGCTCCCTGGACCACCCGGATGAGCGGGCCCTCACC | |
| | ATGGGCGACCTCATCTCATTTGCCTGGCAGATCTCACAGGGGATGCAGTATC | |
| | TGGCCGAGATGAAGCTCGTTCATCGGGACTTGGCAGCCAGAAACATCCTGGT | |
| | AGCTGAGGGGCGGAAGATGAAGATTTCGGATTTCGGCTTGTCCCGAGATGTT | |
| | TATGAAGAGGATTCCTACGTGAAGAGGAGCCAGGGTCGGATTCCAGTTAAAT | |
| | GGATGGCAATTGAATCCCTTTTTGATCATATCTACACCACGCAAAGTGATGT | |
| | ATGGTCTTTTGGTGTCCTGCTGTGGGAGATCGTGACCCTAGGGGGAAACCCC | |
| | TATCCTGGGATTCCTCCTGAGCGGCTCTTCAACCTTCTGAAGACCGGCCACC | |
| | GGATGGAGAGGCCAGACAACTGCAGCGAGGAGATGTACCGCCTGATGCTGCA | |
| | ATGCTGGAAGCAGGAGCCGGACAAAAGGCCGGTGTTTGCGGACATCAGCAAA | |
| | GACCTGGAGAAGATGATGGTTAAGAGGAGAGACTACTTGGACCTTGCGGCGT | |
| | CCACTCCATCTGACTCCCTGATTTATGACGACGGCCTCTCAGAGGAGGAGAC | |
| | ACCGCTGGTGGACTGTAATAATGCCCCCCTCCCTCGAGCCCTCCCTTCCACA | |
| | TGGATTGAAAACAAACTCTATGGTAGAATTTCCCATGCATTTACTAGATTCT | |
| | AG | |
| ERC1- | ATGTATGGAAGTGCCCGCTCTGTTGGGAAGGTGGAGCCGAGCAGCCAGAGCC | 44 |
| RET | CTGGGCGTTCACCCAGGCTTCCACGTTCCCCTCGCTTGGGTCACCGTCGAAC | |
| | CAACAGTACGGGAGGGAGTTCGGGAAGCAGTGTTGGAGGTGGCAGTGGGAAA | |
| | ACCCTTTCAATGGAAAATATACAATCTTTAAATGCTGCCTATGCCACCTCTG | |
| | GCCCTATGTATCTAAGTGACCATGAAAATGTGGGTTCAGAAACACCTAAAAG | |
| | CACCATGACACTTGGCCGTTCTGGGGGACGTCTGCCTTACGGTGTTCGGATG | |
| | ACTGCTATGGGTAGTAGCCCCAATATAGCTAGCAGTGGGGTTGCTAGTGACA | |
| | CCATAGCATTTGGAGAGCATCACCTCCCTCCTGTGAGTATGGCATCCACTGT | |
| | ACCTCACTCCCTTCGTCAGGCGAGAGATAACACAATCATGGATCTGCAGACA | |
| | CAGCTGAAGGAAGTATTAAGAGAAAATGATCTCTTGCGGAAGGATGTGGAAG | |
| | TAAAGGAGAGCAAATTGAGTTCTTCAATGAATAGCATCAAGACCTTCTGGAG | |
| | CCCAGAGCTGAAGAAGGAACGAGCCCTGAGAAAAGATGAAGCTTCCAAAATC | |
| | ACCATTTGGAAGGAACAGTACAGAGTTGTACAGGAGGAAAACCAGCACATGC | |
| | AGATGACAATCCAGGCTCTCCAGGATGAATTGCGGATCCAGAGGGACCTGAA | |
| | TCAGCTGTTTCAGCAGGATAGTAGCAGCAGGACTGGCGAACCTTGTGTAGCA | |
| | GAGCTGACAGAGGAGAACTTTCAGAGGCTTCATGCTGAGCATGAGCGGCAGG | |
| | CCAAAGAGCTGTTTCTTCTTCGAAAGACATTGGAGGAAATGGAGCTGCGTAT | |
| | TGAGACTCAAAAGCAGACCCTAAATGCTCGGGATGAATCCATTAAGAAGCTT | |
| | CTGGAAATGTTGCAGAGCAAAGGACTTTCTGCCAAGGCTACCGAGGAAGACC | |
| | ATGAGAGAACAAGACGACTGGCAGAGGCAGAGATGCACGTTCATCACCTAGA | |
| | AAGCCTTTTGGAGCAGAAGGAAAAAGAGAACAGTATGTTGAGAGAGGAGATG | |
| | CATCGAAGGTTTGAGAATGCTCCTGATTCTGCCAAAACAAAAGCTCTGCAAA | |
| | CTGTTATTGAGATGAAGGATTCAAAAATTTCCTCTATGGAGCGTGGGCTTCG | |
| | AGACCTGGAAGAGGAAATTCAGATGCTGAAATCGAATGGTGCTTTGAGTACT | |
| | GAGGAAAGGGAAGAAGAAATGAAGCAAATGGAAGTGTATCGGAGCCATTCTA | |
| | AATTTATGAAAAATAAGATTGGCCAGGTGAAACAGGAGCTGTCCAGAAAGGA | |
| | CACAGAACTACTCGCCCTGCAGACAAAGCTAGAAACACTCACAAACCAGTTC | |
| | TCAGATAGTAAACAGCACATTGAAGTGTTGAAGGAGTCCTTGACTGCTAAGG | |
| | AGCAGAGGGCTGCCATCCTGCAGACTGAGGTGGATGCTCTCCGATTGCGTTT | |
| | GGAAGAGAAGGAAACCATGTTGAATAAAAAGACAAAACAAATTCAGGATATG | |
| | GCTGAAGAGAAGGGGACACAAGCTGGAGAGATACATGACCTCAAGGACATGT | |
| | TGGATGTGAAGGAGCGGAAGGTTAATGTTCTTCAGAAGAAGATTGAAAATCT | |
| | TCAAGAGCAGCTTAGAGACAAGGAAAAGCAGATGAGCAGCTTGAAAGAACGG | |
| | GTCAAATCCTTGCAGGCTGACACCACCAACACTGACACTGCCTTGACAACTT | |
| | TGGAGGAGGCCCTTGCAGAGAAAGAGGATCCAAAGTGGGAATTCCCTCGGAA | |
| | GAACTTGGTTCTTGGAAAAACTCTAGGAGAAGGCGAATTTGGAAAAGTGGTC | |
| | AAGGCAACGGCCTTCCATCTGAAAGGCAGAGCAGGGTACACCACGGTGGCCG | |
| | TGAAGATGCTGAAAGAGAACGCCTCCCCGAGTGAGCTGCGAGACCTGCTGTC | |
| | AGAGTTCAACGTCCTGAAGCAGGTCAACCACCCACATGTCATCAAATTGTAT | |
| | GGGGCCTGCAGCCAGGATGGCCCGCTCCTCCTCATCGTGGAGTACGCCAAAT | |
| | ACGGCTCCCTGCGGGGCTTCCTCCGCGAGAGCCGCAAAGTGGGGCCTGGCTA | |
| | CCTGGGCAGTGGAGGCAGCCGCAACTCCAGCTCCCTGGACCACCCGGATGAG | |
| | CGGGCCCTCACCATGGGCGACCTCATCTCATTTGCCTGGCAGATCTCACAGG | |
| | GGATGCAGTATCTGGCCGAGATGAAGCTCGTTCATCGGGACTTGGCAGCCAG | |
| | AAACATCCTGGTAGCTGAGGGGCGGAAGATGAAGATTTCGGATTTCGGCTTG | |
| | TCCCGAGATGTTTATGAAGAGGATTCCTACGTGAAGAGGAGCCAGGGTCGGA | |
| | TTCCAGTTAAATGGATGGCAATTGAATCCCTTTTTGATCATATCTACACCAC | |
| | GCAAAGTGATGTATGGTCTTTTGGTGTCCTGCTGTGGGAGATCGTGACCCTA | |
| | GGGGGAAACCCCTATCCTGGGATTCCTCCTGAGCGGCTCTTCAACCTTCTGA | |
| | AGACCGGCCACCGGATGGAGAGGCCAGACAACTGCAGCGAGGAGATGTACCG | |
| | CCTGATGCTGCAATGCTGGAAGCAGGAGCCGGACAAAAGGCCGGTGTTTGCG | |
| | GACATCAGCAAAGACCTGGAGAAGATGATGGTTAAGAGGAGAGACTACTTGG | |
| | ACCTTGCGGCGTCCACTCCATCTGACTCCCTGATTTATGACGACGGCCTCTC | |
| | AGAGGAGGAGACACCGCTGGTGGACTGTAATAATGCCCCCCTCCCTCGAGCC | |
| | CTCCCTTCCACATGGATTGAAAACAAACTCTATGGTAGAATTTCCCATGCAT | |
| | TTACTAGATTCTAG | |
| ERC1- | ATGTATGGAAGTGCCCGCTCTGTTGGGAAGGTGGAGCCGAGCAGCCAGAGCC | 45 |
| RET | CTGGGCGTTCACCCAGGCTTCCACGTTCCCCTCGCTTGGGTCACCGTCGAAC | |
| | CAACAGTACGGGAGGGAGTTCGGGAAGCAGTGTTGGAGGTGGCAGTGGGAAA | |
| | ACCCTTTCAATGGAAAATATACAATCTTTAAATGCTGCCTATGCCACCTCTG | |
| | GCCCTATGTATCTAAGTGACCATGAAAATGTGGGTTCAGAAACACCTAAAAG | |
| | CACCATGACACTTGGCCGTTCTGGGGGACGTCTGCCTTACGGTGTTCGGATG | |
| | ACTGCTATGGGTAGTAGCCCCAATATAGCTAGCAGTGGGGTTGCTAGTGACA | |
| | CCATAGCATTTGGAGAGCATCACCTCCCTCCTGTGAGTATGGCATCCACTGT | |
| | ACCTCACTCCCTTCGTCAGGCGAGAGATAACACAATCATGGATCTGCAGACA | |
| | CAGCTGAAGGAAGTATTAAGAGAAAATGATCTCTTGCGGAAGGATGTGGAAG | |
| | TAAAGGAGAGCAAATTGAGTTCTTCAATGAATAGCATCAAGACCTTCTGGAG | |
| | CCCAGAGCTGAAGAAGGAACGAGCCCTGAGAAAAGATGAAGCTTCCAAAATC | |
| | ACCATTTGGAAGGAACAGTACAGAGTTGTACAGGAGGAAAACCAGCACATGC | |
| | AGATGACAATCCAGGCTCTCCAGGATGAATTGCGGATCCAGAGGGACCTGAA | |
| | TCAGCTGTTTCAGCAGGATAGTAGCAGCAGGACTGGCGAACCTTGTGTAGCA | |
| | GAGCTGACAGAGGAGAACTTTCAGAGGCTTCATGCTGAGCATGAGCGGCAGG | |
| | CCAAAGAGCTGTTTCTTCTTCGAAAGACATTGGAGGAAATGGAGCTGCGTAT | |
| | TGAGACTCAAAAGCAGACCCTAAATGCTCGGGATGAATCCATTAAGAAGCTT | |
| | CTGGAAATGTTGCAGAGCAAAGGACTTTCTGCCAAGGCTACCGAGGAAGACC | |
| | ATGAGAGAACAAGACGACTGGCAGAGGCAGAGATGCACGTTCATCACCTAGA | |
| | AAGCCTTTTGGAGCAGAAGGAAAAAGAGAACAGTATGTTGAGAGAGGAGATG | |
| | CATCGAAGGTTTGAGAATGCTCCTGATTCTGCCAAAACAAAAGCTCTGCAAA | |
| | CTGTTATTGAGATGAAGGATTCAAAAATTTCCTCTATGGAGCGTGGGCTTCG | |
| | AGACCTGGAAGAGGAAATTCAGATGCTGAAATCGAATGGTGCTTTGAGTACT | |
| | GAGGAAAGGGAAGAAGAAATGAAGCAAATGGAAGTGTATCGGAGCCATTCTA | |
| | AATTTATGAAAAATAAGATTGGCCAGGTGAAACAGGAGCTGTCCAGAAAGGA | |
| | CACAGAACTACTCGCCCTGCAGACAAAGCTAGAAACACTCACAAACCAGTTC | |
| | TCAGATAGTAAACAGCACATTGAAGTGTTGAAGGAGTCCTTGACTGCTAAGG | |
| | AGCAGAGGGCTGCCATCCTGCAGACTGAGGTGGATGCTCTCCGATTGCGTTT | |
| | GGAAGAGAAGGAAACCATGTTGAATAAAAAGACAAAACAAATTCAGGATATG | |
| | GCTGAAGAGAAGGGGACACAAGCTGGAGAGATACATGACCTCAAGGACATGT | |
| | TGGATGTGAAGGAGCGGAAGGTTAATGTTCTTCAGAAGAAGATTGAAAATCT | |
| | TCAAGAGCAGCTTAGAGACAAGGAAAAGCAGATGAGCAGCTTGAAAGAACGG | |
| | GTCAAATCCTTGCAGGCTGACACCACCAACACTGACACTGCCTTGACAACTT | |
| | TGGAGGAGGCCCTTGCAGAGAAAGAGCGGACAATTGAACGCTTAAAGGAGCA | |
| | GAGGGACAGAGATGAGCGAGAGAAGCAAGAGGAAATTGATAACTACAAAAAA | |
| | GATCTTAAAGACTTGAAGGAAAAAGTCAGCCTGTTGCAAGGCGACCTTTCAG | |
| | AGAAAGAGGCTTCACTTTTGGATCTGAAAGAGCATGCTTCTTCTCTGGCATC | |
| | CTCAGGACTGAAAAAGGACTCACGGCTTAAGACACTAGAGATTGCTTTGGAG | |
| | CAGAAGAAGGAGGAGTGTCTGAAAATGGAATCACAATTGAAAAAGGCACATG | |
| | AGGCAGCATTGGAAGCCAGAGCCAGTCCAGAGATGAGTGACCGAATACAGCA | |
| | CTTGGAGAGAGAGATCACCAGGTACAAAGATGAATCTAGCAAGGCCCAGGCA | |
| | GAAGTTGATCGACTCTTAGAAATCTTGAAGGAGGTGGAAAATGAGAAGAATG | |
| | ACAAAGATAAGAAGATAGCTGAGTTGGAAAGGCAAGTGAAAGACCAGAATAA | |
| | GAAGGTAGCAAATCTGAAGCACAAGGAACAGGTGGAAAAAAAGAAGAGTGCA | |
| | CAAATGTTAGAGGAGGCGCGACGACGGGAGGACAATCTCAACGACAGCTCTC | |
| | AGCAGCTACAGATCCACTGTGCGACGAGCTGTGCCGCACGGTGATCGCAGCC | |
| | GCTGTCCTCTTCTCCTTCATCGTCTCGGTGCTGCTGTCTGCCTTCTGCATCC | |
| | ACTGCTACCACAAGTTTGCCCACAAGCCACCCATCTCCTCAGCTGAGATGAC | |
| | CTTCCGGAGGCCCGCCCAGGCCTTCCCGGTCAGCTACTCCTCTTCCGGTGCC | |
| | CGCCGGCCCTCGCTGGACTCCATGGAGAACCAGGTCTCCGTGGATGCCTTCA | |
| | AGATCCTGGAGGATCCAAAGTGGGAATTCCCTCGGAAGAACTTGGTTCTTGG | |
| | AAAAACTCTAGGAGAAGGCGAATTTGGAAAAGTGGTCAAGGCAACGGCCTTC | |
| | CATCTGAAAGGCAGAGCAGGGTACACCACGGTGGCCGTGAAGATGCTGAAAG | |
| | AGAACGCCTCCCCGAGTGAGCTGCGAGACCTGCTGTCAGAGTTCAACGTCCT | |
| | GAAGCAGGTCAACCACCCACATGTCATCAAATTGTATGGGGCCTGCAGCCAG | |
| | GATGGCCCGCTCCTCCTCATCGTGGAGTACGCCAAATACGGCTCCCTGCGGG | |
| | GCTTCCTCCGCGAGAGCCGCAAAGTGGGGCCTGGCTACCTGGGCAGTGGAGG | |
| | CAGCCGCAACTCCAGCTCCCTGGACCACCCGGATGAGCGGGCCCTCACCATG | |
| | GGCGACCTCATCTCATTTGCCTGGCAGATCTCACAGGGGATGCAGTATCTGG | |
| | CCGAGATGAAGCTCGTTCATCGGGACTTGGCAGCCAGAAACATCCTGGTAGC | |
| | TGAGGGGCGGAAGATGAAGATTTCGGATTTCGGCTTGTCCCGAGATGTTTAT | |
| | GAAGAGGATTCCTACGTGAAGAGGAGCCAGGGTCGGATTCCAGTTAAATGGA | |
| | TGGCAATTGAATCCCTTTTTGATCATATCTACACCACGCAAAGTGATGTATG | |
| | GTCTTTTGGTGTCCTGCTGTGGGAGATCGTGACCCTAGGGGGAAACCCCTAT | |
| | CCTGGGATTCCTCCTGAGCGGCTCTTCAACCTTCTGAAGACCGGCCACCGGA | |
| | TGGAGAGGCCAGACAACTGCAGCGAGGAGATGTACCGCCTGATGCTGCAATG | |
| | CTGGAAGCAGGAGCCGGACAAAAGGCCGGTGTTTGCGGACATCAGCAAAGAC | |
| | CTGGAGAAGATGATGGTTAAGAGGAGAGACTACTTGGACCTTGCGGCGTCCA | |
| | CTCCATCTGACTCCCTGATTTATGACGACGGCCTCTCAGAGGAGGAGACACC | |
| | GCTGGTGGACTGTAATAATGCCCCCCTCCCTCGAGCCCTCCCTTCCACATGG | |
| | ATTGAAAACAAACTCTATGGTAGAATTTCCCATGCATTTACTAGATTCTAG | |
| FRMD4A- | ATGGCAGTGCAGCTGGTGCCCGACTCAGCTCTCGGCCTGCTGATGATGACGG | 46 |
| RET | AGGGCCGCCGATGTCAAGTACATCTTCTTGATGACAGGAAGCTGGAACTCCT | |
| | AGTACAGCCCAAGCTGTTGGCCAAGGAGCTTCTTGACCTTGTGGCTTCTCAC | |
| | TTCAATCTGAAGGAAAAGGAGTACTTTGGAATAGCATTCACAGATGAAACGG | |
| | GACACTTAAACTGGCTTCAGCTAGATCGAAGAGTATTGGAACATGACTTCCC | |
| | TAAAAAGTCAGGACCCGTGGTTTTATACTTTTGTGTCAGGTTCTATATAGAA | |
| | AGCATTTCATACCTGAAGGATAATGCTACCATTGAGCTTTTCTTTCTGAACG | |
| | CGAAGTCCTGCATCTACAAGGAGCTTATTGACGTTGACAGCGAAGTGGTGTT | |
| | TGAATTAGCTTCCTATATTTTACAGGAGGCAAAGGGAGATTTTTCTAGCAAT | |
| | GAAGTTGTGAGGAGTGACTTGAAGAAGCTGCCAGCCCTTCCCACCCAAGCCC | |
| | TGAAGGAGCACCCTTCCCTGGCCTACTGTGAAGACAGAGTCATTGAGCACTA | |
| | CAAGAAACTGAACGGTCAGACAAGAGGTCAAGCAATCGTAAACTACATGAGC | |
| | ATCGTGGAGTCTCTCCCAACCTACGGGGTTCACTATTATGCAGTGAAGGACA | |
| | AGCAGGGCATACCATGGTGGCTGGGCCTGAGCTACAAAGGGATCTTCCAGTA | |
| | TGACTACCATGATAAAGTGAAGCCAAGAAAGGAGGATCCAAAGTGGGAATTC | |
| | CCTCGGAAGAACTTGGTTCTTGGAAAAACTCTAGGAGAAGGCGAATTTGGAA | |
| | AAGTGGTCAAGGCAACGGCCTTCCATCTGAAAGGCAGAGCAGGGTACACCAC | |
| | GGTGGCCGTGAAGATGCTGAAAGAGAACGCCTCCCCGAGTGAGCTGCGAGAC | |
| | CTGCTGTCAGAGTTCAACGTCCTGAAGCAGGTCAACCACCCACATGTCATCA | |
| | AATTGTATGGGGCCTGCAGCCAGGATGGCCCGCTCCTCCTCATCGTGGAGTA | |
| | CGCCAAATACGGCTCCCTGCGGGGCTTCCTCCGCGAGAGCCGCAAAGTGGGG | |
| | CCTGGCTACCTGGGCAGTGGAGGCAGCCGCAACTCCAGCTCCCTGGACCACC | |
| | CGGATGAGCGGGCCCTCACCATGGGCGACCTCATCTCATTTGCCTGGCAGAT | |
| | CTCACAGGGGATGCAGTATCTGGCCGAGATGAAGCTCGTTCATCGGGACTTG | |
| | GCAGCCAGAAACATCCTGGTAGCTGAGGGGCGGAAGATGAAGATTTCGGATT | |
| | TCGGCTTGTCCCGAGATGTTTATGAAGAGGATTCCTACGTGAAGAGGAGCCA | |
| | GGGTCGGATTCCAGTTAAATGGATGGCAATTGAATCCCTTTTTGATCATATC | |
| | TACACCACGCAAAGTGATGTATGGTCTTTTGGTGTCCTGCTGTGGGAGATCG | |
| | TGACCCTAGGGGGAAACCCCTATCCTGGGATTCCTCCTGAGCGGCTCTTCAA | |
| | CCTTCTGAAGACCGGCCACCGGATGGAGAGGCCAGACAACTGCAGCGAGGAG | |
| | ATGTACCGCCTGATGCTGCAATGCTGGAAGCAGGAGCCGGACAAAAGGCCGG | |
| | TGTTTGCGGACATCAGCAAAGACCTGGAGAAGATGATGGTTAAGAGGAGAGA | |
| | CTACTTGGACCTTGCGGCGTCCACTCCATCTGACTCCCTGATTTATGACGAC | |
| | GGCCTCTCAGAGGAGGAGACACCGCTGGTGGACTGTAATAATGCCCCCCTCC | |
| | CTCGAGCCCTCCCTTCCACATGGATTGAAAACAAACTCTATGGTAGAATTTC | |
| | CCATGCATTTACTAGATTCTAG | |
| KIAA1468- | ATGGCGGCGATGGCGCCTGGAGGTAGTGGCAGTGGTGGCGGCGTGAATCCAT | 47 |
| RET | TTCTCAGTGATTCGGATGAGGACGATGACGAGGTAGCTGCAACAGAGGAACG | |
| | GCGGGCAGTACTTCGGCTGGGCGCCGGAAGTGGCCTAGATCCTGGCTCTGCG | |
| | GGCTCGCTGTCGCCACAGGATCCCGTGGCCTTAGGAAGCAGTGCGCGGCCAG | |
| | GGCTCCCTGGGGAGGCGTCGGCGGCTGCAGTGGCCCTGGGGGGCACCGGGGA | |
| | GACCCCGGCCCGATTATCAATTGATGCGATCGCTGCTCAGCTGTTGCGCGAT | |
| | CAATACTTGCTGACCGCCCTGGAGCTGCATACCGAGCTGTTAGAGAGTGGCC | |
| | GGGAGCTGCCTCGGCTGCGCGACTACTTCTCCAATCCAGGCAACTTCGAGAG | |
| | GCAAAGTGGAACCCCGCCGGGGATGGGGGCGCCAGGGGTCCCTGGAGCAGCC | |
| | GGCGTTGGGGGCGCTGGAGGTCGGGAACCGAGTACAGCGTCGGGGGGGGAC | |
| | AGCTCAATCGAGCTGGGAGCATTAGTACCCTTGATTCTTTAGACTTTGCAAG | |
| | ATATTCAGATGATGGTAACAGGGAAACAGATGAAAAAGTGGCAGTCCTGGAG | |
| | TTTGAACTACGGAAAGCCAAGGAGACCATTCAGGCCCTCCGAGCCAACCTGA | |
| | CAAAGGCCGCAGAACATGAAGTTCCTTTACAGGAACGAAAAAATTACAAATC | |
| | AAGTCCTGAAATTCAGGAGCCAATCAAACCTCTTGAAAAGAGAGCTCTAAAC | |
| | TTCTTAGTCAATGAATTTTTATTGAAGAATAACTATAAGCTTACATCAATAA | |
| | CCTTTTCAGATGAAAACGATGATCAGGATTTTGAATTATGGGATGATGTAGG | |
| | ATTAAACATTCCAAAACCTCCAGACTTATTGCAACTCTACCGGGATTTTGGA | |
| | AATCATCAAGTAACTGGAAAAGATCTTGTAGATGTGGCCAGTGGAGTAGAAG | |
| | AAGATGAATTAGAGGCCCTTACACCAATTATAAGCAACCTTCCTCCAACTCT | |
| | TGAAACTCCCCAGCCTGCAGAGAACTCCATGTTAGTACAGAAATTAGAAGAT | |
| | AAAATTAGTTTGTTAAATAGTGAGAAATGGTCATTGATGGAGCAAATCAGAA | |
| | GACTTAAAAGTGAAATGGACTTCCTCAAAAATGAACACTTTGCCATCCCAGC | |
| | AGTTTGTGACTCTGTTCAGCCTCCTTTGGATCAGTTGCCCCACAAAGACTCT | |
| | GAGGACAGTGGACAGCATCCAGATGTAAATAGTTCAGACAAGGGAAAAAACA | |
| | CAGACATCCATCTTTCAATATCAGATGAAGCTGATTCCACTATTCCTAAAGA | |
| | GAATTCCCCAAATTCATTCCCCAGGAGAGAAAGAGAAGGAATGCCACCTTCT | |
| | TCTCTATCAAGTAAAAAGACAGTTCATTTTGATAAACCTAATAGGAAATTGT | |
| | CTCCTGCATTCCATCAAGCACTACTCTCTTTTTGTCGAATGTCAGCAGATAG | |
| | TCGTTTAGGATACGAGGTGTCTCGTATTGCAGACAGTGAAAAAAGCGTTATG | |
| | TTAATGCTGGGACGCTGCCTGCCACACATTGTTCCCAATGTGCTATTGGCAA | |
| | AGAGAGAGGAGGATCCAAAGTGGGAATTCCCTCGGAAGAACTTGGTTCTTGG | |
| | AAAAACTCTAGGAGAAGGCGAATTTGGAAAAGTGGTCAAGGCAACGGCCTTC | |
| | CATCTGAAAGGCAGAGCAGGGTACACCACGGTGGCCGTGAAGATGCTGAAAG | |
| | AGAACGCCTCCCCGAGTGAGCTGCGAGACCTGCTGTCAGAGTTCAACGTCCT | |
| | GAAGCAGGTCAACCACCCACATGTCATCAAATTGTATGGGGCCTGCAGCCAG | |
| | GATGGCCCGCTCCTCCTCATCGTGGAGTACGCCAAATACGGCTCCCTGCGGG | |
| | GCTTCCTCCGCGAGAGCCGCAAAGTGGGGCCTGGCTACCTGGGCAGTGGAGG | |
| | CAGCCGCAACTCCAGCTCCCTGGACCACCCGGATGAGCGGGCCCTCACCATG | |
| | GGCGACCTCATCTCATTTGCCTGGCAGATCTCACAGGGGATGCAGTATCTGG | |
| | CCGAGATGAAGCTCGTTCATCGGGACTTGGCAGCCAGAAACATCCTGGTAGC | |
| | TGAGGGGCGGAAGATGAAGATTTCGGATTTCGGCTTGTCCCGAGATGTTTAT | |
| | GAAGAGGATTCCTACGTGAAGAGGAGCCAGGGTCGGATTCCAGTTAAATGGA | |
| | TGGCAATTGAATCCCTTTTTGATCATATCTACACCACGCAAAGTGATGTATG | |
| | GTCTTTTGGTGTCCTGCTGTGGGAGATCGTGACCCTAGGGGGAAACCCCTAT | |
| | CCTGGGATTCCTCCTGAGCGGCTCTTCAACCTTCTGAAGACCGGCCACCGGA | |
| | TGGAGAGGCCAGACAACTGCAGCGAGGAGATGTACCGCCTGATGCTGCAATG | |
| | CTGGAAGCAGGAGCCGGACAAAAGGCCGGTGTTTGCGGACATCAGCAAAGAC | |
| | CTGGAGAAGATGATGGTTAAGAGGAGAGACTACTTGGACCTTGCGGCGTCCA | |
| | CTCCATCTGACTCCCTGATTTATGACGACGGCCTCTCAGAGGAGGAGACACC | |
| | GCTGGTGGACTGTAATAATGCCCCCCTCCCTCGAGCCCTCCCTTCCACATGG | |
| | ATTGAAAACAAACTCTATGGTAGAATTTCCCATGCATTTACTAGATTCTAG | |
| NCOA4- | ATGAATACCTTCCAAGACCAGAGTGGCAGCTCCAGTAATAGAGAACCCCTTT | 48 |
| RET | TGAGGTGTAGTGATGCACGGAGGGACTTGGAGCTTGCTATTGGTGGAGTTCT | |
| | CCGGGCTGAACAGCAAATTAAAGATAACTTGCGAGAGGTCAAAGCTCAGATT | |
| | CACAGTTGCATAAGCCGTCACCTGGAATGTCTTAGAAGCCGTGAGGTATGGC | |
| | TGTATGAACAGGTGGACCTTATTTATCAGCTTAAAGAGGAGACACTTCAACA | |
| | GCAGGCTCAGCAGCTCTACTCGTTATTGGGCCAGTTCAATTGTCTTACTCAT | |
| | CAACTGGAGTGTACCCAAAACAAAGATCTAGCCAATCAAGTCTCTGTGTGCC | |
| | TGGAGAGACTGGGCAGTTTGACCCTTAAGCCTGAAGATTCAACTGTCCTGCT | |
| | CTTTGAAGCTGACACAATTACTCTGCGCCAGACCATCACCACATTTGGGTCT | |
| | CTCAAAACCATTCAAATTCCTGAGCACTTGATGGCTCATGCTAGTTCAGCAA | |
| | ATATTGGGCCCTTCCTGGAGAAGAGAGGCTGTATCTCCATGCCAGAGCAGAA | |
| | GTCAGCATCCGGTATTGTAGCTGTCCCTTTCAGCGAATGGCTCCTTGGAAGC | |
| | AAACCTGCCAGTGGTTATCAAGCTCCTTACATACCCAGCACCGACCCCCAGG | |
| | ACTGGCTTACCCAAAAGCAGACCTTGGAGAACAGTCAGACTTCTTCCAGAGC | |
| | CTGCAATTTCTTCAATAATGTCGGGGGAAACCTAAAGGGCTTAGAAAACTGG | |
| | CTCCTCAAGAGTGAAAAATCAAGTTATCAAAAGTGTAACAGCCATTCCACTA | |
| | CTAGTTCTTTCTCCATTGAAATGGAAAAGGTTGGAGATCAAGAGCTTCCTGA | |
| | TCAAGATGAGATGGACCTATCAGATTGGCTAGTGACTCCCCAGGAATCCCAT | |
| | AAGCTGCGGAAGCCTGAGAATGGCAGTCGTGAAACCAGTGAGAAGTTTAAGC | |
| | TCTTATTCCAGTCCTATAATGTGAATGATTGGCTTGTCAAGACTGACTCCTG | |
| | TACCAACTGTCAGGGAAACCAGCCCAAAGGTGTGGAGATTGAAAACCTGGGC | |
| | AATCTGAAGTGCCTGAATGACCACTTGGAGGCCAAGAAACCATTGTCCACCC | |
| | CCAGCATGGTTACAGAGGATTGGCTTGTCCAGAACCATCAGGACCCATGTAA | |
| | GGTAGAGGAGGTGTGCAGAGCCAATGAGCCCTGCACAAGCTTTGCAGAGTGT | |
| | GTGTGTGATGAGAATTGTGAGAAGGAGGCTCTGTATAAGTGGCTTCTGAAGA | |
| | AAGAAGGAAAGGATAAAAATGGGATGCCTGTGGAACCCAAACCTGAGCCTGA | |
| | GAAGCATAAAGATTCCCTGAATATGTGGCTCTGTCCTAGAAAAGAAGTAATA | |
| | GAACAAACTAAAGCACCAAAGGCAATGACTCCTTCTAGAATTGCTGATTCCT | |
| | TCCAAGTCATAAAGAACAGCCCCTTGTCGGAGTGGCTTATCAGGCCCCCATA | |
| | CAAAGAAGGAAGTCCCAAGGAAGTGCCTGGTACTGAAGACAGAGCTGGCAAA | |
| | CAGAAGTTTAAAAGCCCCATGAATACTTCCTGGTGTTCCTTTAACACAGCTG | |
| | ACTGGGTCCTGCCAGGAAAGAAGATGGGCAACCTCAGCCAGTTATCTTCTGG | |
| | AGAAGACAAGTGGCTGCTTCGAAAGAAGGCCCAGGAGGATCCAAAGTGGGAA | |
| | TTCCCTCGGAAGAACTTGGTTCTTGGAAAAACTCTAGGAGAAGGCGAATTTG | |
| | GAAAAGTGGTCAAGGCAACGGCCTTCCATCTGAAAGGCAGAGCAGGGTACAC | |
| | CACGGTGGCCGTGAAGATGCTGAAAGAGAACGCCTCCCCGAGTGAGCTGCGA | |
| | GACCTGCTGTCAGAGTTCAACGTCCTGAAGCAGGTCAACCACCCACATGTCA | |
| | TCAAATTGTATGGGGCCTGCAGCCAGGATGGCCCGCTCCTCCTCATCGTGGA | |
| | GTACGCCAAATACGGCTCCCTGCGGGGCTTCCTCCGCGAGAGCCGCAAAGTG | |
| | GGGCCTGGCTACCTGGGCAGTGGAGGCAGCCGCAACTCCAGCTCCCTGGACC | |
| | ACCCGGATGAGCGGGCCCTCACCATGGGCGACCTCATCTCATTTGCCTGGCA | |
| | GATCTCACAGGGGATGCAGTATCTGGCCGAGATGAAGCTCGTTCATCGGGAC | |
| | TTGGCAGCCAGAAACATCCTGGTAGCTGAGGGGCGGAAGATGAAGATTTCGG | |
| | ATTTCGGCTTGTCCCGAGATGTTTATGAAGAGGATTCCTACGTGAAGAGGAG | |
| | CCAGGGTCGGATTCCAGTTAAATGGATGGCAATTGAATCCCTTTTTGATCAT | |
| | ATCTACACCACGCAAAGTGATGTATGGTCTTTTGGTGTCCTGCTGTGGGAGA | |
| | TCGTGACCCTAGGGGGAAACCCCTATCCTGGGATTCCTCCTGAGCGGCTCTT | |
| | CAACCTTCTGAAGACCGGCCACCGGATGGAGAGGCCAGACAACTGCAGCGAG | |
| | GAGATGTACCGCCTGATGCTGCAATGCTGGAAGCAGGAGCCGGACAAAAGGC | |
| | CGGTGTTTGCGGACATCAGCAAAGACCTGGAGAAGATGATGGTTAAGAGGAG | |
| | AGACTACTTGGACCTTGCGGCGTCCACTCCATCTGACTCCCTGATTTATGAC | |
| | GACGGCCTCTCAGAGGAGGAGACACCGCTGGTGGACTGTAATAATGCCCCCC | |
| | TCCCTCGAGCCCTCCCTTCCACATGGATTGAAAACAAACTCTATGGTAGAAT | |
| | TTCCCATGCATTTACTAGATTCTAG | |
| PARD3- | ATGAAAGTGACCGTGTGCTTCGGACGGACCCGGGTGGTCGTGCCGTGCGGGG | 49 |
| RET | ACGGCCACATGAAAGTTTTCAGCCTCATCCAGCAGGCGGTGACCCGCTACCG | |
| | GAAGGCCATCGCCAAGGATCCAAACTACTGGATACAGGTGCATCGCTTGGAA | |
| | CATGGAGATGGAGGAATACTAGACCTTGATGACATTCTTTGTGATGTAGCAG | |
| | ACGATAAAGACAGAATGTGGCCGAGGAGGCGGGCTGCCCCCTGTCCTGTGCA | |
| | GTCAGCAAGAGACGGCTGGAGTGTGAGGAGTGTGGCGGCCTGGGCTCCCCAA | |
| | CAGGCAGGTGTGAGTGGAGGCAAGGAGATGGCAAAGGGATCACCAGGAACTT | |
| | CTCCACCTGCTCTCCCAGCACCAAGACCTGCCCCGACGGCCACTGCGATGTT | |
| | GTGGAGACCCAAGACATCAACATTTGCCCTCAGGACTGCCTCCGGGGCAGCA | |
| | TTGTTGGGGGACACGAGCCTGGGGAGCCCCGGGGGATTAAAGCTGGCTATGG | |
| | CACCTGCAACTGCTTCCCTGAGGAGGAGAAGTGCTTCTGCGAGCCCGAAGAC | |
| | ATCCAGGATCCACTGTGCGACGAGCTGTGCCGCACGGTGATCGCAGCCGCTG | |
| | TCCTCTTCTCCTTCATCGTCTCGGTGCTGCTGTCTGCCTTCTGCATCCACTG | |
| | CTACCACAAGTTTGCCCACAAGCCACCCATCTCCTCAGCTGAGATGACCTTC | |
| | CGGAGGCCCGCCCAGGCCTTCCCGGTCAGCTACTCCTCTTCCGGTGCCCGCC | |
| | GGCCCTCGCTGGACTCCATGGAGAACCAGGTCTCCGTGGATGCCTTCAAGAT | |
| | CCTGGAGGATCCAAAGTGGGAATTCCCTCGGAAGAACTTGGTTCTTGGAAAA | |
| | ACTCTAGGAGAAGGCGAATTTGGAAAAGTGGTCAAGGCAACGGCCTTCCATC | |
| | TGAAAGGCAGAGCAGGGTACACCACGGTGGCCGTGAAGATGCTGAAAGAGAA | |
| | CGCCTCCCCGAGTGAGCTGCGAGACCTGCTGTCAGAGTTCAACGTCCTGAAG | |
| | CAGGTCAACCACCCACATGTCATCAAATTGTATGGGGCCTGCAGCCAGGATG | |
| | GCCCGCTCCTCCTCATCGTGGAGTACGCCAAATACGGCTCCCTGCGGGGCTT | |
| | CCTCCGCGAGAGCCGCAAAGTGGGGCCTGGCTACCTGGGCAGTGGAGGCAGC | |
| | CGCAACTCCAGCTCCCTGGACCACCCGGATGAGCGGGCCCTCACCATGGGCG | |
| | ACCTCATCTCATTTGCCTGGCAGATCTCACAGGGGATGCAGTATCTGGCCGA | |
| | GATGAAGCTCGTTCATCGGGACTTGGCAGCCAGAAACATCCTGGTAGCTGAG | |
| | GGGCGGAAGATGAAGATTTCGGATTTCGGCTTGTCCCGAGATGTTTATGAAG | |
| | AGGATTCCTACGTGAAGAGGAGCCAGGGTCGGATTCCAGTTAAATGGATGGC | |
| | AATTGAATCCCTTTTTGATCATATCTACACCACGCAAAGTGATGTATGGTCT | |
| | TTTGGTGTCCTGCTGTGGGAGATCGTGACCCTAGGGGGAAACCCCTATCCTG | |
| | GGATTCCTCCTGAGCGGCTCTTCAACCTTCTGAAGACCGGCCACCGGATGGA | |
| | GAGGCCAGACAACTGCAGCGAGGAGATGTACCGCCTGATGCTGCAATGCTGG | |
| | AAGCAGGAGCCGGACAAAAGGCCGGTGTTTGCGGACATCAGCAAAGACCTGG | |
| | AGAAGATGATGGTTAAGAGGAGAGACTACTTGGACCTTGCGGCGTCCACTCC | |
| | ATCTGACTCCCTGATTTATGACGACGGCCTCTCAGAGGAGGAGACACCGCTG | |
| | GTGGACTGTAATAATGCCCCCCTCCCTCGAGCCCTCCCTTCCACATGGATTG | |
| | AAAACAAACTCTATGGTAGAATTTCCCATGCATTTACTAGATTCTAG | |
| PARD3- | ATGAAAGTGACCGTGTGCTTCGGACGGACCCGGGTGGTCGTGCCGTGCGGGG | 50 |
| RET | ACGGCCACATGAAAGTTTTCAGCCTCATCCAGCAGGCGGTGACCCGCTACCG | |
| | GAAGGCCATCGCCAAGGATCCAAACTACTGGATACAGGTGCATCGCTTGGAA | |
| | CATGGAGATGGAGGAATACTAGACCTTGATGACATTCTTTGTGATGTAGCAG | |
| | ACGATAAAGACAGACTGGTAGCAGTGTTTGATGAGCAGGATCCACATCACGG | |
| | AGGTGATGGCACCAGTGCCAGTTCCACGGGTACCCAGAGCCCAGAGATATTT | |
| | GGTAGTGAGCTTGGCACCAACAATGTCTCAGCCTTTCAGCCTTACCAAGCAA | |
| | CAAGTGAAATTGAGGTCACACCTTCAGTCCTTCGAGCAAATATGCCTCTTCA | |
| | TGTTCGACGCAGTAGTGACCCAGCTCTAATTGGCCTCTCCACTTCTGTCAGT | |
| | GATAGTAATTTTTCCTCTGAAGAGCCTTCAAGGAAAAATCCCACACGCTGGT | |
| | CAACAACAGCTGGCTTCCTCAAGCAGAACACTGCTGGGAGTCCTAAAACCTG | |
| | CGACAGGAAGGAGGATCCAAAGTGGGAATTCCCTCGGAAGAACTTGGTTCTT | |
| | GGAAAAACTCTAGGAGAAGGCGAATTTGGAAAAGTGGTCAAGGCAACGGCCT | |
| | TCCATCTGAAAGGCAGAGCAGGGTACACCACGGTGGCCGTGAAGATGCTGAA | |
| | AGAGAACGCCTCCCCGAGTGAGCTGCGAGACCTGCTGTCAGAGTTCAACGTC | |
| | CTGAAGCAGGTCAACCACCCACATGTCATCAAATTGTATGGGGCCTGCAGCC | |
| | AGGATGGCCCGCTCCTCCTCATCGTGGAGTACGCCAAATACGGCTCCCTGCG | |
| | GGGCTTCCTCCGCGAGAGCCGCAAAGTGGGGCCTGGCTACCTGGGCAGTGGA | |
| | GGCAGCCGCAACTCCAGCTCCCTGGACCACCCGGATGAGCGGGCCCTCACCA | |
| | TGGGCGACCTCATCTCATTTGCCTGGCAGATCTCACAGGGGATGCAGTATCT | |
| | GGCCGAGATGAAGCTCGTTCATCGGGACTTGGCAGCCAGAAACATCCTGGTA | |
| | GCTGAGGGGCGGAAGATGAAGATTTCGGATTTCGGCTTGTCCCGAGATGTTT | |
| | ATGAAGAGGATTCCTACGTGAAGAGGAGCCAGGGTCGGATTCCAGTTAAATG | |
| | GATGGCAATTGAATCCCTTTTTGATCATATCTACACCACGCAAAGTGATGTA | |
| | TGGTCTTTTGGTGTCCTGCTGTGGGAGATCGTGACCCTAGGGGGAAACCCCT | |
| | ATCCTGGGATTCCTCCTGAGCGGCTCTTCAACCTTCTGAAGACCGGCCACCG | |
| | GATGGAGAGGCCAGACAACTGCAGCGAGGAGATGTACCGCCTGATGCTGCAA | |
| | TGCTGGAAGCAGGAGCCGGACAAAAGGCCGGTGTTTGCGGACATCAGCAAAG | |
| | ACCTGGAGAAGATGATGGTTAAGAGGAGAGACTACTTGGACCTTGCGGCGTC | |
| | CACTCCATCTGACTCCCTGATTTATGACGACGGCCTCTCAGAGGAGGAGACA | |
| | CCGCTGGTGGACTGTAATAATGCCCCCCTCCCTCGAGCCCTCCCTTCCACAT | |
| | GGATTGAAAACAAACTCTATGGTAGAATTTCCCATGCATTTACTAGATTCTA | |
| | G | |
| PRKAR1A- | ATGGAGTCTGGCAGTACCGCCGCCAGTGAGGAGGCACGCAGCCTTCGAGAAT | 51 |
| RET | GTGAGCTCTACGTCCAGAAGCATAACATTCAAGCGCTGCTCAAAGATTCTAT | |
| | TGTGCAGTTGTGCACTGCTCGACCTGAGAGACCCATGGCATTCCTCAGGGAA | |
| | TACTTTGAGAGGTTGGAGAAGGAGGAGGCAAAACAGATTCAGAATCTGCAGA | |
| | AAGCAGGCACTCGTACAGACTCAAGGGAGGATGAGATTTCTCCTCCTCCACC | |
| | CAACCCAGTGGTTAAAGGTAGGAGGCGACGAGGTGCTATCAGCGCTGAGGTC | |
| | TACACGGAGGAAGATGCGGCATCCTATGTTAGAAAGGTTATACCAAAAGATT | |
| | ACAAGACAATGGCCGCTTTAGCCAAAGCCATTGAAAAGAATGTGCTGTTTTC | |
| | ACATCTTGATGATAATGAGAGAAGTGATATTTTTGATGCCATGTTTTCGGTC | |
| | TCCTTTATCGCAGGAGAGACTGTGATTCAGCAAGGTGATGAAGGGGATAACT | |
| | TCTATGTGATTGATCAAGGAGAGACGGATGTCTATGTTAACAATGAATGGGC | |
| | AACCAGTGTTGGGGAAGGAGGGAGCTTTGGAGAACTTGCTTTGATTTATGGA | |
| | ACACCGAGAGCAGCCACTGTCAAAGCAAAGACAAATGTGAAATTGTGGGGCA | |
| | TCGACCGAGACAGCTATAGAAGAATCCTCATGGAGGATCCAAAGTGGGAATT | |
| | CCCTCGGAAGAACTTGGTTCTTGGAAAAACTCTAGGAGAAGGCGAATTTGGA | |
| | AAAGTGGTCAAGGCAACGGCCTTCCATCTGAAAGGCAGAGCAGGGTACACCA | |
| | CGGTGGCCGTGAAGATGCTGAAAGAGAACGCCTCCCCGAGTGAGCTGCGAGA | |
| | CCTGCTGTCAGAGTTCAACGTCCTGAAGCAGGTCAACCACCCACATGTCATC | |
| | AAATTGTATGGGGCCTGCAGCCAGGATGGCCCGCTCCTCCTCATCGTGGAGT | |
| | ACGCCAAATACGGCTCCCTGCGGGGCTTCCTCCGCGAGAGCCGCAAAGTGGG | |
| | GCCTGGCTACCTGGGCAGTGGAGGCAGCCGCAACTCCAGCTCCCTGGACCAC | |
| | CCGGATGAGCGGGCCCTCACCATGGGCGACCTCATCTCATTTGCCTGGCAGA | |
| | TCTCACAGGGGATGCAGTATCTGGCCGAGATGAAGCTCGTTCATCGGGACTT | |
| | GGCAGCCAGAAACATCCTGGTAGCTGAGGGGCGGAAGATGAAGATTTCGGAT | |
| | TTCGGCTTGTCCCGAGATGTTTATGAAGAGGATTCCTACGTGAAGAGGAGCC | |
| | AGGGTCGGATTCCAGTTAAATGGATGGCAATTGAATCCCTTTTTGATCATAT | |
| | CTACACCACGCAAAGTGATGTATGGTCTTTTGGTGTCCTGCTGTGGGAGATC | |
| | GTGACCCTAGGGGGAAACCCCTATCCTGGGATTCCTCCTGAGCGGCTCTTCA | |
| | ACCTTCTGAAGACCGGCCACCGGATGGAGAGGCCAGACAACTGCAGCGAGGA | |
| | GATGTACCGCCTGATGCTGCAATGCTGGAAGCAGGAGCCGGACAAAAGGCCG | |
| | GTGTTTGCGGACATCAGCAAAGACCTGGAGAAGATGATGGTTAAGAGGAGAG | |
| | ACTACTTGGACCTTGCGGCGTCCACTCCATCTGACTCCCTGATTTATGACGA | |
| | CGGCCTCTCAGAGGAGGAGACACCGCTGGTGGACTGTAATAATGCCCCCCTC | |
| | CCTCGAGCCCTCCCTTCCACATGGATTGAAAACAAACTCTATGGTAGAATTT | |
| | CCCATGCATTTACTAGATTCTAG | |
| PRKG1- | ATGGGCACCTTGCGGGATTTACAGTACGCGCTCCAGGAGAAGATCGAGGAGC | 52 |
| RET | TGAGGCAGCGGGATGCTCTCATCGACGAGCTGGAGCTGGAGTTGGATCAGAA | |
| | GGACGAACTGATCCAGAAGCTGCAGAACGAGCTGGACAAGTACCGCTCGGTG | |
| | ATCCGACCAGCCACCCAGCAGGCGCAGAAGCAGAGCGCGAGCACCTTGCAGG | |
| | GCGAGCCGCGCACCAAGCGGCAGGCGATCTCCGCCGAGCCCACCGCCTTCGA | |
| | CATCCAGGATCTCAGCCATGTGACCCTGCCCTTCTACCCCAAGAGCCCACAG | |
| | TCCAAGGATCTTATAAAGGAAGCTATCCTTGACAATGACTTTATGAAGAACT | |
| | TGGAGCTGTCGCAGATCCAGGAGATTGTGGATTGTATGTACCCGGTGGAGTA | |
| | TGGCAAGGACAGTTGCATCATCAAAGAAGGAGACGTGGGGTCACTGGTGTAT | |
| | GTCATGGAAGATGGTAAGGTTGAAGTTACAAAAGAAGGTGTGAAGTTGTGTA | |
| | CCATGGGTCCAGGAAAAGTGTTTGGGGAATTGGCTATTCTTTACAACTGTAC | |
| | CCGGACAGCGACCGTCAAGACTCTTGTAAATGTAAAACTCTGGGCCATTGAT | |
| | CGACAATGTTTTCAAACAATAATGATGAGGACAGGACTCATCAAGCATACCG | |
| | AGTATATGGAATTTTTAAAAAGCGTTCCAACATTCCAGAGCCTTCCTGAAGA | |
| | GATCCTCAGCAAGCTTGCTGATGTCCTTGAAGAGACCCACTATGAAAATGGA | |
| | GAATATATTATCAGGCAAGGTGCAAGAGGGGACACCTTCTTTATCATCAGCA | |
| | AAGGAACGGTAAATGTCACTCGTGAAGACTCACCGAGTGAAGACCCAGTCTT | |
| | TCTTAGAACTTTAGGAAAAGGAGACTGGTTTGGAGAGAAAGCCTTGCAGGGG | |
| | TGAGGGTCTGCCCTTCCGCTGCGCCCCGGACAGCCTGGAGGTGAGCACGCGC | |
| | TGGGCCCTGGACCGCGAGCAGCGGGAGAAGTACGAGCTGGTGGCCGTGTGCA | |
| | CCGTGCACGCCGGCGCGCGCGAGGAGGTGGTGATGGTGCCCTTCCCGGTGAC | |
| | CGTGTACGACGAGGACGACTCGGCGCCCACCTTCCCCGCGGGCGTCGACACC | |
| | GCCAGCGCCGTGGTGGAGTTCAAGCGGAAGGAGGACACCGTGGTGGCCACGC | |
| | TGCGTGTCTTCGATGCAGACGTGGTACCTGCATCAGGGGAGCTGGTGAGGCG | |
| | GTACACAAGCACGCTGCTCCCCGGGGACACCTGGGCCCAGCAGACCTTCCGG | |
| | GTGGAACACTGGCCCAACGAGACCTCGGTCCAGGCCAACGGCAGCTTCGTGC | |
| | GGGCGACCGTACATGACTATAGGCTGGTTCTCAACCGGAACCTCTCCATCTC | |
| | GGAGAACCGCACCATGCAGCTGGCGGTGCTGGTCAATGACTCAGACTTCCAG | |
| | GGCCCAGGAGCGGGCGTCCTCTTGCTCCACTTCAACGTGTCGGTGCTGCCGG | |
| | TCAGCCTGCACCTGCCCAGTACCTACTCCCTCTCCGTGAGCAGGAGGGCTCG | |
| | CCGATTTGCCCAGATCGGGAAAGTCTGTGTGGAAAACTGCCAGGCATTCAGT | |
| | GGCATCAACGTCCAGTACAAGCTGCATTCCTCTGGTGCCAACTGCAGCACGC | |
| | TAGGGGTGGTCACCTCAGCCGAGGACACCTCGGGGATCCTGTTTGTGAATGA | |
| | CACCAAGGCCCTGCGGCGGCCCAAGTGTGCCGAACTTCACTACATGGTGGTG | |
| | GCCACCGACCAGCAGACCTCTAGGCAGGCCCAGGCCCAGCTGCTTGTAACAG | |
| | TGGAGGGGTCATATGTGGCCGAGGAGGCGGGCTGCCCCCTGTCCTGTGCAGT | |
| | CAGCAAGAGACGGCTGGAGTGTGAGGAGTGTGGCGGCCTGGGCTCCCCAACA | |
| | GGCAGGTGTGAGTGGAGGCAAGGAGATGGCAAAGGGATCACCAGGAACTTCT | |
| | CCACCTGCTCTCCCAGCACCAAGACCTGCCCCGACGGCCACTGCGATGTTGT | |
| | GGAGACCCAAGACATCAACATTTGCCCTCAGGACTGCCTCCGGGGCAGCATT | |
| | GTTGGGGGACACGAGCCTGGGGAGCCCCGGGGGATTAAAGCTGGCTATGGCA | |
| | CCTGCAACTGCTTCCCTGAGGAGGAGAAGTGCTTCTGCGAGCCCGAAGACAT | |
| | CCAGGATCCACTGTGCGACGAGCTGTGCCGCACGGTGATCGCAGCCGCTGTC | |
| | CTCTTCTCCTTCATCGTCTCGGTGCTGCTGTCTGCCTTCTGCATCCACTGCT | |
| | ACCACAAGTTTGCCCACAAGCCACCCATCTCCTCAGCTGAGATGACCTTCCG | |
| | GAGGCCCGCCCAGGCCTTCCCGGTCAGCTACTCCTCTTCCGGTGCCCGCCGG | |
| | CCCTCGCTGGACTCCATGGAGAACCAGGTCTCCGTGGATGCCTTCAAGATCC | |
| | TGGAGGATCCAAAGTGGGAATTCCCTCGGAAGAACTTGGTTCTTGGAAAAAC | |
| | TCTAGGAGAAGGCGAATTTGGAAAAGTGGTCAAGGCAACGGCCTTCCATCTG | |
| | AAAGGCAGAGCAGGGTACACCACGGTGGCCGTGAAGATGCTGAAAGAGAACG | |
| | CCTCCCCGAGTGAGCTGCGAGACCTGCTGTCAGAGTTCAACGTCCTGAAGCA | |
| | GGTCAACCACCCACATGTCATCAAATTGTATGGGGCCTGCAGCCAGGATGGC | |
| | CCGCTCCTCCTCATCGTGGAGTACGCCAAATACGGCTCCCTGCGGGGCTTCC | |
| | TCCGCGAGAGCCGCAAAGTGGGGCCTGGCTACCTGGGCAGTGGAGGCAGCCG | |
| | CAACTCCAGCTCCCTGGACCACCCGGATGAGCGGGCCCTCACCATGGGCGAC | |
| | CTCATCTCATTTGCCTGGCAGATCTCACAGGGGATGCAGTATCTGGCCGAGA | |
| | TGAAGCTCGTTCATCGGGACTTGGCAGCCAGAAACATCCTGGTAGCTGAGGG | |
| | GCGGAAGATGAAGATTTCGGATTTCGGCTTGTCCCGAGATGTTTATGAAGAG | |
| | GATTCCTACGTGAAGAGGAGCCAGGGTCGGATTCCAGTTAAATGGATGGCAA | |
| | TTGAATCCCTTTTTGATCATATCTACACCACGCAAAGTGATGTATGGTCTTT | |
| | TGGTGTCCTGCTGTGGGAGATCGTGACCCTAGGGGGAAACCCCTATCCTGGG | |
| | ATTCCTCCTGAGCGGCTCTTCAACCTTCTGAAGACCGGCCACCGGATGGAGA | |
| | GGCCAGACAACTGCAGCGAGGAGATGTACCGCCTGATGCTGCAATGCTGGAA | |
| | GCAGGAGCCGGACAAAAGGCCGGTGTTTGCGGACATCAGCAAAGACCTGGAG | |
| | AAGATGATGGTTAAGAGGAGAGACTACTTGGACCTTGCGGCGTCCACTCCAT | |
| | CTGACTCCCTGATTTATGACGACGGCCTCTCAGAGGAGGAGACACCGCTGGT | |
| | GGACTGTAATAATGCCCCCCTCCCTCGAGCCCTCCCTTCCACATGGATTGAA | |
| | AACAAACTCTATGGTAGAATTTCCCATGCATTTACTAGATTCTAG | |
| PRKG1- | ATGGGCACCTTGCGGGATTTACAGTACGCGCTCCAGGAGAAGATCGAGGAGC | 53 |
| RET | TGAGGCAGCGGGATGCTCTCATCGACGAGCTGGAGCTGGAGTTGGATCAGAA | |
| | GGACGAACTGATCCAGAAGCTGCAGAACGAGCTGGACAAGTACCGCTCGGTG | |
| | ATCCGACCAGCCACCCAGCAGGCGCAGAAGCAGAGCGCGAGCACCTTGCAGG | |
| | GCGAGCCGCGCACCAAGCGGCAGGCGATCTCCGCCGAGCCCACCGCCTTCGA | |
| | CATCCAGGATCTCAGCCATGTGACCCTGCCCTTCTACCCCAAGAGCCCACAG | |
| | TCCAAGGATCTTATAAAGGAAGCTATCCTTGACAATGACTTTATGAAGAACT | |
| | TGGAGCTGTCGCAGATCCAGGAGATTGTGGATTGTATGTACCCGGTGGAGTA | |
| | TGGCAAGGACAGTTGCATCATCAAAGAAGGAGACGTGGGGTCACTGGTGTAT | |
| | GTCATGGAAGATGGTAAGGTTGAAGTTACAAAAGAAGGTGTGAAGTTGTGTA | |
| | CCATGGGTCCAGGAAAAGTGTTTGGGGAATTGGCTATTCTTTACAACTGTAC | |
| | CCGGACAGCGACCGTCAAGACTCTTGTAAATGTAAAACTCTGGGCCATTGAT | |
| | CGACAATGTTTTCAAACAATAATGATGAGGACAGGACTCATCAAGCATACCG | |
| | AGTATATGGAATTTTTAAAAAGCGTTCCAACATTCCAGAGCCTTCCTGAAGA | |
| | GATCCTCAGCAAGCTTGCTGATGTCCTTGAAGAGGTACCGCCTGATGCTGCA | |
| | ATGCTGGAAGCAGGAGCCGGACAAAAGGCCGGTGTTTGCGGACATCAGCAAA | |
| | GACCTGGAGAAGATGATGGTTAAGAGGAGAGACTACTTGGACCTTGCGGCGT | |
| | CCACTCCATCTGACTCCCTGATTTATGACGACGGCCTCTCAGAGGAGGAGAC | |
| | ACCGCTGGTGGACTGTAATAATGCCCCCCTCCCTCGAGCCCTCCCTTCCACA | |
| | TGGATTGAAAACAAACTCTATGGTAGAATTTCCCATGCATTTACTAGATTCT | |
| | AG | |
| RUFY2- | ATGACTTTTCAGGTTTGGGGGTGGCGGAGGGAAGACGCATCTCAGGTTTTGG | 54 |
| RET | CCTGGGTGCCTGACGCCGAGGGCGGGAGACGGGGAATGCTGACGCGCAGGAG | |
| | CCTGGCTACAAAAGACCCCACAGCTGTAGAGAGAGCAAACTTGTTAAACATG | |
| | GCTAAACTGAGTATCAAAGGACTCATTGAATCTGCTCTGAGCTTTGGCCGCA | |
| | CTTTGGATTCTGACTATCCCCCCTTGCAGCAATTCTTTGTTGTTATGGAACA | |
| | TTGCCTGAAACACGGTCTTAAAGTAAGAAAATCATTTTTGAGTTACAACAAA | |
| | ACCATCTGGGGCCCTTTGGAACTGGTGGAGAAGCTGTACCCCGAAGCAGAGG | |
| | AAATAGGAGCTAGTGTCCGGGATCTACCTGGTCTGAAGACCCCTCTGGGTCG | |
| | AGCAAGAGCGTGGCTTCGATTAGCCCTCATGCAAAAAAAAATGGCCGATTAC | |
| | TTACGTTGCTTAATTATTCAGAGGGATCTCTTGAGTGAGTTTTATGAGTATC | |
| | ACGCACTAATGATGGAAGAAGAAGGAGCAGTAATTGTTGGGCTGCTGGTTGG | |
| | CCTGAATGTGATCGATGCTAATCTGTGTGTGAAGGGAGAGGATTTAGACTCA | |
| | CAAGTTGGAGTGATTGATTTTTCTATGTATTTAAAGAATGAAGAAGATATTG | |
| | GAAATAAAGAAAGGAATGTTCAAATTGCTGCCATATTAGACCAAAAGAATTA | |
| | TGTTGAAGAATTAAATAGACAACTGAACAGCACAGTCAGCAGCCTCCATTCA | |
| | AGAGTTGATTCATTAGAAAAGTCAAATACTAAGCTGATTGAAGAGTTAGCAA | |
| | TAGCAAAGAATAACATCATTAAACTCCAGGAAGAAAATCATCAATTACGAAG | |
| | TGAAAATAAATTGATTTTAATGAAAACACAGCAGCACCTAGAGGAGGATCCA | |
| | AAGTGGGAATTCCCTCGGAAGAACTTGGTTCTTGGAAAAACTCTAGGAGAAG | |
| | GCGAATTTGGAAAAGTGGTCAAGGCAACGGCCTTCCATCTGAAAGGCAGAGC | |
| | AGGGTACACCACGGTGGCCGTGAAGATGCTGAAAGAGAACGCCTCCCCGAGT | |
| | GAGCTGCGAGACCTGCTGTCAGAGTTCAACGTCCTGAAGCAGGTCAACCACC | |
| | CACATGTCATCAAATTGTATGGGGCCTGCAGCCAGGATGGCCCGCTCCTCCT | |
| | CATCGTGGAGTACGCCAAATACGGCTCCCTGCGGGGCTTCCTCCGCGAGAGC | |
| | CGCAAAGTGGGGCCTGGCTACCTGGGCAGTGGAGGCAGCCGCAACTCCAGCT | |
| | CCCTGGACCACCCGGATGAGCGGGCCCTCACCATGGGCGACCTCATCTCATT | |
| | TGCCTGGCAGATCTCACAGGGGATGCAGTATCTGGCCGAGATGAAGCTCGTT | |
| | CATCGGGACTTGGCAGCCAGAAACATCCTGGTAGCTGAGGGGCGGAAGATGA | |
| | AGATTTCGGATTTCGGCTTGTCCCGAGATGTTTATGAAGAGGATTCCTACGT | |
| | GAAGAGGAGCCAGGGTCGGATTCCAGTTAAATGGATGGCAATTGAATCCCTT | |
| | TTTGATCATATCTACACCACGCAAAGTGATGTATGGTCTTTTGGTGTCCTGC | |
| | TGTGGGAGATCGTGACCCTAGGGGGAAACCCCTATCCTGGGATTCCTCCTGA | |
| | GCGGCTCTTCAACCTTCTGAAGACCGGCCACCGGATGGAGAGGCCAGACAAC | |
| | TGCAGCGAGGAGATGTACCGCCTGATGCTGCAATGCTGGAAGCAGGAGCCGG | |
| | ACAAAAGGCCGGTGTTTGCGGACATCAGCAAAGACCTGGAGAAGATGATGGT | |
| | TAAGAGGAGAGACTACTTGGACCTTGCGGCGTCCACTCCATCTGACTCCCTG | |
| | ATTTATGACGACGGCCTCTCAGAGGAGGAGACACCGCTGGTGGACTGTAATA | |
| | ATGCCCCCCTCCCTCGAGCCCTCCCTTCCACATGGATTGAAAACAAACTCTA | |
| | TGGTAGAATTTCCCATGCATTTACTAGATTCTAG | |
| SNRNP70- | ATGACCCAGTTCCTGCCGCCCAACCTTCTGGCCCTCTTTGCCCCCCGTGACC | 55 |
| RET | CTATTCCATACCTGCCACCCCTGGAGAAACTGCCACATGAAAAACACCACAA | |
| | TCAACCTTATTGTGGCATTGCGCCGTACATTCGAGAGTTTGAGGACCCTCGA | |
| | GATGCCCCTCCTCCAACTCGTGCTGAAACCCGAGAGGAGCGCATGGAGAGGA | |
| | AAAGACGGGAAAAGATTGAGCGGCGACAGCAAGAAGTGGAGACAGAGCTTAA | |
| | AATGTGGGACCCTCACAATGATCCCAATGCTCAGGGGGATGCCTTCAAGACT | |
| | CTCTTCGTGGCGAGAGTGAATTATGACACAACAGAATCCAAGCTCCGGAGAG | |
| | AGTTTGAGGTGTACGGACCTATCAAAAGAGAGGATCCAAAGTGGGAATTCCC | |
| | TCGGAAGAACTTGGTTCTTGGAAAAACTCTAGGAGAAGGCGAATTTGGAAAA | |
| | GTGGTCAAGGCAACGGCCTTCCATCTGAAAGGCAGAGCAGGGTACACCACGG | |
| | TGGCCGTGAAGATGCTGAAAGAGAACGCCTCCCCGAGTGAGCTGCGAGACCT | |
| | GCTGTCAGAGTTCAACGTCCTGAAGCAGGTCAACCACCCACATGTCATCAAA | |
| | TTGTATGGGGCCTGCAGCCAGGATGGCCCGCTCCTCCTCATCGTGGAGTACG | |
| | CCAAATACGGCTCCCTGCGGGGCTTCCTCCGCGAGAGCCGCAAAGTGGGGCC | |
| | TGGCTACCTGGGCAGTGGAGGCAGCCGCAACTCCAGCTCCCTGGACCACCCG | |
| | GATGAGCGGGCCCTCACCATGGGCGACCTCATCTCATTTGCCTGGCAGATCT | |
| | CACAGGGGATGCAGTATCTGGCCGAGATGAAGCTCGTTCATCGGGACTTGGC | |
| | AGCCAGAAACATCCTGGTAGCTGAGGGGCGGAAGATGAAGATTTCGGATTTC | |
| | GGCTTGTCCCGAGATGTTTATGAAGAGGATTCCTACGTGAAGAGGAGCCAGG | |
| | GTCGGATTCCAGTTAAATGGATGGCAATTGAATCCCTTTTTGATCATATCTA | |
| | CACCACGCAAAGTGATGTATGGTCTTTTGGTGTCCTGCTGTGGGAGATCGTG | |
| | ACCCTAGGGGGAAACCCCTATCCTGGGATTCCTCCTGAGCGGCTCTTCAACC | |
| | TTCTGAAGACCGGCCACCGGATGGAGAGGCCAGACAACTGCAGCGAGGAGAT | |
| | GTACCGCCTGATGCTGCAATGCTGGAAGCAGGAGCCGGACAAAAGGCCGGTG | |
| | TTTGCGGACATCAGCAAAGACCTGGAGAAGATGATGGTTAAGAGGAGAGACT | |
| | ACTTGGACCTTGCGGCGTCCACTCCATCTGACTCCCTGATTTATGACGACGG | |
| | CCTCTCAGAGGAGGAGACACCGCTGGTGGACTGTAATAATGCCCCCCTCCCT | |
| | CGAGCCCTCCCTTCCACATGGATTGAAAACAAACTCTATGGTAGAATTTCCC | |
| | ATGCATTTACTAGATTCTAG | |
| SQSTM1- | ATGGCGTCGCTCACCGTGAAGGCCTACCTTCTGGGCAAGGAGGACGCGGCGC | 56 |
| RET | GCGAGATTCGCCGCTTCAGCTTCTGCTGCAGCCCCGAGCCTGAGGCGGAAGC | |
| | CGAGGCTGCGGCGGGTCCGGGACCCTGCGAGCGGCTGCTGAGCCGGGTGGCC | |
| | GCCCTGTTCCCCGCGCTGCGGCCTGGCGGCTTCCAGGCGCACTACCGCGATG | |
| | AGGACGGGGACTTGGTTGCCTTTTCCAGTGACGAGGAATTGACAATGGCCAT | |
| | GTCCTACGTGAAGGATGACATCTTCCGAATCTACATTAAAGAGAAAAAAGAG | |
| | TGCCGGCGGGACCACCGCCCACCGTGTGCTCAGGAGGCGCCCCGCAACATGG | |
| | TGCACCCCAATGTGATCTGCGATGGCTGCAATGGGCCTGTGGTAGGAACCCG | |
| | CTACAAGTGCAGCGTCTGCCCAGACTACGACTTGTGTAGCGTCTGCGAGGGA | |
| | AAGGGCTTGCACCGGGGGCACACCAAGCTCGCATTCCCCAGCCCCTTCGGGC | |
| | ACCTGTCTGAGGGCTTCTCGCACAGCCGCTGGCTCCGGAAGGTGAAACACGG | |
| | ACACTTCGGGTGGCCAGGATGGGAAATGGGTCCACCAGGAAACTGGAGCCCA | |
| | CGTCCTCCTCGTGCAGGGGAGGCCCGCCCTGGCCCCACGGCAGAATCAGCTT | |
| | CTGGTCCATCGGAGGATCCGAGTGTGAATTTCCTGAAGAACGTTGGGGAGAG | |
| | TGTGGCAGCTGCCCTTAGCCCTCTGGATCCACTGTGCGACGAGCTGTGCCGC | |
| | ACGGTGATCGCAGCCGCTGTCCTCTTCTCCTTCATCGTCTCGGTGCTGCTGT | |
| | CTGCCTTCTGCATCCACTGCTACCACAAGTTTGCCCACAAGCCACCCATCTC | |
| | CTCAGCTGAGATGACCTTCCGGAGGCCCGCCCAGGCCTTCCCGGTCAGCTAC | |
| | TCCTCTTCCGGTGCCCGCCGGCCCTCGCTGGACTCCATGGAGAACCAGGTCT | |
| | CCGTGGATGCCTTCAAGATCCTGGAGGATCCAAAGTGGGAATTCCCTCGGAA | |
| | GAACTTGGTTCTTGGAAAAACTCTAGGAGAAGGCGAATTTGGAAAAGTGGTC | |
| | AAGGCAACGGCCTTCCATCTGAAAGGCAGAGCAGGGTACACCACGGTGGCCG | |
| | TGAAGATGCTGAAAGAGAACGCCTCCCCGAGTGAGCTGCGAGACCTGCTGTC | |
| | AGAGTTCAACGTCCTGAAGCAGGTCAACCACCCACATGTCATCAAATTGTAT | |
| | GGGGCCTGCAGCCAGGATGGCCCGCTCCTCCTCATCGTGGAGTACGCCAAAT | |
| | ACGGCTCCCTGCGGGGCTTCCTCCGCGAGAGCCGCAAAGTGGGGCCTGGCTA | |
| | CCTGGGCAGTGGAGGCAGCCGCAACTCCAGCTCCCTGGACCACCCGGATGAG | |
| | CGGGCCCTCACCATGGGCGACCTCATCTCATTTGCCTGGCAGATCTCACAGG | |
| | GGATGCAGTATCTGGCCGAGATGAAGCTCGTTCATCGGGACTTGGCAGCCAG | |
| | AAACATCCTGGTAGCTGAGGGGCGGAAGATGAAGATTTCGGATTTCGGCTTG | |
| | TCCCGAGATGTTTATGAAGAGGATTCCTACGTGAAGAGGAGCCAGGGTCGGA | |
| | TTCCAGTTAAATGGATGGCAATTGAATCCCTTTTTGATCATATCTACACCAC | |
| | GCAAAGTGATGTATGGTCTTTTGGTGTCCTGCTGTGGGAGATCGTGACCCTA | |
| | GGGGGAAACCCCTATCCTGGGATTCCTCCTGAGCGGCTCTTCAACCTTCTGA | |
| | AGACCGGCCACCGGATGGAGAGGCCAGACAACTGCAGCGAGGAGATGTACCG | |
| | CCTGATGCTGCAATGCTGGAAGCAGGAGCCGGACAAAAGGCCGGTGTTTGCG | |
| | GACATCAGCAAAGACCTGGAGAAGATGATGGTTAAGAGGAGAGACTACTTGG | |
| | ACCTTGCGGCGTCCACTCCATCTGACTCCCTGATTTATGACGACGGCCTCTC | |
| | AGAGGAGGAGACACCGCTGGTGGACTGTAATAATGCCCCCCTCCCTCGAGCC | |
| | CTCCCTTCCACATGGATTGAAAACAAACTCTATGGTAGAATTTCCCATGCAT | |
| | TTACTAGATTCTAG | |
| TNIP1- | ATGGAAGGGAGAGGACCGTACCGGATCTACGACCCTGGGGGCAGCGTGCCCT | 57 |
| RET | CAGGAGAGGCATCCGCAGCTTTTGAGCGCCTAGTGAAGGAGAATTCCCGGCT | |
| | GAAGGAAAAAATGCAAGGGATAAAGATGTTAGGGGAGCTTTTGGAAGAGTCC | |
| | CAGATGGAAGCGACCAGGCTCCGGCAGAAGGCAGAGGAGCTAGTGAAGGACA | |
| | ACGAGCTGCTCCCACCACCTTCTCCCTCCTTGGGCTCCTTCGACCCCCTGGC | |
| | TGAGCTCACAGGAAAGGACTCAAATGTCACAGCATCTCCCACAGCCCCTGCA | |
| | TGCCCCAGTGACAAGCCAGCACCAGTCCAGAAGCCTCCATCCAGTGGCACCT | |
| | CCTCTGAATTTGAAGTGGTCACTCCTGAGGAGCAGAATTCACCAGAGAGCAG | |
| | CAGCCATGCCAATGCGATGGCGCTGGGCCCCCTGCCCCGTGAGGACGGCAAC | |
| | CTGATGCTGCACCTGCAGCGCCTGGAGACCACGCTGAGTGTGTGTGCCGAGG | |
| | AGCCGGACCACGGCCAGCTCTTCACCCACCTGGGCCGCATGGCCCTGGAGTT | |
| | CAACCGACTGGCATCCAAGGTGCACAAGAATGAGCAGCGCACCTCCATTCTG | |
| | CAGACCCTGTGTGAGCAGCTTCGGAAGGAGAACGAGGCTCTGAAGGCCAAGT | |
| | TGGATAAGGGCCTGGAACAGCGGGATCAGGCTGCCGAGAGGCTGCGGGAGGA | |
| | AAATTTGGAGCTCAAGAAGTTGTTGATGAGCAATGGCAACAAAGAGGGTGCG | |
| | TCTGGGCGGCCAGGCTCACCGAAGATGGAAGGGACAGGCAAGAAGGCAGTGG | |
| | CTGGACAGCAGCAGGCTAGTGTGACGGCAGGTAAGGTCCCAGAGGTGGTGGC | |
| | CTTGGGCGCAGCCGAGAAGAAGGTGAAGATGCTGGAGCAGCAGCGCAGTGAG | |
| | CTGCTGGAAGTGAACAAGCAGTGGGACCAGCATTTCCGGTCCATGAAGCAGC | |
| | AGTATGAGCAGAAGATCACTGAGCTGCGTCAGAAGCTGGCTGATTTGCAGAA | |
| | GCAGGTGACTGACCTGGAGGCCGAGCGGGAGCAGAAGCAGCGTGACTTTGAC | |
| | CGCAAGCTCCTCCTGGCCAAGTCCAAGATTGAAATGGAGGAGACCGACAAGG | |
| | AGCAGCTGACAGCAGAGGCCAAGGAGCTGCGCCAAAAGGTCAAGTACCTGCA | |
| | GGATCAGCTGAGCCCACTCACCCGACAGCGTGAGTACCAGGAAAAGGAGATC | |
| | CAGCGGCTCAACAAGGCCCTGGAGGAAGCACTGAGCATCCAAACCCCGCCAT | |
| | CATCTCCACCAACAGCATTTGGGAGCCCAGAAGGAGCAGGGGCCCTCCTAAG | |
| | GAAACAGGAGCTGGTCACGCAGAATGAGTTGCTGAAACAGCAGGAGGATCCA | |
| | AAGTGGGAATTCCCTCGGAAGAACTTGGTTCTTGGAAAAACTCTAGGAGAAG | |
| | GCGAATTTGGAAAAGTGGTCAAGGCAACGGCCTTCCATCTGAAAGGCAGAGC | |
| | AGGGTACACCACGGTGGCCGTGAAGATGCTGAAAGAGAACGCCTCCCCGAGT | |
| | GAGCTGCGAGACCTGCTGTCAGAGTTCAACGTCCTGAAGCAGGTCAACCACC | |
| | CACATGTCATCAAATTGTATGGGGCCTGCAGCCAGGATGGCCCGCTCCTCCT | |
| | CATCGTGGAGTACGCCAAATACGGCTCCCTGCGGGGCTTCCTCCGCGAGAGC | |
| | CGCAAAGTGGGGCCTGGCTACCTGGGCAGTGGAGGCAGCCGCAACTCCAGCT | |
| | CCCTGGACCACCCGGATGAGCGGGCCCTCACCATGGGCGACCTCATCTCATT | |
| | TGCCTGGCAGATCTCACAGGGGATGCAGTATCTGGCCGAGATGAAGCTCGTT | |
| | CATCGGGACTTGGCAGCCAGAAACATCCTGGTAGCTGAGGGGCGGAAGATGA | |
| | AGATTTCGGATTTCGGCTTGTCCCGAGATGTTTATGAAGAGGATTCCTACGT | |
| | GAAGAGGAGCCAGGGTCGGATTCCAGTTAAATGGATGGCAATTGAATCCCTT | |
| | TTTGATCATATCTACACCACGCAAAGTGATGTATGGTCTTTTGGTGTCCTGC | |
| | TGTGGGAGATCGTGACCCTAGGGGGAAACCCCTATCCTGGGATTCCTCCTGA | |
| | GCGGCTCTTCAACCTTCTGAAGACCGGCCACCGGATGGAGAGGCCAGACAAC | |
| | TGCAGCGAGGAGATGTACCGCCTGATGCTGCAATGCTGGAAGCAGGAGCCGG | |
| | ACAAAAGGCCGGTGTTTGCGGACATCAGCAAAGACCTGGAGAAGATGATGGT | |
| | TAAGAGGAGAGACTACTTGGACCTTGCGGCGTCCACTCCATCTGACTCCCTG | |
| | ATTTATGACGACGGCCTCTCAGAGGAGGAGACACCGCTGGTGGACTGTAATA | |
| | ATGCCCCCCTCCCTCGAGCCCTCCCTTCCACATGGATTGAAAACAAACTCTA | |
| | TGGTAGAATTTCCCATGCATTTACTAGATTCTAG | |
| TNIP1- | ATGGAAGGGAGAGGACCGTACCGGATCTACGACCCTGGGGGCAGCGTGCCCT | 58 |
| RET | CAGGAGAGGCATCCGCAGCTTTTGAGCGCCTAGTGAAGGAGAATTCCCGGCT | |
| | GAAGGAAAAAATGCAAGGGATAAAGATGTTAGGGGAGCTTTTGGAAGAGTCC | |
| | CAGATGGAAGCGACCAGGCTCCGGCAGAAGGCAGAGGAGCTAGTGAAGGACA | |
| | ACGAGCTGCTCCCACCACCTTCTCCCTCCTTGGGCTCCTTCGACCCCCTGGC | |
| | TGAGCTCACAGGAAAGGACTCAAATGTCACAGCATCTCCCACAGCCCCTGCA | |
| | TGCCCCAGTGACAAGCCAGCACCAGTCCAGAAGCCTCCATCCAGTGGCACCT | |
| | CCTCTGAATTTGAAGTGGTCACTCCTGAGGAGCAGAATTCACCAGAGAGCAG | |
| | CAGCCATGCCAATGCGATGGCGCTGGGCCCCCTGCCCCGTGAGGACGGCAAC | |
| | CTGATGCTGCACCTGCAGCGCCTGGAGACCACGCTGAGTGTGTGTGCCGAGG | |
| | AGCCGGACCACGGCCAGCTCTTCACCCACCTGGGCCGCATGGCCCTGGAGTT | |
| | CAACCGACTGGCATCCAAGGTGCACAAGAATGAGCAGCGCACCTCCATTCTG | |
| | CAGACCCTGTGTGAGCAGCTTCGGAAGGAGAACGAGGCTCTGAAGGCCAAGT | |
| | TGGATAAGGGCCTGGAACAGCGGGATCAGGCTGCCGAGAGGCTGCGGGAGGA | |
| | AAATTTGGAGCTCAAGAAGTTGTTGATGAGCAATGGCAACAAAGAGGGTGCG | |
| | TCTGGGCGGCCAGGCTCACCGAAGATGGAAGGGACAGGCAAGAAGGCAGTGG | |
| | CTGGACAGCAGCAGGCTAGTGTGACGGCAGGTAAGGTCCCAGAGGTGGTGGC | |
| | CTTGGGCGCAGCCGAGAAGAAGGTGAAGATGCTGGAGCAGCAGCGCAGTGAG | |
| | CTGCTGGAAGTGAACAAGCAGTGGGACCAGCATTTCCGGTCCATGAAGCAGC | |
| | AGTATGAGCAGAAGATCACTGAGCTGCGTCAGAAGCTGGCTGATTTGCAGAA | |
| | GCAGGTGACTGACCTGGAGGCCGAGCGGGAGCAGAAGCAGCGTGACTTTGAC | |
| | CGCAAGCTCCTCCTGGCCAAGTCCAAGATTGAAATGGAGGAGACCGACAAGG | |
| | AGCAGCTGACAGCAGAGGCCAAGGAGCTGCGCCAAAAGGTCAAGTACCTGCA | |
| | GGATCAGCTGAGCCCACTCACCCGACAGCGTGAGTACCAGGAAAAGGAGATC | |
| | CAGCGGCTCAACAAGGCCCTGGAGGAAGCACTGAGCATCCAAACCCCGCCAT | |
| | CATCTCCACCAACAGCATTTGGGAGCCCAGAAGGAGCAGGGGCCCTCCTAAG | |
| | GAAACAGGAGCTGGTCACGCAGAATGAGTTGCTGAAACAGCAGGTGAAGATC | |
| | TTCGAGGAGGACTTCCAGAGGGAGCGCAGTGATCGTGAGCGCATGAATGAGG | |
| | AGAAGGAAGAGCTGAAGAAGCAAGTGGAGAAGCTGCAGGCCCAGGTCACCCT | |
| | GTCAAATGCCCAGCTAAAAGCATTCAAAGATGAGGAGAAGGCAAGAGAAGCC | |
| | CTCAGACAGCAGAAGAGGAAAGCAAAGGCCTCAGGAGAGCGTTACCATGTGG | |
| | AGCCCCACCCAGAACATCTCTGCGGGGCCTACCCCTACGCCTACCCGCCCAT | |
| | GCCAGCCATGGTGCCACACCATGGCTTCGAGGACTGGTCCCAGATCCGCTAC | |
| | CCCCCTCCCCCCATGGCCATGGAGCACCCGCCCCCACTCCCCAACTCGCGCC | |
| | TCTTCCATCTGGAGGATCCAAAGTGGGAATTCCCTCGGAAGAACTTGGTTCT | |
| | TGGAAAAACTCTAGGAGAAGGCGAATTTGGAAAAGTGGTCAAGGCAACGGCC | |
| | TTCCATCTGAAAGGCAGAGCAGGGTACACCACGGTGGCCGTGAAGATGCTGA | |
| | AAGAGAACGCCTCCCCGAGTGAGCTGCGAGACCTGCTGTCAGAGTTCAACGT | |
| | CCTGAAGCAGGTCAACCACCCACATGTCATCAAATTGTATGGGGCCTGCAGC | |
| | CAGGATGGCCCGCTCCTCCTCATCGTGGAGTACGCCAAATACGGCTCCCTGC | |
| | GGGGCTTCCTCCGCGAGAGCCGCAAAGTGGGGCCTGGCTACCTGGGCAGTGG | |
| | AGGCAGCCGCAACTCCAGCTCCCTGGACCACCCGGATGAGCGGGCCCTCACC | |
| | ATGGGCGACCTCATCTCATTTGCCTGGCAGATCTCACAGGGGATGCAGTATC | |
| | TGGCCGAGATGAAGCTCGTTCATCGGGACTTGGCAGCCAGAAACATCCTGGT | |
| | AGCTGAGGGGCGGAAGATGAAGATTTCGGATTTCGGCTTGTCCCGAGATGTT | |
| | TATGAAGAGGATTCCTACGTGAAGAGGAGCCAGGGTCGGATTCCAGTTAAAT | |
| | GGATGGCAATTGAATCCCTTTTTGATCATATCTACACCACGCAAAGTGATGT | |
| | ATGGTCTTTTGGTGTCCTGCTGTGGGAGATCGTGACCCTAGGGGGAAACCCC | |
| | TATCCTGGGATTCCTCCTGAGCGGCTCTTCAACCTTCTGAAGACCGGCCACC | |
| | GGATGGAGAGGCCAGACAACTGCAGCGAGGAGATGTACCGCCTGATGCTGCA | |
| | ATGCTGGAAGCAGGAGCCGGACAAAAGGCCGGTGTTTGCGGACATCAGCAAA | |
| | GACCTGGAGAAGATGATGGTTAAGAGGAGAGACTACTTGGACCTTGCGGCGT | |
| | CCACTCCATCTGACTCCCTGATTTATGACGACGGCCTCTCAGAGGAGGAGAC | |
| | ACCGCTGGTGGACTGTAATAATGCCCCCCTCCCTCGAGCCCTCCCTTCCACA | |
| | TGGATTGAAAACAAACTCTATGGTAGAATTTCCCATGCATTTACTAGATTCT | |
| | AG | |
| TNIP1- | ATGGAAGGGAGAGGACCGTACCGGATCTACGACCCTGGGGGCAGCGTGCCCT | 59 |
| RET | CAGGAGAGGCATCCGCAGCTTTTGAGCGCCTAGTGAAGGAGAATTCCCGGCT | |
| | GAAGGAAAAAATGCAAGGGATAAAGATGTTAGGGGAGCTTTTGGAAGAGTCC | |
| | CAGATGGAAGCGACCAGGCTCCGGCAGAAGGCAGAGGAGCTAGTGAAGGACA | |
| | ACGAGCTGCTCCCACCACCTTCTCCCTCCTTGGGCTCCTTCGACCCCCTGGC | |
| | TGAGCTCACAGGAAAGGACTCAAATGTCACAGCATCTCCCACAGCCCCTGCA | |
| | TGCCCCAGTGACAAGCCAGCACCAGTCCAGAAGCCTCCATCCAGTGGCACCT | |
| | CCTCTGAATTTGAAGTGGTCACTCCTGAGGAGCAGAATTCACCAGAGAGCAG | |
| | CAGCCATGCCAATGCGATGGCGCTGGGCCCCCTGCCCCGTGAGGACGGCAAC | |
| | CTGATGCTGCACCTGCAGCGCCTGGAGACCACGCTGAGTGTGTGTGCCGAGG | |
| | AGCCGGACCACGGCCAGCTCTTCACCCACCTGGGCCGCATGGCCCTGGAGTT | |
| | CAACCGACTGGCATCCAAGGTGCACAAGAATGAGCAGCGCACCTCCATTCTG | |
| | CAGACCCTGTGTGAGCAGCTTCGGAAGGAGAACGAGGCTCTGAAGGCCAAGT | |
| | TGGATAAGGGCCTGGAACAGCGGGATCAGGCTGCCGAGAGGCTGCGGGAGGA | |
| | AAATTTGGAGCTCAAGAAGTTGTTGATGAGCAATGGCAACAAAGAGGGTGCG | |
| | TCTGGGCGGCCAGGCTCACCGAAGATGGAAGGGACAGGCAAGAAGGCAGTGG | |
| | CTGGACAGCAGCAGGCTAGTGTGACGGCAGGTAAGGTCCCAGAGGTGGTGGC | |
| | CTTGGGCGCAGCCGAGAAGAAGGTGAAGATGCTGGAGCAGCAGCGCAGTGAG | |
| | CTGCTGGAAGTGAACAAGCAGTGGGACCAGCATTTCCGGTCCATGAAGCAGC | |
| | AGTATGAGCAGAAGATCACTGAGCTGCGTCAGAAGCTGGCTGATTTGCAGAA | |
| | GCAGGTGACTGACCTGGAGGCCGAGCGGGAGCAGAAGCAGCGTGACTTTGAC | |
| | CGCAAGCTCCTCCTGGCCAAGTCCAAGATTGAAATGGAGGAGACCGACAAGG | |
| | AGCAGCTGACAGCAGAGGCCAAGGAGCTGCGCCAAAAGGTCAAGTACCTGCA | |
| | GGATCAGCTGAGCCCACTCACCCGACAGCGTGAGTACCAGGAAAAGGAGATC | |
| | CAGCGGCTCAACAAGGAGGATCCAAAGTGGGAATTCCCTCGGAAGAACTTGG | |
| | TTCTTGGAAAAACTCTAGGAGAAGGCGAATTTGGAAAAGTGGTCAAGGCAAC | |
| | GGCCTTCCATCTGAAAGGCAGAGCAGGGTACACCACGGTGGCCGTGAAGATG | |
| | CTGAAAGAGAACGCCTCCCCGAGTGAGCTGCGAGACCTGCTGTCAGAGTTCA | |
| | ACGTCCTGAAGCAGGTCAACCACCCACATGTCATCAAATTGTATGGGGCCTG | |
| | CAGCCAGGATGGCCCGCTCCTCCTCATCGTGGAGTACGCCAAATACGGCTCC | |
| | CTGCGGGGCTTCCTCCGCGAGAGCCGCAAAGTGGGGCCTGGCTACCTGGGCA | |
| | GTGGAGGCAGCCGCAACTCCAGCTCCCTGGACCACCCGGATGAGCGGGCCCT | |
| | CACCATGGGCGACCTCATCTCATTTGCCTGGCAGATCTCACAGGGGATGCAG | |
| | TATCTGGCCGAGATGAAGCTCGTTCATCGGGACTTGGCAGCCAGAAACATCC | |
| | TGGTAGCTGAGGGGCGGAAGATGAAGATTTCGGATTTCGGCTTGTCCCGAGA | |
| | TGTTTATGAAGAGGATTCCTACGTGAAGAGGAGCCAGGGTCGGATTCCAGTT | |
| | AAATGGATGGCAATTGAATCCCTTTTTGATCATATCTACACCACGCAAAGTG | |
| | ATGTATGGTCTTTTGGTGTCCTGCTGTGGGAGATCGTGACCCTAGGGGGAAA | |
| | CCCCTATCCTGGGATTCCTCCTGAGCGGCTCTTCAACCTTCTGAAGACCGGC | |
| | CACCGGATGGAGAGGCCAGACAACTGCAGCGAGGAGATGTACCGCCTGATGC | |
| | TGCAATGCTGGAAGCAGGAGCCGGACAAAAGGCCGGTGTTTGCGGACATCAG | |
| | CAAAGACCTGGAGAAGATGATGGTTAAGAGGAGAGACTACTTGGACCTTGCG | |
| | GCGTCCACTCCATCTGACTCCCTGATTTATGACGACGGCCTCTCAGAGGAGG | |
| | AGACACCGCTGGTGGACTGTAATAATGCCCCCCTCCCTCGAGCCCTCCCTTC | |
| | CACATGGATTGAAAACAAACTCTATGGTAGAATTTCCCATGCATTTACTAGA | |
| | TTCTAG | |
| TRIM27- | ATGGCCTCCGGGAGTGTGGCCGAGTGCCTGCAGCAGGAGACCACCTGCCCCG | 60 |
| RET | TGTGCCTGCAGTACTTCGCAGAGCCCATGATGCTCGACTGCGGCCATAACAT | |
| | CTGTTGCGCGTGCCTCGCCCGCTGCTGGGGCACGGCAGAGACTAACGTGTCG | |
| | TGCCCGCAGTGCCGGGAGACCTTCCCGCAGAGGCACATGCGGCCCAACCGGC | |
| | ACCTGGCCAACGTGACCCAACTGGTAAAGCAGCTGCGCACCGAGCGGCCGTC | |
| | GGGGCCCGGCGGCGAGATGGGCGTGTGCGAGAAGCACCGCGAGCCCCTGAAG | |
| | CTGTACTGCGAGGAGGACCAGATGCCCATCTGCGTGGTGTGCGACCGCTCCC | |
| | GCGAGCACCGCGGCCACAGCGTGCTGCCGCTCGAGGAGGCGGTGGAGGGCTT | |
| | CAAGGAGCAAATCCAGAACCAGCTCGACCATTTAAAAAGAGTGAAAGATTTA | |
| | AAGAAGAGACGTCGGGCCCAGGGGGAACAGGCACGAGCTGAACTCTTGAGCC | |
| | TAACCCAGATGGAGAGGGAGAAGATTGTTTGGGAGTTTGAGCAGCTGTATCA | |
| | CTCCTTAAAGGAGCATGAGTATCGCCTCCTGGCCCGCCTTGAGGAGCTAGAC | |
| | TTGGCCATCTACAATAGCATCAATGGTGCCATCACCCAGTTCTCTTGCAACA | |
| | TCTCCCACCTCAGCAGCCTGATCGCTCAGCTAGAAGAGAAGCAGCAGCAGCC | |
| | CACCAGGGAGCTCCTGCAGGAGGATCCAAAGTGGGAATTCCCTCGGAAGAAC | |
| | TTGGTTCTTGGAAAAACTCTAGGAGAAGGCGAATTTGGAAAAGTGGTCAAGG | |
| | CAACGGCCTTCCATCTGAAAGGCAGAGCAGGGTACACCACGGTGGCCGTGAA | |
| | GATGCTGAAAGAGAACGCCTCCCCGAGTGAGCTGCGAGACCTGCTGTCAGAG | |
| | TTCAACGTCCTGAAGCAGGTCAACCACCCACATGTCATCAAATTGTATGGGG | |
| | CCTGCAGCCAGGATGGCCCGCTCCTCCTCATCGTGGAGTACGCCAAATACGG | |
| | CTCCCTGCGGGGCTTCCTCCGCGAGAGCCGCAAAGTGGGGCCTGGCTACCTG | |
| | GGCAGTGGAGGCAGCCGCAACTCCAGCTCCCTGGACCACCCGGATGAGCGGG | |
| | CCCTCACCATGGGCGACCTCATCTCATTTGCCTGGCAGATCTCACAGGGGAT | |
| | GCAGTATCTGGCCGAGATGAAGCTCGTTCATCGGGACTTGGCAGCCAGAAAC | |
| | ATCCTGGTAGCTGAGGGGCGGAAGATGAAGATTTCGGATTTCGGCTTGTCCC | |
| | GAGATGTTTATGAAGAGGATTCCTACGTGAAGAGGAGCCAGGGTCGGATTCC | |
| | AGTTAAATGGATGGCAATTGAATCCCTTTTTGATCATATCTACACCACGCAA | |
| | AGTGATGTATGGTCTTTTGGTGTCCTGCTGTGGGAGATCGTGACCCTAGGGG | |
| | GAAACCCCTATCCTGGGATTCCTCCTGAGCGGCTCTTCAACCTTCTGAAGAC | |
| | CGGCCACCGGATGGAGAGGCCAGACAACTGCAGCGAGGAGATGTACCGCCTG | |
| | ATGCTGCAATGCTGGAAGCAGGAGCCGGACAAAAGGCCGGTGTTTGCGGACA | |
| | TCAGCAAAGACCTGGAGAAGATGATGGTTAAGAGGAGAGACTACTTGGACCT | |
| | TGCGGCGTCCACTCCATCTGACTCCCTGATTTATGACGACGGCCTCTCAGAG | |
| | GAGGAGACACCGCTGGTGGACTGTAATAATGCCCCCCTCCCTCGAGCCCTCC | |
| | CTTCCACATGGATTGAAAACAAACTCTATGGTAGAATTTCCCATGCATTTAC | |
| | TAGATTCTAG | |
| TRIM33- | ATGGCGGAAAACAAAGGCGGCGGCGAGGCTGAGAGCGGCGGCGGGGGCAGCG | 61 |
| RET | GCAGCGCGCCGGTAACTGCCGGGGCCGCCGGGCCCGCCGCGCAGGAGGCGGA | |
| | GCCGCCTCTCACCGCGGTGCTGGTGGAGGAGGAGGAGGAGGAAGGCGGCAGG | |
| | GCCGGCGCTGAGGGCGGCGCGGCCGGGCCCGACGACGGGGGGGTGGCCGCGG | |
| | CCTCCTCGGGCTCGGCCCAGGCTGCTTCATCTCCTGCGGCCTCAGTGGGCAC | |
| | TGGAGTTGCCGGGGGCGCAGTATCGACGCCGGCTCCAGCTCCAGCCTCGGCT | |
| | CCCGCTCCGGGTCCCTCGGCAGGGCCGCCTCCTGGACCGCCAGCCTCGCTCC | |
| | TGGACACCTGCGCCGTGTGTCAGCAGAGCTTGCAGAGCCGGCGTGAGGCGGA | |
| | GCCCAAGCTGCTGCCCTGTCTTCACTCCTTCTGCCTGCGCTGCCTGCCCGAG | |
| | CCGGAGCGCCAGCTCAGCGTGCCCATCCCGGGGGGCAGCAACGGCGACATCC | |
| | AGCAAGTTGGTGTAATACGGTGCCCAGTATGCCGCCAAGAATGCAGACAGAT | |
| | AGACCTTGTGGATAATTATTTTGTGAAAGACACATCTGAAGCTCCTAGCAGT | |
| | TCTGATGAAAAATCAGAACAGGTATGTACTAGTTGTGAAGACAATGCAAGTG | |
| | CAGTTGGCTTTTGTGTAGAATGTGGAGAGTGGCTATGTAAGACATGTATCGA | |
| | AGCACATCAAAGAGTAAAATTTACTAAAGATCACTTGATCAGGAAGAAAGAA | |
| | GATGTCTCAGAGTCTGTTGGAGCATCTGGTCAACGCCCTGTTTTCTGCCCTG | |
| | TACACAAACAAGAACAGTTGAAACTTTTCTGTGAAACATGTGATAGATTGAC | |
| | ATGTAGAGACTGTCAGCTATTGGAACACAAAGAACATAGGTATCAGTTTTTG | |
| | GAAGAAGCTTTTCAAAATCAGAAGGGTGCAATTGAGAATCTACTGGCGAAAC | |
| | TTCTTGAGAAGAAGAATTATGTTCATTTTGCAGCTACTCAGGTGCAGAATAG | |
| | GATAAAAGAAGTAAATGAGACTAACAAACGAGTAGAACAGGAAATTAAAGTG | |
| | GCCATTTTCACCCTTATCAATGAAATTAATAAGAAAGGAAAATCTCTCTTAC | |
| | AACAGCTAGAGAATGTTACAAAGGAAAGACAGATGAAGTTACTACAGCAGCA | |
| | GAATGACATCACAGGCCTTTCCCGGCAGGTGAAGCATGTTATGAACTTCACA | |
| | AATTGGGCAATTGCAAGTGGCAGCAGCACAGCACTACTATACAGCAAGCGAC | |
| | TGATTACTTTCCAGTTGCGTCATATTTTGAAAGCACGGTGTGATCCTGTCCC | |
| | TGCTGCTAATGGAGCAATACGTTTCCATTGTGATCCCACCTTCTGGGCAAAG | |
| | AATGTAGTCAATTTAGGTAATCTAGTAATAGAGAGTAAACCAGCTCCTGGTT | |
| | ATACTCCTAATGTTGTAGTTGGGCAAGTTCCTCCAGGGACAAACCACATTAG | |
| | TAAAACCCCTGGACAGATTAACTTAGCACAGCTTCGACTCCAGCACATGCAA | |
| | CAACAAGTATATGCACAGAAACATCAGCAGTTGCAACAGATGAGGATGCAGC | |
| | AACCACCAGCACCTGTACCAACTACAACAACAACAACACAACAGCATCCTAG | |
| | ACAAGCAGCCCCTCAGATGTTACAACAACAGCCTCCTCGATTGATCAGTGTG | |
| | CAAACAATGCAAAGAGGCAACATGAACTGTGGAGCTTTTCAAGCCCATCAGA | |
| | TGAGACTGGCTCAGAATGCTGCCAGAATACCAGGGATACCCAGGCACAGCGG | |
| | CCCTCAATATTCCATGATGCAGCCACACCTCCAAAGACAAGAGGATCCAAAG | |
| | TGGGAATTCCCTCGGAAGAACTTGGTTCTTGGAAAAACTCTAGGAGAAGGCG | |
| | AATTTGGAAAAGTGGTCAAGGCAACGGCCTTCCATCTGAAAGGCAGAGCAGG | |
| | GTACACCACGGTGGCCGTGAAGATGCTGAAAGAGAACGCCTCCCCGAGTGAG | |
| | CTGCGAGACCTGCTGTCAGAGTTCAACGTCCTGAAGCAGGTCAACCACCCAC | |
| | ATGTCATCAAATTGTATGGGGCCTGCAGCCAGGATGGCCCGCTCCTCCTCAT | |
| | CGTGGAGTACGCCAAATACGGCTCCCTGCGGGGCTTCCTCCGCGAGAGCCGC | |
| | AAAGTGGGGCCTGGCTACCTGGGCAGTGGAGGCAGCCGCAACTCCAGCTCCC | |
| | TGGACCACCCGGATGAGCGGGCCCTCACCATGGGCGACCTCATCTCATTTGC | |
| | CTGGCAGATCTCACAGGGGATGCAGTATCTGGCCGAGATGAAGCTCGTTCAT | |
| | CGGGACTTGGCAGCCAGAAACATCCTGGTAGCTGAGGGGCGGAAGATGAAGA | |
| | TTTCGGATTTCGGCTTGTCCCGAGATGTTTATGAAGAGGATTCCTACGTGAA | |
| | GAGGAGCCAGGGTCGGATTCCAGTTAAATGGATGGCAATTGAATCCCTTTTT | |
| | GATCATATCTACACCACGCAAAGTGATGTATGGTCTTTTGGTGTCCTGCTGT | |
| | GGGAGATCGTGACCCTAGGGGGAAACCCCTATCCTGGGATTCCTCCTGAGCG | |
| | GCTCTTCAACCTTCTGAAGACCGGCCACCGGATGGAGAGGCCAGACAACTGC | |
| | AGCGAGGAGATGTACCGCCTGATGCTGCAATGCTGGAAGCAGGAGCCGGACA | |
| | AAAGGCCGGTGTTTGCGGACATCAGCAAAGACCTGGAGAAGATGATGGTTAA | |
| | GAGGAGAGACTACTTGGACCTTGCGGCGTCCACTCCATCTGACTCCCTGATT | |
| | TATGACGACGGCCTCTCAGAGGAGGAGACACCGCTGGTGGACTGTAATAATG | |
| | CCCCCCTCCCTCGAGCCCTCCCTTCCACATGGATTGAAAACAAACTCTATGG | |
| | TAGAATTTCCCATGCATTTACTAGATTCTAG | |
| CCDC6- | ATGGCGGACAGCGCCAGCGAGAGCGACACGGACGGGGCGGGGGGCAACAGCA | 62 |
| RET | GCAGCTCGGCCGCCATGCAGTCGTCCTGCTCGTCGACCTCGGGCGGCGGCGG | |
| | TGGCGGCGGGGGAGGCGGCGGCGGTGGGAAGTCGGGGGGCATTGTCATCTCG | |
| | CCGTTCCGCCTGGAGGAGCTCACCAACCGCCTGGCCTCGCTGCAGCAAGAGA | |
| | ACAAGGTGCTGAAGATAGAGCTGGAGACCTACAAACTGAAGTGCAAGGCACT | |
| | GCAGGAGGAGAACCGCGACCTGCGCAAAGCCAGCGTGACCATCCAAGCCAGG | |
| | GCTGAGCAGGAAGAAGAATTCATTAGTAACACTTTATTCAAGAAAATTCAGG | |
| | CTTTGCAGAAGGAGAAAGAAACCCTTGCTGTAAATTATGAGAAAGAAGAAGA | |
| | ATTCCTCACTAATGAGCTCTCCAGAAAATTGATGCAGTTGCAGCATGAGAAA | |
| | GCCGAACTAGAACAGCATCTTGAACAAGAGCAGGAATTTCAGGTCAACAAAC | |
| | TGATGAAGAAAATTAAAAAACTGGAGAATGACACCATTTCTAAGCAACTTAC | |
| | ATTAGAACAGATCCACTGTGCGACGAGCTGTGCCGCACGGTGATCGCAGCCG | |
| | CTGTCCTCTTCTCCTTCATCGTCTCGGTGCTGCTGTCTGCCTTCTGCATCCA | |
| | CTGCTACCACAAGTTTGCCCACAAGCCACCCATCTCCTCAGCTGAGATGACC | |
| | TTCCGGAGGCCCGCCCAGGCCTTCCCGGTCAGCTACTCCTCTTCCGGTGCCC | |
| | GCCGGCCCTCGCTGGACTCCATGGAGAACCAGGTCTCCGTGGATGCCTTCAA | |
| | GATCCTGGAGGATCCAAAGTGGGAATTCCCTCGGAAGAACTTGGTTCTTGGA | |
| | AAAACTCTAGGAGAAGGCGAATTTGGAAAAGTGGTCAAGGCAACGGCCTTCC | |
| | ATCTGAAAGGCAGAGCAGGGTACACCACGGTGGCCGTGAAGATGCTGAAAGA | |
| | GAACGCCTCCCCGAGTGAGCTGCGAGACCTGCTGTCAGAGTTCAACGTCCTG | |
| | AAGCAGGTCAACCACCCACATGTCATCAAATTGTATGGGGCCTGCAGCCAGG | |
| | ATGGCCCGCTCCTCCTCATCGTGGAGTACGCCAAATACGGCTCCCTGCGGGG | |
| | CTTCCTCCGCGAGAGCCGCAAAGTGGGGCCTGGCTACCTGGGCAGTGGAGGC | |
| | AGCCGCAACTCCAGCTCCCTGGACCACCCGGATGAGCGGGCCCTCACCATGG | |
| | GCGACCTCATCTCATTTGCCTGGCAGATCTCACAGGGGATGCAGTATCTGGC | |
| | CGAGATGAAGCTCGTTCATCGGGACTTGGCAGCCAGAAACATCCTGGTAGCT | |
| | GAGGGGCGGAAGATGAAGATTTCGGATTTCGGCTTGTCCCGAGATGTTTATG | |
| | AAGAGGATTCCTACGTGAAGAGGAGCCAGGGTCGGATTCCAGTTAAATGGAT | |
| | GGCAATTGAATCCCTTTTTGATCATATCTACACCACGCAAAGTGATGTATGG | |
| | TCTTTTGGTGTCCTGCTGTGGGAGATCGTGACCCTAGGGGGAAACCCCTATC | |
| | CTGGGATTCCTCCTGAGCGGCTCTTCAACCTTCTGAAGACCGGCCACCGGAT | |
| | GGAGAGGCCAGACAACTGCAGCGAGGAGATGTACCGCCTGATGCTGCAATGC | |
| | TGGAAGCAGGAGCCGGACAAAAGGCCGGTGTTTGCGGACATCAGCAAAGACC | |
| | TGGAGAAGATGATGGTTAAGAGGAGAGACTACTTGGACCTTGCGGCGTCCAC | |
| | TCCATCTGACTCCCTGATTTATGACGACGGCCTCTCAGAGGAGGAGACACCG | |
| | CTGGTGGACTGTAATAATGCCCCCCTCCCTCGAGCCCTCCCTTCCACATGGA | |
| | TTGAAAACAAACTCTATGGTAGAATTTCCCATGCATTTACTAGATTCTAG | |
| TRIM24- | ATGGAGGTGGCGGTGGAGAAGGCGGTGGCGGCGGCGGCAGCGGCCTCGGCTG | 63 |
| RET | CGGCCTCCGGGGGGCCCTCGGCGGCGCCGAGCGGGGAGAACGAGGCCGAGAG | |
| | TCGGCAGGGCCCGGACTCGGAGCGCGGCGGCGAGGCGGCCCGGCTCAACCTG | |
| | TTGGACACTTGCGCCGTGTGCCACCAGAACATCCAGAGCCGGGCGCCCAAGC | |
| | TGCTGCCCTGCCTGCACTCTTTCTGCCAGCGCTGCCTGCCCGCGCCCCAGCG | |
| | CTACCTCATGCTGCCCGCGCCCATGCTGGGCTCGGCCGAGACCCCGCCACCC | |
| | GTCCCTGCCCCCGGCTCGCCGGTCAGCGGCTCGTCGCCGTTCGCCACCCAAG | |
| | TTGGAGTCATTCGTTGCCCAGTTTGCAGCCAAGAATGTGCAGAGAGACACAT | |
| | CATAGATAACTTTTTTGTGAAGGACACTACTGAGGTTCCCAGCAGTACAGTA | |
| | GAAAAGTCAAATCAGGTATGTACAAGCTGTGAGGACAACGCAGAAGCCAATG | |
| | GGTTTTGTGTAGAGTGTGTTGAATGGCTCTGCAAGACGTGTATCAGAGCTCA | |
| | TCAGAGGGTAAAGTTCACAAAAGACCACACTGTCAGACAGAAAGAGGAAGTA | |
| | TCTCCAGAGGCAGTTGGTGTCACCAGCCAGCGACCAGTGTTTTGTCCTTTTC | |
| | ATAAAAAGGAGCAGCTGAAGCTGTACTGTGAGACATGTGACAAACTGACATG | |
| | TCGAGACTGTCAGTTGTTAGAACATAAAGAGCATAGATACCAATTTATAGAA | |
| | GAAGCTTTTCAGAATCAGAAAGTGATCATAGATACACTAATCACCAAACTGA | |
| | TGGAAAAAACAAAATACATAAAATTCACAGGAAATCAGATCCAAAACAGAAT | |
| | TATTGAAGTAAATCAAAATCAAAAGCAGGTGGAACAGGATATTAAAGTTGCT | |
| | ATATTTACACTGATGGTAGAAATAAATAAAAAAGGAAAAGCTCTACTGCATC | |
| | AGTTAGAGAGCCTTGCAAAGGACCATCGCATGAAACTTATGCAACAACAACA | |
| | GGAAGTGGCTGGACTCTCTAAACAATTGGAGCATGTCATGCATTTTTCTAAA | |
| | TGGGCAGTTTCCAGTGGCAGCAGTACAGCATTACTTTATAGCAAACGACTGA | |
| | TTACATACCGGTTACGGCACCTCCTTCGTGCAAGGTGTGATGCATCCCCAGT | |
| | GACCAACAACACCATCCAATTTCACTGTGATCCTAGTTTCTGGGCTCAAAAT | |
| | ATCATCAACTTAGGTTCTTTAGTAATCGAGGATAAAGAGAGCCAGCCACAAA | |
| | TGCCTAAGCAGAATCCTGTCGTGGAACAGAATTCACAGCCACCAAGTGGTTT | |
| | ATCATCAAACCAGTTATCCAAGTTCCCAACACAGATCAGCCTAGCTCAATTA | |
| | CGGCTCCAGCATATGCAGCAACAGCAACCGCCTCCACGTTTGATAAACTTTC | |
| | AGAATCACAGCCCCAAACCCAATGGACCAGTTCTTCCTCCTCATCCTCAACA | |
| | ACTGAGATATCCACCAAACCAGAACATACCACGACAAGCAATAAAGCCAAAC | |
| | CCCCTACAGATGGCTTTCTTGGCTCAACAAGCCATAAAACAGTGGCAGATCA | |
| | GCAGTGGACAGGGAACCCCATCAACTACCAACAGCACATCCTCTACTCCTTC | |
| | CAGCCCCACGATTACTAGTGCAGCAGGATATGATGGAAAGGCTTTTGGTTCA | |
| | CCTATGATCGATTTGAGCTCACCAGTGGGAGGGTCTTATAATCTTCCCTCTC | |
| | TTCCGGATATTGACTGTTCAAGTACTATTATGCTGGACAATATTGTGAGGAA | |
| | AGATACTAATATAGATCATGGCCAGCCAAGACCACCCTCAAACAGAACGGTC | |
| | CAGTCACCAAATTCATCAGTGCCATCTCCAGGCCTTGCAGGACCTGTTACTA | |
| | TGACTAGTGTACACCCCCCAATACGTTCACCTAGTGCCTCCAGCGTTGGAAG | |
| | CCGAGGAAGCTCTGGCTCTTCCAGCAAACCAGCAGGAGCTGACTCTACACAC | |
| | AAAGTCCCAGTGGTCATGCTGGAGCCAATTCGAATAAAACAAGAAAACAGTG | |
| | GACCACCGGAAAATTATGATTTCCCTGTTGTTATAGTGAAGCAAGAATCAGA | |
| | TGAAGAATCTAGGCCTCAAAATGCCAATTATCCAAGAAGCATACTCACCTCC | |
| | CTGCTCTTAAATAGCAGTCAGAGCTCTACTTCTGAGGAGACTGTGCTAAGAT | |
| | CAGATGCCCCTGATAGTACAGGAGATCAACCTGGACTTCACCAGGACAATTC | |
| | CTCAAATGGAAAGTCTGAATGGTTGGATCCTTCCCAGAAGTCACCTCTTCAT | |
| | GTTGGAGAGACAAGGAAAGAGGATGACCCCAATGAGGACTGGTGTGCAGTTT | |
| | GTCAAAACGGAGGGGAACTCCTCTGCTGTGAAAAGTGCCCCAAAGTATTCCA | |
| | TCTTTCTTGTCATGTGCCCACATTGACAAATTTTCCAAGTGGAGAGTGGATT | |
| | TGCACTTTCTGCCGAGACTTATCTAAACCAGAAGTTGAATATGATTGTGATG | |
| | CTCCCAGTCACAACTCAGAAAAAAAGAAAACTGAAGGCCTTGTTAAGTTAAC | |
| | ACCTATAGATAAAAGGAAGTGTGAGCGCCTACTTTTATTTCTTTACTGCCAT | |
| | GAAATGAGCCTGGCTTTTCAAGACCCTGTTCCTCTAACTGAGGATCCAAAGT | |
| | GGGAATTCCCTCGGAAGAACTTGGTTCTTGGAAAAACTCTAGGAGAAGGCGA | |
| | ATTTGGAAAAGTGGTCAAGGCAACGGCCTTCCATCTGAAAGGCAGAGCAGGG | |
| | TACACCACGGTGGCCGTGAAGATGCTGAAAGAGAACGCCTCCCCGAGTGAGC | |
| | TGCGAGACCTGCTGTCAGAGTTCAACGTCCTGAAGCAGGTCAACCACCCACA | |
| | TGTCATCAAATTGTATGGGGCCTGCAGCCAGGATGGCCCGCTCCTCCTCATC | |
| | GTGGAGTACGCCAAATACGGCTCCCTGCGGGGCTTCCTCCGCGAGAGCCGCA | |
| | AAGTGGGGCCTGGCTACCTGGGCAGTGGAGGCAGCCGCAACTCCAGCTCCCT | |
| | GGACCACCCGGATGAGCGGGCCCTCACCATGGGCGACCTCATCTCATTTGCC | |
| | TGGCAGATCTCACAGGGGATGCAGTATCTGGCCGAGATGAAGCTCGTTCATC | |
| | GGGACTTGGCAGCCAGAAACATCCTGGTAGCTGAGGGGCGGAAGATGAAGAT | |
| | TTCGGATTTCGGCTTGTCCCGAGATGTTTATGAAGAGGATTCCTACGTGAAG | |
| | AGGAGCCAGGGTCGGATTCCAGTTAAATGGATGGCAATTGAATCCCTTTTTG | |
| | ATCATATCTACACCACGCAAAGTGATGTATGGTCTTTTGGTGTCCTGCTGTG | |
| | GGAGATCGTGACCCTAGGGGGAAACCCCTATCCTGGGATTCCTCCTGAGCGG | |
| | CTCTTCAACCTTCTGAAGACCGGCCACCGGATGGAGAGGCCAGACAACTGCA | |
| | GCGAGGAGATGTACCGCCTGATGCTGCAATGCTGGAAGCAGGAGCCGGACAA | |
| | AAGGCCGGTGTTTGCGGACATCAGCAAAGACCTGGAGAAGATGATGGTTAAG | |
| | AGGAGAGACTACTTGGACCTTGCGGCGTCCACTCCATCTGACTCCCTGATTT | |
| | ATGACGACGGCCTCTCAGAGGAGGAGACACCGCTGGTGGACTGTAATAATGC | |
| | CCCCCTCCCTCGAGCCCTCCCTTCCACATGGATTGAAAACAAACTCTATGGT | |
| | AGAATTTCCCATGCATTTACTAGATTCTAG | |
| FGFR1OP- | ATGGCGGCGACGGCGGCCGCAGTGGTGGCCGAGGAGGACACGGAG | 64 |
| RET | CTGCGGGACCTGCTGGTGCAGACGCTGGAGAACAGCGGGGTCCTG | |
| | AACCGCATCAAGGCTGAACTCCGAGCAGCTGTGTTTTTAGCACTAG | |
| | AGGAGCAAGAAAAAGTAGAGAACAAAACTCCTTTAGTTAATGAGA | |
| | GCCTGAAAAAGTTTTTAAATACCAAAGACGGTCGTTTAGTGGCTAG | |
| | TCTTGTTGCAGAATTTCTTCAGTTTTTTAACCTTGACTTTACTTTGGC | |
| | TGTTTTTCAACCTGAAACTAGCACACTGCAAGGTCTCGAAGGTCGA | |
| | GAGAATTTAGCCCGAGATTTAGGTATAATTGAAGCAGAAGGTACTG | |
| | TGGGTGGACCCTTATTATTAGAAGTGATCAGGCGCTGTCAACAGAA | |
| | AGAAAAAGGGCCAACCACTGGGGAAGGTGCACTTGATCTATCTGAT | |
| | GTACATTCTCCACCAAAGTCACCAGAGGGAAAAACAAGTGCACAG | |
| | ACAACACCAAGTAAGGAGGATCCAAAGTGGGAATTCCCTCGGAAG | |
| | AACTTGGTTCTTGGAAAAACTCTAGGAGAAGGCGAATTTGGAAAAG | |
| | TGGTCAAGGCAACGGCCTTCCATCTGAAAGGCAGAGCAGGGTACA | |
| | CCACGGTGGCCGTGAAGATGCTGAAAGAGAACGCCTCCCCGAGTG | |
| | AGCTGCGAGACCTGCTGTCAGAGTTCAACGTCCTGAAGCAGGTCAA | |
| | CCACCCACATGTCATCAAATTGTATGGGGCCTGCAGCCAGGATGGC | |
| | CCGCTCCTCCTCATCGTGGAGTACGCCAAATACGGCTCCCTGCGGG | |
| | GCTTCCTCCGCGAGAGCCGCAAAGTGGGGCCTGGCTACCTGGGCAG | |
| | TGGAGGCAGCCGCAACTCCAGCTCCCTGGACCACCCGGATGAGCGG | |
| | GCCCTCACCATGGGCGACCTCATCTCATTTGCCTGGCAGATCTCACA | |
| | GGGGATGCAGTATCTGGCCGAGATGAAGCTCGTTCATCGGGACTTG | |
| | GCAGCCAGAAACATCCTGGTAGCTGAGGGGCGGAAGATGAAGATT | |
| | TCGGATTTCGGCTTGTCCCGAGATGTTTATGAAGAGGATTCCTACGT | |
| | GAAGAGGAGCCAGGGTCGGATTCCAGTTAAATGGATGGCAATTGA | |
| | ATCCCTTTTTGATCATATCTACACCACGCAAAGTGATGTATGGTCTT | |
| | TTGGTGTCCTGCTGTGGGAGATCGTGACCCTAGGGGGAAACCCCTA | |
| | TCCTGGGATTCCTCCTGAGCGGCTCTTCAACCTTCTGAAGACCGGCC | |
| | ACCGGATGGAGAGGCCAGACAACTGCAGCGAGGAGATGTACCGCC | |
| | TGATGCTGCAATGCTGGAAGCAGGAGCCGGACAAAAGGCCGGTGT | |
| | TTGCGGACATCAGCAAAGACCTGGAGAAGATGATGGTTAAGAGGA | |
| | GAGACTACTTGGACCTTGCGGCGTCCACTCCATCTGACTCCCTGATT | |
| | TATGACGACGGCCTCTCAGAGGAGGAGACACCGCTGGTGGACTGTA | |
| | ATAATGCCCCCCTCCCTCGAGCCCTCCCTTCCACATGGATTGAAAAC | |
| | AAACTCTATGGTAGAATTTCCCATGCATTTACTAGATTCTAG | |
| GAS2- | ATGTGCACTGCTCTGAGCCCAAAGGTACGCAGTGGACCTGGCCTCT | 65 |
| RET | CTGATATGCATCAGTATAGCCAATGGCTAGCCAGCAGACATGAAGC | |
| | TAATTTGCTACCAATGAAAGAAGATCTGGCCTTGTGGTTAACCAAT | |
| | CTATTAGGGAAGGAGATTACAGCAGAAACTTTTATGGAGAAGTTGG | |
| | ACAATGGTGCCTTGCTCTGTCAACTTGCAGAAACTATGCAGGAGAA | |
| | ATTCAAGGAGAGCATGGATGCTAACAAGCCCACAAAGAATCTACC | |
| | GTTGAAGAAGATCCCATGCAAAACCAGTGCACCCTCGGGCTCCTTT | |
| | TTTGCCAGAGACAATACAGCAAATTTCTTATCCTGGTGCCGAGATTT | |
| | AGGGGTGGATGAAACGTGTCTATTTGAATCGGAAGGTTTGGTCCTC | |
| | CACAAGCAACCCAGAGAAGTGTGTCTCTGTCTGCTAGAGCTTGGCC | |
| | GGATTGCAGCCAGGTATGGTGTGGAGCCTCCTGGTTTGATAAAGCT | |
| | GGAAAAAGAGATTGAACAAGAAGAAACACTTTCTGCCCCTTCTCCT | |
| | TCACCTTCTCCTTCATCAAAGTCTTCTGGAAAAAAGAGTACAGGAA | |
| | ACTTACTGGATGATGCAGTGAAACGAATTTCTGAAGATCCTCCTTG | |
| | CAAATGCCCAAACAAGTTCTGTGTGGAGCGGCTCTCCCAAGGAAGA | |
| | TACCGAGTGGGAGAAAAGATCCTCTTCATTAGGGTGAGGGTCTGCC | |
| | CTTCCGCTGCGCCCCGGACAGCCTGGAGGTGAGCACGCGCTGGGCC | |
| | CTGGACCGCGAGCAGCGGGAGAAGTACGAGCTGGTGGCCGTGTGC | |
| | ACCGTGCACGCCGGCGCGCGCGAGGAGGTGGTGATGGTGCCCTTCC | |
| | CGGTGACCGTGTACGACGAGGACGACTCGGCGCCCACCTTCCCCGC | |
| | GGGCGTCGACACCGCCAGCGCCGTGGTGGAGTTCAAGCGGAAGGA | |
| | GGACACCGTGGTGGCCACGCTGCGTGTCTTCGATGCAGACGTGGTA | |
| | CCTGCATCAGGGGAGCTGGTGAGGCGGTACACAAGCACGCTGCTCC | |
| | CCGGGGACACCTGGGCCCAGCAGACCTTCCGGGTGGAACACTGGCC | |
| | CAACGAGACCTCGGTCCAGGCCAACGGCAGCTTCGTGCGGGCGACC | |
| | GTACATGACTATAGGCTGGTTCTCAACCGGAACCTCTCCATCTCGG | |
| | AGAACCGCACCATGCAGCTGGCGGTGCTGGTCAATGACTCAGACTT | |
| | CCAGGGCCCAGGAGCGGGCGTCCTCTTGCTCCACTTCAACGTGTCG | |
| | GTGCTGCCGGTCAGCCTGCACCTGCCCAGTACCTACTCCCTCTCCGT | |
| | GAGCAGGAGGGCTCGCCGATTTGCCCAGATCGGGAAAGTCTGTGTG | |
| | GAAAACTGCCAGGCATTCAGTGGCATCAACGTCCAGTACAAGCTGC | |
| | ATTCCTCTGGTGCCAACTGCAGCACGCTAGGGGTGGTCACCTCAGC | |
| | CGAGGACACCTCGGGGATCCTGTTTGTGAATGACACCAAGGCCCTG | |
| | CGGCGGCCCAAGTGTGCCGAACTTCACTACATGGTGGTGGCCACCG | |
| | ACCAGCAGACCTCTAGGCAGGCCCAGGCCCAGCTGCTTGTAACAGT | |
| | GGAGGGGTCATATGTGGCCGAGGAGGCGGGCTGCCCCCTGTCCTGT | |
| | GCAGTCAGCAAGAGACGGCTGGAGTGTGAGGAGTGTGGCGGCCTG | |
| | GGCTCCCCAACAGGCAGGTGTGAGTGGAGGCAAGGAGATGGCAAA | |
| | GGGATCACCAGGAACTTCTCCACCTGCTCTCCCAGCACCAAGACCT | |
| | GCCCCGACGGCCACTGCGATGTTGTGGAGACCCAAGACATCAACAT | |
| | TTGCCCTCAGGACTGCCTCCGGGGCAGCATTGTTGGGGGACACGAG | |
| | CCTGGGGAGCCCCGGGGGATTAAAGCTGGCTATGGCACCTGCAACT | |
| | GCTTCCCTGAGGAGGAGAAGTGCTTCTGCGAGCCCGAAGACATCCA | |
| | GGATCCACTGTGCGACGAGCTGTGCCGCACGGTGATCGCAGCCGCT | |
| | GTCCTCTTCTCCTTCATCGTCTCGGTGCTGCTGTCTGCCTTCTGCATC | |
| | CACTGCTACCACAAGTTTGCCCACAAGCCACCCATCTCCTCAGCTG | |
| | AGATGACCTTCCGGAGGCCCGCCCAGGCCTTCCCGGTCAGCTACTC | |
| | CTCTTCCGGTGCCCGCCGGCCCTCGCTGGACTCCATGGAGAACCAG | |
| | GTCTCCGTGGATGCCTTCAAGATCCTGGAGGATCCAAAGTGGGAAT | |
| | TCCCTCGGAAGAACTTGGTTCTTGGAAAAACTCTAGGAGAAGGCGA | |
| | ATTTGGAAAAGTGGTCAAGGCAACGGCCTTCCATCTGAAAGGCAGA | |
| | GCAGGGTACACCACGGTGGCCGTGAAGATGCTGAAAGAGAACGCC | |
| | TCCCCGAGTGAGCTGCGAGACCTGCTGTCAGAGTTCAACGTCCTGA | |
| | AGCAGGTCAACCACCCACATGTCATCAAATTGTATGGGGCCTGCAG | |
| | CCAGGATGGCCCGCTCCTCCTCATCGTGGAGTACGCCAAATACGGC | |
| | TCCCTGCGGGGCTTCCTCCGCGAGAGCCGCAAAGTGGGGCCTGGCT | |
| | ACCTGGGCAGTGGAGGCAGCCGCAACTCCAGCTCCCTGGACCACCC | |
| | GGATGAGCGGGCCCTCACCATGGGCGACCTCATCTCATTTGCCTGG | |
| | CAGATCTCACAGGGGATGCAGTATCTGGCCGAGATGAAGCTCGTTC | |
| | ATCGGGACTTGGCAGCCAGAAACATCCTGGTAGCTGAGGGGCGGA | |
| | AGATGAAGATTTCGGATTTCGGCTTGTCCCGAGATGTTTATGAAGA | |
| | GGATTCCTACGTGAAGAGGAGCCAGGGTCGGATTCCAGTTAAATGG | |
| | ATGGCAATTGAATCCCTTTTTGATCATATCTACACCACGCAAAGTG | |
| | ATGTATGGTCTTTTGGTGTCCTGCTGTGGGAGATCGTGACCCTAGGG | |
| | GGAAACCCCTATCCTGGGATTCCTCCTGAGCGGCTCTTCAACCTTCT | |
| | GAAGACCGGCCACCGGATGGAGAGGCCAGACAACTGCAGCGAGGA | |
| | GATGTACCGCCTGATGCTGCAATGCTGGAAGCAGGAGCCGGACAA | |
| | AAGGCCGGTGTTTGCGGACATCAGCAAAGACCTGGAGAAGATGAT | |
| | GGTTAAGAGGAGAGACTACTTGGACCTTGCGGCGTCCACTCCATCT | |
| | GACTCCCTGATTTATGACGACGGCCTCTCAGAGGAGGAGACACCGC | |
| | TGGTGGACTGTAATAATGCCCCCCTCCCTCGAGCCCTCCCTTCCACA | |
| | TGGATTGAAAACAAACTCTATGGTAGAATTTCCCATGCATTTACTA | |
| | GATTCTAG | |
| HOOK1- | ATGGAGGAGACGCAGCCGCCGCCGCAGCCTAAGCTGCCCCTGTGCG | 66 |
| RET | ACAGCCTCATGATCTGGCTGCAGACATTCAATACTGCCTCACCTTGT | |
| | CAAGATGTCAAACAGCTGACTAGTGGAGTTGCCATGGCACAAGTTC | |
| | TTCATCAAATTGATGCAGCTTGGTTTAACGAATCTTGGTTAAGCCGA | |
| | ATTAAAGAGGATGTTGGGGACAACTGGAGAATAAAGGCCAGTAAT | |
| | GTAAAGAAGGTCCTTCAAGGAATTATGAGTTATTATCATGAGTTTTT | |
| | GGGGCAGCAGATTTCAGAAGCACTTATCCCTGATTTAAACCAAATA | |
| | ACCGAATGTTCAGATCCAGTGGAGCTTGGGAGGTTGCTCCAGCTTA | |
| | TTTTAGGTTGTGCGATCAACTGTGAAAAGAAGCAAGAACATATTCA | |
| | AAATATAATGACACTGGAAGAGTCTGTTCAACATGTGGTCATGACT | |
| | GCTATTCAAGAGTTGATGAGTAAAGAAATATTGAGCTCTCCTCCAA | |
| | ATGATGCTGTTGGAGAATTGGAGCAACAGCTTAAAAGAGCCTTGGA | |
| | AGAACTTCAGGAAGCACTAGCAGAAAAAGAAGAGCTGAGGCAAAG | |
| | ATGTGAAGAATTGGATATGCAGGTGACTACACTTCAAGATGAAAAG | |
| | AATTCACTGGTTTCTGAAAATGAGATGATGAATGAAAAACTTGACC | |
| | AGTTGGATGGCTCTTTTGATGATCCAAACACAGTGGTTGCAAAAAA | |
| | GTATTTTCATGCACAATTACAACTAGAACAATTACAGGAAGAAAAC | |
| | TTCAGGCTTGAAGCTGCAAAAGATGATTACCGTGTTCACTGTGAAG | |
| | AACTTGAAAAGCAGCTAATCGAATTCCAGCATAGGAATGATGAATT | |
| | GACTAGTCTTGCAGAAGAAACAAGAGCCCTGAAAGATGAAATAGA | |
| | TGTTCTTAGGGCTACCTCTGATAAAGCAAATAAACTGGAGTCAACA | |
| | GTTGAGATATATCGTCAGAAGCTACAAGATCTGAATGACCTTCGCA | |
| | AGCAGGTGAAAACTTTACAGGAAACCAACATGATGTATATGCATAA | |
| | TACAGTCAGCTTAGAAGAAGAATTAAAAAAAGCAAATGCAGCACG | |
| | TACACAATTAGAAACATACAAAAGGCAGGTTCAAGATCTTCATGTT | |
| | AAACTTTCCTCCGAATCCAAGAGGGCAGACACACTAGCGTTTGAAA | |
| | TGAAGCGGCTTGAAGAAAAACATGAAGCTTTACTTAAGGAAAAAG | |
| | AGAGACTAATTGAGCAGCGTGATACTTTGAAAGAAACAAATGAAG | |
| | AGCTTCGATGTTCACAAGTACAACAGGACCACCTAAACCAAACAGA | |
| | TGCATCTGCTACAAAAAGTTATGAGAATCTTGCTGCTGAGATTATG | |
| | CCAGTGGAATATAGGGAGGTGTTTATTCGACTGCAACATGAAAATA | |
| | AGATGCTTCGCTTACAGCAAGAAGGCTCTGAGAATGAACGTATTGA | |
| | GGAACTTCAGGAGCAGCTAGAACAGAAACACCGTAAAATGAATGA | |
| | ACTGGAAACTGAGCAGAGGCTGAGCAAAGAGCGTATTAGAGAATT | |
| | GCAGCAGCAGATTGAGGACCTCCAGAAATCTTTACAGGAACAAGG | |
| | TTCCAAGTCTGAAGGCGAAAGTTCCAGCAAATTAAAGCAGAAGTTG | |
| | GAAGCTCATATGGAAAAACTCACAGAGGTCCATGAAGAATTACAG | |
| | AAGAAACAAGAACTCATTGAAGATCTTCAGCCAGATATAAATCAA | |
| | AATGTACAAAAGATCAATGAACTTGAAGCTGCTCTTCAGAAGAAAG | |
| | ATGAAGATATGAAAGCAATGGAGGAAAGATATAAAATGTACTTGG | |
| | AGAAAGCCAGAAATGTAATAAAAACTTTGGATCCCAAGTTAAATCC | |
| | AGCATCAGCTGAAATAATGCTACTAAGAAAGCAGTTGGCAGAGAA | |
| | AGAGAGAAGAATTGAGATTCTGGAGGAGGATCCAAAGTGGGAATT | |
| | CCCTCGGAAGAACTTGGTTCTTGGAAAAACTCTAGGAGAAGGCGAA | |
| | TTTGGAAAAGTGGTCAAGGCAACGGCCTTCCATCTGAAAGGCAGAG | |
| | CAGGGTACACCACGGTGGCCGTGAAGATGCTGAAAGAGAACGCCT | |
| | CCCCGAGTGAGCTGCGAGACCTGCTGTCAGAGTTCAACGTCCTGAA | |
| | GCAGGTCAACCACCCACATGTCATCAAATTGTATGGGGCCTGCAGC | |
| | CAGGATGGCCCGCTCCTCCTCATCGTGGAGTACGCCAAATACGGCT | |
| | CCCTGCGGGGCTTCCTCCGCGAGAGCCGCAAAGTGGGGCCTGGCTA | |
| | CCTGGGCAGTGGAGGCAGCCGCAACTCCAGCTCCCTGGACCACCCG | |
| | GATGAGCGGGCCCTCACCATGGGCGACCTCATCTCATTTGCCTGGC | |
| | AGATCTCACAGGGGATGCAGTATCTGGCCGAGATGAAGCTCGTTCA | |
| | TCGGGACTTGGCAGCCAGAAACATCCTGGTAGCTGAGGGGCGGAA | |
| | GATGAAGATTTCGGATTTCGGCTTGTCCCGAGATGTTTATGAAGAG | |
| | GATTCCTACGTGAAGAGGAGCCAGGGTCGGATTCCAGTTAAATGGA | |
| | TGGCAATTGAATCCCTTTTTGATCATATCTACACCACGCAAAGTGAT | |
| | GTATGGTCTTTTGGTGTCCTGCTGTGGGAGATCGTGACCCTAGGGG | |
| | GAAACCCCTATCCTGGGATTCCTCCTGAGCGGCTCTTCAACCTTCTG | |
| | AAGACCGGCCACCGGATGGAGAGGCCAGACAACTGCAGCGAGGAG | |
| | ATGTACCGCCTGATGCTGCAATGCTGGAAGCAGGAGCCGGACAAA | |
| | AGGCCGGTGTTTGCGGACATCAGCAAAGACCTGGAGAAGATGATG | |
| | GTTAAGAGGAGAGACTACTTGGACCTTGCGGCGTCCACTCCATCTG | |
| | ACTCCCTGATTTATGACGACGGCCTCTCAGAGGAGGAGACACCGCT | |
| | GGTGGACTGTAATAATGCCCCCCTCCCTCGAGCCCTCCCTTCCACAT | |
| | GGATTGAAAACAAACTCTATGGTAGAATTTCCCATGCATTTACTAG | |
| | ATTCTAG | |
| LMNA- | ATGGAGACCCCGTCCCAGCGGCGCGCCACCCGCAGCGGGGCGCAG | 147 |
| RET | GCCAGCTCCACTCCGCTGTCGCCCACCCGCATCACCCGGCTGCAGG | |
| | AGAAGGAGGACCTGCAGGAGCTCAATGATCGCTTGGCGGTCTACAT | |
| | CGACCGTGTGCGCTCGCTGGAAACGGAGAACGCAGGGCTGCGCCTT | |
| | CGCATCACCGAGTCTGAAGAGGTGGTCAGCCGCGAGGTGTCCGGCA | |
| | TCAAGGCCGCCTACGAGGCCGAGCTCGGGGATGCCCGCAAGACCCT | |
| | TGACTCAGTAGCCAAGGAGCGCGCCCGCCTGCAGCTGGAGCTGAGC | |
| | AAAGTGCGTGAGGAGTTTAAGGAGCTGAAAGCGCGCAATACCAAG | |
| | AAGGAGGGTGACCTGATAGCTGCTCAGGCTCGGCTGAAGGACCTG | |
| | GAGGCTCTGCTGAACTCCAAGGAGGCCGCACTGAGCACTGCTCTCA | |
| | GTGAGAAGCGCACGCTGGAGGGCGAGCTGCATGATCTGCGGGGCC | |
| | AGGTGGCCAAGGAGGATCCAAAGTGGGAATTCCCTCGGAAGAACT | |
| | TGGTTCTTGGAAAAACTCTAGGAGAAGGCGAATTTGGAAAAGTGGT | |
| | CAAGGCAACGGCCTTCCATCTGAAAGGCAGAGCAGGGTACACCAC | |
| | GGTGGCCGTGAAGATGCTGAAAGAGAACGCCTCCCCGAGTGAGCT | |
| | GCGAGACCTGCTGTCAGAGTTCAACGTCCTGAAGCAGGTCAACCAC | |
| | CCACATGTCATCAAATTGTATGGGGCCTGCAGCCAGGATGGCCCGC | |
| | TCCTCCTCATCGTGGAGTACGCCAAATACGGCTCCCTGCGGGGCTT | |
| | CCTCCGCGAGAGCCGCAAAGTGGGGCCTGGCTACCTGGGCAGTGG | |
| | AGGCAGCCGCAACTCCAGCTCCCTGGACCACCCGGATGAGCGGGCC | |
| | CTCACCATGGGCGACCTCATCTCATTTGCCTGGCAGATCTCACAGG | |
| | GGATGCAGTATCTGGCCGAGATGAAGCTCGTTCATCGGGACTTGGC | |
| | AGCCAGAAACATCCTGGTAGCTGAGGGGCGGAAGATGAAGATTTC | |
| | GGATTTCGGCTTGTCCCGAGATGTTTATGAAGAGGATTCCTACGTG | |
| | AAGAGGAGCCAGGGTCGGATTCCAGTTAAATGGATGGCAATTGAA | |
| | TCCCTTTTTGATCATATCTACACCACGCAAAGTGATGTATGGTCTTT | |
| | TGGTGTCCTGCTGTGGGAGATCGTGACCCTAGGGGGAAACCCCTAT | |
| | CCTGGGATTCCTCCTGAGCGGCTCTTCAACCTTCTGAAGACCGGCC | |
| | ACCGGATGGAGAGGCCAGACAACTGCAGCGAGGAGATGTACCGCC | |
| | TGATGCTGCAATGCTGGAAGCAGGAGCCGGACAAAAGGCCGGTGT | |
| | TTGCGGACATCAGCAAAGACCTGGAGAAGATGATGGTTAAGAGGA | |
| | GAGACTACTTGGACCTTGCGGCGTCCACTCCATCTGACTCCCTGATT | |
| | TATGACGACGGCCTCTCAGAGGAGGAGACACCGCTGGTGGACTGTA | |
| | ATAATGCCCCCCTCCCTCGAGCCCTCCCTTCCACATGGATTGAAAAC | |
| | AAACTCTATGGTAGAATTTCCCATGCATTTACTAGATTCTAG | |
| LMNA- | ATGGAGACCCCGTCCCAGCGGCGCGCCACCCGCAGCGGGGCGCAG | 148 |
| RET | GCCAGCTCCACTCCGCTGTCGCCCACCCGCATCACCCGGCTGCAGG | |
| | AGAAGGAGGACCTGCAGGAGCTCAATGATCGCTTGGCGGTCTACAT | |
| | CGACCGTGTGCGCTCGCTGGAAACGGAGAACGCAGGGCTGCGCCTT | |
| | CGCATCACCGAGTCTGAAGAGGTGGTCAGCCGCGAGGTGTCCGGCA | |
| | TCAAGGCCGCCTACGAGGCCGAGCTCGGGGATGCCCGCAAGACCCT | |
| | TGACTCAGTAGCCAAGGAGCGCGCCCGCCTGCAGCTGGAGCTGAGC | |
| | AAAGTGCGTGAGGAGTTTAAGGAGCTGAAAGCGCGCAATACCAAG | |
| | AAGGAGGGTGACCTGATAGCTGCTCAGGCTCGGCTGAAGGACCTG | |
| | GAGGCTCTGCTGAACTCCAAGGAGGCCGCACTGAGCACTGCTCTCA | |
| | GTGAGAAGCGCACGCTGGAGGGCGAGCTGCATGATCTGCGGGGCC | |
| | AGGTGGCCAAGGAGGATCCAAAGTGGGAATTCCCTCGGAAGAACT | |
| | TGGTTCTTGGAAAAACTCTAGGAGAAGGCGAATTTGGAAAAGTGGT | |
| | CAAGGCAACGGCCTTCCATCTGAAAGGCAGAGCAGGGTACACCAC | |
| | GGTGGCCGTGAAGATGCTGAAAGAGAACGCCTCCCCGAGTGAGCT | |
| | GCGAGACCTGCTGTCAGAGTTCAACGTCCTGAAGCAGGTCAACCAC | |
| | CCACATGTCATCAAATTGTATGGGGCCTGCAGCCAGGATGGCCCGC | |
| | TCCTCCTCATCGTGGAGTACGCCAAATACGGCTCCCTGCGGGGCTT | |
| | CCTCCGCGAGAGCCGCAAAGTGGGGCCTGGCTACCTGGGCAGTGG | |
| | AGGCAGCCGCAACTCCAGCTCCCTGGACCACCCGGATGAGCGGGCC | |
| | CTCACCATGGGCGACCTCATCTCATTTGCCTGGCAGATCTCACAGG | |
| | GGATGCAGTATCTGGCCGAGATGAAGCTCGTTCATCGGGACTTGGC | |
| | AGCCAGAAACATCCTGGTAGCTGAGGGGCGGAAGATGAAGATTTC | |
| | GGATTTCGGCTTGTCCCGAGATGTTTATGAAGAGGATTCCTACGTG | |
| | AAGAGGAGCCAGGGTCGGATTCCAGTTAAATGGATGGCAATTGAA | |
| | TCCCTTTTTGATCATATCTACACCACGCAAAGTGATGTATGGTCTTT | |
| | TGGTGTCCTGCTGTGGGAGATCGTGACCCTAGGGGGAAACCCCTAT | |
| | CCTGGGATTCCTCCTGAGCGGCTCTTCAACCTTCTGAAGACCGGCC | |
| | ACCGGATGGAGAGGCCAGACAACTGCAGCGAGGAGATGTACCGCC | |
| | TGATGCTGCAATGCTGGAAGCAGGAGCCGGACAAAAGGCCGGTGT | |
| | TTGCGGACATCAGCAAAGACCTGGAGAAGATGATGGTTAAGAGGA | |
| | GAGACTACTTGGACCTTGCGGCGTCCACTCCATCTGACTCCCTGATT | |
| MLPH- | TATGACGACGGCCTCTCAGAGGAGGAGACACCGCTGGTGGACTGTA | 149 |
| RET | ATAATGCCCCCCTCCCTCGAGCCCTCCCTTCCACATGGATTGAAAAC | |
| | AAACTCTATGGTAGAATTTCCCATGCATTTACTAGATTCTAG | |
| | ATGGGGAAGAAACTGGATCTTTCCAAGCTCACTGATGAAGAGGCCC | |
| | AGCATGTCTTGGAAGTTGTTCAACGAGATTTTGACCTCCGAAGGAA | |
| | AGAAGAGGAACGGCTAGAGGCGTTGAAGGGCAAGATTAAGAAGGA | |
| | AAGCTCCAAGAGGGAGCTGCTTTCCGACACTGCCCATCTGAACGAG | |
| | ACCCACTGCGCCCGCTGCCTGCAGCCCTACCAGCTGCTTGTGAATA | |
| | GCAAAAGGCAGTGCCTGGAATGTGGCCTCTTCACCTGCAAAAGCTG | |
| | TGGCCGCGTCCACCCGGAGGAGCAGGGCTGGATCTGTGACCCCTGC | |
| | CATCTGGCCAGAGTCGTGAAGATCGGCTCACTGGAGTGGTACTATG | |
| | AGCATGTGAAAGCCCGCTTCAAGAGGTTCGGAAGTGCCAAGGTCAT | |
| | CCGGTCCCTCCACGGGCGGCTGCAGGGTGGAGCTGGGCCTGAACTG | |
| | ATATCTGAAGAGAGAAGTGGAGACAGCGACCAGACAGATGAGGAT | |
| | GGAGAACCTGGCTCAGAGGCCCAGGCCCAGGCCCAGCCCTTTGGCA | |
| | GCAAAAAAAAGCGCCTCCTCTCCGTCCACGACTTCGACTTCGAGGG | |
| | AGACTCAGATGACTCCACTCAGCCTCAAGGTCACTCCCTGCACCTG | |
| | TCCTCAGTCCCTGAGGCCAGGGACAGCCCACAGTCCCTCACAGATG | |
| | AGTCCTGCTCAGAGAAGGCAGCCCCTCACAAGGCTGAGGGCCTGG | |
| | AGGAGGCTGATACTGGGGCCTCTGGGTGCCACTCCCATCCGGAAGA | |
| | GCAGCCGACCAGCATCTCACCTTCCAGACACGGCGCCCTGGCTGAG | |
| | CTCTGCCCGCCTGGAGGCTCCCACAGGATGGCCCTGGGGACTGCTG | |
| | CTGCACTCGGGTCGAATGTCATCAGGAATGAGCAGCTGCCCCTGCA | |
| | GTACTTGGCCGATGTGGACACCTCTGATGAGGAAAGCATCCGGGCT | |
| | CACGTGATGGCCTCCCACCATTCCAAGCGGAGAGGCCGGGCGTCTT | |
| | CTGAGAGTCAGATCTTTGAGCTGAATAAGCATATTTCAGCTGTGGA | |
| | ATGCCTGCTGACCTACCTGGAGAACACAGTTGTGCCTCCCTTGGCC | |
| | AAGGAGGATCCAAAGTGGGAATTCCCTCGGAAGAACTTGGTTCTTG | |
| | GAAAAACTCTAGGAGAAGGCGAATTTGGAAAAGTGGTCAAGGCAA | |
| | CGGCCTTCCATCTGAAAGGCAGAGCAGGGTACACCACGGTGGCCGT | |
| | GAAGATGCTGAAAGAGAACGCCTCCCCGAGTGAGCTGCGAGACCT | |
| | GCTGTCAGAGTTCAACGTCCTGAAGCAGGTCAACCACCCACATGTC | |
| | ATCAAATTGTATGGGGCCTGCAGCCAGGATGGCCCGCTCCTCCTCA | |
| | TCGTGGAGTACGCCAAATACGGCTCCCTGCGGGGCTTCCTCCGCGA | |
| | GAGCCGCAAAGTGGGGCCTGGCTACCTGGGCAGTGGAGGCAGCCG | |
| | CAACTCCAGCTCCCTGGACCACCCGGATGAGCGGGCCCTCACCATG | |
| | GGCGACCTCATCTCATTTGCCTGGCAGATCTCACAGGGGATGCAGT | |
| | ATCTGGCCGAGATGAAGCTCGTTCATCGGGACTTGGCAGCCAGAAA | |
| | CATCCTGGTAGCTGAGGGGCGGAAGATGAAGATTTCGGATTTCGGC | |
| | TTGTCCCGAGATGTTTATGAAGAGGATTCCTACGTGAAGAGGAGCC | |
| | AGGGTCGGATTCCAGTTAAATGGATGGCAATTGAATCCCTTTTTGA | |
| | TCATATCTACACCACGCAAAGTGATGTATGGTCTTTTGGTGTCCTGC | |
| | TGTGGGAGATCGTGACCCTAGGGGGAAACCCCTATCCTGGGATTCC | |
| | TCCTGAGCGGCTCTTCAACCTTCTGAAGACCGGCCACCGGATGGAG | |
| | AGGCCAGACAACTGCAGCGAGGAGATGTACCGCCTGATGCTGCAA | |
| | TGCTGGAAGCAGGAGCCGGACAAAAGGCCGGTGTTTGCGGACATC | |
| | AGCAAAGACCTGGAGAAGATGATGGTTAAGAGGAGAGACTACTTG | |
| | GACCTTGCGGCGTCCACTCCATCTGACTCCCTGATTTATGACGACGG | |
| | CCTCTCAGAGGAGGAGACACCGCTGGTGGACTGTAATAATGCCCCC | |
| | CTCCCTCGAGCCCTCCCTTCCACATGGATTGAAAACAAACTCTATG | |
| | GTAGAATTTCCCATGCATTTACTAGATTCTAG | |
| MYH9- | ATGGCACAGCAAGCTGCCGATAAGTATCTCTATGTGGATAAAAACT | 150 |
| RET | TCATCAACAATCCGCTGGCCCAGGCCGACTGGGCTGCCAAGAAGCT | |
| | GGTATGGGTGCCTTCCGACAAGAGTGGCTTTGAGCCAGCCAGCCTC | |
| | AAGGAGGAGGTGGGCGAAGAGGCCATCGTGGAGCTGGTGGAGAAT | |
| | GGGAAGAAGGTGAAGGTGAACAAGGATGACATCCAGAAGATGAAC | |
| | CCGCCCAAGTTCTCCAAGGTGGAGGACATGGCAGAGCTCACGTGCC | |
| | TCAACGAAGCCTCGGTGCTGCACAACCTCAAGGAGCGTTACTACTC | |
| | AGGGCTCATCTACACCTATTCAGGCCTGTTCTGTGTGGTCATCAATC | |
| | CTTACAAGAACCTGCCCATCTACTCTGAAGAGATTGTGGAAATGTA | |
| | CAAGGGCAAGAAGAGGCACGAGATGCCCCCTCACATCTATGCCATC | |
| | ACAGACACCGCCTACAGGAGTATGATGCAAGACCGAGAAGATCAA | |
| | TCCATCTTGTGCACTGGTGAATCTGGAGCTGGCAAGACGGAGAACA | |
| | CCAAGAAGGTCATCCAGTATCTGGCGTACGTGGCGTCCTCGCACAA | |
| | GAGCAAGAAGGACCAGGGCGAGCTGGAGCGGCAGCTGCTGCAGGC | |
| | CAACCCCATCCTGGAGGCCTTCGGGAACGCCAAGACCGTGAAGAAT | |
| | GACAACTCCTCCCGCTTCGGCAAATTCATTCGCATCAACTTTGATGT | |
| | CAATGGCTACATTGTTGGAGCCAACATTGAGACTTATCTTTTGGAG | |
| | AAATCTCGTGCTATCCGCCAAGCCAAGGAAGAACGGACCTTCCACA | |
| | TCTTCTATTATCTCCTGTCTGGGGCTGGAGAGCACCTGAAGACCGAT | |
| | CTCCTGTTGGAGCCGTACAACAAATACCGCTTCCTGTCCAATGGAC | |
| | ACGTCACCATCCCCGGGCAGCAGGACAAGGACATGTTCCAGGAGA | |
| | CCATGGAGGCCATGAGGATTATGGGCATCCCAGAAGAGGAGCAAA | |
| | TGGGCCTGCTGCGGGTCATCTCAGGGGTTCTTCAGCTCGGCAACAT | |
| | CGTCTTCAAGAAGGAGCGGAACACTGACCAGGCGTCCATGCCCGAC | |
| | AACACAGCTGCCCAAAAGGTGTCCCATCTCTTGGGTATCAATGTGA | |
| | CCGATTTCACCAGAGGAATCCTCACCCCGCGCATCAAGGTGGGACG | |
| | GGATTACGTCCAGAAGGCGCAGACTAAAGAGCAGGCTGACTTTGCC | |
| | ATCGAGGCCTTGGCCAAGGCGACCTATGAGCGGATGTTCCGCTGGC | |
| | TGGTGCTGCGCATCAACAAGGCTCTGGACAAGACCAAGAGGCAGG | |
| | GCGCCTCCTTCATCGGGATCCTGGACATTGCCGGCTTCGAGATCTTT | |
| | GATCTGAACTCGTTTGAGCAGCTGTGCATCAATTACACCAATGAGA | |
| | AGCTGCAGCAGCTCTTCAACCACACCATGTTCATCCTGGAGCAGGA | |
| | GGAGTACCAGCGCGAGGGCATCGAGTGGAACTTCATCGACTTTGGC | |
| | CTCGACCTGCAGCCCTGCATCGACCTCATTGAGAAGCCAGCAGGCC | |
| | CCCCGGGCATTCTGGCCCTGCTGGACGAGGAGTGCTGGTTCCCCAA | |
| | AGCCACCGACAAGAGCTTCGTGGAGAAGGTGATGCAGGAGCAGGG | |
| | CACCCACCCCAAGTTCCAGAAGCCCAAGCAGCTGAAGGACAAAGC | |
| | TGATTTCTGCATTATCCACTATGCCGGCAAGGTGGATTACAAAGCT | |
| | GACGAGTGGCTGATGAAGAACATGGATCCCCTGAATGACAACATC | |
| | GCCACACTGCTCCACCAGTCCTCTGACAAGTTTGTCTCGGAGCTGTG | |
| | GAAGGATGTGGACCGCATCATCGGCCTGGACCAGGTGGCCGGCAT | |
| | GTCGGAGACCGCACTGCCCGGGGCCTTCAAGACGCGGAAGGGCAT | |
| | GTTCCGCACTGTGGGGCAGCTTTACAAGGAGCAGCTGGCCAAGCTG | |
| | ATGGCTACGCTGAGGAACACGAACCCCAACTTTGTCCGCTGCATCA | |
| | TCCCCAACCACGAGAAGAAGGCCGGCAAGCTGGACCCGCATCTCGT | |
| | GCTGGACCAGCTGCGCTGCAACGGTGTTCTCGAGGGCATCCGTATC | |
| | TGCCGCCAGGGCTTCCCCAACAGGGTGGTCTTCCAGGAGTTTCGGC | |
| | AGAGATATGAGATCCTGACTCCAAACTCCATTCCCAAGGGTTTCAT | |
| | GGACGGGAAGCAGGCGTGCGTGCTCATGATAAAAGCCCTGGAGCT | |
| | CGACAGCAATCTGTACCGCATTGGCCAGAGCAAAGTCTTCTTCCGT | |
| | GCCGGTGTGCTGGCCCACCTGGAGGAGGAGCGAGACCTGAAGATC | |
| | ACCGACGTCATCATAGGGTTCCAGGCCTGCTGCAGGGGCTACCTGG | |
| | CCAGGAAAGCATTTGCCAAGCGGCAGCAGCAGCTTACCGCCATGA | |
| | AGGTCCTCCAGCGGAACTGCGCTGCCTACCTGAAGCTGCGGAACTG | |
| | GCAGTGGTGGCGGCTCTTCACCAAGGTCAAGCCGCTGCTGCAGGTG | |
| | AGCCGGCAGGAGGAGGAGATGATGGCCAAGGAGGAGGAGCTGGTG | |
| | AAGGTCAGAGAGAAGCAGCTGGCTGCGGAGAACAGGCTCACGGAG | |
| | ATGGAGACGCTGCAGTCTCAGCTCATGGCAGAGAAATTGCAGCTGC | |
| | AGGAGCAGCTCCAGGCAGAAACCGAGCTGTGTGCCGAGGCTGAGG | |
| | AGCTCCGGGCCCGCCTGACCGCCAAGAAGCAGGAATTAGAAGAGA | |
| | TCTGCCATGACCTAGAGGCCAGGGTGGAGGAGGAGGAGGAGCGCT | |
| | GCCAGCACCTGCAGGCGGAGAAGAAGAAGATGCAGCAGAACATCC | |
| | AGGAGCTTGAGGAGCAGCTGGAGGAGGAGGAGAGCGCCCGGCAGA | |
| | AGCTGCAGCTGGAGAAGGTGACCACCGAGGCGAAGCTGAAAAAGC | |
| | TGGAGGAGGAGCAGATCATCCTGGAGGACCAGAACTGCAAGCTGG | |
| | CCAAGGAAAAGAAACTGCTGGAAGACAGAATAGCTGAGTTCACCA | |
| | CCAACCTCACAGAAGAGGAGGAGAAATCTAAGAGCCTCGCCAAGC | |
| | TCAAGAACAAGCATGAGGCAATGATCACTGACTTGGAAGAGCGCC | |
| | TCCGCAGGGAGGAGAAGCAGCGACAGGAGCTGGAGAAGACCCGCC | |
| | GGAAGCTGGAGGGAGACTCCACAGACCTCAGCGACCAGATCGCCG | |
| | AGCTCCAGGCCCAGATCGCGGAGCTCAAGATGCAGCTGGCCAAGA | |
| | AAGAGGAGGAGCTCCAGGCCGCCCTGGCCAGAGTGGAAGAGGAAG | |
| | CTGCCCAGAAGAACATGGCCCTCAAGAAGATCCGGGAGCTGGAAT | |
| | CTCAGATCTCTGAACTCCAGGAAGACCTGGAGTCTGAGCGTGCTTC | |
| | CAGGAATAAAGCTGAGAAGCAGAAACGGGACCTTGGGGAAGAGCT | |
| | AGAGGCTCTGAAAACAGAGTTGGAGGACACGCTGGATTCCACAGC | |
| | TGCCCAGCAGGAGCTCAGGTCAAAACGTGAGCAGGAGGTGAACAT | |
| | CCTGAAGAAGACCCTGGAGGAGGAGGCCAAGACCCACGAGGCCCA | |
| | GATCCAGGAGATGAGGCAGAAGCACTCACAGGCCGTGGAGGAGCT | |
| | GGCGGAGCAGCTGGAGCAGACGAAGCGGGTGAAAGCAAACCTCGA | |
| | GAAGGCAAAGCAGACTCTGGAGAACGAGCGGGGGGAGCTGGCCAA | |
| | CGAGGTGAAGGTGCTGCTGCAGGGCAAAGGGGACTCGGAGCACAA | |
| | GCGCAAGAAAGTGGAGGCGCAGCTGCAGGAGCTGCAGGTCAAGTT | |
| | CAACGAGGGAGAGCGCGTGCGCACAGAGCTGGCCGACAAGGTCAC | |
| | CAAGCTGCAGGTGGAGCTGGACAACGTGACCGGGCTTCTCAGCCAG | |
| | TCCGACAGCAAGTCCAGCAAGCTCACCAAGGACTTCTCCGCGCTGG | |
| | AGTCCCAGCTGCAGGACACTCAGGAGCTGCTGCAGGAGGAGAACC | |
| | GGCAGAAGCTGAGCCTGAGCACCAAGCTCAAGCAGGTGGAGGACG | |
| | AGAAGAATTCCTTCCGGGAGCAGCTGGAGGAGGAGGAGGAGGCCA | |
| | AGCACAACCTGGAGAAGCAGATCGCCACCCTCCATGCCCAGGTGGC | |
| | CGACATGAAAAAGAAGATGGAGGACAGTGTGGGGTGCCTGGAAAC | |
| | TGCTGAGGAGGTGAAGAGGAAGCTCCAGAAGGACCTGGAGGGCCT | |
| | GAGCCAGCGGCACGAGGAGAAGGTGGCCGCCTACGACAAGCTGGA | |
| | GAAGACCAAGACGCGGCTGCAGCAGGAGCTGGACGACCTGCTGGT | |
| | GGACCTGGACCACCAGCGCCAGAGCGCGTGCAACCTGGAGAAGAA | |
| | GCAGAAGAAGTTTGACCAGCTCCTGGCGGAGGAGAAGACCATCTCT | |
| | GCCAAGTATGCAGAGGAGCGCGACCGGGCTGAGGCGGAGGCCCGA | |
| | GAGAAGGAGACCAAGGCTCTGTCGCTGGCCCGGGCCCTGGAGGAA | |
| | GCCATGGAGCAGAAGGCGGAGCTGGAGCGGCTCAACAAGCAGTTC | |
| | CGCACGGAGATGGAGGACCTTATGAGCTCCAAGGATGATGTGGGC | |
| | AAGAGTGTCCACGAGCTGGAGAAGTCCAAGCGGGCCCTAGAGCAG | |
| | CAGGTGGAGGAGATGAAGACGCAGCTGGAAGAGCTGGAGGACGAG | |
| | CTGCAGGCCACCGAAGATGCCAAGCTGCGGTTGGAGGTCAACCTGC | |
| | AGGCCATGAAGGCCCAGTTCGAGCGGGACCTGCAGGGCCGGGACG | |
| | AGCAGAGCGAGGAGAAGAAGAAGCAGCTGGTCAGACAGGTGCGG | |
| | GAGATGGAGGCAGAGCTGGAGGACGAGAGGAAGCAGCGCTCGATG | |
| | GCAGTGGCCGCCCGGAAGAAGCTGGAGATGGACCTGAAGGACCTG | |
| | GAGGCGCACATCGACTCGGCCAACAAGAACCGGGACGAAGCCATC | |
| | AAACAGCTGCGGAAGCTGCAGGAGGATCCAAAGTGGGAATTCCCT | |
| | CGGAAGAACTTGGTTCTTGGAAAAACTCTAGGAGAAGGCGAATTTG | |
| | GAAAAGTGGTCAAGGCAACGGCCTTCCATCTGAAAGGCAGAGCAG | |
| | GGTACACCACGGTGGCCGTGAAGATGCTGAAAGAGAACGCCTCCC | |
| | CGAGTGAGCTGCGAGACCTGCTGTCAGAGTTCAACGTCCTGAAGCA | |
| | GGTCAACCACCCACATGTCATCAAATTGTATGGGGCCTGCAGCCAG | |
| | GATGGCCCGCTCCTCCTCATCGTGGAGTACGCCAAATACGGCTCCC | |
| | TGCGGGGCTTCCTCCGCGAGAGCCGCAAAGTGGGGCCTGGCTACCT | |
| | GGGCAGTGGAGGCAGCCGCAACTCCAGCTCCCTGGACCACCCGGAT | |
| | GAGCGGGCCCTCACCATGGGCGACCTCATCTCATTTGCCTGGCAGA | |
| | TCTCACAGGGGATGCAGTATCTGGCCGAGATGAAGCTCGTTCATCG | |
| | GGACTTGGCAGCCAGAAACATCCTGGTAGCTGAGGGGCGGAAGAT | |
| | GAAGATTTCGGATTTCGGCTTGTCCCGAGATGTTTATGAAGAGGAT | |
| | TCCTACGTGAAGAGGAGCCAGGGTCGGATTCCAGTTAAATGGATGG | |
| | CAATTGAATCCCTTTTTGATCATATCTACACCACGCAAAGTGATGTA | |
| | TGGTCTTTTGGTGTCCTGCTGTGGGAGATCGTGACCCTAGGGGGAA | |
| | ACCCCTATCCTGGGATTCCTCCTGAGCGGCTCTTCAACCTTCTGAAG | |
| | ACCGGCCACCGGATGGAGAGGCCAGACAACTGCAGCGAGGAGATG | |
| | TACCGCCTGATGCTGCAATGCTGGAAGCAGGAGCCGGACAAAAGG | |
| | CCGGTGTTTGCGGACATCAGCAAAGACCTGGAGAAGATGATGGTTA | |
| | AGAGGAGAGACTACTTGGACCTTGCGGCGTCCACTCCATCTGACTC | |
| | CCTGATTTATGACGACGGCCTCTCAGAGGAGGAGACACCGCTGGTG | |
| | GACTGTAATAATGCCCCCCTCCCTCGAGCCCTCCCTTCCACATGGAT | |
| | TGAAAACAAACTCTATGGTAGAATTTCCCATGCATTTACTAGATTCT | |
| | AG | |
| PCDH15- | ATGTTTCGACAGTTTTATCTCTGGACATGTTTAGCTTCAGGGATCAT | 151 |
| RET | CCTGGGCTCTCTCTTTGAAATCTGCTTGGGCCAGTATGATGATGGAC | |
| | ACCGTGGTGGCCACGCTGCGTGTCTTCGATGCAGACGTGGTACCTG | |
| | CATCAGGGGAGCTGGTGAGGCGGTACACAAGCACGCTGCTCCCCG | |
| | GGGACACCTGGGCCCAGCAGACCTTCCGGGTGGAACACTGGCCCA | |
| | ACGAGACCTCGGTCCAGGCCAACGGCAGCTTCGTGCGGGCGACCGT | |
| | ACATGACTATAGGCTGGTTCTCAACCGGAACCTCTCCATCTCGGAG | |
| | AACCGCACCATGCAGCTGGCGGTGCTGGTCAATGACTCAGACTTCC | |
| | AGGGCCCAGGAGCGGGCGTCCTCTTGCTCCACTTCAACGTGTCGGT | |
| | GCTGCCGGTCAGCCTGCACCTGCCCAGTACCTACTCCCTCTCCGTGA | |
| | GCAGGAGGGCTCGCCGATTTGCCCAGATCGGGAAAGTCTGTGTGGA | |
| | AAACTGCCAGGCATTCAGTGGCATCAACGTCCAGTACAAGCTGCAT | |
| | TCCTCTGGTGCCAACTGCAGCACGCTAGGGGTGGTCACCTCAGCCG | |
| | AGGACACCTCGGGGATCCTGTTTGTGAATGACACCAAGGCCCTGCG | |
| | GCGGCCCAAGTGTGCCGAACTTCACTACATGGTGGTGGCCACCGAC | |
| | CAGCAGACCTCTAGGCAGGCCCAGGCCCAGCTGCTTGTAACAGTGG | |
| | AGGGGTCATATGTGGCCGAGGAGGCGGGCTGCCCCCTGTCCTGTGC | |
| | AGTCAGCAAGAGACGGCTGGAGTGTGAGGAGTGTGGCGGCCTGGG | |
| | CTCCCCAACAGGCAGGTGTGAGTGGAGGCAAGGAGATGGCAAAGG | |
| | GATCACCAGGAACTTCTCCACCTGCTCTCCCAGCACCAAGACCTGC | |
| | CCCGACGGCCACTGCGATGTTGTGGAGACCCAAGACATCAACATTT | |
| | GCCCTCAGGACTGCCTCCGGGGCAGCATTGTTGGGGGACACGAGCC | |
| | TGGGGAGCCCCGGGGGATTAAAGCTGGCTATGGCACCTGCAACTGC | |
| | TTCCCTGAGGAGGAGAAGTGCTTCTGCGAGCCCGAAGACATCCAGG | |
| | ATCCACTGTGCGACGAGCTGTGCCGCACGGTGATCGCAGCCGCTGT | |
| | CCTCTTCTCCTTCATCGTCTCGGTGCTGCTGTCTGCCTTCTGCATCCA | |
| | CTGCTACCACAAGTTTGCCCACAAGCCACCCATCTCCTCAGCTGAG | |
| | ATGACCTTCCGGAGGCCCGCCCAGGCCTTCCCGGTCAGCTACTCCT | |
| | CTTCCGGTGCCCGCCGGCCCTCGCTGGACTCCATGGAGAACCAGGT | |
| | CTCCGTGGATGCCTTCAAGATCCTGGAGGATCCAAAGTGGGAATTC | |
| | CCTCGGAAGAACTTGGTTCTTGGAAAAACTCTAGGAGAAGGCGAAT | |
| | TTGGAAAAGTGGTCAAGGCAACGGCCTTCCATCTGAAAGGCAGAG | |
| | CAGGGTACACCACGGTGGCCGTGAAGATGCTGAAAGAGAACGCCT | |
| | CCCCGAGTGAGCTGCGAGACCTGCTGTCAGAGTTCAACGTCCTGAA | |
| | GCAGGTCAACCACCCACATGTCATCAAATTGTATGGGGCCTGCAGC | |
| | CAGGATGGCCCGCTCCTCCTCATCGTGGAGTACGCCAAATACGGCT | |
| | CCCTGCGGGGCTTCCTCCGCGAGAGCCGCAAAGTGGGGCCTGGCTA | |
| | CCTGGGCAGTGGAGGCAGCCGCAACTCCAGCTCCCTGGACCACCCG | |
| | GATGAGCGGGCCCTCACCATGGGCGACCTCATCTCATTTGCCTGGC | |
| | AGATCTCACAGGGGATGCAGTATCTGGCCGAGATGAAGCTCGTTCA | |
| | TCGGGACTTGGCAGCCAGAAACATCCTGGTAGCTGAGGGGCGGAA | |
| | GATGAAGATTTCGGATTTCGGCTTGTCCCGAGATGTTTATGAAGAG | |
| | GATTCCTACGTGAAGAGGAGCCAGGGTCGGATTCCAGTTAAATGGA | |
| | TGGCAATTGAATCCCTTTTTGATCATATCTACACCACGCAAAGTGAT | |
| | GTATGGTCTTTTGGTGTCCTGCTGTGGGAGATCGTGACCCTAGGGG | |
| | GAAACCCCTATCCTGGGATTCCTCCTGAGCGGCTCTTCAACCTTCTG | |
| | AAGACCGGCCACCGGATGGAGAGGCCAGACAACTGCAGCGAGGAG | |
| | ATGTACCGCCTGATGCTGCAATGCTGGAAGCAGGAGCCGGACAAA | |
| | AGGCCGGTGTTTGCGGACATCAGCAAAGACCTGGAGAAGATGATG | |
| | GTTAAGAGGAGAGACTACTTGGACCTTGCGGCGTCCACTCCATCTG | |
| | ACTCCCTGATTTATGACGACGGCCTCTCAGAGGAGGAGACACCGCT | |
| | GGTGGACTGTAATAATGCCCCCCTCCCTCGAGCCCTCCCTTCCACAT | |
| | GGATTGAAAACAAACTCTATGGTAGAATTTCCCATGCATTTACTAG | |
| | ATTCTAG | |
| PIBF1- | ATGTCTCGAAAAATTTCAAAGGAGTCAAAAAAAGTGAACATCTCTA | 152 |
| RET | GTTCTCTGGAATCTGAAGATATTAGTTTAGAAACAACAGTTCCTAC | |
| | GGATGATATTTCCTCATCAGAAGAGCGAGAGGGCAAAGTCAGAAT | |
| | CACCAGGCAGCTAATTGAACGAAAAGAACTACTTCATAATATTCAG | |
| | TTACTAAAAATTGAGCTATCCCAGAAAACTATGATGATCGACAATT | |
| | TGAAAGTGGATTATCTTACAAAGATTGAAGAATTGGAGGAGAAACT | |
| | TAATGATGCACTTCACCAGAAGCAGCTACTAACATTGAGATTAGAC | |
| | AACCAATTGGCTTTTCAACAGAAAGATGCCAGCAAATATCAAGAAT | |
| | TAATGAAACAAGAAATGGAAACCATTTTGTTGAGACAGAAACAAC | |
| | TAGAAGAGACAAATCTTCAGCTAAGAGAAAAAGCTGGAGATGTTC | |
| | GTCGAAACCTGCGTGACTTTGAGTTGACAGAAGAGCAATATATTAA | |
| | ATTAAAAGCTTTTCCTGAAGATCAGCTTTCTATTCCTGAATATGTAT | |
| | CTGTTCGCTTCTATGAGCTAGTGAATCCATTAAGAAAGGAAATCTG | |
| | TGAACTACAAGTGAAAAAGAATATCCTAGCAGAAGAATTAAGTAC | |
| | AAACAAAAACCAACTGAAGCAGCTGACAGAGACATATGAGGAAGA | |
| | TCGAAAAAACTACTCTGAAGTTCAAATTAGATGTCAACGTTTGGCC | |
| | TTAGAATTAGCAGACACAAAACAGTTAATTCAGCAAGGTGACTACC | |
| | GTCAAGAGAACTATGATAAAGTCAAGAGTGAACGTGATGCACTTG | |
| | AACAGGAAGTAATTGAGCTTAGGAGAAAACATGAAATACTTGAAG | |
| | CCTCTCACATGATTCAAACAAAAGAACGAAGTGAATTATCAAAAGA | |
| | GGTAGTCACCTTAGAGCAAACTGTTACTTTACTGCAAAAGGATAAA | |
| | GAATATCTTAATCGCCAAAACATGGAGCTTAGTGTTCGCTGTGCTC | |
| | ATGAAGAGGATCGCCTTGAAAGACTTCAAGCTCAACTGGAAGAAA | |
| | GCAAAAAGGCTAGAGAAGAGATGTATGAAAAATATGTAGCATCCA | |
| | GAGACCATTATAAAACAGAATATGAAAATAAACTACATGATGAAC | |
| | TAGAACAAATCAGATTGAAAACCAACCAAGAAATTGATCAACTTCG | |
| | AAATGCCTCTAGGGAAATGTATGAACGAGAAAACAGAAATCTCCG | |
| | AGAAGCAAGGGATAATGCTGTGGCTGAAAAGGAACGAGCAGTGAT | |
| | GGCTGAAAAGGATGCTTTAGAAAAACACGATCAGCTCTTAGACAG | |
| | GTACAGAGAACTACAACTTAGTACAGAAAGCAAAGTAACAGAATT | |
| | TCTCCATCAAAGTAAATTAAAATCTTTTGAAAGTGAGCGTGTTCAA | |
| | CTTCTGCAAGAGGAAACAGCAAGAAATCTCACACAGTGTCAATTGG | |
| | AATGTGAAAAATATCAGAAAAAATTGGAGGTTTTAACCAAAGAATT | |
| | TTATAGTCTCCAAGCCTCTTCTGAAAAACGCATTACTGAACTTCAAG | |
| | CACAGAACTCAGAGCATCAAGCAAGGCTAGACATTTATGAGAAAC | |
| | TGGAAAAAGAGCTTGATGAAATAATAATGCAAACTGCAGAAATTG | |
| | AAAATGAAGATGAGGCTGAAAGGGTTCTTTTTTCCTACGGCTATGG | |
| | TGCTAATGTTCCCACAACAGCCAAAAGACGACTAAAGCAAAGTGTT | |
| | CACTTGGCAAGAAGAGTGCTTCAATTAGAAAAACAAAACTCGCTGA | |
| | TTTTAAAAGATCTGGAACATCGAAAGGACCAAGTAACACAGCTTTC | |
| | ACAAGAGCTTGACAGAGCCAATTCGCTATTAAACCAGACTCAACAG | |
| | CCTTACAGGTATCTCATTGAATCAGTGCGTCAGAGAGATTCTAAGA | |
| | TTGATTCACTGACGGAATCTATTGCACAACTTGAGAAAGATGTCAG | |
| | CAACTTAAATAAAGAAAAGTCAGCTTTACTACAGACGAAGAATCA | |
| | AATGGCATTAGATTTAGAACAACTTCTAAATCATCGTGAGGAGGAT | |
| | CCAAAGTGGGAATTCCCTCGGAAGAACTTGGTTCTTGGAAAAACTC | |
| | TAGGAGAAGGCGAATTTGGAAAAGTGGTCAAGGCAACGGCCTTCC | |
| | ATCTGAAAGGCAGAGCAGGGTACACCACGGTGGCCGTGAAGATGC | |
| | TGAAAGAGAACGCCTCCCCGAGTGAGCTGCGAGACCTGCTGTCAGA | |
| | GTTCAACGTCCTGAAGCAGGTCAACCACCCACATGTCATCAAATTG | |
| | TATGGGGCCTGCAGCCAGGATGGCCCGCTCCTCCTCATCGTGGAGT | |
| | ACGCCAAATACGGCTCCCTGCGGGGCTTCCTCCGCGAGAGCCGCAA | |
| | AGTGGGGCCTGGCTACCTGGGCAGTGGAGGCAGCCGCAACTCCAG | |
| | CTCCCTGGACCACCCGGATGAGCGGGCCCTCACCATGGGCGACCTC | |
| | ATCTCATTTGCCTGGCAGATCTCACAGGGGATGCAGTATCTGGCCG | |
| | AGATGAAGCTCGTTCATCGGGACTTGGCAGCCAGAAACATCCTGGT | |
| | AGCTGAGGGGCGGAAGATGAAGATTTCGGATTTCGGCTTGTCCCGA | |
| | GATGTTTATGAAGAGGATTCCTACGTGAAGAGGAGCCAGGGTCGG | |
| | ATTCCAGTTAAATGGATGGCAATTGAATCCCTTTTTGATCATATCTA | |
| | CACCACGCAAAGTGATGTATGGTCTTTTGGTGTCCTGCTGTGGGAG | |
| | ATCGTGACCCTAGGGGGAAACCCCTATCCTGGGATTCCTCCTGAGC | |
| | GGCTCTTCAACCTTCTGAAGACCGGCCACCGGATGGAGAGGCCAGA | |
| | CAACTGCAGCGAGGAGATGTACCGCCTGATGCTGCAATGCTGGAAG | |
| | CAGGAGCCGGACAAAAGGCCGGTGTTTGCGGACATCAGCAAAGAC | |
| | CTGGAGAAGATGATGGTTAAGAGGAGAGACTACTTGGACCTTGCG | |
| | GCGTCCACTCCATCTGACTCCCTGATTTATGACGACGGCCTCTCAGA | |
| | GGAGGAGACACCGCTGGTGGACTGTAATAATGCCCCCCTCCCTCGA | |
| | GCCCTCCCTTCCACATGGATTGAAAACAAACTCTATGGTAGAATTT | |
| | CCCATGCATTTACTAGATTCTAG | |
| PIBF1- | ATGTCTCGAAAAATTTCAAAGGAGTCAAAAAAAGTGAACATCTCTA | 153 |
| RET | GTTCTCTGGAATCTGAAGATATTAGTTTAGAAACAACAGTTCCTAC | |
| | GGATGATATTTCCTCATCAGAAGAGCGAGAGGGCAAAGTCAGAAT | |
| | CACCAGGCAGCTAATTGAACGAAAAGAACTACTTCATAATATTCAG | |
| | TTACTAAAAATTGAGCTATCCCAGAAAACTATGATGATCGACAATT | |
| | TGAAAGTGGATTATCTTACAAAGATTGAAGAATTGGAGGAGAAACT | |
| | TAATGATGCACTTCACCAGAAGCAGCTACTAACATTGAGATTAGAC | |
| | AACCAATTGGCTTTTCAACAGAAAGATGCCAGCAAATATCAAGAAT | |
| | TAATGAAACAAGAAATGGAAACCATTTTGTTGAGACAGAAACAAC | |
| | TAGAAGAGACAAATCTTCAGCTAAGAGAAAAAGCTGGAGATGTTC | |
| | GTCGAAACCTGCGTGACTTTGAGTTGACAGAAGAGCAATATATTAA | |
| | ATTAAAAGCTTTTCCTGAAGATCAGCTTTCTATTCCTGAATATGTAT | |
| | CTGTTCGCTTCTATGAGCTAGTGAATCCATTAAGAAAGGAAATCTG | |
| | TGAACTACAAGTGAAAAAGAATATCCTAGCAGAAGAATTAAGTAC | |
| | AAACAAAAACCAACTGAAGCAGCTGACAGAGACATATGAGGAAGA | |
| | TCGAAAAAACTACTCTGAAGTTCAAATTAGATGTCAACGTTTGGCC | |
| | TTAGAATTAGCAGACACAAAACAGTTAATTCAGCAAGGTGACTACC | |
| | GTCAAGAGAACTATGATAAAGTCAAGAGTGAACGTGATGCACTTG | |
| | AACAGGAAGTAATTGAGCTTAGGAGAAAACATGAAATACTTGAAG | |
| | CCTCTCACATGATTCAAACAAAAGAACGAAGTGAATTATCAAAAGA | |
| | GGTAGTCACCTTAGAGCAAACTGTTACTTTACTGCAAAAGGATAAA | |
| | GAATATCTTAATCGCCAAAACATGGAGCTTAGTGTTCGCTGTGCTC | |
| | ATGAAGAGGATCGCCTTGAAAGACTTCAAGCTCAACTGGAAGAAA | |
| | GCAAAAAGGCTAGAGAAGAGATGTATGAAAAATATGTAGCATCCA | |
| | GAGACCATTATAAAACAGAATATGAAAATAAACTACATGATGAAC | |
| | TAGAACAAATCAGATTGAAAACCAACCAAGAAATTGATCAACTTCG | |
| | AAATGCCTCTAGGGAAATGTATGAACGAGAAAACAGAAATCTCCG | |
| | AGAAGCAAGGGATAATGCTGTGGCTGAAAAGGAACGAGCAGTGAT | |
| | GGCTGAAAAGGATGCTTTAGAAAAACACGATCAGCTCTTAGACAG | |
| | GTACAGAGAACTACAACTTAGTACAGAAAGCAAAGTAACAGAATT | |
| | TCTCCATCAAAGTAAATTAAAATCTTTTGAAAGTGAGCGTGTTCAA | |
| | CTTCTGCAAGAGGAAACAGCAAGAAATCTCACACAGTGTCAATTGG | |
| | AATGTGAAAAATATCAGAAAAAATTGGAGGTTTTAACCAAAGAATT | |
| | TTATAGTCTCCAAGCCTCTTCTGAAAAACGCATTACTGAACTTCAAG | |
| | CACAGAACTCAGAGCATCAAGCAAGGCTAGACATTTATGAGAAAC | |
| | TGGAAAAAGAGCTTGATGAAATAATAATGCAAACTGCAGAAATTG | |
| | AAAATGAAGATGAGGCTGAAAGGGTTCTTTTTTCCTACGGCTATGG | |
| | TGCTAATGTTCCCACAACAGCCAAAAGACGACTAAAGCAAAGTGTT | |
| | CACTTGGCAAGAAGAGTGCTTCAATTAGAAAAACAAAACTCGCTGA | |
| | TTTTAAAAGATCTGGAACATCGAAAGGACCAAGTAACACAGCTTTC | |
| | ACAAGAGCTTGACAGAGCCAATTCGCTATTAAACCAGACTCAACAG | |
| | CCTTACAGGTATCTCATTGAATCAGTGCGTCAGAGAGATTCTAAGA | |
| | TTGATTCACTGACGGAATCTATTGCACAACTTGAGAAAGATGTCAG | |
| | CAACTTAAATAAAGAAAAGTCAGCTTTACTACAGACGAAGAATCA | |
| | AATGGCATTAGATTTAGAACAACTTCTAAATCATCGTGAGGAGGAT | |
| | CCAAAGTGGGAATTCCCTCGGAAGAACTTGGTTCTTGGAAAAACTC | |
| | TAGGAGAAGGCGAATTTGGAAAAGTGGTCAAGGCAACGGCCTTCC | |
| | ATCTGAAAGGCAGAGCAGGGTACACCACGGTGGCCGTGAAGATGC | |
| | TGAAAGAGAACGCCTCCCCGAGTGAGCTGCGAGACCTGCTGTCAGA | |
| | GTTCAACGTCCTGAAGCAGGTCAACCACCCACATGTCATCAAATTG | |
| | TATGGGGCCTGCAGCCAGGATGGCCCGCTCCTCCTCATCGTGGAGT | |
| | ACGCCAAATACGGCTCCCTGCGGGGCTTCCTCCGCGAGAGCCGCAA | |
| | AGTGGGGCCTGGCTACCTGGGCAGTGGAGGCAGCCGCAACTCCAG | |
| | CTCCCTGGACCACCCGGATGAGCGGGCCCTCACCATGGGCGACCTC | |
| | ATCTCATTTGCCTGGCAGATCTCACAGGGGATGCAGTATCTGGCCG | |
| | AGATGAAGCTCGTTCATCGGGACTTGGCAGCCAGAAACATCCTGGT | |
| | AGCTGAGGGGCGGAAGATGAAGATTTCGGATTTCGGCTTGTCCCGA | |
| | GATGTTTATGAAGAGGATTCCTACGTGAAGAGGAGCCAGGGTCGG | |
| | ATTCCAGTTAAATGGATGGCAATTGAATCCCTTTTTGATCATATCTA | |
| | CACCACGCAAAGTGATGTATGGTCTTTTGGTGTCCTGCTGTGGGAG | |
| | ATCGTGACCCTAGGGGGAAACCCCTATCCTGGGATTCCTCCTGAGC | |
| | GGCTCTTCAACCTTCTGAAGACCGGCCACCGGATGGAGAGGCCAGA | |
| | CAACTGCAGCGAGGAGATGTACCGCCTGATGCTGCAATGCTGGAAG | |
| | CAGGAGCCGGACAAAAGGCCGGTGTTTGCGGACATCAGCAAAGAC | |
| | CTGGAGAAGATGATGGTTAAGAGGAGAGACTACTTGGACCTTGCG | |
| | GCGTCCACTCCATCTGACTCCCTGATTTATGACGACGGCCTCTCAGA | |
| | GGAGGAGACACCGCTGGTGGACTGTAATAATGCCCCCCTCCCTCGA | |
| | GCCCTCCCTTCCACATGGATTGAAAACAAACTCTATGGTAGAATTT | |
| | CCCATGCATTTACTAGATTCTAG | |
| SLC12A2- | ATGGAGCCGCGGCCCACGGCGCCCTCCTCCGGCGCCCCGGGACTGG | 154 |
| RET | CCGGGGTCGGGGAGACGCCGTCAGCCGCTGCGCTGGCCGCAGCCA | |
| | GGGTGGAACTGCCCGGCACGGCTGTGCCCTCGGTGCCGGAGGATGC | |
| | TGCGCCCGCGAGCCGGGACGGCGGCGGGGTCCGCGATGAGGGCCC | |
| | CGCGGCGGCCGGGGACGGGCTGGGCAGACCCTTGGGGCCCACCCC | |
| | GAGCCAGAGCCGTTTCCAGGTGGACCTGGTTTCCGAGAACGCCGGG | |
| | CGGGCCGCTGCTGCGGCGGCGGCGGCGGCGGCGGCAGCGGCGGCG | |
| | GCTGGTGCTGGGGCGGGGGCCAAGCAGACCCCCGCGGACGGGGAA | |
| | GCCAGCGGCGAGAGCGAGCCGGCTAAAGGCAGCGAGGAAGCCAAG | |
| | GGCCGCTTCCGCGTGAACTTCGTGGACCCAGCTGCCTCCTCGTCGG | |
| | CTGAAGACAGCCTGTCAGATGCTGCCGGGGTCGGAGTCGACGGGCC | |
| | CAACGTGAGCTTCCAGAACGGCGGGGACACGGTGCTGAGCGAGGG | |
| | CAGCAGCCTGCACTCCGGCGGCGGCGGCGGCAGTGGGCACCACCA | |
| | GCACTACTATTATGATACCCACACCAACACCTACTACCTGCGCACC | |
| | TTCGGCCACAACACCATGGACGCTGTGCCCAGGATCGATCACTACC | |
| | GGCACACAGCCGCGCAGCTGGGCGAGAAGCTGCTCCGGCCTAGCCT | |
| | GGCGGAGCTCCACGACGAGCTGGAAAAGGAACCTTTTGAGGATGG | |
| | CTTTGCAAATGGGGAAGAAAGTACTCCAACCAGAGATGCTGTGGTC | |
| | ACGTATACTGCAGAAAGTAAAGGAGTCGTGAAGTTTGGCTGGATCA | |
| | AGGGTGTATTAGTACGTTGTATGTTAAACATTTGGGGTGTGATGCTT | |
| | TTCATTAGATTGTCATGGATTGTGGGTCAAGCTGGAATAGGTCTATC | |
| | AGTCCTTGTAATAATGATGGCCACTGTTGTGACAACTATCACAGGA | |
| | TTGTCTACTTCAGCAATAGCAACTAATGGATTTGTAAGAGGAGGAG | |
| | GAGCATATTATTTAATATCTAGAAGTCTAGGGCCAGAATTTGGTGG | |
| | TGCAATTGGTCTAATCTTCGCCTTTGCCAACGCTGTTGCAGTTGCTA | |
| | TGTATGTGGTTGGATTTGCAGAAACCGTGGTGGAGTTGCTTAAGGA | |
| | ACATTCCATACTTATGATAGATGAAATCAATGATATCCGAATTATT | |
| | GGAGCCATTACAGTCGTGATTCTTTTAGGTATCTCAGTAGCTGGAAT | |
| | GGAGTGGGAAGCAAAAGCTCAGATTGTTCTTTTGGTGATCCTACTT | |
| | CTTGCTATTGGTGATTTCGTCATAGGAACATTTATCCCACTGGAGAG | |
| | CAAGAAGCCAAAAGGGTTTTTTGGTTATAAATCTGAAATATTTAAT | |
| | GAGAACTTTGGGCCCGATTTTCGAGAGGAAGAGACTTTCTTTTCTGT | |
| | ATTTGCCATCTTTTTTCCTGCTGCAACTGGTATTCTGGCTGGAGCAA | |
| | ATATCTCAGGTGATCTTGCAGATCCTCAGTCAGCCATACCCAAAGG | |
| | AACACTCCTAGCCATTTTAATTACTACATTGGTTTACGTAGGAATTG | |
| | CAGTATCTGTAGGTTCTTGTGTTGTTCGAGATGCCACTGGAAACGTT | |
| | AATGACACTATCGTAACAGAGCTAACAAACTGTACTTCTGCAGCCT | |
| | GCAAATTAAACTTTGATTTTTCATCTTGTGAAAGCAGTCCTTGTTCC | |
| | TATGGCCTAATGAACAACTTCCAGGTAATGAGTATGGTGTCAGGAT | |
| | TTACACCACTAATTTCTGCAGGTATATTTTCAGCCACTCTTTCTTCA | |
| | GCATTAGCATCCCTAGTGAGTGCTCCCAAAATATTTCAGGCTCTATG | |
| | TAAGGACAACATCTACCCAGCTTTCCAGATGTTTGCTAAAGGTTAT | |
| | GGGAAAAATAATGAACCTCTTCGTGGCTACATCTTAACATTCTTAA | |
| | TTGCACTTGGATTCATCTTAATTGCTGAACTGAATGTTATTGCACCA | |
| | ATTATCTCAAACTTCTTCCTTGCATCATATGCATTGATCAATTTTTCA | |
| | GTATTCCATGCATCACTTGCAAAATCTCCAGGATGGCGTCCTGCATT | |
| | CAAATACTACAACATGTGGATATCACTTCTTGGAGCAATTCTTTGTT | |
| | GCATAGTAATGTTCGTCATTAACTGGTGGGCTGCATTGCTAACATAT | |
| | GTGATAGTCCTTGGGCTGTATATTTATGTTACCTACAAAAAACCAG | |
| | ATGTGAATTGGGGATCCTCTACACAAGCCCTGACTTACCTGAATGC | |
| | ACTGCAGCATTCAATTCGTCTTTCTGGAGTGGAAGACCACGTGAAA | |
| | AACTTTAGGCCACAGTGTCTTGTTATGACAGGTGCTCCAAACTCAC | |
| | GTCCAGCTTTACTTCATCTTGTTCATGATTTCACAAAAAATGTTGGT | |
| | TTGATGATCTGTGGCCATGTACATATGGAGGATCCAAAGTGGGAAT | |
| | TCCCTCGGAAGAACTTGGTTCTTGGAAAAACTCTAGGAGAAGGCGA | |
| | ATTTGGAAAAGTGGTCAAGGCAACGGCCTTCCATCTGAAAGGCAGA | |
| | GCAGGGTACACCACGGTGGCCGTGAAGATGCTGAAAGAGAACGCC | |
| | TCCCCGAGTGAGCTGCGAGACCTGCTGTCAGAGTTCAACGTCCTGA | |
| | AGCAGGTCAACCACCCACATGTCATCAAATTGTATGGGGCCTGCAG | |
| | CCAGGATGGCCCGCTCCTCCTCATCGTGGAGTACGCCAAATACGGC | |
| | TCCCTGCGGGGCTTCCTCCGCGAGAGCCGCAAAGTGGGGCCTGGCT | |
| | ACCTGGGCAGTGGAGGCAGCCGCAACTCCAGCTCCCTGGACCACCC | |
| | GGATGAGCGGGCCCTCACCATGGGCGACCTCATCTCATTTGCCTGG | |
| | CAGATCTCACAGGGGATGCAGTATCTGGCCGAGATGAAGCTCGTTC | |
| | ATCGGGACTTGGCAGCCAGAAACATCCTGGTAGCTGAGGGGCGGA | |
| | AGATGAAGATTTCGGATTTCGGCTTGTCCCGAGATGTTTATGAAGA | |
| | GGATTCCTACGTGAAGAGGAGCCAGGGTCGGATTCCAGTTAAATGG | |
| | ATGGCAATTGAATCCCTTTTTGATCATATCTACACCACGCAAAGTG | |
| | ATGTATGGTCTTTTGGTGTCCTGCTGTGGGAGATCGTGACCCTAGGG | |
| | GGAAACCCCTATCCTGGGATTCCTCCTGAGCGGCTCTTCAACCTTCT | |
| | GAAGACCGGCCACCGGATGGAGAGGCCAGACAACTGCAGCGAGGA | |
| | GATGTACCGCCTGATGCTGCAATGCTGGAAGCAGGAGCCGGACAA | |
| | AAGGCCGGTGTTTGCGGACATCAGCAAAGACCTGGAGAAGATGAT | |
| | GGTTAAGAGGAGAGACTACTTGGACCTTGCGGCGTCCACTCCATCT | |
| | GACTCCCTGATTTATGACGACGGCCTCTCAGAGGAGGAGACACCGC | |
| | TGGTGGACTGTAATAATGCCCCCCTCCCTCGAGCCCTCCCTTCCACA | |
| | TGGATTGAAAACAAACTCTATGGTAGAATTTCCCATGCATTTACTA | |
| | GATTCTAG | |
| ZNF485 | ATGGCCCCAAGAGCCCAGATCCAGGGACCGCTGACATTTGGGGATG | 155 |
| RET | TGGCTGTGGCCTTTACCCGGATTGAGTGGAGACACCTGGATGCTGC | |
| | TCAGCGGGCCCTGTACAGGGATGTGATGCTGGAGAACTATGGGAAT | |
| | CTGGTGTCTGTGGGGCTTCTCTCTTCCAAACCAAAACTAATTACTCA | |
| | GTTGGAGCAAGGGGCAGAGCCCTGGACTGAGGTGCGAGAGGCTCC | |
| | ATCAGGCACACATGCAGGAGGATCCAAAGTGGGAATTCCCTCGGA | |
| | AGAACTTGGTTCTTGGAAAAACTCTAGGAGAAGGCGAATTTGGAAA | |
| | AGTGGTCAAGGCAACGGCCTTCCATCTGAAAGGCAGAGCAGGGTA | |
| | CACCACGGTGGCCGTGAAGATGCTGAAAGAGAACGCCTCCCCGAG | |
| | TGAGCTGCGAGACCTGCTGTCAGAGTTCAACGTCCTGAAGCAGGTC | |
| | AACCACCCACATGTCATCAAATTGTATGGGGCCTGCAGCCAGGATG | |
| | GCCCGCTCCTCCTCATCGTGGAGTACGCCAAATACGGCTCCCTGCG | |
| | GGGCTTCCTCCGCGAGAGCCGCAAAGTGGGGCCTGGCTACCTGGGC | |
| | AGTGGAGGCAGCCGCAACTCCAGCTCCCTGGACCACCCGGATGAGC | |
| | GGGCCCTCACCATGGGCGACCTCATCTCATTTGCCTGGCAGATCTC | |
| | ACAGGGGATGCAGTATCTGGCCGAGATGAAGCTCGTTCATCGGGAC | |
| | TTGGCAGCCAGAAACATCCTGGTAGCTGAGGGGCGGAAGATGAAG | |
| | ATTTCGGATTTCGGCTTGTCCCGAGATGTTTATGAAGAGGATTCCTA | |
| | CGTGAAGAGGAGCCAGGGTCGGATTCCAGTTAAATGGATGGCAATT | |
| | GAATCCCTTTTTGATCATATCTACACCACGCAAAGTGATGTATGGTC | |
| | TTTTGGTGTCCTGCTGTGGGAGATCGTGACCCTAGGGGGAAACCCC | |
| | TATCCTGGGATTCCTCCTGAGCGGCTCTTCAACCTTCTGAAGACCGG | |
| | CCACCGGATGGAGAGGCCAGACAACTGCAGCGAGGAGATGTACCG | |
| | CCTGATGCTGCAATGCTGGAAGCAGGAGCCGGACAAAAGGCCGGT | |
| | GTTTGCGGACATCAGCAAAGACCTGGAGAAGATGATGGTTAAGAG | |
| | GAGAGACTACTTGGACCTTGCGGCGTCCACTCCATCTGACTCCCTG | |
| | ATTTATGACGACGGCCTCTCAGAGGAGGAGACACCGCTGGTGGACT | |
| | GTAATAATGCCCCCCTCCCTCGAGCCCTCCCTTCCACATGGATTGAA | |
| | AACAAACTCTATGGTAGAATTTCCCATGCATTTACTAGATTCTAG | |
| ZNF485- | ATGGCCCCAAGAGCCCAGATCCAGGGACCGCTGACATTTGGGGATG | 156 |
| RET | TGGCTGTGGCCTTTACCCGGATTGAGTGGAGACACCTGGATGCTGC | |
| | TCAGCGGGCCCTGTACAGGGATGTGATGCTGGAGAACTATGGGAAT | |
| | CTGGTGTCTGTGGGGCTTCTCTCTTCCAAACCAAAACTAATTACTCA | |
| | GTTGGAGCAAGGGGCAGAGCCCTGGACTGAGGTGCGAGAGGCTCC | |
| | ATCAGGCACACATGCAGGGGGCAGCATTGTTGGGGGACACGAGCC | |
| | TGGGGAGCCCCGGGGGATTAAAGCTGGCTATGGCACCTGCAACTGC | |
| | TTCCCTGAGGAGGAGAAGTGCTTCTGCGAGCCCGAAGACATCCAGG | |
| | ATCCACTGTGCGACGAGCTGTGCCGCACGGTGATCGCAGCCGCTGT | |
| | CCTCTTCTCCTTCATCGTCTCGGTGCTGCTGTCTGCCTTCTGCATCCA | |
| | CTGCTACCACAAGTTTGCCCACAAGCCACCCATCTCCTCAGCTGAG | |
| | ATGACCTTCCGGAGGCCCGCCCAGGCCTTCCCGGTCAGCTACTCCT | |
| | CTTCCGGTGCCCGCCGGCCCTCGCTGGACTCCATGGAGAACCAGGT | |
| | CTCCGTGGATGCCTTCAAGATCCTGGAGGATCCAAAGTGGGAATTC | |
| | CCTCGGAAGAACTTGGTTCTTGGAAAAACTCTAGGAGAAGGCGAAT | |
| | TTGGAAAAGTGGTCAAGGCAACGGCCTTCCATCTGAAAGGCAGAG | |
| | CAGGGTACACCACGGTGGCCGTGAAGATGCTGAAAGAGAACGCCT | |
| | CCCCGAGTGAGCTGCGAGACCTGCTGTCAGAGTTCAACGTCCTGAA | |
| | GCAGGTCAACCACCCACATGTCATCAAATTGTATGGGGCCTGCAGC | |
| | CAGGATGGCCCGCTCCTCCTCATCGTGGAGTACGCCAAATACGGCT | |
| | CCCTGCGGGGCTTCCTCCGCGAGAGCCGCAAAGTGGGGCCTGGCTA | |
| | CCTGGGCAGTGGAGGCAGCCGCAACTCCAGCTCCCTGGACCACCCG | |
| | GATGAGCGGGCCCTCACCATGGGCGACCTCATCTCATTTGCCTGGC | |
| | AGATCTCACAGGGGATGCAGTATCTGGCCGAGATGAAGCTCGTTCA | |
| | TCGGGACTTGGCAGCCAGAAACATCCTGGTAGCTGAGGGGCGGAA | |
| | GATGAAGATTTCGGATTTCGGCTTGTCCCGAGATGTTTATGAAGAG | |
| | GATTCCTACGTGAAGAGGAGCCAGGGTCGGATTCCAGTTAAATGGA | |
| | TGGCAATTGAATCCCTTTTTGATCATATCTACACCACGCAAAGTGAT | |
| | GTATGGTCTTTTGGTGTCCTGCTGTGGGAGATCGTGACCCTAGGGG | |
| | GAAACCCCTATCCTGGGATTCCTCCTGAGCGGCTCTTCAACCTTCTG | |
| | AAGACCGGCCACCGGATGGAGAGGCCAGACAACTGCAGCGAGGAG | |
| | ATGTACCGCCTGATGCTGCAATGCTGGAAGCAGGAGCCGGACAAA | |
| | AGGCCGGTGTTTGCGGACATCAGCAAAGACCTGGAGAAGATGATG | |
| | GTTAAGAGGAGAGACTACTTGGACCTTGCGGCGTCCACTCCATCTG | |
| | ACTCCCTGATTTATGACGACGGCCTCTCAGAGGAGGAGACACCGCT | |
| | GGTGGACTGTAATAATGCCCCCCTCCCTCGAGCCCTCCCTTCCACAT | |
| | GGATTGAAAACAAACTCTATGGTAGAATTTCCCATGCATTTACTAG | |
| | ATTCTAG | |

In some embodiments, the GOLGB1-RET fusion nucleic acid molecule of the disclosure comprises the nucleotide sequence of SEQ ID NO: 2, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the GP2-RET fusion nucleic acid molecule of the disclosure comprises the nucleotide sequence of SEQ ID NO: 3, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the PPP2R5A-RET fusion nucleic acid molecule of the disclosure comprises the nucleotide sequence of SEQ ID NO: 4, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the GRM7-RET fusion nucleic acid molecule of the disclosure comprises the nucleotide sequence of SEQ ID NO: 6, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the MYH14-RET fusion nucleic acid molecule of the disclosure comprises the nucleotide sequence of SEQ ID NO: 8, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the MGEA5-RET fusion nucleic acid molecule of the disclosure comprises the nucleotide sequence of SEQ ID NO: 9, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the SAMD4A-RET fusion nucleic acid molecule of the disclosure comprises the nucleotide sequence of SEQ ID NO: 10, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ACPP-RET fusion nucleic acid molecule of the disclosure comprises the nucleotide sequence of SEQ ID NO: 11, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the BMS1-RET fusion nucleic acid molecule of the disclosure comprises the nucleotide sequence of SEQ ID NO: 12, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the CEP135-RET fusion nucleic acid molecule of the disclosure comprises the nucleotide sequence of SEQ ID NO: 13, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the EEA1-RET fusion nucleic acid molecule of the disclosure comprises the nucleotide sequence of SEQ ID NO: 14, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the CSGALNACT2-RET fusion nucleic acid molecule of the disclosure comprises the nucleotide sequence of SEQ ID NO: 15, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the KIAA1217-RET fusion nucleic acid molecule of the disclosure comprises the nucleotide sequence of SEQ ID NO: 16, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the SORBS1-RET fusion nucleic acid molecule of the disclosure comprises the nucleotide sequence of SEQ ID NO: 17, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the SORBS1-RET fusion nucleic acid molecule of the disclosure comprises the nucleotide sequence of SEQ ID NO: 18, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the MPRIP-RET fusion nucleic acid molecule of the disclosure comprises the nucleotide sequence of SEQ ID NO: 19, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the TFG-RET fusion nucleic acid molecule of the disclosure comprises the nucleotide sequence of SEQ ID NO: 20, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the SPECC1L-RET fusion nucleic acid molecule of the disclosure comprises the nucleotide sequence of SEQ ID NO: 21, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the REEP3-RET fusion nucleic acid molecule of the disclosure comprises the nucleotide sequence of SEQ ID NO: 22, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RRBP1-RET fusion nucleic acid molecule of the disclosure comprises the nucleotide sequence of SEQ ID NO: 23, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ETV6-RET fusion nucleic acid molecule of the disclosure comprises the nucleotide sequence of SEQ ID NO: 24, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the TAF3-RET fusion nucleic acid molecule of the disclosure comprises the nucleotide sequence of SEQ ID NO: 25, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the PCM1-RET fusion nucleic acid molecule of the disclosure comprises the nucleotide sequence of SEQ ID NO: 26, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the PCM1-RET fusion nucleic acid molecule of the disclosure comprises the nucleotide sequence of SEQ ID NO: 27, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the TNIP2-RET fusion nucleic acid molecule of the disclosure comprises the nucleotide sequence of SEQ ID NO: 28, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the SATB1-RET fusion nucleic acid molecule of the disclosure comprises the nucleotide sequence of SEQ ID NO: 29, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET-ADCY1 fusion nucleic acid molecule of the disclosure comprises the nucleotide sequence of SEQ ID NO: 30, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET-ZNF248 fusion nucleic acid molecule of the disclosure comprises the nucleotide sequence of SEQ ID NO: 31, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET-AGBL4 fusion nucleic acid molecule of the disclosure comprises the nucleotide sequence of SEQ ID NO: 32, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET-LRMDA fusion nucleic acid molecule of the disclosure comprises the nucleotide sequence of SEQ ID NO: 33, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET-ARHGAP19 fusion nucleic acid molecule of the disclosure comprises the nucleotide sequence of SEQ ID NO: 34, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET-CPEB3 fusion nucleic acid molecule of the disclosure comprises the nucleotide sequence of SEQ ID NO: 35, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET-DCC fusion nucleic acid molecule of the disclosure comprises the nucleotide sequence of SEQ ID NO: 36, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET-ELMO1 fusion nucleic acid molecule of the disclosure comprises the nucleotide sequence of SEQ ID NO: 37, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET-WDFY4 fusion nucleic acid molecule of the disclosure comprises the nucleotide sequence of SEQ ID NO: 38, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ANKRD26-RET fusion nucleic acid molecule of the disclosure comprises the nucleotide sequence of SEQ ID NO: 39, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the CLIP1-RET fusion nucleic acid molecule of the disclosure comprises the nucleotide sequence of SEQ ID NO: 40, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the DLG5-RET fusion nucleic acid molecule of the disclosure comprises the nucleotide sequence of SEQ ID NO: 41, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the DLG5-RET fusion nucleic acid molecule of the disclosure comprises the nucleotide sequence of SEQ ID NO: 42, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ERC1-RET fusion nucleic acid molecule of the disclosure comprises the nucleotide sequence of SEQ ID NO: 43, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ERC1-RET fusion nucleic acid molecule of the disclosure comprises the nucleotide sequence of SEQ ID NO: 44, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ERC1-RET fusion nucleic acid molecule of the disclosure comprises the nucleotide sequence of SEQ ID NO: 45, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the FRMD4A-RET fusion nucleic acid molecule of the disclosure comprises the nucleotide sequence of SEQ ID NO: 46, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the KIAA1468-RET fusion nucleic acid molecule of the disclosure comprises the nucleotide sequence of SEQ ID NO: 47, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the NCOA4-RET fusion nucleic acid molecule of the disclosure comprises the nucleotide sequence of SEQ ID NO: 48, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the PARD3-RET fusion nucleic acid molecule of the disclosure comprises the nucleotide sequence of SEQ ID NO: 49, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the PARD3-RET fusion nucleic acid molecule of the disclosure comprises the nucleotide sequence of SEQ ID NO: 50, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the PRKAR1A-RET fusion nucleic acid molecule of the disclosure comprises the nucleotide sequence of SEQ ID NO: 51, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the PRKG1-RET fusion nucleic acid molecule of the disclosure comprises the nucleotide sequence of SEQ ID NO: 52, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the PRKG1-RET fusion nucleic acid molecule of the disclosure comprises the nucleotide sequence of SEQ ID NO: 53, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RUFY2-RET fusion nucleic acid molecule of the disclosure comprises the nucleotide sequence of SEQ ID NO: 54, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the SNRNP70-RET fusion nucleic acid molecule of the disclosure comprises the nucleotide sequence of SEQ ID NO: 55, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the SQSTM1-RET fusion nucleic acid molecule of the disclosure comprises the nucleotide sequence of SEQ ID NO: 56, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the TNIP1-RET fusion nucleic acid molecule of the disclosure comprises the nucleotide sequence of SEQ ID NO: 57, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the TNIP1-RET fusion nucleic acid molecule of the disclosure comprises the nucleotide sequence of SEQ ID NO: 58, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the TNIP1-RET fusion nucleic acid molecule of the disclosure comprises the nucleotide sequence of SEQ ID NO: 59, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the TRIM27-RET fusion nucleic acid molecule of the disclosure comprises the nucleotide sequence of SEQ ID NO: 60, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the TRIM33-RET fusion nucleic acid molecule of the disclosure comprises the nucleotide sequence of SEQ ID NO: 61, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the CCDC6-RET fusion nucleic acid molecule of the disclosure comprises the nucleotide sequence of SEQ ID NO: 62, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the TRIM24-RET fusion nucleic acid molecule of the disclosure comprises the nucleotide sequence of SEQ ID NO: 63, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the FGFR1OP-RET fusion nucleic acid molecule of the disclosure comprises the nucleotide sequence of SEQ ID NO: 64, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the GAS2-RET fusion nucleic acid molecule of the disclosure comprises the nucleotide sequence of SEQ ID NO: 65, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the HOOK1-RET fusion nucleic acid molecule of the disclosure comprises the nucleotide sequence of SEQ ID NO: 66, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the LMNA-RET fusion nucleic acid molecule of the disclosure comprises the nucleotide sequence of SEQ ID NO: 147, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the LMNA-RET fusion nucleic acid molecule of the disclosure comprises the nucleotide sequence of SEQ ID NO: 148, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the MLPH-RET fusion nucleic acid molecule of the disclosure comprises the nucleotide sequence of SEQ ID NO: 149, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the MYH9-RET fusion nucleic acid molecule of the disclosure comprises the nucleotide sequence of SEQ ID NO: 150, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the PCDH15-RET fusion nucleic acid molecule of the disclosure comprises the nucleotide sequence of SEQ ID NO: 151, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the PIBF1-RET fusion nucleic acid molecule of the disclosure comprises the nucleotide sequence of SEQ ID NO: 152, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the PIBF1-RET fusion nucleic acid molecule of the disclosure comprises the nucleotide sequence of SEQ ID NO: 153, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the SLC12A2-RET fusion nucleic acid molecule of the disclosure comprises the nucleotide sequence of SEQ ID NO: 154, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ZNF485-RET fusion nucleic acid molecule of the disclosure comprises the nucleotide sequence of SEQ ID NO: 155, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ZNF485-RET fusion nucleic acid molecule of the disclosure comprises the nucleotide sequence of SEQ ID NO: 156, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto.

In some embodiments, a RET fusion nucleic acid molecule of the disclosure encodes a RET fusion polypeptide comprising the corresponding amino acid sequence as listed in Table 9, below, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the GOLGB1-RET fusion nucleic acid molecule of the disclosure encodes a RET fusion polypeptide comprising the amino acid sequence of SEQ ID NO: 68 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the GP2-RET fusion nucleic acid molecule of the disclosure encodes a RET fusion polypeptide comprising the amino acid sequence of SEQ ID NO: 69 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the PPP2RSA-RET fusion nucleic acid molecule of the disclosure encodes a RET fusion polypeptide comprising the amino acid sequence of SEQ ID NO: 70 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the GRM7-RET fusion nucleic acid molecule of the disclosure encodes a RET fusion polypeptide comprising the amino acid sequence of SEQ ID NO: 72 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the MYH14-RET fusion nucleic acid molecule of the disclosure encodes a RET fusion polypeptide comprising the amino acid sequence of SEQ ID NO: 74 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the MGEA5-RET fusion nucleic acid molecule of the disclosure encodes a RET fusion polypeptide comprising the amino acid sequence of SEQ ID NO: 75 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the SAMD4A-RET fusion nucleic acid molecule of the disclosure encodes a RET fusion polypeptide comprising the amino acid sequence of SEQ ID NO: 76 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ACPP-RET fusion nucleic acid molecule of the disclosure encodes a RET fusion polypeptide comprising the amino acid sequence of SEQ ID NO: 77 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the BMS1-RET fusion nucleic acid molecule of the disclosure encodes a RET fusion polypeptide comprising the amino acid sequence of SEQ ID NO: 78 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the CEP135-RET fusion nucleic acid molecule of the disclosure encodes a RET fusion polypeptide comprising the amino acid sequence of SEQ ID NO: 79 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the EEA1-RET fusion nucleic acid molecule of the disclosure encodes a RET fusion polypeptide comprising the amino acid sequence of SEQ ID NO: 80 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the CSGALNACT2-RET fusion nucleic acid molecule of the disclosure encodes a RET fusion polypeptide comprising the amino acid sequence of SEQ ID NO: 81 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the KIAA1217-RET fusion nucleic acid molecule of the disclosure encodes a RET fusion polypeptide comprising the amino acid sequence of SEQ ID NO: 82 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the SORBS1-RET fusion nucleic acid molecule of the disclosure encodes a RET fusion polypeptide comprising the amino acid sequence of SEQ ID NO: 83 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the SORBS1-RET fusion nucleic acid molecule of the disclosure encodes a RET fusion polypeptide comprising the amino acid sequence of SEQ ID NO: 84 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the MPRIP-RET fusion nucleic acid molecule of the disclosure encodes a RET fusion polypeptide comprising the amino acid sequence of SEQ ID NO: 85 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the TFG-RET fusion nucleic acid molecule of the disclosure encodes a RET fusion polypeptide comprising the amino acid sequence of SEQ ID NO: 86 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the SPECC1L-RET fusion nucleic acid molecule of the disclosure encodes a RET fusion polypeptide comprising the amino acid sequence of SEQ ID NO: 87 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the REEP3-RET fusion nucleic acid molecule of the disclosure encodes a RET fusion polypeptide comprising the amino acid sequence of SEQ ID NO: 88 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RRBP1-RET fusion nucleic acid molecule of the disclosure encodes a RET fusion polypeptide comprising the amino acid sequence of SEQ ID NO: 89 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ETV6-RET fusion nucleic acid molecule of the disclosure encodes a RET fusion polypeptide comprising the amino acid sequence of SEQ ID NO: 90 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the TAF3-RET fusion nucleic acid molecule of the disclosure encodes a RET fusion polypeptide comprising the amino acid sequence of SEQ ID NO: 91 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the PCM1-RET fusion nucleic acid molecule of the disclosure encodes a RET fusion polypeptide comprising the amino acid sequence of SEQ ID NO: 92 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the PCM1-RET fusion nucleic acid molecule of the disclosure encodes a RET fusion polypeptide comprising the amino acid sequence of SEQ ID NO: 93 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the TNIP2-RET fusion nucleic acid molecule of the disclosure encodes a RET fusion polypeptide comprising the amino acid sequence of SEQ ID NO: 94 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the SATB1-RET fusion nucleic acid molecule of the disclosure encodes a RET fusion polypeptide comprising the amino acid sequence of SEQ ID NO: 95 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET-ADCY1 fusion nucleic acid molecule of the disclosure encodes a RET fusion polypeptide comprising the amino acid sequence of SEQ ID NO: 96 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET-ZNF248 fusion nucleic acid molecule of the disclosure encodes a RET fusion polypeptide comprising the amino acid sequence of SEQ ID NO: 97 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET-AGBL4 fusion nucleic acid molecule of the disclosure encodes a RET fusion polypeptide comprising the amino acid sequence of SEQ ID NO: 98 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET-LRMDA fusion nucleic acid molecule of the disclosure encodes a RET fusion polypeptide comprising the amino acid sequence of SEQ ID NO: 99 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET-ARHGAP19 fusion nucleic acid molecule of the disclosure encodes a RET fusion polypeptide comprising the amino acid sequence of SEQ ID NO: 100 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET-CPEB3 fusion nucleic acid molecule of the disclosure encodes a RET fusion polypeptide comprising the amino acid sequence of SEQ ID NO: 101 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET-DCC fusion nucleic acid molecule of the disclosure encodes a RET fusion polypeptide comprising the amino acid sequence of SEQ ID NO: 102 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET-ELMO1 fusion nucleic acid molecule of the disclosure encodes a RET fusion polypeptide comprising the amino acid sequence of SEQ ID NO: 103 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET-WDFY4 fusion nucleic acid molecule of the disclosure encodes a RET fusion polypeptide comprising the amino acid sequence of SEQ ID NO: 104 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ANKRD26-RET fusion nucleic acid molecule of the disclosure encodes a RET fusion polypeptide comprising the amino acid sequence of SEQ ID NO: 105 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the CLIP1-RET fusion nucleic acid molecule of the disclosure encodes a RET fusion polypeptide comprising the amino acid sequence of SEQ ID NO: 106 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the DLG5-RET fusion nucleic acid molecule of the disclosure encodes a RET fusion polypeptide comprising the amino acid sequence of SEQ ID NO: 107 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the DLG5-RET fusion nucleic acid molecule of the disclosure encodes a RET fusion polypeptide comprising the amino acid sequence of SEQ ID NO: 108 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ERC1-RET fusion nucleic acid molecule of the disclosure encodes a RET fusion polypeptide comprising the amino acid sequence of SEQ ID NO: 109 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ERC1-RET fusion nucleic acid molecule of the disclosure encodes a RET fusion polypeptide comprising the amino acid sequence of SEQ ID NO: 110 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ERC1-RET fusion nucleic acid molecule of the disclosure encodes a RET fusion polypeptide comprising the amino acid sequence of SEQ ID NO: 111 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the FRMD4A-RET fusion nucleic acid molecule of the disclosure encodes a RET fusion polypeptide comprising the amino acid sequence of SEQ ID NO: 112 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the KIAA1468-RET fusion nucleic acid molecule of the disclosure encodes a RET fusion polypeptide comprising the amino acid sequence of SEQ ID NO: 113 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the NCOA4-RET fusion nucleic acid molecule of the disclosure encodes a RET fusion polypeptide comprising the amino acid sequence of SEQ ID NO: 114 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the PARD3-RET fusion nucleic acid molecule of the disclosure encodes a RET fusion polypeptide comprising the amino acid sequence of SEQ ID NO: 115 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the PARD3-RET fusion nucleic acid molecule of the disclosure encodes a RET fusion polypeptide comprising the amino acid sequence of SEQ ID NO: 116 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the PRKAR1A-RET fusion nucleic acid molecule of the disclosure encodes a RET fusion polypeptide comprising the amino acid sequence of SEQ ID NO: 117 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the PRKG1-RET fusion nucleic acid molecule of the disclosure encodes a RET fusion polypeptide comprising the amino acid sequence of SEQ ID NO: 118 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the PRKG1-RET fusion nucleic acid molecule of the disclosure encodes a RET fusion polypeptide comprising the amino acid sequence of SEQ ID NO: 119 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RUFY2-RET fusion nucleic acid molecule of the disclosure encodes a RET fusion polypeptide comprising the amino acid sequence of SEQ ID NO: 120 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the SNRNP70-RET fusion nucleic acid molecule of the disclosure encodes a RET fusion polypeptide comprising the amino acid sequence of SEQ ID NO: 121 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the SQSTM1-RET fusion nucleic acid molecule of the disclosure encodes a RET fusion polypeptide comprising the amino acid sequence of SEQ ID NO: 122 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the TNIP1-RET fusion nucleic acid molecule of the disclosure encodes a RET fusion polypeptide comprising the amino acid sequence of SEQ ID NO: 123 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the TNIP1-RET fusion nucleic acid molecule of the disclosure encodes a RET fusion polypeptide comprising the amino acid sequence of SEQ ID NO: 124 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the TNIP1-RET fusion nucleic acid molecule of the disclosure encodes a RET fusion polypeptide comprising the amino acid sequence of SEQ ID NO: 125 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the TRIM27-RET fusion nucleic acid molecule of the disclosure encodes a RET fusion polypeptide comprising the amino acid sequence of SEQ ID NO: 126 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the TRIM33-RET fusion nucleic acid molecule of the disclosure encodes a RET fusion polypeptide comprising the amino acid sequence of SEQ ID NO: 127 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the CCDC6-RET fusion nucleic acid molecule of the disclosure encodes a RET fusion polypeptide comprising the amino acid sequence of SEQ ID NO: 128 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the TRIM24-RET fusion nucleic acid molecule of the disclosure encodes a RET fusion polypeptide comprising the amino acid sequence of SEQ ID NO: 129 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the FGFR1OP-RET fusion nucleic acid molecule of the disclosure encodes a RET fusion polypeptide comprising the amino acid sequence of SEQ ID NO: 130 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the GAS2-RET fusion nucleic acid molecule of the disclosure encodes a RET fusion polypeptide comprising the amino acid sequence of SEQ ID NO: 131 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the HOOK1-RET fusion nucleic acid molecule of the disclosure encodes a RET fusion polypeptide comprising the amino acid sequence of SEQ ID NO: 132 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the LMNA-RET fusion nucleic acid molecule of the disclosure encodes a RET fusion polypeptide comprising the amino acid sequence of SEQ ID NO: 137 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the LMNA-RET fusion nucleic acid molecule of the disclosure encodes a RET fusion polypeptide comprising the amino acid sequence of SEQ ID NO: 138 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the MLPH-RET fusion nucleic acid molecule of the disclosure encodes a RET fusion polypeptide comprising the amino acid sequence of SEQ ID NO: 139 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the MYH9-RET fusion nucleic acid molecule of the disclosure encodes a RET fusion polypeptide comprising the amino acid sequence of SEQ ID NO: 140 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the PCDH15-RET fusion nucleic acid molecule of the disclosure encodes a RET fusion polypeptide comprising the amino acid sequence of SEQ ID NO: 141 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the PIBF1-RET fusion nucleic acid molecule of the disclosure encodes a RET fusion polypeptide comprising the amino acid sequence of SEQ ID NO: 142 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the PIBF1-RET fusion nucleic acid molecule of the disclosure encodes a RET fusion polypeptide comprising the amino acid sequence of SEQ ID NO: 143 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the SLC12A2-RET fusion nucleic acid molecule of the disclosure encodes a RET fusion polypeptide comprising the amino acid sequence of SEQ ID NO: 144 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ZNF485-RET fusion nucleic acid molecule of the disclosure encodes a RET fusion polypeptide comprising the amino acid sequence of SEQ ID NO: 145 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ZNF485-RET fusion nucleic acid molecule of the disclosure encodes a RET fusion polypeptide comprising the amino acid sequence of SEQ ID NO: 146 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto.

In some embodiments, the RET fusion nucleic acid molecule of the disclosure is any of the fusion nucleic acid molecules as described in the Examples, herein.

In some embodiments of any of the RET fusion nucleic acid molecules provided herein, the fusion nucleic acid molecule is a genomic nucleic acid molecule (i.e., genomic DNA or fragments thereof), or a transcribed nucleic acid molecule, e.g., an RNA such as mRNA, or a cDNA, or fragments thereof. In some embodiments of any of the RET fusion nucleic acid molecules provided herein, the fusion nucleic acid molecule is an isolated nucleic acid molecule.

### (ii) RET Fusion Polypeptides

In certain aspects, provided herein are RET fusion polypeptides which comprise at least a portion of a RET polypeptide and at least a portion of a polypeptide encoded by another gene. In some embodiments, a RET fusion polypeptide of the disclosure is a fusion polypeptide encoded by any of the RET fusion nucleic acid molecules provided herein (e.g., any of the RET fusion nucleic acid molecules described above and/or in the Examples herein), or a portion thereof.

In some embodiments, provided herein are RET fusion polypeptides that comprise at least a portion of a RET polypeptide and at least a portion of a polypeptide encoded by another gene, e.g., a CPEB3, OPTN, GRM7, AGBL4, PPP2R5A, OXR1, LRMDA, MGEA5, ADCY1, SAMD4A, HSD17B7P2, ACPP, ZNF248, MKX, MYH14, BMS1, GOLGB1, NTRK2, ABI3BP, ARHGAP19, GP2, SH2D3A, VSTM4, RBMS3, LINC00379, ZNF721, ZSWIM6, SGIP1, DCC, CCNY, WDFY4, EEA1, ELMO1, RAD1, NAALADL2, CCBE1, CEP135, RAI14, ADAMTS14, SORBS1, CSGALNACT2, PCM1, KIAA1217, MPRIP, TFG, SPECC1L, REEP3, RRBP1, ETV6, TAF3, RASGEF1A, TNIP2, SATB1, ALOX5, ANKRD26, CLIP1, DLG5, ERC1, FRMD4A, KIAA1468, NCOA4, PARD3, PRKAR1A, PRKG1, RUFY2, SNRNP70, SQSTM1, TNIP1, TRIM27, TRIM33, CCDC6, TRIM24, FGFR1OP, GAS2, HOOK1, LMNA, MLPH, MYH9, PCDH15, PIBF1, SLC12A2, or ZNF485 gene, or any gene listed in Tables 1-2, In some embodiments, provided herein arc fusion polypeptides encoded by a GOLGB1-RET, GP2-RET, PPP2R5A-RET, GRM7-RET, MYH14-RET, MGEA5-RET, SAMD4A-RET, ACPP-RET, BMS1-RET, CEP135-RET, EEA1-RET, CSGALNACT2-RET, KIAA1217-RET, SORBS1-RET, MPRIP-RET, TFG-RET, SPECC1L-RET, REEP3-RET, RRBP1-RET, ETV6-RET, TAF3-RET, PCM1-RET, TNIP2-RET, SATB1-RET, RET-ADCY1, RET-ZNF248, RET-AGBL4, RET-LRMDA, RET-ARHGAP19, RET-CPEB3, RET-DCC, RET-ELMO1, RET-WDFY4, MKX-RET, RBMS3-RET, SGIP1-RET, ZSWIM6-RET, ALOX5-RET, RET-NTRK2, RET-OXR1, RET-CCBE1, RET-NAALADL2, RAI14-RET, ABI3BP-RET, LINC00379-RET, OPTN-RET, SH2D3A-RET, ZNF721-RET, ADAMTS14-RET, CSGALNACT2-RET, CPEB3-RET, RET-CCNY, RET-RASGEF1A, RET-HSD17B7P2, RET-RAD1, RET-VSTM4, ANKRD26-RET, CLIP1-RET, DLG5-RET, ERC1-RET, FRMD4A-RET, KIAA1468-RET, NCOA4-RET, PARD3-RET, PRKAR1A-RET, PRKG1-RET, RUFY2-RET, SNRNP70-RET, SQSTM1-RET, TNIP1-RET, TRIM27-RET, TRIM33-RET, CCDC6-RET, TRIM24-RET, FGFR1OP-RET, GAS2-RET, HOOK1-RET, LMNA-RET, MLPH-RET, MYH9-RET, PCDH15-RET, PIBF1-RET, SLC12A2-RET, or ZNF485-RET fusion nucleic acid molecule of the disclosure. In some embodiments, provided herein are RET fusion polypeptides encoded by any of the RET fusion nucleic acid molecules as described above, in the Examples herein, and/or in Tables 1-10 herein. In some embodiments, the RET fusion polypeptide comprises a RET kinase domain, or a fragment of a RET kinase domain having RET kinase activity. In some embodiments, the RET fusion polypeptide has RET kinase activity. In some embodiments, the kinase activity is constitutive. In some embodiments, the RET fusion polypeptide is oncogenic. In some embodiments, the RET fusion polypeptide promotes cancer cell survival, angiogenesis, cancer cell proliferation, and any combination thereof. In some embodiments, the RET fusion polypeptide is capable of dimerizing with a RET polypeptide or with another RET fusion polypeptide.

In some embodiments, a RET fusion polypeptide of the disclosure comprises an amino acid sequence encoded by a RET fusion nucleic acid molecule comprising at least a portion of a RET gene and at least a portion of a CPEB3, OPTN, GRM7, AGBL4, PPP2R5A, OXR1, LRMDA, MGEA5, ADCY1, SAMD4A, HSD17B7P2, ACPP, ZNF248, MKX, MYH14, BMS1, GOLGB1, NTRK2, ABI3BP, ARHGAP19, GP2, SH2D3A, VSTM4, RBMS3, LINC00379, ZNF721, ZSWIM6, SGIP1, DCC, CCNY, WDFY4, EEA1, ELMO1, RAD1, NAALADL2, CCBE1, CEP135, RAI14, ADAMTS14, SORBS1, CSGALNACT2, PCM1, KIAA1217, MPRIP, TFG, SPECC1L, REEP3, RRBP1, ETV6, TAF3, RASGEF1A, TNIP2, SATB1, ALOX5, ANKRD26, CLIP1, DLG5, ERC1, FRMD4A, KIAA1468, NCOA4, PARD3, PRKAR1A, PRKG1, RUFY2, SNRNP70, SQSTM1, TNIP1, TRIM27, TRIM33, CCDC6, TRIM24, FGFR1OP, GAS2, HOOK1, LMNA, MLPH, MYH9, PCDH15, PIBF1, SLC12A2, or ZNF485 gene, or of any gene listed in Tables 1-2, wherein the order of the genes in the fusion in 5′ to 3′ direction is as indicated in Table 3. In some embodiments, the RET fusion polypeptide comprises an amino acid sequence encoded by a GOLGB1-RET fusion nucleic acid molecule of the disclosure comprising, in 5′ to 3′ direction, a GOLGB1 gene or a portion thereof fused to a RET gene or a portion thereof. In some embodiments, the RET fusion polypeptide comprises an amino acid sequence encoded by a GP2-RET fusion nucleic acid molecule of the disclosure comprising, in 5′ to 3′ direction, a GP2 gene or a portion thereof fused to a RET gene or a portion thereof. In some embodiments, the RET fusion polypeptide comprises an amino acid sequence encoded by a PPP2R5A-RET fusion nucleic acid molecule of the disclosure comprising, in the 5′ to 3′ direction, a PPP2R5A gene or a portion thereof fused to a RET gene or a portion thereof. In some embodiments, the RET fusion polypeptide comprises an amino acid sequence encoded by a GRM7-RET fusion nucleic acid molecule of the disclosure comprising, in 5′ to 3′ direction, a GRM7 gene or a portion thereof fused to a RET gene or a portion thereof. In some embodiments, the RET fusion polypeptide comprises an amino acid sequence encoded by a MYH14-RET fusion nucleic acid molecule of the disclosure comprising, in 5′ to 3′ direction, a MYH14 gene or a portion thereof fused to a RET gene or a portion thereof. In some embodiments, the RET fusion polypeptide comprises an amino acid sequence encoded by a MGEA5-RET fusion nucleic acid molecule of the disclosure comprising, in 5′ to 3′ direction, a MGEA5 gene or a portion thereof fused to a RET gene or a portion thereof. In some embodiments, the RET fusion polypeptide comprises an amino acid sequence encoded by a SAMD4A-RET fusion nucleic acid molecule of the disclosure comprising, in the 5′ to 3′ direction, a SAMD4A gene or a portion thereof fused to a RET gene or a portion thereof. In some embodiments, the RET fusion polypeptide comprises an amino acid sequence encoded by a ACPP-RET fusion nucleic acid molecule of the disclosure comprising, in 5′ to 3′ direction, a ACPP gene or a portion thereof fused to a RET gene or a portion thereof. In some embodiments, the RET fusion polypeptide comprises an amino acid sequence encoded by a BMS1-RET fusion nucleic acid molecule of the disclosure comprising, in 5′ to 3′ direction, a BMS1 gene or a portion thereof fused to a RET gene or a portion thereof. In some embodiments, the RET fusion polypeptide comprises an amino acid sequence encoded by a CEP135-RET fusion nucleic acid molecule of the disclosure comprising, in 5′ to 3′ direction, a CEP135 gene or a portion thereof fused to a RET gene or a portion thereof. In some embodiments, the RET fusion polypeptide comprises an amino acid sequence encoded by a EEA1-RET fusion nucleic acid molecule of the disclosure comprising, in the 5′ to 3′ direction, a EEA1 gene or a portion thereof fused to a RET gene or a portion thereof. In some embodiments, the RET fusion polypeptide comprises an amino acid sequence encoded by a CSGALNACT2-RET fusion nucleic acid molecule of the disclosure comprising, in 5′ to 3′ direction, a CSGALNACT2 gene or a portion thereof fused to a RET gene or a portion thereof. In some embodiments, the RET fusion polypeptide comprises an amino acid sequence encoded by a KIAA1217-RET fusion nucleic acid molecule of the disclosure comprising, in 5′ to 3′ direction, a KIAA1217 gene or a portion thereof fused to a RET gene or a portion thereof. In some embodiments, the RET fusion polypeptide comprises an amino acid sequence encoded by a SORBS1-RET fusion nucleic acid molecule of the disclosure comprising, in 5′ to 3′ direction, a SORBS1 gene or a portion thereof fused to a RET gene or a portion thereof. In some embodiments, the RET fusion polypeptide comprises an amino acid sequence encoded by a MPRIP-RET fusion nucleic acid molecule of the disclosure comprising, in 5′ to 3′ direction, a MPRIP gene or a portion thereof fused to a RET gene or a portion thereof. In some embodiments, the RET fusion polypeptide comprises an amino acid sequence encoded by a TFG-RET fusion nucleic acid molecule of the disclosure comprising, in 5′ to 3′ direction, a TFG gene or a portion thereof fused to a RET gene or a portion thereof. In some embodiments, the RET fusion polypeptide comprises an amino acid sequence encoded by a SPECC1L-RET fusion nucleic acid molecule of the disclosure comprising, in the 5′ to 3′ direction, a SPECC1L gene or a portion thereof fused to a RET gene or a portion thereof. In some embodiments, the RET fusion polypeptide comprises an amino acid sequence encoded by a REEP3-RET fusion nucleic acid molecule of the disclosure comprising, in 5′ to 3′ direction, a REEP3 gene or a portion thereof fused to a RET gene or a portion thereof. In some embodiments, the RET fusion polypeptide comprises an amino acid sequence encoded by a RRBP1-RET fusion nucleic acid molecule of the disclosure comprising, in 5′ to 3′ direction, a RRBP1 gene or a portion thereof fused to a RET gene or a portion thereof. In some embodiments, the RET fusion polypeptide comprises an amino acid sequence encoded by a ETV6-RET fusion nucleic acid molecule of the disclosure comprising, in 5′ to 3′ direction, a ETV6 gene or a portion thereof fused to a RET gene or a portion thereof. In some embodiments, the RET fusion polypeptide comprises an amino acid sequence encoded by a TAF3-RET fusion nucleic acid molecule of the disclosure comprising, in the 5′ to 3′ direction, a TAF3 gene or a portion thereof fused to a RET gene or a portion thereof. In some embodiments, the RET fusion polypeptide comprises an amino acid sequence encoded by a PCM1-RET fusion nucleic acid molecule of the disclosure comprising, in 5′ to 3′ direction, a PCM1 gene or a portion thereof fused to a RET gene or a portion thereof. In some embodiments, the RET fusion polypeptide comprises an amino acid sequence encoded by a TNIP2-RET fusion nucleic acid molecule of the disclosure comprising, in 5′ to 3′ direction, a TNIP2 gene or a portion thereof fused to a RET gene or a portion thereof. In some embodiments, the RET fusion polypeptide comprises an amino acid sequence encoded by a SATB1-RET fusion nucleic acid molecule of the disclosure comprising, in 5′ to 3′ direction, a SATB1 gene or a portion thereof fused to a RET gene or a portion thereof. In some embodiments, the RET fusion polypeptide comprises an amino acid sequence encoded by a RET-ADCY1 fusion nucleic acid molecule of the disclosure comprising, in the 5′ to 3′ direction, a RET gene or a portion thereof fused to a ADCY1 gene or a portion thereof. In some embodiments, the RET fusion polypeptide comprises an amino acid sequence encoded by a RET-ZNF248 fusion nucleic acid molecule of the disclosure comprising, in 5′ to 3′ direction, a RET gene or a portion thereof fused to a ZNF248 gene or a portion thereof. In some embodiments, the RET fusion polypeptide comprises an amino acid sequence encoded by a RET-AGBL4 fusion nucleic acid molecule of the disclosure comprising, in 5′ to 3′ direction, a RET gene or a portion thereof fused to a AGBL4 gene or a portion thereof. In some embodiments, the RET fusion polypeptide comprises an amino acid sequence encoded by a RET-LRMDA fusion nucleic acid molecule of the disclosure comprising, in 5′ to 3′ direction, a RET gene or a portion thereof fused to a LRMDA gene or a portion thereof. In some embodiments, the RET fusion polypeptide comprises an amino acid sequence encoded by a RET-ARHGAP19 fusion nucleic acid molecule of the disclosure comprising, in 5′ to 3′ direction, a RET gene or a portion thereof fused to a ARHGAP19 gene or a portion thereof. In some embodiments, the RET fusion polypeptide comprises an amino acid sequence encoded by a RET-CPEB3 fusion nucleic acid molecule of the disclosure comprising, in 5′ to 3″ direction, a RET gene or a portion thereof fused to a CPEB3 gene or a portion thereof. In some embodiments, the RET fusion polypeptide comprises an amino acid sequence encoded by a RET-DCC fusion nucleic acid molecule of the disclosure comprising, in 5′ to 3′ direction, a RET gene or a portion thereof fused to a DCC gene or a portion thereof. In some embodiments, the RET fusion polypeptide comprises an amino acid sequence encoded by a RET-ELMO1 fusion nucleic acid molecule of the disclosure comprising, in 5′ to 3′ direction, a RET gene or a portion thereof fused to a ELMO1 gene or a portion thereof. In some embodiments, the RET fusion polypeptide comprises an amino acid sequence encoded by a RET-WDFY4 fusion nucleic acid molecule of the disclosure comprising, in 5′ to 3′ direction, a RET gene or a portion thereof fused to a WDFY4 gene or a portion thereof. In some embodiments, the RET fusion polypeptide comprises an amino acid sequence encoded by a MKX-RET fusion nucleic acid molecule of the disclosure comprising, in 5′ to 3′ direction, a MKX gene or a portion thereof fused to a RET gene or a portion thereof. In some embodiments, the RET fusion polypeptide comprises an amino acid sequence encoded by a RBMS3-RET fusion nucleic acid molecule of the disclosure comprising, in 5′ to 3′ direction, a RBMS3 gene or a portion thereof fused to a RET gene or a portion thereof. In some embodiments, the RET fusion polypeptide comprises an amino acid sequence encoded by a SGIP1-RET fusion nucleic acid molecule of the disclosure comprising, in 5′ to 3′ direction, a SGIP1 gene or a portion thereof fused to a RET gene or a portion thereof. In some embodiments, the RET fusion polypeptide comprises an amino acid sequence encoded by a ZSWIM6-RET fusion nucleic acid molecule of the disclosure comprising, in 5′ to 3′ direction, a ZSWIM6 gene or a portion thereof fused to a RET gene or a portion thereof. In some embodiments, the RET fusion polypeptide comprises an amino acid sequence encoded by a ALOX5-RET fusion nucleic acid molecule of the disclosure comprising, in the 5′ to 3′ direction, a ALOX5 gene or a portion thereof fused to a RET gene or a portion thereof. In some embodiments, the RET fusion polypeptide comprises an amino acid sequence encoded by a RET-NTRK2 fusion nucleic acid molecule of the disclosure comprising, in 5′ to 3′ direction, a RET gene or a portion thereof fused to a NTRK2 gene or a portion thereof. In some embodiments, the RET fusion polypeptide comprises an amino acid sequence encoded by a RET-OXR1 fusion nucleic acid molecule of the disclosure comprising, in 5′ to 3′ direction, a RET gene or a portion thereof fused to a OXR1 gene or a portion thereof. In some embodiments, the RET fusion polypeptide comprises an amino acid sequence encoded by a RET-CCBE1 fusion nucleic acid molecule of the disclosure comprising, in 5′ to 3′ direction, a RET gene or a portion thereof fused to a CCBE1 gene or a portion thereof. In some embodiments, the RET fusion polypeptide comprises an amino acid sequence encoded by a RET-NAALADL2 fusion nucleic acid molecule of the disclosure comprising, in 5′ to 3′ direction, a RET gene or a portion thereof fused to a NAALADL2 gene or a portion thereof. In some embodiments, the RET fusion polypeptide comprises an amino acid sequence encoded by a RAI14-RET fusion nucleic acid molecule of the disclosure comprising, in 5′ to 3′ direction, a RAI14 gene or a portion thereof fused to a RET gene or a portion thereof. In some embodiments, the RET fusion polypeptide comprises an amino acid sequence encoded by a ABI3BP-RET fusion nucleic acid molecule of the disclosure comprising, in 5′ to 3′ direction, a ABI3BP gene or a portion thereof fused to a RET gene or a portion thereof. In some embodiments, the RET fusion polypeptide comprises an amino acid sequence encoded by a LINC00379-RET fusion nucleic acid molecule of the disclosure comprising, in 5′ to 3′ direction, a LINC00379 gene or a portion thereof fused to a RET gene or a portion thereof. In some embodiments, the RET fusion polypeptide comprises an amino acid sequence encoded by a OPTN-RET fusion nucleic acid molecule of the disclosure comprising, in 5′ to 3′ direction, a OPTN gene or a portion thereof fused to a RET gene or a portion thereof. In some embodiments, the RET fusion polypeptide comprises an amino acid sequence encoded by a SH2D3A-RET fusion nucleic acid molecule of the disclosure comprising, in the 5′ to 3′ direction, a SH2D3A gene or a portion thereof fused to a RET gene or a portion thereof. In some embodiments, the RET fusion polypeptide comprises an amino acid sequence encoded by a ZNF721-RET fusion nucleic acid molecule of the disclosure comprising, in the 5′ to 3′ direction, a ZNF721 gene or a portion thereof fused to a RET gene or a portion thereof. In some embodiments, the RET fusion polypeptide comprises an amino acid sequence encoded by a ADAMTS14-RET fusion nucleic acid molecule of the disclosure comprising, in 5′ to 3′ direction, a ADAMTS14 gene or a portion thereof fused to a RET gene or a portion thereof. In some embodiments, the RET fusion polypeptide comprises an amino acid sequence encoded by a CSGALNACT2-RET fusion nucleic acid molecule of the disclosure comprising, in 5′ to 3′ direction, a CSGALNACT2 gene or a portion thereof fused to a RET gene or a portion thereof. In some embodiments, the RET fusion polypeptide comprises an amino acid sequence encoded by a CPEB3-RET fusion nucleic acid molecule of the disclosure comprising, in 5′ to 3′ direction, a CPEB3 gene or a portion thereof fused to a RET gene or a portion thereof. In some embodiments, the RET fusion polypeptide comprises an amino acid sequence encoded by a RET-CCNY fusion nucleic acid molecule of the disclosure comprising, in 5′ to 3′ direction, a RET gene or a portion thereof fused to a CCNY gene or a portion thereof. In some embodiments, the RET fusion polypeptide comprises an amino acid sequence encoded by a RET-RASGEF1A fusion nucleic acid molecule of the disclosure comprising, in 5′ to 3′ direction, a RET gene or a portion thereof fused to a RASGEF1A gene or a portion thereof. In some embodiments, the RET fusion polypeptide comprises an amino acid sequence encoded by a RET-HSD17B7P2 fusion nucleic acid molecule of the disclosure comprising, in the 5″ to 3′ direction, a RET gene or a portion thereof fused to a HSD17B7P2 gene or a portion thereof. In some embodiments, the RET fusion polypeptide comprises an amino acid sequence encoded by a RET-RAD1 fusion nucleic acid molecule of the disclosure comprising, in 5′ to 3′ direction, a RET gene or a portion thereof fused to a RAD1 gene or a portion thereof. In some embodiments, the RET fusion polypeptide comprises an amino acid sequence encoded by a RET-VSTM4 fusion nucleic acid molecule of the disclosure comprising, in 5′ to 3′ direction, a RET gene or a portion thereof fused to a VSTM4 gene or a portion thereof. In some embodiments, the RET fusion polypeptide comprises an amino acid sequence encoded by an ANKRD26-RET fusion nucleic acid molecule of the disclosure comprising, in 5′ to 3′ direction, an ANKRD26 gene or a portion thereof fused to a RET gene or a portion thereof. In some embodiments, the RET fusion polypeptide comprises an amino acid sequence encoded by a CLIP1-RET fusion nucleic acid molecule of the disclosure comprising, in 5′ to 3′ direction, a CLIP1 gene or a portion thereof fused to a RET gene or a portion thereof. In some embodiments, the RET fusion polypeptide comprises an amino acid sequence encoded by a DLG5-RET fusion nucleic acid molecule of the disclosure comprising, in 5′ to 3″ direction, a DLG5 gene or a portion thereof fused to a RET gene or a portion thereof. In some embodiments, the RET fusion polypeptide comprises an amino acid sequence encoded by an ERC1-RET fusion nucleic acid molecule of the disclosure comprising, in 5′ to 3′ direction, an ERC1 gene or a portion thereof fused to a RET gene or a portion thereof. In some embodiments, the RET fusion polypeptide comprises an amino acid sequence encoded by a FRMD4A-RET fusion nucleic acid molecule of the disclosure comprising, in 5′ to 3′ direction, a FRMD4A gene or a portion thereof fused to a RET gene or a portion thereof. In some embodiments, the RET fusion polypeptide comprises an amino acid sequence encoded by a KIAA1468-RET fusion nucleic acid molecule of the disclosure comprising, in 5′ to 3′ direction, a KIAA1468 gene or a portion thereof fused to a RET gene or a portion thereof. In some embodiments, the RET fusion polypeptide comprises an amino acid sequence encoded by a NCOA4-RET fusion nucleic acid molecule of the disclosure comprising, in the 5′ to 3′ direction, a NCOA4 gene or a portion thereof fused to a RET gene or a portion thereof. In some embodiments, the RET fusion polypeptide comprises an amino acid sequence encoded by a PARD3-RET fusion nucleic acid molecule of the disclosure comprising, in 5′ to 3′ direction, a PARD3 gene or a portion thereof fused to a RET gene or a portion thereof. In some embodiments, the RET fusion polypeptide comprises an amino acid sequence encoded by a PRKAR1A-RET fusion nucleic acid molecule of the disclosure comprising, in 5′ to 3′ direction, a PRKAR1A gene or a portion thereof fused to a RET gene or a portion thereof. In some embodiments, the RET fusion polypeptide comprises an amino acid sequence encoded by a PRKG1-RET fusion nucleic acid molecule of the disclosure comprising, in 5′ to 3′ direction, a PRKG1 gene or a portion thereof fused to a RET gene or a portion thereof. In some embodiments, the RET fusion polypeptide comprises an amino acid sequence encoded by a RUFY2-RET fusion nucleic acid molecule of the disclosure comprising, in 5′ to 3′ direction, a RUFY2 gene or a portion thereof fused to a RET gene or a portion thereof. In some embodiments, the RET fusion polypeptide comprises an amino acid sequence encoded by a SNRNP70-RET fusion nucleic acid molecule of the disclosure comprising, in the 5′ to 3′ direction, a SNRNP70 gene or a portion thereof fused to a RET gene or a portion thereof. In some embodiments, the RET fusion polypeptide comprises an amino acid sequence encoded by a SQSTM1-RET fusion nucleic acid molecule of the disclosure comprising, in 5′ to 3′ direction, a SQSTM1 gene or a portion thereof fused to a RET gene or a portion thereof. In some embodiments, the RET fusion polypeptide comprises an amino acid sequence encoded by a TNIP1-RET fusion nucleic acid molecule of the disclosure comprising, in 5′ to 3′ direction, a TNIP1 gene or a portion thereof fused to a RET gene or a portion thereof. In some embodiments, the RET fusion polypeptide comprises an amino acid sequence encoded by a TRIM27-RET fusion nucleic acid molecule of the disclosure comprising, in 5′ to 3′ direction, a TRIM27 gene or a portion thereof fused to a RET gene or a portion thereof. In some embodiments, the RET fusion polypeptide comprises an amino acid sequence encoded by a TRIM33-RET fusion nucleic acid molecule of the disclosure comprising, in 5′ to 3′ direction, a TRIM33 gene or a portion thereof fused to a RET gene or a portion thereof. In some embodiments, the RET fusion polypeptide comprises an amino acid sequence encoded by a CCDC6-RET fusion nucleic acid molecule of the disclosure comprising, in the 5′ to 3′ direction, a CCDC6 gene or a portion thereof fused to a RET gene or a portion thereof. In some embodiments, the RET fusion polypeptide comprises an amino acid sequence encoded by a TRIM24-RET fusion nucleic acid molecule of the disclosure comprising, in 5′ to 3′ direction, a TRIM24 gene or a portion thereof fused to a RET gene or a portion thereof. In some embodiments, the RET fusion polypeptide comprises an amino acid sequence encoded by a FGFR1OP-RET fusion nucleic acid molecule of the disclosure comprising, in 5′ to 3′ direction, a FGFR1OP gene or a portion thereof fused to a RET gene or a portion thereof. In some embodiments, the RET fusion polypeptide comprises an amino acid sequence encoded by a GAS2-RET fusion nucleic acid molecule of the disclosure comprising, in 5′ to 3′ direction, a GAS2 gene or a portion thereof fused to a RET gene or a portion thereof. In some embodiments, the RET fusion polypeptide comprises an amino acid sequence encoded by a HOOK1-RET fusion nucleic acid molecule of the disclosure comprising, in 5′ to 3′ direction, a HOOK1 gene or a portion thereof fused to a RET gene or a portion thereof. In some embodiments, the RET fusion polypeptide comprises an amino acid sequence encoded by a LMNA-RET fusion nucleic acid molecule of the disclosure comprising, in 5′ to 3′ direction, a LMNA gene or a portion thereof fused to a RET gene or a portion thereof. In some embodiments, the RET fusion polypeptide comprises an amino acid sequence encoded by a MLPH-RET fusion nucleic acid molecule of the disclosure comprising, in 5′ to 3′ direction, a MLPH gene or a portion thereof fused to a RET gene or a portion thereof. In some embodiments, the RET fusion polypeptide comprises an amino acid sequence encoded by a MYH9-RET fusion nucleic acid molecule of the disclosure comprising, in 5′ to 3′ direction, a MYH9 gene or a portion thereof fused to a RET gene or a portion thereof. In some embodiments, the RET fusion polypeptide comprises an amino acid sequence encoded by a PCDH15-RET fusion nucleic acid molecule of the disclosure comprising, in 5′ to 3′ direction, a PCDH15 gene or a portion thereof fused to a RET gene or a portion thereof. In some embodiments, the RET fusion polypeptide comprises an amino acid sequence encoded by a PIBF1-RET fusion nucleic acid molecule of the disclosure comprising, in the 5′ to 3′ direction, a PIBF1 gene or a portion thereof fused to a RET gene or a portion thereof. In some embodiments, the RET fusion polypeptide comprises an amino acid sequence encoded by a SLC12A2-RET fusion nucleic acid molecule of the disclosure comprising, in 5′ to 3′ direction, a SLC12A2 gene or a portion thereof fused to a RET gene or a portion thereof. In some embodiments, the RET fusion polypeptide comprises an amino acid sequence encoded by a ZNF485-RET fusion nucleic acid molecule of the disclosure comprising, in 5′ to 3′ direction, a ZNF485 gene or a portion thereof fused to a RET gene or a portion thereof.

In some embodiments, a RET fusion polypeptide of the disclosure comprises an amino acid sequence encoded by a RET fusion nucleic acid molecule of the disclosure that comprises or results from a 5′ breakpoint and/or a 3′ breakpoint within the corresponding exons or introns as indicated in Table 4. In some embodiments, the RET fusion polypeptide comprises an amino acid sequence encoded by a GOLGB1-RET fusion nucleic acid molecule of the disclosure that comprises or results from a 5′ breakpoint within GOLGB1 intron 10 and/or a 3′ breakpoint within RET intron 11. In some embodiments, the RET fusion polypeptide comprises an amino acid sequence encoded by a GP2-RET fusion nucleic acid molecule of the disclosure that comprises or results from a 5′ breakpoint within GP2 intron 4 and/or a 3′ breakpoint within RET intron 10. In some embodiments, the RET fusion polypeptide comprises an amino acid sequence encoded by a PPP2R5A-RET fusion nucleic acid molecule of the disclosure that comprises or results from a 5′ breakpoint within PPP2R5A intron 1 and/or a 3′ breakpoint within RET intron 19. In some embodiments, the RET fusion polypeptide comprises an amino acid sequence encoded by a GRM7-RET fusion nucleic acid molecule of the disclosure that comprises or results from a 5′ breakpoint within GRM7 intron 9 and/or a 3′ breakpoint within RET intron 2. In some embodiments, the RET fusion polypeptide comprises an amino acid sequence encoded by a MYH14-RET fusion nucleic acid molecule of the disclosure that comprises or results from a 5′ breakpoint within MYH14 intron 37 and/or a 3′ breakpoint within RET intron 11. In some embodiments, the RET fusion polypeptide comprises an amino acid sequence encoded by a MGEA5-RET fusion nucleic acid molecule of the disclosure that comprises or results from a 5′ breakpoint within MGEA5 intron 12 and/or a 3′ breakpoint within RET intron 11. In some embodiments, the RET fusion polypeptide comprises an amino acid sequence encoded by a SAMD4A-RET fusion nucleic acid molecule of the disclosure that comprises or results from a 5′ breakpoint within SAMD4A intron 2 and/or a 3′ breakpoint within RET intron 10. In some embodiments, the RET fusion polypeptide comprises an amino acid sequence encoded by a ACPP-RET fusion nucleic acid molecule of the disclosure that comprises or results from a 5′ breakpoint within ACPP intron 10 and/or a 3′ breakpoint within RET intron 10. In some embodiments, the RET fusion polypeptide comprises an amino acid sequence encoded by a BMS1-RET fusion nucleic acid molecule of the disclosure that comprises or results from a 5′ breakpoint within BMS1 intron 13 and/or a 3′ breakpoint within RET intron 9. In some embodiments, the RET fusion polypeptide comprises an amino acid sequence encoded by a CEP135-RET fusion nucleic acid molecule of the disclosure that comprises or results from a 5′ breakpoint within CEP135 intron 11 and/or a 3′ breakpoint within RET intron 11. In some embodiments, the RET fusion polypeptide comprises an amino acid sequence encoded by a EEA1-RET fusion nucleic acid molecule of the disclosure that comprises or results from a 5′ breakpoint within EEA1 intron 13 and/or a 3′ breakpoint within RET intron 11. In some embodiments, the RET fusion polypeptide comprises an amino acid sequence encoded by a CSGALNACT2-RET fusion nucleic acid molecule of the disclosure that comprises or results from a 5′ breakpoint within CSGALNACT2 intron 6 and/or a 3′ breakpoint within RET intron 11. In some embodiments, the RET fusion polypeptide comprises an amino acid sequence encoded by a KIAA1217-RET fusion nucleic acid molecule of the disclosure that comprises or results from a 5′ breakpoint within KIAA1217 intron 11 and/or a 3′ breakpoint within RET intron 7. In some embodiments, the RET fusion polypeptide comprises an amino acid sequence encoded by a SORBS1-RET fusion nucleic acid molecule of the disclosure that comprises or results from a 5′ breakpoint within SORBS1 intron 20 and/or a 3′ breakpoint within RET intron 6. In some embodiments, the RET fusion polypeptide comprises an amino acid sequence encoded by a SORBS1-RET fusion nucleic acid molecule of the disclosure that comprises or results from a 5′ breakpoint within SORBS1 intron 22 and/or a 3′ breakpoint within RET intron 10. In some embodiments, the RET fusion polypeptide comprises an amino acid sequence encoded by a MPRIP-RET fusion nucleic acid molecule of the disclosure that comprises or results from a 5′ breakpoint within MPRIP intron 22 and/or a 3′ breakpoint within RET intron 11. In some embodiments, the RET fusion polypeptide comprises an amino acid sequence encoded by a TFG-RET fusion nucleic acid molecule of the disclosure that comprises or results from a 5′ breakpoint within TFG intron 5 and/or a 3′ breakpoint within RET intron 10. In some embodiments, the RET fusion polypeptide comprises an amino acid sequence encoded by a SPECC1L-RET fusion nucleic acid molecule of the disclosure that comprises or results from a 5′ breakpoint within SPECC1L intron 10 and/or a 3′ breakpoint within RET intron 11. In some embodiments, the RET fusion polypeptide comprises an amino acid sequence encoded by a REEP3-RET fusion nucleic acid molecule of the disclosure that comprises or results from a 5′ breakpoint within REEP3 intron 5 and/or a 3′ breakpoint within RET intron 11. In some embodiments, the RET fusion polypeptide comprises an amino acid sequence encoded by a RRBP1-RET fusion nucleic acid molecule of the disclosure that comprises or results from a 5′ breakpoint within RRBP1 intron 22 and/or a 3′ breakpoint within RET intron 11. In some embodiments, the RET fusion polypeptide comprises an amino acid sequence encoded by a ETV6-RET fusion nucleic acid molecule of the disclosure that comprises or results from a 5′ breakpoint within ETV6 intron 6 and/or a 3′ breakpoint within RET intron 11. In some embodiments, the RET fusion polypeptide comprises an amino acid sequence encoded by a TAF3-RET fusion nucleic acid molecule of the disclosure that comprises or results from a 5′ breakpoint within TAF3 intron 3 and/or a 3′ breakpoint within RET intron 11. In some embodiments, the RET fusion polypeptide comprises an amino acid sequence encoded by a PCM1-RET fusion nucleic acid molecule of the disclosure that comprises or results from a 5′ breakpoint within PCM1 intron 29 and/or a 3′ breakpoint within RET intron 11. In some embodiments, the RET fusion polypeptide comprises an amino acid sequence encoded by a TNIP2-RET fusion nucleic acid molecule of the disclosure that comprises or results from a 5′ breakpoint within TNIP2 intron 5 and/or a 3′ breakpoint within RET intron 11. In some embodiments, the RET fusion polypeptide comprises an amino acid sequence encoded by a SATB1-RET fusion nucleic acid molecule of the disclosure that comprises or results from a 5′ breakpoint within SATB1 intron 7 and/or a 3′ breakpoint within RET intron 11. In some embodiments, the RET fusion polypeptide comprises an amino acid sequence encoded by a RET-ADCY1 fusion nucleic acid molecule of the disclosure that comprises or results from a 5′ breakpoint within RET intron 11 and/or a 3′ breakpoint within ADCY1 intron 11. In some embodiments, the RET fusion polypeptide comprises an amino acid sequence encoded by a RET-ZNF248 fusion nucleic acid molecule of the disclosure that comprises or results from a 5′ breakpoint within RET intron 11 and/or a 3′ breakpoint within ZNF248 intron 3. In some embodiments, the RET fusion polypeptide comprises an amino acid sequence encoded by a RET-AGBL4 fusion nucleic acid molecule of the disclosure that comprises or results from a 5′ breakpoint within RET intron 12 and/or a 3′ breakpoint within AGBL4 intron 2. In some embodiments, the RET fusion polypeptide comprises an amino acid sequence encoded by a RET-LRMDA fusion nucleic acid molecule of the disclosure that comprises or results from a 5′ breakpoint within RET intron 10 and/or a 3′ breakpoint within LRMDA intron 5. In some embodiments, the RET fusion polypeptide comprises an amino acid sequence encoded by a RET-ARHGAP19 fusion nucleic acid molecule of the disclosure that comprises or results from a 5′ breakpoint within RET intron 11 and/or a 3′ breakpoint within ARHGAP19 intron 6. In some embodiments, the RET fusion polypeptide comprises an amino acid sequence encoded by a RET-CPEB3 fusion nucleic acid molecule of the disclosure that comprises or results from a 5′ breakpoint within RET intron 11 and/or a 3′ breakpoint within CPEB3 intron 7. In some embodiments, the RET fusion polypeptide comprises an amino acid sequence encoded by a RET-DCC fusion nucleic acid molecule of the disclosure that comprises or results from a 5′ breakpoint within RET intron 9 and/or a 3′ breakpoint within DCC intron 7. In some embodiments, the RET fusion polypeptide comprises an amino acid sequence encoded by a RET-ELMO1 fusion nucleic acid molecule of the disclosure that comprises or results from a 5′ breakpoint within RET intron 11 and/or a 3′ breakpoint within ELMO1 intron 13. In some embodiments, the RET fusion polypeptide comprises an amino acid sequence encoded by a RET-WDFY4 fusion nucleic acid molecule of the disclosure that comprises or results from a 5′ breakpoint within RET intron 15 and/or a 3′ breakpoint within WDFY4 intron 39. In some embodiments, the RET fusion polypeptide comprises an amino acid sequence encoded by a MKX-RET fusion nucleic acid molecule of the disclosure that comprises or results from a 5′ breakpoint within MKX intron 5 and/or a 3′ breakpoint within RET intron 11. In some embodiments, the RET fusion polypeptide comprises an amino acid sequence encoded by a RBMS3-RET fusion nucleic acid molecule of the disclosure that comprises or results from a 5′ breakpoint within RBMS3 intron 6 and/or a 3″ breakpoint within RET intron 11. In some embodiments, the RET fusion polypeptide comprises an amino acid sequence encoded by a SGIP1-RET fusion nucleic acid molecule of the disclosure that comprises or results from a 5′ breakpoint within SGIP1 intron 1 and/or a 3′ breakpoint within RET intron 11. In some embodiments, the RET fusion polypeptide comprises an amino acid sequence encoded by a ZSWIM6-RET fusion nucleic acid molecule of the disclosure that comprises or results from a 5′ breakpoint within ZSWIM6 intron 1 and/or a 3′ breakpoint within RET intron 11. In some embodiments, the RET fusion polypeptide comprises an amino acid sequence encoded by a ALOX5-RET fusion nucleic acid molecule of the disclosure that comprises or results from a 5′ breakpoint within ALOX5 intron 2 and/or a 3′ breakpoint within RET intron 11. In some embodiments, the RET fusion polypeptide comprises an amino acid sequence encoded by a RET-NTRK2 fusion nucleic acid molecule of the disclosure that comprises or results from a 5′ breakpoint within RET intron 10 and/or a 3′ breakpoint within NTRK2 intron 19. In some embodiments, the RET fusion polypeptide comprises an amino acid sequence encoded by a RET-OXR1 fusion nucleic acid molecule of the disclosure that comprises or results from a 5′ breakpoint within RET intron 11 and/or a 3′ breakpoint within OXR1 intron 3. In some embodiments, the RET fusion polypeptide comprises an amino acid sequence encoded by a RET-CCBE1 fusion nucleic acid molecule of the disclosure that comprises or results from a 5′ breakpoint within RET intron 8 and/or a 3′ breakpoint within CCBE1 intron 2. In some embodiments, the RET fusion polypeptide comprises an amino acid sequence encoded by a RET-NAALADL2 fusion nucleic acid molecule of the disclosure that comprises or results from a 5′ breakpoint within RET intron 11 and/or a 3′ breakpoint within NAALADL2 intron 12. In some embodiments, the RET fusion polypeptide comprises an amino acid sequence encoded by a RAI14-RET fusion nucleic acid molecule of the disclosure that comprises or results from a 5′ breakpoint within RAI14 intron 15 and/or a 3′ breakpoint within RET exon 11. In some embodiments, the RET fusion polypeptide comprises an amino acid sequence encoded by a ABI3BP-RET fusion nucleic acid molecule of the disclosure that comprises or results from a 5′ breakpoint within ABI3BP exon 35 and/or a 3′ breakpoint within RET intron 11. In some embodiments, the RET fusion polypeptide comprises an amino acid sequence encoded by a LINC00379-RET fusion nucleic acid molecule of the disclosure that comprises or results from a 5′ breakpoint within LINC00379 intron 2 and/or a 3″ breakpoint within RET intron 11. In some embodiments, the RET fusion polypeptide comprises an amino acid sequence encoded by a OPTN-RET fusion nucleic acid molecule of the disclosure that comprises or results from a 5′ breakpoint within OPTN intron 1 and/or a 3′ breakpoint within RET intron 10. In some embodiments, the RET fusion polypeptide comprises an amino acid sequence encoded by a SH2D3A-RET fusion nucleic acid molecule of the disclosure that comprises or results from a 5′ breakpoint within SH2D3A intron 1 and/or a 3′ breakpoint within RET intron 11. In some embodiments, the RET fusion polypeptide comprises an amino acid sequence encoded by a ZNF721-RET fusion nucleic acid molecule of the disclosure that comprises or results from a 5′ breakpoint within ZNF721 intron I and/or a 3′ breakpoint within RET intron 11. In some embodiments, the RET fusion polypeptide comprises an amino acid sequence encoded by a ADAMTS14-RET fusion nucleic acid molecule of the disclosure that comprises or results from a 5′ breakpoint within ADAMTS14 intron 4 and/or a 3′ breakpoint within RET intron 11. In some embodiments, the RET fusion polypeptide comprises an amino acid sequence encoded by a CSGALNACT2-RET fusion nucleic acid molecule of the disclosure that comprises or results from a 5′ breakpoint within CSGALNACT2 intron 1 and/or a 3′ breakpoint within RET intron 11. In some embodiments, the RET fusion polypeptide comprises an amino acid sequence encoded by a CPEB3-RET fusion nucleic acid molecule of the disclosure that comprises or results from a 5′ breakpoint within CPEB3 exon 10 and/or a 3′ breakpoint within RET intron 11. In some embodiments, the RET fusion polypeptide comprises an amino acid sequence encoded by a RET-CCNY fusion nucleic acid molecule of the disclosure that comprises or results from a 5′ breakpoint within RET intron 11 and/or a 3′ breakpoint within CCNY intron 3. In some embodiments, the RET fusion polypeptide comprises an amino acid sequence encoded by a RET-RASGEF1A fusion nucleic acid molecule of the disclosure that comprises or results from a 5′ breakpoint within RET intron 5 and/or a 3′ breakpoint within RASGEF1A intron 1. In some embodiments, the RET fusion polypeptide comprises an amino acid sequence encoded by a RET-HSD17B7P2 fusion nucleic acid molecule of the disclosure that comprises or results from a 5′ breakpoint within RET exon 11 and/or a 3′ breakpoint within HSD17B7P2 exon 8. In some embodiments, the RET fusion polypeptide comprises an amino acid sequence encoded by a RET-RAD1 fusion nucleic acid molecule of the disclosure that comprises or results from a 5′ breakpoint within RET exon 11 and/or a 3′ breakpoint within RAD1 exon 6. In some embodiments, the RET fusion polypeptide comprises an amino acid sequence encoded by a RET-VSTM4 fusion nucleic acid molecule of the disclosure that comprises or results from a 5′ breakpoint within RET intron 10 and/or a 3′ breakpoint within VSTM4 exon 8. In some embodiments, the RET fusion polypeptide comprises an amino acid sequence encoded by an ANKRD26-RET fusion nucleic acid molecule of the disclosure that comprises or results from a 5′ breakpoint within ANKRD26 intron 29 and/or a 3′ breakpoint within RET intron 11. In some embodiments, the RET fusion polypeptide comprises an amino acid sequence encoded by a CLIP1-RET fusion nucleic acid molecule of the disclosure that comprises or results from a 5′ breakpoint within CLIP1 intron 16 and/or a 3′ breakpoint within RET intron 11. In some embodiments, the RET fusion polypeptide comprises an amino acid sequence encoded by a DLG5-RET fusion nucleic acid molecule of the disclosure that comprises or results from a 5′ breakpoint within DLG5 intron 14 and/or a 3′ breakpoint within RET intron 11. In some embodiments, the RET fusion polypeptide comprises an amino acid sequence encoded by a DLG5-RET fusion nucleic acid molecule of the disclosure that comprises or results from a 5′ breakpoint within DLG5 intron 27 and/or a 3′ breakpoint within RET intron 7. In some embodiments, the RET fusion polypeptide comprises an amino acid sequence encoded by an ERC1-RET fusion nucleic acid molecule of the disclosure that comprises or results from a 5′ breakpoint within ERC1 intron 12 and/or a 3′ breakpoint within RET intron 11. In some embodiments, the RET fusion polypeptide comprises an amino acid sequence encoded by an ERC1-RET fusion nucleic acid molecule of the disclosure that comprises or results from a 5′ breakpoint within ERC1 intron 8 and/or a 3′ breakpoint within RET intron 11. In some embodiments, the RET fusion polypeptide comprises an amino acid sequence encoded by an ERC1-RET fusion nucleic acid molecule of the disclosure that comprises or results from a 5′ breakpoint within ERC1 intron 12 and/or a 3′ breakpoint within RET intron 10. In some embodiments, the RET fusion polypeptide comprises an amino acid sequence encoded by a FRMD4A-RET fusion nucleic acid molecule of the disclosure that comprises or results from a 5′ breakpoint within FRMD4A intron 12 and/or a 3′ breakpoint within RET intron 11. In some embodiments, the RET fusion polypeptide comprises an amino acid sequence encoded by a KIAA1468-RET fusion nucleic acid molecule of the disclosure that comprises or results from a 5′ breakpoint within KIAA1468 intron 10 and/or a 3′ breakpoint within RET intron 11. In some embodiments, the RET fusion polypeptide comprises an amino acid sequence encoded by a NCOA4-RET fusion nucleic acid molecule of the disclosure that comprises or results from a 5′ breakpoint within NCOA4 intron 8 and/or a 3′ breakpoint within RET intron 11. In some embodiments, the RET fusion polypeptide comprises an amino acid sequence encoded by a PARD3-RET fusion nucleic acid molecule of the disclosure that comprises or results from a 5′ breakpoint within PARD3 intron 2 and/or a 3′ breakpoint within RET intron 7. In some embodiments, the RET fusion polypeptide comprises an amino acid sequence encoded by a PARD3-RET fusion nucleic acid molecule of the disclosure that comprises or results from a 5′ breakpoint within PARD3 intron 4 and/or a 3′ breakpoint within RET intron 11. In some embodiments, the RET fusion polypeptide comprises an amino acid sequence encoded by a PRKAR1A-RET fusion nucleic acid molecule of the disclosure that comprises or results from a 5′ breakpoint within PRKAR1A intron 7 and/or a 3′ breakpoint within RET intron 11. In some embodiments, the RET fusion polypeptide comprises an amino acid sequence encoded by a PRKG1-RET fusion nucleic acid molecule of the disclosure that comprises or results from a 5′ breakpoint within PRKG1 intron 7 and/or a 3′ breakpoint within RET intron 3. In some embodiments, the RET fusion polypeptide comprises an amino acid sequence encoded by a PRKG1-RET fusion nucleic acid molecule of the disclosure that comprises or results from a 5′ breakpoint within PRKG1 intron 5 and/or a 3′ breakpoint within RET intron 17. In some embodiments, the RET fusion polypeptide comprises an amino acid sequence encoded by a RUFY2-RET fusion nucleic acid molecule of the disclosure that comprises or results from a 5′ breakpoint within RUFY2 intron 9 and/or a 3′ breakpoint within RET intron 11. In some embodiments, the RET fusion polypeptide comprises an amino acid sequence encoded by a SNRNP70-RET fusion nucleic acid molecule of the disclosure that comprises or results from a 5′ breakpoint within SNRNP70 intron 6 and/or a 3′ breakpoint within RET intron 11. In some embodiments, the RET fusion polypeptide comprises an amino acid sequence encoded by a SQSTM1-RET fusion nucleic acid molecule of the disclosure that comprises or results from a 5′ breakpoint within SQSTM1 intron 5 and/or a 3′ breakpoint within RET intron 10. In some embodiments, the RET fusion polypeptide comprises an amino acid sequence encoded by a TNIP1-RET fusion nucleic acid molecule of the disclosure that comprises or results from a 5′ breakpoint within TNIP1 intron 13 and/or a 3′ breakpoint within RET intron 11. In some embodiments, the RET fusion polypeptide comprises an amino acid sequence encoded by a TNIP1-RET fusion nucleic acid molecule of the disclosure that comprises or results from a 5′ breakpoint within TNIP1 intron 15 and/or a 3′ breakpoint within RET intron 11. In some embodiments, the RET fusion polypeptide comprises an amino acid sequence encoded by a TNIP1-RET fusion nucleic acid molecule of the disclosure that comprises or results from a 5′ breakpoint within TNIP1 intron 12 and/or a 3′ breakpoint within RET intron 11. In some embodiments, the RET fusion polypeptide comprises an amino acid sequence encoded by a TRIM27-RET fusion nucleic acid molecule of the disclosure that comprises or results from a 5′ breakpoint within TRIM27 intron 3 and/or a 3′ breakpoint within RET intron 11. In some embodiments, the RET fusion polypeptide comprises an amino acid sequence encoded by a TRIM33-RET fusion nucleic acid molecule of the disclosure that comprises or results from a 5′ breakpoint within TRIM33 intron 10 and/or a 3′ breakpoint within RET intron 11. In some embodiments, the RET fusion polypeptide comprises an amino acid sequence encoded by a CCDC6-RET fusion nucleic acid molecule of the disclosure that comprises or results from a 5′ breakpoint within CCDC6 intron 3 and/or a 3′ breakpoint within RET intron 10. In some embodiments, the RET fusion polypeptide comprises an amino acid sequence encoded by a TRIM24-RET fusion nucleic acid molecule of the disclosure that comprises or results from a 5′ breakpoint within TRIM24 intron 17 and/or a 3′ breakpoint within RET intron 11. In some embodiments, the RET fusion polypeptide comprises an amino acid sequence encoded by a FGFR1OP-RET fusion nucleic acid molecule of the disclosure that comprises or results from a 5′ breakpoint within FGFR1OP intron 6 and/or a 3′ breakpoint within RET intron 11. In some embodiments, the RET fusion polypeptide comprises an amino acid sequence encoded by a GAS2-RET fusion nucleic acid molecule of the disclosure that comprises or results from a 5′ breakpoint within GAS2 intron 6 and/or a 3′ breakpoint within RET intron 3. In some embodiments, the RET fusion polypeptide comprises an amino acid sequence encoded by a HOOK1-RET fusion nucleic acid molecule of the disclosure that comprises or results from a 5′ breakpoint within HOOK1 intron 20 and/or a 3′ breakpoint within RET intron 11. In some embodiments, the RET fusion polypeptide comprises an amino acid sequence encoded by a LMNA-RET fusion nucleic acid molecule of the disclosure that comprises or results from a 5′ breakpoint within LMNA intron 2 and/or a 3′ breakpoint within RET intron 11. In some embodiments, the RET fusion polypeptide comprises an amino acid sequence encoded by a MLPH-RET fusion nucleic acid molecule of the disclosure that comprises or results from a 5′ breakpoint within MLPH intron 9 and/or a 3′ breakpoint within RET intron 11. In some embodiments, the RET fusion polypeptide comprises an amino acid sequence encoded by a MYH9-RET fusion nucleic acid molecule of the disclosure that comprises or results from a 5′ breakpoint within MYH9 intron 34 and/or a 3′ breakpoint within RET intron 11. In some embodiments, the RET fusion polypeptide comprises an amino acid sequence encoded by a PCDH15-RET fusion nucleic acid molecule of the disclosure that comprises or results from a 5′ breakpoint within PCDH15 intron 1 and/or a 3′ breakpoint within RET intron 4. In some embodiments, the RET fusion polypeptide comprises an amino acid sequence encoded by a PIBF1-RET fusion nucleic acid molecule of the disclosure that comprises or results from a 5′ breakpoint within PIBF1 intron 16 and/or a 3′ breakpoint within RET intron 11. In some embodiments, the RET fusion polypeptide comprises an amino acid sequence encoded by a SLC12A2-RET fusion nucleic acid molecule of the disclosure that comprises or results from a 5′ breakpoint within SLC12A2 intron 16 and/or a 3′ breakpoint within RET intron 11. In some embodiments, the RET fusion polypeptide comprises an amino acid sequence encoded by a ZNF485-RET fusion nucleic acid molecule of the disclosure that comprises or results from a 5′ breakpoint within ZNF485 intron 4 and/or a 3′ breakpoint within RET intron 11. In some embodiments, the RET fusion polypeptide comprises an amino acid sequence encoded by a ZNF485-RET fusion nucleic acid molecule of the disclosure that comprises or results from a 5′ breakpoint within ZNF485 intron 4 and/or a 3′ breakpoint within RET intron 9.

In some embodiments, a RET fusion polypeptide of the disclosure comprises an amino acid sequence encoded by a RET fusion nucleic acid molecule of the disclosure that comprises or results from a 5′ breakpoint and/or a 3′ breakpoint within the corresponding chromosomal coordinates as indicated in Table 5. In some embodiments, the RET fusion polypeptide comprises an amino acid sequence encoded by a GOLGB1-RET fusion nucleic acid molecule of the disclosure that comprises or results from a breakpoint within chromosomal coordinates chr3: 121428577-121428818 and/or chr10: 43611900-43612184. In some embodiments, the RET fusion polypeptide comprises an amino acid sequence encoded by a GP2-RET fusion nucleic acid molecule of the disclosure that comprises or results from a breakpoint within chromosomal coordinates chr16: 20331978-20332315 and/or chr10: 43609279-43609727. In some embodiments, the RET fusion polypeptide comprises an amino acid sequence encoded by a PPP2RSA-RET fusion nucleic acid molecule of the disclosure that comprises or results from a breakpoint within chromosomal coordinates chr1: 212474140-212474319 and/or chr10: 43623512-43623678. In some embodiments, the RET fusion polypeptide comprises an amino acid sequence encoded by a GRM7-RET fusion nucleic acid molecule of the disclosure that comprises or results from a breakpoint within chromosomal coordinates chr3: 7728019-7728192 and/or chr10: 43595834-43596313. In some embodiments, the RET fusion polypeptide comprises an amino acid sequence encoded by a MYH14-RET fusion nucleic acid molecule of the disclosure that comprises or results from a breakpoint within chromosomal coordinates chr19: 50801162-50801465 and/or chr10: 43610285-43610748. In some embodiments, the RET fusion polypeptide comprises an amino acid sequence encoded by a MGEA5-RET fusion nucleic acid molecule of the disclosure that comprises or results from a breakpoint within chromosomal coordinates chr10: 103551711-103551879 and/or chr10: 43610920-43611146. In some embodiments, the RET fusion polypeptide comprises an amino acid sequence encoded by a SAMD4A-RET fusion nucleic acid molecule of the disclosure that comprises or results from a breakpoint within chromosomal coordinates chr14: 55123072-55123234 and/or chr10: 43609336-43609516. In some embodiments, the RET fusion polypeptide comprises an amino acid sequence encoded by a ACPP-RET fusion nucleic acid molecule of the disclosure that comprises or results from a breakpoint within chromosomal coordinates chr3: 132078976-132079163 and/or chr10: 43609901-43610079. In some embodiments, the RET fusion polypeptide comprises an amino acid sequence encoded by a BMS1-RET fusion nucleic acid molecule of the disclosure that comprises or results from a breakpoint within chromosomal coordinates chr10: 43307628-43307807 and/or chr10: 43608772-43609039. In some embodiments, the RET fusion polypeptide comprises an amino acid sequence encoded by a CEP135-RET fusion nucleic acid molecule of the disclosure that comprises or results from a breakpoint within chromosomal coordinates chr4: 56844220-56844527 and/or chr10: 43611070-43611610. In some embodiments, the RET fusion polypeptide comprises an amino acid sequence encoded by a EEA1-RET fusion nucleic acid molecule of the disclosure that comprises or results from a breakpoint within chromosomal coordinates chr12: 93218451-93218763 and/or chr10: 43611264-43611592. In some embodiments, the RET fusion polypeptide comprises an amino acid sequence encoded by a CSGALNACT2-RET fusion nucleic acid molecule of the disclosure that comprises or results from a breakpoint within chromosomal coordinates chr10: 43671043-43671216 and/or chr10: 43611674-43611871. In some embodiments, the RET fusion polypeptide comprises an amino acid sequence encoded by a KIAA1217-RET fusion nucleic acid molecule of the disclosure that comprises or results from a breakpoint within chromosomal coordinates chr10: 24810035-24810374 and/or chr10: 43607337-43607677. In some embodiments, the RET fusion polypeptide comprises an amino acid sequence encoded by a SORBS1-RET fusion nucleic acid molecule of the disclosure that comprises or results from a breakpoint within chromosomal coordinates chr10: 97098893-97098933 and/or chr10: 43606666-43606706. In some embodiments, the RET fusion polypeptide comprises an amino acid sequence encoded by a SORBS1-RET fusion nucleic acid molecule of the disclosure that comprises or results from a breakpoint within chromosomal coordinates chr10: 97077294-97077537 and/or chr10: 43609629-43609972. In some embodiments, the RET fusion polypeptide comprises an amino acid sequence encoded by a MPRIP-RET fusion nucleic acid molecule of the disclosure that comprises or results from a breakpoint within chromosomal coordinates chr17: 17083314-17083354 and/or chr10: 43612074-43612114. In some embodiments, the RET fusion polypeptide comprises an amino acid sequence encoded by a TFG-RET fusion nucleic acid molecule of the disclosure that comprises or results from a breakpoint within chromosomal coordinates chr3: 100453395-100453864 and/or chr10: 43609629-43610065. In some embodiments, the RET fusion polypeptide comprises an amino acid sequence encoded by a SPECC1L-RET fusion nucleic acid molecule of the disclosure that comprises or results from a breakpoint within chromosomal coordinates chr22: 24739286-24739616 and/or chr10: 43611732-43612114. In some embodiments, the RET fusion polypeptide comprises an amino acid sequence encoded by a REEP3-RET fusion nucleic acid molecule of the disclosure that comprises or results from a breakpoint within chromosomal coordinates chr10: 65367361-65368009 and/or chr10: 43611264-43611758. In some embodiments, the RET fusion polypeptide comprises an amino acid sequence encoded by a RRBP1-RET fusion nucleic acid molecule of the disclosure that comprises or results from a breakpoint within chromosomal coordinates chr20: 17596315-17596767 and/or chr10: 43610785-43611339. In some embodiments, the RET fusion polypeptide comprises an amino acid sequence encoded by a ETV6-RET fusion nucleic acid molecule of the disclosure that comprises or results from a breakpoint within chromosomal coordinates chr12: 12038351-12038584 and/or chr10: 43611377-43611586. In some embodiments, the RET fusion polypeptide comprises an amino acid sequence encoded by a TAF3-RET fusion nucleic acid molecule of the disclosure that comprises or results from a breakpoint within chromosomal coordinates chr10: 8010776-8011037 and/or chr10: 43610288-43610507. In some embodiments, the RET fusion polypeptide comprises an amino acid sequence encoded by a PCM1-RET fusion nucleic acid molecule of the disclosure that comprises or results from a breakpoint within chromosomal coordinates chr8: 17856613-17856789 and/or chr10: 43611737-43612079. In some embodiments, the RET fusion polypeptide comprises an amino acid sequence encoded by a PCM1-RET fusion nucleic acid molecule of the disclosure that comprises or results from a breakpoint within chromosomal coordinates chr8: 17858876-17859548 and/or chr10: 43610288-43611131. In some embodiments, the RET fusion polypeptide comprises an amino acid sequence encoded by a TNIP2-RET fusion nucleic acid molecule of the disclosure that comprises or results from a breakpoint within chromosomal coordinates chr4: 2745391-2745953 and/or chr10: 43611294-43612177. In some embodiments, the RET fusion polypeptide comprises an amino acid sequence encoded by a SATB1-RET fusion nucleic acid molecule of the disclosure that comprises or results from a breakpoint within chromosomal coordinates chr3: 18433247-18433798 and/or chr10: 43610364-43611028. In some embodiments, the RET fusion polypeptide comprises an amino acid sequence encoded by a RET-ADCY1 fusion nucleic acid molecule of the disclosure that comprises or results from a breakpoint within chromosomal coordinates chr10: 43610900-43611038 and/or chr7: 45724501-45724758. In some embodiments, the RET fusion polypeptide comprises an amino acid sequence encoded by a RET-ZNF248 fusion nucleic acid molecule of the disclosure that comprises or results from a breakpoint within chromosomal coordinates chr10: 43610329-43610698 and/or chr10: 38143593-38143957. In some embodiments, the RET fusion polypeptide comprises an amino acid sequence encoded by a RET-AGBL4 fusion nucleic acid molecule of the disclosure that comprises or results from a breakpoint within chromosomal coordinates chr10: 43612164-43612322 and/or chr1: 50296066-50296164. In some embodiments, the RET fusion polypeptide comprises an amino acid sequence encoded by a RET-LRMDA fusion nucleic acid molecule of the disclosure that comprises or results from a breakpoint within chromosomal coordinates chr10: 43609359-43609695 and/or chr10: 78117734-78117976. In some embodiments, the RET fusion polypeptide comprises an amino acid sequence encoded by a RET-ARHGAP19 fusion nucleic acid molecule of the disclosure that comprises or results from a breakpoint within chromosomal coordinates chr10: 43610326-43610687 and/or chr10: 99014615-99014923. In some embodiments, the RET fusion polypeptide comprises an amino acid sequence encoded by a RET-CPEB3 fusion nucleic acid molecule of the disclosure that comprises or results from a breakpoint within chromosomal coordinates chr10: 43610292-43610477 and/or chr10: 93857438-93857889. In some embodiments, the RET fusion polypeptide comprises an amino acid sequence encoded by a RET-DCC fusion nucleic acid molecule of the disclosure that comprises or results from a breakpoint within chromosomal coordinates chr10: 43608372-43608512 and/or chr18: 50604698-50604780. In some embodiments, the RET fusion polypeptide comprises an amino acid sequence encoded by a RET-ELMO1 fusion nucleic acid molecule of the disclosure that comprises or results from a breakpoint within chromosomal coordinates chr10: 43611573-43611801 and/or chr7: 37238470-37238795. In some embodiments, the RET fusion polypeptide comprises an amino acid sequence encoded by a RET-WDFY4 fusion nucleic acid molecule of the disclosure that comprises or results from a breakpoint within chromosomal coordinates chr10: 43615421-43615713 and/or chr10: 50051602-50051938. In some embodiments, the RET fusion polypeptide comprises an amino acid sequence encoded by a MKX-RET fusion nucleic acid molecule of the disclosure that comprises or results from a breakpoint within chromosomal coordinates chr10: 27975034-27975183 and/or chr10: 43610285-43610466. In some embodiments, the RET fusion polypeptide comprises an amino acid sequence encoded by a RBMS3-RET fusion nucleic acid molecule of the disclosure that comprises or results from a breakpoint within chromosomal coordinates chr3: 29823809-29823976 and/or chr10: 43612021-43612151. In some embodiments, the RET fusion polypeptide comprises an amino acid sequence encoded by a SGIP1-RET fusion nucleic acid molecule of the disclosure that comprises or results from a breakpoint within chromosomal coordinates chr1: 67073312-67073582 and/or chr10: 43611993-43612185. In some embodiments, the RET fusion polypeptide comprises an amino acid sequence encoded by a ZSWIM6-RET fusion nucleic acid molecule of the disclosure that comprises or results from a breakpoint within chromosomal coordinates chr5: 60644190-60644600 and/or chr10: 43611931-43612183. In some embodiments, the RET fusion polypeptide comprises an amino acid sequence encoded by a ALOX5-RET fusion nucleic acid molecule of the disclosure that comprises or results from a breakpoint within chromosomal coordinates chr10: 45880054-45880377 and/or chr10: 43611435-43611795. In some embodiments, the RET fusion polypeptide comprises an amino acid sequence encoded by a RET-NTRK2 fusion nucleic acid molecule of the disclosure that comprises or results from a breakpoint within chromosomal coordinates chr10: 43609278-43609516 and/or chr9: 87615294-87615609. In some embodiments, the RET fusion polypeptide comprises an amino acid sequence encoded by a RET-OXR1 fusion nucleic acid molecule of the disclosure that comprises or results from a breakpoint within chromosomal coordinates chr10: 43611090-43611446 and/or chr8: 107581829-107582082. In some embodiments, the RET fusion polypeptide comprises an amino acid sequence encoded by a RET-CCBE1 fusion nucleic acid molecule of the disclosure that comprises or results from a breakpoint within chromosomal coordinates chr10: 43607774-43607874 and/or chr18: 57343203-57343676. In some embodiments, the RET fusion polypeptide comprises an amino acid sequence encoded by a RET-NAALADL2 fusion nucleic acid molecule of the disclosure that comprises or results from a breakpoint within chromosomal coordinates chr10: 43611602-43611922 and/or chr3: 175458489-175458778. In some embodiments, the RET fusion polypeptide comprises an amino acid sequence encoded by a RAI14-RET fusion nucleic acid molecule of the disclosure that comprises or results from a breakpoint within chromosomal coordinates chr5: 34825746-34825976 and/or chr10: 43610042-43610268. In some embodiments, the RET fusion polypeptide comprises an amino acid sequence encoded by a ABI3BP-RET fusion nucleic acid molecule of the disclosure that comprises or results from a breakpoint within chromosomal coordinates chr3: 100469166-100469527 and/or chr10: 43610324-43610920. In some embodiments, the RET fusion polypeptide comprises an amino acid sequence encoded by a LINC00379-RET fusion nucleic acid molecule of the disclosure that comprises or results from a breakpoint within chromosomal coordinates chr13: 91833445-91833706 and/or chr10: 43611105-43611505. In some embodiments, the RET fusion polypeptide comprises an amino acid sequence encoded by a OPTN-RET fusion nucleic acid molecule of the disclosure that comprises or results from a breakpoint within chromosomal coordinates chr10: 13145247-13145480 and/or chr10: 43609677-43609931. In some embodiments, the RET fusion polypeptide comprises an amino acid sequence encoded by a SH2D3A-RET fusion nucleic acid molecule of the disclosure that comprises or results from a breakpoint within chromosomal coordinates chr19: 6765967-6766439 and/or chr10: 43611764-43612170. In some embodiments, the RET fusion polypeptide comprises an amino acid sequence encoded by a ZNF721-RET fusion nucleic acid molecule of the disclosure that comprises or results from a breakpoint within chromosomal coordinates chr4: 472119-472287 and/or chr10: 43610406-43610589. In some embodiments, the RET fusion polypeptide comprises an amino acid sequence encoded by a ADAMTS14-RET fusion nucleic acid molecule of the disclosure that comprises or results from a breakpoint within chromosomal coordinates chr10: 72480354 and/or chr10: 43611720. In some embodiments, the RET fusion polypeptide comprises an amino acid sequence encoded by a CSGALNACT2-RET fusion nucleic acid molecule of the disclosure that comprises or results from a breakpoint within chromosomal coordinates chr10: 43648115-43648300 and/or chr10: 43610689-43610896. In some embodiments, the RET fusion polypeptide comprises an amino acid sequence encoded by a CPEB3-RET fusion nucleic acid molecule of the disclosure that comprises or results from a breakpoint within chromosomal coordinates chr10: 93808581-93808772 and/or chr10: 43611585-43611862. In some embodiments, the RET fusion polypeptide comprises an amino acid sequence encoded by a RET-CCNY fusion nucleic acid molecule of the disclosure that comprises or results from a breakpoint within chromosomal coordinates chr10: 43611301-43611576 and/or chr10: 35549015-35549283. In some embodiments, the RET fusion polypeptide comprises an amino acid sequence encoded by a RET-RASGEF1A fusion nucleic acid molecule of the disclosure that comprises or results from a breakpoint within chromosomal coordinates chr10: 43601756-43602031 and/or chr10: 43709383-43709498. In some embodiments, the RET fusion polypeptide comprises an amino acid sequence encoded by a RET-HSD17B7P2 fusion nucleic acid molecule of the disclosure that comprises or results from a breakpoint within chromosomal coordinates chr10: 43609878-43609957 and/or chr10: 38667312-38667485. In some embodiments, the RET fusion polypeptide comprises an amino acid sequence encoded by a RET-RAD1 fusion nucleic acid molecule of the disclosure that comprises or results from a breakpoint within chromosomal coordinates chr10: 43609716-43610036 and/or chr5: 34906366-34906711. In some embodiments, the RET fusion polypeptide comprises an amino acid sequence encoded by a RET-VSTM4 fusion nucleic acid molecule of the disclosure that comprises or results from a breakpoint within chromosomal coordinates chr10: 43609237-43609437 and/or chr10: 50225719-50225999. In some embodiments, the RET fusion polypeptide comprises an amino acid sequence encoded by an ANKRD26-RET fusion nucleic acid molecule of the disclosure that comprises or results from a breakpoint within chromosomal coordinates chr10: 27310264-27310361 and/or chr10: 43610383-43610480. In some embodiments, the RET fusion polypeptide comprises an amino acid sequence encoded by a CLIP1-RET fusion nucleic acid molecule of the disclosure that comprises or results from a breakpoint within chromosomal coordinates chr12: 122805302-122805302 and/or chr10: 43611109-43611109. In some embodiments, the RET fusion polypeptide comprises an amino acid sequence encoded by a DLG5-RET fusion nucleic acid molecule of the disclosure that comprises or results from a breakpoint within chromosomal coordinates chr10: 79582000-79582000 and/or chr10: 43611570-43611570. In some embodiments, the RET fusion polypeptide comprises an amino acid sequence encoded by a DLG5-RET fusion nucleic acid molecule of the disclosure that comprises or results from a breakpoint within chromosomal coordinates chr10: 79559942-79559942 and/or chr10: 43607485-43607485. In some embodiments, the RET fusion polypeptide comprises an amino acid sequence encoded by an ERC1-RET fusion nucleic acid molecule of the disclosure that comprises or results from a breakpoint within chromosomal coordinates chr12: 1346557-1346557 and/or chr10: 43610879-43610879. In some embodiments, the RET fusion polypeptide comprises an amino acid sequence encoded by an ERC1-RET fusion nucleic acid molecule of the disclosure that comprises or results from a breakpoint within chromosomal coordinates chr12: 1290627-1290627 and/or chr10: 43611066-43611066. In some embodiments, the RET fusion polypeptide comprises an amino acid sequence encoded by an ERC1-RET fusion nucleic acid molecule of the disclosure that comprises or results from a breakpoint within chromosomal coordinates chr12: 1350447-1350447 and/or chr10: 43609823-43609823. In some embodiments, the RET fusion polypeptide comprises an amino acid sequence encoded by a FRMD4A-RET fusion nucleic acid molecule of the disclosure that comprises or results from a breakpoint within chromosomal coordinates chr10: 13758406-13758406 and/or chr10: 43610354-43610354. In some embodiments, the RET fusion polypeptide comprises an amino acid sequence encoded by a KIAA1468-RET fusion nucleic acid molecule of the disclosure that comprises or results from a breakpoint within chromosomal coordinates chr18: 59908435-59908435 and/or chr10: 43611083-43611083. In some embodiments, the RET fusion polypeptide comprises an amino acid sequence encoded by a NCOA4-RET fusion nucleic acid molecule of the disclosure that comprises or results from a breakpoint within chromosomal coordinates chr10: 51585637-51585735 and/or chr10: 43610257-43610403. In some embodiments, the RET fusion polypeptide comprises an amino acid sequence encoded by a PARD3-RET fusion nucleic acid molecule of the disclosure that comprises or results from a breakpoint within chromosomal coordinates chr10: 34926445-34926445 and/or chr10: 43607138-43607138. In some embodiments, the RET fusion polypeptide comprises an amino acid sequence encoded by a PARD3-RET fusion nucleic acid molecule of the disclosure that comprises or results from a breakpoint within chromosomal coordinates chr10: 34755707-34755707 and/or chr10: 43612003-43612003. In some embodiments, the RET fusion polypeptide comprises an amino acid sequence encoded by a PRKAR1A-RET fusion nucleic acid molecule of the disclosure that comprises or results from a breakpoint within chromosomal coordinates chr17: 66523509-66523607 and/or chr10: 43611401-43611502. In some embodiments, the RET fusion polypeptide comprises an amino acid sequence encoded by a PRKG1-RET fusion nucleic acid molecule of the disclosure that comprises or results from a breakpoint within chromosomal coordinates chr10: 53889856-53889954 and/or chr10: 43600288-43600402. In some embodiments, the RET fusion polypeptide comprises an amino acid sequence encoded by a PRKG1-RET fusion nucleic acid molecule of the disclosure that comprises or results from a breakpoint within chromosomal coordinates chr10: 53789188-53789284 and/or chr10: 43620162-43620329. In some embodiments, the RET fusion polypeptide comprises an amino acid sequence encoded by a RUFY2-RET fusion nucleic acid molecule of the disclosure that comprises or results from a breakpoint within chromosomal coordinates chr10: 70143736-70143833 and/or chr10: 43611405-43611519. In some embodiments, the RET fusion polypeptide comprises an amino acid sequence encoded by a SNRNP70-RET fusion nucleic acid molecule of the disclosure that comprises or results from a breakpoint within chromosomal coordinates chr19: 49602640-49602640 and/or chr10: 43611323-43611323. In some embodiments, the RET fusion polypeptide comprises an amino acid sequence encoded by a SQSTM1-RET fusion nucleic acid molecule of the disclosure that comprises or results from a breakpoint within chromosomal coordinates chr5: 179254189-179254286 and/or chr10: 43609433-43609563. In some embodiments, the RET fusion polypeptide comprises an amino acid sequence encoded by a TNIP1-RET fusion nucleic acid molecule of the disclosure that comprises or results from a breakpoint within chromosomal coordinates chr5: 150416127-150416127 and/or chr10: 43611669-43611669. In some embodiments, the RET fusion polypeptide comprises an amino acid sequence encoded by a TNIP1-RET fusion nucleic acid molecule of the disclosure that comprises or results from a breakpoint within chromosomal coordinates chr5: 150414253-150414351 and/or chr10: 43611007-43611109. In some embodiments, the RET fusion polypeptide comprises an amino acid sequence encoded by a TNIP1-RET fusion nucleic acid molecule of the disclosure that comprises or results from a breakpoint within chromosomal coordinates chr5: 150417592-150417592 and/or chr10: 43611428-43611428. In some embodiments, the RET fusion polypeptide comprises an amino acid sequence encoded by a TRIM27-RET fusion nucleic acid molecule of the disclosure that comprises or results from a breakpoint within chromosomal coordinates chr6: 28881702-28881890 and/or chr10: 43610709-43610878. In some embodiments, the RET fusion polypeptide comprises an amino acid sequence encoded by a TRIM33-RET fusion nucleic acid molecule of the disclosure that comprises or results from a breakpoint within chromosomal coordinates chr1: 114965871-114965970 and/or chr10: 43611514-43611626. In some embodiments, the RET fusion polypeptide comprises an amino acid sequence encoded by a CCDC6-RET fusion nucleic acid molecule of the disclosure that comprises or results from a breakpoint within chromosomal coordinates chr10: 61585545-61585642 and/or chr10: 43609686-43609783. In some embodiments, the RET fusion polypeptide comprises an amino acid sequence encoded by a TRIM24-RET fusion nucleic acid molecule of the disclosure that comprises or results from a breakpoint within chromosomal coordinates chr7: 138267911-138267911 and/or chr10: 43611642-43611642. In some embodiments, the RET fusion polypeptide comprises an amino acid sequence encoded by a FGFR1OP-RET fusion nucleic acid molecule of the disclosure that comprises or results from a breakpoint within chromosomal coordinates chr6: 167424630-167424630 and/or chr10: 43611296-43611296. In some embodiments, the RET fusion polypeptide comprises an amino acid sequence encoded by a GAS2-RET fusion nucleic acid molecule of the disclosure that comprises or results from a breakpoint within chromosomal coordinates chr11: 22812170-22812170 and/or chr10: 43600385-43600385. In some embodiments, the RET fusion polypeptide comprises an amino acid sequence encoded by a HOOK1-RET fusion nucleic acid molecule of the disclosure that comprises or results from a breakpoint within chromosomal coordinates chr1: 60334563-60334563 and/or chr10: 43610286-43610286. In some embodiments, the RET fusion polypeptide comprises an amino acid sequence encoded by a LMNA-RET fusion nucleic acid molecule of the disclosure that comprises or results from a breakpoint within chromosomal coordinates chr1: 156102332-156102332 and/or chr10: 43611407-43611407. In some embodiments, the RET fusion polypeptide comprises an amino acid sequence encoded by a LMNA-RET fusion nucleic acid molecule of the disclosure that comprises or results from a breakpoint within chromosomal coordinates chr1: 156102293-156102386 and/or chr10: 43611365-43611462. In some embodiments, the RET fusion polypeptide comprises an amino acid sequence encoded by a MLPH-RET fusion nucleic acid molecule of the disclosure that comprises or results from a breakpoint within chromosomal coordinates chr2: 238445254-238445254 and/or chr10: 43610453-43610453. In some embodiments, the RET fusion polypeptide comprises an amino acid sequence encoded by a MYH9-RET fusion nucleic acid molecule of the disclosure that comprises or results from a breakpoint within chromosomal coordinates chr22: 36683797-36683797 and/or chr10: 43611848-43611848. In some embodiments, the RET fusion polypeptide comprises an amino acid sequence encoded by a PCDH15-RET fusion nucleic acid molecule of the disclosure that comprises or results from a breakpoint within chromosomal coordinates chr10: 56509282-56509282 and/or chr10: 43601819-43601819. In some embodiments, the RET fusion polypeptide comprises an amino acid sequence encoded by a PIBF1-RET fusion nucleic acid molecule of the disclosure that comprises or results from a breakpoint within chromosomal coordinates chr13: 73550847-73550847 and/or chr10: 43611532-43611532. In some embodiments, the RET fusion polypeptide comprises an amino acid sequence encoded by a PIBF1-RET fusion nucleic acid molecule of the disclosure that comprises or results from a breakpoint within chromosomal coordinates chr13: 73548007-73548007 and/or chr10: 43611167-43611167. In some embodiments, the RET fusion polypeptide comprises an amino acid sequence encoded by a SLC12A2-RET fusion nucleic acid molecule of the disclosure that comprises or results from a breakpoint within chromosomal coordinates chr5: 127496774-127496774 and/or chr10: 43611855-43611855. In some embodiments, the RET fusion polypeptide comprises an amino acid sequence encoded by a ZNF485-RET fusion nucleic acid molecule of the disclosure that comprises or results from a breakpoint within chromosomal coordinates chr10: 44109385-44109456 and/or chr10: 43611964-43612093. In some embodiments, the RET fusion polypeptide comprises an amino acid sequence encoded by a ZNF485-RET fusion nucleic acid molecule of the disclosure that comprises or results from a breakpoint within chromosomal coordinates chr10: 44109396-44109396 and/or chr10: 43608521-43608521.

In some embodiments, a RET fusion polypeptide of the disclosure comprises an amino acid sequence encoded by a RET fusion nucleic acid molecule of the disclosure that comprises or results from a fusion between a 5′ exon, or a portion thereof, and the corresponding 3′ exon, or a portion thereof, as indicated in Table 6. In some embodiments, the RET fusion polypeptide comprises an amino acid sequence encoded by a GOLGB1-RET fusion nucleic acid molecule of the disclosure that comprises or results from a fusion, in 5′ to 3′ direction, of GOLGB1 exon 10, or a portion thereof, fused to RET exon 12, or a portion thereof. In some embodiments, the RET fusion polypeptide comprises an amino acid sequence encoded by a GP2-RET fusion nucleic acid molecule of the disclosure that comprises or results from a fusion, in 5′ to 3′ direction, of GP2 exon 4, or a portion thereof, fused to RET exon11, or a portion thereof. In some embodiments, the RET fusion polypeptide comprises an amino acid sequence encoded by a PPP2R5A-RET fusion nucleic acid molecule of the disclosure that comprises or results from a fusion, in 5′ to 3′ direction, of PPP2R5A exon 1, or a portion thereof, fused to RET exon 20, or a portion thereof. In some embodiments, the RET fusion polypeptide comprises an amino acid sequence encoded by a GRM7-RET fusion nucleic acid molecule of the disclosure that comprises or results from a fusion, in 5′ to 3′ direction, of GRM7 exon 9, or a portion thereof, fused to RET exon 3, or a portion thereof. In some embodiments, the RET fusion polypeptide comprises an amino acid sequence encoded by a MYH14-RET fusion nucleic acid molecule of the disclosure that comprises or results from a fusion, in 5′ to 3′ direction, of MYH14 exon 37, or a portion thereof, fused to RET exon 12, or a portion thereof. In some embodiments, the RET fusion polypeptide comprises an amino acid sequence encoded by a MGEA5-RET fusion nucleic acid molecule of the disclosure that comprises or results from a fusion, in 5′ to 3′ direction, of MGEA5 exon 12, or a portion thereof, fused to RET exon 12, or a portion thereof. In some embodiments, the RET fusion polypeptide comprises an amino acid sequence encoded by a SAMD4A-RET fusion nucleic acid molecule of the disclosure that comprises or results from a fusion, in 5′ to 3′ direction, of SAMD4A exon 2, or a portion thereof, fused to RET exon 11, or a portion thereof. In some embodiments, the RET fusion polypeptide comprises an amino acid sequence encoded by a ACPP-RET fusion nucleic acid molecule of the disclosure that comprises or results from a fusion, in 5′ to 3′ direction, of ACPP exon 10, or a portion thereof, fused to RET exon11, or a portion thereof. In some embodiments, the RET fusion polypeptide comprises an amino acid sequence encoded by a BMS1-RET fusion nucleic acid molecule of the disclosure that comprises or results from a fusion, in 5′ to 3′ direction, of BMS1 exon 13, or a portion thereof, fused to RET exon 10, or a portion thereof. In some embodiments, the RET fusion polypeptide comprises an amino acid sequence encoded by a CEP135-RET fusion nucleic acid molecule of the disclosure that comprises or results from a fusion, in 5′ to 3′ direction, of CEP135 exon 11, or a portion thereof, fused to RET exon 12, or a portion thereof. In some embodiments, the RET fusion polypeptide comprises an amino acid sequence encoded by a EEA1-RET fusion nucleic acid molecule of the disclosure that comprises or results from a fusion, in 5′ to 3′ direction, of EEA1 exon 13, or a portion thereof, fused to RET exon 12, or a portion thereof. In some embodiments, the RET fusion polypeptide comprises an amino acid sequence encoded by a CSGALNACT2-RET fusion nucleic acid molecule of the disclosure that comprises or results from a fusion, in 5′ to 3′ direction, of CSGALNACT2 exon 6, or a portion thereof, fused to RET exon 12, or a portion thereof. In some embodiments, the RET fusion polypeptide comprises an amino acid sequence encoded by a KIAA1217-RET fusion nucleic acid molecule of the disclosure that comprises or results from a fusion, in 5′ to 3′ direction, of KIAA1217 exon 11, or a portion thereof, fused to RET exon8, or a portion thereof. In some embodiments, the RET fusion polypeptide comprises an amino acid sequence encoded by a SORBS1-RET fusion nucleic acid molecule of the disclosure that comprises or results from a fusion, in 5′ to 3′ direction, of SORBS1 exon 20, or a portion thereof, fused to RET exon7, or a portion thereof. In some embodiments, the RET fusion polypeptide comprises an amino acid sequence encoded by a SORBS1-RET fusion nucleic acid molecule of the disclosure that comprises or results from a fusion, in 5′ to 3′ direction, of SORBS1 exon 22, or a portion thereof, fused to RET exon 11, or a portion thereof. In some embodiments, the RET fusion polypeptide comprises an amino acid sequence encoded by a MPRIP-RET fusion nucleic acid molecule of the disclosure that comprises or results from a fusion, in 5′ to 3′ direction, of MPRIP exon 22, or a portion thereof, fused to RET exon 12, or a portion thereof. In some embodiments, the RET fusion polypeptide comprises an amino acid sequence encoded by a TFG-RET fusion nucleic acid molecule of the disclosure that comprises or results from a fusion, in 5′ to 3′ direction, of TFG exon 5, or a portion thereof, fused to RET exon 11, or a portion thereof. In some embodiments, the RET fusion polypeptide comprises an amino acid sequence encoded by a SPECC1L-RET fusion nucleic acid molecule of the disclosure that comprises or results from a fusion, in 5′ to 3′ direction, of SPECC1L exon 10, or a portion thereof, fused to RET exon 12, or a portion thereof. In some embodiments, the RET fusion polypeptide comprises an amino acid sequence encoded by a REEP3-RET fusion nucleic acid molecule of the disclosure that comprises or results from a fusion, in 5′ to 3′ direction, of REEP3 exon 5, or a portion thereof, fused to RET exon 12, or a portion thereof. In some embodiments, the RET fusion polypeptide comprises an amino acid sequence encoded by a RRBP1-RET fusion nucleic acid molecule of the disclosure that comprises or results from a fusion, in 5′ to 3′ direction, of RRBP1 exon 22, or a portion thereof, fused to RET exon 12, or a portion thereof. In some embodiments, the RET fusion polypeptide comprises an amino acid sequence encoded by a ETV6-RET fusion nucleic acid molecule of the disclosure that comprises or results from a fusion, in 5′ to 3′ direction, of ETV6 exon 6, or a portion thereof, fused to RET exon 12, or a portion thereof. In some embodiments, the RET fusion polypeptide comprises an amino acid sequence encoded by a TAF3-RET fusion nucleic acid molecule of the disclosure that comprises or results from a fusion, in 5′ to 3′ direction, of TAF3 exon 3, or a portion thereof, fused to RET exon 12, or a portion thereof. In some embodiments, the RET fusion polypeptide comprises an amino acid sequence encoded by a PCM1-RET fusion nucleic acid molecule of the disclosure that comprises or results from a fusion, in 5′ to 3′ direction, of PCM1 exon 29, or a portion thereof, fused to RET exon 12, or a portion thereof. In some embodiments, the RET fusion polypeptide comprises an amino acid sequence encoded by a PCM1-RET fusion nucleic acid molecule of the disclosure that comprises or results from a fusion, in 5′ to 3′ direction, of PCM1 exon 29, or a portion thereof, fused to RET exon 12, or a portion thereof. In some embodiments, the RET fusion polypeptide comprises an amino acid sequence encoded by a TNIP2-RET fusion nucleic acid molecule of the disclosure that comprises or results from a fusion, in 5′ to 3′ direction, of TNIP2 exon 5, or a portion thereof, fused to RET exon 12, or a portion thereof. In some embodiments, the RET fusion polypeptide comprises an amino acid sequence encoded by a SATB1-RET fusion nucleic acid molecule of the disclosure that comprises or results from a fusion, in the 5′ to 3′ direction, of SATB1 exon 7, or a portion thereof, fused to RET exon 12, or a portion thereof. In some embodiments, the RET fusion polypeptide comprises an amino acid sequence encoded by a RET-ADCY1 fusion nucleic acid molecule of the disclosure that comprises or results from a fusion, in 5′ to 3′ direction, of RET exon 11, or a portion thereof, fused to ADCY1 exon 12, or a portion thereof. In some embodiments, the RET fusion polypeptide comprises an amino acid sequence encoded by a RET-ZNF248 fusion nucleic acid molecule of the disclosure that comprises or results from a fusion, in 5′ to 3′ direction, of RET exon 11, or a portion thereof, fused to ZNF248 exon 4, or a portion thereof. In some embodiments, the RET fusion polypeptide comprises an amino acid sequence encoded by a RET-AGBL4 fusion nucleic acid molecule of the disclosure that comprises or results from a fusion, in 5′ to 3′ direction, of RET exon 12, or a portion thereof, fused to AGBL4 exon 3, or a portion thereof. In some embodiments, the RET fusion polypeptide comprises an amino acid sequence encoded by a RET-LRMDA fusion nucleic acid molecule of the disclosure that comprises or results from a fusion, in 5′ to 3′ direction, of RET exon 10, or a portion thereof, fused to LRMDA exon 6, or a portion thereof. In some embodiments, the RET fusion polypeptide comprises an amino acid sequence encoded by a RET-ARHGAP19 fusion nucleic acid molecule of the disclosure that comprises or results from a fusion, in 5′ to 3′ direction, of RET exon 11, or a portion thereof, fused to ARHGAP19 exon 7, or a portion thereof. In some embodiments, the RET fusion polypeptide comprises an amino acid sequence encoded by a RET-CPEB3 fusion nucleic acid molecule of the disclosure that comprises or results from a fusion, in the 5′ to 3′ direction, of RET exon 11, or a portion thereof, fused to CPEB3 exon 8, or a portion thereof. In some embodiments, the RET fusion polypeptide comprises an amino acid sequence encoded by a RET-DCC fusion nucleic acid molecule of the disclosure that comprises or results from a fusion, in the 5′ to 3′ direction, of RET exon 9, or a portion thereof, fused to DCC exon 8, or a portion thereof. In some embodiments, the RET fusion polypeptide comprises an amino acid sequence encoded by a RET-ELMO1 fusion nucleic acid molecule of the disclosure that comprises or results from a fusion, in 5′ to 3′ direction, of RET exon 11, or a portion thereof, fused to ELMO1 exon 14, or a portion thereof. In some embodiments, the RET fusion polypeptide comprises an amino acid sequence encoded by a RET-WDFY4 fusion nucleic acid molecule of the disclosure that comprises or results from a fusion, in 5′ to 3′ direction, of RET exon 15, or a portion thereof, fused to WDFY4 exon 40, or a portion thereof. In some embodiments, the RET fusion polypeptide comprises an amino acid sequence encoded by a MKX-RET fusion nucleic acid molecule of the disclosure that comprises or results from a fusion, in 5′ to 3′ direction, of MKX exon 5, or a portion thereof, fused to RET exon 12, or a portion thereof. In some embodiments, the RET fusion polypeptide comprises an amino acid sequence encoded by a RBMS3-RET fusion nucleic acid molecule of the disclosure that comprises or results from a fusion, in 5′ to 3′ direction, of RBMS3 exon 6, or a portion thereof, fused to RET exon 12, or a portion thereof. In some embodiments, the RET fusion polypeptide comprises an amino acid sequence encoded by a SGIP1-RET fusion nucleic acid molecule of the disclosure that comprises or results from a fusion, in 5′ to 3′ direction, of SGIP1 exon 1, or a portion thereof, fused to RET exon 12, or a portion thereof. In some embodiments, the RET fusion polypeptide comprises an amino acid sequence encoded by a ZSWIM6-RET fusion nucleic acid molecule of the disclosure that comprises or results from a fusion, in 5′ to 3′ direction, of ZSWIM6 exon 1, or a portion thereof, fused to RET exon 12, or a portion thereof. In some embodiments, the RET fusion polypeptide comprises an amino acid sequence encoded by a ALOX5-RET fusion nucleic acid molecule of the disclosure that comprises or results from a fusion, in 5′ to 3′ direction, of ALOX5 exon 2, or a portion thereof, fused to RET exon 12, or a portion thereof. In some embodiments, the RET fusion polypeptide comprises an amino acid sequence encoded by a RET-NTRK2 fusion nucleic acid molecule of the disclosure that comprises or results from a fusion, in the 5′ to 3′ direction, of RET exon 10, or a portion thereof, fused to NTRK2 exon 20, or a portion thereof. In some embodiments, the RET fusion polypeptide comprises an amino acid sequence encoded by a RET-OXR1 fusion nucleic acid molecule of the disclosure that comprises or results from a fusion, in 5′ to 3′ direction, of RET exon 11, or a portion thereof, fused to OXR1 exon 4, or a portion thereof. In some embodiments, the RET fusion polypeptide comprises an amino acid sequence encoded by a RET-CCBE1 fusion nucleic acid molecule of the disclosure that comprises or results from a fusion, in 5′ to 3′ direction, of RET exon 8, or a portion thereof, fused to CCBE1 exon 3, or a portion thereof. In some embodiments, the RET fusion polypeptide comprises an amino acid sequence encoded by a RET-NAALADL2 fusion nucleic acid molecule of the disclosure that comprises or results from a fusion, in 5′ to 3′ direction, of RET exon 11, or a portion thereof, fused to NAALADL2 exon 13, or a portion thereof. In some embodiments, the RET fusion polypeptide comprises an amino acid sequence encoded by a RAI14-RET fusion nucleic acid molecule of the disclosure that comprises or results from a fusion, in 5′ to 3′ direction, of RAI14 exon 15, or a portion thereof, fused to RET exon11, or a portion thereof. In some embodiments, the RET fusion polypeptide comprises an amino acid sequence encoded by a ABI3BP-RET fusion nucleic acid molecule of the disclosure that comprises or results from a fusion, in 5′ to 3′ direction, of ABI3BP exon 35, or a portion thereof, fused to RET exon 12, or a portion thereof. In some embodiments, the RET fusion polypeptide comprises an amino acid sequence encoded by a LINC00379-RET fusion nucleic acid molecule of the disclosure that comprises or results from a fusion, in 5′ to 3′ direction, of LINC00379 exon 2, or a portion thereof, fused to RET exon 12, or a portion thereof. In some embodiments, the RET fusion polypeptide comprises an amino acid sequence encoded by a OPTN-RET fusion nucleic acid molecule of the disclosure that comprises or results from a fusion, in 5′ to 3′ direction, of OPTN exon 1, or a portion thereof, fused to RET exon11, or a portion thereof. In some embodiments, the RET fusion polypeptide comprises an amino acid sequence encoded by a SH2D3A-RET fusion nucleic acid molecule of the disclosure that comprises or results from a fusion, in 5′ to 3′ direction, of SH2D3A exon 1, or a portion thereof, fused to RET exon 12, or a portion thereof. In some embodiments, the RET fusion polypeptide comprises an amino acid sequence encoded by a ZNF721-RET fusion nucleic acid molecule of the disclosure that comprises or results from a fusion, in 5′ to 3′ direction, of ZNF721 exon 1, or a portion thereof, fused to RET exon 12, or a portion thereof. In some embodiments, the RET fusion polypeptide comprises an amino acid sequence encoded by a ADAMTS14-RET fusion nucleic acid molecule of the disclosure that comprises or results from a fusion, in 5′ to 3′ direction, of ADAMTS14 exon 4, or a portion thereof, fused to RET exon 12, or a portion thereof. In some embodiments, the RET fusion polypeptide comprises an amino acid sequence encoded by a CSGALNACT2-RET fusion nucleic acid molecule of the disclosure that comprises or results from a fusion, in 5′ to 3′ direction, of CSGALNACT2 exon 1, or a portion thereof, fused to RET exon 12, or a portion thereof. In some embodiments, the RET fusion polypeptide comprises an amino acid sequence encoded by a CPEB3-RET fusion nucleic acid molecule of the disclosure that comprises or results from a fusion, in 5′ to 3′ direction, of CPEB3 exon 10, or a portion thereof, fused to RET exon 12, or a portion thereof. In some embodiments, the RET fusion polypeptide comprises an amino acid sequence encoded by a RET-CCNY fusion nucleic acid molecule of the disclosure that comprises or results from a fusion, in 5′ to 3′ direction, of RET exon 11, or a portion thereof, fused to CCNY exon 4, or a portion thereof. In some embodiments, the RET fusion polypeptide comprises an amino acid sequence encoded by a RET-RASGEF1A fusion nucleic acid molecule of the disclosure that comprises or results from a fusion, in 5′ to 3′ direction, of RET exon 5, or a portion thereof, fused to RASGEF1A exon 2, or a portion thereof. In some embodiments, the RET fusion polypeptide comprises an amino acid sequence encoded by a RET-HSD17B7P2 fusion nucleic acid molecule of the disclosure that comprises or results from a fusion, in 5′ to 3′ direction, of RET exon 11, or a portion thereof, fused to HSD17B7P2 exon 8, or a portion thereof. In some embodiments, the RET fusion polypeptide comprises an amino acid sequence encoded by a RET-RAD1 fusion nucleic acid molecule of the disclosure that comprises or results from a fusion, in 5′ to 3′ direction, of RET exon 11, or a portion thereof, fused to RAD1 exon 6, or a portion thereof. In some embodiments, the RET fusion polypeptide comprises an amino acid sequence encoded by a RET-VSTM4 fusion nucleic acid molecule of the disclosure that comprises or results from a fusion, in 5′ to 3′ direction, of RET exon 10, or a portion thereof, fused to VSTM4 exon 8, or a portion thereof. In some embodiments, the RET fusion polypeptide comprises an amino acid sequence encoded by an ANKRD26-RET fusion nucleic acid molecule of the disclosure that comprises or results from a fusion, in 5′ to 3′ direction, of ANKRD26 exon 29, or a portion thereof, fused to RET exon 12, or a portion thereof. In some embodiments, the RET fusion polypeptide comprises an amino acid sequence encoded by a CLIP1-RET fusion nucleic acid molecule of the disclosure that comprises or results from a fusion, in 5′ to 3′ direction, of CLIP1 exon 16, or a portion thereof, fused to RET exon 12, or a portion thereof. In some embodiments, the RET fusion polypeptide comprises an amino acid sequence encoded by a DLG5-RET fusion nucleic acid molecule of the disclosure that comprises or results from a fusion, in 5′ to 3′ direction, of DLG5 exon 14, or a portion thereof, fused to RET exon 12, or a portion thereof. In some embodiments, the RET fusion polypeptide comprises an amino acid sequence encoded by a DLG5-RET fusion nucleic acid molecule of the disclosure that comprises or results from a fusion, in 5′ to 3′ direction, of DLG5 exon 27, or a portion thereof, fused to RET exon 8, or a portion thereof. In some embodiments, the RET fusion polypeptide comprises an amino acid sequence encoded by an ERC1-RET fusion nucleic acid molecule of the disclosure that comprises or results from a fusion, in 5′ to 3′ direction, of ERC1 exon 12, or a portion thereof, fused to RET exon 12, or a portion thereof. In some embodiments, the RET fusion polypeptide comprises an amino acid sequence encoded by an ERC1-RET fusion nucleic acid molecule of the disclosure that comprises or results from a fusion, in 5′ to 3′ direction, of ERC1 exon 8, or a portion thereof, fused to RET exon 12, or a portion thereof. In some embodiments, the RET fusion polypeptide comprises an amino acid sequence encoded by an ERC1-RET fusion nucleic acid molecule of the disclosure that comprises or results from a fusion, in 5′ to 3′ direction, of ERC1 exon 12, or a portion thereof, fused to RET exon 11, or a portion thereof. In some embodiments, the RET fusion polypeptide comprises an amino acid sequence encoded by a FRMD4A-RET fusion nucleic acid molecule of the disclosure that comprises or results from a fusion, in 5′ to 3′ direction, of FRMD4A exon 12, or a portion thereof, fused to RET exon 12, or a portion thereof. In some embodiments, the RET fusion polypeptide comprises an amino acid sequence encoded by a KIAA1468-RET fusion nucleic acid molecule of the disclosure that comprises or results from a fusion, in 5′ to 3′ direction, of KIAA1468 exon 10, or a portion thereof, fused to RET exon 12, or a portion thereof. In some embodiments, the RET fusion polypeptide comprises an amino acid sequence encoded by a NCOA4-RET fusion nucleic acid molecule of the disclosure that comprises or results from a fusion, in 5′ to 3′ direction, of NCOA4 exon 8, or a portion thereof, fused to RET exon 12, or a portion thereof. In some embodiments, the RET fusion polypeptide comprises an amino acid sequence encoded by a PARD3-RET fusion nucleic acid molecule of the disclosure that comprises or results from a fusion, in 5′ to 3′ direction, of PARD3 exon 2, or a portion thereof, fused to RET exon 8, or a portion thereof. In some embodiments, the RET fusion polypeptide comprises an amino acid sequence encoded by a PARD3-RET fusion nucleic acid molecule of the disclosure that comprises or results from a fusion, in 5′ to 3′ direction, of PARD3 exon 4, or a portion thereof, fused to RET exon 12, or a portion thereof. In some embodiments, the RET fusion polypeptide comprises an amino acid sequence encoded by a PRKAR1A-RET fusion nucleic acid molecule of the disclosure that comprises or results from a fusion, in 5′ to 3′ direction, of PRKAR1A exon 7, or a portion thereof, fused to RET exon 12, or a portion thereof. In some embodiments, the RET fusion polypeptide comprises an amino acid sequence encoded by a PRKG1-RET fusion nucleic acid molecule of the disclosure that comprises or results from a fusion, in 5′ to 3′ direction, of PRKG1 exon 7, or a portion thereof, fused to RET exon 4, or a portion thereof. In some embodiments, the RET fusion polypeptide comprises an amino acid sequence encoded by a PRKG1-RET fusion nucleic acid molecule of the disclosure that comprises or results from a fusion, in the 5′ to 3′ direction, of PRKG1 exon 5, or a portion thereof, fused to RET exon 18, or a portion thereof. In some embodiments, the RET fusion polypeptide comprises an amino acid sequence encoded by a RUFY2-RET fusion nucleic acid molecule of the disclosure that comprises or results from a fusion, in 5′ to 3′ direction, of RUFY2 exon 9, or a portion thereof, fused to RET exon 12, or a portion thereof. In some embodiments, the RET fusion polypeptide comprises an amino acid sequence encoded by a SNRNP70-RET fusion nucleic acid molecule of the disclosure that comprises or results from a fusion, in 5′ to 3′ direction, of SNRNP70 exon 6, or a portion thereof, fused to RET exon 12, or a portion thereof. In some embodiments, the RET fusion polypeptide comprises an amino acid sequence encoded by a SQSTM1-RET fusion nucleic acid molecule of the disclosure that comprises or results from a fusion, in 5′ to 3′ direction, of SQSTM1 exon 5, or a portion thereof, fused to RET exon 11, or a portion thereof. In some embodiments, the RET fusion polypeptide comprises an amino acid sequence encoded by a TNIP1-RET fusion nucleic acid molecule of the disclosure that comprises or results from a fusion, in 5′ to 3′ direction, of TNIP1 exon 13, or a portion thereof, fused to RET exon 12, or a portion thereof. In some embodiments, the RET fusion polypeptide comprises an amino acid sequence encoded by a TNIP1-RET fusion nucleic acid molecule of the disclosure that comprises or results from a fusion, in 5′ to 3′ direction, of TNIP1 exon 15, or a portion thereof, fused to RET exon 12, or a portion thereof. In some embodiments, the RET fusion polypeptide comprises an amino acid sequence encoded by a TNIP1-RET fusion nucleic acid molecule of the disclosure that comprises or results from a fusion, in 5′ to 3′ direction, of TNIP1 exon 12, or a portion thereof, fused to RET exon 12, or a portion thereof. In some embodiments, the RET fusion polypeptide comprises an amino acid sequence encoded by a TRIM27-RET fusion nucleic acid molecule of the disclosure that comprises or results from a fusion, in 5′ to 3′ direction, of TRIM27 exon 3, or a portion thereof, fused to RET exon 12, or a portion thereof. In some embodiments, the RET fusion polypeptide comprises an amino acid sequence encoded by a TRIM33-RET fusion nucleic acid molecule of the disclosure that comprises or results from a fusion, in 5′ to 3′ direction, of TRIM33 exon 10, or a portion thereof, fused to RET exon 12, or a portion thereof. In some embodiments, the RET fusion polypeptide comprises an amino acid sequence encoded by a CCDC6-RET fusion nucleic acid molecule of the disclosure that comprises or results from a fusion, in 5′ to 3′ direction, of CCDC6 exon 3, or a portion thereof, fused to RET exon 11, or a portion thereof. In some embodiments, the RET fusion polypeptide comprises an amino acid sequence encoded by a TRIM24-RET fusion nucleic acid molecule of the disclosure that comprises or results from a fusion, in 5′ to 3′ direction, of TRIM24 exon 17, or a portion thereof, fused to RET exon 12, or a portion thereof. In some embodiments, the RET fusion polypeptide comprises an amino acid sequence encoded by a FGFR1OP-RET fusion nucleic acid molecule of the disclosure that comprises or results from a fusion, in 5′ to 3′ direction, of FGFR1OP exon 6, or a portion thereof, fused to RET exon 12, or a portion thereof. In some embodiments, the RET fusion polypeptide comprises an amino acid sequence encoded by a GAS2-RET fusion nucleic acid molecule of the disclosure that comprises or results from a fusion, in 5′ to 3′ direction, of GAS2 exon 6, or a portion thereof, fused to RET exon 4, or a portion thereof. In some embodiments, the RET fusion polypeptide comprises an amino acid sequence encoded by a HOOK1-RET fusion nucleic acid molecule of the disclosure that comprises or results from a fusion, in 5′ to 3′ direction, of HOOK1 exon 20, or a portion thereof, fused to RET exon 12, or a portion thereof. In some embodiments, the RET fusion polypeptide comprises an amino acid sequence encoded by a LMNA-RET fusion nucleic acid molecule of the disclosure that comprises or results from a fusion, in 5′ to 3′ direction, of LMNA exon 2, or a portion thereof, fused to RET exon 12, or a portion thereof. In some embodiments, the RET fusion polypeptide comprises an amino acid sequence encoded by a MLPH-RET fusion nucleic acid molecule of the disclosure that comprises or results from a fusion, in 5′ to 3′ direction, of MLPH exon 9, or a portion thereof, fused to RET exon 12, or a portion thereof. In some embodiments, the RET fusion polypeptide comprises an amino acid sequence encoded by a MYH9-RET fusion nucleic acid molecule of the disclosure that comprises or results from a fusion, in the 5′ to 3′ direction, of MYH9 exon 34, or a portion thereof, fused to RET exon 12, or a portion thereof. In some embodiments, the RET fusion polypeptide comprises an amino acid sequence encoded by a PCDH15-RET fusion nucleic acid molecule of the disclosure that comprises or results from a fusion, in 5′ to 3′ direction, of PCDH15 exon 1, or a portion thereof, fused to RET exon 5, or a portion thereof. In some embodiments, the RET fusion polypeptide comprises an amino acid sequence encoded by a PIBF1-RET fusion nucleic acid molecule of the disclosure that comprises or results from a fusion, in 5′ to 3′ direction, of PIBF1 exon 16, or a portion thereof, fused to RET exon 12, or a portion thereof. In some embodiments, the RET fusion polypeptide comprises an amino acid sequence encoded by a SLC12A2-RET fusion nucleic acid molecule of the disclosure that comprises or results from a fusion, in 5′ to 3′ direction, of SLC12A2 exon 16, or a portion thereof, fused to RET exon 12, or a portion thereof. In some embodiments, the RET fusion polypeptide comprises an amino acid sequence encoded by a ZNF485-RET fusion nucleic acid molecule of the disclosure that comprises or results from a fusion, in 5′ to 3′ direction, of ZNF485 exon 4, or a portion thereof, fused to RET exon 12, or a portion thereof. In some embodiments, the RET fusion polypeptide comprises an amino acid sequence encoded by a ZNF485-RET fusion nucleic acid molecule of the disclosure that comprises or results from a fusion, in 5′ to 3′ direction, of ZNF485 exon 4, or a portion thereof, fused to RET exon 10, or a portion thereof.

In some embodiments, a RET fusion polypeptide of the disclosure comprises an amino acid sequence encoded by a RET fusion nucleic acid molecule of the disclosure that comprises, in 5′ to 3′ direction, the corresponding exons or portions thereof as listed in Table 7. In some embodiments, the RET fusion polypeptide comprises an amino acid sequence encoded by a GOLGB1-RET fusion nucleic acid molecule of the disclosure that comprises, in 5′ to 3′ direction, exons 1-9, and exon 10 or a portion thereof, of GOLGB1 fused to exon 12 or a portion thereof, and exons 13-19, of RET. In some embodiments, the RET fusion polypeptide comprises an amino acid sequence encoded by a GP2-RET fusion nucleic acid molecule of the disclosure that comprises, in 5′ to 3′ direction, exons 1-3, and exon 4 or a portion thereof, of GP2 fused to exon 11 or a portion thereof, and exons 12-19, of RET. In some embodiments, the RET fusion polypeptide comprises an amino acid sequence encoded by a PPP2R5A-RET fusion nucleic acid molecule of the disclosure that comprises, in 5′ to 3′ direction, exon 1 or a portion thereof, of PPP2R5A fused to exon 20 or a portion thereof, of RET. In some embodiments, the RET fusion polypeptide comprises an amino acid sequence encoded by a GRM7-RET fusion nucleic acid molecule of the disclosure that comprises, in 5′ to 3′ direction, exons 1-8, and exon 9 or a portion thereof, of GRM7 fused to exon 3 or a portion thereof, and exons 4-19, of RET. In some embodiments, the RET fusion polypeptide comprises an amino acid sequence encoded by a MYH14-RET fusion nucleic acid molecule of the disclosure that comprises, in the 5′ to 3′ direction, exons 1-36, and exon 37 or a portion thereof, of MYH14 fused to exon 12 or a portion thereof, and exons 13-19, of RET. In some embodiments, the RET fusion polypeptide comprises an amino acid sequence encoded by a MGEA5-RET fusion nucleic acid molecule of the disclosure that comprises, in 5′ to 3′ direction, exons 1-11, and exon 12 or a portion thereof, of MGEA5 fused to exon 12 or a portion thereof, and exons 13-19, of RET. In some embodiments, the RET fusion polypeptide comprises an amino acid sequence encoded by a SAMD4A-RET fusion nucleic acid molecule of the disclosure that comprises, in 5′ to 3′ direction, exon 1, and exon 2 or a portion thereof, of SAMD4A fused to exon 11 or a portion thereof, and exons 12-19, of RET. In some embodiments, the RET fusion polypeptide comprises an amino acid sequence encoded by a ACPP-RET fusion nucleic acid molecule of the disclosure that comprises, in 5′ to 3′ direction, exons 1-9, and exon 10 or a portion thereof, of ACPP fused to exon 11 or a portion thereof, and exons 12-19, of RET. In some embodiments, the RET fusion polypeptide comprises an amino acid sequence encoded by a BMS1-RET fusion nucleic acid molecule of the disclosure that comprises, in 5′ to 3′ direction, exons 1-12, and exon 13 or a portion thereof, of BMS1 fused to exon 10 or a portion thereof, and exons 11-19, of RET. In some embodiments, the RET fusion polypeptide comprises an amino acid sequence encoded by a CEP135-RET fusion nucleic acid molecule of the disclosure that comprises, in 5′ to 3′ direction, exons 1-10, and exon 11 or a portion thereof, of CEP135 fused to exon 12 or a portion thereof, and exons 13-19, of RET. In some embodiments, the RET fusion polypeptide comprises an amino acid sequence encoded by a EEA1-RET fusion nucleic acid molecule of the disclosure that comprises, in 5′ to 3′ direction, exons 1-12, and exon 13 or a portion thereof, of EEA1 fused to exon 12 or a portion thereof, and exons 13-19, of RET. In some embodiments, the RET fusion polypeptide comprises an amino acid sequence encoded by a CSGALNACT2-RET fusion nucleic acid molecule of the disclosure that comprises, in 5′ to 3′ direction, exons 1-5, and exon 6 or a portion thereof, of CSGALNACT2 fused to exon 12 or a portion thereof, and exons 13-19, of RET. In some embodiments, the RET fusion polypeptide comprises an amino acid sequence encoded by a KIAA1217-RET fusion nucleic acid molecule of the disclosure that comprises, in 5′ to 3′ direction, exons 1-10, and exon 11 or a portion thereof, of KIAA1217 fused to exon 8 or a portion thereof, and exons 9-19, of RET. In some embodiments, the RET fusion polypeptide comprises an amino acid sequence encoded by a SORBS1-RET fusion nucleic acid molecule of the disclosure that comprises, in 5′ to 3′ direction, exons 1-19, and exon 20 or a portion thereof, of SORBS1 fused to exon 7 or a portion thereof, and exons 8-19, of RET. In some embodiments, the RET fusion polypeptide comprises an amino acid sequence encoded by a SORBS1-RET fusion nucleic acid molecule of the disclosure that comprises, in 5′ to 3′ direction, exons 1-21, and exon 22 or a portion thereof, of SORBS1 fused to exon 11 or a portion thereof, and exons 12-19, of RET. In some embodiments, the RET fusion polypeptide comprises an amino acid sequence encoded by a MPRIP-RET fusion nucleic acid molecule of the disclosure that comprises, in 5′ to 3′ direction, exons 1-21, and exon 22 or a portion thereof, of MPRIP fused to exon 12 or a portion thereof, and exons 13-19, of RET. In some embodiments, the RET fusion polypeptide comprises an amino acid sequence encoded by a TFG-RET fusion nucleic acid molecule of the disclosure that comprises, in 5′ to 3″ direction, exons 1-4, and exon 5 or a portion thereof, of TFG fused to exon 11 or a portion thereof, and exons 12-19, of RET. In some embodiments, the RET fusion polypeptide comprises an amino acid sequence encoded by a SPECC1L-RET fusion nucleic acid molecule of the disclosure that comprises, in 5′ to 3′ direction, exons 1-9, and exon 10 or a portion thereof, of SPECC1L fused to exon 12 or a portion thereof, and exons 13-19, of RET. In some embodiments, the RET fusion polypeptide comprises an amino acid sequence encoded by a REEP3-RET fusion nucleic acid molecule of the disclosure that comprises, in 5′ to 3′ direction, exons 1-4, and exon 5 or a portion thereof, of REEP3 fused to exon 12 or a portion thereof, and exons 13-19, of RET. In some embodiments, the RET fusion polypeptide comprises an amino acid sequence encoded by a RRBP1-RET fusion nucleic acid molecule of the disclosure that comprises, in 5′ to 3′ direction, exons 1-21, and exon 22 or a portion thereof, of RRBP1 fused to exon 12 or a portion thereof, and exons 13-19, of RET. In some embodiments, the RET fusion polypeptide comprises an amino acid sequence encoded by a ETV6-RET fusion nucleic acid molecule of the disclosure that comprises, in 5′ to 3″ direction, exons 1-5, and exon 6 or a portion thereof, of ETV6 fused to exon 12 or a portion thereof, and exons 13-19, of RET. In some embodiments, the RET fusion polypeptide comprises an amino acid sequence encoded by a TAF3-RET fusion nucleic acid molecule of the disclosure that comprises, in 5′ to 3′ direction, exons 1-2, and exon 3 or a portion thereof, of TAF3 fused to exon 12 or a portion thereof, and exons 13-19, of RET. In some embodiments, the RET fusion polypeptide comprises an amino acid sequence encoded by a PCM1-RET fusion nucleic acid molecule of the disclosure that comprises, in 5′ to 3′ direction, exons 1-28, and exon 29 or a portion thereof, of PCM1 fused to exon 12 or a portion thereof, and exons 13-19, of RET. In some embodiments, the RET fusion polypeptide comprises an amino acid sequence encoded by a TNIP2-RET fusion nucleic acid molecule of the disclosure that comprises, in 5′ to 3′ direction, exons 1-4, and exon 5 or a portion thereof, of TNIP2 fused to exon 12 or a portion thereof, and exons 13-19, of RET. In some embodiments, the RET fusion polypeptide comprises an amino acid sequence encoded by a SATB1-RET fusion nucleic acid molecule of the disclosure that comprises, in 5′ to 3′ direction, exons 1-6, and exon 7 or a portion thereof, of SATB1 fused to exon 12 or a portion thereof, and exons 13-19, of RET. In some embodiments, the RET fusion polypeptide comprises an amino acid sequence encoded by a RET-ADCY1 fusion nucleic acid molecule of the disclosure that comprises, in 5′ to 3′ direction, exons 1-10, and exon 11 or a portion thereof, of RET fused to exon 12 or a portion thereof, and exons 13-20, of ADCY1. In some embodiments, the RET fusion polypeptide comprises an amino acid sequence encoded by a RET-ZNF248 fusion nucleic acid molecule of the disclosure that comprises, in 5′ to 3′ direction, exons 1-10, and exon 11 or a portion thereof, of RET fused to exon 4 or a portion thereof, and exons 5-6, of ZNF248. In some embodiments, the RET fusion polypeptide comprises an amino acid sequence encoded by a RET-AGBL4 fusion nucleic acid molecule of the disclosure that comprises, in 5′ to 3′ direction, exons 1-11, and exon 12 or a portion thereof, of RET fused to exon 3 or a portion thereof, and exons 4-14, of AGBL4. In some embodiments, the RET fusion polypeptide comprises an amino acid sequence encoded by a RET-LRMDA fusion nucleic acid molecule of the disclosure that comprises, in 5′ to 3′ direction, exons 1-9, and exon 10 or a portion thereof, of RET fused to exon 6 or a portion thereof, of LRMDA. In some embodiments, the RET fusion polypeptide comprises an amino acid sequence encoded by a RET-ARHGAP19 fusion nucleic acid molecule of the disclosure that comprises, in 5′ to 3′ direction, exons 1-10, and exon 11 or a portion thereof, of RET fused to exon 7 or a portion thereof, and exons 8-12, of ARHGAP19. In some embodiments, the RET fusion polypeptide comprises an amino acid sequence encoded by a RET-CPEB3 fusion nucleic acid molecule of the disclosure that comprises, in 5′ to 3′ direction, exons 1-10, and exon 11 or a portion thereof, of RET fused to exon 8 or a portion thereof, and exons 9-10, of CPEB3. In some embodiments, the RET fusion polypeptide comprises an amino acid sequence encoded by a RET-DCC fusion nucleic acid molecule of the disclosure that comprises, in 5′ to 3′ direction, exons 1-8, and exon 9 or a portion thereof, of RET fused to exon 8 or a portion thereof, and exons 9-29, of DCC. In some embodiments, the RET fusion polypeptide comprises an amino acid sequence encoded by a RET-ELMO1 fusion nucleic acid molecule of the disclosure that comprises, in 5′ to 3′ direction, exons 1-10, and exon 11 or a portion thereof, of RET fused to exon 14 or a portion thereof, and exons 15-22, of ELMO1. In some embodiments, the RET fusion polypeptide comprises an amino acid sequence encoded by a RET-WDFY4 fusion nucleic acid molecule of the disclosure that comprises, in 5′ to 3′ direction, exons 1-14, and exon 15 or a portion thereof, of RET fused to exon 40 or a portion thereof, and exons 41-62, of WDFY4. In some embodiments, the RET fusion polypeptide comprises an amino acid sequence encoded by a MKX-RET fusion nucleic acid molecule of the disclosure that comprises, in 5′ to 3″ direction, exons 1-4, and exon 5 or a portion thereof, of MKX fused to exon 12 or a portion thereof, and exons 13-19, of RET. In some embodiments, the RET fusion polypeptide comprises an amino acid sequence encoded by a RBMS3-RET fusion nucleic acid molecule of the disclosure that comprises, in 5′ to 3′ direction, exons 1-5, and exon 6 or a portion thereof, of RBMS3 fused to exon 12 or a portion thereof, and exons 13-19, of RET. In some embodiments, the RET fusion polypeptide comprises an amino acid sequence encoded by a SGIP1-RET fusion nucleic acid molecule of the disclosure that comprises, in 5′ to 3′ direction, exon 1 or a portion thereof, of SGIP1 fused to exon 12 or a portion thereof, and exons 13-19, of RET. In some embodiments, the RET fusion polypeptide comprises an amino acid sequence encoded by a ZSWIM6-RET fusion nucleic acid molecule of the disclosure that comprises, in 5′ to 3′ direction, exon 1 or a portion thereof, of ZSWIM6 fused to exon 12 or a portion thereof, and exons 13-19, of RET. In some embodiments, the RET fusion polypeptide comprises an amino acid sequence encoded by a ALOX5-RET fusion nucleic acid molecule of the disclosure that comprises, in 5′ to 3′ direction, exon 1, and exon 2 or a portion thereof, of ALOX5 fused to exon 12 or a portion thereof, and exons 13-19, of RET. In some embodiments, the RET fusion polypeptide comprises an amino acid sequence encoded by a RET-NTRK2 fusion nucleic acid molecule of the disclosure that comprises, in 5′ to 3′ direction, exons 1-9, and exon 10 or a portion thereof, of RET fused to exon 20 or a portion thereof, and exon 21, of NTRK2. In some embodiments, the RET fusion polypeptide comprises an amino acid sequence encoded by a RET-OXR1 fusion nucleic acid molecule of the disclosure that comprises, in the 5′ to 3′ direction, exons 1-10, and exon 11 or a portion thereof, of RET fused to exon 4 or a portion thereof, and exons 5-16, of OXR1. In some embodiments, the RET fusion polypeptide comprises an amino acid sequence encoded by a RET-CCBE1 fusion nucleic acid molecule of the disclosure that comprises, in 5′ to 3′ direction, exons 1-7, and exon 8 or a portion thereof, of RET fused to exon 3 or a portion thereof, and exons 4-11, of CCBE1. In some embodiments, the RET fusion polypeptide comprises an amino acid sequence encoded by a RET-NAALADL2 fusion nucleic acid molecule of the disclosure that comprises, in 5′ to 3′ direction, exons 1-10, and exon 11 or a portion thereof, of RET fused to exon 13 or a portion thereof, and exon 14, of NAALADL2. In some embodiments, the RET fusion polypeptide comprises an amino acid sequence encoded by a RAI14-RET fusion nucleic acid molecule of the disclosure that comprises, in 5′ to 3′ direction, exons 1-14, and exon 15 or a portion thereof, of RAI14 fused to exon 11 or a portion thereof, and exons 12-19, of RET. In some embodiments, the RET fusion polypeptide comprises an amino acid sequence encoded by a ABI3BP-RET fusion nucleic acid molecule of the disclosure that comprises, in 5′ to 3′ direction, exons 1-34, and exon 35 or a portion thereof, of ABI3BP fused to exon 12 or a portion thereof, and exons 13-19, of RET. In some embodiments, the RET fusion polypeptide comprises an amino acid sequence encoded by a LINC00379-RET fusion nucleic acid molecule of the disclosure that comprises, in 5′ to 3′ direction, exon 1, and exon 2 or a portion thereof, of LINC00379 fused to exon 12 or a portion thereof, and exons 13-19, of RET. In some embodiments, the RET fusion polypeptide comprises an amino acid sequence encoded by a OPTN-RET fusion nucleic acid molecule of the disclosure that comprises, in 5′ to 3′ direction, exon 1 or a portion thereof, of OPTN fused to exon 11 or a portion thereof, and exons 12-19, of RET. In some embodiments, the RET fusion polypeptide comprises an amino acid sequence encoded by a SH2D3A-RET fusion nucleic acid molecule of the disclosure that comprises, in 5′ to 3′ direction, exon 1 or a portion thereof, of SH2D3A fused to exon 12 or a portion thereof, and exons 13-19, of RET. In some embodiments, the RET fusion polypeptide comprises an amino acid sequence encoded by a ZNF721-RET fusion nucleic acid molecule of the disclosure that comprises, in 5′ to 3′ direction, exon 1 or a portion thereof, of ZNF721 fused to exon 12 or a portion thereof, and exons 13-19, of RET. In some embodiments, the RET fusion polypeptide comprises an amino acid sequence encoded by a ADAMTS14-RET fusion nucleic acid molecule of the disclosure that comprises, in 5′ to 3′ direction, exons 1-3, and exon 4 or a portion thereof, of ADAMTS14 fused to exon 12 or a portion thereof, and exons 13-19, of RET. In some embodiments, the RET fusion polypeptide comprises an amino acid sequence encoded by a CSGALNACT2-RET fusion nucleic acid molecule of the disclosure that comprises, in 5′ to 3′ direction, exon 1 or a portion thereof, of CSGALNACT2 fused to exon 12 or a portion thereof, and exons 13-19, of RET. In some embodiments, the RET fusion polypeptide comprises an amino acid sequence encoded by a CPEB3-RET fusion nucleic acid molecule of the disclosure that comprises, in 5′ to 3′ direction, exons 1-9, and exon 10 or a portion thereof, of CPEB3 fused to exon 12 or a portion thereof, and exons 13-19, of RET. In some embodiments, the RET fusion polypeptide comprises an amino acid sequence encoded by a RET-CCNY fusion nucleic acid molecule of the disclosure that comprises, in 5′ to 3′ direction, exons 1-10, and exon 11 or a portion thereof, of RET fused to exon 4 or a portion thereof, and exons 5-12, of CCNY. In some embodiments, the RET fusion polypeptide comprises an amino acid sequence encoded by a RET-RASGEF1A fusion nucleic acid molecule of the disclosure that comprises, in the 5′ to 3′ direction, exons 1-4, and exon 5 or a portion thereof, of RET fused to exon 2 or a portion thereof, and exons 3-13, of RASGEF1A. In some embodiments, the RET fusion polypeptide comprises an amino acid sequence encoded by a RET-HSD17B7P2 fusion nucleic acid molecule of the disclosure that comprises, in 5′ to 3′ direction, exons 1-10, and exon 11 or a portion thereof, of RET fused to exon 8 or a portion thereof, of HSD17B7P2. In some embodiments, the RET fusion polypeptide comprises an amino acid sequence encoded by a RET-RAD1 fusion nucleic acid molecule of the disclosure that comprises, in 5′ to 3′ direction, exons 1-10, and exon 11 or a portion thereof, of RET fused to exon 6 or a portion thereof, of RAD1. In some embodiments, the RET fusion polypeptide comprises an amino acid sequence encoded by a RET-VSTM4 fusion nucleic acid molecule of the disclosure that comprises, in 5′ to 3′ direction, exons 1-9, and exon 10 or a portion thereof, of RET fused to exon 8 or a portion thereof, of VSTM4. In some embodiments, the RET fusion polypeptide comprises an amino acid sequence encoded by a ANKRD26-RET fusion nucleic acid molecule of the disclosure that comprises, in 5′ to 3′ direction, exons 1-28, and exon 29 or a portion thereof, of ANKRD26 fused to exon 12 or a portion thereof, and exons 13-19, of RET. In some embodiments, the RET fusion polypeptide comprises an amino acid sequence encoded by a CLIP1-RET fusion nucleic acid molecule of the disclosure that comprises, in 5′ to 3′ direction, exons 1-15, and exon 16 or a portion thereof, of CLIP1 fused to exon 12 or a portion thereof, and exons 13-19, of RET. In some embodiments, the RET fusion polypeptide comprises an amino acid sequence encoded by a DLG5-RET fusion nucleic acid molecule of the disclosure that comprises, in the 5′ to 3′ direction, exons 1-13, and exon 14 or a portion thereof, of DLG5 fused to exon 12 or a portion thereof, and exons 13-19, of RET. In some embodiments, the RET fusion polypeptide comprises an amino acid sequence encoded by a DLG5-RET fusion nucleic acid molecule of the disclosure that comprises, in 5′ to 3′ direction, exons 1-26, and exon 27 or a portion thereof, of DLG5 fused to exon 8 or a portion thereof, and exons 9-19, of RET. In some embodiments, the RET fusion polypeptide comprises an amino acid sequence encoded by a ERC1-RET fusion nucleic acid molecule of the disclosure that comprises, in 5′ to 3′ direction, exons 1-11, and exon 12 or a portion thereof, of ERC1 fused to exon 12 or a portion thereof, and exons 13-19, of RET. In some embodiments, the RET fusion polypeptide comprises an amino acid sequence encoded by a ERC1-RET fusion nucleic acid molecule of the disclosure that comprises, in 5′ to 3′ direction, exons 1-7, and exon 8 or a portion thereof, of ERC1 fused to exon 12 or a portion thereof, and exons 13-19, of RET. In some embodiments, the RET fusion polypeptide comprises an amino acid sequence encoded by a ERC1-RET fusion nucleic acid molecule of the disclosure that comprises, in 5′ to 3′ direction, exons 1-11, and exon 12 or a portion thereof, of ERC1 fused to exon 11 or a portion thereof, and exons 12-19, of RET. In some embodiments, the RET fusion polypeptide comprises an amino acid sequence encoded by a FRMD4A-RET fusion nucleic acid molecule of the disclosure that comprises, in 5′ to 3′ direction, exons 1-11, and exon 12 or a portion thereof, of FRMD4A fused to exon 12 or a portion thereof, and exons 13-19, of RET. In some embodiments, the RET fusion polypeptide comprises an amino acid sequence encoded by a KIAA1468-RET fusion nucleic acid molecule of the disclosure that comprises, in 5′ to 3′ direction, exons 1-9, and exon 10 or a portion thereof, of KIAA1468 fused to exon 12 or a portion thereof, and exons 13-19, of RET. In some embodiments, the RET fusion polypeptide comprises an amino acid sequence encoded by a NCOA4-RET fusion nucleic acid molecule of the disclosure that comprises, in 5′ to 3′ direction, exons 1-7, and exon 8 or a portion thereof, of NCOA4 fused to exon 12 or a portion thereof, and exons 13-19, of RET. In some embodiments, the RET fusion polypeptide comprises an amino acid sequence encoded by a PARD3-RET fusion nucleic acid molecule of the disclosure that comprises, in the 5′ to 3′ direction, exon 1, and exon 2 or a portion thereof, of PARD3 fused to exon 8 or a portion thereof, and exons 9-19, of RET. In some embodiments, the RET fusion polypeptide comprises an amino acid sequence encoded by a PARD3-RET fusion nucleic acid molecule of the disclosure that comprises, in 5′ to 3′ direction, exons 1-3, and exon 4 or a portion thereof, of PARD3 fused to exon 12 or a portion thereof, and exons 13-19, of RET. In some embodiments, the RET fusion polypeptide comprises an amino acid sequence encoded by a PRKAR1A-RET fusion nucleic acid molecule of the disclosure that comprises, in 5′ to 3′ direction, exons 1-6, and exon 7 or a portion thereof, of PRKAR1A fused to exon 12 or a portion thereof, and exons 13-19, of RET. In some embodiments, the RET fusion polypeptide comprises an amino acid sequence encoded by a PRKG1-RET fusion nucleic acid molecule of the disclosure that comprises, in 5′ to 3′ direction, exons 1-6, and exon 7 or a portion thereof, of PRKG1 fused to exon 4 or a portion thereof, and exons 5-19, of RET. In some embodiments, the RET fusion polypeptide comprises an amino acid sequence encoded by a PRKG1-RET fusion nucleic acid molecule of the disclosure that comprises, in 5′ to 3″ direction, exons 1-4, and exon 5 or a portion thereof, of PRKG1 fused to exon 18 or a portion thereof, and exon 19, of RET. In some embodiments, the RET fusion polypeptide comprises an amino acid sequence encoded by a RUFY2-RET fusion nucleic acid molecule of the disclosure that comprises, in the 5′ to 3′ direction, exons 1-8, and exon 9 or a portion thereof, of RUFY2 fused to exon 12 or a portion thereof, and exons 13-19, of RET. In some embodiments, the RET fusion polypeptide comprises an amino acid sequence encoded by a SNRNP70-RET fusion nucleic acid molecule of the disclosure that comprises, in 5′ to 3′ direction, exons 1-5, and exon 6 or a portion thereof, of SNRNP70 fused to exon 12 or a portion thereof, and exons 13-19, of RET. In some embodiments, the RET fusion polypeptide comprises an amino acid sequence encoded by a SQSTM1-RET fusion nucleic acid molecule of the disclosure that comprises, in 5′ to 3′ direction, exons 1-4, and exon 5 or a portion thereof, of SQSTM1 fused to exon 11 or a portion thereof, and exons 12-19, of RET. In some embodiments, the RET fusion polypeptide comprises an amino acid sequence encoded by a TNIP1-RET fusion nucleic acid molecule of the disclosure that comprises, in 5′ to 3′ direction, exons 1-12, and exon 13 or a portion thereof, of TNIP1 fused to exon 12 or a portion thereof, and exons 13-19, of RET. In some embodiments, the RET fusion polypeptide comprises an amino acid sequence encoded by a TNIP1-RET fusion nucleic acid molecule of the disclosure that comprises, in the 5′ to 3′ direction, exons 1-14, and exon 15 or a portion thereof, of TNIP1 fused to exon 12 or a portion thereof, and exons 13-19, of RET. In some embodiments, the RET fusion polypeptide comprises an amino acid sequence encoded by a TNIP1-RET fusion nucleic acid molecule of the disclosure that comprises, in 5′ to 3′ direction, exons 1-11, and exon 12 or a portion thereof, of TNIP1 fused to exon 12 or a portion thereof, and exons 13-19, of RET. In some embodiments, the RET fusion polypeptide comprises an amino acid sequence encoded by a TRIM27-RET fusion nucleic acid molecule of the disclosure that comprises, in 5′ to 3′ direction, exons 1-2, and exon 3 or a portion thereof, of TRIM27 fused to exon 12 or a portion thereof, and exons 13-19, of RET. In some embodiments, the RET fusion polypeptide comprises an amino acid sequence encoded by a TRIM33-RET fusion nucleic acid molecule of the disclosure that comprises, in 5′ to 3′ direction, exons 1-9, and exon 10 or a portion thereof, of TRIM33 fused to exon 12 or a portion thereof, and exons 13-19, of RET. In some embodiments, the RET fusion polypeptide comprises an amino acid sequence encoded by a CCDC6-RET fusion nucleic acid molecule of the disclosure that comprises, in the 5′ to 3′ direction, exons 1-2, and exon 3 or a portion thereof, of CCDC6 fused to exon 11 or a portion thereof, and exons 12-19, of RET. In some embodiments, the RET fusion polypeptide comprises an amino acid sequence encoded by a TRIM24-RET fusion nucleic acid molecule of the disclosure that comprises, in 5′ to 3′ direction, exons 1-16, and exon 17 or a portion thereof, of TRIM24 fused to exon 12 or a portion thereof, and exons 13-19, of RET. In some embodiments, the RET fusion polypeptide comprises an amino acid sequence encoded by a FGFR1OP-RET fusion nucleic acid molecule of the disclosure that comprises, in 5′ to 3′ direction, exons 1-5, and exon 6 or a portion thereof, of FGFR1OP fused to exon 12 or a portion thereof, and exons 13-19, of RET. In some embodiments, the RET fusion polypeptide comprises an amino acid sequence encoded by a GAS2-RET fusion nucleic acid molecule of the disclosure that comprises, in 5′ to 3′ direction, exons 1-5, and exon 6 or a portion thereof, of GAS2 fused to exon 4 or a portion thereof, and exons 5-19, of RET. In some embodiments, the RET fusion polypeptide comprises an amino acid sequence encoded by a HOOK1-RET fusion nucleic acid molecule of the disclosure that comprises, in 5′ to 3′ direction, exons 1-19, and exon 20 or a portion thereof, of HOOK1 fused to exon 12 or a portion thereof, and exons 13-19, of RET. In some embodiments, the RET fusion polypeptide comprises an amino acid sequence encoded by a LMNA-RET fusion nucleic acid molecule of the disclosure that comprises, in 5′ to 3′ direction, exon 1, and exon 2 or a portion thereof, of LMNA fused to exon 12 or a portion thereof, and exons 13-19, of RET. In some embodiments, the RET fusion polypeptide comprises an amino acid sequence encoded by a MLPH-RET fusion nucleic acid molecule of the disclosure that comprises, in 5′ to 3′ direction, exons 1-8, and exon 9 or a portion thereof, of MLPH fused to exon 12 or a portion thereof, and exons 13-19, of RET. In some embodiments, the RET fusion polypeptide comprises an amino acid sequence encoded by a MYH9-RET fusion nucleic acid molecule of the disclosure that comprises, in 5′ to 3′ direction, exons 1-33, and exon 34 or a portion thereof, of MYH9 fused to exon 12 or a portion thereof, and exons 13-19, of RET. In some embodiments, the RET fusion polypeptide comprises an amino acid sequence encoded by a PCDH15-RET fusion nucleic acid molecule of the disclosure that comprises, in 5′ to 3′ direction, exon 1 or a portion thereof, of PCDH15 fused to exon 5 or a portion thereof, and exons 6-19, of RET. In some embodiments, the RET fusion polypeptide comprises an amino acid sequence encoded by a PIBF1-RET fusion nucleic acid molecule of the disclosure that comprises, in 5′ to 3′ direction, exons 1-15, and exon 16 or a portion thereof, of PIBF1 fused to exon 12 or a portion thereof, and exons 13-19, of RET. In some embodiments, the RET fusion polypeptide comprises an amino acid sequence encoded by a SLC12A2-RET fusion nucleic acid molecule of the disclosure that comprises, in 5′ to 3′ direction, exons 1-15, and exon 16 or a portion thereof, of SLC12A2 fused to exon 12 or a portion thereof, and exons 13-19, of RET. In some embodiments, the RET fusion polypeptide comprises an amino acid sequence encoded by a ZNF485-RET fusion nucleic acid molecule of the disclosure that comprises, in 5′ to 3′ direction, exons 1-3, and exon 4 or a portion thereof, of ZNF485 fused to exon 12 or a portion thereof, and exons 13-19, of RET. In some embodiments, the RET fusion polypeptide comprises an amino acid sequence encoded by a ZNF485-RET fusion nucleic acid molecule of the disclosure that comprises, in 5′ to 3′ direction, exons 1-3, and exon 4 or a portion thereof, of ZNF485 fused to exon 10 or a portion thereof, and exons 11-19, of RET.

In some embodiments, a RET fusion polypeptide of the disclosure comprises an amino acid sequence encoded by a RET fusion nucleic acid molecule of the disclosure that comprises the corresponding nucleotide sequence as listed in Table 8, or a nucleotide sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a GOLGB1-RET fusion nucleic acid molecule of the disclosure that comprises the nucleotide sequence of SEQ ID NO: 2, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a GP2-RET fusion nucleic acid molecule of the disclosure that comprises the nucleotide sequence of SEQ ID NO: 3, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a PPP2RSA-RET fusion nucleic acid molecule of the disclosure that comprises the nucleotide sequence of SEQ ID NO: 4, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a GRM7-RET fusion nucleic acid molecule of the disclosure that comprises the nucleotide sequence of SEQ ID NO: 6, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a MYH14-RET fusion nucleic acid molecule of the disclosure that comprises the nucleotide sequence of SEQ ID NO: 8, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a MGEA5-RET fusion nucleic acid molecule of the disclosure that comprises the nucleotide sequence of SEQ ID NO: 9, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a SAMD4A-RET fusion nucleic acid molecule of the disclosure that comprises the nucleotide sequence of SEQ ID NO: 10, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a ACPP-RET fusion nucleic acid molecule of the disclosure that comprises the nucleotide sequence of SEQ ID NO: 11, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a BMS1-RET fusion nucleic acid molecule of the disclosure that comprises the nucleotide sequence of SEQ ID NO: 12, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a CEP135-RET fusion nucleic acid molecule of the disclosure that comprises the nucleotide sequence of SEQ ID NO: 13, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a EEA1-RET fusion nucleic acid molecule of the disclosure that comprises the nucleotide sequence of SEQ ID NO: 14, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a CSGALNACT2-RET fusion nucleic acid molecule of the disclosure that comprises the nucleotide sequence of SEQ ID NO: 15, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a KIAA1217-RET fusion nucleic acid molecule of the disclosure that comprises the nucleotide sequence of SEQ ID NO: 16, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a SORBS1-RET fusion nucleic acid molecule of the disclosure that comprises the nucleotide sequence of SEQ ID NO: 17, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a SORBS1-RET fusion nucleic acid molecule of the disclosure that comprises the nucleotide sequence of SEQ ID NO: 18, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a MPRIP-RET fusion nucleic acid molecule of the disclosure that comprises the nucleotide sequence of SEQ ID NO: 19, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a TFG-RET fusion nucleic acid molecule of the disclosure that comprises the nucleotide sequence of SEQ ID NO: 20, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a SPECC1L-RET fusion nucleic acid molecule of the disclosure that comprises the nucleotide sequence of SEQ ID NO: 21, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a REEP3-RET fusion nucleic acid molecule of the disclosure that comprises the nucleotide sequence of SEQ ID NO: 22, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a RRBP1-RET fusion nucleic acid molecule of the disclosure that comprises the nucleotide sequence of SEQ ID NO: 23, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a ETV6-RET fusion nucleic acid molecule of the disclosure that comprises the nucleotide sequence of SEQ ID NO: 24, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a TAF3-RET fusion nucleic acid molecule of the disclosure that comprises the nucleotide sequence of SEQ ID NO: 25, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a PCM1-RET fusion nucleic acid molecule of the disclosure that comprises the nucleotide sequence of SEQ ID NO: 26, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a PCM1-RET fusion nucleic acid molecule of the disclosure that comprises the nucleotide sequence of SEQ ID NO: 27, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a TNIP2-RET fusion nucleic acid molecule of the disclosure that comprises the nucleotide sequence of SEQ ID NO: 28, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a SATB1-RET fusion nucleic acid molecule of the disclosure that comprises the nucleotide sequence of SEQ ID NO: 29, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a RET-ADCY1 fusion nucleic acid molecule of the disclosure that comprises the nucleotide sequence of SEQ ID NO: 30, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a RET-ZNF248 fusion nucleic acid molecule of the disclosure that comprises the nucleotide sequence of SEQ ID NO: 31, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a RET-AGBL4 fusion nucleic acid molecule of the disclosure that comprises the nucleotide sequence of SEQ ID NO: 32, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a RET-LRMDA fusion nucleic acid molecule of the disclosure that comprises the nucleotide sequence of SEQ ID NO: 33, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a RET-ARHGAP19 fusion nucleic acid molecule of the disclosure that comprises the nucleotide sequence of SEQ ID NO: 34, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a RET-CPEB3 fusion nucleic acid molecule of the disclosure that comprises the nucleotide sequence of SEQ ID NO: 35, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a RET-DCC fusion nucleic acid molecule of the disclosure that comprises the nucleotide sequence of SEQ ID NO: 36, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a RET-ELMO1 fusion nucleic acid molecule of the disclosure that comprises the nucleotide sequence of SEQ ID NO: 37, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a RET-WDFY4 fusion nucleic acid molecule of the disclosure that comprises the nucleotide sequence of SEQ ID NO: 38, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by an ANKRD26-RET fusion nucleic acid molecule of the disclosure that comprises the nucleotide sequence of SEQ ID NO: 39, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a CLIP1-RET fusion nucleic acid molecule of the disclosure that comprises the nucleotide sequence of SEQ ID NO: 40, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a DLG5-RET fusion nucleic acid molecule of the disclosure that comprises the nucleotide sequence of SEQ ID NO: 41, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a DLG5-RET fusion nucleic acid molecule of the disclosure that comprises the nucleotide sequence of SEQ ID NO: 42, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by an ERC1-RET fusion nucleic acid molecule of the disclosure that comprises the nucleotide sequence of SEQ ID NO: 43, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by an ERC1-RET fusion nucleic acid molecule of the disclosure that comprises the nucleotide sequence of SEQ ID NO: 44, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by an ERC1-RET fusion nucleic acid molecule of the disclosure that comprises the nucleotide sequence of SEQ ID NO: 45, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a FRMD4A-RET fusion nucleic acid molecule of the disclosure that comprises the nucleotide sequence of SEQ ID NO: 46, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a KIAA1468-RET fusion nucleic acid molecule of the disclosure that comprises the nucleotide sequence of SEQ ID NO: 47, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a NCOA4-RET fusion nucleic acid molecule of the disclosure that comprises the nucleotide sequence of SEQ ID NO: 48, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a PARD3-RET fusion nucleic acid molecule of the disclosure that comprises the nucleotide sequence of SEQ ID NO: 49, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a PARD3-RET fusion nucleic acid molecule of the disclosure that comprises the nucleotide sequence of SEQ ID NO: 50, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a PRKAR1A-RET fusion nucleic acid molecule of the disclosure that comprises the nucleotide sequence of SEQ ID NO: 51, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a PRKG1-RET fusion nucleic acid molecule of the disclosure that comprises the nucleotide sequence of SEQ ID NO: 52, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a PRKG1-RET fusion nucleic acid molecule of the disclosure that comprises the nucleotide sequence of SEQ ID NO: 53, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a RUFY2-RET fusion nucleic acid molecule of the disclosure that comprises the nucleotide sequence of SEQ ID NO: 54, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a SNRNP70-RET fusion nucleic acid molecule of the disclosure that comprises the nucleotide sequence of SEQ ID NO: 55, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a SQSTM1-RET fusion nucleic acid molecule of the disclosure that comprises the nucleotide sequence of SEQ ID NO: 56, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a TNIP1-RET fusion nucleic acid molecule of the disclosure that comprises the nucleotide sequence of SEQ ID NO: 57, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a TNIP1-RET fusion nucleic acid molecule of the disclosure that comprises the nucleotide sequence of SEQ ID NO: 58, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a TNIP1-RET fusion nucleic acid molecule of the disclosure that comprises the nucleotide sequence of SEQ ID NO: 59, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a TRIM27-RET fusion nucleic acid molecule of the disclosure that comprises the nucleotide sequence of SEQ ID NO: 60, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a TRIM33-RET fusion nucleic acid molecule of the disclosure that comprises the nucleotide sequence of SEQ ID NO: 61, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a CCDC6-RET fusion nucleic acid molecule of the disclosure that comprises the nucleotide sequence of SEQ ID NO: 62, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a TRIM24-RET fusion nucleic acid molecule of the disclosure that comprises the nucleotide sequence of SEQ ID NO: 63, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a FGFR1OP-RET fusion nucleic acid molecule of the disclosure that comprises the nucleotide sequence of SEQ ID NO: 64, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a GAS2-RET fusion nucleic acid molecule of the disclosure that comprises the nucleotide sequence of SEQ ID NO: 65, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a HOOK1-RET fusion nucleic acid molecule of the disclosure that comprises the nucleotide sequence of SEQ ID NO: 66, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a LMNA-RET fusion nucleic acid molecule of the disclosure that comprises the nucleotide sequence of SEQ ID NO: 147, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a LMNA-RET fusion nucleic acid molecule of the disclosure that comprises the nucleotide sequence of SEQ ID NO: 148, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a MLPH-RET fusion nucleic acid molecule of the disclosure that comprises the nucleotide sequence of SEQ ID NO: 149, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a MYH9-RET fusion nucleic acid molecule of the disclosure that comprises the nucleotide sequence of SEQ ID NO: 150, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a PCDH15-RET fusion nucleic acid molecule of the disclosure that comprises the nucleotide sequence of SEQ ID NO: 151, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a PIBF1-RET fusion nucleic acid molecule of the disclosure that comprises the nucleotide sequence of SEQ ID NO: 152, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a PIBF1-RET fusion nucleic acid molecule of the disclosure that comprises the nucleotide sequence of SEQ ID NO: 153, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a SLC12A2-RET fusion nucleic acid molecule of the disclosure that comprises the nucleotide sequence of SEQ ID NO: 154, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a ZNF485-RET fusion nucleic acid molecule of the disclosure that comprises the nucleotide sequence of SEQ ID NO: 155, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide comprises an amino acid sequence encoded by a ZNF485-RET fusion nucleic acid molecule of the disclosure that comprises the nucleotide sequence of SEQ ID NO: 156, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto.

In some embodiments, a RET fusion polypeptide of the disclosure comprises the corresponding amino acid sequence as listed in Table 9, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto.

TABLE 9
Exemplary RET fusion polypeptide amino acid sequences.

| RET Fusion | | SEQ |
|---|---|---|
| Polypeptide | Fusion Polypeptide Amino Acid Sequence | ID NO |
| GOLGB1-RET | MLSRLSGLANVVLHELSGDDDTDQNMRAPLDPELHQESDMEENNTTQED | 68 |
| | VQERLAYAEQLVVELKDIIRQKDVQLQQKDEALQEERKAADNKIKKLKL | |
| | HAKAKLTSLNKYIEEMKAQGGTVLPTEPQSEEQLSKHDKSSTEEEMEIE | |
| | KIKHKLQEKEELISTLQAQLTQAQAEQPAQSSTEMEEFVMMKQQLQEKE | |
| | EFISTLQAQLSQTQAEQAAQQVVREKDARFETQVRLHEDELLQLVTQAD | |
| | VETEMQQKLRVLQRKLEEHEESLVGRAQVVDLLQQELTAAEQRNQILSQ | |
| | QLQQMEAEHNTLRNTVETEREESKILLEKMELEVAERKLSFHNLQEEMH | |
| | HLLEQFEQAGQAQAELESRYSALEQKHKAEMEEKTSHILSLQKTGQELQ | |
| | SACDALKDQNSKLLQDKNEQAVQSAQTIQQLEDQLQQKSKEISQFLNRL | |
| | PLQQHETASQTSFPDVYNEGTQEDPKWEFPRKNLVLGKTLGEGEFGKVV | |
| | KATAFHLKGRAGYTTVAVKMLKENASPSELRDLLSEFNVLKQVNHPHVI | |
| | KLYGACSQDGPLLLIVEYAKYGSLRGELRESRKVGPGYLGSGGSRNSSS | |
| | LDHPDERALTMGDLISFAWQISQGMQYLAEMKLVHRDLAARNILVAEGR | |
| | KMKISDFGLSRDVYEEDSYVKRSQGRIPVKWMAIESLFDHIYTTQSDVW | |
| | SFGVLLWEIVTLGGNPYPGIPPERLENLLKTGHRMERPDNCSEEMYRLM | |
| | LQCWKQEPDKRPVFADISKDLEKMMVKRRDYLDLAASTPSDSLIYDDGL | |
| | SEEETPLVDCNNAPLPRALPSTWIENKLYGRISHAFTRF | |
| GP2-RET | MPHLMERMVGSGLLWLALVSCILTQASAVQRGYGNPIEASSYGLDLDCG | 69 |
| | APGTPEAHVCFDPCQNYTLLDEPFRSTENSAGSQGCDKNMSGWYRFVGE | |
| | GGVRMSETCVQVHRCQTDAPMWLNGTHPALGDGITNHTACAHWSGNCCF | |
| | WKTEVLVKACPGGYHVYRLEGTPWCNLRYCTDPSTVEDKCEKACRPEEE | |
| | CLALNSTWGCFCRQDLNSSDPLCDELCRTVIAAAVLESFIVSVLLSAFC | |
| | IHCYHKFAHKPPISSAEMTERRPAQAFPVSYSSSGARRPSLDSMENQVS | |
| | VDAFKILEDPKWEFPRKNLVLGKTLGEGEFGKVVKATAFHLKGRAGYTT | |
| | VAVKMLKENASPSELRDLLSEFNVLKQVNHPHVIKLYGACSQDGPLLLI | |
| | VEYAKYGSLRGELRESRKVGPGYLGSGGSRNSSSLDHPDERALTMGDLI | |
| | SFAWQISQGMQYLAEMKLVHRDLAARNILVAEGRKMKISDEGLSRDVYE | |
| | EDSYVKRSQGRIPVKWMAIESLFDHIYTTQSDVWSFGVLLWEIVTLGGN | |
| | PYPGIPPERLFNLLKTGHRMERPDNCSEEMYRLMLQCWKQEPDKRPVFA | |
| | DISKDLEKMMVKRRDYLDLAASTPSDSLIYDDGLSEEETPLVDCNNAPL | |
| | PRALPSTWIENKLYGRISHAFTRF | |
| PPP2R5A- | MSSSSPPAGAASAAISASEKVDGFTRKSVRKAQRQKRSQGSSQFRSQGS | 70 |
| RET | QAELHPLPQLKGMSDPNWPGESPVPLTRADGTNTGFPRYPNDSVYANWM | |
| | LSPSAAKLMDTFDS | |
| GRM7-RET | MVQLRKLLRVLTLMKFPCCVLEVLLCALAAAARGQEMYAPHSIRIEGDV | 72 |
| | TLGGLFPVHAKGPSGVPCGDIKRENGIHRLEAMLYALDQINSDPNLLPN | |
| | VTLGARILDTCSRDTYALEQSLTFVQALIQKDTSDVRCTNGEPPVFVKP | |
| | EKVVGVIGASGSSVSIMVANILRLFQIPQISYASTAPELSDDRRYDFFS | |
| | RVVPPDSFQAQAMVDIVKALGWNYVSTLASEGSYGEKGVESFTQISKEA | |
| | GGLCIAQSVRIPQERKDRTIDEDRIIKQLLDTPNSRAVVIFANDEDIKQ | |
| | ILAAAKRADQVGHFLWVGSDSWGSKINPLHQHEDIAEGAITIQPKRATV | |
| | EGFDAYFTSRTLENNRRNVWFAEYWEENENCKLTISGSKKEDTDRKCTG | |
| | QERIGKDSNYEQEGKVQFVIDAVYAMAHALHHMNKDLCADYRGVCPEME | |
| | QAGGKKLLKYIRNVNFNGSAGTPVMENKNGDAPGRYDIFQYQTTNTSNP | |
| | GYRLIGQWTDELQLNIEDMQWGKGVREIPASVCTLPCKPGQRKKTQKGT | |
| | PCCWTCEPCDGYQYQFDEMTCQHCPYDQRPNENRTGCQDIPIIKLEWHS | |
| | PWAVIPVELAMLGIIATIFVMATFIRYNDTPIVRASGRELSYVLLTGIF | |
| | LCYIITELMIAKPDVAVCSFRRVELGLGMCISYAALLTKTNRIYRIFEQ | |
| | GKKSVTAPRLISPTSQLAITSSLISVQLLGVFIWEGVDPPNIIIDYDEH | |
| | KTMNPEQARGVLKCDITDLQIICSLGYSILLMVTCTVYAIKTRGVPENF | |
| | NEAKPIGFTMYTTCIVWLAFIPIFFGTAQSAEKLYIQTTTLTISMNLSA | |
| | SVALGMLYMPKVYIIIFHPELNVQKRKRSFKAVVTAATMSSRLSHKPSD | |
| | RPNGEAKTELCENVDPNNRGFPLLTVYLKVFLSPTSLREGECQWPGCAR | |
| | VYFSFENTSFPACSSLKPRELCFPETRPSFRIRENRPPGTFHQFRLLPV | |
| | QFLCPNISVAYRLLEGEGLPFRCAPDSLEVSTRWALDREQREKYELVAV | |
| | CTVHAGAREEVVMVPFPVTVYDEDDSAPTEPAGVDTASAVVEFKRKEDT | |
| | VVATLRVEDADVVPASGELVRRYTSTLLPGDTWAQQTERVEHWPNETSV | |
| | QANGSFVRATVHDYRLVLNRNLSISENRTMQLAVLVNDSDFQGPGAGVL | |
| | LLHFNVSVLPVSLHLPSTYSLSVSRRARRFAQIGKVCVENCQAFSGINV | |
| | QYKLHSSGANCSTLGVVTSAEDTSGILFVNDTKALRRPKCAELHYMVVA | |
| | TDQQTSRQAQAQLLVTVEGSYVAEEAGCPLSCAVSKRRLECEECGGLGS | |
| | PTGRCEWRQGDGKGITRNFSTCSPSTKTCPDGHCDVVETQDINICPQDC | |
| | LRGSIVGGHEPGEPRGIKAGYGTCNCFPEEEKCFCEPEDIQDPLCDELC | |
| | RTVIAAAVLFSFIVSVLLSAFCIHCYHKFAHKPPISSAEMTERRPAQAF | |
| | PVSYSSSGARRPSLDSMENQVSVDAFKILEDPKWEFPRKNLVLGKTLGE | |
| | GEFGKVVKATAFHLKGRAGYTTVAVKMLKENASPSELRDLLSEENVLKQ | |
| | VNHPHVIKLYGACSQDGPLLLIVEYAKYGSLRGFLRESRKVGPGYLGSG | |
| | GSRNSSSLDHPDERALTMGDLISFAWQISQGMQYLAEMKLVHRDLAARN | |
| | ILVAEGRKMKISDFGLSRDVYEEDSYVKRSQGRIPVKWMAIESLEDHIY | |
| | TTQSDVWSFGVLLWEIVTLGGNPYPGIPPERLENLLKTGHRMERPDNCS | |
| | EEMYRLMLQCWKQEPDKRPVFADISKDLEKMMVKRRDYLDLAASTPSDS | |
| | LIYDDGLSEEETPLVDCNNAPLPRALPSTWIENKLYGRISHAFTRE | |
| MYH14-RET | MAAVTMSVPGRKAPPRPGPVPEAAQPFLFTPRGPSAGGGPGSGTSPQVE | 74 |
| | WTARRLVWVPSELHGFEAAALRDEGEEEAEVELAESGRRLRLPRDQIQR | |
| | MNPPKFSKAEDMAELTCLNEASVLHNLRERYYSGLIYTYSGLFCVVINP | |
| | YKQLPIYTEAIVEMYRGKKRHEVPPHVYAVTEGAYRSMLQDREDQSILC | |
| | TGESGAGKTENTKKVIQYLAHVASSPKGRKEPGVPGELERQLLQANPIL | |
| | EAFGNAKTVKNDNSSRFGKFIRINFDVAGYIVGANIETYLLEKSRAIRQ | |
| | AKDECSFHIFYQLLGGAGEQLKADLLLEPCSHYRFLINGPSSSPGQERE | |
| | LFQETLESLRVLGFSHEEIISMLRMVSAVLQFGNIALKRERNTDQATMP | |
| | DNTAAQKLCRLLGLGVTDESRALLTPRIKVGRDYVQKAQTKEQADFALE | |
| | ALAKATYERLERWLVLRLNRALDRSPRQGASFLGILDIAGFEIFQLNSF | |
| | EQLCINYTNEKLQQLENHTMFVLEQEEYQREGIPWTFLDFGLDLQPCID | |
| | LIERPANPPGLLALLDEECWFPKATDKSFVEKVAQEQGGHPKFQRPRHL | |
| | RDQADFSVLHYAGKVDYKANEWLMKNMDPLNDNVAALLHQSTDRLTAEI | |
| | WKDVEGIVGLEQVSSLGDGPPGGRPRRGMERTVGQLYKESLSRLMATLS | |
| | NTNPSFVRCIVPNHEKRAGKLEPRLVLDQLRCNGVLEGIRICRQGFPNR | |
| | ILFQEFRQRYEILTPNAIPKGFMDGKQACEKMIQALELDPNLYRVGQSK | |
| | IFFRAGVLAQLEEERDLKVTDIIVSFQAAARGYLARRAFQKRQQQQSAL | |
| | RVMQRNCAAYLKLRHWQWWRLFTKVKPLLQVTRQDEVLQARAQELQKVQ | |
| | ELQQQSAREVGELQGRVAQLEEERARLAEQLRAFAELCAEAEETRGRLA | |
| | ARKQELELVVSELEARVGEEEECSRQMQTEKKRLQQHIQELEAHLEAEE | |
| | GARQKLQLEKVTTEAKMKKFEEDLLLLEDQNSKLSKERKLLEDRLAEFS | |
| | SQAAEEEEKVKSLNKLRLKYEATIADMEDRLRKEEKGRQELEKLKRRLD | |
| | GESSELQEQMVEQQQRAEELRAQLGRKEEELQAALARAEDEGGARAQLL | |
| | KSLREAQAALAEAQEDLESERVARTKAEKQRRDLGEELEALRGELEDTL | |
| | DSTNAQQELRSKREQEVTELKKTLEEETRIHEAAVQELRQRHGQALGEL | |
| | AEQLEQARRGKGAWEKTRLALEAEVSELRAELSSLQTARQEGEQRRRRL | |
| | ELQLQEVQGRAGDGERARAEAAEKLQRAQAELENVSGALNEAESKTIRL | |
| | SKELSSTEAQLHDAQELLQEETRAKLALGSRVRAMEAEAAGLREQLEEE | |
| | AAARERAGRELQTAQAQLSEWRRRQEEEAGALEAGEEARRRAAREAEAL | |
| | TQRLAEKTETVDRLERGRRRLQQELDDATMDLEQQRQLVSTLEKKQRKE | |
| | DQLLAEEKAAVLRAVEERERAEAEGREREARALSLTRALEEEQEAREEL | |
| | ERQNRALRAELEALLSSKDDVGKSVHELERACRVAEQAANDLRAQVTEL | |
| | EDELTAAEDAKLRLEVTVQALKTQHERDLQGRDEAGEERRRQLAKQLRD | |
| | AEVERDEERKQRTLAVAARKKLEGELEELKAQMASAGQGKEEAVKQLRK | |
| | MQAQMKELWREVEETRTSREEIFSQNRESEKRLKGLEAEVLRLQEELAA | |
| | SDRARRQAQQDRDEMADEVANGNLSKAAILEEKRQLEGRLGQLEEELEE | |
| | EQSNSELLNDRYRKLLLQEDPKWEFPRKNLVLGKTLGEGEFGKVVKATA | |
| | FHLKGRAGYTTVAVKMLKENASPSELRDLLSEFNVLKQVNHPHVIKLYG | |
| | ACSQDGPLLLIVEYAKYGSLRGFLRESRKVGPGYLGSGGSRNSSSLDHP | |
| | DERALTMGDLISFAWQISQGMQYLAEMKLVHRDLAARNILVAEGRKMKI | |
| | SDFGLSRDVYEEDSYVKRSQGRIPVKWMAIESLEDHIYTTQSDVWSFGV | |
| | LLWEIVTLGGNPYPGIPPERLENLLKTGHRMERPDNCSEEMYRLMLQCW | |
| | KQEPDKRPVFADISKDLEKMMVKRRDYLDLAASTPSDSLIYDDGLSEEE | |
| | TPLVDCNNAPLPRALPSTWIENKLYGRISHAFTRE | |
| MGEA5-RET | MVQKESQATLEERESELSSNPAASAGASLEPPAAPAPGEDNPAGAGGAA | 75 |
| | VAGAAGGARRFLCGVVEGFYGRPWVMEQRKELFRRLQKWELNTYLYAPK | |
| | DDYKHRMFWREMYSVEEAEQLMTLISAAREYEIEFIYAISPGLDITESN | |
| | PKEVSTLKRKLDQVSQFGCRSFALLEDDIDHNMCAADKEVESSFAHAQV | |
| | SITNEIYQYLGEPETFLFCPTEYCGTFCYPNVSQSPYLRTVGEKLLPGI | |
| | EVLWTGPKVVSKEIPVESIEEVSKIIKRAPVIWDNIHANDYDQKRLFLG | |
| | PYKGRSTELIPRLKGVLTNPNCEFEANYVAIHTLATWYKSNMNGVRKDV | |
| | VMTDSEDSTVSIQIKLENEGSDEDIETDVLYSPQMALKLALTEWLQEFG | |
| | VPHQYSSRQVAHSGAKASVVDGTPLVAAPSLNATTVVTTVYQEPIMSQG | |
| | AALSGEPTTLTKEEEKKQPDEEPMDMVVEKQEETDHKNDNQILSEIVEA | |
| | KMAEELKPMDTDKESIAESKSPEMSMQEDCISDIAPMQTDEQTNKEQFV | |
| | PGPNEKPLYTAEPVTLEDLQLLADLFYLPYEHGPKGAQMLREFQWLRAN | |
| | SSVVSVNCKGKDSEKIEEWRSRAAKFEEMCGLVMGMFTRLSNCANRTIL | |
| | YDMYSYVWDIKSIMSMVKSFVQWLGCRSHSSAQFLIGDQEPWAFRGGLA | |
| | GEFQRLLPIDGANDLFFQPPPLTPTSKVYTIRPYFPKDEEDPKWEFPRK | |
| | NLVLGKTLGEGEFGKVVKATAFHLKGRAGYTTVAVKMLKENASPSELRD | |
| | LLSEFNVLKQVNHPHVIKLYGACSQDGPLLLIVEYAKYGSLRGELRESR | |
| | KVGPGYLGSGGSRNSSSLDHPDERALTMGDLISFAWQISQGMQYLAEMK | |
| | LVHRDLAARNILVAEGRKMKISDEGLSRDVYEEDSYVKRSQGRIPVKWM | |
| | AIESLFDHIYTTQSDVWSFGVLLWEIVTLGGNPYPGIPPERLENLLKTG | |
| | HRMERPDNCSEEMYRLMLQCWKQEPDKRPVFADISKDLEKMMVKRRDYL | |
| | DLAASTPSDSLIYDDGLSEEETPLVDCNNAPLPRALPSTWIENKLYGRI | |
| | SHAFTRE | |
| SAMD4A- | MMFRDQVGVLAGWFKGWNECEQTVALLSLLKRVSQTQARFLQLCLEHSL | 76 |
| RET | ADCAELHVLEREANSPDPLCDELCRTVIAAAVLFSFIVSVLLSAFCIHC | |
| | YHKFAHKPPISSAEMTERRPAQAFPVSYSSSGARRPSLDSMENQVSVDA | |
| | FKILEDPKWEFPRKNLVLGKTLGEGEFGKVVKATAFHLKGRAGYTTVAV | |
| | KMLKENASPSELRDLLSEFNVLKQVNHPHVIKLYGACSQDGPLLLIVEY | |
| | AKYGSLRGFLRESRKVGPGYLGSGGSRNSSSLDHPDERALTMGDLISFA | |
| | WQISQGMQYLAEMKLVHRDLAARNILVAEGRKMKISDFGLSRDVYEEDS | |
| | YVKRSQGRIPVKWMAIESLFDHIYTTQSDVWSFGVLLWEIVTLGGNPYP | |
| | GIPPERLENLLKTGHRMERPDNCSEEMYRLMLQCWKQEPDKRPVFADIS | |
| | KDLEKMMVKRRDYLDLAASTPSDSLIYDDGLSEEETPLVDCNNAPLPRA | |
| | LPSTWIENKLYGRISHAFTRE | |
| ACPP-RET | MRAAPLLLARAASLSLGFLFLLFFWLDRSVLAKELKFVTLVFRHGDRSP | 77 |
| | IDTFPTDPIKESSWPQGFGQLTQLGMEQHYELGEYIRKRYRKELNESYK | |
| | HEQVYIRSTDVDRTLMSAMTNLAALFPPEGVSIWNPILLWQPIPVHTVP | |
| | LSEDQLLYLPFRNCPRFQELESETLKSEEFQKRLHPYKDFIATLGKLSG | |
| | LHGQDLFGIWSKVYDPLYCESVHNFTLPSWATEDTMTKLRELSELSLLS | |
| | LYGIHKQKEKSRLQGGVLVNEILNHMKRATQIPSYKKLIMYSAHDTTVS | |
| | GLQMALDVYNGLLPPYASCHLTELYFEKGEYFVEMYYRNETQHEPYPLM | |
| | LPGCSPSCPLERFAELVGPVIPQDWSTECMTTNSHQDPLCDELCRTVIA | |
| | AAVLFSFIVSVLLSAFCIHCYHKFAHKPPISSAEMTERRPAQAFPVSYS | |
| | SSGARRPSLDSMENQVSVDAFKILEDPKWEFPRKNLVLGKTLGEGEFGK | |
| | VVKATAFHLKGRAGYTTVAVKMLKENASPSELRDLLSEENVLKQVNHPH | |
| | VIKLYGACSQDGPLLLIVEYAKYGSLRGFLRESRKVGPGYLGSGGSRNS | |
| | SSLDHPDERALTMGDLISFAWQISQGMQYLAEMKLVHRDLAARNILVAE | |
| | GRKMKISDFGLSRDVYEEDSYVKRSQGRIPVKWMAIESLFDHIYTTQSD | |
| | VWSFGVLLWEIVTLGGNPYPGIPPERLENLLKTGHRMERPDNCSEEMYR | |
| | LMLQCWKQEPDKRPVFADISKDLEKMMVKRRDYLDLAASTPSDSLIYDD | |
| | GLSEEETPLVDCNNAPLPRALPSTWIENKLYGRISHAFTRE | |
| BMS1-RET | MEAKDQKKHRKKNSGPKAAKKKKRLLQDLQLGDEEDARKRNPKAFAVQS | 78 |
| | AVRMARSFHRTQDLKTKKHHIPVVDRTPLEPPPIVVVVMGPPKVGKSTL | |
| | IQCLIRNFTRQKLTEIRGPVTIVSGKKRRLTIIECGCDINMMIDLAKVA | |
| | DLVLMLIDASFGFEMETFEFLNICQVHGFPKIMGVLTHLDSFKHNKQLK | |
| | KTKKRLKHRFWTEVYPGAKLFYLSGMVHGEYQNQEIHNLGRFITVMKER | |
| | PLTWQTSHPYILADRMEDLTNPEDIRTNIKCDRKVSLYGYLRGAHLKNK | |
| | SQIHMPGVGDFAVSDISFLPDPCALPEQQKKRCLNEKEKLVYAPLSGVG | |
| | GVLYDKDAVYVDLGGSHVFQDEVGPTHELVQSLISTHSTIDAKMASSRV | |
| | TLFSDSKPLGSEDIDNQGLMMPKEEKQMDLNTGRMRRKAIFGDEDESGD | |
| | SDDEEDDEMSEDDGLENGSSDEEAEEEENAEMTDQYMAVKGIKRRKLEL | |
| | EEDSEMDLPAFADSDDDLERSSAEEGEAEEADESSEEEDCTAGEKGISG | |
| | SKAAGEGSKAGLSPANCQSDRVNLEKSLLMKKAALPTEDSGHCTAEEVE | |
| | ASEDESEESSSLSAEEEDSENEEAIRKKLSKPSQVSSGQKLGPQNFIDE | |
| | TSDIENLLKEEEDYKEENNDSKETSGALKWKEDLSRKAAEAFLRQQQAA | |
| | PNLRKLIYGTVTEDNEEEDDDTLEELGGLERVNQPDRECKHKADSLDCS | |
| | RFLVEAPHDWDLEEVMNSIRDCFVTGKWEDDKDAAKVLAEDGGSIVGGH | |
| | EPGEPRGIKAGYGTCNCFPEEEKCFCEPEDIQDPLCDELCRTVIAAAVL | |
| | FSFIVSVLLSAFCIHCYHKFAHKPPISSAEMTERRPAQAFPVSYSSSGA | |
| | RRPSLDSMENQVSVDAFKILEDPKWEFPRKNLVLGKTLGEGEFGKVVKA | |
| | TAFHLKGRAGYTTVAVKMLKENASPSELRDLLSEFNVLKQVNHPHVIKL | |
| | YGACSQDGPLLLIVEYAKYGSLRGELRESRKVGPGYLGSGGSRNSSSLD | |
| | HPDERALTMGDLISFAWQISQGMQYLAEMKLVHRDLAARNILVAEGRKM | |
| | KISDFGLSRDVYEEDSYVKRSQGRIPVKWMAIESLEDHIYTTQSDVWSF | |
| | GVLLWEIVTLGGNPYPGIPPERLENLLKTGHRMERPDNCSEEMYRLMLQ | |
| | CWKQEPDKRPVFADISKDLEKMMVKRRDYLDLAASTPSDSLIYDDGLSE | |
| | EETPLVDCNNAPLPRALPSTWIENKLYGRISHAFTRE | |
| CEP135-RET | MTTAVERKYINIRKRLDQLGYRQTLTVECLPLVEKLESDLVHTTESLRQ | 79 |
| | SKLSAVKAEKESANFDFVLEPYKLENARLSRENNELYLELMKLREHSDQ | |
| | HVKELKTSLKKCARETADLKELNNQYAHKLKLLEKESKAKNERIQQLQE | |
| | KNLHAVVQTPGGKKRSIAFRRQRMQIDEPVPPSEVSSYPVPQPDDPYIA | |
| | DLLQVADNRIQELQQEVHQLQEKLAMMESGVRDYSKQIELREREIERLS | |
| | VALDGGRSPDVLSLESRNKTNEKLIAHLNIQVDELQQANKDLEKRIREL | |
| | METKETVTSEVVNLSNKNEKLCQELTEIDQLAQQLERHKEEVLETADKE | |
| | LGEAKKEIKRKLSEMQDLEETMAKLQLELNLCQKEKERLSDELLVKSDL | |
| | ETVVHQLEQEKQRLSKKVESFAVTERQLTLEVERMRLEHGIKRRDRSPS | |
| | RLDTFLKGIEEERDYYKKELERLQHIIQRRSCSTSYSAREKSSIFRTPE | |
| | KEDPKWEFPRKNLVLGKTLGEGEFGKVVKATAFHLKGRAGYTTVAVKML | |
| | KENASPSELRDLLSEFNVLKQVNHPHVIKLYGACSQDGPLLLIVEYAKY | |
| | GSLRGFLRESRKVGPGYLGSGGSRNSSSLDHPDERALTMGDLISFAWQI | |
| | SQGMQYLAEMKLVHRDLAARNILVAEGRKMKISDEGLSRDVYEEDSYVK | |
| | RSQGRIPVKWMAIESLEDHIYTTQSDVWSFGVLLWEIVTLGGNPYPGIP | |
| | PERLFNLLKTGHRMERPDNCSEEMYRLMLQCWKQEPDKRPVFADISKDL | |
| | EKMMVKRRDYLDLAASTPSDSLIYDDGLSEEETPLVDCNNAPLPRALPS | |
| | TWIENKLYGRISHAFTRE | |
| EEA1-RET | MLRRILQRTPGRVGSQGSDLDSSATPINTVDVNNESSSEGFICPQCMKS | 80 |
| | LGSADELFKHYEAVHDAGNDSGHGGESNLALKRDDVTLLRQEVQDLQAS | |
| | LKEEKWYSEELKKELEKYQGLQQQEAKPDGLVTDSSAELQSLEQQLEEA | |
| | QTENFNIKQMKDLFEQKAAQLATEIADIKSKYDEERSLREAAEQKVTRL | |
| | TEELNKEATVIQDLKTELLQRPGIEDVAVLKKELVQVQTLMDNMTLERE | |
| | RESEKLKDECKKLQSQYASSEATISQLRSELAKGPQEVAVYVQELQKLK | |
| | SSVNELTQKNQTLTENLLKKEQDYTKLEEKHNEESVSKKNIQATLHQKD | |
| | LDCQQLQSRLSASETSLHRIHVELSEKGEATQKLKEELSEVETKYQHLK | |
| | AEFKQLQQQREEKEQHGLQLQSEINQLHSKLLETERQLGEAHGRLKEQR | |
| | QLSSEKLMDKEQQVADLQLKLSRLEEQLKEKVINSTELQHQLDKTKQQH | |
| | QEQQALQQSTTAKLREAQEDPKWEFPRKNLVLGKTLGEGEFGKVVKATA | |
| | FHLKGRAGYTTVAVKMLKENASPSELRDLLSEFNVLKQVNHPHVIKLYG | |
| | ACSQDGPLLLIVEYAKYGSLRGELRESRKVGPGYLGSGGSRNSSSLDHP | |
| | DERALTMGDLISFAWQISQGMQYLAEMKLVHRDLAARNILVAEGRKMKI | |
| | SDFGLSRDVYEEDSYVKRSQGRIPVKWMAIESLEDHIYTTQSDVWSFGV | |
| | LLWEIVTLGGNPYPGIPPERLENLLKTGHRMERPDNCSEEMYRLMLQCW | |
| | KQEPDKRPVFADISKDLEKMMVKRRDYLDLAASTPSDSLIYDDGLSEEE | |
| | TPLVDCNNAPLPRALPSTWIENKLYGRISHAFTRE | |
| CSGALNACT2- | MPRRGLILHTRTHWLLLGLALLCSLVLFMYLLECAPQTDGNASLPGVVG | 81 |
| RET | ENYGKEYYQALLQEQEEHYQTRATSLKRQIAQLKQELQEMSEKMRSLQE | |
| | RRNVGANGIGYQSNKEQAPSDLLEFLHSQIDKAEVSIGAKLPSEYGVIP | |
| | FESFTLMKVFQLEMGLTRHPEEKPVRKDKRDELVEVIEAGLEVINNPDE | |
| | DDEQEDEEGPLGEKLIFNENDEVEGYYRTERDKGTQYELFFKKADLTEY | |
| | RHVTLFRPFGPLMKVKSEMIDITRSIINIIVPLAERTEAFVQFMQNERD | |
| | VCIHQDKKIHLTVVYFGKEGLSKVKSILESVTSESNFHNYTLVSLNEEF | |
| | NRGRGLNVGARAWDKGEVLMFFCDVDIYFSAEFLNSCRLNAEPGKKVFY | |
| | PVVFSLYNPAIVYANQEVPPPVEQQLEDPKWEFPRKNLVLGKTLGEGEF | |
| | GKVVKATAFHLKGRAGYTTVAVKMLKENASPSELRDLLSEENVLKQVNH | |
| | PHVIKLYGACSQDGPLLLIVEYAKYGSLRGELRESRKVGPGYLGSGGSR | |
| | NSSSLDHPDERALTMGDLISFAWQISQGMQYLAEMKLVHRDLAARNILV | |
| | AEGRKMKISDFGLSRDVYEEDSYVKRSQGRIPVKWMAIESLEDHIYTTQ | |
| | SDVWSFGVLLWEIVTLGGNPYPGIPPERLENLLKTGHRMERPDNCSEEM | |
| | YRLMLQCWKQEPDKRPVFADISKDLEKMMVKRRDYLDLAASTPSDSLIY | |
| | DDGLSEEETPLVDCNNAPLPRALPSTWIENKLYGRISHAFTRF | |
| KIAA1217- | MEENESQKCEPCLPYSADRRQMQEQGKGNLHVTSPEDAECRRTKERLSN | 82 |
| RET | GNSRGSVSKSSRNIPRRHTLGGPRSSKEILGMQTSEMDRKREAFLEHLK | |
| | QKYPHHASAIMGHQERLRDQTRSPKLSHSPQPPSLGDPVEHLSETSADS | |
| | LEAMSEGDAPTPESRGSRTRASLPVVRSTNQTKERSLGVLYLQYGDETK | |
| | QLRMPNEITSADTIRALFVSAFPQQLTMKMLESPSVAIYIKDESRNVYY | |
| | ELNDVRNIQDRSLLKVYNKDPAHAFNHTPKTMNGDMRMQRELVYARGDG | |
| | PGAPRPGSTAHPPHAIPNSPPSTPVPHSMPPSPSRIPYGGTRSMVVPGN | |
| | ATIPRDRISSLPVSRPISPSPSAILERRDVKPDEDMSGKNIAMYRNEGE | |
| | YADPYLYHEGRMSIASSHGGHPLDVPDHIIAYHRTAIRSASAYCNPSMQ | |
| | AEMHMEQSLYRQKSRKYPDSHLPTLGSKTPPASPHRVSDLRMIDMHAHY | |
| | NAHGPPHTMQPDRASPSRQAFKKEPGTLVYIEKPRSAAGLSSLVDLGPP | |
| | LMEKQVFAYSTATIPKDRETRERMQAMEKQIASLTGLVQSALEKGPITS | |
| | YSKDASSEKMMKTTANRNHTDSAGTPHVSGGKMLSALESTVPPSQPPPV | |
| | GTSAIHMSLLEMRRSVAELRLQLQQMRQLQLQNQELLRAMMKKAELEIS | |
| | GKVMETMKRLEDPVQRQRVLVEQERQKYLHEEEKIVKKLCELEDEVEDL | |
| | KKDSTAASRLVTLKDVEDGAFLLRQVGEAVATLKDVAEEAGCPLSCAVS | |
| | KRRLECEECGGLGSPTGRCEWRQGDGKGITRNESTCSPSTKTCPDGHCD | |
| | VVETQDINICPQDCLRGSIVGGHEPGEPRGIKAGYGTCNCFPEEEKCFC | |
| | EPEDIQDPLCDELCRTVIAAAVLESFIVSVLLSAFCIHCYHKFAHKPPI | |
| | SSAEMTERRPAQAFPVSYSSSGARRPSLDSMENQVSVDAFKILEDPKWE | |
| | FPRKNLVLGKTLGEGEFGKVVKATAFHLKGRAGYTTVAVKMLKENASPS | |
| | ELRDLLSEFNVLKQVNHPHVIKLYGACSQDGPLLLIVEYAKYGSLRGFL | |
| | RESRKVGPGYLGSGGSRNSSSLDHPDERALTMGDLISFAWQISQGMQYL | |
| | AEMKLVHRDLAARNILVAEGRKMKISDFGLSRDVYEEDSYVKRSQGRIP | |
| | VKWMAIESLFDHIYTTQSDVWSFGVLLWEIVTLGGNPYPGIPPERLENL | |
| | LKTGHRMERPDNCSEEMYRLMLQCWKQEPDKRPVFADISKDLEKMMVKR | |
| | RDYLDLAASTPSDSLIYDDGLSEEETPLVDCNNAPLPRALPSTWIENKL | |
| | YGRISHAFTRF | |
| SORBS1-RET | MSSECDGGSKAVMNGLAPGSNGQDKDMDLTKICTGKGAVTLRASSSYRE | 83 |
| | TPSSSPASPQETRQHESKPDEWRLSSSADANGNAQPSSLAAKGYRSVHP | |
| | NLPSDKSQDSSPLLNEVSSSLIGTDSQAFPSVSKPSSAYPSTTIVNPTI | |
| | VLLQHNREQQKRLSSLSDPVSERRVGEQDSAPTQEKPTSPGKAIEKRAK | |
| | DDSRRVVKSTQDLSDVSMDEVGIPLRNTERSKDWYKTMFKQIHKLNRDD | |
| | DSDLYSPRYSFSEDTKSPLSVPRSKSEMSYIDGEKVVKRSATLPLPARS | |
| | SSLKSSSERNDWEPPDKKVDTRKYRAEPKSIYEYQPGKSSVLTNEKMSS | |
| | AISPTPEISSETPGYIYSSNFHAVKRESDGAPGDLTSLENERQIYKSVL | |
| | EGGDIPLQGLSGLKRPSSSASTKDSESPRHFIPADYLESTEEFIRRRHD | |
| | DKEMRPARAKFDFKAQTLKELPLQKGDIVYIYKQIDQNWYEGEHHGRVG | |
| | IFPRTYIELLPPAEKAQPKKLTPVQVLEYGEAIAKENENGDTQVEMSER | |
| | KGERITLLRQVDENWYEGRIPGTSRQGIFPITYVDVIKRPLVKNPVDYM | |
| | DLPFSSSPSRSATASPQIGKVCVENCQAFSGINVQYKLHSSGANCSTLG | |
| | VVTSAEDTSGILFVNDTKALRRPKCAELHYMVVATDQQTSRQAQAQLLV | |
| | TVEGSYVAEEAGCPLSCAVSKRRLECEECGGLGSPTGRCEWRQGDGKGI | |
| | TRNFSTCSPSTKTCPDGHCDVVETQDINICPQDCLRGSIVGGHEPGEPR | |
| | GIKAGYGTCNCFPEEEKCFCEPEDIQDPLCDELCRTVIAAAVLESFIVS | |
| | VLLSAFCIHCYHKFAHKPPISSAEMTERRPAQAFPVSYSSSGARRPSLD | |
| | SMENQVSVDAFKILEDPKWEFPRKNLVLGKTLGEGEFGKVVKATAFHLK | |
| | GRAGYTTVAVKMLKENASPSELRDLLSEFNVLKQVNHPHVIKLYGACSQ | |
| | DGPLLLIVEYAKYGSLRGFLRESRKVGPGYLGSGGSRNSSSLDHPDERA | |
| | LTMGDLISFAWQISQGMQYLAEMKLVHRDLAARNILVAEGRKMKISDFG | |
| | LSRDVYEEDSYVKRSQGRIPVKWMAIESLEDHIYTTQSDVWSFGVLLWE | |
| | IVTLGGNPYPGIPPERLFNLLKTGHRMERPDNCSEEMYRLMLQCWKQEP | |
| | DKRPVFADISKDLEKMMVKRRDYLDLAASTPSDSLIYDDGLSEEETPLV | |
| | DCNNAPLPRALPSTWIENKLYGRISHAFTRE | |
| SORBS1-RET | MSSECDGGSKAVMNGLAPGSNGQDKDMDLTKICTGKGAVTLRASSSYRE | 84 |
| | TPSSSPASPQETRQHESKPDEWRLSSSADANGNAQPSSLAAKGYRSVHP | |
| | NLPSDKSQDSSPLLNEVSSSLIGTDSQAFPSVSKPSSAYPSTTIVNPTI | |
| | VLLQHNREQQKRLSSLSDPVSERRVGEQDSAPTQEKPTSPGKAIEKRAK | |
| | DDSRRVVKSTQDLSDVSMDEVGIPLRNTERSKDWYKTMFKQIHKLNRDD | |
| | DSDLYSPRYSFSEDTKSPLSVPRSKSEMSYIDGEKVVKRSATLPLPARS | |
| | SSLKSSSERNDWEPPDKKVDTRKYRAEPKSIYEYQPGKSSVLTNEKMSS | |
| | AISPTPEISSETPGYIYSSNFHAVKRESDGAPGDLTSLENERQIYKSVL | |
| | EGGDIPLQGLSGLKRPSSSASTKDSESPRHFIPADYLESTEEFIRRRHD | |
| | DKEMRPARAKFDFKAQTLKELPLQKGDIVYIYKQIDQNWYEGEHHGRVG | |
| | IFPRTYIELLPPAEKAQPKKLTPVQVLEYGEAIAKENENGDTQVEMSER | |
| | KGERITLLRQVDENWYEGRIPGTSRQGIFPITYVDVIKRPLVKNPVDYM | |
| | DLPESSSPSRSATASPQQPQAQQRRVTPDRSQTSQDLESYQALYSYIPQ | |
| | NDDELELRDGDIVDVMEKCDDGWFVDPLCDELCRTVIAAAVLFSFIVSV | |
| | LLSAFCIHCYHKFAHKPPISSAEMTERRPAQAFPVSYSSSGARRPSLDS | |
| | MENQVSVDAFKILEDPKWEFPRKNLVLGKTLGEGEFGKVVKATAFHLKG | |
| | RAGYTTVAVKMLKENASPSELRDLLSEFNVLKQVNHPHVIKLYGACSQD | |
| | GPLLLIVEYAKYGSLRGFLRESRKVGPGYLGSGGSRNSSSLDHPDERAL | |
| | TMGDLISFAWQISQGMQYLAEMKLVHRDLAARNILVAEGRKMKISDEGL | |
| | SRDVYEEDSYVKRSQGRIPVKWMAIESLFDHIYTTQSDVWSFGVLLWEI | |
| | VTLGGNPYPGIPPERLENLLKTGHRMERPDNCSEEMYRLMLQCWKQEPD | |
| | KRPVFADISKDLEKMMVKRRDYLDLAASTPSDSLIYDDGLSEEETPLVD | |
| | CNNAPLPRALPSTWIENKLYGRISHAFTRE | |
| MPRIP-RET | MSAAKENPCRKFQANIFNKSKCQNCFKPRESHLLNDEDLTQAKPIYGGW | 85 |
| | LLLAPDGTDEDNPVHRSRKWQRRFFILYEHGLLRYALDEMPTTLPQGTI | |
| | NMNQCTDVVDGEGRTGQKFSLCILTPEKEHFIRAETKEIVSGWLEMLMV | |
| | YPRTNKQNQKKKRKVEPPTPQEPGPAKVAVTSSSSSSSSSSSIPSAEKV | |
| | PTTKSTLWQEEMRTKDQPDGSSLSPAQSPSQSQPPAASSLREPGLESKE | |
| | EESAMSSDRMDCGRKVRVESGYFSLEKTKQDLKAEEQQLPPPLSPPSPS | |
| | TPNHRRSQVIEKFEALDIEKAEHMETNAVGPSPSSDTRQGRSEKRAFPR | |
| | KRDFTNEAPPAPLPDASASPLSPHRRAKSLDRRSTEPSVTPDLLNEKKG | |
| | WLTKQYEDGQWKKHWFVLADQSLRYYRDSVAEEAADLDGEIDLSACYDV | |
| | TEYPVQRNYGFQIHTKEGEFTLSAMTSGIRRNWIQTIMKHVHPTTAPDV | |
| | TSSLPEEKNKSSCSFETCPRPTEKQEAELGEPDPEQKRSRARERRREGR | |
| | SKTEDWAEFRPIQQALAQERVGGVGPADTHEPLRPEAEPGELERERARR | |
| | REERRKRFGMLDATDGPGTEDAALRMEVDRSPGLPMSDLKTHNVHVEIE | |
| | QRWHQVETTPLREEKQVPIAPVHLSSEDGGDRLSTHELTSLLEKELEQS | |
| | QKEASDLLEQNRLLQDQLRVALGREQSAREGYVLQATCERGFAAMEETH | |
| | QKKIEDLQRQHQRELEKLREEKDRLLAEETAATISAIEAMKNAHREEME | |
| | RELEKSQRSQISSVNSDVEALRRQYLEELQSVQRELEVLSEQYSQKCLE | |
| | NAHLAQALEAERQALRQCQRENQELNAHNQELNNRLAAEITRLRTLLTG | |
| | DGGGEATGSPLAQGKDAYELEVLLRVKESEIQYLKQEISSLKDELQTAL | |
| | RDKKYASDKYKDIYTELSIAKAKADCDISRLKEQLKAATEALGEKSPDS | |
| | ATVSGYDIMKSKSNPDFLKKDRSCVTRQLRNIRSKEDPKWEFPRKNLVL | |
| | GKTLGEGEFGKVVKATAFHLKGRAGYTTVAVKMLKENASPSELRDLLSE | |
| | FNVLKQVNHPHVIKLYGACSQDGPLLLIVEYAKYGSLRGFLRESRKVGP | |
| | GYLGSGGSRNSSSLDHPDERALTMGDLISFAWQISQGMQYLAEMKLVHR | |
| | DLAARNILVAEGRKMKISDFGLSRDVYEEDSYVKRSQGRIPVKWMAIES | |
| | LFDHIYTTQSDVWSFGVLLWEIVTLGGNPYPGIPPERLENLLKTGHRME | |
| | RPDNCSEEMYRLMLQCWKQEPDKRPVFADISKDLEKMMVKRRDYLDLAA | |
| | STPSDSLIYDDGLSEEETPLVDCNNAPLPRALPSTWIENKLYGRISHAF | |
| | TRF | |
| TFG-RET | MNGQLDLSGKLIIKAQLGEDIRRIPIHNEDITYDELVLMMQRVERGKLL | 86 |
| | SNDEVTIKYKDEDGDLITIFDSSDLSFAIQCSRILKLTLFVNGQPRPLE | |
| | SSQVKYLRRELIELRNKVNRLLDSLEPPGEPGPSTNIPENDTVDGREEK | |
| | SASDSSGKQSTQVMAASMSAFDPLKNQDEINKNVMSAFGLTDDQVSDPL | |
| | CDELCRTVIAAAVLESFIVSVLLSAFCIHCYHKFAHKPPISSAEMTERR | |
| | PAQAFPVSYSSSGARRPSLDSMENQVSVDAFKILEDPKWEFPRKNLVLG | |
| | KTLGEGEFGKVVKATAFHLKGRAGYTTVAVKMLKENASPSELRDLLSEE | |
| | NVLKQVNHPHVIKLYGACSQDGPLLLIVEYAKYGSLRGFLRESRKVGPG | |
| | YLGSGGSRNSSSLDHPDERALTMGDLISFAWQISQGMQYLAEMKLVHRD | |
| | LAARNILVAEGRKMKISDEGLSRDVYEEDSYVKRSQGRIPVKWMAIESL | |
| | FDHIYTTQSDVWSFGVLLWEIVTLGGNPYPGIPPERLENLLKTGHRMER | |
| | PDNCSEEMYRLMLQCWKQEPDKRPVFADISKDLEKMMVKRRDYLDLAAS | |
| | TPSDSLIYDDGLSEEETPLVDCNNAPLPRALPSTWIENKLYGRISHAFT | |
| | RF | |
| SPECC1L- | MKKASRSVGSVPKVSAISKTQTAEKIKPENSSSASTGGKLVKPGTAASL | 87 |
| RET | SKTKSSDDLLAGMAGGVTVTNGVKGKKSTCPSAAPSASAPAMTTVENKS | |
| | KISTGTASSTKRSTSTGNKESSSTRERLRERTRLNQSKKLPSAGQGAND | |
| | MALAKRSRSRTATECDVRMSKSKSDNQISDRAALEAKVKDLLTLAKTKD | |
| | VEILHLRNELRDMRAQLGINEDHSEGDEKSEKETIMAHQPTDVESTLLQ | |
| | LQEQNTAIREELNQLKNENRMLKDRLNALGFSLEQRLDNSEKLFGYQSL | |
| | SPEITPGNQSDGGGTLTSSVEGSAPGSVEDLLSQDENTLMDHQHSNSMD | |
| | NLDSECSEVYQPLTSSDDALDAPSSSESEGIPSIERSRKGSSGNASEVS | |
| | VACLTERIHQMEENQHSTSEELQATLQELADLQQITQELNSENERLGEE | |
| | KVILMESLCQQSDKLEHFSRQIEYFRSLLDEHHISYVIDEDVKSGRYME | |
| | LEQRYMDLAENARFEREQLLGVQQHLSNTLKMAEQDNKEAQEMIGALKE | |
| | RSHHMERIIESEQKGKAALAATLEEYKATVASDQIEMNRLKAQLENEKQ | |
| | KVAELYSIHNSGDKSDIQDLLESVRLDKEKAETLASSLQEDLAHTRNDA | |
| | NRLQDAIAKVEDEYRAFQEEAKKQIEDLNMTLEKLRSDLDEKETERSDM | |
| | KETIFELEDEVEQHRAVKLHDNLIISDLENTVKKLQDQKHDMEREIKTL | |
| | HRRLREESAEWRQFQADLQTAVVIANDIKSEAQEEIGDLKRRLHEAQEK | |
| | NEKLTKELEEIKSRKQEEERGRVYNYMNAVERDLAALRQGMGLSRRSST | |
| | SSEPTPTVKTLIKSFDSASQVPNPAAAAIPRTPLSPSPMKTPPAAAVSP | |
| | MQEDPKWEFPRKNLVLGKTLGEGEFGKVVKATAFHLKGRAGYTTVAVKM | |
| | LKENASPSELRDLLSEFNVLKQVNHPHVIKLYGACSQDGPLLLIVEYAK | |
| | YGSLRGFLRESRKVGPGYLGSGGSRNSSSLDHPDERALTMGDLISFAWQ | |
| | ISQGMQYLAEMKLVHRDLAARNILVAEGRKMKISDEGLSRDVYEEDSYV | |
| | KRSQGRIPVKWMAIESLFDHIYTTQSDVWSFGVLLWEIVTLGGNPYPGI | |
| | PPERLFNLLKTGHRMERPDNCSEEMYRLMLQCWKQEPDKRPVFADISKD | |
| | LEKMMVKRRDYLDLAASTPSDSLIYDDGLSEEETPLVDCNNAPLPRALP | |
| | STWIENKLYGRISHAFTRE | |
| REEP3-RET | MVSWMISRAVVLVFGMLYPAYYSYKAVKTKNVKEYVRWMMYWIVFALYT | 88 |
| | VIETVADQTVAWFPLYYELKIAFVIWLLSPYTKGASLIYRKELHPLLSS | |
| | KEREIDDYIVQAKERGYETMVNEGRQGLNLAATAAVTAAVKEDPKWEFP | |
| | RKNLVLGKTLGEGEFGKVVKATAFHLKGRAGYTTVAVKMLKENASPSEL | |
| | RDLLSEFNVLKQVNHPHVIKLYGACSQDGPLLLIVEYAKYGSLRGELRE | |
| | SRKVGPGYLGSGGSRNSSSLDHPDERALTMGDLISFAWQISQGMQYLAE | |
| | MKLVHRDLAARNILVAEGRKMKISDEGLSRDVYEEDSYVKRSQGRIPVK | |
| | WMAIESLEDHIYTTQSDVWSFGVLLWEIVTLGGNPYPGIPPERLENLLK | |
| | TGHRMERPDNCSEEMYRLMLQCWKQEPDKRPVFADISKDLEKMMVKRRD | |
| | YLDLAASTPSDSLIYDDGLSEEETPLVDCNNAPLPRALPSTWIENKLYG | |
| | RISHAFTRE | |
| RRBP1-RET | MDIYDTQTLGVVVFGGFMVVSAIGIFLVSTESMKETSYEEALANQRKEM | 89 |
| | AKTHHQKVEKKKKEKTVEKKGKTKKKEEKPNGKIPDHDPAPNVTVLLRE | |
| | PVRAPAVAVAPTPVQPPIIVAPVATVPAMPQEKLASSPKDKKKKEKKVA | |
| | KVEPAVSSVVNSIQVLTSKAAILETAPKEGRNTDVAQSPEAPKQEAPAK | |
| | KKSGSKKKGPPDADGPLYLPYKTLVSTVGSMVENEGEAQRLIEILSEKA | |
| | GIIQDTWHKATQKGDPVAILKRQLEEKEKLLATEQEDAAVAKSKLRELN | |
| | KEMAAEKAKAAAGEAKVKKQLVAREQEITAVQARMQASYREHVKEVQQL | |
| | QGKIRTLQEQLENGPNTQLARLQQENSILRDALNQATSQVESKQNAELA | |
| | KLRQELSKVSKELVEKSEAVRQDEQQRKALEAKAAAFEKQVLQLQASHR | |
| | ESEEALQKRLDEVSRELCHTQSSHASLRADAEKAQEQQQQMAELHSKLQ | |
| | SSEAEVRSKCEELSGLHGQLQEARAENSQLTERIRSIEALLEAGQARDA | |
| | QDVQASQAEADQQQTRLKELESQVSGLEKEAIELREAVEQQKVKNNDLR | |
| | EKNWKAMEALATAEQACKEKLLSLTQAKEESEKQLCLIEAQTMEALLAL | |
| | LPELSVLAQQNYTEWLQDLKEKGPTLLKHPPAPAEPSSDLASKLREAEE | |
| | TQSTLQAECDQYRSILAETEGMLRDLQKSVEEEEQVWRAKVGAAEEELQ | |
| | KSRVTVKHLEEIVEKLKGELESSDQVREHTSHLEAELEKHMAAASAECQ | |
| | NYAKEVAGLRQLLLESQSQLDAAKSEAQKQSDELALVRQQLSEMKSHVE | |
| | DGDIAGAPASSPEAPPAEQDPVQLKTQLEWTEAILEDEQTQRQKLTAEF | |
| | EEEDPKWEFPRKNLVLGKTLGEGEFGKVVKATAFHLKGRAGYTTVAVKM | |
| | LKENASPSELRDLLSEFNVLKQVNHPHVIKLYGACSQDGPLLLIVEYAK | |
| | YGSLRGFLRESRKVGPGYLGSGGSRNSSSLDHPDERALTMGDLISFAWQ | |
| | ISQGMQYLAEMKLVHRDLAARNILVAEGRKMKISDFGLSRDVYEEDSYV | |
| | KRSQGRIPVKWMAIESLFDHIYTTQSDVWSFGVLLWEIVTLGGNPYPGI | |
| | PPERLFNLLKTGHRMERPDNCSEEMYRLMLQCWKQEPDKRPVFADISKD | |
| | LEKMMVKRRDYLDLAASTPSDSLIYDDGLSEEETPLVDCNNAPLPRALP | |
| | STWIENKLYGRISHAFTRE | |
| ETV6-RET | MSETPAQCSIKQERISYTPPESPVPSYASSTPLHVPVPRALRMEEDSIR | 90 |
| | LPAHLRLQPIYWSRDDVAQWLKWAENEFSLRPIDSNTFEMNGKALLLLT | |
| | KEDFRYRSPHSGDVLYELLQHILKQRKPRILFSPFFHPGNSIHTQPEVI | |
| | LHQNHEEDNCVQRTPRPSVDNVHHNPPTIELLHRSRSPITTNHRPSPDP | |
| | EQRPLRSPLDNMIRRLSPAERAQGPRPHQENNHQESYPLSVSPMENNHC | |
| | PASSESHPKPSSPRQESTRVIQLMPSPIMHPLILNPRHSVDFKQSRLSE | |
| | DGLHREGKPINLSHREDLAYMNHIMVSVSPPEEHAMPIGRIADCRLLWD | |
| | YVYQLLSDSRYENFIRWEDKESKIFRIVDPNGLARLWGNHKEDPKWEFP | |
| | RKNLVLGKTLGEGEFGKVVKATAFHLKGRAGYTTVAVKMLKENASPSEL | |
| | RDLLSEFNVLKQVNHPHVIKLYGACSQDGPLLLIVEYAKYGSLRGELRE | |
| | SRKVGPGYLGSGGSRNSSSLDHPDERALTMGDLISFAWQISQGMQYLAE | |
| | MKLVHRDLAARNILVAEGRKMKISDEGLSRDVYEEDSYVKRSQGRIPVK | |
| | WMAIESLFDHIYTTQSDVWSFGVLLWEIVTLGGNPYPGIPPERLENLLK | |
| | TGHRMERPDNCSEEMYRLMLQCWKQEPDKRPVFADISKDLEKMMVKRRD | |
| | YLDLAASTPSDSLIYDDGLSEEETPLVDCNNAPLPRALPSTWIENKLYG | |
| | RISHAFTRF | |
| TAF3-RET | MCESYSRSLLRVSVAQICQALGWDSVQLSACHLLTDVLQRYLQQLGRGC | 91 |
| | HRYSELYGRTDPILDDVGEAFQLMGVSLHELEDYIHNIEPVTFPHQIPS | |
| | FPVSKNNVLQFPQPGSKDAEERKEYIPDYLPPIVSSQEEEEEEQVPTDG | |
| | GTSAEAMQVPLEEDDELEEEEIINDENFLGKRPLDSPEAEELPAMKRPR | |
| | LLSTKGDTLDVVLLEAREPLSSINTQKIPPMLSPVHVQDSTDLAPPSPE | |
| | PPMLAPVAKSQMPTAKPLETKSFTPKTKTKTSSPGQKTKSPKTAQSPAM | |
| | VGSPIRSPKTVSKEKKSPGRSKSPKSPKSPKVTTHIPQTPVRPETPNRT | |
| | PSATLSEKISKETIQVKQIQTPPDAGKLNSENQPKKAVVADKTIEASID | |
| | AVIARACAEREPDPFEFSSGSESEGDIFTSPKRISGPECTTPKASTSAN | |
| | NFTKSGSTPLPLSGGTSSSDNSWTMDASIDEVVRKAKLGTPSNMPPNEP | |
| | YISSPSVSPPTPEPLHKVYEEKTKLPSSVEVKKKLKKELKTKMKKKEKQ | |
| | RDREREKDKNKDKSKEKDKVKEKEKDKETGRETKYPWKEFLKEEEADPY | |
| | KFKIKEFEDVDPKVKLKDGLVRKEKEKHKDKKKDREKGKKDKDKREKEK | |
| | VKDKGREDKMKAPAPPLVLPPKELALPLESPATASRVPAMLPSLLPVLP | |
| | EKLFEEKEKVKEKEKKKDKKEKKKKKEKEKEKKEKEREKEKREREKREK | |
| | EKEKHKHEKEDPKWEFPRKNLVLGKTLGEGEFGKVVKATAFHLKGRAGY | |
| | TTVAVKMLKENASPSELRDLLSEFNVLKQVNHPHVIKLYGACSQDGPLL | |
| | LIVEYAKYGSLRGFLRESRKVGPGYLGSGGSRNSSSLDHPDERALTMGD | |
| | LISFAWQISQGMQYLAEMKLVHRDLAARNILVAEGRKMKISDFGLSRDV | |
| | YEEDSYVKRSQGRIPVKWMAIESLFDHIYTTQSDVWSFGVLLWEIVTLG | |
| | GNPYPGIPPERLENLLKTGHRMERPDNCSEEMYRLMLQCWKQEPDKRPV | |
| | FADISKDLEKMMVKRRDYLDLAASTPSDSLIYDDGLSEEETPLVDCNNA | |
| | PLPRALPSTWIENKLYGRISHAFTRF | |
| PCM1-RET | MATGGGPFEDGMNDQDLPNWSNENVDDRLNNMDWGAQQKKANRSSEKNK | 92 |
| | KKFGVESDKRVTNDISPESSPGVGRRRTKTPHTFPHSRYMSQMSVPEQA | |
| | ELEKLKQRINFSDLDQRSIGSDSQGRATAANNKRQLSENRKPENFLPMQ | |
| | INTNKSKDASTNPPNRETIGSAQCKELFASALSNDLLQNCQVSEEDGRG | |
| | EPAMESSQIVSRLVQIRDYITKASSMREDLVEKNERSANVERLTHLIDH | |
| | LKEQEKSYMKELKKILARDPQQEPMEEIENLKKQHDLLKRMLQQQEQLR | |
| | ALQGRQAALLALQHKAEQAIAVMDDSVVAETAGSLSGVSITSELNEELN | |
| | DLIQRFHNQLRDSQPPAVPDNRRQAESLSLTREVSQSRKPSASERLPDE | |
| | KVELFSKMRVLQEKKQKMDKLLGELHTLRDQHLNNSSSSPQRSVDQRST | |
| | SAPSASVGLAPVVNGESNSLTSSVPYPTASLVSQNESENEGHLNPSEKL | |
| | QKLNEVRKRLNELRELVHYYEQTSDMMTDAVNENRKDEETEESEYDSEH | |
| | ENSEPVTNIRNPQVASTWNEVNSHSNAQCVSNNRDGRTVNSNCEINNRS | |
| | AANIRALNMPPSLDCRYNREGEQEIHVAQGEDDEEEEEEEEEGVSGAS | |
| | LSSHRSSLVDEHPEDAEFEQKINRLMAAKQKLRQLQDLVAMVQDDDAAQ | |
| | GVISASASNLDDFYPAEEDTKQNSNNTRGNANKTQKDTGVNEKAREKFY | |
| | EAKLQQQQRELKQLQEERKKLIDIQEKIQALQTACPDLQLSAASVGNCP | |
| | TKKYMPAVTSTPTVNQHETSTSKSVFEPEDSSIVDNELWSEMRRHEMLR | |
| | EELRQRRKQLEALMAEHQRRQGLAETASPVAVSLRSDGSENLCTPQQSR | |
| | TEKTMATWGGSTQCALDEEGDEDGYLSEGIVRTDEEEEEEQDASSNDNE | |
| | SVCPSNSVNHNSYNGKETKNRWKNNCPFSADENYRPLAKTRQQNISMQR | |
| | QENLRWVSELSYVEEKEQWQEQINQLKKQLDFSVSICQTLMQDQQTLSC | |
| | LLQTLLTGPYSVMPSNVASPQVHFIMHQLNQCYTQLTWQQNNVQRLKQM | |
| | LNELMRQQNQHPEKPGGKERGSSASHPPSPSLFCPESEPTQPVNLENIP | |
| | GFTNFSSFAPGMNESPLFPSNFGDFSQNISTPSEQQQPLAQNSSGKTEY | |
| | MAFPKPFESSSSIGAEKPRNKKLPEEEVESSRTPWLYEQEGEVEKPFIK | |
| | TGFSVSVEKSTSSNRKNQLDTNGRRRQFDEESLESESSMPDPVDPTTVT | |
| | KTFKTRKASAQASLASKDKTPKSKSKKRNSTQLKSRVKNIRYESASMSS | |
| | TCEPCKSRNRHSAQTEEPVQAKVFSRKNHEQLEKIIKCNRSTEISSETG | |
| | SDFSMFEALRDTIYSEVATLISQNESRPHELIELFHELQLLNTDYLRQR | |
| | ALYALQDIVSRHISESHEKGENVKSVNSGTWIASNSELTPSESLATTDD | |
| | ETFEKNFERETHKISEQNDADNASVLSVSSNFEPFATDDLGNTVIHLDQ | |
| | ALARMREYERMKTEAESNSNMRCTCRIIEDGDGAGAGTTVNNLEETPVI | |
| | ENRSSQQPVSEVSTIPCPRIDTQQLDRQIKAIMKEVIPELKEDPKWEFP | |
| | RKNLVLGKTLGEGEFGKVVKATAFHLKGRAGYTTVAVKMLKENASPSEL | |
| | RDLLSEFNVLKQVNHPHVIKLYGACSQDGPLLLIVEYAKYGSLRGELRE | |
| | SRKVGPGYLGSGGSRNSSSLDHPDERALTMGDLISFAWQISQGMQYLAE | |
| | MKLVHRDLAARNILVAEGRKMKISDFGLSRDVYEEDSYVKRSQGRIPVK | |
| | WMAIESLEDHIYTTQSDVWSFGVLLWEIVTLGGNPYPGIPPERLENLLK | |
| | TGHRMERPDNCSEEMYRLMLQCWKQEPDKRPVFADISKDLEKMMVKRRD | |
| | YLDLAASTPSDSLIYDDGLSEEETPLVDCNNAPLPRALPSTWIENKLYG | |
| | RISHAFTRF | |
| PCM1-RET | MATGGGPFEDGMNDQDLPNWSNENVDDRLNNMDWGAQQKKANRSSEKNK | 93 |
| | KKFGVESDKRVTNDISPESSPGVGRRRTKTPHTFPHSRYMSQMSVPEQA | |
| | ELEKLKQRINFSDLDQRSIGSDSQGRATAANNKRQLSENRKPENFLPMQ | |
| | INTNKSKDASTNPPNRETIGSAQCKELFASALSNDLLQNCQVSEEDGRG | |
| | EPAMESSQIVSRLVQIRDYITKASSMREDLVEKNERSANVERLTHLIDH | |
| | LKEQEKSYMKFLKKILARDPQQEPMEEIENLKKQHDLLKRMLQQQEQLR | |
| | ALQGRQAALLALQHKAEQAIAVMDDSVVAETAGSLSGVSITSELNEELN | |
| | DLIQRFHNQLRDSQPPAVPDNRRQAESLSLTREVSQSRKPSASERLPDE | |
| | KVELFSKMRVLQEKKQKMDKLLGELHTLRDQHLNNSSSSPQRSVDQRST | |
| | SAPSASVGLAPVVNGESNSLTSSVPYPTASLVSQNESENEGHLNPSEKL | |
| | QKLNEVRKRLNELRELVHYYEQTSDMMTDAVNENRKDEETEESEYDSEH | |
| | ENSEPVTNIRNPQVASTWNEVNSHSNAQCVSNNRDGRTVNSNCEINNRS | |
| | AANIRALNMPPSLDCRYNREGEQEIHVAQGEDDEEEEEEAEEEGVSGAS | |
| | LSSHRSSLVDEHPEDAEFEQKINRLMAAKQKLRQLQDLVAMVQDDDAAQ | |
| | GVISASASNLDDFYPAEEDTKQNSNNTRGNANKTQKDTGVNEKAREKFY | |
| | EAKLQQQQRELKQLQEERKKLIDIQEKIQALQTACPDLQLSAASVGNCP | |
| | TKKYMPAVTSTPTVNQHETSTSKSVFEPEDSSIVDNELWSEMRRHEMLR | |
| | EELRQRRKQLEALMAEHQRRQGLAETASPVAVSLRSDGSENLCTPQQSR | |
| | TEKTMATWGGSTQCALDEEGDEDGYLSEGIVRTDEEEEEEQDASSNDNE | |
| | SVCPSNSVNHNSYNGKETKNRWKNNCPFSADENYRPLAKTRQQNISMQR | |
| | QENLRWVSELSYVEEKEQWQEQINQLKKQLDFSVSICQTLMQDQQTLSC | |
| | LLQTLLTGPYSVMPSNVASPQVHFIMHQLNQCYTQLTWQQNNVQRLKQM | |
| | LNELMRQQNQHPEKPGGKERGSSASHPPSPSLFCPFSFPTQPVNLENIP | |
| | GFTNESSFAPGMNESPLFPSNEGDESQNISTPSEQQQPLAQNSSGKTEY | |
| | MAFPKPFESSSSIGAEKPRNKKLPEEEVESSRTPWLYEQEGEVEKPFIK | |
| | TGFSVSVEKSTSSNRKNQLDTNGRRRQFDEESLESFSSMPDPVDPTTVT | |
| | KTFKTRKASAQASLASKDKTPKSKSKKRNSTQLKSRVKNIRYESASMSS | |
| | TCEPCKSRNRHSAQTEEPVQAKVESRKNHEQLEKIIKCNRSTEISSETG | |
| | SDESMFEALRDTIYSEVATLISQNESRPHELIELFHELQLLNTDYLRQR | |
| | ALYALQDIVSRHISESHEKGENVKSVNSGTWIASNSELTPSESLATTDD | |
| | ETFEKNFERETHKISEQNDADNASVLSVSSNFEPFATDDLGNTVIHLDQ | |
| | ALARMREYERMKTEAESNSNMRCTCRIIEDGDGAGAGTTVNNLEETPVI | |
| | ENRSSQQPVSEVSTIPCPRIDTQQLDRQIKAIMKEVIPELKEDPKWEFP | |
| | RKNLVLGKTLGEGEFGKVVKATAFHLKGRAGYTTVAVKMLKENASPSEL | |
| | RDLLSEFNVLKQVNHPHVIKLYGACSQDGPLLLIVEYAKYGSLRGELRE | |
| | SRKVGPGYLGSGGSRNSSSLDHPDERALTMGDLISFAWQISQGMQYLAE | |
| | MKLVHRDLAARNILVAEGRKMKISDFGLSRDVYEEDSYVKRSQGRIPVK | |
| | WMAIESLFDHIYTTQSDVWSFGVLLWEIVTLGGNPYPGIPPERLENLLK | |
| | TGHRMERPDNCSEEMYRLMLQCWKQEPDKRPVFADISKDLEKMMVKRRD | |
| | YLDLAASTPSDSLIYDDGLSEEETPLVDCNNAPLPRALPSTWIENKLYG | |
| | RISHAFTRE | |
| TNIP2-RET | MSRDPGSGGWEEAPRAAAALCTLYHEAGQRLRRLQDQLAARDALIARLR | 94 |
| | ARLAALEGDAAPSLVDALLEQVARFREQLRRQEGGAAEAQMRQEIERLT | |
| | ERLEEKEREMQQLLSQPQHEREKEVVLLRRSMAEGERARAASDVLCRSL | |
| | ANETHQLRRTLTATAHMCQHLAKCLDERQHAQRNVGERSPDQSEHTDGH | |
| | TSVQSVIEKLQEENRLLKQKVTHVEDLNAKWQRYNASRDEYVRGLHAQL | |
| | RGLQIPHEPELMRKEISRLNRQLEEKINDCAEVKQELAASRTARDAALE | |
| | RVQMLEQQILAYKDDEMSERADRERAQSRIQELEEKVASLLHQVSWRQE | |
| | DPKWEFPRKNLVLGKTLGEGEFGKVVKATAFHLKGRAGYTTVAVKMLKE | |
| | NASPSELRDLLSEENVLKQVNHPHVIKLYGACSQDGPLLLIVEYAKYGS | |
| | LRGFLRESRKVGPGYLGSGGSRNSSSLDHPDERALTMGDLISFAWQISQ | |
| | GMQYLAEMKLVHRDLAARNILVAEGRKMKISDEGLSRDVYEEDSYVKRS | |
| | QGRIPVKWMAIESLFDHIYTTQSDVWSFGVLLWEIVTLGGNPYPGIPPE | |
| | RLFNLLKTGHRMERPDNCSEEMYRLMLQCWKQEPDKRPVFADISKDLEK | |
| | MMVKRRDYLDLAASTPSDSLIYDDGLSEEETPLVDCNNAPLPRALPSTW | |
| | IENKLYGRISHAFTRF | |
| SATB1-RET | MDHLNEATQGKEHSEMSNNVSDPKGPPAKIARLEQNGSPLGRGRLGSTG | 95 |
| | AKMQGVPLKHSGHLMKTNLRKGTMLPVFCVVEHYENAIEYDCKEEHAEF | |
| | VLVRKDMLFNQLIEMALLSLGYSHSSAAQAKGLIQVGKWNPVPLSYVTD | |
| | APDATVADMLQDVYHVVTLKIQLHSCPKLEDLPPEQWSHTTVRNALKDL | |
| | LKDMNQSSLAKECPLSQSMISSIVNSTYYANVSAAKCQEFGRWYKHEKK | |
| | TKDMMVEMDSLSELSQQGANHVNFGQQPVPGNTAEQPPSPAQLSHGSQP | |
| | SVRTPLPNLHPGLVSTPISPQLVNQQLVMAQLLNQQYAVNRLLAQQSLN | |
| | QQYLNHPPPVSRSMNKPLEQQVSTNTEVSSEIYQWVRDELKRAGISQAV | |
| | FARVAFNRTQEDPKWEFPRKNLVLGKTLGEGEFGKVVKATAFHLKGRAG | |
| | YTTVAVKMLKENASPSELRDLLSEFNVLKQVNHPHVIKLYGACSQDGPL | |
| | LLIVEYAKYGSLRGFLRESRKVGPGYLGSGGSRNSSSLDHPDERALTMG | |
| | DLISFAWQISQGMQYLAEMKLVHRDLAARNILVAEGRKMKISDFGLSRD | |
| | VYEEDSYVKRSQGRIPVKWMAIESLFDHIYTTQSDVWSFGVLLWEIVTL | |
| | GGNPYPGIPPERLFNLLKTGHRMERPDNCSEEMYRLMLQCWKQEPDKRP | |
| | VFADISKDLEKMMVKRRDYLDLAASTPSDSLIYDDGLSEEETPLVDCNN | |
| | APLPRALPSTWIENKLYGRISHAFTRE | |
| RET-ADCY1 | MAKATSGAAGLRLLLLLLLPLLGKVALGLYFSRDAYWEKLYVDQAAGTP | 96 |
| | LLYVHALRDAPEEVPSFRLGQHLYGTYRTRLHENNWICIQEDTGLLYLN | |
| | RSLDHSSWEKLSVRNRGFPLLTVYLKVFLSPTSLREGECQWPGCARVYF | |
| | SFFNTSFPACSSLKPRELCFPETRPSFRIRENRPPGTFHQFRLLPVQFL | |
| | CPNISVAYRLLEGEGLPFRCAPDSLEVSTRWALDREQREKYELVAVCTV | |
| | HAGAREEVVMVPFPVTVYDEDDSAPTFPAGVDTASAVVEFKRKEDTVVA | |
| | TLRVFDADVVPASGELVRRYTSTLLPGDTWAQQTERVEHWPNETSVQAN | |
| | GSFVRATVHDYRLVLNRNLSISENRTMQLAVLVNDSDFQGPGAGVLLLH | |
| | FNVSVLPVSLHLPSTYSLSVSRRARRFAQIGKVCVENCQAFSGINVQYK | |
| | LHSSGANCSTLGVVTSAEDTSGILFVNDTKALRRPKCAELHYMVVATDQ | |
| | QTSRQAQAQLLVTVEGSYVAEEAGCPLSCAVSKRRLECEECGGLGSPTG | |
| | RCEWRQGDGKGITRNFSTCSPSTKTCPDGHCDVVETQDINICPQDCLRG | |
| | SIVGGHEPGEPRGIKAGYGTCNCFPEEEKCFCEPEDIQDPLCDELCRTV | |
| | IAAAVLESFIVSVLLSAFCIHCYHKFAHKPPISSAEMTERRPAQAFPVS | |
| | YSSSGARRPSLDSMENQVSVDAFKILCFPGCLTIQIRTVLCIFIVVLIY | |
| | SVAQGCVVGCLPWAWSSKPNSSLVVLSSGGQRTALPTLPCESTHHALLC | |
| | CLVGTLPLAIFFRVSSLPKMILLSGLTTSYILVLELSGYTRTGGGAVSG | |
| | RSYEPIVAILLESCALALHARQVDIRLRLDYLWAAQAEEEREDMEKVKL | |
| | DNRRILENLLPAHVAQHELMSNPRNMDLYYQSYSQVGVMFASIPNENDE | |
| | YIELDGNNMGVECLRLLNEIIADEDELMEKDFYKDIEKIKTIGSTYMAA | |
| | VGLAPTSGTKAKKSISSHLSTLADFAIEMEDVLDEINYQSYNDFVLRVG | |
| | INVGPVVAGVIGARRPQYDIWGNTVNVASRMDSTGVQGRIQVTEEVHRL | |
| | LRRCPYHFVCRGKVSVKGKGEMLTYFLEGRTDGNGSQIRSLGLDRKMCP | |
| | FGRAGLQGRRPPVCPMPGVSVRAGLPPHSPGQYLPSAAAGKEA | |
| RET-ZNF248 | MAKATSGAAGLRLLLLLLLPLLGKVALGLYFSRDAYWEKLYVDQAAGTP | 97 |
| | LLYVHALRDAPEEVPSFRLGQHLYGTYRTRLHENNWICIQEDTGLLYLN | |
| | RSLDHSSWEKLSVRNRGFPLLTVYLKVELSPTSLREGECQWPGCARVYF | |
| | SFFNTSFPACSSLKPRELCFPETRPSFRIRENRPPGTFHQFRLLPVQFL | |
| | CPNISVAYRLLEGEGLPERCAPDSLEVSTRWALDREQREKYELVAVCTV | |
| | HAGAREEVVMVPFPVTVYDEDDSAPTFPAGVDTASAVVEFKRKEDTVVA | |
| | TLRVFDADVVPASGELVRRYTSTLLPGDTWAQQTERVEHWPNETSVQAN | |
| | GSFVRATVHDYRLVLNRNLSISENRTMQLAVLVNDSDFQGPGAGVLLLH | |
| | FNVSVLPVSLHLPSTYSLSVSRRARRFAQIGKVCVENCQAFSGINVQYK | |
| | LHSSGANCSTLGVVTSAEDTSGILFVNDTKALRRPKCAELHYMVVATDQ | |
| | QTSRQAQAQLLVTVEGSYVAEEAGCPLSCAVSKRRLECEECGGLGSPTG | |
| | RCEWRQGDGKGITRNFSTCSPSTKTCPDGHCDVVETQDINICPQDCLRG | |
| | SIVGGHEPGEPRGIKAGYGTCNCFPEEEKCFCEPEDIQDPLCDELCRTV | |
| | IAAAVLESFIVSVLLSAFCIHCYHKFAHKPPISSAEMTERRPAQAFPVS | |
| | YSSSGARRPSLDSMENQVSVDAFKILEQVSFKDVCVDFTQEEWYLLDPA | |
| | QKILYRDVILENYSNLVSVGYCITKPEVIFKIEQGEEPWILEKGFPSQC | |
| | HPERKWKVDDVLESSQENEDDHFWELLFHNNKTVSVENGDRGSKTENLG | |
| | TDPVSLRNYPYKICDSCEMNLKNISGLIISKKNCSRKKPDEFNVCEKLL | |
| | LDIRHEKIPIGEKSYKYDQKRNAINYHQDLSQPSFGQSFEYSKNGQGEH | |
| | DEAAFFTNKRSQIGETVCKYNECGRTFIESLKLNISQRPHLEMEPYGCS | |
| | ICGKSFCMNLRFGHQRALTKDNPYEYNEYGEIFCDNSAFIIHQGAYTRK | |
| | ILREYKVSDKTWEKSALLKHQIVHMGGKSYDYNENGSNFSKKSHLTQLR | |
| | RAHTGEKTFECGECGKTFWEKSNLTQHQRTHTGEKPYECTECGKAFCQK | |
| | PHLTNHQRTHTGEKPYECKQCGKTFCVKSNLTEHQRTHTGEKPYECNAC | |
| | GKSFCHRSALTVHQRTHTGEKPFICNECGKSFCVKSNLIVHQRTHTGEK | |
| | PYKCNECGKTFCEKSALTKHQRTHTGEKPYECNACGKTFSQRSVLTKHQ | |
| | RIHTRVKALSTS | |
| RET-AGBL4 | MAKATSGAAGLRLLLLLLLPLLGKVALGLYFSRDAYWEKLYVDQAAGTP | 98 |
| | LLYVHALRDAPEEVPSFRLGQHLYGTYRTRLHENNWICIQEDTGLLYLN | |
| | RSLDHSSWEKLSVRNRGFPLLTVYLKVELSPTSLREGECQWPGCARVYF | |
| | SFFNTSFPACSSLKPRELCFPETRPSFRIRENRPPGTFHQFRLLPVQFL | |
| | CPNISVAYRLLEGEGLPFRCAPDSLEVSTRWALDREQREKYELVAVCTV | |
| | HAGAREEVVMVPFPVTVYDEDDSAPTFPAGVDTASAVVEFKRKEDTVVA | |
| | TLRVFDADVVPASGELVRRYTSTLLPGDTWAQQTERVEHWPNETSVQAN | |
| | GSFVRATVHDYRLVLNRNLSISENRTMQLAVLVNDSDFQGPGAGVLLLH | |
| | FNVSVLPVSLHLPSTYSLSVSRRARRFAQIGKVCVENCQAFSGINVQYK | |
| | LHSSGANCSTLGVVTSAEDTSGILFVNDTKALRRPKCAELHYMVVATDQ | |
| | QTSRQAQAQLLVTVEGSYVAEEAGCPLSCAVSKRRLECEECGGLGSPTG | |
| | RCEWRQGDGKGITRNESTCSPSTKTCPDGHCDVVETQDINICPQDCLRG | |
| | SIVGGHEPGEPRGIKAGYGTCNCFPEEEKCFCEPEDIQDPLCDELCRTV | |
| | IAAAVLFSFIVSVLLSAFCIHCYHKFAHKPPISSAEMTERRPAQAFPVS | |
| | YSSSGARRPSLDSMENQVSVDAFKILEDPKWEFPRKNLVLGKTLGEGEF | |
| | GKVVKATAFHLKGRAGYTTVAVKMLKGNLGRVDQVSEFEYDLFIRPDTC | |
| | NPRFRVWFNFTVENVKESQRVIFNIVNFSKTKSLYRDGMAPMVKSTSRP | |
| | KWQRLPPKNVYYYRCPDHRKNYVMSFAFCFDREEDIYQFAYCYPYTYTR | |
| | FQHYLDSLQKRNMDYFFREQLGQSVQQRKLDLLTITSPDNLREGAEQKV | |
| | VFITGRVHPGETPSSFVCQGIIDFLVSQHPIACVLREYLVFKIAPMLNP | |
| | DGVYLGNYRCSLMGFDLNRHWLDPSPWVHPTLHGVKQLIVQMYNDPKTS | |
| | LEFYIDIHAHSTMMNGFMYGNIFEDEERFQRQAIFPKLLCQNAEDESYS | |
| | STSFNRDAVKAGTGRRFLGGLLDHTSYCYTLEVSFYSYIISGTTAAVPY | |
| | TEEAYMKLGRNVARTFLDYYRLNPVVEKVAIPMPRLRNKEIEVQRRKEK | |
| | SPPYKHPLLRGPASNYPNSKGDKKSSVNHKDPSTPF | |
| RET-LRMDA | MAKATSGAAGLRLLLLLLLPLLGKVALGLYFSRDAYWEKLYVDQAAGTP | 99 |
| | LLYVHALRDAPEEVPSFRLGQHLYGTYRTRLHENNWICIQEDTGLLYLN | |
| | RSLDHSSWEKLSVRNRGFPLLTVYLKVELSPTSLREGECQWPGCARVYF | |
| | SFFNTSFPACSSLKPRELCFPETRPSFRIRENRPPGTFHQFRLLPVQFL | |
| | CPNISVAYRLLEGEGLPFRCAPDSLEVSTRWALDREQREKYELVAVCTV | |
| | HAGAREEVVMVPFPVTVYDEDDSAPTFPAGVDTASAVVEFKRKEDTVVA | |
| | TLRVFDADVVPASGELVRRYTSTLLPGDTWAQQTERVEHWPNETSVQAN | |
| | GSFVRATVHDYRLVLNRNLSISENRTMQLAVLVNDSDFQGPGAGVLLLH | |
| | FNVSVLPVSLHLPSTYSLSVSRRARRFAQIGKVCVENCQAFSGINVQYK | |
| | LHSSGANCSTLGVVTSAEDTSGILFVNDTKALRRPKCAELHYMVVATDQ | |
| | QTSRQAQAQLLVTVEGSYVAEEAGCPLSCAVSKRRLECEECGGLGSPTG | |
| | RCEWRQGDGKGITRNFSTCSPSTKTCPDGHCDVVETQDINICPQDCLRG | |
| | SIVGGHEPGEPRGIKAGYGTCNCFPEEEKCFCEPEDIQGVLGKCRYVYY | |
| | GKNSEGNRFIRDDQL | |
| RET- | MAKATSGAAGLRLLLLLLLPLLGKVALGLYFSRDAYWEKLYVDQAAGTP | 100 |
| ARHGAP19 | LLYVHALRDAPEEVPSFRLGQHLYGTYRTRLHENNWICIQEDTGLLYLN | |
| | RSLDHSSWEKLSVRNRGFPLLTVYLKVELSPTSLREGECQWPGCARVYF | |
| | SFFNTSFPACSSLKPRELCFPETRPSFRIRENRPPGTFHQFRLLPVQFL | |
| | CPNISVAYRLLEGEGLPFRCAPDSLEVSTRWALDREQREKYELVAVCTV | |
| | HAGAREEVVMVPFPVTVYDEDDSAPTEPAGVDTASAVVEFKRKEDTVVA | |
| | TLRVFDADVVPASGELVRRYTSTLLPGDTWAQQTERVEHWPNETSVQAN | |
| | GSFVRATVHDYRLVLNRNLSISENRTMQLAVLVNDSDFQGPGAGVLLLH | |
| | FNVSVLPVSLHLPSTYSLSVSRRARRFAQIGKVCVENCQAFSGINVQYK | |
| | LHSSGANCSTLGVVTSAEDTSGILFVNDTKALRRPKCAELHYMVVATDQ | |
| | QTSRQAQAQLLVTVEGSYVAEEAGCPLSCAVSKRRLECEECGGLGSPTG | |
| | RCEWRQGDGKGITRNFSTCSPSTKTCPDGHCDVVETQDINICPQDCLRG | |
| | SIVGGHEPGEPRGIKAGYGTCNCFPEEEKCFCEPEDIQDPLCDELCRTV | |
| | IAAAVLESFIVSVLLSAFCIHCYHKFAHKPPISSAEMTERRPAQAFPVS | |
| | YSSSGARRPSLDSMENQVSVDAFKILAPAYIRECARLHYLGSRTQASKD | |
| | DLDLIASCHTKSFQLAKSQKRNRVDSCPHQEETQHHTEEALRELFQHVH | |
| | DMPESAKKKQLIRQFNKQSLTQTPGREPSTSQVQKRARSRSFSGLIKRK | |
| | VLGNQMMSEKKKKNPTPESVAIGELKGTSKENRNLLESGSPAVTMTPTR | |
| | LKWSEGKKEGKKGEL | |
| RET-CPEB3 | MAKATSGAAGLRLLLLLLLPLLGKVALGLYFSRDAYWEKLYVDQAAGTP | 101 |
| | LLYVHALRDAPEEVPSFRLGQHLYGTYRTRLHENNWICIQEDTGLLYLN | |
| | RSLDHSSWEKLSVRNRGFPLLTVYLKVFLSPTSLREGECQWPGCARVYF | |
| | SFFNTSFPACSSLKPRELCFPETRPSFRIRENRPPGTFHQFRLLPVQFL | |
| | CPNISVAYRLLEGEGLPERCAPDSLEVSTRWALDREQREKYELVAVCTV | |
| | HAGAREEVVMVPFPVTVYDEDDSAPTFPAGVDTASAVVEFKRKEDTVVA | |
| | TLRVEDADVVPASGELVRRYTSTLLPGDTWAQQTERVEHWPNETSVQAN | |
| | GSFVRATVHDYRLVLNRNLSISENRTMQLAVLVNDSDFQGPGAGVLLLH | |
| | FNVSVLPVSLHLPSTYSLSVSRRARRFAQIGKVCVENCQAFSGINVQYK | |
| | LHSSGANCSTLGVVTSAEDTSGILFVNDTKALRRPKCAELHYMVVATDQ | |
| | QTSRQAQAQLLVTVEGSYVAEEAGCPLSCAVSKRRLECEECGGLGSPTG | |
| | RCEWRQGDGKGITRNFSTCSPSTKTCPDGHCDVVETQDINICPQDCLRG | |
| | SIVGGHEPGEPRGIKAGYGTCNCFPEEEKCFCEPEDIQDPLCDELCRTV | |
| | IAAAVLESFIVSVLLSAFCIHCYHKFAHKPPISSAEMTERRPAQAFPVS | |
| | YSSSGARRPSLDSMENQVSVDAFKILVQIRPWNLSDSDFVMDGSQPLDP | |
| | RKTIFVGGVPRPLRAVELAMIMDRLYGGVCYAGIDTDPELKYPKGAGRV | |
| | AFSNQQSYIAAISARFVQLQHNDIDKRVEVKPYVLDDQMCDECQGTRCG | |
| | GKFAPFFCANVTCLQYYCEYCWASIHSRAGREFHKPLVKEGGDRPRHVP | |
| | FRWS | |
| RET-DCC | MAKATSGAAGLRLLLLLLLPLLGKVALGLYFSRDAYWEKLYVDQAAGTP | 102 |
| | LLYVHALRDAPEEVPSFRLGQHLYGTYRTRLHENNWICIQEDTGLLYLN | |
| | RSLDHSSWEKLSVRNRGFPLLTVYLKVELSPTSLREGECQWPGCARVYF | |
| | SFFNTSFPACSSLKPRELCFPETRPSFRIRENRPPGTFHQFRLLPVQFL | |
| | CPNISVAYRLLEGEGLPFRCAPDSLEVSTRWALDREQREKYELVAVCTV | |
| | HAGAREEVVMVPFPVTVYDEDDSAPTFPAGVDTASAVVEFKRKEDTVVA | |
| | TLRVFDADVVPASGELVRRYTSTLLPGDTWAQQTERVEHWPNETSVQAN | |
| | GSFVRATVHDYRLVLNRNLSISENRTMQLAVLVNDSDFQGPGAGVLLLH | |
| | FNVSVLPVSLHLPSTYSLSVSRRARRFAQIGKVCVENCQAFSGINVQYK | |
| | LHSSGANCSTLGVVTSAEDTSGILFVNDTKALRRPKCAELHYMVVATDQ | |
| | QTSRQAQAQLLVTVEGSYVAEEAGCPLSCAVSKRRLECEECGGLGSPTG | |
| | RCEWRQGDGKGITRNFSTCSPSTKTCPDGHCDVVETQDINICPQDCLPI | |
| | PSSSVLPSAPRDVVPVLVSSRFVRLSWRPPAEAKGNIQTFTVFFSREGD | |
| | NRERALNTTQPGSLQLTVGNLKPEAMYTERVVAYNEWGPGESSQPIKVA | |
| | TQPELQVPGPVENLQAVSTSPTSILITWEPPAYANGPVQGYRLFCTEVS | |
| | TGKEQNIEVDGLSYKLEGLKKFTEYSLRFLAYNRYGPGVSTDDITVVTL | |
| | SDVPSAPPQNVSLEVVNSRSIKVSWLPPPSGTQNGFITGYKIRHRKTTR | |
| | RGEMETLEPNNLWYLFTGLEKGSQYSFQVSAMTVNGTGPPSNWYTAETP | |
| | ENDLDESQVPDQPSSLHVRPQTNCIIMSWTPPLNPNIVVRGYIIGYGVG | |
| | SPYAETVRVDSKQRYYSIERLESSSHYVISLKAFNNAGEGVPLYESATT | |
| | RSITDPTDPVDYYPLLDDFPTSVPDLSTPMLPPVGVQAVALTHDAVRVS | |
| | WADNSVPKNQKTSEVRLYTVRWRTSFSASAKYKSEDTTSLSYTATGLKP | |
| | NTMYEFSVMVTKNRRSSTWSMTAHATTYEAAPTSAPKDLTVITREGKPR | |
| | AVIVSWQPPLEANGKITAYILFYTLDKNIPIDDWIMETISGDRLTHQIM | |
| | DLNLDTMYYFRIQARNSKGVGPLSDPILFRTLKVEHPDKMANDQGRHGD | |
| | GGYWPVDTNLIDRSTLNEPPIGQMHPPHGSVTPQKNSNLLVIIVVTVGV | |
| | ITVLVVVIVAVICTRRSSAQQRKKRATHSAGKRKGSQKDLRPPDLWIHH | |
| | EEMEMKNIEKPSGTDPAGRDSPIQSCQDLTPVSHSQSETQLGSKSTSHS | |
| | GQDTEEAGSSMSTLERSLAARRAPRAKLMIPMDAQSNNPAVVSAIPVPT | |
| | LESAQYPGILPSPTCGYPHPQFTLRPVPFPTLSVDRGFGAGRSQSVSEG | |
| | PTTQQPPMLPPSQPEHSSSEEAPSRTIPTACVRPTHPLRSFANPLLPPP | |
| | MSAIEPKVPYTPLLSQPGPTLPKTHVKTASLGLAGKARSPLLPVSVPTA | |
| | PEVSEESHKPTEDSANVYEQDDLSEQMASLEGLMKQLNAITGSAF | |
| RET-ELMO1 | MAKATSGAAGLRLLLLLLLPLLGKVALGLYFSRDAYWEKLYVDQAAGTP | 103 |
| | LLYVHALRDAPEEVPSFRLGQHLYGTYRTRLHENNWICIQEDTGLLYLN | |
| | RSLDHSSWEKLSVRNRGFPLLTVYLKVELSPTSLREGECQWPGCARVYF | |
| | SFFNTSFPACSSLKPRELCFPETRPSFRIRENRPPGTFHQFRLLPVQFL | |
| | CPNISVAYRLLEGEGLPFRCAPDSLEVSTRWALDREQREKYELVAVCTV | |
| | HAGAREEVVMVPFPVTVYDEDDSAPTFPAGVDTASAVVEFKRKEDTVVA | |
| | TLRVFDADVVPASGELVRRYTSTLLPGDTWAQQTERVEHWPNETSVQAN | |
| | GSFVRATVHDYRLVLNRNLSISENRTMQLAVLVNDSDFQGPGAGVLLLH | |
| | FNVSVLPVSLHLPSTYSLSVSRRARRFAQIGKVCVENCQAFSGINVQYK | |
| | LHSSGANCSTLGVVTSAEDTSGILFVNDTKALRRPKCAELHYMVVATDQ | |
| | QTSRQAQAQLLVTVEGSYVAEEAGCPLSCAVSKRRLECEECGGLGSPTG | |
| | RCEWRQGDGKGITRNFSTCSPSTKTCPDGHCDVVETQDINICPQDCLRG | |
| | SIVGGHEPGEPRGIKAGYGTCNCFPEEEKCFCEPEDIQDPLCDELCRTV | |
| | IAAAVLESFIVSVLLSAFCIHCYHKFAHKPPISSAEMTERRPAQAFPVS | |
| | YSSSGARRPSLDSMENQVSVDAFKILNHVNPAMDFTQTPPGMLALDNML | |
| | YFAKHHQDAYIRIVLENSSREDKHECPFGRSSIELTKMLCEILKVGELP | |
| | SETCNDFHPMFFTHDRSFEEFFCICIQLLNKTWKEMRATSEDENKVMQV | |
| | VKEQVMRALTTKPSSLDQFKSKLQNLSYTEILKIRQSERMNQEDFQSRP | |
| | ILELKEKIQPEILELIKQQRLNRLVEGTCFRKLNARRRQDKFWYCRLSP | |
| | NHKVLHYGDLEESPQGEVPHDSLQDKLPVADIKAVVTGKDCPHMKEKGA | |
| | LKQNKEVLELAFSILYDSNCQLNFIAPDKHEYCIWTDGLNALLGKDMMS | |
| | DLTRNDLDTLLSMEIKLRLLDLENIQIPDAPPPIPKEPSNYDEVYDCN | |
| RET-WDFY4 | MAKATSGAAGLRLLLLLLLPLLGKVALGLYFSRDAYWEKLYVDQAAGTP | 104 |
| | LLYVHALRDAPEEVPSFRLGQHLYGTYRTRLHENNWICIQEDTGLLYLN | |
| | RSLDHSSWEKLSVRNRGFPLLTVYLKVELSPTSLREGECQWPGCARVYF | |
| | SFFNTSFPACSSLKPRELCFPETRPSFRIRENRPPGTFHQFRLLPVQFL | |
| | CPNISVAYRLLEGEGLPFRCAPDSLEVSTRWALDREQREKYELVAVCTV | |
| | HAGAREEVVMVPFPVTVYDEDDSAPTFPAGVDTASAVVEFKRKEDTVVA | |
| | TLRVEDADVVPASGELVRRYTSTLLPGDTWAQQTERVEHWPNETSVQAN | |
| | GSFVRATVHDYRLVLNRNLSISENRTMQLAVLVNDSDFQGPGAGVLLLH | |
| | FNVSVLPVSLHLPSTYSLSVSRRARRFAQIGKVCVENCQAFSGINVQYK | |
| | LHSSGANCSTLGVVTSAEDTSGILFVNDTKALRRPKCAELHYMVVATDQ | |
| | QTSRQAQAQLLVTVEGSYVAEEAGCPLSCAVSKRRLECEECGGLGSPTG | |
| | RCEWRQGDGKGITRNFSTCSPSTKTCPDGHCDVVETQDINICPQDCLRG | |
| | SIVGGHEPGEPRGIKAGYGTCNCFPEEEKCFCEPEDIQDPLCDELCRTV | |
| | IAAAVLESFIVSVLLSAFCIHCYHKFAHKPPISSAEMTERRPAQAFPVS | |
| | YSSSGARRPSLDSMENQVSVDAFKILEDPKWEFPRKNLVLGKTLGEGEF | |
| | GKVVKATAFHLKGRAGYTTVAVKMLKENASPSELRDLLSEFNVLKQVNH | |
| | PHVIKLYGACSQDGPLLLIVEYAKYGSLRGFLRESRKVGPGYLGSGGSR | |
| | NSSSLDHPDERALTMGDLISFAWQISQGMQYLAEMKLVHRDLAARNILV | |
| | AEGRKMKISDFGLSRDVYEEDSYVKRSQDFVSCIENYRRRGQELYASLY | |
| | KDHVQRRKCGNIKAANAWARIQEQLFGELGLWSQGEETKPCSPWELDWR | |
| | EGPARMRKRIKRLSPLEALSSGRHKESQDKNDHISQTNAENQDELTLRE | |
| | AEGEPDEVGVDCTQLTFFPALHESLHSEDFLELCRERQVILQELLDKEK | |
| | VTQKFSLVIVQGHLVSEGVLLFGHQHFYICENFTLSPTGDVYCTRHCLS | |
| | NISDPFIFNLCSKDRSTDHYSCQCHSYADMRELRQARELLQDIALEIFF | |
| | HNGYSKFLVFYNNDRSKAFKSFCSFQPSLKGKATSEDTLSLRRYPGSDR | |
| | IMLQKWQKRDISNFEYLMYLNTAAGRTCNDYMQYPVFPWVLADYTSETL | |
| | NLANPKIFRDLSKPMGAQTKERKLKFIQRFKEVEKTEGDMTVQCHYYTH | |
| | YSSAIIVASYLVRMPPFTQAFCALQGGSFDVADRMFHSVKSTWESASRE | |
| | NMSDVRELTPEFFYLPEFLTNCNGVEFGCMQDGTVLGDVQLPPWADGDP | |
| | RKFISLHRKALESDFVSANLHHWIDLIFGYKQQGPAAVDAVNIFHPYFY | |
| | GDRMDLSSITDPLIKSTILGFVSNFGQVPKQLFTKPHPARTAAGKPLPG | |
| | KDVSTPVSLPGHPQPFFYSLQSLRPSQVTVKDMYLESLGSESPKGAIGH | |
| | IVSTEKTILAVERNKVLLPPLWNRTFSWGEDDESCCLGSYGSDKVLMTF | |
| | ENLAAWGRCLCAVCPSPTTIVTSGTSTVVCVWELSMTKGRPRGLRLRQA | |
| | LYGHTQAVTCLAASVTFSLLVSGSQDCTCILWDLDHLTHVTRLPAHREG | |
| | ISAITISDVSGTIVSCAGAHLSLWNVNGQPLASITTAWGPEGAITCCCL | |
| | MEGPAWDTSQIIITGSQDGMVRVWKTEDVKMSVPGRPAGEEPPAQPPSP | |
| | RGHKWEKNLALSRELDVSIALTGKPSKTSPAVTALAVSRNHTKLLVGDE | |
| | RGRIFCWSADG | |
| ANKRD26- | MKKIFSKKGESPLGSFARRQRSSAGGGGEPGEGAYSQPGYHVRDRDLGK | 105 |
| RET | IHKAASAGNVAKVQQILLLRKNGLNDRDKMNRTALHLACANGHPEVVTL | |
| | LVDRKCQLNVCDNENRTALMKAVQCQEEKCATILLEHGADPNLADVHGN | |
| | TALHYAVYNEDISVATKLLLYDANIEAKNKDDLTPLLLAVSGKKQQMVE | |
| | FLIKKKANVNAVDKLESSHQLISEYKEERIPKHSSQNSNSVDESSEDSL | |
| | SRLSGKPGVDDSWPTSDDEDLNEDTKNVPKPSLAKLMTASQQSRKNLEA | |
| | TYGTVRTGNRTLFEDRDSDSQDEVVVESLPTTSIKVQCFSHPTYQSPDL | |
| | LPKPSHKSLANPGLMKEEPTKPGIAKKENGIDIIESAPLEQTNNDNLTY | |
| | VDEVHKNNRSDMMSALGLGQEEDIESPWDSESISENFPQKYVDPLAGAA | |
| | DGKEKNIGNEQAEDVFYIPSCMSGSRNFKMAKLEDTRNVGMPVAHMESP | |
| | ERYLHLKPTIEMKDSVPNKAGGMKDVQTSKAAEHDLEVASEEEQEREGS | |
| | ENNQPQVEEERKKHRNNEMEVSANIHDGATDDAEDDDDDDGLIQKRKSG | |
| | ETDHQQFPRKENKEYASSGPALQMKEVKSTEKEKRTSKESVNSPVFGKA | |
| | SLLTGGLLQVDDDSSLSEIDEDEGRPTKKTSNEKNKVKNQIQSMDDVDD | |
| | LTQSSETASEDCELPHSSYKNEMLLIEQLGMECKDSVSLLKIQDAALSC | |
| | ERLLELKKNHCELLTVKIKKMEDKVNVLQRELSETKEIKSQLEHQKVEW | |
| | ERELCSLRFSLNQEEEKRRNADTLYEKIREQLRRKEEQYRKEVEVKQQL | |
| | ELSLQTLEMELRTVKSNLNQVVQERNDAQRQLSREQNARMLQDGILTNH | |
| | LSKQKEIEMAQKKMNSENSHSHEEEKDLSHKNSMLQEEIAMLRLEIDTI | |
| | KNQNQEKEKKCFEDLKIVKEKNEDLQKTIKQNEETLTQTISQYNGRLSV | |
| | LTAENAMLNSKLENEKQSKERLEAEVESYHSRLAAAIHDRDQSETSKRE | |
| | LELAFQRARDECSRLQDKMNFDVSNLKDNNEILSQQLFKTESKLNSLEI | |
| | EFHHTRDALREKTLGLERVQKDLSQTQCQMKEMEQKYQNEQVKVNKYIG | |
| | KQESVEERLSQLQSENMLLRQQLDDAHNKADNKEKTVINIQDQFHAIVQ | |
| | KLQAESEKQSLLLEERNKELISECNHLKERQYQYENEKAEREVVVRQLQ | |
| | QELADTLKKQSMSEASLEVTSRYRINLEDETQDLKKKLGQIRNQLQEAQ | |
| | DRHTEAVRCAEKMQDHKQKLEKDNAKLKVTVKKQMDKIEELQKNLLNAN | |
| | LSEDEKEQLKKLMELKQSLECNLDQEMKKNVELEREITGFKNLLKMTRK | |
| | KLNEYENGEFSFHGDLKTSQFEMDIQINKLKHKEDPKWEFPRKNLVLGK | |
| | TLGEGEFGKVVKATAFHLKGRAGYTTVAVKMLKENASPSELRDLLSEEN | |
| | VLKQVNHPHVIKLYGACSQDGPLLLIVEYAKYGSLRGELRESRKVGPGY | |
| | LGSGGSRNSSSLDHPDERALTMGDLISFAWQISQGMQYLAEMKLVHRDL | |
| | AARNILVAEGRKMKISDEGLSRDVYEEDSYVKRSQGRIPVKWMAIESLE | |
| | DHIYTTQSDVWSFGVLLWEIVTLGGNPYPGIPPERLENLLKTGHRMERP | |
| | DNCSEEMYRLMLQCWKQEPDKRPVFADISKDLEKMMVKRRDYLDLAAST | |
| | PSDSLIYDDGLSEEETPLVDCNNAPLPRALPSTWIENKLYGRISHAFTR | |
| | F | |
| CLIP1-RET | MSMLKPSGLKAPTKILKPGSTALKTPTAVVAPVEKTISSEKASSTPSSE | 106 |
| | TQEEFVDDERVGERVWVNGNKPGFIQFLGETQFAPGQWAGIVLDEPIGK | |
| | NDGSVAGVRYFQCEPLKGIFTRPSKLTRKVQAEDEANGLQTTPASRATS | |
| | PLCTSTASMVSSSPSTPSNIPQKPSQPAAKEPSATPPISNLTKTASESI | |
| | SNLSEAGSIKKGERELKIGDRVLVGGTKAGVVRELGETDFAKGEWCGVE | |
| | LDEPLGKNDGAVAGTRYFQCQPKYGLFAPVHKVTKIGFPSTTPAKAKAN | |
| | AVRRVMATTSASLKRSPSASSLSSMSSVASSVSSRPSRTGLLTETSSRY | |
| | ARKISGTTALQEALKEKQQHIEQLLAERDLERAEVAKATSHVGEIEQEL | |
| | ALARDGHDQHVLELEAKMDQLRTMVEAADREKVELLNQLEEEKRKVEDL | |
| | QFRVEEESITKGDLETQTKLEHARIKELEQSLLFEKTKADKLQRELEDT | |
| | RVATVSEKSRIMELEKDLALRVQEVAELRRRLESNKPAGDVDMSLSLLQ | 107 |
| | EISSLQEKLEVTRTDHQREITSLKEHFGAREETHQKEIKALYTATEKLS | |
| | KENESLKSKLEHANKENSDVIALWKSKLETAIASHQQAMEELKVSESKG | |
| | LGTETAEFAELKTQIEKMRLDYQHEIENLQNQQDSERAAHAKEMEALRA | |
| | KLMKVIKEKENSLEAIRSKLDKAEDQHLVEMEDTLNKLQEAEIKVKELE | |
| | VLQAKCNEQTKVIDNFTSQLKATEEKLLDLDALRKASSEGKSEMKKLRQ | |
| | QLEAAEKQIKHLEIEKNAESSKASSITRELQGRELKLTNLQENLSEVSQ | |
| | VKETLEKELQILKEKFAEASEEAVSVQRSMQETVNKLHQKEEQFNMLSS | |
| | DLEKLRENLADMEAKFREKDEREEQLIKAKEKLENDIAEIMKMSGDNSS | |
| | QLTKMNDELRLKERDVEELQLKLTKANENASFLQKSIEDMTVKAEQSQQ | |
| | EAAKKHEEEKKELERKLSDLEKKMETSHNQCQELKARYERATSETKTKH | |
| | EEILQNLQKTLLDTEDKLKGAREENSGLLQELEELRKQADKAKEDPKWE | |
| | FPRKNLVLGKTLGEGEFGKVVKATAFHLKGRAGYTTVAVKMLKENASPS | |
| | ELRDLLSEFNVLKQVNHPHVIKLYGACSQDGPLLLIVEYAKYGSLRGEL | |
| | RESRKVGPGYLGSGGSRNSSSLDHPDERALTMGDLISFAWQISQGMQYL | |
| | AEMKLVHRDLAARNILVAEGRKMKISDFGLSRDVYEEDSYVKRSQGRIP | |
| | VKWMAIESLFDHIYTTQSDVWSFGVLLWEIVTLGGNPYPGIPPERLENL | |
| | LKTGHRMERPDNCSEEMYRLMLQCWKQEPDKRPVFADISKDLEKMMVKR | |
| | RDYLDLAASTPSDSLIYDDGLSEEETPLVDCNNAPLPRALPSTWIENKL | |
| | YGRISHAFTRF | |
| DLG5-RET | MEPQRRELLAQCQQSLAQAMTEVEAVLGLLEAAGALSPGERRQLDEEAG | |
| | GAKAELLLKLLLAKERDHFQDLRAALEKTQPHLLPILYLNGVVGPPQPA | |
| | EGAGSTYSVLSTMPSDSESSSSLSSVGTTGKAPSPPPLLTDQQVNEKVE | |
| | NLSIQLRLMTRERNELRKRLAFATHGTAFDKRPYHRLNPDYERLKIQCV | |
| | RAMSDLQSLQNQHTNALKRCEEVAKETDFYHTLHSRLLSDQTRLKDDVD | |
| | MLRRENGQLLRERNLLQQSWEDMKRLHEEDQKEIGDLRAQQQQVLKHNG | |
| | SSEILNKLYDTAMDKLEVVKKDYDALRKRYSEKVAIHNADLSRLEQLGE | |
| | ENQRLLKQTEMLTQQRDTAIQLQHQCALSLRRFEAIHHELNKATAQNKD | |
| | LQWEMELLQSELTELRTTQVKTAKESEKYREERDAVYSEYKLIMSERDQ | |
| | VISELDKLQTEVELAESKLKSSTSEKKAANEEMEALRQIKDTVTMDAGR | |
| | ANKEVEILRKQCKALCQELKEALQEADVAKCRRDWAFQERDKIVAERDS | |
| | IRTLCDNLRRERDRAVSELAEALRSLDDTRKQKNDVSRELKELKEQMES | |
| | QLEKEARFRQLMAHSSHDSAIDTDSMEWETEVVEFERETEDIDLKALGE | |
| | DMAEGVNEPCFPGDCGIFVTKVDKGSIADGRLRVNDWLLRINDVDLINK | |
| | DKKQAIKALLNGEGAINMVVRRRKSLGGKVVTPLHINLSGQKDSGISLE | |
| | NGVYAAAVLPGSPAAKEGSLAVGDRIVAINGIALDNKSLNECESLLRSC | |
| | QDSLTLSLLKEDPKWEFPRKNLVLGKTLGEGEFGKVVKATAFHLKGRAG | |
| | YTTVAVKMLKENASPSELRDLLSEFNVLKQVNHPHVIKLYGACSQDGPL | |
| | LLIVEYAKYGSLRGFLRESRKVGPGYLGSGGSRNSSSLDHPDERALTMG | |
| | DLISFAWQISQGMQYLAEMKLVHRDLAARNILVAEGRKMKISDEGLSRD | |
| | VYEEDSYVKRSQGRIPVKWMAIESLFDHIYTTQSDVWSFGVLLWEIVTL | |
| | GGNPYPGIPPERLENLLKTGHRMERPDNCSEEMYRLMLQCWKQEPDKRP | |
| | VFADISKDLEKMMVKRRDYLDLAASTPSDSLIYDDGLSEEETPLVDCNN | |
| | APLPRALPSTWIENKLYGRISHAFTRF | |
| DLG5-RET | MEPQRRELLAQCQQSLAQAMTEVEAVLGLLEAAGALSPGERRQLDEEAG | 108 |
| | GAKAELLLKLLLAKERDHFQDLRAALEKTQPHLLPILYLNGVVGPPQPA | |
| | EGAGSTYSVLSTMPSDSESSSSLSSVGTTGKAPSPPPLLTDQQVNEKVE | |
| | NLSIQLRLMTRERNELRKRLAFATHGTAFDKRPYHRLNPDYERLKIQCV | |
| | RAMSDLQSLQNQHTNALKRCEEVAKETDFYHTLHSRLLSDQTRLKDDVD | |
| | MLRRENGQLLRERNLLQQSWEDMKRLHEEDQKEIGDLRAQQQQVLKHNG | |
| | SSEILNKLYDTAMDKLEVVKKDYDALRKRYSEKVAIHNADLSRLEQLGE | |
| | ENQRLLKQTEMLTQQRDTAIQLQHQCALSLRRFEAIHHELNKATAQNKD | |
| | LQWEMELLQSELTELRTTQVKTAKESEKYREERDAVYSEYKLIMSERDQ | |
| | VISELDKLQTEVELAESKLKSSTSEKKAANEEMEALRQIKDTVTMDAGR | |
| | ANKEVEILRKQCKALCQELKEALQEADVAKCRRDWAFQERDKIVAERDS | |
| | IRTLCDNLRRERDRAVSELAEALRSLDDTRKQKNDVSRELKELKEQMES | |
| | QLEKEARFRQLMAHSSHDSAIDTDSMEWETEVVEFERETEDIDLKALGF | |
| | DMAEGVNEPCFPGDCGIFVTKVDKGSIADGRLRVNDWLLRINDVDLINK | |
| | DKKQAIKALLNGEGAINMVVRRRKSLGGKVVTPLHINLSGQKDSGISLE | |
| | NGVYAAAVLPGSPAAKEGSLAVGDRIVAINGIALDNKSLNECESLLRSC | |
| | QDSLTLSLLKVFPQSSSWSGQNIFENIKDSDKMLSFRAHGPEVQAHNKR | |
| | NLIQHNNSTQTDIFYTDRLEDRKEPGPPGGSSSELHKPFPGGPLQVCPQ | |
| | ACPSASERSLSSFRSDASGDRGFGLVDVRGRRPLLPFETEVGPCGVGEA | |
| | SLDKADSEGSNSGGTWPKAMLSSTAVPEKLSVYKKPKQRKSIFDPNTEK | |
| | RPQTPPKIDYLLPGPGPAHSPQPSKRAGPLTPPKPPRRSDSIKFQHRLE | |
| | TSSESEATLVGSSPSTSPPSALPPDVDPGEPMHASPPRKARVRIASSYY | |
| | PEGDGDSSHLPAKKSCDEDLTSQKVDELGQKRRRPKSAPSFRPKLAPVV | |
| | IPAQFLEEQKCVPASGELSPELQEWAPYSPGHSSRHSNPPLYPSRPSVG | |
| | TVPRSLTPSTTVSSILRNPIYTVRSHRVGPCSSPPAARDAGPQGLHPSV | |
| | QHQGRLSLDLSHRTCSDYSEMRATHGSNSLPSSARLGSSSNLQFKAERI | |
| | KIPSTPRYPRSVVGSERGSVSHSECSTPPQSPLNIDTLSSCSQSQTSAS | |
| | TLPRIAVNPASLGERRKDRPYVEEPRHVKVQKGSEPLGISIVSGEKGGI | |
| | YVSKVTVGSIAHQAGLEYGDQLLEENGINLRSATEQQARLIIGQQCDTI | |
| | TILAQYNPHVHQLSSHSRSSSHLDPAGTHSTLQGSGTTTPEHPSVIDPL | |
| | MEQDEGPSTPPAKQSSSRIAGDANKKTLEPRVVFIKKSQLELGVHLCGG | |
| | NLHGVFVAEVEDDSPAKGPDGLVPGDLILEYGSLDVRNKTVEEVYVEML | |
| | KPRDGVRLKVQYRPEEFTKAKGLPGDSFYIRALYDRLADVEQELSEKKD | |
| | DILYVDDTLPQGTFGSWMAWQLDENAQKIQRGQIPSKYVMDQEFSRRLS | |
| | MSEVKDDNSATKTLSAAARRSFFRRKHKHKRSGSKDGKDLLALDAFSSD | |
| | SIPLFEDVAEEAGCPLSCAVSKRRLECEECGGLGSPTGRCEWRQGDGKG | |
| | ITRNESTCSPSTKTCPDGHCDVVETQDINICPQDCLRGSIVGGHEPGEP | |
| | RGIKAGYGTCNCFPEEEKCFCEPEDIQDPLCDELCRTVIAAAVLESFIV | |
| | SVLLSAFCIHCYHKFAHKPPISSAEMTERRPAQAFPVSYSSSGARRPSL | |
| | DSMENQVSVDAFKILEDPKWEFPRKNLVLGKTLGEGEFGKVVKATAFHL | |
| | KGRAGYTTVAVKMLKENASPSELRDLLSEFNVLKQVNHPHVIKLYGACS | |
| | QDGPLLLIVEYAKYGSLRGELRESRKVGPGYLGSGGSRNSSSLDHPDER | |
| | ALTMGDLISFAWQISQGMQYLAEMKLVHRDLAARNILVAEGRKMKISDE | |
| | GLSRDVYEEDSYVKRSQGRIPVKWMAIESLFDHIYTTQSDVWSFGVLLW | |
| | EIVTLGGNPYPGIPPERLENLLKTGHRMERPDNCSEEMYRLMLQCWKQE | |
| | PDKRPVFADISKDLEKMMVKRRDYLDLAASTPSDSLIYDDGLSEEETPL | |
| | VDCNNAPLPRALPSTWIENKLYGRISHAFTRE | |
| ERC1-RET | MYGSARSVGKVEPSSQSPGRSPRLPRSPRLGHRRTNSTGGSSGSSVGGG | 109 |
| | SGKTLSMENIQSLNAAYATSGPMYLSDHENVGSETPKSTMTLGRSGGRL | |
| | PYGVRMTAMGSSPNIASSGVASDTIAFGEHHLPPVSMASTVPHSLRQAR | |
| | DNTIMDLQTQLKEVLRENDLLRKDVEVKESKLSSSMNSIKTFWSPELKK | |
| | ERALRKDEASKITIWKEQYRVVQEENQHMQMTIQALQDELRIQRDLNQL | |
| | FQQDSSSRTGEPCVAELTEENFQRLHAEHERQAKELELLRKTLEEMELR | |
| | IETQKQTLNARDESIKKLLEMLQSKGLSAKATEEDHERTRRLAEAEMHV | |
| | HHLESLLEQKEKENSMLREEMHRRFENAPDSAKTKALQTVIEMKDSKIS | |
| | SMERGLRDLEEEIQMLKSNGALSTEEREEEMKQMEVYRSHSKEMKNKIG | |
| | QVKQELSRKDTELLALQTKLETLTNQFSDSKQHIEVLKESLTAKEQRAA | |
| | ILQTEVDALRLRLEEKETMLNKKTKQIQDMAEEKGTQAGEIHDLKDMLD | |
| | VKERKVNVLQKKIENLQEQLRDKEKQMSSLKERVKSLQADTTNTDTALT | |
| | TLEEALAEKERTIERLKEQRDRDEREKQEEIDNYKKDLKDLKEKVSLLQ | |
| | GDLSEKEASLLDLKEHASSLASSGLKKDSRLKTLEIALEQKKEECLKME | |
| | SQLKKAHEAALEARASPEMSDRIQHLEREITRYKDESSKAQAEVDRLLE | |
| | ILKEVENEKNDKDKKIAELERQVKDQNKKVANLKHKEQVEKKKSAQMLE | |
| | EARRREDNLNDSSQQLQEDPKWEFPRKNLVLGKTLGEGEFGKVVKATAF | |
| | HLKGRAGYTTVAVKMLKENASPSELRDLLSEENVLKQVNHPHVIKLYGA | |
| | CSQDGPLLLIVEYAKYGSLRGFLRESRKVGPGYLGSGGSRNSSSLDHPD | |
| | ERALTMGDLISFAWQISQGMQYLAEMKLVHRDLAARNILVAEGRKMKIS | |
| | DFGLSRDVYEEDSYVKRSQGRIPVKWMAIESLFDHIYTTQSDVWSFGVL | |
| | LWEIVTLGGNPYPGIPPERLENLLKTGHRMERPDNCSEEMYRLMLQCWK | |
| | QEPDKRPVFADISKDLEKMMVKRRDYLDLAASTPSDSLIYDDGLSEEET | |
| | PLVDCNNAPLPRALPSTWIENKLYGRISHAFTRE | |
| ERC1-RET | MYGSARSVGKVEPSSQSPGRSPRLPRSPRLGHRRTNSTGGSSGSSVGGG | 110 |
| | SGKTLSMENIQSLNAAYATSGPMYLSDHENVGSETPKSTMTLGRSGGRL | |
| | PYGVRMTAMGSSPNIASSGVASDTIAFGEHHLPPVSMASTVPHSLRQAR | |
| | DNTIMDLQTQLKEVLRENDLLRKDVEVKESKLSSSMNSIKTFWSPELKK | |
| | ERALRKDEASKITIWKEQYRVVQEENQHMQMTIQALQDELRIQRDLNQL | |
| | FQQDSSSRTGEPCVAELTEENFQRLHAEHERQAKELFLLRKTLEEMELR | |
| | IETQKQTLNARDESIKKLLEMLQSKGLSAKATEEDHERTRRLAEAEMHV | |
| | HHLESLLEQKEKENSMLREEMHRRFENAPDSAKTKALQTVIEMKDSKIS | |
| | SMERGLRDLEEEIQMLKSNGALSTEEREEEMKQMEVYRSHSKEMKNKIG | |
| | QVKQELSRKDTELLALQTKLETLTNQFSDSKQHIEVLKESLTAKEQRAA | |
| | ILQTEVDALRLRLEEKETMLNKKTKQIQDMAEEKGTQAGEIHDLKDMLD | |
| | VKERKVNVLQKKIENLQEQLRDKEKQMSSLKERVKSLQADTTNTDTALT | |
| | TLEEALAEKEDPKWEFPRKNLVLGKTLGEGEFGKVVKATAFHLKGRAGY | |
| | TTVAVKMLKENASPSELRDLLSEFNVLKQVNHPHVIKLYGACSQDGPLL | |
| | LIVEYAKYGSLRGFLRESRKVGPGYLGSGGSRNSSSLDHPDERALTMGD | |
| | LISFAWQISQGMQYLAEMKLVHRDLAARNILVAEGRKMKISDFGLSRDV | |
| | YEEDSYVKRSQGRIPVKWMAIESLEDHIYTTQSDVWSFGVLLWEIVTLG | |
| | GNPYPGIPPERLENLLKTGHRMERPDNCSEEMYRLMLQCWKQEPDKRPV | |
| | FADISKDLEKMMVKRRDYLDLAASTPSDSLIYDDGLSEEETPLVDCNNA | |
| | PLPRALPSTWIENKLYGRISHAFTRE | |
| ERC1-RET | MYGSARSVGKVEPSSQSPGRSPRLPRSPRLGHRRTNSTGGSSGSSVGGG | 111 |
| | SGKTLSMENIQSLNAAYATSGPMYLSDHENVGSETPKSTMTLGRSGGRL | |
| | PYGVRMTAMGSSPNIASSGVASDTIAFGEHHLPPVSMASTVPHSLRQAR | |
| | DNTIMDLQTQLKEVLRENDLLRKDVEVKESKLSSSMNSIKTFWSPELKK | |
| | ERALRKDEASKITIWKEQYRVVQEENQHMQMTIQALQDELRIQRDLNQL | |
| | FQQDSSSRTGEPCVAELTEENFQRLHAEHERQAKELFLLRKTLEEMELR | |
| | IETQKQTLNARDESIKKLLEMLQSKGLSAKATEEDHERTRRLAEAEMHV | |
| | HHLESLLEQKEKENSMLREEMHRRFENAPDSAKTKALQTVIEMKDSKIS | |
| | SMERGLRDLEEEIQMLKSNGALSTEEREEEMKQMEVYRSHSKEMKNKIG | |
| | QVKQELSRKDTELLALQTKLETLTNQFSDSKQHIEVLKESLTAKEQRAA | |
| | ILQTEVDALRLRLEEKETMLNKKTKQIQDMAEEKGTQAGEIHDLKDMLD | |
| | VKERKVNVLQKKIENLQEQLRDKEKQMSSLKERVKSLQADTTNTDTALT | |
| | TLEEALAEKERTIERLKEQRDRDEREKQEEIDNYKKDLKDLKEKVSLLQ | |
| | GDLSEKEASLLDLKEHASSLASSGLKKDSRLKTLEIALEQKKEECLKME | |
| | SQLKKAHEAALEARASPEMSDRIQHLEREITRYKDESSKAQAEVDRLLE | |
| | ILKEVENEKNDKDKKIAELERQVKDQNKKVANLKHKEQVEKKKSAQMLE | |
| | EARRREDNLNDSSQQLQIHCATSCAARSQPLSSSPSSSRCCCLPSASTA | |
| | TTSLPTSHPSPQLRPSGGPPRPSRSATPLPVPAGPRWTPWRTRSPWMPS | |
| | RSWRIQSGNSLGRTWFLEKLEKANLEKWSRQRPSIKAEQGTPRWPRCKR | |
| | TPPRVSCETCCQSSTSSRSTTHMSSNCMGPAARMARSSSSWSTPNTAPC | |
| | GASSARAAKWGLATWAVEAAATPAPWTTRMSGPSPWATSSHLPGRSHRG | |
| | CSIWPRSSFIGTWQPETSWLRGGRRFRISACPEMEMKRIPTRGARVGFQ | |
| | LNGWQLNPFLIISTPRKVMYGLLVSCCGRSPGETPILGFLLSGSSTERP | |
| | ATGWRGQTTAARRCTACCNAGSRSRTKGRCLRTSAKTWRRWLRGETTWT | |
| | LRRPLHLTPFMTTASQRRRHRWWTVIMPPSLEPSLPHGLKTNSMVEFPM | |
| | HLLDS | |
| FRMD4A- | MAVQLVPDSALGLLMMTEGRRCQVHLLDDRKLELLVQPKLLAKELLDLV | 112 |
| RET | ASHFNLKEKEYFGIAFTDETGHLNWLQLDRRVLEHDFPKKSGPVVLYFC | |
| | VRFYIESISYLKDNATIELFFLNAKSCIYKELIDVDSEVVFELASYILQ | |
| | EAKGDFSSNEVVRSDLKKLPALPTQALKEHPSLAYCEDRVIEHYKKLNG | |
| | QTRGQAIVNYMSIVESLPTYGVHYYAVKDKQGIPWWLGLSYKGIFQYDY | |
| | HDKVKPRKEDPKWEFPRKNLVLGKTLGEGEFGKVVKATAFHLKGRAGYT | |
| | TVAVKMLKENASPSELRDLLSEFNVLKQVNHPHVIKLYGACSQDGPLLL | |
| | IVEYAKYGSLRGELRESRKVGPGYLGSGGSRNSSSLDHPDERALTMGDL | |
| | ISFAWQISQGMQYLAEMKLVHRDLAARNILVAEGRKMKISDFGLSRDVY | |
| | EEDSYVKRSQGRIPVKWMAIESLEDHIYTTQSDVWSFGVLLWEIVTLGG | |
| | NPYPGIPPERLENLLKTGHRMERPDNCSEEMYRLMLQCWKQEPDKRPVF | |
| | ADISKDLEKMMVKRRDYLDLAASTPSDSLIYDDGLSEEETPLVDCNNAP | |
| | LPRALPSTWIENKLYGRISHAFTRF | |
| KIAA1468 | MAAMAPGGSGSGGGVNPFLSDSDEDDDEVAATEERRAVLRLGAGSGLDP | 113 |
| RET | GSAGSLSPQDPVALGSSARPGLPGEASAAAVALGGTGETPARLSIDAIA | |
| | AQLLRDQYLLTALELHTELLESGRELPRLRDYFSNPGNFERQSGTPPGM | |
| | GAPGVPGAAGVGGAGGREPSTASGGGQLNRAGSISTLDSLDFARYSDDG | |
| | NRETDEKVAVLEFELRKAKETIQALRANLTKAAEHEVPLQERKNYKSSP | |
| | EIQEPIKPLEKRALNFLVNEFLLKNNYKLTSITESDENDDQDFELWDDV | |
| | GLNIPKPPDLLQLYRDFGNHQVTGKDLVDVASGVEEDELEALTPIISNL | |
| | PPTLETPQPAENSMLVQKLEDKISLLNSEKWSLMEQIRRLKSEMDELKN | |
| | EHFAIPAVCDSVQPPLDQLPHKDSEDSGQHPDVNSSDKGKNTDIHLSIS | |
| | DEADSTIPKENSPNSFPRREREGMPPSSLSSKKTVHEDKPNRKLSPAFH | |
| | QALLSFCRMSADSRLGYEVSRIADSEKSVMLMLGRCLPHIVPNVLLAKR | |
| | EEDPKWEFPRKNLVLGKTLGEGEFGKVVKATAFHLKGRAGYTTVAVKML | |
| | KENASPSELRDLLSEFNVLKQVNHPHVIKLYGACSQDGPLLLIVEYAKY | |
| | GSLRGFLRESRKVGPGYLGSGGSRNSSSLDHPDERALTMGDLISFAWQI | |
| | SQGMQYLAEMKLVHRDLAARNILVAEGRKMKISDEGLSRDVYEEDSYVK | |
| | RSQGRIPVKWMAIESLEDHIYTTQSDVWSFGVLLWEIVTLGGNPYPGIP | |
| | PERLENLLKTGHRMERPDNCSEEMYRLMLQCWKQEPDKRPVFADISKDL | |
| | EKMMVKRRDYLDLAASTPSDSLIYDDGLSEEETPLVDCNNAPLPRALPS | |
| | TWIENKLYGRISHAFTRE | |
| NCOA4-RET | MNTFQDQSGSSSNREPLLRCSDARRDLELAIGGVLRAEQQIKDNLREVK | 114 |
| | AQIHSCISRHLECLRSREVWLYEQVDLIYQLKEETLQQQAQQLYSLLGQ | |
| | FNCLTHQLECTQNKDLANQVSVCLERLGSLTLKPEDSTVLLFEADTITL | |
| | RQTITTFGSLKTIQIPEHLMAHASSANIGPFLEKRGCISMPEQKSASGI | |
| | VAVPFSEWLLGSKPASGYQAPYIPSTDPQDWLTQKQTLENSQTSSRACN | |
| | FFNNVGGNLKGLENWLLKSEKSSYQKCNSHSTTSSFSIEMEKVGDQELP | |
| | DQDEMDLSDWLVTPQESHKLRKPENGSRETSEKFKLLFQSYNVNDWLVK | |
| | TDSCTNCQGNQPKGVEIENLGNLKCLNDHLEAKKPLSTPSMVTEDWLVQ | |
| | NHQDPCKVEEVCRANEPCTSFAECVCDENCEKEALYKWLLKKEGKDKNG | |
| | MPVEPKPEPEKHKDSLNMWLCPRKEVIEQTKAPKAMTPSRIADSFQVIK | |
| | NSPLSEWLIRPPYKEGSPKEVPGTEDRAGKQKFKSPMNTSWCSENTADW | |
| | VLPGKKMGNLSQLSSGEDKWLLRKKAQEDPKWEFPRKNLVLGKTLGEGE | |
| | FGKVVKATAFHLKGRAGYTTVAVKMLKENASPSELRDLLSEENVLKQVN | |
| | HPHVIKLYGACSQDGPLLLIVEYAKYGSLRGFLRESRKVGPGYLGSGGS | |
| | RNSSSLDHPDERALTMGDLISFAWQISQGMQYLAEMKLVHRDLAARNIL | |
| | VAEGRKMKISDEGLSRDVYEEDSYVKRSQGRIPVKWMAIESLEDHIYTT | |
| | QSDVWSFGVLLWEIVTLGGNPYPGIPPERLENLLKTGHRMERPDNCSEE | |
| | MYRLMLQCWKQEPDKRPVFADISKDLEKMMVKRRDYLDLAASTPSDSLI | |
| | YDDGLSEEETPLVDCNNAPLPRALPSTWIENKLYGRISHAFTRE | |
| PARD3-RET | MKVTVCFGRTRVVVPCGDGHMKVFSLIQQAVTRYRKAIAKDPNYWIQVH | 115 |
| | RLEHGDGGILDLDDILCDVADDKDRMWPRRRAAPCPVQSARDGWSVRSV | |
| | AAWAPQQAGVSGGKEMAKGSPGTSPPALPAPRPAPTATAMLWRPKTSTE | |
| | ALRTASGAALLGDTSLGSPGGLKLAMAPATASLRRRSASASPKTSRIHC | |
| | ATSCAARSQPLSSSPSSSRCCCLPSASTATTSLPTSHPSPQLRPSGGPP | |
| | RPSRSATPLPVPAGPRWTPWRTRSPWMPSRSWRIQSGNSLGRTWFLEKL | |
| | EKANLEKWSRQRPSIKAEQGTPRWPRCKRTPPRVSCETCCQSSTSSRST | |
| | THMSSNCMGPAARMARSSSSWSTPNTAPCGASSARAAKWGLATWAVEAA | |
| | ATPAPWTTRMSGPSPWATSSHLPGRSHRGCSIWPRSSFIGTWQPETSWL | |
| | RGGRRFRISACPEMEMKRIPTRGARVGFQLNGWQLNPFLIISTPRKVMY | |
| | GLLVSCCGRSPGETPILGFLLSGSSTFRPATGWRGQTTAARRCTACCNA | |
| | GSRSRTKGRCLRTSAKTWRRWLRGETTWTLRRPLHLTPFMTTASQRRRH | |
| | RWWTVIMPPSLEPSLPHGLKTNSMVEFPMHLLDS | |
| PARD3-RET | MKVTVCFGRTRVVVPCGDGHMKVFSLIQQAVTRYRKAIAKDPNYWIQVH | 116 |
| | RLEHGDGGILDLDDILCDVADDKDRLVAVEDEQDPHHGGDGTSASSTGT | |
| | QSPEIFGSELGTNNVSAFQPYQATSEIEVTPSVLRANMPLHVRRSSDPA | |
| | LIGLSTSVSDSNESSEEPSRKNPTRWSTTAGELKQNTAGSPKTCDRKED | |
| | PKWEFPRKNLVLGKTLGEGEFGKVVKATAFHLKGRAGYTTVAVKMLKEN | |
| | ASPSELRDLLSEENVLKQVNHPHVIKLYGACSQDGPLLLIVEYAKYGSL | |
| | RGFLRESRKVGPGYLGSGGSRNSSSLDHPDERALTMGDLISFAWQISQG | |
| | MQYLAEMKLVHRDLAARNILVAEGRKMKISDFGLSRDVYEEDSYVKRSQ | |
| | GRIPVKWMAIESLFDHIYTTQSDVWSFGVLLWEIVTLGGNPYPGIPPER | |
| | LFNLLKTGHRMERPDNCSEEMYRLMLQCWKQEPDKRPVFADISKDLEKM | |
| | MVKRRDYLDLAASTPSDSLIYDDGLSEEETPLVDCNNAPLPRALPSTWI | |
| | ENKLYGRISHAFTRE | |
| PRKAR1A- | MESGSTAASEEARSLRECELYVQKHNIQALLKDSIVQLCTARPERPMAF | 117 |
| RET | LREYFERLEKEEAKQIQNLQKAGTRTDSREDEISPPPPNPVVKGRRRRG | |
| | AISAEVYTEEDAASYVRKVIPKDYKTMAALAKAIEKNVLFSHLDDNERS | |
| | DIFDAMFSVSFIAGETVIQQGDEGDNFYVIDQGETDVYVNNEWATSVGE | |
| | GGSFGELALIYGTPRAATVKAKTNVKLWGIDRDSYRRILMEDPKWEFPR | |
| | KNLVLGKTLGEGEFGKVVKATAFHLKGRAGYTTVAVKMLKENASPSELR | |
| | DLLSEFNVLKQVNHPHVIKLYGACSQDGPLLLIVEYAKYGSLRGELRES | |
| | RKVGPGYLGSGGSRNSSSLDHPDERALTMGDLISFAWQISQGMQYLAEM | |
| | KLVHRDLAARNILVAEGRKMKISDFGLSRDVYEEDSYVKRSQGRIPVKW | |
| | MAIESLEDHIYTTQSDVWSFGVLLWEIVTLGGNPYPGIPPERLENLLKT | |
| | GHRMERPDNCSEEMYRLMLQCWKQEPDKRPVFADISKDLEKMMVKRRDY | |
| | LDLAASTPSDSLIYDDGLSEEETPLVDCNNAPLPRALPSTWIENKLYGR | |
| | ISHAFTRF | |
| PRKG1-RET | MGTLRDLQYALQEKIEELRQRDALIDELELELDQKDELIQKLQNELDKY | 118 |
| | RSVIRPATQQAQKQSASTLQGEPRTKRQAISAEPTAFDIQDLSHVTLPE | |
| | YPKSPQSKDLIKEAILDNDEMKNLELSQIQEIVDCMYPVEYGKDSCIIK | |
| | EGDVGSLVYVMEDGKVEVTKEGVKLCTMGPGKVFGELAILYNCTRTATV | |
| | KTLVNVKLWAIDRQCFQTIMMRTGLIKHTEYMEFLKSVPTFQSLPEEIL | |
| | SKLADVLEETHYENGEYIIRQGARGDTFFIISKGTVNVTREDSPSEDPV | |
| | FLRTLGKGDWFGEKALQGGSALPLRPGQPGGEHALGPGPRAAGEVRAGG | |
| | RVHRARRRARGGGDGALPGDRVRRGRLGAHLPRGRRHRQRRGGVQAEGG | |
| | HRGGHAACLRCRRGTCIRGAGEAVHKHAAPRGHLGPADLPGGTLAQRDL | |
| | GPGQRQLRAGDRTLAGSQPEPLHLGEPHHAAGGAGQLRLPGPRSGRPLA | |
| | PLQRVGAAGQPAPAQYLLPLREQEGSPICPDRESLCGKLPGIQWHQRPV | |
| | QAAFLWCQLQHARGGHLSRGHLGDPVCEHQGPAAAQVCRTSLHGGGHRP | |
| | ADLAGPGPAACNSGGVICGRGGGLPPVLCSQQETAGVGVWRPGLPNRQV | |
| | VEARRWQRDHQELLHLLSQHQDLPRRPLRCCGDPRHQHLPSGLPPGQHC | |
| | WGTRAWGAPGDSWLWHLQLLPGGEVLLRARRHPGSTVRRAVPHGDRSRC | |
| | PLLLHRLGAAVCLLHPLLPQVCPQATHLLSDDLPEARPGLPGQLLLERC | |
| | PPALAGLHGEPGLRGCLQDPGGSKVGIPSEELGSWKNSRRRRIWKSGQG | |
| | NGLPSERQSRVHHGGREDAERERLPEAARPAVRVQRPEAGQPPTCHQIV | |
| | WGLQPGWPAPPHRGVRQIRLPAGLPPREPQSGAWLPGQWRQPQLQLPGP | |
| | PGAGPHHGRPHLICLADLTGDAVSGRDEARSSGLGSQKHPGSGAEDEDE | |
| | GFRLVPRCLRGELREEEPGSDSSMDGNIPFSYLHHAKCMVEWCPAVGDR | |
| | DPRGKPLSWDSSAALQPSEDRPPDGEARQLQRGDVPPDAAMLEAGAGQK | |
| | AGVCGHQQRPGEDDGEERLLGPCGVHSILPDLRRPLRGGDTAGGLCPPP | |
| | SSPPFHMDKQTLWNFPCIYIL | |
| PRKG1-RET | MGTLRDLQYALQEKIEELRQRDALIDELELELDQKDELIQKLQNELDKY | 119 |
| | RSVIRPATQQAQKQSASTLQGEPRTKRQAISAEPTAFDIQDLSHVTLPF | |
| | YPKSPQSKDLIKEAILDNDEMKNLELSQIQEIVDCMYPVEYGKDSCIIK | |
| | EGDVGSLVYVMEDGKVEVTKEGVKLCTMGPGKVFGELAILYNCTRTATV | |
| | KTLVNVKLWAIDRQCFQTIMMRTGLIKHTEYMEFLKSVPTFQSLPEEIL | |
| | SKLADVLEEVPPDAAMLEAGAGQKAGVCGHQQRPGEDDGEERLLGPCGV | |
| | HSILPDLRRPLRGGDTAGGLCPPPSSPPFHMDKQTLWNFPCIYIL | |
| RUFY2-RET | MTFQVWGWRREDASQVLAWVPDAEGGRRGMLTRRSLATKDPTAVERANL | 120 |
| | LNMAKLSIKGLIESALSFGRTLDSDYPPLQQFFVVMEHCLKHGLKVRKS | |
| | FLSYNKTIWGPLELVEKLYPEAEEIGASVRDLPGLKTPLGRARAWLRLA | |
| | LMQKKMADYLRCLIIQRDLLSEFYEYHALMMEEEGAVIVGLLVGLNVID | |
| | ANLCVKGEDLDSQVGVIDESMYLKNEEDIGNKERNVQIAAILDQKNYVE | |
| | ELNRQLNSTVSSLHSRVDSLEKSNTKLIEELAIAKNNIIKLQEENHQLR | |
| | SENKLILMKTQQHLEEDPKWEFPRKNLVLGKTLGEGEFGKVVKATAFHL | |
| | KGRAGYTTVAVKMLKENASPSELRDLLSEFNVLKQVNHPHVIKLYGACS | |
| | QDGPLLLIVEYAKYGSLRGELRESRKVGPGYLGSGGSRNSSSLDHPDER | |
| | ALTMGDLISFAWQISQGMQYLAEMKLVHRDLAARNILVAEGRKMKISDE | |
| | GLSRDVYEEDSYVKRSQGRIPVKWMAIESLEDHIYTTQSDVWSFGVLLW | |
| | EIVTLGGNPYPGIPPERLENLLKTGHRMERPDNCSEEMYRLMLQCWKQE | |
| | PDKRPVFADISKDLEKMMVKRRDYLDLAASTPSDSLIYDDGLSEEETPL | |
| | VDCNNAPLPRALPSTWIENKLYGRISHAFTRE | |
| SNRNP70- | MTQFLPPNLLALFAPRDPIPYLPPLEKLPHEKHHNQPYCGIAPYIREFE | 121 |
| RET | DPRDAPPPTRAETREERMERKRREKIERRQQEVETELKMWDPHNDPNAQ | |
| | GDAFKTLFVARVNYDTTESKLRREFEVYGPIKREDPKWEFPRKNLVLGK | |
| | TLGEGEFGKVVKATAFHLKGRAGYTTVAVKMLKENASPSELRDLLSEEN | |
| | VLKQVNHPHVIKLYGACSQDGPLLLIVEYAKYGSLRGFLRESRKVGPGY | |
| | LGSGGSRNSSSLDHPDERALTMGDLISFAWQISQGMQYLAEMKLVHRDL | |
| | AARNILVAEGRKMKISDEGLSRDVYEEDSYVKRSQGRIPVKWMAIESLF | |
| | DHIYTTQSDVWSFGVLLWEIVTLGGNPYPGIPPERLENLLKTGHRMERP | |
| | DNCSEEMYRLMLQCWKQEPDKRPVFADISKDLEKMMVKRRDYLDLAAST | |
| | PSDSLIYDDGLSEEETPLVDCNNAPLPRALPSTWIENKLYGRISHAFTR | |
| | F | |
| SQSTM1-RET | MASLTVKAYLLGKEDAAREIRRESFCCSPEPEAEAEAAAGPGPCERLLS | 122 |
| | RVAALFPALRPGGFQAHYRDEDGDLVAFSSDEELTMAMSYVKDDIFRIY | |
| | IKEKKECRRDHRPPCAQEAPRNMVHPNVICDGCNGPVVGTRYKCSVCPD | |
| | YDLCSVCEGKGLHRGHTKLAFPSPFGHLSEGFSHSRWLRKVKHGHEGWP | |
| | GWEMGPPGNWSPRPPRAGEARPGPTAESASGPSEDPSVNFLKNVGESVA | |
| | AALSPLDPLCDELCRTVIAAAVLESFIVSVLLSAFCIHCYHKFAHKPPI | |
| | SSAEMTERRPAQAFPVSYSSSGARRPSLDSMENQVSVDAFKILEDPKWE | |
| | FPRKNLVLGKTLGEGEFGKVVKATAFHLKGRAGYTTVAVKMLKENASPS | |
| | ELRDLLSEFNVLKQVNHPHVIKLYGACSQDGPLLLIVEYAKYGSLRGEL | |
| | RESRKVGPGYLGSGGSRNSSSLDHPDERALTMGDLISFAWQISQGMQYL | |
| | AEMKLVHRDLAARNILVAEGRKMKISDFGLSRDVYEEDSYVKRSQGRIP | |
| | VKWMAIESLFDHIYTTQSDVWSFGVLLWEIVTLGGNPYPGIPPERLENL | |
| | LKTGHRMERPDNCSEEMYRLMLQCWKQEPDKRPVFADISKDLEKMMVKR | |
| | RDYLDLAASTPSDSLIYDDGLSEEETPLVDCNNAPLPRALPSTWIENKL | |
| | YGRISHAFTRF | |
| TNIP1-RET | MEGRGPYRIYDPGGSVPSGEASAAFERLVKENSRLKEKMQGIKMLGELL | 123 |
| | EESQMEATRLRQKAEELVKDNELLPPPSPSLGSFDPLAELTGKDSNVTA | |
| | SPTAPACPSDKPAPVQKPPSSGTSSEFEVVTPEEQNSPESSSHANAMAL | |
| | GPLPREDGNLMLHLQRLETTLSVCAEEPDHGQLFTHLGRMALEFNRLAS | |
| | KVHKNEQRTSILQTLCEQLRKENEALKAKLDKGLEQRDQAAERLREENL | |
| | ELKKLLMSNGNKEGASGRPGSPKMEGTGKKAVAGQQQASVTAGKVPEVV | |
| | ALGAAEKKVKMLEQQRSELLEVNKQWDQHFRSMKQQYEQKITELRQKLA | |
| | DLQKQVTDLEAEREQKQRDFDRKLLLAKSKIEMEETDKEQLTAEAKELR | |
| | QKVKYLQDQLSPLTRQREYQEKEIQRLNKALEEALSIQTPPSSPPTAFG | |
| | SPEGAGALLRKQELVTQNELLKQQEDPKWEFPRKNLVLGKTLGEGEFGK | |
| | VVKATAFHLKGRAGYTTVAVKMLKENASPSELRDLLSEFNVLKQVNHPH | |
| | VIKLYGACSQDGPLLLIVEYAKYGSLRGFLRESRKVGPGYLGSGGSRNS | |
| | SSLDHPDERALTMGDLISFAWQISQGMQYLAEMKLVHRDLAARNILVAE | |
| | GRKMKISDFGLSRDVYEEDSYVKRSQGRIPVKWMAIESLFDHIYTTQSD | |
| | VWSFGVLLWEIVTLGGNPYPGIPPERLENLLKTGHRMERPDNCSEEMYR | |
| | LMLQCWKQEPDKRPVFADISKDLEKMMVKRRDYLDLAASTPSDSLIYDD | |
| | GLSEEETPLVDCNNAPLPRALPSTWIENKLYGRISHAFTRF | |
| TNIP1-RET | MEGRGPYRIYDPGGSVPSGEASAAFERLVKENSRLKEKMQGIKMLGELL | 124 |
| | EESQMEATRLRQKAEELVKDNELLPPPSPSLGSFDPLAELTGKDSNVTA | |
| | SPTAPACPSDKPAPVQKPPSSGTSSEFEVVTPEEQNSPESSSHANAMAL | |
| | GPLPREDGNLMLHLQRLETTLSVCAEEPDHGQLFTHLGRMALEFNRLAS | |
| | KVHKNEQRTSILQTLCEQLRKENEALKAKLDKGLEQRDQAAERLREENL | |
| | ELKKLLMSNGNKEGASGRPGSPKMEGTGKKAVAGQQQASVTAGKVPEVV | |
| | ALGAAEKKVKMLEQQRSELLEVNKQWDQHERSMKQQYEQKITELRQKLA | |
| | DLQKQVTDLEAEREQKQRDFDRKLLLAKSKIEMEETDKEQLTAEAKELR | |
| | QKVKYLQDQLSPLTRQREYQEKEIQRLNKALEEALSIQTPPSSPPTAFG | |
| | SPEGAGALLRKQELVTQNELLKQQVKIFEEDFQRERSDRERMNEEKEEL | |
| | KKQVEKLQAQVTLSNAQLKAFKDEEKAREALRQQKRKAKASGERYHVEP | |
| | HPEHLCGAYPYAYPPMPAMVPHHGFEDWSQIRYPPPPMAMEHPPPLPNS | |
| | RLFHLEDPKWEFPRKNLVLGKTLGEGEFGKVVKATAFHLKGRAGYTTVA | |
| | VKMLKENASPSELRDLLSEFNVLKQVNHPHVIKLYGACSQDGPLLLIVE | |
| | YAKYGSLRGFLRESRKVGPGYLGSGGSRNSSSLDHPDERALTMGDLISF | |
| | AWQISQGMQYLAEMKLVHRDLAARNILVAEGRKMKISDFGLSRDVYEED | |
| | SYVKRSQGRIPVKWMAIESLFDHIYTTQSDVWSFGVLLWEIVTLGGNPY | |
| | PGIPPERLENLLKTGHRMERPDNCSEEMYRLMLQCWKQEPDKRPVFADI | |
| | SKDLEKMMVKRRDYLDLAASTPSDSLIYDDGLSEEETPLVDCNNAPLPR | |
| | ALPSTWIENKLYGRISHAFTRE | |
| TNIP1-RET | MEGRGPYRIYDPGGSVPSGEASAAFERLVKENSRLKEKMQGIKMLGELL | 125 |
| | EESQMEATRLRQKAEELVKDNELLPPPSPSLGSFDPLAELTGKDSNVTA | |
| | SPTAPACPSDKPAPVQKPPSSGTSSEFEVVTPEEQNSPESSSHANAMAL | |
| | GPLPREDGNLMLHLQRLETTLSVCAEEPDHGQLFTHLGRMALEFNRLAS | |
| | KVHKNEQRTSILQTLCEQLRKENEALKAKLDKGLEQRDQAAERLREENL | |
| | ELKKLLMSNGNKEGASGRPGSPKMEGTGKKAVAGQQQASVTAGKVPEVV | |
| | ALGAAEKKVKMLEQQRSELLEVNKQWDQHFRSMKQQYEQKITELRQKLA | |
| | DLQKQVTDLEAEREQKQRDEDRKLLLAKSKIEMEETDKEQLTAEAKELR | |
| | QKVKYLQDQLSPLTRQREYQEKEIQRLNKEDPKWEFPRKNLVLGKTLGE | |
| | GEFGKVVKATAFHLKGRAGYTTVAVKMLKENASPSELRDLLSEENVLKQ | |
| | VNHPHVIKLYGACSQDGPLLLIVEYAKYGSLRGELRESRKVGPGYLGSG | |
| | GSRNSSSLDHPDERALTMGDLISFAWQISQGMQYLAEMKLVHRDLAARN | |
| | ILVAEGRKMKISDFGLSRDVYEEDSYVKRSQGRIPVKWMAIESLEDHIY | |
| | TTQSDVWSFGVLLWEIVTLGGNPYPGIPPERLENLLKTGHRMERPDNCS | |
| | EEMYRLMLQCWKQEPDKRPVFADISKDLEKMMVKRRDYLDLAASTPSDS | |
| | LIYDDGLSEEETPLVDCNNAPLPRALPSTWIENKLYGRISHAFTRF | |
| TRIM27-RET | MASGSVAECLQQETTCPVCLQYFAEPMMLDCGHNICCACLARCWGTAET | 126 |
| | NVSCPQCRETFPQRHMRPNRHLANVTQLVKQLRTERPSGPGGEMGVCEK | |
| | HREPLKLYCEEDQMPICVVCDRSREHRGHSVLPLEEAVEGFKEQIQNQL | |
| | DHLKRVKDLKKRRRAQGEQARAELLSLTQMEREKIVWEFEQLYHSLKEH | |
| | EYRLLARLEELDLAIYNSINGAITQFSCNISHLSSLIAQLEEKQQQPTR | |
| | ELLQEDPKWEFPRKNLVLGKTLGEGEFGKVVKATAFHLKGRAGYTTVAV | |
| | KMLKENASPSELRDLLSEFNVLKQVNHPHVIKLYGACSQDGPLLLIVEY | |
| | AKYGSLRGFLRESRKVGPGYLGSGGSRNSSSLDHPDERALTMGDLISFA | |
| | WQISQGMQYLAEMKLVHRDLAARNILVAEGRKMKISDEGLSRDVYEEDS | |
| | YVKRSQGRIPVKWMAIESLFDHIYTTQSDVWSFGVLLWEIVTLGGNPYP | |
| | GIPPERLENLLKTGHRMERPDNCSEEMYRLMLQCWKQEPDKRPVFADIS | |
| | KDLEKMMVKRRDYLDLAASTPSDSLIYDDGLSEEETPLVDCNNAPLPRA | |
| | LPSTWIENKLYGRISHAFTRF | |
| TRIM33-RET | MAENKGGGEAESGGGGSGSAPVTAGAAGPAAQEAEPPLTAVLVEEEEEE | 127 |
| | GGRAGAEGGAAGPDDGGVAAASSGSAQAASSPAASVGTGVAGGAVSTPA | |
| | PAPASAPAPGPSAGPPPGPPASLLDTCAVCQQSLQSRREAEPKLLPCLH | |
| | SFCLRCLPEPERQLSVPIPGGSNGDIQQVGVIRCPVCRQECRQIDLVDN | |
| | YFVKDTSEAPSSSDEKSEQVCTSCEDNASAVGFCVECGEWLCKTCIEAH | |
| | QRVKFTKDHLIRKKEDVSESVGASGQRPVFCPVHKQEQLKLFCETCDRL | |
| | TCRDCQLLEHKEHRYQFLEEAFQNQKGAIENLLAKLLEKKNYVHFAATQ | |
| | VQNRIKEVNETNKRVEQEIKVAIFTLINEINKKGKSLLQQLENVTKERQ | |
| | MKLLQQQNDITGLSRQVKHVMNFTNWAIASGSSTALLYSKRLITFQLRH | |
| | ILKARCDPVPAANGAIRFHCDPTFWAKNVVNLGNLVIESKPAPGYTPNV | |
| | VVGQVPPGTNHISKTPGQINLAQLRLQHMQQQVYAQKHQQLQQMRMQQP | |
| | PAPVPTTTTTTQQHPRQAAPQMLQQQPPRLISVQTMQRGNMNCGAFQAH | |
| | QMRLAQNAARIPGIPRHSGPQYSMMQPHLQRQEDPKWEFPRKNLVLGKT | |
| | LGEGEFGKVVKATAFHLKGRAGYTTVAVKMLKENASPSELRDLLSEENV | |
| | LKQVNHPHVIKLYGACSQDGPLLLIVEYAKYGSLRGELRESRKVGPGYL | |
| | GSGGSRNSSSLDHPDERALTMGDLISFAWQISQGMQYLAEMKLVHRDLA | |
| | ARNILVAEGRKMKISDEGLSRDVYEEDSYVKRSQGRIPVKWMAIESLED | |
| | HIYTTQSDVWSFGVLLWEIVTLGGNPYPGIPPERLENLLKTGHRMERPD | |
| | NCSEEMYRLMLQCWKQEPDKRPVFADISKDLEKMMVKRRDYLDLAASTP | |
| | SDSLIYDDGLSEEETPLVDCNNAPLPRALPSTWIENKLYGRISHAFTRE | |
| CCDC6-RET | MADSASESDTDGAGGNSSSSAAMQSSCSSTSGGGGGGGGGGGGGKSGGI | 128 |
| | VISPERLEELTNRLASLQQENKVLKIELETYKLKCKALQEENRDLRKAS | |
| | VTIQARAEQEEEFISNTLFKKIQALQKEKETLAVNYEKEEEFLTNELSR | |
| | KLMQLQHEKAELEQHLEQEQEFQVNKLMKKIKKLENDTISKQLTLEQIH | |
| | CATSCAARSQPLSSSPSSSRCCCLPSASTATTSLPTSHPSPQLRPSGGP | |
| | PRPSRSATPLPVPAGPRWTPWRTRSPWMPSRSWRIQSGNSLGRTWFLEK | |
| | LEKANLEKWSRQRPSIKAEQGTPRWPRCKRTPPRVSCETCCQSSTSSRS | |
| | TTHMSSNCMGPAARMARSSSSWSTPNTAPCGASSARAAKWGLATWAVEA | |
| | AATPAPWTTRMSGPSPWATSSHLPGRSHRGCSIWPRSSFIGTWQPETSW | |
| | LRGGRRFRISACPEMEMKRIPTRGARVGFQLNGWQLNPFLIISTPRKVM | |
| | YGLLVSCCGRSPGETPILGFLLSGSSTFRPATGWRGQTTAARRCTACCN | |
| | AGSRSRTKGRCLRTSAKTWRRWLRGETTWTLRRPLHLTPEMTTASQRRR | |
| | HRWWTVIMPPSLEPSLPHGLKTNSMVEFPMHLLDS | |
| TRIM24-RET | MEVAVEKAVAAAAAASAAASGGPSAAPSGENEAESRQGPDSERGGEAAR | 129 |
| | LNLLDTCAVCHQNIQSRAPKLLPCLHSFCQRCLPAPQRYLMLPAPMLGS | |
| | AETPPPVPAPGSPVSGSSPFATQVGVIRCPVCSQECAERHIIDNFFVKD | |
| | TTEVPSSTVEKSNQVCTSCEDNAEANGFCVECVEWLCKTCIRAHQRVKF | |
| | TKDHTVRQKEEVSPEAVGVTSQRPVFCPFHKKEQLKLYCETCDKLTCRD | |
| | CQLLEHKEHRYQFIEEAFQNQKVIIDTLITKLMEKTKYIKFTGNQIQNR | |
| | IIEVNQNQKQVEQDIKVAIFTLMVEINKKGKALLHQLESLAKDHRMKLM | |
| | QQQQEVAGLSKQLEHVMHESKWAVSSGSSTALLYSKRLITYRLRHLLRA | |
| | RCDASPVTNNTIQFHCDPSFWAQNIINLGSLVIEDKESQPQMPKQNPVV | |
| | EQNSQPPSGLSSNQLSKEPTQISLAQLRLQHMQQQQPPPRLINFQNHSP | |
| | KPNGPVLPPHPQQLRYPPNQNIPRQAIKPNPLQMAFLAQQAIKQWQISS | |
| | GQGTPSTTNSTSSTPSSPTITSAAGYDGKAFGSPMIDLSSPVGGSYNLP | |
| | SLPDIDCSSTIMLDNIVRKDTNIDHGQPRPPSNRTVQSPNSSVPSPGLA | |
| | GPVTMTSVHPPIRSPSASSVGSRGSSGSSSKPAGADSTHKVPVVMLEPI | |
| | RIKQENSGPPENYDEPVVIVKQESDEESRPQNANYPRSILTSLLLNSSQ | |
| | SSTSEETVLRSDAPDSTGDQPGLHQDNSSNGKSEWLDPSQKSPLHVGET | |
| | RKEDDPNEDWCAVCQNGGELLCCEKCPKVFHLSCHVPTLTNFPSGEWIC | |
| | TFCRDLSKPEVEYDCDAPSHNSEKKKTEGLVKLTPIDKRKCERLLLFLY | |
| | CHEMSLAFQDPVPLTEDPKWEFPRKNLVLGKTLGEGEFGKVVKATAFHL | |
| | KGRAGYTTVAVKMLKENASPSELRDLLSEENVLKQVNHPHVIKLYGACS | |
| | QDGPLLLIVEYAKYGSLRGFLRESRKVGPGYLGSGGSRNSSSLDHPDER | |
| | ALTMGDLISFAWQISQGMQYLAEMKLVHRDLAARNILVAEGRKMKISDE | |
| | GLSRDVYEEDSYVKRSQGRIPVKWMAIESLFDHIYTTQSDVWSFGVLLW | |
| | EIVTLGGNPYPGIPPERLENLLKTGHRMERPDNCSEEMYRLMLQCWKQE | |
| | PDKRPVFADISKDLEKMMVKRRDYLDLAASTPSDSLIYDDGLSEEETPL | |
| | VDCNNAPLPRALPSTWIENKLYGRISHAFTRE | |
| FGFR1OP- | MAATAAAVVAEEDTELRDLLVQTLENSGVLNRIKAELRAAVFLALEEQE | 130 |
| RET | KVENKTPLVNESLKKFLNTKDGRLVASLVAEFLQFENLDFTLAVFQPET | |
| | STLQGLEGRENLARDLGIIEAEGTVGGPLLLEVIRRCQQKEKGPTTGEG | |
| | ALDLSDVHSPPKSPEGKTSAQTTPSKEDPKWEFPRKNLVLGKTLGEGEF | |
| | GKVVKATAFHLKGRAGYTTVAVKMLKENASPSELRDLLSEFNVLKQVNH | |
| | PHVIKLYGACSQDGPLLLIVEYAKYGSLRGELRESRKVGPGYLGSGGSR | |
| | NSSSLDHPDERALTMGDLISFAWQISQGMQYLAEMKLVHRDLAARNILV | |
| | AEGRKMKISDEGLSRDVYEEDSYVKRSQGRIPVKWMAIESLEDHIYTTQ | |
| | SDVWSFGVLLWEIVTLGGNPYPGIPPERLENLLKTGHRMERPDNCSEEM | |
| | YRLMLQCWKQEPDKRPVFADISKDLEKMMVKRRDYLDLAASTPSDSLIY | |
| | DDGLSEEETPLVDCNNAPLPRALPSTWIENKLYGRISHAFTRE | |
| GAS2-RET | MCTALSPKVRSGPGLSDMHQYSQWLASRHEANLLPMKEDLALWLTNLLG | 131 |
| | KEITAETFMEKLDNGALLCQLAETMQEKFKESMDANKPTKNLPLKKIPC | |
| | KTSAPSGSFFARDNTANFLSWCRDLGVDETCLFESEGLVLHKQPREVCL | |
| | CLLELGRIAARYGVEPPGLIKLEKEIEQEETLSAPSPSPSPSSKSSGKK | |
| | STGNLLDDAVKRISEDPPCKCPNKFCVERLSQGRYRVGEKILFIRVRVC | |
| | PSAAPRTAWRARAGPWTASSGRSTSWWPCAPCTPARARRWWCPSRPCTT | |
| | RTTRRPPSPRASTPPAPWWSSSGRRTPWWPRCVSSMQTWYLHQGSWGGT | |
| | QARCSPGTPGPSRPSGWNTGPTRPRSRPTAASCGRPYMTIGWESTGTSP | |
| | SRRTAPCSWRCWSMTQTSRAQERASSCSTSTCRCCRSACTCPVPTPSPA | |
| | GGLADLPRSGKSVWKTARHSVASTSSTSCIPLVPTAARGWSPQPRTPRG | |
| | SCLMTPRPCGGPSVPNFTTWWWPPTSRPLGRPRPSCLQWRGHMWPRRRA | |
| | APCPVQSARDGWSVRSVAAWAPQQAGVSGGKEMAKGSPGTSPPALPAPR | |
| | PAPTATAMLWRPKTSTFALRTASGAALLGDTSLGSPGGLKLAMAPATAS | |
| | LRRRSASASPKTSRIHCATSCAARSQPLSSSPSSSRCCCLPSASTATTS | |
| | LPTSHPSPQLRPSGGPPRPSRSATPLPVPAGPRWTPWRTRSPWMPSRSW | |
| | RIQSGNSLGRTWFLEKLEKANLEKWSRQRPSIKAEQGTPRWPRCKRTPP | |
| | RVSCETCCQSSTSSRSTTHMSSNCMGPAARMARSSSSWSTPNTAPCGAS | |
| | SARAAKWGLATWAVEAAATPAPWTTRMSGPSPWATSSHLPGRSHRGCSI | |
| | WPRSSFIGTWQPETSWLRGGRRFRISACPEMEMKRIPTRGARVGFQLNG | |
| | WQLNPFLIISTPRKVMYGLLVSCCGRSPGETPILGELLSGSSTERPATG | |
| | WRGQTTAARRCTACCNAGSRSRTKGRCLRTSAKTWRRWLRGETTWTLRR | |
| | PLHLTPFMTTASQRRRHRWWTVIMPPSLEPSLPHGLKTNSMVEFPMHLL | |
| | DS | |
| HOOK1-RET | MEETQPPPQPKLPLCDSLMIWLQTENTASPCQDVKQLTSGVAMAQVLHQ | 132 |
| | IDAAWFNESWLSRIKEDVGDNWRIKASNVKKVLQGIMSYYHEFLGQQIS | |
| | EALIPDLNQITECSDPVELGRLLQLILGCAINCEKKQEHIQNIMTLEES | |
| | VQHVVMTAIQELMSKEILSSPPNDAVGELEQQLKRALEELQEALAEKEE | |
| | LRQRCEELDMQVTTLQDEKNSLVSENEMMNEKLDQLDGSFDDPNTVVAK | |
| | KYFHAQLQLEQLQEENFRLEAAKDDYRVHCEELEKQLIEFQHRNDELTS | |
| | LAEETRALKDEIDVLRATSDKANKLESTVEIYRQKLQDLNDLRKQVKTL | |
| | QETNMMYMHNTVSLEEELKKANAARTQLETYKRQVQDLHVKLSSESKRA | |
| | DTLAFEMKRLEEKHEALLKEKERLIEQRDTLKETNEELRCSQVQQDHLN | |
| | QTDASATKSYENLAAEIMPVEYREVFIRLQHENKMLRLQQEGSENERIE | |
| | ELQEQLEQKHRKMNELETEQRLSKERIRELQQQIEDLQKSLQEQGSKSE | |
| | GESSSKLKQKLEAHMEKLTEVHEELQKKQELIEDLQPDINQNVQKINEL | |
| | EAALQKKDEDMKAMEERYKMYLEKARNVIKTLDPKLNPASAEIMLLRKQ | |
| | LAEKERRIEILEEDPKWEFPRKNLVLGKTLGEGEFGKVVKATAFHLKGR | |
| | AGYTTVAVKMLKENASPSELRDLLSEENVLKQVNHPHVIKLYGACSQDG | |
| | PLLLIVEYAKYGSLRGELRESRKVGPGYLGSGGSRNSSSLDHPDERALT | |
| | MGDLISFAWQISQGMQYLAEMKLVHRDLAARNILVAEGRKMKISDEGLS | |
| | RDVYEEDSYVKRSQGRIPVKWMAIESLFDHIYTTQSDVWSFGVLLWEIV | |
| | TLGGNPYPGIPPERLENLLKTGHRMERPDNCSEEMYRLMLQCWKQEPDK | |
| | RPVFADISKDLEKMMVKRRDYLDLAASTPSDSLIYDDGLSEEETPLVDC | |
| | NNAPLPRALPSTWIENKLYGRISHAFTRE | |
| LMNA-RET | METPSQRRATRSGAQASSTPLSPTRITRLQEKEDLQELNDRLAVYIDRV | 137 |
| | RSLETENAGLRLRITESEEVVSREVSGIKAAYEAELGDARKTLDSVAKE | |
| | RARLQLELSKVREEFKELKARNTKKEGDLIAAQARLKDLEALLNSKEAA | |
| | LSTALSEKRTLEGELHDLRGQVAKEDPKWEFPRKNLVLGKTLGEGEFGK | |
| | VVKATAFHLKGRAGYTTVAVKMLKENASPSELRDLLSEFNVLKQVNHPH | |
| | VIKLYGACSQDGPLLLIVEYAKYGSLRGFLRESRKVGPGYLGSGGSRNS | |
| | SSLDHPDERALTMGDLISFAWQISQGMQYLAEMKLVHRDLAARNILVAE | |
| | GRKMKISDFGLSRDVYEEDSYVKRSQGRIPVKWMAIESLFDHIYTTQSD | |
| | VWSFGVLLWEIVTLGGNPYPGIPPERLENLLKTGHRMERPDNCSEEMYR | |
| | LMLQCWKQEPDKRPVFADISKDLEKMMVKRRDYLDLAASTPSDSLIYDD | |
| | GLSEEETPLVDCNNAPLPRALPSTWIENKLYGRISHAFTRE | |
| LMNA-RET | METPSQRRATRSGAQASSTPLSPTRITRLQEKEDLQELNDRLAVYIDRV | 138 |
| | RSLETENAGLRLRITESEEVVSREVSGIKAAYEAELGDARKTLDSVAKE | |
| | RARLQLELSKVREEFKELKARNTKKEGDLIAAQARLKDLEALLNSKEAA | |
| | LSTALSEKRTLEGELHDLRGQVAKEDPKWEFPRKNLVLGKTLGEGEFGK | |
| | VVKATAFHLKGRAGYTTVAVKMLKENASPSELRDLLSEENVLKQVNHPH | |
| | VIKLYGACSQDGPLLLIVEYAKYGSLRGFLRESRKVGPGYLGSGGSRNS | |
| | SSLDHPDERALTMGDLISFAWQISQGMQYLAEMKLVHRDLAARNILVAE | |
| | GRKMKISDFGLSRDVYEEDSYVKRSQGRIPVKWMAIESLEDHIYTTQSD | |
| | VWSFGVLLWEIVTLGGNPYPGIPPERLENLLKTGHRMERPDNCSEEMYR | |
| | LMLQCWKQEPDKRPVFADISKDLEKMMVKRRDYLDLAASTPSDSLIYDD | |
| | GLSEEETPLVDCNNAPLPRALPSTWIENKLYGRISHAFTRE | |
| MLPH-RET | MGKKLDLSKLTDEEAQHVLEVVQRDEDLRRKEEERLEALKGKIKKESSK | 139 |
| | RELLSDTAHLNETHCARCLQPYQLLVNSKRQCLECGLFTCKSCGRVHPE | |
| | EQGWICDPCHLARVVKIGSLEWYYEHVKARFKREGSAKVIRSLHGRLQG | |
| | GAGPELISEERSGDSDQTDEDGEPGSEAQAQAQPFGSKKKRLLSVHDED | |
| | FEGDSDDSTQPQGHSLHLSSVPEARDSPQSLTDESCSEKAAPHKAEGLE | |
| | EADTGASGCHSHPEEQPTSISPSRHGALAELCPPGGSHRMALGTAAALG | |
| | SNVIRNEQLPLQYLADVDTSDEESIRAHVMASHHSKRRGRASSESQIFE | |
| | LNKHISAVECLLTYLENTVVPPLAKEDPKWEFPRKNLVLGKTLGEGEFG | |
| | KVVKATAFHLKGRAGYTTVAVKMLKENASPSELRDLLSEFNVLKQVNHP | |
| | HVIKLYGACSQDGPLLLIVEYAKYGSLRGFLRESRKVGPGYLGSGGSRN | |
| | SSSLDHPDERALTMGDLISFAWQISQGMQYLAEMKLVHRDLAARNILVA | |
| | EGRKMKISDEGLSRDVYEEDSYVKRSQGRIPVKWMAIESLEDHIYTTQS | |
| | DVWSFGVLLWEIVTLGGNPYPGIPPERLENLLKTGHRMERPDNCSEEMY | |
| | RLMLQCWKQEPDKRPVFADISKDLEKMMVKRRDYLDLAASTPSDSLIYD | |
| | DGLSEEETPLVDCNNAPLPRALPSTWIENKLYGRISHAFTRE | |
| MYH9-RET | MAQQAADKYLYVDKNFINNPLAQADWAAKKLVWVPSDKSGFEPASLKEE | 140 |
| | VGEEAIVELVENGKKVKVNKDDIQKMNPPKESKVEDMAELTCLNEASVL | |
| | HNLKERYYSGLIYTYSGLFCVVINPYKNLPIYSEEIVEMYKGKKRHEMP | |
| | PHIYAITDTAYRSMMQDREDQSILCTGESGAGKTENTKKVIQYLAYVAS | |
| | SHKSKKDQGELERQLLQANPILEAFGNAKTVKNDNSSRFGKFIRINFDV | |
| | NGYIVGANIETYLLEKSRAIRQAKEERTFHIFYYLLSGAGEHLKTDLLL | |
| | EPYNKYRFLSNGHVTIPGQQDKDMFQETMEAMRIMGIPEEEQMGLLRVI | |
| | SGVLQLGNIVEKKERNTDQASMPDNTAAQKVSHLLGINVTDFTRGILTP | |
| | RIKVGRDYVQKAQTKEQADFAIEALAKATYERMERWLVLRINKALDKTK | |
| | RQGASFIGILDIAGFEIFDLNSFEQLCINYTNEKLQQLENHTMFILEQE | |
| | EYQREGIEWNFIDFGLDLQPCIDLIEKPAGPPGILALLDEECWFPKATD | |
| | KSFVEKVMQEQGTHPKFQKPKQLKDKADFCIIHYAGKVDYKADEWLMKN | |
| | MDPLNDNIATLLHQSSDKFVSELWKDVDRIIGLDQVAGMSETALPGAFK | |
| | TRKGMFRTVGQLYKEQLAKLMATLRNTNPNFVRCIIPNHEKKAGKLDPH | |
| | LVLDQLRCNGVLEGIRICRQGFPNRVVFQEFRQRYEILTPNSIPKGEMD | |
| | GKQACVLMIKALELDSNLYRIGQSKVFFRAGVLAHLEEERDLKITDVII | |
| | GFQACCRGYLARKAFAKRQQQLTAMKVLQRNCAAYLKLRNWQWWRLFTK | |
| | VKPLLQVSRQEEEMMAKEEELVKVREKQLAAENRLTEMETLQSQLMAEK | |
| | LQLQEQLQAETELCAEAEELRARLTAKKQELEEICHDLEARVEEEEERC | |
| | QHLQAEKKKMQQNIQELEEQLEEEESARQKLQLEKVTTEAKLKKLEEEQ | |
| | IILEDQNCKLAKEKKLLEDRIAEFTTNLTEEEEKSKSLAKLKNKHEAMI | |
| | TDLEERLRREEKQRQELEKTRRKLEGDSTDLSDQIAELQAQIAELKMQL | |
| | AKKEEELQAALARVEEEAAQKNMALKKIRELESQISELQEDLESERASR | |
| | NKAEKQKRDLGEELEALKTELEDTLDSTAAQQELRSKREQEVNILKKTL | |
| | EEEAKTHEAQIQEMRQKHSQAVEELAEQLEQTKRVKANLEKAKQTLENE | |
| | RGELANEVKVLLQGKGDSEHKRKKVEAQLQELQVKFNEGERVRTELADK | |
| | VTKLQVELDNVTGLLSQSDSKSSKLTKDFSALESQLQDTQELLQEENRQ | |
| | KLSLSTKLKQVEDEKNSFREQLEEEEEAKHNLEKQIATLHAQVADMKKK | |
| | MEDSVGCLETAEEVKRKLQKDLEGLSQRHEEKVAAYDKLEKTKTRLQQE | |
| | LDDLLVDLDHQRQSACNLEKKQKKFDQLLAEEKTISAKYAEERDRAEAE | |
| | AREKETKALSLARALEEAMEQKAELERLNKQFRTEMEDLMSSKDDVGKS | |
| | VHELEKSKRALEQQVEEMKTQLEELEDELQATEDAKLRLEVNLQAMKAQ | |
| | FERDLQGRDEQSEEKKKQLVRQVREMEAELEDERKQRSMAVAARKKLEM | |
| | DLKDLEAHIDSANKNRDEAIKQLRKLQEDPKWEFPRKNLVLGKTLGEGE | |
| | FGKVVKATAFHLKGRAGYTTVAVKMLKENASPSELRDLLSEENVLKQVN | |
| | HPHVIKLYGACSQDGPLLLIVEYAKYGSLRGELRESRKVGPGYLGSGGS | |
| | RNSSSLDHPDERALTMGDLISFAWQISQGMQYLAEMKLVHRDLAARNIL | |
| | VAEGRKMKISDFGLSRDVYEEDSYVKRSQGRIPVKWMAIESLEDHIYTT | |
| | QSDVWSFGVLLWEIVTLGGNPYPGIPPERLENLLKTGHRMERPDNCSEE | |
| | MYRLMLQCWKQEPDKRPVFADISKDLEKMMVKRRDYLDLAASTPSDSLI | |
| | YDDGLSEEETPLVDCNNAPLPRALPSTWIENKLYGRISHAFTRF | |
| PCDH15-RET | MFRQFYLWTCLASGIILGSLFEICLGQYDDGHRGGHAACLRCRRGTCIR | 141 |
| | GAGEAVHKHAAPRGHLGPADLPGGTLAQRDLGPGQRQLRAGDRTLAGSQ | |
| | PEPLHLGEPHHAAGGAGQLRLPGPRSGRPLAPLQRVGAAGQPAPAQYLL | |
| | PLREQEGSPICPDRESLCGKLPGIQWHQRPVQAAFLWCQLQHARGGHLS | |
| | RGHLGDPVCEHQGPAAAQVCRTSLHGGGHRPADLAGPGPAACNSGGVIC | |
| | GRGGGLPPVLCSQQETAGVGVWRPGLPNRQVVEARRWQRDHQELLHLLS | |
| | QHQDLPRRPLRCCGDPRHQHLPSGLPPGQHCWGTRAWGAPGDSWLWHLQ | |
| | LLPGGEVLLRARRHPGSTVRRAVPHGDRSRCPLLLHRLGAAVCLLHPLL | |
| | PQVCPQATHLLSDDLPEARPGLPGQLLLERCPPALAGLHGEPGLRGCLQ | |
| | DPGGSKVGIPSEELGSWKNSRRRRIWKSGQGNGLPSERQSRVHHGGRED | |
| | AERERLPEAARPAVRVQRPEAGQPPTCHQIVWGLQPGWPAPPHRGVRQI | |
| | RLPAGLPPREPQSGAWLPGQWRQPQLQLPGPPGAGPHHGRPHLICLADL | |
| | TGDAVSGRDEARSSGLGSQKHPGSGAEDEDEGFRLVPRCLRGELREEEP | |
| | GSDSSMDGNIPFSYLHHAKCMVFWCPAVGDRDPRGKPLSWDSSAALQPS | |
| | EDRPPDGEARQLQRGDVPPDAAMLEAGAGQKAGVCGHQQRPGEDDGEER | |
| | LLGPCGVHSILPDLRRPLRGGDTAGGLCPPPSSPPFHMDKQTLWNFPCI | |
| | YIL | |
| PIBF1-RET | MSRKISKESKKVNISSSLESEDISLETTVPTDDISSSEEREGKVRITRQ | 142 |
| | LIERKELLHNIQLLKIELSQKTMMIDNLKVDYLTKIEELEEKLNDALHQ | |
| | KQLLTLRLDNQLAFQQKDASKYQELMKQEMETILLRQKQLEETNLQLRE | |
| | KAGDVRRNLRDFELTEEQYIKLKAFPEDQLSIPEYVSVRFYELVNPLRK | |
| | EICELQVKKNILAEELSTNKNQLKQLTETYEEDRKNYSEVQIRCQRLAL | |
| | ELADTKQLIQQGDYRQENYDKVKSERDALEQEVIELRRKHEILEASHMI | |
| | QTKERSELSKEVVTLEQTVTLLQKDKEYLNRQNMELSVRCAHEEDRLER | |
| | LQAQLEESKKAREEMYEKYVASRDHYKTEYENKLHDELEQIRLKTNQEI | |
| | DQLRNASREMYERENRNLREARDNAVAEKERAVMAEKDALEKHDQLLDR | |
| | YRELQLSTESKVTEFLHQSKLKSFESERVQLLQEETARNLTQCQLECEK | |
| | YQKKLEVLTKEFYSLQASSEKRITELQAQNSEHQARLDIYEKLEKELDE | |
| | IIMQTAEIENEDEAERVLESYGYGANVPTTAKRRLKQSVHLARRVLQLE | |
| | KQNSLILKDLEHRKDQVTQLSQELDRANSLLNQTQQPYRYLIESVRQRD | |
| | SKIDSLTESIAQLEKDVSNLNKEKSALLQTKNQMALDLEQLLNHREEDP | |
| | KWEFPRKNLVLGKTLGEGEFGKVVKATAFHLKGRAGYTTVAVKMLKENA | |
| | SPSELRDLLSEFNVLKQVNHPHVIKLYGACSQDGPLLLIVEYAKYGSLR | |
| | GELRESRKVGPGYLGSGGSRNSSSLDHPDERALTMGDLISFAWQISQGM | |
| | QYLAEMKLVHRDLAARNILVAEGRKMKISDFGLSRDVYEEDSYVKRSQG | |
| | RIPVKWMAIESLEDHIYTTQSDVWSFGVLLWEIVTLGGNPYPGIPPERL | |
| | FNLLKTGHRMERPDNCSEEMYRLMLQCWKQEPDKRPVFADISKDLEKMM | |
| | VKRRDYLDLAASTPSDSLIYDDGLSEEETPLVDCNNAPLPRALPSTWIE | |
| | NKLYGRISHAFTRF | |
| PIBF1-RET | MSRKISKESKKVNISSSLESEDISLETTVPTDDISSSEEREGKVRITRQ | 143 |
| | LIERKELLHNIQLLKIELSQKTMMIDNLKVDYLTKIEELEEKLNDALHQ | |
| | KQLLTLRLDNQLAFQQKDASKYQELMKQEMETILLRQKQLEETNLQLRE | |
| | KAGDVRRNLRDFELTEEQYIKLKAFPEDQLSIPEYVSVRFYELVNPLRK | |
| | EICELQVKKNILAEELSTNKNQLKQLTETYEEDRKNYSEVQIRCQRLAL | |
| | ELADTKQLIQQGDYRQENYDKVKSERDALEQEVIELRRKHEILEASHMI | |
| | QTKERSELSKEVVTLEQTVTLLQKDKEYLNRQNMELSVRCAHEEDRLER | |
| | LQAQLEESKKAREEMYEKYVASRDHYKTEYENKLHDELEQIRLKTNQEI | |
| | DQLRNASREMYERENRNLREARDNAVAEKERAVMAEKDALEKHDQLLDR | |
| | YRELQLSTESKVTEFLHQSKLKSFESERVQLLQEETARNLTQCQLECEK | |
| | YQKKLEVLTKEFYSLQASSEKRITELQAQNSEHQARLDIYEKLEKELDE | |
| | IIMQTAEIENEDEAERVLFSYGYGANVPTTAKRRLKQSVHLARRVLQLE | |
| | KQNSLILKDLEHRKDQVTQLSQELDRANSLLNQTQQPYRYLIESVRQRD | |
| | SKIDSLTESIAQLEKDVSNLNKEKSALLQTKNQMALDLEQLLNHREEDP | |
| | KWEFPRKNLVLGKTLGEGEFGKVVKATAFHLKGRAGYTTVAVKMLKENA | |
| | SPSELRDLLSEFNVLKQVNHPHVIKLYGACSQDGPLLLIVEYAKYGSLR | |
| | GELRESRKVGPGYLGSGGSRNSSSLDHPDERALTMGDLISFAWQISQGM | |
| | QYLAEMKLVHRDLAARNILVAEGRKMKISDEGLSRDVYEEDSYVKRSQG | |
| | RIPVKWMAIESLEDHIYTTQSDVWSFGVLLWEIVTLGGNPYPGIPPERL | |
| | FNLLKTGHRMERPDNCSEEMYRLMLQCWKQEPDKRPVFADISKDLEKMM | |
| | VKRRDYLDLAASTPSDSLIYDDGLSEEETPLVDCNNAPLPRALPSTWIE | |
| | NKLYGRISHAFTRE | |
| SLC12A2- | MEPRPTAPSSGAPGLAGVGETPSAAALAAARVELPGTAVPSVPEDAAPA | 144 |
| RET | SRDGGGVRDEGPAAAGDGLGRPLGPTPSQSRFQVDLVSENAGRAAAAAA | |
| | AAAAAAAAAGAGAGAKQTPADGEASGESEPAKGSEEAKGRERVNFVDPA | |
| | ASSSAEDSLSDAAGVGVDGPNVSFQNGGDTVLSEGSSLHSGGGGGSGHH | |
| | QHYYYDTHTNTYYLRTFGHNTMDAVPRIDHYRHTAAQLGEKLLRPSLAE | |
| | LHDELEKEPFEDGFANGEESTPTRDAVVTYTAESKGVVKFGWIKGVLVR | |
| | CMLNIWGVMLFIRLSWIVGQAGIGLSVLVIMMATVVTTITGLSTSAIAT | |
| | NGFVRGGGAYYLISRSLGPEFGGAIGLIFAFANAVAVAMYVVGFAETVV | |
| | ELLKEHSILMIDEINDIRIIGAITVVILLGISVAGMEWEAKAQIVLLVI | |
| | LLLAIGDFVIGTFIPLESKKPKGFFGYKSEIFNENFGPDFREEETFFSV | |
| | FAIFFPAATGILAGANISGDLADPQSAIPKGTLLAILITTLVYVGIAVS | |
| | VGSCVVRDATGNVNDTIVTELTNCTSAACKLNFDESSCESSPCSYGLMN | |
| | NFQVMSMVSGFTPLISAGIFSATLSSALASLVSAPKIFQALCKDNIYPA | |
| | FQMFAKGYGKNNEPLRGYILTFLIALGFILIAELNVIAPIISNFFLASY | |
| | ALINFSVFHASLAKSPGWRPAFKYYNMWISLLGAILCCIVMEVINWWAA | |
| | LLTYVIVLGLYIYVTYKKPDVNWGSSTQALTYLNALQHSIRLSGVEDHV | |
| | KNFRPQCLVMTGAPNSRPALLHLVHDFTKNVGLMICGHVHMEDPKWEFP | |
| | RKNLVLGKTLGEGEFGKVVKATAFHLKGRAGYTTVAVKMLKENASPSEL | |
| | RDLLSEFNVLKQVNHPHVIKLYGACSQDGPLLLIVEYAKYGSLRGELRE | |
| | SRKVGPGYLGSGGSRNSSSLDHPDERALTMGDLISFAWQISQGMQYLAE | |
| | MKLVHRDLAARNILVAEGRKMKISDEGLSRDVYEEDSYVKRSQGRIPVK | |
| | WMAIESLFDHIYTTQSDVWSFGVLLWEIVTLGGNPYPGIPPERLENLLK | |
| | TGHRMERPDNCSEEMYRLMLQCWKQEPDKRPVFADISKDLEKMMVKRRD | |
| | YLDLAASTPSDSLIYDDGLSEEETPLVDCNNAPLPRALPSTWIENKLYG | |
| | RISHAFTRE | |
| ZNF485-RET | MAPRAQIQGPLTFGDVAVAFTRIEWRHLDAAQRALYRDVMLENYGNLVS | 145 |
| | VGLLSSKPKLITQLEQGAEPWTEVREAPSGTHAGGSKVGIPSEELGSWK | |
| | NSRRRRIWKSGQGNGLPSERQSRVHHGGREDAERERLPEAARPAVRVQR | |
| | PEAGQPPTCHQIVWGLQPGWPAPPHRGVRQIRLPAGLPPREPQSGAWLP | |
| | GQWRQPQLQLPGPPGAGPHHGRPHLICLADLTGDAVSGRDEARSSGLGS | |
| | QKHPGSGAEDEDFGFRLVPRCLRGFLREEEPGSDSSMDGNIPFSYLHHA | |
| | KCMVFWCPAVGDRDPRGKPLSWDSSAALQPSEDRPPDGEARQLQRGDVP | |
| | PDAAMLEAGAGQKAGVCGHQQRPGEDDGEERLLGPCGVHSILPDLRRPL | |
| | RGGDTAGGLCPPPSSPPFHMDKQTLWNEPCIYIL | |
| ZNF485-RET | MAPRAQIQGPLTFGDVAVAFTRIEWRHLDAAQRALYRDVMLENYGNLVS | 146 |
| | VGLLSSKPKLITQLEQGAEPWTEVREAPSGTHAGGSIVGGHEPGEPRGI | |
| | KAGYGTCNCFPEEEKCFCEPEDIQDPLCDELCRTVIAAAVLESFIVSVL | |
| | LSAFCIHCYHKFAHKPPISSAEMTFRRPAQAFPVSYSSSGARRPSLDSM | |
| | ENQVSVDAFKILEDPKWEFPRKNLVLGKTLGEGEFGKVVKATAFHLKGR | |
| | AGYTTVAVKMLKENASPSELRDLLSEENVLKQVNHPHVIKLYGACSQDG | |
| | PLLLIVEYAKYGSLRGFLRESRKVGPGYLGSGGSRNSSSLDHPDERALT | |
| | MGDLISFAWQISQGMQYLAEMKLVHRDLAARNILVAEGRKMKISDFGLS | |
| | RDVYEEDSYVKRSQGRIPVKWMAIESLEDHIYTTQSDVWSFGVLLWEIV | |
| | TLGGNPYPGIPPERLENLLKTGHRMERPDNCSEEMYRLMLQCWKQEPDK | |
| | RPVFADISKDLEKMMVKRRDYLDLAASTPSDSLIYDDGLSEEETPLVDC | |
| | NNAPLPRALPSTWIENKLYGRISHAFTRF | |

In some embodiments, the RET fusion polypeptide of the disclosure is a GOLGB1-RET fusion polypeptide comprising the amino acid sequence of SEQ ID NO: 68 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a GP2-RET fusion polypeptide comprising the amino acid sequence of SEQ ID NO: 69 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a PPP2R5A-RET fusion polypeptide comprising the amino acid sequence of SEQ ID NO: 70 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a GRM7-RET fusion polypeptide comprising the amino acid sequence of SEQ ID NO: 72 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a MYH14-RET fusion polypeptide comprising the amino acid sequence of SEQ ID NO: 74 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a MGEA5-RET fusion polypeptide comprising the amino acid sequence of SEQ ID NO: 75 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a SAMD4A-RET fusion polypeptide comprising the amino acid sequence of SEQ ID NO: 76 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a ACPP-RET fusion polypeptide comprising the amino acid sequence of SEQ ID NO: 77 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a BMS1-RET fusion polypeptide comprising the amino acid sequence of SEQ ID NO: 78 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a CEP135-RET fusion polypeptide comprising the amino acid sequence of SEQ ID NO: 79 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a EEA1-RET fusion polypeptide comprising the amino acid sequence of SEQ ID NO: 80 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a CSGALNACT2-RET fusion polypeptide comprising the amino acid sequence of SEQ ID NO: 81 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a KIAA1217-RET fusion polypeptide comprising the amino acid sequence of SEQ ID NO: 82 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a SORBS1-RET fusion polypeptide comprising the amino acid sequence of SEQ ID NO: 83 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a SORBS1-RET fusion polypeptide comprising the amino acid sequence of SEQ ID NO: 84 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a MPRIP-RET fusion polypeptide comprising the amino acid sequence of SEQ ID NO: 85 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a TFG-RET fusion polypeptide comprising the amino acid sequence of SEQ ID NO: 86 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a SPECC1L-RET fusion polypeptide comprising the amino acid sequence of SEQ ID NO: 87 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a REEP3-RET fusion polypeptide comprising the amino acid sequence of SEQ ID NO: 88 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a RRBP1-RET fusion polypeptide comprising the amino acid sequence of SEQ ID NO: 89 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a ETV6-RET fusion polypeptide comprising the amino acid sequence of SEQ ID NO: 90 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a TAF3-RET fusion polypeptide comprising the amino acid sequence of SEQ ID NO: 91 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a PCM1-RET fusion polypeptide comprising the amino acid sequence of SEQ ID NO: 92 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a PCM1-RET fusion polypeptide comprising the amino acid sequence of SEQ ID NO: 93 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a TNIP2-RET fusion polypeptide comprising the amino acid sequence of SEQ ID NO: 94 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a SATB1-RET fusion polypeptide comprising the amino acid sequence of SEQ ID NO: 95 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a RET-ADCY1 fusion polypeptide comprising the amino acid sequence of SEQ ID NO: 96 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a RET-ZNF248 fusion polypeptide comprising the amino acid sequence of SEQ ID NO: 97 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a RET-AGBL4 fusion polypeptide comprising the amino acid sequence of SEQ ID NO: 98 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a RET-LRMDA fusion polypeptide comprising the amino acid sequence of SEQ ID NO: 99 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a RET-ARHGAP19 fusion polypeptide comprising the amino acid sequence of SEQ ID NO: 100 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a RET-CPEB3 fusion polypeptide comprising the amino acid sequence of SEQ ID NO: 101 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a RET-DCC fusion polypeptide comprising the amino acid sequence of SEQ ID NO: 102 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a RET-ELMO1 fusion polypeptide comprising the amino acid sequence of SEQ ID NO: 103 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a RET-WDFY4 fusion polypeptide comprising the amino acid sequence of SEQ ID NO: 104 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is an ANKRD26-RET fusion polypeptide comprising the amino acid sequence of SEQ ID NO: 105 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a CLIP1-RET fusion polypeptide comprising the amino acid sequence of SEQ ID NO: 106 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a DLG5-RET fusion polypeptide comprising the amino acid sequence of SEQ ID NO: 107 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a DLG5-RET fusion polypeptide comprising the amino acid sequence of SEQ ID NO: 108 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is an ERC1-RET fusion polypeptide comprising the amino acid sequence of SEQ ID NO: 109 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is an ERC1-RET fusion polypeptide comprising the amino acid sequence of SEQ ID NO: 110 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is an ERC1-RET fusion polypeptide comprising the amino acid sequence of SEQ ID NO: 111 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a FRMD4A-RET fusion polypeptide comprising the amino acid sequence of SEQ ID NO: 112 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a KIAA1468-RET fusion polypeptide comprising the amino acid sequence of SEQ ID NO: 113 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a NCOA4-RET fusion polypeptide comprising the amino acid sequence of SEQ ID NO: 114 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a PARD3-RET fusion polypeptide comprising the amino acid sequence of SEQ ID NO: 115 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a PARD3-RET fusion polypeptide comprising the amino acid sequence of SEQ ID NO: 116 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a PRKAR1A-RET fusion polypeptide comprising the amino acid sequence of SEQ ID NO: 117 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a PRKG1-RET fusion polypeptide comprising the amino acid sequence of SEQ ID NO: 118 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a PRKG1-RET fusion polypeptide comprising the amino acid sequence of SEQ ID NO: 119 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a RUFY2-RET fusion polypeptide comprising the amino acid sequence of SEQ ID NO: 120 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a SNRNP70-RET fusion polypeptide comprising the amino acid sequence of SEQ ID NO: 121 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a SQSTM1-RET fusion polypeptide comprising the amino acid sequence of SEQ ID NO: 122 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a TNIP1-RET fusion polypeptide comprising the amino acid sequence of SEQ ID NO: 123 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a TNIP1-RET fusion polypeptide comprising the amino acid sequence of SEQ ID NO: 124 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a TNIP1-RET fusion polypeptide comprising the amino acid sequence of SEQ ID NO: 125 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a TRIM27-RET fusion polypeptide comprising the amino acid sequence of SEQ ID NO: 126 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a TRIM33-RET fusion polypeptide comprising the amino acid sequence of SEQ ID NO: 127 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a CCDC6-RET fusion polypeptide comprising the amino acid sequence of SEQ ID NO: 128 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a TRIM24-RET fusion polypeptide comprising the amino acid sequence of SEQ ID NO: 129 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a FGFR1OP-RET fusion polypeptide comprising the amino acid sequence of SEQ ID NO: 130 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a GAS2-RET fusion polypeptide comprising the amino acid sequence of SEQ ID NO: 131 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a HOOK1-RET fusion polypeptide comprising the amino acid sequence of SEQ ID NO: 132 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a LMNA-RET fusion polypeptide comprising the amino acid sequence of SEQ ID NO: 137 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a LMNA-RET fusion polypeptide comprising the amino acid sequence of SEQ ID NO: 138 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a MLPH-RET fusion polypeptide comprising the amino acid sequence of SEQ ID NO: 139 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a MYH9-RET fusion polypeptide comprising the amino acid sequence of SEQ ID NO: 140 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a PCDH15-RET fusion polypeptide comprising the amino acid sequence of SEQ ID NO: 141 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a PIBF1-RET fusion polypeptide comprising the amino acid sequence of SEQ ID NO: 142 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a PIBF1-RET fusion polypeptide comprising the amino acid sequence of SEQ ID NO: 143 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a SLC12A2-RET fusion polypeptide comprising the amino acid sequence of SEQ ID NO: 144 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a ZNF485-RET fusion polypeptide comprising the amino acid sequence of SEQ ID NO: 145 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the RET fusion polypeptide of the disclosure is a ZNF485-RET fusion polypeptide comprising the amino acid sequence of SEQ ID NO: 146 or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto.

In some embodiments, the RET fusion polypeptide of the disclosure comprises an amino acid sequence encoded by any of the RET fusion nucleic acid molecules as described in the Examples, herein.

In some embodiments of any of the RET fusion polypeptides provided herein, the fusion polypeptide is an isolated polypeptide.

(iii) Cancers and Methods Related Thereto

Certain aspects of the present disclosure relate to methods for identifying an individual having a cancer who may benefit from a treatment comprising an anti-cancer therapy; selecting a treatment or therapy for an individual having a cancer; identifying one or more treatment options for an individual having a cancer; predicting survival of an individual having a cancer; treating or delaying progression of cancer; monitoring, evaluating or screening an individual having a cancer; assessing a RET fusion nucleic acid molecule or a RET fusion polypeptide in a cancer in an individual; detecting the presence or absence of a cancer in an individual; detecting the presence or absence of a RET fusion nucleic acid molecule or a RET fusion polypeptide in a cancer in an individual; monitoring progression or recurrence of a cancer in an individual; or identifying a candidate treatment for a cancer in an individual in need thereof.

In some embodiments of any of the methods provided herein, the methods comprise acquiring knowledge of or detecting in a sample from an individual having cancer, suspected of having cancer, being tested for cancer, or being treated for cancer, a RET fusion nucleic acid molecule of the disclosure (e.g., any of the RET fusion nucleic acid molecules described above, in Tables 1-8, and/or in the Examples herein). In other embodiments, the methods comprise acquiring knowledge of or detecting in a sample from an individual having cancer, suspected of having cancer, being tested for cancer, or being treated for cancer, a RET fusion polypeptide of the disclosure (e.g., a RET fusion polypeptide encoded by any of the RET fusion nucleic acid molecules of the disclosure, or a RET fusion polypeptide as described above, in Table 9, and/or in the Examples herein).

In some embodiments of any of the methods provided herein, detection of a RET fusion nucleic acid molecule or a RET fusion polypeptide of the disclosure in a sample from an individual (e.g., an individual having cancer, suspected of having cancer, being tested for cancer, or being treated for cancer) identifies the individual as one who may benefit from a treatment comprising an anti-cancer therapy, such as an anti-cancer therapy provided herein, e.g., a RET-targeted therapy.

In some embodiments, the methods comprise detecting, in a first sample obtained from the individual at a first time point, the presence or absence of a RET fusion nucleic acid molecule or a RET fusion polypeptide of the disclosure. In some embodiments, the methods further comprise detecting, in a second sample obtained from the individual at a second time point after the first time point, the presence or absence of a RET fusion nucleic acid molecule or a RET fusion polypeptide of the disclosure. In some embodiments, the methods further comprise providing an assessment of cancer progression or cancer recurrence in the individual based, at least in part, on the presence or absence of the RET fusion nucleic acid molecule or the RET fusion polypeptide in the first sample and/or in the second sample. In some embodiments, the presence of the RET fusion nucleic acid molecule or the RET fusion polypeptide in the first sample and/or in the second sample identifies the individual as having increased risk of cancer progression or cancer recurrence. In some embodiments, the methods further comprise selecting a treatment, administering a treatment, adjusting a treatment, adjusting a dose of a treatment, or applying a treatment to the individual based, at least in part, on detecting the presence of the RET fusion nucleic acid molecule or polypeptide in the first sample and/or in the second sample, wherein the treatment comprises an anti-cancer therapy, such as an anti-cancer therapy provided herein, e.g., a RET-targeted therapy.

In some embodiments, the methods comprise performing DNA sequencing on a sample obtained from an individual (e.g., an individual having cancer, suspected of having cancer, being tested for cancer, or being treated for cancer) to determine a sequencing mutation profile on a group of genes. In some embodiments, the group of genes comprises one or more known/suspected oncogenes and/or tumor suppressors, one or more cancer-related genes, or any combination thereof. Alternatively or additionally, in some embodiments, the group of genes comprises one or more of ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNFIA, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MSTIR, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2RIA, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSCIL1, WT1, XPO1, XRCC2, ZNF217, ZNF703, or any combination thereof. Alternatively or additionally, in some embodiments, the group of genes comprises one or more of ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1B, IL-6, IL-6R, JAK1, JAK2, JAK3, KIT, KRAS, MEK, MET, MSI-H, mTOR, PARP, PD-1, PDGFR, PDGFRa, PDGFRβ, PD-L1, PI3K8, PIGF, PTCH, RAF, RANKL, RET, ROS1, SLAMF7, VEGF, VEGFA, VEGFB, or any combination thereof. Alternatively or additionally, in some embodiments, the group of genes comprises one or more of RET, CPEB3, OPTN, GRM7, AGBL4, PPP2R5A, OXR1, LRMDA, MGEA5, ADCY1, SAMD4A, HSD17B7P2, ACPP, ZNF248, MKX, MYH14, BMS1, GOLGB1, NTRK2, ABI3BP, ARHGAP19, GP2, SH2D3A, VSTM4, RBMS3, LINC00379, ZNF721, ZSWIM6, SGIP1, DCC, CCNY, WDFY4, EEA1, ELMO1, RAD1, NAALADL2, CCBE1, CEP135, RAI14, ADAMTS14, SORBS1, CSGALNACT2, PCM1, KIAA1217, MPRIP, TFG, SPECC1L, REEP3, RRBP1, ETV6, TAF3, RASGEF1A, TNIP2, SATB1, ALOX5, ANKRD26, CLIP1, DLG5, ERC1, FRMD4A, KIAA1468, NCOA4, PARD3, PRKAR1A, PRKG1, RUFY2, SNRNP70, SQSTM1, TNIP1, TRIM27, TRIM33, CCDC6, TRIM24, FGFR1OP, GAS2, HOOK1, LMNA, MLPH, MYH9, PCDH15, PIBF1, SLC12A2, or ZNF485, or any gene listed in Tables 1-2, and any combination thereof. In some embodiments, the sequencing mutation profile identifies the presence or absence of a RET fusion nucleic acid molecule of the disclosure. In some embodiments, the methods further comprise identifying a candidate treatment for a cancer in an individual, based at least in part on the sequencing mutation profile. In some embodiments, the candidate treatment comprises an anti-cancer therapy, such as an anti-cancer therapy provided herein, e.g., a RET-targeted therapy. In some embodiments, the sequencing mutation profile identifies the presence or absence of a fragment of the RET fusion nucleic acid molecule, optionally wherein the fragment comprises a breakpoint or fusion junction, e.g., one or more of the corresponding breakpoints described herein. In some embodiments, the fragment comprises any of at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more, nucleotides in length. In some embodiments, the fragment comprises between about 5 and about 100 nucleotides, between about 10 and about 50 nucleotides, or between about 10 and about 20 nucleotides, including any specific value within each of the recited ranges. In some embodiments, the fragment comprises any of at least about 1, 2, 3, 4, 5, 6, 7, 8, 9, or 10 nucleotides on either side of the breakpoint or fusion junction, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more, nucleotides on either side of the breakpoint or fusion junction. In some embodiments, the presence of the RET fusion nucleic acid molecule in the sample identifies the individual as one who may benefit from a treatment comprising an anti-cancer therapy, e.g., an anti-cancer therapy provided herein, such as a RET-targeted therapy. In some embodiments, the presence of the RET fusion nucleic acid molecule in the sample predicts the individual to have longer survival when treated with a treatment comprising an anti-cancer therapy, e.g., a RET-targeted therapy, as compared to survival of an individual whose cancer does not comprise a RET fusion nucleic acid molecule.

In some embodiments of any of the methods provided herein, the methods further comprise generating a report comprising one or more treatment options identified for the individual based at least in part on detection of the RET fusion nucleic acid molecule or the RET fusion polypeptide in the sample, wherein the one or more treatment options comprise an anti-cancer therapy, such as an anti-cancer therapy provided herein, e.g., a RET-targeted therapy.

In some embodiments of any of the methods provided herein, responsive to acquisition of knowledge of a RET fusion nucleic acid molecule or a RET fusion polypeptide of the disclosure in a sample from an individual (e.g., an individual having cancer, suspected of having cancer, being tested for cancer, or being treated for cancer): (i) the individual is classified as a candidate to receive a treatment comprising an anti-cancer therapy, such as an anti-cancer therapy provided herein, e.g., a RET-targeted therapy; and/or (ii) the individual is identified as likely to respond to a treatment that comprises an anti-cancer therapy, such as an anti-cancer therapy provided herein, e.g., a RET-targeted therapy. In some embodiments, responsive to acquisition of knowledge of the RET fusion nucleic acid molecule or a RET fusion polypeptide in a sample from the individual, the individual is predicted to have longer survival when treated with a treatment comprising an anti-cancer therapy, such as an anti-cancer therapy provided herein, e.g., a RET-targeted therapy, as compared to survival of an individual whose cancer does not comprise or exhibit a RET fusion nucleic acid molecule or polypeptide. In some embodiments, responsive to acquisition of knowledge of the RET fusion nucleic acid molecule or the RET fusion polypeptide in a sample from the individual, the individual is predicted to have resistance to an anti-cancer therapy (e.g., a non-RET-targeted therapy, and/or a prior anti-cancer therapy administered to the individual), the individual is predicted to respond to an anti-cancer therapy (e.g., an anti-cancer therapy provided herein, such as a RET-targeted therapy), and/or the individual is predicted to have poor prognosis, e.g., when treated with a non-RET-targeted therapy, as compared to an individual whose cancer does not comprise a RET fusion nucleic acid molecule or a RET fusion polypeptide. In some embodiments, responsive to the acquisition of knowledge of the RET fusion nucleic acid molecule or the RET fusion polypeptide in a sample from the individual, the individual is predicted to have increased risk of cancer recurrence, aggressive cancer, anti-cancer therapy resistance, increased RET expression, clinical benefit from a RET-targeted therapy, or poor prognosis, as compared to an individual whose cancer does not comprise a RET fusion nucleic acid molecule or a RET fusion polypeptide.

In some embodiments of any of the methods provided herein, the methods further comprise detecting or acquiring knowledge of the presence or absence of a cancer in a sample from the individual. In some embodiments, the methods comprise detecting or acquiring knowledge of the presence or absence of a cancer in a sample from the individual; and detecting or acquiring knowledge of the presence or absence of a RET fusion nucleic acid molecule, or a RET fusion polypeptide, in a sample from the individual. In some embodiments, the cancer and the RET fusion nucleic acid molecule, or the RET fusion polypeptide, are detected, or knowledge thereof is acquired, in the same sample or in different samples.

In some embodiments, responsive to acquisition of knowledge or detection of a RET fusion nucleic acid molecule or a RET fusion polypeptide of the disclosure in a sample from an individual (e.g., an individual having cancer, suspected of having cancer, being tested for cancer, or being treated for cancer), the methods comprise administering to the individual an effective amount of a treatment that comprises an anti-cancer therapy, such as an anti-cancer therapy provided herein, e.g., a RET-targeted therapy.

In some embodiments of any of the methods provided herein, the methods further comprise generating a report comprising one or more treatment options identified for the individual based, at least in part, on knowledge of a RET fusion nucleic acid molecule or a RET fusion polypeptide of the disclosure in a sample from the individual, wherein the one or more treatment options comprise an anti-cancer therapy, such as an anti-cancer therapy provided herein, e.g., a RET-targeted therapy.

In some embodiments, acquiring knowledge of a RET fusion nucleic acid molecule or a RET fusion polypeptide of the disclosure in a sample comprises detecting the RET fusion nucleic acid molecule or polypeptide in the sample. In some embodiments of any of the methods provided herein, detecting a RET fusion nucleic acid molecule of the disclosure comprises detecting a fragment of the fusion nucleic acid molecule comprising a breakpoint or fusion junction, e.g., one or more of the corresponding breakpoints described herein. In some embodiments of any of the methods provided herein, detecting a RET fusion polypeptide of the disclosure comprises detecting a portion of the fusion polypeptide that is encoded by a fragment of a RET fusion nucleic acid molecule that comprises a breakpoint or a fusion junction, e.g., one or more of the corresponding breakpoints described herein. In some embodiments, the fragment comprises any of at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more, nucleotides in length. In some embodiments, the fragment comprises between about 5 and about 100 nucleotides, between about 10 and about 50 nucleotides, or between about 10 and about 20 nucleotides, including any specific value within each of the recited ranges. In some embodiments, the fragment comprises any of at least about 1, 2, 3, 4, 5, 6, 7, 8, 9, or 10 nucleotides on either side of the breakpoint or fusion junction, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more, nucleotides on either side of the breakpoint or fusion junction. In some embodiments of any of the methods provided herein, detecting a RET fusion polypeptide of the disclosure comprises detecting a portion of the fusion polypeptide that comprises the fusion between the RET polypeptide, or the portion thereof, and the polypeptide encoded by the other gene in the fusion, or the portion thereof. In some embodiments, the portion comprises any of at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more, amino acids in length. In some embodiments, the portion comprises between about 5 and about 100 amino acids, between about 10 and about 50 amino acids, or between about 10 and about 20 amino acids, including any specific value within each of the recited ranges. In some embodiments, the portion comprises any of at least about 1, 2, 3, 4, 5, 6, 7, 8, 9, or 10 amino acids on either side of the junction, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more, amino acids on either side of the fusion junction.

In some embodiments, the methods of the disclosure further comprise providing an assessment of the RET fusion nucleic acid molecule or the RET fusion polypeptide of the disclosure in the sample.

In some embodiments of any of the methods provided herein, the anti-cancer therapy, the treatment, or treatment options comprise a RET-targeted therapy. In some embodiments, the RET-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROtcolysis-TArgeting Chimera (PROTAC), a treatment for cancer comprising a RET fusion nucleic acid molecule or polypeptide, a treatment for RET-positive or RET-rearranged cancer, a RET-targeted therapy being tested in a clinical trial, a treatment for cancer being tested in a clinical trial, a targeted therapy, a treatment being tested in a clinical trial for cancer comprising a RET fusion nucleic acid molecule or polypeptide, or any combination thereof, e.g., a described in further detail below. In some embodiments, the RET-targeted therapy is a kinase inhibitor, such as a kinase inhibitor described herein or known in the art. In some embodiments, the RET-targeted therapy is a tyrosine kinase inhibitor described herein or known in the art. In some embodiments, the RET-targeted therapy is a kinase inhibitor that inhibits the kinase activity of a RET polypeptide. In some embodiments, the RET-targeted therapy is a multi-kinase inhibitor or a RET-specific inhibitor. In some embodiments, the RET-targeted therapy comprises one or more of pralsctinib, selpercatinib, lenvatinib, sorafenib, sunitinib, vandetanib, NVP-AST487, regorafenib, dovitinib, motesanib, cabozantinib, lapatinib, lestaurtinib, linifanib, semaxinib, ponatinib, fostamatinib, quizartinib, imatinib, vatalanib, ENMD-2076, JNJ-26483327, DCC-2157, Zetelctinib, TPX0046, TAS0953, RXDX-105, LOXO-260, BOS172738, Alectinib, APS03118, LOX-18228, or SYHA1815. In some embodiments, the RET-targeted therapy inhibits the expression of a RET fusion nucleic acid molecule or polypeptide of the disclosure. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the nucleic acid inhibits the expression of a RET fusion nucleic acid molecule or polypeptide of the disclosure. In some embodiments, the nucleic acid comprises a double-stranded RNA (dsRNA), a small interfering RNA (siRNA), or a small hairpin RNA (shRNA), e.g., as described herein. In some embodiments, the RET-targeted therapy is a first-line or front-line treatment for cancer.

In some embodiments of any of the methods provided herein, the methods further comprise acquiring knowledge of or detecting in a sample from the individual a base substitution, a short insertion/deletion (indel), a copy number alteration, or a genomic rearrangement in one or more genes. In some embodiments, the one or more genes comprise one or more known/suspected oncogenes and/or tumor suppressors, one or more cancer-related genes, or any combination thereof. Alternatively or additionally, in some embodiments, the one or more genes comprise one or more of ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKNIA, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNFIA, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MSTIR, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2RIA, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSCIL1, WT1, XPO1, XRCC2, ZNF217, ZNF703, or any combination thereof. Alternatively or additionally, in some embodiments, the one or more genes comprise one or more of ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1B. IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3K8, PIGF, PTCH, RAF, RANKL, RET, ROS1, SLAMF7, VEGF, VEGFA, VEGFB, or any combination thereof. Alternatively or additionally, in some embodiments, the one or more genes comprise one or more of CPEB3, OPTN, GRM7, AGBL4, PPP2R5A, OXR1, LRMDA, MGEA5, ADCY1, SAMD4A, HSD17B7P2, ACPP, ZNF248, MKX, MYH14, BMS1, GOLGB1, NTRK2, ABI3BP, ARHGAP19, GP2, SH2D3A, VSTM4, RBMS3, LINC00379, ZNF721, ZSWIM6, SGIP1, DCC, CCNY, WDFY4, EEA1, ELMO1, RAD1, NAALADL2, CCBE1, CEP135, RAI14, ADAMTS14, SORBS1, CSGALNACT2, PCM1, KIAA1217, MPRIP, TFG, SPECC1L, REEP3, RRBP1, ETV6, TAF3, RASGEF1A, TNIP2, SATB1, ALOX5, ANKRD26, CLIP1, DLG5, ERC1, FRMD4A, KIAA1468, NCOA4, PARD3, PRKAR1A, PRKG1, RUFY2, SNRNP70, SQSTM1, TNIP1, TRIM27, TRIM33, CCDC6, TRIM24, FGFR1OP, GAS2, HOOK1, LMNA, MLPH, MYH9, PCDH15, PIBF1, SLC12A2, or ZNF485, or any gene listed in Tables 1-2, and any combination thereof.

In some embodiments of any of the methods provided herein, the treatment or the one or more treatment options, e.g., the RET-targeted therapy, further comprise an additional anti-cancer therapy, e.g., a RET-targeted therapy in combination with an additional anti-cancer therapy. In some embodiments of any of the methods provided herein, the treatment or the one or more treatment options, e.g., the RET-targeted therapy, further comprise administering an additional anti-cancer therapy to the individual, e.g., administering a RET-targeted therapy in combination with an additional anti-cancer therapy. In some embodiments, the additional anti-cancer therapy is any anti-cancer therapy known in the art or described herein. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROtcolysis-TArgeting Chimera (PROTAC), or any combination thereof. In some embodiments of any of the methods provided herein, the additional anti-cancer therapy is selected based on the presence or absence of an alteration (e.g., a base substitution, a short insertion/deletion (indel), a copy number alteration, or a genomic rearrangement) in one or more genes, e.g., one or more genes as described above.

In some embodiments, the individual has been previously treated, or is being treated, for cancer with a treatment for cancer, e.g., an anti-cancer therapy described herein or any other anti-cancer therapy or treatment known in the art. In some embodiments, the individual has been previously treated, or is being treated, for cancer with a kinase inhibitor. In some embodiments, a RET fusion nucleic acid molecule and/or a RET fusion polypeptide of the disclosure confer resistance of a cancer to a treatment for cancer, e.g., a prior treatment for cancer. In some embodiments, the cancer progressed on a prior treatment, such as a kinase inhibitor. In some embodiments, the cancer is refractory to a prior anti-cancer therapy, such as a prior kinase inhibitor therapy. In some embodiments, the cancer progressed on a prior treatment with a chemotherapy and a kinase inhibitor. In some embodiments, the individual has not been previously treated for cancer. In some embodiments, the individual, or the cancer, has not been previously treated with a kinase inhibitor. In some embodiments, the individual, or the cancer, is kinase inhibitor naïve.

In some embodiments of any of the methods provided herein, the cancer is a carcinoma, a sarcoma, a lymphoma, a leukemia, a myeloma, a germ cell cancer, or a blastoma. In some embodiments, the cancer is a solid tumor. In some embodiments, the cancer is a hematologic malignancy. In some embodiments, the cancer is a lymphoma. In some embodiments, the cancer is an ovarian cancer, a thyroid cancer, an adenocarcinoma, a breast cancer, a lung cancer, a colon cancer, a carcinoma, a uterine cancer, a prostate cancer, a pancreatic cancer, a leiomyosarcoma, a sarcoma, an esophageal cancer, a brain cancer, a bladder cancer, a skin cancer, a cervical cancer, or a melanoma. In some embodiments, the cancer is ovary epithelial carcinoma, thyroid papillary carcinoma, unknown primary adenocarcinoma, breast carcinoma, lung non-small cell lung carcinoma, colon adenocarcinoma, unknown primary carcinoma, breast invasive ductal carcinoma, uterus endometrial adenocarcinoma mixed histology, prostate acinar adenocarcinoma, lung squamous cell carcinoma, lung small cell undifferentiated carcinoma, pancreas ductal adenocarcinoma, bladder urothelial (transitional cell) carcinoma, soft tissue leiomyosarcoma, soft tissue sarcoma, esophagus adenocarcinoma, ovary serous carcinoma, colon neuroendocrine carcinoma, brain glioblastoma, breast carcinoma, unknown primary malignant neoplasm, lung adenocarcinoma, unknown primary cancer, unknown primary serous carcinoma, thyroid carcinoma, uterus carcinosarcoma, pancreatobiliary carcinoma, unknown primary urothelial carcinoma, neuroendocrine tumor, unknown primary neuroendocrine tumor, brain astrocytoma, cholangiocarcinoma, intra-hepatic cholangiocarcinoma, cervix squamous cell carcinoma, or unknown primary melanoma. In some embodiments, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pincaloma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypercosinophilia, chronic cosinophilic leukemia, neuroendocrine cancers, or a carcinoid tumor. In some embodiments, the cancer is acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some embodiments, any cancer known in the art, or any of the cancers described herein, comprise any of the RET fusion nucleic acid molecules of the disclosure, e.g., a RET fusion nucleic acid molecule described above and/or in the Examples herein. In other embodiments, any cancer known in the art, or any of the cancers described herein, comprise any of the RET fusion polypeptides of the disclosure, e.g., a RET fusion polypeptide described above and/or in the Examples herein. In some embodiments, the methods provided herein comprise acquiring knowledge of or detecting any of the RET fusion nucleic acid molecules or RET fusion polypeptides of the disclosure in a sample from an individual having, suspected of having, being tested for, or being treated for any cancer known in the art, or any of the cancers described herein.

In some embodiments, the cancer is any cancer known in the art or described herein, and the RET fusion nucleic acid molecule, or the RET fusion polypeptide, is any of the GOLGB1-RET, GP2-RET, PPP2R5A-RET, GRM7-RET, MYH14-RET, MGEA5-RET, SAMD4A-RET, ACPP-RET, BMS1-RET, CEP135-RET, EEA1-RET, RET-ADCY1, RET-ZNF248, RET-AGBL4, RET-LRMDA, RET-ARHGAP19, RET-CPEB3, RET-DCC, RET-ELMO1, RET-WDFY4, MKX-RET, RBMS3-RET, SGIP1-RET, ZSWIM6-RET, RET-NTRK2, RET-OXR1, RET-CCBE1, RET-NAALADL2, RAI14-RET, ABI3BP-RET, LINC00379-RET, OPTN-RET, SH2D3A-RET, ZNF721-RET, ADAMTS14-RET, CPEB3-RET, RET-CCNY, RET-HSD17B7P2, RET-RAD1, RET-VSTM4, ANKRD26-RET, CLIP1-RET, DLG5-RET, ERC1-RET, FRMD4A-RET, KIAA1468-RET, NCOA4-RET, PARD3-RET, PRKAR1A-RET, PRKG1-RET, RUFY2-RET, SNRNP70-RET, SQSTM1-RET.

TNIP1-RET, TRIM27-RET, TRIM33-RET, CCDC6-RET, TRIM24-RET, FGFR1OP-RET, GAS2-RET, HOOK1-RET, LMNA-RET, MLPH-RET, MYH9-RET, PCDH15-RET, PIBF1-RET, SLC12A2-RET, or ZNF485-RET fusion nucleic acid molecules or polypeptides described herein. e.g., above and/or in the Examples herein. In some embodiments, the cancer is a cancer listed in Table 10, and the RET fusion nucleic acid molecule (or the encoded RET fusion polypeptide) is the corresponding RET fusion nucleic acid molecule (or encoded RET fusion polypeptide) as listed in Table 10.

TABLE 10
Exemplary RET fusion nucleic acid molecules
and corresponding cancer types.

| RET Fusion Nucleic | |
|---|---|
| Acid Molecule | Cancer Type |
| GOLGB1-RET | Thyroid papillary carcinoma |
| GP2-RET | Unknown primary adenocarcinoma |
| PPP2R5A-RET | Breast carcinoma |
| GRM7-RET | Colon adenocarcinoma |
| MYH14-RET | Breast invasive ductal carcinoma |
| MGEA5-RET | Ovary epithelial carcinoma |
| SAMD4A-RET | Uterus endometrial adenocarcinoma mixed |
| | histology |
| ACPP-RET | Prostate acinar adenocarcinoma |
| BMS1-RET | Lung squamous cell carcinoma |
| CEP135-RET | Lung small cell undifferentiated carcinoma |
| EEA1-RET | Pancreas ductal adenocarcinoma |
| RET-ADCY1 | Breast carcinoma |
| RET-ZNF248 | Unknown primary malignant neoplasm |
| RET-AGBL4 | Ovary serous carcinoma |
| RET-LRMDA | Ovary serous carcinoma |
| RET-ARHGAP19 | Lung adenocarcinoma |
| RET-CPEB3 | Lung adenocarcinoma |
| RET-DCC | Lung adenocarcinoma |
| RET-ELMO1 | Lung adenocarcinoma |
| RET-WDFY4 | Esophagus adenocarcinoma |
| MKX-RET | Lung adenocarcinoma |
| RBMS3-RET | Uterus endometrial adenocarcinoma |
| SGIP1-RET | Lung adenocarcinoma |
| ZSWIM6-RET | Lung adenocarcinoma |
| RET-NTRK2 | Bladder urothelial (transitional cell) carcinoma |
| RET-OXR1 | Unknown primary serous carcinoma |
| RET-CCBE1 | Lung adenocarcinoma |
| RET-NAALADL2 | Thyroid papillary carcinoma |
| RAI14-RET | Lung adenocarcinoma |
| ABI3BP-RET | Thyroid carcinoma |
| LINC00379-RET | Breast carcinoma |
| OPTN-RET | Lung adenocarcinoma |
| SH2D3A-RET | Lung non-small cell lung carcinoma |
| ZNF721-RET | Lung non-small cell lung carcinoma |
| ADAMTS14-RET | Esophagus adenocarcinoma |
| CPEB3-RET | Lung adenocarcinoma |
| RET-CCNY | Breast invasive ductal carcinoma |
| RET-HSD17B7P2 | Unknown primary carcinoma |
| RET-RAD1 | Lung adenocarcinoma |
| RET-VSTM4 | Bladder urothelial (transitional cell) carcinoma |
| FGFR1OP-RET | Unknown primary adenocarcinoma |
| GAS2-RET | Lung non-small cell lung carcinoma |
| HOOK1-RET | Intra-hepatic cholangiocarcinoma |
| LMNA-RET | Thyroid papillary carcinoma |
| MLPH-RET | Unknown primary melanoma |
| MYH9-RET | Lung non-small cell lung carcinoma |
| PCDH15-RET | Lung adenocarcinoma |
| PIBF1-RET | Lung adenocarcinoma |
| SLC12A2-RET | Lung adenocarcinoma |
| ZNF485-RET | Cervix squamous cell carcinoma |
| ZNF485-RET | Lung adenocarcinoma |

In some embodiments, the cancer is thyroid papillary carcinoma, and the RET fusion nucleic acid molecule, or the RET fusion polypeptide, is a GOLGB1-RET fusion nucleic acid molecule or polypeptide described herein (e.g., above and/or in the Examples herein). In some embodiments, the cancer is unknown primary adenocarcinoma, and the RET fusion nucleic acid molecule, or the RET fusion polypeptide, is a GP2-RET fusion nucleic acid molecule or polypeptide described herein (e.g., above and/or in the Examples herein). In some embodiments, the cancer is breast carcinoma, and the RET fusion nucleic acid molecule, or the RET fusion polypeptide, is a PPP2R5A-RET fusion nucleic acid molecule or polypeptide described herein (e.g., above and/or in the Examples herein). In some embodiments, the cancer is colon adenocarcinoma, and the RET fusion nucleic acid molecule, or the RET fusion polypeptide, is a GRM7-RET fusion nucleic acid molecule or polypeptide described herein (e.g., above and/or in the Examples herein). In some embodiments, the cancer is breast invasive ductal carcinoma, and the RET fusion nucleic acid molecule, or the RET fusion polypeptide, is a MYH14-RET fusion nucleic acid molecule or polypeptide described herein (e.g., above and/or in the Examples herein). In some embodiments, the cancer is ovary epithelial carcinoma, and the RET fusion nucleic acid molecule, or the RET fusion polypeptide, is a MGEA5-RET fusion nucleic acid molecule or polypeptide described herein (e.g., above and/or in the Examples herein). In some embodiments, the cancer is uterus endometrial adenocarcinoma mixed histology, and the RET fusion nucleic acid molecule, or the RET fusion polypeptide, is a SAMD4A-RET fusion nucleic acid molecule or polypeptide described herein (e.g., above and/or in the Examples herein). In some embodiments, the cancer is prostate acinar adenocarcinoma, and the RET fusion nucleic acid molecule, or the RET fusion polypeptide, is a ACPP-RET fusion nucleic acid molecule or polypeptide described herein (e.g., above and/or in the Examples herein). In some embodiments, the cancer is lung squamous cell carcinoma, and the RET fusion nucleic acid molecule, or the RET fusion polypeptide, is a BMS1-RET fusion nucleic acid molecule or polypeptide described herein (e.g., above and/or in the Examples herein). In some embodiments, the cancer is lung small cell undifferentiated carcinoma, and the RET fusion nucleic acid molecule, or the RET fusion polypeptide, is a CEP135-RET fusion nucleic acid molecule or polypeptide described herein (e.g., above and/or in the Examples herein). In some embodiments, the cancer is pancreas ductal adenocarcinoma, and the RET fusion nucleic acid molecule, or the RET fusion polypeptide, is a EEA1-RET fusion nucleic acid molecule or polypeptide described herein (e.g., above and/or in the Examples herein). In some embodiments, the cancer is breast carcinoma, and the RET fusion nucleic acid molecule, or the RET fusion polypeptide, is a RET-ADCY1 fusion nucleic acid molecule or polypeptide described herein (e.g., above and/or in the Examples herein). In some embodiments, the cancer is unknown primary malignant neoplasm, and the RET fusion nucleic acid molecule, or the RET fusion polypeptide, is a RET-ZNF248 fusion nucleic acid molecule or polypeptide described herein (e.g., above and/or in the Examples herein). In some embodiments, the cancer is ovary serous carcinoma, and the RET fusion nucleic acid molecule, or the RET fusion polypeptide, is a RET-AGBL4 fusion nucleic acid molecule or polypeptide described herein (e.g., above and/or in the Examples herein). In some embodiments, the cancer is ovary serous carcinoma, and the RET fusion nucleic acid molecule, or the RET fusion polypeptide, is a RET-LRMDA fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a RET-ARHGAP19 fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a RET-CPEB3 fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a RET-DCC fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a RET-ELMO1 fusion nucleic acid molecule or polypeptide described herein (e.g., above and/or in the Examples herein). In some embodiments, the cancer is esophagus adenocarcinoma, and the RET fusion nucleic acid molecule, or the RET fusion polypeptide, is a RET-WDFY4 fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a MKX-RET fusion nucleic acid molecule or polypeptide described herein (e.g., above and/or in the Examples herein). In some embodiments, the cancer is uterus endometrial adenocarcinoma, and the RET fusion nucleic acid molecule, or the RET fusion polypeptide, is a RBMS3-RET fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a SGIP1-RET fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a ZSWIM6-RET fusion nucleic acid molecule or polypeptide described herein (e.g., above and/or in the Examples herein). In some embodiments, the cancer is bladder urothelial (transitional cell) carcinoma, and the RET fusion nucleic acid molecule, or the RET fusion polypeptide, is a RET-NTRK2 fusion nucleic acid molecule or polypeptide described herein (e.g., above and/or in the Examples herein). In some embodiments, the cancer is unknown primary serous carcinoma, and the RET fusion nucleic acid molecule, or the RET fusion polypeptide, is a RET-OXR1 fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a RET-CCBE1 fusion nucleic acid molecule or polypeptide described herein (e.g., above and/or in the Examples herein). In some embodiments, the cancer is thyroid papillary carcinoma, and the RET fusion nucleic acid molecule, or the RET fusion polypeptide, is a RET-NAALADL2 fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a RAI14-RET fusion nucleic acid molecule or polypeptide described herein (e.g., above and/or in the Examples herein). In some embodiments, the cancer is thyroid carcinoma, and the RET fusion nucleic acid molecule, or the RET fusion polypeptide, is a ABI3BP-RET fusion nucleic acid molecule or polypeptide described herein (e.g., above and/or in the Examples herein). In some embodiments, the cancer is breast carcinoma, and the RET fusion nucleic acid molecule, or the RET fusion polypeptide, is a LINC00379-RET fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a OPTN-RET fusion nucleic acid molecule or polypeptide described herein (e.g., above and/or in the Examples herein). In some embodiments, the cancer is lung non-small cell lung carcinoma, and the RET fusion nucleic acid molecule, or the RET fusion polypeptide, is a SH2D3A-RET fusion nucleic acid molecule or polypeptide described herein (e.g., above and/or in the Examples herein). In some embodiments, the cancer is lung non-small cell lung carcinoma, and the RET fusion nucleic acid molecule, or the RET fusion polypeptide, is a ZNF721-RET fusion nucleic acid molecule or polypeptide described herein (e.g., above and/or in the Examples herein). In some embodiments, the cancer is esophagus adenocarcinoma, and the RET fusion nucleic acid molecule, or the RET fusion polypeptide, is a ADAMTS14-RET fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a CPEB3-RET fusion nucleic acid molecule or polypeptide described herein (e.g., above and/or in the Examples herein). In some embodiments, the cancer is breast invasive ductal carcinoma, and the RET fusion nucleic acid molecule, or the RET fusion polypeptide, is a RET-CCNY fusion nucleic acid molecule or polypeptide described herein (e.g., above and/or in the Examples herein). In some embodiments, the cancer is unknown primary carcinoma, and the RET fusion nucleic acid molecule, or the RET fusion polypeptide, is a RET-HSD17B7P2 fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a RET-RAD1 fusion nucleic acid molecule or polypeptide described herein (e.g., above and/or in the Examples herein). In some embodiments, the cancer is bladder urothelial (transitional cell) carcinoma, and the RET fusion nucleic acid molecule, or the RET fusion polypeptide, is a RET-VSTM4 fusion nucleic acid molecule or polypeptide described herein (e.g., above and/or in the Examples herein). In some embodiments, the cancer is unknown primary adenocarcinoma, and the RET fusion nucleic acid molecule, or the RET fusion polypeptide, is a FGFR1OP-RET fusion nucleic acid molecule or polypeptide described herein (e.g., above and/or in the Examples herein). In some embodiments, the cancer is lung non-small cell lung carcinoma, and the RET fusion nucleic acid molecule, or the RET fusion polypeptide, is a GAS2-RET fusion nucleic acid molecule or polypeptide described herein (e.g., above and/or in the Examples herein). In some embodiments, the cancer is intra-hepatic cholangiocarcinoma, and the RET fusion nucleic acid molecule, or the RET fusion polypeptide, is a HOOK1-RET fusion nucleic acid molecule or polypeptide described herein (e.g., above and/or in the Examples herein). In some embodiments, the cancer is thyroid papillary carcinoma, and the RET fusion nucleic acid molecule, or the RET fusion polypeptide, is a LMNA-RET fusion nucleic acid molecule or polypeptide described herein (e.g., above and/or in the Examples herein). In some embodiments, the cancer is unknown primary melanoma, and the RET fusion nucleic acid molecule, or the RET fusion polypeptide, is a MLPH-RET fusion nucleic acid molecule or polypeptide described herein (e.g., above and/or in the Examples herein). In some embodiments, the cancer is lung non-small cell lung carcinoma, and the RET fusion nucleic acid molecule, or the RET fusion polypeptide, is a MYH9-RET fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a PCDH15-RET fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a PIBF1-RET fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a SLC12A2-RET fusion nucleic acid molecule or polypeptide described herein (e.g., above and/or in the Examples herein). In some embodiments, the cancer is cervix squamous cell carcinoma, and the RET fusion nucleic acid molecule, or the RET fusion polypeptide, is a ZNF485-RET fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a ZNF485-RET fusion nucleic acid molecule or polypeptide described herein (e.g., above and/or in the Examples herein).

In some embodiments, the cancer is a cancer listed in Table 2, and the RET fusion nucleic acid molecule (or the encoded RET fusion polypeptide) is the corresponding RET fusion nucleic acid molecule (or encoded RET fusion polypeptide) as listed in Table 2. In some embodiments, the cancer is soft tissue leiomyosarcoma, and the RET fusion nucleic acid molecule, or the RET fusion polypeptide, is a SORBS1-RET fusion nucleic acid molecule or polypeptide described herein (e.g., above and/or in the Examples herein). In some embodiments, the cancer is uterus carcinosarcoma, and the RET fusion nucleic acid molecule, or the RET fusion polypeptide, is a CSGALNACT2-RET fusion nucleic acid molecule or polypeptide described herein (e.g., above and/or in the Examples herein). In some embodiments, the cancer is bladder urothelial (transitional cell) carcinoma, and the RET fusion nucleic acid molecule, or the RET fusion polypeptide, is a CSGALNACT2-RET fusion nucleic acid molecule or polypeptide described herein (e.g., above and/or in the Examples herein). In some embodiments, the cancer is brain glioblastoma (GBM), and the RET fusion nucleic acid molecule, or the RET fusion polypeptide, is a PCM1-RET fusion nucleic acid molecule or polypeptide described herein (e.g., above and/or in the Examples herein). In some embodiments, the cancer is pancreas ductal adenocarcinoma, and the RET fusion nucleic acid molecule, or the RET fusion polypeptide, is a PCM1-RET fusion nucleic acid molecule or polypeptide described herein (e.g., above and/or in the Examples herein). In some embodiments, the cancer is pancreas ductal adenocarcinoma, and the RET fusion nucleic acid molecule, or the RET fusion polypeptide, is a KIAA1217-RET fusion nucleic acid molecule or polypeptide described herein (e.g., above and/or in the Examples herein). In some embodiments, the cancer is soft tissue sarcoma (NOS), and the RET fusion nucleic acid molecule, or the RET fusion polypeptide, is a MPRIP-RET fusion nucleic acid molecule or polypeptide described herein (e.g., above and/or in the Examples herein). In some embodiments, the cancer is pancreas ductal adenocarcinoma, and the RET fusion nucleic acid molecule, or the RET fusion polypeptide, is a TFG-RET fusion nucleic acid molecule or polypeptide described herein (e.g., above and/or in the Examples herein). In some embodiments, the cancer is pancreas ductal adenocarcinoma, and the RET fusion nucleic acid molecule, or the RET fusion polypeptide, is a SPECC1L-RET fusion nucleic acid molecule or polypeptide described herein (e.g., above and/or in the Examples herein). In some embodiments, the cancer is esophagus adenocarcinoma, and the RET fusion nucleic acid molecule, or the RET fusion polypeptide, is a REEP3-RET fusion nucleic acid molecule or polypeptide described herein (e.g., above and/or in the Examples herein). In some embodiments, the cancer is ovary serous carcinoma, and the RET fusion nucleic acid molecule, or the RET fusion polypeptide, is a RRBP1-RET fusion nucleic acid molecule or polypeptide described herein (e.g., above and/or in the Examples herein). In some embodiments, the cancer is pancreas ductal adenocarcinoma, and the RET fusion nucleic acid molecule, or the RET fusion polypeptide, is a ETV6-RET fusion nucleic acid molecule or polypeptide described herein (e.g., above and/or in the Examples herein). In some embodiments, the cancer is colon neuroendocrine carcinoma, and the RET fusion nucleic acid molecule, or the RET fusion polypeptide, is a TAF3-RET fusion nucleic acid molecule or polypeptide described herein (e.g., above and/or in the Examples herein). In some embodiments, the cancer is unknown primary melanoma, and the RET fusion nucleic acid molecule, or the RET fusion polypeptide, is a RET-RASGEF1A fusion nucleic acid molecule or polypeptide described herein (e.g., above and/or in the Examples herein). In some embodiments, the cancer is colon adenocarcinoma (CRC), and the RET fusion nucleic acid molecule, or the RET fusion polypeptide, is a TNIP2-RET fusion nucleic acid molecule or polypeptide described herein (e.g., above and/or in the Examples herein). In some embodiments, the cancer is pancreas ductal adenocarcinoma, and the RET fusion nucleic acid molecule, or the RET fusion polypeptide, is a SATB1-RET fusion nucleic acid molecule or polypeptide described herein (e.g., above and/or in the Examples herein). In some embodiments, the cancer is brain glioblastoma (GBM), and the RET fusion nucleic acid molecule, or the RET fusion polypeptide, is a ALOX5-RET fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is an ANKRD26-RET fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a CLIP1-RET fusion nucleic acid molecule or polypeptide described herein (e.g., above and/or in the Examples herein). In some embodiments, the cancer is ovary serous carcinoma, and the RET fusion nucleic acid molecule, or the RET fusion polypeptide, is a DLG5-RET fusion nucleic acid molecule or polypeptide described herein (e.g., above and/or in the Examples herein). In some embodiments, the cancer is breast carcinoma, and the RET fusion nucleic acid molecule, or the RET fusion polypeptide, is a DLG5-RET fusion nucleic acid molecule or polypeptide described herein (e.g., above and/or in the Examples herein). In some embodiments, the cancer is soft tissue sarcoma, and the RET fusion nucleic acid molecule, or the RET fusion polypeptide, is an ERC1-RET fusion nucleic acid molecule or polypeptide described herein (e.g., above and/or in the Examples herein). In some embodiments, the cancer is unknown primary carcinoma, and the RET fusion nucleic acid molecule, or the RET fusion polypeptide, is an ERC1-RET fusion nucleic acid molecule or polypeptide described herein (e.g., above and/or in the Examples herein). In some embodiments, the cancer is pancreatobiliary carcinoma, and the RET fusion nucleic acid molecule, or the RET fusion polypeptide, is an ERC1-RET fusion nucleic acid molecule or polypeptide described herein (e.g., above and/or in the Examples herein). In some embodiments, the cancer is unknown primary adenocarcinoma, and the RET fusion nucleic acid molecule, or the RET fusion polypeptide, is a FRMD4A-RET fusion nucleic acid molecule or polypeptide described herein (e.g., above and/or in the Examples herein). In some embodiments, the cancer is thyroid papillary carcinoma, and the RET fusion nucleic acid molecule, or the RET fusion polypeptide, is a KIAA1468-RET fusion nucleic acid molecule or polypeptide described herein (e.g., above and/or in the Examples herein). In some embodiments, the cancer is colon adenocarcinoma (CRC), and the RET fusion nucleic acid molecule, or the RET fusion polypeptide, is a NCOA4-RET fusion nucleic acid molecule or polypeptide described herein (e.g., above and/or in the Examples herein). In some embodiments, the cancer is unknown primary urothelial carcinoma, and the RET fusion nucleic acid molecule, or the RET fusion polypeptide, is a PARD3-RET fusion nucleic acid molecule or polypeptide described herein (e.g., above and/or in the Examples herein). In some embodiments, the cancer is unknown primary neuroendocrine tumor, and the RET fusion nucleic acid molecule, or the RET fusion polypeptide, is a PARD3-RET fusion nucleic acid molecule or polypeptide described herein (e.g., above and/or in the Examples herein). In some embodiments, the cancer is brain astrocytoma, and the RET fusion nucleic acid molecule, or the RET fusion polypeptide, is a PRKAR1A-RET fusion nucleic acid molecule or polypeptide described herein (e.g., above and/or in the Examples herein). In some embodiments, the cancer is prostate acinar adenocarcinoma, and the RET fusion nucleic acid molecule, or the RET fusion polypeptide, is a PRKG1-RET fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a PRKG1-RET fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a RUFY2-RET fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a SNRNP70-RET fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a SQSTM1-RET fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a TNIP1-RET fusion nucleic acid molecule or polypeptide described herein (e.g., above and/or in the Examples herein). In some embodiments, the cancer is colon adenocarcinoma (CRC), and the RET fusion nucleic acid molecule, or the RET fusion polypeptide, is a TRIM27-RET fusion nucleic acid molecule or polypeptide described herein (e.g., above and/or in the Examples herein). In some embodiments, the cancer is pancreas ductal adenocarcinoma, and the RET fusion nucleic acid molecule, or the RET fusion polypeptide, is a TRIM33-RET fusion nucleic acid molecule or polypeptide described herein (e.g., above and/or in the Examples herein). In some embodiments, the cancer is ovary epithelial carcinoma, and the RET fusion nucleic acid molecule, or the RET fusion polypeptide, is a CCDC6-RET fusion nucleic acid molecule or polypeptide described herein (e.g., above and/or in the Examples herein). In some embodiments, the cancer is lung adenocarcinoma, and the RET fusion nucleic acid molecule, or the RET fusion polypeptide, is a TRIM24-RET fusion nucleic acid molecule or polypeptide described herein (e.g., above and/or in the Examples herein).

In some embodiments, the cancer is metastatic.

In some embodiments of any of the methods provided herein, the sample is a sample described below. In some embodiments, the sample is obtained from the individual or from the cancer. In some embodiments, the methods further comprise obtaining the sample, e.g., from the individual or from the cancer. In some embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, cerebrospinal fluid, sputum, stool, urine, or saliva. In some embodiments, the sample comprises cells and/or nucleic acids from the cancer. In some embodiments, the sample comprises mRNA, DNA, circulating tumor DNA (ctDNA), cell-free DNA, or cell-free RNA from the cancer. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the RET fusion nucleic acid molecule or polypeptide is detected in a tissue biopsy sample, in a liquid biopsy sample, or in both a tissue biopsy sample and a liquid biopsy sample, from the individual.

### B. Detection of RET Fusion Nucleic Acid Molecules and Polypeptides

Certain aspects of the present disclosure relate to detection of a RET fusion nucleic acid molecule of the disclosure (e.g., any of the RET fusion nucleic acid molecules described above and/or in the Examples herein) e.g., in a patient sample. In some embodiments, the RET fusion nucleic acid molecule is detected in vitro.

Other aspects of the present disclosure relate to detection of a RET fusion polypeptide of the disclosure (e.g., any of the RET fusion polypeptides described above and/or in the Examples herein) e.g., in a patient sample. In some embodiments, the RET fusion polypeptide is detected in vitro.

### (i) Detection of RET Fusion Nucleic Acid Molecules

Provided herein are methods of detecting a RET fusion nucleic acid molecule of the disclosure (e.g., any of the RET fusion nucleic acid molecules described above and/or in the Examples herein), or a fragment thereof, in a sample.

Methods for detecting fusion nucleic acid molecules are known in the art. For example, in some embodiments, a RET fusion nucleic acid molecule of the disclosure may be detected by sequencing part or all of a gene involved in the fusion nucleic acid molecule, e.g., a RET gene, and/or a corresponding fusion partner gene described herein (e.g., any of CPEB3, OPTN, GRM7, AGBL4. PPP2R5A, OXR1, LRMDA, MGEA5, ADCY1, SAMD4A, HSD17B7P2, ACPP, ZNF248, MKX, MYH14, BMS1, GOLGB1, NTRK2, ABI3BP, ARHGAP19, GP2, SH2D3A, VSTM4, RBMS3, LINC00379, ZNF721, ZSWIM6, SGIP1, DCC, CCNY, WDFY4, EEA1, ELMO1, RAD1, NAALADL2, CCBE1, CEP135, RAI14, ADAMTS14, SORBS1, CSGALNACT2, PCM1, KIAA1217, MPRIP, TFG, SPECC1L, REEP3, RRBP1, ETV6, TAF3, RASGEF1A, TNIP2, SATB1, ALOX5, ANKRD26, CLIP1, DLG5, ERC1. FRMD4A, KIAA1468, NCOA4, PARD3, PRKAR1A, PRKG1, RUFY2, SNRNP70, SQSTM1, TNIP1, TRIM27, TRIM33, CCDC6, TRIM24, FGFR1OP, GAS2, HOOK1, LMNA, MLPH, MYH9, PCDH15, PIBF1, SLC12A2, or ZNF485, or any gene listed in Tables 1-2), by next-generation or other sequencing of DNA, RNA, or cDNA. In some embodiments, a RET fusion nucleic acid molecule of the disclosure is detected by PCR amplification of DNA, RNA, or cDNA. In some embodiments, a RET fusion nucleic acid molecule of the disclosure is detected by in situ hybridization using one or more polynucleotides that hybridize to a locus involved in the fusion nucleic acid molecule, e.g., a RET locus, and/or a corresponding fusion partner gene locus described herein (e.g., any of CPEB3, OPTN, GRM7, AGBL4, PPP2R5A, OXR1, LRMDA, MGEA5, ADCY1, SAMD4A, HSD17B7P2, ACPP, ZNF248, MKX, MYH14, BMS1, GOLGB1, NTRK2, ABI3BP, ARHGAP19, GP2, SH2D3A, VSTM4, RBMS3, LINC00379, ZNF721, ZSWIM6, SGIP1, DCC, CCNY, WDFY4, EEA1, ELMO1, RAD1, NAALADL2, CCBE1, CEP135, RAI14, ADAMTS14, SORBS1, CSGALNACT2, PCM1, KIAA1217, MPRIP, TFG, SPECC1L, REEP3, RRBP1, ETV6, TAF3, RASGEF1A, TNIP2, SATB1, ALOX5, ANKRD26, CLIP1, DLG5, ERC1, FRMD4A, KIAA1468, NCOA4, PARD3, PRKAR1A, PRKG1, RUFY2, SNRNP70, SQSTM1, TNIP1, TRIM27, TRIM33, CCDC6, TRIM24, FGFR1OP, GAS2, HOOK1, LMNA, MLPH, MYH9, PCDH15, PIBF1, SLC12A2, or ZNF485, or any gene listed in Tables 1-2), e.g., using fluorescence in situ hybridization (FISH). In some embodiments, a RET fusion nucleic acid molecule of the disclosure is detected in a cancer or tumor cell, e.g., using tumor tissue, such as from a tumor biopsy or other tumor specimen; in a circulating cancer or tumor cell, e.g., using a liquid biopsy, such as from blood, plasma, cerebrospinal fluid, sputum, stool, urine, or saliva; or in circulating tumor DNA (ctDNA), e.g., using a liquid biopsy, such as from blood, plasma, cerebrospinal fluid, sputum, stool, urine, or saliva.

Exemplary and non-limiting methods for detecting a RET fusion nucleic acid molecule of the disclosure are provided below.

In some embodiments, a RET fusion nucleic acid molecule of the disclosure is detected using any suitable method known in the art, such as a nucleic acid hybridization assay, an amplification-based assay (e.g., polymerase chain reaction, PCR), a PCR-RFLP assay, real-time PCR, sequencing (e.g., Sanger sequencing or next-generation sequencing), a screening analysis (e.g., using karyotype methods), fluorescence in situ hybridization (FISH), break away FISH, spectral karyotyping, multiplex-FISH, comparative genomic hybridization, in situ hybridization, single specific primer-polymerase chain reaction (SSP-PCR), high performance liquid chromatography (HPLC), or mass-spectrometric genotyping. Methods of analyzing samples, e.g., to detect a nucleic acid molecule, are described in U.S. Pat. No. 9,340,830 and in WO2012092426A1, which are hereby incorporated by reference in their entirety. In some embodiments, a RET fusion nucleic acid molecule of the disclosure is detected by sequencing. In some embodiments, the sequencing comprises a massively parallel sequencing (MPS) technique, whole genome sequencing (WGS), whole exome sequencing, targeted sequencing, direct sequencing, or a Sanger sequencing technique. In some embodiments, the massively parallel sequencing (MPS) technique comprises next-generation sequencing (NGS).

In some embodiments, a RET fusion nucleic acid molecule of the disclosure is detected using an in situ hybridization method, such as a fluorescence in situ hybridization (FISH) method.

In some embodiments, FISH analysis is used to identify the chromosomal rearrangement resulting in a RET fusion nucleic acid molecule as described herein. In some embodiments, FISH analysis is used to identify an RNA molecule comprising or encoding a RET fusion nucleic acid molecule of the disclosure. Methods for performing FISH are known in the art and can be used in nearly any type of tissue. In FISH analysis, nucleic acid probes which are detectably labeled, e.g. fluorescently labeled, are allowed to bind to specific regions of DNA, e.g., a chromosome, or an RNA, e.g., an mRNA, and then examined, e.g., through a microscope. See, for example, U.S. Pat. No. 5,776,688. DNA or RNA molecules are first fixed onto a slide, the labeled probe is then hybridized to the DNA or RNA molecules, and then visualization is achieved, e.g., using enzyme-linked label-based detection methods known in the art. Generally, the resolution of FISH analysis is on the order of detection of 60 to 100000 nucleotides, e.g., 60 base pairs (bp) up to 100 kilobase pairs of DNA. Nucleic acid probes used in FISH analysis comprise single stranded nucleic acids. Such probes are typically at least about 50 nucleotides in length. In some embodiments, probes comprise about 100 to about 500 nucleotides. Probes that hybridize with centromeric DNA and locus-specific DNA or RNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA or other sources of nucleic acids through standard techniques. Examples of probes, labeling and hybridization methods are known in the art.

Several variations of FISH methods are known in the art and are suitable for use according to the methods of the disclosure, including single-molecule RNA FISH, Fiber FISH, Q-FISH, Flow-FISH, MA-FISH, break-away FISH, hybrid fusion-FISH, and multi-fluor FISH or mFISH. In some embodiments. “break-away FISH” is used in the methods provided herein. In break-away FISH, at least one probe targeting a fusion junction or breakpoint and at least one probe targeting an individual gene of the fusion, e.g., at one or more exons and or introns of the gene, are utilized. In normal cells (i.e., cells not having a fusion nucleic acid molecule described herein), both probes are observed (or a secondary color is observed due to the close proximity of the two genes of the gene fusion); and in cells having a fusion nucleic acid molecule described herein, only a single gene probe is observed due to the presence of a rearrangement resulting in the fusion nucleic acid molecule.

In some embodiments, a RET fusion nucleic acid molecule of the disclosure is detected using an array-based method, such as array-based comparative genomic hybridization (CGH) methods. In array-based CGH methods, a first sample of nucleic acids (e.g., from a sample, such as from a tumor, or a tissue or liquid biopsy) is labeled with a first label, while a second sample of nucleic acids (e.g., a control, such as from a healthy cell/tissue) is labeled with a second label. In some embodiments, equal quantities of the two samples are mixed and co-hybridized to a DNA microarray of several thousand evenly spaced cloned DNA fragments or oligonucleotides, which have been spotted in triplicate on the array. After hybridization, digital imaging systems are used to capture and quantify the relative fluorescence intensities of each of the hybridized fluorophores. The resulting ratio of the fluorescence intensities is proportional to the ratio of the copy numbers of DNA sequences in the two samples. In some embodiments, where there are chromosomal deletions or multiplications, differences in the ratio of the signals from the two labels are detected and the ratio provides a measure of the copy number. Array-based CGH can also be performed with single-color labeling. In single color CGH, a control (e.g., control nucleic acid sample, such as from a healthy cell/tissue) is labeled and hybridized to one array and absolute signals are read, and a test sample (e.g., a nucleic acid sample obtained from an individual or from a tumor, or a tissue or liquid biopsy) is labeled and hybridized to a second array (with identical content) and absolute signals are read. Copy number differences are calculated based on absolute signals from the two arrays.

In some embodiments, a RET fusion nucleic acid molecule of the disclosure is detected using an amplification-based method. As is known in the art, in such amplification-based methods, a sample of nucleic acids, such as a sample obtained from an individual, a tumor or a tissue or liquid biopsy, is used as a template in an amplification reaction (e.g., Polymerase Chain Reaction (PCR)) using one or more oligonucleotides or primers, e.g., such as one or more oligonucleotides or primers provided herein. The presence of a fusion nucleic acid molecule of the disclosure in the sample can be determined based on the presence or absence of an amplification product. Quantitative amplification methods are also known in the art and may be used according to the methods provided herein. Methods of measurement of DNA copy number at microsatellite loci using quantitative PCR analysis are known in the art. The known nucleotide sequence for genes is sufficient to enable one of skill in the art to routinely select primers to amplify any portion of the gene. Fluorogenic quantitative PCR can also be used. In fluorogenic quantitative PCR, quantitation is based on the amount of fluorescence signals, e.g., TaqMan and Sybr green.

Other amplification methods suitable for use according to the methods provided herein include, e.g., ligase chain reaction (LCR), transcription amplification, self-sustained sequence replication, dot PCR, and linker adapter PCR.

In some embodiments, a RET fusion nucleic acid molecule of the disclosure is detected using a sequencing method. Any method of sequencing known in the art can be used to detect a RET fusion nucleic acid molecule provided herein. Exemplary sequencing methods that may be used to detect a fusion nucleic acid molecule provided herein include those based on techniques developed by Maxam and Gilbert or Sanger. Automated sequencing procedures may also be used, e.g., including sequencing by mass spectrometry.

In some embodiments, a RET fusion nucleic acid molecule of the disclosure is detected using hybrid capture-based sequencing (hybrid capture-based NGS), e.g., using adaptor ligation-based libraries. See, e.g., Frampton, G. M, et al. (2013) Nat. Biotech. 31:1023-1031, which is hereby incorporated by reference. In some embodiments, a RET fusion nucleic acid molecule of the disclosure is detected using next-generation sequencing (NGS). Next-generation sequencing includes any sequencing method that determines the nucleotide sequence of either individual nucleic acid molecules or clonally expanded proxies for individual nucleic acid molecules in a highly parallel fashion (e.g., greater than 10⁵ molecules may be sequenced simultaneously). Next generation sequencing methods suitable for use according to the methods provided herein are known in the art and include, without limitation, massively parallel short-read sequencing, template-based sequencing, pyrosequencing, real-time sequencing comprising imaging the continuous incorporation of dye-labeling nucleotides during DNA synthesis, nanopore sequencing, sequencing by hybridization, nano-transistor array based sequencing, polony sequencing, scanning tunneling microscopy (STM)-based sequencing, or nanowire-molecule sensor based sequencing. See, e.g., Metzker, M. (2010) Nature Biotechnology Reviews 11:31-46, which is hereby incorporated by reference. Exemplary NGS methods and platforms that may be used to detect a RET fusion nucleic acid molecule provided herein include, without limitation, the HeliScope Gene Sequencing system from Helicos BioSciences (Cambridge, MA., USA), the PacBio RS system from Pacific Biosciences (Menlo Park, CA, USA), massively parallel short-read sequencing such as the Solexa sequencer and other methods and platforms from Illumina Inc. (San Diego, CA, USA), 454 sequencing from 454 LifeSciences (Branford, CT, USA), Ion Torrent sequencing from ThermoFisher (Waltham, MA, USA), or the SOLID sequencer from Applied Biosystems (Foster City, CA, USA). Additional exemplary methods and platforms that may be used to detect a RET fusion nucleic acid molecule provided herein include, without limitation, the Genome Sequencer (GS) FLX System from Roche (Basel, CHE), the G.007 polonator system, the Solexa Genome Analyzer, HiSeq 2500, HiSeq3000, HiSeq 4000, and NovaSeq 6000 platforms from Illumina Inc. (San Diego, CA, USA).

In some embodiments of any of the methods provided herein, the methods may comprise one or more of the steps of: (i) obtaining a sample from an individual (e.g., an individual suspected of having or determined to have cancer), (ii) extracting nucleic acid molecules (e.g., a mixture of tumor or cancer nucleic acid molecules and non-tumor or non-cancer nucleic acid molecules) from the sample, (iii) ligating one or more adapters to the nucleic acid molecules extracted from the sample (e.g., one or more amplification primers, flow cell adaptor sequences, substrate adapter sequences, sample index sequences, or unique molecular identifier (UMI) sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules (e.g., capture nucleic acid molecules) that each comprise a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the individual (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in a graphical user interface of an online or web-based healthcare portal. In some instances, the report is transmitted via a computer network or peer-to-peer connection.

In some embodiments of any of the methods provided herein, the methods may comprise one or more of the steps of: (a) providing a plurality of nucleic acid molecules obtained from a sample from an individual (e.g., an individual suspected of having or determined to have cancer), wherein the plurality of nucleic acid molecules comprises nucleic acid molecules corresponding to a RET fusion nucleic acid molecule of the disclosure; (b) ligating one or more adapters onto one or more nucleic acid molecules from the plurality of nucleic acid molecules; (c) amplifying the one or more ligated nucleic acid molecules from the plurality of nucleic acid molecules; (d) capturing amplified nucleic acid molecules from the amplified nucleic acid molecules; (c) sequencing, by a sequencer, the captured nucleic acid molecules to obtain a plurality of sequence reads that represent the captured nucleic acid molecules, wherein one or more of the plurality of sequence reads correspond to the RET fusion nucleic acid molecule; (f) analyzing the plurality of sequence reads; and (g) based on the analysis, detecting the presence or absence of the RET fusion nucleic acid molecule in the sample. In some embodiments, the methods further comprise receiving, at one or more processors, sequence read data for the plurality of sequence reads. In some embodiments, the analyzing the plurality of sequence reads comprises identifying, using the one or more processors, the presence or absence of sequence reads corresponding to the RET fusion nucleic acid molecule. In some embodiments, the amplified nucleic acid molecules are captured by hybridization with one or more bait molecules.

In some embodiments of any of the methods provided herein, the methods may comprise one or more of the steps of: (a) providing a sample from an individual (e.g., an individual suspected of having or determined to have cancer), wherein the sample comprises a plurality of nucleic acid molecules; (b) preparing a nucleic acid sequencing library from the plurality of nucleic acid molecules in the sample; (c) amplifying said library; (d) selectively enriching for one or more nucleic acid molecules comprising nucleotide sequences corresponding to a RET fusion nucleic acid molecule of the disclosure in said library to produce an enriched sample; (e) sequencing the enriched sample, thereby producing a plurality of sequence reads; (f) analyzing the plurality of sequence reads for the presence of the RET fusion nucleic acid molecule; (g) detecting, based on the analyzing step, the presence or absence of the RET fusion nucleic acid molecule in the sample from the individual.

In some embodiments of any of the methods provided herein, the plurality of nucleic acid molecules comprises a mixture of cancer nucleic acid molecules and non-cancer nucleic acid molecules. In some embodiments, the cancer nucleic acid molecules are derived from a tumor portion of a heterogeneous tissue biopsy sample, and the non-cancer nucleic acid molecules are derived from a normal portion of the heterogeneous tissue biopsy sample. In some embodiments, the sample comprises a liquid biopsy sample, and the cancer nucleic acid molecules are derived from a circulating tumor DNA (ctDNA) fraction of the liquid biopsy sample; and the non-cancer nucleic acid molecules are derived from a non-tumor fraction of the liquid biopsy sample or a cell-free DNA (cfDNA) fraction of the liquid biopsy sample.

In some embodiments of any of the methods, the one or more adapters comprise amplification primers, flow cell adaptor sequences, substrate adapter sequences, sample index sequences, or unique molecular identifier (UMI) sequences. In some embodiments, the one or more adapters comprise one or more sample index sequences. As is known in the art, sample indexes allow the sequencing of multiple samples on the same instrument flow cell or chip (i.e., multiplexing). Sample indexes are typically between about 8 and about 10 bases in length, and comprise a nucleotide sequence specific to a sample that is used to assign sequence reads to the correct sample during data analysis. In some embodiments, the one or more adapters comprise one or more unique molecule identifiers (UMIs). As is known in the art, UMIs comprise short nucleotide sequences that include a unique barcode that is incorporated into each molecule in a given sample library. UMIs are useful for identifying PCR duplicates created during library amplification steps, and/or for reducing the rate of false-positive variant calls and increasing variant detection, since variant alleles present in the original sample (true variants) can be distinguished from errors introduced during library preparation, target enrichment, or sequencing.

In some embodiments of any of the methods provided herein, the methods comprise selectively enriching for one or more nucleic acids in a sample comprising nucleotide sequences corresponding to a RET fusion nucleic acid molecule of the disclosure. In some embodiments, selectively enriching comprises: (a) combining one or more bait molecules with a sequencing library, thereby hybridizing the one or more bait molecules to one or more nucleic acid molecules comprising nucleotide sequences corresponding to a RET fusion nucleic acid molecule and producing nucleic acid hybrids; and (b) isolating the nucleic acid hybrids to produce an enriched sample. In other embodiments, the selectively enriching comprises: (a) combining one or more bait molecules with a sample, thereby hybridizing the one or more bait molecules to one or more nucleic acids in the sample comprising nucleotide sequences corresponding to the RET fusion nucleic acid molecule and producing nucleic acid hybrids; and (b) isolating the nucleic acid hybrids to produce an enriched sample. In other embodiments, the selectively enriching comprises amplifying one or more nucleic acids comprising nucleotide sequences corresponding to a RET fusion nucleic acid molecule using a polymerase chain reaction (PCR) to produce an enriched sample. In other embodiments, nucleic acid molecules comprising nucleotide sequences corresponding to a RET fusion nucleic acid molecule are captured from amplified nucleic acid molecules by hybridization to one or more bait molecules. In some embodiments, the methods further comprise sequencing the enriched sample or the captured nucleic acid molecules. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencing is performed using a sequencer, optionally a next generation sequencer.

In some embodiments of any of the methods provided herein, the methods further comprise analyzing sequence data (e.g., obtained from sequencing as described above), for the presence or absence of one or more alterations (e.g., a base substitution, a short insertion/deletion (indel), a copy number alteration, or a genomic rearrangement) in one or more genes. In some embodiments, the one or more genes comprise one or more known/suspected oncogenes and/or tumor suppressors, one or more cancer-related genes, or any combination thereof. Alternatively or additionally, in some embodiments, the one or more genes comprise one or more of ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKNIA, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNFIA, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MSTIR, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2RIA, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSCIL1, WT1, XPO1, XRCC2, ZNF217, ZNF703, or any combination thereof. Alternatively or additionally, in some embodiments, the one or more genes comprise one or more of ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1B, IL-6, IL-6R, JAK1, JAK2, JAK3, KIT, KRAS, MEK, MET, MSI-H, mTOR, PARP, PD-1, PDGFR, PDGFRa, PDGFRB, PD-L1, PI3K8, PIGF, PTCH, RAF, RANKL, RET, ROS1, SLAMF7, VEGF, VEGFA, VEGFB, or any combination thereof. Alternatively or additionally, in some embodiments, the one or more genes comprise one or more of CPEB3, OPTN, GRM7, AGBL4, PPP2R5A, OXR1, LRMDA, MGEA5, ADCY1, SAMD4A, HSD17B7P2, ACPP, ZNF248, MKX, MYH14, BMS1, GOLGB1, NTRK2, ABI3BP, ARHGAP19, GP2, SH2D3A, VSTM4, RBMS3, LINC00379, ZNF721, ZSWIM6, SGIP1, DCC, CCNY, WDFY4, EEA1, ELMO1, RAD1, NAALADL2, CCBE1, CEP135, RAI14, ADAMTS14, SORBS1, CSGALNACT2, PCM1, KIAA1217, MPRIP, TFG, SPECC1L, REEP3, RRBP1, ETV6, TAF3, RASGEF1A, TNIP2, SATB1, ALOX5, ANKRD26, CLIP1, DLG5, ERC1, FRMD4A, KIAA1468, NCOA4, PARD3, PRKAR1A, PRKG1, RUFY2, SNRNP70, SQSTM1, TNIP1, TRIM27, TRIM33, CCDC6, TRIM24, FGFR1OP, GAS2, HOOK1, LMNA, MLPH, MYH9, PCDH15, PIBF1, SLC12A2, or ZNF485, or any gene listed in Tables 1-2, and any combination thereof. In some embodiments, the presence or absence of the one or more gene alterations is detected using any suitable method known in the art, e.g., as described in Frampton et al., (2013) Nat Biotechnol. 31:1023-1031. In some embodiments, base substitution alterations are detected using Bayesian methodology, which allows detection of novel somatic mutations at low mutant allele frequency (MAF) and increased sensitivity for mutations at hotspot sites through the incorporation of tissue-specific prior expectations. See, e.g., Kim et al., Cancer Discov (2011) 1:44-53 and Frampton et al., (2013) Nat Biotechnol. 31:1023-1031. In some embodiments, insertion/deletion (indel) alterations are detected using any suitable method, such as de novo local assembly, e.g., using the de Bruijn approach, see, e.g., Compeau et al., Nat Biotechnol (2011) 29: 987-991 and Frampton et al., (2013) Nat Biotechnol. 31:1023-1031. In some embodiments, gene fusion and genomic rearrangement alterations are detected using any suitable method, such as by analyzing chimeric read pairs (read pairs for which reads map to separate chromosomes, or at a distance of over 10 Mbp), see, e.g., Frampton et al., (2013) Nat Biotechnol, 31:1023-1031. In some embodiments, rearrangements are annotated for predicted function (e.g., creation of fusion gene or tumor suppressor inactivation).

In some embodiments of any of the methods provided herein, the methods further comprise generating a molecular profile for the individual or the sample, based, at least in part, on detecting the presence or absence of a RET fusion nucleic acid molecule of the disclosure. In some embodiments, the molecular profile for the individual or sample further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the molecular profile further comprises results from a nucleic acid sequencing-based test. In some instances, a molecular profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some embodiments of any of the methods provided herein, the methods further comprise selecting a treatment, administering a treatment, or applying a treatment to the individual based on the generated molecular profile, wherein the treatment comprises an anti-cancer therapy, e.g., as described herein, e.g., a RET-targeted therapy. In some embodiments of any of the methods provided herein, the methods further comprise generating a report indicating the presence or absence of a RET fusion nucleic acid molecule of the disclosure, in the sample. In some embodiments of any of the methods provided herein, the methods further comprise generating, by one or more processors, a report indicating the presence or absence of a RET fusion nucleic acid molecule of the disclosure in the sample. In some embodiments, the report comprises the generated molecular profile. In some embodiments, the methods further comprise providing or transmitting the report, e.g., as described below. In some embodiments, the report is transmitted via a computer network or a peer-to-peer connection. In some instances, all or a portion of the report may be displayed in a graphical user interface of an online or web-based healthcare portal.

In some embodiments of any of the methods provided herein, the methods for determining the presence or absence of a RET fusion nucleic acid molecule, may be implemented as part of a genomic profiling process that comprises identification of the presence of variant sequences at one or more gene loci (e.g., one or more genes as listed above) in a sample derived from an individual as part of detecting, monitoring, predicting a risk factor, or selecting a treatment for a particular disease, e.g., cancer. In some instances, the variant panel selected for genomic profiling may comprise the detection of variant sequences at a selected set of gene loci (e.g., one or more genes as listed above). In some instances, the variant panel selected for genomic profiling may comprise detection of variant sequences at a number of gene loci (e.g., one or more genes) through comprehensive genomic profiling (CGP), a next-generation sequencing (NGS) approach used to assess hundreds of genes (including relevant cancer biomarkers) in a single assay. Inclusion of the disclosed methods for determining the presence or absence of a RET fusion nucleic acid molecule as part of a genomic profiling process can improve the validity of, e.g., disease detection calls by, for example, independently confirming the presence of the RET fusion nucleic acid molecule in a given patient sample.

The disclosed methods may be used with any of a variety of samples, e.g., as described in further detail below. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, nucleic acid molecules extracted from a sample may comprise a mixture of tumor or cancer nucleic acid molecules and non-tumor or non-cancer nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor or cancer nucleic acid molecules may be derived from a circulating tumor DNA (ctDNA) fraction of the liquid biopsy sample while the non-tumor or non-cancer nucleic acid molecules may be derived from a non-tumor or non-cancer, cell-free DNA (cfDNA) fraction of the liquid biopsy sample. In some embodiments of any of the methods provided herein, the method further comprises determining the circulating tumor DNA (ctDNA) fraction of a liquid biopsy sample.

### (ii) Detection of RET Fusion Polypeptides

Also provided herein are methods of detecting a RET fusion polypeptide of the disclosure (e.g., any of the RET fusion polypeptides described above and/or in the Examples herein), or a fragment thereof, in a sample.

A RET fusion polypeptide provided herein, or a fragment thereof, may be detected or measured, e.g., in a sample obtained from an individual, using any method known in the art, such as using antibodies (e.g., an antibody described herein), mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography).

In some embodiments, a RET fusion polypeptide provided herein, or a fragment thereof, can be distinguished from a reference polypeptide, e.g., a non-mutant or wild type protein or polypeptide, with an antibody or antibody fragment that reacts differentially with a mutant protein or polypeptide (e.g., a RET fusion polypeptide provided herein or a fragment thereof) as compared to a reference protein or polypeptide. In some embodiments, a RET fusion polypeptide of the disclosure, or a fragment thereof, can be distinguished from a reference polypeptide, e.g., a non-mutant or wild type protein or polypeptide, by reaction with a detection reagent, e.g., a substrate, e.g., a substrate for catalytic activity, e.g., phosphorylation.

In some aspects, methods of detection of a RET fusion polypeptide of the disclosure, or a fragment thereof, are provided, comprising contacting a sample, e.g., a sample described herein, comprising a RET fusion polypeptide described herein, with a detection reagent provided herein (e.g., an antibody of the disclosure), and determining if the RET fusion polypeptide is present in the sample.

(iii) Detection Reagents

In some aspects, provided herein are reagents for detecting a RET fusion nucleic acid molecule of the disclosure, or a fragment thereof (e.g., any of the RET fusion nucleic acid molecules described above and/or in the Examples herein), e.g., according to the methods of detection provided herein. In some embodiments, a detection reagent provided herein comprises a nucleic acid molecule, e.g., a DNA, RNA, or mixed DNA/RNA molecule, comprising a nucleotide sequence that is complementary to a nucleotide sequence on a target nucleic acid molecule, e.g., a nucleic acid molecule that is or comprises a RET fusion nucleic acid molecule described herein or a fragment or portion thereof.

In other aspects, provided herein are reagents for detecting a RET fusion polypeptide of the disclosure (e.g., any of the RET fusion polypeptides described above and/or in the Examples herein), or a fragment thereof, e.g., according to the methods of detection provided herein. In some embodiments, a detection reagent provided herein comprises an antibody or antibody fragment that specifically binds to a RET fusion polypeptide of the disclosure, or to a fragment thereof.

### Baits

In some embodiments, nucleic acids corresponding to a gene involved in a RET fusion nucleic acid molecule described herein, e.g., a RET gene, and/or a corresponding gene fusion partner as described herein (e.g., in Tables 1-10, and/or in the Examples herein), are captured (e.g., from amplified nucleic acids) by hybridization with a bait molecule. Provided herein are bait molecules suitable for the detection of a RET fusion nucleic acid molecule of the disclosure (e.g., any of the RET fusion nucleic acid molecules described above and/or in the Examples herein).

In some embodiments, a bait molecule comprises a capture nucleic acid molecule configured to hybridize to a target nucleic acid molecule comprising a RET fusion nucleic acid molecule of the disclosure, or a fragment or portion thereof. In some embodiments, the capture nucleic acid molecule is configured to hybridize to the RET fusion nucleic acid molecule of the target nucleic acid molecule.

In some embodiments, the capture nucleic acid molecule is configured to hybridize to a fragment of a RET fusion nucleic acid molecule of the disclosure. In some embodiments, the fragment comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the fragment comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length. In some embodiments, the fragment comprises a breakpoint or fusion junction of a RET fusion nucleic acid molecule of the disclosure. In some embodiments, the fragment comprises any of at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more, nucleotides in length. In some embodiments, the fragment comprises between about 5 and about 100 nucleotides, between about 10 and about 50 nucleotides, or between about 10 and about 20 nucleotides, including any specific value within each of the recited ranges. In some embodiments, the fragment comprises any of at least about 1, 2, 3, 4, 5, 6, 7, 8, 9, or 10 nucleotides on either side of the breakpoint or fusion junction, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more, nucleotides on either side of the breakpoint or fusion junction.

In some embodiments, the capture nucleic acid molecule comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the capture nucleic acid molecule comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length.

In some embodiments, the capture nucleic acid molecule is configured to hybridize to a breakpoint of a RET fusion nucleic acid molecule of the disclosure, and may further hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the breakpoint.

In some embodiments, the capture nucleic acid molecule is configured to hybridize to a nucleotide sequence in an intron or an exon of a RET gene, or in a breakpoint joining the introns or exons of a RET gene (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides), and/or to an intron or exon of another gene (e.g., a corresponding gene fusion partner as described herein, e.g., in Tables 1-10, and/or in the Examples herein).

In some embodiments, the capture nucleic acid molecule is a DNA, RNA, or a DNA/RNA molecule. In some embodiments, the capture nucleic acid molecule comprises any of between about 50 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the capture nucleic acid molecule comprises any of between about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides. In some embodiments, the capture nucleic acid molecule comprises about 150 nucleotides. In some embodiments, the capture nucleic acid molecule is about 150 nucleotides. In some embodiments, the capture nucleic acid molecule comprises about 170 nucleotides. In some embodiments, the capture nucleic acid molecule is about 170 nucleotides.

In some embodiments, a bait provided herein comprises a DNA, RNA, or a DNA/RNA molecule. In some embodiments, a bait provided herein includes a label, a tag or detection reagent. In some embodiments, the label, tag or detection reagent is a radiolabel, a fluorescent label, an enzymatic label, a sequence tag, biotin, or another ligand. In some embodiments, a bait provided herein includes a detection reagent such as a fluorescent marker. In some embodiments, a bait provided herein includes (e.g., is conjugated to) an affinity tag or reagent, e.g., that allows capture and isolation of a hybrid formed by a bait and a nucleic acid molecule hybridized to the bait. In some embodiments, the affinity tag or reagent is an antibody, an antibody fragment, biotin, or any other suitable affinity tag or reagent known in the art. In some embodiments, a bait is suitable for solution phase hybridization.

Baits can be produced and used according to methods known in the art, e.g., as described in WO2012092426A1 and/or or in Frampton et al (2013) Nat Biotechnol, 31:1023-1031, incorporated herein by reference. For example, biotinylated baits (e.g., RNA baits) can be produced by obtaining a pool of synthetic long oligonucleotides, originally synthesized on a microarray, and amplifying the oligonucleotides to produce the bait sequences. In some embodiments, the baits are produced by adding an RNA polymerase promoter sequence at one end of the bait sequences, and synthesizing RNA sequences using RNA polymerase. In one embodiment, libraries of synthetic oligodeoxynucleotides can be obtained from commercial suppliers, such as Agilent Technologies, Inc., and amplified using known nucleic acid amplification methods.

In some embodiments, a bait provided herein is between about 100 nucleotides and about 300 nucleotides. In some embodiments, a bait provided herein is between about 130 nucleotides and about 230 nucleotides. In some embodiments, a bait provided herein is between about 150 nucleotides and about 200 nucleotides. In some embodiments, a bait provided herein comprises a target-specific bait sequence (e.g., a capture nucleic acid molecule described herein) and universal tails on each end. In some embodiments, the target-specific sequence, e.g., a capture nucleic acid molecule described herein, is between about 40 nucleotides and about 300 nucleotides. In some embodiments, the target-specific sequence, e.g., a capture nucleic acid molecule described herein, is between about 100 nucleotides and about 200 nucleotides. In some embodiments, the target-specific sequence, e.g., a capture nucleic acid molecule described herein, is between about 120 nucleotides and about 170 nucleotides. In some embodiments, the target-specific sequence, e.g., a capture nucleic acid molecule described herein, is about 150 nucleotides or about 170 nucleotides. In some embodiments, a bait provided herein comprises an oligonucleotide comprising about 200 nucleotides, of which about 150 nucleotides or about 170 nucleotides are target-specific (e.g., a capture nucleic acid molecule described herein), and the other 50 nucleotides or 30 nucleotides (e.g., 25 or 15 nucleotides on each end of the bait) are universal arbitrary tails, e.g., suitable for PCR amplification.

In some embodiments, a bait provided herein hybridizes to a nucleotide sequence corresponding to an intron or an exon of one gene of a RET fusion nucleic acid molecule described herein (e.g., a RET gene), in an intron or an exon of the other gene of a RET fusion nucleic acid molecule described herein (e.g., a corresponding gene fusion partner as described herein, e.g., in any of Tables 1-10, and/or in the Examples herein), and/or a breakpoint joining the introns and/or exons.

The baits described herein can be used for selection of exons and short target sequences.

In some embodiments, a bait of the disclosure distinguishes a nucleic acid molecule, e.g., a genomic or transcribed nucleic acid molecule, e.g., a cDNA or RNA, having a breakpoint of a RET fusion nucleic acid molecule described herein from a reference nucleotide sequence, e.g., a nucleotide sequence not having the breakpoint.

In some embodiments, the bait hybridizes to a breakpoint of a RET fusion nucleic acid molecule described herein and a sequence on either side of the breakpoint (e.g., any of 1, 2, 3, 4, 5, 6, 7, 8, 9, or 10 nucleotides on either side of the breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the breakpoint).

### Probes

Also provided herein are probes, e.g., nucleic acid molecules, suitable for the detection of a RET fusion nucleic acid molecule of the disclosure (e.g., any of the RET fusion nucleic acid molecules described above and/or in the Examples herein). In some embodiments, a probe provided herein comprises a nucleic acid sequence configured to hybridize to a target nucleic acid molecule that is or comprises a RET fusion nucleic acid molecule of the disclosure, or a fragment or portion thereof. In some embodiments, the probe comprises a nucleic acid sequence configured to hybridize to the RET fusion nucleic acid molecule of the disclosure, or the fragment or portion thereof, of the target nucleic acid molecule. In some embodiments, the probe comprises a nucleic acid sequence configured to hybridize to a fragment or portion of the RET fusion nucleic acid molecule of the target nucleic acid molecule. In some embodiments, the fragment or portion comprises between about 5 and about 25 nucleotides, between about 5 and about 300 nucleotides, between about 100 and about 300 nucleotides, between about 130 and about 230 nucleotides, or between about 150 and about 200 nucleotides.

In some embodiments, the probe comprises a nucleotide sequence configured to hybridize to a breakpoint of a RET fusion nucleic acid molecule of the disclosure, and may be further configured to hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the breakpoint.

In some embodiments, the probe comprises a nucleotide sequence configured to hybridize to a nucleotide sequence in an intron or an exon of a gene involved in a RET fusion nucleic acid molecule described herein, e.g., a RET gene, or in a breakpoint joining the introns or exons of the gene (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides), to an intron or exon of another gene (e.g., a corresponding gene fusion partner as described herein, e.g., in any of Tables 1-10, and/or in the Examples herein).

In some embodiments, the probe comprises a nucleic acid molecule which is a DNA, RNA, or a DNA/RNA molecule. In some embodiments, the probe comprises a nucleic acid molecule comprising any of between about 10 and about 20 nucleotides, between about 12 and about 20 nucleotides, between about 10 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the probe comprises a nucleic acid molecule comprising any of 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides. In some embodiments, the probe comprises a nucleic acid molecule comprising any of between about 40 nucleotides and about 50 nucleotides, about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the probe comprises a nucleic acid molecule comprising between about 12 and about 20 nucleotides.

In some embodiments, a probe provided herein comprises a DNA, RNA, or a DNA/RNA molecule. In some embodiments, a probe provided herein includes a label or a tag. In some embodiments, the label or tag is a radiolabel (e.g., a radioisotope), a fluorescent label (e.g., a fluorescent compound), an enzymatic label, an enzyme co-factor, a sequence tag, biotin, or another ligand. In some embodiments, a probe provided herein includes a detection reagent such as a fluorescent marker. In some embodiments, a probe provided herein includes (e.g., is conjugated to) an affinity tag, e.g., that allows capture and isolation of a hybrid formed by a probe and a nucleic acid molecule hybridized to the probe. In some embodiments, the affinity tag is an antibody, an antibody fragment, biotin, or any other suitable affinity tag or reagent known in the art. In some embodiments, a probe is suitable for solution phase hybridization.

In some embodiments, probes provided herein may be used according to the methods of detection of RET fusion nucleic acid molecules provided herein. For example, a probe provided herein may be used for detecting a RET fusion nucleic acid molecule of the disclosure in a sample. e.g., a sample obtained from an individual. In some embodiments, the probe may be used for identifying cells or tissues that express a RET fusion nucleic acid molecule of the disclosure, e.g., by measuring levels of the RET fusion nucleic acid molecule. In some embodiments, the probe may be used for detecting levels of a RET fusion nucleic acid molecule of the disclosure, e.g., mRNA levels, in a sample of cells from an individual.

In some embodiments, a probe provided herein specifically hybridizes to a nucleic acid molecule comprising a rearrangement (e.g., a deletion, inversion, insertion, duplication, or other rearrangement) resulting in a RET fusion nucleic acid molecule of the disclosure.

In some embodiments, a probe of the disclosure distinguishes a nucleic acid, e.g., a genomic or transcribed nucleic acid, e.g., a cDNA or RNA, having a breakpoint of a RET fusion nucleic acid molecule of the disclosure, from a reference nucleotide sequence, e.g., a nucleotide sequence not having the breakpoint.

Also provided herein are isolated pairs of allele-specific probes, wherein, for example, the first probe of the pair specifically hybridizes to a RET fusion nucleic acid molecule of the disclosure, and the second probe of the pair specifically hybridizes to a corresponding wild type sequence. Probe pairs can be designed and produced for any of the RET fusion nucleic acid molecules described herein and are useful in detecting a somatic mutation in a sample. In some embodiments, a first probe of a pair specifically hybridizes to a mutation (e.g., the breakpoint of an alteration, rearrangement, inversion, duplication, deletion, insertion or translocation resulting in a RET fusion nucleic acid molecule described herein), and a second probe of a pair specifically hybridizes to a sequence upstream or downstream of the mutation.

In some embodiments, one or more probes provided herein are suitable for use in in situ hybridization methods, e.g., as described above, such as FISH.

Chromosomal probes, e.g., for use in the FISH methods described herein, are typically about 50 to about 105 nucleotides in length. Longer probes typically comprise smaller fragments of about 100 to about 500 nucleotides. Probes that hybridize with centromeric DNA and locus-specific DNA are available commercially, for example, from Vysis, Inc. (Downers Grove, III.), Molecular Probes, Inc. (Eugene, Oreg.) or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, chromosome (e.g., human chromosome) along with the normal chromosome complement of the host, and chromosomes purified by flow cytometry or microdissection. The region of interest can be isolated through cloning, or by site-specific amplification via the polymerase chain reaction (PCR). Probes of the disclosure may also hybridize to RNA molecules, e.g., mRNA, such as an RNA that is or comprises a RET fusion nucleic acid molecule of the disclosure.

In some embodiments, probes, such as probes for use in the FISH methods described herein, are used for determining whether a cytogenetic abnormality is present in one or more cells, e.g., in a region of a chromosome or an RNA bound by one or more probes provided herein. The cytogenetic abnormality may be a cytogenetic abnormality that results in a RET fusion nucleic acid molecule of the disclosure. Examples of such cytogenetic abnormalities include, without limitation, deletions (e.g., deletions of entire chromosomes or deletions of fragments of one or more chromosomes), duplications (e.g., of entire chromosomes, or of regions smaller than an entire chromosome), translocations (e.g., non-reciprocal translocations, balanced translocations, reciprocal translocations), intra-chromosomal inversions, point mutations, deletions, gene copy number changes, germ-line mutations, and gene expression level changes.

In some embodiments, probes, such as probes for use in the FISH methods described herein, are labeled such that a chromosomal region or a region on an RNA to which the probes hybridize can be detected. Probes typically are directly labeled with a fluorophore, allowing the probe to be visualized without a secondary detection molecule. Probes can also be labeled by nick translation, random primer labeling or PCR labeling. Labeling may be accomplished using fluorescent (direct)- or haptene (indirect)-labeled nucleotides. Representative, non-limiting examples of labels include: AMCA-6-dUTP, CascadeBlue-4-dUTP, Fluorescein-12-dUTP, Rhodamine-6-dUTP, TexasRed-6-dUTP, Cy3-6-dUTP, Cy5-dUTP, Biotin (BIO)-11-dUTP, Digoxygenin (DIG)-11-dUTP and Dinitrophenyl (DNP)-11-dUTP. Probes can also be indirectly labeled with biotin or digoxygenin, or labeled with radioactive isotopes such as ³²P and ³H, and secondary detection molecules may be used, or further processing may be performed, to visualize the probes. For example, a probe labeled with biotin can be detected by avidin conjugated to a detectable marker, e.g., avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase. Probes can also be prepared such that a fluorescent or other label is added after hybridization of the probe to its target to detect that the probe hybridized to the target. For example, probes can be used that have antigenic molecules incorporated into the nucleotide sequence. After hybridization, these antigenic molecules are detected, for example, using specific antibodies reactive with the antigenic molecules. Such antibodies can, for example, themselves incorporate a fluorochrome, or can be detected using a second antibody with a bound fluorochrome. For fluorescent probes, e.g., used in FISH techniques, fluorescence can be viewed with a fluorescence microscope equipped with an appropriate filter for each fluorophore, or by using dual or triple band-pass filter sets to observe multiple fluorophores. Alternatively, techniques such as flow cytometry can be used to examine the hybridization pattern of the chromosomal probes.

In some embodiments, the probe hybridizes to a breakpoint of a RET fusion nucleic acid molecule of the disclosure, and a sequence on either side of the breakpoint (e.g., any of 1, 2, 3, 4, 5, 6, 7, 8, 9, or 10 nucleotides on either side of the breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the breakpoint).

### Oligonucleotides

In some aspects, provided herein are oligonucleotides, e.g., useful as primers. In some embodiments, an oligonucleotide, e.g., a primer, provided herein comprises a nucleotide sequence configured to hybridize to a target nucleic acid molecule that is or comprises a RET fusion nucleic acid molecule of the disclosure (e.g., any of the RET fusion nucleic acid molecules described above and/or in the Examples herein), or a fragment or portion thereof. In some embodiments, the oligonucleotide comprises a nucleotide sequence configured to hybridize to the RET fusion nucleic acid molecule of the target nucleic acid molecule. In some embodiments, the oligonucleotide comprises a nucleotide sequence configured to hybridize to a fragment or portion of the RET fusion nucleic acid molecule of the target nucleic acid molecule.

In some embodiments, the oligonucleotide, e.g., the primer, comprises a nucleotide sequence configured to hybridize to a breakpoint of a RET fusion nucleic acid molecule of the disclosure, and may be further configured to hybridize to between about 10 and about 12, about 12 and about 15, about 15 and about 17, about 17 and about 20, about 20 and about 25, or about 25 and about 30, or more nucleotides flanking either side of the breakpoint.

In some embodiments, the oligonucleotide, e.g., the primer, comprises a nucleotide sequence configured to hybridize to a nucleotide sequence in an intron or an exon of a gene involved in a RET fusion nucleic acid mole of the disclosure (e.g., a RET gene), to a breakpoint of a fusion nucleic acid molecule described herein, and/or to an intron or exon of another gene (e.g., a corresponding gene fusion partner as described herein, e.g., in any of Tables 1-10, and/or in the Examples herein).

In some embodiments, the oligonucleotide comprises a nucleotide sequence corresponding to a RET fusion nucleic acid molecule of the disclosure. In some embodiments, the oligonucleotide comprises a nucleotide sequence corresponding to a fragment or a portion of the RET fusion nucleic acid molecule. In some embodiments, the fragment or portion comprises between about 10 and about 30 nucleotides, between about 12 and about 20 nucleotides, or between about 12 and about 17 nucleotides. In some embodiments, the oligonucleotide comprises a nucleotide sequence complementary to a RET fusion nucleic acid molecule provided herein. In some embodiments, the oligonucleotide comprises a nucleotide sequence complementary to a fragment or a portion of the RET fusion nucleic acid molecule provided herein. In some embodiments, the fragment or portion comprises between about 10 and about 30 nucleotides, between about 12 and about 20 nucleotides, or between about 12 and about 17 nucleotides.

In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence, e.g., under high stringency conditions. In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence under conditions that allow a polymerization reaction (e.g., PCR) to occur.

In some embodiments, an oligonucleotide, e.g., a primer, provided herein may be useful for initiating DNA synthesis via PCR (polymerase chain reaction) or a sequencing method. In some embodiments, the oligonucleotide may be used to amplify a nucleic acid molecule that is or comprises a RET fusion nucleic acid molecule of the disclosure, or a fragment thereof, e.g., using PCR. In some embodiments, the oligonucleotide may be used to sequence a nucleic acid molecule that is or comprises a RET fusion nucleic acid molecule provided herein, or a fragment thereof. In some embodiments, the oligonucleotide may be used to amplify a nucleic acid molecule comprising a breakpoint of a RET fusion nucleic acid molecule described herein, e.g., using PCR. In some embodiments, the oligonucleotide may be used to sequence a nucleic acid molecule comprising a breakpoint of a RET fusion nucleic acid molecule described herein.

In some embodiments, pairs of oligonucleotides, e.g., pairs of primers, are provided herein, which are configured to hybridize to a nucleic acid molecule that is or comprises a RET fusion nucleic acid molecule of the disclosure, or a fragment thereof. In some embodiments, a pair of oligonucleotides of the disclosure may be used for directing amplification of the RET fusion nucleic acid molecule or fragment thereof, e.g., using a PCR reaction. In some embodiments, pairs of oligonucleotides, e.g., pairs of primers, are provided herein, which are configured to hybridize to a nucleic acid molecule comprising a breakpoint of a RET fusion nucleic acid molecule described herein, e.g., for use in directing amplification of the corresponding fusion nucleic acid molecule or fragment thereof, e.g., using a PCR reaction.

In some embodiments, an oligonucleotide, e.g., a primer, provided herein is a single stranded nucleic acid molecule, e.g., for use in sequencing or amplification methods. In some embodiments, an oligonucleotide provided herein is a double stranded nucleic acid molecule. In some embodiments, a double stranded oligonucleotide is treated, e.g., denatured, to separate its two strands prior to use, e.g., in sequencing or amplification methods. Oligonucleotides provided herein comprise a nucleotide sequence of sufficient length to hybridize to their target, e.g., a RET fusion nucleic acid molecule of the disclosure, or a fragment thereof, and to prime the synthesis of extension products, e.g., during PCR or sequencing.

In some embodiments, an oligonucleotide, e.g., a primer, provided herein comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more deoxyribonucleotides or ribonucleotides. In some embodiments, an oligonucleotide provided herein comprises at least about 8 deoxyribonucleotides or ribonucleotides. In some embodiments, an oligonucleotide provided herein comprises at least about 10 deoxyribonucleotides or ribonucleotides. In some embodiments, an oligonucleotide provided herein comprises at least about 12 deoxyribonucleotides or ribonucleotides. In some embodiments, an oligonucleotide provided herein comprises at least about 15 deoxyribonucleotides or ribonucleotides. In some embodiments, an oligonucleotide provided herein comprises at least about 20 deoxyribonucleotides or ribonucleotides. In some embodiments, an oligonucleotide provided herein comprises at least about 30 deoxyribonucleotides or ribonucleotides. In some embodiments, an oligonucleotide provided herein comprises between about 10 and about 30 deoxyribonucleotides or ribonucleotides. In some embodiments, an oligonucleotide provided herein comprises between about 10 and about 25 deoxyribonucleotides or ribonucleotides. In some embodiments, an oligonucleotide provided herein comprises between about 10 and about 20 deoxyribonucleotides or ribonucleotides. In some embodiments, an oligonucleotide provided herein comprises between about 10 and about 15 deoxyribonucleotides or ribonucleotides. In some embodiments, an oligonucleotide provided herein comprises between about 12 and about 20 deoxyribonucleotides or ribonucleotides. In some embodiments, an oligonucleotide provided herein comprises between about 17 and about 20 deoxyribonucleotides or ribonucleotides. In some embodiments, the length and nucleotide sequence of an oligonucleotide provided herein is determined according to methods known in the art, e.g., based on factors such as the specific application (e.g., PCR, sequencing library preparation, sequencing), reaction conditions (e.g., buffers, temperature), and the nucleotide composition of the nucleotide sequence of the oligonucleotide or of its target complementary sequence.

In some embodiments, an oligonucleotide, e.g., a primer, of the disclosure distinguishes a nucleic acid, e.g., a genomic or transcribed nucleic acid, e.g., a cDNA or RNA, having a breakpoint of a RET fusion nucleic acid molecule described herein, from a reference nucleotide sequence, e.g., a nucleotide sequence not having the breakpoint.

In one aspect, provided herein is a primer or primer set for amplifying a nucleic acid molecule comprising a cytogenetic abnormality such as an alteration, rearrangement, chromosomal inversion, deletion, translocation, duplication, or other rearrangement resulting in a RET fusion nucleic acid molecule of the disclosure. In another aspect, provided herein is a primer or primer set for amplifying a nucleic acid molecule comprising an alteration, rearrangement, chromosomal inversion, insertion, deletion, translocation, duplication or other rearrangement resulting in a RET fusion nucleic acid molecule of the disclosure. In certain aspects, provided herein are allele-specific oligonucleotides, e.g., primers, wherein a first oligonucleotide of a pair specifically hybridizes to a mutation (e.g., a breakpoint of a RET fusion nucleic acid molecule described herein), and a second oligonucleotide of a pair specifically hybridizes to a sequence upstream or downstream of the mutation. In certain aspects, provided herein are pairs of oligonucleotides, e.g., primers, wherein a first oligonucleotide of a pair specifically hybridizes to a sequence upstream of a mutation (e.g., a breakpoint of a RET fusion nucleic acid molecule described herein), and a second oligonucleotide of the pair specifically hybridizes to a sequence downstream of the mutation.

In some embodiments, the oligonucleotide, e.g., the primer, hybridizes to a breakpoint of a RET fusion nucleic acid molecule described herein, and a sequence on either side of the breakpoint (e.g., any of 1, 2, 3, 4, 5, 6, 7, 8, 9, or 10 nucleotides on either side of the breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the breakpoint).

### Antibodies

Provided herein are antibodies or antibody fragments that specifically bind to a RET fusion polypeptide of the disclosure (e.g., e.g., any of the RET fusion polypeptides described above and/or in the Examples herein), or a fragment thereof.

The antibody may be of any suitable type of antibody, including, but not limited to, a monoclonal antibody, a polyclonal antibody, a multi-specific antibody (e.g., a bispecific antibody), or an antibody fragment, so long as the antibody or antibody fragment exhibits a specific antigen binding activity, e.g., binding to a RET fusion polypeptide of the disclosure, or a fragment thereof.

In some embodiments, a RET fusion polypeptide of the disclosure, or a fragment thereof, is used as an immunogen to generate one or more antibodies of the disclosure, e.g., using standard techniques for polyclonal and monoclonal antibody preparation. In some embodiments, a RET fusion polypeptide provided herein, is used to provide antigenic peptide fragments (e.g., comprising any of at least about 8, at least about 10, at least about 15, at least about 20, at least about 30 or more amino acids) for use as immunogens to generate one or more antibodies of the disclosure, e.g., using standard techniques for polyclonal and monoclonal antibody preparation. As is known in the art, an antibody of the disclosure may be prepared by immunizing a suitable (i.e., immunocompetent) subject such as a rabbit, goat, mouse, or other mammal or vertebrate. An appropriate immunogenic preparation can contain, for example, recombinantly-expressed or chemically-synthesized polypeptides, e.g., a RET fusion polypeptide of the disclosure, or a fragment thereof. The preparation can further include an adjuvant, such as Freund's complete or incomplete adjuvant, or a similar immunostimulatory agent.

In some embodiments, an antibody provided herein is a polyclonal antibody. Methods of producing polyclonal antibodies are known in the art. In some embodiments, an antibody provided herein is a monoclonal antibody, wherein a population of the antibody molecules contain only one species of an antigen binding site capable of immunoreacting or binding with a particular epitope, e.g., an epitope on a RET fusion polypeptide provided herein. Methods of preparation of monoclonal antibodies are known in the art, e.g., using standard hybridoma techniques originally described by Kohler and Milstein (1975) Nature 256:495-497, human B cell hybridoma techniques (see Kozbor et al., 1983, Immunol. Today 4:72), EBV-hybridoma techniques (see Cole et al., pp. 77-96 In Monoclonal Antibodies and Cancer Therapy, Alan R. Liss, Inc., 1985), or trioma techniques. The technology for producing hybridomas is well known (see generally Current Protocols in Immunology, Coligan et al, ed., John Wiley & Sons, New York, 1994). A monoclonal antibody of the disclosure may also be identified and isolated by screening a recombinant combinatorial immunoglobulin library (e.g., an antibody phage display library) with the polypeptide of interest, e.g., a RET fusion polypeptide provided herein or a fragment thereof. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display libraries can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; and Griffiths et al. (1993) EMBO J. 12:725-734. In some embodiments, monoclonal antibodies of the disclosure are recombinant antibodies, such as chimeric and humanized monoclonal antibodies, comprising both human and non-human portions. Such chimeric and/or humanized monoclonal antibodies can be produced by recombinant DNA techniques known in the art, for example, using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559; Morrison (1985) Science 229:1202-1207; Oi et al. (1986) Bio/Techniques 4:214; U.S. Pat. No. 5,225,539; Jones et al. (1986) Nature 321:552-525; Verhocyan et al. (1988) Science 239:1534; and Beidler et al. (1988) J. Immunol. 141:4053-4060. In some embodiments, a monoclonal antibody of the disclosure is a human monoclonal antibody. In some embodiments, human monoclonal antibodies are prepared using methods known in the art, e.g., using transgenic mice which are incapable of expressing endogenous immunoglobulin heavy and light chains genes, but which can express human heavy and light chain genes. For an overview of this technology for producing human antibodies, see Lonberg and Huszar (1995) Int. Rev. Immunol. 13:65-93. For a detailed discussion of this technology for producing human antibodies and human monoclonal antibodies, and protocols for producing such antibodies, see, e.g., U.S. Pat. Nos. 5,625,126; 5,633,425; 5,569,825; 5,661,016; and 5,545,806.

In some embodiments, the antibody or antibody fragment of the disclosure is an isolated antibody or antibody fragment, which has been separated from a component of its natural environment or a cell culture used to produce the antibody or antibody fragment. In some embodiments, an antibody of the disclosure is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods.

In some embodiments, an antibody of the disclosure can be used to isolate a RET fusion polypeptide provided herein, or a fragment thereof, by standard techniques, such as affinity chromatography or immunoprecipitation. In some embodiments, an antibody of the disclosure can be used to detect a RET fusion polypeptide provided herein, or a fragment thereof, e.g., in a tissue sample, cellular lysate, or cell supernatant, in order to evaluate the level and/or pattern of expression of the fusion polypeptide. Detection can be facilitated by coupling the antibody to a detectable substance. Thus, in some embodiments, an antibody of the disclosure is coupled to a detectable substance, such as enzymes, prosthetic groups, fluorescent materials, luminescent materials, bioluminescent materials, and radioactive materials. Non-limiting examples of suitable enzymes include, e.g., horseradish peroxidase, alkaline phosphatase, β-galactosidase, or acetylcholinesterase; examples of suitable prosthetic group complexes include, e.g., streptavidin/biotin and avidin/biotin; examples of suitable fluorescent materials include, e.g., umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, e.g., luciferase, luciferin, and aequorin; and examples of suitable radioactive materials include, e.g., ¹²⁵I, ¹³¹I, ³⁵S or ³H.

An antibody or antibody fragment of the disclosure may also be used diagnostically, e.g., to detect and/or monitor protein levels (e.g., protein levels of a RET fusion polypeptide provided herein) in tissues or body fluids (e.g., in a tumor cell-containing tissue or body fluid), e.g., according to the methods provided herein.

In certain embodiments, an antibody provided herein has a dissociation constant (Kd) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). Methods of measuring antibody affinity (e.g., Kd) are known in the art, and include, without limitation, a radiolabeled antigen binding assay (RIA) and a BIACORE® surface plasmon resonance assay. In some embodiments, antibody affinity (e.g., Kd) is determined using the Fab version of an antibody of the disclosure and its antigen (e.g., a RET fusion polypeptide provided herein). In some embodiments, a RIA is performed with the Fab version of an antibody of the disclosure and its antigen (e.g., a RET fusion polypeptide provided herein).

In certain embodiments, an antibody provided herein is an antibody fragment. Antibody fragments include, but are not limited to, Fab, Fab′, Fab′-SH, F(ab)₂, Fv, and single-chain antibody molecule (e.g., scFv) fragments, and other fragments described herein or known in the art.

In certain embodiments, an antibody provided herein is a diabody. Diabodies are antibody fragments with two antigen-binding sites that may be bivalent or bispecific. In certain embodiments, an antibody provided herein is a triabody or a tetrabody.

In certain embodiments, an antibody provided herein is a single-domain antibody. Single-domain antibodies are antibody fragments comprising all or a portion of the heavy chain variable domain or all or a portion of the light chain variable domain of an antibody. In certain embodiments, a single-domain antibody is a human single-domain antibody.

Antibody fragments can be made by various techniques, including but not limited to proteolytic digestion of an intact antibody, as well as production by recombinant host cells (e.g., E. coli or phage), as known in the art and as described herein.

In certain embodiments, an antibody provided herein is a chimeric antibody. In one example, a chimeric antibody comprises a non-human variable region (e.g., a variable region derived from a mouse, rat, hamster, rabbit, or non-human primate, such as a monkey), and a human constant region. In a further example, a chimeric antibody is a “class switched” antibody, in which the class or subclass of the antibody has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof), are derived from a non-human antibody, and framework regions (FRs) (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity. Humanized antibodies and methods of making them are known in the art. Human framework regions that may be used for humanization include but are not limited to: framework regions selected using the “best-fit” method; framework regions derived from the consensus sequence of human antibodies of a particular subgroup of light or heavy chain variable regions; human mature (somatically mutated) framework regions or human germline framework regions; and framework regions derived from screening FR libraries.

In certain embodiments, an antibody provided herein is a human antibody. Human antibodies can be produced using various techniques known in the art. For example, human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or are present extrachromosomally or integrated randomly into the animal's chromosomes. In such transgenic animals, e.g., mice, the endogenous immunoglobulin loci have generally been inactivated. Human variable regions from intact antibodies generated by such animals may be further modified, e.g., by combining with a different human constant region. Human antibodies can also be made by hybridoma-based methods known in the art, e.g., using known human myeloma and mouse-human heteromyeloma cell lines for the production of human monoclonal antibodies. Human antibodies may also be generated by isolating Fv clone variable domain sequences selected from human-derived phage display libraries. Such variable domain sequences may then be combined with a desired human constant domain. Techniques for selecting human antibodies from antibody libraries are known in the art and described herein.

Antibodies of the disclosure may be isolated by screening combinatorial libraries for antibodies with the desired activity or activities. For example, a variety of methods are known in the art for generating phage display libraries and screening such libraries for antibodies possessing the desired binding characteristics. In certain phage display methods, repertoires of VH and VL genes are separately cloned by polymerase chain reaction (PCR) and recombined randomly in phage libraries, which can then be screened for antigen-binding phage. Phage typically display antibody fragments, either as single-chain Fv (scFv) fragments or as Fab fragments. Libraries from immunized sources provide high-affinity antibodies to the immunogen without the requirement of constructing hybridomas. Alternatively, a naive antibody repertoire can be cloned (e.g., from human) to provide a single source of antibodies to a wide range of non-self and also self antigens without any immunization. Naive libraries can also be made synthetically by cloning un-rearranged V-gene segments from stem cells, and using PCR primers containing random sequences to amplify the highly variable CDR3 regions and to accomplish rearrangement in vitro. Antibodies or antibody fragments isolated from human antibody libraries are considered human antibodies or human antibody fragments herein.

In certain embodiments, an antibody provided herein is a multispecific antibody, e.g., a bispecific antibody. Multispecific antibodies are monoclonal antibodies that have binding specificities for at least two different sites or at least two different antigens. For example, one of the binding specificities can be to a RET fusion polypeptide of the disclosure, and the other can be to any other antigen. Multispecific antibodies can be prepared as full length antibodies or as antibody fragments. Techniques for making multispecific antibodies are known in the art and include, but are not limited to, recombinant co-expression of two immunoglobulin heavy chain-light chain pairs having different specificities, and “knob-in-hole” engineering. Multispecific antibodies may also be made by engineering electrostatic steering effects (e.g., by introducing mutations in the constant region) for making heterodimeric Fes; cross-linking two or more antibodies or fragments; using leucine zippers to produce bispecific antibodies: using “diabody” technology for making bispecific antibody fragments; using single-chain Fv (scFv) dimers; and preparing trispecific antibodies. Engineered antibodies with three or more functional antigen binding sites, including “Octopus antibodies.” are also included in the disclosure. Antibodies or antibody fragments of the disclosure also include “Dual Acting FAbs” or “DAF.” e.g., comprising an antigen binding site that binds to a RET fusion polypeptide of the disclosure as well as another, different antigen.

In certain embodiments, amino acid sequence variants of the antibodies provided herein are contemplated. For example, it may be desirable to improve the binding affinity and/or other biological properties of the antibody. Amino acid sequence variants of an antibody of the disclosure may be prepared by introducing appropriate modifications into the nucleotide sequence encoding the antibody, or by peptide synthesis. Such modifications include, for example, deletions, and/or insertions, and/or substitutions of residues within the amino acid sequences of the antibody. Any combination of deletions, insertions, and substitutions can be made to arrive at the final antibody, provided that the final antibody possesses the desired characteristics, e.g., antigen-binding.

In certain embodiments, antibody variants having one or more amino acid substitutions are provided. Sites of interest for substitutional mutagenesis include the HVRs and FRs. Amino acid substitutions may be introduced into an antibody of interest, and the products may be screened for a desired activity, e.g., retained/improved antigen binding, decreased immunogenicity, or improved or reduced antibody-dependent cell-mediated cytotoxicity (ADCC) and/or complement-dependent cytotoxicity (CDC).

In certain embodiments, an antibody of the present disclosure is altered to increase or to decrease the extent to which the antibody is glycosylated. Addition or deletion of glycosylation sites to an antibody may be conveniently accomplished by altering the amino acid sequence of the antibody, such that one or more glycosylation sites is created or removed. Antibody variants having bisected oligosaccharides are further provided, e.g., in which a biantennary oligosaccharide attached to the Fc region of the antibody is bisected by GlcNAc. In some embodiments, antibody variants of the disclosure may have increased fucosylation. In some embodiments, antibody variants of the disclosure may have reduced fucosylation. In some embodiments, antibody variants of the disclosure may have improved ADCC function. In some embodiments, antibody variants of the disclosure may have decreased ADCC function. Antibody variants with at least one galactose residue in the oligosaccharide attached to the Fc region are also provided. Such antibody variants may have improved CDC function. In some embodiments, antibody variants of the disclosure may have increased CDC function. In some embodiments, antibody variants of the disclosure may have decreased CDC function.

In certain embodiments, one or more amino acid modifications may be introduced into the Fc region of an antibody of the present disclosure, thereby generating an Fe region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the present disclosure contemplates an antibody variant that possesses some but not all effector functions, which make it a desirable candidate for applications in which the half-life of the antibody in vivo is important, yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks Fc-gamma-R binding (hence likely lacking ADCC activity), but retains FcRn binding ability. The primary cells that mediate ADCC, e.g., NK cells, express Fc-gamma-RIII only, whereas monocytes express Fc-gamma-RI, Fc-gamma-RII and Fc-gamma-RIII. Antibodies with reduced effector function include those with substitution of one or more of Fc region residues 238, 265, 269, 270, 297, 327 and 329. Such Fc mutants include Fc mutants with substitutions at two or more of amino acid positions 265, 269, 270, 297 and 327, including the so-called “DANA” Fc mutant with substitutions of residues 265 and 297 to alanine. Antibody variants with improved or diminished binding to FeRs are also included in the disclosure. In certain embodiments, an antibody variant comprises an Fc region with one or more amino acid substitutions that improve ADCC, e.g., substitutions at positions 298, 333, and/or 334 of the Fc region. In some embodiments, numbering of Fe region residues is according to EU numbering of residues. In some embodiments, alterations are made in the Fc region that result in altered (i.e., either improved or diminished) C1q binding and/or CDC. In some embodiments, antibodies of the disclosure include antibodies with increased half-lives and improved binding to the neonatal Fc receptor (FcRn), e.g., comprising one or more substitutions that improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434. See, also, Duncan & Winter, Nature 322:738-40 (1988); U.S. Pat. Nos. 5,648,260; 5,624,821; and WO 94/29351 for other examples of Fc region variants.

In certain embodiments, an antibody provided herein is a cysteine-engineered antibody, e.g., “thioMAb,” in which one or more residues of the antibody are substituted with cysteine residues. In some embodiments, the substituted residues occur at accessible sites of the antibody. By substituting those residues with cysteine, reactive thiol groups are thereby positioned at accessible sites of the antibody, and may be used to conjugate the antibody to other moieties, such as drug moieties or linker-drug moieties, e.g., to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine-engineered antibodies may be generated using any suitable method known in the art.

In some embodiments, an antibody or antibody fragment provided herein comprises a label or a tag. In some embodiments, the label or tag is a radiolabel, a fluorescent label, an enzymatic label, a sequence tag, biotin, or other ligands. Examples of labels or tags include, but are not limited to, 6xHis-tag, biotin-tag, Glutathione-S-transferase (GST)-tag, green fluorescent protein (GFP)-tag, c-myc-tag, FLAG-tag, Thioredoxin-tag, Glu-tag, Nus-tag, V5-tag, calmodulin-binding protein (CBP)-tag, Maltose binding protein (MBP)-tag, Chitin-tag, alkaline phosphatase (AP)-tag, HRP-tag, Biotin Caboxyl Carrier Protein (BCCP)-tag, Calmodulin-tag, S-tag, Strep-tag, haemoglutinin (HA)-tag, digoxigenin (DIG)-tag, DsRed, RFP, Luciferase, Short Tetracysteine Tags, Halo-tag, and Nus-tag. In some embodiments, the label or tag comprises a detection agent, such as a fluorescent molecule or an affinity reagent or tag.

In some embodiments, an antibody or antibody fragment provided herein is conjugated to a drug molecule, e.g., an anti-cancer agent described herein, or a cytotoxic agent such as mertansine or monomethyl auristatin E (MMAE).

In certain embodiments, an antibody or antibody fragment provided herein may be further modified to contain additional nonproteinaceous moieties. Such moieties may be suitable for derivatization of the antibody, e.g., including but not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyamino acids (either homopolymers or random copolymers), and dextran or poly (n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, polyethylene glycol propionaldehyde, and mixtures thereof. The polymers may be of any molecular weight, and may be branched or unbranched. The number of polymers attached to the antibody may vary, and if more than one polymer is attached, the polymers can be the same or different molecules. In general, the number and/or type of polymers used for derivatization can be determined based on considerations including, but not limited to, the particular properties or functions of the antibody to be improved, or whether the antibody derivative will be used in a therapy under defined conditions. In some embodiments, provided herein are antibodies conjugated to carbon nanotubes, e.g., for use in methods to selectively heat the antibody using radiation to a temperature at which cells proximal to the antibody are killed.

### (iv) Samples

A variety of materials can be the source of, or serve as, samples for use in any of the methods of the disclosure, such as the methods for detection of a RET fusion nucleic acid molecule or a RET fusion polypeptide of the disclosure, or fragments thereof.

For example, the sample can be, or be derived from: solid tissue such as from a fresh, frozen and/or preserved organ, tissue sample, biopsy (e.g., tumor, tissue or liquid biopsy), resection, smear, or aspirate; scrapings; bone marrow or bone marrow specimens; a bone marrow aspirate; blood or any blood constituents; blood cells; bodily fluids such as cerebrospinal fluid, amniotic fluid, urine, saliva, sputum, peritoneal fluid or interstitial fluid; pleural fluid; ascites; tissue or fine needle biopsy samples; surgical specimens; cell-containing body fluids; free-floating nucleic acids; feces; lymph; gynecological fluids; skin swabs; vaginal swabs; oral swabs; nasal swabs; washings or lavages such as ductal lavages or bronchoalveolar lavages; cells from any time in gestation or development of an individual; cells from a cancer or tumor; other body fluids, secretions, and/or excretions, and/or cells therefrom. In some embodiments, a sample is or comprises cells obtained from an individual. In some embodiments, the sample is or is derived from blood or blood constituents, e.g., obtained from a liquid biopsy. In some embodiments, the sample is or is derived from a tumor sample. In some embodiments, the sample is or comprises biological tissue or fluid. In some embodiments, the sample can contain compounds that are not naturally intermixed with the source of the sample in nature, such as preservatives, anticoagulants, buffers, fixatives, nutrients, antibiotics or the like. In some embodiments, the sample is preserved as a frozen sample or as a formaldehyde- or paraformaldehyde-fixed paraffin-embedded (FFPE) tissue preparation. In some embodiments, the sample comprises circulating tumor cells (CTCs).

In one embodiment, the sample comprises one or more cells associated with a tumor, e.g., tumor cells or tumor-infiltrating lymphocytes (TIL). In one embodiment, the sample includes one or more premalignant or malignant cells. In one embodiment, the sample is acquired from a hematologic malignancy (or pre-malignancy), e.g., a hematologic malignancy (or pre-malignancy) described herein. In one embodiment, the sample is acquired from a cancer, such as a cancer described herein. In some embodiments, the sample is acquired from a solid tumor, a soft tissue tumor or a metastatic lesion. In other embodiments, the sample includes tissue or cells from a surgical margin. In one embodiment, the sample is or is acquired from a liquid biopsy of blood, plasma, cerebrospinal fluid, sputum, stool, urine, or saliva. In some embodiments, the sample includes cell-free DNA (cfDNA) and/or circulating tumor DNA (ctDNA), e.g., from a biopsy of blood, plasma, cerebrospinal fluid, sputum, stool, urine, or saliva. In another embodiment, the sample includes one or more circulating tumor cells (CTCs) (e.g., a CTC acquired from a blood sample). In one embodiment, the sample is a cell not associated with a tumor or cancer, e.g., a non-tumor or non-cancer cell or a peripheral blood lymphocyte.

In some embodiments, a sample is a primary sample obtained directly from a source of interest by any appropriate means. For example, in some embodiments, a primary biological sample is obtained by a method chosen from biopsy (e.g., fine needle aspiration or tissue biopsy), surgery, or collection of body fluid (e.g., blood, lymph, or feces). In some embodiments, as will be clear from context, the term “sample” refers to a preparation that is obtained by processing (e.g., by removing one or more components of and/or by adding one or more agents to) a primary sample. Such a processed sample may comprise, for example, nucleic acids (e.g., for use in any of the methods for detection of RET fusion nucleic acid molecules provided herein) or proteins (e.g., for use in any of the methods for detection of RET fusion polypeptides provided herein) extracted from a sample or obtained by subjecting a primary sample to techniques such as amplification methods, reverse transcription of mRNA, or isolation and/or purification of certain components such as nucleic acids and/or proteins.

In some embodiments, the sample comprises nucleic acids, e.g., genomic DNA, cDNA, or mRNA. In some embodiments, the sample comprises cell-free DNA (cfDNA). In some embodiments, the sample comprises cell-free RNA (cfRNA). In some embodiments, the sample comprises circulating tumor DNA (ctDNA). In certain embodiments, the nucleic acids are purified or isolated (e.g., removed from their natural state). In some embodiments, the sample comprises tumor or cancer nucleic acids, such as nucleic acids from a tumor or cancer sample, e.g., genomic DNA, RNA, or cDNA derived from RNA, or from a liquid biopsy, e.g., ctDNA from blood, plasma, cerebrospinal fluid, sputum, stool, urine, or saliva. In certain embodiments, a tumor or cancer nucleic acid sample, or a ctDNA sample, is purified or isolated (e.g., it is removed from its natural state).

In some embodiments, the sample comprises tumor or cancer proteins or polypeptides, such as proteins or polypeptides from a tumor or a cancer sample, or from a liquid biopsy, e.g., from blood, plasma, cerebrospinal fluid, sputum, stool, urine, or saliva. In certain embodiments, the proteins or polypeptides are purified or isolated (e.g., removed from their natural state).

In some embodiments, the sample is obtained from an individual having a cancer, such as a cancer described herein. In some embodiments, the sample comprises a RET fusion nucleic acid molecule or a RET fusion polypeptide of the disclosure.

In some embodiments, the sample is a control sample or a reference sample, e.g., not containing a RET fusion nucleic acid molecule or a RET fusion polypeptide described herein. In certain embodiments, the reference sample is purified or isolated (e.g., it is removed from its natural state). In certain embodiments, the reference or control sample comprises a wild type or a non-mutated nucleic acid molecule or polypeptide counterpart to any of the RET fusion nucleic acid molecules or RET fusion polypeptides described herein. In other embodiments, the reference sample is from a non-tumor or cancer sample, e.g., a blood control, a normal adjacent tumor (NAT), or any other non-cancerous sample from the same or a different individual.

In some embodiments, a RET fusion nucleic acid molecule of the disclosure is detected in a sample comprising genomic or subgenomic DNA fragments, or RNA (e.g., mRNA), isolated from a sample, e.g., a tumor or cancer sample, a normal adjacent tissue (NAT) sample, a tissue sample, or a blood, plasma, cerebrospinal fluid, sputum, stool, urine, or saliva sample obtained from an individual. In some embodiments, the sample comprises cDNA derived from an mRNA sample or from a sample comprising mRNA. In some embodiments, a RET fusion nucleic acid molecule of the disclosure is detected in a sample comprising cell-free DNA (cfDNA), cell-free RNA, and/or circulating tumor DNA (ctDNA). In some embodiments, a RET fusion nucleic acid molecule of the disclosure is detected in a sample comprising cell-free DNA (cfDNA) and/or circulating tumor DNA (ctDNA). In some embodiments, a RET fusion nucleic acid molecule of the disclosure is detected in a sample comprising circulating tumor DNA (ctDNA).

### C. Anti-Cancer Therapies

Certain aspects of the present disclosure relate to anti-cancer therapies, as well as methods for identifying an individual having a cancer who may benefit from a treatment comprising an anti-cancer therapy; selecting a treatment for an individual having a cancer; identifying one or more treatment options for an individual having a cancer; predicting survival of an individual having a cancer; treating or delaying progression of cancer; monitoring, evaluating or screening an individual having a cancer; detecting the presence or absence of a cancer in an individual; monitoring progression or recurrence of a cancer in an individual; or identifying a candidate treatment for a cancer in an individual in need thereof. The present disclosure also provides uses for anti-cancer therapies (e.g., in methods of treating or delaying progression of cancer in an individual, or in methods for manufacturing a medicament for treating or delaying progression of cancer). In some instances, the methods of the disclosure can include administering an anti-cancer therapy or applying an anti-cancer therapy to an individual based on a generated molecular and/or sequencing mutation profile. An anti-cancer therapy can refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

In some embodiments, an anti-cancer therapy of the disclosure is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer comprising a RET fusion nucleic acid molecule or polypeptide, a treatment for cancer being tested in a clinical trial, a targeted therapy, a treatment being tested in a clinical trial for cancer comprising a RET fusion nucleic acid molecule or polypeptide, or any combination thereof, e.g., a described in further detail below. In some embodiments, the anti-cancer therapy is a RET-targeted therapy. In some embodiments, the anti-cancer therapy is a kinase inhibitor, such as a kinase inhibitor described herein or known in the art. In some embodiments, the kinase inhibitor is a multi-kinase inhibitor or a RET-specific inhibitor known in the art or described herein. In some embodiments, the nucleic acid inhibits the expression of a RET fusion nucleic acid molecule or polypeptide of the disclosure.

In some embodiments, an anti-cancer therapy of the disclosure is a RET-targeted therapy, e.g., as described herein or known in the art. In some embodiments, the RET-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for RET-positive or RET-rearranged cancer, a RET-targeted therapy being tested in a clinical trial, a treatment for RET-positive or RET-rearranged cancer being tested in a clinical trial, or any combination thereof. In some embodiments, the RET-targeted therapy is a kinase inhibitor known in the art or described herein. In some embodiments, the RET-targeted therapy is a tyrosine kinase inhibitor known in the art or described herein. In some embodiments, the RET-targeted therapy is a multi-kinase inhibitor or a RET-specific inhibitor known in the art or described herein. In some embodiments, the kinase inhibitor inhibits the kinase activity of a RET polypeptide. In some embodiments, the RET-targeted therapy comprises one or more of pralsctinib, selpercatinib, lenvatinib, sorafenib, sunitinib, vandetanib, NVP-AST487, regorafenib, dovitinib, motesanib, cabozantinib, lapatinib, lestaurtinib, linifanib, semaxinib, ponatinib, fostamatinib, quizartinib, imatinib, vatalanib, ENMD-2076, JNJ-26483327, DCC-2157, Zetelctinib, TPX0046, TAS0953, RXDX-105, LOXO-260, BOS172738, Alectinib, APS03118, LOX-18228, or SYHA1815. In some embodiments, the nucleic acid inhibits the expression of a RET fusion nucleic acid molecule or polypeptide of the disclosure.

In some embodiments, the RET-targeted therapy is pralsctinib. Pralsctinib has demonstrated clinical benefit for patients with various tumor types harboring RET rearrangements (Gainor et al., Registrational dataset from the phase I/II ARROW trial of pralsetinib (BLU-667) in patients (pts) with advanced RET fusion+non-small cell lung cancer (NSCLC). Journal of Clinical Oncology 2020 38: 15_suppl, 9515-9515; Subbiah et al., Clinical activity of the RET inhibitor pralsetinib (BLU-667) in patients with RET fusion-positive solid tumors. Journal of Clinical Oncology 2021 39: 3_suppl, 467-467). The Phase 1/2 ARROW study of pralsetinib reported ORRs of 73% (12% CR) and 61% (5% CR) for patients with RET-fusion-positive non-small cell lung cancer in the first-line and prior platinum settings, respectively (Gainor et al., Registrational dataset from the phase I/II ARROW trial of pralsctinib (BLU-667) in patients (pts) with advanced RET fusion+non-small cell lung cancer (NSCLC). Journal of Clinical Oncology 2020 38: 15_suppl, 9515-9515). In this same study, patients with RET-fusion-positive thyroid cancer exhibited an ORR of 91% (10/11; 10 PR), whereas patients with various other RET-fusion-positive solid malignancies achieved an ORR of 50% (6/12; 6 PR); these responses were seen in patients with pancreatic adenocarcinoma (3/3 PR), cholangiocarcinoma (2/2 PR), and unknown primary neuroendocrine cancer (1/1 PR) (Subbiah et al., Clinical activity of the RET inhibitor pralsetinib (BLU-667) in patients with RET fusion-positive solid tumors. Journal of Clinical Oncology 2021 39: 3_suppl, 467-467). See, also Belli, C., et al., Progresses Toward Precision Medicine in RET-altered Solid Tumors. Clin Cancer Res, 2020. 26 (23): p. 6102-6111; Kim. J., et al., FDA Approval Summary: Pralsetinib for the Treatment of Lung and Thyroid Cancers With RET Gene Mutations or Fusions. Clin Cancer Res, 2021. 27 (20): p. 5452-5456; Gainor, J. F., et al., Pralsctinib for RET fusion-positive non-small-cell lung cancer (ARROW): a multi-cohort, open-label, phase 1/2 study. Lancet Oncol, 2021. 22 (7): p. 959-969; Subbiah, V., et al., Pralsetinib for patients with advanced or metastatic RET-altered thyroid cancer (ARROW): a multi-cohort, open-label, registrational, phase 1/2 study. Lancet Diabetes Endocrinol, 2021. 9 (8): p. 491-501; and Subbiah, V., et al., Pan-cancer efficacy of pralsctinib in patients with RET fusion-positive solid tumors from the phase 1/2 ARROW trial. Nat Med, 2022. 28 (8): p. 1640-1645.

In some embodiments, the RET-targeted therapy is selpercatinib. Selpercatinib has demonstrated activity for patients with various tumor types harboring RET rearrangements (Drilon et al. (2020). The New England journal of medicine, 383 (9), 813-824; Wirth et al. (2020). The New England journal of medicine, 383 (9), 825-835; Gerdemann et al., First experience of LOXO-292 in the management of pediatric patients with RET-altered cancers. Journal of Clinical Oncology 2019 37: 15_suppl, 10045-10045; Durham et al. (2019). Nature medicine, 25 (12), 1839-1842). The Phase 1/2 LIBRETTO-001 study reported ORRs of 85% (29/34) and 68% (71/105) for patients with RET-fusion-positive NSCLC in the first line and previously treated settings, respectively (Drilon et al., PL02.08 Registrational Results of LIBRETTO-001: A Phase 1/2 Trial of LOXO-292 in Patients with RET Fusion-Positive Lung Cancers, Journal of Thoracic Oncology, Volume 14, Issue 10, S6-S7). In the same study, patients with RET-fusion-positive thyroid cancer exhibited an ORR of 62% (16/26) (Wirth et al., LBA93-Registrational results of LOXO-292 in patients with RET-altered thyroid cancers, Annals of Oncology, Volume 30, Supplement 5, 2019, p. v933). Among patients in LIBRETTO-001 with RET-fusion-positive tumor types other than lung or thyroid, the ORR was 47% (15/32) with responses reported in colon, pancreatic, carcinoid, small intestine, salivary, xanthogranuloma, breast, ovarian, and sarcoma (Subbiah et al: Efficacy and safety of selpercatinib in RET fusion-positive cancers other than lung or thyroid cancers. AACR Annual Meeting 2021. Abstract CT011.). See, also Belli, C., et al., Progresses Toward Precision Medicine in RET-altered Solid Tumors. Clin Cancer Res, 2020. 26 (23): p. 6102-6111; Bradford, D., et al., FDA Approval Summary: Selpercatinib for the Treatment of Lung and Thyroid Cancers with RET Gene Mutations or Fusions. Clin Cancer Res, 2021. 27 (8): p. 2130-2135; Drilon, A., et al., Efficacy of Selpercatinib in RET Fusion-Positive Non-Small-Cell Lung Cancer. N Engl J Med, 2020. 383 (9): p. 813-824; and Wirth, L. J., et al., Efficacy of Selpercatinib in RET-Altered Thyroid Cancers. N Engl J Med. 2020. 383 (9): p. 825-835.

In some embodiments, the RET-targeted therapy is cabozantinib. A Phase 2 study of cabozantinib in RET-rearranged lung adenocarcinoma reported an ORR of 28% (7 PRs, n=25), a median PFS of 5.5 months, and an OS of 9.9 months (Drilon et al. (2016). The Lancet. Oncology, 17 (12), 1653-1660; Drilon et al. (2013). Cancer discovery, 3 (6), 630-635). In a retrospective analysis of patients with RET-rearranged non-small cell lung cancer treated with various RET inhibitors, of the 19 evaluable patients treated with cabozantinib, 1 CR and 6 PRs were reported, reaching an ORR of 37% (Gautschi et al. (2017). Journal of clinical oncology 35 (13), 1403-1410). In other studies and case reports, varying degrees of clinical benefit have been reported for patients with RET-rearranged lung cancer treated with cabozantinib (Nokihara et al. (2019). Clinical lung cancer, 20 (3), e317-e328; Mukhopadhyay et al. (2014). Journal of thoracic oncology, 9 (11), 1714-1719; Zhang et al., 2021; WCLC Abstract P87.03; Xu et al., 2020; ESMO Abstract 415P; Fei et al., 2018; IASLC Abstract P081; Xing et al. (2020). Translational Cancer Research, 9 (10), 6455-6463; Overbeck and Schildhaus, 2017; IASLC Abstract P3.02c-017; Wang e al. (2019). Medicine, 98 (3), e14120; Sarfaty et al. (2017). Clinical lung cancer, 18 (4), e223-e232; Zheng et al. (2020). Journal of thoracic oncology, 15 (8), e132-c133; Nokihara et al., 2013; JSMO Abstract 02-026; Chae et al. (2019). JCO precision oncology, 3, PO.18.00295; Abbar et al. (2019). Journal of thoracic oncology, 14 (11), e250-e251; Drilon et al. (2015). Clinical cancer research, 21 (16), 3631-3639; Michels et al. (2016). Journal of thoracic oncology, 11 (1), 122-127).

In some embodiments, the RET-targeted therapy is lenvatinib. A Phase 2 study of lenvatinib for patients with RET-rearranged lung adenocarcinoma reported an ORR of 16% (4 PRs, n=25) with a median PFS of 7.3 months and median OS not reached (Hida et al. (2019). Lung cancer (Amsterdam, Netherlands), 138, 124-130). In the Global Multicenter RET Registry (GLORY), 1 PR and 1 PD were reported among the 2 patients with advanced RET-rearranged lung cancer treated with lenvatinib (Gautschi et al. (2017). Journal of clinical oncology, 35 (13), 1403-1410). Another retrospective study identified 1 patient with EGFR-mutated RET-rearranged non-small cell lung cancer (NSCLC) who benefited from lenvatinib (Yao et al., RET fusion in first/third-generation EGFR-TKIs resistance in advanced non-small cell lung cancer. Journal of Clinical Oncology 2019 37: 15_suppl, e20634-e20634). A retrospective study of patients with fusion-positive thyroid carcinoma identified 3 patients treated with lenvatinib in the first-line setting; 1 patient with RET-rearranged anaplastic thyroid carcinoma experienced a best overall response of SD before progressing after 20 months of treatment, whereas the other 2 patients, 1 with poorly differentiated thyroid carcinoma and the other with papillary thyroid carcinoma, did not respond (Chu et al. (2020). Modern pathology, 33 (12), 2458-2472). In a case report, a patient with suspected RET-rearranged undifferentiated thyroid cancer with brain metastases experienced a PR following treatment with lenvatinib (Li et al., 2019; ATA Abstract 291).

In some embodiments, the RET-targeted therapy is sorafenib. Two studies reported 1 PR each in patients with RET-rearranged papillary thyroid carcinoma (Chen et al. (2011). Thyroid, 21 (2), 119-124) or chronic myelomonocytic leukemia (Ballerini et al. (2012). Leukemia, 26 (11), 2384-2389). Of 5 patients with RET-rearranged NSCLC treated with sorafenib, 3 achieved SD while 2 had PD; all patients had been previously treated with at least one line of chemotherapy (Gautschi et al. (2017). Journal of clinical oncology, 35 (13), 1403-1410; Horiike et al. (2016). Lung cancer (Amsterdam, Netherlands), 93, 43-46).

In some embodiments, the RET-targeted therapy is sunitinib. For patients with RET-rearranged non-small cell lung cancer treated with sunitinib, a retrospective study reported 2 PRs and 3 SDs among 9 evaluable patients (Gautschi et al. (2017). Journal of clinical oncology, 35 (13), 1403-1410). Additional studies of heavily pretreated patients with RET-rearranged lung adenocarcinoma have reported 1 PR (Wu et al. (2015). Journal of thoracic oncology, 10 (9), e95-e96), 1 SD (Lee et al., 2015; AACR Abstract 2416), and 1 patient who showed an initial mixed response followed by PD (Lee et al., 2015; AACR Abstract 2416).

In some embodiments, the RET-targeted therapy is vandetanib. Clinical studies indicate that vandetanib is active against RET-rearranged non-small cell lung cancer (NSCLC). The Global Multicenter RET Registry (GLORY) reported an ORR of 18% (2 PRs, n=11), median PFS (mPFS) of 2.9 months, and median OS (mOS) of 10.2 months for patients with advanced RET-rearranged lung cancer treated with vandetanib (Gautschi et al. (2017). Journal of clinical oncology, 35 (13), 1403-1410). Phase 2 studies of vandetanib for patients with previously treated RET-rearranged NSCLC reported ORRs of 18-47% with mPFS of 4.5-6.5 months and mOS of 11.6-13.5 months (Yoh et al. (2021). Lung cancer (Amsterdam, Netherlands), 155, 40-45; Lee et al. (2017). Annals of oncology, 28 (2), 292-297). A retrospective study of vandetanib for Korean patients with RET-rearranged NSCLC reported an ORR of 16% (3 PRs, n=19) with mPFS of 2.9 months and mOS of 9.3 months (Lee et al. (2020). Japanese journal of clinical oncology, 50 (5), 594-601). A retrospective analysis of the Phase 3 ZODIAC, ZEAL, ZEPHYR, and ZEST studies identified 1 SD and 2 PDs among 3 patients with RET-rearranged NSCLC treated with vandetanib (Platt et al. (2015). BMC cancer, 15, 171). Multiple case reports have also reported varying degrees of clinical benefit for patients with RET-rearranged lung cancer following treatment with vandetanib (Gautschi et al. (2013). Journal of thoracic oncology. 8 (5), e43-e44; Drilon et al. (2018). Journal of thoracic oncology, 13 (10), 1595-1601; Dagogo-Jack et al. (2018). Journal of thoracic oncology, 13 (11), e226-e227; Loh et al. (2019). Internal medicine journal, 49 (12), 1541-1545; Falchook et al. (2016). Journal of clinical oncolog, 34 (15), e141-c144; Varella-Garcia et al., RET rearrangements detected by FISH in “pan-negative” lung adenocarcinoma. Journal of Clinical Oncology 2013 31: 15_suppl, 8024-8024). In a Phase 1 study of vandetanib combined with the mTOR inhibitor everolimus, clinical benefit was observed for 6/6 patients with RET-fusion-positive NSCLC, including 5/6 PRs (Cascone et al., Safety, toxicity and activity of multi-kinase inhibitor vandetanib in combination with everolimus in advanced solid tumors. Journal of Clinical Oncology 2016 34: 15_suppl, 9073-9073). This combination has also demonstrated intracranial activity for a patient with RET-rearranged NSCLC and brain metastases (Subbiah et al. (2015). Lung cancer (Amsterdam, Netherlands), 89 (1), 76-79).

In some embodiments, an anti-cancer therapy of the disclosure (e.g., a RET-targeted therapy) is administered in combination with an additional anti-cancer therapy. In some embodiments, the additional anti-cancer therapy is any anti-cancer therapy known in the art or described herein. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

In some embodiments, an anti-cancer therapy of the disclosure comprises a cyclin-dependent kinase (CDK) inhibitor, e.g., alone or in combination with a RET-targeted therapy. In some embodiments, the CDK inhibitor inhibits CDK4. In some embodiments, the CDK inhibitor inhibits Cyclin D/CDK4. In some embodiments, the CDK inhibitor is (a) a small molecule that inhibits one or more enzymatic activities of CDK4, (b) an antibody that inhibits one or more activities of CDK4 (e.g., by binding to and inhibiting one or more activities of CDK4, binding to and inhibiting expression of CDK4, and/or binding to and inhibiting one or more activities of a cell expressing CDK4, such as by inducing antibody-dependent cellular cytotoxicity, ADCC, or phagocytosis, ADCP), or (c) a nucleic acid that inhibits expression of CDK4 (e.g., an antisense oligonucleotide, miRNA, siRNA, morpholino, CRISPR-based therapeutic, and the like). In some embodiments, the CDK inhibitor inhibits CDK4 and CDK6. In some embodiments, the CDK inhibitor is a small molecule inhibitor of CDK4 (e.g., a competitive or non-competitive inhibitor). Non-limiting examples of CDK inhibitors include palbociclib, ribociclib, and abemaciclib, as well as pharmaceutically acceptable salts thereof.

In some embodiments, an anti-cancer therapy of the disclosure comprises a murine double minute 2 homolog (MDM2) inhibitor, e.g., alone or in combination with a RET-targeted therapy. In some embodiments, the MDM2 inhibitor is (a) a small molecule that inhibits one or more activities of MDM2 (e.g., binding to p53), (b) an antibody that inhibits one or more activities of MDM2 (e.g., by binding to and inhibiting one or more activities of MDM2, binding to and inhibiting expression of MDM2, and/or binding to and inhibiting one or more activities of a cell expressing MDM2, such as by inducing antibody-dependent cellular cytotoxicity, ADCC, or phagocytosis, ADCP), or (c) a nucleic acid that inhibits expression of MDM2 (e.g., an antisense oligonucleotide, miRNA, siRNA, morpholino, CRISPR-based therapeutic, and the like). In some embodiments, the MDM2 inhibitor is a small molecule inhibitor of MDM2 (e.g., a competitive or non-competitive inhibitor). Non-limiting examples of MDM2 inhibitors include nutlin-3a, RG7112, idasanutlin (RG7388), AMG-232, MI-63, MI-291, MI-391, MI-77301 (SAR405838), APG-115, DS-3032b, NVP-CGM097, and HDM-201 (siremadlin), as well as pharmaceutically acceptable salts thereof. In some embodiments, the MDM2 inhibitor inhibits or disrupts interaction between MDM2 and p53.

In some embodiments, an anti-cancer therapy of the disclosure comprises (alone or in combination with a RET-targeted therapy) one or more of an antimetabolite. DNA-damaging agent, or platinum-containing therapeutic (e.g., 5-azacitadine, 5-fluorouracil, acadesine, busulfan, carboplatin, cisplatin, chlorambucil, CPT-11, cytarabine, daunorubicin, decitabine, doxorubicin, etoposide, fludarabine, gemcitabine, idarubicin, radiation, oxaliplatin, temozolomide, topotecan, trabectedin, GSK2830371, or rucaparib); a pro-apoptotic agent (e.g., a BCL2 inhibitor or downregulator, SMAC mimetic, or TRAIL agonist such as ABT-263, ABT-737, oridonin, venetoclax, combination of venetoclax and an anti-CD20 antibody such as obinutuzumab or rituximab, 1396-11, ABT-10, SM-164. D269H/E195R, or rhTRAIL); a tyrosine kinase inhibitor (e.g., as described herein); an inhibitor of RAS, RAF, MEK, or the MAPK pathway (e.g., AZD6244, dabrafenib, LGX818, PD0325901, pimasertib, trametinib, or vemurafenib); an inhibitor of PI3K, mTOR, or Akt (e.g., as described herein); a CDK inhibitor (e.g., as described herein); a PKC inhibitor (e.g., LXS196 or sotrastaurin); an antibody-based therapeutic (e.g., an anti-PD-1 or anti-PDL1 antibody such as atezolizumab, pembrolizumab, nivolumab, or spartalizumab; an anti-CD20 antibody such as obinutuzumab or rituximab; or an anti-DR5 antibody such as drozitumab); a proteasome inhibitor (e.g., bortezomib, carfilzomib, ixazomib, or MG-132); an HDAC inhibitor (e.g., SAHA or VPA); an antibiotic (e.g., actinomycin D); a zinc-containing therapeutic (e.g., zinc or ZMC1); an HSP inhibitor (e.g., geldanamycin); an ATPase inhibitor (e.g., archazolid); a mitotic inhibitor (e.g., paclitaxel or vincristine); metformin; methotrexate; tanshinone IIA; and/or P5091.

In some embodiments, an anti-cancer therapy of the disclosure comprises a tyrosine kinase inhibitor, e.g., alone or in combination with a RET-targeted therapy. In some embodiments, the tyrosine kinase inhibitor is (a) a small molecule that inhibits one or more enzymatic activities of a tyrosine kinase, (b) an antibody that inhibits one or more activities of a tyrosine kinase (e.g., by binding to and inhibiting one or more activities of the tyrosine kinase, binding to and inhibiting expression, such as cell surface expression, of the tyrosine kinase, and/or binding to and inhibiting one or more activities of a cell expressing the tyrosine kinase, such as by inducing antibody-dependent cellular cytotoxicity, ADCC, or phagocytosis, ADCP), or (c) a nucleic acid that inhibits expression of a tyrosine kinase (e.g., an antisense oligonucleotide, miRNA, siRNA, morpholino, CRISPR-based therapeutic, and the like). In some embodiments, the tyrosine kinase inhibitor is a small molecule inhibitor of a tyrosine kinase (e.g., a competitive or non-competitive inhibitor). Non-limiting examples of tyrosine kinase inhibitors include imatinib, crenolanib, linifanib, ninetedanib, axitinib, dasatinib, imetelstat, midostaurin, pazopanib, sorafenib, sunitinb, motesanib, masitinib, vatalanib, cabozanitinib, tivozanib, OSI-930, Ki8751, telatinib, dovitinib, tyrphostin AG 1296, and amuvatinib, as well as pharmaceutically acceptable salts thereof.

In some embodiments, an anti-cancer therapy of the disclosure comprises a mitogen-activated protein kinase (MEK) inhibitor, e.g., alone or in combination with a RET-targeted therapy. In some embodiments, the MEK inhibitor inhibits one or more activities of MEK1 and/or MEK2. In some embodiments, the anti-cancer therapy/MEK inhibitor is (a) a small molecule that inhibits one or more enzymatic activities of MEK, (b) an antibody that inhibits one or more activities of MEK (e.g., by binding to and inhibiting one or more activities of MEK, binding to and inhibiting expression of MEK, and/or binding to and inhibiting one or more activities of a cell expressing MEK, such as by inducing antibody-dependent cellular cytotoxicity. ADCC, or phagocytosis, ADCP), or (c) a nucleic acid that inhibits expression of MEK (e.g., an antisense oligonucleotide, miRNA, siRNA, morpholino, CRISPR-based therapeutic, and the like). In some embodiments, the MEK inhibitor is a small molecule inhibitor of MEK (e.g., a competitive or non-competitive inhibitor). Non-limiting examples of MEK inhibitors include trametinib, cobimetinib, binimetinib, CI-1040, PD0325901, selumetinib, AZD8330, TAK-733, GDC-0623, refametinib, pimasertib, RO4987655, RO5126766, WX-544, and HL-085, as well as pharmaceutically acceptable salts thereof. In some embodiments, the anti-cancer therapy inhibits one or more activities of the Raf/MEK/ERK pathway, including inhibitors of Raf, MEK, and/or ERK.

In some embodiments, an anti-cancer therapy of the disclosure comprises a mammalian target of rapamycin (mTOR) inhibitor, e.g., alone or in combination with a RET-targeted therapy. In some embodiments, the mTOR inhibitor is (a) a small molecule that inhibits one or more enzymatic activities of mTOR. (b) an antibody that inhibits one or more activities of mTOR (e.g., by binding to and inhibiting one or more activities of mTOR, binding to and inhibiting expression of mTOR, and/or binding to and inhibiting one or more activities of a cell expressing mTOR, such as by inducing antibody-dependent cellular cytotoxicity, ADCC, or phagocytosis, ADCP), or (c) a nucleic acid that inhibits expression of mTOR (e.g., an antisense oligonucleotide, miRNA, siRNA, morpholino, CRISPR-based therapeutic, and the like). In some embodiments, the mTOR inhibitor is a small molecule inhibitor of mTOR (e.g., a competitive inhibitor, such as an ATP-competitive inhibitor, or a non-competitive inhibitor, such as a rapamycin analog). Non-limiting examples of mTOR inhibitors include temsirolimus, everolimus, ridaforolimus, dactolisib, GSK2126458, XL765, AZD8055, AZD2014, MLN128, PP242, NVP-BEZ235, LY3023414, PQR309, PKI587, and OSI027, as well as pharmaceutically acceptable salts thereof. In some embodiments, the anti-cancer therapy inhibits one or more activities of the Akt/mTOR pathway, including inhibitors of Akt and/or mTOR.

In some embodiments, an anti-cancer therapy of the disclosure comprises a PI3K inhibitor or Akt inhibitor, e.g., alone or in combination with a RET-targeted therapy. In some embodiments, the PI3K inhibitor inhibits one or more activities of PI3K. In some embodiments, the anti-cancer therapy/PI3K inhibitor is (a) a small molecule that inhibits one or more enzymatic activities of PI3K. (b) an antibody that inhibits one or more activities of PI3K (e.g., by binding to and inhibiting one or more activities of PI3K, binding to and inhibiting expression of PI3K, and/or binding to and inhibiting one or more activities of a cell expressing PI3K, such as by inducing antibody-dependent cellular cytotoxicity, ADCC, or phagocytosis, ADCP), or (c) a nucleic acid that inhibits expression of PI3K (e.g., an antisense oligonucleotide, miRNA, siRNA, morpholino, CRISPR-based therapeutic, and the like). In some embodiments, the PI3K inhibitor is a small molecule inhibitor of PI3K (e.g., a competitive or non-competitive inhibitor). Non-limiting examples of PI3K inhibitors include GSK2636771, buparlisib (BKM120), AZD8186, copanlisib (BAY80-6946), LY294002, PX-866, TGX115, TGX126, BEZ235, SF1126, idelalisib (GS-1101, CAL-101), pictilisib (GDC-094), GDC0032, IPI145, INK1117 (MLN1117), SAR260301, KIN-193 (AZD6482), duvelisib, GS-9820, GSK2636771, GDC-0980, AMG319, pazobanib, and alpelisib (BYL719, Piqray), as well as pharmaceutically acceptable salts thereof. In some embodiments, the AKT inhibitor inhibits one or more activities of AKT (e.g., AKT1). In some embodiments, the AKT inhibitor is (a) a small molecule that inhibits one or more enzymatic activities of AKT1, (b) an antibody that inhibits one or more activities of AKT1 (e.g., by binding to and inhibiting one or more activities of AKT1, binding to and inhibiting expression of AKT1, and/or binding to and inhibiting one or more activities of a cell expressing AKT1, such as by inducing antibody-dependent cellular cytotoxicity, ADCC, or phagocytosis, ADCP), or (c) a nucleic acid that inhibits expression of AKT1 (e.g., an antisense oligonucleotide, miRNA, siRNA, morpholino, CRISPR-based therapeutic, and the like). In some embodiments, the AKT1 inhibitor is a small molecule inhibitor of AKT1 (e.g., a competitive or non-competitive inhibitor). Non-limiting examples of AKT1 inhibitors include GSK690693, GSK2141795 (uprosertib), GSK2110183 (afuresertib), AZD5363, GDC-0068 (ipatasertib), AT7867, CCT128930, MK-2206, BAY 1125976, AKT1 and AKT2-IN-1, perifosine, and VIII, as well as pharmaceutically acceptable salts thereof. In some embodiments, the AKT1 inhibitor is a pan-Akt inhibitor.

In some embodiments, an anti-cancer therapy of the disclosure comprises a hedgehog (Hh) inhibitor, e.g., alone or in combination with a RET-targeted therapy. In some embodiments, the Hh inhibitor is (a) a small molecule that inhibits one or more enzymatic activities of Hh. (b) an antibody that inhibits one or more activities of Hh (e.g., by binding to and inhibiting one or more activities of Hh, binding to and inhibiting expression of Hh, and/or binding to and inhibiting one or more activities of a cell expressing Hh, such as by inducing antibody-dependent cellular cytotoxicity, ADCC, or phagocytosis, ADCP), or (c) a nucleic acid that inhibits expression of Hh (e.g., an antisense oligonucleotide, miRNA, siRNA, morpholino, CRISPR-based therapeutic, and the like). In some embodiments, the Hh inhibitor is a small molecule inhibitor of Hh (e.g., a competitive or non-competitive inhibitor). Non-limiting examples of Hh inhibitors include sonidegib, vismodegib, erismodegib, saridegib, BMS833923, PF-04449913, and LY2940680, as well as pharmaceutically acceptable salts thereof.

In some embodiments, an anti-cancer therapy of the disclosure comprises a heat shock protein (HSP) inhibitor, a MYC inhibitor, an HDAC inhibitor, an immunotherapy, a neoantigen, a vaccine, or a cellular therapy, e.g., alone or in combination with a RET-targeted therapy.

In some embodiments, the anti-cancer therapy comprises one or more of an immune checkpoint inhibitor, a chemotherapy, a VEGF inhibitor, an Integrin β3 inhibitor, a statin, an EGFR inhibitor, an mTOR inhibitor, a PI3K inhibitor, a MAPK inhibitor, or a CDK4/6 inhibitor, e.g., alone or in combination with a RET-targeted therapy.

In some embodiments, the anti-cancer therapy comprises a kinase inhibitor, e.g., alone or in combination with a RET-targeted therapy. In some embodiments, the kinase inhibitor is crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, or TAE684 (NVP-TAE684). In some embodiments, the kinase inhibitor is an ALK kinase inhibitor, e.g., as described in examples 3-39 of WO2005016894, which is incorporated herein by reference.

In some embodiments, the anti-cancer therapy comprises a heat shock protein (HSP) inhibitor, e.g., alone or in combination with a RET-targeted therapy. In some embodiments, the HSP inhibitor is a Pan-HSP inhibitor, such as KNK423. In some embodiments, the HSP inhibitor is an HSP70 inhibitor, such as cmHsp70.1, quercetin, VER155008, or 17-AAD. In some embodiments, the HSP inhibitor is a HSP90 inhibitor. In some embodiments, the HSP90 inhibitor is 17-AAD, Debio0932, ganetespib (STA-9090), retaspimycin hydrochloride (retaspimycin, IPI-504), AUY922, alvespimycin (KOS-1022, 17-DMAG), tanespimycin (KOS-953, 17-AAG), DS 2248, or AT13387 (onalespib). In some embodiments, the HSP inhibitor is an HSP27 inhibitor, such as Apatorsen (OGX-427).

In some embodiments, the anti-cancer therapy comprises a MYC inhibitor, e.g., alone or in combination with a RET-targeted therapy. In some embodiments, the MYC inhibitor is MYCi361 (NUCC-0196361), MYCi975 (NUCC-0200975), Omomyc (dominant negative peptide), ZINC16293153 (Min9), 10058-F4, JKY-2-169, 7594-0035, or inhibitors of MYC/MAX dimerization and/or MYC/MAX/DNA complex formation.

In some embodiments, the anti-cancer therapy comprises a histone deacetylase (HDAC) inhibitor, e.g., alone or in combination with a RET-targeted therapy. In some embodiments, the HDAC inhibitor is belinostat (PXD101, e.g., Beleodaq®), SAHA (vorinostat, suberoylanilide hydroxamine, e.g., Zolinza®), panobinostat (LBH589, LAQ-824), ACY1215 (Rocilinostat), quisinostat (JNJ-26481585), abexinostat (PCI-24781), pracinostat (SB939), givinostat (ITF2357), resminostat (4SC-201), trichostatin A (TSA), MS-275 (etinostat), Romidepsin (depsipeptide, FK228), MGCD0103 (mocetinostat), BML-210, CAY10603, valproic acid, MC1568, CUDC-907, CI-994 (Tacedinaline), Pivanex (AN-9), AR-42, Chidamide (CS055, HBI-8000), CUDC-101, CHR-3996, MPTOE028, BRD8430, MRLB-223, apicidin, RGFP966, BG45, PCI-34051, C149 (NCC149), TMP269, Cpd2, T247, T326, LMK235, CIA, HPOB, Nexturastat A, Befexamac, CBHA, Phenylbutyrate, MC1568, SNDX275, Scriptaid, Merck60, PX089344, PX105684, PX117735, PX117792, PX117245, PX105844, compound 12 as described by Li et al., Cold Spring Harb Perspect Med (2016) 6 (10): a026831, or PX117445.

In some embodiments, the anti-cancer therapy comprises a VEGF inhibitor, e.g., alone or in combination with a RET-targeted therapy. In some embodiments, the VEGF inhibitor is Bevacizumab (e.g., Avastin®), BMS-690514, ramucirumab, pazopanib, sorafenib, sunitinib, golvatinib, vandetanib, cabozantinib, levantinib, axitinib, cediranib, tivozanib, lucitanib, semaxanib, nindentanib, regorafinib, or aflibercept.

In some embodiments, the anti-cancer therapy comprises an integrin β3 inhibitor, e.g., alone or in combination with a RET-targeted therapy. In some embodiments, the integrin β3 inhibitor is anti-avb3 (clone LM609), cilengitide (EMD121974, NSC, 707544), an siRNA, GLPG0187, MK-0429. CNTO95, TN-161, etaracizumab (MEDI-522), intetumumab (CNTO95) (anti-alphaV subunit antibody), abituzumab (EMD 525797/DI17E6) (anti-alphaV subunit antibody), JSM6427, SJ749, BCH-15046, SCH221153, or SC56631. In some embodiments, the anti-cancer therapy comprises an αIIbβ3 integrin inhibitor, e.g., alone or in combination with a RET-targeted therapy. In some embodiments, the αIIbβ3 integrin inhibitor is abciximab, eptifibatide (e.g., Integrilin®), or tirofiban (e.g., Aggrastat®).

In some embodiments, the anti-cancer therapy comprises an mTOR inhibitor, e.g., alone or in combination with a RET-targeted therapy. In some embodiments, the mTOR inhibitor is temsirolimus (CCI-779), KU-006379, PP242, Torin1, Torin2, ICSN3250, Rapalink-1, CC-223, sirolimus (rapamycin), everolimus (RAD001), dactosilib (NVP-BEZ235), GSK2126458, WAY-001, WAY-600, WYE-687, WYE-354, SF1126, XL765, INK128 (MLN012), AZD8055, OSI027, AZD2014, or AP-23573.

In some embodiments, the anti-cancer therapy comprises a statin or a statin-based agent, e.g., alone or in combination with a RET-targeted therapy. In some embodiments, the statin or statin-based agent is simvastatin, atorvastatin, fluvastatin, pitavastatin, pravastatin, rosuvastatin, or cerivastatin.

In some embodiments, the anti-cancer therapy comprises a MAPK inhibitor, e.g., alone or in combination with a RET-targeted therapy. In some embodiments, the MAPK inhibitor is SB203580, SKF-86002, BIRB-796, SC-409, RJW-67657, BIRB-796, VX-745, RO3201195, SB-242235, or MW181.

In some embodiments, the anti-cancer therapy comprises an EGFR inhibitor, e.g., alone or in combination with a RET-targeted therapy. In some embodiments, the EGFR inhibitor is cetuximab, panitumumab, lapatinib, gefitinib, vandetanib, dacomitinib, icotinib, osimertinib (AZD9291), afatanib, olmutinib, EGF816 (nazartinib), avitinib (AC0010), rociletinib (CO-1686), BMS-690514, YH5448, PF-06747775, ASP8273, PF299804, AP26113, necitumumab (e.g., Portrazza®), or erlotinib. In some embodiments, the EGFR inhibitor is gefitinib or cetuximab.

In some embodiments, an anti-cancer therapy of the disclosure comprises a cancer immunotherapy, such as a checkpoint inhibitor, cancer vaccine, cell-based therapy, T cell receptor (TCR)-based therapy, adjuvant immunotherapy, cytokine immunotherapy, and oncolytic virus therapy, e.g., alone or in combination with a RET-targeted therapy. In some embodiments, the cancer immunotherapy comprises a small molecule, nucleic acid, polypeptide, carbohydrate, toxin, cell-based agent, or cell-binding agent. Examples of cancer immunotherapies are described in greater detail herein but are not intended to be limiting. In some embodiments, the cancer immunotherapy activates one or more aspects of the immune system to attack a cell (e.g., a tumor cell) that expresses a neoantigen, e.g., a neoantigen corresponding to a RET fusion nucleic acid molecule or polypeptide of the disclosure. The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises a cancer vaccine, e.g., alone or in combination with a RET-targeted therapy. A range of cancer vaccines have been tested that employ different approaches to promoting an immune response against a cancer (see, e.g., Emens L A, Expert Opin Emerg Drugs 13 (2): 295-308 (2008) and US20190367613). Approaches have been designed to enhance the response of B cells. T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello, A, et al, J. Clin. Invest. 95:341, 1995), naked cDNA or peptides, encapsulated e.g., in poly (DL-lactide-co-glycolide) (“PLG”) microspheres (see, e.g., Eldridge, et ah, Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tam, J. P., J. Immunol. Methods 196:17-32, 1996). In some embodiments, a cancer vaccine is formulated as a peptide-based vaccine, or nucleic acid based vaccine in which the nucleic acid encodes the polypeptides. In some embodiments, a cancer vaccine is formulated as an antibody-based vaccine. In some embodiments, a cancer vaccine is formulated as a cell based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al, Cancer Sci, 104:14-21, 2013). In some embodiments, such cancer vaccines augment the anti-cancer response.

In some embodiments, the cancer vaccine comprises a polynucleotide that encodes a neoantigen, e.g., a neoantigen corresponding to a RET fusion nucleic acid molecule or polypeptide of the disclosure. In some embodiments, the cancer vaccine comprises DNA that encodes a neoantigen. e.g., a neoantigen corresponding to a RET fusion nucleic acid molecule or polypeptide of the disclosure. In some embodiments, the cancer vaccine comprises RNA that encodes a neoantigen, e.g., a neoantigen corresponding to a RET fusion nucleic acid molecule or polypeptide of the disclosure. In some embodiments, the cancer vaccine comprises a polynucleotide that encodes a neoantigen, e.g., a neoantigen corresponding to a RET fusion nucleic acid molecule or polypeptide of the disclosure. In some embodiments, the cancer vaccine further comprises one or more additional antigens, neoantigens, or other sequences that promote antigen presentation and/or an immune response. In some embodiments, the polynucleotide is complexed with one or more additional agents, such as a liposome or lipoplex. In some embodiments, the polynucleotide(s) are taken up and translated by antigen presenting cells (APCs), which then present the neoantigen(s) via MHC class I on the APC cell surface.

In some embodiments, the cancer vaccine is selected from sipuleucel-T (e.g., Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (e.g., Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (PexaVec/JX-594, SillaJen/formerly Jennerex Biotherapeutics), a thymidine kinase-(TK-) deficient vaccinia virus engineered to express GM-CSF, for hepatocellular carcinoma (NCT02562755) and melanoma (NCT00429312); pelarcorep (e.g., Reolysin®, Oncolytics Biotech), a variant of respiratory enteric orphan virus (reovirus) which does not replicate in cells that are not RAS-activated, in numerous cancers, including colorectal cancer (NCT01622543), prostate cancer (NCT01619813), head and neck squamous cell cancer (NCT01166542), pancreatic adenocarcinoma (NCT00998322), and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117), metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676), and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260), fallopian tube cancer, ovarian cancer (NCT 02759588); or CG0070 (Cold Genesys), an adenovirus engineered to express GM-CSF in bladder cancer (NCT02365818); anti-gp100; STINGVAX; GVAX; DCVaxL; and DNX-2401. In some embodiments, the cancer vaccine is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFa-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into an individual (possibly with pro-inflammatory or other attractants such as GM-CSF), taken up by cells in vivo to make the specific antigens, which then provoke the desired immune response. In some embodiments, vectors may be used to deliver more than one tumor antigen at a time, to increase the immune response. In addition, recombinant virus, bacteria or yeast vectors can trigger their own immune responses, which may also enhance the overall immune response.

In some embodiments, the cancer vaccine comprises a DNA-based vaccine. In some embodiments, DNA-based vaccines can be employed to stimulate an anti-tumor response. The ability of directly injected DNA that encodes an antigenic protein, to elicit a protective immune response has been demonstrated in numerous experimental systems. Vaccination through directly injecting DNA that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12:771-776). In some embodiments, plasmid (or other vector) DNA that includes a sequence encoding a protein operably linked to regulatory elements required for gene expression is administered to individuals (e.g. human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen.

In some embodiments, the cancer immunotherapy comprises a cell-based therapy. In some embodiments, the cancer immunotherapy comprises a T cell-based therapy. In some embodiments, the cancer immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) cancer cells. In some embodiments, the immune response results in inhibition of tumor and/or metastatic cell growth and/or proliferation, and in related embodiments, results in neoplastic cell death and/or resorption. The immune cells can be derived from a different organism/host (exogenous immune cells) or can be cells obtained from the subject organism (autologous immune cells). In some embodiments, the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g. chimeric). In some embodiments, a population of immune cells can be obtained from a subject in need of therapy or suffering from a disease associated with reduced immune cell activity. Thus, the cells will be autologous to the subject in need of therapy. In some embodiments, a population of immune cells can be obtained from a donor, such as a histocompatibility-matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible, in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy, such as autologous cells, e.g., tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. Several approaches for the isolation, derivation, engineering or modification, activation, and expansion of functional anti-tumor effector cells have been described in the last two decades and may be used according to any of the methods provided herein. In some embodiments, the T cells are derived from the blood, bone marrow, lymph, umbilical cord, or lymphoid organs. In some embodiments, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs).

In some embodiments, the T cell-based therapy comprises a chimeric antigen receptor (CAR)-T cell-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen. In some embodiments, the CAR specifically binds a neoantigen, such as a neoantigen corresponding to a RET fusion nucleic acid molecule or polypeptide of the disclosure.

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen. In some embodiments, the recombinant TCR specifically binds a neoantigen corresponding to a RET fusion nucleic acid molecule or polypeptide of the disclosure.

In some embodiments, the T cell-based therapy comprises tumor-infiltrating lymphocytes (TILs). For example, TILs can be isolated from a tumor or cancer of the present disclosure, then isolated and expanded in vitro. Some or all of these TILs may specifically recognize an antigen expressed by the tumor or cancer of the present disclosure. In some embodiments, the TILs are exposed to one or more neoantigens, e.g., a neoantigen corresponding to a RET fusion nucleic acid molecule or polypeptide of the disclosure, in vitro after isolation. TILs are then administered to the patient (optionally in combination with one or more cytokines or other immune-stimulating substances).

In some embodiments, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells can be detected by specific surface markers, such as CD16, CD56, and CD8 in humans. NK cells do not express T-cell antigen receptors, the pan T marker CD3, or surface immunoglobulin B cell receptors. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art.

In some embodiments, the cell-based therapy comprises a dendritic cell (DC)-based therapy, e.g., a dendritic cell vaccine. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient. In some embodiments, immunization may be repeated multiple times if desired. Methods for harvesting, expanding, and administering dendritic cells are known in the art; see, e.g., WO2019178081. Dendritic cell vaccines (such as Sipuleucel-T, also known as APC8015 and PROVENGE®) are vaccines that involve administration of dendritic cells that act as APCs to present one or more cancer-specific antigens to the patient's immune system. In some embodiments, the dendritic cells are autologous or allogeneic to the recipient.

In some embodiments, the cancer immunotherapy comprises a TCR-based therapy. In some embodiments, the cancer immunotherapy comprises administration of one or more TCRs or TCR-based therapeutics that specifically bind an antigen expressed by a cancer of the present disclosure, e.g., a neoantigen corresponding to a RET fusion nucleic acid molecule or polypeptide of the disclosure. In some embodiments, the TCR-based therapeutic may further include a moiety that binds an immune cell (e.g., a T cell), such as an antibody or antibody fragment that specifically binds a T cell surface protein or receptor (e.g., an anti-CD3 antibody or antibody fragment).

In some embodiments, the immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the immunotherapy comprises cytokine immunotherapy. Cytokine immunotherapy comprises the use of one or more cytokines that activate components of the immune system. Examples include, but are not limited to, aldesleukin (e.g., PROLEUKIN®; interleukin-2), interferon alfa-2a (e.g., ROFERON®-A), interferon alfa-2b (e.g., INTRON®-A), and peginterferon alfa-2b (e.g., PEGINTRON®).

In some embodiments, the immunotherapy comprises oncolytic virus therapy. Oncolytic virus therapy uses genetically modified viruses to replicate in and kill cancer cells, leading to the release of antigens that stimulate an immune response. In some embodiments, replication-competent oncolytic viruses expressing a tumor antigen comprise any naturally occurring (e.g., from a “field source”) or modified replication-competent oncolytic virus. In some embodiments, the oncolytic virus, in addition to expressing a tumor antigen, may be modified to increase selectivity of the virus for cancer cells. In some embodiments, replication-competent oncolytic viruses include, but are not limited to, oncolytic viruses that are a member in the family of myoviridae, siphoviridae, podpviridac, teciviridae, corticoviridae, plasmaviridae, lipothrixviridae, fuselloviridae, poxyiridac, iridoviridac, phycodnaviridae, baculoviridae, herpesviridae, adnoviridae, papovaviridae, polydnaviridac, inoviridae, microviridae, geminiviridae, circoviridae, parvoviridae, hepadnaviridae, retroviridac, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridac, caliciviridae, astroviridae, nodaviridae, tetraviridae, tombusviridae, coronaviridae, glaviviridae, togaviridae, and barnaviridae. In some embodiments, replication-competent oncolytic viruses include adenovirus, retrovirus, reovirus, rhabdovirus, Newcastle Discase virus (NDV), polyoma virus, vaccinia virus (VacV), herpes simplex virus, picornavirus, coxsackie virus and parvovirus. In some embodiments, a replicative oncolytic vaccinia virus expressing a tumor antigen may be engineered to lack one or more functional genes in order to increase the cancer selectivity of the virus. In some embodiments, an oncolytic vaccinia virus is engineered to lack thymidine kinase (TK) activity. In some embodiments, the oncolytic vaccinia virus may be engineered to lack vaccinia virus growth factor (VGF). In some embodiments, an oncolytic vaccinia virus may be engineered to lack both VGF and TK activity. In some embodiments, an oncolytic vaccinia virus may be engineered to lack one or more genes involved in evading host interferon (IFN) response such as E3L, K3L, B18R, or B8R. In some embodiments, a replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen may be locally or systemically administered to a subject, e.g. via intratumoral, intraperitoneal, intravenous, intra-arterial, intramuscular, intradermal, intracranial, subcutaneous, or intranasal administration.

In some embodiments, an anti-cancer therapy of the disclosure comprises an immune checkpoint inhibitor, e.g., alone or in combination with a RET-targeted therapy. In some embodiments, the methods provided herein comprise administering to an individual an effective amount of an immune checkpoint inhibitor. As is known in the art, a checkpoint inhibitor targets at least one immune checkpoint protein to alter the regulation of an immune response. Immune checkpoint proteins include, e.g., CTLA4, PD-L1, PD-1, PD-L2, VISTA, B7-H2, B7-H3, B7-H4, B7-H6, 2B4, ICOS, HVEM, CEACAM, LAIR1, CD80, CD86, CD276, VTCN1, MHC class I, MHC class II, GALS, adenosine, TGFR, CSF1R, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4. TIGIT, LAG-3, BTLA, IDO, OX40, and A2aR. In some embodiments, molecules involved in regulating immune checkpoints include, but are not limited to: PD-1 (CD279), PD-L1 (B7-H1, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GAL9, VISTA (PD-1H), TIGIT, LAIR1, CD160, 2B4, TGFRbeta, A2AR, GITR (CD357), CD80 (B7-1), CD86 (B7-2), CD276 (B7-H3), VTCNI (B7-H4), MHC class I, MHC class II, GALS, adenosine, TGFR, B7-H1, OX40 (CD134), CD94 (KLRD1), CD137 (4-1BB), CD137L (4-1BBL), CD40, IDO, CSF1R, CD40L, CD47, CD70 (CD27L), CD226, HHLA2, ICOS (CD278), ICOSL (CD275), LIGHT (TNFSF14, CD258), NKG2a, NKG2d, OX40L (CD134L), PVR (NECL5, CD155), SIRPa, MICA/B, and/or arginase. In some embodiments, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. Examples of checkpoint inhibitors include, without limitation, a PD-1 axis binding antagonist, a PD-L1 axis binding antagonist (e.g., an anti-PD-L1 antibody, e.g., atczolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or antibodies, such as human antibodies (scc, e.g., International Patent Publication WO2015016718; Pardoll, Nat Rev Cancer, 12 (4): 252-64, 2012; both incorporated herein by reference). In some embodiments, known inhibitors of immune checkpoint proteins or analogs thereof may be used, in particular chimerized, humanized or human forms of antibodies may be used.

In some embodiments, the checkpoint inhibitor is a PD-L1 axis binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “B7-H,” and “PDL1.” An exemplary human PD-L1 is shown in UniProtKB/Swiss-Prot Accession No.Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some instances, the PD-1 binding antagonist is a molecule that inhibits the binding of PD-1 to its ligand binding partners. In a specific embodiment, the PD-1 ligand binding partners are PD-L1 and/or PD-L2. In another instance, a PD-L1 binding antagonist is a molecule that inhibits the binding of PD-L1 to its binding ligands. In a specific embodiment, PD-L1 binding partners are PD-1 and/or B7-1. In another instance, the PD-L2 binding antagonist is a molecule that inhibits the binding of PD-L2 to its ligand binding partners. In a specific embodiment, the PD-L2 binding ligand partner is PD-1. The antagonist may be an antibody, an antigen binding fragment thereof, an immunoadhesin, a fusion protein, or an oligopeptide. In some embodiments, the PD-1 binding antagonist is a small molecule, a nucleic acid, a polypeptide (e.g., antibody), a carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is one or more of MDX-1 106 (nivolumab). MK-3475 (pembrolizumab, e.g., Keytruda®), MEDI-0680 (AMP-514), PDR001, REGN2810, MGA-012, JNJ-63723283. BI 754091, or BGB-108. In other instances, the PD-1 binding antagonist is an immunoadhesin (e.g., an immunoadhesin comprising an extracellular or PD-1 binding portion of PD-L1 or PD-L2 fused to a constant region (e.g., an Fe region of an immunoglobulin sequence)). In some instances, the PD-1 binding antagonist is AMP-224. Other examples of anti-PD-1 antibodies include, but are not limited to, MEDI-0680 (AMP-514; AstraZeneca), PDR001 (CAS Registry No. 1859072-53-9; Novartis), REGN2810 (e.g., LIBTAYO® or cemiplimab-rwlc; Regeneron). BGB-108 (BeiGene), BGB-A317 (BeiGene), BI 754091, JS-001 (Shanghai Junshi), STI-A1110 (Sorrento). INCSHR-1210 (Incytc), PF-06801591 (Pfizer), TSR-042 (also known as ANB011; Tesaro/AnaptysBio), AM0001 (ARMO Biosciences), ENUM 244C8 (Enumeral Biomedical Holdings), or ENUM 388D4 (Enumeral Biomedical Holdings). In some embodiments, the PD-1 axis binding antagonist comprises tislelizumab (BGB-A317), BGB-108, STI-A1110, AM0001, BI 754091, sintilimab (IBI308), cetrelimab (JNJ-63723283), toripalimab (JS-001), camrelizumab (SHR-1210. INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, e.g., Keytruda®), PF-06801591, cemiplimab (REGN-2810, REGEN2810), dostarlimab (TSR-042, ANB011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, or CCX-4503, or derivatives thereof.

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In some embodiments, the PD-L1 binding antagonist is an anti-PD-L1 antibody. In some embodiments, the anti-PD-L1 antibody can bind to a human PD-L1, for example a human PD-L1 as shown in UniProtKB/Swiss-Prot Accession No.Q9NZQ7.1, or a variant thereof. In some embodiments, the PD-L1 binding antagonist is a small molecule, a nucleic acid, a polypeptide (e.g., antibody), a carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting the binding between PD-L1 and PD-1, and/or between PD-L1 and B7-1. In some instances, the anti-PD-L1 antibody is a monoclonal antibody. In some instances, the anti-PD-L1 antibody is an antibody fragment selected from a Fab, Fab′-SH, Fv, scFv, or (Fab′)2 fragment. In some instances, the anti-PD-L1 antibody is a humanized antibody. In some instances, the anti-PD-L1 antibody is a human antibody. In some instances, the anti-PD-L1 antibody is selected from YW243.55.S70, MPDL3280A (atczolizumab), MDX-1 105, MEDI4736 (durvalumab), or MSB0010718C (avelumab). In some embodiments, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311. AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, or FS-118, or a derivative thereof.

In some embodiments, the checkpoint inhibitor is an antagonist of CTLA4. In some embodiments, the checkpoint inhibitor is a small molecule antagonist of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist is a small molecule, a nucleic acid, a polypeptide (e.g., antibody), a carbohydrate, a lipid, a metal, or a toxin. In some embodiments, the CTLA-4 inhibitor comprises ipilimumab (IBI310, BMS-734016, MDX010, MDX-CTLA4, MEDI4736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof.

In some embodiments, the anti-PD-1 antibody or antibody fragment is MDX-1106 (nivolumab), MK-3475 (pembrolizumab, e.g., Keytruda®), MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the PD-1 binding antagonist is an anti-PD-1 immunoadhesin. In some embodiments, the anti-PD-1 immunoadhesin is AMP-224. In some embodiments, the anti-PD-L1 antibody or antibody fragment is YW243.55.S70, MPDL3280A (atczolizumab), MDX-1105, MEDI4736 (durvalumab), MSB0010718C (avelumab), LY3300054, STI-A1014, KN035, FAZ053, or CX-072.

In some embodiments, the immune checkpoint inhibitor comprises a LAG-3 inhibitor (e.g., an antibody, an antibody conjugate, or an antigen-binding fragment thereof). In some embodiments, the LAG-3 inhibitor comprises a small molecule, a nucleic acid, a polypeptide (e.g., an antibody), a carbohydrate, a lipid, a metal, or a toxin. In some embodiments, the LAG-3 inhibitor comprises a small molecule. In some embodiments, the LAG-3 inhibitor comprises a LAG-3 binding agent. In some embodiments, the LAG-3 inhibitor comprises an antibody, an antibody conjugate, or an antigen-binding fragment thereof. In some embodiments, the LAG-3 inhibitor comprises cftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceding.

In some embodiments, an anti-cancer therapy of the disclosure comprises an immunoregulatory molecule or a cytokine, e.g., alone or in combination with a RET-targeted therapy. An immunoregulatory profile is required to trigger an efficient immune response and balance the immunity in a subject. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, gIp10, TCA-3, MCP-1, MIF, MIP-1α, MIP-1β, Rantes, macrophage colony stimulating factor (M-CSF), granulocyte colony stimulating factor (G-CSF), or granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present disclosure. Examples of chemokines include, but are not limited to, MIP-3a (Lax), MIP-3B, Hcc-1, MPIF-1, MPIF-2, MCP-2, MCP-3, MCP-4, MCP-5, Eotaxin, Tarc, Elc, 1309, IL-8, GCP-2 Groα, Gro-β, Nap-2, Ena-78, Ip-10, MIG, I-Tac, SDF-1, or BCA-1 (Blc), as well as functional fragments thereof. In some embodiments, the immunoregulatory molecule is included with any of the treatments provided herein.

In some embodiments, the immune checkpoint inhibitor is monovalent and/or monospecific. In some embodiments, the immune checkpoint inhibitor is multivalent and/or multispecific.

In some embodiments, an anti-cancer therapy of the disclosure comprises an anti-cancer agent that inhibits expression of a nucleic acid that comprises or encodes a RET fusion nucleic acid molecule of the disclosure or a portion thereof, or a RET fusion polypeptide of the disclosure, or a portion thereof. In some embodiments, the anti-cancer therapy comprises a nucleic acid molecule, such as a dsRNA, an siRNA, or an shRNA. As is known in the art, dsRNAs having a duplex structure are effective at inducing RNA interference (RNAi). In some embodiments, the anti-cancer therapy comprises a small interfering RNA molecule (siRNA). dsRNAs and siRNAs can be used to silence gene expression in mammalian cells (e.g., human cells). In some embodiments, a dsRNA of the disclosure comprises any of between about 5 and about 10 base pairs, between about 10 and about 12 base pairs, between about 12 and about 15 base pairs, between about 15 and about 20 base pairs, between about 20 and 23 base pairs, between about 23 and about 25 base pairs, between about 25 and about 27 base pairs, or between about 27 and about 30 base pairs. As is known in the art, siRNAs are small dsRNAs that optionally include overhangs. In some embodiments, the duplex region of an siRNA is between about 18 and 25 nucleotides, e.g., any of 18, 19, 20, 21, 22, 23, 24, or 25 nucleotides. siRNAs may also include short hairpin RNAs (shRNAs), e.g., with approximately 29-base-pair stems and 2-nucleotide 3′ overhangs. In some embodiments, a dsRNA, an siRNA, or an shRNA of the disclosure comprises a nucleotide sequence that is configured to hybridize to a nucleic acid that comprises or encodes a RET fusion nucleic acid molecule of the disclosure or a portion thereof comprising a breakpoint. Methods for designing, optimizing, producing, and using dsRNAs, siRNAs, or shRNAs, are known in the art.

In some embodiments, an anti-cancer therapy of the disclosure comprises a chemotherapy, e.g., alone or in combination with a RET-targeted therapy. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trictylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprinc, and thioguanine; pyrimidine analogs, such as ancitabine, azacitidine, 6-azauridinc, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; cdatraxate; defofamine; demecolcine; diaziquone; elformithine; elliptinium acetate; an cpothilone; ctoglucid; gallium nitrate; hydroxyurca; lentinan; lonidainine; maytansinoids, such as maytansine and ansamitocins; mitoguazone; mitoxantrone; mopidanmol; nitracrine; pentostatin; phenamet; pirarubicin; losoxantrone; podophyllinic acid; 2-ethylhydrazide; procarbazine; PSK polysaccharide complex: razoxane: rhizoxin: sizofiran: spirogermanium: tenuazonic acid: triaziquone: 2,2′,2″-trichlorotricthylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”): cyclophosphamide: taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabine, navelbine, famesyl-protein tansferase inhibitors, transplatinum, and pharmaceutically acceptable salts, acids, or derivatives of any of the above.

Some non-limiting examples of chemotherapeutic drugs which can be combined with anti-cancer therapies of the present disclosure are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).

In some embodiments, an anti-cancer therapy of the disclosure comprises a kinase inhibitor, e.g., alone or in combination with a RET-targeted therapy. Examples of kinase inhibitors include those that target one or more receptor tyrosine kinases, e.g., BCR-ABL, B-Raf, EGFR, HER-2/ErbB2, IGF-IR, PDGFR-a, PDGFR-β, cKit, Flt-4, Flt3, FGFR1, FGFR2, FGFR3, FGFR4, CSF1R, c-Met, ROS1, RON, c-Ret, or ALK; one or more cytoplasmic tyrosine kinases, e.g., c-SRC, c-YES, Abl, or JAK-2; one or more serine/threonine kinases, e.g., ATM, Aurora A & B, CDKs, mTOR, PKCi, PLKs, b-Raf, c-Raf, S6K, or STK11/LKB1; or one or more lipid kinases, e.g., PI3K or SKI. Small molecule kinase inhibitors include PHA-739358, nilotinib, dasatinib, PD166326, NSC 743411, lapatinib (GW-572016), canertinib (CI-1033), semaxinib (SU5416), vatalanib (PTK787/ZK222584), sutent (SU1 1248), sorafenib (BAY 43-9006), or leflunomide (SU101). Additional non-limiting examples of tyrosine kinase inhibitors include imatinib (Gleevec/Glivec) and gefitinib (Iressa).

In some embodiments, an anti-cancer therapy of the disclosure comprises any of abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Belcodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcctris), brexucabtagene autolcucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cctuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), clotuzumab (Empliciti), enasidenib mesylate (Idhifa), encorafenib (Braftovi), enfortumab vedotin-cjfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane 1131 (Azedra), ipilimumab (Yervoy), isatuximab-irfc (Sarclisa), ivosidenib (Tibsovo), ixazomib citrate (Ninlaro), lanrcotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirinc-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligco), moxctumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pcmazyrc), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof, e.g., alone or in combination with a RET-targeted therapy.

In some embodiments, an anti-cancer therapy of the disclosure comprises an anti-angiogenic agent, e.g., alone or in combination with a RET-targeted therapy. Angiogenesis inhibitors prevent the extensive growth of blood vessels (angiogenesis) that tumors require to survive. Non-limiting examples of angiogenesis-mediating molecules or angiogenesis inhibitors which may be used in the methods of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-β and IFN-γ, CXCL10, IL-4, IL-12 and IL-18, prothrombin (kringle domain-2), antithrombin III fragment, prolactin. VEGI, SPARC, osteopontin, maspin, canstatin, proliferin-related protein, restin and drugs such as bevacizumab, itraconazole, carboxyamidotriazole, TNP-470, CM101, IFN-α platelet factor-4, suramin, SU5416, thrombospondin. VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina v β3 inhibitors, linomide, or tasquinimod. In some embodiments, known therapeutic candidates that may be used according to the methods of the disclosure include naturally occurring angiogenic inhibitors, including without limitation, angiostatin, endostatin, or platelet factor-4. In another embodiment, therapeutic candidates that may be used according to the methods of the disclosure include, without limitation, specific inhibitors of endothelial cell growth, such as TNP-470, thalidomide, and interleukin-12. Still other anti-angiogenic agents that may be used according to the methods of the disclosure include those that neutralize angiogenic molecules, including without limitation, antibodies to fibroblast growth factor, antibodies to vascular endothelial growth factor, antibodies to platelet derived growth factor, or antibodies or other types of inhibitors of the receptors of EGF, VEGF or PDGF. In some embodiments, anti-angiogenic agents that may be used according to the methods of the disclosure include, without limitation, suramin and its analogs, and tecogalan. In other embodiments, anti-angiogenic agents that may be used according to the methods of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used according to the methods of the disclosure includes, without limitation, anti-adhesion molecules, such as antibodies to integrin alpha v beta 3. Still other anti-angiogenic compounds or compositions that may be used according to the methods of the disclosure include, without limitation, kinase inhibitors, thalidomide, itraconazole, carboxyamidotriazole, CM101, IFN-α. IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound that may be used according to the methods of the disclosure is an antibody to VEGF, such as Avastin®/bevacizumab (Genentech).

In some embodiments, an anti-cancer therapy of the disclosure comprises an anti-DNA repair therapy, e.g., alone or in combination with a RET-targeted therapy. In some embodiments, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026).

In some embodiments, an anti-cancer therapy of the disclosure comprises a radiosensitizer, e.g., alone or in combination with a RET-targeted therapy. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors.

In some embodiments, an anti-cancer therapy of the disclosure comprises an anti-inflammatory agent, e.g., alone or in combination with a RET-targeted therapy. In some embodiments, the anti-inflammatory agent is an agent that blocks, inhibits, or reduces inflammation or signaling from an inflammatory signaling pathway. In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23: interferons (IFNs), e.g., IFNα, IFNβ, IFNγ, IFN-γ inducing factor (IGIF): transforming growth factor-β (TGF-β); transforming growth factor-α (TGF-α): tumor necrosis factors, e.g., TNF-α, TNF-β, TNF-RI, TNF-RII; CD23; CD30; CD40L; EGF; G-CSF; GDNF; PDGF-BB; RANTES/CCL5; IKK; NF-κB: TLR2; TLR3; TLR4; TL5; TLR6; TLR7; TLR8; TLR8; TLR9; and/or any cognate receptors thereof. In some embodiments, the anti-inflammatory agent is an IL-1 or IL-1 receptor antagonist, such as anakinra (e.g., Kineret®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (e.g., ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (Remicade R), golimumab (Simponi R), adalimumab (e.g., Humira®), certolizumab pegol (e.g., Cimzia®) or etanercept. In some embodiments, the anti-inflammatory agent is a corticosteroid. Exemplary corticosteroids include, but are not limited to, cortisone (hydrocortisone, hydrocortisone sodium phosphate, hydrocortisone sodium succinate, e.g., Ala-Cort®, Hydrocort Acetate®, hydrocortone phosphate Lanacort®, Solu-Cortef®), decadron (dexamethasone, dexamethasone acetate, dexamethasone sodium phosphate, e.g., Dexasone®, Diodex®, Hexadrol®, Maxidex®), methylprednisolone (6-methylprednisolone, methylprednisolone acetate, methylprednisolone sodium succinate, e.g., Duralone®, Medralone®, Medrol®, M-Prednisol®, Solu-Medrol®), prednisolone (e.g., Delta-Cortef®, ORAPRED®, Pediapred®, Prezone®), and prednisone (e.g., Deltasone®, Liquid Pred®, Meticorten®, Orasone®), and bisphosphonates (e.g., pamidronate (Aredia®), and zoledronic acid (e.g., Zometac®).

In some embodiments, an anti-cancer therapy of the disclosure comprises an anti-hormonal agent, e.g., alone or in combination with a RET-targeted therapy. Anti-hormonal agents are agents that act to regulate or inhibit hormone action on tumors. Examples of anti-hormonal agents include anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4 (5)-imidazoles, aminoglutethimide, MEGACE® megestrol acetate, AROMASIN® exemestane, formestanic, fadrozole, RIVISOR® vorozole, FEMARA® letrozole, and ARIMIDEX® (anastrozole); anti-androgens such as flutamide, nilutamide, bicalutamide, leuprolide, and goserelin; troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In some embodiments, an anti-cancer therapy of the disclosure comprises an antimetabolite chemotherapeutic agent, e.g., alone or in combination with a RET-targeted therapy. Antimetabolite chemotherapeutic agents are agents that are structurally similar to a metabolite, but cannot be used by the body in a productive manner. Many antimetabolite chemotherapeutic agents interfere with the production of RNA or DNA. Examples of antimetabolite chemotherapeutic agents include gemcitabine (e.g., GEMZAR®), 5-fluorouracil (5-FU), capecitabine (e.g., XELODATM), 6-mercaptopurine, methotrexate, 6-thioguanine, pemetrexed, raltitrexed, arabinosylcytosine ARA-C cytarabinc (e.g., CYTOSAR-U®), dacarbazine (DTIC-DOMED), azocytosine, deoxycytosine, pyridmidene, fludarabine (e.g., FLUDARA®), cladrabine, and 2-deoxy-D-glucose. In some embodiments, an antimetabolite chemotherapeutic agent is gemcitabine. Gemcitabine HCL is sold by Eli Lilly under the trademark GEMZAR®.

In some embodiments, an anti-cancer therapy of the disclosure comprises a platinum-based chemotherapeutic agent, e.g., alone or in combination with a RET-targeted therapy. Platinum-based chemotherapeutic agents are chemotherapeutic agents that comprise an organic compound containing platinum as an integral part of the molecule. In some embodiments, a chemotherapeutic agent is a platinum agent. In some such embodiments, the platinum agent is selected from cisplatin, carboplatin, oxaliplatin, nedaplatin, triplatin tetranitrate, phenanthriplatin, picoplatin, or satraplatin.

In some aspects, provided herein are therapeutic formulations comprising an anti-cancer therapy provided herein (e.g., a RET-targeted therapy, and/or any other anti-cancer therapy provided herein), and pharmaceutically acceptable carriers, excipients, or stabilizers. A formulation provided herein may contain more than one active compound, e.g., an anti-cancer therapy provided herein and one or more additional agents (e.g., anti-cancer agents).

Acceptable carriers, excipients, or stabilizers are non-toxic to recipients at the dosages and concentrations employed, and include, for example, one or more of: buffers such as phosphate, citrate, and other organic acids; antioxidants, including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, or m-cresol; low molecular weight polypeptides (e.g., less than about 10 residues); proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); surfactants such as non-ionic surfactants; or polymers such as polyethylene glycol (PEG).

The active ingredients may be entrapped in microcapsules. Such microcapsules may be prepared, for example, by coacervation techniques or by interfacial polymerization, for example, hydroxymethylcellulose or gelatin-microcapsules and poly-(methylmethacylate) microcapsules, respectively; in colloidal drug delivery systems (for example, liposomes, albumin microspheres, microemulsions, nano-particles and nano-capsules); or in macroemulsions. Such techniques are known in the art.

Sustained-release compositions may be prepared. Suitable examples of sustained-release compositions include semi-permeable matrices of solid hydrophobic polymers containing an anti-cancer therapy of the disclosure. Such matrices may be in the form of shaped articles, e.g., films, or microcapsules. Examples of sustained-release matrices include polyesters, hydrogels (for example, poly (2-hydroxyethyl-methacrylate), or poly (vinylalcohol)), polylactides, copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

A formulation provided herein may also contain more than one active compound, for example, those with complementary activities that do not adversely affect each other. The type and effective amounts of such medicaments depend, for example, on the amount and type of active compound(s) present in the formulation, and clinical parameters of the subjects.

For general information concerning formulations, see, e.g., Gilman et al. (eds.) The Pharmacological Bases of Therapeutics, 8th Ed., Pergamon Press, 1990; A. Gennaro (ed.), Remington's Pharmaceutical Sciences, 18th Edition, Mack Publishing Co., Pennsylvania, 1990; Avis et al. (eds.) Pharmaceutical Dosage Forms: Parenteral Medications Dekker, New York, 1993; Lieberman et al. (eds.) Pharmaceutical Dosage Forms: Tablets Dekker, New York, 1990; Lieberman et al. (eds.), Pharmaceutical Dosage Forms: Disperse Systems Dekker, New York, 1990; and Walters (ed.) Dermatological and Transdermal Formulations (Drugs and the Pharmaceutical Sciences), Vol 1 19, Marcel Dekker, 2002.

Formulations to be used for in vivo administration are sterile. This is readily accomplished by filtration through sterile filtration membranes or other methods known in the art.

In some embodiments, an anti-cancer therapy of the disclosure (e.g., a RET-targeted therapy) is administered as a monotherapy. In some embodiments, the anti-cancer therapy is administered in combination with one or more additional anti-cancer therapies or treatments, e.g., as described herein.

In some embodiments, the one or more additional anti-cancer therapies or treatments include one or more anti-cancer therapies described herein. In some embodiments, the methods of the present disclosure comprise administration of any combination of any of the anti-cancer therapies provided herein. In some embodiments, the additional anti-cancer therapy comprises one or more of surgery, radiotherapy, chemotherapy, anti-angiogenic therapy, anti-DNA repair therapy, and anti-inflammatory therapy. In some embodiments, the additional anti-cancer therapy comprises an anti-neoplastic agent, a chemotherapeutic agent, a growth inhibitory agent, an anti-angiogenic agent, a radiation therapy, a cytotoxic agent, or combinations thereof. In some embodiments, an anti-cancer therapy may be administered in conjunction with a chemotherapy or chemotherapeutic agent. In some embodiments, the chemotherapy or chemotherapeutic agent is a platinum-based agent (including, without limitation cisplatin, carboplatin, oxaliplatin, and staraplatin). In some embodiments, an anti-cancer therapy may be administered in conjunction with a radiation therapy.

### D. Reporting

In some embodiments, the methods provided herein comprise generating a report, and/or providing a report to party.

In some embodiments, a report according to the present disclosure comprises information about one or more of: a RET fusion nucleic acid molecule or a RET fusion polypeptide of the disclosure (e.g., any of the RET fusion nucleic acid molecules or RET fusion polypeptides described above and/or in the Examples herein); a cancer of the disclosure, e.g., comprising a RET fusion nucleic acid molecule or a RET fusion polypeptide of the disclosure; or a treatment, a therapy, or one or more treatment options for an individual having a cancer, such as a cancer of the disclosure (e.g., comprising a RET fusion nucleic acid molecule or a RET fusion polypeptide described herein).

In some embodiments, a report according to the present disclosure comprises information about the presence or absence of a RET fusion nucleic acid molecule or a RET fusion polypeptide of the disclosure in a sample obtained from an individual, such as an individual having a cancer, e.g., a cancer provided herein. In one embodiment, a report according to the present disclosure indicates that a RET fusion nucleic acid molecule or a RET fusion polypeptide of the disclosure is present in a sample obtained from the individual. In one embodiment, a report according to the present disclosure indicates that a RET fusion nucleic acid molecule or a RET fusion polypeptide of the disclosure is not present in a sample obtained from the individual. In one embodiment, a report according to the present disclosure indicates that a RET fusion nucleic acid molecule or a RET fusion polypeptide of the disclosure has been detected in a sample obtained from the individual. In one embodiment, a report according to the present disclosure indicates that a RET fusion nucleic acid molecule or a RET fusion polypeptide of the disclosure has not been detected in a sample obtained from the individual. In some embodiments, the report comprises an identifier for the individual from which the sample was obtained.

In some embodiments, the report includes information on the role of a RET fusion nucleic acid molecule or a RET fusion polypeptide of the disclosure in disease, such as in cancer. Such information can include one or more of: information on prognosis of a cancer, such as a cancer provided herein, e.g., comprising a RET fusion nucleic acid molecule or a RET fusion polypeptide described herein; information on resistance of a cancer, such as a cancer provided herein, e.g., comprising a RET fusion nucleic acid molecule or a RET fusion polypeptide described herein, to one or more treatments; information on potential or suggested therapeutic options (e.g., such as an anti-cancer therapy provided herein, or a treatment selected or identified according to the methods provided herein); or information on therapeutic options that should be avoided. In some embodiments, the report includes information on the likely effectiveness, acceptability, and/or advisability of applying a therapeutic option (e.g., such as an anti-cancer therapy provided herein, or a treatment selected or identified according to the methods provided herein) to an individual having a cancer, such as a cancer provided herein, e.g., comprising a RET fusion nucleic acid molecule or a RET fusion polypeptide described herein and identified in the report. In some embodiments, the report includes information or a recommendation on the administration of a treatment (e.g., an anti-cancer therapy provided herein, or a treatment selected or identified according to the methods provided herein). In some embodiments, the information or recommendation includes the dosage of the treatment and/or a treatment regimen (e.g., in combination with other treatments, such as a second therapeutic agent). In some embodiments, the report comprises information or a recommendation for at least one, at least two, at least three, at least four, at least five, at least six, at least seven, at least eight, at least nine, at least ten, or more treatments.

Also provided herein are methods of generating a report according to the present disclosure. In some embodiments, a report according to the present disclosure is generated by a method comprising one or more of the following steps: obtaining a sample, such as a sample described herein, from an individual, e.g., an individual having a cancer, such as a cancer provided herein; detecting a RET fusion nucleic acid molecule or a RET fusion polypeptide of the disclosure in the sample, or acquiring knowledge of the presence of the RET fusion nucleic acid molecule or RET fusion polypeptide of the disclosure in the sample; and generating a report. In some embodiments, a report generated according to the methods provided herein comprises one or more of: information about the presence or absence of a RET fusion nucleic acid molecule or a RET fusion polypeptide of the disclosure in the sample; an identifier for the individual from which the sample was obtained; information on the role of the RET fusion nucleic acid molecule or RET fusion polypeptide of the disclosure, or its wild type counterparts, in disease (e.g., such as in cancer); information on prognosis, resistance, or potential or suggested therapeutic options (such as an anti-cancer therapy provided herein, or a treatment selected or identified according to the methods provided herein); information on the likely effectiveness, acceptability, or the advisability of applying a therapeutic option (such as an anti-cancer therapy provided herein, or a treatment selected or identified according to the methods provided herein) to the individual; a recommendation or information on the administration of a treatment (such as an anti-cancer therapy provided herein, or a treatment selected or identified according to the methods provided herein); or a recommendation or information on the dosage or treatment regimen of a treatment (such as an anti-cancer therapy provided herein, or a treatment selected or identified according to the methods provided herein), e.g., in combination with other treatments (e.g., a second therapeutic agent). In some embodiments, the report generated is a personalized cancer report.

A report according to the present disclosure may be in an electronic, web-based, or paper form. The report may be provided to an individual or a patient (e.g., an individual or a patient having, suspected of having, or being tested for a cancer, such as a cancer provided herein, e.g., comprising a RET fusion nucleic acid molecule or a RET fusion polypeptide of the disclosure), or to an individual or entity other than the individual or patient, such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from the individual. In some embodiments, the report is provided or delivered to an individual or entity within any of about 1 day or more, about 7 days or more, about 14 days or more, about 21 days or more, about 30 days or more, about 45 days or more, or about 60 days or more from detecting a RET fusion nucleic acid molecule or a RET fusion polypeptide of the disclosure in a sample obtained from the individual. In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of a RET fusion nucleic acid molecule or a RET fusion polypeptide of the disclosure in a sample obtained from the individual.

### E. Software, Systems, and Devices

In some other aspects, provided herein are non-transitory computer-readable storage media. In some embodiments, the non-transitory computer-readable storage media comprise one or more programs for execution by one or more processors of a device, the one or more programs including instructions which, when executed by the one or more processors, cause the device to perform a method according to any of the embodiments described herein.

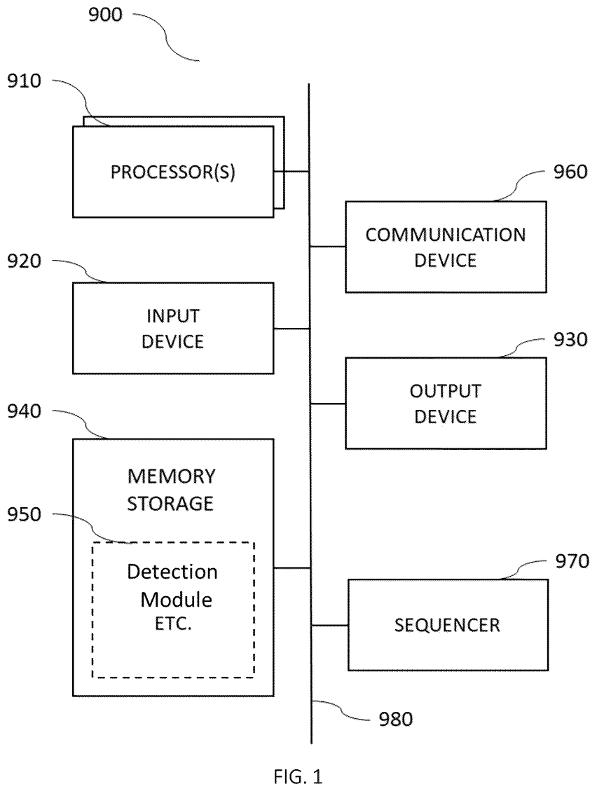

FIG. 1 illustrates an example of a computing device or system in accordance with one embodiment. Device 900 can be a host computer connected to a network. Device 900 can be a client computer or a server. As shown in FIG. 1, device 900 can be any suitable type of microprocessor-based device, such as a personal computer, workstation, server or handheld computing device (portable electronic device) such as a phone or tablet. The device can include, for example, one or more processor(s) 910, input devices 920, output devices 930, memory or storage devices 940, communication devices 960, and nucleic acid sequencers 970. Software 950 residing in memory or storage device 940 may comprise, e.g., an operating system as well as software for executing the methods described herein, e.g., for detecting a RET fusion nucleic acid molecule of the disclosure. Input device 920 and output device 930 can generally correspond to those described herein, and can cither be connectable or integrated with the computer.

Input device 920 can be any suitable device that provides input, such as a touch screen, keyboard or keypad, mouse, or voice-recognition device. Output device 930 can be any suitable device that provides output, such as a touch screen, haptics device, or speaker.

Storage 940 can be any suitable device that provides storage (e.g., an electrical, magnetic or optical memory including a RAM (volatile and non-volatile), cache, hard drive, or removable storage disk). Communication device 960 can include any suitable device capable of transmitting and receiving signals over a network, such as a network interface chip or device. The components of the computer can be connected in any suitable manner, such as via a wired media (e.g., a physical system bus 980, Ethernet connection, or any other wire transfer technology) or wirelessly (e.g., Bluetooth®, Wi-Fi®, or any other wireless technology).

Software module 950, which can be stored as executable instructions in storage 940 and executed by processor(s) 910, can include, for example, an operating system and/or the processes that embody the functionality of the methods of the present disclosure, e.g., for detecting a RET fusion nucleic acid molecule of the disclosure (e.g., as embodied in the devices as described herein).

Software module 950 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 940, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 950 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

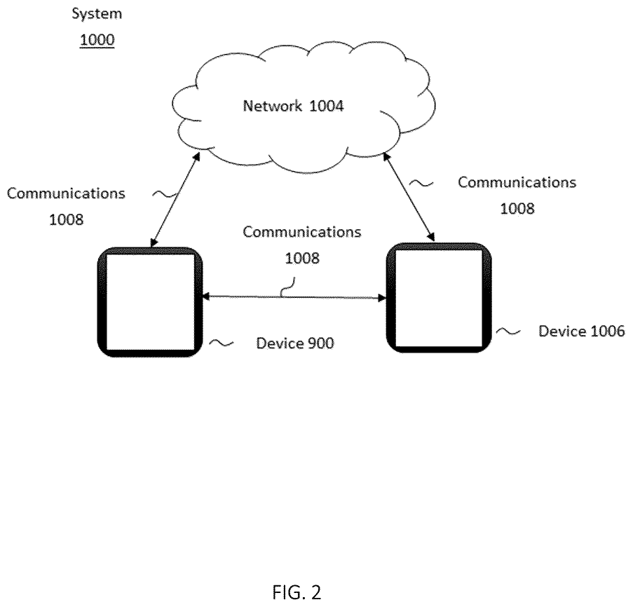

Device 900 may be connected to a network (e.g., network 1004, as shown in FIG. 2 and described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 900 can be implemented using any operating system, e.g., an operating system suitable for operating on the network. Software module 950 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 910.

Device 900 can further include a sequencer 970, which can be any suitable nucleic acid sequencing instrument. Exemplary sequencers can include, without limitation. Roche/454's Genome Sequencer (GS) FLX System. Illumina/Solexa's Genome Analyzer (GA). Illumina's HiSeq 2500, HiSeq 3000, HiSeq 4000 and NovaSeq 6000 Sequencing Systems. Life/APG's Support Oligonucleotide Ligation Detection (SOLID) system. Polonator's G.007 system, Helicos BioSciences” HeliScope Gene Sequencing system, or Pacific Biosciences' PacBio RS system.

FIG. 2 illustrates an example of a computing system in accordance with one embodiment. In computing system 1000, device 900 (e.g., as described above and illustrated in FIG. 1) is connected to network 1004, which is also connected to device 1006. In some embodiments, device 1006 is a sequencer. Exemplary sequencers can include, without limitation. Roche/454's Genome Sequencer (GS) FLX System. Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences PacBio RS system.

Devices 900 and 1006 may communicate, e.g., using suitable communication interfaces via network 1004, such as a Local Area Network (LAN), Virtual Private Network (VPN), or the Internet. In some embodiments, network 1004 can be, for example, the Internet, an intranet, a virtual private network, a cloud network, a wired network, or a wireless network. Devices 900 and 1006 may communicate, in part or in whole, via wireless or hardwired communications, such as Ethernet, IEEE 802.11b wireless, or the like. Additionally, devices 900 and 1006 may communicate, e.g., using suitable communication interfaces, via a second network, such as a mobile/cellular network. Communication between devices 900 and 1006 may further include or communicate with various servers such as a mail server, mobile server, media server, telephone server, and the like. In some embodiments, devices 900 and 1006 can communicate directly (instead of, or in addition to, communicating via network 1004), e.g., via wireless or hardwired communications, such as Ethernet, IEEE 802.11b wireless, or the like. In some embodiments, devices 900 and 1006 communicate via communications 1008, which can be a direct connection or can occur via a network (e.g., network 1004).

One or all of devices 900 and 1006 generally include logic (e.g., http web server logic) or are programmed to format data, accessed from local or remote databases or other sources of data and content, for providing and/or receiving information via network 1004 according to various examples described herein.

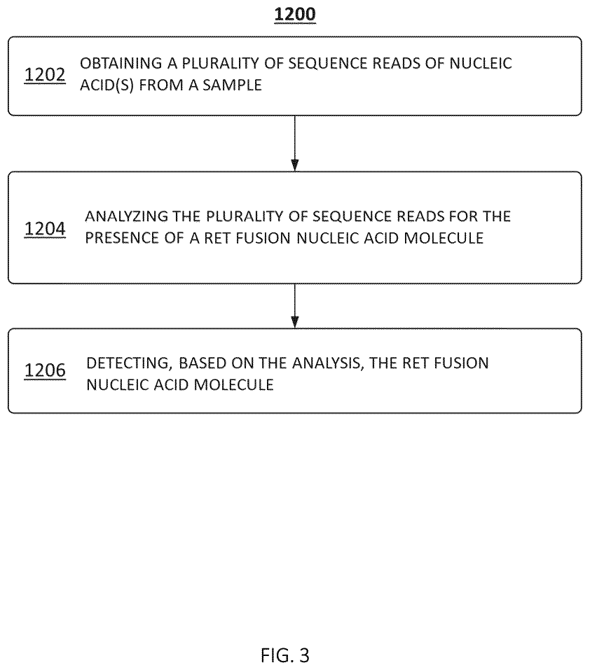

FIG. 3 illustrates an exemplary process 1200 for detecting a RET fusion nucleic acid molecule of the disclosure in a sample, in accordance with some embodiments of the present disclosure. Process 1200 is performed, for example, using one or more electronic devices implementing a software program. In some examples, process 1200 is performed using a client-server system, and the blocks of process 1200 are divided up in any manner between the server and a client device. In other examples, the blocks of process 1200 are divided up between the server and multiple client devices. Thus, while portions of process 1200 are described herein as being performed by particular devices of a client-server system, it will be appreciated that process 1200 is not so limited. In some embodiments, the executed steps can be executed across many systems, e.g., in a cloud environment. In other examples, process 1200 is performed using only a client device or only multiple client devices. In process 1200, some blocks are, optionally, combined, the order of some blocks is, optionally, changed, and some blocks are, optionally, omitted. In some examples, additional steps may be performed in combination with the process 1200. Accordingly, the operations as illustrated (and described in greater detail below) are exemplary by nature and, as such, should not be viewed as limiting.

At block 1202, a plurality of sequence reads of one or more nucleic acid molecules is obtained, wherein the one or more nucleic acid molecules are derived from a sample obtained from an individual, e.g., as described herein. In some embodiments, the sample is obtained from an individual having, suspected of having, or being tested for a cancer, such as a cancer described herein. In some embodiments, the sequence reads are obtained using a sequencer, e.g., as described herein or otherwise known in the art. In some embodiments, the nucleic acid molecules comprise one or more nucleic acid molecules corresponding to: a RET fusion nucleic acid molecule of the disclosure (e.g., any of the RET fusion nucleic acid molecules described above and/or in the Examples herein); or a gene involved in a RET fusion nucleic acid molecule of the disclosure; or fragments thereof. Optionally, prior to obtaining the sequence reads, the sample is purified, enriched (e.g., for nucleic acid(s) corresponding to: a RET fusion nucleic acid molecule of the disclosure; or a gene involved in a RET fusion nucleic acid molecule of the disclosure; or fragments thereof), and/or subjected to PCR amplification. At block 1204, an exemplary system (e.g., one or more electronic devices) analyzes the plurality of sequence reads for the presence of a RET fusion nucleic acid molecule of the disclosure, or a fragment thereof. At block 1206, the system detects (e.g., based on the analysis) a RET fusion nucleic acid molecule of the disclosure, or a fragment thereof, in the sample.

In some embodiments of any of the methods, systems, devices, non-transitory computer readable storage media, or processes of the disclosure, the RET fusion nucleic acid molecule or polypeptide is any of the RET fusion nucleic acid molecules or polypeptides described herein (e.g., as described above, e.g., in Section A, and/or in the Examples herein). In some embodiments of any of the methods, systems, devices, non-transitory computer readable storage media, or processes of the disclosure, the cancer is any cancer known in the art or described herein (e.g., as described above, e.g., in Section A, and/or in the Examples herein). In some embodiments, any of the cancers described herein (e.g., as described above, for example, in Section A herein; and/or in the Examples herein) may comprise any of the RET fusion nucleic acid molecules or polypeptides of the disclosure (e.g., as described above, for example, in Section A herein; and/or in the Examples herein).

In some embodiments of any of the methods, systems, devices, non-transitory computer readable storage media, or processes of the disclosure, detection of a RET fusion nucleic acid molecule or polypeptide of the disclosure, in a cancer (e.g., in one or more samples) identifies the individual having the cancer as one who may benefit from a treatment comprising an anti-cancer therapy, e.g., an anti-cancer therapy provided herein, such as a RET-targeted therapy. In some embodiments of any of the methods, systems, devices, non-transitory computer readable storage media, or processes of the disclosure, detection of a RET fusion nucleic acid molecule or polypeptide of the disclosure in a cancer (e.g., in one or more samples) predicts the individual having the cancer to have longer survival when treated with a treatment comprising an anti-cancer therapy, e.g., a RET-targeted therapy, as compared to survival of an individual whose cancer does not comprise a RET fusion nucleic acid molecule or polypeptide. In some embodiments of any of the methods, systems, devices, non-transitory computer readable storage media, or processes of the disclosure, detection of a RET fusion nucleic acid molecule or polypeptide of the disclosure in a cancer (e.g., in one or more samples) identifies the individual having the cancer to be a candidate to receive a treatment comprising an anti-cancer therapy, e.g., a RET-targeted therapy. In some embodiments of any of the methods, systems, devices, non-transitory computer readable storage media, or processes of the disclosure, detection of a RET fusion nucleic acid molecule or polypeptide of the disclosure in a cancer (e.g., in one or more samples) identifies the individual having the cancer as likely to respond (e.g., to have a therapeutic response) to a treatment comprising an anti-cancer therapy, e.g., a RET-targeted therapy. In some embodiments of any of the methods, systems, devices, non-transitory computer readable storage media, or processes of the disclosure, detection of a RET fusion nucleic acid molecule or polypeptide of the disclosure in a cancer (e.g., in one or more samples) identifies the individual having the cancer as likely to have an improved response when treated with a treatment comprising an anti-cancer therapy, e.g., a RET-targeted therapy, as compared to an individual whose cancer does not comprise a RET fusion nucleic acid molecule or polypeptide.

In some embodiments of any of the methods, systems, devices, non-transitory computer readable storage media, or processes of the disclosure, the plurality of sequence reads is obtained by sequencing nucleic acids obtained from any of the samples described herein, e.g., tissue and/or liquid biopsies, etc. In some embodiments, the sample is obtained from the cancer. In some embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, cerebrospinal fluid, sputum, stool, urine, or saliva. In some embodiments, the sample comprises cells and/or nucleic acids from the cancer. In some embodiments, the sample comprises mRNA, DNA, circulating tumor DNA (ctDNA), cell-free DNA, or cell-free RNA from the cancer. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof.

In some embodiments of any of the methods, systems, devices, non-transitory computer readable storage media, or processes of the disclosure, the plurality of sequence reads is obtained by sequencing. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the massively parallel sequencing technique comprises next generation sequencing (NGS).

In some embodiments of any of the methods, systems, devices, non-transitory computer readable storage media, or processes of the disclosure, the individual is administered a treatment based at least in part on detection of a RET fusion nucleic acid molecule or polypeptide of the disclosure in the cancer in an individual (e.g., in one or more samples from the individual). In some embodiments, the treatment is an anti-cancer therapy known in the art or described herein, e.g., a RET-targeted therapy.

In some embodiments of any of the methods, systems, devices, non-transitory computer readable storage media, or processes of the disclosure, the disclosed methods for determining the presence or absence of a RET fusion nucleic acid molecule of the disclosure may be implemented as part of a genomic profiling process that comprises identification of the presence of variant sequences at one or more gene loci in a sample derived from an individual as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), a next-generation sequencing (NGS) approach used to assess hundreds of genes (including relevant cancer biomarkers) in a single assay. Inclusion of the disclosed methods for determining the presence or absence of a RET fusion nucleic acid molecule of the disclosure as part of a genomic profiling process can improve the validity of, e.g., disease detection calls, made on the basis of the genomic profiling by, for example, independently confirming the presence of the RET fusion nucleic acid molecule of the disclosure in a given patient sample. In some instances, the comprehensive genomic profiling may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors. In some instances, the comprehensive genomic profiling may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof. In some embodiments of any of the methods, systems, devices, non-transitory computer readable storage media, or processes of the disclosure, a molecular profile for a sample or for an individual is generated based at least in part on detecting a RET fusion nucleic acid molecule of the disclosure, or a fragment thereof, in a sample. In some instances, the molecular profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors. In some instances, the molecular profile may comprise results from a comprehensive genomic profiling (CGP) test (e.g., as describe above), a nucleic acid sequencing-based test, a gene expression profiling test, a cancer hotspot panel test, a DNA methylation test, a DNA fragmentation test, an RNA fragmentation test, or any combination thereof. In some embodiments, the molecular profile further comprises/indicates/comprises information on presence or absence of mutations in one or more additional genes, e.g., a panel of known/suspected oncogenes and/or tumor suppressors. In some embodiments, the molecular profile is obtained from a genomic profiling assay (such as a cancer- or tumor-related genomic profiling assay), e.g., as obtained using any of the sequencing methodologies described herein. In some embodiments, the molecular profile includes information from whole-genome or whole-exome sequencing. In some embodiments, the molecular profile includes information from targeted sequencing. In some embodiments, the molecular profile includes information from NGS. In some embodiments, the molecular profile comprises/indicates/comprises information on presence or absence of mutations such as short variant alterations (e.g., a base substitution, insertion, or deletion), copy-number alterations (e.g., an amplification or a homozygous deletion), and/or rearrangements (e.g., a gene fusion or other genomic or chromosomal rearrangement) of one or more genes, e.g., a panel of known/suspected oncogenes and/or tumor suppressors, one or more cancer-related genes, or any combination thereof. In some embodiments, the individual is administered a treatment based at least in part on the molecular profile. In some embodiments, the treatment is an anti-cancer therapy known in the art or described herein, e.g., a RET-targeted therapy. In some embodiments, the one or more genes or gene loci comprise one or more known/suspected oncogenes and/or tumor suppressors, one or more cancer-related genes, or any combination thereof. Alternatively or additionally, in some embodiments, the one or more genes or gene loci comprise one or more of ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKNIA, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNFIA, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MSTIR, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2RIA, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSCIL1, WT1, XPO1, XRCC2, ZNF217, ZNF703, or any combination thereof. Alternatively or additionally, in some embodiments, the one or more genes or gene loci comprise one or more of ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1B, IL-6, IL-6R, JAK1, JAK2, JAK3, KIT, KRAS, MEK, MET, MSI-H, mTOR, PARP, PD-1, PDGFR, PDGFRa, PDGFRB, PD-L1, PI3K8, PIGF, PTCH, RAF, RANKL, RET, ROS1, SLAMF7, VEGF, VEGFA, VEGFB, or any combination thereof. Alternatively or additionally, in some embodiments, the one or more genes or gene loci comprise one or more of CPEB3, OPTN, GRM7, AGBL4, PPP2R5A, OXR1, LRMDA, MGEA5, ADCY1, SAMD4A, HSD17B7P2, ACPP, ZNF248, MKX, MYH14, BMS1, GOLGB1, NTRK2, ABI3BP, ARHGAP19, GP2, SH2D3A, VSTM4, RBMS3, LINC00379, ZNF721, ZSWIM6, SGIP1, DCC, CCNY, WDFY4, EEA1, ELMO1, RAD1, NAALADL2, CCBE1, CEP135, RAI14, ADAMTS14, SORBS1, CSGALNACT2, PCM1, KIAA1217, MPRIP, TFG, SPECC1L, REEP3, RRBP1, ETV6, TAF3, RASGEF1A, TNIP2, SATB1, ALOX5, ANKRD26, CLIP1, DLG5, ERC1, FRMD4A, KIAA1468, NCOA4, PARD3, PRKAR1A, PRKG1, RUFY2, SNRNP70, SQSTM1, TNIP1, TRIM27, TRIM33, CCDC6, TRIM24, FGFR1OP, GAS2, HOOK1, LMNA, MLPH, MYH9, PCDH15, PIBF1, SLC12A2, or ZNF485, or any gene listed in Tables 1-2, and any combination thereof.

In some embodiments of any of the methods, systems, devices, non-transitory computer readable storage media, or processes of the disclosure, a report is generated, e.g., as described in further detail above. In some embodiments, the report comprises/indicates/comprises information on the presence or absence of a RET fusion nucleic acid molecule or polypeptide of the disclosure in the cancer in an individual (e.g., in one or more samples from the individual). In some embodiments, the report comprises/indicates/comprises information on results of a genomic profiling process of a cancer in an individual (e.g., in one or more samples from the individual), e.g., as described above. In some embodiments, the report comprises/indicates/comprises information on results of comprehensive genomic profiling of a cancer in an individual (e.g., in one or more samples from the individual), e.g., as described above. In some embodiments, the report comprises/indicates/comprises information on a molecular profile generated for the individual or the sample, e.g., as described above. In some embodiments, the report comprises/indicates/comprises information on a treatment or one or more treatment options selected or identified for the individual, based, at least in part, on the presence of a RET fusion nucleic acid molecule or polypeptide of the disclosure in the cancer in an individual (e.g., in one or more samples from the individual), and optionally based on results of a genomic profiling process, comprehensive genomic profiling, and/or a molecular profile generated for the individual or a sample, e.g., as described above. In some embodiments, the treatment or one or more treatment options comprise an anti-cancer therapy known in the art or described herein, e.g., a RET-targeted therapy. In some embodiments, the report is provided or transmitted to the individual, a caregiver, a healthcare provider, a physician, an oncologist, an electronic medical record system, a hospital, a clinic, a third-party payer, an insurance company, or a government office, e.g., as described in further detail above. In some embodiments, the report is transmitted via a computer network or a peer-to-peer connection. In some embodiments, an individual is administered a treatment based, at least in part, on the report. In some instances, all or a portion of the report may be displayed in a graphical user interface of an online or web-based healthcare portal.

The method steps of the methods described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

### IV. Articles of Manufacture or Kits

Provided herein are kits or articles of manufacture comprising one or more reagents for detecting a RET fusion nucleic acid molecule or a RET fusion polypeptide of the disclosure (e.g., any of the RET fusion nucleic acid molecules or RET fusion polypeptides described above and/or in the Examples herein) in a sample.

In some embodiments, the kits or articles of manufacture comprise one or more probes of the disclosure for detecting a RET fusion nucleic acid molecule of the disclosure in a sample, e.g., according to any detection method known in the art or described herein. In some embodiments, the kits or articles of manufacture comprise one or more baits (e.g., one or more bait molecules) of the disclosure for detecting a RET fusion nucleic acid molecule of the disclosure in a sample, e.g., according to any detection method known in the art or described herein. In some embodiments, the kits or articles of manufacture comprise one or more oligonucleotides (e.g., one or more primers) of the disclosure for detecting a RET fusion nucleic acid molecule of the disclosure in a sample, e.g., according to any detection method known in the art or described herein. In some embodiments of any of the kits or articles of manufacture provided herein, the kit or article of manufacture comprises a reagent (e.g., one or more oligonucleotides, primers, probes or baits of the present disclosure) for detecting a wild-type counterpart of a RET fusion nucleic acid molecule of the disclosure (e.g., a wild type RET gene, and/or a wild type fusion partner gene described herein and/or in Tables 1-10, and/or in the Examples herein). In some embodiments, one or more oligonucleotides, primers, probes or baits are capable of hybridizing to a RET fusion nucleic acid molecule of the disclosure, or to a wild-type counterpart of the RET fusion nucleic acid molecule (e.g., a wild type RET gene, and/or a wild type fusion partner gene described herein and/or in Tables 1-10, and/or in the Examples herein). In some embodiments, the one or more oligonucleotides, primers, probes or baits of the present disclosure are capable of distinguishing a RET fusion nucleic acid molecule of the disclosure from a wild-type counterpart of the RET fusion nucleic acid molecule (e.g., a wild type RET gene, and/or a wild type fusion partner gene described herein and/or in Tables 1-10, and/or in the Examples herein). In some embodiments, the kit is for use according to any method of detecting fusion nucleic acid molecules known in the art or described herein, such as sequencing, PCR, in situ hybridization methods, a nucleic acid hybridization assay, an amplification-based assay, a PCR-RFLP assay, real-time PCR, sequencing, next-generation sequencing, a screening analysis, FISH, spectral karyotyping, MFISH, comparative genomic hybridization, in situ hybridization, sequence-specific priming (SSP) PCR. HPLC, and mass-spectrometric genotyping. In some embodiments, a kit provided herein further comprises instructions for detecting a RET fusion nucleic acid molecule of the disclosure, e.g., using one or more oligonucleotides, primers, probes or baits of the present disclosure.

In some embodiments, the kits or articles of manufacture comprise one or more antibodies or antibody fragments of the disclosure for detecting a RET fusion polypeptide of the disclosure, e.g., according to any detection method known in the art or described herein. In some embodiments, the kit or article of manufacture comprises a reagent (e.g., one or more antibodies of the present disclosure) for detecting the wild-type counterparts of a RET fusion polypeptide provided herein (e.g., a wild type RET polypeptide, and/or a wild type polypeptide encoded by a fusion partner gene described herein and/or in Tables 1-10, and/or in the Examples herein). In some embodiments, the kits or articles of manufacture comprise one or more antibodies of the present disclosure capable of binding to a RET fusion polypeptide provided herein, or to wild-type counterparts of the RET fusion polypeptide provided herein (e.g., a wild type RET polypeptide, and/or a wild type polypeptide encoded by a fusion partner gene described herein and/or in Tables 1-10, and/or in the Examples herein). In some embodiments, the kit is for use according to any protein or polypeptide detection assay known in the art or described herein, such as mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography). In some embodiments, the kit further comprises instructions for detecting a RET fusion polypeptide of the disclosure, e.g., using one or more antibodies of the present disclosure.

Further provided herein are kits or articles of manufacture comprising an anti-cancer therapy, such as an anti-cancer therapy described herein, and a package insert comprising instructions for using the anti-cancer therapy in a method of treating or delaying progression of cancer, e.g., by administration to an individual from whom a sample comprising a RET fusion nucleic acid molecule or a RET fusion polypeptide of the disclosure has been obtained. In some embodiments, the anti-cancer therapy is any of the anti-cancer therapies described herein for use in any of the methods for treating or delaying progression of cancer of the disclosure. In some embodiments, the anti-cancer therapy is or comprises a RET-targeted therapy.

The kit or article of manufacture may include, for example, a container and a label or package insert on or associated with the container. Suitable containers include, for example, bottles, vials, syringes, and the like. The container may be formed from a variety of materials such as glass or plastic. The container holds or contains a composition comprising one or more reagents for detecting a RET fusion nucleic acid molecule or polypeptide of the disclosure (e.g., one or more oligonucleotides, primers, probes, baits, antibodies or antibody fragments of the present disclosure) or one or more anti-cancer therapies of the disclosure. In some embodiments, the container holds or contains a composition comprising one or more anti-cancer therapies of the disclosure and may have a sterile access port (e.g., the container may be an intravenous solution bag or a vial having a stopper pierceable by a hypodermic injection needle).

The kit or article of manufacture may further include a second container comprising a diluent or buffer, e.g., a pharmaceutically-acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

The kit or article of manufacture of the present disclosure also includes information or instructions, for example in the form of a package insert, indicating that the one or more reagents and/or anti-cancer therapies are used for detecting a RET fusion nucleic acid molecule or a RET fusion polypeptide of the disclosure, or for treating cancer, as described herein. The insert or label may take any form, such as paper or on electronic media such as a magnetically recorded medium (e.g., floppy disk), a CD-ROM, a Universal Serial Bus (USB) flash drive, and the like. The label or insert may also include other information concerning the pharmaceutical compositions and dosage forms in the kit or article of manufacture.

### V. Nucleic Acids, Vectors, Host Cells and Recombinant Cells

Provided herein are nucleic acids and vectors comprising or encoding a RET fusion nucleic acid molecule of the disclosure (e.g., any of the RET fusion nucleic acid molecules described above and/or in the Examples herein), or a bait, a probe, or an oligonucleotide described herein, or fragments thereof.

In some embodiments, a nucleic acid or vector provided herein comprises or encodes a RET fusion nucleic acid molecule of the disclosure, or a nucleic acid molecule encoding a RET fusion polypeptide described herein.

In some embodiments, a vector provided herein is a nucleic acid molecule capable of transporting another nucleic acid to which it has been linked (e.g., RET fusion nucleic acid molecules, baits, probes, or oligonucleotides described herein, or fragments thereof). In some embodiments, a vector is a plasmid, a cosmid or a viral vector. The vector may be capable of autonomous replication, or it can integrate into a host DNA. Viral vectors (e.g., comprising RET fusion nucleic acid molecules, baits, probes, or oligonucleotides described herein, or fragments thereof) are also contemplated herein, including, e.g., replication defective retroviruses, adenoviruses and adeno-associated viruses.

In some embodiments, a nucleic acid or vector provided herein comprises a RET fusion nucleic acid molecule, a bait, a probe, or an oligonucleotide of the disclosure in a form suitable for expression thereof in a host cell. In some embodiments, the nucleic acid or vector includes one or more regulatory sequences operatively linked to the nucleotide sequence to be expressed. In some embodiments, the one or more regulatory sequences include promoters (e.g., promoters derived from polyoma, Adenovirus 2, cytomegalovirus and Simian Virus 40), enhancers, and other expression control elements (e.g., polyadenylation signals). In some embodiments, a regulatory sequence directs constitutive expression of a nucleotide sequence (e.g., RET fusion nucleic acid molecules, baits, probes, or oligonucleotides described herein, or fragments thereof). In some embodiments, a regulatory sequence directs tissue-specific expression of a nucleotide sequence (e.g., RET fusion nucleic acid molecules, baits, probes, or oligonucleotides described herein, or fragments thereof). In some embodiments, a regulatory sequence directs inducible expression of a nucleotide sequence (e.g., RET fusion nucleic acid molecules, baits, probes, or oligonucleotides described herein, or fragments thereof). Examples of inducible regulatory sequences include, without limitation, promoters regulated by a steroid hormone, by a polypeptide hormone, or by a heterologous polypeptide, such as a tetracycline-inducible promoter. Examples of tissue- or cell-type-specific regulatory sequences include, without limitation, the albumin promoter, lymphoid-specific promoters, promoters of T cell receptors or immunoglobulins, neuron-specific promoters, pancreas-specific promoters, mammary gland-specific promoters, developmentally-regulated promoters, and the like. In some embodiments, a vector provided herein comprises or encodes a RET fusion nucleic acid molecule, a bait, a probe, or an oligonucleotide of the disclosure in the sense or the anti-sense orientation. In some embodiments, a nucleic acid or vector (e.g., an expression vector) provided herein is introduced into host cells to thereby produce a polypeptide, e.g., a RET fusion polypeptide described herein, or a fragment or mutant form thereof.

In some embodiments, the design of a nucleic acid or vector provided herein depends on such factors as the choice of the host cell to be transformed, the level of expression desired, and the like. In some embodiments, expression vectors are designed for the expression of the RET fusion nucleic acid molecules, baits, probes, or oligonucleotides described herein, or fragments thereof, in prokaryotic or eukaryotic cells, such as E, coli cells, insect cells (e.g., using baculovirus expression vectors), yeast cells, or mammalian cells. In some embodiments, a vector described herein is transcribed and translated in vitro, for example using T7 promoter regulatory sequences and T7 polymerase. In some embodiments, a vector (e.g., an expression vector) provided herein comprises or encodes a RET fusion nucleic acid molecule described herein, wherein the nucleotide sequence of the RET fusion nucleic acid molecule described herein has been altered (e.g., codon optimized) so that the individual codons for each encoded amino acid are those preferentially utilized in the host cell.

Also provided herein are host cells, e.g., comprising RET fusion nucleic acid molecules, RET fusion polypeptides, baits, probes, nucleic acids, vectors, or oligonucleotides of the disclosure. In some embodiments, a host cell (e.g., a recombinant host cell or recombinant cell) comprises a vector described herein (e.g., an expression vector described herein). In some embodiments, a RET fusion nucleic acid molecule, bait, probe, nucleic acid, vector, or oligonucleotide provided herein further includes sequences which allow it to integrate into the host cell's genome (e.g., through homologous recombination at a specific site). In some embodiments, a host cell provided herein is a prokaryotic or eukaryotic cell. Non limiting examples of host cells include, without limitation, bacterial cells (e.g., E. coli), insect cells, yeast cells, or mammalian cells (e.g., human cells, rodent cells, mouse cells, rabbit cells, pig cells, Chinese hamster ovary cells (CHO), or COS cells, e.g., COS-7 cells, CV-1 origin SV40 cells). A host cell described herein includes the particular host cell, as well as the progeny of such a cell. Because certain modifications may occur in succeeding generations due to either mutation or environmental influences, such progeny may not, in fact, be identical to the parent host cell.

RET fusion nucleic acid molecules, baits, probes, nucleic acids, vectors, or oligonucleotides of the disclosure may be introduced into host cells using any suitable method known in the art, such as conventional transformation or transfection techniques (e.g., using calcium phosphate or calcium chloride co-precipitation, DEAE-dextran-mediated transfection, lipofection, or electroporation).

Also provided herein are methods of producing a RET fusion polypeptide of the disclosure, e.g., by culturing a host cell described herein (e.g., into which a recombinant expression vector encoding a RET fusion polypeptide has been introduced) in a suitable medium such that the RET fusion polypeptide is produced. In another embodiment, the method further includes isolating a RET fusion polypeptide from the medium or the host cell.

### VI. Exemplary Embodiments

The following exemplary embodiments are representative of some aspects of the invention.

Exemplary Embodiment 1: A method of identifying an individual having a cancer who may benefit from a treatment comprising a rearranged during transfection (RET)-targeted therapy, the method comprising detecting in a sample from the individual a RET fusion nucleic acid molecule, or a RET fusion polypeptide encoded by the RET fusion nucleic acid molecule, wherein:
(a) the RET fusion nucleic acid molecule comprises a fusion between a RET gene, or a portion thereof, and a gene listed in Table 1, or a portion thereof, or
(b) the RET fusion nucleic acid molecule comprises a fusion between a RET gene, or a portion thereof, and a gene listed in Table 2, or a portion thereof, and the cancer is the cancer corresponding to the gene listed in Table 2,
and wherein detection of the RET fusion nucleic acid molecule, or the RET fusion polypeptide encoded by the RET fusion nucleic acid molecule, in the sample identifies the individual as one who may benefit from the treatment comprising the RET-targeted therapy.

Exemplary Embodiment 2: A method of selecting a therapy for an individual having a cancer, the method comprising detecting in a sample from the individual a RET fusion nucleic acid molecule, or a RET fusion polypeptide encoded by the RET fusion nucleic acid molecule, wherein:
(a) the RET fusion nucleic acid molecule comprises a fusion between a RET gene, or a portion thereof, and a gene listed in Table 1, or a portion thereof, or
(b) the RET fusion nucleic acid molecule comprises a fusion between a RET gene, or a portion thereof, and a gene listed in Table 2, or a portion thereof, and the cancer is the cancer corresponding to the gene listed in Table 2,
and wherein detection of the RET fusion nucleic acid molecule, or the RET fusion polypeptide encoded by the RET fusion nucleic acid molecule, in the sample identifies the individual as one who may benefit from a treatment comprising a RET-targeted therapy.

Exemplary Embodiment 3: A method of identifying one or more treatment options for an individual having a cancer, the method comprising:
(a) detecting in a sample from the individual a RET fusion nucleic acid molecule, or a RET fusion polypeptide encoded by the RET fusion nucleic acid molecule, wherein:
   (i) the RET fusion nucleic acid molecule comprises a fusion between a RET gene, or a portion thereof, and a gene listed in Table 1, or a portion thereof, or
   (ii) the RET fusion nucleic acid molecule comprises a fusion between a RET gene, or a portion thereof, and a gene listed in Table 2, or a portion thereof, and the cancer is the cancer corresponding to the gene listed in Table 2; and
(b) generating a report comprising one or more treatment options identified for the individual based at least in part on detection of the RET fusion nucleic acid molecule, or the RET fusion polypeptide encoded by the RET fusion nucleic acid molecule, in the sample, wherein the one or more treatment options comprise a RET-targeted therapy.

Exemplary Embodiment 4: A method of identifying one or more treatment options for an individual having a cancer, the method comprising:
(a) acquiring knowledge of a RET fusion nucleic acid molecule, or a RET fusion polypeptide encoded by the RET fusion nucleic acid molecule, in a sample from the individual, wherein:
   (i) the RET fusion nucleic acid molecule comprises a fusion between a RET gene, or a portion thereof, and a gene listed in Table 1, or a portion thereof, or
   (ii) the RET fusion nucleic acid molecule comprises a fusion between a RET gene, or a portion thereof, and a gene listed in Table 2, or a portion thereof, and the cancer is the cancer corresponding to the gene listed in Table 2; and
(b) generating a report comprising one or more treatment options identified for the individual based at least in part on said knowledge, wherein the one or more treatment options comprise a RET-targeted therapy.

Exemplary Embodiment 5: A method of selecting a treatment for an individual having cancer, comprising acquiring knowledge of a RET fusion nucleic acid molecule, or a RET fusion polypeptide encoded by the RET fusion nucleic acid molecule, in a sample from the individual, wherein:
(a) the RET fusion nucleic acid molecule comprises a fusion between a RET gene, or a portion thereof, and a gene listed in Table 1, or a portion thereof, or
(b) the RET fusion nucleic acid molecule comprises a fusion between a RET gene, or a portion thereof, and a gene listed in Table 2, or a portion thereof, and the cancer is the cancer corresponding to the gene listed in Table 2,
and wherein responsive to the acquisition of said knowledge: (i) the individual is classified as a candidate to receive a treatment comprising a RET-targeted therapy; and/or (ii) the individual is identified as likely to respond to a treatment that comprises a RET-targeted therapy.

Exemplary Embodiment 6: A method of predicting survival of an individual having a cancer, comprising acquiring knowledge of a RET fusion nucleic acid molecule, or a RET fusion polypeptide encoded by the RET fusion nucleic acid molecule, in a sample from the individual, wherein:
(a) the RET fusion nucleic acid molecule comprises a fusion between a RET gene, or a portion thereof, and a gene listed in Table 1, or a portion thereof, or
(b) the RET fusion nucleic acid molecule comprises a fusion between a RET gene, or a portion thereof, and a gene listed in Table 2, or a portion thereof, and the cancer is the cancer corresponding to the gene listed in Table 2,
and wherein responsive to the acquisition of said knowledge, the individual is predicted to have longer survival when treated with a treatment comprising a RET-targeted therapy, as compared to survival of an individual whose cancer does not comprise a RET fusion nucleic acid molecule or a RET fusion polypeptide.

Exemplary Embodiment 7: A method of predicting survival of an individual having a cancer treated with a treatment comprising a RET-targeted therapy, the method comprising acquiring knowledge of a RET fusion nucleic acid molecule, or a RET fusion polypeptide encoded by the RET fusion nucleic acid molecule, in a sample from the individual, wherein:
(a) the RET fusion nucleic acid molecule comprises a fusion between a RET gene, or a portion thereof, and a gene listed in Table 1, or a portion thereof, or
(b) the RET fusion nucleic acid molecule comprises a fusion between a RET gene, or a portion thereof, and a gene listed in Table 2, or a portion thereof, and the cancer is the cancer corresponding to the gene listed in Table 2, and
wherein responsive to the acquisition of said knowledge, the individual is predicted to have longer survival when treated with a treatment comprising a RET-targeted therapy, as compared to an individual whose cancer does not exhibit a RET fusion nucleic acid molecule or a RET fusion polypeptide.

Exemplary Embodiment 8: A method of treating or delaying progression of cancer in an individual, comprising:
(a) acquiring knowledge of a RET fusion nucleic acid molecule, or a RET fusion polypeptide encoded by the RET fusion nucleic acid molecule, in a sample from the individual, wherein:
   (i) the RET fusion nucleic acid molecule comprises a fusion between a RET gene, or a portion thereof, and a gene listed in Table 1, or a portion thereof, or
   (ii) the RET fusion nucleic acid molecule comprises a fusion between a RET gene, or a portion thereof, and a gene listed in Table 2, or a portion thereof, and the cancer is the cancer corresponding to the gene listed in Table 2; and
(b) responsive to said knowledge, administering to the individual an effective amount of a treatment that comprises a RET-targeted therapy.

Exemplary Embodiment 9: A method of treating or delaying progression of cancer, comprising administering to an individual having cancer an effective amount of a treatment that comprises a RET-targeted therapy, wherein the RET-targeted therapy is administered responsive to acquiring knowledge of a RET fusion nucleic acid molecule, or a RET fusion polypeptide encoded by the RET fusion nucleic acid molecule, in a sample from the individual, wherein:
(a) the RET fusion nucleic acid molecule comprises a fusion between a RET gene, or a portion thereof, and a gene listed in Table 1, or a portion thereof, or
(b) the RET fusion nucleic acid molecule comprises a fusion between a RET gene, or a portion thereof, and a gene listed in Table 2, or a portion thereof, and the cancer is the cancer corresponding to the gene listed in Table 2.

Exemplary Embodiment 10: A method of monitoring, evaluating or screening an individual having a cancer, comprising acquiring knowledge of a RET fusion nucleic acid molecule, or a RET fusion polypeptide encoded by the RET fusion nucleic acid molecule, in a sample from the individual, wherein:
(a) the RET fusion nucleic acid molecule comprises a fusion between a RET gene, or a portion thereof, and a gene listed in Table 1, or a portion thereof, or
(b) the RET fusion nucleic acid molecule comprises a fusion between a RET gene, or a portion thereof, and a gene listed in Table 2, or a portion thereof, and the cancer is the cancer corresponding to the gene listed in Table 2,
and wherein responsive to the acquisition of said knowledge, the individual is predicted to have increased risk of cancer recurrence, aggressive cancer, anti-cancer therapy resistance, increased RET expression, clinical benefit from a RET-targeted therapy, or poor prognosis, as compared to an individual whose cancer does not comprise a RET fusion nucleic acid molecule or a RET fusion polypeptide.

Exemplary Embodiment 11: The method of embodiment 10, wherein responsive to the acquisition of said knowledge, the individual is predicted to have resistance to a non-RET-targeted anti-cancer therapy.

Exemplary Embodiment 12: A method of assessing a RET fusion nucleic acid molecule or a RET fusion polypeptide in a cancer in an individual, the method comprising:
(a) detecting a RET fusion nucleic acid molecule, or a RET fusion polypeptide encoded by the RET fusion nucleic acid molecule, in a sample from the individual, wherein:
   (i) the RET fusion nucleic acid molecule comprises a fusion between a RET gene, or a portion thereof, and a gene listed in Table 1, or a portion thereof, or
   (ii) the RET fusion nucleic acid molecule comprises a fusion between a RET gene, or a portion thereof, and a gene listed in Table 2, or a portion thereof, and the cancer is the cancer corresponding to the gene listed in Table 2; and
(b) providing an assessment of the RET fusion nucleic acid molecule, or the RET fusion polypeptide encoded by the RET fusion nucleic acid molecule, in the sample.

Exemplary Embodiment 13: A method of detecting a RET fusion nucleic acid molecule or a RET fusion polypeptide, the method comprising detecting in a sample from an individual having a cancer a RET fusion nucleic acid molecule, or a RET fusion polypeptide encoded by the RET fusion nucleic acid molecule, wherein:
(a) the RET fusion nucleic acid molecule comprises a fusion between a RET gene, or a portion thereof, and a gene listed in Table 1, or a portion thereof, or
(b) the RET fusion nucleic acid molecule comprises a fusion between a RET gene, or a portion thereof, and a gene listed in Table 2, or a portion thereof, and the cancer is the cancer corresponding to the gene listed in Table 2.

Exemplary Embodiment 14: A method of detecting the presence or absence of a cancer in an individual, the method comprising:
(a) detecting the presence or absence of a cancer in a sample from the individual; and
(b) detecting the presence or absence of a RET fusion nucleic acid molecule, or a RET fusion polypeptide encoded by the RET fusion nucleic acid molecule, in a sample from the individual, wherein:
   (i) the RET fusion nucleic acid molecule comprises a fusion between a RET gene, or a portion thereof, and a gene listed in Table 1, or a portion thereof, or
   (ii) the RET fusion nucleic acid molecule comprises a fusion between a RET gene, or a portion thereof, and a gene listed in Table 2, or a portion thereof, and the cancer is the cancer corresponding to the gene listed in Table 2.

Exemplary Embodiment 15: The method of embodiment 14, comprising detecting the presence of the cancer in a sample from the individual.

Exemplary Embodiment 16: The method of embodiment 14 or embodiment 15, comprising detecting the presence of the RET fusion nucleic acid molecule, or the RET fusion polypeptide encoded by the RET fusion nucleic acid molecule, in a sample from the individual.

Exemplary Embodiment 17: A method for monitoring progression or recurrence of a cancer in an individual, the method comprising:
(a) detecting, in a first sample obtained from the individual at a first time point, the presence or absence of a RET fusion nucleic acid molecule, or a RET fusion polypeptide encoded by the RET fusion nucleic acid molecule;
(b) detecting, in a second sample obtained from the individual at a second time point after the first time point, the presence or absence of a RET fusion nucleic acid molecule, or a RET fusion polypeptide encoded by the RET fusion nucleic acid molecule; and
(c) providing an assessment of cancer progression or cancer recurrence in the individual based, at least in part, on the presence or absence of the RET fusion nucleic acid molecule, or the RET fusion polypeptide encoded by the RET fusion nucleic acid, in the first sample and/or in the second sample, wherein:
   (i) the RET fusion nucleic acid molecule comprises a fusion between a RET gene, or a portion thereof, and a gene listed in Table 1, or a portion thereof, or
   (ii) the RET fusion nucleic acid molecule comprises a fusion between a RET gene, or a portion thereof, and a gene listed in Table 2, or a portion thereof, and the cancer is the cancer corresponding to the gene listed in Table 2.

Exemplary Embodiment 18: The method of embodiment 17, wherein the presence of the RET fusion nucleic acid molecule, or the RET fusion polypeptide encoded by the RET fusion nucleic acid molecule, in the first sample and/or in the second sample identifies the individual as having increased risk of cancer progression or cancer recurrence.

Exemplary Embodiment 19: The method of embodiment 17 or embodiment 18, further comprising selecting a treatment, administering a treatment, adjusting a treatment, adjusting a dose of a treatment, or applying a treatment to the individual based, at least in part, on detecting the presence of the RET fusion nucleic acid molecule, or the RET fusion polypeptide encoded by the RET fusion nucleic acid molecule, in the first sample and/or in the second sample, wherein the treatment comprises a RET-targeted therapy.

Exemplary Embodiment 20: A method of detecting a RET fusion nucleic acid molecule, the method comprising:
(a) providing a plurality of nucleic acid molecules obtained from a sample from an individual having a cancer, wherein the plurality of nucleic acid molecules comprises nucleic acid molecules corresponding to:
   (i) a RET fusion nucleic acid molecule comprising a fusion between a RET gene, or a portion thereof, and a gene listed in Table 1, or a portion thereof, or
   (ii) a RET fusion nucleic acid molecule comprising a fusion between a RET gene, or a portion thereof, and a gene listed in Table 2, or a portion thereof, and the cancer is the cancer corresponding to the gene listed in Table 2;
(b) optionally, ligating one or more adapters onto one or more nucleic acid molecules from the plurality of nucleic acid molecules;
(c) optionally, amplifying the one or more ligated nucleic acid molecules from the plurality of nucleic acid molecules;
(d) optionally, capturing amplified nucleic acid molecules from the amplified nucleic acid molecules;
(e) sequencing, by a sequencer, the captured nucleic acid molecules to obtain a plurality of sequence reads that represent the captured nucleic acid molecules, wherein one or more of the plurality of sequence reads correspond to the RET fusion nucleic acid molecule;
(f) analyzing the plurality of sequence reads; and
(g) based on the analysis, detecting the presence or absence of the RET fusion nucleic acid molecule in the sample.

Exemplary Embodiment 21: The method of embodiment 20, further comprising receiving, at one or more processors, sequence read data for the plurality of sequence reads.

Exemplary Embodiment 22: The method of embodiment 21, wherein the analyzing the plurality of sequence reads comprises identifying, using the one or more processors, the presence or absence of sequence reads corresponding to the RET fusion nucleic acid molecule.

Exemplary Embodiment 23: The method of any one of embodiments 20-22, wherein the amplified nucleic acid molecules are captured by hybridization with one or more bait molecules.

Exemplary Embodiment 24: A method of detecting a RET fusion nucleic acid molecule, the method comprising:
(a) providing a sample from an individual having a cancer, wherein the sample comprises a plurality of nucleic acid molecules;
(b) preparing a nucleic acid sequencing library from the plurality of nucleic acid molecules in the sample;
(c) amplifying said library;
(d) selectively enriching for one or more nucleic acid molecules comprising nucleotide sequences corresponding to a RET fusion nucleic acid molecule in said library to produce an enriched sample, wherein:
   (i) the RET fusion nucleic acid molecule comprises a fusion between a RET gene, or a portion thereof, and a gene listed in Table 1, or a portion thereof, or
   (ii) the RET fusion nucleic acid molecule comprises a fusion between a RET gene, or a portion thereof, and a gene listed in Table 2, or a portion thereof, and the cancer is the cancer corresponding to the gene listed in Table 2;
(e) sequencing the enriched sample, thereby producing a plurality of sequence reads;
(f) analyzing the plurality of sequence reads for the presence of the RET fusion nucleic acid molecule; and
(g) detecting, based on the analyzing step, the presence or absence of the RET fusion nucleic acid molecule in the sample from the individual.

Exemplary Embodiment 25: The method of any one of embodiments 20-24, wherein the plurality of nucleic acid molecules comprises a mixture of cancer nucleic acid molecules and non-cancer nucleic acid molecules.

Exemplary Embodiment 26: The method of embodiment 25, wherein the cancer nucleic acid molecules are derived from a tumor portion of a heterogeneous tissue biopsy sample, and the non-cancer nucleic acid molecules are derived from a normal portion of the heterogeneous tissue biopsy sample.

Exemplary Embodiment 27: The method of embodiment 25, wherein the sample comprises a liquid biopsy sample, and wherein the cancer nucleic acid molecules are derived from a circulating tumor DNA (ctDNA) fraction of the liquid biopsy sample, and the non-cancer nucleic acid molecules are derived from a non-tumor fraction of the liquid biopsy sample.

Exemplary Embodiment 28: The method of any one of embodiments 20-23 and 25-27, wherein the one or more adapters comprise amplification primers, flow cell adapter sequences, substrate adapter sequences, sample index sequences, or unique molecular identifier (UMI) sequences.

Exemplary Embodiment 29: The method of any one of embodiments 24-27, wherein the selectively enriching comprises: (a) combining one or more bait molecules with the library, thereby hybridizing the one or more bait molecules to one or more nucleic acid molecules comprising nucleotide sequences corresponding to the RET fusion nucleic acid molecule and producing nucleic acid hybrids; and (b) isolating the nucleic acid hybrids to produce the enriched sample.

Exemplary Embodiment 30: The method of any one of embodiments 20-23 and 25-28, wherein the captured nucleic acid molecules are captured from the amplified nucleic acid molecules by hybridization to one or more bait molecules.

Exemplary Embodiment 31: The method of any one of embodiments 20-30, wherein the amplifying comprises performing a polymerase chain reaction (PCR) amplification technique, a non-PCR amplification technique, or an isothermal amplification technique.

Exemplary Embodiment 32: The method of any one of embodiments 20-31, wherein the sequencing comprises use of a massively parallel sequencing (MPS) technique, whole genome sequencing (WGS), whole exome sequencing, targeted sequencing, direct sequencing, or a Sanger sequencing technique.

Exemplary Embodiment 33: The method of embodiment 32, wherein the sequencing comprises a massively parallel sequencing technique, and the massively parallel sequencing technique comprises next generation sequencing (NGS).

Exemplary Embodiment 34: The method of any one of embodiments 20-23, 25-28, and 30-33, wherein the sequencer comprises a next generation sequencer.

Exemplary Embodiment 35: The method of any one of embodiments 20-34, further comprising generating a molecular profile for the individual, based, at least in part, on detecting the presence or absence of the RET fusion nucleic acid molecule.

Exemplary Embodiment 36: The method of embodiment 35, wherein the molecular profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof.

Exemplary Embodiment 37: The method of embodiment 35 or embodiment 36, wherein the molecular profile for the individual further comprises results from a nucleic acid sequencing-based test.

Exemplary Embodiment 38: The method of any one of embodiments 35-37, further comprising selecting a treatment, administering a treatment, or applying a treatment to the individual based on the generated molecular profile, wherein the treatment comprises a RET-targeted therapy.

Exemplary Embodiment 39: The method of any one of embodiments 20-38, further comprising generating a report indicating the presence or absence of the RET fusion nucleic acid molecule in the sample.

Exemplary Embodiment 40: The method of embodiment 21 or embodiment 22, further comprising generating, by the one or more processors, a report indicating the presence or absence of the RET fusion nucleic acid molecule in the sample.

Exemplary Embodiment 41: The method of embodiment 39 or embodiment 40, further comprising transmitting the report to the individual, a caregiver, a healthcare provider, a physician, an oncologist, an electronic medical record system, a hospital, a clinic, a third-party payer, an insurance company, or a government office.

Exemplary Embodiment 42: The method of embodiment 41, wherein the report is transmitted via a computer network or a peer-to-peer connection.

Exemplary Embodiment 43: A method of identifying a candidate treatment for a cancer in an individual in need thereof, comprising performing DNA sequencing on a sample obtained from the individual to determine a sequencing mutation profile, wherein the sequencing mutation profile identifies the presence or absence of a RET fusion nucleic acid molecule, wherein:
(i) the RET fusion nucleic acid molecule comprises a fusion between a RET gene, or a portion thereof, and a gene listed in Table 1, or a portion thereof, or
(ii) the RET fusion nucleic acid molecule comprises a fusion between a RET gene, or a portion thereof, and a gene listed in Table 2, or a portion thereof, and the cancer is the cancer corresponding to the gene listed in Table 2.

Exemplary Embodiment 44: The method of embodiment 43, wherein the candidate treatment comprises a RET-targeted therapy.

Exemplary Embodiment 45: The method of embodiment 43 or embodiment 44, wherein the presence of the RET fusion nucleic acid molecule in the sample identifies the individual as one who may benefit from a treatment comprising a RET-targeted therapy.

Exemplary Embodiment 46: The method of any one of embodiments 43-45, wherein the presence of the RET fusion nucleic acid molecule in the sample predicts the individual to have longer survival when treated with a treatment comprising a RET-targeted therapy, as compared to survival of an individual whose cancer does not comprise a RET fusion nucleic acid molecule.

Exemplary Embodiment 47: The method of any one of embodiments 43-46, wherein the sequencing comprises use of a massively parallel sequencing (MPS) technique, whole genome sequencing (WGS), whole exome sequencing, targeted sequencing, direct sequencing, or a Sanger sequencing technique.

Exemplary Embodiment 48: The method of embodiment 47, wherein the sequencing comprises a massively parallel sequencing technique, and the massively parallel sequencing technique comprises next generation sequencing (NGS).

Exemplary Embodiment 49: The method of any one of embodiments 43-48, wherein the sequencing mutation profile identifies the presence or absence of a fragment of the RET fusion nucleic acid molecule comprising a breakpoint or fusion junction.

Exemplary Embodiment 50: A method of treating or delaying progression of cancer, comprising:
(a) detecting a RET fusion nucleic acid molecule, or a RET fusion polypeptide encoded by the RET fusion nucleic acid molecule, in a sample from an individual having a cancer, wherein:
   (i) the RET fusion nucleic acid molecule comprises a fusion between a RET gene, or a portion thereof, and a gene listed in Table 1, or a portion thereof, or
   (ii) the RET fusion nucleic acid molecule comprises a fusion between a RET gene, or a portion thereof, and a gene listed in Table 2, or a portion thereof, and the cancer is the cancer corresponding to the gene listed in Table 2; and
(b) administering to the individual an effective amount of a treatment that comprises a RET-targeted therapy.

Exemplary Embodiment 51: The method of any one of embodiments 1-50, wherein the RET fusion nucleic acid molecule is a RET fusion nucleic acid molecule listed in Table 3, and wherein the order of the genes in the fusion, in 5′ to 3′ direction, is as listed in Table 3.

Exemplary Embodiment 52: The method of any one of embodiments 1-51, wherein the RET fusion nucleic acid molecule is a RET fusion nucleic acid molecule listed in Table 4, and wherein the RET fusion nucleic acid molecule comprises or results from a corresponding 5′ breakpoint and/or 3′ breakpoint within the exons or introns as listed in Table 4.

Exemplary Embodiment 53: The method of any one of embodiments 1-52, wherein the RET fusion nucleic acid molecule is a RET fusion nucleic acid molecule listed in Table 5, and wherein the RET fusion nucleic acid molecule comprises or results from a corresponding 5′ breakpoint within the chromosomal coordinates as listed in Table 5, and/or a corresponding 3′ breakpoint within the chromosomal coordinates as listed in Table 5.

Exemplary Embodiment 54: The method of any one of embodiments 1-53, wherein the RET fusion nucleic acid molecule is a RET fusion nucleic acid molecule listed in Table 6, and wherein the RET fusion nucleic acid molecule comprises or results from a fusion of a corresponding 5′ exon as listed in Table 6, or a portion thereof, fused to a corresponding 3′ exon as listed in Table 6, or a portion thereof.

Exemplary Embodiment 55: The method of any one of embodiments 1-54, wherein the RET fusion nucleic acid molecule is a RET fusion nucleic acid molecule listed in Table 7, and wherein the RET fusion nucleic acid molecule comprises, in 5′ to 3′ direction, the corresponding exons or portions thereof as listed in Table 7.

Exemplary Embodiment 56: The method of any one of embodiments 1-55, wherein the RET fusion nucleic acid molecule is a RET fusion nucleic acid molecule listed in Table 8, and wherein the RET fusion nucleic acid molecule comprises a corresponding nucleotide sequence as listed in Table 8, or a nucleotide sequence with at least about 70% homology thereto.

Exemplary Embodiment 57: The method of any one of embodiments 1-56, wherein the RET fusion nucleic acid molecule encodes a RET fusion polypeptide.

Exemplary Embodiment 58: The method of any one of embodiments 1-57, wherein the RET fusion nucleic acid molecule comprises a nucleotide sequence encoding a corresponding RET fusion polypeptide as listed in Table 9, wherein the RET fusion polypeptide comprises a corresponding amino acid sequence as listed in Table 9, or an amino acid sequence with at least about 70% homology thereto.

Exemplary Embodiment 59: The method of any one of embodiments 1-19 and 50-58, wherein the RET fusion polypeptide encoded by the RET fusion nucleic acid molecule comprises a RET kinase domain, or a fragment of a RET kinase domain having RET kinase activity.

Exemplary Embodiment 60: The method of any one of embodiments 1-19 and 50-59, wherein the RET fusion polypeptide encoded by the RET fusion nucleic acid molecule has RET kinase activity.

Exemplary Embodiment 61: The method of any one of embodiments 1-19 and 50-60, wherein the RET fusion polypeptide encoded by the RET fusion nucleic acid molecule has a constitutive RET kinase activity.

Exemplary Embodiment 62: The method of any one of embodiments 1-19 and 50-61, wherein the RET fusion polypeptide encoded by the RET fusion nucleic acid molecule is oncogenic.

Exemplary Embodiment 63: The method of any one of embodiments 1-19 and 50-62, wherein the RET fusion polypeptide encoded by the RET fusion nucleic acid molecule promotes cancer cell survival, angiogenesis, cancer cell proliferation, and any combination thereof.

Exemplary Embodiment 64: The method of any one of embodiments 1-19 and 50-63, wherein the RET fusion polypeptide encoded by the RET fusion nucleic acid molecule is capable of dimerizing with a RET polypeptide or with another RET fusion polypeptide.

Exemplary Embodiment 65: The method of any one of embodiments 1-19 and 50-64, wherein the RET fusion polypeptide encoded by the RET fusion nucleic acid molecule is a RET fusion polypeptide listed in Table 9, and wherein the RET fusion polypeptide comprises a corresponding amino acid sequence as listed in Table 9, or an amino acid sequence with at least about 70% homology thereto.

Exemplary Embodiment 66: The method of any one of embodiments 1-50, wherein the RET fusion nucleic acid molecule is a RET fusion nucleic acid molecule comprising a fusion between a RET gene, or a portion thereof, and a gene listed in Table 1, or a portion thereof, and wherein the cancer is a carcinoma, a sarcoma, a lymphoma, a leukemia, a myeloma, a germ cell cancer, or a blastoma.

Exemplary Embodiment 67: The method of any one of embodiments 1-50 and 66, wherein the RET fusion nucleic acid molecule is a RET fusion nucleic acid molecule comprising a fusion between a RET gene, or a portion thereof, and a gene listed in Table 1, or a portion thereof, and wherein the cancer is a solid tumor.

Exemplary Embodiment 68: The method of any one of embodiments 1-50 and 66, wherein the RET fusion nucleic acid molecule is a RET fusion nucleic acid molecule comprising a fusion between a RET gene, or a portion thereof, and a gene listed in Table 1, or a portion thereof, and wherein the cancer is a hematologic malignancy.

Exemplary Embodiment 69: The method of any one of embodiments 1-50 and 66, wherein the RET fusion nucleic acid molecule is a RET fusion nucleic acid molecule comprising a fusion between a RET gene, or a portion thereof, and a gene listed in Table 1, or a portion thereof, and wherein the cancer is a lymphoma.

Exemplary Embodiment 70: The method of any one of embodiments 1-50 and 66, wherein the RET fusion nucleic acid molecule is a RET fusion nucleic acid molecule comprising a fusion between a RET gene, or a portion thereof, and a gene listed in Table 1, or a portion thereof, and wherein the cancer is:
an ovarian cancer, a thyroid cancer, an adenocarcinoma, a breast cancer, a lung cancer, a colon cancer, a carcinoma, a uterine cancer, a prostate cancer, a pancreatic cancer, a leiomyosarcoma, a sarcoma, an esophageal cancer, a brain cancer, a bladder cancer, a skin cancer, a cervical cancer, or a melanoma; or
ovary epithelial carcinoma, thyroid papillary carcinoma, unknown primary adenocarcinoma, breast carcinoma, lung non-small cell lung carcinoma, colon adenocarcinoma, unknown primary carcinoma, breast invasive ductal carcinoma, uterus endometrial adenocarcinoma mixed histology, prostate acinar adenocarcinoma, lung squamous cell carcinoma, lung small cell undifferentiated carcinoma, pancreas ductal adenocarcinoma, bladder urothelial (transitional cell) carcinoma, soft tissue leiomyosarcoma, soft tissue sarcoma, esophagus adenocarcinoma, ovary serous carcinoma, colon neuroendocrine carcinoma, brain glioblastoma, breast carcinoma, unknown primary malignant neoplasm, lung adenocarcinoma, unknown primary cancer, unknown primary serous carcinoma, thyroid carcinoma, uterus carcinosarcoma, pancreatobiliary carcinoma, unknown primary urothelial carcinoma, neuroendocrine tumor, unknown primary neuroendocrine tumor, brain astrocytoma, cholangiocarcinoma, intra-hepatic cholangiocarcinoma, cervix squamous cell carcinoma, or unknown primary melanoma.

Exemplary Embodiment 71: The method of any one of embodiments 1-50 and 66, wherein the RET fusion nucleic acid molecule is a RET fusion nucleic acid molecule comprising a fusion between a RET gene, or a portion thereof, and a gene listed in Table 1, or a portion thereof, and wherein the cancer is:
a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pincaloma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypercosinophilia, chronic cosinophilic leukemia, neuroendocrine cancers, or a carcinoid tumor; or
acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

Exemplary Embodiment 72: The method of any one of embodiments 1-50 and 66, wherein the RET fusion nucleic acid molecule is a RET fusion nucleic acid molecule comprising a fusion between a RET gene, or a portion thereof, and a gene listed in Table 1, or a portion thereof, and wherein:
(a) the RET fusion nucleic acid molecule is a RET fusion nucleic acid molecule as listed in Table 3, and wherein the order of the genes in the fusion, in 5′ to 3′ direction, is as listed in Table 3:
(b) the RET fusion nucleic acid molecule is a RET fusion nucleic acid molecule as listed in Table 4, and wherein the RET fusion nucleic acid molecule comprises or results from a corresponding 5′ breakpoint and/or 3′ breakpoint within the exons or introns as listed in Table 4:
(c) the RET fusion nucleic acid molecule is a RET fusion nucleic acid molecule as listed in Table 5, and wherein the RET fusion nucleic acid molecule comprises or results from a corresponding 5′ breakpoint within the chromosomal coordinates as listed in Table 5, and/or a corresponding 3′ breakpoint within the chromosomal coordinates as listed in Table 5;
(d) the RET fusion nucleic acid molecule is a RET fusion nucleic acid molecule as listed in Table 6, and wherein the RET fusion nucleic acid molecule comprises or results from a fusion of a corresponding 5′ exon as listed in Table 6, or a portion thereof, fused to a corresponding 3′ exon as listed in Table 6, or a portion thereof;
(e) the RET fusion nucleic acid molecule is a RET fusion nucleic acid molecule as listed in Table 7, and wherein the RET fusion nucleic acid molecule comprises, in 5′ to 3′ direction, the corresponding exons or portions thereof as listed in Table 7;
(f) the RET fusion nucleic acid molecule is a RET fusion nucleic acid molecule as listed in Table 8, and wherein the RET fusion nucleic acid molecule comprises a corresponding nucleotide sequence as listed in Table 8, or a nucleotide sequence with at least about 70% homology thereto; and/or
(g) the RET fusion nucleic acid molecule comprises a nucleotide sequence encoding a corresponding RET fusion polypeptide as listed in Table 9, wherein the RET fusion polypeptide comprises a corresponding amino acid sequence as listed in Table 9, or an amino acid sequence with at least about 70% homology thereto; and
wherein the cancer is the corresponding cancer as listed in Table 10.

Exemplary Embodiment 73: The method of any one of embodiments 1-72, wherein the cancer is metastatic.

Exemplary Embodiment 74: The method of any one of embodiments 1-11, 19, 38-39, 41-42, and 44-73, wherein the RET-targeted therapy comprises one or more of a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for RET-positive or RET-rearranged cancer, a RET-targeted therapy being tested in a clinical trial, a treatment for RET-positive or RET-rearranged cancer being tested in a clinical trial, or any combination thereof.

Exemplary Embodiment 75: The method of any one of embodiments 1-11, 19, 38-39, 41-42, and 44-74, wherein the RET-targeted therapy is a kinase inhibitor.

Exemplary Embodiment 76: The method of embodiment 75, wherein the RET-targeted therapy is a tyrosine kinase inhibitor.

Exemplary Embodiment 77: The method of embodiment 75 or embodiment 76, wherein the RET-targeted therapy is kinase inhibitor that inhibits the kinase activity of a RET polypeptide.

Exemplary Embodiment 78: The method of any one of embodiments 75-77, wherein the RET-targeted therapy is a multi-kinase inhibitor or a RET-specific inhibitor.

Exemplary Embodiment 79: The method of any one of embodiments 75-78, wherein the RET-targeted therapy comprises one or more of pralsetinib, selpercatinib, lenvatinib, sorafenib, sunitinib, vandetanib, NVP-AST487, regorafenib, dovitinib, motesanib, cabozantinib, lapatinib, lestaurtinib, linifanib, semaxinib, ponatinib, fostamatinib, quizartinib, imatinib, vatalanib, ENMD-2076, JNJ-26483327, DCC-2157, Zeteletinib, TPX0046, TAS0953, RXDX-105, LOXO-260, BOS172738, Alectinib, APS03118, LOX-18228, or SYHA1815.

Exemplary Embodiment 80: The method of embodiment 74, wherein the nucleic acid inhibits the expression of the RET fusion nucleic acid molecule or the RET fusion polypeptide encoded by the RET fusion nucleic acid molecule.

Exemplary Embodiment 81: The method of embodiment 80, wherein the nucleic acid is a double-stranded RNA (dsRNA), a small interfering RNA (siRNA), or a small hairpin RNA (shRNA).

Exemplary Embodiment 82: The method of embodiment 74, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Embodiment 83: The method of any one of embodiments 1-82, wherein the individual has received a prior anti-cancer treatment, or is being treated with an anti-cancer treatment.

Exemplary Embodiment 84: The method of embodiment 83, wherein the RET fusion nucleic acid molecule, and/or the RET fusion polypeptide encoded by the RET fusion nucleic acid molecule, confers resistance of the cancer to the anti-cancer treatment.

Exemplary Embodiment 85: The method of embodiment 83 or embodiment 84, wherein the anti-cancer treatment is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer being tested in a clinical trial, an immunotherapy, a chemotherapy, a targeted therapy, a non-RET-targeted anti-cancer therapy, or any combination thereof.

Exemplary Embodiment 86: The method of embodiment 85, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Embodiment 87: The method of embodiment 85, wherein the nucleic acid comprises a double-stranded RNA (dsRNA), a small interfering RNA (siRNA), or a small hairpin RNA (shRNA).

Exemplary Embodiment 88: The method of any one of embodiments 1-82, wherein the cancer has not been previously treated.

Exemplary Embodiment 89: The method of any one of embodiments 1-11, 19. 38-39, 41-42, 44-82, and 88, wherein the RET-targeted therapy is a first-line or front-line treatment.

Exemplary Embodiment 90: The method of embodiment 88 or embodiment 89, wherein the RET-targeted therapy comprises one or more of pralsetinib, selpercatinib, lenvatinib, sorafenib, sunitinib, vandetanib, NVP-AST487, regorafenib, dovitinib, motesanib, cabozantinib, lapatinib, lestaurtinib, linifanib, semaxinib, ponatinib, fostamatinib, quizartinib, imatinib, vatalanib, ENMD-2076, JNJ-26483327, DCC-2157, Zeteletinib, TPX0046, TAS0953, RXDX-105, LOXO-260, BOS172738, Alectinib, APS03118, LOX-18228, or SYHA1815.

Exemplary Embodiment 91: The method of any one of embodiments 1-82 and 88-90, wherein the cancer is kinase inhibitor-naïve.

Exemplary Embodiment 92: The method of any one of embodiments 1-82 and 88-91, wherein the cancer has not been previously treated with a kinase inhibitor.

Exemplary Embodiment 93: The method of any one of embodiments 1-87, wherein the cancer has been previously treated with a kinase inhibitor.

Exemplary Embodiment 94: The method of any one of embodiments 1-87 and 93, wherein the cancer progressed on a prior treatment with a kinase inhibitor.

Exemplary Embodiment 95: The method of any one of embodiments 1-87 and 93-94.

wherein the cancer is refractory to a prior kinase inhibitor treatment.

Exemplary Embodiment 96: The method of any one of embodiments 1-87 and 93-95, wherein the cancer progressed on a prior treatment with a chemotherapy and a kinase inhibitor.

Exemplary Embodiment 97: The method of any one of embodiments 93-96, wherein the kinase inhibitor is a tyrosine kinase inhibitor.

Exemplary Embodiment 98: The method of any one of embodiments 93-97, wherein the kinase inhibitor inhibits the kinase activity of a RET polypeptide.

Exemplary Embodiment 99: The method of any one of embodiments 93-98, wherein the kinase inhibitor is a multi-kinase inhibitor or a RET-specific inhibitor.

Exemplary Embodiment 100: The method of any one of embodiments 93-99, wherein the RET-targeted therapy comprises one or more of pralsetinib, selpercatinib, lenvatinib, sorafenib, sunitinib, vandetanib, NVP-AST487, regorafenib, dovitinib, motesanib, cabozantinib, lapatinib, lestaurtinib, linifanib, semaxinib, ponatinib, fostamatinib, quizartinib, imatinib, vatalanib, ENMD-2076, JNJ-26483327, DCC-2157, Zeteletinib, TPX0046, TAS0953, RXDX-105, LOXO-260, BOS172738, Alectinib, APS03118, LOX-18228, or SYHA1815.

Exemplary Embodiment 101: The method of any one of embodiments 1-11, 19, 38-39, 41-42, and 44-100, wherein the treatment or the one or more treatment options further comprise an additional anti-cancer therapy.

Exemplary Embodiment 102: The method of embodiment 101, wherein the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

Exemplary Embodiment 103: The method of embodiment 102, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Embodiment 104: The method of embodiment 102, wherein the nucleic acid comprises a double-stranded RNA (dsRNA), a small interfering RNA (siRNA), or a small hairpin RNA (shRNA).

Exemplary Embodiment 105: The method of any one of embodiments 1-104, further comprising obtaining the sample from the individual.

Exemplary Embodiment 106: The method of any one of embodiments 1-105, wherein the sample is obtained from the cancer.

Exemplary Embodiment 107: The method of any one of embodiments 1-106, wherein the sample comprises a tissue biopsy sample, a liquid biopsy sample, or a normal control.

Exemplary Embodiment 108: The method of embodiment 107, wherein the sample is from a tumor biopsy, tumor specimen, or circulating tumor cell.

Exemplary Embodiment 109: The method of any one of embodiments 1-107, wherein the sample is a liquid biopsy sample and comprises blood, plasma, cerebrospinal fluid, sputum, stool, urine, or saliva.

Exemplary Embodiment 110: The method of any one of embodiments 1-109, wherein the sample comprises cells and/or nucleic acids from the cancer.

Exemplary Embodiment 111: The method of embodiment 110, wherein the sample comprises mRNA. DNA, circulating tumor DNA (ctDNA), cell-free DNA, or cell-free RNA from the cancer.

Exemplary Embodiment 112: The method of embodiment 109, wherein the sample is a liquid biopsy sample and comprises circulating tumor cells (CTCs).

Exemplary Embodiment 113: The method of embodiment 109, wherein the sample is a liquid biopsy sample and comprises cell-free DNA (cfDNA), circulating tumor DNA (ctDNA), or any combination thereof.

Exemplary Embodiment 114: The method of any one of embodiments 1-113, comprising acquiring knowledge of or detecting the RET fusion nucleic acid molecule or the RET fusion polypeptide encoded by the RET fusion nucleic acid molecule in a tissue biopsy sample, in a liquid biopsy sample, or in both a tissue biopsy sample and a liquid biopsy sample, from the individual.

Exemplary Embodiment 115: The method of any one of embodiments 4-11 and 51-114, wherein the acquiring knowledge of the RET fusion nucleic acid molecule or the RET fusion polypeptide encoded by the RET fusion nucleic acid molecule comprises detecting the RET fusion nucleic acid molecule, or the RET fusion polypeptide encoded by the RET fusion nucleic acid molecule, in the sample.

Exemplary Embodiment 116: The method of any one of embodiments 1-3, 12-42, and 50-115, wherein detecting the RET fusion nucleic acid molecule in the sample comprises detecting: (i) a fragment of the RET fusion nucleic acid molecule comprising a breakpoint or fusion junction between the RET gene, or the portion thereof, and the gene listed in Table 1, or the portion thereof, or (ii) a fragment of the RET fusion nucleic acid molecule comprising a breakpoint or fusion junction between the RET gene, or the portion thereof, and the gene listed in Table 2, or the portion thereof.

Exemplary Embodiment 117: The method of any one of embodiments 1-3, 12-19, and 50-116, wherein the RET fusion nucleic acid molecule is detected in the sample by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing.

Exemplary Embodiment 118: The method of embodiment 117, wherein the sequencing comprises a massively parallel sequencing (MPS) technique, whole genome sequencing (WGS), whole exome sequencing, targeted sequencing, direct sequencing, or a Sanger sequencing technique; and optionally wherein the massively parallel sequencing (MPS) technique comprises next-generation sequencing (NGS).

Exemplary Embodiment 119: The method of any one of embodiments 1-3, 12-19, and 50-115, wherein detecting the RET fusion polypeptide encoded by the RET fusion nucleic acid molecule comprises detecting a fragment of the RET fusion polypeptide that is encoded by: (i) a fragment of the RET fusion nucleic acid molecule that comprises a breakpoint or fusion junction between the RET gene, or the portion thereof, and the gene listed in Table 1, or the portion thereof, or (ii) a fragment of the RET fusion nucleic acid molecule that comprises a breakpoint or fusion junction between the RET gene, or the portion thereof, and the gene listed in Table 2, or the portion thereof.

Exemplary Embodiment 120: The method of any one of embodiments 1-3, 12-19, 50-115, and 119, wherein the RET fusion polypeptide encoded by the RET fusion nucleic acid molecule is detected in the sample by one or more of: immunoblotting, enzyme linked immunosorbent assay (ELISA), immunohistochemistry, or mass spectrometry.

Exemplary Embodiment 121: The method of any one of embodiments 1-3, 12-19, and 50-118, further comprising selectively enriching for one or more nucleic acid molecules in the sample comprising nucleotide sequences corresponding to the RET fusion nucleic acid molecule; wherein the selectively enriching produces an enriched sample.

Exemplary Embodiment 122: The method of embodiment 121, wherein the selectively enriching comprises: (a) combining one or more bait molecules with the sample, thereby hybridizing the one or more bait molecules to one or more nucleic acids in the sample comprising nucleotide sequences corresponding to the RET fusion nucleic acid molecule and producing nucleic acid hybrids; and (b) isolating the nucleic acid hybrids to produce the enriched sample.

Exemplary Embodiment 123: The method of any one of embodiments 23, 29, 30, and 122, wherein the one or more bait molecules comprise a capture nucleic acid molecule configured to hybridize to a nucleotide sequence corresponding to the RET fusion nucleic acid molecule.

Exemplary Embodiment 124: The method of embodiment 123, wherein the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides.

Exemplary Embodiment 125: The method of any one of embodiments 23, 29, 30, and 122-124, wherein the one or more bait molecules are conjugated to an affinity reagent or to a detection reagent.

Exemplary Embodiment 126: The method of embodiment 125, wherein the affinity reagent is an antibody, an antibody fragment, or biotin, or wherein the detection reagent is a fluorescent marker.

Exemplary Embodiment 127: The method of any one of embodiments 123-126, wherein the capture nucleic acid molecule comprises a DNA, RNA, or mixed DNA/RNA molecule.

Exemplary Embodiment 128: The method of embodiment 24 or embodiment 121, wherein the selectively enriching comprises amplifying the one or more nucleic acid molecules comprising nucleotide sequences corresponding to the RET fusion nucleic acid molecule using a polymerase chain reaction (PCR) to produce an enriched sample.

Exemplary Embodiment 129: The method of any one of embodiments 121-128, further comprising sequencing the enriched sample.

Exemplary Embodiment 130: The method of any one of embodiments 1-129, further comprising acquiring knowledge of or detecting in a sample from the individual a base substitution, a short insertion/deletion (indel), a copy number alteration, or a genomic rearrangement in one or more genes.

Exemplary Embodiment 131: The method of any one of embodiments 1-130, wherein the individual is a human.

Exemplary Embodiment 132: A kit comprising one or more probes, baits, and/or oligonucleotides for detecting:
(i) a RET fusion nucleic acid molecule comprising a fusion between a RET gene, or a portion thereof, and a gene listed in Table 1, or a portion thereof, or
(ii) a RET fusion nucleic acid molecule comprising a fusion between a RET gene, or a portion thereof, and a gene listed in Table 2, or a portion thereof, in a corresponding cancer as listed in Table 2.

Exemplary Embodiment 133: A nucleic acid encoding a RET fusion nucleic acid molecule, or a fragment thereof, comprising a fusion between a RET gene, or a portion thereof, and a gene listed in Table 1, or a portion thereof.

Exemplary Embodiment 134: A vector comprising the nucleic acid of embodiment 133.

Exemplary Embodiment 135: A host cell comprising the vector of embodiment 134.

Exemplary Embodiment 136: An antibody or antibody fragment that specifically binds to a RET fusion polypeptide, or to a portion thereof, wherein the RET fusion polypeptide is encoded by a RET fusion nucleic acid molecule comprising a fusion between a RET gene, or a portion thereof, and a gene listed in Table 1, or a portion thereof.

Exemplary Embodiment 137: A kit comprising the antibody or antibody fragment of embodiment 136.

Exemplary Embodiment 138: In vitro use of one or more probes, baits, and/or oligonucleotides for detecting:
(i) a RET fusion nucleic acid molecule comprising a fusion between a RET gene, or a portion thereof, and a gene listed in Table 1, or a portion thereof, or
(ii) a RET fusion nucleic acid molecule comprising a fusion between a RET gene, or a portion thereof, and a gene listed in Table 2, or a portion thereof, in a corresponding cancer as listed in Table 2.

Exemplary Embodiment 139: A system, comprising:
a memory configured to store one or more program instructions, and one or more processors configured to execute the one or more program instructions, the one or more program instructions when executed by the one or more processors are configured to:
(a) obtain a plurality of sequence reads of one or more nucleic acid molecules, wherein the one or more nucleic acid molecules are derived from a sample obtained from an individual having a cancer;
(b) analyze the plurality of sequence reads for the presence of a RET fusion nucleic acid molecule, wherein:
   (i) the RET fusion nucleic acid molecule comprises a fusion between a RET gene, or a portion thereof, and a gene listed in Table 1, or a portion thereof, or
   (ii) the RET fusion nucleic acid molecule comprises a fusion between a RET gene, or a portion thereof, and a gene listed in Table 2, or a portion thereof, and wherein the cancer is the corresponding cancer as listed in Table 2; and
(c) detect, based on the analyzing, the RET fusion nucleic acid molecule in the sample.

Exemplary Embodiment 140: A non-transitory computer readable storage medium comprising one or more programs executable by one or more computer processors for performing a method, the method comprising:
(a) obtaining, using the one or more processors, a plurality of sequence reads of one or more nucleic acid molecules, wherein the one or more nucleic acid molecules are derived from a sample obtained from an individual having a cancer;
(b) analyzing, using the one or more processors, the plurality of sequence reads for the presence of a RET fusion nucleic acid molecule, wherein:
   (i) the RET fusion nucleic acid molecule comprises a fusion between a RET gene, or a portion thereof, and a gene listed in Table 1, or a portion thereof, or
   (ii) the RET fusion nucleic acid molecule comprises a fusion between a RET gene, or a portion thereof, and a gene listed in Table 2, or a portion thereof, and wherein the cancer is the corresponding cancer as listed in Table 2; and
(c) detecting, using the one or more processors and based on the analyzing, the RET fusion nucleic acid molecule in the sample.

Exemplary Embodiment 141: The system of embodiment 139, or the non-transitory computer readable storage medium of embodiment 140, wherein the plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next generation sequencing (NGS).

Exemplary Embodiment 142: The system of embodiment 139 or embodiment 141, wherein the one or more program instructions when executed by the one or more processors are further configured to generate, based at least in part on the detecting, a molecular profile for the sample.

Exemplary Embodiment 143: The non-transitory computer readable storage medium of embodiment 140 or embodiment 141, wherein the method further comprises generating, based at least in part on the detecting, a molecular profile for the sample.

Exemplary Embodiment 144: The system of embodiment 142, or the non-transitory computer readable storage medium of embodiment 143, wherein the individual is administered a treatment based at least in part on the molecular profile; optionally wherein the treatment comprises a RET-targeted therapy.

Exemplary Embodiment 145: The system of any one of embodiments 142 and 144, or the non-transitory computer readable storage medium of 143 or embodiment 144, wherein the molecular profile further comprises results from a comprehensive genomic profiling (CGP) test, a gene expression profiling test, a cancer hotspot panel test, a DNA methylation test, a DNA fragmentation test, an RNA fragmentation test, or any combination thereof.

Exemplary Embodiment 146: The system of any one of embodiments 142 and 144-145, or the non-transitory computer readable storage medium of any one of embodiments 143-145, wherein the molecular profile further comprises results from a nucleic acid sequencing-based test.

Exemplary Embodiment 147: A RET-targeted therapy for use in a method of treating or delaying progression of cancer, wherein the method comprises administering the RET-targeted therapy to an individual having a cancer, wherein a RET fusion nucleic acid molecule, or a RET fusion polypeptide encoded by the RET fusion nucleic acid molecule, is detected in a sample from the individual, and wherein:
(a) the RET fusion nucleic acid molecule comprises a fusion between a RET gene, or a portion thereof, and a gene listed in Table 1, or a portion thereof, or
(b) the RET fusion nucleic acid molecule comprises a fusion between a RET gene, or a portion thereof, and a gene listed in Table 2, or a portion thereof, and wherein the cancer is the corresponding cancer as listed in Table 2.

Exemplary Embodiment 148: A RET-targeted therapy for use in the manufacture of a medicament for treating or delaying progression of cancer, wherein the medicament is to be administered to an individual having a cancer, wherein a RET fusion nucleic acid molecule, or a RET fusion polypeptide encoded by the RET fusion nucleic acid molecule, is detected in a sample from the individual, and wherein:
(a) the RET fusion nucleic acid molecule comprises a fusion between a RET gene, or a portion thereof, and a gene listed in Table 1, or a portion thereof, or
(b) the RET fusion nucleic acid molecule comprises a fusion between a RET gene, or a portion thereof, and a gene listed in Table 2, or a portion thereof, and wherein the cancer is the corresponding cancer as listed in Table 2.

The method steps of the invention(s) described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

The terminology used in the description of the various described embodiments herein is for the purpose of describing particular embodiments only and is not intended to be limiting. As used in the description of the various described embodiments and the appended claims, the singular forms “a.” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including.” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes. To the extent that any reference incorporated by reference conflicts with the instant disclosure, the instant disclosure shall control.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Identification of Novel RET Fusions in Cancer

This Example describes the identification and characterization of novel RET (Rearranged during transfection) gene fusions in cancer.

RET gene fusions were identified in a database of comprehensive genomic profiling (CGP) results (see, e.g., Frampton, G. M., et al. (2013) Nat. Biotechnol. 31 (11): 1023-31) of samples from patients with various cancer types. The CGP database was queried for RET gene fusions using Structured Query Language (SQL). The query was limited to gene fusions, and excluded out-of-strand rearrangements.

As shown in Table 11, RET gene fusions involving numerous gene fusion partners were identified.

TABLE 11
RET gene fusions identified by CGP.

| RET Fusion | Number of Cases |
|---|---|
| Partner Gene | Identified |
| CPEB3 | 2 |
| ADCY1 | 1 |
| ZNF248 | 1 |
| GOLGB1 | 1 |
| GP2 | 1 |
| LINC00379 | 1 |
| OPTN | 1 |
| PPP2R5A | 1 |
| HSD17B7P2 | 1 |
| MKX | 1 |
| NTRK2 | 1 |
| SH2D3A | 1 |
| ZNF721 | 1 |
| CCNY | 1 |
| GRM7 | 1 |
| OXR1 | 1 |
| MYH14 | 1 |
| AGBL4 | 1 |
| LRMDA | 1 |
| MGEA5 | 1 |
| SAMD4A | 1 |
| ACPP | 1 |
| BMS1 | 1 |
| ARHGAP19 | 1 |
| RBMS3 | 1 |
| SGIP1 | 1 |
| DCC | 1 |
| ELMO1 | 1 |
| CEP135 | 1 |
| EEA1 | 1 |
| CCBE1 | 1 |
| ADAMTS14 | 1 |
| RAD1 | 1 |
| RAI14 | 1 |
| NAALADL2 | 1 |
| ABI3BP | 1 |
| VSTM4 | 1 |
| ZSWIM6 | 1 |
| WDFY4 | 1 |

Additional RET gene fusions identified by CGP, and the corresponding cancer types in which the fusions were identified, are provided in Table 12.

TABLE 12
Additional RET gene fusions identified by CGP.

| | | Number of |
|---|---|---|
| RET Fusion | | Cases |
| Partner Gene | Tumor Type | Identified |
| SORBS1 | Soft tissue leiomyosarcoma | 2 |
| CSGALNACT2 | Uterus carcinosarcoma | 1 |
| CSGALNACT2 | Bladder urothelial (transitional | 1 |
| | cell) carcinoma | |
| PCM1 | Brain glioblastoma (GBM) | 1 |
| PCM1 | Pancreas ductal adenocarcinoma | 1 |
| KIAA1217 | Pancreas ductal adenocarcinoma | 1 |
| MPRIP | Soft tissue sarcoma (NOS) | 1 |
| TFG | Pancreas ductal adenocarcinoma | 1 |
| SPECC1L | Pancreas ductal adenocarcinoma | 1 |
| REEP3 | Esophagus adenocarcinoma | 1 |
| RRBP1 | Ovary serous carcinoma | 1 |
| ETV6 | Pancreas ductal adenocarcinoma | 1 |
| TAF3 | Colon neuroendocrine carcinoma | 1 |
| RASGEF1A | Unknown primary melanoma | 1 |
| TNIP2 | Colon adenocarcinoma (CRC) | 1 |
| SATB1 | Pancreas ductal adenocarcinoma | 1 |
| ALOX5 | Brain glioblastoma (GBM) | 1 |

A detailed characterization of each of the identified RET gene fusions was undertaken. As shown in Table 13, the identified RET gene fusions were present in diverse cancer types, with various fusion partner genes and breakpoints.

TABLE 13
Detailed characteristics of RET gene fusions identified by CGP.

| | 5′ breakpoint/ | | Fusion transcript |
|---|---|---|---|
| Fusion | 3′ breakpoint | | sequence |
| Exon and breakpoint information | 5′ Exons | 5′ Refseq ID/ | Fusion protein |
| Tumor type | 3′ Exons | 3′ Refseq ID | sequence |
| GOLGB1 - RET | chr3: 121428577- | NM_004487/ | SEQ ID NO: 2 |
| 5′-GOLGB1(ex1-10 NM_004487)- | 121428818/chr10: | NM_020630 | SEQ ID NO: 68 |
| RET(ex12-19 NM_020630) | 43611900-43612184 | | |
| Breakpoints GOLGB1 intron 10, RET | GOLGB1 ex1-10 | | |
| intron 11 | RET ex12-19 | | |
| Thyroid papillary carcinoma | | | |
| In frame fusion, includes RET kinase | | | |
| domain. | | | |
| GP2 - RET | chr16: 20331978- | NM_001502/ | SEQ ID NO: 3 |
| 5′-GP2(ex1-4 NM_001502)-RET(ex11- | 20332315/ | NM_020630 | SEQ ID NO: 69 |
| 19 NM_020630) Breakpoints GP2 | chr10: 43609279- | | |
| intron 4, RET intron 10 | 43609727 | | |
| Unknown primary adenocarcinoma | GP2 ex1-4 | | |
| In frame fusion, includes RET kinase | RET ex11-19 | | |
| domain. | | | |
| PPP2R5A - RET | chr1: 212474140- | NM_006243/ | SEQ ID NO: 4 |
| 5′-PPP2R5A(ex1-1 NM_006243)- | 212474319/ | NM_020975 | SEQ ID NO: 70 |
| RET(ex20-20 NM_020975) | chr10: 43623512- | | |
| Breakpoints PPP2R5A intron 1, RET | 43623678 | | |
| intron 19 | PPP2R5A ex1-1 | | |
| Breast carcinoma (NOS) | RET ex20-20 | | |
| In frame fusion. | | | |
| GRM7 - RET | chr3: 7728019- | NM_000844/ | SEQ ID NO: 6 |
| 5′-GRM7(ex1-9 NM_000844)- | 7728192/ | NM_020630 | SEQ ID NO: 72 |
| RET(ex3-19 NM_020630) Breakpoints | chr10: 43595834- | | |
| RET intron 2, GRM7 intron 9 | 43596313 | | |
| Colon adenocarcinoma (CRC) | GRM7 ex1-9 | | |
| In frame fusion, includes RET kinase | RET ex3-19 | | |
| domain. | | | |
| MYH14 - RET | chr19: 50801162- | NM_024729/ | SEQ ID NO: 8 |
| 5′-MYH14(ex1-37 NM_024729)- | 50801465/ | NM_020630 | SEQ ID NO: 74 |
| RET(ex12-19 NM_020630) | chr10: 43610285- | | |
| Breakpoints MYH14 intron 37, RET | 43610748 | | |
| intron 11 | MYH14 ex1-37 | | |
| Breast invasive ductal carcinoma (IDC) | RET ex12-19 | | |
| In frame fusion, includes RET kinase | | | |
| domain. | | | |
| MGEA5 - RET | chr10: 103551711- | NM_012215/ | SEQ ID NO: 9 |
| 5′-MGEA5(ex1-12 NM_012215)- | 103551879/ | NM_020630 | SEQ ID NO: 75 |
| RET(ex12-19 NM_020630) | chr10: 43610920- | | |
| Breakpoints MGEA5 intron 12, RET | 43611146 | | |
| intron 11 | MGEA5 ex1-12 | | |
| Ovary epithelial carcinoma (NOS) | RET ex12-19 | | |
| In frame fusion, includes RET kinase | | | |
| domain. | | | |
| SAMD4A - RET | chr14: 55123072- | NM_015589/ | SEQ ID NO: 10 |
| 5′-SAMD4A(ex1-2 NM_015589)- | 55123234/ | NM_020630 | SEQ ID NO: 76 |
| RET(ex11-19 NM_020630) | chr10: 43609336- | | |
| Breakpoints SAMD4A intron 2, RET | 43609516 | | |
| intron 10 | SAMD4A ex1-2 | | |
| Uterus endometrial adenocarcinoma | RET ex11-19 | | |
| mixed histology | | | |
| In frame fusion, includes RET kinase | | | |
| domain. | | | |
| ACPP - RET | chr3: 132078976- | NM_001134194/ | SEQ ID NO: 11 |
| 5′-ACPP(ex1-10 NM_001134194)- | 132079163/ | NM_020630 | SEQ ID NO: 77 |
| RET(ex11-19 NM_020630) | chr10: 43609901- | | |
| Breakpoints ACPP intron 10, RET | 43610079 | | |
| intron 10 | ACPP ex1-10 | | |
| Prostate acinar adenocarcinoma | RET ex11-19 | | |
| In frame fusion, includes RET kinase | | | |
| domain. | | | |
| BMS1 - RET | chr10: 43307628- | NM_014753/ | SEQ ID NO: 12 |
| 5′-BMS1(ex1-13 NM_014753)- | 43307807/ | NM_020630 | SEQ ID NO: 78 |
| RET(ex10-19 NM_020630) | chr10: 43608772- | | |
| Breakpoints BMS1 intron 13, RET | 43609039 | | |
| intron 9 | BMS1 ex1-13 | | |
| Lung squamous cell carcinoma (SCC) | RET ex 10-19 | | |
| In frame fusion, includes RET kinase | | | |
| domain. | | | |
| CEP135 - RET | chr4: 56844220- | NM_025009/ | SEQ ID NO: 13 |
| 5′-CEP135(ex1-11 NM_025009)- | 56844527/ | NM_020630 | SEQ ID NO: 79 |
| RET(ex12-19 NM_020630) | chr10: 43611070- | | |
| Breakpoints CEP135 intron 11, RET | 43611610 | | |
| intron 11 | CEP135 ex1-11 | | |
| Lung small cell undifferentiated | RET ex12-19 | | |
| carcinoma | | | |
| In frame fusion, includes RET kinase | | | |
| domain. | | | |
| EEA1- RET | chr12: 93218451- | NM_003566/ | SEQ ID NO: 14 |
| 5′-EEA1(ex1-13 NM_003566)- | 93218763/ | NM_020630 | SEQ ID NO: 80 |
| RET(ex12-19 NM_020630) | chr10: 43611264- | | |
| Breakpoints EEA1 intron 13, RET | 43611592 | | |
| intron 11 | EEA1 ex1-13 | | |
| Pancreas ductal adenocarcinoma | RET ex12-19 | | |
| In frame fusion, includes RET kinase | | | |
| domain. | | | |
| GSGALNACT2 - RET | chr10: 43671043- | NM_018590/ | SEQ ID NO: 15 |
| 5′-CSGALNACT2(ex1-6 NM_018590)- | 43671216/ | NM_020630 | SEQ ID NO: 81 |
| RET(ex12-19 NM_020630) | chr10: 43611674- | | |
| Breakpoints CSGALNACT2 intron 6, | 43611871 | | |
| RET intron 11 | CSGALNACT2 ex1-6 | | |
| Bladder urothelial (transitional cell) | RET ex12-19 | | |
| carcinoma | | | |
| In frame fusion, includes RET kinase | | | |
| domain. | | | |
| KIAA1217 - RET | chr10: 24810035- | NM_019590/ | SEQ ID NO: 16 |
| 5′-KIAA1217(ex1-11 NM_019590)- | 24810374/ | NM_020630 | SEQ ID NO: 82 |
| RET(ex8-19 NM_020630) Breakpoints | chr10: 43607337- | | |
| KIAA1217 intron 11, RET intron 7 | 43607677 | | |
| Pancreas ductal adenocarcinoma | KIAA1217 ex1-11 | | |
| In frame fusion, includes RET kinase | RET ex8-19 | | |
| domain. | | | |
| SORBS1 - RET | chr10: 97098893- | NM_006434/ | SEQ ID NO: 17 |
| 5′-SORBS1(ex1-20 NM_006434)- | 97098933/ | NM_020630 | SEQ ID NO: 83 |
| RET(ex7-19 NM_020630) Breakpoints | chr10: 43606666- | | |
| SORBS1 intron 20, RET intron 6 | 43606706 | | |
| Soft tissue leiomyosarcoma | SORBS1 ex1-20 | | |
| In frame fusion, includes RET kinase | RET ex7-19 | | |
| domain. | | | |
| SORBS1 - RET | chr10: 97077294- | NM_006434/ | SEQ ID NO: 18 |
| 5′-SORBS1(ex1-22 NM_006434)- | 97077537/ | NM_020630 | SEQ ID NO: 84 |
| RET(ex11-19 NM_020630) | chr10: 43609629- | | |
| Breakpoints SORBS1 intron 22, RET | 43609972 | | |
| intron 10 | SORBS1 ex1-22 | | |
| Soft tissue leiomyosarcoma | RET ex11-19 | | |
| In frame fusion, includes RET kinase | | | |
| domain. | | | |
| MPRIP - RET | chr17: 17083314- | NM_015134/ | SEQ ID NO: 19 |
| 5′-MPRIP(ex1-22 NM_015134)- | 17083354/ | NM_020630 | SEQ ID NO: 85 |
| RET(ex12-19 NM_020630) | chr10: 43612074- | | |
| Breakpoints MPRIP intron 22, RET | 43612114 | | |
| intron 11 | MPRIP ex1-22 | | |
| Soft tissue sarcoma (NOS) | RET ex12-19 | | |
| In frame fusion, includes RET kinase | | | |
| domain. | | | |
| TFG - RET | chr3: 100453395- | NM_006070/ | SEQ ID NO: 20 |
| 5′-TFG(ex1-5 NM_006070)-RET(ex11- | 100453864/ | NM_020630 | SEQ ID NO: 86 |
| 19 NM_020630) Breakpoints TFG | chr10: 43609629- | | |
| intron 5, RET intron 10 | 43610065 | | |
| Pancreas ductal adenocarcinoma | TFG ex1-5 | | |
| In frame fusion, includes RET kinase | RET ex11-19 | | |
| domain. | | | |
| SPECC1L - RET | chr22: 24739286- | NM_015330/ | SEQ ID NO: 21 |
| 5′-SPECC1L(ex1-10 NM_015330)- | 24739616/ | NM_020630 | SEQ ID NO: 87 |
| RET(ex12-19 NM_020630) | chr10: 43611732- | | |
| Breakpoints SPECC1L intron 10, RET | 43612114 | | |
| intron 11 | SPECC1L ex1-10 | | |
| Pancreas ductal adenocarcinoma | RET ex12-19 | | |
| In frame fusion, includes RET kinase | | | |
| domain. | | | |
| REEP3 - RET | chr10: 65367361- | NM_001001330/ | SEQ ID NO: 22 |
| 5′-REEP3(ex1-5 NM_001001330)- | 65368009/ | NM_020630 | SEQ ID NO: 88 |
| RET(ex12-19 NM_020630) | chr10: 43611264- | | |
| Breakpoints REEP3 intron 5, RET | 43611758 | | |
| intron 11 | REEP3 ex1-5 | | |
| Esophagus adenocarcinoma | RET ex12-19 | | |
| In frame fusion, includes RET kinase | | | |
| domain. | | | |
| RRBP1 - RET | chr20: 17596315- | NM_004587/ | SEQ ID NO: 23 |
| 5′-RRBP1(ex1-22 NM_004587)- | 17596767/ | NM_020630 | SEQ ID NO: 89 |
| RET(ex12-19 NM_020630) | chr10: 43610785- | | |
| Breakpoints RRBP1 intron 22, RET | 43611339 | | |
| intron 11 | RRBP1 ex1-22 | | |
| Ovary serous carcinoma | RET ex12-19 | | |
| In frame fusion, includes RET kinase | | | |
| domain. | | | |
| ETV6 - RET | chr12: 12038351- | NM_001987/ | SEQ ID NO: 24 |
| 5′-ETV6(ex1-6 NM_001987)- | 12038584/ | NM_020630 | SEQ ID NO: 90 |
| RET(ex12-19 NM_020630) | chr10: 43611377- | | |
| Breakpoints ETV6 intron 6, RET intron | 43611586 | | |
| 11 | ETV6 ex1-6 | | |
| Pancreas ductal adenocarcinoma | RET ex 12-19 | | |
| In frame fusion, includes RET kinase | | | |
| domain. | | | |
| TAF3 - RET | chr10: 8010776- | NM_031923/ | SEQ ID NO: 25 |
| 5′-TAF3(ex1-3 NM_031923)- | 8011037/ | NM_020630 | SEQ ID NO: 91 |
| RET(ex12-19 NM_020630) | chr10: 43610288- | | |
| Breakpoints TAF3 intron 3, RET intron | 43610507 | | |
| 11 | TAF3 ex1-3 | | |
| Colon neuroendocrine carcinoma | RET ex12-19 | | |
| In frame fusion, includes RET kinase | | | |
| domain. | | | |
| PCM1 - RET | chr8: 17856613- | NM_006197/ | SEQ ID NO: 26 |
| 5′-PCM1(ex1-29 NM_006197)- | 17856789/ | NM_020630 | SEQ ID NO: 92 |
| RET(ex12-19 NM_020630) | chr10: 43611737- | | |
| Breakpoints PCM1 intron 29, RET | 43612079 | | |
| intron 11 | PCM1 ex1-29 | | |
| Brain glioblastoma (GBM) | RET ex12-19 | | |
| In frame fusion, includes RET kinase | | | |
| domain. | | | |
| PCM1 - RET | chr8: 17858876- | NM_006197/ | SEQ ID NO: 27 |
| 5′-PCM1(ex1-29 NM_006197)- | 17859548/ | NM_020630 | SEQ ID NO: 93 |
| RET(ex12-19 NM_020630) | chr10: 43610288- | | |
| Breakpoints PCM1 intron 29, RET | 43611131 | | |
| intron 11 | PCM1 ex1-29 | | |
| Pancreas ductal adenocarcinoma | RET ex12-19 | | |
| In frame fusion, includes RET kinase | | | |
| domain. | | | |
| TNIP2 - RET | chr4: 2745391- | NM_024309/ | SEQ ID NO: 28 |
| 5′-TNIP2(ex1-5 NM_024309)- | 2745953/ | NM_020630 | SEQ ID NO: 94 |
| RET(ex12-19 NM_020630) | chr10: 43611294- | | |
| Breakpoints TNIP2 intron 5, RET | 43612177 | | |
| intron 11 | TNIP2 ex1-5 | | |
| Colon adenocarcinoma (CRC) | RET ex12-19 | | |
| In frame fusion, includes RET kinase | | | |
| domain. | | | |
| SATB1 - RET | chr3: 18433247- | NM_002971/ | SEQ ID NO: 29 |
| 5′-SATB 1(ex1-7 NM_002971)- | 18433798/ | NM_020630 | SEQ ID NO: 95 |
| RET(ex12-19 NM_020630) | chr10: 43610364- | | |
| Breakpoints SATB1 intron 7, RET | 43611028 | | |
| intron 11 | SATB1 ex1-7 | | |
| Pancreas ductal adenocarcinoma | RET ex12-19 | | |
| In frame fusion, includes RET kinase | | | |
| domain. | | | |
| RET - ADCY1 | chr10: 43610900- | NM_020630/ | SEQ ID NO: 30 |
| 5′-RET(ex1-11 NM_020630)- | 43611038/ | NM_021116 | SEQ ID NO: 96 |
| ADCY1(ex12-20 NM_021116) | chr7: 45724501- | | |
| Breakpoints RET intron 11, ADCY1 | 45724758 | | |
| intron 11 | RET ex1-11 | | |
| Breast carcinoma (NOS) | ADCY1 ex12-20 | | |
| In frame fusion. | | | |
| RET - ZNF248 | chr10: 43610329- | NM_020630/ | SEQ ID NO: 31 |
| 5′-RET(ex1-11 NM_020630)- | 43610698/ | NM_021045 | SEQ ID NO: 97 |
| ZNF248(ex4-6 NM_021045) | chr10: 38143593- | | |
| Breakpoints RET intron 11, ZNF248 | 38143957 | | |
| intron 3 | RET ex1-11 | | |
| Unknown primary malignant neoplasm | ZNF248 ex4-6 | | |
| (NOS) | | | |
| In frame fusion. | | | |
| RET - AGBL4 | chr10: 43612164- | NM_020630/ | SEQ ID NO: 32 |
| 5′-RET(ex1-12 NM_020630)- | 43612322/ | NM_032785 | SEQ ID NO: 98 |
| AGBL4(ex3-14 NM_032785) | chr1: 50296066- | | |
| Breakpoints RET intron 12, AGBL4 | 50296164 | | |
| intron 2 | RET ex1-12 | | |
| Ovary serous carcinoma | AGBL4 ex3-14 | | |
| In frame fusion. | | | |
| RET - LRMDA | chr10: 43609359- | NM_020630/ | SEQ ID NO: 33 |
| 5′-RET(ex1-10 NM_020630)- | 43609695/ | NM_032024 | SEQ ID NO: 99 |
| LRMDA(ex6-6 NM_032024) | chr10: 78117734- | | |
| Breakpoints RET intron 10, C10orf11 | 78117976 | | |
| intron 5 | RET ex1-10 | | |
| Ovary serous carcinoma | LRMDA ex6-6 | | |
| In frame fusion. | | | |
| RET - ARHGAP19 | chr10: 43610326- | NM_020630/ | SEQ ID NO: 34 |
| 5′-RET(ex1-11 NM_020630)- | 43610687/ | NM_032900 | SEQ ID NO: 100 |
| ARHGAP19(ex7-12 NM_032900) | chr10: 99014615- | | |
| Breakpoints RET intron 11, | 99014923 | | |
| ARHGAP19 intron 6 | RET ex1-11 | | |
| Lung adenocarcinoma | ARHGAP19 ex7-12 | | |
| In frame fusion. | | | |
| RET - CPEB3 | chr10: 43610292- | NM_020630/ | SEQ ID NO: 35 |
| 5′-RET(ex1-11 NM_020630)- | 43610477/ | NM_014912 | SEQ ID NO: 101 |
| CPEB3(ex8-10 NM_014912) | chr10: 93857438- | | |
| Breakpoints RET intron 11, CPEB3 | 93857889 | | |
| intron 7 | RET ex1-11 | | |
| Lung adenocarcinoma | CPEB3 ex8-10 | | |
| In frame fusion. | | | |
| RET - DCC | chr10: 43608372- | NM_020630/ | SEQ ID NO: 36 |
| 5′-RET(ex1-9 NM_020630)-DCC(ex8- | 43608512/ | NM_005215 | SEQ ID NO: 102 |
| 29 NM_005215) Breakpoints RET | chr18: 50604698- | | |
| intron 9, DCC intron 7 | 50604780 | | |
| Lung adenocarcinoma | RET ex1-9 | | |
| In frame fusion. | DCC ex8-29 | | |
| RET - ELMO1 | chr10: 43611573- | NM_020630/ | SEQ ID NO: 37 |
| 5′-RET(ex1-11 NM_020630)- | 43611801/ | NM_014800 | SEQ ID NO: 103 |
| ELMO1(ex14-22 NM_014800) | chr7: 37238470- | | |
| Breakpoints RET intron 11, ELMO1 | 37238795 | | |
| intron 13 | RET ex1-11 | | |
| Lung adenocarcinoma | ELMO1 ex14-22 | | |
| In frame fusion. | | | |
| RET - WDFY4 | chr10: 43615421- | NM_020630/ | SEQ ID NO: 38 |
| 5′-RET(ex1-15 NM_020630)- | 43615713/ | NM_020945 | SEQ ID NO: 104 |
| WDFY4(ex40-62 NM_020945) | chr10: 50051602- | | |
| Breakpoints RET intron 15, WDFY4 | 50051938 | | |
| intron 39 | RET ex1-15 | | |
| Esophagus adenocarcinoma | WDFY4 ex40-62 | | |
| In frame fusion. | | | |
| MKX - RET | chr10: 27975034- | NM_173576/ | |
| 5′-MKX(ex1-5 NM_173576)- | 27975183/ | NM_020630 | |
| RET(ex12-19 NM_020630) | chr10: 43610285- | | |
| Breakpoints MKX intron 5, RET intron | 43610466 | | |
| 11 | MKX ex1-5 | | |
| Lung adenocarcinoma | RET ex12-19 | | |
| Includes RET kinase domain. | | | |
| RBMS3 -RET | chr3: 29823809- | NM_014483/ | |
| 5′-RBMS3(ex1-6 NM_014483)- | 29823976/ | NM_020630 | |
| RET(ex12-19 NM_020630) | chr10: 43612021- | | |
| Breakpoints RBMS3 intron 6, RET | 43612151 | | |
| intron 11 | RBMS3 ex1-6 | | |
| Uterus endometrial adenocarcinoma | RET ex12-19 | | |
| (NOS) | | | |
| Includes RET kinase domain. | | | |
| SGIP1 - RET | chr1: 67073312- | NM_032291/ | |
| 5′-SGIP1(ex1-1 NM_032291)- | 67073582/ | NM_020630 | |
| RET(ex12-19 NM_020630) | chr10: 43611993- | | |
| Breakpoints SGIP1 intron 1, RET intron | 43612185 | | |
| 11 | SGIP1 ex1-1 | | |
| Lung adenocarcinoma | RET ex12-19 | | |
| Includes RET kinase domain. | | | |
| ZSWIM6 - RET | chr5: 60644190- | NM_020928/ | |
| 5′-ZSWIM6(ex1-1 NM_020928)- | 60644600/ | NM_020630 | |
| RET(ex12-19 NM_020630) | chr10: 43611931- | | |
| Breakpoints ZSWIM6 intron 1, RET | 43612183 | | |
| intron 11 | ZSWIM6 ex1-1 | | |
| Lung adenocarcinoma | RET ex12-19 | | |
| Includes RET kinase domain. | | | |
| ALOX5 - RET | chr10: 45880054- | NM_000698/ | |
| 5′-ALOX5(ex1-2 NM_000698)- | 45880377/ | NM_020630 | |
| RET(ex12-19 NM_020630) | chr10: 43611435- | | |
| Breakpoints ALOX5 intron 2, RET | 43611795 | | |
| intron 11 | ALOX5 ex1-2 | | |
| Brain glioblastoma (GBM) | RET ex12-19 | | |
| Includes RET kinase domain. | | | |
| RET - NTRK2 | chr10: 43609278- | NM_020630/ | |
| 5′-RET(ex1-10 NM_020630)- | 43609516/ | NM_006180 | |
| NTRK2(ex20-21 NM_006180) | chr9: 87615294- | | |
| Breakpoints RET intron 10, NTRK2 | 87615609 | | |
| intron 19 | RET ex1-10 | | |
| Bladder urothelial (transitional cell) | NTRK2 ex20-21 | | |
| carcinoma | | | |
| RET - OXR1 | chr10: 43611090- | NM_020630/ | |
| 5′-RET(ex1-11 NM_020630)- | 43611446/ | NM_018002 | |
| OXR1(ex4-16 NM_018002) | chr8: 107581829- | | |
| Breakpoints RET intron 11, OXR1 | 107582082 | | |
| intron 3 | RET ex1-11 | | |
| Unknown primary serous carcinoma | OXR1 ex4-16 | | |
| RET - CCBE1 | chr10: 43607774- | NM_020630/ | |
| 5′-RET(ex1-8 NM_020630)- | 43607874/ | NM_133459 | |
| CCBE1(ex3-11 NM_133459) | chr18: 57343203- | | |
| Breakpoints RET intron 8, CCBE1 | 57343676 | | |
| intron 2 | RET ex1-8 | | |
| Lung adenocarcinoma | CCBE1 ex3-11 | | |
| RET - NAALADL2 | chr10: 43611602- | NM_020630/ | |
| 5′-RET(ex1-11 NM_020630)- | 43611922/ | NM_207015 | |
| NAALADL2(ex13-14 NM_207015) | chr3: 175458489- | | |
| Breakpoints RET intron 11, | 175458778 | | |
| NAALADL2 intron 12 | RET ex1-11 | | |
| Thyroid papillary carcinoma | NAALADL2 ex13-14 | | |
| RAI14- RET | chr5: 34825746- | NM_015577/ | |
| 5′-RAI14(ex1-15 NM_015577)- | 34825976/ | NM_020630 | |
| RET(ex11-19 NM_020630) | chr10: 43610042- | | |
| Breakpoints RAI14 intron 15, RET | 43610268 | | |
| exon 11 | RAI14 ex1-15 | | |
| Lung adenocarcinoma | RET ex11-19 | | |
| Includes RET kinase domain | | | |
| ABI3BP - RET | chr3: 100469166- | NM_015429/ | |
| 5′-ABI3BP(ex1-35 NM_015429)- | 100469527/ | NM_020630 | |
| RET(ex12-19 NM_020630) | chr10: 43610324- | | |
| Breakpoints ABI3BP exon 35, RET | 43610920 | | |
| intron 11 | ABI3BP ex1-35 | | |
| Thyroid carcinoma (NOS) | RET ex12-19 | | |
| Includes RET kinase domain | | | |
| LINC00379 - RET | chr13: 91833445- | NR_047004/ | |
| 5′-LINC00379(ex1-2 NR_047004)- | 91833706/ | NM_020630 | |
| RET(ex12-19 NM_020630) | chr10: 43611105- | | |
| Breakpoints LINC00379 intron 2, RET | 43611505 | | |
| intron 11 | LINC00379 ex1-2 | | |
| Breast carcinoma (NOS) | RET ex12-19 | | |
| Includes RET kinase domain | | | |
| OPTN - RET | chr10: 13145247- | NM_021980/ | |
| 5′-OPTN(ex1-1 NM_021980)- | 13145480/ | NM_020630 | |
| RET(ex11-19 NM_020630) | chr10: 43609677- | | |
| Breakpoints OPTN intron 1, RET intron | 43609931 | | |
| 10 | OPTN ex1-1 | | |
| Lung adenocarcinoma | RET ex11-19 | | |
| Includes RET kinase domain | | | |
| SH2D3A - RET | chr19: 6765967- | NM_005490/ | |
| 5′-SH2D3A(ex1-1 NM_005490)- | 6766439/ | NM_020630 | |
| RET(ex12-19 NM_020630) | chr10: 43611764- | | |
| Breakpoints SH2D3A intron 1, RET | 43612170 | | |
| intron 11 | SH2D3A ex1-1 | | |
| Lung non-small cell lung carcinoma | RET ex12-19 | | |
| (NOS) | | | |
| Includes RET kinase domain | | | |
| ZFN721 - RET | chr4: 472119-472287/ | NM_133474/ | |
| 5′-(ZNF721 ex1-1 NM_133474)-(RET | chr10: 43610406- | NM_020630 | |
| ex12-19 NM_020630) Breakpoints | 43610589 | | |
| ZNF721 intron 1, RET intron 11 | ZNF721 ex1-1 | | |
| Lung non-small cell lung carcinoma | RET ex12-19 | | |
| (NOS) | | | |
| Includes RET kinase domain | | | |
| ADAMTS14 - RET | chr10: 72480354/ | NM_080722/ | |
| 5′-ADAMTS14(ex1-4 NM_080722)- | chr10: 43611720 | NM_020630 | |
| RET(ex12-19 NM_020630) | ADAMTS14 ex1-4 | | |
| Breakpoints ADAMTS14 intron 4, RET | RET ex12-19 | | |
| intron 11 | | | |
| Esophagus adenocarcinoma | | | |
| Includes RET kinase domain | | | |
| CSGALNACT2 - RET | chr10: 43648115- | NM_018590/ | |
| 5′-CSGALNACT2(ex1-1 NM_018590)- | 43648300/ | NM_020630 | |
| RET(ex12-19 NM_020630) | chr10: 43610689- | | |
| Breakpoints CSGALNACT2 intron 1, | 43610896 | | |
| RET intron 11 | CSGALNACT2 ex1-1 | | |
| Uterus carcinosarcoma | RET ex 12-19 | | |
| Includes RET kinase domain | | | |
| CPEB3 - RET | chr10: 93808581- | NM_014912/ | |
| 5′-CPEB3(ex1-10 NM_014912)- | 93808772/ | NM_020630 | |
| RET(ex12-19 NM_020630) | chr10: 43611585- | | |
| Breakpoints CPEB3 exon 10, RET | 43611862 | | |
| intron 11 | CPEB3 ex1-10 | | |
| Lung adenocarcinoma | RET ex12-19 | | |
| Includes RET kinase domain | | | |
| RET - CCNY | chr10: 43611301- | NM_020630/ | |
| 5′-RET(ex1-11 NM_020630)- | 43611576/ | NM_181698 | |
| CCNY(ex4-12 NM_181698) | chr10: 35549015- | | |
| Breakpoints RET intron 11, CCNY | 35549283 | | |
| intron 3 | RET ex1-11 | | |
| Breast invasive ductal carcinoma (IDC) | CCNY ex4-12 | | |
| RET - RASGEF1A | chr10: 43601756- | NM_020630/ | |
| 5′-RET(ex1-5 NM_020630)- | 43602031/ | NM_145313 | |
| RASGEF1A(ex2-13 NM_145313) | chr10: 43709383- | | |
| Breakpoints RET intron 5, RASGEF1A | 43709498 | | |
| intron 1 | RET ex1-5 | | |
| Unknown primary melanoma | RASGEF1A ex2-13 | | |
| RET - HSD17B7P2 | chr10: 43609878- | NM_020630/ | |
| 5′-RET(ex1-11 NM_020630)- | 43609957/ | NR_003086 | |
| HSD17B7P2(ex8-8 NR_003086) | chr10: 38667312- | | |
| Breakpoints HSD17B7P2 exon 8, RET | 38667485 | | |
| exon 11 | RET ex1-11 | | |
| Unknown primary carcinoma (NOS) | HSD17B7P2 ex8-8 | | |
| RET - RAD1 | chr10: 43609716- | NM_020630/ | |
| 5′-RET(ex1-11 NM_020630)- | 43610036/ | NM_002853 | |
| RAD1(ex6-6 NM_002853) Breakpoints | chr5: 34906366- | | |
| RET exon 11, RAD1 exon 6 | 34906711 | | |
| Lung adenocarcinoma | RET ex1-11 | | |
| | RAD1 ex6-6 | | |
| RET - VSTM4 | chr10: 43609237- | NM_020630/ | |
| 5′-RET(ex1-10 NM_020630)- | 43609437/ | NM_001031746 | |
| VSTM4(ex8-8 NM_001031746) | chr10: 50225719- | | |
| Breakpoints RET intron 10, VSTM4 | 50225999 | | |
| exon 8. | RET ex1-10 | | |
| Bladder urothelial (transitional cell) | VSTM4 ex8-8 | | |
| carcinoma | | | |

### Example 2: Clinicopathologic and Genomic Landscape of Solid Tumors with RET Fusions

This Example describes the clinicopathologic and genomic profiles of 891 patients with RET fusion-driven advanced solid tumors.

### Methods

### Tissue DNA Sequencing Assay

Genomic profiling was performed on patient samples using a tissue-based DNA hybrid-capture next generation sequencing (NGS) assay. The tissue-based NGS assay used a hybrid capture methodology to detect base substitutions, insertion and deletion alterations (indels), and copy number alterations (CNAs) in up to 324 genes and select gene rearrangements (Frampton, G. M., et al., Nat Biotechnol, 2013. 31 (11): p. 1023-31). Each sample's hematoxylin and eosin staining was reviewed under light microscopy to determine suitability for genomic profiling (at least 20% tumor nuclei present), and to confirm the diagnosis of the sample.

### Tumor Mutational Burden, Microsatellite Instability, Mutational Signatures

Tumor mutational burden (TMB) was determined on 0.8-1.1 Mb of sequenced DNA, and microsatellite instability (MSI) was assessed by DNA sequencing across 114 loci. See, Rhoden, K. J., et al., J Clin Endocrinol Metab, 2006. 91 (6): p. 2414-23, Chalmers, Z. R., et al., Genome Med, 2017. 9 (1): p. 34, and Trabucco, S. E., et al., J Mol Diagn, 2019. 21 (6): p. 1053-1066. TMB-high (H) was defined as ≥10 mutations/Megabase.

Mutational signatures and genetic ancestry were also assessed. Specifically, tobacco mutational signatures were called as previously described (Zehir, A., et al., Nat Med, 2017. 23 (6): p. 703-713). Genetic ancestry was assessed using a SNP-based approach, and patients were estimated to be of predominately African, European, Central and South American, South Asian, or East Asian genetic ancestry (Connelly, C. F., et al., Abstract 1227, 2018. 78 (13 Supplement): p. 1227-1227).

### Liquid DNA Sequencing Assay

A subset of the samples were tested with liquid biopsies utilizing a liquid-based hybrid capture NGS assay. The liquid-based NGS assay used a hybrid capture methodology to detect base substitutions, insertion and deletion alterations (indels), and copy number alterations (CNAs) in up to 324 genes and select gene rearrangements (Woodhouse, R., et al., PLOS One, 2020. 15 (9): p. e0237802). The levels of circulating tumor DNA (ctDNA) shed for each specimen were quantified by calculation of the composite tumor fraction (cTF), which merges two methods for estimation of tumor fraction (TF). When TF is elevated (generally above 10%), an estimate is returned based on a measure of tumor aneuploidy that incorporates observed deviations in coverage across the genome. See, Li, G., et al., J Gastrointest Oncol, 2019. 10 (5): p. 831-840, Tukachinsky, H., et al., Clin Cancer Res, 2021. 27 (11): p. 3094-3105., Li M, F. B., Yang L et al, Cancer Res, 2021. 81: p. 2231-2231. This aneuploidy-based approach avoided erroneous inferences of elevated TF due to the presence of germline variants detected at high variant allele frequency. When lack of tumor aneuploidy limited the ability to estimate TF (generally at lower TF), a variant-based calculation was made by identification of the highest allele fraction non-germline variant, excluding specific clonal hematopoiesis (CH) associated alterations.

### RET Fusion Case Selection

All RET fusions as detected by the tissue-based and liquid-based NGS assays were included in this study. Rearrangements without a fusion partner were excluded from the analysis. Only cases where the kinase domain of RET was preserved were included in the study.

### PD-LI Expression

Programmed death-ligand 1 (PD-L1) expression was assessed using a DAKO PD-L1 22C3 immunohistochemistry (IHC) assay, performed concurrently with the tissue-based NGS assay for a subset of samples (236 cases). Staining was evaluated using the tumor proportion score (TPS) method, where TPS=#PD-L1 positive tumor cells/(Total # of PD-L1 positive+PD-L1 negative tumor cells), as previously described (DAKO. PD-L1 IHC 22C3 pharmDx Interpretation Manual-NSCLC).

### Data Analysis

The Fisher's Exact Test or Chi-squared Test for categorical variables, and the Wilcox Rank Sum test for continuous variables, were used to assess the clinical, pathologic, biomarker, and genomic differences between the different cohorts in this study. P-values were adjusted for multiple comparisons using the Bonferroni method, and p<0.05 was considered significant (Goeman, J. J, and A. Solari, Multiple hypothesis testing in genomics. 2014. 33 (11): p. 1946-1978).

### Results

### Clinicopathologic Characteristics

A characterization of the clinicopathologic and genomic characteristics of 891 patients with RET fusions in different cancer types was undertaken (523 patients with non-small cell lung cancer [NSCLC] and 368 patients with other, non-NSCLC solid tumors). See, Table 14.

TABLE 14
Comparison of clinicopathologic and genomic biomarker characteristics
of RET fusion-positive NSCLC cases and RET fusion-positive
cases in other solid tumors (non-NSCLC).

| | RET fusion+ NSCLC | | RET fusion+ non-NSCLC | | |
|---|---|---|---|---|---|
| | (n = 523) | | (n = 368) | | p value |
| Age median years [IQR]* | 64 | [55-71] | 61 | [42-69] | <0.001 |
| Sex, Female/Male (%) | 293/230 | (56%/44%) | 211/157 | (57%/43%) | 0.731 |
| Specimen Site | | | | | |
| Primary | 249 | (48%) | 163 | (44%) | 0.623 |
| Metastatic | 274 | (52%) | 155 | (42%) | 0.140 |
| Cancers of unknown primary | 0 | | 38 | (10%) | n/a |
| Unknown | 0 | (0%) | 12 | (3.3) | <0.001 |
| Ancestry** | | | | | |
| African | 41 | (7.8%) | 35 | (9.5%) | 1 |
| Central and South American | 45 | (8.6%) | 58 | (16%) | 0.007 |
| East Asian | 55 | (11%) | 14 | (3.8%) | 0.001 |
| European | 372 | (71%) | 254 | (69%) | 1 |
| South Asian | 10 | (1.9%) | 7 | (1.9%) | 1 |
| unknown | 0 | (0%) | 0 | (0%) | 1 |
| Tobacco mutational signature | 3 | (0.6%) | 1 | (0.3%) | 0.647 |
| ICPI Biomarkers | | | | | |
| PD-L1 (DAKO 22C3)*** | n = 141 | | n = 95 | | <0.001 |
| TPS <1 | 31 | (22%) | 57 | (60%) | |
| TPS 1-49 | 59 | (42%) | 24 | (25%) | |
| TPS >=50 | 51 | (36%) | 14 | (15%) | |
| TMB-H | 27 | (5.2%) | 45 | (12%) | <0.001 |
| TMB median mut/Mb* | 1.8 | [0.9-3.8] | 1.7 | [0-5.0] | 0.741 |
| MSI-H | 0 | (0%) | 29 | (7.9%) | <0.001 |

Tumor Proportion Score (TPS). Tumor mutational burden-High (TMB-H). Microsatellite instability-high (MSI-H).
*Wilcox Rank Sum Test.
**p-value adjusted for multiple comparisons.
***Chi-squared Test. IQR = interquartile range.

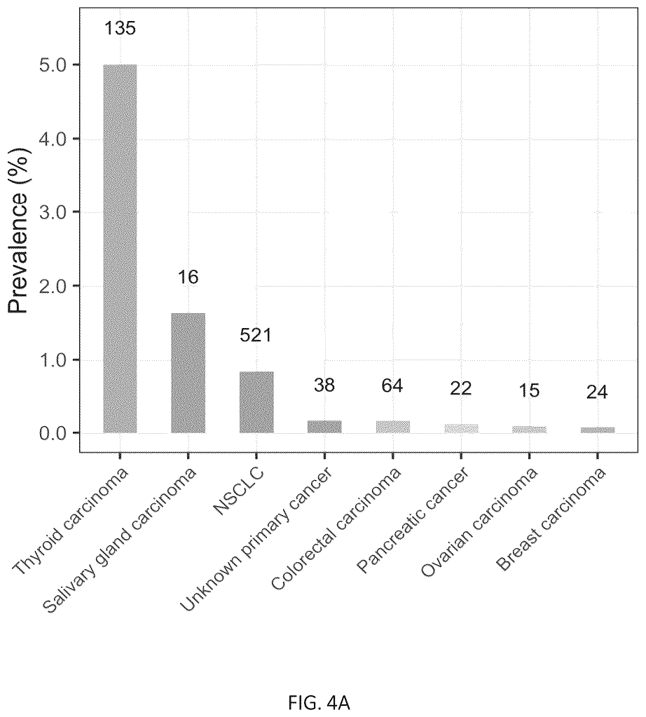

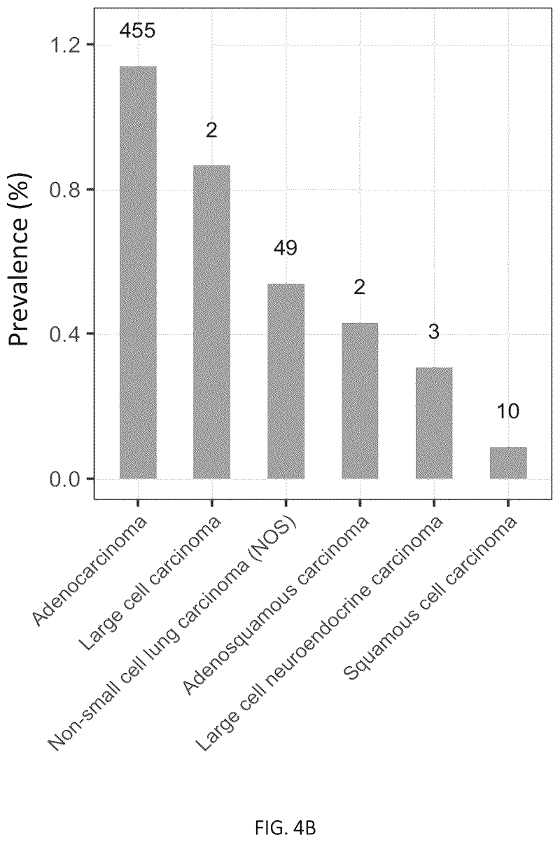

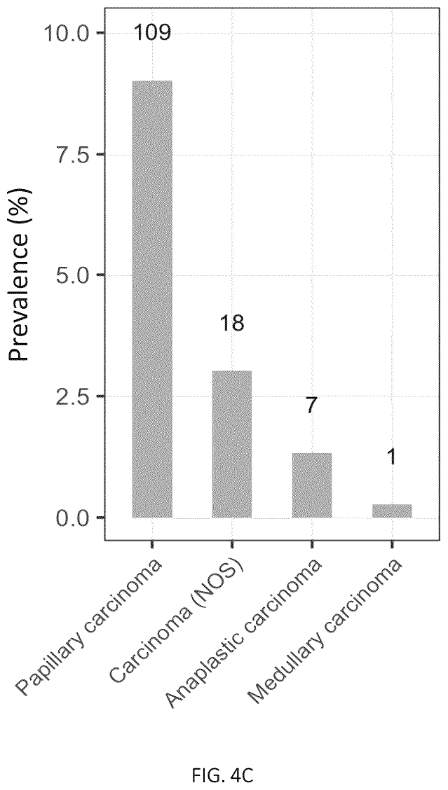

The prevalence of RET fusions varied based on tumor type. See, FIGS. 4A-4C. The two tumor types with the highest number of RET fusions were lung adenocarcinoma and thyroid papillary carcinoma, which had prevalence rates of 1.14% and 9.02%, respectively. The non-NSCLC cohort consisted of mainly thyroid carcinomas (36.6%, 135/368) and gastrointestinal colorectal carcinomas (17.3%, 64/368), followed by carcinomas of unknown primary (10.3%, 38/368), breast carcinomas (6.5%, 24/368), pancreatic carcinomas (5.9%, 22/368), and a wide range of other tumor types. See, Tables 15 and 16. Among the several histologic subtypes, thyroid papillary carcinoma, colon adenocarcinoma, breast and pancreatic ductal carcinoma, and intra-hepatic cholangiocarcinoma were the most frequent cancers with relatively high prevalence of RET fusion positivity.

TABLE 15
Frequency of RET fusion-positive advanced
NSCLC and other solid tumors (non-NSCLC).

| | | RET fusion |
|---|---|---|
| Diagnosis | Prevalence | cases |
| Lung adenocarcinoma | 1.14% | 455 |
| Thyroid papillary carcinoma | 9.02% | 109 |
| Colon adenocarcinoma | 0.18% | 60 |
| Lung non-small cell lung carcinoma (NOS) | 0.54% | 49 |
| Thyroid carcinoma (NOS) | 3.02% | 18 |
| Pancreas ductal adenocarcinoma | 0.11% | 17 |
| Unknown primary adenocarcinoma | 0.19% | 16 |
| Unknown primary carcinoma (CUP) | 0.31% | 14 |
| Breast carcinoma (NOS) | 0.06% | 12 |
| Breast invasive ductal carcinoma | 0.09% | 11 |
| Intra-hepatic cholangiocarcinoma | 0.15% | 9 |
| Lung squamous cell carcinoma | 0.08% | 9 |
| Salivary gland carcinoma (NOS) | 1.79% | 8 |
| Brain glioblastoma | 0.12% | 8 |
| Thyroid anaplastic carcinoma | 1.33% | 7 |
| Ovary epithelial carcinoma | 0.16% | 5 |
| Ovary serous carcinoma | 0.05% | 5 |
| Prostate acinar adenocarcinoma | 0.04% | 5 |
| Salivary gland adenocarcinoma | 1.24% | 4 |
| Esophagus adenocarcinoma | 0.08% | 4 |
| Bladder urothelial carcinoma | 0.07% | 4 |
| Salivary gland duct carcinoma | 1.46% | 3 |
| Colon neuroendocrine carcinoma | 0.72% | 3 |
| Lung sarcomatoid carcinoma | 0.70% | 3 |
| Small intestine adenocarcinoma | 0.37% | 3 |
| Lung large cell neuroendocrine carcinoma | 0.31% | 3 |
| Ovary high grade serous carcinoma | 0.14% | 3 |
| Pancreatobiliary carcinoma | 0.11% | 3 |
| Lung small cell undifferentiated carcinoma | 0.09% | 3 |
| Stomach adenocarcinoma (NOS) | 0.06% | 3 |
| Lung atypical carcinoid | 0.92% | 2 |
| Lung large cell carcinoma | 0.87% | 2 |
| Lung adenosquamous carcinoma | 0.43% | 2 |
| Pancreas islet cell tumor | 0.22% | 2 |
| Pancreas carcinoma (NOS) | 0.10% | 2 |
| Unknown primary melanoma | 0.05% | 2 |
| Salivary gland mammary analogue secretory | 11.11% | 1 |
| carcinoma | | |
| Adrenal gland pheochromocytoma | 1.37% | 1 |
| Brain Gliosarcoma | 0.85% | 1 |
| Soft tissue leiomyosarcoma | 0.78% | 1 |
| Pancreas acinar cell carcinoma | 0.54% | 1 |
| Skin adnexal carcinoma | 0.48% | 1 |
| Uterus endometrial adenocarcinoma mixed | 0.44% | 1 |
| histology | | |
| Esophagus carcinoma (NOS) | 0.28% | 1 |
| Thyroid medullary carcinoma | 0.27% | 1 |
| Unknown primary neuroendocrine tumor | 0.19% | 1 |
| Ovary carcinosarcoma | 0.17% | 1 |
| Unknown primary serous carcinoma | 0.16% | 1 |
| Unknown primary (NOS) | 0.13% | 1 |
| Unknown primary urothelial carcinoma | 0.12% | 1 |
| Extra-hepatic cholangiocarcinoma | 0.11% | 1 |
| Brain meningioma | 0.11% | 1 |
| Skin squamous cell carcinoma | 0.10% | 1 |
| Uterus carcinosarcoma | 0.06% | 1 |
| Unknown primary undifferentiated | 0.06% | 1 |
| neuroendocrine carcinoma | | |
| Breast invasive lobular carcinoma | 0.05% | 1 |
| Unknown primary squamous cell carcinoma | 0.05% | 1 |
| Uterus endometrial adenocarcinoma (NOS) | 0.02% | 1 |
| Rectum adenocarcinoma | 0.01% | 1 |
| Total | | 891 |

TABLE 16
RET fusion-positive other solid tumors (non-
NSCLC) and respective diagnostic subtypes.

| Tumor type | n |
|---|---|
| Thyroid carcinoma | 135 |
| Thyroid anaplastic carcinoma | 7 |
| Thyroid carcinoma (NOS) | 18 |
| Thyroid medullary carcinoma | 1 |
| Thyroid papillary carcinoma | 109 |
| Colorectal carcinoma | 64 |
| Colon adenocarcinoma (CRC) | 60 |
| Colon neuroendocrine carcinoma | 3 |
| Rectum adenocarcinoma (CRC) | 1 |
| Unknown primary cancer | 38 |
| Unknown primary (NOS) | 1 |
| Unknown primary adenocarcinoma | 16 |
| Unknown primary carcinoma (cup) (NOS) | 14 |
| Unknown primary melanoma | 2 |
| Unknown primary neuroendocrine tumor | 1 |
| Unknown primary serous carcinoma | 1 |
| Unknown primary squamous cell carcinoma (SCC) | 1 |
| Unknown primary undifferentiated neuroendocrine carcinoma | 1 |
| Unknown primary urothelial carcinoma | 1 |
| Breast carcinoma | 24 |
| Breast carcinoma (NOS) | 12 |
| Breast invasive ductal carcinoma (IDC) | 11 |
| Breast invasive lobular carcinoma (ILC) | 1 |
| Pancreatic cancer | 22 |
| Pancreas acinar cell carcinoma | 1 |
| Pancreas carcinoma (NOS) | 2 |
| Pancreas ductal adenocarcinoma | 17 |
| Pancreas islet cell tumor | 2 |
| Salivary gland carcinoma | 16 |
| Salivary gland adenocarcinoma | 4 |
| Salivary gland carcinoma (NOS) | 8 |
| Salivary gland duct carcinoma | 3 |
| Salivary gland mammary analogue secretory carcinoma (masc) | 1 |
| Ovarian carcinoma | 14 |
| Ovary carcinosarcoma | 1 |
| Ovary epithelial carcinoma | 5 |
| Ovary high grade serous carcinoma | 3 |
| Ovary serous carcinoma | 5 |
| Cholangiocarcinoma | 10 |
| Extra-hepatic cholangiocarcinoma | 1 |
| Intra-hepatic cholangiocarcinoma | 9 |
| Brain cancer | 10 |
| Brain glioblastoma (GBM) | 8 |
| Brain gliosarcoma | 1 |
| Brain meningioma | 1 |
| Lung cancer (non-NSCLC) | 5 |
| Lung atypical carcinoid | 2 |
| Lung small cell undifferentiated carcinoma | 3 |
| Prostate acinar adenocarcinoma | 5 |
| Esophageal carcinoma | 5 |
| Esophagus adenocarcinoma | 4 |
| Esophagus carcinoma (NOS) | 1 |
| Bladder urothelial (transitional cell) carcinoma | 4 |
| Uterine carcinoma | 3 |
| Uterus carcinosarcoma | 1 |
| Uterus endometrial adenocarcinoma (NOS) | 1 |
| Uterus endometrial adenocarcinoma mixed histology | 1 |
| Stomach adenocarcinoma (NOS) | 3 |
| Small intestine adenocarcinoma | 3 |
| Pancreatobiliary carcinoma | 3 |
| Skin carcinoma | 2 |
| Skin adnexal carcinoma | 1 |
| Skin squamous cell carcinoma (SCC) | 1 |
| Adrenal gland pheochromocytoma | 1 |
| Soft tissue leiomyosarcoma | 1 |
| Total | 368 |

The NSCLC RET fusion-positive cohort was significantly younger (median age=64 vs. 68; P <0.001), had a higher female: male ratio (1.27 vs. 1.02; P=0.012), and had a higher frequency of specimens obtained from metastatic sites versus nonmetastatic sites (52% vs. 43%; P=0.002) when compared to the NSCLC RET fusion-negative cohort. See, Table 17. In the NSCLC cohort, patients with Central and South American, East Asian, and South Asian ancestry were more highly represented in the RET fusion-positive subset versus the RET fusion-negative subset (8.6% vs. 5.1%, 11% vs. 4.3%, and 1.9% vs. 0.6%, respectively; P<0.001). Lastly, there was a significant decrease in the tobacco smoking mutational signature in the NSCLC RET fusion-positive cohort when compared to the NSCLC RET fusion-negative cohort (0.6% vs. 13%; P<0.001).

TABLE 17
Clinicopathologic and genomic biomarker characteristics comparing
RET fusion-positive NSCLC and RET fusion-negative NSCLC.

| | RET fusion+ | | RET fusion− | | |
|---|---|---|---|---|---|
| | NSCLC | | NSCLC | | |
| | (n = 523) | | (n = 61,310) | | p value |
| Age median years | 64 | [55-71] | 68 | [60-75] | <0.001 |
| [IQR]* | | | | | |
| Sex, Female/Male (%) | 293/230 | | 30,900/30,410 | | 0.012 |
| | (56%/44%) | | (50%/50%) | | |
| Specimen Site | | | | | |
| Primary | 249 | (48%) | 34,981 | (57%) | <0.001 |
| Metastatic | 274 | (52%) | 26203 | (43%) | 0.002 |
| Unknown | 0 | (0%) | 126 | (0.2%) | 0.304 |
| Ancestry** | | | | | |
| African | 41 | (7.8%) | 5882 | (9.6%) | 0.200 |
| Central and South | 45 | (8.6%) | 3148 | (5.1%) | <0.001 |
| American | | | | | |
| East Asian | 55 | (11%) | 2648 | (4.3%) | <0.001 |
| European | 372 | (71%) | 49,232 | (80.3%) | <0.001 |
| South Asian | 10 | (1.9%) | 389 | (0.6%) | 0.001 |
| unknown | 0 | (0%) | 11 | (<0.1%) | 0.759 |
| Tobacco mutational | 3 | (0.6%) | 7,776 | (13%) | <0.001 |
| signature | | | | | |
| ICPI Biomarkers | | | | | |
| PD-L1 (DAKO | n = 141 | | n = 23,640 | | <0.001 |
| 22C3)*** | | | | | |
| TPS <1 | 31 | (22%) | 9026 | (38%) | |
| TPS 1-49 | 59 | (42%) | 7088 | (30%) | |
| TPS >=50 | 51 | (36%) | 7526 | (32%) | |
| TMB-H | 27 | (5.2%) | 21,896 | (36%) | <0.001 |
| TMB median | 1.8 | [0.9-3.8] | 7.0 | [2.6-12.5] | <0.001 |
| mut/Mb* | | | | | |
| MSI-H | 0 | (0%) | 219 | (0.4%) | 0.318 |

*Wilcox Rank Sum Test;
**p-value adjusted for multiple comparisons;
***Chi-squared Test; IQR, interquartile range.

For the non-NSCLC RET fusion-positive cohort, the NSCLC RET fusion-positive cohort was used for inter-cohort comparison. The non-NSCLC RET fusion-positive patients (368) were significantly younger compared to NSCLC RET fusion-positive cases (median age=61 vs. 64; P <0.001). In comparison to the NSCLC RET fusion-positive cohort, the non-NSCLC RET fusion-positive cohort had a significantly higher prevalence of Central and South American and East Asian ancestry (8.6% vs. 16%, 11% vs. 3.8%, respectively; P<0.01). See, Table 14.

### RET In-Frame Fusion Partners and Breakpoints in NSCLC Versus Non-NSCLC

All the RET fusion events in this cohort of patients were in-frame events. Among all RET (10q11.21) fusion gene partners, 93% of genes resided in chromosome 10 across arms p and q. The top fusions partners identified in the NSCLC cohort were KIF5B (chr10 p11.22; 66%), CCDC6 (chr10 q21.2; 18.2%), NCOA4 (chr10 q11.23; 2.9%), TRIM24 (chr7 q34; 2%), ERC1 (chr12 p13.33; 1%), and KIAA1468 (chr18 q21.33; 1%). See, Table 18.

On the other hand, more than half of the non-NSCLC cohort was composed of RET fusions with gene fusion partners NCOA4 (32.6%) and CCDC6 (29.9%). Of note, the most common fusions in papillary thyroid carcinoma were CCDC6-RET and NCOA4-RET (41.3% and 35.8%, respectively). KIFSB-RET fusions were highly specific for NSCLC compared to non-NSCLC solid tumors (66% vs. 6.3%; P<0.001). In contrast, NCOA4-RET (32.6% vs. 2.9%, P<0.001) and CCDC6-RET (30% vs. 18.2%; P=0.002) fusions were frequently seen among non-NSCLC solid tumors. See, Tables 18 and 19.

TABLE 18
Prevalence of RET fusions and partner genes among
RET fusion-positive NSCLC and non-NSCLC solid
tumors (fusions with at least 5 cases).

| | | | Other | | |
|---|---|---|---|---|---|
| | | | solid | | |
| | NSCLC | | tumors | | Corrected |
| Fusions | (n = 523) | n | (n = 368) | n | p-value |
| KIF5B-RET* | 66.0% | 346 | 6.3% | 23 | <0.001 |
| CCDC6-RET* | 18.2% | 95 | 29.9% | 110 | 0.002 |
| NCOA4-RET* | 2.9% | 15 | 32.6% | 121 | <0.001 |
| ERC1-RET | 1.0% | 5 | 2.7% | 10 | 0.705 |
| TRIM24-RET | 0.8% | 4 | 2.4% | 9 | 0.705 |
| TRIM33-RET | 0.6% | 3 | 1.1% | 4 | 0.767 |
| CSGALNACT2-RET | 0.0% | 0 | 1.6% | 6 | 0.135 |
| KIAA1217-RET | 0.2% | 1 | 1.4% | 5 | 0.705 |
| KIAA1468-RET | 0.8% | 5 | 0.5% | 2 | 1 |

*Significantly associated RET fusion gene partners among RET fusion-positive NSCLC and other solid tumors (non-NSCLC).

TABLE 19
Prevalence of RET fusions and partner genes among RET fusion-positive
NSCLC and non-NSCLC solid tumors (fusions with <5 cases).

| | | | Other solid | | |
|---|---|---|---|---|---|
| | NSCLC | | tumors | | Corrected |
| Fusions | (n = 523) | n | (n = 368) | n | p-value |
| EML4-RET | 0 | 0 | 0.011 | 4 | 0.533 |
| GOLGA5-RET | 0 | 0 | 0.011 | 4 | 0.533 |
| BMS1-RET | 0.006 | 3 | 0.003 | 1 | 0.983 |
| PCM1-RET | 0 | 0 | 0.008 | 3 | 0.705 |
| SNRNP70-RET | 0.002 | 1 | 0.005 | 2 | 0.883 |
| RASGEF1A-RET | 0.004 | 2 | 0.003 | 1 | 1 |
| ABI3BP-RET | 0 | 0 | 0.005 | 2 | 0.705 |
| DLG5-RET | 0 | 0 | 0.005 | 2 | 0.705 |
| ETV6-RET | 0 | 0 | 0.005 | 2 | 0.705 |
| TAF3-RET | 0 | 0 | 0.005 | 2 | 0.705 |
| ZNF33B-RET | 0 | 0 | 0.005 | 2 | 0.705 |
| WAC-RET | 0.002 | 1 | 0.003 | 1 | 1 |
| EEA1-RET | 0.002 | 1 | 0.003 | 1 | 1 |
| RUFY2-RET | 0.002 | 1 | 0.003 | 1 | 1 |
| TNIP1-RET | 0.002 | 1 | 0.003 | 1 | 1 |
| KIF13A-RET | 0.004 | 2 | 0 | 0 | 0.805 |
| LSM14A-RET | 0.004 | 2 | 0 | 0 | 0.805 |
| PARD3-RET | 0.004 | 2 | 0 | 0 | 0.805 |
| PCDH15-RET | 0.004 | 2 | 0 | 0 | 0.805 |
| ARHGAP19-RET | 0.002 | 1 | 0 | 0 | 1 |
| ACPP-RET | 0 | 0 | 0.003 | 1 | 0.705 |
| ADAMTS14-RET | 0 | 0 | 0.003 | 1 | 0.705 |
| ADK-RET | 0 | 0 | 0.003 | 1 | 0.705 |
| AGBL4-RET | 0 | 0 | 0.003 | 1 | 0.705 |
| ALOX5-RET | 0 | 0 | 0.003 | 1 | 0.705 |
| ANKRD26-RET | 0 | 0 | 0.003 | 1 | 0.705 |
| C10orf99-RET | 0 | 0 | 0.003 | 1 | 0.705 |
| CASC8-RET | 0 | 0 | 0.003 | 1 | 0.705 |
| CCDC186-RET | 0 | 0 | 0.003 | 1 | 0.705 |
| CCNY-RET | 0 | 0 | 0.003 | 1 | 0.705 |
| CEP135-RET | 0 | 0 | 0.003 | 1 | 0.705 |
| EBF1-RET | 0 | 0 | 0.003 | 1 | 0.705 |
| ETS1-RET | 0 | 0 | 0.003 | 1 | 0.705 |
| FGFR1OP-RET | 0 | 0 | 0.003 | 1 | 0.705 |
| FRMD4A-RET | 0 | 0 | 0.003 | 1 | 0.705 |
| GNA14-RET | 0 | 0 | 0.003 | 1 | 0.705 |
| GRM7-RET | 0 | 0 | 0.003 | 1 | 0.705 |
| HOOK1-RET | 0 | 0 | 0.003 | 1 | 0.705 |
| IFT74-RET | 0 | 0 | 0.003 | 1 | 0.705 |
| LINC00379-RET | 0 | 0 | 0.003 | 1 | 0.705 |
| LMNA-RET | 0 | 0 | 0.003 | 1 | 0.705 |
| LRMDA-RET | 0 | 0 | 0.003 | 1 | 0.705 |
| MGEA5-RET | 0 | 0 | 0.003 | 1 | 0.705 |
| MLPH-RET | 0 | 0 | 0.003 | 1 | 0.705 |
| MYH14-RET | 0 | 0 | 0.003 | 1 | 0.705 |
| NTRK2-RET | 0 | 0 | 0.003 | 1 | 0.705 |
| OLFM4-RET | 0 | 0 | 0.003 | 1 | 0.705 |
| OXR1-RET | 0 | 0 | 0.003 | 1 | 0.705 |
| PDE4D-RET | 0 | 0 | 0.003 | 1 | 0.705 |
| PRKAR1A-RET | 0 | 0 | 0.003 | 1 | 0.705 |
| PRKG1-RET | 0 | 0 | 0.003 | 1 | 0.705 |
| RASAL2-RET | 0 | 0 | 0.003 | 1 | 0.705 |
| RBMS3-RET | 0 | 0 | 0.003 | 1 | 0.705 |
| REEP3-RET | 0 | 0 | 0.003 | 1 | 0.705 |
| RRBP1-RET | 0 | 0 | 0.003 | 1 | 0.705 |
| SAMD4A-RET | 0 | 0 | 0.003 | 1 | 0.705 |
| SATB1-RET | 0 | 0 | 0.003 | 1 | 0.705 |
| SLC36A2-RET | 0 | 0 | 0.003 | 1 | 0.705 |
| SORBS1-RET | 0 | 0 | 0.003 | 1 | 0.705 |
| SPECC1L-RET | 0 | 0 | 0.003 | 1 | 0.705 |
| SPIDR-RET | 0 | 0 | 0.003 | 1 | 0.705 |
| SQSTM1-RET | 0 | 0 | 0.003 | 1 | 0.705 |
| TFG-RET | 0 | 0 | 0.003 | 1 | 0.705 |
| TNIP2-RET | 0 | 0 | 0.003 | 1 | 0.705 |
| TRIM27-RET | 0 | 0 | 0.003 | 1 | 0.705 |
| UPF2-RET | 0 | 0 | 0.003 | 1 | 0.705 |
| VSTM4-RET | 0 | 0 | 0.003 | 1 | 0.705 |
| ZNF485-RET | 0 | 0 | 0.003 | 1 | 0.705 |
| ZNF487-RET | 0 | 0 | 0.003 | 1 | 0.705 |
| ATRNL1-RET | 0.002 | 1 | 0 | 0 | 1 |
| CCDC88C-RET | 0.002 | 1 | 0 | 0 | 1 |
| CHAT-RET | 0.002 | 1 | 0 | 0 | 1 |
| CLIP1-RET | 0.002 | 1 | 0 | 0 | 1 |
| CNTN1-RET | 0.002 | 1 | 0 | 0 | 1 |
| CPEB3-RET | 0.002 | 2 | 0 | 0 | 0.805 |
| DCC-RET | 0.002 | 1 | 0 | 0 | 1 |
| DOCK1-RET | 0.002 | 1 | 0 | 0 | 1 |
| DSP-RET | 0.002 | 1 | 0 | 0 | 1 |
| ELMO1-RET | 0.002 | 1 | 0 | 0 | 1 |
| GAS2-RET | 0.002 | 1 | 0 | 0 | 1 |
| LCOR-RET | 0.002 | 1 | 0 | 0 | 1 |
| MGMT-RET | 0.002 | 1 | 0 | 0 | 1 |
| MKX-RET | 0.002 | 1 | 0 | 0 | 1 |
| MYH9-RET | 0.002 | 1 | 0 | 0 | 1 |
| NPAS3-RET | 0.002 | 1 | 0 | 0 | 1 |
| OPTN-RET | 0.002 | 1 | 0 | 0 | 1 |
| PHYHIPL-RET | 0.002 | 1 | 0 | 0 | 1 |
| PIBF1-RET | 0.002 | 1 | 0 | 0 | 1 |
| RBPMS-RET | 0.002 | 1 | 0 | 0 | 1 |
| SGIP1-RET | 0.002 | 1 | 0 | 0 | 1 |
| SH2D3A-RET | 0.002 | 1 | 0 | 0 | 1 |
| SLC12A2-RET | 0.002 | 1 | 0 | 0 | 1 |
| TPR-RET | 0.002 | 1 | 0 | 0 | 1 |
| TRIM67-RET | 0.002 | 1 | 0 | 0 | 1 |
| UBE2D1-RET | 0.002 | 1 | 0 | 0 | 1 |
| WWTR1-RET | 0.002 | 1 | 0 | 0 | 1 |
| ZNF239-RET | 0.002 | 1 | 0 | 0 | 1 |
| ZSWIM6-RET | 0.002 | 1 | 0 | 0 | 1 |
| RAI14-RET | 0.002 | 1 | 0 | 0 | 1 |
| RAD1-RET | 0.002 | 1 | 0 | 0 | 1 |
| CCBE1-RET | 0.002 | 1 | 0 | 0 | 1 |
| NAALADL2-RET | 0.002 | 0 | 0 | 1 | 0.705 |

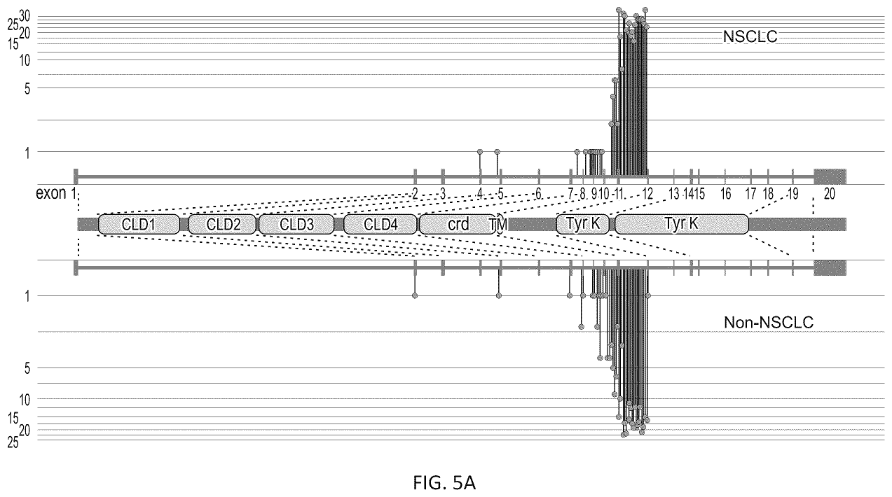

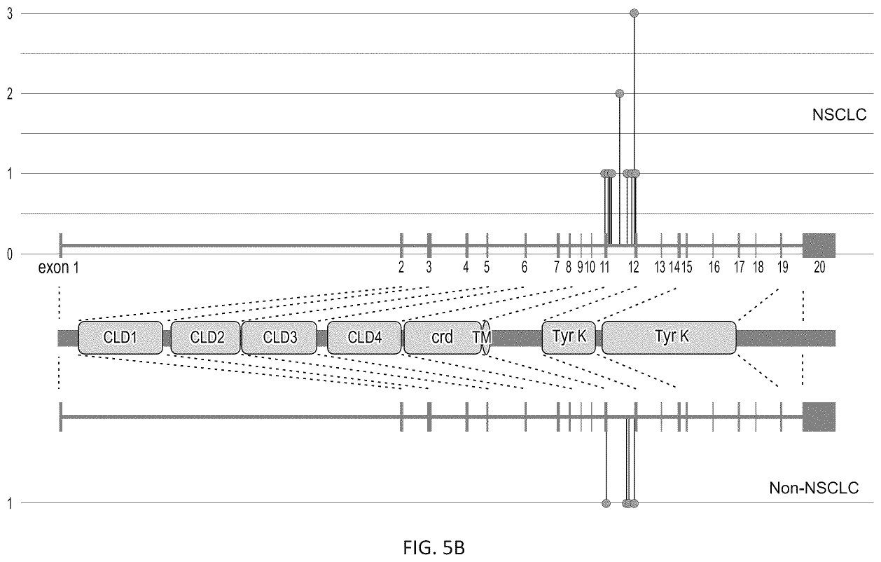

The primary breakpoint regions in RET were also examined. Among all the RET fusion-positive cohorts, the RET gene breakpoints were mainly clustered in intron 11 (87%) followed by intron 10 (5%) and exon 11 (4.8%). See, FIG. 5A. There was no significant difference in the distribution of RET breakpoint regions between NSCLC and non-NSCLC cases. Similarly, in liquid biopsies, RET gene breakpoints were mainly clustered in intron 11 in both the NSCLC and non-NSCLC RET fusion-positive cohorts. See, FIG. 5B.

Genes with Genomic Alterations in RET Fusion Defined Cohorts

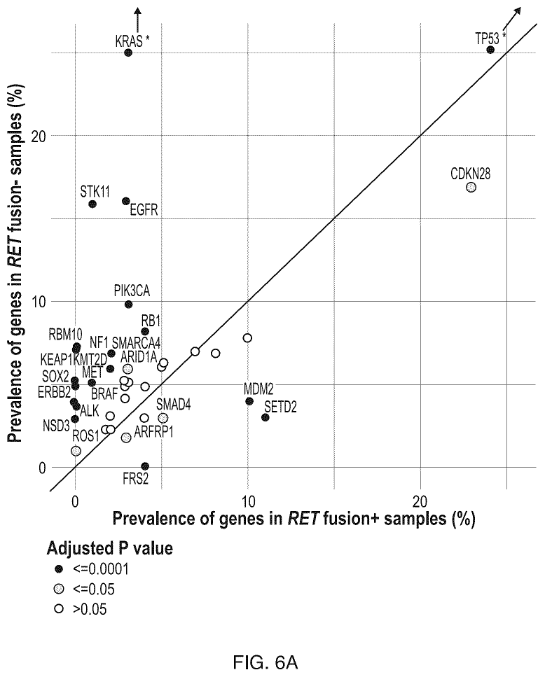

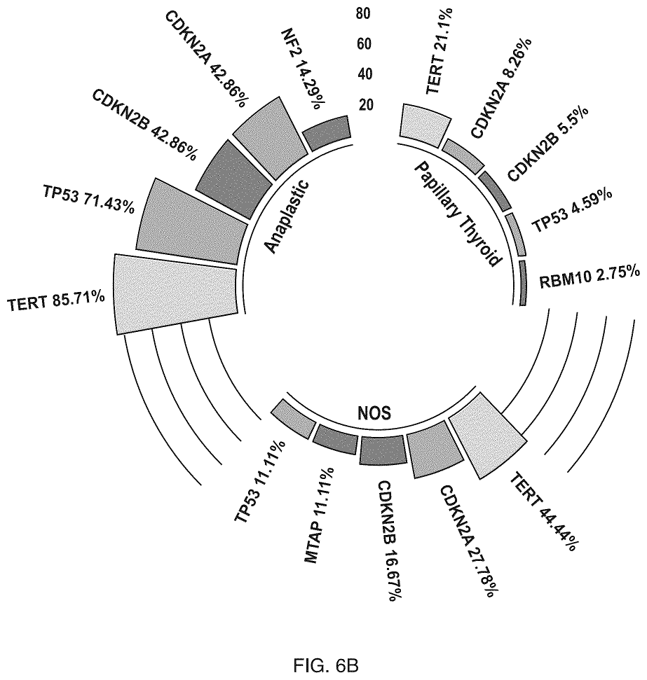

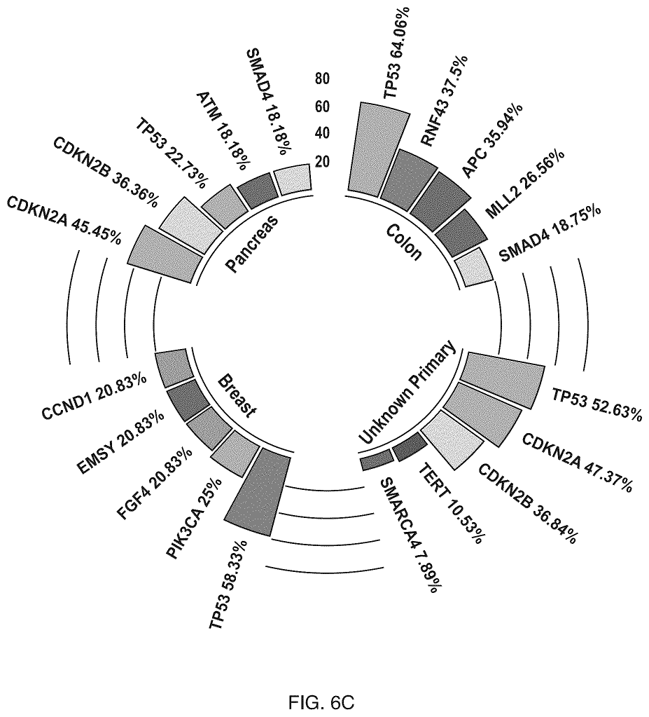

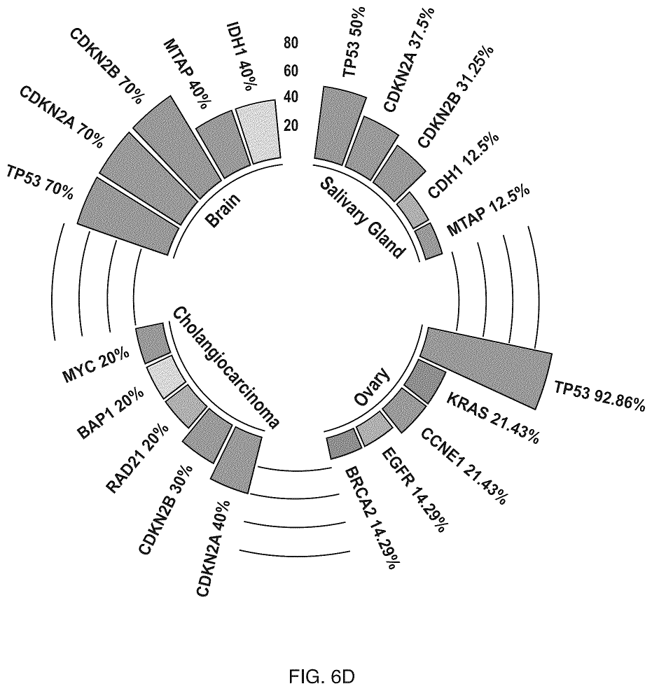

Frequently altered genes in RET fusion-positive and RET fusion-negative NSCLC cohorts were evaluated. The top 10 genes that were altered among RET fusion-positive NSCLC cases were TP53 (43%), CDKN2A (29%), CDKN2B (23%), SETD2 (11%), MDM2 (10%), MYC (10%), MTAP (8%), NKX2-1 (7%), NFKBIA (5%), and CDK4 (5%). In contrast, the top 10 genes that were altered among RET fusion-negative NSCLC patients were TP53 (68%), KRAS (31%), CDKN2A (29%), CDKN2B (17%), STK11 (16%), EGFR (16%), MTAP (13%), PIK3CA (10%), RB1 (8%), and MYC (8%). See, FIG. 6A. Significantly more common co-occurring gene alterations among RET fusion-positive versus negative NSCLC patients included CDKN2B, SETD2, MDM2, SMAD4, FRS2 and ARFRP1 (P<0.05). Similarly, significantly common co-occurring gene alterations among RET fusion-negative versus positive NSCLC patients included TP53, KRAS, STK11, EGFR, PIK3CA, RB1, NF1, SMARCA4, KEAP1, RBM10, ARID1A, KMT2D, SOX2, MET, BRAF, NSD3, ALK, ROS1, and ERBB2 (P<0.001). See, Table 20.

TABLE 20
Co-occurring genomic alterations and significant associations
among RET fusion-positive NSCLC and RET fusion-negative NSCLC.

| | RET fusion+ | | RET fusion− | | |
|---|---|---|---|---|---|
| | NSCLC | | NSCLC | | Corrected |
| Gene* | (n = 523) | % | (n = 61,310) | % | P-Value |
| TP53 | 223 | 43% | 41446 | 68% | <0.001 |
| KRAS | 14 | 3% | 18945 | 31% | <0.001 |
| CDKN2A | 153 | 29% | 17903 | 29% | 1 |
| CDKN2B | 120 | 23% | 10545 | 17% | 0.002 |
| STK11 | 5 | 1% | 9687 | 16% | <0.001 |
| EGFR | 17 | 3% | 9626 | 16% | <0.001 |
| MTAP | 40 | 8% | 4389 | 7% | 0.739 |
| PIK3CA | 14 | 3% | 6315 | 10% | <0.001 |
| RB1 | 19 | 4% | 5027 | 8% | <0.001 |
| MYC | 52 | 10% | 4843 | 8% | 0.118 |
| NF1 | 11 | 2% | 4476 | 7% | <0.001 |
| NKX2-1 | 37 | 7% | 4476 | 7% | 0.955 |
| SMARCA4 | 9 | 2% | 4353 | 7% | <0.001 |
| KEAP1 | 2 | 0% | 4292 | 7% | <0.001 |
| RBM10 | 2 | 0% | 4292 | 7% | <0.001 |
| ARID1A | 16 | 3% | 3924 | 6% | 0.002 |
| NFKBIA | 28 | 5% | 3679 | 6% | 0.728 |
| PTEN | 25 | 5% | 3556 | 6% | 0.473 |
| KMT2D | 8 | 2% | 3495 | 6% | <0.001 |
| SOX2 | 1 | 0% | 3311 | 5% | <0.001 |
| MET | 2 | 0% | 3249 | 5% | <0.001 |
| BRAF | 3 | 1% | 3249 | 5% | <0.001 |
| RAD21 | 13 | 2% | 1716 | 3% | 0.828 |
| RICTOR | 17 | 3% | 3127 | 5% | 0.087 |
| CCND1 | 17 | 3% | 3004 | 5% | 0.116 |
| DNMT3A | 16 | 3% | 3004 | 5% | 0.083 |
| NSD3 | 1 | 0% | 1584 | 3% | <0.001 |
| FGF19 | 16 | 3% | 2820 | 5% | 0.144 |
| ATM | 21 | 4% | 2820 | 5% | 0.697 |
| FGF3 | 15 | 3% | 2820 | 5% | 0.087 |
| SETD2 | 60 | 11% | 1778 | 3% | <0.001 |
| MDM2 | 53 | 10% | 2698 | 4% | <0.001 |
| CDK4 | 27 | 5% | 2085 | 3% | 0.063 |
| SMAD4 | 26 | 5% | 1839 | 3% | 0.026 |
| CTNNB1 | 23 | 4% | 1839 | 3% | 0.101 |
| FRS2 | 22 | 4% | 0 | 0% | <0.001 |
| ARFRP1 | 15 | 3% | 920 | 2% | 0.030 |
| FGF4 | 15 | 3% | 2698 | 4% | 0.138 |
| GNAS | 13 | 2% | 1410 | 2% | 0.826 |
| CHEK2 | 13 | 2% | 1042 | 2% | 0.210 |
| ALK ** | 0 | 0% | 1839 | 3% | <0.001 |
| ROS1 ** | 0 | 0% | 552 | 1% | 0.030 |
| ERBB2 ** | 0 | 0% | 2452 | 4% | 4.43E−09 |

*Includes any genes in the top 30 most frequently mutated in either group.
** Targetable driver fusions/amplifications in NSCLC.

In RET fusion-positive non-NSCLC tumors, TP53 (39%), CDKN2A (22%), CDKN2B (17%), TERT (14%), APC (8%), RNF43 (8%), PTEN (7%), MTAP (6%), SMAD4 (6%), and MLL2 (6%), were the 10 most frequently altered genes. See, Table 21. These genes varied among the various non-NSCLC tumor types. See, FIGS. 6B-6D. When comparing the NSCLC RET fusion-positive with the non-NSCLC RET fusion-positive cohort, significant differences were observed in several of the gene alteration frequencies. See, Table 21.

TABLE 21
Genomic alterations and significant associations among
RET fusion-positive NSCLC and non-NSCLC cohorts.

| | | | Other Solid | | |
|---|---|---|---|---|---|
| | NSCLC | | Tumors | | Corrected |
| Gene | (n = 523) | n | (n = 368) | n | P-Value |
| TP53 | 42.6% | 223 | 39.1% | 144 | 1 |
| CDKN2A | 29.3% | 153 | 22.0% | 81 | 0.497 |
| CDKN2B | 22.9% | 120 | 16.8% | 62 | 0.847 |
| MYC | 9.9% | 52 | 5.4% | 20 | 0.519 |
| SETD2 | 11.5% | 60 | 2.2% | 8 | <0.001 |
| MTAP | 7.6% | 40 | 6.3% | 23 | 1 |
| TERT | 1.9% | 10 | 13.6% | 50 | <0.001 |
| MDM2 | 10.1% | 53 | 0.8% | 3 | <0.001 |
| PTEN | 4.8% | 25 | 6.5% | 24 | 1 |
| SMAD4 | 5.0% | 26 | 6.0% | 22 | 1 |
| NKX2-1 | 7.1% | 37 | 1.1% | 4 | <0.001 |
| APC | 1.7% | 9 | 7.9% | 29 | <0.001 |
| ARID1A | 3.1% | 16 | 6.0% | 22 | 1 |
| ATM | 4.0% | 21 | 3.8% | 14 | 1 |
| CCND1 | 3.3% | 17 | 4.3% | 16 | 1 |
| RNF43 | 0.8% | 4 | 7.6% | 28 | <0.001 |
| FGF19 | 3.1% | 16 | 4.1% | 15 | 1 |
| NFKBIA | 5.4% | 28 | 0.8% | 3 | 0.004 |
| MLL2 | 1.5% | 8 | 6.0% | 22 | 0.010 |
| FGF3 | 2.9% | 15 | 4.1% | 15 | 1 |
| FGF4 | 2.9% | 15 | 4.1% | 15 | 1 |
| PIK3CA | 2.7% | 14 | 4.1% | 15 | 1 |
| CDK4 | 5.2% | 27 | 0.3% | 1 | <0.001 |
| RB1 | 3.6% | 19 | 2.2% | 8 | 1 |
| CTNNB1 | 4.4% | 23 | 1.1% | 4 | 0.139 |
| EGFR | 3.3% | 17 | 2.4% | 9 | 1 |
| FRS2 | 4.2% | 22 | 1.1% | 4 | 0.223 |
| DNMT3A | 3.1% | 16 | 1.9% | 7 | 1 |
| GNAS | 2.5% | 13 | 2.4% | 9 | 1 |
| RICTOR | 3.3% | 17 | 1.4% | 5 | 1 |

### Co-NCCN Guideline Driver Alterations Among RET Fusion-Positive NSCLC

Targetable co-alterations in RET fusion-positive NSCLC listed in the National Comprehensive Cancer Network (NCCN) Guidelines were examined. The specific NCCN genomic alterations examined were sensitizing EGFR mutations, KRAS G12C, BRAF V600E, ERBB2 mutations, MET exon 14 skipping mutations, ALK and ROS1 rearrangements, NTRK fusions, and MET amplifications. Overall, only 34 cases had co-occurring NCCN-NSCLC driver alterations. These included EGFR (3%, 17/223), KRAS (3%, 14/223), and BRAF (1%, 3/223).

### Immune Checkpoint Inhibitor Biomarkers

Immune checkpoint inhibitor (ICPI) biomarkers based on comprehensive genomic profiling (CGP) and PD-L1 IHC were examined. The NSCLC RET fusion-positive cohort had a significantly lower number of TMB-H cases and median TMB when compared to the NSCLC RET fusion-negative cohort (P<0.001). See, Table 17. In comparison to the NSCLC RET fusion-positive cohort, the non-NSCLC RET fusion-positive cohort had a significantly higher proportion of TMB-H cases, though the median TMB did not differ significantly (P<0.001 and P=0.741, respectively). See, Table 14.

No cases had a microsatellite instability-high (MSI-H) status in the overall NSCLC RET fusion-positive cohort. In comparison, 219 (0.4%) NSCLC RET fusion-negative cases were MSI-H and 29 (7.9%) non-NSCLC RET fusion-positive cases were MSI-H. See, Tables 14 and 17. Of note, 40% (25/65) of the RET fusion-positive colorectal carcinomas with a reportable MSI status were MSI-H.

Among 141 RET fusion-positive NSCLC cases tested for PD-L1 expression using the DAKO PD-L1 22C3 assay, 22% (31/141) had a negative TPS score (TPS<1%), 42% (59/141) had a low positive TPS score (TPS 1-49), and 36% (51/141) had a high positive score (TPS≥50). PD-L1 tumor cell expression in the NSCLC RET fusion-positive cohort was significantly higher than in the NSCLC RET fusion-negative cohort (P<0.001). See, Table 17. In addition, 95 RET fusion-positive non-NSCLC cases were tested for PD-L1 expression with the DAKO 22C3 assay and scored with TPS. In the non-NSCLC cohort, 60% (57/95) had a negative TPS score (TPS<1%), 25% (24/95) had a low positive TPS score (TPS 1-49), and 15% (14/95) had a high positive score (TPS≥50). See, Table 14.

### Prevalence of Tissue and Liquid RET Fusion Detection

Among 891 total RET fusion positive cases, twenty-three (23) cases were also tested with a liquid NGS assay. The median interval between specimen collection was 75 days, of which 10 out of 23 (43.5%) patients had a liquid assay performed after initial tissue-based NGS assay, 11 out of 23 (47.8%) had liquid assay as a primary comprehensive molecular NGS assay followed by tissue-based NGS, and 2 out of 23 (8.7%) had liquid and solid-based NGS at the same time point.

### CONCLUSIONS

The results described in this Example represent the largest single cohort of patients with RET fusion-positive solid tumors characterized by a DNA hybrid capture-based molecular assay. The results showed that RET fusion-positive NSCLC patients were enriched for Central and South American, East Asian, and South Asian patients when compared to the RET fusion-negative NSCLC cohort. The majority of RET fusions were mutually exclusive to other primary driver genomic alterations, and TMB was lower but PD-L1 expression trended higher in the NSCLC RET fusion-positive cohort when compared to the NSCLC RET fusion negative cohort, suggesting limited efficacy of ICPIs in RET fusion-positive NSCLC. In addition, no differences in the RET fusion breakpoints among NSCLC and other solid tumors were observed, and a high rate of MSI-H in patients with colorectal cancer (CRC) driven by RET fusions was observed. Further, the results described above are consistent with other studies that have suggested that RET fusions in lung cancer correlate with adenocarcinoma histology, younger age, never smoker status and advanced disease. See, Wang, R., et al., J Clin Oncol, 2012. 30 (35): p. 4352-9; Mukhopadhyay, S., et al., J Thorac Oncol, 2014. 9 (11): p. 1714-9; Tsuta, K., et al., Br J Cancer, 2014. 110 (6): p. 1571-8; Gautschi, O., et al., J Clin Oncol, 2017. 35 (13): p. 1403-1410; Pietrantonio, F., et al., JNCI: Journal of the National Cancer Institute, 2017. 109 (12); and Pietrantonio, F., et al., Annals of Oncology, 2018. 29 (6): p. 1394-1401.

### Additional RET Fusions

Additional RET gene fusions identified by CGP, and the corresponding cancer types in which the fusions were identified, are provided in Table 22.

TABLE 22
Detailed characteristics of additional RET gene fusions identified by CGP.

| | 5′ breakpoint/ | | Fusion transcript |
|---|---|---|---|
| Fusion | 3′ breakpoint | | sequence |
| Exon and breakpoint information | 5′ Exons | 5′ Refseq ID/ | Fusion protein |
| Tumor type | 3′ Exons | 3′ Refseq ID | sequence |
| ANKRD26 - RET | chr10: 27310264- | NM_014915/ | SEQ ID NO: 39 |
| 5′-ANKRD26(ex1-29 NM_014915)- | 27310361/ | NM_020630 | SEQ ID NO: 105 |
| RET(ex12-19 NM_020630); | chr10: 43610383- | | |
| Breakpoints: ANKRD26 intron 29, | 43610480 | | |
| RET intron 11 | ANKRD26 ex1-29 | | |
| Lung adenocarcinoma | RET ex12-19 | | |
| Reciprocal: No | | | |
| In frame fusion, includes RET kinase | | | |
| domain. | | | |
| Chr10 inversion fragment. | | | |
| CLIP1 - RET | chr12: 122805302- | NM_002956/ | SEQ ID NO: 40 |
| 5′-CLIP1(ex1-16 NM_002956)- | 122805302/ | NM_020630 | SEQ ID NO: 106 |
| RET(ex12-19 NM_020630); | chr10: 43611109- | | |
| Breakpoints: CLIP1 intron 16, RET | 43611109 | | |
| intron 11 | CLIP1 ex1-16 | | |
| Lung adenocarcinoma | RET ex12-19 | | |
| In frame fusion, includes RET kinase | | | |
| domain. | | | |
| DLG5 - RET | chr10: 79582000- | NM_004747/ | SEQ ID NO: 41 |
| 5′-DLG5(ex1-14 NM_004747)- | 79582000/ | NM_020630 | SEQ ID NO: 107 |
| RET(ex12-19 NM_020630); | chr10: 43611570- | | |
| Breakpoints: DLG5 intron 14, RET | 43611570 | | |
| intron 11 | DLG5 ex1-14 | | |
| Ovary serous carcinoma | RET ex12-19 | | |
| In frame fusion, includes RET kinase | | | |
| domain. | | | |
| Chr10 inversion fragment. | | | |
| DLG5 - RET | chr10: 79559942- | NM_004747/ | SEQ ID NO: 42 |
| 5′-DLG5(ex1-27 NM_004747)- | 79559942/ | NM_020630 | SEQ ID NO: 108 |
| RET(ex8-19 NM_020630); | chr10: 43607485- | | |
| Breakpoints: DLG5 intron 27, RET | 43607485 | | |
| intron 7 | DLG5 ex1-27 | | |
| Breast carcinoma (NOS) | RET ex8-19 | | |
| In frame fusion, includes RET kinase | | | |
| domain. | | | |
| Chr10 inversion fragment. | | | |
| ERC1 - RET | chr12: 1346557- | NM_178039/ | SEQ ID NO: 43 |
| 5′-ERC1(ex1-12 NM_178039)- | 1346557/ | NM_020630 | SEQ ID NO: 109 |
| RET(ex12-19 NM_020630); | chr10: 43610879- | | |
| Breakpoints: ERC1 intron 12, RET | 43610879 | | |
| intron 11 | ERC1 ex1-12 | | |
| Soft tissue sarcoma (NOS) | RET ex12-19 | | |
| In frame fusion, includes RET kinase | | | |
| domain. | | | |
| ERC1 - RET | chr12: 1290627- | NM_178039/ | SEQ ID NO: 44 |
| 5′-ERC1(ex1-8 NM_178039)- | 1290627/ | NM_020630 | SEQ ID NO: 110 |
| RET(ex12-19 NM_020630); | chr10: 43611066- | | |
| Breakpoints: ERC1 intron 8, RET | 43611066 | | |
| intron 11 | ERC1 ex1-8 | | |
| Unknown primary carcinoma (CUP) | RET ex12-19 | | |
| (NOS) | | | |
| Reciprocal: No | | | |
| In frame fusion, includes RET kinase | | | |
| domain. | | | |
| ERC1 - RET | chr12: 1350447- | NM_178039/ | SEQ ID NO: 45 |
| 5′-ERC1(ex1-12 NM_178039)- | 1350447/ | NM_020630 | SEQ ID NO: 111 |
| RET(ex11-19 NM_020630); | chr10: 43609823- | | |
| Breakpoints: ERC1 intron 12, RET | 43609823 | | |
| intron 10 | ERC1 ex1-12 | | |
| Pancreatobiliary carcinoma | RET ex11-19 | | |
| Includes RET kinase domain. | | | |
| FRMD4A - RET | chr10: 13758406- | NM_018027/ | SEQ ID NO: 46 |
| 5′-FRMD4A(ex1-12 NM_018027)- | 13758406/ | NM_020630 | SEQ ID NO: 112 |
| RET(ex12-19 NM_020630); | chr10: 43610354- | | |
| Breakpoints: FRMD4A intron 12, RET | 43610354 | | |
| intron 11 | FRMD4A ex1-12 | | |
| Unknown primary adenocarcinoma | RET ex12-19 | | |
| In frame fusion, includes RET kinase | | | |
| domain. | | | |
| Chr10 inversion fragment. | | | |
| KIAA1468 - RET | chr18: 59908435- | NM_020854/ | SEQ ID NO: 47 |
| 5′-KIAA1468(ex1-10 NM_020854)- | 59908435/chr10: | NM_020630 | SEQ ID NO: 113 |
| RET(ex12-19 NM_020630); | 43611083-43611083 | | |
| Breakpoints: KIAA1468 intron 10, RET | KIAA1468 ex1-10 | | |
| intron 11 | RET ex12-19 | | |
| Thyroid papillary carcinoma | | | |
| Reciprocal: Yes | | | |
| In frame fusion, includes RET kinase | | | |
| domain. | | | |
| NCOA4 - RET | chr10: 51585637- | NM_005437/ | SEQ ID NO: 48 |
| 5′-NCOA4(ex1-8 NM_005437)- | 51585735/ | NM_020630 | SEQ ID NO: 114 |
| RET(ex12-19 NM_020630) | chr10: 43610257- | | |
| Breakpoints NCOA4 intron 8, RET | 43610403 | | |
| intron 11 | NCOA4 ex1-8 | | |
| Colon adenocarcinoma (CRC) | RET ex12-19 | | |
| In frame fusion, includes RET kinase | | | |
| domain. | | | |
| Chr10 duplication fragment. | | | |
| PARD3 - RET | chr10: 34926445- | NM_019619/ | SEQ ID NO: 49 |
| 5′-PARD3(ex1-2 NM_019619)- | 34926445/ | NM_020630 | SEQ ID NO: 115 |
| RET(ex8-19 NM_020630); | chr10: 43607138- | | |
| Breakpoints: PARD3 intron 2, RET | 43607138 | | |
| intron 7 | PARD3 ex1-2 | | |
| Unknown primary urothelial carcinoma | RET ex8-19 | | |
| Reciprocal: Yes | | | |
| Includes RET kinase domain. | | | |
| Chr10 inversion fragment. | | | |
| PARD3 - RET | chr10: 34755707- | NM_019619/ | SEQ ID NO: 50 |
| 5′-PARD3(ex1-4 NM_019619)- | 34755707/ | NM_020630 | SEQ ID NO: 116 |
| RET(ex12-19 NM_020630); | chr10: 43612003- | | |
| Breakpoints: PARD3 intron 4, RET | 43612003 | | |
| intron 11 | PARD3 ex1-4 | | |
| Unknown primary neuroendocrine | RET ex12-19 | | |
| tumor | | | |
| Reciprocal: No | | | |
| In frame fusion, includes RET kinase | | | |
| domain. | | | |
| Chr10 inversion fragment. | | | |
| PRKAR1A - RET | chr17: 66523509- | NM_002734/ | SEQ ID NO: 51 |
| 5′-PRKAR1A(ex1-7 NM_002734)- | 66523607/ | NM_020630 | SEQ ID NO: 117 |
| RET(ex12-19 NM_020630); | chr10: 43611401- | | |
| Breakpoints: PRKAR1A intron 7, RET | 43611502 | | |
| intron 11 | PRKAR1A ex1-7 | | |
| Brain astrocytoma | RET ex12-19 | | |
| Reciprocal: Yes | | | |
| In frame fusion, includes RET kinase | | | |
| domain. | | | |
| PRKG1 - RET | chr10: 53889856- | NM_006258/ | SEQ ID NO: 52 |
| 5′-PRKG1(ex1-7 NM_006258)- | 53889954/ | NM_020630 | SEQ ID NO: 118 |
| RET(ex4-19 NM_020630) Breakpoints | chr10: 43600288- | | |
| PRKG1 intron 7, RET intron 3 | 43600402 | | |
| Prostate acinar adenocarcinoma | PRKG1 ex1-7 | | |
| Includes RET kinase domain. | RET ex4-19 | | |
| Chr10 duplication fragment. | | | |
| PRKG1 - RET | chr10: 53789188- | NM_006258/ | SEQ ID NO: 53 |
| 5′-PRKG1(ex1-5 NM_006258)- | 53789284/ | NM_020630 | SEQ ID NO: 119 |
| RET(ex18-19 NM_020630); | chr10: 43620162- | | |
| Breakpoints: PRKG1 intron 5, RET | 43620329 | | |
| intron 17 | PRKG1 ex1-5 | | |
| Lung adenocarcinoma | RET ex18-19 | | |
| Reciprocal: No | | | |
| Chr10 duplication fragment. | | | |
| RUFY2 - RET | chr10: 70143736- | NM_017987/ | SEQ ID NO: 54 |
| 5′-RUFY2(ex1-9 NM_017987)- | 70143833/ | NM_020630 | SEQ ID NO: 120 |
| RET(ex12-19 NM_020630); | chr10: 43611405- | | |
| Breakpoints: RUFY2 intron 9, RET | 43611519 | | |
| intron 11 | RUFY2 ex1-9 | | |
| Lung adenocarcinoma | RET ex12-19 | | |
| In frame fusion, includes RET kinase | | | |
| domain. | | | |
| Chr10 inversion fragment. | | | |
| SNRNP70 - RET | chr19: 49602640- | NM_003089/ | SEQ ID NO: 55 |
| 5′-SNRNP70(ex1-6 NM_003089)- | 49602640/ | NM_020630 | SEQ ID NO: 121 |
| RET(ex12-19 NM_020630); | chr10: 43611323- | | |
| Breakpoints: SNRNP70 intron 6, RET | 43611323 | | |
| intron 11 | SNRNP70 ex1-6 | | |
| Lung adenocarcinoma | RET ex12-19 | | |
| In frame fusion, includes RET kinase | | | |
| domain. | | | |
| SQSTM1 - RET | chr5: 179254189- | NM_003900/ | SEQ ID NO: 56 |
| 5′-SQSTM1(ex1-5 NM_003900)- | 179254286/ | NM_020630 | SEQ ID NO: 122 |
| RET(ex11-19 NM_020630); | chr10: 43609433- | | |
| Breakpoints: SQSTM1 intron 5, RET | 43609563 | | |
| intron 10 | SQSTM1 ex1-5 | | |
| Lung adenocarcinoma | RET ex11-19 | | |
| In frame fusion, includes RET kinase | | | |
| domain. | | | |
| TNIP1 - RET | chr5: 150416127- | NM_006058/ | SEQ ID NO: 57 |
| 5′-TNIP1(ex1-13 NM_006058)- | 150416127/ | NM_020630 | SEQ ID NO: 123 |
| RET(ex12-19 NM_020630); | chr10: 43611669- | | |
| Breakpoints: TNIP1 intron 13, RET | 43611669 | | |
| intron 11 | TNIP1 ex1-13 | | |
| Lung adenocarcinoma | RET ex12-19 | | |
| In frame fusion, includes RET kinase | | | |
| domain. | | | |
| TNIP1 - RET | chr5: 150414253- | NM_006058/ | SEQ ID NO: 58 |
| 5′-TNIP1(ex1-15 NM_006058)- | 150414351/ | NM_020630 | SEQ ID NO: 124 |
| RET(ex12-19 NM_020630); | chr10: 43611007- | | |
| Breakpoints: TNIP1 intron 15, RET | 43611109 | | |
| intron 11 | TNIP1 ex1-15 | | |
| Lung adenocarcinoma | RET ex12-19 | | |
| Reciprocal: No | | | |
| In frame fusion, includes RET kinase | | | |
| domain. | | | |
| TNIP1 - RET | chr5: 150417592- | NM_006058/ | SEQ ID NO: 59 |
| 5′-TNIP1(ex1-12 NM_006058)- | 150417592/ | NM_020630 | SEQ ID NO: 125 |
| RET(ex12-19 NM_020630); | chr10: 43611428- | | |
| Breakpoints: TNIP1 intron 12, RET | 43611428 | | |
| intron 11 | TNIP1 ex1-12 | | |
| Lung adenocarcinoma | RET ex12-19 | | |
| Reciprocal: No | | | |
| In frame fusion, includes RET kinase | | | |
| domain. | | | |
| TRIM27 - RET | chr6: 28881702- | NM_006510/ | SEQ ID NO: 60 |
| 5′-TRIM27(ex1-3 NM_006510)- | 28881890/ | NM_020630 | SEQ ID NO: 126 |
| RET(ex12-19 NM_020630); | chr10: 43610709- | | |
| Breakpoints: TRIM27 intron 3, RET | 43610878 | | |
| intron 11 | TRIM27 ex1-3 | | |
| Colon adenocarcinoma (CRC) | RET ex12-19 | | |
| In frame fusion, includes RET kinase | | | |
| domain. | | | |
| TRIM33 - RET | chr1: 114965871- | NM_015906/ | SEQ ID NO: 61 |
| 5′-TRIM33(ex1-10 NM_015906)- | 114965970/ | NM_020630 | SEQ ID NO: 127 |
| RET(ex12-19 NM_020630); | chr10: 43611514- | | |
| Breakpoints: TRIM33 intron 10, RET | 43611626 | | |
| intron 11 | TRIM33 ex1-10 | | |
| Pancreas ductal adenocarcinoma | RET ex12-19 | | |
| Reciprocal: No | | | |
| In frame fusion, includes RET kinase | | | |
| domain. | | | |
| CCDC6 - RET | chr10: 61585545- | NM_005436/ | SEQ ID NO: 62 |
| 5′-CCDC6(ex1-3 NM_005436)- | 61585642/ | NM_020630 | SEQ ID NO: 128 |
| RET(ex11-19 NM_020630): | chr10: 43609686- | | |
| Breakpoints: CCDC6 intron 3, RET | 43609783 | | |
| intron 10 | CCDC6 ex1-3 | | |
| Ovary epithelial carcinoma | RET ex11-19 | | |
| Reciprocal: No | | | |
| Includes RET kinase domain. | | | |
| Chr10 inversion fragment. | | | |
| TRIM24 - RET | chr7: 138267911- | NM_003852/ | SEQ ID NO: 63 |
| 5′-TRIM24(ex1-17 NM_003852)- | 138267911/ | NM_020630 | SEQ ID NO: 129 |
| RET(ex12-19 NM_020630); | chr10: 43611642- | | |
| Breakpoints: TRIM24 intron 17, RET | 43611642 | | |
| intron 11 | TRIM24 ex1-17 | | |
| Lung adenocarcinoma | RET ex12-19 | | |
| Reciprocal: Yes | | | |
| In frame fusion, includes RET kinase | | | |
| domain. | | | |
| FGFR1OP - RET | chr6: 167424630- | NM_007045/ | SEQ ID NO: 64 |
| 5′-FGFR1OP(ex1-6 NM_007045)- | 167424630/ | NM_020630 | SEQ ID NO: 130 |
| RET(ex12-19 NM_020630); | chr10: 43611296- | | |
| Breakpoints: FGFR1OP intron 6, RET | 43611296 | | |
| intron 11 | FGFR1OP ex1-6 | | |
| Unknown primary adenocarcinoma | RET ex12-19 | | |
| In frame fusion, includes RET kinase | | | |
| domain. | | | |
| GAS2 - RET | chr11: 22812170- | NM_005256/ | SEQ ID NO: 65 |
| 5′-GAS2(ex1-6 NM_005256)-RET(ex4- | 22812170/ | NM_020630 | SEQ ID NO: 131 |
| 19 NM_020630); Breakpoints: GAS2 | chr10: 43600385- | | |
| intron 6, RET intron 3 | 43600385 | | |
| Lung non-small cell lung carcinoma | GAS2 ex1-6 | | |
| (NSCLC) (NOS) | RET ex4-19 | | |
| Includes RET kinase domain. | | | |
| HOOK1 - RET | chr1: 60334563- | NM_015888/ | SEQ ID NO: 66 |
| 5′-HOOK1(ex1-20 NM_015888)- | 60334563/ | NM_020630 | SEQ ID NO: 132 |
| RET(ex12-19 NM_020630); | chr10: 43610286- | | |
| Breakpoints: HOOK1 intron 20, RET | 43610286 | | |
| intron 11 | HOOK1 ex1-20 | | |
| Intra-hepatic cholangiocarcinoma | RET ex12-19 | | |
| In frame fusion, includes RET kinase | | | |
| domain. | | | |
| LMNA - RET | chr1: 156102332- | NM_005572/ | SEQ ID NO: 147 |
| 5′-LMNA(ex1-2 NM_005572)- | 156102332/ | NM_020630 | SEQ ID NO: 137 |
| RET(ex12-19 NM_020630); | chr10: 43611407- | | |
| Breakpoints: LMNA intron 2, RET | 43611407 | | |
| intron 11 | LMNA ex1-2 | | |
| Thyroid papillary carcinoma | RET ex12-19 | | |
| In frame fusion, includes RET kinase | | | |
| domain. | | | |
| LMNA - RET | chr1: 156102293- | NM_005572/ | SEQ ID NO: 148 |
| 5′-LMNA(ex1-2 NM_005572)- | 156102386/ | NM_020630 | SEQ ID NO: 138 |
| RET(ex12-19 NM_020630); | chr10: 43611365- | | |
| Breakpoints: LMNA intron 2, RET | 43611462 | | |
| intron 11 | LMNA ex1-2 | | |
| Thyroid papillary carcinoma | RET ex12-19 | | |
| In frame fusion, includes RET kinase | | | |
| domain. | | | |
| MLPH - RET | chr2: 238445254- | NM_024101/ | SEQ ID NO: 149 |
| 5′-MLPH(ex1-9 NM_024101)- | 238445254/ | NM_020630 | SEQ ID NO: 139 |
| RET(ex12-19 NM_020630); | chr10: 43610453- | | |
| Breakpoints: MLPH intron 9, RET | 43610453 | | |
| intron 11 | MLPH ex1-9/ | | |
| Unknown primary melanoma | RET ex12-19 | | |
| In frame fusion, includes RET kinase | | | |
| domain. | | | |
| MYH9 - RET | chr22: 36683797- | NM_002473/ | SEQ ID NO: 150 |
| 5′-MYH9(ex1-34 NM_002473)- | 36683797/ | NM_020630 | SEQ ID NO: 140 |
| RET(ex12-19 NM_020630); | chr10: 43611848- | | |
| Breakpoints: MYH9 intron 34, RET | 43611848 | | |
| intron 11 | MYH9 ex1-34/ | | |
| Lung non-small cell lung carcinoma | RET ex12-19 | | |
| (NSCLC) (NOS) | | | |
| Reciprocal: Yes. | | | |
| In frame fusion, includes RET kinase | | | |
| domain. | | | |
| PCDH15 - RET | chr10: 56509282- | NM_033056/ | SEQ ID NO: 151 |
| 5′-PCDH15(ex1-1 UTR* NM_033056)- | 56509282/ | NM_020630 | SEQ ID NO: 141 |
| RET(ex5-19 NM_020630); | chr10: 43601819- | | |
| Breakpoints: PCDH15 intron 1, RET | 43601819 | | |
| intron 4 | PCDH15 ex1-1/ | | |
| *UTR indicates that exon 1 in PCDH15 | RET ex5-19 | | |
| is typically untranslated in its canonical | | | |
| transcript. | | | |
| Lung adenocarcinoma | | | |
| Includes RET kinase domain. | | | |
| Chr10 inversion fragment. | | | |
| PIBF1 - RET | chr13: 73550847- | NM_006346/ | SEQ ID NO: 152 |
| 5′-PIBF1(ex1-16 NM_006346)- | 73550847/ | NM_020630 | SEQ ID NO: 142 |
| RET(ex12-19 NM_020630) | chr10: 43611532- | | |
| Breakpoints PIBF1 intron 16, RET | 43611532 | | |
| intron 11 | PIBF1 ex1-16/ | | |
| Lung adenocarcinoma | RET ex12-19 | | |
| Reciprocal: Yes. | | | |
| In frame fusion, includes RET kinase | | | |
| domain. | | | |
| PIBF1 - RET | chr13: 73548007- | NM_006346/ | SEQ ID NO: 153 |
| 5′-PIBF1(ex1-16 NM_006346)- | 73548007/ | NM_020630 | SEQ ID NO: 143 |
| RET(ex12-19 NM_020630) | chr10: 43611167- | | |
| Breakpoints PIBF1 intron 16, RET | 43611167 | | |
| intron 11 | PIBF1 ex1-16 | | |
| Lung adenocarcinoma | RET ex12-19 | | |
| Reciprocal: Yes. | | | |
| In frame fusion, includes RET kinase | | | |
| domain. | | | |
| SLC12A2 - RET | chr5: 127496774- | NM_001046/ | SEQ ID NO: 154 |
| 5′-SLC12A2(ex1-16 NM_001046)- | 127496774/ | NM_020630 | SEQ ID NO: 144 |
| RET(ex12-19 NM_020630); | chr10: 43611855- | | |
| Breakpoints: SLC12A2 intron 16, RET | 43611855 | | |
| intron 11 | SLC12A2 ex1-16 | | |
| Lung adenocarcinoma | RET ex12-19 | | |
| Reciprocal: Yes. | | | |
| In frame fusion, includes RET kinase | | | |
| domain. | | | |
| ZNF485 - RET | chr10: 44109385- | NM_145312/ | SEQ ID NO: 155 |
| 5′-ZNF485(ex1-4 NM_145312)- | 44109456/ | NM_020630 | SEQ ID NO: 145 |
| RET(ex12-19 NM_020630) | chr10: 43611964- | | |
| Breakpoints ZNF485 intron 4, RET | 43612093 | | |
| intron 11 | ZNF485 ex1-4 | | |
| Cervix squamous cell carcinoma (SCC) | RET ex12-19 | | |
| Includes RET kinase domain. | | | |
| Chr10 duplication fragment. | | | |
| ZNF485 - RET | chr10: 44109396- | NM_145312/ | SEQ ID NO: 156 |
| 5′-ZNF485(ex1-4 NM_145312)- | 44109396/ | NM_020630 | SEQ ID NO: 146 |
| RET(ex10-19 NM_020630) | chr10: 43608521- | | |
| Breakpoints ZNF485 intron 4, RET | 43608521 | | |
| intron 9 | ZNF485 ex1-4 | | |
| Lung adenocarcinoma | RET ex10-19 | | |
| In frame fusion, includes RET kinase | | | |
| domain. | | | |
| Chr10 duplication fragment. | | | |

### Example 3: Clinicopathologic and Biomarker Characteristics of RET Fusion-Positive versus RET Fusion-Negative Papillary Thyroid Carcinomas and Colon Adenocarcinomas

This Example describes the differences in age between RET fusion-positive versus RET fusion-negative patients with either papillary thyroid carcinoma (PTC) or colon adenocarcinoma. The age of the RET fusion positive cohort was significantly younger than the RET fusion negative PTC cohort (33 vs. 62 years old, p<0.001). See, Table 23. There was a trend in the opposite direction for colon adenocarcinoma, although a smaller absolute value difference (66 vs 60 years old, p<0.001). See, Table 24.

TABLE 23
Comparison of clinicopathologic and genomic biomarker
characteristics of RET fusion-positive and RET
fusion-negative Papillary Thyroid Carcinoma

| | RET fus+ | | RET fus− | | |
|---|---|---|---|---|---|
| | Papillary | | Papillary | | |
| | Thyroid | | Thyroid | | |
| | Carcinoma | | Carcinoma | | |
| | (n = 109) | | (n = 1090) | | p value |
| Age, median | 33 | [22-56] | 62 | [52-71] | <0.001 |
| years [IQR]* | | | | | |
| Sex, Female/Male | 68/41 | (62%/38%) | 560/530 | (51%/49%) | 0.028 |
| Specimen Site | | | | | 0.433 |
| Primary | 49 | (45%) | 422 | (39%) | |
| Metastatic | 52 | (48%) | 571 | (52%) | |
| Unknown | 8 | (7%) | 97 | (9%) | |
| Genetic | | | | | 0.159 |
| Ancestry** | | | | | |
| African | 10 | (9%) | 55 | (5%) | |
| Central and South | 24 | (22%) | 202 | (19%) | |
| American | | | | | |
| East Asian | 5 | (5%) | 79 | (7%) | |
| European | 67 | (62%) | 739 | (68%) | |
| South Asian | 3 | (3%) | 15 | (1%) | |
| ICPI biomarkers | | | | | |
| PD-L1 (DAKO | n = 27 | | n = 261 | | 0.992 |
| 22C3)*** | | | | | |
| <1 | 14 | (52%) | 135 | (52%) | |
| 1-49 | 9 | (33%) | 85 | (33%) | |
| ≥50 | 4 | (15%) | 41 | (16%) | |
| TMB-H | 0 | (0.0%) | 17 | (1.6%) | 0.189 |
| TMB, median | 0 | [0.0-1.3] | 1.3 | [0.0-2.5] | 0.7413 |
| muts/Mb | | | | | |
| MSI-H | 0 | (0%) | 0 | (0%) | 1 |

Abbreviations: Rearranged during transfection (RET). Non-small-cell lung cancer (NSCLC). Programmed death-ligand 1/Cluster of Differentiation 274 (PD-L1). Tumor Proportion Score (TPS). Tumor mutational burden-High (TMB-H). Microsatellite instability-high (MSI-H).
Other Solid Tumors exclude NSCLC.
*Wilcox Rank Sum Test
**p-value adjusted for multiple comparisons;
***Chi-squared Test

TABLE 24
Comparison of clinicopathologic and genomic biomarker characteristics
of RET fusion-positive and RET fusion negative colon adenocarcinoma

| | RET fus+ Colon | | RET fus− Colon | | |
|---|---|---|---|---|---|
| | adenocarcinoma | | adenocarcinoma | | |
| | (n = 60) | | (n = 32,938) | | p value |
| Age, median years [IQR]* | 66 | [63-75] | 60 | [51-69] | <0.001 |
| Sex, Female/Male/unknown | 30/30/0 | (50%/50%/0) | 15,343/17,573/22 | | 0.854 |
| | | | (47%/53%/0.1%) | | |
| Specimen Site | | | | | 0.021 |
| Primary | 40 | (66.7%) | 16090 | (48.8%) | |
| Metastatic | 20 | (33.3%) | 16753 | (50.9%) | |
| Unknown | 0 | (0.0%) | 95 | (0.3%) | |
| Genetic ancestry** | | | | | 0.812 |
| African | 5 | (8.3%) | 4067 | (12.3%) | |
| Central and South American | 8 | (13.3%) | 3182 | (9.7%) | |
| East Asian | 3 | (5.0%) | 1376 | (4.2%) | |
| European | 43 | (71.7%) | 24033 | (73.0%) | |
| South Asian | 1 | (1.7%) | 271 | (0.8%) | |
| ICPI biomarkers | | | | | |
| PD-L1 (Dako 22C3)*** | n = 19 | | n = 7234 | | 0.006 |
| <1 | 12 | (63%) | 6216 | (86%) | |
| 1-49 | 7 | (37%) | 907 | (13%) | |
| ≥50 | 0 | (0%) | 111 | (2%) | |
| TMB-H | 31 | (51.7%) | 3075 | (9.3%) | <0.001 |
| TMB, median muts/Mb | 10 | [5.2-40] | 3.8 | [2.5-6.1] | <0.001 |
| MSI-H | 25 | (41.7%) | 1805 | (5.5%) | <0.001 |

Abbreviations: Rearranged during transfection (RET). Non-small-cell lung cancer (NSCLC). Programmed death-ligand 1/Cluster of Differentiation 274 (PD-L1). Tumor Proportion Score (TPS). Tumor mutational burden-High (TMB-H). Microsatellite instability-high (MSI-H).
Other Solid Tumors exclude NSCLC.
*Wilcox Rank Sum Test
**p-value adjusted for multiple comparisons;
***Chi-squared Test

### Example 4: Genomic Alterations and Significant Associations Among RET Fusion-Positive and Fusion-Negative Papillary Thyroid Carcinoma (PTC) and Colon Adenocarcinoma

This Example describes gene mutations found in RET fusion-positive and RET fusion-negative papillary thyroid carcinoma or colon adenocarcinoma. When comparing the RET fusion-positive PTC cohort, a lower frequency of BRAF, TERT, NRAS, and PIK3CA genomic alterations were observed, compared to the RET fusion-negative PTC cohort (P<0.05). See, Table 25. Lastly, when the RET fusion-positive colon adenocarcinoma cohort was examined, a higher frequency of RNF43, MLL2, CASP8, CREBBP, BCORL1, SPEN, SMARCA4, BRCA2, MSH3, PTCH1, QKI, EP300, LRP1B, CDH1, and FANCA; but a lower frequency of APC, KRAS, PIK3CA, and BRAF genomic alterations were observed, compared to the RET fusion-negative colon adenocarcinoma cohort. See. Table 26. (P<0.05).

TABLE 25
Comparative Genomics: Genomic alterations and significant
associations among RET fusion-positive and fusion-
negative papillary thyroid carcinoma (PTC)

| | RET fusion | | RET fusion | | |
|---|---|---|---|---|---|
| | positive PTC | | negative PTC | | corrected |
| Gene* | (n = 109) | n | (n = 1090) | n | p-value |
| BRAF | 0% | 0 | 78% | 855 | <0.001 |
| TERT | 21% | 23 | 64% | 702 | <0.001 |
| CDKN2A | 8% | 9 | 10% | 107 | 0.829 |
| TP53 | 5% | 5 | 10% | 106 | 0.417 |
| NRAS | 0% | 0 | 7% | 81 | 0.006 |
| PIK3CA | 0% | 0 | 7% | 75 | 0.011 |
| CDKN2B | 6% | 6 | 6% | 61 | 1 |
| RBM10 | 3% | 3 | 5% | 50 | 0.816 |
| DNMT3A | 1% | 1 | 4% | 49 | 0.417 |
| ATM | 1% | 1 | 3% | 37 | 0.619 |
| STAG2 | 0% | 0 | 2% | 26 | 0.564 |
| CHEK2 | 3% | 3 | 2% | 23 | 0.829 |
| MTAP | 3% | 3 | 2% | 22 | 0.816 |
| NTRK1 | 0% | 0 | 2% | 20 | 0.619 |
| KRAS | 0% | 0 | 2% | 19 | 0.749 |
| ARID1A | 0% | 0 | 2% | 18 | 0.749 |
| BCOR | 0% | 0 | 2% | 18 | 0.749 |
| TET2 | 1% | 1 | 2% | 17 | 1 |
| MEN1 | 0% | 0 | 2% | 17 | 0.749 |
| PTEN | 2% | 2 | 2% | 17 | 0.829 |
| MUTYH | 2% | 2 | 1% | 16 | 0.829 |
| HRAS | 1% | 1 | 1% | 15 | 1 |
| NUP93 | 0% | 0 | 1% | 14 | 0.816 |
| AKT1 | 0% | 0 | 1% | 13 | 0.816 |
| U2AF1 | 0% | 0 | 1% | 12 | 0.816 |
| AKT2 | 0% | 0 | 1% | 12 | 0.816 |
| ASXL1 | 0% | 0 | 1% | 11 | 0.816 |
| BCORL1 | 1% | 1 | 1% | 10 | 1 |
| NF2 | 3% | 3 | 1% | 10 | 0.467 |
| APC | 0% | 0 | 1% | 10 | 0.816 |
| TBX3 | 2% | 2 | 0% | 2 | 0.305 |
| BRCA1 | 2% | 2 | 1% | 10 | 0.698 |
| EP300 | 2% | 2 | 1% | 7 | 0.617 |
| SETD2 | 2% | 2 | 1% | 6 | 0.564 |
| BRCA2 | 2% | 2 | 1% | 8 | 0.619 |

TABLE 26
Comparative Genomics: Genomic alterations and significant associations
among RET fusion-positive and fusion-negative colon adenocarcinoma

| | RET fusion | | RET fusion | | |
|---|---|---|---|---|---|
| | positive colon | | negative colon | | |
| | adenocarcinoma | | adenocarcinoma | | corrected |
| Gene* | (n = 60) | n | (n = 32,938) | n | p-value |
| APC | 37% | 22 | 77% | 25288 | <0.001 |
| TP53 | 65% | 39 | 75% | 24644 | 0.188 |
| KRAS | 2% | 1 | 50% | 16523 | <0.001 |
| PIK3CA | 3% | 2 | 19% | 6368 | 0.002 |
| SMAD4 | 20% | 12 | 16% | 5248 | 0.467 |
| SOX9 | 15% | 9 | 11% | 3647 | 0.387 |
| BRAF | 0% | 0 | 10% | 3416 | 0.007 |
| FBXW7 | 13% | 8 | 9% | 3106 | 0.354 |
| PTEN | 10% | 6 | 9% | 2804 | 0.670 |
| MYC | 7% | 4 | 8% | 2501 | 1.000 |
| FLT3 | 2% | 1 | 7% | 2385 | 0.216 |
| FAM123B | 8% | 5 | 7% | 2377 | 0.663 |
| ARID1A | 17% | 10 | 7% | 2359 | 0.023 |
| CDK8 | 2% | 1 | 7% | 2345 | 0.216 |
| GNAS | 3% | 2 | 7% | 2176 | 0.513 |
| RNF43 | 40% | 24 | 6% | 2036 | <0.001 |
| ATM | 7% | 4 | 5% | 1731 | 0.609 |
| BCL2L1 | 0% | 0 | 5% | 1599 | 0.216 |
| ERBB2 | 3% | 2 | 5% | 1556 | 1.000 |
| ASXL1 | 8% | 5 | 4% | 1468 | 0.303 |
| CTNNB1 | 2% | 1 | 4% | 1468 | 0.587 |
| NRAS | 0% | 0 | 4% | 1345 | 0.291 |
| PIK3R1 | 5% | 3 | 4% | 1316 | 0.587 |
| MLL2 | 28% | 17 | 4% | 1314 | <0.001 |
| IRS2 | 0% | 0 | 3% | 1113 | 0.354 |
| SMAD2 | 0% | 0 | 3% | 1066 | 0.354 |
| ZNF217 | 0% | 0 | 3% | 1039 | 0.354 |
| AURKA | 0% | 0 | 3% | 1030 | 0.354 |
| ARFRP1 | 0% | 0 | 3% | 991 | 0.354 |
| SRC | 0% | 0 | 3% | 946 | 0.508 |
| CASP8 | 17% | 10 | 2% | 511 | <0.001 |
| CREBBP | 13% | 8 | 2% | 810 | 0.001 |
| BCORL1 | 12% | 7 | 3% | 881 | 0.004 |
| SPEN | 12% | 7 | 1% | 359 | <0.001 |
| SMARCA4 | 12% | 7 | 2% | 517 | <0.001 |
| BRCA2 | 10% | 6 | 3% | 882 | 0.014 |
| MSH3 | 10% | 6 | 2% | 638 | 0.004 |
| PTCH1 | 10% | 6 | 2% | 516 | 0.002 |
| QKI | 10% | 6 | 1% | 389 | <0.001 |
| EP300 | 8% | 5 | 2% | 589 | 0.012 |
| LRP1B | 8% | 5 | 2% | 563 | 0.011 |
| CDKN2A | 7% | 4 | 3% | 897 | 0.159 |
| NF1 | 7% | 4 | 2% | 817 | 0.127 |
| CDH1 | 7% | 4 | 1% | 406 | 0.016 |
| FLCN | 7% | 4 | 2% | 625 | 0.062 |
| MSH6 | 7% | 4 | 2% | 693 | 0.081 |
| FANCA | 7% | 4 | 1% | 267 | 0.005 |

What is claimed is:

1. A method of treating or delaying progression of cancer, comprising:
(a) detecting a RET fusion nucleic acid molecule, or a RET fusion polypeptide encoded by the RET fusion nucleic acid molecule, in a sample from an individual having a cancer, wherein:
(i) the RET fusion nucleic acid molecule comprises a fusion between a RET gene, or a portion thereof, and a gene listed in Table 1, or a portion thereof, or
(ii) the RET fusion nucleic acid molecule comprises a fusion between a RET gene, or a portion thereof, and a gene listed in Table 2, or a portion thereof, and the cancer is the cancer corresponding to the gene listed in Table 2; and
(b) administering to the individual an effective amount of a treatment that comprises a RET-targeted therapy.

2. A method of identifying one or more treatment options for an individual having a cancer, the method comprising:
(a) detecting in a sample from the individual a RET fusion nucleic acid molecule, or a RET fusion polypeptide encoded by the RET fusion nucleic acid molecule, wherein:
(i) the RET fusion nucleic acid molecule comprises a fusion between a RET gene, or a portion thereof, and a gene listed in Table 1, or a portion thereof, or
(ii) the RET fusion nucleic acid molecule comprises a fusion between a RET gene, or a portion thereof, and a gene listed in Table 2, or a portion thereof, and the cancer is the cancer corresponding to the gene listed in Table 2; and
(b) generating a report comprising one or more treatment options identified for the individual based at least in part on detection of the RET fusion nucleic acid molecule, or the RET fusion polypeptide encoded by the RET fusion nucleic acid molecule, in the sample, wherein the one or more treatment options comprise a RET-targeted therapy.

3. A method of detecting a RET fusion nucleic acid molecule, the method comprising:
(a) providing a sample from an individual having a cancer, wherein the sample comprises a plurality of nucleic acid molecules;
(b) preparing a nucleic acid sequencing library from the plurality of nucleic acid molecules in the sample;
(c) amplifying said library;
(d) selectively enriching for one or more nucleic acid molecules comprising nucleotide sequences corresponding to a RET fusion nucleic acid molecule in said library to produce an enriched sample, wherein:
(i) the RET fusion nucleic acid molecule comprises a fusion between a RET gene, or a portion thereof, and a gene listed in Table 1, or a portion thereof, or
(ii) the RET fusion nucleic acid molecule comprises a fusion between a RET gene, or a portion thereof, and a gene listed in Table 2, or a portion thereof, and the cancer is the cancer corresponding to the gene listed in Table 2;
(e) sequencing the enriched sample, thereby producing a plurality of sequence reads;
(f) analyzing the plurality of sequence reads for the presence of the RET fusion nucleic acid molecule; and
(g) detecting, based on the analyzing step, the presence or absence of the RET fusion nucleic acid molecule in the sample from the individual.

4. The method of claim 1, wherein:
(a) the RET fusion nucleic acid molecule is a RET fusion nucleic acid molecule listed in Table 3, and wherein the order of the genes in the fusion, in 5′ to 3′ direction, is as listed in Table 3;
(b) the RET fusion nucleic acid molecule is a RET fusion nucleic acid molecule listed in Table 4, and wherein the RET fusion nucleic acid molecule comprises or results from a corresponding 5′ breakpoint and/or 3′ breakpoint within the exons or introns as listed in Table 4;
(c) the RET fusion nucleic acid molecule is a RET fusion nucleic acid molecule listed in Table 5, and wherein the RET fusion nucleic acid molecule comprises or results from a corresponding 5′ breakpoint within the chromosomal coordinates as listed in Table 5, and/or a corresponding 3′ breakpoint within the chromosomal coordinates as listed in Table 5;
(d) the RET fusion nucleic acid molecule is a RET fusion nucleic acid molecule listed in Table 6, and wherein the RET fusion nucleic acid molecule comprises or results from a fusion of a corresponding 5′ exon as listed in Table 6, or a portion thereof, fused to a corresponding 3′ exon as listed in Table 6, or a portion thereof;
(e) the RET fusion nucleic acid molecule is a RET fusion nucleic acid molecule listed in Table 7, and wherein the RET fusion nucleic acid molecule comprises, in 5′ to 3′ direction, the corresponding exons or portions thereof as listed in Table 7;
(f) the RET fusion nucleic acid molecule is a RET fusion nucleic acid molecule listed in Table 8, and wherein the RET fusion nucleic acid molecule comprises a corresponding nucleotide sequence as listed in Table 8, or a nucleotide sequence with at least about 70% homology thereto; and/or
(g) the RET fusion nucleic acid molecule comprises a nucleotide sequence encoding a corresponding RET fusion polypeptide as listed in Table 9, wherein the RET fusion polypeptide comprises a corresponding amino acid sequence as listed in Table 9, or an amino acid sequence with at least about 70% homology thereto.

5. The method of claim 1, wherein:
(a) the RET fusion polypeptide encoded by the RET fusion nucleic acid molecule comprises a RET kinase domain, or a fragment of a RET kinase domain having RET kinase activity;
(b) the RET fusion polypeptide encoded by the RET fusion nucleic acid molecule has RET kinase activity;
(c) the RET fusion polypeptide encoded by the RET fusion nucleic acid molecule has a constitutive RET kinase activity;
(d) the RET fusion polypeptide encoded by the RET fusion nucleic acid molecule is oncogenic;
(e) the RET fusion polypeptide encoded by the RET fusion nucleic acid molecule promotes cancer cell survival, angiogenesis, cancer cell proliferation, and any combination thereof; and/or
(f) the RET fusion polypeptide encoded by the RET fusion nucleic acid molecule is capable of dimerizing with a RET polypeptide or with another RET fusion polypeptide.

6. The method of claim 1, wherein:
(a) the RET fusion nucleic acid molecule is a RET fusion nucleic acid molecule comprising a fusion between a RET gene, or a portion thereof, and a gene listed in Table 1, or a portion thereof, and wherein the cancer is a solid tumor;
(b) the RET fusion nucleic acid molecule is a RET fusion nucleic acid molecule comprising a fusion between a RET gene, or a portion thereof, and a gene listed in Table 1, or a portion thereof, and wherein the cancer is a hematologic malignancy;
(c) the RET fusion nucleic acid molecule is a RET fusion nucleic acid molecule comprising a fusion between a RET gene, or a portion thereof, and a gene listed in Table 1, or a portion thereof, and wherein the cancer is a lymphoma;
(d) the RET fusion nucleic acid molecule is a RET fusion nucleic acid molecule comprising a fusion between a RET gene, or a portion thereof, and a gene listed in Table 1, or a portion thereof, and wherein the cancer is an ovarian cancer, a thyroid cancer, an adenocarcinoma, a breast cancer, a lung cancer, a colon cancer, a carcinoma, a uterine cancer, a prostate cancer, a pancreatic cancer, a leiomyosarcoma, a sarcoma, an esophageal cancer, a brain cancer, a bladder cancer, a skin cancer, a cervical cancer, or a melanoma;
(e) the RET fusion nucleic acid molecule is a RET fusion nucleic acid molecule comprising a fusion between a RET gene, or a portion thereof, and a gene listed in Table 1, or a portion thereof, and the cancer is ovary epithelial carcinoma, thyroid papillary carcinoma, unknown primary adenocarcinoma, breast carcinoma, lung non-small cell lung carcinoma, colon adenocarcinoma, unknown primary carcinoma, breast invasive ductal carcinoma, uterus endometrial adenocarcinoma mixed histology, prostate acinar adenocarcinoma, lung squamous cell carcinoma, lung small cell undifferentiated carcinoma, pancreas ductal adenocarcinoma, bladder urothelial (transitional cell) carcinoma, soft tissue leiomyosarcoma, soft tissue sarcoma, esophagus adenocarcinoma, ovary serous carcinoma, colon neuroendocrine carcinoma, brain glioblastoma, breast carcinoma, unknown primary malignant neoplasm, lung adenocarcinoma, unknown primary cancer, unknown primary serous carcinoma, thyroid carcinoma, uterus carcinosarcoma, pancreatobiliary carcinoma, unknown primary urothelial carcinoma, neuroendocrine tumor, unknown primary neuroendocrine tumor, brain astrocytoma, cholangiocarcinoma, intra-hepatic cholangiocarcinoma, cervix squamous cell carcinoma, or unknown primary melanoma;
(f) the RET fusion nucleic acid molecule is a RET fusion nucleic acid molecule comprising a fusion between a RET gene, or a portion thereof, and a gene listed in Table 1, or a portion thereof, and wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pincaloma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor; or
(g) the RET fusion nucleic acid molecule is a RET fusion nucleic acid molecule comprising a fusion between a RET gene, or a portion thereof, and a gene listed in Table 1, or a portion thereof, and wherein the cancer is acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

7. The method of claim 1, wherein the RET fusion nucleic acid molecule is a RET fusion nucleic acid molecule comprising a fusion between a RET gene, or a portion thereof, and a gene listed in Table 1, or a portion thereof, and wherein:
(a) the RET fusion nucleic acid molecule is a RET fusion nucleic acid molecule as listed in Table 3, and wherein the order of the genes in the fusion, in 5′ to 3′ direction, is as listed in Table 3;
(b) the RET fusion nucleic acid molecule is a RET fusion nucleic acid molecule as listed in Table 4, and wherein the RET fusion nucleic acid molecule comprises or results from a corresponding 5′ breakpoint and/or 3′ breakpoint within the exons or introns as listed in Table 4;
(c) the RET fusion nucleic acid molecule is a RET fusion nucleic acid molecule as listed in Table 5, and wherein the RET fusion nucleic acid molecule comprises or results from a corresponding 5′ breakpoint within the chromosomal coordinates as listed in Table 5, and/or a corresponding 3′ breakpoint within the chromosomal coordinates as listed in Table 5;
(d) the RET fusion nucleic acid molecule is a RET fusion nucleic acid molecule as listed in Table 6, and wherein the RET fusion nucleic acid molecule comprises or results from a fusion of a corresponding 5′ exon as listed in Table 6, or a portion thereof, fused to a corresponding 3′ exon as listed in Table 6, or a portion thereof;
(e) the RET fusion nucleic acid molecule is a RET fusion nucleic acid molecule as listed in Table 7, and wherein the RET fusion nucleic acid molecule comprises, in 5′ to 3′ direction, the corresponding exons or portions thereof as listed in Table 7;
(f) the RET fusion nucleic acid molecule is a RET fusion nucleic acid molecule as listed in Table 8, and wherein the RET fusion nucleic acid molecule comprises a corresponding nucleotide sequence as listed in Table 8, or a nucleotide sequence with at least about 70% homology thereto; and/or
(g) the RET fusion nucleic acid molecule comprises a nucleotide sequence encoding a corresponding RET fusion polypeptide as listed in Table 9, wherein the RET fusion polypeptide comprises a corresponding amino acid sequence as listed in Table 9, or an amino acid sequence with at least about 70% homology thereto; and
wherein the cancer is the corresponding cancer as listed in Table 10.

8. The method of claim 1, wherein the RET-targeted therapy comprises one or more of a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for RET-positive or RET-rearranged cancer, a RET-targeted therapy being tested in a clinical trial, a treatment for RET-positive or RET-rearranged cancer being tested in a clinical trial, or any combination thereof.

9. The method of claim 1, wherein the RET-targeted therapy is a kinase inhibitor, optionally a tyrosine kinase inhibitor, a multi-kinase inhibitor, or a RET-specific inhibitor.

10. The method of claim 9, wherein the RET-targeted therapy comprises one or more of pralsetinib, selpercatinib, lenvatinib, sorafenib, sunitinib, vandetanib, NVP-AST487, regorafenib, dovitinib, motesanib, cabozantinib, lapatinib, lestaurtinib, linifanib, semaxinib, ponatinib, fostamatinib, quizartinib, imatinib, vatalanib, ENMD-2076, JNJ-26483327, DCC-2157, Zeteletinib, TPX0046, TAS0953, RXDX-105, LOXO-260, BOS172738, Alectinib, APS03118, LOX-18228, or SYHA1815.

11. The method of claim 8, wherein:
(a) the nucleic acid inhibits the expression of the RET fusion nucleic acid molecule or the RET fusion polypeptide encoded by the RET fusion nucleic acid molecule, wherein optionally the nucleic acid is a double-stranded RNA (dsRNA), a small interfering RNA (siRNA), or a small hairpin RNA (shRNA); or
(b) the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

12. The method of claim 1, wherein the individual has received a prior anti-cancer treatment, or is being treated with an anti-cancer treatment; wherein optionally the RET fusion nucleic acid molecule, and/or the RET fusion polypeptide encoded by the RET fusion nucleic acid molecule, confers resistance of the cancer to the anti-cancer treatment.

13. The method of claim 1, wherein:
(a) the cancer has not been previously treated, wherein optionally the RET-targeted therapy comprises one or more of pralsetinib, selpercatinib, lenvatinib, sorafenib, sunitinib, vandetanib, NVP-AST487, regorafenib, dovitinib, motesanib, cabozantinib, lapatinib, lestaurtinib, linifanib, semaxinib, ponatinib, fostamatinib, quizartinib, imatinib, vatalanib, ENMD-2076, JNJ-26483327, DCC-2157, Zeteletinib, TPX0046, TAS0953, RXDX-105, LOXO-260, BOS172738, Alectinib, APS03118, LOX-18228, or SYHA1815;
(b) wherein the RET-targeted therapy is a first-line or front-line treatment, wherein optionally the RET-targeted therapy comprises one or more of pralsetinib, selpercatinib, lenvatinib, sorafenib, sunitinib, vandetanib, NVP-AST487, regorafenib, dovitinib, motesanib, cabozantinib, lapatinib, lestaurtinib, linifanib, semaxinib, ponatinib, fostamatinib, quizartinib, imatinib, vatalanib, ENMD-2076, JNJ-26483327, DCC-2157, Zeteletinib, TPX0046, TAS0953, RXDX-105, LOXO-260, BOS172738, Alectinib, APS03118, LOX-18228, or SYHA1815;
(c) the cancer is kinase inhibitor-naïve; and/or
(d) the cancer has not been previously treated with a kinase inhibitor.

14. The method of claim 1, wherein:
(a) the cancer has been previously treated with a kinase inhibitor;
(b) the cancer progressed on a prior treatment with a kinase inhibitor;
(c) the cancer is refractory to a prior kinase inhibitor treatment; and/or
(d) the cancer progressed on a prior treatment with a chemotherapy and a kinase inhibitor;
wherein optionally the kinase inhibitor is a tyrosine kinase inhibitor, multi-kinase inhibitor, or a RET-specific inhibitor.

15. The method of claim 1, wherein:
(a) the sample is obtained from the cancer;
(b) the sample comprises a tissue biopsy sample, a liquid biopsy sample, or a normal control, wherein optionally the sample is from a tumor biopsy, tumor specimen, or circulating tumor cell;
(c) the sample is a liquid biopsy sample and comprises blood, plasma, cerebrospinal fluid, sputum, stool, urine, or saliva, wherein optionally the sample comprises circulating tumor cells (CTCs) or cell-free DNA (cfDNA) and/or circulating tumor DNA (ctDNA);
(d) the sample comprises cells and/or nucleic acids from the cancer, wherein optionally the sample comprises mRNA, DNA, circulating tumor DNA (ctDNA), cell-free DNA, or cell-free RNA from the cancer.

16. The method of claim 1, wherein the RET fusion nucleic acid molecule is detected in the sample by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing.

17. The method of claim 16, wherein:
(a) the sequencing comprises a massively parallel sequencing (MPS) technique, whole genome sequencing (WGS), whole exome sequencing, targeted sequencing, direct sequencing, or a Sanger sequencing technique; and optionally wherein the massively parallel sequencing (MPS) technique comprises next-generation sequencing (NGS); and/or
(b) the method further comprises selectively enriching for one or more nucleic acid molecules in the sample comprising nucleotide sequences corresponding to the RET fusion nucleic acid molecule.

18. The method of claim 17, wherein the selectively enriching produces an enriched sample, and optionally the selectively enriching comprises:
(a) combining one or more bait molecules with the sample, thereby hybridizing the one or more bait molecules to one or more nucleic acids in the sample comprising nucleotide sequences corresponding to the RET fusion nucleic acid molecule and producing nucleic acid hybrids; and isolating the nucleic acid hybrids to produce the enriched sample; or
(b) amplifying the one or more nucleic acid molecules comprising nucleotide sequences corresponding to the RET fusion nucleic acid molecule using a polymerase chain reaction (PCR) to produce an enriched sample.

19. The method of claim 18, wherein the one or more bait molecules comprise a capture nucleic acid molecule configured to hybridize to a nucleotide sequence corresponding to the RET fusion nucleic acid molecule; and wherein optionally the capture nucleic acid molecule comprises a DNA, RNA, or mixed DNA/RNA molecule.

20. The method of claim 1, wherein the individual is a human.


SEQUENCE LISTING (156 sequences)

SEQ ID NO: 1 - skipped

SEQ ID NO: 2 - DNA, 2472 bp, synthetic construct
  source 1..2472: mol_type = other DNA
atgctgagccgattatcaggattagcaaatgttgttttgcatgaattatcaggagatgatgacactgatcagaatatgagggctcccctagaccctgaattacaccaagaatctgacatggaatttaataatactacacaagaagatgttcaggagcgcctggcttatgcagagcaattggtggtggagctaaaagatattattagacagaaggatgttcaactgcagcagaaagatgaagctctacaggaagagagaaaagctgctgataacaaaattaaaaaactaaaacttcatgcgaaggccaaattaacttctttgaataaatacatagaagaaatgaaagcacaaggagggactgttctgcctacagaacctcagtcagaggagcaactttccaagcatgacaagagttctacagaggaagagatggaaatagaaaagataaaacataagctccaggagaaggaggaactaatcagcactttgcaagcccagcttactcaggcacaggcagaacaacctgcacagagttctacagagatggaagaatttgtaatgatgaagcaacagctccaggagaaggaagaattcattagcactttacaagcccagctcagccagacacaggcagagcaagctgcacagcaggtggtccgagagaaagatgcccgctttgaaacacaagttcgtcttcatgaagatgagcttcttcagttagtaacccaggcagatgtggaaacagagatgcaacagaaattgagggtgctgcaaaggaagcttgaggaacacgaagaatccttggtgggccgtgctcaggtcgttgacttgctgcaacaggagctgactgctgctgagcagagaaaccagattctctctcagcagttacagcagatggaagctgagcataatactttgaggaacactgtggaaacagaaagagaggagtccaagattctactggaaaagatggaacttgaagtggcagagagaaaattatccttccataatctgcaggaagaaatgcatcatcttttagaacagtttgagcaagcaggccaagcccaggctgaactagagtctcggtatagtgctttggagcagaagcacaaagcagaaatggaagagaagacctctcatattttgagtcttcaaaagactggacaagagctgcagtctgcctgtgatgctctaaaggatcaaaattcaaagcttctccaagataagaatgagcaagcagttcagtcagcccagaccattcagcaactggaagatcagctccagcaaaaatccaaagaaattagccaatttctaaatagactgcccttgcaacaacatgaaacagcatctcagacttctttcccagatgtttataatgagggcacacaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 3 - DNA, 1986 bp, synthetic construct
  source 1..1986: mol_type = other DNA
atgcctcaccttatggaaaggatggtgggctctggcctcctgtggctggccttggtctcctgcattctgacccaggcatctgcagtgcagcgaggttatggaaaccccattgaagccagttcgtatgggctggacctggactgcggagctcctggcaccccagaggctcatgtctgttttgacccctgtcagaattacaccctcctggatgaacccttccgaagcacagagaactcagcagggtcccaggggtgcgataaaaacatgagcggctggtaccgctttgtaggggaaggaggagtaaggatgtcggagacctgtgtccaggtgcaccgatgccagacagacgctcccatgtggctgaatgggacccaccctgcccttggggatggcatcaccaaccacactgcctgtgcccattggagtggcaactgctgtttctggaaaacagaggtgctggtgaaggcctgcccaggcgggtaccatgtgtaccggttggaaggcactccctggtgtaatctgagatactgcacagacccatccactgtggaggacaagtgtgagaaggcctgccgccccgaggaggagtgccttgccctcaacagcacctggggctgtttctgcagacaggacctcaatagttctgatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 4 - DNA, 339 bp, synthetic construct
  source 1..339: mol_type = other DNA
atgtcgtcgtcgtcgccgccggcgggggctgccagcgccgccatctcggcctcggagaaagtggacggcttcacccggaaatcggtccgcaaggcgcagaggcagaagcgctcccagggctcgtcgcagtttcgcagccagggcagccaggcagagctgcacccgctgccccagctcaaaggcatgtcagacccgaactggcctggagagagtcctgtaccactcacgagagctgatggcactaacactgggtttccaagatatccaaatgatagtgtatatgctaactggatgctttcaccctcagcggcaaaattaatggacacgtttgatagttaa

SEQ ID NO: 5 - skipped

SEQ ID NO: 6 - DNA, 5580 bp, synthetic construct
  source 1..5580: mol_type = other DNA
atggtccagctgaggaagctgctccgcgtcctgactttgatgaagttcccctgctgcgtgctggaggtgctcctgtgcgcgctggcggcggcggcgcgcggccaggagatgtacgccccgcactcaatccggatcgagggggacgtcaccctcggggggctgttccccgtgcacgccaagggtcccagcggagtgccctgcggcgacatcaagagggaaaacgggatccacaggctggaagcgatgctctacgccctggaccagatcaacagtgatcccaacctactgcccaacgtgacgctgggcgcgcggatcctggacacttgttccagggacacttacgcgctcgaacagtcgcttactttcgtccaggcgctcatccagaaggacacctccgacgtgcgctgcaccaacggcgaaccgccggttttcgtcaagccggagaaagtagttggagtgattggggcttcggggagttcggtctccatcatggtagccaacatcctgaggctcttccagatcccccagattagttatgcatcaacggcacccgagctaagtgatgaccggcgctatgacttcttctctcgcgtggtgccacccgattccttccaagcccaggccatggtagacattgtaaaggccctaggctggaattatgtgtctaccctcgcatcggaaggaagttatggagagaaaggtgtggagtccttcacgcagatttccaaagaggcaggtggactctgcattgcccagtccgtgagaatcccccaggaacgcaaagacaggaccattgactttgatagaattatcaaacagctcctggacacccccaactccagggccgtcgtgatttttgccaacgatgaggatataaagcagatccttgcagcagccaaaagagctgaccaagttggccattttctttgggtgggatcagacagctggggatccaaaataaacccactgcaccagcatgaagatatcgcagaaggggccatcaccattcagcccaagcgagccacggtggaagggtttgatgcctactttacgtcccgtacacttgaaaacaacagaagaaatgtatggtttgccgaatactgggaggaaaacttcaactgcaagttgacgattagtgggtcaaaaaaagaagacacagatcgcaaatgcacaggacaggagagaattggaaaagattccaactatgagcaggagggtaaagtccagttcgtgattgacgcagtctatgctatggctcacgcccttcaccacatgaacaaggatctctgtgctgactaccggggtgtctgcccagagatggagcaagctggaggcaagaagttgctgaagtatatacgcaatgttaatttcaatggtagtgctggcactccagtgatgtttaacaagaacggggatgcacctgggcgttatgacatctttcagtaccagaccacaaacaccagcaacccgggttaccgtctgatcgggcagtggacagacgaacttcagctcaatatagaagacatgcagtggggtaaaggagtccgagagatacccgcctcagtgtgcacactaccatgtaagccaggacagagaaagaagacacagaaaggaactccttgctgttggacctgtgagccttgcgatggttaccagtaccagtttgatgagatgacatgccagcattgcccctatgaccagaggcccaatgaaaatcgaaccggatgccaggatattcccatcatcaaactggagtggcactccccctgggctgtgattcctgtcttcctggcaatgttggggatcattgccaccatctttgtcatggccactttcatccgctacaatgacacgcccattgtccgggcatctgggcgggaactcagctatgttcttttgacgggcatctttctttgctacatcatcactttcctgatgattgccaaaccagatgtggcagtgtgttctttccggcgagttttcttgggcttgggtatgtgcatcagttatgcagccctcttgacgaaaacaaatcggatttatcgcatatttgagcagggcaagaaatcagtaacagctcccagactcataagcccaacatcacaactggcaatcacttccagtttaatatcagttcagcttctaggggtgttcatttggtttggtgttgatccacccaacatcatcatagactatgatgaacacaagacaatgaaccctgagcaagccagaggggttctcaagtgtgacattacagatctccaaatcatttgctccttgggatatagcattcttctcatggtcacatgtactgtgtatgccatcaagactcggggtgtacccgagaattttaacgaagccaagcccattggattcactatgtacacgacatgtatagtatggcttgccttcattccaattttttttggcaccgctcaatcagcggaaaagctctacatacaaactaccacgcttacaatctccatgaacctaagtgcatcagtggcgctggggatgctatacatgccgaaagtgtacatcatcattttccaccctgaactcaatgtccagaaacggaagcgaagcttcaaggcggtagtcacagcagccaccatgtcatcgaggctgtcacacaaacccagtgacagacccaacggtgaggcaaagaccgagctctgtgaaaacgtagacccaaacaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 7 - skipped

SEQ ID NO: 8 - DNA, 6429 bp, synthetic construct
  source 1..6429: mol_type = other DNA
atggcagccgtgaccatgtcggtgcccgggcggaaggcgccccccaggccgggcccagtgcccgaggcggcccagccgttcctgttcacgccccgcgggcccagcgcgggtggcgggcctggctcgggcacctccccgcaggtggagtggacggcccggcgtctcgtgtgggtgccttcggagcttcacgggttcgaggcggcggcgctgcgggacgaaggcgaggaggaggcggaggtggagctggcggagagcgggaggcggctgcgactgccgcgggaccagatccagcgcatgaacccgcccaagttcagcaaggccgaggacatggccgagctgacctgcctcaacgaggcctcggtcctgcacaacctccgggagcggtactactccggcctcatctacacgtactccggccttttctgtgtggtcatcaacccgtacaagcagcttcccatctacacagaagccattgtggagatgtaccggggcaagaagcgccacgaggtgccaccccacgtgtacgcagtgaccgagggggcctatcggagcatgctgcaggatcgtgaggaccagtccattctctgcactggagagtctggagctgggaagacggaaaacaccaagaaggtcatccagtacctcgcccacgtggcgtcgtctccaaagggcaggaaggagccgggtgtccccggtgagctggagcggcagctgcttcaggccaaccccatcctagaggcctttggcaatgccaagacagtgaagaatgacaactcctcccgattcggcaaattcatccgcatcaactttgatgttgccgggtacatcgtgggcgccaacattgagacctacctgctggagaagtcgcgggccatccgccaggccaaggacgagtgcagcttccacatcttctaccagctgctggggggcgctggagagcagctcaaagccgacctcctcctcgagccctgctcccactaccggttcctgaccaacgggccgtcatcctctcccggccaggagcgggaactcttccaggagacgctggagtcgctgcgggtcctgggattcagccacgaggaaatcatctccatgctgcggatggtctcagcagttctccagtttggcaacattgccttgaagagagaacggaacaccgatcaagccaccatgcctgacaacacagctgcacagaagctctgccgcctcttgggactgggggtgacggatttctcccgagccttgctcacccctcgcatcaaagttggccgagactatgtgcagaaagcccagactaaggaacaggctgacttcgcgctggaggccctggccaaggccacctacgagcgcctcttccgctggctggttctgcgcctcaaccgggccttggaccgcagcccccgccaaggcgcctccttcctgggcatcctggacatcgcgggctttgagatcttccagctgaactccttcgagcagctctgcatcaactacaccaacgagaagctgcagcagctcttcaaccacaccatgttcgtgctggagcaggaggagtaccagcgtgagggcatcccctggaccttcctcgactttggcctcgacctgcagccctgcatcgacctcatcgagcggccggccaacccccctggactcctggccctgctggatgaggagtgctggttcccgaaggccacagacaagtcgtttgtggagaaggtagcccaggagcagggcggccaccccaagttccagcggccgaggcacctgcgggatcaggccgacttcagtgttctccactacgcgggcaaggtcgactacaaggccaacgagtggctgatgaaaaacatggaccctctgaatgacaacgtcgcagccttgctccaccagagcacagaccggctgacggcagagatctggaaagacgtggagggcatcgtggggctggaacaggtgagcagcctgggcgacggcccaccaggtggccgcccccgtcggggtatgttccggacagtgggacagctctacaaggagtccctgagccgcctcatggccacactcagcaacaccaaccccagttttgtccgctgcattgtccccaaccacgagaagagggccgggaagctggagccacggctggtgctggaccagcttcgctgcaacggggtcctggagggcatccgcatctgtcgccagggcttccccaaccgcatcctcttccaggagttccggcagcgatacgagatcctgacacccaatgccatccccaagggcttcatggatgggaagcaggcctgtgaaaagatgatccaggcgctggaactggaccccaacctctaccgcgtgggacagagcaagatcttcttccgggctggggtcctggcccagctggaagaggagcgagacctgaaggtcaccgacatcatcgtctccttccaggcagctgcccggggatacctggctcgcagggccttccagaagcgccagcagcagcagagcgccctgagggtgatgcagcggaactgcgcggcctacctcaagctgagacactggcagtggtggcggctgtttaccaaggtgaagccactgctgcaggtgacgcggcaggatgaggtgctgcaggcacgggcccaggagctgcagaaagtgcaggagctacagcagcagagcgcccgcgaagttggggagctccagggccgagtggcacagctggaagaggagcgcgcccgcctggcagagcaattgcgagcagaggcagaactgtgtgcagaggccgaggagacgcgggggaggctggcagcccgcaagcaggagctggagctggtggtgtcagagctggaggctcgcgtgggcgaggaggaggagtgcagccgtcaaatgcaaaccgagaagaagaggctgcagcagcacatacaggagctagaggcccaccttgaggctgaggagggtgcgcggcagaagctgcagctggagaaggtgacgacagaggcaaaaatgaagaaatttgaagaggacctgctgctcctggaagaccagaattccaagctgagcaaggagcggaagctgctggaagatcgtctggccgagttctcatcccaggcagctgaggaggaggagaaggtcaagagcctcaataagctacggctcaaatatgaggccacaatcgcagacatggaggaccgcctacggaaggaggagaagggtcgccaggagctggagaagctgaagcggaggctggatggggagagctcagagctgcaggagcagatggtggagcagcaacagcgggcagaggagctgcgggcccagctgggccggaaggaggaggagctgcaggctgccctggccagggcagaagacgagggtggggcccgggcccagctgctgaaatccctgcgggaggctcaagcagccctggccgaggcccaggaggacctggagtctgagcgtgtggccaggaccaaggcggagaagcagcgccgggacctgggcgaggagctggaggcgctgcggggcgagctggaggacacgctggactccaccaacgcacagcaggagctccggtccaagagggaacaggaggtgacggagctgaagaagactctggaggaggagactcgcatccacgaggcggcagtgcaggagctgaggcagcgccacggccaggccctgggggagctggcggagcagctggagcaggcccggaggggcaaaggtgcatgggagaagacccggctggccctggaggccgaggtgtccgagctgcgggcagaactgagcagcctgcagactgcacgtcaggagggtgagcagcggaggcgccgcctggagttacagctgcaggaggtgcagggccgggctggtgatggggagagggcacgagcggaggctgctgagaagctgcagcgagcccaggctgaactggagaatgtgtctggggcgctgaacgaggctgagtccaaaaccatccgtcttagcaaggagctgagcagcacagaagcccagctgcacgatgcccaggagctgctgcaggaggagaccagggcgaaattggccttggggtcccgggtgcgagccatggaggctgaggcagccgggctgcgtgagcagctggaggaggaggcagctgccagggaacgggcgggccgtgaactgcagactgcccaggcccagctttccgagtggcggcggcgccaggaggaggaggcaggggcactggaggcaggggaggaggcacggcgccgggcagcccgggaggccgaggccctgacccagcgcctggcagaaaagacagagaccgtggatcggctggagcggggccgccgccggctgcagcaggagctggacgacgccaccatggacctggagcagcagcggcagcttgtgagcaccctggagaagaagcagcgcaagtttgaccagcttctggcagaggagaaggcagctgtacttcgggcagtggaggaacgtgagcgggccgaggcagagggccgggagcgtgaggctcgggccctgtcactgacacgggcactggaggaggagcaggaggcacgtgaggagctggagcggcagaaccgggccctgcgggctgagctggaggcactgctgagcagcaaggatgacgtcggcaagagcgtgcatgagctggaacgagcctgccgggtagcagaacaggcagccaatgatctgcgagcacaggtgacagaactggaggatgagctgacagcggccgaggatgccaagctgcgtctggaggtgactgtgcaggctctcaagactcagcatgagcgtgacctgcagggccgtgatgaggctggtgaagagaggcggaggcagctggccaagcagctgagagatgcagaggtggagcgggatgaggagcggaagcagcgcactctggccgtggctgcccgcaagaagctggagggagagctggaggagctgaaggctcagatggcctctgccggccagggcaaggaggaggcggtgaagcagcttcgcaagatgcaggcccagatgaaggagctatggcgggaggtggaggagacacgcacctcccgggaggagatcttctcccagaatcgggaaagtgaaaagcgcctcaagggcctggaggctgaggtgctgcggctgcaggaggaactggccgcctcggaccgtgctcggcggcaggcccagcaggaccgggatgagatggcagatgaggtggccaatggtaaccttagcaaggcagccattctggaggagaagcgtcagctggaggggcgcctggggcagttggaggaagagctggaggaggagcagagcaactcggagctgctcaatgaccgctaccgcaagctgctcctgcaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 9 - DNA, 3258 bp, synthetic construct
  source 1..3258: mol_type = other DNA
atggtgcagaaggagagtcaagcgacgttggaggagcgggagagcgagctcagctccaaccctgccgcctctgcgggggcatcgctggagccgccggcagctccggcacccggagaagacaaccccgccggggctgggggagcggcggtggccggggctgcaggaggggctcggcggttcctctgcggtgtggtggaaggattttatggaagaccttgggttatggaacagagaaaagaactctttagaaggctccagaaatgggaattaaatacatacttgtatgccccaaaagatgactacaaacataggatgttttggcgagagatgtattcagtggaggaagctgagcaacttatgactctcatctctgctgcacgagaatatgagatagagttcatctatgcgatctcacctggattggatatcactttttctaaccccaaggaagtatccacattgaaacgtaaattggaccaggtttctcagtttgggtgcagatcatttgctttgctttttgatgatatagaccataatatgtgtgcagcagacaaagaggtattcagttcttttgctcatgcccaagtctccatcacaaatgaaatctatcagtacctaggagagccagaaactttcctcttctgtcccacagaatactgtggcactttctgttatccaaatgtgtctcagtctccatatttaaggactgtgggtgaaaagcttctacctggaattgaagtgctttggacaggtcccaaagttgtttctaaagaaattccagtagagtccatcgaagaggtttctaagattattaagagagctccagtaatctgggataacattcatgctaatgattatgatcagaagagactgtttctgggcccgtacaaaggaagatccacagaactcatcccacggttaaaaggagtcctcactaatccaaattgtgaatttgaagccaactacgttgctatccacacccttgccacctggtacaaatcaaacatgaatggagtgagaaaagatgtagtgatgactgacagtgaagatagtactgtgtccatccagataaaattagaaaatgaaggcagtgatgaagatattgaaactgatgtactctatagtccacagatggctctaaagctagcattaacagaatggttgcaagagtttggtgtgcctcatcaatacagcagtaggcaagttgcacacagtggagctaaagcaagtgtagttgatgggactcctttagttgcagcaccctctttaaatgccacaaccgtagtaacaacagtttatcaggagcccattatgagccagggagcagccttgagtggtgagcctactactctgaccaaggaagaagaaaagaaacagcctgatgaagaacccatggacatggtggtggaaaaacaagaagaaacggaccacaagaatgacaatcaaatactgagtgaaattgttgaagcgaaaatggcagaggaattgaaaccaatggacactgataaagagagcatagctgaatcaaaatccccagagatgtccatgcaagaagattgtattagtgacattgcccccatgcaaactgatgaacagacaaacaaggagcagtttgtgccaggtccaaatgaaaagcctttgtacactgcggaaccagtgaccctggaggatttgcagttacttgctgatctattctaccttccttacgagcatggacccaaaggagcacagatgttacgggaatttcaatggcttcgagcaaatagtagtgttgtcagtgtcaattgcaaaggaaaagactctgaaaaaattgaagaatggcggtcacgagcagccaagtttgaagagatgtgtggactagtgatgggaatgttcactcggctctccaattgtgccaacaggacaattctttatgacatgtactcctatgtttgggatatcaagagtataatgtctatggtgaagtcttttgtacagtggttagggtgtcgtagtcattcttcagcacaattcttaattggagaccaagaaccctgggcctttagaggtggtctagcaggagagttccagcgtttgctgccaattgatggggcaaatgatctcttttttcagccacctccactgactcctacctccaaagtttatactatcagaccttattttcctaaggatgaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 10 - DNA, 1536 bp, synthetic construct
  source 1..1536: mol_type = other DNA
atgatgtttcgcgaccaggtcggggtgctggcgggctggtttaagggctggaacgagtgcgagcagactgttgcgctgctgtcgctgctcaagcgcgtgagccagacccaggcccgcttcctccagctctgcctggagcactcgctggccgactgcgccgagctgcacgtcctcgaacgcgaggccaacagccccgatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 11 - DNA, 2478 bp, synthetic construct
  source 1..2478: mol_type = other DNA
atgagagctgcacccctcctcctggccagggcagcaagccttagccttggcttcttgtttctgctttttttctggctagaccgaagtgtactagccaaggagttgaagtttgtgactttggtgtttcggcatggagaccgaagtcccattgacacctttcccactgaccccataaaggaatcctcatggccacaaggatttggccaactcacccagctgggcatggagcagcattatgaacttggagagtatataagaaagagatatagaaaattcttgaatgagtcctataaacatgaacaggtttatattcgaagcacagacgttgaccggactttgatgagtgctatgacaaacctggcagccctgtttcccccagaaggtgtcagcatctggaatcctatcctactctggcagcccatcccggtgcacacagttcctctttctgaagatcagttgctatacctgcctttcaggaactgccctcgttttcaagaacttgagagtgagactttgaaatcagaggaattccagaagaggctgcacccttataaggattttatagctaccttgggaaaactttcaggattacatggccaggacctttttggaatttggagtaaagtctacgaccctttatattgtgagagtgttcacaatttcactttaccctcctgggccactgaggacaccatgactaagttgagagaattgtcagaattgtccctcctgtccctctatggaattcacaagcagaaagagaaatctaggctccaagggggtgtcctggtcaatgaaatcctcaatcacatgaagagagcaactcagataccaagctacaaaaaactcatcatgtattctgcgcatgacactactgtgagtggcctacagatggcgctagatgtttacaacggactccttcctccctatgcttcttgccacttgacggaattgtactttgagaagggggagtactttgtggagatgtactatcggaatgagacgcagcacgagccgtatcccctcatgctacctggctgcagccccagctgtcctctggagaggtttgctgagctggttggccctgtgatccctcaagactggtccacggagtgtatgaccacaaacagccatcaagatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 12 - DNA, 3789 bp, synthetic construct
  source 1..3789: mol_type = other DNA
atggaggctaaggaccagaagaaacacagaaagaaaaacagtggacccaaagctgcaaagaaaaagaagcggcttctgcaggatctccagctaggagacgaagaagatgcccggaagagaaatcccaaagcttttgcagttcagtctgctgtgcggatggctcgatcctttcacaggactcaggatttgaagacaaaaaagcatcatattccagtggttgatcgaactccactagagcccccaccaatagtggtagtggtgatgggacctccaaaagttggaaagagcactttgatacaatgcctcattcggaactttacccggcagaagttgactgagatcagaggccctgtgacgattgtgtcaggtaaaaagcgcagactcaccattattgaatgtgggtgtgacattaacatgatgattgatctggctaaagtagcagatctggtactgatgcttatagatgccagctttgggtttgaaatggaaacgtttgagtttctaaacatctgtcaagtacatggctttcctaaaattatgggagttctcacccacctcgactccttcaagcataataagcaactgaagaagacaaagaagcgattaaaacacaggttctggacggaagtttacccgggtgccaagctgttctacctttctggaatggtgcatggagaatatcaaaaccaagaaatccacaatctgggccgttttattacagttatgaagtttaggcctctcacatggcaaacttctcacccttatatcctggcagacaggatggaagatttgacaaacccagaggatatccgaacaaacatcaaatgtgaccggaaggtgtcactttatggttatttaagaggagcacacttgaaaaataaaagccaaattcacatgccaggggtaggagattttgccgtgagtgacatcagtttcctcccagacccttgcgctcttcctgaacaacaaaagaagcgctgtttaaatgagaaggagaagctggtttatgcgcctctttctggagttgggggtgtgctgtatgacaaagacgctgtctatgttgaccttggtggcagccacgtttttcaggatgaagtggggcccacccatgagctggtccagagtctcatctctacccactccaccattgatgccaagatggcttcaagtcgagtgacgctgttttctgattccaagccacttgggtcagaggatatagataatcaagggctaatgatgccaaaggaggaaaaacaaatggacttgaacactggtcgaatgcgtcggaaagccattttcggagatgaagatgaatctggagatagtgatgatgaagaagatgatgaaatgtctgaagatgacgggttggaaaacggctctagtgatgaggaagcagaagaggaggaaaatgctgagatgactgatcagtatatggctgttaagggcatcaaacgacggaaacttgagttggaagaagacagtgaaatggatttgccagcatttgctgacagtgacgatgaccttgagaggagctcagcggaagaaggggaagcggaggaagctgatgaaagcagtgaagaagaggactgcactgcaggagagaagggcatttcaggatcaaaggctgctggagaaggtagtaaagcagggctgtcaccagctaattgccagagtgaccgtgtgaatctggagaagtctttgctgatgaagaaagcagctctccccactttcgattctgggcattgcacagctgaagaggtgtttgcatctgaagatgaatctgaagaaagctcctcactcagtgcagaggaagaagactcagaaaatgaagaggctattagaaaaaagctttcaaagccttctcaagtgagcagtggtcagaaactggggccacagaacttcattgatgagaccagtgatatagaaaatttactcaaagaggaagaagattacaaggaagaaaataatgattccaaagaaacgtcaggtgccctcaagtggaaggaagacctttccagaaaggcagctgaggcctttctgaggcagcagcaagcagctccaaacctccgaaagcttatttatgggacagtgacagaagataatgaagaagaagatgatgatactctagaagagcttggagggttgtttcgtgtcaaccagcctgacagagagtgtaagcacaaggctgactctttggactgctccagatttcttgtggaggccccccatgactgggatttagaggaggttatgaacagtatcagagattgcttcgtgactggaaagtgggaagatgataaagatgcagccaaggtcttagcagaagatgggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 13 - DNA, 2556 bp, synthetic construct
  source 1..2556: mol_type = other DNA
atgactacagctgtagagagaaagtatattaatattaggaaaaggctggatcagctgggataccgccagactctgacagtggagtgtttacctttggtagaaaaacttttcagcgacttagttcatacaactgagagccttcggcaatcaaaattatctgctgtgaaagctgaaaaagaaagtgccaattttgattttgttttggaaccctataaacttgaaaatgcaagattgagtagagaaaataatgaattatacctagagttaatgaaactgagagaacattcagaccaacacgttaaagagttgaaaacttcattgaagaaatgtgcacgtgaaacagctgatctgaaatttctgaataaccaatatgctcataaactcaaactgttggagaaagagagcaaagctaagaatgaaagaattcaacaacttcaagaaaagaatttgcatgctgtagtacaaactccaggtggcaagaaaagaagtattgctttcaggcgccagcgtatgcaaattgatgaaccggttcctccctctgaagtcagttcatatccagttcctcaaccagatgacccttacattgcagacctccttcaagtggctgataacaggattcaagaacttcaacaggaagtccaccagctacaagaaaagttagcaatgatggaaagtggggtgagagactatagcaagcagattgagctaagagaacgagagatagaacgactgtcagttgctttggatggtggtcggtcccctgatgtcctttctctggagtctagaaataaaaccaatgaaaagcttattgctcatttaaatattcaggttgactttcttcagcaagctaataaagacctggagaagcgtatacgagagcttatggaaaccaaggaaacagtgacatctgaagtcgttaatttaagtaacaaaaatgaaaaactctgccaagaattaactgaaatagatcagttagcacagcagttggaaagacataaagaagaagtgcttgagactgctgataaagagcttggggaagcaaagaaagagattaaaagaaagctctctgaaatgcaggatcttgaagaaacaatggcaaaacttcagctggaattgaacttatgccagaaagaaaaggagagactgagtgatgaactccttgtaaaatcagacctagaaactgttgttcatcagcttgaacaagaaaagcaaagacttagcaaaaaagttgaaagttttgcagttacagaacgacaacttactctggaggttgagaggatgagactagaacatggaataaaacgtcgagacaggtcaccttctcgtttagatacatttctgaaaggtatagaagaagaacgagattattataagaaagagctagagagactccaacatataatacagcgaagatcttgctctacaagttatagcgcacgtgaaaaaagttcaatatttagaacaccagaaaaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 14 - DNA, 2607 bp, synthetic construct
  source 1..2607: mol_type = other DNA
atgttaaggaggattttacagaggactcctgggagagttggctctcaaggttctgatttagattcatcagcaactcctataaacacagtggacgtcaataatgaaagctcttcagagggtttcatatgtccccagtgtatgaaatctcttggatctgctgatgaacttttcaaacattatgaagctgttcatgatgctggtaatgactcaggtcatggaggagagtctaatcttgctttgaagcgagatgatgtaacactgctcagacaagaggtccaagacctacaggcttcacttaaggaagaaaaatggtactcggaagaattaaagaaggaattagaaaaatatcaagggctgcagcagcaagaggccaaacctgatgggttggtgactgattcatcagcagaactacagtctttggaacagcaattagaagaagcccaaacagaaaattttaatattaagcaaatgaaagacttatttgaacagaaagcagcccaacttgctactgaaattgcagatataaagtcaaagtatgatgaagaaaggagtcttcgagaagctgctgaacaaaaagtgacacgtctgacagaagaattaaacaaagaggcaactgtaattcaagatctgaagacggaactgcttcagagacctggtatagaagatgttgccgtgctaaagaaagaactggtccaagttcaaacactaatggataacatgaccttggaacgtgagcgagaatctgaaaaactcaaagatgaatgcaaaaaattgcagtcacaatatgctagctcagaggccacaataagccagctaaggagtgaacttgccaaaggcccccaggaagttgctgtatatgtacaggaactacaaaaactgaaaagttcagttaatgaattaacacaaaaaaatcagaccttgacagaaaacttgctgaaaaaagaacaagactatactaagttagaggagaaacataatgaagaatctgtgagtaaaaagaatattcaggcaacccttcatcaaaaagacctagattgtcaacagcttcagtcaagattgtctgcatctgaaacctcactgcatagaatacatgtagaactaagtgaaaaaggagaagctactcaaaagctcaaagaagaattatctgaggtagagaccaagtaccagcatctaaaggcggagtttaagcagctacaacaacagagagaagaaaaggagcagcatgggttacaactccaaagtgaaattaatcaattacatagcaaacttctggagacagagcgccaactaggggaagctcatggtaggctgaaggaacagagacagctttcaagtgaaaagttgatggataaagaacaacaagtggctgatttacaactcaaactttctcggttagaagagcagttgaaggaaaaagttacaaattctacagaattgcagcatcaattagataaaacaaagcaacagcatcaagaacaacaggctcttcagcaaagcaccacggcaaaacttcgagaagctcaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 15 - DNA, 2337 bp, synthetic construct
  source 1..2337: mol_type = other DNA
atgcctagaagaggactgattcttcacacccggacccactggttgctgttgggccttgctttgctctgcagtttggtattatttatgtacctcctggaatgtgccccccagactgatggaaatgcatctcttcctggtgttgttggggaaaattatggtaaagagtattatcaagccctcctacaggaacaagaagaacattatcagaccagggcaaccagtctgaaacgccaaattgcccaactaaaacaagaattacaagaaatgagtgagaagatgcggtcactgcaagaaagaaggaatgtaggggctaatggcataggctatcagagcaacaaagagcaagcacctagtgatcttttagagtttcttcattcccaaattgacaaagctgaagttagcataggggccaaactacccagtgagtatggggtcattccctttgaaagttttaccttaatgaaagtatttcaattggaaatgggtctcactcgccatcctgaagaaaagccagttagaaaagacaaacgagatgaattggtggaagttattgaagcgggcttggaggtcattaataatcctgatgaagatgatgaacaagaagatgaggagggtccccttggagagaaactgatatttaatgaaaatgacttcgtagaaggttattatcgcactgagagagataagggcacacagtatgaactcttttttaagaaagcagaccttacggaatatagacatgtgaccctcttccgcccttttggacctctcatgaaagtgaagagtgagatgattgacatcactagatcaattattaatatcattgtgccacttgctgaaagaactgaagcatttgtacaatttatgcagaacttcagggatgtttgtattcatcaagacaagaagattcatctcacagtggtgtattttggtaaagaaggactgtctaaagtcaagtctatcctagaatctgtcaccagtgagtctaattttcacaattacaccttggtctcattgaatgaagaatttaatcgtggacgaggactaaatgtgggtgcccgagcttgggacaagggagaggtcttgatgtttttctgtgatgttgatatctatttctcagccgaattccttaacagctgccggttaaatgctgagccaggtaagaaggtgttttaccctgtggtgttcagtctttacaatcctgccattgtttatgccaaccaggaagtgccaccacctgtggagcagcagctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 16 - DNA, 4005 bp, synthetic construct
  source 1..4005: mol_type = other DNA
atggaagaaaatgaaagccagaaatgtgagccgtgccttccttactcagcagacagaagacagatgcaggaacaaggcaaaggcaatctgcatgtaacatcaccagaagatgcagaatgccgcagaaccaaggaacgcctttctaatggaaacagtcgtggttcagtttccaagtcttcccgcaatatcccaaggagacacaccctaggggggccccgaagttccaaggaaatactgggaatgcaaacatctgagatggatcggaagagagaagcgttcctagaacatctgaagcagaagtacccccaccacgcctctgcaatcatgggtcaccaagagaggctgagagaccagacaaggagccccaaactgtctcacagtcctcaaccacccagtctgggtgacccggtcgagcatttatcagagacgtccgctgattctttggaagccatgtctgagggggatgctccaacccctttttccagaggcagccggactcgtgcgagccttcctgtggtgaggtcaaccaaccagacgaaagaaagatctctgggggttctctatctccagtatggagatgaaaccaagcagctcaggatgccgaatgaaatcacaagtgcagacacaatccgtgctctcttcgtaagtgcctttccacagcagctcaccatgaaaatgctggaatcgcccagtgtcgccatttacatcaaagatgaaagcagaaatgtctattatgaattaaatgatgtaaggaacattcaagacagatcactcctcaaagtgtacaacaaggatcctgcacatgcgtttaatcacacaccaaaaactatgaatggagacatgaggatgcagagagaacttgtttatgcaagaggagatggccctggggcccctcgccccggatctactgctcatccaccccatgcgattccaaattccccaccgtctactccagtgccccattccatgcccccctccccgtccagaattccttatgggggcacccgctccatggttgttcctggcaatgccaccatccccagggacagaatctccagcctgccagtctccagacccatctctccaagcccaagcgccattttagaaagaagagatgtcaagcctgatgaagacatgagtggcaaaaacattgcaatgtacagaaatgagggtttctatgctgatccttacctttatcacgagggacggatgagcatagcctcatcccatggtggacacccactggatgtccccgaccacatcattgcatatcaccgcaccgccatccggtcagcgagtgcttattgtaacccctcaatgcaagcggaaatgcatatggaacaatcactgtacagacagaaatcaaggaaatatccggatagccatttgcctacactgggctccaaaacaccccctgcctctcctcacagagtcagtgacctgaggatgatagacatgcacgctcactataatgcccacggcccccctcacaccatgcagccagaccgggcctctccgagccgccaggcctttaaaaaggagccaggcaccttggtgtatatagaaaagccacggagcgctgcaggattatccagccttgtagacctcggccctcctctaatggagaagcaagtttttgcctacagcacggcgacaatacccaaagacagagagaccagagagaggatgcaagccatggagaaacagattgccagtttaactggccttgttcagtctgcgctttttaaagggcccattacaagttatagcaaagatgcgtctagcgagaaaatgatgaaaaccacagccaacaggaaccacacagatagtgcaggaacgccccatgtgtctggtgggaagatgctcagtgctctggagtccacggtgcctcccagccagcctccacctgtgggcacctcagccatccacatgagcctgcttgagatgaggcggagcgtggcggaactcaggctccagctccagcagatgcggcagctccagctgcagaaccaggagttgctgagggcaatgatgaagaaggccgagctggaaatcagtggcaaagtgatggaaacaatgaagagactggaggatcccgtgcagcgacagcgcgtcctagtggagcaagagagacaaaaatatcttcatgaggaagagaagatcgtcaagaagttgtgcgagttggaagactttgttgaagacttgaagaaggactccacggcagccagccgattggttactctgaaagacgtggaagacggggctttcctcctgcgtcaagtgggagaggctgtagctaccctgaaagatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 17 - DNA, 3771 bp, synthetic construct
  source 1..3771: mol_type = other DNA
atgagttctgaatgtgatggtggttccaaagctgtgatgaatggcttggcacctggcagcaatgggcaagacaaagacatggatcttacaaaaatctgcactgggaagggagcggtgactctccgggcctcgtcttcctacagggaaaccccaagcagtagccctgcgagccctcaggaaacccggcaacacgaaagcaaaccagatgagtggaggctttcttccagtgctgatgccaatggaaatgcccagccctcttcactcgctgccaagggctacagaagtgtgcatcccaaccttccttctgacaagtcccaggattccagtcctctactaaatgaagtttcttcttcccttattggaactgattcccaagcctttccatcagttagcaagccttcatccgcctatccctccacaacgattgtcaatcctactattgtgctcttgcaacacaatcgagaacagcaaaaacgactcagtagcctttcagatcctgtctcagaaagaagagtgggagagcaggactcagcaccaacccaggaaaaacccacctcacctggcaaggctattgaaaaaagagcaaaggatgacagtaggcgggtggtgaagagcactcaggacttaagcgatgtttccatggatgaagtgggcatcccactccggaacactgagagatcaaaagactggtacaagactatgtttaaacagatccacaaactgaacagagatgatgattcagatctgtactctcccagatactcattttctgaagacacaaaatctcccctttctgtgcctcgctcaaaaagtgagatgagctacattgatggtgagaaggtagtcaagaggtcggccacactacccctcccagcccgctcttcctcactgaagtcaagctcagaaagaaatgactgggaacccccagataagaaagtagacacaagaaaatatcgtgcagagcccaagagcatttacgaatatcagcctggcaagtcttccgttctgaccaacgaaaagatgagctcagccatcagccctactccggaaatttcttcagagactcctggatatatatattcttccaacttccatgcagtgaagagggaatcagacggggctcctggggatctcactagcttggagaatgagagacaaatttataaaagtgtcttggaaggtggtgacatccctcttcagggcctgagtgggctcaagcgaccatccagctctgcttccactaaagattcagaatcgccaagacattttataccagctgattacttggaatccacggaagaatttattcgaagacgtcatgatgataaagagatgagacctgccagagccaaatttgactttaaagctcagacactaaaggagcttcctctgcagaagggagatattgtttacatttataagcaaattgatcagaactggtatgaaggagaacaccacggccgggtgggaatcttcccacgcacctacatcgagcttcttcctcctgctgagaaggcacagcccaaaaagttgacaccagtgcaggttttggaatatggagaagctattgctaagtttaactttaatggtgatacacaagtagaaatgtccttcagaaagggtgagaggatcacactgctccggcaggtagatgagaactggtacgaagggaggatcccggggacatcccgacaaggcatcttccccatcacctacgtggatgtgatcaagcgaccactggtgaaaaaccctgtggattacatggacctgcctttctcctcctccccaagtcgcagtgccactgcaagcccacagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 18 - DNA, 3327 bp, synthetic construct
  source 1..3327: mol_type = other DNA
atgagttctgaatgtgatggtggttccaaagctgtgatgaatggcttggcacctggcagcaatgggcaagacaaagacatggatcttacaaaaatctgcactgggaagggagcggtgactctccgggcctcgtcttcctacagggaaaccccaagcagtagccctgcgagccctcaggaaacccggcaacacgaaagcaaaccagatgagtggaggctttcttccagtgctgatgccaatggaaatgcccagccctcttcactcgctgccaagggctacagaagtgtgcatcccaaccttccttctgacaagtcccaggattccagtcctctactaaatgaagtttcttcttcccttattggaactgattcccaagcctttccatcagttagcaagccttcatccgcctatccctccacaacgattgtcaatcctactattgtgctcttgcaacacaatcgagaacagcaaaaacgactcagtagcctttcagatcctgtctcagaaagaagagtgggagagcaggactcagcaccaacccaggaaaaacccacctcacctggcaaggctattgaaaaaagagcaaaggatgacagtaggcgggtggtgaagagcactcaggacttaagcgatgtttccatggatgaagtgggcatcccactccggaacactgagagatcaaaagactggtacaagactatgtttaaacagatccacaaactgaacagagatgatgattcagatctgtactctcccagatactcattttctgaagacacaaaatctcccctttctgtgcctcgctcaaaaagtgagatgagctacattgatggtgagaaggtagtcaagaggtcggccacactacccctcccagcccgctcttcctcactgaagtcaagctcagaaagaaatgactgggaacccccagataagaaagtagacacaagaaaatatcgtgcagagcccaagagcatttacgaatatcagcctggcaagtcttccgttctgaccaacgaaaagatgagctcagccatcagccctactccggaaatttcttcagagactcctggatatatatattcttccaacttccatgcagtgaagagggaatcagacggggctcctggggatctcactagcttggagaatgagagacaaatttataaaagtgtcttggaaggtggtgacatccctcttcagggcctgagtgggctcaagcgaccatccagctctgcttccactaaagattcagaatcgccaagacattttataccagctgattacttggaatccacggaagaatttattcgaagacgtcatgatgataaagagatgagacctgccagagccaaatttgactttaaagctcagacactaaaggagcttcctctgcagaagggagatattgtttacatttataagcaaattgatcagaactggtatgaaggagaacaccacggccgggtgggaatcttcccacgcacctacatcgagcttcttcctcctgctgagaaggcacagcccaaaaagttgacaccagtgcaggttttggaatatggagaagctattgctaagtttaactttaatggtgatacacaagtagaaatgtccttcagaaagggtgagaggatcacactgctccggcaggtagatgagaactggtacgaagggaggatcccggggacatcccgacaaggcatcttccccatcacctacgtggatgtgatcaagcgaccactggtgaaaaaccctgtggattacatggacctgcctttctcctcctccccaagtcgcagtgccactgcaagcccacagcaacctcaagcccagcagcgaagagtcacccccgacaggagtcaaacctcacaagatttatttagctatcaagcattatatagctatataccacagaatgatgatgagttggaactccgcgatggagatatcgttgatgtcatggaaaaatgtgacgatggatggtttgttgatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 19 - DNA, 4128 bp, synthetic construct
  source 1..4128: mol_type = other DNA
atgtcggcagccaaggagaacccgtgcaggaaattccaggccaacatcttcaacaagagcaagtgtcagaactgcttcaagccccgcgagtcgcatctgctcaacgacgaggacctgacgcaggcaaaacccatttatggcggttggctgctcctggctccagatgggaccgactttgacaacccagtgcaccggtctcggaaatggcagcgacggttcttcatcctttacgagcacggcctcttgcgctacgccctggatgagatgcccacgacccttcctcagggcaccatcaacatgaaccagtgcacagatgtggtggatggggagggccgcacgggccagaagttctccctgtgtattctgacgcctgagaaggagcatttcatccgggcggagaccaaggagatcgtcagtgggtggctggagatgctcatggtctatccccggaccaacaagcagaatcagaagaagaaacggaaagtggagccccccacaccacaggagcctgggcctgccaaggtggctgttaccagcagcagcagcagcagcagcagcagcagcagcatccccagtgctgagaaagtccccaccaccaagtccacactctggcaggaagaaatgaggaccaaggaccagccagatggcagcagcctgagtccagctcagagtcccagccagagccagcctcctgctgccagctccctgcgggaacctgggctagagagcaaagaagaggagagcgccatgagtagcgaccgcatggactgtggccgcaaagtccgggtggagagcggctacttctctctggagaagaccaaacaggacttgaaggctgaagaacagcagctgcccccgccgctctcccctcccagccccagcacccccaaccacaggaggtcccaggtgattgaaaagtttgaggccttggacattgagaaggcagagcacatggagaccaatgcagtggggccctcaccatccagcgacacacgccagggccgcagcgagaagagggcgttccctaggaagcgggacttcaccaatgaagcccccccagctcctctcccagacgcctcggcttcccccctgtctccacaccgaagagccaagtcactggacaggaggtccacggagccctccgtgacgcccgacctgctgaatttcaagaaaggctggctgactaagcagtatgaggacggccagtggaagaaacactggtttgtcctcgccgatcaaagcctgagatactacagggattcagtggctgaggaggcagccgacttggatggagaaattgacttgtccgcatgttacgatgtcacagagtatccagttcagagaaactatggcttccagatacatacaaaggagggcgagtttaccctgtcggccatgacatctgggattcggcggaactggatccagaccatcatgaagcacgtgcacccgaccactgccccggatgtgaccagctcgttgccagaggaaaaaaacaagagcagctgctcttttgagacctgcccgaggcctactgagaagcaagaggcagagctgggggagccggaccctgagcagaagaggagccgcgcacgggagcggaggcgagagggccgctccaagacctttgactgggctgagttccgtcccatccagcaggccctggctcaggagcgggtgggcggcgtggggcctgctgacacccacgagcccctgcgccctgaggcggagcctggggagctggagcgggagcgtgcacggaggcgggaggagcgccgcaagcgcttcgggatgctcgacgccacagacgggccaggcactgaggatgcagccctgcgcatggaggtggaccggagcccagggctgcctatgagcgacctcaaaacgcataacgtccacgtggagattgagcagcggtggcatcaggtggagaccacacctctccgggaagagaagcaggtgcccatcgcccccgtccacctgtcttctgaagatgggggtgaccggctctccacacacgagctgacctctctgctcgagaaggagctggagcagagccagaaggaggcctcagaccttctggagcagaaccggctcctgcaggaccagctgagggtggccctgggccgggagcagagcgcccgtgagggctacgtgctgcaggccacgtgcgagcgagggtttgcagcaatggaagaaacgcaccagaagaagattgaagatctccagaggcagcaccagcgggagctagagaaacttcgagaagagaaagaccgcctcctagccgaggagacagcggccaccatctcagccatcgaagccatgaagaacgcccaccgggaggaaatggagcgggagctggagaagagccagcggtcccagatcagcagcgtcaactcggatgttgaggccctgcggcgccagtacctggaggagctgcagtcggtgcagcgggaactggaggtcctctcggagcagtactcgcagaagtgcctggagaatgcccatctggcccaggcgctggaggccgagcggcaggccctgcggcagtgccagcgtgagaaccaggagctcaatgcccacaaccaggagctgaacaaccgcctggctgcagagatcacacggttgcggacgctgctgactggggacggcggtggggaggccactgggtcaccccttgcacagggcaaggatgcctatgaactagaggtcttattgcgggtaaaggaatcggaaatacagtacctgaaacaggagattagctccctcaaggatgagctgcagacggcactgcgggacaagaagtacgcaagtgacaagtacaaagacatctacacagagctcagcatcgcgaaggctaaggctgactgtgacatcagcaggttgaaggagcagctcaaggctgcaacggaagcactgggggagaagtcccctgacagtgccacggtgtccggatatgatataatgaaatctaaaagcaaccctgacttcttgaagaaagacagatcctgtgtcacccggcaactcagaaacatcaggtccaaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 20 - DNA, 1920 bp, synthetic construct
  source 1..1920: mol_type = other DNA
atgaacggacagttggatctaagtgggaagctaatcatcaaagctcaacttggggaggatattcggcgaattcctattcataatgaagatattacttatgatgaattagtgctaatgatgcaacgagttttcagaggaaaacttctgagtaatgatgaagtaacaataaagtataaagatgaagatggagatcttataacaatttttgatagttctgacctttcctttgcaattcagtgcagtaggatactgaaactgacattatttgttaatggccagccaagaccccttgaatcaagtcaggtgaaatatctccgtcgagaactgatagaacttcgaaataaagtgaatcgtttattggatagcttggaaccacctggagaaccaggaccttccaccaatattcctgaaaatgatactgtggatggtagggaagaaaagtctgcttctgattcttctggaaaacagtctactcaggttatggcagcaagtatgtctgcttttgatcctttaaaaaaccaagatgaaatcaataaaaatgttatgtcagcgtttggcttaacagatgatcaggtttcagatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 21 - DNA, 3735 bp, synthetic construct
  source 1..3735: mol_type = other DNA
atgaagaaagcaagcaggagtgttggctcagtgcctaaagtgtctgcaataagtaaaacgcaaacagcagaaaaaattaaacctgaaaacagctcttcagcatctacgggaggcaaacttgtaaaacctggaacagcagcatcattgtcaaagaccaagagcagtgatgaccttttagctggaatggccggaggggtaacggtgactaatggtgttaaaggaaagaaaagcacctgcccatctgcagcaccttcagcatctgcccctgccatgaccaccgtggagaacaaatccaagattagcacaggcacagcttcttcaaccaagcggagcacttctacaggtaataaagaatccagttctactagagaaagattacgtgaacgtacccgattaaaccagagcaaaaaactaccttctgcaggtcagggagctaatgacatggcattggccaaacgttcccgcagtcgaactgctacagaatgtgacgttcgtatgagcaagtctaagtcagacaatcagatcagtgacagagctgctttggaggccaaagtgaaggatcttctcacgctggcaaaaaccaaagacgtagaaattttacatttgagaaatgaactgcgagacatgcgtgcccagctgggcattaatgaggatcattctgagggtgatgaaaaatctgagaaggaaactattatggctcaccagccgactgatgtggagtccactttattgcagttgcaggaacagaatactgccatccgtgaagaactcaaccagctgaaaaatgaaaacagaatgttaaaggacaggttgaatgcattgggcttttccctagagcagaggttagacaattctgaaaaactgtttggctatcagtccctgagcccagaaatcacccctggtaaccagagcgatggaggaggaactctgacttcttcagtggaaggctctgcccctggctcagtggaggatctcttgagtcaggatgaaaatacactaatggaccatcagcacagtaactccatggacaatttagacagtgagtgcagtgaggtctaccagcccctcacatcgagcgatgatgcgctggatgcaccatcctcctcagagtcggaaggcatccccagcatagagcgctcccggaaggggagcagcgggaatgccagtgaagtgtccgtggcttgcctgactgaacggatacaccagatggaagagaaccaacacagtacaagtgaggaactccaggcaaccctgcaagagctagctgatttacagcagattacccaggaactgaatagtgaaaacgaaaggcttggagaagagaaggttattctgatggagtctttatgtcagcagagcgataagttggaacactttagtcgacagattgaatacttccgctctcttctagatgagcatcacatttcttatgtcatagatgaagatgtaaaaagtgggcgctatatggaattagagcaacgttacatggacctcgctgagaatgcccgttttgaacgggagcagcttcttggtgtccagcagcatttaagcaatactttgaaaatggcagaacaagacaataaggaagctcaagaaatgataggggcactcaaagaacgcagtcaccatatggagcgaattattgagtctgagcagaaaggaaaagcagccttggcagccacgttagaggaatacaaagccacagtggccagtgaccagatagagatgaatcgcctgaaggctcagctggagaatgaaaagcagaaagtggcagagctgtattctatccataactctggagacaaatctgatattcaggacctcctggagagtgtcaggctggacaaagaaaaagcagagactttggctagtagcttgcaggaagatctggctcatacccgaaatgatgccaatcgattacaggatgccattgctaaggtagaggatgaataccgagccttccaagaagaagctaagaaacaaattgaagatttgaatatgacgttagaaaaattaagatcagacctggatgaaaaagaaacagaaaggagtgacatgaaagaaaccatctttgaacttgaagatgaagtagaacaacatcgtgctgtgaaacttcatgacaacctcattatttctgatctagagaatacagttaaaaaactccaggaccaaaagcacgacatggaaagagaaataaagacactccacagaagacttcgggaagaatctgcggaatggcggcagtttcaggctgatctccagactgcagtagtcattgcaaatgacattaaatctgaagcccaagaggagattggtgatctaaagcgccggttacatgaggctcaagaaaaaaatgagaaactcacaaaagaattggaggaaataaagtcacgcaagcaagaggaggagcgaggccgggtatacaattacatgaatgccgttgagagagatttggcagccttaaggcagggaatgggactgagtagaaggtcctcgacttcctcagagccaactcctacagtaaaaaccctcatcaagtcctttgacagtgcatctcaagtaccaaaccctgctgcagctgcaattcctcgaacgcccctgagcccaagtcctatgaaaacccctcctgcagcagctgtgtcccctatgcaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 22 - DNA, 1500 bp, synthetic construct
  source 1..1500: mol_type = other DNA
atggtgtcctggatgatctccagagccgtggtgctggtgtttggaatgctttatcctgcatattattcatacaaagctgtgaaaacaaaaaacgtgaaggaatatgttcgatggatgatgtactggattgtttttgctctctatactgtgattgaaacagtagccgatcaaacagttgcttggtttcccctgtactatgagctgaagattgcttttgtcatatggctgctttctccctataccaaaggagcaagtttaatatatagaaaattccttcatccacttctttcttcaaaggaaagggagattgatgattatattgtacaagcaaaggaacgaggctatgaaaccatggtaaactttggacggcaaggtttaaaccttgcagctactgctgctgttactgcagcagtaaaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 23 - DNA, 3735 bp, synthetic construct
  source 1..3735: mol_type = other DNA
atggatatttacgacactcaaaccttgggggttgtggtctttggaggattcatggttgtttctgccattggcatcttcctggtgtcgactttctccatgaaggaaacgtcatatgaagaagccctagccaaccagcgcaaggagatggcgaaaactcaccaccagaaagtcgagaagaaaaagaaggagaaaacagtggagaagaaaggaaagaccaagaaaaaggaagagaaacctaatgggaagatacctgatcatgatccagcccccaatgtgactgtcctccttcgagaaccagtgcgggctcctgctgtggctgtggctccaaccccagtgcagccccccattatcgttgctcctgtcgccacagttccagccatgccccaggagaagctggcctcctcccccaaggacaaaaagaagaaggagaaaaaagtggcaaaagtggaaccagctgtcagctctgtagtgaattccatccaggttctcacttcgaaggctgccatcttggaaactgctcccaaggagggcagaaatacagatgtggcccagagcccagaggcaccaaagcaagaggctcctgccaagaagaagtctggttcaaagaaaaaagggcccccagatgccgacggccctctctacctcccctacaagacgctggtctccacggttgggagcatggtgttcaacgagggcgaggcccagcggctcatcgagatcctgtctgagaaggctggcatcattcaggacacctggcacaaggccactcagaagggtgaccctgtggcgattctgaaacgccagctggaagagaaggaaaaactgctggccacagaacaggaagatgcggctgtcgccaagagcaaactgagggagctcaacaaggagatggcagcagaaaaggccaaagcagcagccggggaggccaaagtgaaaaagcagctggtggcccgggagcaggagatcacggctgtgcaggcacgcatgcaggccagctaccgggagcacgtgaaggaggtgcagcagctgcagggcaagatccggactcttcaggagcagctggagaatggccccaacacgcagctggcccgcctgcagcaggagaactccatcctgcgggatgccttgaaccaggccacgagccaggtggagagcaagcagaacgcagagctggccaagcttcggcaggagctcagcaaggtcagcaaagagctggtggagaagtcagaggctgtgcggcaagatgagcagcagcggaaagctctggaagccaaggcagctgccttcgagaagcaggtcctgcagctgcaggcgtcccacagggagagtgaggaggccctgcagaagcgcctggacgaggtcagccgggagctgtgccacacgcagagcagccacgccagcctccgggcggatgccgagaaggcccaggagcaacagcagcagatggccgagctgcacagcaagttacagtcctccgaggcggaggtgcgcagcaaatgcgaggagctgagtggcctccacgggcagctccaggaggccagggcggagaactcccagctcacagagagaatccgttccattgaggccctgctggaggcgggccaggcgcgggatgcccaggacgtccaggccagccaggcggaggctgaccagcagcagactcgcctcaaggagctggagtcccaggtgtcgggtctggagaaggaggccatcgagctcagggaggccgtcgagcagcagaaagtgaagaacaatgacctccgggagaagaactggaaggccatggaggcactggccacggccgagcaggcctgcaaggagaagctgctctccctgacccaggccaaggaggaatcggagaagcagctctgtctgattgaggcgcagaccatggaggccctgctggctctgctcccagaactctctgtcttggcacaacagaattacaccgagtggctgcaggatctcaaagagaaaggccccacgctgctgaagcacccgccagctcccgcggagccctcctcggacctggcctccaagttgagggaggccgaggagacgcagagcacactgcaggccgagtgtgaccagtaccgcagcatcctggcggagacggagggcatgctcagagacctgcagaagagcgtggaggaggaggagcaggtgtggagggccaaggtgggcgccgcagaggaggagctccagaagtcccgggtcacagtgaagcatctcgaagagattgtagagaagctaaaaggagaacttgaaagttcggaccaggtgagggagcacacgtcgcatttggaggcagagctggaaaagcacatggcggccgccagcgccgagtgccagaactacgccaaggaggtggcagggctgaggcaacttctcctagaatctcaatctcagctcgatgccgccaagagcgaagcccagaaacagagcgatgagcttgccctggtcaggcagcagttgagtgaaatgaagagccacgtagaggatggtgacatagctggggccccagcttcctccccagaggcgcccccagccgagcaggaccccgttcagctgaagacgcagctggagtggacagaagccatcctggaggatgagcagacacagcggcagaagctcacggccgagtttgaggaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 24 - DNA, 2235 bp, synthetic construct
  source 1..2235: mol_type = other DNA
atgtctgagactcctgctcagtgtagcattaagcaggaacgaatttcatatacacctccagagagcccagtgccgagttacgcttcctcgacgccacttcatgttccagtgcctcgagcgctcaggatggaggaagactcgatccgcctgcctgcgcacctgcgcttgcagccaatttactggagcagggatgacgtagcccagtggctcaagtgggctgaaaatgagttttctttaaggccaattgacagcaacacgtttgaaatgaatggcaaagctctcctgctgctgaccaaagaggactttcgctatcgatctcctcattcaggtgatgtgctctatgaactccttcagcatattctgaagcagaggaaacctcggattcttttttcaccattcttccaccctggaaactctatacacacacagccggaggtcatactgcatcagaaccatgaagaagataactgtgtccagaggacccccaggccatccgtggataatgtgcaccataaccctcccaccattgaactgttgcaccgctccaggtcacctatcacgacaaatcaccggccttctcctgaccccgagcagcggcccctccggtcccccctggacaacatgatccgccgcctctccccggctgagagagctcagggacccaggccgcaccaggagaacaaccaccaggagtcctaccctctgtcagtgtctcccatggagaataatcactgcccagcgtcctccgagtcccacccgaagccatccagcccccggcaggagagcacacgcgtgatccagctgatgcccagccccatcatgcaccctctgatcctgaacccccggcactccgtggatttcaaacagtccaggctctccgaggacgggctgcatagggaagggaagcccatcaacctctctcatcgggaagacctggcttacatgaaccacatcatggtctctgtctccccgcctgaagagcacgccatgcccattgggagaatagcagactgtagactgctttgggattacgtctatcagttgctttctgacagccggtacgaaaacttcatccgatgggaggacaaagaatccaaaatattccggatagtggatcccaacggactggctcgactgtggggaaaccataaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 25 - DNA, 3315 bp, synthetic construct
  source 1..3315: mol_type = other DNA
atgtgcgagagttactccaggtcgttgttgagggtctcggtggcgcagatctgccaggcgctgggctgggactcggtgcagctcagcgcctgccacctcctcacggacgtgctgcagcgctatctgcagcagctgggccggggctgccatcggtactctgagctctatggccgaacagacccaattttggatgatgttggtgaagctttccagctgatgggggttagtctacatgaactagaagactatattcacaacattgagcctgtcaccttcccacaccaaattccgtcatttcctgttagcaagaacaatgtacttcagtttcctcaacctggaagtaaagatgcagaggaaagaaaagaatacattcctgattacctgccacccattgtgtcttctcaagaagaagaagaagaagagcaggtgcccactgatggaggcacatcagcagaagccatgcaggttcccttggaagaagatgatgaattggaggaggaagaaattattaatgatgagaatttcctgggcaagagaccactggatagtcctgaagctgaagaactgccagccatgaagcggcctcggctattaagcactaaaggggacacgctagatgttgtgttattggaagctcgagagccactcagctcaataaatactcaaaagatcccaccaatgctttctccagtccatgtacaggacagtacagacttggcacctccctcacccgagccgccaatgttggctccagttgcaaaatcacaaatgccaactgcaaaaccattagaaacaaagtcatttacacctaaaacaaagactaaaactagctctccaggacagaagactaaatcacctaaaaccgcccagtcaccagcaatggtcggaagtcctattcgatcaccaaaaactgtatccaaagaaaagaaatcacctggacgttccaagagccccaagagtcccaagagccccaaggtcacgactcacattccccaaacacctgtgagacctgaaacgcccaacaggactccttcagctacactcagtgaaaaaatcagtaaagagactatccaggtaaaacaaatacagacaccccctgatgctgggaaactgaacagtgagaatcagccgaaaaaggctgtggtagcagataaaacgattgaggcctctatcgatgctgtgattgcacgagcctgtgctgagcgagagccagatcctttcgaattttcttctggatcggaatctgaaggagacatttttactagccctaagagaatttcaggcccggagtgtactactcccaaagcttccacttccgcgaacaatttcacaaagtcaggatccactcctctgcctctttccggtggaacctcaagttccgataactcatggacaatggatgcctccattgatgaggttgtacgtaaagcaaaactgggaacaccttcaaatatgccccccaactttccttatatctcttctccgtcagtgtctcctcccactcccgaacctctccacaaggtgtatgaggagaaaaccaagctgccttcctccgtggaggtaaagaagaagttgaaaaaggaactaaagactaaaatgaaaaagaaagaaaagcagagagatagggagagggaaaaagacaagaacaaggacaaaagtaaggagaaggataaagtgaaagagaaagagaaagacaaggaaactggcagggaaacaaagtatccctggaaggaatttcttaaagaggaagaggcagatccctacaagtttaaaatcaaagaatttgaagatgttgatcccaaagtgaaattgaaagatggacttgtgaggaaggagaaagagaagcataaagataagaagaaagatagagagaaaggcaagaaagataaagataagagagagaaagaaaaagtgaaagataaaggcagagaagataagatgaaagccccagcacccccactggtgttgcccccaaaagagttggccctgcccttgttcagccctgccacagcctccagggtcccagccatgctgccatctttgttgccagtgcttccggaaaaactgtttgaggagaaagagaaggtgaaggagaaagaaaagaaaaaggacaaaaaggagaagaagaaaaagaaggaaaaagagaaggagaagaaggagaaggaaagagagaaagagaagagagagcgagagaagagagaaaaagagaaggagaaacacaagcatgaaaaagaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 26 - DNA, 5910 bp, synthetic construct
  source 1..5910: mol_type = other DNA
atggccacaggaggaggtccctttgaagatggcatgaatgatcaggatttaccaaactggagtaatgagaatgttgatgacaggctcaacaatatggattggggtgcccaacagaagaaagcaaatagatcatcagaaaagaataagaaaaagtttggtgtagaaagtgataaaagagtaaccaatgatatttctccggagtcgtcaccaggagttggaaggcgaagaacaaagactccacatacgttcccacacagtagatacatgagtcagatgtctgtcccagagcaggcagaattagagaaactgaaacagcggataaacttcagtgatttagatcagagaagcattggaagtgattcccaaggtagagcaacagctgctaacaacaaacgtcagcttagtgaaaaccgaaagcccttcaactttttgcctatgcagattaatactaacaagagcaaagatgcatctacaaaccccccaaacagagaaacgattggatcagcacagtgtaaagagttgtttgcttctgctttaagtaatgacctcttgcaaaactgtcaggtgtctgaagaagatgggaggggagaacctgcaatggagagcagccagattgtaagcaggcttgttcaaattcgcgattatattactaaagctagttccatgcgggaagatcttgtagagaaaaatgagagatctgctaatgttgagcgccttactcatctaatagatcaccttaaagaacaagagaagtcatatatgaaatttcttaaaaaaatccttgccagagatcctcagcaggagcctatggaagagatagaaaatttgaagaaacaacatgatttattaaaaagaatgttacaacagcaggagcaactaagagctctacagggacggcaggctgcacttctagctctgcaacataaagcagagcaagctattgcagtgatggatgattctgttgttgcagaaactgcaggtagcttatctggcgtcagtatcacatctgaactaaatgaagaattgaatgacttaattcagcgttttcataatcagcttcgtgattctcagcctccagctgttccagacaatagaagacaggcagaaagtctttcattaactagggaggtttcccagagcaggaaaccatcagcttcagaacgtttacctgatgagaaagtcgaactttttagcaaaatgagagtgctacaggaaaagaaacaaaaaatggacaaattgcttggagaacttcatacacttcgagatcagcatcttaacaattcatcatcctctccacaaaggagtgtcgatcagagaagtacttcagctccctctgcttctgtaggcttggcaccggttgtcaatggagaatccaatagcctcacatcatctgttccttatcctactgcttctctagtatctcagaatgagagtgaaaacgaaggccacctcaatccatctgaaaaactccagaagttaaatgaagttcgaaagagattgaatgagctaagagaattagttcattattatgaacaaacgtcagacatgatgacagatgctgtgaatgaaaacaggaaagatgaagaaactgaagagtcagaatatgattctgagcatgaaaattccgagcctgttactaacattcgaaatccacaagtagcttccacttggaatgaagtaaatagtcatagtaatgcacagtgtgtttctaataatagagatgggcgaacagttaattctaattgtgaaattaacaacagatctgctgccaacataagggctctaaacatgcctccttctttagattgtcgatataatagagaaggggaacaggagattcatgttgcacaaggtgaagatgatgaggaggaggaggaagaagcagaagaggagggagtcagtggagcttcattatctagtcacaggagcagtctggttgatgagcatccagaagatgctgaatttgaacagaagatcaaccgacttatggctgcaaaacagaaacttagacagttacaagatcttgttgctatggtacaggatgatgatgcagctcaaggagttatctctgccagtgcatcaaatttggatgatttctacccagcagaagaagacaccaagcaaaattcaaataacactagaggaaatgccaataaaacacagaaagatactggagtaaatgaaaaggcaagagagaaattttatgaggctaaactacagcagcaacagagagagctaaaacaattgcaggaagaaagaaagaaactgattgacattcaggagaaaattcaagcattgcaaacggcatgccctgacttacagctgtcagctgctagtgtgggtaactgtcccaccaaaaaatatatgccagctgttacttcaaccccaactgttaatcaacacgagaccagtacaagcaaatctgtttttgagcctgaagattcttcaatagtagataatgagttgtggtcagaaatgagaagacatgaaatgttgagggaggagctgcgacagagaagaaagcagcttgaagctctgatggctgaacatcagaggaggcaaggtctagctgaaactgcatctccagtggctgtgtcattgagaagtgatggatctgagaacctatgtactcctcagcaaagtagaacagaaaaaacgatggcaacttggggagggtctacccagtgtgcactagatgaagaaggagatgaagacggttacctttctgaaggaattgttcggacagatgaagaggaggaagaagagcaagatgccagttccaatgataacttttctgtgtgtccttctaacagtgtgaatcataactcctacaatggaaaggaaactaaaaataggtggaagaacaattgccctttttcggcagatgaaaattatcgtcctttagccaagacaaggcaacagaatatcagcatgcaacggcaagaaaaccttcgttgggtgtcagagctctcttacgtagaagagaaagaacaatggcaagaacaaatcaatcagctaaagaaacagcttgattttagtgtcagtatttgtcagactttgatgcaagaccagcagactctatcttgtctgctacaaactcttctcacgggtccttacagtgttatgcccagcaatgttgcatctcctcaagtacacttcataatgcaccagttgaaccagtgctatactcagctaacatggcaacagaataatgttcagaggttgaaacaaatgctaaatgaacttatgcgccagcaaaatcagcatccagaaaaacctggaggcaaggaaagaggcagtagtgcatcgcaccctccttctcccagtttattttgtcctttcagctttccaacacagcctgtaaatctcttcaatatacctggatttactaacttttcatcatttgcaccaggtatgaatttcagccctttatttccttctaattttggagatttttctcagaatatctctacacccagtgaacagcagcaacccttagcccagaattcttcaggaaaaacagaatatatggcttttccaaaaccttttgaaagcagttcctctattggagcagagaaaccaaggaataaaaaactgcctgaagaggaggtggaaagcagtaggacaccatggttatatgaacaagaaggtgaagtagagaaaccatttatcaagactggattttcagtgtctgtagaaaaatctacaagtagtaaccgcaaaaatcaattagatacaaacggaagaagacgccagtttgatgaagaatcactggaaagctttagcagtatgcctgatccagtagatccaacaacagtgactaaaacattcaagacaagaaaagcgtctgcacaggccagcctggcatctaaagataaaactcccaagtcaaaaagtaagaagaggaattctactcagctgaaaagcagagttaaaaacatcaggtatgaaagtgccagtatgtctagcacatgtgaaccttgcaaaagtaggaacagacattcagcccagactgaagagcctgttcaagcaaaagtattcagcagaaagaatcatgagcaactggaaaaaataataaaatgtaataggtctacagaaatatcttcagaaactgggagtgatttttccatgtttgaagctttgcgagatactatttattctgaagtagctacattaatttctcaaaatgaatctcgtccacattttcttattgaactcttccatgagctgcagctactaaacacagactacttgagacagagggctttatatgcattgcaggacatagtatccagacatatttctgagagccatgaaaaaggagaaaatgtaaagtcagtaaactctggtacttggatagcatcaaactcagaacttactcctagtgagagccttgctactactgatgatgaaacttttgagaagaactttgaaagagaaacccataaaataagtgagcaaaatgatgctgataatgctagtgtcctgtctgtatcatcaaattttgagccttttgcaacagatgatctaggtaacaccgtgattcacttagatcaagcattagccagaatgagagaatatgagcgtatgaagactgaggctgaaagtaactcaaatatgagatgcacctgcaggattattgaggatggagatggtgctggtgcaggtactacagttaataatttagaagaaactcccgttattgaaaatcgtagttcacaacaacctgtaagtgaagtttctaccatcccatgtcctagaattgatactcagcagctggaccggcaaattaaagcaattatgaaagaagtcattccttttttgaaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 27 - DNA, 5910 bp, synthetic construct; identical to SEQ ID NO: 26
  source 1..5910: mol_type = other DNA

SEQ ID NO: 28 - DNA, 2109 bp, synthetic construct
  source 1..2109: mol_type = other DNA
atgtcccgggacccggggtcgggcggctgggaggaggccccgcgcgcagctgccgcgctctgcaccctgtaccacgaggccggacagcggctgcgccgcctgcaggaccagctcgctgcccgcgacgccctcatcgctcgcctccgcgcccgcctggccgcgctggagggggacgccgcgccgtccctagtggacgcgctgctggagcaggttgcgcgcttccgggagcagctgcgaaggcaggagggcggcgccgccgaggcccagatgcgccaggaaattgagaggctgactgagcgactagaagaaaaagagagggagatgcagcagctgctgagccagccccaacacgagcgagagaaggaagtcgtcctgctacggaggagcatggcagaaggggagcgcgcccgggccgccagtgacgtcctgtgccgctccttggccaacgagacccatcagctgcggaggacgctgaccgccaccgcccacatgtgtcagcatctggccaagtgtctggatgaacgacagcatgcacaaaggaatgtgggggagagaagtcctgaccagtcggaacacacagatgggcacacctctgtccagagtgttattgagaagttgcaggaagaaaatcgactgttaaaacagaaggtgactcacgttgaagacctcaatgccaagtggcagcgctacaacgccagcagggacgaatacgtgagggggctccatgcgcagctcagggggctgcagatcccccacgagcccgagctgatgaggaaggagatctcccggctcaacagacagttggaagagaaaataaatgactgtgccgaagtgaagcaggagctggcggcctccaggacggcccgggatgctgcgttggagcgggtgcagatgctggaacagcagattctcgcttacaaggatgacttcatgtcagaaagggccgatcgggaacgggctcaaagtaggattcaagaactggaggaaaaggtcgcctctttgctgcaccaggtgtcctggagacaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 29 - DNA, 2289 bp, synthetic construct
  source 1..2289: mol_type = other DNA
atggatcatttgaacgaggcaactcaggggaaagaacattcagaaatgtctaacaatgtgagtgatccgaagggtccaccagccaagattgcccgcctggagcagaacgggagcccgctaggaagaggaaggcttgggagtacaggtgcaaaaatgcagggagtgcctttaaaacactcgggccatctgatgaaaaccaaccttaggaaaggaaccatgctgccagttttctgtgtggtggaacattatgaaaacgccattgaatatgattgcaaggaggagcatgcagaatttgtgctggtgagaaaggatatgcttttcaaccagctgatcgaaatggcattgctgtctctaggttattcacatagctctgctgcccaggccaaagggctaatccaggttggaaagtggaatccagttccactgtcttacgtgacagatgcccctgatgctacagtagcagatatgcttcaagatgtgtatcatgtggtcacattgaaaattcagttacacagttgccccaaactagaagacttgcctcccgaacaatggtcgcacaccacagtgaggaatgctctgaaggacttactgaaagatatgaatcagagttcattggccaaggagtgccccctttcacagagtatgatttcttccattgtgaacagtacttactatgcaaatgtctcagcagcaaaatgtcaagaatttggaaggtggtacaaacatttcaagaagacaaaagatatgatggttgaaatggatagtctttctgagctatcccagcaaggcgccaatcatgtcaattttggccagcaaccagttccagggaacacagccgagcagcctccatcccctgcgcagctctcccatggcagccagccctctgtccggacacctcttccaaacctgcaccctgggctcgtatcaacacctatcagtcctcaattggtcaaccagcagctggtgatggctcagctgctgaaccagcagtatgcagtgaatagacttttagcccagcagtccttaaaccaacaatacttgaaccaccctccccctgtcagtagatctatgaataagcctttggagcaacaggtttcgaccaacacagaggtgtcttccgaaatctaccagtgggtacgcgatgaactgaaacgagcaggaatctcccaggcggtatttgcacgtgtggcttttaacagaactcaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 30 - DNA, 3513 bp, synthetic construct
  source 1..3513: mol_type = other DNA
atggcgaaggcgacgtccggtgccgcggggctgcgtctgctgttgctgctgctgctgccgctgctaggcaaagtggcattgggcctctacttctcgagggatgcttactgggagaagctgtatgtggaccaggcagccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctgtgttttccagggtgcctgacgattcagattcgcactgtcctgtgtattttcatagtggtcttaatctactcagtagcccaaggttgtgtggtgggctgcctgccttgggcctggagctccaagcccaacagttccctggtggtcctttcgtctgggggccagcgcacagccctgcccaccctgccctgcgagtctacacaccatgccctgctctgctgcctggtgggcaccctcccgctagccatatttttccgggtgtcctccttgccaaaaatgatcctgctctccgggctcaccacgtcctacatcctcgttctggagctcagcggatacaccaggactgggggtggtgccgtctccgggcgcagctacgagccgattgtggccatcctgctcttctcctgtgcgctggccctgcatgccaggcaggtggacatcaggctgaggctggactacctctgggccgcacaggcagaggaggagcgagaggacatggagaaggtgaagctggacaacaggcgcatcctcttcaacctcctgccggcccacgtcgcccagcacttcctcatgtccaaccctcggaacatggacctctactaccagtcctactcccaggtgggcgtcatgtttgcctccatccccaacttcaatgacttctacatcgagctggacggcaacaacatgggggtggagtgtctgcggcttctcaacgagatcatcgccgactttgacgagctcatggaaaaagacttttacaaggacatagagaagatcaagaccatcgggagcacctacatggccgctgtggggctagcgcccacctcggggaccaaggctaagaagtccatctcctcccacctgagcacgctggcggactttgccattgagatgtttgacgttctggatgaaatcaactaccagtcttacaacgactttgtcctccgagttggcatcaatgttggccctgtggtggctggagtgattggcgctcgcaggccccagtacgacatctggggaaacacagtcaacgtggccagtcggatggatagcacaggggtccagggcagaatccaggtgactgaggaagtccaccggctgctgagaaggtgcccctaccactttgtgtgccgaggcaaagtcagtgtcaagggcaaaggcgagatgttgacatactttctagaaggcaggactgatggaaacggctcccaaatcaggtccctgggcttggatcggaaaatgtgtccatttgggagagctggccttcagggcagacgtccccccgtgtgccccatgcctggcgtctcagtcagggctgggctccctccacactccccaggccagtacctgccctctgcagcagctgggaaggaggcttag

SEQ ID NO: 31 - DNA, 3861 bp, synthetic construct
  source 1..3861: mol_type = other DNA
atggcgaaggcgacgtccggtgccgcggggctgcgtctgctgttgctgctgctgctgccgctgctaggcaaagtggcattgggcctctacttctcgagggatgcttactgggagaagctgtatgtggaccaggcagccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaacaagtgtcattcaaggatgtatgtgtggacttcactcaggaagagtggtatctgctggaccctgctcagaagattctatacagagatgtgatcctggaaaattatagcaatcttgtctcagtagggtattgcattactaaaccagaagtgatctttaagatcgagcaaggagaagagccctggatattagaaaaaggattcccaagccagtgccacccagaaaggaaatggaaagttgatgacgtgttagagagcagccaggaaaatgaagatgaccatttttgggagcttctattccacaacaacaaaacagtaagtgtagaaaatggagatagaggaagcaaaactttcaatttgggcacagaccctgtttctttaagaaattatccctataaaatatgtgactcatgtgaaatgaatttgaaaaatatttcgggcttaattattagtaaaaagaactgttccagaaagaagcctgatgagtttaatgtatgtgagaaattgctccttgatattaggcatgagaaaatccctattggagagaagtcttataaatatgatcaaaaaaggaatgccattaattatcaccaggatctcagtcagccaagttttggccaatcttttgagtatagtaaaaatggacaaggcttccatgatgaggcagcattttttacaaataagagatctcagataggagagacagtctgtaaatataacgaatgtggaagaaccttcattgaaagtttaaagctgaatatatctcaaagacctcatttggaaatggagccgtatggatgcagtatttgcgggaagtccttctgcatgaatttaaggtttggacatcagagagctcttacaaaggacaatccttatgaatataatgaatatggggaaatcttctgtgacaattcagctttcattatccatcagggagcttacacaagaaagattctccgtgaatataaagtgagtgacaaaacctgggaaaagtcagctctcttaaaacatcaaatagtacacatggggggaaagtcttatgattacaatgaaaatgggagtaatttcagcaagaagtcacatcttacccagcttcggagagctcacacaggagaaaaaacctttgaatgtggtgaatgtgggaaaaccttctgggagaagtcaaacctcactcaacatcagagaacacacacaggagagaagccctatgaatgtactgaatgtgggaaagccttttgccagaaaccacacctgaccaaccatcagcgaacacatacaggagaaaaaccctatgaatgtaagcaatgtggaaaaacattctgtgtgaagtcaaacctcactgaacatcagagaacacacacaggggagaagccctatgaatgtaatgcatgtgggaaatccttctgccacagatcagccctcactgtgcatcagagaacacacacaggggagaaaccgtttatatgtaatgaatgtggaaaatccttctgtgtgaagtcaaacctcattgtacatcaaagaactcacactggggagaaaccatataagtgtaatgaatgtgggaaaaccttctgtgaaaaatcagctctcactaaacatcagaggactcacacaggggagaagccgtatgagtgtaatgcatgtgggaagacctttagtcagaggtcagtgctcaccaaacatcagagaattcacacaagggtgaaagctctttcaacatcctga

SEQ ID NO: 32 - DNA, 3639 bp, synthetic construct
  source 1..3639: mol_type = other DNA
atggcgaaggcgacgtccggtgccgcggggctgcgtctgctgttgctgctgctgctgccgctgctaggcaaagtggcattgggcctctacttctcgagggatgcttactgggagaagctgtatgtggaccaggcagccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaaggtaacctgggccgggtggaccaggtctctgagtttgagtatgatctgttcattaggccggacacctgtaatccacgcttccgagtctggttcaactttactgttgaaaatgtgaaagaatcacagagggtcattttcaacattgttaacttcagtaaaaccaagagtctctatagagatgggatggcccctatggtgaaatctaccagcagaccaaaatggcaaaggctgccacccaaaaatgtttactactaccgctgcccggaccataggaagaactatgtgatgtcctttgccttttgttttgaccgagaagaagatatttaccagtttgcttactgctacccatatacatacactcgcttccaacattaccttgacagcctgcaaaagagaaacatggattacttctttcgggagcagctgggccagagtgtgcaacaacgaaagcttgacctcctgacgataaccagccctgacaatctccgggaaggggcagagcagaaggtggtattcatcacaggacgagtccacccaggggaaacaccctcatcatttgtgtgccaagggatcattgacttccttgtaagccagcaccctattgcctgtgtcctccgggaatacctggtcttcaagatcgcaccaatgctcaatcctgatggagtctacctgggcaattacaggtgttctctgatgggatttgatctgaatcgtcactggctggatccctctccatgggtccatcctaccctgcatggagtgaaacaactcatcgtccagatgtacaacgacccaaaaacaagcctggagttttatattgacatccatgcccactccaccatgatgaatggcttcatgtatggcaacatctttgaggatgaggaacggttccagaggcaggccatttttcccaagctcctctgccagaatgctgaggacttctcctattccagcacatcctttaaccgggacgctgtgaaagcaggaactggccgtcgcttcctcggtggactcctggaccacacttcctattgctacaccctagaggtctccttctacagctacatcatcagtggcaccacggctgctgtgccctacactgaagaagcctatatgaagctggggcggaatgtggcaagaacctttttggactattatcggctgaaccccgtggttgaaaaggtggcaattcccatgccgagactgcgaaataaagaaatagaagtccagagaaggaaagaaaaatcccctccttacaagcacccactcctgcggggcccagccagcaactaccccaacagcaaaggggacaagaagagctcagtgaaccacaaagacccttcaacccctttttaa

SEQ ID NO: 33 - DNA, 1959 bp, synthetic construct
  source 1..1959: mol_type = other DNA
atggcgaaggcgacgtccggtgccgcggggctgcgtctgctgttgctgctgctgctgccgctgctaggcaaagtggcattgggcctctacttctcgagggatgcttactgggagaagctgtatgtggaccaggcagccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccagggtgtcctggggaagtgtcgctacgtttactatgggaaaaactcagagggcaacaggtttatccgagatgaccagctctga

SEQ ID NO: 34 - DNA, 2694 bp, synthetic construct
  source 1..2694: mol_type = other DNA
atggcgaaggcgacgtccggtgccgcggggctgcgtctgctgttgctgctgctgctgccgctgctaggcaaagtggcattgggcctctacttctcgagggatgcttactgggagaagctgtatgtggaccaggcagccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggctcctgcttacattcgggagtgtgcgagattgcactatttgggatccagaactcaggcatcaaaggatgaccttgacctcatagcttcatgtcatactaagtcctttcagctggcaaagtctcagaaacggaaccgggtagattcctgccctcaccaggaggagacccagcaccatacggaagaggcactgagagagctgtttcaacacgttcatgatatgccagagtcagcaaagaagaaacaacttattagacagtttaataagcaatcattgacccagacaccagggcgagaaccttctacttcccaggtacaaaagagggctcgttcgcgctccttcagtgggcttattaagcggaaggtcctgggaaatcagatgatgtcagaaaagaaaaagaagaaccctactccagaatctgtggccattggtgaattgaagggaaccagcaaagaaaataggaacttattattttctggctctccagctgtcacgatgacaccaacaagattgaagtggtctgaagggaagaaagaggggaaaaaaggatttctctga

SEQ ID NO: 35 - DNA, 2661 bp, synthetic construct
  source 1..2661: mol_type = other DNA
atggcgaaggcgacgtccggtgccgcggggctgcgtctgctgttgctgctgctgctgccgctgctaggcaaagtggcattgggcctctacttctcgagggatgcttactgggagaagctgtatgtggaccaggcagccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggtgcaaattcgaccatggaacctaagtgacagtgactttgtaatggatggttctcagcctttggaccccagaaaaactatctttgttgggggagttccacgaccccttcgagctgttgaactggcaatgatcatggaccgtttgtacggtggtgtctgctatgctggcattgatacggacccagagctgaagtaccccaaaggtgctggccgcgtggcattctccaatcagcagagttacattgcagccatcagcgctcgttttgtgcagcttcagcacaatgacattgacaaacgggttgaagtaaagccatatgtgctggatgatcagatgtgtgatgagtgccagggcacacgctgtggtgggaagtttgccccgttcttctgtgccaacgtcacctgtctgcagtattactgtgaatactgctgggcgagcatacattcccgagccgggcgggagttccacaaaccgctggtgaaggagggaggcgaccgccctcgtcacgtcccgttccgctggagctga

SEQ ID NO: 36 - DNA, 4842 bp, synthetic construct
  source 1..4842: mol_type = other DNA
atggcgaaggcgacgtccggtgccgcggggctgcgtctgctgttgctgctgctgctgccgctgctaggcaaagtggcattgggcctctacttctcgagggatgcttactgggagaagctgtatgtggaccaggcagccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccctatcccaagctccagtgtcctcccttcggctcccagagatgtggtccctgtcttggtttccagccgatttgtccgtctcagctggcgcccacctgcagaagcgaaagggaacattcaaactttcacggtctttttctccagagaaggtgacaacagggaacgagcattgaatacaacacagcctgggtcccttcagctcactgtgggaaacctgaagccagaagccatgtacacctttcgagttgtggcttacaatgaatggggaccgggagagagttctcaacccatcaaggtggccacacagcctgagttgcaagttccagggccagtagaaaacctgcaagctgtatctacctcacctacctcaattcttattacctgggaaccccctgcctatgcaaacggtccagtccaaggttacagattgttctgcactgaggtgtccacaggaaaagaacagaatatagaggttgatggactatcttataaactggaaggcctgaaaaaattcaccgaatatagtcttcgattcttagcttataatcgctatggtccgggcgtctctactgatgatataacagtggttacactttctgacgtgccaagtgccccgcctcagaacgtctccctggaagtggtcaattcaagaagtatcaaagttagctggctgcctcctccatcaggaacacaaaatggatttattaccggctataaaattcgacacagaaagacgacccgcaggggtgagatggaaacactggagccaaacaacctctggtacctattcacaggactggagaaaggaagtcagtacagtttccaggtgtcagccatgacagtcaatggtactggaccaccttccaactggtatactgcagagactccagagaatgatctagatgaatctcaagttcctgatcaaccaagctctcttcatgtgaggccccagactaactgcatcatcatgagttggactcctcccttgaacccaaacatcgtggtgcgaggttatattatcggttatggcgttgggagcccttacgctgagacagtgcgtgtggacagcaagcagcgatattattccattgagaggttagagtcaagttcccattatgtaatctccctaaaagcttttaacaatgccggagaaggagttcctctttatgaaagtgccaccaccaggtctataaccgatcccactgacccagttgattattatcctttgcttgatgatttccccacctcggtcccagatctctccacccccatgctcccaccagtaggtgtacaggctgtggctcttacccatgatgctgtgagggtcagctgggcagacaactctgtccctaagaaccaaaagacgtctgaggtgcgactttacaccgtccggtggagaaccagcttttctgcaagtgcaaaatacaagtcagaagacacaacatctctaagttacacagcaacaggcctcaaaccaaacacaatgtatgaattctcggtcatggtaacaaaaaacagaaggtccagtacttggagcatgactgcacatgccaccacgtatgaagcagcccccacctctgctcccaaggacttgacagtcattactagggaagggaagcctcgtgccgtcattgtgagttggcagcctcccttggaagccaatgggaaaattactgcttacatcttattttataccttggacaagaacatcccaattgatgactggattatggaaacaatcagtggtgataggcttactcatcaaatcatggatctcaaccttgatactatgtattactttcgaattcaagcacgaaattcaaaaggagtggggccactctctgatcctatcctcttcaggactctgaaagtggaacaccctgacaaaatggctaatgaccaaggtcgtcatggagatggaggttattggccagttgatactaatttgattgatagaagcaccctaaatgagccgccaattggacaaatgcaccccccgcatggcagtgtcactcctcagaagaacagcaacctgcttgtgatcattgtggtcaccgttggtgtcatcacagtgctggtagtggtcatcgtggctgtgatttgcacccgacgctcttcagcccagcagagaaagaaacgggccacccacagtgctggcaaaaggaagggcagccagaaggacctccgaccccctgatctttggatccatcatgaagaaatggagatgaaaaatattgaaaagccatctggcactgaccctgcaggaagggactctcccatccaaagttgccaagacctcacaccagtcagccacagccagtcagaaacccaactgggaagcaaaagcacctctcattcaggtcaagacactgaggaagcagggagctctatgtccactctggagaggtcgctggctgcacgccgagccccccgggccaagctcatgattcccatggatgcccagtccaacaatcctgctgtcgtgagcgccatcccggtgccaacgctagaaagtgcccagtacccaggaatcctcccgtctcccacctgtggatatccccacccgcagttcactctccggcctgtgccattcccaacactctcagtggaccgaggtttcggagcaggaagaagtcagtcagtgagtgaaggaccaactacccaacaaccacctatgctgcccccatctcagcctgagcattctagcagcgaggaggcaccaagcagaaccatccccacagcttgtgttcgaccaactcacccactccgcagctttgctaatcctttgctacctccaccaatgagtgcaatagaaccgaaagtcccttacacaccacttttgtctcagccagggcccactcttcctaagacccatgtgaaaacagcctcccttgggttggctggaaaagcaagatcccctttgcttcctgtgtctgtgccaacagcccctgaagtgtctgaggagagccacaaaccaacagaggattcagccaatgtgtatgaacaggatgatctgagtgaacaaatggcaagtttggaaggactcatgaagcagcttaatgccatcacaggctcagccttttaa

SEQ ID NO: 37 - DNA, 3234 bp, synthetic construct
  source 1..3234: mol_type = other DNA
atggcgaaggcgacgtccggtgccgcggggctgcgtctgctgttgctgctgctgctgccgctgctaggcaaagtggcattgggcctctacttctcgagggatgcttactgggagaagctgtatgtggaccaggcagccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctgaatcatgtcaaccctgccatggacttcacgcagactccacctgggatgttggctctggacaacatgctgtactttgccaagcaccaccaagatgcctacatccggattgtgcttgagaacagtagtcgagaagacaagcatgaatgtccctttggccgcagtagtatagagctgaccaagatgctatgtgagatcttgaaagtgggcgagttgcctagtgagacctgcaacgacttccacccgatgttcttcacccacgacagatcctttgaggagtttttctgcatctgtatccagctcctgaacaagacatggaaggaaatgagggcaacttctgaagacttcaacaaggtaatgcaggtggtgaaggagcaggttatgagagcacttacaaccaagcctagctccctggaccagttcaagagcaaactgcagaacctgagctacactgagatcctgaaaatccgccagtccgagaggatgaaccaggaagatttccagtcccgcccgattttggaactaaaggagaagattcagccagaaatcttagagctgatcaaacagcaacgcctgaaccgccttgtggaagggacctgctttaggaaactcaatgcccggcggaggcaagacaagttttggtattgtcggctttcgccaaatcacaaagtcctgcattacggagacttagaagagagtcctcagggagaagtgccccacgattccttgcaggacaaactgccggtggcagatatcaaagccgtggtgacgggaaaggactgccctcatatgaaagagaaaggtgcccttaaacaaaacaaggaggtgcttgaactcgctttctccatcttgtatgactcaaactgccaactgaacttcatcgctcctgacaagcatgagtactgtatctggacggatggactgaatgcgctactcgggaaggacatgatgagcgacctgacgcggaatgacctggacaccctgctcagcatggaaatcaagctccgcctcctggacctggaaaacatccagatccctgacgcacctccgccgattcccaaggagcccagcaactatgacttcgtctatgactgtaactga

SEQ ID NO: 38 - DNA, 5622 bp, synthetic construct
  source 1..5622: mol_type = other DNA
atggcgaaggcgacgtccggtgccgcggggctgcgtctgctgttgctgctgctgctgccgctgctaggcaaagtggcattgggcctctacttctcgagggatgcttactgggagaagctgtatgtggaccaggcagccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccaggattttgtgtcatgtatagagaactacagaagaagaggacaagagctatatgcatctttatacaaagaccatgtgcaaaggcgaaaatgtggcaacatcaaggcagccaacgcctgggccaggatccaggagcagctttttggggagctgggcttgtggagccagggggaagaaaccaagccctgttccccatgggaactcgactggagagaaggaccagctcgaatgaggaaacgcatcaaacgcttgtctcctttggaggccctgagctcaggaaggcacaaggaaagccaagacaaaaatgatcatatttctcaaacaaatgctgaaaaccaagatgaactgacactgagggaggctgagggcgagccggacgaggtgggggtggactgcacccagctgaccttcttcccagccttacacgaaagtctgcactcagaagacttcttggaactgtgtcgggaaagacaagttattttacaagagcttcttgataaagaaaaggtgacgcagaagttctccctggtgattgtgcagggccacctggtgtcagaaggggtcctgctttttggccaccaacacttctacatctgcgagaacttcacactgtctcccacgggtgatgtctactgtacccgtcactgcttatccaacatcagcgatccgttcattttcaacctgtgcagcaaagacaggtccactgaccattactcgtgccagtgccacagctacgctgacatgcgggagctacggcaggctcgcttcctcctgcaggacatcgccctggagatcttcttccacaatggatattccaagtttcttgtcttctacaacaatgatcggagtaaggcctttaaaagcttctgctctttccaacccagcctgaaggggaaagccacctcggaggacaccctcagtctaaggagataccccggctctgacaggatcatgctgcagaagtggcagaaaagggacatcagcaattttgagtatctcatgtacctcaacaccgcggctgggagaacctgcaatgactacatgcagtacccagtgttcccctgggtcctcgcagactacacctcagagacattgaacttggcaaatccgaagattttccgggatctttcaaagcccatgggggctcagaccaaggaaaggaagctgaaatttatccagaggtttaaagaagttgagaaaactgaaggagacatgactgtccagtgccactactacacccactactcctcggccatcatcgtggcctcctacctggtccggatgccacccttcacccaggccttctgcgctctgcagggcggaagcttcgacgtggcagacagaatgttccacagtgtgaagagcacgtgggagtcggcctccagagagaacatgagtgacgtcagggagctgaccccagagttcttctacctgcctgagttcttaaccaactgcaacggggtagagttcggctgcatgcaggacgggactgtgctaggagacgtgcagctccctccctgggctgatggggaccctcggaaattcatcagcctgcacagaaaggccctggaaagtgactttgtcagtgccaacctccaccattggatagaccttatttttgggtacaagcagcaggggccagccgcagtggatgctgttaatatcttccacccctacttctacggtgacagaatggacctcagcagcatcactgaccccctcatcaaaagcaccatcctggggtttgtcagcaactttggacaggtgcccaaacagctctttaccaaacctcacccagccaggactgcagcagggaagcctctgcctggaaaggatgtctccacccccgtgagcctgcctggccacccacagccctttttctacagcctgcagtcgctgaggccctcccaggtcacggtcaaagatatgtacctcttttctctaggctcagagtcccccaaaggggccattggccacattgtctctactgagaagaccattctggctgtagagaggaacaaagtgctgctgcctcctctctggaacaggaccttcagctggggctttgatgacttcagctgctgcttggggagctacggctccgacaaggtcctgatgacattcgagaacctggctgcctggggccgctgtctgtgcgccgtgtgcccatccccaacaacgattgtcacctctgggaccagcactgtggtgtgtgtgtgggagctcagcatgaccaaaggccgcccgaggggcttgcgcctccggcaggccttgtatggacacacacaggctgtcacgtgcctggcagcgtcagtcaccttcagcctcctggtgagcggctcccaggactgcacctgtatcctgtgggatctggaccacctcacccacgtgacccgcctgcccgcccatcgggaaggcatctcagccatcaccatcagtgacgtctcaggcaccattgtctcctgtgcgggagcacacttgtccctgtggaatgtcaatggacagcccctggccagcatcaccacagcctggggcccagaaggagccataacctgctgctgcctgatggagggcccagcatgggacacaagccagatcatcatcaccgggagtcaagacggcatggtccgggtttggaagactgaggatgtgaagatgtctgttcctggacggccagcaggagaggagcccccggctcagcctccaagcccaagaggccacaagtgggagaagaacctggccttgagtcgagagctggacgttagcattgctttgacagggaagcccagcaaaaccagccccgcagtgactgctctggccgtgtccagaaaccacaccaaactcctggttggtgatgagagggggagaatattctgctggtctgcagatgggtag

SEQ ID NO: 39 - DNA, 5298 bp, synthetic construct
  source 1..5298: mol_type = other DNA
atgaagaagatttttagtaagaagggcgagtcgcccttgggctccttcgcgcggcggcagaggagcagcgcgggaggcgggggcgagccgggggagggcgcctactcgcagcccggctaccacgtccgagaccgagatctcggcaagatccacaaagctgccagcgcgggtaatgtggcgaaagtgcagcagatccttttgctcaggaagaatggcttgaacgatagagacaagatgaacaggacggctctacatttggcctgtgccaatggtcatccagaagtagtaactctcctggtggacagaaaatgccagctcaatgtctgtgacaacgaaaacaggacagctctgatgaaggctgtacaatgccaggaagagaaatgtgcaactattctgctagaacatggtgctgatccaaatcttgcggatgtccatggcaacactgctcttcactatgctgtctataatgaggacatatcagtagcaacaaagctgcttttgtatgatgcaaatattgaagcaaaaaacaaggatgacctcacaccacttttacttgcagtaagtggaaaaaagcagcaaatggtggaatttttaataaagaaaaaagcaaatgtaaatgcagtagataagttggaaagcagtcaccaactaatttcagaatataaagaagaaaggatacctaaacattcttctcaaaatagtaattcagtggatgaaagctctgaagactccttaagcaggctttctggcaaaccgggtgttgatgattcatggcctacctcagatgacgaagacctcaattttgatactaagaatgtcccaaaaccaagcttagcaaagctaatgactgcttctcagcaatccaggaaaaatttagaagcaacatatggcactgtgagaacaggaaatagaactttgtttgaggatagagattccgatagtcaagatgaagttgtggttgaaagccttcctacaacatcaatcaaagtccagtgcttttctcatcctacctatcaatcacctgaccttcttccaaaaccttcccacaagtcgttagcaaaccctggtcttatgaaggaagaaccaacaaagccaggcattgcaaaaaaagaaaatggtattgatattattgaaagtgctccactagagcaaacaaataatgacaatttgacttatgttgatgaagtgcacaaaaataatagaagtgatatgatgtccgcattaggattaggacaagaggaagatatagaatcaccttgggattctgagagtatctctgagaattttccacagaagtatgttgatcctttagctggggctgcagacggaaaagaaaaaaatataggaaatgaacaagcagaagatgtgttttatataccttcttgcatgagtggatcaagaaactttaagatggctaaactagaggatacaagaaatgtaggcatgccagtagcccacatggagtctcctgagagatatcttcacttgaagcctaccattgaaatgaaagattctgttccaaataaagcaggaggaatgaaggatgtacaaacatccaaagcagctgaacatgacttagaagtagcatcagaagaagagcaagaaagggaagggagtgaaaataaccagccacaggttgaagaagaaaggaaaaaacacagaaataatgaaatggaagtatcagcaaacatacatgatggtgctactgatgatgctgaagatgatgatgatgatgatggattaattcaaaaaagaaagagtggagaaactgatcatcagcaatttcccaggaaggaaaataaagagtatgctagtagtggtcctgccttgcaaatgaaggaagtaaagagcactgaaaaagaaaaacggacctcgaaagaatctgtgaattcaccagtgtttgggaaggccagtttactaactggtggcctgctacaagtggatgatgacagcagtttaagtgaaatagatgaggatgaaggaaggcctactaagaaaacatctaatgaaaagaacaaggtcaaaaaccaaatacagtctatggatgatgttgatgacttaactcagtcatctgaaacagcctcagaggattgtgagctaccccactctagttacaagaattttatgttgctcattgaacaacttggaatggagtgtaaagattctgttagcctattgaaaatccaggatgcagctctttcatgtgaaagattattagaacttaaaaaaaatcactgtgaactacttacagtaaaaattaaaaaaatggaagacaaggttaatgtactacaaagggagctatctgaaacaaaagaaataaaatcacagttagagcatcaaaaagttgaatgggaacgagaactgtgctctttgagatttagcttaaaccaagaagaagagaagagaagaaatgctgatacgttgtatgaaaaaattagggaacagttaagaagaaaagaagagcaatataggaaagaagttgaagtgaaacaacagcttgaactgagtctccaaacactggagatggaattgaggactgtaaaaagtaatttgaatcaggtcgttcaagagcgaaatgacgctcagaggcaactttctcgagaacagaatgccagaatgttacaagatggaattctgaccaatcacctttccaaacaaaaggagattgaaatggctcaaaagaaaatgaattctgagaattctcatagtcatgaagaagaaaaagacctatcgcataaaaatagcatgttgcaggaagaaattgctatgctaagactagaaatagacacaataaaaaatcaaaaccaggaaaaagaaaagaaatgttttgaggaccttaaaattgtaaaagaaaagaatgaagaccttcagaagactataaaacagaatgaggaaacattaacacaaacaatatcccagtataatggacggcttagtgttctgacagctgagaatgcaatgctaaattctaaactggagaatgaaaagcaaagcaaggaaagactggaagcagaagttgaatcataccattctagattggctgctgctatacatgatcgtgatcaaagtgagacatcaaaaagagaactagaacttgctttccagagagcaagagatgaatgttctcgtttacaggacaaaatgaattttgatgtgtctaacctaaaagataacaatgagattctttctcaacaactatttaaaactgaaagtaaactcaatagcctagaaattgagttccatcacacgagagatgccctcagagaaaagactttgggtttagaacgggtacaaaaggacctaagccaaacacagtgtcaaatgaaggaaatggaacaaaagtatcaaaatgaacaagttaaagtgaataaatacattggaaagcaggagtctgtagaggagagattgtctcaactacaaagtgagaatatgttgcttcgacaacaactggatgatgcccacaacaaggctgacaataaagagaagacagtgattaatatccaagaccagtttcatgctattgtgcaaaaacttcaagctgagagtgaaaagcaaagtcttctgctagaagaaagaaataaggagttaatcagtgaatgtaatcacttaaaagaaagacagtatcaatatgaaaatgaaaaggcagaaagagaagttgttgtgagacaacttcaacaagaactagctgataccctaaaaaaacaatctatgtcagaggcttcactggaggttacgtcacgttatcgtattaatttagaagatgagacacaggatttaaagaagaaattaggtcaaatcagaaatcaattgcaagaagcacaggatcgacatacagaagctgtcagatgtgctgagaagatgcaagatcacaagcaaaagcttgaaaaagataatgccaagttaaaagttacagtcaaaaagcaaatggacaaaattgaggagcttcagaaaaacctgttaaatgcaaatttgtctgaagatgaaaaggaacaattaaagaaacttatggaattaaaacagtcactggaatgtaatttggatcaagaaatgaagaaaaatgttgaattagaaagagagataactggatttaagaacctcttaaaaatgacaagaaagaagttaaatgaatatgaaaatggagaatttagtttccatggagatttaaaaactagtcaatttgaaatggatattcagattaataagctaaaacataaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 40 - DNA, 4299 bp, synthetic construct
  source 1..4299: mol_type = other DNA
atgagtatgctaaagccaagtgggcttaaggcccccaccaagatcctgaagcctggaagcacagctctgaagacacctacggctgttgtagctccagtagaaaaaaccatatccagtgaaaaagcatcaagcactccatcatctgagactcaggaggaatttgtggatgactttcgagttggggagcgagtttgggtgaatggaaataagcctggatttatccagtttcttggagaaacccagtttgcaccaggccagtgggctggaattgttttagatgaacccataggcaagaacgatggttcggtggcaggagttcggtatttccagtgtgaacctttaaagggcatatttacccgaccttcaaagttaacaaggaaggtgcaagcagaagatgaagctaatggcctgcagacaacgcccgcctcccgagctacttcaccgctgtgcacttctacggccagcatggtgtcttcctccccctccaccccttcaaacatccctcagaaaccatcacagccagcagcaaaggaaccttcagctacgcctccgatcagcaaccttacaaaaactgccagtgaatctatctccaacctttcagaggctggctcaatcaagaaaggagaaagagagctcaaaatcggagacagagtattggttggtggcactaaggctggtgtagtccggtttcttggggagaccgactttgccaagggggagtggtgtggcgtggagttagatgagccacttgggaagaatgatggcgctgttgctggaacaaggtattttcagtgtcaacccaaatatggcttgttcgctcctgtccacaaagttaccaagattggcttcccttccactacaccagccaaagccaaggccaacgcagtgaggcgagtgatggcgaccacgtccgccagcctgaagcgcagcccttctgcctcttccctcagctccatgagctcagtggcctcctctgtgagcagcaggcccagtcggacaggactattgactgaaacctcctcccgttacgccaggaagatctccggtaccactgccctccaggaggccctgaaggagaagcagcagcacattgagcagctgctggcggaacgggatctggagagggcggaggtggccaaggccacgagccacgtgggggagatagagcaggagctagctctggcccgggacggacatgaccagcatgtcctggaattggaagccaaaatggaccagctgcgaacaatggtggaagctgctgacagggagaaggtggagcttctcaaccagcttgaagaggagaaaaggaaggttgaggaccttcagttccgggttgaagaagaatcaattaccaaaggtgatcttgagacgcagaccaaactggagcatgcccgcattaaggagcttgaacagagcctgctctttgaaaagaccaaagctgacaaactccagagggagttagaagacactagggtggctacagtttcagaaaagtcacgtataatggaactggagaaagacctagcattgagagtacaggaagtagctgagctccgaagaaggctagagtccaataagcctgctggggatgtggacatgtcactttcccttttgcaagagataagctctttgcaagaaaagttagaagtcacccgtactgaccaccagagagaaataacttctctgaaggagcattttggagcccgggaagaaactcatcagaaggagataaaggctctgtataccgccacggaaaagctttccaaagagaacgagtcattgaaaagcaagctggagcatgccaacaaagagaactcagatgtgatagctctatggaagtccaaactggagactgccatcgcatcccaccagcaggcgatggaagaactgaaggtatctttcagcaaagggcttggaacagagacggcagaatttgctgaactaaaaacacaaatagagaaaatgagactagattaccaacacgaaatagaaaatttgcagaatcaacaagactctgaacgggctgcccatgctaaagagatggaagccttgagggctaaactgatgaaagttattaaagaaaaggaaaacagtctggaagccatcaggtcgaaactggacaaagcagaagaccagcatctcgtagaaatggaagacacgttaaacaaattacaggaagctgaaataaaggtaaaggagctagaggtactgcaagccaaatgcaatgaacaaaccaaggttattgataattttacatcacagctcaaggctactgaagaaaagctcttggatcttgatgcacttcggaaagccagttccgaaggtaaatcggaaatgaagaaacttagacagcagcttgaggcagctgagaaacagattaaacatttagagattgaaaagaatgctgaaagtagcaaggctagtagcattaccagagagctccaggggagagagctaaagcttactaaccttcaggaaaatttgagtgaagtcagtcaagtgaaagagactttggaaaaagaacttcagattttgaaagaaaagtttgctgaagcttcagaggaggcagtctctgttcagagaagtatgcaagaaactgtaaataagttacaccaaaaggaggaacagtttaacatgctgtcttctgacttggagaagctgagagaaaacttagcagatatggaggcaaaatttagagagaaagatgagagagaagagcagctgataaaggcaaaggaaaaactggaaaatgacattgcagaaataatgaagatgtcaggagataactcttctcagctgacaaaaatgaacgatgaattacgtctgaaagaaagagatgtagaagaattacagctaaaacttacaaaggctaatgaaaatgcaagttttctgcaaaaaagtattgaggacatgactgtcaaagctgaacagagccagcaagaagcagctaaaaagcatgaggaagaaaagaaagaattggagaggaaattgtcggacctggaaaagaaaatggaaacaagccacaaccagtgtcaggagctgaaagccaggtatgagagagccacttctgagacaaaaaccaagcatgaagaaatcctacagaacctccagaagacgctgctggacacagaggacaagctgaagggcgcacgggaggagaacagtggcttgctgcaggagctggaggagctgagaaagcaagccgacaaagccaaagaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 41 - DNA, 3465 bp, synthetic construct
  source 1..3465: mol_type = other DNA
atggagccccagcgccgggagctgctcgcccagtgtcagcagagcctggcccaggccatgacggaggtggaagccgtgctcgggctgctcgaggccgcgggagcgctcagtcccggcgagcggcggcagctggacgaggaggcgggaggcgccaaggcggagctgctgctcaagctgctcttggccaaggagcgggaccacttccaggacctgcgggcggcgctggagaagacgcagcctcacctgctgcccattctctacctgaacggcgtcgtcgggccgccgcagcccgccgaaggcgcgggttctacctacagcgtcctgtccaccatgccctcagactcagaaagcagcagctccctcagcagtgtgggcactaccgggaaggcgccgtccccaccacccctcctcactgaccagcaagtgaatgagaaggtggagaacctctccattcagctgcggctgatgacccgggagagaaacgagctccgcaagcgcctggcctttgctacgcatggcacggcctttgacaagaggccctaccacaggctgaatcctgactatgagaggctgaagatccagtgcgtgcgagccatgtcggacctgcagagcctgcagaaccagcacaccaacgccttgaagaggtgtgaggaggtggccaaggagactgacttctaccacacactccacagccggctcctgagtgaccagactcggctgaaggatgacgtggacatgctgaggcgggagaatgggcagctgctgcgggagcgaaacctgctgcagcagtcatgggaggacatgaagcggctccacgaggaggaccagaaggagatcggtgacctccgtgcccagcagcagcaggtgttgaagcacaacgggtcatccgagattctcaacaaactgtatgacacggccatggacaagttggaggtggtcaagaaggactatgacgcccttcggaagaggtacagtgagaaagtcgccatccacaatgcagacctgagccgcctggagcagctgggggaggagaaccagcggttgctgaagcagacagagatgctgacccagcagagggacacggccatccagctgcagcaccagtgcgccctctccctgaggaggtttgaggcgatccaccatgagctgaacaaggccacggcgcagaacaaggacctgcagtgggagatggagctgctgcagtcagagctgaccgagctgagaaccacgcaggtgaagacagcaaaggagtcggagaaatacagggaggagcgggacgctgtgtacagcgagtacaagctcatcatgagtgagcgtgaccaggtcatctctgagctggacaagctgcagaccgaagtggagctggccgagtccaagctcaagagcagcacatctgagaagaaggcggccaatgaggagatggaggcgctgcggcagatcaaagacacggtgacaatggatgctgggagagccaacaaggaggttgaaatccttcgaaagcagtgcaaggctctgtgccaggagctgaaggaagccctccaggaggcggatgtggccaagtgccggcgggactgggccttccaggagcgagacaagattgtagcagagcgtgacagcatccggacactgtgtgacaacctgaggcgggagcgggaccgtgcggtgagcgagctggctgaggccctgcgcagcctggatgacacccgcaagcagaagaatgatgtcagccgcgagctgaaggagctcaaggaacagatggaatcccagttggaaaaggaggcccggttccgacagctgatggcccacagctcccacgactcggccattgacacggattccatggagtgggaaacggaagttgtagagttcgagagggagacggaggatattgacttgaaggcactggggtttgatatggcagaaggtgtgaatgagccttgtttcccgggggactgtggcatatttgtcactaaagtggacaaaggaagcattgctgatggccgcttaagggtcaatgactggctgctgagaatcaacgatgtggacctcatcaacaaggacaagaagcaggccatcaaggcgctcctcaatggggagggggccatcaacatggtcgtgcggcggaggaagtccctgggtgggaaggtggtcacgccgctgcacatcaacctcagtggacagaaagacagtggcatcagtctggagaatggagtgtatgctgccgctgtgctgcctggaagccctgccgctaaagaagggtcccttgctgtgggagacaggatcgttgcgatcaatggcattgcactggacaacaagtctctgaatgaatgtgaatctctgctgcgcagctgccaggactccctgaccctgtccctcctgaaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 42 - DNA, 6861 bp, synthetic construct
  source 1..6861: mol_type = other DNA
atggagccccagcgccgggagctgctcgcccagtgtcagcagagcctggcccaggccatgacggaggtggaagccgtgctcgggctgctcgaggccgcgggagcgctcagtcccggcgagcggcggcagctggacgaggaggcgggaggcgccaaggcggagctgctgctcaagctgctcttggccaaggagcgggaccacttccaggacctgcgggcggcgctggagaagacgcagcctcacctgctgcccattctctacctgaacggcgtcgtcgggccgccgcagcccgccgaaggcgcgggttctacctacagcgtcctgtccaccatgccctcagactcagaaagcagcagctccctcagcagtgtgggcactaccgggaaggcgccgtccccaccacccctcctcactgaccagcaagtgaatgagaaggtggagaacctctccattcagctgcggctgatgacccgggagagaaacgagctccgcaagcgcctggcctttgctacgcatggcacggcctttgacaagaggccctaccacaggctgaatcctgactatgagaggctgaagatccagtgcgtgcgagccatgtcggacctgcagagcctgcagaaccagcacaccaacgccttgaagaggtgtgaggaggtggccaaggagactgacttctaccacacactccacagccggctcctgagtgaccagactcggctgaaggatgacgtggacatgctgaggcgggagaatgggcagctgctgcgggagcgaaacctgctgcagcagtcatgggaggacatgaagcggctccacgaggaggaccagaaggagatcggtgacctccgtgcccagcagcagcaggtgttgaagcacaacgggtcatccgagattctcaacaaactgtatgacacggccatggacaagttggaggtggtcaagaaggactatgacgcccttcggaagaggtacagtgagaaagtcgccatccacaatgcagacctgagccgcctggagcagctgggggaggagaaccagcggttgctgaagcagacagagatgctgacccagcagagggacacggccatccagctgcagcaccagtgcgccctctccctgaggaggtttgaggcgatccaccatgagctgaacaaggccacggcgcagaacaaggacctgcagtgggagatggagctgctgcagtcagagctgaccgagctgagaaccacgcaggtgaagacagcaaaggagtcggagaaatacagggaggagcgggacgctgtgtacagcgagtacaagctcatcatgagtgagcgtgaccaggtcatctctgagctggacaagctgcagaccgaagtggagctggccgagtccaagctcaagagcagcacatctgagaagaaggcggccaatgaggagatggaggcgctgcggcagatcaaagacacggtgacaatggatgctgggagagccaacaaggaggttgaaatccttcgaaagcagtgcaaggctctgtgccaggagctgaaggaagccctccaggaggcggatgtggccaagtgccggcgggactgggccttccaggagcgagacaagattgtagcagagcgtgacagcatccggacactgtgtgacaacctgaggcgggagcgggaccgtgcggtgagcgagctggctgaggccctgcgcagcctggatgacacccgcaagcagaagaatgatgtcagccgcgagctgaaggagctcaaggaacagatggaatcccagttggaaaaggaggcccggttccgacagctgatggcccacagctcccacgactcggccattgacacggattccatggagtgggaaacggaagttgtagagttcgagagggagacggaggatattgacttgaaggcactggggtttgatatggcagaaggtgtgaatgagccttgtttcccgggggactgtggcatatttgtcactaaagtggacaaaggaagcattgctgatggccgcttaagggtcaatgactggctgctgagaatcaacgatgtggacctcatcaacaaggacaagaagcaggccatcaaggcgctcctcaatggggagggggccatcaacatggtcgtgcggcggaggaagtccctgggtgggaaggtggtcacgccgctgcacatcaacctcagtggacagaaagacagtggcatcagtctggagaatggagtgtatgctgccgctgtgctgcctggaagccctgccgctaaagaagggtcccttgctgtgggagacaggatcgttgcgatcaatggcattgcactggacaacaagtctctgaatgaatgtgaatctctgctgcgcagctgccaggactccctgaccctgtccctcctgaaggtattccctcagagctcctcgtggagtggccagaacatttttgaaaatatcaaagactctgataagatgctgagttttcgagcccatggcccggaggtccaggctcataacaaacggaacttgatacagcacaataactccacgcagacagacatcttctacacggacaggctggaagacaggaaggagccaggccccccaggaggcagcagctcctttctgcataagccattccctgggggacccttgcaggtctgcccccaggcctgtcccagtgcctctgagcgtagcctgagctccttccgctcagatgcctctggggaccgtggctttgggctggtggacgtgcgtggccggcggccactgctgccctttgagaccgaggtgggcccctgtggggttggggaggcctccctggacaaggcagactctgaaggctccaacagcggcgggacctggcccaaggccatgctcagctccacggcagtgcctgagaagctctctgtttataaaaagccaaagcaaagaaagtccatctttgaccctaacactttcaaacgcccccagacaccccccaaaatagactacctgcttccaggtcctgggcctgctcactctccccagccctccaagagggcggggcctctgacacccccaaaacctcccagaaggagcgactccattaagttccagcacaggctggagactagctccgagtcagaagccactctggtgggcagctccccatccactagtcccccgagcgccctgccccctgacgtggaccccggggagcccatgcacgcatcaccccctcgcaaggccagggtccgcattgcttccagctactaccctgaaggagatggggactcctcccacctgccggccaagaaatcctgtgatgaggacctcacctcccagaaggtggatgagctggggcagaagcgtcgccggccaaaatctgctcccagttttcggccgaagcttgctccagtagtgattcctgctcagttcctggaggaacagaagtgtgtcccggccagtggagaactctccccggagctccaggagtgggcaccttactcgcctgggcattccagccggcacagcaaccccccgctataccctagcaggccgtctgtgggcactgttccccggagtttgacccccagcaccactgtgagctccatcctgcggaaccccatctacactgtgcgcagtcacagggtcggcccctgcagctctccacctgcggcccgagatgctggcccccagggtttgcatcccagtgtccagcaccagggacgcctgagcctggacctgagccacaggacctgcagcgactactccgagatgagagccacccatgggtccaactcactgccctccagcgcccgcctgggttcttcgagtaacttgcagttcaaggcggaacgcattaaaatcccatcaacaccaagatatccgcggagtgtcgtgggctccgagagaggttcagtgtcacattctgaatgcagcactcctccacagtcacccctgaacatcgacaccctgtcctcttgtagccagtcccagacctcagcctccacattgcccagaatcgctgtcaaccccgcgtccctcggggagcggagaaaggacaggccttatgtggaggagccacgccacgtgaaggtgcagaagggctcagagccgctgggcatctccatcgtgagtggagagaagggcggcatctacgtctccaaggtgaccgtggggagcatcgctcaccaggctggcctcgagtatggggatcagttactggagttcaacggcataaacctgcggagcgccacggagcagcaggcgcggctcatcatcgggcagcagtgtgataccatcaccatcctggcccagtacaacccccacgtgcaccagctcagcagccactcccggtccagctcacacctggaccctgccggtacccactccactctccagggcagtggcaccaccaccccggagcatccatctgtcatcgacccactgatggagcaggacgaggggcctagcacccccccagccaagcagagcagctccaggattgcgggagatgccaacaagaagaccctggagccacgcgttgtcttcatcaaaaagtcccagctggagcttggggtgcacttgtgtggtgggaacctgcatggggtgtttgtggccgaggtggaggatgacagtcctgccaagggtcctgacggcctcgtgccaggggacctcatcctggagtatggcagcctggacgtgcggaacaagacagtggaggaagtctatgtggagatgctgaagcccagggatggcgtccgcctgaaggtgcagtaccgccctgaggagttcacgaaggccaagggcctgcctggtgacagcttctacatcagggccctgtacgaccggctggcagatgtggagcaagagttgagctttaagaaggacgacatcctctacgtggatgacaccttaccccagggcacgttcgggtcctggatggcttggcagctggacgagaatgcccagaagatccagcgcgggcagattcccagcaaatatgtgatggaccaagaattctccaggaggctcagcatgtctgaagtcaaagatgacaatagcgccacaaagacgctgtcagcggctgcacgccggtccttttttcggaggaaacacaagcacaaacgcagcgggtccaaggacgggaaagacctgctcgccttggatgccttttccagtgactccattccactctttgaagatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 43 - DNA, 3486 bp, synthetic construct
  source 1..3486: mol_type = other DNA
atgtatggaagtgcccgctctgttgggaaggtggagccgagcagccagagccctgggcgttcacccaggcttccacgttcccctcgcttgggtcaccgtcgaaccaacagtacgggagggagttcgggaagcagtgttggaggtggcagtgggaaaaccctttcaatggaaaatatacaatctttaaatgctgcctatgccacctctggccctatgtatctaagtgaccatgaaaatgtgggttcagaaacacctaaaagcaccatgacacttggccgttctgggggacgtctgccttacggtgttcggatgactgctatgggtagtagccccaatatagctagcagtggggttgctagtgacaccatagcatttggagagcatcacctccctcctgtgagtatggcatccactgtacctcactcccttcgtcaggcgagagataacacaatcatggatctgcagacacagctgaaggaagtattaagagaaaatgatctcttgcggaaggatgtggaagtaaaggagagcaaattgagttcttcaatgaatagcatcaagaccttctggagcccagagctgaagaaggaacgagccctgagaaaagatgaagcttccaaaatcaccatttggaaggaacagtacagagttgtacaggaggaaaaccagcacatgcagatgacaatccaggctctccaggatgaattgcggatccagagggacctgaatcagctgtttcagcaggatagtagcagcaggactggcgaaccttgtgtagcagagctgacagaggagaactttcagaggcttcatgctgagcatgagcggcaggccaaagagctgtttcttcttcgaaagacattggaggaaatggagctgcgtattgagactcaaaagcagaccctaaatgctcgggatgaatccattaagaagcttctggaaatgttgcagagcaaaggactttctgccaaggctaccgaggaagaccatgagagaacaagacgactggcagaggcagagatgcacgttcatcacctagaaagccttttggagcagaaggaaaaagagaacagtatgttgagagaggagatgcatcgaaggtttgagaatgctcctgattctgccaaaacaaaagctctgcaaactgttattgagatgaaggattcaaaaatttcctctatggagcgtgggcttcgagacctggaagaggaaattcagatgctgaaatcgaatggtgctttgagtactgaggaaagggaagaagaaatgaagcaaatggaagtgtatcggagccattctaaatttatgaaaaataagattggccaggtgaaacaggagctgtccagaaaggacacagaactactcgccctgcagacaaagctagaaacactcacaaaccagttctcagatagtaaacagcacattgaagtgttgaaggagtccttgactgctaaggagcagagggctgccatcctgcagactgaggtggatgctctccgattgcgtttggaagagaaggaaaccatgttgaataaaaagacaaaacaaattcaggatatggctgaagagaaggggacacaagctggagagatacatgacctcaaggacatgttggatgtgaaggagcggaaggttaatgttcttcagaagaagattgaaaatcttcaagagcagcttagagacaaggaaaagcagatgagcagcttgaaagaacgggtcaaatccttgcaggctgacaccaccaacactgacactgccttgacaactttggaggaggcccttgcagagaaagagcggacaattgaacgcttaaaggagcagagggacagagatgagcgagagaagcaagaggaaattgataactacaaaaaagatcttaaagacttgaaggaaaaagtcagcctgttgcaaggcgacctttcagagaaagaggcttcacttttggatctgaaagagcatgcttcttctctggcatcctcaggactgaaaaaggactcacggcttaagacactagagattgctttggagcagaagaaggaggagtgtctgaaaatggaatcacaattgaaaaaggcacatgaggcagcattggaagccagagccagtccagagatgagtgaccgaatacagcacttggagagagagatcaccaggtacaaagatgaatctagcaaggcccaggcagaagttgatcgactcttagaaatcttgaaggaggtggaaaatgagaagaatgacaaagataagaagatagctgagttggaaaggcaagtgaaagaccagaataagaaggtagcaaatctgaagcacaaggaacaggtggaaaaaaagaagagtgcacaaatgttagaggaggcgcgacgacgggaggacaatctcaacgacagctctcagcagctacaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 44 - DNA, 2874 bp, synthetic construct
  source 1..2874: mol_type = other DNA
atgtatggaagtgcccgctctgttgggaaggtggagccgagcagccagagccctgggcgttcacccaggcttccacgttcccctcgcttgggtcaccgtcgaaccaacagtacgggagggagttcgggaagcagtgttggaggtggcagtgggaaaaccctttcaatggaaaatatacaatctttaaatgctgcctatgccacctctggccctatgtatctaagtgaccatgaaaatgtgggttcagaaacacctaaaagcaccatgacacttggccgttctgggggacgtctgccttacggtgttcggatgactgctatgggtagtagccccaatatagctagcagtggggttgctagtgacaccatagcatttggagagcatcacctccctcctgtgagtatggcatccactgtacctcactcccttcgtcaggcgagagataacacaatcatggatctgcagacacagctgaaggaagtattaagagaaaatgatctcttgcggaaggatgtggaagtaaaggagagcaaattgagttcttcaatgaatagcatcaagaccttctggagcccagagctgaagaaggaacgagccctgagaaaagatgaagcttccaaaatcaccatttggaaggaacagtacagagttgtacaggaggaaaaccagcacatgcagatgacaatccaggctctccaggatgaattgcggatccagagggacctgaatcagctgtttcagcaggatagtagcagcaggactggcgaaccttgtgtagcagagctgacagaggagaactttcagaggcttcatgctgagcatgagcggcaggccaaagagctgtttcttcttcgaaagacattggaggaaatggagctgcgtattgagactcaaaagcagaccctaaatgctcgggatgaatccattaagaagcttctggaaatgttgcagagcaaaggactttctgccaaggctaccgaggaagaccatgagagaacaagacgactggcagaggcagagatgcacgttcatcacctagaaagccttttggagcagaaggaaaaagagaacagtatgttgagagaggagatgcatcgaaggtttgagaatgctcctgattctgccaaaacaaaagctctgcaaactgttattgagatgaaggattcaaaaatttcctctatggagcgtgggcttcgagacctggaagaggaaattcagatgctgaaatcgaatggtgctttgagtactgaggaaagggaagaagaaatgaagcaaatggaagtgtatcggagccattctaaatttatgaaaaataagattggccaggtgaaacaggagctgtccagaaaggacacagaactactcgccctgcagacaaagctagaaacactcacaaaccagttctcagatagtaaacagcacattgaagtgttgaaggagtccttgactgctaaggagcagagggctgccatcctgcagactgaggtggatgctctccgattgcgtttggaagagaaggaaaccatgttgaataaaaagacaaaacaaattcaggatatggctgaagagaaggggacacaagctggagagatacatgacctcaaggacatgttggatgtgaaggagcggaaggttaatgttcttcagaagaagattgaaaatcttcaagagcagcttagagacaaggaaaagcagatgagcagcttgaaagaacgggtcaaatccttgcaggctgacaccaccaacactgacactgccttgacaactttggaggaggcccttgcagagaaagaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 45 - DNA, 3743 bp, synthetic construct
  source 1..3743: mol_type = other DNA
atgtatggaagtgcccgctctgttgggaaggtggagccgagcagccagagccctgggcgttcacccaggcttccacgttcccctcgcttgggtcaccgtcgaaccaacagtacgggagggagttcgggaagcagtgttggaggtggcagtgggaaaaccctttcaatggaaaatatacaatctttaaatgctgcctatgccacctctggccctatgtatctaagtgaccatgaaaatgtgggttcagaaacacctaaaagcaccatgacacttggccgttctgggggacgtctgccttacggtgttcggatgactgctatgggtagtagccccaatatagctagcagtggggttgctagtgacaccatagcatttggagagcatcacctccctcctgtgagtatggcatccactgtacctcactcccttcgtcaggcgagagataacacaatcatggatctgcagacacagctgaaggaagtattaagagaaaatgatctcttgcggaaggatgtggaagtaaaggagagcaaattgagttcttcaatgaatagcatcaagaccttctggagcccagagctgaagaaggaacgagccctgagaaaagatgaagcttccaaaatcaccatttggaaggaacagtacagagttgtacaggaggaaaaccagcacatgcagatgacaatccaggctctccaggatgaattgcggatccagagggacctgaatcagctgtttcagcaggatagtagcagcaggactggcgaaccttgtgtagcagagctgacagaggagaactttcagaggcttcatgctgagcatgagcggcaggccaaagagctgtttcttcttcgaaagacattggaggaaatggagctgcgtattgagactcaaaagcagaccctaaatgctcgggatgaatccattaagaagcttctggaaatgttgcagagcaaaggactttctgccaaggctaccgaggaagaccatgagagaacaagacgactggcagaggcagagatgcacgttcatcacctagaaagccttttggagcagaaggaaaaagagaacagtatgttgagagaggagatgcatcgaaggtttgagaatgctcctgattctgccaaaacaaaagctctgcaaactgttattgagatgaaggattcaaaaatttcctctatggagcgtgggcttcgagacctggaagaggaaattcagatgctgaaatcgaatggtgctttgagtactgaggaaagggaagaagaaatgaagcaaatggaagtgtatcggagccattctaaatttatgaaaaataagattggccaggtgaaacaggagctgtccagaaaggacacagaactactcgccctgcagacaaagctagaaacactcacaaaccagttctcagatagtaaacagcacattgaagtgttgaaggagtccttgactgctaaggagcagagggctgccatcctgcagactgaggtggatgctctccgattgcgtttggaagagaaggaaaccatgttgaataaaaagacaaaacaaattcaggatatggctgaagagaaggggacacaagctggagagatacatgacctcaaggacatgttggatgtgaaggagcggaaggttaatgttcttcagaagaagattgaaaatcttcaagagcagcttagagacaaggaaaagcagatgagcagcttgaaagaacgggtcaaatccttgcaggctgacaccaccaacactgacactgccttgacaactttggaggaggcccttgcagagaaagagcggacaattgaacgcttaaaggagcagagggacagagatgagcgagagaagcaagaggaaattgataactacaaaaaagatcttaaagacttgaaggaaaaagtcagcctgttgcaaggcgacctttcagagaaagaggcttcacttttggatctgaaagagcatgcttcttctctggcatcctcaggactgaaaaaggactcacggcttaagacactagagattgctttggagcagaagaaggaggagtgtctgaaaatggaatcacaattgaaaaaggcacatgaggcagcattggaagccagagccagtccagagatgagtgaccgaatacagcacttggagagagagatcaccaggtacaaagatgaatctagcaaggcccaggcagaagttgatcgactcttagaaatcttgaaggaggtggaaaatgagaagaatgacaaagataagaagatagctgagttggaaaggcaagtgaaagaccagaataagaaggtagcaaatctgaagcacaaggaacaggtggaaaaaaagaagagtgcacaaatgttagaggaggcgcgacgacgggaggacaatctcaacgacagctctcagcagctacagatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 46 - DNA, 1842 bp, synthetic construct
  source 1..1842: mol_type = other DNA
atggcagtgcagctggtgcccgactcagctctcggcctgctgatgatgacggagggccgccgatgtcaagtacatcttcttgatgacaggaagctggaactcctagtacagcccaagctgttggccaaggagcttcttgaccttgtggcttctcacttcaatctgaaggaaaaggagtactttggaatagcattcacagatgaaacgggacacttaaactggcttcagctagatcgaagagtattggaacatgacttccctaaaaagtcaggacccgtggttttatacttttgtgtcaggttctatatagaaagcatttcatacctgaaggataatgctaccattgagcttttctttctgaacgcgaagtcctgcatctacaaggagcttattgacgttgacagcgaagtggtgtttgaattagcttcctatattttacaggaggcaaagggagatttttctagcaatgaagttgtgaggagtgacttgaagaagctgccagcccttcccacccaagccctgaaggagcacccttccctggcctactgtgaagacagagtcattgagcactacaagaaactgaacggtcagacaagaggtcaagcaatcgtaaactacatgagcatcgtggagtctctcccaacctacggggttcactattatgcagtgaaggacaagcagggcataccatggtggctgggcctgagctacaaagggatcttccagtatgactaccatgataaagtgaagccaagaaaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 47 - DNA, 2703 bp, synthetic construct
  source 1..2703: mol_type = other DNA
atggcggcgatggcgcctggaggtagtggcagtggtggcggcgtgaatccatttctcagtgattcggatgaggacgatgacgaggtagctgcaacagaggaacggcgggcagtacttcggctgggcgccggaagtggcctagatcctggctctgcgggctcgctgtcgccacaggatcccgtggccttaggaagcagtgcgcggccagggctccctggggaggcgtcggcggctgcagtggccctggggggcaccggggagaccccggcccgattatcaattgatgcgatcgctgctcagctgttgcgcgatcaatacttgctgaccgccctggagctgcataccgagctgttagagagtggccgggagctgcctcggctgcgcgactacttctccaatccaggcaacttcgagaggcaaagtggaaccccgccggggatgggggcgccaggggtccctggagcagccggcgttgggggcgctggaggtcgggaaccgagtacagcgtcgggcgggggacagctcaatcgagctgggagcattagtacccttgattctttagactttgcaagatattcagatgatggtaacagggaaacagatgaaaaagtggcagtcctggagtttgaactacggaaagccaaggagaccattcaggccctccgagccaacctgacaaaggccgcagaacatgaagttcctttacaggaacgaaaaaattacaaatcaagtcctgaaattcaggagccaatcaaacctcttgaaaagagagctctaaacttcttagtcaatgaatttttattgaagaataactataagcttacatcaataaccttttcagatgaaaacgatgatcaggattttgaattatgggatgatgtaggattaaacattccaaaacctccagacttattgcaactctaccgggattttggaaatcatcaagtaactggaaaagatcttgtagatgtggccagtggagtagaagaagatgaattagaggcccttacaccaattataagcaaccttcctccaactcttgaaactccccagcctgcagagaactccatgttagtacagaaattagaagataaaattagtttgttaaatagtgagaaatggtcattgatggagcaaatcagaagacttaaaagtgaaatggacttcctcaaaaatgaacactttgccatcccagcagtttgtgactctgttcagcctcctttggatcagttgccccacaaagactctgaggacagtggacagcatccagatgtaaatagttcagacaagggaaaaaacacagacatccatctttcaatatcagatgaagctgattccactattcctaaagagaattccccaaattcattccccaggagagaaagagaaggaatgccaccttcttctctatcaagtaaaaagacagttcattttgataaacctaataggaaattgtctcctgcattccatcaagcactactctctttttgtcgaatgtcagcagatagtcgtttaggatacgaggtgtctcgtattgcagacagtgaaaaaagcgttatgttaatgctgggacgctgcctgccacacattgttcccaatgtgctattggcaaagagagaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 48 - DNA, 2781 bp, synthetic construct
  source 1..2781: mol_type = other DNA
atgaataccttccaagaccagagtggcagctccagtaatagagaaccccttttgaggtgtagtgatgcacggagggacttggagcttgctattggtggagttctccgggctgaacagcaaattaaagataacttgcgagaggtcaaagctcagattcacagttgcataagccgtcacctggaatgtcttagaagccgtgaggtatggctgtatgaacaggtggaccttatttatcagcttaaagaggagacacttcaacagcaggctcagcagctctactcgttattgggccagttcaattgtcttactcatcaactggagtgtacccaaaacaaagatctagccaatcaagtctctgtgtgcctggagagactgggcagtttgacccttaagcctgaagattcaactgtcctgctctttgaagctgacacaattactctgcgccagaccatcaccacatttgggtctctcaaaaccattcaaattcctgagcacttgatggctcatgctagttcagcaaatattgggcccttcctggagaagagaggctgtatctccatgccagagcagaagtcagcatccggtattgtagctgtccctttcagcgaatggctccttggaagcaaacctgccagtggttatcaagctccttacatacccagcaccgacccccaggactggcttacccaaaagcagaccttggagaacagtcagacttcttccagagcctgcaatttcttcaataatgtcgggggaaacctaaagggcttagaaaactggctcctcaagagtgaaaaatcaagttatcaaaagtgtaacagccattccactactagttctttctccattgaaatggaaaaggttggagatcaagagcttcctgatcaagatgagatggacctatcagattggctagtgactccccaggaatcccataagctgcggaagcctgagaatggcagtcgtgaaaccagtgagaagtttaagctcttattccagtcctataatgtgaatgattggcttgtcaagactgactcctgtaccaactgtcagggaaaccagcccaaaggtgtggagattgaaaacctgggcaatctgaagtgcctgaatgaccacttggaggccaagaaaccattgtccacccccagcatggttacagaggattggcttgtccagaaccatcaggacccatgtaaggtagaggaggtgtgcagagccaatgagccctgcacaagctttgcagagtgtgtgtgtgatgagaattgtgagaaggaggctctgtataagtggcttctgaagaaagaaggaaaggataaaaatgggatgcctgtggaacccaaacctgagcctgagaagcataaagattccctgaatatgtggctctgtcctagaaaagaagtaatagaacaaactaaagcaccaaaggcaatgactccttctagaattgctgattccttccaagtcataaagaacagccccttgtcggagtggcttatcaggcccccatacaaagaaggaagtcccaaggaagtgcctggtactgaagacagagctggcaaacagaagtttaaaagccccatgaatacttcctggtgttcctttaacacagctgactgggtcctgccaggaaagaagatgggcaacctcagccagttatcttctggagaagacaagtggctgcttcgaaagaaggcccaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 49 - DNA, 1919 bp, synthetic construct
  source 1..1919: mol_type = other DNA
atgaaagtgaccgtgtgcttcggacggacccgggtggtcgtgccgtgcggggacggccacatgaaagttttcagcctcatccagcaggcggtgacccgctaccggaaggccatcgccaaggatccaaactactggatacaggtgcatcgcttggaacatggagatggaggaatactagaccttgatgacattctttgtgatgtagcagacgataaagacagaatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 50 - DNA, 1665 bp, synthetic construct
  source 1..1665: mol_type = other DNA
atgaaagtgaccgtgtgcttcggacggacccgggtggtcgtgccgtgcggggacggccacatgaaagttttcagcctcatccagcaggcggtgacccgctaccggaaggccatcgccaaggatccaaactactggatacaggtgcatcgcttggaacatggagatggaggaatactagaccttgatgacattctttgtgatgtagcagacgataaagacagactggtagcagtgtttgatgagcaggatccacatcacggaggtgatggcaccagtgccagttccacgggtacccagagcccagagatatttggtagtgagcttggcaccaacaatgtctcagcctttcagccttaccaagcaacaagtgaaattgaggtcacaccttcagtccttcgagcaaatatgcctcttcatgttcgacgcagtagtgacccagctctaattggcctctccacttctgtcagtgatagtaatttttcctctgaagagccttcaaggaaaaatcccacacgctggtcaacaacagctggcttcctcaagcagaacactgctgggagtcctaaaacctgcgacaggaaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 51 - DNA, 1791 bp, synthetic construct
  source 1..1791: mol_type = other DNA
atggagtctggcagtaccgccgccagtgaggaggcacgcagccttcgagaatgtgagctctacgtccagaagcataacattcaagcgctgctcaaagattctattgtgcagttgtgcactgctcgacctgagagacccatggcattcctcagggaatactttgagaggttggagaaggaggaggcaaaacagattcagaatctgcagaaagcaggcactcgtacagactcaagggaggatgagatttctcctcctccacccaacccagtggttaaaggtaggaggcgacgaggtgctatcagcgctgaggtctacacggaggaagatgcggcatcctatgttagaaaggttataccaaaagattacaagacaatggccgctttagccaaagccattgaaaagaatgtgctgttttcacatcttgatgataatgagagaagtgatatttttgatgccatgttttcggtctcctttatcgcaggagagactgtgattcagcaaggtgatgaaggggataacttctatgtgattgatcaaggagagacggatgtctatgttaacaatgaatgggcaaccagtgttggggaaggagggagctttggagaacttgctttgatttatggaacaccgagagcagccactgtcaaagcaaagacaaatgtgaaattgtggggcatcgaccgagacagctatagaagaatcctcatggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 52 - DNA, 3529 bp, synthetic construct
  source 1..3529: mol_type = other DNA
atgggcaccttgcgggatttacagtacgcgctccaggagaagatcgaggagctgaggcagcgggatgctctcatcgacgagctggagctggagttggatcagaaggacgaactgatccagaagctgcagaacgagctggacaagtaccgctcggtgatccgaccagccacccagcaggcgcagaagcagagcgcgagcaccttgcagggcgagccgcgcaccaagcggcaggcgatctccgccgagcccaccgccttcgacatccaggatctcagccatgtgaccctgcccttctaccccaagagcccacagtccaaggatcttataaaggaagctatccttgacaatgactttatgaagaacttggagctgtcgcagatccaggagattgtggattgtatgtacccggtggagtatggcaaggacagttgcatcatcaaagaaggagacgtggggtcactggtgtatgtcatggaagatggtaaggttgaagttacaaaagaaggtgtgaagttgtgtaccatgggtccaggaaaagtgtttggggaattggctattctttacaactgtacccggacagcgaccgtcaagactcttgtaaatgtaaaactctgggccattgatcgacaatgttttcaaacaataatgatgaggacaggactcatcaagcataccgagtatatggaatttttaaaaagcgttccaacattccagagccttcctgaagagatcctcagcaagcttgctgatgtccttgaagagacccactatgaaaatggagaatatattatcaggcaaggtgcaagaggggacaccttctttatcatcagcaaaggaacggtaaatgtcactcgtgaagactcaccgagtgaagacccagtctttcttagaactttaggaaaaggagactggtttggagagaaagccttgcaggggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 53 - DNA, 1042 bp, synthetic construct
  source 1..1042: mol_type = other DNA
atgggcaccttgcgggatttacagtacgcgctccaggagaagatcgaggagctgaggcagcgggatgctctcatcgacgagctggagctggagttggatcagaaggacgaactgatccagaagctgcagaacgagctggacaagtaccgctcggtgatccgaccagccacccagcaggcgcagaagcagagcgcgagcaccttgcagggcgagccgcgcaccaagcggcaggcgatctccgccgagcccaccgccttcgacatccaggatctcagccatgtgaccctgcccttctaccccaagagcccacagtccaaggatcttataaaggaagctatccttgacaatgactttatgaagaacttggagctgtcgcagatccaggagattgtggattgtatgtacccggtggagtatggcaaggacagttgcatcatcaaagaaggagacgtggggtcactggtgtatgtcatggaagatggtaaggttgaagttacaaaagaaggtgtgaagttgtgtaccatgggtccaggaaaagtgtttggggaattggctattctttacaactgtacccggacagcgaccgtcaagactcttgtaaatgtaaaactctgggccattgatcgacaatgttttcaaacaataatgatgaggacaggactcatcaagcataccgagtatatggaatttttaaaaagcgttccaacattccagagccttcctgaagagatcctcagcaagcttgctgatgtccttgaagaggtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 54 - DNA, 2010 bp, synthetic construct
  source 1..2010: mol_type = other DNA
atgacttttcaggtttgggggtggcggagggaagacgcatctcaggttttggcctgggtgcctgacgccgagggcgggagacggggaatgctgacgcgcaggagcctggctacaaaagaccccacagctgtagagagagcaaacttgttaaacatggctaaactgagtatcaaaggactcattgaatctgctctgagctttggccgcactttggattctgactatccccccttgcagcaattctttgttgttatggaacattgcctgaaacacggtcttaaagtaagaaaatcatttttgagttacaacaaaaccatctggggccctttggaactggtggagaagctgtaccccgaagcagaggaaataggagctagtgtccgggatctacctggtctgaagacccctctgggtcgagcaagagcgtggcttcgattagccctcatgcaaaaaaaaatggccgattacttacgttgcttaattattcagagggatctcttgagtgagttttatgagtatcacgcactaatgatggaagaagaaggagcagtaattgttgggctgctggttggcctgaatgtgatcgatgctaatctgtgtgtgaagggagaggatttagactcacaagttggagtgattgatttttctatgtatttaaagaatgaagaagatattggaaataaagaaaggaatgttcaaattgctgccatattagaccaaaagaattatgttgaagaattaaatagacaactgaacagcacagtcagcagcctccattcaagagttgattcattagaaaagtcaaatactaagctgattgaagagttagcaatagcaaagaataacatcattaaactccaggaagaaaatcatcaattacgaagtgaaaataaattgattttaatgaaaacacagcagcacctagaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 55 - DNA, 1476 bp, synthetic construct
  source 1..1476: mol_type = other DNA
atgacccagttcctgccgcccaaccttctggccctctttgccccccgtgaccctattccatacctgccacccctggagaaactgccacatgaaaaacaccacaatcaaccttattgtggcattgcgccgtacattcgagagtttgaggaccctcgagatgcccctcctccaactcgtgctgaaacccgagaggagcgcatggagaggaaaagacgggaaaagattgagcggcgacagcaagaagtggagacagagcttaaaatgtgggaccctcacaatgatcccaatgctcagggggatgccttcaagactctcttcgtggcgagagtgaattatgacacaacagaatccaagctccggagagagtttgaggtgtacggacctatcaaaagagaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 56 - DNA, 2094 bp, synthetic construct
  source 1..2094: mol_type = other DNA
atggcgtcgctcaccgtgaaggcctaccttctgggcaaggaggacgcggcgcgcgagattcgccgcttcagcttctgctgcagccccgagcctgaggcggaagccgaggctgcggcgggtccgggaccctgcgagcggctgctgagccgggtggccgccctgttccccgcgctgcggcctggcggcttccaggcgcactaccgcgatgaggacggggacttggttgccttttccagtgacgaggaattgacaatggccatgtcctacgtgaaggatgacatcttccgaatctacattaaagagaaaaaagagtgccggcgggaccaccgcccaccgtgtgctcaggaggcgccccgcaacatggtgcaccccaatgtgatctgcgatggctgcaatgggcctgtggtaggaacccgctacaagtgcagcgtctgcccagactacgacttgtgtagcgtctgcgagggaaagggcttgcaccgggggcacaccaagctcgcattccccagccccttcgggcacctgtctgagggcttctcgcacagccgctggctccggaaggtgaaacacggacacttcgggtggccaggatgggaaatgggtccaccaggaaactggagcccacgtcctcctcgtgcaggggaggcccgccctggccccacggcagaatcagcttctggtccatcggaggatccgagtgtgaatttcctgaagaacgttggggagagtgtggcagctgcccttagccctctggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 57 - DNA, 2478 bp, synthetic construct
  source 1..2478: mol_type = other DNA
atggaagggagaggaccgtaccggatctacgaccctgggggcagcgtgccctcaggagaggcatccgcagcttttgagcgcctagtgaaggagaattcccggctgaaggaaaaaatgcaagggataaagatgttaggggagcttttggaagagtcccagatggaagcgaccaggctccggcagaaggcagaggagctagtgaaggacaacgagctgctcccaccaccttctccctccttgggctccttcgaccccctggctgagctcacaggaaaggactcaaatgtcacagcatctcccacagcccctgcatgccccagtgacaagccagcaccagtccagaagcctccatccagtggcacctcctctgaatttgaagtggtcactcctgaggagcagaattcaccagagagcagcagccatgccaatgcgatggcgctgggccccctgccccgtgaggacggcaacctgatgctgcacctgcagcgcctggagaccacgctgagtgtgtgtgccgaggagccggaccacggccagctcttcacccacctgggccgcatggccctggagttcaaccgactggcatccaaggtgcacaagaatgagcagcgcacctccattctgcagaccctgtgtgagcagcttcggaaggagaacgaggctctgaaggccaagttggataagggcctggaacagcgggatcaggctgccgagaggctgcgggaggaaaatttggagctcaagaagttgttgatgagcaatggcaacaaagagggtgcgtctgggcggccaggctcaccgaagatggaagggacaggcaagaaggcagtggctggacagcagcaggctagtgtgacggcaggtaaggtcccagaggtggtggccttgggcgcagccgagaagaaggtgaagatgctggagcagcagcgcagtgagctgctggaagtgaacaagcagtgggaccagcatttccggtccatgaagcagcagtatgagcagaagatcactgagctgcgtcagaagctggctgatttgcagaagcaggtgactgacctggaggccgagcgggagcagaagcagcgtgactttgaccgcaagctcctcctggccaagtccaagattgaaatggaggagaccgacaaggagcagctgacagcagaggccaaggagctgcgccaaaaggtcaagtacctgcaggatcagctgagcccactcacccgacagcgtgagtaccaggaaaaggagatccagcggctcaacaaggccctggaggaagcactgagcatccaaaccccgccatcatctccaccaacagcatttgggagcccagaaggagcaggggccctcctaaggaaacaggagctggtcacgcagaatgagttgctgaaacagcaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 58 - DNA, 2862 bp, synthetic construct
  source 1..2862: mol_type = other DNA
atggaagggagaggaccgtaccggatctacgaccctgggggcagcgtgccctcaggagaggcatccgcagcttttgagcgcctagtgaaggagaattcccggctgaaggaaaaaatgcaagggataaagatgttaggggagcttttggaagagtcccagatggaagcgaccaggctccggcagaaggcagaggagctagtgaaggacaacgagctgctcccaccaccttctccctccttgggctccttcgaccccctggctgagctcacaggaaaggactcaaatgtcacagcatctcccacagcccctgcatgccccagtgacaagccagcaccagtccagaagcctccatccagtggcacctcctctgaatttgaagtggtcactcctgaggagcagaattcaccagagagcagcagccatgccaatgcgatggcgctgggccccctgccccgtgaggacggcaacctgatgctgcacctgcagcgcctggagaccacgctgagtgtgtgtgccgaggagccggaccacggccagctcttcacccacctgggccgcatggccctggagttcaaccgactggcatccaaggtgcacaagaatgagcagcgcacctccattctgcagaccctgtgtgagcagcttcggaaggagaacgaggctctgaaggccaagttggataagggcctggaacagcgggatcaggctgccgagaggctgcgggaggaaaatttggagctcaagaagttgttgatgagcaatggcaacaaagagggtgcgtctgggcggccaggctcaccgaagatggaagggacaggcaagaaggcagtggctggacagcagcaggctagtgtgacggcaggtaaggtcccagaggtggtggccttgggcgcagccgagaagaaggtgaagatgctggagcagcagcgcagtgagctgctggaagtgaacaagcagtgggaccagcatttccggtccatgaagcagcagtatgagcagaagatcactgagctgcgtcagaagctggctgatttgcagaagcaggtgactgacctggaggccgagcgggagcagaagcagcgtgactttgaccgcaagctcctcctggccaagtccaagattgaaatggaggagaccgacaaggagcagctgacagcagaggccaaggagctgcgccaaaaggtcaagtacctgcaggatcagctgagcccactcacccgacagcgtgagtaccaggaaaaggagatccagcggctcaacaaggccctggaggaagcactgagcatccaaaccccgccatcatctccaccaacagcatttgggagcccagaaggagcaggggccctcctaaggaaacaggagctggtcacgcagaatgagttgctgaaacagcaggtgaagatcttcgaggaggacttccagagggagcgcagtgatcgtgagcgcatgaatgaggagaaggaagagctgaagaagcaagtggagaagctgcaggcccaggtcaccctgtcaaatgcccagctaaaagcattcaaagatgaggagaaggcaagagaagccctcagacagcagaagaggaaagcaaaggcctcaggagagcgttaccatgtggagccccacccagaacatctctgcggggcctacccctacgcctacccgcccatgccagccatggtgccacaccatggcttcgaggactggtcccagatccgctacccccctccccccatggccatggagcacccgcccccactccccaactcgcgcctcttccatctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 59 - DNA, 2346 bp, synthetic construct
  source 1..2346: mol_type = other DNA
atggaagggagaggaccgtaccggatctacgaccctgggggcagcgtgccctcaggagaggcatccgcagcttttgagcgcctagtgaaggagaattcccggctgaaggaaaaaatgcaagggataaagatgttaggggagcttttggaagagtcccagatggaagcgaccaggctccggcagaaggcagaggagctagtgaaggacaacgagctgctcccaccaccttctccctccttgggctccttcgaccccctggctgagctcacaggaaaggactcaaatgtcacagcatctcccacagcccctgcatgccccagtgacaagccagcaccagtccagaagcctccatccagtggcacctcctctgaatttgaagtggtcactcctgaggagcagaattcaccagagagcagcagccatgccaatgcgatggcgctgggccccctgccccgtgaggacggcaacctgatgctgcacctgcagcgcctggagaccacgctgagtgtgtgtgccgaggagccggaccacggccagctcttcacccacctgggccgcatggccctggagttcaaccgactggcatccaaggtgcacaagaatgagcagcgcacctccattctgcagaccctgtgtgagcagcttcggaaggagaacgaggctctgaaggccaagttggataagggcctggaacagcgggatcaggctgccgagaggctgcgggaggaaaatttggagctcaagaagttgttgatgagcaatggcaacaaagagggtgcgtctgggcggccaggctcaccgaagatggaagggacaggcaagaaggcagtggctggacagcagcaggctagtgtgacggcaggtaaggtcccagaggtggtggccttgggcgcagccgagaagaaggtgaagatgctggagcagcagcgcagtgagctgctggaagtgaacaagcagtgggaccagcatttccggtccatgaagcagcagtatgagcagaagatcactgagctgcgtcagaagctggctgatttgcagaagcaggtgactgacctggaggccgagcgggagcagaagcagcgtgactttgaccgcaagctcctcctggccaagtccaagattgaaatggaggagaccgacaaggagcagctgacagcagaggccaaggagctgcgccaaaaggtcaagtacctgcaggatcagctgagcccactcacccgacagcgtgagtaccaggaaaaggagatccagcggctcaacaaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 60 - DNA, 1830 bp, synthetic construct
  source 1..1830: mol_type = other DNA
atggcctccgggagtgtggccgagtgcctgcagcaggagaccacctgccccgtgtgcctgcagtacttcgcagagcccatgatgctcgactgcggccataacatctgttgcgcgtgcctcgcccgctgctggggcacggcagagactaacgtgtcgtgcccgcagtgccgggagaccttcccgcagaggcacatgcggcccaaccggcacctggccaacgtgacccaactggtaaagcagctgcgcaccgagcggccgtcggggcccggcggcgagatgggcgtgtgcgagaagcaccgcgagcccctgaagctgtactgcgaggaggaccagatgcccatctgcgtggtgtgcgaccgctcccgcgagcaccgcggccacagcgtgctgccgctcgaggaggcggtggagggcttcaaggagcaaatccagaaccagctcgaccatttaaaaagagtgaaagatttaaagaagagacgtcgggcccagggggaacaggcacgagctgaactcttgagcctaacccagatggagagggagaagattgtttgggagtttgagcagctgtatcactccttaaaggagcatgagtatcgcctcctggcccgccttgaggagctagacttggccatctacaatagcatcaatggtgccatcacccagttctcttgcaacatctcccacctcagcagcctgatcgctcagctagaagagaagcagcagcagcccaccagggagctcctgcaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 61 - DNA, 2943 bp, synthetic construct
  source 1..2943: mol_type = other DNA
atggcggaaaacaaaggcggcggcgaggctgagagcggcggcgggggcagcggcagcgcgccggtaactgccggggccgccgggcccgccgcgcaggaggcggagccgcctctcaccgcggtgctggtggaggaggaggaggaggaaggcggcagggccggcgctgagggcggcgcggccgggcccgacgacgggggggtggccgcggcctcctcgggctcggcccaggctgcttcatctcctgcggcctcagtgggcactggagttgccgggggcgcagtatcgacgccggctccagctccagcctcggctcccgctccgggtccctcggcagggccgcctcctggaccgccagcctcgctcctggacacctgcgccgtgtgtcagcagagcttgcagagccggcgtgaggcggagcccaagctgctgccctgtcttcactccttctgcctgcgctgcctgcccgagccggagcgccagctcagcgtgcccatcccggggggcagcaacggcgacatccagcaagttggtgtaatacggtgcccagtatgccgccaagaatgcagacagatagaccttgtggataattattttgtgaaagacacatctgaagctcctagcagttctgatgaaaaatcagaacaggtatgtactagttgtgaagacaatgcaagtgcagttggcttttgtgtagaatgtggagagtggctatgtaagacatgtatcgaagcacatcaaagagtaaaatttactaaagatcacttgatcaggaagaaagaagatgtctcagagtctgttggagcatctggtcaacgccctgttttctgccctgtacacaaacaagaacagttgaaacttttctgtgaaacatgtgatagattgacatgtagagactgtcagctattggaacacaaagaacataggtatcagtttttggaagaagcttttcaaaatcagaagggtgcaattgagaatctactggcgaaacttcttgagaagaagaattatgttcattttgcagctactcaggtgcagaataggataaaagaagtaaatgagactaacaaacgagtagaacaggaaattaaagtggccattttcacccttatcaatgaaattaataagaaaggaaaatctctcttacaacagctagagaatgttacaaaggaaagacagatgaagttactacagcagcagaatgacatcacaggcctttcccggcaggtgaagcatgttatgaacttcacaaattgggcaattgcaagtggcagcagcacagcactactatacagcaagcgactgattactttccagttgcgtcatattttgaaagcacggtgtgatcctgtccctgctgctaatggagcaatacgtttccattgtgatcccaccttctgggcaaagaatgtagtcaatttaggtaatctagtaatagagagtaaaccagctcctggttatactcctaatgttgtagttgggcaagttcctccagggacaaaccacattagtaaaacccctggacagattaacttagcacagcttcgactccagcacatgcaacaacaagtatatgcacagaaacatcagcagttgcaacagatgaggatgcagcaaccaccagcacctgtaccaactacaacaacaacaacacaacagcatcctagacaagcagcccctcagatgttacaacaacagcctcctcgattgatcagtgtgcaaacaatgcaaagaggcaacatgaactgtggagcttttcaagcccatcagatgagactggctcagaatgctgccagaataccagggatacccaggcacagcggccctcaatattccatgatgcagccacacctccaaagacaagaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 62 - DNA, 1922 bp, synthetic construct
  source 1..1922: mol_type = other DNA
atggcggacagcgccagcgagagcgacacggacggggcggggggcaacagcagcagctcggccgccatgcagtcgtcctgctcgtcgacctcgggcggcggcggtggcggcgggggaggcggcggcggtgggaagtcggggggcattgtcatctcgccgttccgcctggaggagctcaccaaccgcctggcctcgctgcagcaagagaacaaggtgctgaagatagagctggagacctacaaactgaagtgcaaggcactgcaggaggagaaccgcgacctgcgcaaagccagcgtgaccatccaagccagggctgagcaggaagaagaattcattagtaacactttattcaagaaaattcaggctttgcagaaggagaaagaaacccttgctgtaaattatgagaaagaagaagaattcctcactaatgagctctccagaaaattgatgcagttgcagcatgagaaagccgaactagaacagcatcttgaacaagagcaggaatttcaggtcaacaaactgatgaagaaaattaaaaaactggagaatgacaccatttctaagcaacttacattagaacagatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 63 - DNA, 3774 bp, synthetic construct
  source 1..3774: mol_type = other DNA
atggaggtggcggtggagaaggcggtggcggcggcggcagcggcctcggctgcggcctccggggggccctcggcggcgccgagcggggagaacgaggccgagagtcggcagggcccggactcggagcgcggcggcgaggcggcccggctcaacctgttggacacttgcgccgtgtgccaccagaacatccagagccgggcgcccaagctgctgccctgcctgcactctttctgccagcgctgcctgcccgcgccccagcgctacctcatgctgcccgcgcccatgctgggctcggccgagaccccgccacccgtccctgcccccggctcgccggtcagcggctcgtcgccgttcgccacccaagttggagtcattcgttgcccagtttgcagccaagaatgtgcagagagacacatcatagataacttttttgtgaaggacactactgaggttcccagcagtacagtagaaaagtcaaatcaggtatgtacaagctgtgaggacaacgcagaagccaatgggttttgtgtagagtgtgttgaatggctctgcaagacgtgtatcagagctcatcagagggtaaagttcacaaaagaccacactgtcagacagaaagaggaagtatctccagaggcagttggtgtcaccagccagcgaccagtgttttgtccttttcataaaaaggagcagctgaagctgtactgtgagacatgtgacaaactgacatgtcgagactgtcagttgttagaacataaagagcatagataccaatttatagaagaagcttttcagaatcagaaagtgatcatagatacactaatcaccaaactgatggaaaaaacaaaatacataaaattcacaggaaatcagatccaaaacagaattattgaagtaaatcaaaatcaaaagcaggtggaacaggatattaaagttgctatatttacactgatggtagaaataaataaaaaaggaaaagctctactgcatcagttagagagccttgcaaaggaccatcgcatgaaacttatgcaacaacaacaggaagtggctggactctctaaacaattggagcatgtcatgcatttttctaaatgggcagtttccagtggcagcagtacagcattactttatagcaaacgactgattacataccggttacggcacctccttcgtgcaaggtgtgatgcatccccagtgaccaacaacaccatccaatttcactgtgatcctagtttctgggctcaaaatatcatcaacttaggttctttagtaatcgaggataaagagagccagccacaaatgcctaagcagaatcctgtcgtggaacagaattcacagccaccaagtggtttatcatcaaaccagttatccaagttcccaacacagatcagcctagctcaattacggctccagcatatgcagcaacagcaaccgcctccacgtttgataaactttcagaatcacagccccaaacccaatggaccagttcttcctcctcatcctcaacaactgagatatccaccaaaccagaacataccacgacaagcaataaagccaaaccccctacagatggctttcttggctcaacaagccataaaacagtggcagatcagcagtggacagggaaccccatcaactaccaacagcacatcctctactccttccagccccacgattactagtgcagcaggatatgatggaaaggcttttggttcacctatgatcgatttgagctcaccagtgggagggtcttataatcttccctctcttccggatattgactgttcaagtactattatgctggacaatattgtgaggaaagatactaatatagatcatggccagccaagaccaccctcaaacagaacggtccagtcaccaaattcatcagtgccatctccaggccttgcaggacctgttactatgactagtgtacaccccccaatacgttcacctagtgcctccagcgttggaagccgaggaagctctggctcttccagcaaaccagcaggagctgactctacacacaaagtcccagtggtcatgctggagccaattcgaataaaacaagaaaacagtggaccaccggaaaattatgatttccctgttgttatagtgaagcaagaatcagatgaagaatctaggcctcaaaatgccaattatccaagaagcatactcacctccctgctcttaaatagcagtcagagctctacttctgaggagactgtgctaagatcagatgcccctgatagtacaggagatcaacctggacttcaccaggacaattcctcaaatggaaagtctgaatggttggatccttcccagaagtcacctcttcatgttggagagacaaggaaagaggatgaccccaatgaggactggtgtgcagtttgtcaaaacggaggggaactcctctgctgtgaaaagtgccccaaagtattccatctttcttgtcatgtgcccacattgacaaattttccaagtggagagtggatttgcactttctgccgagacttatctaaaccagaagttgaatatgattgtgatgctcccagtcacaactcagaaaaaaagaaaactgaaggccttgttaagttaacacctatagataaaaggaagtgtgagcgcctacttttatttctttactgccatgaaatgagcctggcttttcaagaccctgttcctctaactgaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 64 - DNA, 1602 bp, synthetic construct
  source 1..1602: mol_type = other DNA
atggcggcgacggcggccgcagtggtggccgaggaggacacggagctgcgggacctgctggtgcagacgctggagaacagcggggtcctgaaccgcatcaaggctgaactccgagcagctgtgtttttagcactagaggagcaagaaaaagtagagaacaaaactcctttagttaatgagagcctgaaaaagtttttaaataccaaagacggtcgtttagtggctagtcttgttgcagaatttcttcagttttttaaccttgactttactttggctgtttttcaacctgaaactagcacactgcaaggtctcgaaggtcgagagaatttagcccgagatttaggtataattgaagcagaaggtactgtgggtggacccttattattagaagtgatcaggcgctgtcaacagaaagaaaaagggccaaccactggggaaggtgcacttgatctatctgatgtacattctccaccaaagtcaccagagggaaaaacaagtgcacagacaacaccaagtaaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 65 - DNA, 3317 bp, synthetic construct
  source 1..3317: mol_type = other DNA
atgtgcactgctctgagcccaaaggtacgcagtggacctggcctctctgatatgcatcagtatagccaatggctagccagcagacatgaagctaatttgctaccaatgaaagaagatctggccttgtggttaaccaatctattagggaaggagattacagcagaaacttttatggagaagttggacaatggtgccttgctctgtcaacttgcagaaactatgcaggagaaattcaaggagagcatggatgctaacaagcccacaaagaatctaccgttgaagaagatcccatgcaaaaccagtgcaccctcgggctccttttttgccagagacaatacagcaaatttcttatcctggtgccgagatttaggggtggatgaaacgtgtctatttgaatcggaaggtttggtcctccacaagcaacccagagaagtgtgtctctgtctgctagagcttggccggattgcagccaggtatggtgtggagcctcctggtttgataaagctggaaaaagagattgaacaagaagaaacactttctgccccttctccttcaccttctccttcatcaaagtcttctggaaaaaagagtacaggaaacttactggatgatgcagtgaaacgaatttctgaagatcctccttgcaaatgcccaaacaagttctgtgtggagcggctctcccaaggaagataccgagtgggagaaaagatcctcttcattagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 66 - DNA, 3030 bp, synthetic construct
  source 1..3030: mol_type = other DNA
atggaggagacgcagccgccgccgcagcctaagctgcccctgtgcgacagcctcatgatctggctgcagacattcaatactgcctcaccttgtcaagatgtcaaacagctgactagtggagttgccatggcacaagttcttcatcaaattgatgcagcttggtttaacgaatcttggttaagccgaattaaagaggatgttggggacaactggagaataaaggccagtaatgtaaagaaggtccttcaaggaattatgagttattatcatgagtttttggggcagcagatttcagaagcacttatccctgatttaaaccaaataaccgaatgttcagatccagtggagcttgggaggttgctccagcttattttaggttgtgcgatcaactgtgaaaagaagcaagaacatattcaaaatataatgacactggaagagtctgttcaacatgtggtcatgactgctattcaagagttgatgagtaaagaaatattgagctctcctccaaatgatgctgttggagaattggagcaacagcttaaaagagccttggaagaacttcaggaagcactagcagaaaaagaagagctgaggcaaagatgtgaagaattggatatgcaggtgactacacttcaagatgaaaagaattcactggtttctgaaaatgagatgatgaatgaaaaacttgaccagttggatggctcttttgatgatccaaacacagtggttgcaaaaaagtattttcatgcacaattacaactagaacaattacaggaagaaaacttcaggcttgaagctgcaaaagatgattaccgtgttcactgtgaagaacttgaaaagcagctaatcgaattccagcataggaatgatgaattgactagtcttgcagaagaaacaagagccctgaaagatgaaatagatgttcttagggctacctctgataaagcaaataaactggagtcaacagttgagatatatcgtcagaagctacaagatctgaatgaccttcgcaagcaggtgaaaactttacaggaaaccaacatgatgtatatgcataatacagtcagcttagaagaagaattaaaaaaagcaaatgcagcacgtacacaattagaaacatacaaaaggcaggttcaagatcttcatgttaaactttcctccgaatccaagagggcagacacactagcgtttgaaatgaagcggcttgaagaaaaacatgaagctttacttaaggaaaaagagagactaattgagcagcgtgatactttgaaagaaacaaatgaagagcttcgatgttcacaagtacaacaggaccacctaaaccaaacagatgcatctgctacaaaaagttatgagaatcttgctgctgagattatgccagtggaatatagggaggtgtttattcgactgcaacatgaaaataagatgcttcgcttacagcaagaaggctctgagaatgaacgtattgaggaacttcaggagcagctagaacagaaacaccgtaaaatgaatgaactggaaactgagcagaggctgagcaaagagcgtattagagaattgcagcagcagattgaggacctccagaaatctttacaggaacaaggttccaagtctgaaggcgaaagttccagcaaattaaagcagaagttggaagctcatatggaaaaactcacagaggtccatgaagaattacagaagaaacaagaactcattgaagatcttcagccagatataaatcaaaatgtacaaaagatcaatgaacttgaagctgctcttcagaagaaagatgaagatatgaaagcaatggaggaaagatataaaatgtacttggagaaagccagaaatgtaataaaaactttggatcccaagttaaatccagcatcagctgaaataatgctactaagaaagcagttggcagagaaagagagaagaattgagattctggaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 67 - skipped

SEQ ID NO: 68 - protein, 823 aa, synthetic construct
MLSRLSGLANVVLHELSGDDDTDQNMRAPLDPELHQESDMEFNNTTQEDVQERLAYAEQLVVELKDIIRQKDVQLQQKDEALQEERKAADNKIKKLKLHAKAKLTSLNKYIEEMKAQGGTVLPTEPQSEEQLSKHDKSSTEEEMEIEKIKHKLQEKEELISTLQAQLTQAQAEQPAQSSTEMEEFVMMKQQLQEKEEFISTLQAQLSQTQAEQAAQQVVREKDARFETQVRLHEDELLQLVTQADVETEMQQKLRVLQRKLEEHEESLVGRAQVVDLLQQELTAAEQRNQILSQQLQQMEAEHNTLRNTVETEREESKILLEKMELEVAERKLSFHNLQEEMHHLLEQFEQAGQAQAELESRYSALEQKHKAEMEEKTSHILSLQKTGQELQSACDALKDQNSKLLQDKNEQAVQSAQTIQQLEDQLQQKSKEISQFLNRLPLQQHETASQTSFPDVYNEGTQEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 69 - protein, 661 aa, synthetic construct
MPHLMERMVGSGLLWLALVSCILTQASAVQRGYGNPIEASSYGLDLDCGAPGTPEAHVCFDPCQNYTLLDEPFRSTENSAGSQGCDKNMSGWYRFVGEGGVRMSETCVQVHRCQTDAPMWLNGTHPALGDGITNHTACAHWSGNCCFWKTEVLVKACPGGYHVYRLEGTPWCNLRYCTDPSTVEDKCEKACRPEEECLALNSTWGCFCRQDLNSSDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 70 - protein, 112 aa, synthetic construct
MSSSSPPAGAASAAISASEKVDGFTRKSVRKAQRQKRSQGSSQFRSQGSQAELHPLPQLKGMSDPNWPGESPVPLTRADGTNTGFPRYPNDSVYANWMLSPSAAKLMDTFDS

SEQ ID NO: 71 - skipped

SEQ ID NO: 72 - protein, 1859 aa, synthetic construct
MVQLRKLLRVLTLMKFPCCVLEVLLCALAAAARGQEMYAPHSIRIEGDVTLGGLFPVHAKGPSGVPCGDIKRENGIHRLEAMLYALDQINSDPNLLPNVTLGARILDTCSRDTYALEQSLTFVQALIQKDTSDVRCTNGEPPVFVKPEKVVGVIGASGSSVSIMVANILRLFQIPQISYASTAPELSDDRRYDFFSRVVPPDSFQAQAMVDIVKALGWNYVSTLASEGSYGEKGVESFTQISKEAGGLCIAQSVRIPQERKDRTIDFDRIIKQLLDTPNSRAVVIFANDEDIKQILAAAKRADQVGHFLWVGSDSWGSKINPLHQHEDIAEGAITIQPKRATVEGFDAYFTSRTLENNRRNVWFAEYWEENFNCKLTISGSKKEDTDRKCTGQERIGKDSNYEQEGKVQFVIDAVYAMAHALHHMNKDLCADYRGVCPEMEQAGGKKLLKYIRNVNFNGSAGTPVMFNKNGDAPGRYDIFQYQTTNTSNPGYRLIGQWTDELQLNIEDMQWGKGVREIPASVCTLPCKPGQRKKTQKGTPCCWTCEPCDGYQYQFDEMTCQHCPYDQRPNENRTGCQDIPIIKLEWHSPWAVIPVFLAMLGIIATIFVMATFIRYNDTPIVRASGRELSYVLLTGIFLCYIITFLMIAKPDVAVCSFRRVFLGLGMCISYAALLTKTNRIYRIFEQGKKSVTAPRLISPTSQLAITSSLISVQLLGVFIWFGVDPPNIIIDYDEHKTMNPEQARGVLKCDITDLQIICSLGYSILLMVTCTVYAIKTRGVPENFNEAKPIGFTMYTTCIVWLAFIPIFFGTAQSAEKLYIQTTTLTISMNLSASVALGMLYMPKVYIIIFHPELNVQKRKRSFKAVVTAATMSSRLSHKPSDRPNGEAKTELCENVDPNNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 73 - skipped

SEQ ID NO: 74 - protein, 2142 aa, synthetic construct
MAAVTMSVPGRKAPPRPGPVPEAAQPFLFTPRGPSAGGGPGSGTSPQVEWTARRLVWVPSELHGFEAAALRDEGEEEAEVELAESGRRLRLPRDQIQRMNPPKFSKAEDMAELTCLNEASVLHNLRERYYSGLIYTYSGLFCVVINPYKQLPIYTEAIVEMYRGKKRHEVPPHVYAVTEGAYRSMLQDREDQSILCTGESGAGKTENTKKVIQYLAHVASSPKGRKEPGVPGELERQLLQANPILEAFGNAKTVKNDNSSRFGKFIRINFDVAGYIVGANIETYLLEKSRAIRQAKDECSFHIFYQLLGGAGEQLKADLLLEPCSHYRFLTNGPSSSPGQERELFQETLESLRVLGFSHEEIISMLRMVSAVLQFGNIALKRERNTDQATMPDNTAAQKLCRLLGLGVTDFSRALLTPRIKVGRDYVQKAQTKEQADFALEALAKATYERLFRWLVLRLNRALDRSPRQGASFLGILDIAGFEIFQLNSFEQLCINYTNEKLQQLFNHTMFVLEQEEYQREGIPWTFLDFGLDLQPCIDLIERPANPPGLLALLDEECWFPKATDKSFVEKVAQEQGGHPKFQRPRHLRDQADFSVLHYAGKVDYKANEWLMKNMDPLNDNVAALLHQSTDRLTAEIWKDVEGIVGLEQVSSLGDGPPGGRPRRGMFRTVGQLYKESLSRLMATLSNTNPSFVRCIVPNHEKRAGKLEPRLVLDQLRCNGVLEGIRICRQGFPNRILFQEFRQRYEILTPNAIPKGFMDGKQACEKMIQALELDPNLYRVGQSKIFFRAGVLAQLEEERDLKVTDIIVSFQAAARGYLARRAFQKRQQQQSALRVMQRNCAAYLKLRHWQWWRLFTKVKPLLQVTRQDEVLQARAQELQKVQELQQQSAREVGELQGRVAQLEEERARLAEQLRAEAELCAEAEETRGRLAARKQELELVVSELEARVGEEEECSRQMQTEKKRLQQHIQELEAHLEAEEGARQKLQLEKVTTEAKMKKFEEDLLLLEDQNSKLSKERKLLEDRLAEFSSQAAEEEEKVKSLNKLRLKYEATIADMEDRLRKEEKGRQELEKLKRRLDGESSELQEQMVEQQQRAEELRAQLGRKEEELQAALARAEDEGGARAQLLKSLREAQAALAEAQEDLESERVARTKAEKQRRDLGEELEALRGELEDTLDSTNAQQELRSKREQEVTELKKTLEEETRIHEAAVQELRQRHGQALGELAEQLEQARRGKGAWEKTRLALEAEVSELRAELSSLQTARQEGEQRRRRLELQLQEVQGRAGDGERARAEAAEKLQRAQAELENVSGALNEAESKTIRLSKELSSTEAQLHDAQELLQEETRAKLALGSRVRAMEAEAAGLREQLEEEAAARERAGRELQTAQAQLSEWRRRQEEEAGALEAGEEARRRAAREAEALTQRLAEKTETVDRLERGRRRLQQELDDATMDLEQQRQLVSTLEKKQRKFDQLLAEEKAAVLRAVEERERAEAEGREREARALSLTRALEEEQEAREELERQNRALRAELEALLSSKDDVGKSVHELERACRVAEQAANDLRAQVTELEDELTAAEDAKLRLEVTVQALKTQHERDLQGRDEAGEERRRQLAKQLRDAEVERDEERKQRTLAVAARKKLEGELEELKAQMASAGQGKEEAVKQLRKMQAQMKELWREVEETRTSREEIFSQNRESEKRLKGLEAEVLRLQEELAASDRARRQAQQDRDEMADEVANGNLSKAAILEEKRQLEGRLGQLEEELEEEQSNSELLNDRYRKLLLQEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 75 - protein, 1085 aa, synthetic construct
MVQKESQATLEERESELSSNPAASAGASLEPPAAPAPGEDNPAGAGGAAVAGAAGGARRFLCGVVEGFYGRPWVMEQRKELFRRLQKWELNTYLYAPKDDYKHRMFWREMYSVEEAEQLMTLISAAREYEIEFIYAISPGLDITFSNPKEVSTLKRKLDQVSQFGCRSFALLFDDIDHNMCAADKEVFSSFAHAQVSITNEIYQYLGEPETFLFCPTEYCGTFCYPNVSQSPYLRTVGEKLLPGIEVLWTGPKVVSKEIPVESIEEVSKIIKRAPVIWDNIHANDYDQKRLFLGPYKGRSTELIPRLKGVLTNPNCEFEANYVAIHTLATWYKSNMNGVRKDVVMTDSEDSTVSIQIKLENEGSDEDIETDVLYSPQMALKLALTEWLQEFGVPHQYSSRQVAHSGAKASVVDGTPLVAAPSLNATTVVTTVYQEPIMSQGAALSGEPTTLTKEEEKKQPDEEPMDMVVEKQEETDHKNDNQILSEIVEAKMAEELKPMDTDKESIAESKSPEMSMQEDCISDIAPMQTDEQTNKEQFVPGPNEKPLYTAEPVTLEDLQLLADLFYLPYEHGPKGAQMLREFQWLRANSSVVSVNCKGKDSEKIEEWRSRAAKFEEMCGLVMGMFTRLSNCANRTILYDMYSYVWDIKSIMSMVKSFVQWLGCRSHSSAQFLIGDQEPWAFRGGLAGEFQRLLPIDGANDLFFQPPPLTPTSKVYTIRPYFPKDEEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 76 - protein, 511 aa, synthetic construct
MMFRDQVGVLAGWFKGWNECEQTVALLSLLKRVSQTQARFLQLCLEHSLADCAELHVLEREANSPDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 77 - protein, 825 aa, synthetic construct
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 78 - protein, 1262 aa, synthetic construct
MEAKDQKKHRKKNSGPKAAKKKKRLLQDLQLGDEEDARKRNPKAFAVQSAVRMARSFHRTQDLKTKKHHIPVVDRTPLEPPPIVVVVMGPPKVGKSTLIQCLIRNFTRQKLTEIRGPVTIVSGKKRRLTIIECGCDINMMIDLAKVADLVLMLIDASFGFEMETFEFLNICQVHGFPKIMGVLTHLDSFKHNKQLKKTKKRLKHRFWTEVYPGAKLFYLSGMVHGEYQNQEIHNLGRFITVMKFRPLTWQTSHPYILADRMEDLTNPEDIRTNIKCDRKVSLYGYLRGAHLKNKSQIHMPGVGDFAVSDISFLPDPCALPEQQKKRCLNEKEKLVYAPLSGVGGVLYDKDAVYVDLGGSHVFQDEVGPTHELVQSLISTHSTIDAKMASSRVTLFSDSKPLGSEDIDNQGLMMPKEEKQMDLNTGRMRRKAIFGDEDESGDSDDEEDDEMSEDDGLENGSSDEEAEEEENAEMTDQYMAVKGIKRRKLELEEDSEMDLPAFADSDDDLERSSAEEGEAEEADESSEEEDCTAGEKGISGSKAAGEGSKAGLSPANCQSDRVNLEKSLLMKKAALPTFDSGHCTAEEVFASEDESEESSSLSAEEEDSENEEAIRKKLSKPSQVSSGQKLGPQNFIDETSDIENLLKEEEDYKEENNDSKETSGALKWKEDLSRKAAEAFLRQQQAAPNLRKLIYGTVTEDNEEEDDDTLEELGGLFRVNQPDRECKHKADSLDCSRFLVEAPHDWDLEEVMNSIRDCFVTGKWEDDKDAAKVLAEDGGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 79 - protein, 851 aa, synthetic construct
MTTAVERKYINIRKRLDQLGYRQTLTVECLPLVEKLFSDLVHTTESLRQSKLSAVKAEKESANFDFVLEPYKLENARLSRENNELYLELMKLREHSDQHVKELKTSLKKCARETADLKFLNNQYAHKLKLLEKESKAKNERIQQLQEKNLHAVVQTPGGKKRSIAFRRQRMQIDEPVPPSEVSSYPVPQPDDPYIADLLQVADNRIQELQQEVHQLQEKLAMMESGVRDYSKQIELREREIERLSVALDGGRSPDVLSLESRNKTNEKLIAHLNIQVDFLQQANKDLEKRIRELMETKETVTSEVVNLSNKNEKLCQELTEIDQLAQQLERHKEEVLETADKELGEAKKEIKRKLSEMQDLEETMAKLQLELNLCQKEKERLSDELLVKSDLETVVHQLEQEKQRLSKKVESFAVTERQLTLEVERMRLEHGIKRRDRSPSRLDTFLKGIEEERDYYKKELERLQHIIQRRSCSTSYSAREKSSIFRTPEKEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 80 - protein, 868 aa, synthetic construct
MLRRILQRTPGRVGSQGSDLDSSATPINTVDVNNESSSEGFICPQCMKSLGSADELFKHYEAVHDAGNDSGHGGESNLALKRDDVTLLRQEVQDLQASLKEEKWYSEELKKELEKYQGLQQQEAKPDGLVTDSSAELQSLEQQLEEAQTENFNIKQMKDLFEQKAAQLATEIADIKSKYDEERSLREAAEQKVTRLTEELNKEATVIQDLKTELLQRPGIEDVAVLKKELVQVQTLMDNMTLERERESEKLKDECKKLQSQYASSEATISQLRSELAKGPQEVAVYVQELQKLKSSVNELTQKNQTLTENLLKKEQDYTKLEEKHNEESVSKKNIQATLHQKDLDCQQLQSRLSASETSLHRIHVELSEKGEATQKLKEELSEVETKYQHLKAEFKQLQQQREEKEQHGLQLQSEINQLHSKLLETERQLGEAHGRLKEQRQLSSEKLMDKEQQVADLQLKLSRLEEQLKEKVTNSTELQHQLDKTKQQHQEQQALQQSTTAKLREAQEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 81 - protein, 778 aa, synthetic construct
MPRRGLILHTRTHWLLLGLALLCSLVLFMYLLECAPQTDGNASLPGVVGENYGKEYYQALLQEQEEHYQTRATSLKRQIAQLKQELQEMSEKMRSLQERRNVGANGIGYQSNKEQAPSDLLEFLHSQIDKAEVSIGAKLPSEYGVIPFESFTLMKVFQLEMGLTRHPEEKPVRKDKRDELVEVIEAGLEVINNPDEDDEQEDEEGPLGEKLIFNENDFVEGYYRTERDKGTQYELFFKKADLTEYRHVTLFRPFGPLMKVKSEMIDITRSIINIIVPLAERTEAFVQFMQNFRDVCIHQDKKIHLTVVYFGKEGLSKVKSILESVTSESNFHNYTLVSLNEEFNRGRGLNVGARAWDKGEVLMFFCDVDIYFSAEFLNSCRLNAEPGKKVFYPVVFSLYNPAIVYANQEVPPPVEQQLEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 82 - protein, 1334 aa, synthetic construct
MEENESQKCEPCLPYSADRRQMQEQGKGNLHVTSPEDAECRRTKERLSNGNSRGSVSKSSRNIPRRHTLGGPRSSKEILGMQTSEMDRKREAFLEHLKQKYPHHASAIMGHQERLRDQTRSPKLSHSPQPPSLGDPVEHLSETSADSLEAMSEGDAPTPFSRGSRTRASLPVVRSTNQTKERSLGVLYLQYGDETKQLRMPNEITSADTIRALFVSAFPQQLTMKMLESPSVAIYIKDESRNVYYELNDVRNIQDRSLLKVYNKDPAHAFNHTPKTMNGDMRMQRELVYARGDGPGAPRPGSTAHPPHAIPNSPPSTPVPHSMPPSPSRIPYGGTRSMVVPGNATIPRDRISSLPVSRPISPSPSAILERRDVKPDEDMSGKNIAMYRNEGFYADPYLYHEGRMSIASSHGGHPLDVPDHIIAYHRTAIRSASAYCNPSMQAEMHMEQSLYRQKSRKYPDSHLPTLGSKTPPASPHRVSDLRMIDMHAHYNAHGPPHTMQPDRASPSRQAFKKEPGTLVYIEKPRSAAGLSSLVDLGPPLMEKQVFAYSTATIPKDRETRERMQAMEKQIASLTGLVQSALFKGPITSYSKDASSEKMMKTTANRNHTDSAGTPHVSGGKMLSALESTVPPSQPPPVGTSAIHMSLLEMRRSVAELRLQLQQMRQLQLQNQELLRAMMKKAELEISGKVMETMKRLEDPVQRQRVLVEQERQKYLHEEEKIVKKLCELEDFVEDLKKDSTAASRLVTLKDVEDGAFLLRQVGEAVATLKDVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 83 - protein, 1256 aa, synthetic construct
MSSECDGGSKAVMNGLAPGSNGQDKDMDLTKICTGKGAVTLRASSSYRETPSSSPASPQETRQHESKPDEWRLSSSADANGNAQPSSLAAKGYRSVHPNLPSDKSQDSSPLLNEVSSSLIGTDSQAFPSVSKPSSAYPSTTIVNPTIVLLQHNREQQKRLSSLSDPVSERRVGEQDSAPTQEKPTSPGKAIEKRAKDDSRRVVKSTQDLSDVSMDEVGIPLRNTERSKDWYKTMFKQIHKLNRDDDSDLYSPRYSFSEDTKSPLSVPRSKSEMSYIDGEKVVKRSATLPLPARSSSLKSSSERNDWEPPDKKVDTRKYRAEPKSIYEYQPGKSSVLTNEKMSSAISPTPEISSETPGYIYSSNFHAVKRESDGAPGDLTSLENERQIYKSVLEGGDIPLQGLSGLKRPSSSASTKDSESPRHFIPADYLESTEEFIRRRHDDKEMRPARAKFDFKAQTLKELPLQKGDIVYIYKQIDQNWYEGEHHGRVGIFPRTYIELLPPAEKAQPKKLTPVQVLEYGEAIAKFNFNGDTQVEMSFRKGERITLLRQVDENWYEGRIPGTSRQGIFPITYVDVIKRPLVKNPVDYMDLPFSSSPSRSATASPQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 84 - protein, 1108 aa, synthetic construct
MSSECDGGSKAVMNGLAPGSNGQDKDMDLTKICTGKGAVTLRASSSYRETPSSSPASPQETRQHESKPDEWRLSSSADANGNAQPSSLAAKGYRSVHPNLPSDKSQDSSPLLNEVSSSLIGTDSQAFPSVSKPSSAYPSTTIVNPTIVLLQHNREQQKRLSSLSDPVSERRVGEQDSAPTQEKPTSPGKAIEKRAKDDSRRVVKSTQDLSDVSMDEVGIPLRNTERSKDWYKTMFKQIHKLNRDDDSDLYSPRYSFSEDTKSPLSVPRSKSEMSYIDGEKVVKRSATLPLPARSSSLKSSSERNDWEPPDKKVDTRKYRAEPKSIYEYQPGKSSVLTNEKMSSAISPTPEISSETPGYIYSSNFHAVKRESDGAPGDLTSLENERQIYKSVLEGGDIPLQGLSGLKRPSSSASTKDSESPRHFIPADYLESTEEFIRRRHDDKEMRPARAKFDFKAQTLKELPLQKGDIVYIYKQIDQNWYEGEHHGRVGIFPRTYIELLPPAEKAQPKKLTPVQVLEYGEAIAKFNFNGDTQVEMSFRKGERITLLRQVDENWYEGRIPGTSRQGIFPITYVDVIKRPLVKNPVDYMDLPFSSSPSRSATASPQQPQAQQRRVTPDRSQTSQDLFSYQALYSYIPQNDDELELRDGDIVDVMEKCDDGWFVDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 85 - protein, 1375 aa, synthetic construct
MSAAKENPCRKFQANIFNKSKCQNCFKPRESHLLNDEDLTQAKPIYGGWLLLAPDGTDFDNPVHRSRKWQRRFFILYEHGLLRYALDEMPTTLPQGTINMNQCTDVVDGEGRTGQKFSLCILTPEKEHFIRAETKEIVSGWLEMLMVYPRTNKQNQKKKRKVEPPTPQEPGPAKVAVTSSSSSSSSSSSIPSAEKVPTTKSTLWQEEMRTKDQPDGSSLSPAQSPSQSQPPAASSLREPGLESKEEESAMSSDRMDCGRKVRVESGYFSLEKTKQDLKAEEQQLPPPLSPPSPSTPNHRRSQVIEKFEALDIEKAEHMETNAVGPSPSSDTRQGRSEKRAFPRKRDFTNEAPPAPLPDASASPLSPHRRAKSLDRRSTEPSVTPDLLNFKKGWLTKQYEDGQWKKHWFVLADQSLRYYRDSVAEEAADLDGEIDLSACYDVTEYPVQRNYGFQIHTKEGEFTLSAMTSGIRRNWIQTIMKHVHPTTAPDVTSSLPEEKNKSSCSFETCPRPTEKQEAELGEPDPEQKRSRARERRREGRSKTFDWAEFRPIQQALAQERVGGVGPADTHEPLRPEAEPGELERERARRREERRKRFGMLDATDGPGTEDAALRMEVDRSPGLPMSDLKTHNVHVEIEQRWHQVETTPLREEKQVPIAPVHLSSEDGGDRLSTHELTSLLEKELEQSQKEASDLLEQNRLLQDQLRVALGREQSAREGYVLQATCERGFAAMEETHQKKIEDLQRQHQRELEKLREEKDRLLAEETAATISAIEAMKNAHREEMERELEKSQRSQISSVNSDVEALRRQYLEELQSVQRELEVLSEQYSQKCLENAHLAQALEAERQALRQCQRENQELNAHNQELNNRLAAEITRLRTLLTGDGGGEATGSPLAQGKDAYELEVLLRVKESEIQYLKQEISSLKDELQTALRDKKYASDKYKDIYTELSIAKAKADCDISRLKEQLKAATEALGEKSPDSATVSGYDIMKSKSNPDFLKKDRSCVTRQLRNIRSKEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 86 - protein, 639 aa, synthetic construct
MNGQLDLSGKLIIKAQLGEDIRRIPIHNEDITYDELVLMMQRVFRGKLLSNDEVTIKYKDEDGDLITIFDSSDLSFAIQCSRILKLTLFVNGQPRPLESSQVKYLRRELIELRNKVNRLLDSLEPPGEPGPSTNIPENDTVDGREEKSASDSSGKQSTQVMAASMSAFDPLKNQDEINKNVMSAFGLTDDQVSDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 87 - protein, 1244 aa, synthetic construct
MKKASRSVGSVPKVSAISKTQTAEKIKPENSSSASTGGKLVKPGTAASLSKTKSSDDLLAGMAGGVTVTNGVKGKKSTCPSAAPSASAPAMTTVENKSKISTGTASSTKRSTSTGNKESSSTRERLRERTRLNQSKKLPSAGQGANDMALAKRSRSRTATECDVRMSKSKSDNQISDRAALEAKVKDLLTLAKTKDVEILHLRNELRDMRAQLGINEDHSEGDEKSEKETIMAHQPTDVESTLLQLQEQNTAIREELNQLKNENRMLKDRLNALGFSLEQRLDNSEKLFGYQSLSPEITPGNQSDGGGTLTSSVEGSAPGSVEDLLSQDENTLMDHQHSNSMDNLDSECSEVYQPLTSSDDALDAPSSSESEGIPSIERSRKGSSGNASEVSVACLTERIHQMEENQHSTSEELQATLQELADLQQITQELNSENERLGEEKVILMESLCQQSDKLEHFSRQIEYFRSLLDEHHISYVIDEDVKSGRYMELEQRYMDLAENARFEREQLLGVQQHLSNTLKMAEQDNKEAQEMIGALKERSHHMERIIESEQKGKAALAATLEEYKATVASDQIEMNRLKAQLENEKQKVAELYSIHNSGDKSDIQDLLESVRLDKEKAETLASSLQEDLAHTRNDANRLQDAIAKVEDEYRAFQEEAKKQIEDLNMTLEKLRSDLDEKETERSDMKETIFELEDEVEQHRAVKLHDNLIISDLENTVKKLQDQKHDMEREIKTLHRRLREESAEWRQFQADLQTAVVIANDIKSEAQEEIGDLKRRLHEAQEKNEKLTKELEEIKSRKQEEERGRVYNYMNAVERDLAALRQGMGLSRRSSTSSEPTPTVKTLIKSFDSASQVPNPAAAAIPRTPLSPSPMKTPPAAAVSPMQEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 88 - protein, 499 aa, synthetic construct
MVSWMISRAVVLVFGMLYPAYYSYKAVKTKNVKEYVRWMMYWIVFALYTVIETVADQTVAWFPLYYELKIAFVIWLLSPYTKGASLIYRKFLHPLLSSKEREIDDYIVQAKERGYETMVNFGRQGLNLAATAAVTAAVKEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 89 - protein, 1244 aa, synthetic construct
MDIYDTQTLGVVVFGGFMVVSAIGIFLVSTFSMKETSYEEALANQRKEMAKTHHQKVEKKKKEKTVEKKGKTKKKEEKPNGKIPDHDPAPNVTVLLREPVRAPAVAVAPTPVQPPIIVAPVATVPAMPQEKLASSPKDKKKKEKKVAKVEPAVSSVVNSIQVLTSKAAILETAPKEGRNTDVAQSPEAPKQEAPAKKKSGSKKKGPPDADGPLYLPYKTLVSTVGSMVFNEGEAQRLIEILSEKAGIIQDTWHKATQKGDPVAILKRQLEEKEKLLATEQEDAAVAKSKLRELNKEMAAEKAKAAAGEAKVKKQLVAREQEITAVQARMQASYREHVKEVQQLQGKIRTLQEQLENGPNTQLARLQQENSILRDALNQATSQVESKQNAELAKLRQELSKVSKELVEKSEAVRQDEQQRKALEAKAAAFEKQVLQLQASHRESEEALQKRLDEVSRELCHTQSSHASLRADAEKAQEQQQQMAELHSKLQSSEAEVRSKCEELSGLHGQLQEARAENSQLTERIRSIEALLEAGQARDAQDVQASQAEADQQQTRLKELESQVSGLEKEAIELREAVEQQKVKNNDLREKNWKAMEALATAEQACKEKLLSLTQAKEESEKQLCLIEAQTMEALLALLPELSVLAQQNYTEWLQDLKEKGPTLLKHPPAPAEPSSDLASKLREAEETQSTLQAECDQYRSILAETEGMLRDLQKSVEEEEQVWRAKVGAAEEELQKSRVTVKHLEEIVEKLKGELESSDQVREHTSHLEAELEKHMAAASAECQNYAKEVAGLRQLLLESQSQLDAAKSEAQKQSDELALVRQQLSEMKSHVEDGDIAGAPASSPEAPPAEQDPVQLKTQLEWTEAILEDEQTQRQKLTAEFEEEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 90 - protein, 744 aa, synthetic construct
MSETPAQCSIKQERISYTPPESPVPSYASSTPLHVPVPRALRMEEDSIRLPAHLRLQPIYWSRDDVAQWLKWAENEFSLRPIDSNTFEMNGKALLLLTKEDFRYRSPHSGDVLYELLQHILKQRKPRILFSPFFHPGNSIHTQPEVILHQNHEEDNCVQRTPRPSVDNVHHNPPTIELLHRSRSPITTNHRPSPDPEQRPLRSPLDNMIRRLSPAERAQGPRPHQENNHQESYPLSVSPMENNHCPASSESHPKPSSPRQESTRVIQLMPSPIMHPLILNPRHSVDFKQSRLSEDGLHREGKPINLSHREDLAYMNHIMVSVSPPEEHAMPIGRIADCRLLWDYVYQLLSDSRYENFIRWEDKESKIFRIVDPNGLARLWGNHKEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 91 - protein, 1104 aa, synthetic construct
MCESYSRSLLRVSVAQICQALGWDSVQLSACHLLTDVLQRYLQQLGRGCHRYSELYGRTDPILDDVGEAFQLMGVSLHELEDYIHNIEPVTFPHQIPSFPVSKNNVLQFPQPGSKDAEERKEYIPDYLPPIVSSQEEEEEEQVPTDGGTSAEAMQVPLEEDDELEEEEIINDENFLGKRPLDSPEAEELPAMKRPRLLSTKGDTLDVVLLEAREPLSSINTQKIPPMLSPVHVQDSTDLAPPSPEPPMLAPVAKSQMPTAKPLETKSFTPKTKTKTSSPGQKTKSPKTAQSPAMVGSPIRSPKTVSKEKKSPGRSKSPKSPKSPKVTTHIPQTPVRPETPNRTPSATLSEKISKETIQVKQIQTPPDAGKLNSENQPKKAVVADKTIEASIDAVIARACAEREPDPFEFSSGSESEGDIFTSPKRISGPECTTPKASTSANNFTKSGSTPLPLSGGTSSSDNSWTMDASIDEVVRKAKLGTPSNMPPNFPYISSPSVSPPTPEPLHKVYEEKTKLPSSVEVKKKLKKELKTKMKKKEKQRDREREKDKNKDKSKEKDKVKEKEKDKETGRETKYPWKEFLKEEEADPYKFKIKEFEDVDPKVKLKDGLVRKEKEKHKDKKKDREKGKKDKDKREKEKVKDKGREDKMKAPAPPLVLPPKELALPLFSPATASRVPAMLPSLLPVLPEKLFEEKEKVKEKEKKKDKKEKKKKKEKEKEKKEKEREKEKREREKREKEKEKHKHEKEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 92 - protein, 1969 aa, synthetic construct
MATGGGPFEDGMNDQDLPNWSNENVDDRLNNMDWGAQQKKANRSSEKNKKKFGVESDKRVTNDISPESSPGVGRRRTKTPHTFPHSRYMSQMSVPEQAELEKLKQRINFSDLDQRSIGSDSQGRATAANNKRQLSENRKPFNFLPMQINTNKSKDASTNPPNRETIGSAQCKELFASALSNDLLQNCQVSEEDGRGEPAMESSQIVSRLVQIRDYITKASSMREDLVEKNERSANVERLTHLIDHLKEQEKSYMKFLKKILARDPQQEPMEEIENLKKQHDLLKRMLQQQEQLRALQGRQAALLALQHKAEQAIAVMDDSVVAETAGSLSGVSITSELNEELNDLIQRFHNQLRDSQPPAVPDNRRQAESLSLTREVSQSRKPSASERLPDEKVELFSKMRVLQEKKQKMDKLLGELHTLRDQHLNNSSSSPQRSVDQRSTSAPSASVGLAPVVNGESNSLTSSVPYPTASLVSQNESENEGHLNPSEKLQKLNEVRKRLNELRELVHYYEQTSDMMTDAVNENRKDEETEESEYDSEHENSEPVTNIRNPQVASTWNEVNSHSNAQCVSNNRDGRTVNSNCEINNRSAANIRALNMPPSLDCRYNREGEQEIHVAQGEDDEEEEEEAEEEGVSGASLSSHRSSLVDEHPEDAEFEQKINRLMAAKQKLRQLQDLVAMVQDDDAAQGVISASASNLDDFYPAEEDTKQNSNNTRGNANKTQKDTGVNEKAREKFYEAKLQQQQRELKQLQEERKKLIDIQEKIQALQTACPDLQLSAASVGNCPTKKYMPAVTSTPTVNQHETSTSKSVFEPEDSSIVDNELWSEMRRHEMLREELRQRRKQLEALMAEHQRRQGLAETASPVAVSLRSDGSENLCTPQQSRTEKTMATWGGSTQCALDEEGDEDGYLSEGIVRTDEEEEEEQDASSNDNFSVCPSNSVNHNSYNGKETKNRWKNNCPFSADENYRPLAKTRQQNISMQRQENLRWVSELSYVEEKEQWQEQINQLKKQLDFSVSICQTLMQDQQTLSCLLQTLLTGPYSVMPSNVASPQVHFIMHQLNQCYTQLTWQQNNVQRLKQMLNELMRQQNQHPEKPGGKERGSSASHPPSPSLFCPFSFPTQPVNLFNIPGFTNFSSFAPGMNFSPLFPSNFGDFSQNISTPSEQQQPLAQNSSGKTEYMAFPKPFESSSSIGAEKPRNKKLPEEEVESSRTPWLYEQEGEVEKPFIKTGFSVSVEKSTSSNRKNQLDTNGRRRQFDEESLESFSSMPDPVDPTTVTKTFKTRKASAQASLASKDKTPKSKSKKRNSTQLKSRVKNIRYESASMSSTCEPCKSRNRHSAQTEEPVQAKVFSRKNHEQLEKIIKCNRSTEISSETGSDFSMFEALRDTIYSEVATLISQNESRPHFLIELFHELQLLNTDYLRQRALYALQDIVSRHISESHEKGENVKSVNSGTWIASNSELTPSESLATTDDETFEKNFERETHKISEQNDADNASVLSVSSNFEPFATDDLGNTVIHLDQALARMREYERMKTEAESNSNMRCTCRIIEDGDGAGAGTTVNNLEETPVIENRSSQQPVSEVSTIPCPRIDTQQLDRQIKAIMKEVIPFLKEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 93 - protein, 1969 aa, synthetic construct; identical to SEQ ID NO: 92

SEQ ID NO: 94 - protein, 702 aa, synthetic construct
MSRDPGSGGWEEAPRAAAALCTLYHEAGQRLRRLQDQLAARDALIARLRARLAALEGDAAPSLVDALLEQVARFREQLRRQEGGAAEAQMRQEIERLTERLEEKEREMQQLLSQPQHEREKEVVLLRRSMAEGERARAASDVLCRSLANETHQLRRTLTATAHMCQHLAKCLDERQHAQRNVGERSPDQSEHTDGHTSVQSVIEKLQEENRLLKQKVTHVEDLNAKWQRYNASRDEYVRGLHAQLRGLQIPHEPELMRKEISRLNRQLEEKINDCAEVKQELAASRTARDAALERVQMLEQQILAYKDDFMSERADRERAQSRIQELEEKVASLLHQVSWRQEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 95 - protein, 762 aa, synthetic construct
MDHLNEATQGKEHSEMSNNVSDPKGPPAKIARLEQNGSPLGRGRLGSTGAKMQGVPLKHSGHLMKTNLRKGTMLPVFCVVEHYENAIEYDCKEEHAEFVLVRKDMLFNQLIEMALLSLGYSHSSAAQAKGLIQVGKWNPVPLSYVTDAPDATVADMLQDVYHVVTLKIQLHSCPKLEDLPPEQWSHTTVRNALKDLLKDMNQSSLAKECPLSQSMISSIVNSTYYANVSAAKCQEFGRWYKHFKKTKDMMVEMDSLSELSQQGANHVNFGQQPVPGNTAEQPPSPAQLSHGSQPSVRTPLPNLHPGLVSTPISPQLVNQQLVMAQLLNQQYAVNRLLAQQSLNQQYLNHPPPVSRSMNKPLEQQVSTNTEVSSEIYQWVRDELKRAGISQAVFARVAFNRTQEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 96 - protein, 1170 aa, synthetic construct
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILCFPGCLTIQIRTVLCIFIVVLIYSVAQGCVVGCLPWAWSSKPNSSLVVLSSGGQRTALPTLPCESTHHALLCCLVGTLPLAIFFRVSSLPKMILLSGLTTSYILVLELSGYTRTGGGAVSGRSYEPIVAILLFSCALALHARQVDIRLRLDYLWAAQAEEEREDMEKVKLDNRRILFNLLPAHVAQHFLMSNPRNMDLYYQSYSQVGVMFASIPNFNDFYIELDGNNMGVECLRLLNEIIADFDELMEKDFYKDIEKIKTIGSTYMAAVGLAPTSGTKAKKSISSHLSTLADFAIEMFDVLDEINYQSYNDFVLRVGINVGPVVAGVIGARRPQYDIWGNTVNVASRMDSTGVQGRIQVTEEVHRLLRRCPYHFVCRGKVSVKGKGEMLTYFLEGRTDGNGSQIRSLGLDRKMCPFGRAGLQGRRPPVCPMPGVSVRAGLPPHSPGQYLPSAAAGKEA

SEQ ID NO: 97 - protein, 1286 aa, synthetic construct
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEQVSFKDVCVDFTQEEWYLLDPAQKILYRDVILENYSNLVSVGYCITKPEVIFKIEQGEEPWILEKGFPSQCHPERKWKVDDVLESSQENEDDHFWELLFHNNKTVSVENGDRGSKTFNLGTDPVSLRNYPYKICDSCEMNLKNISGLIISKKNCSRKKPDEFNVCEKLLLDIRHEKIPIGEKSYKYDQKRNAINYHQDLSQPSFGQSFEYSKNGQGFHDEAAFFTNKRSQIGETVCKYNECGRTFIESLKLNISQRPHLEMEPYGCSICGKSFCMNLRFGHQRALTKDNPYEYNEYGEIFCDNSAFIIHQGAYTRKILREYKVSDKTWEKSALLKHQIVHMGGKSYDYNENGSNFSKKSHLTQLRRAHTGEKTFECGECGKTFWEKSNLTQHQRTHTGEKPYECTECGKAFCQKPHLTNHQRTHTGEKPYECKQCGKTFCVKSNLTEHQRTHTGEKPYECNACGKSFCHRSALTVHQRTHTGEKPFICNECGKSFCVKSNLIVHQRTHTGEKPYKCNECGKTFCEKSALTKHQRTHTGEKPYECNACGKTFSQRSVLTKHQRIHTRVKALSTS

SEQ ID NO: 98 - protein, 1212 aa, synthetic construct
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKGNLGRVDQVSEFEYDLFIRPDTCNPRFRVWFNFTVENVKESQRVIFNIVNFSKTKSLYRDGMAPMVKSTSRPKWQRLPPKNVYYYRCPDHRKNYVMSFAFCFDREEDIYQFAYCYPYTYTRFQHYLDSLQKRNMDYFFREQLGQSVQQRKLDLLTITSPDNLREGAEQKVVFITGRVHPGETPSSFVCQGIIDFLVSQHPIACVLREYLVFKIAPMLNPDGVYLGNYRCSLMGFDLNRHWLDPSPWVHPTLHGVKQLIVQMYNDPKTSLEFYIDIHAHSTMMNGFMYGNIFEDEERFQRQAIFPKLLCQNAEDFSYSSTSFNRDAVKAGTGRRFLGGLLDHTSYCYTLEVSFYSYIISGTTAAVPYTEEAYMKLGRNVARTFLDYYRLNPVVEKVAIPMPRLRNKEIEVQRRKEKSPPYKHPLLRGPASNYPNSKGDKKSSVNHKDPSTPF

SEQ ID NO: 99 - protein, 652 aa, synthetic construct
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQGVLGKCRYVYYGKNSEGNRFIRDDQL

SEQ ID NO: 100 - protein, 897 aa, synthetic construct
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILAPAYIRECARLHYLGSRTQASKDDLDLIASCHTKSFQLAKSQKRNRVDSCPHQEETQHHTEEALRELFQHVHDMPESAKKKQLIRQFNKQSLTQTPGREPSTSQVQKRARSRSFSGLIKRKVLGNQMMSEKKKKNPTPESVAIGELKGTSKENRNLLFSGSPAVTMTPTRLKWSEGKKEGKKGFL

SEQ ID NO: 101 - protein, 886 aa, synthetic construct
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILVQIRPWNLSDSDFVMDGSQPLDPRKTIFVGGVPRPLRAVELAMIMDRLYGGVCYAGIDTDPELKYPKGAGRVAFSNQQSYIAAISARFVQLQHNDIDKRVEVKPYVLDDQMCDECQGTRCGGKFAPFFCANVTCLQYYCEYCWASIHSRAGREFHKPLVKEGGDRPRHVPFRWS

SEQ ID NO: 102 - protein, 1613 aa, synthetic construct
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLPIPSSSVLPSAPRDVVPVLVSSRFVRLSWRPPAEAKGNIQTFTVFFSREGDNRERALNTTQPGSLQLTVGNLKPEAMYTFRVVAYNEWGPGESSQPIKVATQPELQVPGPVENLQAVSTSPTSILITWEPPAYANGPVQGYRLFCTEVSTGKEQNIEVDGLSYKLEGLKKFTEYSLRFLAYNRYGPGVSTDDITVVTLSDVPSAPPQNVSLEVVNSRSIKVSWLPPPSGTQNGFITGYKIRHRKTTRRGEMETLEPNNLWYLFTGLEKGSQYSFQVSAMTVNGTGPPSNWYTAETPENDLDESQVPDQPSSLHVRPQTNCIIMSWTPPLNPNIVVRGYIIGYGVGSPYAETVRVDSKQRYYSIERLESSSHYVISLKAFNNAGEGVPLYESATTRSITDPTDPVDYYPLLDDFPTSVPDLSTPMLPPVGVQAVALTHDAVRVSWADNSVPKNQKTSEVRLYTVRWRTSFSASAKYKSEDTTSLSYTATGLKPNTMYEFSVMVTKNRRSSTWSMTAHATTYEAAPTSAPKDLTVITREGKPRAVIVSWQPPLEANGKITAYILFYTLDKNIPIDDWIMETISGDRLTHQIMDLNLDTMYYFRIQARNSKGVGPLSDPILFRTLKVEHPDKMANDQGRHGDGGYWPVDTNLIDRSTLNEPPIGQMHPPHGSVTPQKNSNLLVIIVVTVGVITVLVVVIVAVICTRRSSAQQRKKRATHSAGKRKGSQKDLRPPDLWIHHEEMEMKNIEKPSGTDPAGRDSPIQSCQDLTPVSHSQSETQLGSKSTSHSGQDTEEAGSSMSTLERSLAARRAPRAKLMIPMDAQSNNPAVVSAIPVPTLESAQYPGILPSPTCGYPHPQFTLRPVPFPTLSVDRGFGAGRSQSVSEGPTTQQPPMLPPSQPEHSSSEEAPSRTIPTACVRPTHPLRSFANPLLPPPMSAIEPKVPYTPLLSQPGPTLPKTHVKTASLGLAGKARSPLLPVSVPTAPEVSEESHKPTEDSANVYEQDDLSEQMASLEGLMKQLNAITGSAF

SEQ ID NO: 103 - protein, 1077 aa, synthetic construct
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILNHVNPAMDFTQTPPGMLALDNMLYFAKHHQDAYIRIVLENSSREDKHECPFGRSSIELTKMLCEILKVGELPSETCNDFHPMFFTHDRSFEEFFCICIQLLNKTWKEMRATSEDFNKVMQVVKEQVMRALTTKPSSLDQFKSKLQNLSYTEILKIRQSERMNQEDFQSRPILELKEKIQPEILELIKQQRLNRLVEGTCFRKLNARRRQDKFWYCRLSPNHKVLHYGDLEESPQGEVPHDSLQDKLPVADIKAVVTGKDCPHMKEKGALKQNKEVLELAFSILYDSNCQLNFIAPDKHEYCIWTDGLNALLGKDMMSDLTRNDLDTLLSMEIKLRLLDLENIQIPDAPPPIPKEPSNYDFVYDCN

SEQ ID NO: 104 - protein, 1873 aa, synthetic construct
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQDFVSCIENYRRRGQELYASLYKDHVQRRKCGNIKAANAWARIQEQLFGELGLWSQGEETKPCSPWELDWREGPARMRKRIKRLSPLEALSSGRHKESQDKNDHISQTNAENQDELTLREAEGEPDEVGVDCTQLTFFPALHESLHSEDFLELCRERQVILQELLDKEKVTQKFSLVIVQGHLVSEGVLLFGHQHFYICENFTLSPTGDVYCTRHCLSNISDPFIFNLCSKDRSTDHYSCQCHSYADMRELRQARFLLQDIALEIFFHNGYSKFLVFYNNDRSKAFKSFCSFQPSLKGKATSEDTLSLRRYPGSDRIMLQKWQKRDISNFEYLMYLNTAAGRTCNDYMQYPVFPWVLADYTSETLNLANPKIFRDLSKPMGAQTKERKLKFIQRFKEVEKTEGDMTVQCHYYTHYSSAIIVASYLVRMPPFTQAFCALQGGSFDVADRMFHSVKSTWESASRENMSDVRELTPEFFYLPEFLTNCNGVEFGCMQDGTVLGDVQLPPWADGDPRKFISLHRKALESDFVSANLHHWIDLIFGYKQQGPAAVDAVNIFHPYFYGDRMDLSSITDPLIKSTILGFVSNFGQVPKQLFTKPHPARTAAGKPLPGKDVSTPVSLPGHPQPFFYSLQSLRPSQVTVKDMYLFSLGSESPKGAIGHIVSTEKTILAVERNKVLLPPLWNRTFSWGFDDFSCCLGSYGSDKVLMTFENLAAWGRCLCAVCPSPTTIVTSGTSTVVCVWELSMTKGRPRGLRLRQALYGHTQAVTCLAASVTFSLLVSGSQDCTCILWDLDHLTHVTRLPAHREGISAITISDVSGTIVSCAGAHLSLWNVNGQPLASITTAWGPEGAITCCCLMEGPAWDTSQIIITGSQDGMVRVWKTEDVKMSVPGRPAGEEPPAQPPSPRGHKWEKNLALSRELDVSIALTGKPSKTSPAVTALAVSRNHTKLLVGDERGRIFCWSADG

SEQ ID NO: 105 - protein, 1765 aa, synthetic construct
MKKIFSKKGESPLGSFARRQRSSAGGGGEPGEGAYSQPGYHVRDRDLGKIHKAASAGNVAKVQQILLLRKNGLNDRDKMNRTALHLACANGHPEVVTLLVDRKCQLNVCDNENRTALMKAVQCQEEKCATILLEHGADPNLADVHGNTALHYAVYNEDISVATKLLLYDANIEAKNKDDLTPLLLAVSGKKQQMVEFLIKKKANVNAVDKLESSHQLISEYKEERIPKHSSQNSNSVDESSEDSLSRLSGKPGVDDSWPTSDDEDLNFDTKNVPKPSLAKLMTASQQSRKNLEATYGTVRTGNRTLFEDRDSDSQDEVVVESLPTTSIKVQCFSHPTYQSPDLLPKPSHKSLANPGLMKEEPTKPGIAKKENGIDIIESAPLEQTNNDNLTYVDEVHKNNRSDMMSALGLGQEEDIESPWDSESISENFPQKYVDPLAGAADGKEKNIGNEQAEDVFYIPSCMSGSRNFKMAKLEDTRNVGMPVAHMESPERYLHLKPTIEMKDSVPNKAGGMKDVQTSKAAEHDLEVASEEEQEREGSENNQPQVEEERKKHRNNEMEVSANIHDGATDDAEDDDDDDGLIQKRKSGETDHQQFPRKENKEYASSGPALQMKEVKSTEKEKRTSKESVNSPVFGKASLLTGGLLQVDDDSSLSEIDEDEGRPTKKTSNEKNKVKNQIQSMDDVDDLTQSSETASEDCELPHSSYKNFMLLIEQLGMECKDSVSLLKIQDAALSCERLLELKKNHCELLTVKIKKMEDKVNVLQRELSETKEIKSQLEHQKVEWERELCSLRFSLNQEEEKRRNADTLYEKIREQLRRKEEQYRKEVEVKQQLELSLQTLEMELRTVKSNLNQVVQERNDAQRQLSREQNARMLQDGILTNHLSKQKEIEMAQKKMNSENSHSHEEEKDLSHKNSMLQEEIAMLRLEIDTIKNQNQEKEKKCFEDLKIVKEKNEDLQKTIKQNEETLTQTISQYNGRLSVLTAENAMLNSKLENEKQSKERLEAEVESYHSRLAAAIHDRDQSETSKRELELAFQRARDECSRLQDKMNFDVSNLKDNNEILSQQLFKTESKLNSLEIEFHHTRDALREKTLGLERVQKDLSQTQCQMKEMEQKYQNEQVKVNKYIGKQESVEERLSQLQSENMLLRQQLDDAHNKADNKEKTVINIQDQFHAIVQKLQAESEKQSLLLEERNKELISECNHLKERQYQYENEKAEREVVVRQLQQELADTLKKQSMSEASLEVTSRYRINLEDETQDLKKKLGQIRNQLQEAQDRHTEAVRCAEKMQDHKQKLEKDNAKLKVTVKKQMDKIEELQKNLLNANLSEDEKEQLKKLMELKQSLECNLDQEMKKNVELEREITGFKNLLKMTRKKLNEYENGEFSFHGDLKTSQFEMDIQINKLKHKEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 106 - protein, 1432 aa, synthetic construct
MSMLKPSGLKAPTKILKPGSTALKTPTAVVAPVEKTISSEKASSTPSSETQEEFVDDFRVGERVWVNGNKPGFIQFLGETQFAPGQWAGIVLDEPIGKNDGSVAGVRYFQCEPLKGIFTRPSKLTRKVQAEDEANGLQTTPASRATSPLCTSTASMVSSSPSTPSNIPQKPSQPAAKEPSATPPISNLTKTASESISNLSEAGSIKKGERELKIGDRVLVGGTKAGVVRFLGETDFAKGEWCGVELDEPLGKNDGAVAGTRYFQCQPKYGLFAPVHKVTKIGFPSTTPAKAKANAVRRVMATTSASLKRSPSASSLSSMSSVASSVSSRPSRTGLLTETSSRYARKISGTTALQEALKEKQQHIEQLLAERDLERAEVAKATSHVGEIEQELALARDGHDQHVLELEAKMDQLRTMVEAADREKVELLNQLEEEKRKVEDLQFRVEEESITKGDLETQTKLEHARIKELEQSLLFEKTKADKLQRELEDTRVATVSEKSRIMELEKDLALRVQEVAELRRRLESNKPAGDVDMSLSLLQEISSLQEKLEVTRTDHQREITSLKEHFGAREETHQKEIKALYTATEKLSKENESLKSKLEHANKENSDVIALWKSKLETAIASHQQAMEELKVSFSKGLGTETAEFAELKTQIEKMRLDYQHEIENLQNQQDSERAAHAKEMEALRAKLMKVIKEKENSLEAIRSKLDKAEDQHLVEMEDTLNKLQEAEIKVKELEVLQAKCNEQTKVIDNFTSQLKATEEKLLDLDALRKASSEGKSEMKKLRQQLEAAEKQIKHLEIEKNAESSKASSITRELQGRELKLTNLQENLSEVSQVKETLEKELQILKEKFAEASEEAVSVQRSMQETVNKLHQKEEQFNMLSSDLEKLRENLADMEAKFREKDEREEQLIKAKEKLENDIAEIMKMSGDNSSQLTKMNDELRLKERDVEELQLKLTKANENASFLQKSIEDMTVKAEQSQQEAAKKHEEEKKELERKLSDLEKKMETSHNQCQELKARYERATSETKTKHEEILQNLQKTLLDTEDKLKGAREENSGLLQELEELRKQADKAKEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 107 - protein, 1154 aa, synthetic construct
MEPQRRELLAQCQQSLAQAMTEVEAVLGLLEAAGALSPGERRQLDEEAGGAKAELLLKLLLAKERDHFQDLRAALEKTQPHLLPILYLNGVVGPPQPAEGAGSTYSVLSTMPSDSESSSSLSSVGTTGKAPSPPPLLTDQQVNEKVENLSIQLRLMTRERNELRKRLAFATHGTAFDKRPYHRLNPDYERLKIQCVRAMSDLQSLQNQHTNALKRCEEVAKETDFYHTLHSRLLSDQTRLKDDVDMLRRENGQLLRERNLLQQSWEDMKRLHEEDQKEIGDLRAQQQQVLKHNGSSEILNKLYDTAMDKLEVVKKDYDALRKRYSEKVAIHNADLSRLEQLGEENQRLLKQTEMLTQQRDTAIQLQHQCALSLRRFEAIHHELNKATAQNKDLQWEMELLQSELTELRTTQVKTAKESEKYREERDAVYSEYKLIMSERDQVISELDKLQTEVELAESKLKSSTSEKKAANEEMEALRQIKDTVTMDAGRANKEVEILRKQCKALCQELKEALQEADVAKCRRDWAFQERDKIVAERDSIRTLCDNLRRERDRAVSELAEALRSLDDTRKQKNDVSRELKELKEQMESQLEKEARFRQLMAHSSHDSAIDTDSMEWETEVVEFERETEDIDLKALGFDMAEGVNEPCFPGDCGIFVTKVDKGSIADGRLRVNDWLLRINDVDLINKDKKQAIKALLNGEGAINMVVRRRKSLGGKVVTPLHINLSGQKDSGISLENGVYAAAVLPGSPAAKEGSLAVGDRIVAINGIALDNKSLNECESLLRSCQDSLTLSLLKEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 108 - protein, 2286 aa, synthetic construct
MEPQRRELLAQCQQSLAQAMTEVEAVLGLLEAAGALSPGERRQLDEEAGGAKAELLLKLLLAKERDHFQDLRAALEKTQPHLLPILYLNGVVGPPQPAEGAGSTYSVLSTMPSDSESSSSLSSVGTTGKAPSPPPLLTDQQVNEKVENLSIQLRLMTRERNELRKRLAFATHGTAFDKRPYHRLNPDYERLKIQCVRAMSDLQSLQNQHTNALKRCEEVAKETDFYHTLHSRLLSDQTRLKDDVDMLRRENGQLLRERNLLQQSWEDMKRLHEEDQKEIGDLRAQQQQVLKHNGSSEILNKLYDTAMDKLEVVKKDYDALRKRYSEKVAIHNADLSRLEQLGEENQRLLKQTEMLTQQRDTAIQLQHQCALSLRRFEAIHHELNKATAQNKDLQWEMELLQSELTELRTTQVKTAKESEKYREERDAVYSEYKLIMSERDQVISELDKLQTEVELAESKLKSSTSEKKAANEEMEALRQIKDTVTMDAGRANKEVEILRKQCKALCQELKEALQEADVAKCRRDWAFQERDKIVAERDSIRTLCDNLRRERDRAVSELAEALRSLDDTRKQKNDVSRELKELKEQMESQLEKEARFRQLMAHSSHDSAIDTDSMEWETEVVEFERETEDIDLKALGFDMAEGVNEPCFPGDCGIFVTKVDKGSIADGRLRVNDWLLRINDVDLINKDKKQAIKALLNGEGAINMVVRRRKSLGGKVVTPLHINLSGQKDSGISLENGVYAAAVLPGSPAAKEGSLAVGDRIVAINGIALDNKSLNECESLLRSCQDSLTLSLLKVFPQSSSWSGQNIFENIKDSDKMLSFRAHGPEVQAHNKRNLIQHNNSTQTDIFYTDRLEDRKEPGPPGGSSSFLHKPFPGGPLQVCPQACPSASERSLSSFRSDASGDRGFGLVDVRGRRPLLPFETEVGPCGVGEASLDKADSEGSNSGGTWPKAMLSSTAVPEKLSVYKKPKQRKSIFDPNTFKRPQTPPKIDYLLPGPGPAHSPQPSKRAGPLTPPKPPRRSDSIKFQHRLETSSESEATLVGSSPSTSPPSALPPDVDPGEPMHASPPRKARVRIASSYYPEGDGDSSHLPAKKSCDEDLTSQKVDELGQKRRRPKSAPSFRPKLAPVVIPAQFLEEQKCVPASGELSPELQEWAPYSPGHSSRHSNPPLYPSRPSVGTVPRSLTPSTTVSSILRNPIYTVRSHRVGPCSSPPAARDAGPQGLHPSVQHQGRLSLDLSHRTCSDYSEMRATHGSNSLPSSARLGSSSNLQFKAERIKIPSTPRYPRSVVGSERGSVSHSECSTPPQSPLNIDTLSSCSQSQTSASTLPRIAVNPASLGERRKDRPYVEEPRHVKVQKGSEPLGISIVSGEKGGIYVSKVTVGSIAHQAGLEYGDQLLEFNGINLRSATEQQARLIIGQQCDTITILAQYNPHVHQLSSHSRSSSHLDPAGTHSTLQGSGTTTPEHPSVIDPLMEQDEGPSTPPAKQSSSRIAGDANKKTLEPRVVFIKKSQLELGVHLCGGNLHGVFVAEVEDDSPAKGPDGLVPGDLILEYGSLDVRNKTVEEVYVEMLKPRDGVRLKVQYRPEEFTKAKGLPGDSFYIRALYDRLADVEQELSFKKDDILYVDDTLPQGTFGSWMAWQLDENAQKIQRGQIPSKYVMDQEFSRRLSMSEVKDDNSATKTLSAAARRSFFRRKHKHKRSGSKDGKDLLALDAFSSDSIPLFEDVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 109 - protein, 1161 aa, synthetic construct
MYGSARSVGKVEPSSQSPGRSPRLPRSPRLGHRRTNSTGGSSGSSVGGGSGKTLSMENIQSLNAAYATSGPMYLSDHENVGSETPKSTMTLGRSGGRLPYGVRMTAMGSSPNIASSGVASDTIAFGEHHLPPVSMASTVPHSLRQARDNTIMDLQTQLKEVLRENDLLRKDVEVKESKLSSSMNSIKTFWSPELKKERALRKDEASKITIWKEQYRVVQEENQHMQMTIQALQDELRIQRDLNQLFQQDSSSRTGEPCVAELTEENFQRLHAEHERQAKELFLLRKTLEEMELRIETQKQTLNARDESIKKLLEMLQSKGLSAKATEEDHERTRRLAEAEMHVHHLESLLEQKEKENSMLREEMHRRFENAPDSAKTKALQTVIEMKDSKISSMERGLRDLEEEIQMLKSNGALSTEEREEEMKQMEVYRSHSKFMKNKIGQVKQELSRKDTELLALQTKLETLTNQFSDSKQHIEVLKESLTAKEQRAAILQTEVDALRLRLEEKETMLNKKTKQIQDMAEEKGTQAGEIHDLKDMLDVKERKVNVLQKKIENLQEQLRDKEKQMSSLKERVKSLQADTTNTDTALTTLEEALAEKERTIERLKEQRDRDEREKQEEIDNYKKDLKDLKEKVSLLQGDLSEKEASLLDLKEHASSLASSGLKKDSRLKTLEIALEQKKEECLKMESQLKKAHEAALEARASPEMSDRIQHLEREITRYKDESSKAQAEVDRLLEILKEVENEKNDKDKKIAELERQVKDQNKKVANLKHKEQVEKKKSAQMLEEARRREDNLNDSSQQLQEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 110 - protein, 957 aa, synthetic construct
MYGSARSVGKVEPSSQSPGRSPRLPRSPRLGHRRTNSTGGSSGSSVGGGSGKTLSMENIQSLNAAYATSGPMYLSDHENVGSETPKSTMTLGRSGGRLPYGVRMTAMGSSPNIASSGVASDTIAFGEHHLPPVSMASTVPHSLRQARDNTIMDLQTQLKEVLRENDLLRKDVEVKESKLSSSMNSIKTFWSPELKKERALRKDEASKITIWKEQYRVVQEENQHMQMTIQALQDELRIQRDLNQLFQQDSSSRTGEPCVAELTEENFQRLHAEHERQAKELFLLRKTLEEMELRIETQKQTLNARDESIKKLLEMLQSKGLSAKATEEDHERTRRLAEAEMHVHHLESLLEQKEKENSMLREEMHRRFENAPDSAKTKALQTVIEMKDSKISSMERGLRDLEEEIQMLKSNGALSTEEREEEMKQMEVYRSHSKFMKNKIGQVKQELSRKDTELLALQTKLETLTNQFSDSKQHIEVLKESLTAKEQRAAILQTEVDALRLRLEEKETMLNKKTKQIQDMAEEKGTQAGEIHDLKDMLDVKERKVNVLQKKIENLQEQLRDKEKQMSSLKERVKSLQADTTNTDTALTTLEEALAEKEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 111 - protein, 1230 aa, synthetic construct
MYGSARSVGKVEPSSQSPGRSPRLPRSPRLGHRRTNSTGGSSGSSVGGGSGKTLSMENIQSLNAAYATSGPMYLSDHENVGSETPKSTMTLGRSGGRLPYGVRMTAMGSSPNIASSGVASDTIAFGEHHLPPVSMASTVPHSLRQARDNTIMDLQTQLKEVLRENDLLRKDVEVKESKLSSSMNSIKTFWSPELKKERALRKDEASKITIWKEQYRVVQEENQHMQMTIQALQDELRIQRDLNQLFQQDSSSRTGEPCVAELTEENFQRLHAEHERQAKELFLLRKTLEEMELRIETQKQTLNARDESIKKLLEMLQSKGLSAKATEEDHERTRRLAEAEMHVHHLESLLEQKEKENSMLREEMHRRFENAPDSAKTKALQTVIEMKDSKISSMERGLRDLEEEIQMLKSNGALSTEEREEEMKQMEVYRSHSKFMKNKIGQVKQELSRKDTELLALQTKLETLTNQFSDSKQHIEVLKESLTAKEQRAAILQTEVDALRLRLEEKETMLNKKTKQIQDMAEEKGTQAGEIHDLKDMLDVKERKVNVLQKKIENLQEQLRDKEKQMSSLKERVKSLQADTTNTDTALTTLEEALAEKERTIERLKEQRDRDEREKQEEIDNYKKDLKDLKEKVSLLQGDLSEKEASLLDLKEHASSLASSGLKKDSRLKTLEIALEQKKEECLKMESQLKKAHEAALEARASPEMSDRIQHLEREITRYKDESSKAQAEVDRLLEILKEVENEKNDKDKKIAELERQVKDQNKKVANLKHKEQVEKKKSAQMLEEARRREDNLNDSSQQLQIHCATSCAARSQPLSSSPSSSRCCCLPSASTATTSLPTSHPSPQLRPSGGPPRPSRSATPLPVPAGPRWTPWRTRSPWMPSRSWRIQSGNSLGRTWFLEKLEKANLEKWSRQRPSIKAEQGTPRWPRCKRTPPRVSCETCCQSSTSSRSTTHMSSNCMGPAARMARSSSSWSTPNTAPCGASSARAAKWGLATWAVEAAATPAPWTTRMSGPSPWATSSHLPGRSHRGCSIWPRSSFIGTWQPETSWLRGGRRFRISACPEMFMKRIPTRGARVGFQLNGWQLNPFLIISTPRKVMYGLLVSCCGRSPGETPILGFLLSGSSTFRPATGWRGQTTAARRCTACCNAGSRSRTKGRCLRTSAKTWRRWLRGETTWTLRRPLHLTPFMTTASQRRRHRWWTVIMPPSLEPSLPHGLKTNSMVEFPMHLLDS

SEQ ID NO: 112 - protein, 613 aa, synthetic construct
MAVQLVPDSALGLLMMTEGRRCQVHLLDDRKLELLVQPKLLAKELLDLVASHFNLKEKEYFGIAFTDETGHLNWLQLDRRVLEHDFPKKSGPVVLYFCVRFYIESISYLKDNATIELFFLNAKSCIYKELIDVDSEVVFELASYILQEAKGDFSSNEVVRSDLKKLPALPTQALKEHPSLAYCEDRVIEHYKKLNGQTRGQAIVNYMSIVESLPTYGVHYYAVKDKQGIPWWLGLSYKGIFQYDYHDKVKPRKEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 113 - protein, 900 aa, synthetic construct
MAAMAPGGSGSGGGVNPFLSDSDEDDDEVAATEERRAVLRLGAGSGLDPGSAGSLSPQDPVALGSSARPGLPGEASAAAVALGGTGETPARLSIDAIAAQLLRDQYLLTALELHTELLESGRELPRLRDYFSNPGNFERQSGTPPGMGAPGVPGAAGVGGAGGREPSTASGGGQLNRAGSISTLDSLDFARYSDDGNRETDEKVAVLEFELRKAKETIQALRANLTKAAEHEVPLQERKNYKSSPEIQEPIKPLEKRALNFLVNEFLLKNNYKLTSITFSDENDDQDFELWDDVGLNIPKPPDLLQLYRDFGNHQVTGKDLVDVASGVEEDELEALTPIISNLPPTLETPQPAENSMLVQKLEDKISLLNSEKWSLMEQIRRLKSEMDFLKNEHFAIPAVCDSVQPPLDQLPHKDSEDSGQHPDVNSSDKGKNTDIHLSISDEADSTIPKENSPNSFPRREREGMPPSSLSSKKTVHFDKPNRKLSPAFHQALLSFCRMSADSRLGYEVSRIADSEKSVMLMLGRCLPHIVPNVLLAKREEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 114 - protein, 926 aa, synthetic construct
MNTFQDQSGSSSNREPLLRCSDARRDLELAIGGVLRAEQQIKDNLREVKAQIHSCISRHLECLRSREVWLYEQVDLIYQLKEETLQQQAQQLYSLLGQFNCLTHQLECTQNKDLANQVSVCLERLGSLTLKPEDSTVLLFEADTITLRQTITTFGSLKTIQIPEHLMAHASSANIGPFLEKRGCISMPEQKSASGIVAVPFSEWLLGSKPASGYQAPYIPSTDPQDWLTQKQTLENSQTSSRACNFFNNVGGNLKGLENWLLKSEKSSYQKCNSHSTTSSFSIEMEKVGDQELPDQDEMDLSDWLVTPQESHKLRKPENGSRETSEKFKLLFQSYNVNDWLVKTDSCTNCQGNQPKGVEIENLGNLKCLNDHLEAKKPLSTPSMVTEDWLVQNHQDPCKVEEVCRANEPCTSFAECVCDENCEKEALYKWLLKKEGKDKNGMPVEPKPEPEKHKDSLNMWLCPRKEVIEQTKAPKAMTPSRIADSFQVIKNSPLSEWLIRPPYKEGSPKEVPGTEDRAGKQKFKSPMNTSWCSFNTADWVLPGKKMGNLSQLSSGEDKWLLRKKAQEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 115 - protein, 622 aa, synthetic construct
MKVTVCFGRTRVVVPCGDGHMKVFSLIQQAVTRYRKAIAKDPNYWIQVHRLEHGDGGILDLDDILCDVADDKDRMWPRRRAAPCPVQSARDGWSVRSVAAWAPQQAGVSGGKEMAKGSPGTSPPALPAPRPAPTATAMLWRPKTSTFALRTASGAALLGDTSLGSPGGLKLAMAPATASLRRRSASASPKTSRIHCATSCAARSQPLSSSPSSSRCCCLPSASTATTSLPTSHPSPQLRPSGGPPRPSRSATPLPVPAGPRWTPWRTRSPWMPSRSWRIQSGNSLGRTWFLEKLEKANLEKWSRQRPSIKAEQGTPRWPRCKRTPPRVSCETCCQSSTSSRSTTHMSSNCMGPAARMARSSSSWSTPNTAPCGASSARAAKWGLATWAVEAAATPAPWTTRMSGPSPWATSSHLPGRSHRGCSIWPRSSFIGTWQPETSWLRGGRRFRISACPEMFMKRIPTRGARVGFQLNGWQLNPFLIISTPRKVMYGLLVSCCGRSPGETPILGFLLSGSSTFRPATGWRGQTTAARRCTACCNAGSRSRTKGRCLRTSAKTWRRWLRGETTWTLRRPLHLTPFMTTASQRRRHRWWTVIMPPSLEPSLPHGLKTNSMVEFPMHLLDS

SEQ ID NO: 116 - protein, 554 aa, synthetic construct
MKVTVCFGRTRVVVPCGDGHMKVFSLIQQAVTRYRKAIAKDPNYWIQVHRLEHGDGGILDLDDILCDVADDKDRLVAVFDEQDPHHGGDGTSASSTGTQSPEIFGSELGTNNVSAFQPYQATSEIEVTPSVLRANMPLHVRRSSDPALIGLSTSVSDSNFSSEEPSRKNPTRWSTTAGFLKQNTAGSPKTCDRKEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 117 - protein, 596 aa, synthetic construct
MESGSTAASEEARSLRECELYVQKHNIQALLKDSIVQLCTARPERPMAFLREYFERLEKEEAKQIQNLQKAGTRTDSREDEISPPPPNPVVKGRRRRGAISAEVYTEEDAASYVRKVIPKDYKTMAALAKAIEKNVLFSHLDDNERSDIFDAMFSVSFIAGETVIQQGDEGDNFYVIDQGETDVYVNNEWATSVGEGGSFGELALIYGTPRAATVKAKTNVKLWGIDRDSYRRILMEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 118 - protein, 1148 aa, synthetic construct
MGTLRDLQYALQEKIEELRQRDALIDELELELDQKDELIQKLQNELDKYRSVIRPATQQAQKQSASTLQGEPRTKRQAISAEPTAFDIQDLSHVTLPFYPKSPQSKDLIKEAILDNDFMKNLELSQIQEIVDCMYPVEYGKDSCIIKEGDVGSLVYVMEDGKVEVTKEGVKLCTMGPGKVFGELAILYNCTRTATVKTLVNVKLWAIDRQCFQTIMMRTGLIKHTEYMEFLKSVPTFQSLPEEILSKLADVLEETHYENGEYIIRQGARGDTFFIISKGTVNVTREDSPSEDPVFLRTLGKGDWFGEKALQGGSALPLRPGQPGGEHALGPGPRAAGEVRAGGRVHRARRRARGGGDGALPGDRVRRGRLGAHLPRGRRHRQRRGGVQAEGGHRGGHAACLRCRRGTCIRGAGEAVHKHAAPRGHLGPADLPGGTLAQRDLGPGQRQLRAGDRTLAGSQPEPLHLGEPHHAAGGAGQLRLPGPRSGRPLAPLQRVGAAGQPAPAQYLLPLREQEGSPICPDRESLCGKLPGIQWHQRPVQAAFLWCQLQHARGGHLSRGHLGDPVCEHQGPAAAQVCRTSLHGGGHRPADLAGPGPAACNSGGVICGRGGGLPPVLCSQQETAGVGVWRPGLPNRQVVEARRWQRDHQELLHLLSQHQDLPRRPLRCCGDPRHQHLPSGLPPGQHCWGTRAWGAPGDSWLWHLQLLPGGEVLLRARRHPGSTVRRAVPHGDRSRCPLLLHRLGAAVCLLHPLLPQVCPQATHLLSDDLPEARPGLPGQLLLFRCPPALAGLHGEPGLRGCLQDPGGSKVGIPSEELGSWKNSRRRRIWKSGQGNGLPSERQSRVHHGGREDAERERLPEAARPAVRVQRPEAGQPPTCHQIVWGLQPGWPAPPHRGVRQIRLPAGLPPREPQSGAWLPGQWRQPQLQLPGPPGAGPHHGRPHLICLADLTGDAVSGRDEARSSGLGSQKHPGSGAEDEDFGFRLVPRCLRGFLREEEPGSDSSMDGNIPFSYLHHAKCMVFWCPAVGDRDPRGKPLSWDSSAALQPSEDRPPDGEARQLQRGDVPPDAAMLEAGAGQKAGVCGHQQRPGEDDGEERLLGPCGVHSILPDLRRPLRGGDTAGGLCPPPSSPPFHMDKQTLWNFPCIYIL

SEQ ID NO: 119 - protein, 339 aa, synthetic construct
MGTLRDLQYALQEKIEELRQRDALIDELELELDQKDELIQKLQNELDKYRSVIRPATQQAQKQSASTLQGEPRTKRQAISAEPTAFDIQDLSHVTLPFYPKSPQSKDLIKEAILDNDFMKNLELSQIQEIVDCMYPVEYGKDSCIIKEGDVGSLVYVMEDGKVEVTKEGVKLCTMGPGKVFGELAILYNCTRTATVKTLVNVKLWAIDRQCFQTIMMRTGLIKHTEYMEFLKSVPTFQSLPEEILSKLADVLEEVPPDAAMLEAGAGQKAGVCGHQQRPGEDDGEERLLGPCGVHSILPDLRRPLRGGDTAGGLCPPPSSPPFHMDKQTLWNFPCIYIL

SEQ ID NO: 120 - protein, 669 aa, synthetic construct
MTFQVWGWRREDASQVLAWVPDAEGGRRGMLTRRSLATKDPTAVERANLLNMAKLSIKGLIESALSFGRTLDSDYPPLQQFFVVMEHCLKHGLKVRKSFLSYNKTIWGPLELVEKLYPEAEEIGASVRDLPGLKTPLGRARAWLRLALMQKKMADYLRCLIIQRDLLSEFYEYHALMMEEEGAVIVGLLVGLNVIDANLCVKGEDLDSQVGVIDFSMYLKNEEDIGNKERNVQIAAILDQKNYVEELNRQLNSTVSSLHSRVDSLEKSNTKLIEELAIAKNNIIKLQEENHQLRSENKLILMKTQQHLEEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 121 - protein, 491 aa, synthetic construct
MTQFLPPNLLALFAPRDPIPYLPPLEKLPHEKHHNQPYCGIAPYIREFEDPRDAPPPTRAETREERMERKRREKIERRQQEVETELKMWDPHNDPNAQGDAFKTLFVARVNYDTTESKLRREFEVYGPIKREDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 122 - protein, 697 aa, synthetic construct
MASLTVKAYLLGKEDAAREIRRFSFCCSPEPEAEAEAAAGPGPCERLLSRVAALFPALRPGGFQAHYRDEDGDLVAFSSDEELTMAMSYVKDDIFRIYIKEKKECRRDHRPPCAQEAPRNMVHPNVICDGCNGPVVGTRYKCSVCPDYDLCSVCEGKGLHRGHTKLAFPSPFGHLSEGFSHSRWLRKVKHGHFGWPGWEMGPPGNWSPRPPRAGEARPGPTAESASGPSEDPSVNFLKNVGESVAAALSPLDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 123 - protein, 825 aa, synthetic construct
MEGRGPYRIYDPGGSVPSGEASAAFERLVKENSRLKEKMQGIKMLGELLEESQMEATRLRQKAEELVKDNELLPPPSPSLGSFDPLAELTGKDSNVTASPTAPACPSDKPAPVQKPPSSGTSSEFEVVTPEEQNSPESSSHANAMALGPLPREDGNLMLHLQRLETTLSVCAEEPDHGQLFTHLGRMALEFNRLASKVHKNEQRTSILQTLCEQLRKENEALKAKLDKGLEQRDQAAERLREENLELKKLLMSNGNKEGASGRPGSPKMEGTGKKAVAGQQQASVTAGKVPEVVALGAAEKKVKMLEQQRSELLEVNKQWDQHFRSMKQQYEQKITELRQKLADLQKQVTDLEAEREQKQRDFDRKLLLAKSKIEMEETDKEQLTAEAKELRQKVKYLQDQLSPLTRQREYQEKEIQRLNKALEEALSIQTPPSSPPTAFGSPEGAGALLRKQELVTQNELLKQQEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 124 - protein, 953 aa, synthetic construct
MEGRGPYRIYDPGGSVPSGEASAAFERLVKENSRLKEKMQGIKMLGELLEESQMEATRLRQKAEELVKDNELLPPPSPSLGSFDPLAELTGKDSNVTASPTAPACPSDKPAPVQKPPSSGTSSEFEVVTPEEQNSPESSSHANAMALGPLPREDGNLMLHLQRLETTLSVCAEEPDHGQLFTHLGRMALEFNRLASKVHKNEQRTSILQTLCEQLRKENEALKAKLDKGLEQRDQAAERLREENLELKKLLMSNGNKEGASGRPGSPKMEGTGKKAVAGQQQASVTAGKVPEVVALGAAEKKVKMLEQQRSELLEVNKQWDQHFRSMKQQYEQKITELRQKLADLQKQVTDLEAEREQKQRDFDRKLLLAKSKIEMEETDKEQLTAEAKELRQKVKYLQDQLSPLTRQREYQEKEIQRLNKALEEALSIQTPPSSPPTAFGSPEGAGALLRKQELVTQNELLKQQVKIFEEDFQRERSDRERMNEEKEELKKQVEKLQAQVTLSNAQLKAFKDEEKAREALRQQKRKAKASGERYHVEPHPEHLCGAYPYAYPPMPAMVPHHGFEDWSQIRYPPPPMAMEHPPPLPNSRLFHLEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 125 - protein, 781 aa, synthetic construct
MEGRGPYRIYDPGGSVPSGEASAAFERLVKENSRLKEKMQGIKMLGELLEESQMEATRLRQKAEELVKDNELLPPPSPSLGSFDPLAELTGKDSNVTASPTAPACPSDKPAPVQKPPSSGTSSEFEVVTPEEQNSPESSSHANAMALGPLPREDGNLMLHLQRLETTLSVCAEEPDHGQLFTHLGRMALEFNRLASKVHKNEQRTSILQTLCEQLRKENEALKAKLDKGLEQRDQAAERLREENLELKKLLMSNGNKEGASGRPGSPKMEGTGKKAVAGQQQASVTAGKVPEVVALGAAEKKVKMLEQQRSELLEVNKQWDQHFRSMKQQYEQKITELRQKLADLQKQVTDLEAEREQKQRDFDRKLLLAKSKIEMEETDKEQLTAEAKELRQKVKYLQDQLSPLTRQREYQEKEIQRLNKEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 126 - protein, 609 aa, synthetic construct
MASGSVAECLQQETTCPVCLQYFAEPMMLDCGHNICCACLARCWGTAETNVSCPQCRETFPQRHMRPNRHLANVTQLVKQLRTERPSGPGGEMGVCEKHREPLKLYCEEDQMPICVVCDRSREHRGHSVLPLEEAVEGFKEQIQNQLDHLKRVKDLKKRRRAQGEQARAELLSLTQMEREKIVWEFEQLYHSLKEHEYRLLARLEELDLAIYNSINGAITQFSCNISHLSSLIAQLEEKQQQPTRELLQEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 127 - protein, 980 aa, synthetic construct
MAENKGGGEAESGGGGSGSAPVTAGAAGPAAQEAEPPLTAVLVEEEEEEGGRAGAEGGAAGPDDGGVAAASSGSAQAASSPAASVGTGVAGGAVSTPAPAPASAPAPGPSAGPPPGPPASLLDTCAVCQQSLQSRREAEPKLLPCLHSFCLRCLPEPERQLSVPIPGGSNGDIQQVGVIRCPVCRQECRQIDLVDNYFVKDTSEAPSSSDEKSEQVCTSCEDNASAVGFCVECGEWLCKTCIEAHQRVKFTKDHLIRKKEDVSESVGASGQRPVFCPVHKQEQLKLFCETCDRLTCRDCQLLEHKEHRYQFLEEAFQNQKGAIENLLAKLLEKKNYVHFAATQVQNRIKEVNETNKRVEQEIKVAIFTLINEINKKGKSLLQQLENVTKERQMKLLQQQNDITGLSRQVKHVMNFTNWAIASGSSTALLYSKRLITFQLRHILKARCDPVPAANGAIRFHCDPTFWAKNVVNLGNLVIESKPAPGYTPNVVVGQVPPGTNHISKTPGQINLAQLRLQHMQQQVYAQKHQQLQQMRMQQPPAPVPTTTTTTQQHPRQAAPQMLQQQPPRLISVQTMQRGNMNCGAFQAHQMRLAQNAARIPGIPRHSGPQYSMMQPHLQRQEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 128 - protein, 623 aa, synthetic construct
MADSASESDTDGAGGNSSSSAAMQSSCSSTSGGGGGGGGGGGGGKSGGIVISPFRLEELTNRLASLQQENKVLKIELETYKLKCKALQEENRDLRKASVTIQARAEQEEEFISNTLFKKIQALQKEKETLAVNYEKEEEFLTNELSRKLMQLQHEKAELEQHLEQEQEFQVNKLMKKIKKLENDTISKQLTLEQIHCATSCAARSQPLSSSPSSSRCCCLPSASTATTSLPTSHPSPQLRPSGGPPRPSRSATPLPVPAGPRWTPWRTRSPWMPSRSWRIQSGNSLGRTWFLEKLEKANLEKWSRQRPSIKAEQGTPRWPRCKRTPPRVSCETCCQSSTSSRSTTHMSSNCMGPAARMARSSSSWSTPNTAPCGASSARAAKWGLATWAVEAAATPAPWTTRMSGPSPWATSSHLPGRSHRGCSIWPRSSFIGTWQPETSWLRGGRRFRISACPEMFMKRIPTRGARVGFQLNGWQLNPFLIISTPRKVMYGLLVSCCGRSPGETPILGFLLSGSSTFRPATGWRGQTTAARRCTACCNAGSRSRTKGRCLRTSAKTWRRWLRGETTWTLRRPLHLTPFMTTASQRRRHRWWTVIMPPSLEPSLPHGLKTNSMVEFPMHLLDS

SEQ ID NO: 129 - protein, 1257 aa, synthetic construct
MEVAVEKAVAAAAAASAAASGGPSAAPSGENEAESRQGPDSERGGEAARLNLLDTCAVCHQNIQSRAPKLLPCLHSFCQRCLPAPQRYLMLPAPMLGSAETPPPVPAPGSPVSGSSPFATQVGVIRCPVCSQECAERHIIDNFFVKDTTEVPSSTVEKSNQVCTSCEDNAEANGFCVECVEWLCKTCIRAHQRVKFTKDHTVRQKEEVSPEAVGVTSQRPVFCPFHKKEQLKLYCETCDKLTCRDCQLLEHKEHRYQFIEEAFQNQKVIIDTLITKLMEKTKYIKFTGNQIQNRIIEVNQNQKQVEQDIKVAIFTLMVEINKKGKALLHQLESLAKDHRMKLMQQQQEVAGLSKQLEHVMHFSKWAVSSGSSTALLYSKRLITYRLRHLLRARCDASPVTNNTIQFHCDPSFWAQNIINLGSLVIEDKESQPQMPKQNPVVEQNSQPPSGLSSNQLSKFPTQISLAQLRLQHMQQQQPPPRLINFQNHSPKPNGPVLPPHPQQLRYPPNQNIPRQAIKPNPLQMAFLAQQAIKQWQISSGQGTPSTTNSTSSTPSSPTITSAAGYDGKAFGSPMIDLSSPVGGSYNLPSLPDIDCSSTIMLDNIVRKDTNIDHGQPRPPSNRTVQSPNSSVPSPGLAGPVTMTSVHPPIRSPSASSVGSRGSSGSSSKPAGADSTHKVPVVMLEPIRIKQENSGPPENYDFPVVIVKQESDEESRPQNANYPRSILTSLLLNSSQSSTSEETVLRSDAPDSTGDQPGLHQDNSSNGKSEWLDPSQKSPLHVGETRKEDDPNEDWCAVCQNGGELLCCEKCPKVFHLSCHVPTLTNFPSGEWICTFCRDLSKPEVEYDCDAPSHNSEKKKTEGLVKLTPIDKRKCERLLLFLYCHEMSLAFQDPVPLTEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 130 - protein, 533 aa, synthetic construct
MAATAAAVVAEEDTELRDLLVQTLENSGVLNRIKAELRAAVFLALEEQEKVENKTPLVNESLKKFLNTKDGRLVASLVAEFLQFFNLDFTLAVFQPETSTLQGLEGRENLARDLGIIEAEGTVGGPLLLEVIRRCQQKEKGPTTGEGALDLSDVHSPPKSPEGKTSAQTTPSKEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 131 - protein, 1080 aa, synthetic construct
MCTALSPKVRSGPGLSDMHQYSQWLASRHEANLLPMKEDLALWLTNLLGKEITAETFMEKLDNGALLCQLAETMQEKFKESMDANKPTKNLPLKKIPCKTSAPSGSFFARDNTANFLSWCRDLGVDETCLFESEGLVLHKQPREVCLCLLELGRIAARYGVEPPGLIKLEKEIEQEETLSAPSPSPSPSSKSSGKKSTGNLLDDAVKRISEDPPCKCPNKFCVERLSQGRYRVGEKILFIRVRVCPSAAPRTAWRARAGPWTASSGRSTSWWPCAPCTPARARRWWCPSRPCTTRTTRRPPSPRASTPPAPWWSSSGRRTPWWPRCVSSMQTWYLHQGSWGGTQARCSPGTPGPSRPSGWNTGPTRPRSRPTAASCGRPYMTIGWFSTGTSPSRRTAPCSWRCWSMTQTSRAQERASSCSTSTCRCCRSACTCPVPTPSPAGGLADLPRSGKSVWKTARHSVASTSSTSCIPLVPTAARGWSPQPRTPRGSCLMTPRPCGGPSVPNFTTWWWPPTSRPLGRPRPSCLQWRGHMWPRRRAAPCPVQSARDGWSVRSVAAWAPQQAGVSGGKEMAKGSPGTSPPALPAPRPAPTATAMLWRPKTSTFALRTASGAALLGDTSLGSPGGLKLAMAPATASLRRRSASASPKTSRIHCATSCAARSQPLSSSPSSSRCCCLPSASTATTSLPTSHPSPQLRPSGGPPRPSRSATPLPVPAGPRWTPWRTRSPWMPSRSWRIQSGNSLGRTWFLEKLEKANLEKWSRQRPSIKAEQGTPRWPRCKRTPPRVSCETCCQSSTSSRSTTHMSSNCMGPAARMARSSSSWSTPNTAPCGASSARAAKWGLATWAVEAAATPAPWTTRMSGPSPWATSSHLPGRSHRGCSIWPRSSFIGTWQPETSWLRGGRRFRISACPEMFMKRIPTRGARVGFQLNGWQLNPFLIISTPRKVMYGLLVSCCGRSPGETPILGFLLSGSSTFRPATGWRGQTTAARRCTACCNAGSRSRTKGRCLRTSAKTWRRWLRGETTWTLRRPLHLTPFMTTASQRRRHRWWTVIMPPSLEPSLPHGLKTNSMVEFPMHLLDS

SEQ ID NO: 132 - protein, 1009 aa, synthetic construct
MEETQPPPQPKLPLCDSLMIWLQTFNTASPCQDVKQLTSGVAMAQVLHQIDAAWFNESWLSRIKEDVGDNWRIKASNVKKVLQGIMSYYHEFLGQQISEALIPDLNQITECSDPVELGRLLQLILGCAINCEKKQEHIQNIMTLEESVQHVVMTAIQELMSKEILSSPPNDAVGELEQQLKRALEELQEALAEKEELRQRCEELDMQVTTLQDEKNSLVSENEMMNEKLDQLDGSFDDPNTVVAKKYFHAQLQLEQLQEENFRLEAAKDDYRVHCEELEKQLIEFQHRNDELTSLAEETRALKDEIDVLRATSDKANKLESTVEIYRQKLQDLNDLRKQVKTLQETNMMYMHNTVSLEEELKKANAARTQLETYKRQVQDLHVKLSSESKRADTLAFEMKRLEEKHEALLKEKERLIEQRDTLKETNEELRCSQVQQDHLNQTDASATKSYENLAAEIMPVEYREVFIRLQHENKMLRLQQEGSENERIEELQEQLEQKHRKMNELETEQRLSKERIRELQQQIEDLQKSLQEQGSKSEGESSSKLKQKLEAHMEKLTEVHEELQKKQELIEDLQPDINQNVQKINELEAALQKKDEDMKAMEERYKMYLEKARNVIKTLDPKLNPASAEIMLLRKQLAEKERRIEILEEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 133 - protein, 3219 aa, Homo sapiens
ATGGCGAAGGCGACGTCCGGTGCCGCGGGGCTGCGTCTGCTGTTGCTGCTGCTGCTGCCGCTGCTAGGCAAAGTGGCATTGGGCCTCTACTTCTCGAGGGATGCTTACTGGGAGAAGCTGTATGTGGACCAGGCAGCCGGCACGCCCTTGCTGTACGTCCATGCCCTGCGGGACGCCCCTGAGGAGGTGCCCAGCTTCCGCCTGGGCCAGCATCTCTACGGCACGTACCGCACACGGCTGCATGAGAACAACTGGATCTGCATCCAGGAGGACACCGGCCTCCTCTACCTTAACCGGAGCCTGGACCATAGCTCCTGGGAGAAGCTCAGTGTCCGCAACCGCGGCTTTCCCCTGCTCACCGTCTACCTCAAGGTCTTCCTGTCACCCACATCCCTTCGTGAGGGCGAGTGCCAGTGGCCAGGCTGTGCCCGCGTATACTTCTCCTTCTTCAACACCTCCTTTCCAGCCTGCAGCTCCCTCAAGCCCCGGGAGCTCTGCTTCCCAGAGACAAGGCCCTCCTTCCGCATTCGGGAGAACCGACCCCCAGGCACCTTCCACCAGTTCCGCCTGCTGCCTGTGCAGTTCTTGTGCCCCAACATCAGCGTGGCCTACAGGCTCCTGGAGGGTGAGGGTCTGCCCTTCCGCTGCGCCCCGGACAGCCTGGAGGTGAGCACGCGCTGGGCCCTGGACCGCGAGCAGCGGGAGAAGTACGAGCTGGTGGCCGTGTGCACCGTGCACGCCGGCGCGCGCGAGGAGGTGGTGATGGTGCCCTTCCCGGTGACCGTGTACGACGAGGACGACTCGGCGCCCACCTTCCCCGCGGGCGTCGACACCGCCAGCGCCGTGGTGGAGTTCAAGCGGAAGGAGGACACCGTGGTGGCCACGCTGCGTGTCTTCGATGCAGACGTGGTACCTGCATCAGGGGAGCTGGTGAGGCGGTACACAAGCACGCTGCTCCCCGGGGACACCTGGGCCCAGCAGACCTTCCGGGTGGAACACTGGCCCAACGAGACCTCGGTCCAGGCCAACGGCAGCTTCGTGCGGGCGACCGTACATGACTATAGGCTGGTTCTCAACCGGAACCTCTCCATCTCGGAGAACCGCACCATGCAGCTGGCGGTGCTGGTCAATGACTCAGACTTCCAGGGCCCAGGAGCGGGCGTCCTCTTGCTCCACTTCAACGTGTCGGTGCTGCCGGTCAGCCTGCACCTGCCCAGTACCTACTCCCTCTCCGTGAGCAGGAGGGCTCGCCGATTTGCCCAGATCGGGAAAGTCTGTGTGGAAAACTGCCAGGCATTCAGTGGCATCAACGTCCAGTACAAGCTGCATTCCTCTGGTGCCAACTGCAGCACGCTAGGGGTGGTCACCTCAGCCGAGGACACCTCGGGGATCCTGTTTGTGAATGACACCAAGGCCCTGCGGCGGCCCAAGTGTGCCGAACTTCACTACATGGTGGTGGCCACCGACCAGCAGACCTCTAGGCAGGCCCAGGCCCAGCTGCTTGTAACAGTGGAGGGGTCATATGTGGCCGAGGAGGCGGGCTGCCCCCTGTCCTGTGCAGTCAGCAAGAGACGGCTGGAGTGTGAGGAGTGTGGCGGCCTGGGCTCCCCAACAGGCAGGTGTGAGTGGAGGCAAGGAGATGGCAAAGGGATCACCAGGAACTTCTCCACCTGCTCTCCCAGCACCAAGACCTGCCCCGACGGCCACTGCGATGTTGTGGAGACCCAAGACATCAACATTTGCCCTCAGGACTGCCTCCGGGGCAGCATTGTTGGGGGACACGAGCCTGGGGAGCCCCGGGGGATTAAAGCTGGCTATGGCACCTGCAACTGCTTCCCTGAGGAGGAGAAGTGCTTCTGCGAGCCCGAAGACATCCAGGATCCACTGTGCGACGAGCTGTGCCGCACGGTGATCGCAGCCGCTGTCCTCTTCTCCTTCATCGTCTCGGTGCTGCTGTCTGCCTTCTGCATCCACTGCTACCACAAGTTTGCCCACAAGCCACCCATCTCCTCAGCTGAGATGACCTTCCGGAGGCCCGCCCAGGCCTTCCCGGTCAGCTACTCCTCTTCCGGTGCCCGCCGGCCCTCGCTGGACTCCATGGAGAACCAGGTCTCCGTGGATGCCTTCAAGATCCTGGAGGATCCAAAGTGGGAATTCCCTCGGAAGAACTTGGTTCTTGGAAAAACTCTAGGAGAAGGCGAATTTGGAAAAGTGGTCAAGGCAACGGCCTTCCATCTGAAAGGCAGAGCAGGGTACACCACGGTGGCCGTGAAGATGCTGAAAGAGAACGCCTCCCCGAGTGAGCTGCGAGACCTGCTGTCAGAGTTCAACGTCCTGAAGCAGGTCAACCACCCACATGTCATCAAATTGTATGGGGCCTGCAGCCAGGATGGCCCGCTCCTCCTCATCGTGGAGTACGCCAAATACGGCTCCCTGCGGGGCTTCCTCCGCGAGAGCCGCAAAGTGGGGCCTGGCTACCTGGGCAGTGGAGGCAGCCGCAACTCCAGCTCCCTGGACCACCCGGATGAGCGGGCCCTCACCATGGGCGACCTCATCTCATTTGCCTGGCAGATCTCACAGGGGATGCAGTATCTGGCCGAGATGAAGCTCGTTCATCGGGACTTGGCAGCCAGAAACATCCTGGTAGCTGAGGGGCGGAAGATGAAGATTTCGGATTTCGGCTTGTCCCGAGATGTTTATGAAGAGGATTCCTACGTGAAGAGGAGCCAGGGTCGGATTCCAGTTAAATGGATGGCAATTGAATCCCTTTTTGATCATATCTACACCACGCAAAGTGATGTATGGTCTTTTGGTGTCCTGCTGTGGGAGATCGTGACCCTAGGGGGAAACCCCTATCCTGGGATTCCTCCTGAGCGGCTCTTCAACCTTCTGAAGACCGGCCACCGGATGGAGAGGCCAGACAACTGCAGCGAGGAGATGTACCGCCTGATGCTGCAATGCTGGAAGCAGGAGCCGGACAAAAGGCCGGTGTTTGCGGACATCAGCAAAGACCTGGAGAAGATGATGGTTAAGAGGAGAGACTACTTGGACCTTGCGGCGTCCACTCCATCTGACTCCCTGATTTATGACGACGGCCTCTCAGAGGAGGAGACACCGCTGGTGGACTGTAATAATGCCCCCCTCCCTCGAGCCCTCCCTTCCACATGGATTGAAAACAAACTCTATGGTAGAATTTCCCATGCATTTACTAGATTCTAG

SEQ ID NO: 134 - protein, 3345 aa, Homo sapiens
ATGGCGAAGGCGACGTCCGGTGCCGCGGGGCTGCGTCTGCTGTTGCTGCTGCTGCTGCCGCTGCTAGGCAAAGTGGCATTGGGCCTCTACTTCTCGAGGGATGCTTACTGGGAGAAGCTGTATGTGGACCAGGCAGCCGGCACGCCCTTGCTGTACGTCCATGCCCTGCGGGACGCCCCTGAGGAGGTGCCCAGCTTCCGCCTGGGCCAGCATCTCTACGGCACGTACCGCACACGGCTGCATGAGAACAACTGGATCTGCATCCAGGAGGACACCGGCCTCCTCTACCTTAACCGGAGCCTGGACCATAGCTCCTGGGAGAAGCTCAGTGTCCGCAACCGCGGCTTTCCCCTGCTCACCGTCTACCTCAAGGTCTTCCTGTCACCCACATCCCTTCGTGAGGGCGAGTGCCAGTGGCCAGGCTGTGCCCGCGTATACTTCTCCTTCTTCAACACCTCCTTTCCAGCCTGCAGCTCCCTCAAGCCCCGGGAGCTCTGCTTCCCAGAGACAAGGCCCTCCTTCCGCATTCGGGAGAACCGACCCCCAGGCACCTTCCACCAGTTCCGCCTGCTGCCTGTGCAGTTCTTGTGCCCCAACATCAGCGTGGCCTACAGGCTCCTGGAGGGTGAGGGTCTGCCCTTCCGCTGCGCCCCGGACAGCCTGGAGGTGAGCACGCGCTGGGCCCTGGACCGCGAGCAGCGGGAGAAGTACGAGCTGGTGGCCGTGTGCACCGTGCACGCCGGCGCGCGCGAGGAGGTGGTGATGGTGCCCTTCCCGGTGACCGTGTACGACGAGGACGACTCGGCGCCCACCTTCCCCGCGGGCGTCGACACCGCCAGCGCCGTGGTGGAGTTCAAGCGGAAGGAGGACACCGTGGTGGCCACGCTGCGTGTCTTCGATGCAGACGTGGTACCTGCATCAGGGGAGCTGGTGAGGCGGTACACAAGCACGCTGCTCCCCGGGGACACCTGGGCCCAGCAGACCTTCCGGGTGGAACACTGGCCCAACGAGACCTCGGTCCAGGCCAACGGCAGCTTCGTGCGGGCGACCGTACATGACTATAGGCTGGTTCTCAACCGGAACCTCTCCATCTCGGAGAACCGCACCATGCAGCTGGCGGTGCTGGTCAATGACTCAGACTTCCAGGGCCCAGGAGCGGGCGTCCTCTTGCTCCACTTCAACGTGTCGGTGCTGCCGGTCAGCCTGCACCTGCCCAGTACCTACTCCCTCTCCGTGAGCAGGAGGGCTCGCCGATTTGCCCAGATCGGGAAAGTCTGTGTGGAAAACTGCCAGGCATTCAGTGGCATCAACGTCCAGTACAAGCTGCATTCCTCTGGTGCCAACTGCAGCACGCTAGGGGTGGTCACCTCAGCCGAGGACACCTCGGGGATCCTGTTTGTGAATGACACCAAGGCCCTGCGGCGGCCCAAGTGTGCCGAACTTCACTACATGGTGGTGGCCACCGACCAGCAGACCTCTAGGCAGGCCCAGGCCCAGCTGCTTGTAACAGTGGAGGGGTCATATGTGGCCGAGGAGGCGGGCTGCCCCCTGTCCTGTGCAGTCAGCAAGAGACGGCTGGAGTGTGAGGAGTGTGGCGGCCTGGGCTCCCCAACAGGCAGGTGTGAGTGGAGGCAAGGAGATGGCAAAGGGATCACCAGGAACTTCTCCACCTGCTCTCCCAGCACCAAGACCTGCCCCGACGGCCACTGCGATGTTGTGGAGACCCAAGACATCAACATTTGCCCTCAGGACTGCCTCCGGGGCAGCATTGTTGGGGGACACGAGCCTGGGGAGCCCCGGGGGATTAAAGCTGGCTATGGCACCTGCAACTGCTTCCCTGAGGAGGAGAAGTGCTTCTGCGAGCCCGAAGACATCCAGGATCCACTGTGCGACGAGCTGTGCCGCACGGTGATCGCAGCCGCTGTCCTCTTCTCCTTCATCGTCTCGGTGCTGCTGTCTGCCTTCTGCATCCACTGCTACCACAAGTTTGCCCACAAGCCACCCATCTCCTCAGCTGAGATGACCTTCCGGAGGCCCGCCCAGGCCTTCCCGGTCAGCTACTCCTCTTCCGGTGCCCGCCGGCCCTCGCTGGACTCCATGGAGAACCAGGTCTCCGTGGATGCCTTCAAGATCCTGGAGGATCCAAAGTGGGAATTCCCTCGGAAGAACTTGGTTCTTGGAAAAACTCTAGGAGAAGGCGAATTTGGAAAAGTGGTCAAGGCAACGGCCTTCCATCTGAAAGGCAGAGCAGGGTACACCACGGTGGCCGTGAAGATGCTGAAAGAGAACGCCTCCCCGAGTGAGCTGCGAGACCTGCTGTCAGAGTTCAACGTCCTGAAGCAGGTCAACCACCCACATGTCATCAAATTGTATGGGGCCTGCAGCCAGGATGGCCCGCTCCTCCTCATCGTGGAGTACGCCAAATACGGCTCCCTGCGGGGCTTCCTCCGCGAGAGCCGCAAAGTGGGGCCTGGCTACCTGGGCAGTGGAGGCAGCCGCAACTCCAGCTCCCTGGACCACCCGGATGAGCGGGCCCTCACCATGGGCGACCTCATCTCATTTGCCTGGCAGATCTCACAGGGGATGCAGTATCTGGCCGAGATGAAGCTCGTTCATCGGGACTTGGCAGCCAGAAACATCCTGGTAGCTGAGGGGCGGAAGATGAAGATTTCGGATTTCGGCTTGTCCCGAGATGTTTATGAAGAGGATTCCTACGTGAAGAGGAGCCAGGGTCGGATTCCAGTTAAATGGATGGCAATTGAATCCCTTTTTGATCATATCTACACCACGCAAAGTGATGTATGGTCTTTTGGTGTCCTGCTGTGGGAGATCGTGACCCTAGGGGGAAACCCCTATCCTGGGATTCCTCCTGAGCGGCTCTTCAACCTTCTGAAGACCGGCCACCGGATGGAGAGGCCAGACAACTGCAGCGAGGAGATGTACCGCCTGATGCTGCAATGCTGGAAGCAGGAGCCGGACAAAAGGCCGGTGTTTGCGGACATCAGCAAAGACCTGGAGAAGATGATGGTTAAGAGGAGAGACTACTTGGACCTTGCGGCGTCCACTCCATCTGACTCCCTGATTTATGACGACGGCCTCTCAGAGGAGGAGACACCGCTGGTGGACTGTAATAATGCCCCCCTCCCTCGAGCCCTCCCTTCCACATGGATTGAAAACAAACTCTATGGCATGTCAGACCCGAACTGGCCTGGAGAGAGTCCTGTACCACTCACGAGAGCTGATGGCACTAACACTGGGTTTCCAAGATATCCAAATGATAGTGTATATGCTAACTGGATGCTTTCACCCTCAGCGGCAAAATTAATGGACACGTTTGATAGTTAA

SEQ ID NO: 135 - protein, 1072 aa, Homo sapiens
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 136 - protein, 1114 aa, Homo sapiens
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

SEQ ID NO: 137 - protein, 531 aa, synthetic construct
METPSQRRATRSGAQASSTPLSPTRITRLQEKEDLQELNDRLAVYIDRVRSLETENAGLRLRITESEEVVSREVSGIKAAYEAELGDARKTLDSVAKERARLQLELSKVREEFKELKARNTKKEGDLIAAQARLKDLEALLNSKEAALSTALSEKRTLEGELHDLRGQVAKEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 138 - protein, 531 aa, synthetic construct; identical to SEQ ID NO: 137

SEQ ID NO: 139 - protein, 728 aa, synthetic construct
MGKKLDLSKLTDEEAQHVLEVVQRDFDLRRKEEERLEALKGKIKKESSKRELLSDTAHLNETHCARCLQPYQLLVNSKRQCLECGLFTCKSCGRVHPEEQGWICDPCHLARVVKIGSLEWYYEHVKARFKRFGSAKVIRSLHGRLQGGAGPELISEERSGDSDQTDEDGEPGSEAQAQAQPFGSKKKRLLSVHDFDFEGDSDDSTQPQGHSLHLSSVPEARDSPQSLTDESCSEKAAPHKAEGLEEADTGASGCHSHPEEQPTSISPSRHGALAELCPPGGSHRMALGTAAALGSNVIRNEQLPLQYLADVDTSDEESIRAHVMASHHSKRRGRASSESQIFELNKHISAVECLLTYLENTVVPPLAKEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 140 - protein, 2004 aa, synthetic construct
MAQQAADKYLYVDKNFINNPLAQADWAAKKLVWVPSDKSGFEPASLKEEVGEEAIVELVENGKKVKVNKDDIQKMNPPKFSKVEDMAELTCLNEASVLHNLKERYYSGLIYTYSGLFCVVINPYKNLPIYSEEIVEMYKGKKRHEMPPHIYAITDTAYRSMMQDREDQSILCTGESGAGKTENTKKVIQYLAYVASSHKSKKDQGELERQLLQANPILEAFGNAKTVKNDNSSRFGKFIRINFDVNGYIVGANIETYLLEKSRAIRQAKEERTFHIFYYLLSGAGEHLKTDLLLEPYNKYRFLSNGHVTIPGQQDKDMFQETMEAMRIMGIPEEEQMGLLRVISGVLQLGNIVFKKERNTDQASMPDNTAAQKVSHLLGINVTDFTRGILTPRIKVGRDYVQKAQTKEQADFAIEALAKATYERMFRWLVLRINKALDKTKRQGASFIGILDIAGFEIFDLNSFEQLCINYTNEKLQQLFNHTMFILEQEEYQREGIEWNFIDFGLDLQPCIDLIEKPAGPPGILALLDEECWFPKATDKSFVEKVMQEQGTHPKFQKPKQLKDKADFCIIHYAGKVDYKADEWLMKNMDPLNDNIATLLHQSSDKFVSELWKDVDRIIGLDQVAGMSETALPGAFKTRKGMFRTVGQLYKEQLAKLMATLRNTNPNFVRCIIPNHEKKAGKLDPHLVLDQLRCNGVLEGIRICRQGFPNRVVFQEFRQRYEILTPNSIPKGFMDGKQACVLMIKALELDSNLYRIGQSKVFFRAGVLAHLEEERDLKITDVIIGFQACCRGYLARKAFAKRQQQLTAMKVLQRNCAAYLKLRNWQWWRLFTKVKPLLQVSRQEEEMMAKEEELVKVREKQLAAENRLTEMETLQSQLMAEKLQLQEQLQAETELCAEAEELRARLTAKKQELEEICHDLEARVEEEEERCQHLQAEKKKMQQNIQELEEQLEEEESARQKLQLEKVTTEAKLKKLEEEQIILEDQNCKLAKEKKLLEDRIAEFTTNLTEEEEKSKSLAKLKNKHEAMITDLEERLRREEKQRQELEKTRRKLEGDSTDLSDQIAELQAQIAELKMQLAKKEEELQAALARVEEEAAQKNMALKKIRELESQISELQEDLESERASRNKAEKQKRDLGEELEALKTELEDTLDSTAAQQELRSKREQEVNILKKTLEEEAKTHEAQIQEMRQKHSQAVEELAEQLEQTKRVKANLEKAKQTLENERGELANEVKVLLQGKGDSEHKRKKVEAQLQELQVKFNEGERVRTELADKVTKLQVELDNVTGLLSQSDSKSSKLTKDFSALESQLQDTQELLQEENRQKLSLSTKLKQVEDEKNSFREQLEEEEEAKHNLEKQIATLHAQVADMKKKMEDSVGCLETAEEVKRKLQKDLEGLSQRHEEKVAAYDKLEKTKTRLQQELDDLLVDLDHQRQSACNLEKKQKKFDQLLAEEKTISAKYAEERDRAEAEAREKETKALSLARALEEAMEQKAELERLNKQFRTEMEDLMSSKDDVGKSVHELEKSKRALEQQVEEMKTQLEELEDELQATEDAKLRLEVNLQAMKAQFERDLQGRDEQSEEKKKQLVRQVREMEAELEDERKQRSMAVAARKKLEMDLKDLEAHIDSANKNRDEAIKQLRKLQEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 141 - protein, 787 aa, synthetic construct
MFRQFYLWTCLASGIILGSLFEICLGQYDDGHRGGHAACLRCRRGTCIRGAGEAVHKHAAPRGHLGPADLPGGTLAQRDLGPGQRQLRAGDRTLAGSQPEPLHLGEPHHAAGGAGQLRLPGPRSGRPLAPLQRVGAAGQPAPAQYLLPLREQEGSPICPDRESLCGKLPGIQWHQRPVQAAFLWCQLQHARGGHLSRGHLGDPVCEHQGPAAAQVCRTSLHGGGHRPADLAGPGPAACNSGGVICGRGGGLPPVLCSQQETAGVGVWRPGLPNRQVVEARRWQRDHQELLHLLSQHQDLPRRPLRCCGDPRHQHLPSGLPPGQHCWGTRAWGAPGDSWLWHLQLLPGGEVLLRARRHPGSTVRRAVPHGDRSRCPLLLHRLGAAVCLLHPLLPQVCPQATHLLSDDLPEARPGLPGQLLLFRCPPALAGLHGEPGLRGCLQDPGGSKVGIPSEELGSWKNSRRRRIWKSGQGNGLPSERQSRVHHGGREDAERERLPEAARPAVRVQRPEAGQPPTCHQIVWGLQPGWPAPPHRGVRQIRLPAGLPPREPQSGAWLPGQWRQPQLQLPGPPGAGPHHGRPHLICLADLTGDAVSGRDEARSSGLGSQKHPGSGAEDEDFGFRLVPRCLRGFLREEEPGSDSSMDGNIPFSYLHHAKCMVFWCPAVGDRDPRGKPLSWDSSAALQPSEDRPPDGEARQLQRGDVPPDAAMLEAGAGQKAGVCGHQQRPGEDDGEERLLGPCGVHSILPDLRRPLRGGDTAGGLCPPPSSPPFHMDKQTLWNFPCIYIL

SEQ ID NO: 142 - protein, 1043 aa, synthetic construct
MSRKISKESKKVNISSSLESEDISLETTVPTDDISSSEEREGKVRITRQLIERKELLHNIQLLKIELSQKTMMIDNLKVDYLTKIEELEEKLNDALHQKQLLTLRLDNQLAFQQKDASKYQELMKQEMETILLRQKQLEETNLQLREKAGDVRRNLRDFELTEEQYIKLKAFPEDQLSIPEYVSVRFYELVNPLRKEICELQVKKNILAEELSTNKNQLKQLTETYEEDRKNYSEVQIRCQRLALELADTKQLIQQGDYRQENYDKVKSERDALEQEVIELRRKHEILEASHMIQTKERSELSKEVVTLEQTVTLLQKDKEYLNRQNMELSVRCAHEEDRLERLQAQLEESKKAREEMYEKYVASRDHYKTEYENKLHDELEQIRLKTNQEIDQLRNASREMYERENRNLREARDNAVAEKERAVMAEKDALEKHDQLLDRYRELQLSTESKVTEFLHQSKLKSFESERVQLLQEETARNLTQCQLECEKYQKKLEVLTKEFYSLQASSEKRITELQAQNSEHQARLDIYEKLEKELDEIIMQTAEIENEDEAERVLFSYGYGANVPTTAKRRLKQSVHLARRVLQLEKQNSLILKDLEHRKDQVTQLSQELDRANSLLNQTQQPYRYLIESVRQRDSKIDSLTESIAQLEKDVSNLNKEKSALLQTKNQMALDLEQLLNHREEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 143 - protein, 1043 aa, synthetic construct; identical to SEQ ID NO: 142

SEQ ID NO: 144 - protein, 1185 aa, synthetic construct
MEPRPTAPSSGAPGLAGVGETPSAAALAAARVELPGTAVPSVPEDAAPASRDGGGVRDEGPAAAGDGLGRPLGPTPSQSRFQVDLVSENAGRAAAAAAAAAAAAAAAGAGAGAKQTPADGEASGESEPAKGSEEAKGRFRVNFVDPAASSSAEDSLSDAAGVGVDGPNVSFQNGGDTVLSEGSSLHSGGGGGSGHHQHYYYDTHTNTYYLRTFGHNTMDAVPRIDHYRHTAAQLGEKLLRPSLAELHDELEKEPFEDGFANGEESTPTRDAVVTYTAESKGVVKFGWIKGVLVRCMLNIWGVMLFIRLSWIVGQAGIGLSVLVIMMATVVTTITGLSTSAIATNGFVRGGGAYYLISRSLGPEFGGAIGLIFAFANAVAVAMYVVGFAETVVELLKEHSILMIDEINDIRIIGAITVVILLGISVAGMEWEAKAQIVLLVILLLAIGDFVIGTFIPLESKKPKGFFGYKSEIFNENFGPDFREEETFFSVFAIFFPAATGILAGANISGDLADPQSAIPKGTLLAILITTLVYVGIAVSVGSCVVRDATGNVNDTIVTELTNCTSAACKLNFDFSSCESSPCSYGLMNNFQVMSMVSGFTPLISAGIFSATLSSALASLVSAPKIFQALCKDNIYPAFQMFAKGYGKNNEPLRGYILTFLIALGFILIAELNVIAPIISNFFLASYALINFSVFHASLAKSPGWRPAFKYYNMWISLLGAILCCIVMFVINWWAALLTYVIVLGLYIYVTYKKPDVNWGSSTQALTYLNALQHSIRLSGVEDHVKNFRPQCLVMTGAPNSRPALLHLVHDFTKNVGLMICGHVHMEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 145 - protein, 426 aa, synthetic construct
MAPRAQIQGPLTFGDVAVAFTRIEWRHLDAAQRALYRDVMLENYGNLVSVGLLSSKPKLITQLEQGAEPWTEVREAPSGTHAGGSKVGIPSEELGSWKNSRRRRIWKSGQGNGLPSERQSRVHHGGREDAERERLPEAARPAVRVQRPEAGQPPTCHQIVWGLQPGWPAPPHRGVRQIRLPAGLPPREPQSGAWLPGQWRQPQLQLPGPPGAGPHHGRPHLICLADLTGDAVSGRDEARSSGLGSQKHPGSGAEDEDFGFRLVPRCLRGFLREEEPGSDSSMDGNIPFSYLHHAKCMVFWCPAVGDRDPRGKPLSWDSSAALQPSEDRPPDGEARQLQRGDVPPDAAMLEAGAGQKAGVCGHQQRPGEDDGEERLLGPCGVHSILPDLRRPLRGGDTAGGLCPPPSSPPFHMDKQTLWNFPCIYIL

SEQ ID NO: 146 - protein, 568 aa, synthetic construct
MAPRAQIQGPLTFGDVAVAFTRIEWRHLDAAQRALYRDVMLENYGNLVSVGLLSSKPKLITQLEQGAEPWTEVREAPSGTHAGGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 147 - DNA, 1596 bp, synthetic construct
  source 1..1596: mol_type = other DNA
atggagaccccgtcccagcggcgcgccacccgcagcggggcgcaggccagctccactccgctgtcgcccacccgcatcacccggctgcaggagaaggaggacctgcaggagctcaatgatcgcttggcggtctacatcgaccgtgtgcgctcgctggaaacggagaacgcagggctgcgccttcgcatcaccgagtctgaagaggtggtcagccgcgaggtgtccggcatcaaggccgcctacgaggccgagctcggggatgcccgcaagacccttgactcagtagccaaggagcgcgcccgcctgcagctggagctgagcaaagtgcgtgaggagtttaaggagctgaaagcgcgcaataccaagaaggagggtgacctgatagctgctcaggctcggctgaaggacctggaggctctgctgaactccaaggaggccgcactgagcactgctctcagtgagaagcgcacgctggagggcgagctgcatgatctgcggggccaggtggccaaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 148 - DNA, 1596 bp, synthetic construct; identical to SEQ ID NO: 147
  source 1..1596: mol_type = other DNA

SEQ ID NO: 149 - DNA, 2187 bp, synthetic construct
  source 1..2187: mol_type = other DNA
atggggaagaaactggatctttccaagctcactgatgaagaggcccagcatgtcttggaagttgttcaacgagattttgacctccgaaggaaagaagaggaacggctagaggcgttgaagggcaagattaagaaggaaagctccaagagggagctgctttccgacactgcccatctgaacgagacccactgcgcccgctgcctgcagccctaccagctgcttgtgaatagcaaaaggcagtgcctggaatgtggcctcttcacctgcaaaagctgtggccgcgtccacccggaggagcagggctggatctgtgacccctgccatctggccagagtcgtgaagatcggctcactggagtggtactatgagcatgtgaaagcccgcttcaagaggttcggaagtgccaaggtcatccggtccctccacgggcggctgcagggtggagctgggcctgaactgatatctgaagagagaagtggagacagcgaccagacagatgaggatggagaacctggctcagaggcccaggcccaggcccagccctttggcagcaaaaaaaagcgcctcctctccgtccacgacttcgacttcgagggagactcagatgactccactcagcctcaaggtcactccctgcacctgtcctcagtccctgaggccagggacagcccacagtccctcacagatgagtcctgctcagagaaggcagcccctcacaaggctgagggcctggaggaggctgatactggggcctctgggtgccactcccatccggaagagcagccgaccagcatctcaccttccagacacggcgccctggctgagctctgcccgcctggaggctcccacaggatggccctggggactgctgctgcactcgggtcgaatgtcatcaggaatgagcagctgcccctgcagtacttggccgatgtggacacctctgatgaggaaagcatccgggctcacgtgatggcctcccaccattccaagcggagaggccgggcgtcttctgagagtcagatctttgagctgaataagcatatttcagctgtggaatgcctgctgacctacctggagaacacagttgtgcctcccttggccaaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 150 - DNA, 6015 bp, synthetic construct
  source 1..6015: mol_type = other DNA
atggcacagcaagctgccgataagtatctctatgtggataaaaacttcatcaacaatccgctggcccaggccgactgggctgccaagaagctggtatgggtgccttccgacaagagtggctttgagccagccagcctcaaggaggaggtgggcgaagaggccatcgtggagctggtggagaatgggaagaaggtgaaggtgaacaaggatgacatccagaagatgaacccgcccaagttctccaaggtggaggacatggcagagctcacgtgcctcaacgaagcctcggtgctgcacaacctcaaggagcgttactactcagggctcatctacacctattcaggcctgttctgtgtggtcatcaatccttacaagaacctgcccatctactctgaagagattgtggaaatgtacaagggcaagaagaggcacgagatgccccctcacatctatgccatcacagacaccgcctacaggagtatgatgcaagaccgagaagatcaatccatcttgtgcactggtgaatctggagctggcaagacggagaacaccaagaaggtcatccagtatctggcgtacgtggcgtcctcgcacaagagcaagaaggaccagggcgagctggagcggcagctgctgcaggccaaccccatcctggaggccttcgggaacgccaagaccgtgaagaatgacaactcctcccgcttcggcaaattcattcgcatcaactttgatgtcaatggctacattgttggagccaacattgagacttatcttttggagaaatctcgtgctatccgccaagccaaggaagaacggaccttccacatcttctattatctcctgtctggggctggagagcacctgaagaccgatctcctgttggagccgtacaacaaataccgcttcctgtccaatggacacgtcaccatccccgggcagcaggacaaggacatgttccaggagaccatggaggccatgaggattatgggcatcccagaagaggagcaaatgggcctgctgcgggtcatctcaggggttcttcagctcggcaacatcgtcttcaagaaggagcggaacactgaccaggcgtccatgcccgacaacacagctgcccaaaaggtgtcccatctcttgggtatcaatgtgaccgatttcaccagaggaatcctcaccccgcgcatcaaggtgggacgggattacgtccagaaggcgcagactaaagagcaggctgactttgccatcgaggccttggccaaggcgacctatgagcggatgttccgctggctggtgctgcgcatcaacaaggctctggacaagaccaagaggcagggcgcctccttcatcgggatcctggacattgccggcttcgagatctttgatctgaactcgtttgagcagctgtgcatcaattacaccaatgagaagctgcagcagctcttcaaccacaccatgttcatcctggagcaggaggagtaccagcgcgagggcatcgagtggaacttcatcgactttggcctcgacctgcagccctgcatcgacctcattgagaagccagcaggccccccgggcattctggccctgctggacgaggagtgctggttccccaaagccaccgacaagagcttcgtggagaaggtgatgcaggagcagggcacccaccccaagttccagaagcccaagcagctgaaggacaaagctgatttctgcattatccactatgccggcaaggtggattacaaagctgacgagtggctgatgaagaacatggatcccctgaatgacaacatcgccacactgctccaccagtcctctgacaagtttgtctcggagctgtggaaggatgtggaccgcatcatcggcctggaccaggtggccggcatgtcggagaccgcactgcccggggccttcaagacgcggaagggcatgttccgcactgtggggcagctttacaaggagcagctggccaagctgatggctacgctgaggaacacgaaccccaactttgtccgctgcatcatccccaaccacgagaagaaggccggcaagctggacccgcatctcgtgctggaccagctgcgctgcaacggtgttctcgagggcatccgtatctgccgccagggcttccccaacagggtggtcttccaggagtttcggcagagatatgagatcctgactccaaactccattcccaagggtttcatggacgggaagcaggcgtgcgtgctcatgataaaagccctggagctcgacagcaatctgtaccgcattggccagagcaaagtcttcttccgtgccggtgtgctggcccacctggaggaggagcgagacctgaagatcaccgacgtcatcatagggttccaggcctgctgcaggggctacctggccaggaaagcatttgccaagcggcagcagcagcttaccgccatgaaggtcctccagcggaactgcgctgcctacctgaagctgcggaactggcagtggtggcggctcttcaccaaggtcaagccgctgctgcaggtgagccggcaggaggaggagatgatggccaaggaggaggagctggtgaaggtcagagagaagcagctggctgcggagaacaggctcacggagatggagacgctgcagtctcagctcatggcagagaaattgcagctgcaggagcagctccaggcagaaaccgagctgtgtgccgaggctgaggagctccgggcccgcctgaccgccaagaagcaggaattagaagagatctgccatgacctagaggccagggtggaggaggaggaggagcgctgccagcacctgcaggcggagaagaagaagatgcagcagaacatccaggagcttgaggagcagctggaggaggaggagagcgcccggcagaagctgcagctggagaaggtgaccaccgaggcgaagctgaaaaagctggaggaggagcagatcatcctggaggaccagaactgcaagctggccaaggaaaagaaactgctggaagacagaatagctgagttcaccaccaacctcacagaagaggaggagaaatctaagagcctcgccaagctcaagaacaagcatgaggcaatgatcactgacttggaagagcgcctccgcagggaggagaagcagcgacaggagctggagaagacccgccggaagctggagggagactccacagacctcagcgaccagatcgccgagctccaggcccagatcgcggagctcaagatgcagctggccaagaaagaggaggagctccaggccgccctggccagagtggaagaggaagctgcccagaagaacatggccctcaagaagatccgggagctggaatctcagatctctgaactccaggaagacctggagtctgagcgtgcttccaggaataaagctgagaagcagaaacgggaccttggggaagagctagaggctctgaaaacagagttggaggacacgctggattccacagctgcccagcaggagctcaggtcaaaacgtgagcaggaggtgaacatcctgaagaagaccctggaggaggaggccaagacccacgaggcccagatccaggagatgaggcagaagcactcacaggccgtggaggagctggcggagcagctggagcagacgaagcgggtgaaagcaaacctcgagaaggcaaagcagactctggagaacgagcggggggagctggccaacgaggtgaaggtgctgctgcagggcaaaggggactcggagcacaagcgcaagaaagtggaggcgcagctgcaggagctgcaggtcaagttcaacgagggagagcgcgtgcgcacagagctggccgacaaggtcaccaagctgcaggtggagctggacaacgtgaccgggcttctcagccagtccgacagcaagtccagcaagctcaccaaggacttctccgcgctggagtcccagctgcaggacactcaggagctgctgcaggaggagaaccggcagaagctgagcctgagcaccaagctcaagcaggtggaggacgagaagaattccttccgggagcagctggaggaggaggaggaggccaagcacaacctggagaagcagatcgccaccctccatgcccaggtggccgacatgaaaaagaagatggaggacagtgtggggtgcctggaaactgctgaggaggtgaagaggaagctccagaaggacctggagggcctgagccagcggcacgaggagaaggtggccgcctacgacaagctggagaagaccaagacgcggctgcagcaggagctggacgacctgctggtggacctggaccaccagcgccagagcgcgtgcaacctggagaagaagcagaagaagtttgaccagctcctggcggaggagaagaccatctctgccaagtatgcagaggagcgcgaccgggctgaggcggaggcccgagagaaggagaccaaggctctgtcgctggcccgggccctggaggaagccatggagcagaaggcggagctggagcggctcaacaagcagttccgcacggagatggaggaccttatgagctccaaggatgatgtgggcaagagtgtccacgagctggagaagtccaagcgggccctagagcagcaggtggaggagatgaagacgcagctggaagagctggaggacgagctgcaggccaccgaagatgccaagctgcggttggaggtcaacctgcaggccatgaaggcccagttcgagcgggacctgcagggccgggacgagcagagcgaggagaagaagaagcagctggtcagacaggtgcgggagatggaggcagagctggaggacgagaggaagcagcgctcgatggcagtggccgcccggaagaagctggagatggacctgaaggacctggaggcgcacatcgactcggccaacaagaaccgggacgaagccatcaaacagctgcggaagctgcaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 151 - DNA, 2443 bp, synthetic construct
  source 1..2443: mol_type = other DNA
atgtttcgacagttttatctctggacatgtttagcttcagggatcatcctgggctctctctttgaaatctgcttgggccagtatgatgatggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 152 - DNA, 3132 bp, synthetic construct
  source 1..3132: mol_type = other DNA
atgtctcgaaaaatttcaaaggagtcaaaaaaagtgaacatctctagttctctggaatctgaagatattagtttagaaacaacagttcctacggatgatatttcctcatcagaagagcgagagggcaaagtcagaatcaccaggcagctaattgaacgaaaagaactacttcataatattcagttactaaaaattgagctatcccagaaaactatgatgatcgacaatttgaaagtggattatcttacaaagattgaagaattggaggagaaacttaatgatgcacttcaccagaagcagctactaacattgagattagacaaccaattggcttttcaacagaaagatgccagcaaatatcaagaattaatgaaacaagaaatggaaaccattttgttgagacagaaacaactagaagagacaaatcttcagctaagagaaaaagctggagatgttcgtcgaaacctgcgtgactttgagttgacagaagagcaatatattaaattaaaagcttttcctgaagatcagctttctattcctgaatatgtatctgttcgcttctatgagctagtgaatccattaagaaaggaaatctgtgaactacaagtgaaaaagaatatcctagcagaagaattaagtacaaacaaaaaccaactgaagcagctgacagagacatatgaggaagatcgaaaaaactactctgaagttcaaattagatgtcaacgtttggccttagaattagcagacacaaaacagttaattcagcaaggtgactaccgtcaagagaactatgataaagtcaagagtgaacgtgatgcacttgaacaggaagtaattgagcttaggagaaaacatgaaatacttgaagcctctcacatgattcaaacaaaagaacgaagtgaattatcaaaagaggtagtcaccttagagcaaactgttactttactgcaaaaggataaagaatatcttaatcgccaaaacatggagcttagtgttcgctgtgctcatgaagaggatcgccttgaaagacttcaagctcaactggaagaaagcaaaaaggctagagaagagatgtatgaaaaatatgtagcatccagagaccattataaaacagaatatgaaaataaactacatgatgaactagaacaaatcagattgaaaaccaaccaagaaattgatcaacttcgaaatgcctctagggaaatgtatgaacgagaaaacagaaatctccgagaagcaagggataatgctgtggctgaaaaggaacgagcagtgatggctgaaaaggatgctttagaaaaacacgatcagctcttagacaggtacagagaactacaacttagtacagaaagcaaagtaacagaatttctccatcaaagtaaattaaaatcttttgaaagtgagcgtgttcaacttctgcaagaggaaacagcaagaaatctcacacagtgtcaattggaatgtgaaaaatatcagaaaaaattggaggttttaaccaaagaattttatagtctccaagcctcttctgaaaaacgcattactgaacttcaagcacagaactcagagcatcaagcaaggctagacatttatgagaaactggaaaaagagcttgatgaaataataatgcaaactgcagaaattgaaaatgaagatgaggctgaaagggttcttttttcctacggctatggtgctaatgttcccacaacagccaaaagacgactaaagcaaagtgttcacttggcaagaagagtgcttcaattagaaaaacaaaactcgctgattttaaaagatctggaacatcgaaaggaccaagtaacacagctttcacaagagcttgacagagccaattcgctattaaaccagactcaacagccttacaggtatctcattgaatcagtgcgtcagagagattctaagattgattcactgacggaatctattgcacaacttgagaaagatgtcagcaacttaaataaagaaaagtcagctttactacagacgaagaatcaaatggcattagatttagaacaacttctaaatcatcgtgaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 153 - DNA, 3132 bp, synthetic construct; identical to SEQ ID NO: 152
  source 1..3132: mol_type = other DNA

SEQ ID NO: 154 - DNA, 3558 bp, synthetic construct
  source 1..3558: mol_type = other DNA
atggagccgcggcccacggcgccctcctccggcgccccgggactggccggggtcggggagacgccgtcagccgctgcgctggccgcagccagggtggaactgcccggcacggctgtgccctcggtgccggaggatgctgcgcccgcgagccgggacggcggcggggtccgcgatgagggccccgcggcggccggggacgggctgggcagacccttggggcccaccccgagccagagccgtttccaggtggacctggtttccgagaacgccgggcgggccgctgctgcggcggcggcggcggcggcggcagcggcggcggctggtgctggggcgggggccaagcagacccccgcggacggggaagccagcggcgagagcgagccggctaaaggcagcgaggaagccaagggccgcttccgcgtgaacttcgtggacccagctgcctcctcgtcggctgaagacagcctgtcagatgctgccggggtcggagtcgacgggcccaacgtgagcttccagaacggcggggacacggtgctgagcgagggcagcagcctgcactccggcggcggcggcggcagtgggcaccaccagcactactattatgatacccacaccaacacctactacctgcgcaccttcggccacaacaccatggacgctgtgcccaggatcgatcactaccggcacacagccgcgcagctgggcgagaagctgctccggcctagcctggcggagctccacgacgagctggaaaaggaaccttttgaggatggctttgcaaatggggaagaaagtactccaaccagagatgctgtggtcacgtatactgcagaaagtaaaggagtcgtgaagtttggctggatcaagggtgtattagtacgttgtatgttaaacatttggggtgtgatgcttttcattagattgtcatggattgtgggtcaagctggaataggtctatcagtccttgtaataatgatggccactgttgtgacaactatcacaggattgtctacttcagcaatagcaactaatggatttgtaagaggaggaggagcatattatttaatatctagaagtctagggccagaatttggtggtgcaattggtctaatcttcgcctttgccaacgctgttgcagttgctatgtatgtggttggatttgcagaaaccgtggtggagttgcttaaggaacattccatacttatgatagatgaaatcaatgatatccgaattattggagccattacagtcgtgattcttttaggtatctcagtagctggaatggagtgggaagcaaaagctcagattgttcttttggtgatcctacttcttgctattggtgatttcgtcataggaacatttatcccactggagagcaagaagccaaaagggttttttggttataaatctgaaatatttaatgagaactttgggcccgattttcgagaggaagagactttcttttctgtatttgccatcttttttcctgctgcaactggtattctggctggagcaaatatctcaggtgatcttgcagatcctcagtcagccatacccaaaggaacactcctagccattttaattactacattggtttacgtaggaattgcagtatctgtaggttcttgtgttgttcgagatgccactggaaacgttaatgacactatcgtaacagagctaacaaactgtacttctgcagcctgcaaattaaactttgatttttcatcttgtgaaagcagtccttgttcctatggcctaatgaacaacttccaggtaatgagtatggtgtcaggatttacaccactaatttctgcaggtatattttcagccactctttcttcagcattagcatccctagtgagtgctcccaaaatatttcaggctctatgtaaggacaacatctacccagctttccagatgtttgctaaaggttatgggaaaaataatgaacctcttcgtggctacatcttaacattcttaattgcacttggattcatcttaattgctgaactgaatgttattgcaccaattatctcaaacttcttccttgcatcatatgcattgatcaatttttcagtattccatgcatcacttgcaaaatctccaggatggcgtcctgcattcaaatactacaacatgtggatatcacttcttggagcaattctttgttgcatagtaatgttcgtcattaactggtgggctgcattgctaacatatgtgatagtccttgggctgtatatttatgttacctacaaaaaaccagatgtgaattggggatcctctacacaagccctgacttacctgaatgcactgcagcattcaattcgtctttctggagtggaagaccacgtgaaaaactttaggccacagtgtcttgttatgacaggtgctccaaactcacgtccagctttacttcatcttgttcatgatttcacaaaaaatgttggtttgatgatctgtggccatgtacatatggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 155 - DNA, 1330 bp, synthetic construct
  source 1..1330: mol_type = other DNA
atggccccaagagcccagatccagggaccgctgacatttggggatgtggctgtggcctttacccggattgagtggagacacctggatgctgctcagcgggccctgtacagggatgtgatgctggagaactatgggaatctggtgtctgtggggcttctctcttccaaaccaaaactaattactcagttggagcaaggggcagagccctggactgaggtgcgagaggctccatcaggcacacatgcaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 156 - DNA, 1707 bp, synthetic construct
  source 1..1707: mol_type = other DNA
atggccccaagagcccagatccagggaccgctgacatttggggatgtggctgtggcctttacccggattgagtggagacacctggatgctgctcagcgggccctgtacagggatgtgatgctggagaactatgggaatctggtgtctgtggggcttctctcttccaaaccaaaactaattactcagttggagcaaggggcagagccctggactgaggtgcgagaggctccatcaggcacacatgcagggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag